(19) United States
(12) Patent Application Publication
(10) Pub. No.: US 2025/0090686 A1
(43) Pub. Date: Mar. 20, 2025

(54) Precise Excisions of Portions of Exon 44, 50, and 53 for Treatment of Duchenne Muscular Dystrophy

(71) Applicant: Vertex Pharmaceuticals Incorporated, Boston, MA (US)

(72) Inventors: Tudor Fulga, Boston, MA (US); Yurong Xin, Boston, MA (US); Yi-Li Min, Boston, MA (US); Foram Ashar, Boston, MA (US); Su Wang, Boston, MA (US); Jianming Liu, Boston, MA (US); Yang Guo, Boston, MA (US); D'anna Nelson, Boston, MA (US)

(73) Assignee: Vertex Pharmaceuticals Incorporated, Boston, MA (US)

(21) Appl. No.: 18/826,808

(22) Filed: Sep. 6, 2024

Related U.S. Application Data
(63) Continuation of application No. PCT/US23/63885, filed on Mar. 7, 2023.
(60) Provisional application No. 63/317,818, filed on Mar. 8, 2022.
(60) Provisional application No. 63/374,887, filed on Sep. 7, 2022.

(51) Int. Cl. A61K 48/00; A61P 21/00; C12N 9/22; C12N 15/11; C12N 15/86

(52) U.S. Cl. CPC A61K 48/005; A61P 21/00; C12N 9/22; C12N 15/111; C12N 15/86; C12N 2310/20; C12N 2320/33; C12N 2750/14143

(57) ABSTRACT

Compositions and methods for treating Duchenne Muscular Dystrophy (DMD) and excising small portions of exons 44, 50, and 53 of the DMD gene are encompassed.

This application is a bypass continuation of PCT/US2023/63885, filed Mar. 7, 2023, which claims the benefit of priority to U.S. Provisional Application No. 63/317,818, filed Mar. 8, 2022, and U.S. Provisional Application No. 63/374,887, filed Sep. 7, 2022, all of which are incorporated herein by reference in their entirety.

The instant application contains a Sequence Listing which has been submitted electronically in XML format and is hereby incorporated by reference in its entirety. Said XML copy, created on Mar. 6, 2023, is named 01245-0034-00PCT_Sequence_Listing and is 2,227,275 bytes in size.

### INTRODUCTION AND SUMMARY

Muscular dystrophies (MD) are a group of more than 30 genetic diseases characterized by progressive weakness and degeneration of the skeletal muscles that control movement. Duchenne muscular dystrophy (DMD) is one of the most severe forms of MD that affects approximately 1 in 5000 boys and is characterized by progressive muscle weakness and premature death. Cardiomyopathy and heart failure are common, incurable and lethal features of DMD. The disease is caused by mutations in the gene encoding dystrophin (DMD), which result in loss of expression of dystrophin, causing muscle membrane fragility and progressive muscle wasting.

CRISPR-based genome editing can provide sequence-specific cleavage of genomic DNA using a Cas9 and a guide RNA. For example, a nucleic acid encoding the Cas9 enzyme and a nucleic acid encoding for the appropriate guide RNA can be provided on separate vectors or together on a single vector and administered in vivo or in vitro to knockout or correct a genetic mutation, for example. The approximately 20 nucleotides at the 5′ end of the guide RNA serves as the guide or spacer sequence that can be any sequence complementary to one strand of a genomic target location that has an adjacent protospacer adjacent motif (PAM). The PAM sequence is a short sequence adjacent to the Cas9 nuclease cut site that the Cas9 molecule requires for appropriate binding. The nucleotides 3′ of the guide or spacer sequence of the guide RNA serve as a scaffold sequence for interacting with Cas9. When a guide RNA and a Cas9 are expressed, the guide RNA will bind to Cas9 and direct it to the sequence complementary to the guide sequence, where it will then initiate a double-stranded break (DSB). To repair these breaks, cells typically use an error prone mechanism of non-homologous end joining (NHEJ) which can lead to disruption of function in the target gene through insertions or deletion of codons, shifts in the reading frame, or result in a premature stop codon triggering nonsense-mediated decay. See, e.g., Kumar et al. (2018) Front. Mol. Neurosci. Vol. 11, Article 413.

While gene editing strategies using systems (e.g., CRISPR) for treating DMD have been previously explored, these strategies have focused primarily on either: a) cutting at multiple different sites to excise large portions (e.g., one or more exons) of the dystrophin gene (see, e.g., Ousterout et al., 2015, Nat Commun. 6:6244); or b) cutting in a single site to introduce indels that either result in a frame-shifting mutation and/or that destroy a splice acceptor/donor site in the dystrophin gene (see, e.g., Amoassi et al., 2018, Science, 362(6410):86-91). However, there remains a need for additional alternative and effective gene editing strategies for treating diseases like DMD.

Provided herein are compositions and methods for treating DMD utilizing Cas proteins such as Staphylococcus aureus (SaCas9) and Staphylococcus lugdunensis (SluCas9). In some embodiments, pairs of guide RNAs that excise small portions of the DMD gene are provided, where the nucleic acid encoding the pairs of guide RNAs may be on a single nucleic acid molecule.

Accordingly, the following non-limiting embodiments are provided.
Embodiment 1 is a composition comprising one or more guide RNAs or a nucleic acid encoding one or more guide RNAs, wherein the one or more guide RNAs comprise i) a guide sequence of Table 6; ii) at least 16, 17, 18, 19, or 20 contiguous nucleotides of a guide sequence of Table 6; iii) a guide sequence that is at least 90% identical to a guide sequence of Table 6; or iv) any one of the guide sequence pairs shown in Tables 1B, 1D, 3B, 3D, 5B, and 5D, optionally further comprising a SaCas9 or a nucleic acid encoding a SaCas9 (for SEQ ID NOs: 1-159) or a SluCas9 or a nucleic acid encoding a SluCas9 (for SEQ ID NOs: 200-292, 924-938, or 950-955).
Embodiment 2 is a composition comprising a pair of guide RNAs or a nucleic acid encoding a pair of guide RNAs, wherein the pair of guide RNAs comprise or consist of a first and a second guide sequence, wherein the first and the second guide sequences are selected from any of the following pairs of guide sequences:
   a. For exon 44 with SaCas9, SEQ ID NOs: 1 and 3; 110 and 120; 110 and 121; 110 and 122; 110 and 123; 110 and 124; 110 and 125; 111 and 112; 111 and 113; 111 and 114; 111 and 115; 111 and 116; 111 and 117; 111 and 118; 111 and 119; 111 and 120; 111 and 121; 111 and 122; 111 and 123; 111 and 124; 111 and 125; 112 and 113; 112 and 114; 112 and 115; 112 and 116; 112 and 117; 112 and 118; 112 and 119; 112 and 120; 112 and 121; 112 and 122; 112 and 123; 112 and 124; 112 and 125; 113 and 114; 113 and 115; 113 and 116; 113 and 117; 113 and 118; 113 and 119; 113 and 120; 113 and 121; 113 and 122; 113 and 123; 113 and 124; 113 and 125; 114 and 115; 114 and 116; 114 and 117; 114 and 118; 114 and 119; 114 and 120; 114 and 121; 114 and 122; 114 and 123; 114 and 124; 114 and 125; 115 and 116; 115 and 117; 115 and 118; 115 and 119; 115 and 120; 115 and 121; 115 and 122; 115 and 123; 115 and 124; 115 and 125; 116 and 117; 116 and 118; 116 and 119; 116 and 120; 116 and 121; 116 and 122; 116 and 123; 116 and 124; 116 and 125; 117 and 118; 117 and 119; 117 and 120; 117 and 121; 117 and 122; 117 and 123; 117 and 124; 117 and 125; 118 and 119; 118 and 120; 118 and 121; 118 and 122; 118 and 123; 118 and 124; 118 and 125; 119 and 120; 119 and 121; 119 and 122; 119 and 123; 119 and 124; 119 and 125; 120 and 121; 120 and 122; 120 and 123; 120 and 124; 120 and 125; 121 and 122; 121 and 123; 121 and 124; 121 and 125; 122 and 123; 122 and 124; 122 and 125; 123 and 124; 123 and 125; or 124 and 125;
   b. For exon 44 with SluCas9, SEQ ID NOs: 200 and 201; 200 and 202; 200 and 203; 200 and 204; 200 and 205; 200 and 206; 200 and 207; 200 and 208; 201 and 202; 201 and 203; 201 and 204; 201 and 205; 201 and 206; 201 and 207; 201 and 208; 202 and 203; 202 and 204; 202 and 205; 202 and 206; 202 and 207; 202 and 208; 203 and 204; 203 and 205; 203 and 206; 203 and 207; 203 and 208; 204 and 205; 204 and 206; 204 and 207; 204 and 208; 205 and 206; 205 and 207; 205 and 208; 206 and 207; 206 and 208; 207 and 208;
   c. For exon 50 with SaCas9, SEQ ID NOs: 148 and 149; 148 and 150; 148 and 151; 148 and 152; 148 and 153; 148 and 154; 148 and 155; 148 and 156; 148 and 157; 148 and 158; 148 and 159; 149 and 150; 149 and 151; 149 and 152; 149 and 153; 149 and 154; 149 and 155; 149 and 156; 149 and 157; 149 and 158; 149 and 159; 150 and 151; 150 and 152; 150 and 153; 150 and 154; 150 and 155; 150 and 156; 150 and 157; 150 and 158; 150 and 159; 151 and 152; 151 and 153; 151 and 154; 151 and 155; 151 and 156; 151 and 157; 151 and 158; 151 and 159; 152 and 153; 152 and 154; 152 and 155; 152 and 156; 152 and 157; 152 and 158; 152 and 159; 153 and 154; 153 and 155; 153 and 156; 153 and 157; 153 and 158; 153 and 159; 154 and 155; 154 and 156; 154 and 157; 154 and 158; 154 and 159; 155 and 156; 155 and 157; 155 and 158; 155 and 159; 156 and 157; 156 and 158; 156 and 159; 157 and 158; 157 and 159; or 158 and 159;
   d. For exon 50 with SluCas9, SEQ ID NOs: 231 and 232; 231 and 233; 231 and 234; 231 and 235; 231 and 236; 231 and 237; 231 and 238; 231 and 239; 231 and 240; 231 and 241; 231 and 242; 232 and 233; 232 and 234; 232 and 235; 232 and 236; 232 and 237; 232 and 238; 232 and 239; 232 and 240; 232 and 241; 232 and 242; 233 and 234; 233 and 235; 233 and 236; 233 and 237; 233 and 238; 233 and 239; 233 and 240; 233 and 241; 233 and 242; 234 and 235; 234 and 236; 234 and 237; 234 and 238; 234 and 239; 234 and 240; 234 and 241; 234 and 242; 235 and 236; 235 and 237; 235 and 238; 235 and 239; 235 and 240; 235 and 241; 235 and 242; 236 and 237; 236 and 238; 236 and 239; 236 and 240; 236 and 241; 236 and 242; 237 and 238; 237 and 239; 237 and 240; 237 and 241; 237 and 242; 238 and 239; 238 and 240; 238 and 241; 238 and 242; 239 and 240; 239 and 241; 239 and 242; 240 and 241; 240 and 242; or 241 and 242;
   e. For exon 53 with SaCas9, SEQ ID NOs: 16 and 17; 16 and 18; 16 and 19; 16 and 20; 16 and 21; 16 and 22; 16 and 23; 16 and 24; 16 and 25; 16 and 26; 17 and 18; 17 and 19; 17 and 20; 17 and 21; 17 and 22; 17 and 23; 17 and 24; 17 and 25; 17 and 26; 18 and 19; 18 and 20; 18 and 21; 18 and 22; 18 and 23; 18 and 24; 18 and 25; 18 and 26; 19 and 20; 19 and 21; 19 and 22; 19 and 23; 19 and 24; 19 and 25; 19 and 26; 20 and 21; 20 and 22; 20 and 23; 20 and 24; 20 and 25; 20 and 26; 21 and 22; 21 and 23; 21 and 24; 21 and 25; 21 and 26; 22 and 23; 22 and 24; 22 and 25; 22 and 26; 23 and 24; 23 and 25; 23 and 26; 24 and 25; 24 and 26; 25 and 26; 71 and 72; 71 and 73; 71 and 74; 71 and 75; 71 and 76; 71 and 77; 71 and 78; 71 and 84; 71 and 85; 71 and 86; 71 and 87; 71 and 88; 71 and 89; 71 and 90; 71 and 91; 71 and 92; 71 and 93; 71 and 94; 71 and 95; 71 and 96; 71 and 97; 71 and 98; 71 and 99; 71 and 100; 71 and 101; 71 and 102; 72 and 73; 72 and 74; 72 and 75; 72 and 76; 72 and 77; 72 and 78; 72 and 84; 72 and 85; 72 and 86; 72 and 87; 72 and 88; 72 and 89; 72 and 90; 72 and 91; 72 and 92; 72 and 93; 72 and 94; 72 and 95; 72 and 96; 72 and 97; 72 and 98; 72 and 99; 72 and 100; 72 and 101; 72 and 102; 73 and 74; 73 and 75; 73 and 76; 73 and 77; 73 and 78; 73 and 84; 73 and 85; 73 and 86; 73 and 87; 73 and 88; 73 and 89; 73 and 90; 73 and 91; 73 and 92; 73 and 93; 73 and 94; 73 and 95; 73 and 96; 73 and 97; 73 and 98; 73 and 99; 73 and 100; 73 and 101; 73 and 102; 74 and 75; 74 and 76; 74 and 77; 74 and 78; 74 and 84; 74 and 85; 74 and 86; 74 and 87; 74 and 88; 74 and 89; 74 and 90; 74 and 91; 74 and 92; 74 and 93; 74 and 94; 74 and 95; 74 and 96; 74 and 97; 74 and 98; 74 and 99; 74 and 100; 74 and 101; 74 and 102; 75 and 76; 75 and 77; 75 and 78; 75 and 84; 75 and 85; 75 and 86; 75 and 87; 75 and 88; 75 and 89; 75 and 90; 75 and 91; 75 and 92; 75 and 93; 75 and 94; 75 and 95; 75 and 96; 75 and 97; 75 and 98; 75 and 99; 75 and 100; 75 and 101; 75 and 102; 76 and 77; 76 and 78; 76 and 84; 76 and 85; 76 and 86; 76 and 87; 76 and 88; 76 and 89; 76 and 90; 76 and 91; 76 and 92; 76 and 93; 76 and 94; 76 and 95; 76 and 96; 76 and 97; 76 and 98; 76 and 99; 76 and 100; 76 and 101; 76 and 102; 77 and 78; 77 and 84; 77 and 85; 77 and 86; 77 and 87; 77 and 88; 77 and 89; 77 and 90; 77 and 91; 77 and 92; 77 and 93; 77 and 94; 77 and 95; 77 and 96; 77 and 97; 77 and 98; 77 and 99; 77 and 100; 77 and 101; 77 and 102; 78 and 84; 78 and 85; 78 and 86; 78 and 87; 78 and 88; 78 and 89; 78 and 90; 78 and 91; 78 and 92; 78 and 93; 78 and 94; 78 and 95; 78 and 96; 78 and 97; 78 and 98; 78 and 99; 78 and 100; 78 and 101; 78 and 102; 84 and 85; 84 and 86; 84 and 87; 84 and 88; 84 and 89; 84 and 90; 84 and 91; 84 and 92; 84 and 93; 84 and 94; 84 and 95; 84 and 96; 84 and 97; 84 and 98; 84 and 99; 84 and 100; 84 and 101; 84 and 102; 85 and 86; 85 and 87; 85 and 88; 85 and 89; 85 and 90; 85 and 91; 85 and 92; 85 and 93; 85 and 94; 85 and 95; 85 and 96; 85 and 97; 85 and 98; 85 and 99; 85 and 100; 85 and 101; 85 and 102; 86 and 87; 86 and 88; 86 and 89; 86 and 90; 86 and 91; 86 and 92; 86 and 93; 86 and 94; 86 and 95; 86 and 96; 86 and 97; 86 and 98; 86 and 99; 86 and 100; 86 and 101; 86 and 102; 87 and 88; 87 and 89; 87 and 90; 87 and 91; 87 and 92; 87 and 93; 87 and 94; 87 and 95; 87 and 96; 87 and 97; 87 and 98; 87 and 99; 87 and 100; 87 and 101; 87 and 102; 88 and 89; 88 and 90; 88 and 91; 88 and 92; 88 and 93; 88 and 94; 88 and 95; 88 and 96; 88 and 97; 88 and 98; 88 and 99; 88 and 100; 88 and 101; 88 and 102; 89 and 90; 89 and 91; 89 and 92; 89 and 93; 89 and 94; 89 and 95; 89 and 96; 89 and 97; 89 and 98; 89 and 99; 89 and 100; 89 and 101; 89 and 102; 90 and 91; 90 and 92; 90 and 93; 90 and 94; 90 and 95; 90 and 96; 90 and 97; 90 and 98; 90 and 99; 90 and 100; 90 and 101; 90 and 102; 91 and 92; 91 and 93; 91 and 94; 91 and 95; 91 and 96; 91 and 97; 91 and 98; 91 and 99; 91 and 100; 91 and 101; 91 and 102; 92 and 93; 92 and 94; 92 and 95; 92 and 96; 92 and 97; 92 and 98; 92 and 99; 92 and 100; 92 and 101; 92 and 102; 93 and 94; 93 and 95; 93 and 96; 93 and 97; 93 and 98; 93 and 99; 93 and 100; 93 and 101; 93 and 102; 94 and 95; 94 and 96; 94 and 97; 94 and 98; 94 and 99; 94 and 100; 94 and 101; 94 and 102; 95 and 96; 95 and 97; 95 and 98; 95 and 99; 95 and 100; 95 and 101; 95 and 102; 96 and 97; 96 and 98; 96 and 99; 96 and 100; 96 and 101; 96 and 102; 97 and 98; 97 and 99; 97 and 100; 97 and 101; 97 and 102; 98 and 99; 98 and 100; 98 and 101; 98 and 102; 99 and 100; 99 and 101; 99 and 102; 100 and 101; 100 and 102; or 101 and 102;
   f. For exon 53 with SluCas9, SEQ ID NOs: 270 and 271; 270 and 272; 270 and 273; 270 and 274; 270 and 275; 270 and 276; 270 and 277; 270 and 278; 270 and 279; 270 and 280; 270 and 281; 270 and 282; 270 and 283; 270 and 284; 270 and 285; 270 and 286; 270 and 287; 270 and 288; 270 and 289; 270 and 290; 270 and 291; 270 and 292; 271 and 272; 271 and 273; 271 and 274; 271 and 275; 271 and 276; 271 and 277; 271 and 278; 271 and 279; 271 and 280; 271 and 281; 271 and 282; 271 and 283; 271 and 284; 271 and 285; 271 and 286; 271 and 287; 271 and 288; 271 and 289; 271 and 290; 271 and 291; 271 and 292; 272 and 273; 272 and 274; 272 and 275; 272 and 276; 272 and 277; 272 and 278; 272 and 279; 272 and 280; 272 and 281; 272 and 282; 272 and 283; 272 and 284; 272 and 285; 272 and 286; 272 and 287; 272 and 288; 272 and 289; 272 and 290; 272 and 291; 272 and 292; 273 and 274; 273 and 275; 273 and 276; 273 and 277; 273 and 278; 273 and 279; 273 and 280; 273 and 281; 273 and 282; 273 and 283; 273 and 284; 273 and 285; 273 and 286; 273 and 287; 273 and 288; 273 and 289; 273 and 290; 273 and 291; 273 and 292; 274 and 275; 274 and 276; 274 and 277; 274 and 278; 274 and 279; 274 and 280; 274 and 281; 274 and 282; 274 and 283; 274 and 284; 274 and 285; 274 and 286; 274 and 287; 274 and 288; 274 and 289; 274 and 290; 274 and 291; 274 and 292; 275 and 276; 275 and 277; 275 and 278; 275 and 279; 275 and 280; 275 and 281; 275 and 282; 275 and 283; 275 and 284; 275 and 285; 275 and 286; 275 and 287; 275 and 288; 275 and 289; 275 and 290; 275 and 291; 275 and 292; 276 and 277; 276 and 278; 276 and 279; 276 and 280; 276 and 281; 276 and 282; 276 and 283; 276 and 284; 276 and 285; 276 and 286; 276 and 287; 276 and 288; 276 and 289; 276 and 290; 276 and 291; 276 and 292; 277 and 278; 277 and 279; 277 and 280; 277 and 281; 277 and 282; 277 and 283; 277 and 284; 277 and 285; 277 and 286; 277 and 287; 277 and 288; 277 and 289; 277 and 290; 277 and 291; 277 and 292; 278 and 279; 278 and 280; 278 and 281; 278 and 282; 278 and 283; 278 and 284; 278 and 285; 278 and 286; 278 and 287; 278 and 288; 278 and 289; 278 and 290; 278 and 291; 278 and 292; 279 and 280; 279 and 281; 279 and 282; 279 and 283; 279 and 284; 279 and 285; 279 and 286; 279 and 287; 279 and 288; 279 and 289; 279 and 290; 279 and 291; 279 and 292; 280 and 281; 280 and 282; 280 and 283; 280 and 284; 280 and 285; 280 and 286; 280 and 287; 280 and 288; 280 and 289; 280 and 290; 280 and 291; 280 and 292; 281 and 282; 281 and 283; 281 and 284; 281 and 285; 281 and 286; 281 and 287; 281 and 288; 281 and 289; 281 and 290; 281 and 291; 281 and 292; 282 and 283; 282 and 284; 282 and 285; 282 and 286; 282 and 287; 282 and 288; 282 and 289; 282 and 290; 282 and 291; 282 and 292; 283 and 284; 283 and 285; 283 and 286; 283 and 287; 283 and 288; 283 and 289; 283 and 290; 283 and 291; 283 and 292; 284 and 285; 284 and 286; 284 and 287; 284 and 288; 284 and 289; 284 and 290; 284 and 291; 284 and 292; 285 and 286; 285 and 287; 285 and 288; 285 and 289; 285 and 290; 285 and 291; 285 and 292; 286 and 287; 286 and 288; 286 and 289; 286 and 290; 286 and 291; 286 and 292; 287 and 288; 287 and 289; 287 and 290; 287 and 291; 287 and 292; 288 and 289; 288 and 290; 288 and 291; 288 and 292; 289 and 290; 289 and 291; 289 and 292; 290 and 291; 290 and 292; 291 and 292; 924 and 270; 924 and 272; 924 and 273; 924 and 274; 924 and 275; 924 and 276; 924 and 277; 924 and 278; 924 and 279; 924 and 280; 924 and 281; 924 and 282; 924 and 283; 924 and 284; 924 and 285; 924 and 286; 924 and 287; 924 and 288; 924 and 289; 924 and 290; 924 and 291; 924 and 292; 925 and 270; 925 and 272; 925 and 273; 925 and 274; 925 and 275; 925 and 276; 925 and 277; 925 and 278; 925 and 279; 925 and 280; 925 and 281; 925 and 282; 925 and 283; 925 and 284; 925 and 285; 925 and 286; 925 and 287; 925 and 288; 925 and 289; 925 and 290; 925 and 291; 925 and 292; 926 and 270; 926 and 272; 926 and 273; 926 and 274; 926 and 275; 926 and 276; 926 and 277; 926 and 278; 926 and 279; 926 and 280; 926 and 281; 926 and 282; 926 and 283; 926 and 284; 926 and 285; 926 and 286; 926 and 287; 926 and 288; 926 and 289; 926 and 290; 926 and 291; 926 and 292; 927 and 270; 927 and 272; 927 and 273; 927 and 274; 927 and 275; 927 and 276; 927 and 277; 927 and 278; 927 and 279; 927 and 280; 927 and 281; 927 and 282; 927 and 283; 927 and 284; 927 and 285; 927 and 286; 927 and 287; 927 and 288; 927 and 289; 927 and 290; 927 and 291; 927 and 292; 928 and 270; 928 and 272; 928 and 273; 928 and 274; 928 and 275; 928 and 276; 928 and 277; 928 and 278; 928 and 279; 928 and 280; 928 and 281; 928 and 282; 928 and 283; 928 and 284; 928 and 285; 928 and 286; 928 and 287; 928 and 288; 928 and 289; 928 and 290; 928 and 291; 928 and 292; 929 and 270; 929 and 272; 929 and 273; 929 and 274; 929 and 275; 929 and 276; 929 and 277; 929 and 278; 929 and 279; 929 and 280; 929 and 281; 929 and 282; 929 and 283; 929 and 284; 929 and 285; 929 and 286; 929 and 287; 929 and 288; 929 and 289; 929 and 290; 929 and 291; 929 and 292; 930 and 270; 930 and 272; 930 and 273; 930 and 274; 930 and 275; 930 and 276; 930 and 277; 930 and 278; 930 and 279; 930 and 280; 930 and 281; 930 and 282; 930 and 283; 930 and 284; 930 and 285; 930 and 286; 930 and 287; 930 and 288; 930 and 289; 930 and 290; 930 and 291; 930 and 292; 931 and 270; 931 and 272; 931 and 273; 931 and 274; 931 and 275; 931 and 276; 931 and 277; 931 and 278; 931 and 279; 931 and 280; 931 and 281; 931 and 282; 931 and 283; 931 and 284; 931 and 285; 931 and 286; 931 and 287; 931 and 288; 931 and 289; 931 and 290; 931 and 291; 931 and 292; 932 and 270; 932 and 272; 932 and 273; 932 and 274; 932 and 275; 932 and 276; 932 and 277; 932 and 278; 932 and 279; 932 and 280; 932 and 281; 932 and 282; 932 and 283; 932 and 284; 932 and 285; 932 and 286; 932 and 287; 932 and 288; 932 and 289; 932 and 290; 932 and 291; 932 and 292; 933 and 270; 933 and 272; 933 and 273; 933 and 274; 933 and 275; 933 and 276; 933 and 277; 933 and 278; 933 and 279; 933 and 280; 933 and 281; 933 and 282; 933 and 283; 933 and 284; 933 and 285; 933 and 286; 933 and 287; 933 and 288; 933 and 289; 933 and 290; 933 and 291; 933 and 292; 934 and 270; 934 and 272; 934 and 273; 934 and 274; 934 and 275; 934 and 276; 934 and 277; 934 and 278; 934 and 279; 934 and 280; 934 and 281; 934 and 282; 934 and 283; 934 and 284; 934 and 285; 934 and 286; 934 and 287; 934 and 288; 934 and 289; 934 and 290; 934 and 291; 934 and 292; 935 and 270; 935 and 272; 935 and 273; 935 and 274; 935 and 275; 935 and 276; 935 and 277; 935 and 278; 935 and 279; 935 and 280; 935 and 281; 935 and 282; 935 and 283; 935 and 284; 935 and 285; 935 and 286; 935 and 287; 935 and 288; 935 and 289; 935 and 290; 935 and 291; 935 and 292; 936 and 270; 936 and 272; 936 and 273; 936 and 274; 936 and 275; 936 and 276; 936 and 277; 936 and 278; 936 and 279; 936 and 280; 936 and 281; 936 and 282; 936 and 283; 936 and 284; 936 and 285; 936 and 286; 936 and 287; 936 and 288; 936 and 289; 936 and 290; 936 and 291; 936 and 292; 937 and 270; 937 and 272; 937 and 273; 937 and 274; 937 and 275; 937 and 276; 937 and 277; 937 and 278; 937 and 279; 937 and 280; 937 and 281; 937 and 282; 937 and 283; 937 and 284; 937 and 285; 937 and 286; 937 and 287; 937 and 288; 937 and 289; 937 and 290; 937 and 291; 937 and 292; 938 and 270; 938 and 272; 938 and 273; 938 and 274; 938 and 275; 938 and 276; 938 and 277; 938 and 278; 938 and 279; 938 and 280; 938 and 281; 938 and 282; 938 and 283; 938 and 284; 938 and 285; 938 and 286; 938 and 287; 938 and 288; 938 and 289; 938 and 290; 938 and 291; 938 and 292; 950 and 275; 951 and 275; 952 and 275; 953 and 275; 954 and 275; or 955 and 275.
   g. For exon 44 with SluCas9, SEQ ID NOs: 200 and 203; 200 and 204; 202 and 203; 202 and 205; 202 and 206; 202 and 207; 204 and 208; 204 and 205; 204 and 206; 204 and 207; or 204 and 208;
   h. For exon 50 with SluCas9, SEQ ID NOs: 231 and 232; 231 and 234; 231 and 236; 231 and 237; 236 and 233; 236 and 235; 236 and 238; 236 and 240; or 236 and 241;
   i. For exon 53 with SaCas9, SEQ ID NOs: 86 and 96; 87 and 96; 88 and 97; 89 and 96; 90 and 97; 92 and 77; 92 and 78; 92 and 96; 92 and 99; 93 and 98; 93 and 102; 94 and 100; 95 and 77; 95 and 78; 95 and 99; 96 and 100; 97 and 98; 97 and 102; 98 and 73; or 102 and 73; and
   j. For exon 53 with SluCas9, SEQ ID NOs: 278 and 290; 278 and 292; 281 and 291; 283 and 290; 283 and 292; 287 and 291; 272 and 290; 290 and 291; or 272 and 292.
Embodiment 3 is a composition comprising: one or more nucleic acid molecules encoding
   a. a SaCas9 or SluCas9; and
   b. a first guide sequence and a second guide sequence, wherein the first and the second guide sequences are selected from any of the following pairs of guide sequences: SEQ ID NO: 1 and SEQ ID NO: 3; SEQ ID NO: 16 and SEQ ID NO: 17; SEQ ID NO: 16 and SEQ ID NO: 18; SEQ ID NO: 16 and SEQ ID NO: 19; SEQ ID NO: 16 and SEQ ID NO: 20; SEQ ID NO: 16 and SEQ ID NO: 21; SEQ ID NO: 16 and SEQ ID NO: 22; SEQ ID NO: 16 and SEQ ID NO: 23; SEQ ID NO: 16 and SEQ ID NO: 24; SEQ ID NO: 16 and SEQ ID NO: 25; SEQ ID NO: 16 and SEQ ID NO: 26; SEQ ID NO: 17 and SEQ ID NO: 18; SEQ ID NO: 17 and SEQ ID NO: 19; SEQ ID NO: 17 and SEQ ID NO: 20; SEQ ID NO: 17 and SEQ ID NO: 21; SEQ ID NO: 17 and SEQ ID NO: 22; SEQ ID NO: 17 and SEQ ID NO: 23; SEQ ID NO: 17 and SEQ ID NO: 24; SEQ ID NO: 17 and SEQ ID NO: 25; SEQ ID NO: 17 and SEQ ID NO: 26; SEQ ID NO: 18 and SEQ ID NO: 19; SEQ ID NO: 18 and SEQ ID NO: 20; SEQ ID NO: 18 and SEQ ID NO: 21; SEQ ID NO: 18 and SEQ ID NO: 22; SEQ ID NO: 18 and SEQ ID NO: 23; SEQ ID NO: 18 and SEQ ID NO: 24; SEQ ID NO: 18 and SEQ ID NO: 25; SEQ ID NO: 18 and SEQ ID NO: 26; SEQ ID NO: 19 and SEQ ID NO: 20; SEQ ID NO: 19 and SEQ ID NO: 21; SEQ ID NO: 19 and SEQ ID NO: 22; SEQ ID NO: 19 and SEQ ID NO: 23; SEQ ID NO: 19 and SEQ ID NO: 24; SEQ ID NO: 19 and SEQ ID NO: 25; SEQ ID NO: 19 and SEQ ID NO: 26; SEQ ID NO: 20 and SEQ ID NO: 21; SEQ ID NO: 20 and SEQ ID NO: 22; SEQ ID NO: 20 and SEQ ID NO: 23; SEQ ID NO: 20 and SEQ ID NO: 24; SEQ ID NO: 20 and SEQ ID NO: 25; SEQ ID NO: 20 and SEQ ID NO: 26; SEQ ID NO: 21 and SEQ ID NO: 22; SEQ ID NO: 21 and SEQ ID NO: 23; SEQ ID NO: 21 and SEQ ID NO: 24; SEQ ID NO: 21 and SEQ ID NO: 25; SEQ ID NO: 21 and SEQ ID NO: 26; SEQ ID NO: 22 and SEQ ID NO: 23; SEQ ID NO: 22 and SEQ ID NO: 24; SEQ ID NO: 22 and SEQ ID NO: 25; SEQ ID NO: 22 and SEQ ID NO: 26; SEQ ID NO: 23 and SEQ ID NO: 24; SEQ ID NO: 23 and SEQ ID NO: 25; SEQ ID NO: 23 and SEQ ID NO: 26; SEQ ID NO: 24 and SEQ ID NO: 25; SEQ ID NO: 24 and SEQ ID NO: 26; SEQ ID NO: 25 and SEQ ID NO: 26; SEQ ID NO: 110 and SEQ ID NO: 120; SEQ ID NO: 110 and SEQ ID NO: 121; SEQ ID NO: 110 and SEQ ID NO: 122; SEQ ID NO: 110 and SEQ ID NO: 123; SEQ ID NO: 110 and SEQ ID NO: 124; SEQ ID NO: 110 and SEQ ID NO: 125; SEQ ID NO: 111 and SEQ ID NO: 112; SEQ ID NO: 111 and SEQ ID NO: 113; SEQ ID NO: 111 and SEQ ID NO: 114; SEQ ID NO: 111 and SEQ ID NO: 115; SEQ ID NO: 111 and SEQ ID NO: 116; SEQ ID NO: 111 and SEQ ID NO: 117; SEQ ID NO: 111 and SEQ ID NO: 118; SEQ ID NO: 111 and SEQ ID NO: 119; SEQ ID NO: 111 and SEQ ID NO: 120; SEQ ID NO: 111 and SEQ ID NO: 121; SEQ ID NO: 111 and SEQ ID NO: 122; SEQ ID NO: 111 and SEQ ID NO: 123; SEQ ID NO: 111 and SEQ ID NO: 124; SEQ ID NO: 111 and SEQ ID NO: 125; SEQ ID NO: 112 and SEQ ID NO: 113; SEQ ID NO: 112 and SEQ ID NO: 114; SEQ ID NO: 112 and SEQ ID NO: 115; SEQ ID NO: 112 and SEQ ID NO: 116; SEQ ID NO: 112 and SEQ ID NO: 117; SEQ ID NO: 112 and SEQ ID NO: 118; SEQ ID NO: 112 and SEQ ID NO: 119; SEQ ID NO: 112 and SEQ ID NO: 120; SEQ ID NO: 112 and SEQ ID NO: 121; SEQ ID NO: 112 and SEQ ID NO: 122; SEQ ID NO: 112 and SEQ ID NO: 123; SEQ ID NO: 112 and SEQ ID NO: 124; SEQ ID NO: 112 and SEQ ID NO: 125; SEQ ID NO: 113 and SEQ ID NO: 114; SEQ ID NO: 113 and SEQ ID NO: 115; SEQ ID NO: 113 and SEQ ID NO: 116; SEQ ID NO: 113 and SEQ ID NO: 117; SEQ ID NO: 113 and SEQ ID NO: 118; SEQ ID NO: 113 and SEQ ID NO: 119; SEQ ID NO: 113 and SEQ ID NO: 120; SEQ ID NO: 113 and SEQ ID NO: 121; SEQ ID NO: 113 and SEQ ID NO: 122; SEQ ID NO: 113 and SEQ ID NO: 123; SEQ ID NO: 113 and SEQ ID NO: 124; SEQ ID NO: 113 and SEQ ID NO: 125; SEQ ID NO: 114 and SEQ ID NO: 115; SEQ ID NO: 114 and SEQ ID NO: 116; SEQ ID NO: 114 and SEQ ID NO: 117; SEQ ID NO: 114 and SEQ ID NO: 118; SEQ ID NO: 114 and SEQ ID NO: 119; SEQ ID NO: 114 and SEQ ID NO: 120; SEQ ID NO: 114 and SEQ ID NO: 121; SEQ ID NO: 114 and SEQ ID NO: 122; SEQ ID NO: 114 and SEQ ID NO: 123; SEQ ID NO: 114 and SEQ ID NO: 124; SEQ ID NO: 114 and SEQ ID NO: 125; SEQ ID NO: 115 and SEQ ID NO: 116; SEQ ID NO: 115 and SEQ ID NO: 117; SEQ ID NO: 115 and SEQ ID NO: 118; SEQ ID NO: 115 and SEQ ID NO: 119; SEQ ID NO: 115 and SEQ ID NO: 120; SEQ ID NO: 115 and SEQ ID NO: 121; SEQ ID NO: 115 and SEQ ID NO: 122; SEQ ID NO: 115 and SEQ ID NO: 123; SEQ ID NO: 115 and SEQ ID NO: 124; SEQ ID NO: 115 and SEQ ID NO: 125; SEQ ID NO: 116 and SEQ ID NO: 117; SEQ ID NO: 116 and SEQ ID NO: 118; SEQ ID NO: 116 and SEQ ID NO: 119; SEQ ID NO: 116 and SEQ ID NO: 120; SEQ ID NO: 116 and SEQ ID NO: 121; SEQ ID NO: 116 and SEQ ID NO: 122; SEQ ID NO: 116 and SEQ ID NO: 123; SEQ ID NO: 116 and SEQ ID NO: 124; SEQ ID NO: 116 and SEQ ID NO: 125; SEQ ID NO: 117 and SEQ ID NO: 118; SEQ ID NO: 117 and SEQ ID NO: 119; SEQ ID NO: 117 and SEQ ID NO: 120; SEQ ID NO: 117 and SEQ ID NO: 121; SEQ ID NO: 117 and SEQ ID NO: 122; SEQ ID NO: 117 and SEQ ID NO: 123; SEQ ID NO: 117 and SEQ ID NO: 124; SEQ ID NO: 117 and SEQ ID NO: 125; SEQ ID NO: 118 and SEQ ID NO: 119; SEQ ID NO: 118 and SEQ ID NO: 120; SEQ ID NO: 118 and SEQ ID NO: 121; SEQ ID NO: 118 and SEQ ID NO: 122; SEQ ID NO: 118 and SEQ ID NO: 123; SEQ ID NO: 118 and SEQ ID NO: 124; SEQ ID NO: 118 and SEQ ID NO: 125; SEQ ID NO: 119 and SEQ ID NO: 120; SEQ ID NO: 119 and SEQ ID NO: 121; SEQ ID NO: 119 and SEQ ID NO: 122; SEQ ID NO: 119 and SEQ ID NO: 123; SEQ ID NO: 119 and SEQ ID NO: 124; SEQ ID NO: 119 and SEQ ID NO: 125; SEQ ID NO: 120 and SEQ ID NO: 121; SEQ ID NO: 120 and SEQ ID NO: 122; SEQ ID NO: 120 and SEQ ID NO: 123; SEQ ID NO: 120 and SEQ ID NO: 124; SEQ ID NO: 120 and SEQ ID NO: 125; SEQ ID NO: 121 and SEQ ID NO: 122; SEQ ID NO: 121 and SEQ ID NO: 123; SEQ ID NO: 121 and SEQ ID NO: 124; SEQ ID NO: 121 and SEQ ID NO: 125; SEQ ID NO: 122 and SEQ ID NO: 123; SEQ ID NO: 122 and SEQ ID NO: 124; SEQ ID NO: 122 and SEQ ID NO: 125; SEQ ID NO: 123 and SEQ ID NO: 124; SEQ ID NO: 123 and SEQ ID NO: 125; SEQ ID NO: 124 and SEQ ID NO: 125; SEQ ID NO: 148 and SEQ ID NO: 149; SEQ ID NO: 148 and SEQ ID NO: 150; SEQ ID NO: 148 and SEQ ID NO: 151; SEQ ID NO: 148 and SEQ ID NO: 152; SEQ ID NO: 148 and SEQ ID NO: 153; SEQ ID NO: 148 and SEQ ID NO: 154; SEQ ID NO: 148 and SEQ ID NO: 155; SEQ ID NO: 148 and SEQ ID NO: 156; SEQ ID NO: 148 and SEQ ID NO: 157; SEQ ID NO: 148 and SEQ ID NO: 158; SEQ ID NO: 148 and SEQ ID NO: 159; SEQ ID NO: 149 and SEQ ID NO: 150; SEQ ID NO: 149 and SEQ ID NO: 151; SEQ ID NO: 149 and SEQ ID NO: 152; SEQ ID NO: 149 and SEQ ID NO: 153; SEQ ID NO: 149 and SEQ ID NO: 154; SEQ ID NO: 149 and SEQ ID NO: 155; SEQ ID NO: 149 and SEQ ID NO: 156; SEQ ID NO: 149 and SEQ ID NO: 157; SEQ ID NO: 149 and SEQ ID NO: 158; SEQ ID NO: 149 and SEQ ID NO: 159; SEQ ID NO: 150 and SEQ ID NO: 151; SEQ ID NO: 150 and SEQ ID NO: 152; SEQ ID NO: 150 and SEQ ID NO: 153; SEQ ID NO: 150 and SEQ ID NO: 154; SEQ ID NO: 150 and SEQ ID NO: 155; SEQ ID NO: 150 and SEQ ID NO: 156; SEQ ID NO: 150 and SEQ ID NO: 157; SEQ ID NO: 150 and SEQ ID NO: 158; SEQ ID NO: 150 and SEQ ID NO: 159; SEQ ID NO: 151 and SEQ ID NO: 152; SEQ ID NO: 151 and SEQ ID NO: 153; SEQ ID NO: 151 and SEQ ID NO: 154; SEQ ID NO: 151 and SEQ ID NO: 155; SEQ ID NO: 151 and SEQ ID NO: 156; SEQ ID NO: 151 and SEQ ID NO: 157; SEQ ID NO: 151 and SEQ ID NO: 158; SEQ ID NO: 151 and SEQ ID NO: 159; SEQ ID NO: 152 and SEQ ID NO: 153; SEQ ID NO: 152 and SEQ ID NO: 154; SEQ ID NO: 152 and SEQ ID NO: 155; SEQ ID NO: 152 and SEQ ID NO: 156; SEQ ID NO: 152 and SEQ ID NO: 157; SEQ ID NO: 152 and SEQ ID NO: 158; SEQ ID NO: 152 and SEQ ID NO: 159; SEQ ID NO: 153 and SEQ ID NO: 154; SEQ ID NO: 153 and SEQ ID NO: 155; SEQ ID NO: 153 and SEQ ID NO: 156; SEQ ID NO: 153 and SEQ ID NO: 157; SEQ ID NO: 153 and SEQ ID NO: 158; SEQ ID NO: 153 and SEQ ID NO: 159; SEQ ID NO: 154 and SEQ ID NO: 155; SEQ ID NO: 154 and SEQ ID NO: 156; SEQ ID NO: 154 and SEQ ID NO: 157; SEQ ID NO: 154 and SEQ ID NO: 158; SEQ ID NO: 154 and SEQ ID NO: 159; SEQ ID NO: 155 and SEQ ID NO: 156; SEQ ID NO: 155 and SEQ ID NO: 157; SEQ ID NO: 155 and SEQ ID NO: 158; SEQ ID NO: 155 and SEQ ID NO: 159; SEQ ID NO: 156 and SEQ ID NO: 157; SEQ ID NO: 156 and SEQ ID NO: 158; SEQ ID NO: 156 and SEQ ID NO: 159; SEQ ID NO: 157 and SEQ ID NO: 158; SEQ ID NO: 157 and SEQ ID NO: 159; SEQ ID NO: 158 and SEQ ID NO: 159; SEQ ID NO: 71 and SEQ ID NO: 72; SEQ ID NO: 71 and SEQ ID NO: 73; SEQ ID NO: 71 and SEQ ID NO: 74; SEQ ID NO: 71 and SEQ ID NO: 75; SEQ ID NO: 71 and SEQ ID NO: 76; SEQ ID NO: 71 and SEQ ID NO: 77; SEQ ID NO: 71 and SEQ ID NO: 78; SEQ ID NO: 71 and SEQ ID NO: 84; SEQ ID NO: 71 and SEQ ID NO: 85; SEQ ID NO: 71 and SEQ ID NO: 86; SEQ ID NO: 71 and SEQ ID NO: 87; SEQ ID NO: 71 and SEQ ID NO: 88; SEQ ID NO: 71 and SEQ ID NO: 89; SEQ ID NO: 71 and SEQ ID NO: 90; SEQ ID NO: 71 and SEQ ID NO: 91; SEQ ID NO: 71 and SEQ ID NO: 92; SEQ ID NO: 71 and SEQ ID NO: 93; SEQ ID NO: 71 and SEQ ID NO: 94; SEQ ID NO: 71 and SEQ ID NO: 95; SEQ ID NO: 71 and SEQ ID NO: 96; SEQ ID NO: 71 and SEQ ID NO: 97; SEQ ID NO: 71 and SEQ ID NO: 98; SEQ ID NO: 71 and SEQ ID NO: 99; SEQ ID NO: 71 and SEQ ID NO: 100; SEQ ID NO: 71 and SEQ ID NO: 101; SEQ ID NO: 71 and SEQ ID NO: 102; SEQ ID NO: 72 and SEQ ID NO: 73; SEQ ID NO: 72 and SEQ ID NO: 74; SEQ ID NO: 72 and SEQ ID NO: 75; SEQ ID NO: 72 and SEQ ID NO: 76; SEQ ID NO: 72 and SEQ ID NO: 77; SEQ ID NO: 72 and SEQ ID NO: 78; SEQ ID NO: 72 and SEQ ID NO: 84; SEQ ID NO: 72 and SEQ ID NO: 85; SEQ ID NO: 72 and SEQ ID NO: 86; SEQ ID NO: 72 and SEQ ID NO: 87; SEQ ID NO: 72 and SEQ ID NO: 88; SEQ ID NO: 72 and SEQ ID NO: 89; SEQ ID NO: 72 and SEQ ID NO: 90; SEQ ID NO: 72 and SEQ ID NO: 91; SEQ ID NO: 72 and SEQ ID NO: 92; SEQ ID NO: 72 and SEQ ID NO: 93; SEQ ID NO: 72 and SEQ ID NO: 94; SEQ ID NO: 72 and SEQ ID NO: 95; SEQ ID NO: 72 and SEQ ID NO: 96; SEQ ID NO: 72 and SEQ ID NO: 97; SEQ ID NO: 72 and SEQ ID NO: 98; SEQ ID NO: 72 and SEQ ID NO: 99; SEQ ID NO: 72 and SEQ ID NO: 100; SEQ ID NO: 72 and SEQ ID NO: 101; SEQ ID NO: 72 and SEQ ID NO: 102; SEQ ID NO: 73 and SEQ ID NO: 74; SEQ ID NO: 73 and SEQ ID NO: 75; SEQ ID NO: 73 and SEQ ID NO: 76; SEQ ID NO: 73 and SEQ ID NO: 77; SEQ ID NO: 73 and SEQ ID NO: 78; SEQ ID NO: 73 and SEQ ID NO: 84; SEQ ID NO: 73 and SEQ ID NO: 85; SEQ ID NO: 73 and SEQ ID NO: 86; SEQ ID NO: 73 and SEQ ID NO: 87; SEQ ID NO: 73 and SEQ ID NO: 88; SEQ ID NO: 73 and SEQ ID NO: 89; SEQ ID NO: 73 and SEQ ID NO: 90; SEQ ID NO: 73 and SEQ ID NO: 91; SEQ ID NO: 73 and SEQ ID NO: 92; SEQ ID NO: 73 and SEQ ID NO: 93; SEQ ID NO: 73 and SEQ ID NO: 94; SEQ ID NO: 73 and SEQ ID NO: 95; SEQ ID NO: 73 and SEQ ID NO: 96; SEQ ID NO: 73 and SEQ ID NO: 97; SEQ ID NO: 73 and SEQ ID NO: 98; SEQ ID NO: 73 and SEQ ID NO: 99; SEQ ID NO: 73 and SEQ ID NO: 100; SEQ ID NO: 73 and SEQ ID NO: 101; SEQ ID NO: 73 and SEQ ID NO: 102; SEQ ID NO: 74 and SEQ ID NO: 75; SEQ ID NO: 74 and SEQ ID NO: 76; SEQ ID NO: 74 and SEQ ID NO: 77; SEQ ID NO: 74 and SEQ ID NO: 78; SEQ ID NO: 74 and SEQ ID NO: 84; SEQ ID NO: 74 and SEQ ID NO: 85; SEQ ID NO: 74 and SEQ ID NO: 86; SEQ ID NO: 74 and SEQ ID NO: 87; SEQ ID NO: 74 and SEQ ID NO: 88; SEQ ID NO: 74 and SEQ ID NO: 89; SEQ ID NO: 74 and SEQ ID NO: 90; SEQ ID NO: 74 and SEQ ID NO: 91; SEQ ID NO: 74 and SEQ ID NO: 92; SEQ ID NO: 74 and SEQ ID NO: 93; SEQ ID NO: 74 and SEQ ID NO: 94; SEQ ID NO: 74 and SEQ ID NO: 95; SEQ ID NO: 74 and SEQ ID NO: 96; SEQ ID NO: 74 and SEQ ID NO: 97; SEQ ID NO: 74 and SEQ ID NO: 98; SEQ ID NO: 74 and SEQ ID NO: 99; SEQ ID NO: 74 and SEQ ID NO: 100; SEQ ID NO: 74 and SEQ ID NO: 101; SEQ ID NO: 74 and SEQ ID NO: 102; SEQ ID NO: 75 and SEQ ID NO: 76; SEQ ID NO: 75 and SEQ ID NO: 77; SEQ ID NO: 75 and SEQ ID NO: 78; SEQ ID NO: 75 and SEQ ID NO: 84; SEQ ID NO: 75 and SEQ ID NO: 85; SEQ ID NO: 75 and SEQ ID NO: 86; SEQ ID NO: 75 and SEQ ID NO: 87; SEQ ID NO: 75 and SEQ ID NO: 88; SEQ ID NO: 75 and SEQ ID NO: 89; SEQ ID NO: 75 and SEQ ID NO: 90; SEQ ID NO: 75 and SEQ ID NO: 91; SEQ ID NO: 75 and SEQ ID NO: 92; SEQ ID NO: 75 and SEQ ID NO: 93; SEQ ID NO: 75 and SEQ ID NO: 94; SEQ ID NO: 75 and SEQ ID NO: 95; SEQ ID NO: 75 and SEQ ID NO: 96; SEQ ID NO: 75 and SEQ ID NO: 97; SEQ ID NO: 75 and SEQ ID NO: 98; SEQ ID NO: 75 and SEQ ID NO: 99; SEQ ID NO: 75 and SEQ ID NO: 100; SEQ ID NO: 75 and SEQ ID NO: 101; SEQ ID NO: 75 and SEQ ID NO: 102; SEQ ID NO: 76 and SEQ ID NO: 77; SEQ ID NO: 76 and SEQ ID NO: 78; SEQ ID NO: 76 and SEQ ID NO: 84; SEQ ID NO: 76 and SEQ ID NO: 85; SEQ ID NO: 76 and SEQ ID NO: 86; SEQ ID NO: 76 and SEQ ID NO: 87; SEQ ID NO: 76 and SEQ ID NO: 88; SEQ ID NO: 76 and SEQ ID NO: 89; SEQ ID NO: 76 and SEQ ID NO: 90; SEQ ID NO: 76 and SEQ ID NO: 91; SEQ ID NO: 76 and SEQ ID NO: 92; SEQ ID NO: 76 and SEQ ID NO: 93; SEQ ID NO: 76 and SEQ ID NO: 94; SEQ ID NO: 76 and SEQ ID NO: 95; SEQ ID NO: 76 and SEQ ID NO: 96; SEQ ID NO: 76 and SEQ ID NO: 97; SEQ ID NO: 76 and SEQ ID NO: 98; SEQ ID NO: 76 and SEQ ID NO: 99; SEQ ID NO: 76 and SEQ ID NO: 100; SEQ ID NO: 76 and SEQ ID NO: 101; SEQ ID NO: 76 and SEQ ID NO: 102; SEQ ID NO: 77 and SEQ ID NO: 78; SEQ ID NO: 77 and SEQ ID NO: 84; SEQ ID NO: 77 and SEQ ID NO: 85; SEQ ID NO: 77 and SEQ ID NO: 86; SEQ ID NO: 77 and SEQ ID NO: 87; SEQ ID NO: 77 and SEQ ID NO: 88; SEQ ID NO: 77 and SEQ ID NO: 89; SEQ ID NO: 77 and SEQ ID NO: 90; SEQ ID NO: 77 and SEQ ID NO: 91; SEQ ID NO: 77 and SEQ ID NO: 92; SEQ ID NO: 77 and SEQ ID NO: 93; SEQ ID NO: 77 and SEQ ID NO: 94; SEQ ID NO: 77 and SEQ ID NO: 95; SEQ ID NO: 77 and SEQ ID NO: 96; SEQ ID NO: 77 and SEQ ID NO: 97; SEQ ID NO: 77 and SEQ ID NO: 98; SEQ ID NO: 77 and SEQ ID NO: 99; SEQ ID NO: 77 and SEQ ID NO: 100; SEQ ID NO: 77 and SEQ ID NO: 101; SEQ ID NO: 77 and SEQ ID NO: 102; SEQ ID NO: 78 and SEQ ID NO: 84; SEQ ID NO: 78 and SEQ ID NO: 85; SEQ ID NO: 78 and SEQ ID NO: 86; SEQ ID NO: 78 and SEQ ID NO: 87; SEQ ID NO: 78 and SEQ ID NO: 88; SEQ ID NO: 78 and SEQ ID NO: 89; SEQ ID NO: 78 and SEQ ID NO: 90; SEQ ID NO: 78 and SEQ ID NO: 91; SEQ ID NO: 78 and SEQ ID NO: 92; SEQ ID NO: 78 and SEQ ID NO: 93; SEQ ID NO: 78 and SEQ ID NO: 94; SEQ ID NO: 78 and SEQ ID NO: 95; SEQ ID NO: 78 and SEQ ID NO: 96; SEQ ID NO: 78 and SEQ ID NO: 97; SEQ ID NO: 78 and SEQ ID NO: 98; SEQ ID NO: 78 and SEQ ID NO: 99; SEQ ID NO: 78 and SEQ ID NO: 100; SEQ ID NO: 78 and SEQ ID NO: 101; SEQ ID NO: 78 and SEQ ID NO: 102; SEQ ID NO: 84 and SEQ ID NO: 85; SEQ ID NO: 84 and SEQ ID NO: 86; SEQ ID NO: 84 and SEQ ID NO: 87; SEQ ID NO: 84 and SEQ ID NO: 88; SEQ ID NO: 84 and SEQ ID NO: 89; SEQ ID NO: 84 and SEQ ID NO: 90; SEQ ID NO: 84 and SEQ ID NO: 91; SEQ ID NO: 84 and SEQ ID NO: 92; SEQ ID NO: 84 and SEQ ID NO: 93; SEQ ID NO: 84 and SEQ ID NO: 94; SEQ ID NO: 84 and SEQ ID NO: 95; SEQ ID NO: 84 and SEQ ID NO: 96; SEQ ID NO: 84 and SEQ ID NO: 97; SEQ ID NO: 84 and SEQ ID NO: 98; SEQ ID NO: 84 and SEQ ID NO: 99; SEQ ID NO: 84 and SEQ ID NO: 100; SEQ ID NO: 84 and SEQ ID NO: 101; SEQ ID NO: 84 and SEQ ID NO: 102; SEQ ID NO: 85 and SEQ ID NO: 86; SEQ ID NO: 85 and SEQ ID NO: 87; SEQ ID NO: 85 and SEQ ID NO: 88; SEQ ID NO: 85 and SEQ ID NO: 89; SEQ ID NO: 85 and SEQ ID NO: 90; SEQ ID NO: 85 and SEQ ID NO: 91; SEQ ID NO: 85 and SEQ ID NO: 92; SEQ ID NO: 85 and SEQ ID NO: 93; SEQ ID NO: 85 and SEQ ID NO: 94; SEQ ID NO: 85 and SEQ ID NO: 95; SEQ ID NO: 85 and SEQ ID NO: 96; SEQ ID NO: 85 and SEQ ID NO: 97; SEQ ID NO: 85 and SEQ ID NO: 98; SEQ ID NO: 85 and SEQ ID NO: 99; SEQ ID NO: 85 and SEQ ID NO: 100; SEQ ID NO: 85 and SEQ ID NO: 101; SEQ ID NO: 85 and SEQ ID NO: 102; SEQ ID NO: 86 and SEQ ID NO: 87; SEQ ID NO: 86 and SEQ ID NO: 88; SEQ ID NO: 86 and SEQ ID NO: 89; SEQ ID NO: 86 and SEQ ID NO: 90; SEQ ID NO: 86 and SEQ ID NO: 91; SEQ ID NO: 86 and SEQ ID NO: 92; SEQ ID NO: 86 and SEQ ID NO: 93; SEQ ID NO: 86 and SEQ ID NO: 94; SEQ ID NO: 86 and SEQ ID NO: 95; SEQ ID NO: 86 and SEQ ID NO: 96; SEQ ID NO: 86 and SEQ ID NO: 97; SEQ ID NO: 86 and SEQ ID NO: 98; SEQ ID NO: 86 and SEQ ID NO: 99; SEQ ID NO: 86 and SEQ ID NO: 100; SEQ ID NO: 86 and SEQ ID NO: 101; SEQ ID NO: 86 and SEQ ID NO: 102; SEQ ID NO: 87 and SEQ ID NO: 88; SEQ ID NO: 87 and SEQ ID NO: 89; SEQ ID NO: 87 and SEQ ID NO: 90; SEQ ID NO: 87 and SEQ ID NO: 91; SEQ ID NO: 87 and SEQ ID NO: 92; SEQ ID NO: 87 and SEQ ID NO: 93; SEQ ID NO: 87 and SEQ ID NO: 94; SEQ ID NO: 87 and SEQ ID NO: 95; SEQ ID NO: 87 and SEQ ID NO: 96; SEQ ID NO: 87 and SEQ ID NO: 97; SEQ ID NO: 87 and SEQ ID NO: 98; SEQ ID NO: 87 and SEQ ID NO: 99; SEQ ID NO: 87 and SEQ ID NO: 100; SEQ ID NO: 87 and SEQ ID NO: 101; SEQ ID NO: 87 and SEQ ID NO: 102; SEQ ID NO: 88 and SEQ ID NO: 89; SEQ ID NO: 88 and SEQ ID NO: 90; SEQ ID NO: 88 and SEQ ID NO: 91; SEQ ID NO: 88 and SEQ ID NO: 92; SEQ ID NO: 88 and SEQ ID NO: 93; SEQ ID NO: 88 and SEQ ID NO: 94; SEQ ID NO: 88 and SEQ ID NO: 95; SEQ ID NO: 88 and SEQ ID NO: 96; SEQ ID NO: 88 and SEQ ID NO: 97; SEQ ID NO: 88 and SEQ ID NO: 98; SEQ ID NO: 88 and SEQ ID NO: 99; SEQ ID NO: 88 and SEQ ID NO: 100; SEQ ID NO: 88 and SEQ ID NO: 101; SEQ ID NO: 88 and SEQ ID NO: 102; SEQ ID NO: 89 and SEQ ID NO: 90; SEQ ID NO: 89 and SEQ ID NO: 91; SEQ ID NO: 89 and SEQ ID NO: 92; SEQ ID NO: 89 and SEQ ID NO: 93; SEQ ID NO: 89 and SEQ ID NO: 94; SEQ ID NO: 89 and SEQ ID NO: 95; SEQ ID NO: 89 and SEQ ID NO: 96; SEQ ID NO: 89 and SEQ ID NO: 97; SEQ ID NO: 89 and SEQ ID NO: 98; SEQ ID NO: 89 and SEQ ID NO: 99; SEQ ID NO: 89 and SEQ ID NO: 100; SEQ ID NO: 89 and SEQ ID NO: 101; SEQ ID NO: 89 and SEQ ID NO: 102; SEQ ID NO: 90 and SEQ ID NO: 91; SEQ ID NO: 90 and SEQ ID NO: 92; SEQ ID NO: 90 and SEQ ID NO: 93; SEQ ID NO: 90 and SEQ ID NO: 94; SEQ ID NO: 90 and SEQ ID NO: 95; SEQ ID NO: 90 and SEQ ID NO: 96; SEQ ID NO: 90 and SEQ ID NO: 97; SEQ ID NO: 90 and SEQ ID NO: 98; SEQ ID NO: 90 and SEQ ID NO: 99; SEQ ID NO: 90 and SEQ ID NO: 100; SEQ ID NO: 90 and SEQ ID NO: 101; SEQ ID NO: 90 and SEQ ID NO: 102; SEQ ID NO: 91 and SEQ ID NO: 92; SEQ ID NO: 91 and SEQ ID NO: 93; SEQ ID NO: 91 and SEQ ID NO: 94; SEQ ID NO: 91 and SEQ ID NO: 95; SEQ ID NO: 91 and SEQ ID NO: 96; SEQ ID NO: 91 and SEQ ID NO: 97; SEQ ID NO: 91 and SEQ ID NO: 98; SEQ ID NO: 91 and SEQ ID NO: 99; SEQ ID NO: 91 and SEQ ID NO: 100; SEQ ID NO: 91 and SEQ ID NO: 101; SEQ ID NO: 91 and SEQ ID NO: 102; SEQ ID NO: 92 and SEQ ID NO: 93; SEQ ID NO: 92 and SEQ ID NO: 94; SEQ ID NO: 92 and SEQ ID NO: 95; SEQ ID NO: 92 and SEQ ID NO: 96; SEQ ID NO: 92 and SEQ ID NO: 97; SEQ ID NO: 92 and SEQ ID NO: 98; SEQ ID NO: 92 and SEQ ID NO: 99; SEQ ID NO: 92 and SEQ ID NO: 100; SEQ ID NO: 92 and SEQ ID NO: 101; SEQ ID NO: 92 and SEQ ID NO: 102; SEQ ID NO: 93 and SEQ ID NO: 94; SEQ ID NO: 93 and SEQ ID NO: 95; SEQ ID NO: 93 and SEQ ID NO: 96; SEQ ID NO: 93 and SEQ ID NO: 97; SEQ ID NO: 93 and SEQ ID NO: 98; SEQ ID NO: 93 and SEQ ID NO: 99; SEQ ID NO: 93 and SEQ ID NO: 100; SEQ ID NO: 93 and SEQ ID NO: 101; SEQ ID NO: 93 and SEQ ID NO: 102; SEQ ID NO: 94 and SEQ ID NO: 95; SEQ ID NO: 94 and SEQ ID NO: 96; SEQ ID NO: 94 and SEQ ID NO: 97; SEQ ID NO: 94 and SEQ ID NO: 98; SEQ ID NO: 94 and SEQ ID NO: 99; SEQ ID NO: 94 and SEQ ID NO: 100; SEQ ID NO: 94 and SEQ ID NO: 101; SEQ ID NO: 94 and SEQ ID NO: 102; SEQ ID NO: 95 and SEQ ID NO: 96; SEQ ID NO: 95 and SEQ ID NO: 97; SEQ ID NO: 95 and SEQ ID NO: 98; SEQ ID NO: 95 and SEQ ID NO: 99; SEQ ID NO: 95 and SEQ ID NO: 100; SEQ ID NO: 95 and SEQ ID NO: 101; SEQ ID NO: 95 and SEQ ID NO: 102; SEQ ID NO: 96 and SEQ ID NO: 97; SEQ ID NO: 96 and SEQ ID NO: 98; SEQ ID NO: 96 and SEQ ID NO: 99; SEQ ID NO: 96 and SEQ ID NO: 100; SEQ ID NO: 96 and SEQ ID NO: 101; SEQ ID NO: 96 and SEQ ID NO: 102; SEQ ID NO: 97 and SEQ ID NO: 98; SEQ ID NO: 97 and SEQ ID NO: 99; SEQ ID NO: 97 and SEQ ID NO: 100; SEQ ID NO: 97 and SEQ ID NO: 101; SEQ ID NO: 97 and SEQ ID NO: 102; SEQ ID NO: 98 and SEQ ID NO: 99; SEQ ID NO: 98 and SEQ ID NO: 100; SEQ ID NO: 98 and SEQ ID NO: 101; SEQ ID NO: 98 and SEQ ID NO: 102; SEQ ID NO: 99 and SEQ ID NO: 100; SEQ ID NO: 99 and SEQ ID NO: 101; SEQ ID NO: 99 and SEQ ID NO: 102; SEQ ID NO: 100 and SEQ ID NO: 101; SEQ ID NO: 100 and SEQ ID NO: 102; SEQ ID NO: 101 and SEQ ID NO: 102; SEQ ID NO: 200 and SEQ ID NO: 201; SEQ ID NO: 200 and SEQ ID NO: 202; SEQ ID NO: 200 and SEQ ID NO: 203; SEQ ID NO: 200 and SEQ ID NO: 204; SEQ ID NO: 200 and SEQ ID NO: 205; SEQ ID NO: 200 and SEQ ID NO: 206; SEQ ID NO: 200 and SEQ ID NO: 207; SEQ ID NO: 200 and SEQ ID NO: 208; SEQ ID NO: 201 and SEQ ID NO: 202; SEQ ID NO: 201 and SEQ ID NO: 203; SEQ ID NO: 201 and SEQ ID NO: 204; SEQ ID NO: 201 and SEQ ID NO: 205; SEQ ID NO: 201 and SEQ ID NO: 206; SEQ ID NO: 201 and SEQ ID NO: 207; SEQ ID NO: 201 and SEQ ID NO: 208; SEQ ID NO: 202 and SEQ ID NO: 203; SEQ ID NO: 202 and SEQ ID NO: 204; SEQ ID NO: 202 and SEQ ID NO: 205; SEQ ID NO: 202 and SEQ ID NO: 206; SEQ ID NO: 202 and SEQ ID NO: 207; SEQ ID NO: 202 and SEQ ID NO: 208; SEQ ID NO: 203 and SEQ ID NO: 204; SEQ ID NO: 203 and SEQ ID NO: 205; SEQ ID NO: 203 and SEQ ID NO: 206; SEQ ID NO: 203 and SEQ ID NO: 207; SEQ ID NO: 203 and SEQ ID NO: 208; SEQ ID NO: 204 and SEQ ID NO: 205; SEQ ID NO: 204 and SEQ ID NO: 206; SEQ ID NO: 204 and SEQ ID NO: 207; SEQ ID NO: 204 and SEQ ID NO: 208; SEQ ID NO: 205 and SEQ ID NO: 206; SEQ ID NO: 205 and SEQ ID NO: 207; SEQ ID NO: 205 and SEQ ID NO: 208; SEQ ID NO: 206 and SEQ ID NO: 207; SEQ ID NO: 206 and SEQ ID NO: 208; SEQ ID NO: 207 and SEQ ID NO: 208; SEQ ID NO: 231 and SEQ ID NO: 232; SEQ ID NO: 231 and SEQ ID NO: 233; SEQ ID NO: 231 and SEQ ID NO: 234; SEQ ID NO: 231 and SEQ ID NO: 235; SEQ ID NO: 231 and SEQ ID NO: 236; SEQ ID NO: 231 and SEQ ID NO: 237; SEQ ID NO: 231 and SEQ ID NO: 238; SEQ ID NO: 231 and SEQ ID NO: 239; SEQ ID NO: 231 and SEQ ID NO: 240; SEQ ID NO: 231 and SEQ ID NO: 241; SEQ ID NO: 231 and SEQ ID NO: 242; SEQ ID NO: 232 and SEQ ID NO: 233; SEQ ID NO: 232 and SEQ ID NO: 234; SEQ ID NO: 232 and SEQ ID NO: 235; SEQ ID NO: 232 and SEQ ID NO: 236; SEQ ID NO: 232 and SEQ ID NO: 237; SEQ ID NO: 232 and SEQ ID NO: 238; SEQ ID NO: 232 and SEQ ID NO: 239; SEQ ID NO: 232 and SEQ ID NO: 240; SEQ ID NO: 232 and SEQ ID NO: 241; SEQ ID NO: 232 and SEQ ID NO: 242; SEQ ID NO: 233 and SEQ ID NO: 234; SEQ ID NO: 233 and SEQ ID NO: 235; SEQ ID NO: 233 and SEQ ID NO: 236; SEQ ID NO: 233 and SEQ ID NO: 237; SEQ ID NO: 233 and SEQ ID NO: 238; SEQ ID NO: 233 and SEQ ID NO: 239; SEQ ID NO: 233 and SEQ ID NO: 240; SEQ ID NO: 233 and SEQ ID NO: 241; SEQ ID NO: 233 and SEQ ID NO: 242; SEQ ID NO: 234 and SEQ ID NO: 235; SEQ ID NO: 234 and SEQ ID NO: 236; SEQ ID NO: 234 and SEQ ID NO: 237; SEQ ID NO: 234 and SEQ ID NO: 238; SEQ ID NO: 234 and SEQ ID NO: 239; SEQ ID NO: 234 and SEQ ID NO: 240; SEQ ID NO: 234 and SEQ ID NO: 241; SEQ ID NO: 234 and SEQ ID NO: 242; SEQ ID NO: 235 and SEQ ID NO: 236; SEQ ID NO: 235 and SEQ ID NO: 237; SEQ ID NO: 235 and SEQ ID NO: 238; SEQ ID NO: 235 and SEQ ID NO: 239; SEQ ID NO: 235 and SEQ ID NO: 240; SEQ ID NO: 235 and SEQ ID NO: 241; SEQ ID NO: 235 and SEQ ID NO: 242; SEQ ID NO: 236 and SEQ ID NO: 237; SEQ ID NO: 236 and SEQ ID NO: 238; SEQ ID NO: 236 and SEQ ID NO: 239; SEQ ID NO: 236 and SEQ ID NO: 240; SEQ ID NO: 236 and SEQ ID NO: 241; SEQ ID NO: 236 and SEQ ID NO: 242; SEQ ID NO: 237 and SEQ ID NO: 238; SEQ ID NO: 237 and SEQ ID NO: 239; SEQ ID NO: 237 and SEQ ID NO: 240; SEQ ID NO: 237 and SEQ ID NO: 241; SEQ ID NO: 237 and SEQ ID NO: 242; SEQ ID NO: 238 and SEQ ID NO: 239; SEQ ID NO: 238 and SEQ ID NO: 240; SEQ ID NO: 238 and SEQ ID NO: 241; SEQ ID NO: 238 and SEQ ID NO: 242; SEQ ID NO: 239 and SEQ ID NO: 240; SEQ ID NO: 239 and SEQ ID NO: 241; SEQ ID NO: 239 and SEQ ID NO: 242; SEQ ID NO: 240 and SEQ ID NO: 241; SEQ ID NO: 240 and SEQ ID NO: 242; SEQ ID NO: 241 and SEQ ID NO: 242; SEQ ID NO: 270 and SEQ ID NO: 271; SEQ ID NO: 270 and SEQ ID NO: 272; SEQ ID NO: 270 and SEQ ID NO: 273; SEQ ID NO: 270 and SEQ ID NO: 274; SEQ ID NO: 270 and SEQ ID NO: 275; SEQ ID NO: 270 and SEQ ID NO: 276; SEQ ID NO: 270 and SEQ ID NO: 277; SEQ ID NO: 270 and SEQ ID NO: 278; SEQ ID NO: 270 and SEQ ID NO: 279; SEQ ID NO: 270 and SEQ ID NO: 280; SEQ ID NO: 270 and SEQ ID NO: 281; SEQ ID NO: 270 and SEQ ID NO: 282; SEQ ID NO: 270 and SEQ ID NO: 283; SEQ ID NO: 270 and SEQ ID NO: 284; SEQ ID NO: 270 and SEQ ID NO: 285; SEQ ID NO: 270 and SEQ ID NO: 286; SEQ ID NO: 270 and SEQ ID NO: 287; SEQ ID NO: 270 and SEQ ID NO: 288; SEQ ID NO: 270 and SEQ ID NO: 289; SEQ ID NO: 270 and SEQ ID NO: 290; SEQ ID NO: 270 and SEQ ID NO: 291; SEQ ID NO: 270 and SEQ ID NO: 292; SEQ ID NO: 271 and SEQ ID NO: 272; SEQ ID NO: 271 and SEQ ID NO: 273; SEQ ID NO: 271 and SEQ ID NO: 274; SEQ ID NO: 271 and SEQ ID NO: 275; SEQ ID NO: 271 and SEQ ID NO: 276; SEQ ID NO: 271 and SEQ ID NO: 277; SEQ ID NO: 271 and SEQ ID NO: 278; SEQ ID NO: 271 and SEQ ID NO: 279; SEQ ID NO: 271 and SEQ ID NO: 280; SEQ ID NO: 271 and SEQ ID NO: 281; SEQ ID NO: 271 and SEQ ID NO: 282; SEQ ID NO: 271 and SEQ ID NO: 283; SEQ ID NO: 271 and SEQ ID NO: 284; SEQ ID NO: 271 and SEQ ID NO: 285; SEQ ID NO: 271 and SEQ ID NO: 286; SEQ ID NO: 271 and SEQ ID NO: 287; SEQ ID NO: 271 and SEQ ID NO: 288; SEQ ID NO: 271 and SEQ ID NO: 289; SEQ ID NO: 271 and SEQ ID NO: 290; SEQ ID NO: 271 and SEQ ID NO: 291; SEQ ID NO: 271 and SEQ ID NO: 292; SEQ ID NO: 272 and SEQ ID NO: 273; SEQ ID NO: 272 and SEQ ID NO: 274; SEQ ID NO: 272 and SEQ ID NO: 275; SEQ ID NO: 272 and SEQ ID NO: 276; SEQ ID NO: 272 and SEQ ID NO: 277; SEQ ID NO: 272 and SEQ ID NO: 278; SEQ ID NO: 272 and SEQ ID NO: 279; SEQ ID NO: 272 and SEQ ID NO: 280; SEQ ID NO: 272 and SEQ ID NO: 281; SEQ ID NO: 272 and SEQ ID NO: 282; SEQ ID NO: 272 and SEQ ID NO: 283; SEQ ID NO: 272 and SEQ ID NO: 284; SEQ ID NO: 272 and SEQ ID NO: 285; SEQ ID NO: 272 and SEQ ID NO: 286; SEQ ID NO: 272 and SEQ ID NO: 287; SEQ ID NO: 272 and SEQ ID NO: 288; SEQ ID NO: 272 and SEQ ID NO: 289; SEQ ID NO: 272 and SEQ ID NO: 290; SEQ ID NO: 272 and SEQ ID NO: 291; SEQ ID NO: 272 and SEQ ID NO: 292; SEQ ID NO: 273 and SEQ ID NO: 274; SEQ ID NO: 273 and SEQ ID NO: 275; SEQ ID NO: 273 and SEQ ID NO: 276; SEQ ID NO: 273 and SEQ ID NO: 277; SEQ ID NO: 273 and SEQ ID NO: 278; SEQ ID NO: 273 and SEQ ID NO: 279; SEQ ID NO: 273 and SEQ ID NO: 280; SEQ ID NO: 273 and SEQ ID NO: 281; SEQ ID NO: 273 and SEQ ID NO: 282; SEQ ID NO: 273 and SEQ ID NO: 283; SEQ ID NO: 273 and SEQ ID NO: 284; SEQ ID NO: 273 and SEQ ID NO: 285; SEQ ID NO: 273 and SEQ ID NO: 286; SEQ ID NO: 273 and SEQ ID NO: 287; SEQ ID NO: 273 and SEQ ID NO: 288; SEQ ID NO: 273 and SEQ ID NO: 289; SEQ ID NO: 273 and SEQ ID NO: 290; SEQ ID NO: 273 and SEQ ID NO: 291; SEQ ID NO: 273 and SEQ ID NO: 292; SEQ ID NO: 274 and SEQ ID NO: 275; SEQ ID NO: 274 and SEQ ID NO: 276; SEQ ID NO: 274 and SEQ ID NO: 277; SEQ ID NO: 274 and SEQ ID NO: 278; SEQ ID NO: 274 and SEQ ID NO: 279; SEQ ID NO: 274 and SEQ ID NO: 280; SEQ ID NO: 274 and SEQ ID NO: 281; SEQ ID NO: 274 and SEQ ID NO: 282; SEQ ID NO: 274 and SEQ ID NO: 283; SEQ ID NO: 274 and SEQ ID NO: 284; SEQ ID NO: 274 and SEQ ID NO: 285; SEQ ID NO: 274 and SEQ ID NO: 286; SEQ ID NO: 274 and SEQ ID NO: 287; SEQ ID NO: 274 and SEQ ID NO: 288; SEQ ID NO: 274 and SEQ ID NO: 289; SEQ ID NO: 274 and SEQ ID NO: 290; SEQ ID NO: 274 and SEQ ID NO: 291; SEQ ID NO: 274 and SEQ ID NO: 292; SEQ ID NO: 275 and SEQ ID NO: 276; SEQ ID NO: 275 and SEQ ID NO: 277; SEQ ID NO: 275 and SEQ ID NO: 278; SEQ ID NO: 275 and SEQ ID NO: 279; SEQ ID NO: 275 and SEQ ID NO: 280; SEQ ID NO: 275 and SEQ ID NO: 281; SEQ ID NO: 275 and SEQ ID NO: 282; SEQ ID NO: 275 and SEQ ID NO: 283; SEQ ID NO: 275 and SEQ ID NO: 284; SEQ ID NO: 275 and SEQ ID NO: 285; SEQ ID NO: 275 and SEQ ID NO: 286; SEQ ID NO: 275 and SEQ ID NO: 287; SEQ ID NO: 275 and SEQ ID NO: 288; SEQ ID NO: 275 and SEQ ID NO: 289; SEQ ID NO: 275 and SEQ ID NO: 290; SEQ ID NO: 275 and SEQ ID NO: 291; SEQ ID NO: 275 and SEQ ID NO: 292; SEQ ID NO: 276 and SEQ ID NO: 277; SEQ ID NO: 276 and SEQ ID NO: 278; SEQ ID NO: 276 and SEQ ID NO: 279; SEQ ID NO: 276 and SEQ ID NO: 280; SEQ ID NO: 276 and SEQ ID NO: 281; SEQ ID NO: 276 and SEQ ID NO: 282; SEQ ID NO: 276 and SEQ ID NO: 283; SEQ ID NO: 276 and SEQ ID NO: 284; SEQ ID NO: 276 and SEQ ID NO: 285; SEQ ID NO: 276 and SEQ ID NO: 286; SEQ ID NO: 276 and SEQ ID NO: 287; SEQ ID NO: 276 and SEQ ID NO: 288; SEQ ID NO: 276 and SEQ ID NO: 289; SEQ ID NO: 276 and SEQ ID NO: 290; SEQ ID NO: 276 and SEQ ID NO: 291; SEQ ID NO: 276 and SEQ ID NO: 292; SEQ ID NO: 277 and SEQ ID NO: 278; SEQ ID NO: 277 and SEQ ID NO: 279; SEQ ID NO: 277 and SEQ ID NO: 280; SEQ ID NO: 277 and SEQ ID NO: 281; SEQ ID NO: 277 and SEQ ID NO: 282; SEQ ID NO: 277 and SEQ ID NO: 283; SEQ ID NO: 277 and SEQ ID NO: 284; SEQ ID NO: 277 and SEQ ID NO: 285; SEQ ID NO: 277 and SEQ ID NO: 286; SEQ ID NO: 277 and SEQ ID NO: 287; SEQ ID NO: 277 and SEQ ID NO: 288; SEQ ID NO: 277 and SEQ ID NO: 289; SEQ ID NO: 277 and SEQ ID NO: 290; SEQ ID NO: 277 and SEQ ID NO: 291; SEQ ID NO: 277 and SEQ ID NO: 292; SEQ ID NO: 278 and SEQ ID NO: 279; SEQ ID NO: 278 and SEQ ID NO: 280; SEQ ID NO: 278 and SEQ ID NO: 281; SEQ ID NO: 278 and SEQ ID NO: 282; SEQ ID NO: 278 and SEQ ID NO: 283; SEQ ID NO: 278 and SEQ ID NO: 284; SEQ ID NO: 278 and SEQ ID NO: 285; SEQ ID NO: 278 and SEQ ID NO: 286; SEQ ID NO: 278 and SEQ ID NO: 287; SEQ ID NO: 278 and SEQ ID NO: 288; SEQ ID NO: 278 and SEQ ID NO: 289; SEQ ID NO: 278 and SEQ ID NO: 290; SEQ ID NO: 278 and SEQ ID NO: 291; SEQ ID NO: 278 and SEQ ID NO: 292; SEQ ID NO: 279 and SEQ ID NO: 280; SEQ ID NO: 279 and SEQ ID NO: 281; SEQ ID NO: 279 and SEQ ID NO: 282; SEQ ID NO: 279 and SEQ ID NO: 283; SEQ ID NO: 279 and SEQ ID NO: 284; SEQ ID NO: 279 and SEQ ID NO: 285; SEQ ID NO: 279 and SEQ ID NO: 286; SEQ ID NO: 279 and SEQ ID NO: 287; SEQ ID NO: 279 and SEQ ID NO: 288; SEQ ID NO: 279 and SEQ ID NO: 289; SEQ ID NO: 279 and SEQ ID NO: 290; SEQ ID NO: 279 and SEQ ID NO: 291; SEQ ID NO: 279 and SEQ ID NO: 292; SEQ ID NO: 280 and SEQ ID NO: 281; SEQ ID NO: 280 and SEQ ID NO: 282; SEQ ID NO: 280 and SEQ ID NO: 283; SEQ ID NO: 280 and SEQ ID NO: 284; SEQ ID NO: 280 and SEQ ID NO: 285; SEQ ID NO: 280 and SEQ ID NO: 286; SEQ ID NO: 280 and SEQ ID NO: 287; SEQ ID NO: 280 and SEQ ID NO: 288; SEQ ID NO: 280 and SEQ ID NO: 289; SEQ ID NO: 280 and SEQ ID NO: 290; SEQ ID NO: 280 and SEQ ID NO: 291; SEQ ID NO: 280 and SEQ ID NO: 292; SEQ ID NO: 281 and SEQ ID NO: 282; SEQ ID NO: 281 and SEQ ID NO: 283; SEQ ID NO: 281 and SEQ ID NO: 284; SEQ ID NO: 281 and SEQ ID NO: 285; SEQ ID NO: 281 and SEQ ID NO: 286; SEQ ID NO: 281 and SEQ ID NO: 287; SEQ ID NO: 281 and SEQ ID NO: 288; SEQ ID NO: 281 and SEQ ID NO: 289; SEQ ID NO: 281 and SEQ ID NO: 290; SEQ ID NO: 281 and SEQ ID NO: 291; SEQ ID NO: 281 and SEQ ID NO: 292; SEQ ID NO: 282 and SEQ ID NO: 283; SEQ ID NO: 282 and SEQ ID NO: 284; SEQ ID NO: 282 and SEQ ID NO: 285; SEQ ID NO: 282 and SEQ ID NO: 286; SEQ ID NO: 282 and SEQ ID NO: 287; SEQ ID NO: 282 and SEQ ID NO: 288; SEQ ID NO: 282 and SEQ ID NO: 289; SEQ ID NO: 282 and SEQ ID NO: 290; SEQ ID NO: 282 and SEQ ID NO: 291; SEQ ID NO: 282 and SEQ ID NO: 292; SEQ ID NO: 283 and SEQ ID NO: 284; SEQ ID NO: 283 and SEQ ID NO: 285; SEQ ID NO: 283 and SEQ ID NO: 286; SEQ ID NO: 283 and SEQ ID NO: 287; SEQ ID NO: 283 and SEQ ID NO: 288; SEQ ID NO: 283 and SEQ ID NO: 289; SEQ ID NO: 283 and SEQ ID NO: 290; SEQ ID NO: 283 and SEQ ID NO: 291; SEQ ID NO: 283 and SEQ ID NO: 292; SEQ ID NO: 284 and SEQ ID NO: 285; SEQ ID NO: 284 and SEQ ID NO: 286; SEQ ID NO: 284 and SEQ ID NO: 287; SEQ ID NO: 284 and SEQ ID NO: 288; SEQ ID NO: 284 and SEQ ID NO: 289; SEQ ID NO: 284 and SEQ ID NO: 290; SEQ ID NO: 284 and SEQ ID NO: 291; SEQ ID NO: 284 and SEQ ID NO: 292; SEQ ID NO: 285 and SEQ ID NO: 286; SEQ ID NO: 285 and SEQ ID NO: 287; SEQ ID NO: 285 and SEQ ID NO: 288; SEQ ID NO: 285 and SEQ ID NO: 289; SEQ ID NO: 285 and SEQ ID NO: 290; SEQ ID NO: 285 and SEQ ID NO: 291; SEQ ID NO: 285 and SEQ ID NO: 292; SEQ ID NO: 286 and SEQ ID NO: 287; SEQ ID NO: 286 and SEQ ID NO: 288; SEQ ID NO: 286 and SEQ ID NO: 289; SEQ ID NO: 286 and SEQ ID NO: 290; SEQ ID NO: 286 and SEQ ID NO: 291; SEQ ID NO: 286 and SEQ ID NO: 292; SEQ ID NO: 287 and SEQ ID NO: 288; SEQ ID NO: 287 and SEQ ID NO: 289; SEQ ID NO: 287 and SEQ ID NO: 290; SEQ ID NO: 287 and SEQ ID NO: 291; SEQ ID NO: 287 and SEQ ID NO: 292; SEQ ID NO: 288 and SEQ ID NO: 289; SEQ ID NO: 288 and SEQ ID NO: 290; SEQ ID NO: 288 and SEQ ID NO: 291; SEQ ID NO: 288 and SEQ ID NO: 292; SEQ ID NO: 289 and SEQ ID NO: 290; SEQ ID NO: 289 and SEQ ID NO: 291; SEQ ID NO: 289 and SEQ ID NO: 292; SEQ ID NO: 290 and SEQ ID NO: 291; SEQ ID NO: 290 and SEQ ID NO: 292; SEQ ID NO: 291 and SEQ ID NO: 292; SEQ ID NO: 270 and SEQ ID NO: 924; SEQ ID NO: 270 and SEQ ID NO: 925; SEQ ID NO: 270 and SEQ ID NO: 926; SEQ ID NO: 270 and SEQ ID NO: 927; SEQ ID NO: 270 and SEQ ID NO: 928; SEQ ID NO: 270 and SEQ ID NO: 929; SEQ ID NO: 270 and SEQ ID NO: 930; SEQ ID NO: 270 and SEQ ID NO: 931; SEQ ID NO: 270 and SEQ ID NO: 932; SEQ ID NO: 270 and SEQ ID NO: 933; SEQ ID NO: 270 and SEQ ID NO: 934; SEQ ID NO: 270 and SEQ ID NO: 935; SEQ ID NO: 270 and SEQ ID NO: 936; SEQ ID NO: 270 and SEQ ID NO: 937; SEQ ID NO: 270 and SEQ ID NO: 938; SEQ ID NO: 272 and SEQ ID NO: 924; SEQ ID NO: 272 and SEQ ID NO: 925; SEQ ID NO: 272 and SEQ ID NO: 926; SEQ ID NO: 272 and SEQ ID NO: 927; SEQ ID NO: 272 and SEQ ID NO: 928; SEQ ID NO: 272 and SEQ ID NO: 929; SEQ ID NO: 272 and SEQ ID NO: 930; SEQ ID NO: 272 and SEQ ID NO: 931; SEQ ID NO: 272 and SEQ ID NO: 932; SEQ ID NO: 272 and SEQ ID NO: 933; SEQ ID NO: 272 and SEQ ID NO: 934; SEQ ID NO: 272 and SEQ ID NO: 935; SEQ ID NO: 272 and SEQ ID NO: 936; SEQ ID NO: 272 and SEQ ID NO: 937; SEQ ID NO: 272 and SEQ ID NO: 938; SEQ ID NO: 273 and SEQ ID NO: 924; SEQ ID NO: 273 and SEQ ID NO: 925; SEQ ID NO: 273 and SEQ ID NO: 926; SEQ ID NO: 273 and SEQ ID NO: 927; SEQ ID NO: 273 and SEQ ID NO: 928; SEQ ID NO: 273 and SEQ ID NO: 929; SEQ ID NO: 273 and SEQ ID NO: 930; SEQ ID NO: 273 and SEQ ID NO: 931; SEQ ID NO: 273 and SEQ ID NO: 932; SEQ ID NO: 273 and SEQ ID NO: 933; SEQ ID NO: 273 and SEQ ID NO: 934; SEQ ID NO: 273 and SEQ ID NO: 935; SEQ ID NO: 273 and SEQ ID NO: 936; SEQ ID NO: 273 and SEQ ID NO: 937; SEQ ID NO: 273 and SEQ ID NO: 938; SEQ ID NO: 274 and SEQ ID NO: 924; SEQ ID NO: 274 and SEQ ID NO: 925; SEQ ID NO: 274 and SEQ ID NO: 926; SEQ ID NO: 274 and SEQ ID NO: 927; SEQ ID NO: 274 and SEQ ID NO: 928; SEQ ID NO: 274 and SEQ ID NO: 929; SEQ ID NO: 274 and SEQ ID NO: 930; SEQ ID NO: 274 and SEQ ID NO: 931; SEQ ID NO: 274 and SEQ ID NO: 932; SEQ ID NO: 274 and SEQ ID NO: 933; SEQ ID NO: 274 and SEQ ID NO: 934; SEQ ID NO: 274 and SEQ ID NO: 935; SEQ ID NO: 274 and SEQ ID NO: 936; SEQ ID NO: 274 and SEQ ID NO: 937; SEQ ID NO: 274 and SEQ ID NO: 938; SEQ ID NO: 275 and SEQ ID NO: 924; SEQ ID NO: 275 and SEQ ID NO: 925; SEQ ID NO: 275 and SEQ ID NO: 926; SEQ ID NO: 275 and SEQ ID NO: 927; SEQ ID NO: 275 and SEQ ID NO: 928; SEQ ID NO: 275 and SEQ ID NO: 929; SEQ ID NO: 275 and SEQ ID NO: 930; SEQ ID NO: 275 and SEQ ID NO: 931; SEQ ID NO: 275 and SEQ ID NO: 932; SEQ ID NO: 275 and SEQ ID NO: 933; SEQ ID NO: 275 and SEQ ID NO: 934; SEQ ID NO: 275 and SEQ ID NO: 935; SEQ ID NO: 275 and SEQ ID NO: 936; SEQ ID NO: 275 and SEQ ID NO: 937; SEQ ID NO: 275 and SEQ ID NO: 938; SEQ ID NO: 276 and SEQ ID NO: 924; SEQ ID NO: 276 and SEQ ID NO: 925; SEQ ID NO: 276 and SEQ ID NO: 926; SEQ ID NO: 276 and SEQ ID NO: 927; SEQ ID NO: 276 and SEQ ID NO: 928; SEQ ID NO: 276 and SEQ ID NO: 929; SEQ ID NO: 276 and SEQ ID NO: 930; SEQ ID NO: 276 and SEQ ID NO: 931; SEQ ID NO: 276 and SEQ ID NO: 932; SEQ ID NO: 276 and SEQ ID NO: 933; SEQ ID NO: 276 and SEQ ID NO: 934; SEQ ID NO: 276 and SEQ ID NO: 935; SEQ ID NO: 276 and SEQ ID NO: 936; SEQ ID NO: 276 and SEQ ID NO: 937; SEQ ID NO: 276 and SEQ ID NO: 938; SEQ ID NO: 277 and SEQ ID NO: 924; SEQ ID NO: 277 and SEQ ID NO: 925; SEQ ID NO: 277 and SEQ ID NO: 926; SEQ ID NO: 277 and SEQ ID NO: 927; SEQ ID NO: 277 and SEQ ID NO: 928; SEQ ID NO: 277 and SEQ ID NO: 929; SEQ ID NO: 277 and SEQ ID NO: 930; SEQ ID NO: 277 and SEQ ID NO: 931; SEQ ID NO: 277 and SEQ ID NO: 932; SEQ ID NO: 277 and SEQ ID NO: 933; SEQ ID NO: 277 and SEQ ID NO: 934; SEQ ID NO: 277 and SEQ ID NO: 935; SEQ ID NO: 277 and SEQ ID NO: 936; SEQ ID NO: 277 and SEQ ID NO: 937; SEQ ID NO: 277 and SEQ ID NO: 938; SEQ ID NO: 278 and SEQ ID NO: 924; SEQ ID NO: 278 and SEQ ID NO: 925; SEQ ID NO: 278 and SEQ ID NO: 926; SEQ ID NO: 278 and SEQ ID NO: 927; SEQ ID NO: 278 and SEQ ID NO: 928; SEQ ID NO: 278 and SEQ ID NO: 929; SEQ ID NO: 278 and SEQ ID NO: 930; SEQ ID NO: 278 and SEQ ID NO: 931; SEQ ID NO: 278 and SEQ ID NO: 932; SEQ ID NO: 278 and SEQ ID NO: 933; SEQ ID NO: 278 and SEQ ID NO: 934; SEQ ID NO: 278 and SEQ ID NO: 935; SEQ ID NO: 278 and SEQ ID NO: 936; SEQ ID NO: 278 and SEQ ID NO: 937; SEQ ID NO: 278 and SEQ ID NO: 938; SEQ ID NO: 279 and SEQ ID NO: 924; SEQ ID NO: 279 and SEQ ID NO: 925; SEQ ID NO: 279 and SEQ ID NO: 926; SEQ ID NO: 279 and SEQ ID NO: 927; SEQ ID NO: 279 and SEQ ID NO: 928; SEQ ID NO: 279 and SEQ ID NO: 929; SEQ ID NO: 279 and SEQ ID NO: 930; SEQ ID NO: 279 and SEQ ID NO: 931; SEQ ID NO: 279 and SEQ ID NO: 932; SEQ ID NO: 279 and SEQ ID NO: 933; SEQ ID NO: 279 and SEQ ID NO: 934; SEQ ID NO: 279 and SEQ ID NO: 935; SEQ ID NO: 279 and SEQ ID NO: 936; SEQ ID NO: 279 and SEQ ID NO: 937; SEQ ID NO: 279 and SEQ ID NO: 938; SEQ ID NO: 280 and SEQ ID NO: 924; SEQ ID NO: 280 and SEQ ID NO: 925; SEQ ID NO: 280 and SEQ ID NO: 926; SEQ ID NO: 280 and SEQ ID NO: 927; SEQ ID NO: 280 and SEQ ID NO: 928; SEQ ID NO: 280 and SEQ ID NO: 929; SEQ ID NO: 280 and SEQ ID NO: 930; SEQ ID NO: 280 and SEQ ID NO: 931; SEQ ID NO: 280 and SEQ ID NO: 932; SEQ ID NO: 280 and SEQ ID NO: 933; SEQ ID NO: 280 and SEQ ID NO: 934; SEQ ID NO: 280 and SEQ ID NO: 935; SEQ ID NO: 280 and SEQ ID NO: 936; SEQ ID NO: 280 and SEQ ID NO: 937; SEQ ID NO: 280 and SEQ ID NO: 938; SEQ ID NO: 281 and SEQ ID NO: 924; SEQ ID NO: 281 and SEQ ID NO: 925; SEQ ID NO: 281 and SEQ ID NO: 926; SEQ ID NO: 281 and SEQ ID NO: 927; SEQ ID NO: 281 and SEQ ID NO: 928; SEQ ID NO: 281 and SEQ ID NO: 929; SEQ ID NO: 281 and SEQ ID NO: 930; SEQ ID NO: 281 and SEQ ID NO: 931; SEQ ID NO: 281 and SEQ ID NO: 932; SEQ ID NO: 281 and SEQ ID NO: 933; SEQ ID NO: 281 and SEQ ID NO: 934; SEQ ID NO: 281 and SEQ ID NO: 935; SEQ ID NO: 281 and SEQ ID NO: 936; SEQ ID NO: 281 and SEQ ID NO: 937; SEQ ID NO: 281 and SEQ ID NO: 938; SEQ ID NO: 282 and SEQ ID NO: 924; SEQ ID NO: 282 and SEQ ID NO: 925; SEQ ID NO: 282 and SEQ ID NO: 926; SEQ ID NO: 282 and SEQ ID NO: 927; SEQ ID NO: 282 and SEQ ID NO: 928; SEQ ID NO: 282 and SEQ ID NO: 929; SEQ ID NO: 282 and SEQ ID NO: 930; SEQ ID NO: 282 and SEQ ID NO: 931; SEQ ID NO: 282 and SEQ ID NO: 932; SEQ ID NO: 282 and SEQ ID NO: 933; SEQ ID NO: 282 and SEQ ID NO: 934; SEQ ID NO: 282 and SEQ ID NO: 935; SEQ ID NO: 282 and SEQ ID NO: 936; SEQ ID NO: 282 and SEQ ID NO: 937; SEQ ID NO: 282 and SEQ ID NO: 938; SEQ ID NO: 283 and SEQ ID NO: 924; SEQ ID NO: 283 and SEQ ID NO: 925; SEQ ID NO: 283 and SEQ ID NO: 926; SEQ ID NO: 283 and SEQ ID NO: 927; SEQ ID NO: 283 and SEQ ID NO: 928; SEQ ID NO: 283 and SEQ ID NO: 929; SEQ ID NO: 283 and SEQ ID NO: 930; SEQ ID NO: 283 and SEQ ID NO: 931; SEQ ID NO: 283 and SEQ ID NO: 932; SEQ ID NO: 283 and SEQ ID NO: 933; SEQ ID NO: 283 and SEQ ID NO: 934; SEQ ID NO: 283 and SEQ ID NO: 935; SEQ ID NO: 283 and SEQ ID NO: 936; SEQ ID NO: 283 and SEQ ID NO: 937; SEQ ID NO: 283 and SEQ ID NO: 938; SEQ ID NO: 284 and SEQ ID NO: 924; SEQ ID NO: 284 and SEQ ID NO: 925; SEQ ID NO: 284 and SEQ ID NO: 926; SEQ ID NO: 284 and SEQ ID NO: 927; SEQ ID NO: 284 and SEQ ID NO: 928; SEQ ID NO: 284 and SEQ ID NO: 929; SEQ ID NO: 284 and SEQ ID NO: 930; SEQ ID NO: 284 and SEQ ID NO: 931; SEQ ID NO: 284 and SEQ ID NO: 932; SEQ ID NO: 284 and SEQ ID NO: 933; SEQ ID NO: 284 and SEQ ID NO: 934; SEQ ID NO: 284 and SEQ ID NO: 935; SEQ ID NO: 284 and SEQ ID NO: 936; SEQ ID NO: 284 and SEQ ID NO: 937; SEQ ID NO: 284 and SEQ ID NO: 938; SEQ ID NO: 285 and SEQ ID NO: 924; SEQ ID NO: 285 and SEQ ID NO: 925; SEQ ID NO: 285 and SEQ ID NO: 926; SEQ ID NO: 285 and SEQ ID NO: 927; SEQ ID NO: 285 and SEQ ID NO: 928; SEQ ID NO: 285 and SEQ ID NO: 929; SEQ ID NO: 285 and SEQ ID NO: 930; SEQ ID NO: 285 and SEQ ID NO: 931; SEQ ID NO: 285 and SEQ ID NO: 932; SEQ ID NO: 285 and SEQ ID NO: 933; SEQ ID NO: 285 and SEQ ID NO: 934; SEQ ID NO: 285 and SEQ ID NO: 935; SEQ ID NO: 285 and SEQ ID NO: 936; SEQ ID NO: 285 and SEQ ID NO: 937; SEQ ID NO: 285 and SEQ ID NO: 938; SEQ ID NO: 286 and SEQ ID NO: 924; SEQ ID NO: 286 and SEQ ID NO: 925; SEQ ID NO: 286 and SEQ ID NO: 926; SEQ ID NO: 286 and SEQ ID NO: 927; SEQ ID NO: 286 and SEQ ID NO: 928; SEQ ID NO: 286 and SEQ ID NO: 929; SEQ ID NO: 286 and SEQ ID NO: 930; SEQ ID NO: 286 and SEQ ID NO: 931; SEQ ID NO: 286 and SEQ ID NO: 932; SEQ ID NO: 286 and SEQ ID NO: 933; SEQ ID NO: 286 and SEQ ID NO: 934; SEQ ID NO: 286 and SEQ ID NO: 935; SEQ ID NO: 286 and SEQ ID NO: 936; SEQ ID NO: 286 and SEQ ID NO: 937; SEQ ID NO: 286 and SEQ ID NO: 938; SEQ ID NO: 287 and SEQ ID NO: 924; SEQ ID NO: 287 and SEQ ID NO: 925; SEQ ID NO: 287 and SEQ ID NO: 926; SEQ ID NO: 287 and SEQ ID NO: 927; SEQ ID NO: 287 and SEQ ID NO: 928; SEQ ID NO: 287 and SEQ ID NO: 929; SEQ ID NO: 287 and SEQ ID NO: 930; SEQ ID NO: 287 and SEQ ID NO: 931; SEQ ID NO: 287 and SEQ ID NO: 932; SEQ ID NO: 287 and SEQ ID NO: 933; SEQ ID NO: 287 and SEQ ID NO: 934; SEQ ID NO: 287 and SEQ ID NO: 935; SEQ ID NO: 287 and SEQ ID NO: 936; SEQ ID NO: 287 and SEQ ID NO: 937; SEQ ID NO: 287 and SEQ ID NO: 938; SEQ ID NO: 288 and SEQ ID NO: 924; SEQ ID NO: 288 and SEQ ID NO: 925; SEQ ID NO: 288 and SEQ ID NO: 926; SEQ ID NO: 288 and SEQ ID NO: 927; SEQ ID NO: 288 and SEQ ID NO: 928; SEQ ID NO: 288 and SEQ ID NO: 929; SEQ ID NO: 288 and SEQ ID NO: 930; SEQ ID NO: 288 and SEQ ID NO: 931; SEQ ID NO: 288 and SEQ ID NO: 932; SEQ ID NO: 288 and SEQ ID NO: 933; SEQ ID NO: 288 and SEQ ID NO: 934; SEQ ID NO: 288 and SEQ ID NO: 935; SEQ ID NO: 288 and SEQ ID NO: 936; SEQ ID NO: 288 and SEQ ID NO: 937; SEQ ID NO: 288 and SEQ ID NO: 938; SEQ ID NO: 289 and SEQ ID NO: 924; SEQ ID NO: 289 and SEQ ID NO: 925; SEQ ID NO: 289 and SEQ ID NO: 926; SEQ ID NO: 289 and SEQ ID NO: 927; SEQ ID NO: 289 and SEQ ID NO: 928; SEQ ID NO: 289 and SEQ ID NO: 929; SEQ ID NO: 289 and SEQ ID NO: 930; SEQ ID NO: 289 and SEQ ID NO: 931; SEQ ID NO: 289 and SEQ ID NO: 932; SEQ ID NO: 289 and SEQ ID NO: 933; SEQ ID NO: 289 and SEQ ID NO: 934; SEQ ID NO: 289 and SEQ ID NO: 935; SEQ ID NO: 289 and SEQ ID NO: 936; SEQ ID NO: 289 and SEQ ID NO: 937; SEQ ID NO: 289 and SEQ ID NO: 938; SEQ ID NO: 290 and SEQ ID NO: 924; SEQ ID NO: 290 and SEQ ID NO: 925; SEQ ID NO: 290 and SEQ ID NO: 926; SEQ ID NO: 290 and SEQ ID NO: 927; SEQ ID NO: 290 and SEQ ID NO: 928; SEQ ID NO: 290 and SEQ ID NO: 929; SEQ ID NO: 290 and SEQ ID NO: 930; SEQ ID NO: 290 and SEQ ID NO: 931; SEQ ID NO: 290 and SEQ ID NO: 932; SEQ ID NO: 290 and SEQ ID NO: 933; SEQ ID NO: 290 and SEQ ID NO: 934; SEQ ID NO: 290 and SEQ ID NO: 935; SEQ ID NO: 290 and SEQ ID NO: 936; SEQ ID NO: 290 and SEQ ID NO: 937; SEQ ID NO: 290 and SEQ ID NO: 938; SEQ ID NO: 291 and SEQ ID NO: 924; SEQ ID NO: 291 and SEQ ID NO: 925; SEQ ID NO: 291 and SEQ ID NO: 926; SEQ ID NO: 291 and SEQ ID NO: 927; SEQ ID NO: 291 and SEQ ID NO: 928; SEQ ID NO: 291 and SEQ ID NO: 929; SEQ ID NO: 291 and SEQ ID NO: 930; SEQ ID NO: 291 and SEQ ID NO: 931; SEQ ID NO: 291 and SEQ ID NO: 932; SEQ ID NO: 291 and SEQ ID NO: 933; SEQ ID NO: 291 and SEQ ID NO: 934; SEQ ID NO: 291 and SEQ ID NO: 935; SEQ ID NO: 291 and SEQ ID NO: 936; SEQ ID NO: 291 and SEQ ID NO: 937; SEQ ID NO: 291 and SEQ ID NO: 938; SEQ ID NO: 292 and SEQ ID NO: 924; SEQ ID NO: 292 and SEQ ID NO: 925; SEQ ID NO: 292 and SEQ ID NO: 926; SEQ ID NO: 292 and SEQ ID NO: 927; SEQ ID NO: 292 and SEQ ID NO: 928; SEQ ID NO: 292 and SEQ ID NO: 929; SEQ ID NO: 292 and SEQ ID NO: 930; SEQ ID NO: 292 and SEQ ID NO: 931; SEQ ID NO: 292 and SEQ ID NO: 932; SEQ ID NO: 292 and SEQ ID NO: 933; SEQ ID NO: 292 and SEQ ID NO: 934; SEQ ID NO: 292 and SEQ ID NO: 935; SEQ ID NO: 292 and SEQ ID NO: 936; SEQ ID NO: 292 and SEQ ID NO: 937; SEQ ID NO: 292 and SEQ ID NO: 938; SEQ ID NO: 950 and SEQ ID NO: 275; SEQ ID NO: 951 and SEQ ID NO: 275; SEQ ID NO: 952 and SEQ ID NO: 275; SEQ ID NO: 953 and SEQ ID NO: 275; SEQ ID NO: 954 and SEQ ID NO: 275; or SEQ ID NO: 955 and SEQ ID NO: 275.
Embodiment 4 is a composition comprising:
   a. a single nucleic acid molecule comprising:
      i. a nucleic acid encoding Staphylococcus aureus Cas9 (SaCas9) or Staphylococcus lugdunensis (SluCas9) and at least one, at least two, or at least three guide RNAs; or
      ii. a nucleic acid encoding Staphylococcus aureus Cas9 (SaCas9) or Staphylococcus lugdunensis (SluCas9) and from one to n guide RNAs, wherein n is no more than the maximum number of guide RNAs that can be expressed from said nucleic acid; or
      iii. a nucleic acid encoding Staphylococcus aureus Cas9 (SaCas9) or Staphylococcus lugdunensis (SluCas9) and 1, 2, or 3 guide RNAs;
wherein each guide RNA is selected from Table 6, optionally wherein the composition comprises at least one pair of guide RNAs, wherein the at least one pair is selected from the pairs shown in Tables 1B, 1D, 3B, 3D, 5B, or 5D; or
   b. two nucleic acid molecules comprising:
      i. a first nucleic acid encoding Staphylococcus aureus Cas9 (SaCas9) or Staphylococcus lugdunensis (SluCas9); and
         1. a second nucleic acid that does not encode a SaCas9 or SluCas9 and encodes any one of the following:
         2. at least one, at least two, at least three, at least four, at least five, or at least six guide RNAs; or
         3. from one to n guide RNAs, wherein n is no more than the maximum number of guide RNAs that can be expressed from said nucleic acid; or
         4. from one to six guide RNAs; or
      ii. a first nucleic acid encoding Staphylococcus aureus Cas9 (SaCas9) or Staphylococcus lugdunensis (SluCas9) and
         1. at least one, at least two, or at least three guide RNAs; or
         2. from one to n guide RNAs, wherein n is no more than the maximum number of guide RNAs that can be expressed from said nucleic acid; or
         3. 1, 2, or 3 guide RNAs; and
      a second nucleic acid that does not encode a SaCas9 or SluCas9, optionally wherein the second nucleic acid comprises any one of the following:
         1. at least one, at least two, at least three, at least four, at least five, or at least six guide RNAs; or
         2. from one to n guide RNAs, wherein n is no more than the maximum number of guide RNAs that can be expressed from said nucleic acid; or
         3. from one to six guide RNAs; or
      iii. a first nucleic acid encoding Staphylococcus aureus Cas9 (SaCas9) or Staphylococcus lugdunensis (SluCas9) and at least one, at least two, or at least three guide RNAs; and a second nucleic acid that does not encode a SaCas9 or SluCas9 and encodes from one to six guide RNAs; or
      iv. a first nucleic acid encoding Staphylococcus aureus Cas9 (SaCas9) or Staphylococcus lugdunensis (SluCas9) and at least two guide RNAs, wherein a first guide RNA of the at least two guide RNAs binds upstream of a target sequence and a second guide RNA of the at least two guide RNAs binds downstream of the target sequence; and
      a second nucleic acid that does not encode a SaCas9 or SluCas9 and encodes at least one additional copy of each of the guide RNAs encoded in the first nucleic acid;
wherein each guide RNA is selected from Table 6, optionally wherein the composition comprises at least one pair of guide RNAs, wherein the at least one pair is selected from the pairs shown in Tables 1B, 1D, 3B, 3D, 5B, or 5D.
Embodiment 5 is a composition of any one of embodiments 1-4, comprising a pair of guide RNAs, wherein the pair of guide RNAs is capable of excising a DNA fragment from the DMD gene; wherein the DNA fragment is between 5-250 nucleotides in length.
Embodiment 6 is a composition of embodiment 5, wherein the excised DNA fragment does not comprise an entire exon of the DMD gene.
Embodiment 7 is a composition comprising a single nucleic acid molecule encoding a pair of guide RNAs and a Cas9, wherein the single nucleic acid molecule comprises:
   a. a first nucleic acid encoding the pair of guide RNAs, wherein the pair of guide RNAs comprises any one of the following pairs of guide sequences: SEQ ID NOs: 1 and 3; 16 and 17; 16 and 18; 16 and 19; 16 and 20; 16 and 21; 16 and 22; 16 and 23; 16 and 24; 16 and 25; 16 and 26; 17 and 18; 17 and 19; 17 and 20; 17 and 21; 17 and 22; 17 and 23; 17 and 24; 17 and 25; 17 and 26; 18 and 19; 18 and 20; 18 and 21; 18 and 22; 18 and 23; 18 and 24; 18 and 25; 18 and 26; 19 and 20; 19 and 21; 19 and 22; 19 and 23; 19 and 24; 19 and 25; 19 and 26; 20 and 21; 20 and 22; 20 and 23; 20 and 24; 20 and 25; 20 and 26; 21 and 22; 21 and 23; 21 and 24; 21 and 25; 21 and 26; 22 and 23; 22 and 24; 22 and 25; 22 and 26; 23 and 24; 23 and 25; 23 and 26; 24 and 25; 24 and 26; 25 and 26; 110 and 120; 110 and 121; 110 and 122; 110 and 123; 110 and 124; 110 and 125; 111 and 112; 111 and 113; 111 and 114; 111 and 115; 111 and 116; 111 and 117; 111 and 118; 111 and 119; 111 and 120; 111 and 121; 111 and 122; 111 and 123; 111 and 124; 111 and 125; 112 and 113; 112 and 114; 112 and 115; 112 and 116; 112 and 117; 112 and 118; 112 and 119; 112 and 120; 112 and 121; 112 and 122; 112 and 123; 112 and 124; 112 and 125; 113 and 114; 113 and 115; 113 and 116; 113 and 117; 113 and 118; 113 and 119; 113 and 120; 113 and 121; 113 and 122; 113 and 123; 113 and 124; 113 and 125; 114 and 115; 114 and 116; 114 and 117; 114 and 118; 114 and 119; 114 and 120; 114 and 121; 114 and 122; 114 and 123; 114 and 124; 114 and 125; 115 and 116; 115 and 117; 115 and 118; 115 and 119; 115 and 120; 115 and 121; 115 and 122; 115 and 123; 115 and 124; 115 and 125; 116 and 117; 116 and 118; 116 and 119; 116 and 120; 116 and 121; 116 and 122; 116 and 123; 116 and 124; 116 and 125; 117 and 118; 117 and 119; 117 and 120; 117 and 121; 117 and 122; 117 and 123; 117 and 124; 117 and 125; 118 and 119; 118 and 120; 118 and 121; 118 and 122; 118 and 123; 118 and 124; 118 and 125; 119 and 120; 119 and 121; 119 and 122; 119 and 123; 119 and 124; 119 and 125; 120 and 121; 120 and 122; 120 and 123; 120 and 124; 120 and 125; 121 and 122; 121 and 123; 121 and 124; 121 and 125; 122 and 123; 122 and 124; 122 and 125; 123 and 124; 123 and 125; 124 and 125; 148 and 149; 148 and 150; 148 and 151; 148 and 152; 148 and 153; 148 and 154; 148 and 155; 148 and 156; 148 and 157; 148 and 158; 148 and 159; 149 and 150; 149 and 151; 149 and 152; 149 and 153; 149 and 154; 149 and 155; 149 and 156; 149 and 157; 149 and 158; 149 and 159; 150 and 151; 150 and 152; 150 and 153; 150 and 154; 150 and 155; 150 and 156; 150 and 157; 150 and 158; 150 and 159; 151 and 152; 151 and 153; 151 and 154; 151 and 155; 151 and 156; 151 and 157; 151 and 158; 151 and 159; 152 and 153; 152 and 154; 152 and 155; 152 and 156; 152 and 157; 152 and 158; 152 and 159; 153 and 154; 153 and 155; 153 and 156; 153 and 157; 153 and 158; 153 and 159; 154 and 155; 154 and 156; 154 and 157; 154 and 158; 154 and 159; 155 and 156; 155 and 157; 155 and 158; 155 and 159; 156 and 157; 156 and 158; 156 and 159; 157 and 158; 157 and 159; 158 and 159; 71 and 72; 71 and 73; 71 and 74; 71 and 75; 71 and 76; 71 and 77; 71 and 78; 71 and 84; 71 and 85; 71 and 86; 71 and 87; 71 and 88; 71 and 89; 71 and 90; 71 and 91; 71 and 92; 71 and 93; 71 and 94; 71 and 95; 71 and 96; 71 and 97; 71 and 98; 71 and 99; 71 and 100; 71 and 101; 71 and 102; 72 and 73; 72 and 74; 72 and 75; 72 and 76; 72 and 77; 72 and 78; 72 and 84; 72 and 85; 72 and 86; 72 and 87; 72 and 88; 72 and 89; 72 and 90; 72 and 91; 72 and 92; 72 and 93; 72 and 94; 72 and 95; 72 and 96; 72 and 97; 72 and 98; 72 and 99; 72 and 100; 72 and 101; 72 and 102; 73 and 74; 73 and 75; 73 and 76; 73 and 77; 73 and 78; 73 and 84; 73 and 85; 73 and 86; 73 and 87; 73 and 88; 73 and 89; 73 and 90; 73 and 91; 73 and 92; 73 and 93; 73 and 94; 73 and 95; 73 and 96; 73 and 97; 73 and 98; 73 and 99; 73 and 100; 73 and 101; 73 and 102; 74 and 75; 74 and 76; 74 and 77; 74 and 78; 74 and 84; 74 and 85; 74 and 86; 74 and 87; 74 and 88; 74 and 89; 74 and 90; 74 and 91; 74 and 92; 74 and 93; 74 and 94; 74 and 95; 74 and 96; 74 and 97; 74 and 98; 74 and 99; 74 and 100; 74 and 101; 74 and 102; 75 and 76; 75 and 77; 75 and 78; 75 and 84; 75 and 85; 75 and 86; 75 and 87; 75 and 88; 75 and 89; 75 and 90; 75 and 91; 75 and 92; 75 and 93; 75 and 94; 75 and 95; 75 and 96; 75 and 97; 75 and 98; 75 and 99; 75 and 100; 75 and 101; 75 and 102; 76 and 77; 76 and 78; 76 and 84; 76 and 85; 76 and 86; 76 and 87; 76 and 88; 76 and 89; 76 and 90; 76 and 91; 76 and 92; 76 and 93; 76 and 94; 76 and 95; 76 and 96; 76 and 97; 76 and 98; 76 and 99; 76 and 100; 76 and 101; 76 and 102; 77 and 78; 77 and 84; 77 and 85; 77 and 86; 77 and 87; 77 and 88; 77 and 89; 77 and 90; 77 and 91; 77 and 92; 77 and 93; 77 and 94; 77 and 95; 77 and 96; 77 and 97; 77 and 98; 77 and 99; 77 and 100; 77 and 101; 77 and 102; 78 and 84; 78 and 85; 78 and 86; 78 and 87; 78 and 88; 78 and 89; 78 and 90; 78 and 91; 78 and 92; 78 and 93; 78 and 94; 78 and 95; 78 and 96; 78 and 97; 78 and 98; 78 and 99; 78 and 100; 78 and 101; 78 and 102; 84 and 85; 84 and 86; 84 and 87; 84 and 88; 84 and 89; 84 and 90; 84 and 91; 84 and 92; 84 and 93; 84 and 94; 84 and 95; 84 and 96; 84 and 97; 84 and 98; 84 and 99; 84 and 100; 84 and 101; 84 and 102; 85 and 86; 85 and 87; 85 and 88; 85 and 89; 85 and 90; 85 and 91; 85 and 92; 85 and 93; 85 and 94; 85 and 95; 85 and 96; 85 and 97; 85 and 98; 85 and 99; 85 and 100; 85 and 101; 85 and 102; 86 and 87; 86 and 88; 86 and 89; 86 and 90; 86 and 91; 86 and 92; 86 and 93; 86 and 94; 86 and 95; 86 and 96; 86 and 97; 86 and 98; 86 and 99; 86 and 100; 86 and 101; 86 and 102; 87 and 88; 87 and 89; 87 and 90; 87 and 91; 87 and 92; 87 and 93; 87 and 94; 87 and 95; 87 and 96; 87 and 97; 87 and 98; 87 and 99; 87 and 100; 87 and 101; 87 and 102; 88 and 89; 88 and 90; 88 and 91; 88 and 92; 88 and 93; 88 and 94; 88 and 95; 88 and 96; 88 and 97; 88 and 98; 88 and 99; 88 and 100; 88 and 101; 88 and 102; 89 and 90; 89 and 91; 89 and 92; 89 and 93; 89 and 94; 89 and 95; 89 and 96; 89 and 97; 89 and 98; 89 and 99; 89 and 100; 89 and 101; 89 and 102; 90 and 91; 90 and 92; 90 and 93; 90 and 94; 90 and 95; 90 and 96; 90 and 97; 90 and 98; 90 and 99; 90 and 100; 90 and 101; 90 and 102; 91 and 92; 91 and 93; 91 and 94; 91 and 95; 91 and 96; 91 and 97; 91 and 98; 91 and 99; 91 and 100; 91 and 101; 91 and 102; 92 and 93; 92 and 94; 92 and 95; 92 and 96; 92 and 97; 92 and 98; 92 and 99; 92 and 100; 92 and 101; 92 and 102; 93 and 94; 93 and 95; 93 and 96; 93 and 97; 93 and 98; 93 and 99; 93 and 100; 93 and 101; 93 and 102; 94 and 95; 94 and 96; 94 and 97; 94 and 98; 94 and 99; 94 and 100; 94 and 101; 94 and 102; 95 and 96; 95 and 97; 95 and 98; 95 and 99; 95 and 100; 95 and 101; 95 and 102; 96 and 97; 96 and 98; 96 and 99; 96 and 100; 96 and 101; 96 and 102; 97 and 98; 97 and 99; 97 and 100; 97 and 101; 97 and 102; 98 and 99; 98 and 100; 98 and 101; 98 and 102; 99 and 100; 99 and 101; 99 and 102; 100 and 101; 100 and 102; or 101 and 102; and a second nucleic acid encoding a Staphylococcus aureus Cas9 (SaCas9); or
   b. a first nucleic acid encoding a pair of guide RNAs, wherein the pair of guide RNAs comprises any one of the following pairs of guide sequences: SEQ ID NOs: 200 and 201; 200 and 202; 200 and 203; 200 and 204; 200 and 205; 200 and 206; 200 and 207; 200 and 208; 201 and 202; 201 and 203; 201 and 204; 201 and 205; 201 and 206; 201 and 207; 201 and 208; 202 and 203; 202 and 204; 202 and 205; 202 and 206; 202 and 207; 202 and 208; 203 and 204; 203 and 205; 203 and 206; 203 and 207; 203 and 208; 204 and 205; 204 and 206; 204 and 207; 204 and 208; 205 and 206; 205 and 207; 205 and 208; 206 and 207; 206 and 208; 207 and 208; 231 and 232; 231 and 233; 231 and 234; 231 and 235; 231 and 236; 231 and 237; 231 and 238; 231 and 239; 231 and 240; 231 and 241; 231 and 242; 232 and 233; 232 and 234; 232 and 235; 232 and 236; 232 and 237; 232 and 238; 232 and 239; 232 and 240; 232 and 241; 232 and 242; 233 and 234; 233 and 235; 233 and 236; 233 and 237; 233 and 238; 233 and 239; 233 and 240; 233 and 241; 233 and 242; 234 and 235; 234 and 236; 234 and 237; 234 and 238; 234 and 239; 234 and 240; 234 and 241; 234 and 242; 235 and 236; 235 and 237; 235 and 238; 235 and 239; 235 and 240; 235 and 241; 235 and 242; 236 and 237; 236 and 238; 236 and 239; 236 and 240; 236 and 241; 236 and 242; 237 and 238; 237 and 239; 237 and 240; 237 and 241; 237 and 242; 238 and 239; 238 and 240; 238 and 241; 238 and 242; 239 and 240; 239 and 241; 239 and 242; 240 and 241; 240 and 242; 241 and 242; 270 and 271; 270 and 272; 270 and 273; 270 and 274; 270 and 275; 270 and 276; 270 and 277; 270 and 278; 270 and 279; 270 and 280; 270 and 281; 270 and 282; 270 and 283; 270 and 284; 270 and 285; 270 and 286; 270 and 287; 270 and 288; 270 and 289; 270 and 290; 270 and 291; 270 and 292; 271 and 272; 271 and 273; 271 and 274; 271 and 275; 271 and 276; 271 and 277; 271 and 278; 271 and 279; 271 and 280; 271 and 281; 271 and 282; 271 and 283; 271 and 284; 271 and 285; 271 and 286; 271 and 287; 271 and 288; 271 and 289; 271 and 290; 271 and 291; 271 and 292; 272 and 273; 272 and 274; 272 and 275; 272 and 276; 272 and 277; 272 and 278; 272 and 279; 272 and 280; 272 and 281; 272 and 282; 272 and 283; 272 and 284; 272 and 285; 272 and 286; 272 and 287; 272 and 288; 272 and 289; 272 and 290; 272 and 291; 272 and 292; 273 and 274; 273 and 275; 273 and 276; 273 and 277; 273 and 278; 273 and 279; 273 and 280; 273 and 281; 273 and 282; 273 and 283; 273 and 284; 273 and 285; 273 and 286; 273 and 287; 273 and 288; 273 and 289; 273 and 290; 273 and 291; 273 and 292; 274 and 275; 274 and 276; 274 and 277; 274 and 278; 274 and 279; 274 and 280; 274 and 281; 274 and 282; 274 and 283; 274 and 284; 274 and 285; 274 and 286; 274 and 287; 274 and 288; 274 and 289; 274 and 290; 274 and 291; 274 and 292; 275 and 276; 275 and 277; 275 and 278; 275 and 279; 275 and 280; 275 and 281; 275 and 282; 275 and 283; 275 and 284; 275 and 285; 275 and 286; 275 and 287; 275 and 288; 275 and 289; 275 and 290; 275 and 291; 275 and 292; 276 and 277; 276 and 278; 276 and 279; 276 and 280; 276 and 281; 276 and 282; 276 and 283; 276 and 284; 276 and 285; 276 and 286; 276 and 287; 276 and 288; 276 and 289; 276 and 290; 276 and 291; 276 and 292; 277 and 278; 277 and 279; 277 and 280; 277 and 281; 277 and 282; 277 and 283; 277 and 284; 277 and 285; 277 and 286; 277 and 287; 277 and 288; 277 and 289; 277 and 290; 277 and 291; 277 and 292; 278 and 279; 278 and 280; 278 and 281; 278 and 282; 278 and 283; 278 and 284; 278 and 285; 278 and 286; 278 and 287; 278 and 288; 278 and 289; 278 and 290; 278 and 291; 278 and 292; 279 and 280; 279 and 281; 279 and 282; 279 and 283; 279 and 284; 279 and 285; 279 and 286; 279 and 287; 279 and 288; 279 and 289; 279 and 290; 279 and 291; 279 and 292; 280 and 281; 280 and 282; 280 and 283; 280 and 284; 280 and 285; 280 and 286; 280 and 287; 280 and 288; 280 and 289; 280 and 290; 280 and 291; 280 and 292; 281 and 282; 281 and 283; 281 and 284; 281 and 285; 281 and 286; 281 and 287; 281 and 288; 281 and 289; 281 and 290; 281 and 291; 281 and 292; 282 and 283; 282 and 284; 282 and 285; 282 and 286; 282 and 287; 282 and 288; 282 and 289; 282 and 290; 282 and 291; 282 and 292; 283 and 284; 283 and 285; 283 and 286; 283 and 287; 283 and 288; 283 and 289; 283 and 290; 283 and 291; 283 and 292; 284 and 285; 284 and 286; 284 and 287; 284 and 288; 284 and 289; 284 and 290; 284 and 291; 284 and 292; 285 and 286; 285 and 287; 285 and 288; 285 and 289; 285 and 290; 285 and 291; 285 and 292; 286 and 287; 286 and 288; 286 and 289; 286 and 290; 286 and 291; 286 and 292; 287 and 288; 287 and 289; 287 and 290; 287 and 291; 287 and 292; 288 and 289; 288 and 290; 288 and 291; 288 and 292; 289 and 290; 289 and 291; 289 and 292; 290 and 291; 290 and 292; 291 and 292; 924 and 270; 924 and 272; 924 and 273; 924 and 274; 924 and 275; 924 and 276; 924 and 277; 924 and 278; 924 and 279; 924 and 280; 924 and 281; 924 and 282; 924 and 283; 924 and 284; 924 and 285; 924 and 286; 924 and 287; 924 and 288; 924 and 289; 924 and 290; 924 and 291; 924 and 292; 925 and 270; 925 and 272; 925 and 273; 925 and 274; 925 and 275; 925 and 276; 925 and 277; 925 and 278; 925 and 279; 925 and 280; 925 and 281; 925 and 282; 925 and 283; 925 and 284; 925 and 285; 925 and 286; 925 and 287; 925 and 288; 925 and 289; 925 and 290; 925 and 291; 925 and 292; 926 and 270; 926 and 272; 926 and 273; 926 and 274; 926 and 275; 926 and 276; 926 and 277; 926 and 278; 926 and 279; 926 and 280; 926 and 281; 926 and 282; 926 and 283; 926 and 284; 926 and 285; 926 and 286; 926 and 287; 926 and 288; 926 and 289; 926 and 290; 926 and 291; 926 and 292; 927 and 270; 927 and 272; 927 and 273; 927 and 274; 927 and 275; 927 and 276; 927 and 277; 927 and 278; 927 and 279; 927 and 280; 927 and 281; 927 and 282; 927 and 283; 927 and 284; 927 and 285; 927 and 286; 927 and 287; 927 and 288; 927 and 289; 927 and 290; 927 and 291; 927 and 292; 928 and 270; 928 and 272; 928 and 273; 928 and 274; 928 and 275; 928 and 276; 928 and 277; 928 and 278; 928 and 279; 928 and 280; 928 and 281; 928 and 282; 928 and 283; 928 and 284; 928 and 285; 928 and 286; 928 and 287; 928 and 288; 928 and 289; 928 and 290; 928 and 291; 928 and 292; 929 and 270; 929 and 272; 929 and 273; 929 and 274; 929 and 275; 929 and 276; 929 and 277; 929 and 278; 929 and 279; 929 and 280; 929 and 281; 929 and 282; 929 and 283; 929 and 284; 929 and 285; 929 and 286; 929 and 287; 929 and 288; 929 and 289; 929 and 290; 929 and 291; 929 and 292; 930 and 270; 930 and 272; 930 and 273; 930 and 274; 930 and 275; 930 and 276; 930 and 277; 930 and 278; 930 and 279; 930 and 280; 930 and 281; 930 and 282; 930 and 283; 930 and 284; 930 and 285; 930 and 286; 930 and 287; 930 and 288; 930 and 289; 930 and 290; 930 and 291; 930 and 292; 931 and 270; 931 and 272; 931 and 273; 931 and 274; 931 and 275; 931 and 276; 931 and 277; 931 and 278; 931 and 279; 931 and 280; 931 and 281; 931 and 282; 931 and 283; 931 and 284; 931 and 285; 931 and 286; 931 and 287; 931 and 288; 931 and 289; 931 and 290; 931 and 291; 931 and 292; 932 and 270; 932 and 272; 932 and 273; 932 and 274; 932 and 275; 932 and 276; 932 and 277; 932 and 278; 932 and 279; 932 and 280; 932 and 281; 932 and 282; 932 and 283; 932 and 284; 932 and 285; 932 and 286; 932 and 287; 932 and 288; 932 and 289; 932 and 290; 932 and 291; 932 and 292; 933 and 270; 933 and 272; 933 and 273; 933 and 274; 933 and 275; 933 and 276; 933 and 277; 933 and 278; 933 and 279; 933 and 280; 933 and 281; 933 and 282; 933 and 283; 933 and 284; 933 and 285; 933 and 286; 933 and 287; 933 and 288; 933 and 289; 933 and 290; 933 and 291; 933 and 292; 934 and 270; 934 and 272; 934 and 273; 934 and 274; 934 and 275; 934 and 276; 934 and 277; 934 and 278; 934 and 279; 934 and 280; 934 and 281; 934 and 282; 934 and 283; 934 and 284; 934 and 285; 934 and 286; 934 and 287; 934 and 288; 934 and 289; 934 and 290; 934 and 291; 934 and 292; 935 and 270; 935 and 272; 935 and 273; 935 and 274; 935 and 275; 935 and 276; 935 and 277; 935 and 278; 935 and 279; 935 and 280; 935 and 281; 935 and 282; 935 and 283; 935 and 284; 935 and 285; 935 and 286; 935 and 287; 935 and 288; 935 and 289; 935 and 290; 935 and 291; 935 and 292; 936 and 270; 936 and 272; 936 and 273; 936 and 274; 936 and 275; 936 and 276; 936 and 277; 936 and 278; 936 and 279; 936 and 280; 936 and 281; 936 and 282; 936 and 283; 936 and 284; 936 and 285; 936 and 286; 936 and 287; 936 and 288; 936 and 289; 936 and 290; 936 and 291; 936 and 292; 937 and 270; 937 and 272; 937 and 273; 937 and 274; 937 and 275; 937 and 276; 937 and 277; 937 and 278; 937 and 279; 937 and 280; 937 and 281; 937 and 282; 937 and 283; 937 and 284; 937 and 285; 937 and 286; 937 and 287; 937 and 288; 937 and 289; 937 and 290; 937 and 291; 937 and 292; 938 and 270; 938 and 272; 938 and 273; 938 and 274; 938 and 275; 938 and 276; 938 and 277; 938 and 278; 938 and 279; 938 and 280; 938 and 281; 938 and 282; 938 and 283; 938 and 284; 938 and 285; 938 and 286; 938 and 287; 938 and 288; 938 and 289; 938 and 290; 938 and 291; 938 and 292; 950 and 275; 951 and 275; 952 and 275; 953 and 275; 954 and 275; or 955 and 275; and a second nucleic acid encoding a Staphylococcus lugdunensis (SluCas9); or
   c. a first nucleic acid encoding a pair of guide RNAs, wherein the pair of guide RNAs comprises any one of the following pairs of guide sequences targeting exon 44: SEQ ID NOs: 200 and 203; 200 and 204; 202 and 203; 202 and 205; 202 and 206; 202 and 207; 204 and 208; 204 and 205; 204 and 206; 204 and 207; or 204 and 208; and a second nucleic acid encoding a Staphylococcus lugdunensis (SluCas9); or
   d. a first nucleic acid encoding a pair of guide RNAs, wherein the pair of guide RNAs comprises any one of the following pairs of guide sequences targeting exon 50: SEQ ID NOs: 231 and 232; 231 and 234; 231 and 236; 231 and 237; 236 and 233; 236 and 235; 236 and 238; 236 and 240; or 236 and 241; and a second nucleic acid encoding a Staphylococcus lugdunensis (SluCas9); or
   e. a first nucleic acid encoding a pair of guide RNAs, wherein the pair of guide RNAs comprises any one of the following pairs of guide sequences targeting exon 53: SEQ ID NOs: 86 and 96; 87 and 96; 88 and 97; 89 and 96; 90 and 97; 92 and 77; 92 and 78; 92 and 96; 92 and 99; 93 and 98; 93 and 102; 94 and 100; 95 and 77; 95 and 78; 95 and 99; 96 and 100; 97 and 98; 97 and 102; 98 and 73; or 102 and 73; and a second nucleic acid encoding a Staphylococcus aureus Cas9 (SaCas9); or
   f. a first nucleic acid encoding a pair of guide RNAs, wherein the pair of guide RNAs comprises any one of the following pairs of guide sequences targeting exon 53: SEQ ID NOs: 278 and 290; 278 and 292; 281 and 291; 283 and 290; 283 and 292; 287 and 291; 272 and 290; 290 and 291; or 272 and 292; and a second nucleic acid encoding a Staphylococcus lugdunensis (SluCas9).
Embodiment 8 is a composition comprising one or more nucleic acid molecules encoding a Staphylococcus aureus Cas9 (SaCas9) and a first and a second guide RNA, wherein the first and the second guide RNAs target different sequences in a DMD gene, and wherein the first and the second guide RNAs each comprise a sequence that is at least 90% identical to a first and a second guide sequence selected from any one of the following pairs of first and second guide sequences:
any one of the following pairs of first and second guide sequences targeting exon 53, SEQ ID NOs: 86 and 96; 87 and 96; 88 and 97; 89 and 96; 90 and 97; 92 and 77; 92 and 78; 92 and 96; 92 and 99; 93 and 98; 93 and 102; 94 and 100; 95 and 77; 95 and 78; 95 and 99; 96 and 100; 97 and 98; 97 and 102; 98 and 73; or 102 and 73.
Embodiment 9 is a composition comprising one or more nucleic acid molecules encoding a Staphylococcus lugdunensis (SluCas9) and a first and a second guide RNA, wherein the first and the second guide RNAs target different sequences in a DMD gene, and wherein the first and the second guide RNAs each comprise a sequence that is at least 90% identical to a first and second guide sequence selected from any one of the following pairs of first and second guide sequences:
   a. any one of the following pairs of first and second guide sequences targeting exon 44, SEQ ID NOs: 200 and 203; 200 and 204; 202 and 203; 202 and 205; 202 and 206; 202 and 207; 204 and 208; 204 and 205; 204 and 206; 204 and 207; or 204 and 208;
   b. any one of the following pairs of first and second guide sequences targeting exon 50, SEQ ID NOs: 231 and 232; 231 and 234; 231 and 236; 231 and 237; 236 and 233; 236 and 235; 236 and 238; 236 and 240; or 236 and 241;
   c. any one of the following pairs of first and second guide sequences targeting exon 53, SEQ ID NOs: 278 and 290; 278 and 292; 281 and 291; 283 and 290; 283 and 292; 287 and 291; 272 and 290; 290 and 291; or 272 and 292.
Embodiment 10 is a composition comprising one or more nucleic acid molecules encoding an endonuclease and at least two guide RNAs, wherein the at least two guide RNAs each target a different sequence in a DMD gene, and wherein the at least two guide RNAs each comprise a sequence that is at least 90% identical to a first and second guide sequence selected from any one of the following pairs of first and second guide sequences:
   a. for exon 44, SEQ ID NOs: 1 and 3; 110 and 120; 110 and 121; 110 and 122; 110 and 123; 110 and 124; 110 and 125; 111 and 112; 111 and 113; 111 and 114; 111 and 115; 111 and 116; 111 and 117; 111 and 118; 111 and 119; 111 and 120; 111 and 121; 111 and 122; 111 and 123; 111 and 124; 111 and 125; 112 and 113; 112 and 114; 112 and 115; 112 and 116; 112 and 117; 112 and 118; 112 and 119; 112 and 120; 112 and 121; 112 and 122; 112 and 123; 112 and 124; 112 and 125; 113 and 114; 113 and 115; 113 and 116; 113 and 117; 113 and 118; 113 and 119; 113 and 120; 113 and 121; 113 and 122; 113 and 123; 113 and 124; 113 and 125; 114 and 115; 114 and 116; 114 and 117; 114 and 118; 114 and 119; 114 and 120; 114 and 121; 114 and 122; 114 and 123; 114 and 124; 114 and 125; 115 and 116; 115 and 117; 115 and 118; 115 and 119; 115 and 120; 115 and 121; 115 and 122; 115 and 123; 115 and 124; 115 and 125; 116 and 117; 116 and 118; 116 and 119; 116 and 120; 116 and 121; 116 and 122; 116 and 123; 116 and 124; 116 and 125; 117 and 118; 117 and 119; 117 and 120; 117 and 121; 117 and 122; 117 and 123; 117 and 124; 117 and 125; 118 and 119; 118 and 120; 118 and 121; 118 and 122; 118 and 123; 118 and 124; 118 and 125; 119 and 120; 119 and 121; 119 and 122; 119 and 123; 119 and 124; 119 and 125; 120 and 121; 120 and 122; 120 and 123; 120 and 124; 120 and 125; 121 and 122; 121 and 123; 121 and 124; 121 and 125; 122 and 123; 122 and 124; 122 and 125; 123 and 124; 123 and 125; or 124 and 125;
   b. for exon 50, SEQ ID NOs: 148 and 149; 148 and 150; 148 and 151; 148 and 152; 148 and 153; 148 and 154; 148 and 155; 148 and 156; 148 and 157; 148 and 158; 148 and 159; 149 and 150; 149 and 151; 149 and 152; 149 and 153; 149 and 154; 149 and 155; 149 and 156; 149 and 157; 149 and 158; 149 and 159; 150 and 151; 150 and 152; 150 and 153; 150 and 154; 150 and 155; 150 and 156; 150 and 157; 150 and 158; 150 and 159; 151 and 152; 151 and 153; 151 and 154; 151 and 155; 151 and 156; 151 and 157; 151 and 158; 151 and 159; 152 and 153; 152 and 154; 152 and 155; 152 and 156; 152 and 157; 152 and 158; 152 and 159; 153 and 154; 153 and 155; 153 and 156; 153 and 157; 153 and 158; 153 and 159; 154 and 155; 154 and 156; 154 and 157; 154 and 158; 154 and 159; 155 and 156; 155 and 157; 155 and 158; 155 and 159; 156 and 157; 156 and 158; 156 and 159; 157 and 158; 157 and 159; or 158 and 159;
   c. for exon 53, SEQ ID NOs: 16 and 17; 16 and 18; 16 and 19; 16 and 20; 16 and 21; 16 and 22; 16 and 23; 16 and 24; 16 and 25; 16 and 26; 17 and 18; 17 and 19; 17 and 20; 17 and 21; 17 and 22; 17 and 23; 17 and 24; 17 and 25; 17 and 26; 18 and 19; 18 and 20; 18 and 21; 18 and 22; 18 and 23; 18 and 24; 18 and 25; 18 and 26; 19 and 20; 19 and 21; 19 and 22; 19 and 23; 19 and 24; 19 and 25; 19 and 26; 20 and 21; 20 and 22; 20 and 23; 20 and 24; 20 and 25; 20 and 26; 21 and 22; 21 and 23; 21 and 24; 21 and 25; 21 and 26; 22 and 23; 22 and 24; 22 and 25; 22 and 26; 23 and 24; 23 and 25; 23 and 26; 24 and 25; 24 and 26; 25 and 26; 71 and 72; 71 and 73; 71 and 74; 71 and 75; 71 and 76; 71 and 77; 71 and 78; 71 and 84; 71 and 85; 71 and 86; 71 and 87; 71 and 88; 71 and 89; 71 and 90; 71 and 91; 71 and 92; 71 and 93; 71 and 94; 71 and 95; 71 and 96; 71 and 97; 71 and 98; 71 and 99; 71 and 100; 71 and 101; 71 and 102; 72 and 73; 72 and 74; 72 and 75; 72 and 76; 72 and 77; 72 and 78; 72 and 84; 72 and 85; 72 and 86; 72 and 87; 72 and 88; 72 and 89; 72 and 90; 72 and 91; 72 and 92; 72 and 93; 72 and 94; 72 and 95; 72 and 96; 72 and 97; 72 and 98; 72 and 99; 72 and 100; 72 and 101; 72 and 102; 73 and 74; 73 and 75; 73 and 76; 73 and 77; 73 and 78; 73 and 84; 73 and 85; 73 and 86; 73 and 87; 73 and 88; 73 and 89; 73 and 90; 73 and 91; 73 and 92; 73 and 93; 73 and 94; 73 and 95; 73 and 96; 73 and 97; 73 and 98; 73 and 99; 73 and 100; 73 and 101; 73 and 102; 74 and 75; 74 and 76; 74 and 77; 74 and 78; 74 and 84; 74 and 85; 74 and 86; 74 and 87; 74 and 88; 74 and 89; 74 and 90; 74 and 91; 74 and 92; 74 and 93; 74 and 94; 74 and 95; 74 and 96; 74 and 97; 74 and 98; 74 and 99; 74 and 100; 74 and 101; 74 and 102; 75 and 76; 75 and 77; 75 and 78; 75 and 84; 75 and 85; 75 and 86; 75 and 87; 75 and 88; 75 and 89; 75 and 90; 75 and 91; 75 and 92; 75 and 93; 75 and 94; 75 and 95; 75 and 96; 75 and 97; 75 and 98; 75 and 99; 75 and 100; 75 and 101; 75 and 102; 76 and 77; 76 and 78; 76 and 84; 76 and 85; 76 and 86; 76 and 87; 76 and 88; 76 and 89; 76 and 90; 76 and 91; 76 and 92; 76 and 93; 76 and 94; 76 and 95; 76 and 96; 76 and 97; 76 and 98; 76 and 99; 76 and 100; 76 and 101; 76 and 102; 77 and 78; 77 and 84; 77 and 85; 77 and 86; 77 and 87; 77 and 88; 77 and 89; 77 and 90; 77 and 91; 77 and 92; 77 and 93; 77 and 94; 77 and 95; 77 and 96; 77 and 97; 77 and 98; 77 and 99; 77 and 100; 77 and 101; 77 and 102; 78 and 84; 78 and 85; 78 and 86; 78 and 87; 78 and 88; 78 and 89; 78 and 90; 78 and 91; 78 and 92; 78 and 93; 78 and 94; 78 and 95; 78 and 96; 78 and 97; 78 and 98; 78 and 99; 78 and 100; 78 and 101; 78 and 102; 84 and 85; 84 and 86; 84 and 87; 84 and 88; 84 and 89; 84 and 90; 84 and 91; 84 and 92; 84 and 93; 84 and 94; 84 and 95; 84 and 96; 84 and 97; 84 and 98; 84 and 99; 84 and 100; 84 and 101; 84 and 102; 85 and 86; 85 and 87; 85 and 88; 85 and 89; 85 and 90; 85 and 91; 85 and 92; 85 and 93; 85 and 94; 85 and 95; 85 and 96; 85 and 97; 85 and 98; 85 and 99; 85 and 100; 85 and 101; 85 and 102; 86 and 87; 86 and 88; 86 and 89; 86 and 90; 86 and 91; 86 and 92; 86 and 93; 86 and 94; 86 and 95; 86 and 96; 86 and 97; 86 and 98; 86 and 99; 86 and 100; 86 and 101; 86 and 102; 87 and 88; 87 and 89; 87 and 90; 87 and 91; 87 and 92; 87 and 93; 87 and 94; 87 and 95; 87 and 96; 87 and 97; 87 and 98; 87 and 99; 87 and 100; 87 and 101; 87 and 102; 88 and 89; 88 and 90; 88 and 91; 88 and 92; 88 and 93; 88 and 94; 88 and 95; 88 and 96; 88 and 97; 88 and 98; 88 and 99; 88 and 100; 88 and 101; 88 and 102; 89 and 90; 89 and 91; 89 and 92; 89 and 93; 89 and 94; 89 and 95; 89 and 96; 89 and 97; 89 and 98; 89 and 99; 89 and 100; 89 and 101; 89 and 102; 90 and 91; 90 and 92; 90 and 93; 90 and 94; 90 and 95; 90 and 96; 90 and 97; 90 and 98; 90 and 99; 90 and 100; 90 and 101; 90 and 102; 91 and 92; 91 and 93; 91 and 94; 91 and 95; 91 and 96; 91 and 97; 91 and 98; 91 and 99; 91 and 100; 91 and 101; 91 and 102; 92 and 93; 92 and 94; 92 and 95; 92 and 96; 92 and 97; 92 and 98; 92 and 99; 92 and 100; 92 and 101; 92 and 102; 93 and 94; 93 and 95; 93 and 96; 93 and 97; 93 and 98; 93 and 99; 93 and 100; 93 and 101; 93 and 102; 94 and 95; 94 and 96; 94 and 97; 94 and 98; 94 and 99; 94 and 100; 94 and 101; 94 and 102; 95 and 96; 95 and 97; 95 and 98; 95 and 99; 95 and 100; 95 and 101; 95 and 102; 96 and 97; 96 and 98; 96 and 99; 96 and 100; 96 and 101; 96 and 102; 97 and 98; 97 and 99; 97 and 100; 97 and 101; 97 and 102; 98 and 99; 98 and 100; 98 and 101; 98 and 102; 99 and 100; 99 and 101; 99 and 102; 100 and 101; 100 and 102; or 101 and 102; and a second nucleic acid encoding a SaCas9.
Embodiment 11 is a composition comprising a first and a second nucleic acid molecule, wherein the first nucleic acid molecule encodes a Staphylococcus aureus Cas9 (SaCas9) endonuclease and optionally a first or a first and second guide RNA, and the second nucleic acid molecule comprises a first or a first and second guide RNA, wherein the first guide RNA comprises a first sequence and the second guide RNA comprises a second sequence from any one the following pairs of first and second sequences: SEQ ID NOs: 1 and 3; 16 and 17; 16 and 18; 16 and 19; 16 and 20; 16 and 21; 16 and 22; 16 and 23; 16 and 24; 16 and 25; 16 and 26; 17 and 18; 17 and 19; 17 and 20; 17 and 21; 17 and 22; 17 and 23; 17 and 24; 17 and 25; 17 and 26; 18 and 19; 18 and 20; 18 and 21; 18 and 22; 18 and 23; 18 and 24; 18 and 25; 18 and 26; 19 and 20; 19 and 21; 19 and 22; 19 and 23; 19 and 24; 19 and 25; 19 and 26; 20 and 21; 20 and 22; 20 and 23; 20 and 24; 20 and 25; 20 and 26; 21 and 22; 21 and 23; 21 and 24; 21 and 25; 21 and 26; 22 and 23; 22 and 24; 22 and 25; 22 and 26; 23 and 24; 23 and 25; 23 and 26; 24 and 25; 24 and 26; 25 and 26; 110 and 120; 110 and 121; 110 and 122; 110 and 123; 110 and 124; 110 and 125; 111 and 112; 111 and 113; 111 and 114; 111 and 115; 111 and 116; 111 and 117; 111 and 118; 111 and 119; 111 and 120; 111 and 121; 111 and 122; 111 and 123; 111 and 124; 111 and 125; 112 and 113; 112 and 114; 112 and 115; 112 and 116; 112 and 117; 112 and 118; 112 and 119; 112 and 120; 112 and 121; 112 and 122; 112 and 123; 112 and 124; 112 and 125; 113 and 114; 113 and 115; 113 and 116; 113 and 117; 113 and 118; 113 and 119; 113 and 120; 113 and 121; 113 and 122; 113 and 123; 113 and 124; 113 and 125; 114 and 115; 114 and 116; 114 and 117; 114 and 118; 114 and 119; 114 and 120; 114 and 121; 114 and 122; 114 and 123; 114 and 124; 114 and 125; 115 and 116; 115 and 117; 115 and 118; 115 and 119; 115 and 120; 115 and 121; 115 and 122; 115 and 123; 115 and 124; 115 and 125; 116 and 117; 116 and 118; 116 and 119; 116 and 120; 116 and 121; 116 and 122; 116 and 123; 116 and 124; 116 and 125; 117 and 118; 117 and 119; 117 and 120; 117 and 121; 117 and 122; 117 and 123; 117 and 124; 117 and 125; 118 and 119; 118 and 120; 118 and 121; 118 and 122; 118 and 123; 118 and 124; 118 and 125; 119 and 120; 119 and 121; 119 and 122; 119 and 123; 119 and 124; 119 and 125; 120 and 121; 120 and 122; 120 and 123; 120 and 124; 120 and 125; 121 and 122; 121 and 123; 121 and 124; 121 and 125; 122 and 123; 122 and 124; 122 and 125; 123 and 124; 123 and 125; 124 and 125; 148 and 149; 148 and 150; 148 and 151; 148 and 152; 148 and 153; 148 and 154; 148 and 155; 148 and 156; 148 and 157; 148 and 158; 148 and 159; 149 and 150; 149 and 151; 149 and 152; 149 and 153; 149 and 154; 149 and 155; 149 and 156; 149 and 157; 149 and 158; 149 and 159; 150 and 151; 150 and 152; 150 and 153; 150 and 154; 150 and 155; 150 and 156; 150 and 157; 150 and 158; 150 and 159; 151 and 152; 151 and 153; 151 and 154; 151 and 155; 151 and 156; 151 and 157; 151 and 158; 151 and 159; 152 and 153; 152 and 154; 152 and 155; 152 and 156; 152 and 157; 152 and 158; 152 and 159; 153 and 154; 153 and 155; 153 and 156; 153 and 157; 153 and 158; 153 and 159; 154 and 155; 154 and 156; 154 and 157; 154 and 158; 154 and 159; 155 and 156; 155 and 157; 155 and 158; 155 and 159; 156 and 157; 156 and 158; 156 and 159; 157 and 158; 157 and 159; 158 and 159; 71 and 72; 71 and 73; 71 and 74; 71 and 75; 71 and 76; 71 and 77; 71 and 78; 71 and 84; 71 and 85; 71 and 86; 71 and 87; 71 and 88; 71 and 89; 71 and 90; 71 and 91; 71 and 92; 71 and 93; 71 and 94; 71 and 95; 71 and 96; 71 and 97; 71 and 98; 71 and 99; 71 and 100; 71 and 101; 71 and 102; 72 and 73; 72 and 74; 72 and 75; 72 and 76; 72 and 77; 72 and 78; 72 and 84; 72 and 85; 72 and 86; 72 and 87; 72 and 88; 72 and 89; 72 and 90; 72 and 91; 72 and 92; 72 and 93; 72 and 94; 72 and 95; 72 and 96; 72 and 97; 72 and 98; 72 and 99; 72 and 100; 72 and 101; 72 and 102; 73 and 74; 73 and 75; 73 and 76; 73 and 77; 73 and 78; 73 and 84; 73 and 85; 73 and 86; 73 and 87; 73 and 88; 73 and 89; 73 and 90; 73 and 91; 73 and 92; 73 and 93; 73 and 94; 73 and 95; 73 and 96; 73 and 97; 73 and 98; 73 and 99; 73 and 100; 73 and 101; 73 and 102; 74 and 75; 74 and 76; 74 and 77; 74 and 78; 74 and 84; 74 and 85; 74 and 86; 74 and 87; 74 and 88; 74 and 89; 74 and 90; 74 and 91; 74 and 92; 74 and 93; 74 and 94; 74 and 95; 74 and 96; 74 and 97; 74 and 98; 74 and 99; 74 and 100; 74 and 101; 74 and 102; 75 and 76; 75 and 77; 75 and 78; 75 and 84; 75 and 85; 75 and 86; 75 and 87; 75 and 88; 75 and 89; 75 and 90; 75 and 91; 75 and 92; 75 and 93; 75 and 94; 75 and 95; 75 and 96; 75 and 97; 75 and 98; 75 and 99; 75 and 100; 75 and 101; 75 and 102; 76 and 77; 76 and 78; 76 and 84; 76 and 85; 76 and 86; 76 and 87; 76 and 88; 76 and 89; 76 and 90; 76 and 91; 76 and 92; 76 and 93; 76 and 94; 76 and 95; 76 and 96; 76 and 97; 76 and 98; 76 and 99; 76 and 100; 76 and 101; 76 and 102; 77 and 78; 77 and 84; 77 and 85; 77 and 86; 77 and 87; 77 and 88; 77 and 89; 77 and 90; 77 and 91; 77 and 92; 77 and 93; 77 and 94; 77 and 95; 77 and 96; 77 and 97; 77 and 98; 77 and 99; 77 and 100; 77 and 101; 77 and 102; 78 and 84; 78 and 85; 78 and 86; 78 and 87; 78 and 88; 78 and 89; 78 and 90; 78 and 91; 78 and 92; 78 and 93; 78 and 94; 78 and 95; 78 and 96; 78 and 97; 78 and 98; 78 and 99; 78 and 100; 78 and 101; 78 and 102; 84 and 85; 84 and 86; 84 and 87; 84 and 88; 84 and 89; 84 and 90; 84 and 91; 84 and 92; 84 and 93; 84 and 94; 84 and 95; 84 and 96; 84 and 97; 84 and 98; 84 and 99; 84 and 100; 84 and 101; 84 and 102; 85 and 86; 85 and 87; 85 and 88; 85 and 89; 85 and 90; 85 and 91; 85 and 92; 85 and 93; 85 and 94; 85 and 95; 85 and 96; 85 and 97; 85 and 98; 85 and 99; 85 and 100; 85 and 101; 85 and 102; 86 and 87; 86 and 88; 86 and 89; 86 and 90; 86 and 91; 86 and 92; 86 and 93; 86 and 94; 86 and 95; 86 and 96; 86 and 97; 86 and 98; 86 and 99; 86 and 100; 86 and 101; 86 and 102; 87 and 88; 87 and 89; 87 and 90; 87 and 91; 87 and 92; 87 and 93; 87 and 94; 87 and 95; 87 and 96; 87 and 97; 87 and 98; 87 and 99; 87 and 100; 87 and 101; 87 and 102; 88 and 89; 88 and 90; 88 and 91; 88 and 92; 88 and 93; 88 and 94; 88 and 95; 88 and 96; 88 and 97; 88 and 98; 88 and 99; 88 and 100; 88 and 101; 88 and 102; 89 and 90; 89 and 91; 89 and 92; 89 and 93; 89 and 94; 89 and 95; 89 and 96; 89 and 97; 89 and 98; 89 and 99; 89 and 100; 89 and 101; 89 and 102; 90 and 91; 90 and 92; 90 and 93; 90 and 94; 90 and 95; 90 and 96; 90 and 97; 90 and 98; 90 and 99; 90 and 100; 90 and 101; 90 and 102; 91 and 92; 91 and 93; 91 and 94; 91 and 95; 91 and 96; 91 and 97; 91 and 98; 91 and 99; 91 and 100; 91 and 101; 91 and 102; 92 and 93; 92 and 94; 92 and 95; 92 and 96; 92 and 97; 92 and 98; 92 and 99; 92 and 100; 92 and 101; 92 and 102; 93 and 94; 93 and 95; 93 and 96; 93 and 97; 93 and 98; 93 and 99; 93 and 100; 93 and 101; 93 and 102; 94 and 95; 94 and 96; 94 and 97; 94 and 98; 94 and 99; 94 and 100; 94 and 101; 94 and 102; 95 and 96; 95 and 97; 95 and 98; 95 and 99; 95 and 100; 95 and 101; 95 and 102; 96 and 97; 96 and 98; 96 and 99; 96 and 100; 96 and 101; 96 and 102; 97 and 98; 97 and 99; 97 and 100; 97 and 101; 97 and 102; 98 and 99; 98 and 100; 98 and 101; 98 and 102; 99 and 100; 99 and 101; 99 and 102; 100 and 101; 100 and 102; or 101 and 102.
Embodiment 12 is a composition comprising a first and a second nucleic acid molecule, wherein the first nucleic acid molecule encodes a Staphylococcus lugdunensis (SluCas9) endonuclease and optionally a first or a first and second guide RNA, and the second nucleic acid molecule comprises a first or a first and second guide RNA, wherein the first guide RNA comprises a first sequence and the second guide RNAs comprises a second sequence from any one the following pairs of first and second sequences: SEQ ID NOs: 200 and 201; 200 and 202; 200 and 203; 200 and 204; 200 and 205; 200 and 206; 200 and 207; 200 and 208; 201 and 202; 201 and 203; 201 and 204; 201 and 205; 201 and 206; 201 and 207; 201 and 208; 202 and 203; 202 and 204; 202 and 205; 202 and 206; 202 and 207; 202 and 208; 203 and 204; 203 and 205; 203 and 206; 203 and 207; 203 and 208; 204 and 205; 204 and 206; 204 and 207; 204 and 208; 205 and 206; 205 and 207; 205 and 208; 206 and 207; 206 and 208; 207 and 208; 231 and 232; 231 and 233; 231 and 234; 231 and 235; 231 and 236; 231 and 237; 231 and 238; 231 and 239; 231 and 240; 231 and 241; 231 and 242; 232 and 233; 232 and 234; 232 and 235; 232 and 236; 232 and 237; 232 and 238; 232 and 239; 232 and 240; 232 and 241; 232 and 242; 233 and 234; 233 and 235; 233 and 236; 233 and 237; 233 and 238; 233 and 239; 233 and 240; 233 and 241; 233 and 242; 234 and 235; 234 and 236; 234 and 237; 234 and 238; 234 and 239; 234 and 240; 234 and 241; 234 and 242; 235 and 236; 235 and 237; 235 and 238; 235 and 239; 235 and 240; 235 and 241; 235 and 242; 236 and 237; 236 and 238; 236 and 239; 236 and 240; 236 and 241; 236 and 242; 237 and 238; 237 and 239; 237 and 240; 237 and 241; 237 and 242; 238 and 239; 238 and 240; 238 and 241; 238 and 242; 239 and 240; 239 and 241; 239 and 242; 240 and 241; 240 and 242; 241 and 242; 270 and 271; 270 and 272; 270 and 273; 270 and 274; 270 and 275; 270 and 276; 270 and 277; 270 and 278; 270 and 279; 270 and 280; 270 and 281; 270 and 282; 270 and 283; 270 and 284; 270 and 285; 270 and 286; 270 and 287; 270 and 288; 270 and 289; 270 and 290; 270 and 291; 270 and 292; 271 and 272; 271 and 273; 271 and 274; 271 and 275; 271 and 276; 271 and 277; 271 and 278; 271 and 279; 271 and 280; 271 and 281; 271 and 282; 271 and 283; 271 and 284; 271 and 285; 271 and 286; 271 and 287; 271 and 288; 271 and 289; 271 and 290; 271 and 291; 271 and 292; 272 and 273; 272 and 274; 272 and 275; 272 and 276; 272 and 277; 272 and 278; 272 and 279; 272 and 280; 272 and 281; 272 and 282; 272 and 283; 272 and 284; 272 and 285; 272 and 286; 272 and 287; 272 and 288; 272 and 289; 272 and 290; 272 and 291; 272 and 292; 273 and 274; 273 and 275; 273 and 276; 273 and 277; 273 and 278; 273 and 279; 273 and 280; 273 and 281; 273 and 282; 273 and 283; 273 and 284; 273 and 285; 273 and 286; 273 and 287; 273 and 288; 273 and 289; 273 and 290; 273 and 291; 273 and 292; 274 and 275; 274 and 276; 274 and 277; 274 and 278; 274 and 279; 274 and 280; 274 and 281; 274 and 282; 274 and 283; 274 and 284; 274 and 285; 274 and 286; 274 and 287; 274 and 288; 274 and 289; 274 and 290; 274 and 291; 274 and 292; 275 and 276; 275 and 277; 275 and 278; 275 and 279; 275 and 280; 275 and 281; 275 and 282; 275 and 283; 275 and 284; 275 and 285; 275 and 286; 275 and 287; 275 and 288; 275 and 289; 275 and 290; 275 and 291; 275 and 292; 276 and 277; 276 and 278; 276 and 279; 276 and 280; 276 and 281; 276 and 282; 276 and 283; 276 and 284; 276 and 285; 276 and 286; 276 and 287; 276 and 288; 276 and 289; 276 and 290; 276 and 291; 276 and 292; 277 and 278; 277 and 279; 277 and 280; 277 and 281; 277 and 282; 277 and 283; 277 and 284; 277 and 285; 277 and 286; 277 and 287; 277 and 288; 277 and 289; 277 and 290; 277 and 291; 277 and 292; 278 and 279; 278 and 280; 278 and 281; 278 and 282; 278 and 283; 278 and 284; 278 and 285; 278 and 286; 278 and 287; 278 and 288; 278 and 289; 278 and 290; 278 and 291; 278 and 292; 279 and 280; 279 and 281; 279 and 282; 279 and 283; 279 and 284; 279 and 285; 279 and 286; 279 and 287; 279 and 288; 279 and 289; 279 and 290; 279 and 291; 279 and 292; 280 and 281; 280 and 282; 280 and 283; 280 and 284; 280 and 285; 280 and 286; 280 and 287; 280 and 288; 280 and 289; 280 and 290; 280 and 291; 280 and 292; 281 and 282; 281 and 283; 281 and 284; 281 and 285; 281 and 286; 281 and 287; 281 and 288; 281 and 289; 281 and 290; 281 and 291; 281 and 292; 282 and 283; 282 and 284; 282 and 285; 282 and 286; 282 and 287; 282 and 288; 282 and 289; 282 and 290; 282 and 291; 282 and 292; 283 and 284; 283 and 285; 283 and 286; 283 and 287; 283 and 288; 283 and 289; 283 and 290; 283 and 291; 283 and 292; 284 and 285; 284 and 286; 284 and 287; 284 and 288; 284 and 289; 284 and 290; 284 and 291; 284 and 292; 285 and 286; 285 and 287; 285 and 288; 285 and 289; 285 and 290; 285 and 291; 285 and 292; 286 and 287; 286 and 288; 286 and 289; 286 and 290; 286 and 291; 286 and 292; 287 and 288; 287 and 289; 287 and 290; 287 and 291; 287 and 292; 288 and 289; 288 and 290; 288 and 291; 288 and 292; 289 and 290; 289 and 291; 289 and 292; 290 and 291; 290 and 292; 291 and 292; 924 and 270; 924 and 272; 924 and 273; 924 and 274; 924 and 275; 924 and 276; 924 and 277; 924 and 278; 924 and 279; 924 and 280; 924 and 281; 924 and 282; 924 and 283; 924 and 284; 924 and 285; 924 and 286; 924 and 287; 924 and 288; 924 and 289; 924 and 290; 924 and 291; 924 and 292; 925 and 270; 925 and 272; 925 and 273; 925 and 274; 925 and 275; 925 and 276; 925 and 277; 925 and 278; 925 and 279; 925 and 280; 925 and 281; 925 and 282; 925 and 283; 925 and 284; 925 and 285; 925 and 286; 925 and 287; 925 and 288; 925 and 289; 925 and 290; 925 and 291; 925 and 292; 926 and 270; 926 and 272; 926 and 273; 926 and 274; 926 and 275; 926 and 276; 926 and 277; 926 and 278; 926 and 279; 926 and 280; 926 and 281; 926 and 282; 926 and 283; 926 and 284; 926 and 285; 926 and 286; 926 and 287; 926 and 288; 926 and 289; 926 and 290; 926 and 291; 926 and 292; 927 and 270; 927 and 272; 927 and 273; 927 and 274; 927 and 275; 927 and 276; 927 and 277; 927 and 278; 927 and 279; 927 and 280; 927 and 281; 927 and 282; 927 and 283; 927 and 284; 927 and 285; 927 and 286; 927 and 287; 927 and 288; 927 and 289; 927 and 290; 927 and 291; 927 and 292; 928 and 270; 928 and 272; 928 and 273; 928 and 274; 928 and 275; 928 and 276; 928 and 277; 928 and 278; 928 and 279; 928 and 280; 928 and 281; 928 and 282; 928 and 283; 928 and 284; 928 and 285; 928 and 286; 928 and 287; 928 and 288; 928 and 289; 928 and 290; 928 and 291; 928 and 292; 929 and 270; 929 and 272; 929 and 273; 929 and 274; 929 and 275; 929 and 276; 929 and 277; 929 and 278; 929 and 279; 929 and 280; 929 and 281; 929 and 282; 929 and 283; 929 and 284; 929 and 285; 929 and 286; 929 and 287; 929 and 288; 929 and 289; 929 and 290; 929 and 291; 929 and 292; 930 and 270; 930 and 272; 930 and 273; 930 and 274; 930 and 275; 930 and 276; 930 and 277; 930 and 278; 930 and 279; 930 and 280; 930 and 281; 930 and 282; 930 and 283; 930 and 284; 930 and 285; 930 and 286; 930 and 287; 930 and 288; 930 and 289; 930 and 290; 930 and 291; 930 and 292; 931 and 270; 931 and 272; 931 and 273; 931 and 274; 931 and 275; 931 and 276; 931 and 277; 931 and 278; 931 and 279; 931 and 280; 931 and 281; 931 and 282; 931 and 283; 931 and 284; 931 and 285; 931 and 286; 931 and 287; 931 and 288; 931 and 289; 931 and 290; 931 and 291; 931 and 292; 932 and 270; 932 and 272; 932 and 273; 932 and 274; 932 and 275; 932 and 276; 932 and 277; 932 and 278; 932 and 279; 932 and 280; 932 and 281; 932 and 282; 932 and 283; 932 and 284; 932 and 285; 932 and 286; 932 and 287; 932 and 288; 932 and 289; 932 and 290; 932 and 291; 932 and 292; 933 and 270; 933 and 272; 933 and 273; 933 and 274; 933 and 275; 933 and 276; 933 and 277; 933 and 278; 933 and 279; 933 and 280; 933 and 281; 933 and 282; 933 and 283; 933 and 284; 933 and 285; 933 and 286; 933 and 287; 933 and 288; 933 and 289; 933 and 290; 933 and 291; 933 and 292; 934 and 270; 934 and 272; 934 and 273; 934 and 274; 934 and 275; 934 and 276; 934 and 277; 934 and 278; 934 and 279; 934 and 280; 934 and 281; 934 and 282; 934 and 283; 934 and 284; 934 and 285; 934 and 286; 934 and 287; 934 and 288; 934 and 289; 934 and 290; 934 and 291; 934 and 292; 935 and 270; 935 and 272; 935 and 273; 935 and 274; 935 and 275; 935 and 276; 935 and 277; 935 and 278; 935 and 279; 935 and 280; 935 and 281; 935 and 282; 935 and 283; 935 and 284; 935 and 285; 935 and 286; 935 and 287; 935 and 288; 935 and 289; 935 and 290; 935 and 291; 935 and 292; 936 and 270; 936 and 272; 936 and 273; 936 and 274; 936 and 275; 936 and 276; 936 and 277; 936 and 278; 936 and 279; 936 and 280; 936 and 281; 936 and 282; 936 and 283; 936 and 284; 936 and 285; 936 and 286; 936 and 287; 936 and 288; 936 and 289; 936 and 290; 936 and 291; 936 and 292; 937 and 270; 937 and 272; 937 and 273; 937 and 274; 937 and 275; 937 and 276; 937 and 277; 937 and 278; 937 and 279; 937 and 280; 937 and 281; 937 and 282; 937 and 283; 937 and 284; 937 and 285; 937 and 286; 937 and 287; 937 and 288; 937 and 289; 937 and 290; 937 and 291; 937 and 292; 938 and 270; 938 and 272; 938 and 273; 938 and 274; 938 and 275; 938 and 276; 938 and 277; 938 and 278; 938 and 279; 938 and 280; 938 and 281; 938 and 282; 938 and 283; 938 and 284; 938 and 285; 938 and 286; 938 and 287; 938 and 288; 938 and 289; 938 and 290; 938 and 291; 938 and 292; 950 and 275; 951 and 275; 952 and 275; 953 and 275; 954 and 275; or 955 and 275.
Embodiment 13 is a composition comprising a first and a second nucleic acid molecule, wherein the first nucleic acid molecule encodes a sRGN endonuclease (e.g., sRGN3.1, sRGN3.3, or sRGN4) and optionally a first or a first and second guide RNA, and the second nucleic acid molecule comprises a first or a first and second guide RNA, wherein the first guide RNA comprises a first sequence and the second guide RNAs comprises a second sequence from any one the following pairs of first and second sequences: SEQ ID NOs: 200 and 201; 200 and 202; 200 and 203; 200 and 204; 200 and 205; 200 and 206; 200 and 207; 200 and 208; 201 and 202; 201 and 203; 201 and 204; 201 and 205; 201 and 206; 201 and 207; 201 and 208; 202 and 203; 202 and 204; 202 and 205; 202 and 206; 202 and 207; 202 and 208; 203 and 204; 203 and 205; 203 and 206; 203 and 207; 203 and 208; 204 and 205; 204 and 206; 204 and 207; 204 and 208; 205 and 206; 205 and 207; 205 and 208; 206 and 207; 206 and 208; 207 and 208; 231 and 232; 231 and 233; 231 and 234; 231 and 235; 231 and 236; 231 and 237; 231 and 238; 231 and 239; 231 and 240; 231 and 241; 231 and 242; 232 and 233; 232 and 234; 232 and 235; 232 and 236; 232 and 237; 232 and 238; 232 and 239; 232 and 240; 232 and 241; 232 and 242; 233 and 234; 233 and 235; 233 and 236; 233 and 237; 233 and 238; 233 and 239; 233 and 240; 233 and 241; 233 and 242; 234 and 235; 234 and 236; 234 and 237; 234 and 238; 234 and 239; 234 and 240; 234 and 241; 234 and 242; 235 and 236; 235 and 237; 235 and 238; 235 and 239; 235 and 240; 235 and 241; 235 and 242; 236 and 237; 236 and 238; 236 and 239; 236 and 240; 236 and 241; 236 and 242; 237 and 238; 237 and 239; 237 and 240; 237 and 241; 237 and 242; 238 and 239; 238 and 240; 238 and 241; 238 and 242; 239 and 240; 239 and 241; 239 and 242; 240 and 241; 240 and 242; 241 and 242; 270 and 271; 270 and 272; 270 and 273; 270 and 274; 270 and 275; 270 and 276; 270 and 277; 270 and 278; 270 and 279; 270 and 280; 270 and 281; 270 and 282; 270 and 283; 270 and 284; 270 and 285; 270 and 286; 270 and 287; 270 and 288; 270 and 289; 270 and 290; 270 and 291; 270 and 292; 271 and 272; 271 and 273; 271 and 274; 271 and 275; 271 and 276; 271 and 277; 271 and 278; 271 and 279; 271 and 280; 271 and 281; 271 and 282; 271 and 283; 271 and 284; 271 and 285; 271 and 286; 271 and 287; 271 and 288; 271 and 289; 271 and 290; 271 and 291; 271 and 292; 272 and 273; 272 and 274; 272 and 275; 272 and 276; 272 and 277; 272 and 278; 272 and 279; 272 and 280; 272 and 281; 272 and 282; 272 and 283; 272 and 284; 272 and 285; 272 and 286; 272 and 287; 272 and 288; 272 and 289; 272 and 290; 272 and 291; 272 and 292; 273 and 274; 273 and 275; 273 and 276; 273 and 277; 273 and 278; 273 and 279; 273 and 280; 273 and 281; 273 and 282; 273 and 283; 273 and 284; 273 and 285; 273 and 286; 273 and 287; 273 and 288; 273 and 289; 273 and 290; 273 and 291; 273 and 292; 274 and 275; 274 and 276; 274 and 277; 274 and 278; 274 and 279; 274 and 280; 274 and 281; 274 and 282; 274 and 283; 274 and 284; 274 and 285; 274 and 286; 274 and 287; 274 and 288; 274 and 289; 274 and 290; 274 and 291; 274 and 292; 275 and 276; 275 and 277; 275 and 278; 275 and 279; 275 and 280; 275 and 281; 275 and 282; 275 and 283; 275 and 284; 275 and 285; 275 and 286; 275 and 287; 275 and 288; 275 and 289; 275 and 290; 275 and 291; 275 and 292; 276 and 277; 276 and 278; 276 and 279; 276 and 280; 276 and 281; 276 and 282; 276 and 283; 276 and 284; 276 and 285; 276 and 286; 276 and 287; 276 and 288; 276 and 289; 276 and 290; 276 and 291; 276 and 292; 277 and 278; 277 and 279; 277 and 280; 277 and 281; 277 and 282; 277 and 283; 277 and 284; 277 and 285; 277 and 286; 277 and 287; 277 and 288; 277 and 289; 277 and 290; 277 and 291; 277 and 292; 278 and 279; 278 and 280; 278 and 281; 278 and 282; 278 and 283; 278 and 284; 278 and 285; 278 and 286; 278 and 287; 278 and 288; 278 and 289; 278 and 290; 278 and 291; 278 and 292; 279 and 280; 279 and 281; 279 and 282; 279 and 283; 279 and 284; 279 and 285; 279 and 286; 279 and 287; 279 and 288; 279 and 289; 279 and 290; 279 and 291; 279 and 292; 280 and 281; 280 and 282; 280 and 283; 280 and 284; 280 and 285; 280 and 286; 280 and 287; 280 and 288; 280 and 289; 280 and 290; 280 and 291; 280 and 292; 281 and 282; 281 and 283; 281 and 284; 281 and 285; 281 and 286; 281 and 287; 281 and 288; 281 and 289; 281 and 290; 281 and 291; 281 and 292; 282 and 283; 282 and 284; 282 and 285; 282 and 286; 282 and 287; 282 and 288; 282 and 289; 282 and 290; 282 and 291; 282 and 292; 283 and 284; 283 and 285; 283 and 286; 283 and 287; 283 and 288; 283 and 289; 283 and 290; 283 and 291; 283 and 292; 284 and 285; 284 and 286; 284 and 287; 284 and 288; 284 and 289; 284 and 290; 284 and 291; 284 and 292; 285 and 286; 285 and 287; 285 and 288; 285 and 289; 285 and 290; 285 and 291; 285 and 292; 286 and 287; 286 and 288; 286 and 289; 286 and 290; 286 and 291; 286 and 292; 287 and 288; 287 and 289; 287 and 290; 287 and 291; 287 and 292; 288 and 289; 288 and 290; 288 and 291; 288 and 292; 289 and 290; 289 and 291; 289 and 292; 290 and 291; 290 and 292; 291 and 292; 924 and 270; 924 and 272; 924 and 273; 924 and 274; 924 and 275; 924 and 276; 924 and 277; 924 and 278; 924 and 279; 924 and 280; 924 and 281; 924 and 282; 924 and 283; 924 and 284; 924 and 285; 924 and 286; 924 and 287; 924 and 288; 924 and 289; 924 and 290; 924 and 291; 924 and 292; 925 and 270; 925 and 272; 925 and 273; 925 and 274; 925 and 275; 925 and 276; 925 and 277; 925 and 278; 925 and 279; 925 and 280; 925 and 281; 925 and 282; 925 and 283; 925 and 284; 925 and 285; 925 and 286; 925 and 287; 925 and 288; 925 and 289; 925 and 290; 925 and 291; 925 and 292; 926 and 270; 926 and 272; 926 and 273; 926 and 274; 926 and 275; 926 and 276; 926 and 277; 926 and 278; 926 and 279; 926 and 280; 926 and 281; 926 and 282; 926 and 283; 926 and 284; 926 and 285; 926 and 286; 926 and 287; 926 and 288; 926 and 289; 926 and 290; 926 and 291; 926 and 292; 927 and 270; 927 and 272; 927 and 273; 927 and 274; 927 and 275; 927 and 276; 927 and 277; 927 and 278; 927 and 279; 927 and 280; 927 and 281; 927 and 282; 927 and 283; 927 and 284; 927 and 285; 927 and 286; 927 and 287; 927 and 288; 927 and 289; 927 and 290; 927 and 291; 927 and 292; 928 and 270; 928 and 272; 928 and 273; 928 and 274; 928 and 275; 928 and 276; 928 and 277; 928 and 278; 928 and 279; 928 and 280; 928 and 281; 928 and 282; 928 and 283; 928 and 284; 928 and 285; 928 and 286; 928 and 287; 928 and 288; 928 and 289; 928 and 290; 928 and 291; 928 and 292; 929 and 270; 929 and 272; 929 and 273; 929 and 274; 929 and 275; 929 and 276; 929 and 277; 929 and 278; 929 and 279; 929 and 280; 929 and 281; 929 and 282; 929 and 283; 929 and 284; 929 and 285; 929 and 286; 929 and 287; 929 and 288; 929 and 289; 929 and 290; 929 and 291; 929 and 292; 930 and 270; 930 and 272; 930 and 273; 930 and 274; 930 and 275; 930 and 276; 930 and 277; 930 and 278; 930 and 279; 930 and 280; 930 and 281; 930 and 282; 930 and 283; 930 and 284; 930 and 285; 930 and 286; 930 and 287; 930 and 288; 930 and 289; 930 and 290; 930 and 291; 930 and 292; 931 and 270; 931 and 272; 931 and 273; 931 and 274; 931 and 275; 931 and 276; 931 and 277; 931 and 278; 931 and 279; 931 and 280; 931 and 281; 931 and 282; 931 and 283; 931 and 284; 931 and 285; 931 and 286; 931 and 287; 931 and 288; 931 and 289; 931 and 290; 931 and 291; 931 and 292; 932 and 270; 932 and 272; 932 and 273; 932 and 274; 932 and 275; 932 and 276; 932 and 277; 932 and 278; 932 and 279; 932 and 280; 932 and 281; 932 and 282; 932 and 283; 932 and 284; 932 and 285; 932 and 286; 932 and 287; 932 and 288; 932 and 289; 932 and 290; 932 and 291; 932 and 292; 933 and 270; 933 and 272; 933 and 273; 933 and 274; 933 and 275; 933 and 276; 933 and 277; 933 and 278; 933 and 279; 933 and 280; 933 and 281; 933 and 282; 933 and 283; 933 and 284; 933 and 285; 933 and 286; 933 and 287; 933 and 288; 933 and 289; 933 and 290; 933 and 291; 933 and 292; 934 and 270; 934 and 272; 934 and 273; 934 and 274; 934 and 275; 934 and 276; 934 and 277; 934 and 278; 934 and 279; 934 and 280; 934 and 281; 934 and 282; 934 and 283; 934 and 284; 934 and 285; 934 and 286; 934 and 287; 934 and 288; 934 and 289; 934 and 290; 934 and 291; 934 and 292; 935 and 270; 935 and 272; 935 and 273; 935 and 274; 935 and 275; 935 and 276; 935 and 277; 935 and 278; 935 and 279; 935 and 280; 935 and 281; 935 and 282; 935 and 283; 935 and 284; 935 and 285; 935 and 286; 935 and 287; 935 and 288; 935 and 289; 935 and 290; 935 and 291; 935 and 292; 936 and 270; 936 and 272; 936 and 273; 936 and 274; 936 and 275; 936 and 276; 936 and 277; 936 and 278; 936 and 279; 936 and 280; 936 and 281; 936 and 282; 936 and 283; 936 and 284; 936 and 285; 936 and 286; 936 and 287; 936 and 288; 936 and 289; 936 and 290; 936 and 291; 936 and 292; 937 and 270; 937 and 272; 937 and 273; 937 and 274; 937 and 275; 937 and 276; 937 and 277; 937 and 278; 937 and 279; 937 and 280; 937 and 281; 937 and 282; 937 and 283; 937 and 284; 937 and 285; 937 and 286; 937 and 287; 937 and 288; 937 and 289; 937 and 290; 937 and 291; 937 and 292; 938 and 270; 938 and 272; 938 and 273; 938 and 274; 938 and 275; 938 and 276; 938 and 277; 938 and 278; 938 and 279; 938 and 280; 938 and 281; 938 and 282; 938 and 283; 938 and 284; 938 and 285; 938 and 286; 938 and 287; 938 and 288; 938 and 289; 938 and 290; 938 and 291; 938 and 292; 950 and 275; 951 and 275; 952 and 275; 953 and 275; 954 and 275; or 955 and 275.
Embodiment 14 is a composition of embodiment 13, wherein the sRGN endonuclease comprises an amino acid sequence that is at least 80%, 85%, 90%, 91%, 92%, 93%, 94%, 95%, 96%, 97%, 98%, 99%, or 100% identical to the sequence of any one of SEQ ID Nos: 7024, 7026, or 7027.
Embodiment 15 is a composition of embodiment 13 or 14, wherein the sRGN endonuclease is encoded by a nucleic acid sequence that is at least 80%, 85%, 90%, 91%, 92%, 93%, 94%, 95%, 96%, 97%, 98%, 99%, or 100% identical to the sequence of any one of SEQ ID Nos: 917, 919 or 920.
Embodiment 16 is a composition of any one of the preceding embodiments, comprising at least two guide RNAs, wherein once expressed in vitro or in vivo in combination with an RNA-guided endonuclease, the at least two guide RNAs facilitate a 3n+1 edit of exon 45, 51 or 53 of the dystrophin gene, wherein “n” is any negative whole number (e.g., any whole number between −10 and −75).
Embodiment 17 is a composition of any one of the preceding embodiments, comprising at least two guide RNAs, wherein once expressed in vitro or in vivo in combination with an RNA-guided endonuclease, the at least two guide RNAs target exon 45, 51 or 53 of the dystrophin gene and are capable of excising a nucleic acid that is at least 26, 29, 32, 35, 38, 41, 44, 47, 50, 53, 56, 59, 62, 65, 68, 71, 74, 77, 80, 83, 86, 89, 92, 95, 98, 101, 104, 107, 110, 113, 116, 119, 122, 125, 128, 131, 134, 137, 140, 143, 146, 149, 152, 155, 158, 161, 164, 167, 170, 173, 176, 179, 182, 185, 188, 191, 194, 197, 200, 203, 206, 209, 212, 215, or 218 nucleotides in length.
Embodiment 18 is a composition of any one of the preceding embodiments, comprising at least two guide RNAs, wherein once expressed in vitro or in vivo in combination with an RNA-guided endonuclease, the at least two guide RNAs facilitate a 3n+2 edit of exon 44 or 50 of the dystrophin gene, wherein “n” is any negative whole number (e.g., any whole number between −10 and −75).
Embodiment 19 is a composition of any one of the preceding embodiments, comprising at least two guide RNAs, wherein once expressed in vitro or in vivo in combination with an RNA-guided endonuclease, the at least two guide RNAs target exon 44 or 50 of the dystrophin gene and are capable of excising a nucleic acid that is at least 25, 28, 31, 34, 37, 40, 43, 46, 49, 52, 55, 58, 61, 64, 67, 70, 73, 76, 79, 82, 85, 88, 91, 94, 97, 100, 103, 106, 109, 112, 115, 118, 121, 124, 127, 130, 133, 136, 139, 142, 145, 148, 151, 154, 157, 160, 163, 166, 169, 172, 175, 178, 181, 184, 187, 190, 193, 196, 199, 202, 205, 208, 211, 214, or 217 nucleotides in length.
Embodiment 20 is a composition of any one of the preceding embodiments, comprising at least two guide RNAs, wherein once expressed in vitro or in vivo in combination with an RNA-guided endonuclease, the at least two guide RNAs excise a portion of an exon, wherein the size of the excised portion of the exon is between 5 and 250 nucleotides in length.
Embodiment 21 is a composition of any one of the preceding embodiments, comprising at least two guide RNAs, wherein once expressed in vitro or in vivo in combination with an RNA-guided endonuclease, the at least two guide RNAs excise a portion of an exon, wherein the size of the excised portion of the exon is between 5 and 250, 5 and 200, 5 and 150, 5 and 100, 5 and 75, 5 and 50, 5 and 25, 5 and 10, 20 and 250, 20 and 200, 20 and 150, 20 and 100, 20 and 75, 20 and 50, 20 and 25, 50 and 250, 50 and 200, 50 and 150, 50 and 100, and 50 and 75 nucleotides.
Embodiment 22 is a composition of any one of the preceding embodiments, comprising at least two guide RNAs, wherein once expressed in vitro or in vivo in combination with an RNA-guided endonuclease, the at least two guide RNAs excise a portion of the exon, wherein the size of the excised portion of the exon is between 8 and 167 nucleotides.
Embodiment 23 is a composition of any one of the preceding embodiments, wherein the one or more guide RNAs is an sgRNA.
Embodiment 24 is a composition of any one of the preceding embodiments, wherein the one or more guide RNAs is modified.
Embodiment 25 is a composition of any one of the preceding embodiments, wherein the one or more guide RNAs or nucleic acids are in a vector.
Embodiment 26 is a composition of embodiment 25, wherein the vector is a viral vector.
Embodiment 27 is a composition of embodiment 26, wherein the viral vector is an AAV vector.
Embodiment 28 is a composition of embodiment 27, wherein the AAV vector is an AAV9 vector.
Embodiment 29 is a composition of any one of the preceding embodiments, wherein the promoter for the one or more guide RNAs is hU6c.
Embodiment 30 is a composition of any one of the preceding embodiments, wherein the one or more guide RNAs is a guide RNA for SaCas9, and the one or more guide RNAs comprise a scaffold comprising the sequence of SEQ ID NO: 504.
Embodiment 31 is a composition of any one of the preceding embodiments, wherein the one or more guide RNAs is a guide RNA for SluCas9, and the one or more guide RNAs comprise a scaffold comprising the sequence of SEQ ID NO: 901.
Embodiment 32 is composition of any one of the preceding embodiments, wherein the one or more guide RNAs is in an AAV vector, wherein the vector comprises from 5′ to 3′ with respect to the plus strand: the reverse complement of a first guide RNA scaffold sequence; the reverse complement of a nucleic acid encoding a first guide RNA sequence; the reverse complement of a promoter for expression of the nucleic acid encoding the first guide RNA sequence; a promoter (e.g., CK8e) for expression of a nucleic acid encoding SaCas9, SluCas9, or a sRGN; a nucleic acid encoding a SaCas9, SluCas9, or a sRGN (e.g., sRGN3.1, sRGN3,3, or sRGN4); a polyadenylation sequence; a promoter for expression of a second guide RNA sequence in the same direction as the promoter for SaCas9, SluCas9, or the sRGN; a second guide RNA guide sequence; and a second guide RNA scaffold sequence.
Embodiment 33 is a composition of embodiment 32, wherein:
   i) the first guide RNA sequence comprises the sequence of SEQ ID NO: 271 or 281, and the second guide RNA sequence comprises the sequence of SEQ ID NO: 275; or
   ii) the first guide RNA sequence comprises the sequence of SEQ ID NO: 283, and the second guide RNA sequence comprises the sequence of SEQ ID NO: 290.
Embodiment 34 is a composition of embodiment 32 or 33, wherein the promoter for expression of a nucleic acid encoding SaCas9, SluCas9, or a sRGN is a promoter for expression of a sRGN; wherein the nucleic acid encoding a SaCas9, SluCas9, or a sRGN encodes for a sRGN, and wherein the sRGN comprises an amino acid sequence that is at least 80%, 85%, 90%, 91%, 92%, 93%, 94%, 95%, 96%, 97%, 98%, 99%, or 100% identical to the sequence of any one of SEQ ID Nos: 7024, 7026, or 7027.
Embodiment 35 is a composition of embodiment 34, wherein the sRGN is encoded by a nucleic acid sequence that is at least 80%, 85%, 90%, 91%, 92%, 93%, 94%, 95%, 96%, 97%, 98%, 99%, or 100% identical to the sequence of any one of SEQ ID Nos: 917, 919 or 920.
Embodiment 36 is a composition of any one of embodiments 1-35, wherein the one or more guide RNA sequences, or the first and/or second guide RNA sequence of the pair of guide RNAs, is no more than 16, no more than 17, no more than 18, no more than 19, no more than 20, no more than 21, or no more than 22 nucleotides in length.
Embodiment 37 is a method of treating Duchenne Muscular Dystrophy (DMD), the method comprising delivering to a cell the composition of any one of embodiments 1-36.
Embodiment 38 is a method of excising a portion of the DMD gene, the method comprising delivering to a cell the composition of any one of embodiments 1-36, wherein the size of the excised portion is less than about 250 nucleotides.

### DESCRIPTION OF FIGURES

FIG. 1 shows editing frequency and indel profiles of selected SaCas9, SaCas9-KKH and SluCas9 sgRNA pairs targeting Exon 51 of DMD gene in HEK293FT cells. Each stack bar represents a sgRNA pair and the bar height depicts the average frequency of the total edit. Different indel profiles for each pair of sgRNAs are represented by using distinct patterns as shown in the key. Error bar denotes the standard deviation for each indel group. The “Guide IDs” in the figure are shown as numbers only and generally correspond to the last digit in the “Guide RNA Name” column of Table 6 for each provided Cas9 type. “RF +1” means a reframing edit by insertion of a single nucleotide. “RF Other” means other reframing edits. “Exon Skipping” means 1) indels overlap with the splice acceptor (AG) (5′-end of the target exon); 2) deletion with ≥9-nt overlap with the splicing window at one of the two guides; or 3) insertion at the exact GT/AG splicing sites and with length≥9-nt at one of the two guides.

FIGS. 2A and 2B show editing frequency and indel profile of selected SaCas9 and SluCas9 sgRNA pairs targeting Exon 45 or Exon 51 of DMD gene in Human Skeletal Muscle Myoblasts (HsMM) cells. Each stack bar represents a sgRNA pair and the bar height depicts the average frequency of the total edit. Different indel profiles for each pair of sgRNAs are represented by using distinct patterns. FIG. 2A shows editing frequency and profile as calculated by Inference of CRISPR Edit (ICE) via Sanger sequencing signal decomposition. FIG. 2B shows editing frequency and profile as calculated by next generation sequencing (NGS). NA denotes data not available due to the failed QC at the step of analysis. Error bars denote the standard deviation for each indel group. The “Guide IDs” in the figure are shown as numbers only and generally correspond to the last digit in the “Guide RNA Name” column of Table 6 for each provided Cas9 type. “RF +1” means a refraining edit by insertion of a single nucleotide. “RF Other” means other refraining edits. “Exon Skipping” means 1) indels overlap with the splice acceptor (AG) (5′-end of the target exon); 2) deletion with ≥9-nt overlap with the splicing window at one of the two guides; or 3) insertion at the exact GT/AG splicing sites and with length≥9-nt at one of the two guides.

FIG. 3 shows editing frequency and indel profile of selected SaCas9, SaCas9-KKH and SluCas9 sgRNA pairs within exon 53 in HEK293FT cells. Each bar represents a sgRNA or sgRNA pair and the bar height depicts the average total indel frequency. Indel profiles are shown for each sgRNA using distinct patterns. Error bar=standard deviation for each indel group. The guide RNAs shown in the figure generally correspond to the “Guide RNA Name” column of Table 6 (e.g., E53SL4 in FIG. 3 corresponds to E53Slu4 in Table 6, E53SL23 in FIG. 3 corresponds to E53Slu23). “+1 bp” means a refraining edit by insertion of a single nucleotide.

FIGS. 4A-B show the method of determining exemplary guide pairs for exons 44 and 50. FIG. 4A shows the refraining for the respective exons (44 and 50) based on neighboring exons. FIG. 4B shows the refraining window and last premature stop codon for exon 44 or 50.

FIGS. 5A-B are schematics showing six representative vector designs. White arrows indicate directionality of expression of the sgRNA(s), while the black arrows indicate directionality of the Cas9 protein. In a particular embodiment, the Cas9 promoter may be CK8e. “Pol III” refers to a representative promoter for the expression of sgRNAs, “g1” and “g2” each refer to a guide sequence, “scaffold” refers to the scaffold of a guide RNA, and “pa” refers to a polyadenylation sequence. FIG. 5A shows Designs 1-4. FIG. 5B shows Designs 5 and 6, which are specific variations of Design 2 (shown generically in FIG. 5A), wherein the Cas9 is SluCas9, with specific guide sequences, promoters, scaffold, and NLS sequences. Design 5 and Design 6 differ only in the order in which the guide sequences are presented. Design 5 depicts the Slu7 guide sequence in the first guide position and the Slu3 guide in the second guide position. Design 6 depicts the Slu3 guide sequence in the first guide position and the Slu7 guide sequence in the second guide position.

FIGS. 6A and 6B show editing frequency and indel profile of selected SluCas9 sgRNA pairs targeting Exon 45 or Exon 51 of the DMD gene in HsMM cells. Each stack bar represents a sgRNA pair and the bar height depicts the average frequency of the total edit. Different indel profiles for each pair of sgRNAs are represented by using distinct patterns. FIG. 6A shows editing frequency and profile as calculated by next generation sequencing (NGS) for the guide pairs, where E51Slu31 is targeting the refraining window of Exon 51 of the DMD gene. FIG. 6B shows editing frequency and profile as calculated by next generation sequencing (NGS) for the guide pairs, where E51Slu10 is targeting the refraining window of Exon 51 of the DMD gene. Error bars denote the standard deviation for each indel group. The “Guide IDs” in the figure are shown as numbers only and generally correspond to the last digit in the “Guide RNA Name” column of Table 2 for each provided Cas9 type. “RF Other” means other refraining edits.

FIGS. 7A and 7B show editing frequency and indel profile of selected SluCas9 sgRNA pairs targeting various exons of the DMD gene in HsMM cells. FIG. 7A shows editing frequency and indel profile of selected SluCas9 sgRNA pairs targeting Exon 51 of the DMD gene in HsMM cells. FIG. 7B shows editing frequency and indel profile of selected SluCas9 sgRNA pairs targeting Exon 45 or Exon 51 of the DMD gene in HsMM cells. In FIGS. 7A and 7B, each stack bar represents a sgRNA pair and the bar height depicts the average frequency of the total edit. Different indel profiles for each pair of sgRNAs are represented by using distinct patterns. Editing frequencies and profiles are as calculated by next generation sequencing (NGS) for three guide pairs, in three doses of RNP; high (H), medium (M), and low (L). Error bars denote the standard deviation for each indel group. The “Guide IDs” in the figures are shown as numbers only and generally correspond to the last digit in the “Guide RNA Name” column of Table 2 for each provided Cas9 type. “RF Other” means other refraining edits.

FIGS. 8A and 8B show editing frequency and indel profile of selected SluCas9 sgRNA pairs targeting Exon 45 or Exon 51 of the DMD gene in HsMM cells. Each stack bar represents a sgRNA pair and the bar height depicts the average frequency of the total edit. Different indel profiles for each pair of sgRNAs are represented by using distinct patterns. FIG. 8A shows editing frequency and profile as calculated by next generation sequencing (NGS) for the E51Slu10 and E51Slu16 guide pair, where ribonucleoprotein (RNP) stoichiometry varies across the sample set. FIG. 8B shows editing frequency and profile as calculated by next generation sequencing (NGS) for the E51Slu10 and E51Slu26 guide pair, where ribonucleoprotein (RNP) stoichiometry varies across the sample set. Error bars denote the standard deviation for each indel group. The “Guide IDs” in the figure are shown as numbers only and generally correspond to the last digit in the “Guide RNA Name” column of Table 2 for each provided Cas9 type. “RF Other” means other reframing edits.

FIG. 9 shows the editing frequency of several nucleases (SluCas9, sRGNs 3.1, 3.3, 4) with various sgRNA pairs (Slu14/7, Slu16/23, Slu3/7) that target exon 53 of the DMD gene in human HEK293FT cells. One high-performing sgRNA for exon 45 of the DMD gene was included as a reference (E45Slu18/4). The Slu_v5 scaffold (SEQ ID NO: 901) was used for all samples, except for one triplicate of a E45Slu18/4 sample. Boxplot represents the average total indel frequency of each sgRNA pair. Data are the average of 2-3 replicates. In general, the “gRNAs” in the figure are shown as numbers only and generally correspond to the last digit in the “Guide RNA Name” column of Table 5C and 5D for each provided Cas9 type.

FIG. 10 shows the editing frequency and indel profile of several nucleases with various sgRNA pairs that target exon 53 of the DMD gene in human HEK293FT cells. One high-performing sgRNA for exon 45 of the DMD gene was included as a reference (E45Slu18/4). Each bar represents a sgRNA pair and the bar height depicts the average total indel frequency. Indel profiles are shown for each sgRNA as follows: black (precision deletion leading to refraining, sometimes referred to as “Precise Deletion”); vertical lines (indels other than precision deletion leading to reframing, sometimes referred to as “Other reframing”); white (all other indels, sometimes referred to as “Other indels”). Data are the average of 2-3 replicates. In general, the “gRNAs” in the figure are shown as numbers only and generally correspond to the last digit in the “Guide RNA Name” column of Table 5C and 5D for each provided Cas9 type.

FIG. 11 shows a bar graph representing the percentage of productive indels of several nucleases (SluCas9, sRGNs 3.1, 3.3, 4) with various sgRNA pairs that target exon 53 of the DMD gene in human HEK293FT cells. Y-axis represents the percentage of precision deletions over total indels. Data are the average of 2-3 replicates. In general, the “gRNAs” in the figure are shown as numbers only and generally correspond to the last digit in the “Guide RNA Name” column of Table 5C and 5D for each provided Cas9 type.

FIG. 12 shows a bar graph representing the editing frequency and indel profile of several nucleases (SluCas9, sRGNs 3.1, 3.3, 4) with two different sgRNA pairs (16+23, and 3+7) targeting exon 53 of the DMD gene in primary muscle skeletal myoblast (HsMM) cells. Each bar represents a sgRNA pair and the bar height depicts the average total indel frequency (%). Indel profiles are shown for each sgRNA as follows: black green (precision deletion leading to reframing, sometimes referred to as “Precise Deletion”); vertical lines (indels other than precision deletion leading to refraining, sometimes referred to as “Other reframing”); white (all other indels, sometimes referred to as “Other indels”). Data are the average of 3 replicates.

### DETAILED DESCRIPTION

Reference will now be made in detail to certain embodiments of the invention, examples of which are illustrated in the accompanying drawings. While the invention is described in conjunction with the illustrated embodiments, it will be understood that they are not intended to limit the invention to those embodiments. On the contrary, the invention is intended to cover all alternatives, modifications, and equivalents, which may be included within the invention as defined by the appended claims and included embodiments.

Before describing the present teachings in detail, it is to be understood that the disclosure is not limited to specific compositions or process steps, as such may vary. It should be noted that, as used in this specification and the appended claims, the singular form “a”, “an”, and “the” include plural references unless the context clearly dictates otherwise. Thus, for example, reference to “a guide” includes a plurality of guides and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### I. Definitions

Unless stated otherwise, the following terms and phrases as used herein are intended to have the following meanings:

“Polynucleotide,” “nucleic acid,” and “nucleic acid molecule,” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA.

“Guide RNA” and simply “guide” are used herein interchangeably to refer to either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” refers to each type. The trRNA may be a naturally-occurring sequence, or a trRNA sequence with modifications or variations compared to naturally-occurring sequences. For clarity, the terms “guide RNA” or “guide” as used herein, and unless specifically stated otherwise, may refer to an RNA molecule (comprising A, C, G, and U nucleotides) or to a DNA molecule encoding such an RNA molecule (comprising A, C, G, and T nucleotides) or complementary sequences thereof. In general, in the case of a DNA nucleic acid construct encoding a guide RNA, the U residues in any of the RNA sequences described herein may be replaced with T residues, and in the case of a guide RNA construct encoded by any of the DNA sequences described herein, the T residues may be replaced with U residues.

As used herein, a “spacer sequence,” sometimes also referred to herein and in the literature as a “spacer,” “protospacer,” “guide sequence,” or “targeting sequence” refers to a sequence within a guide RNA that is complementary to a target sequence and functions to direct a guide RNA to a target sequence for cleavage by a Cas9. For clarity, the terms “spacer sequence”, “spacer,” “protospacer,” “guide sequence,” or “targeting sequence” as used herein, and unless specifically stated otherwise, may refer to an RNA molecule (comprising A, C, G, and U nucleotides) or to a DNA molecule encoding such an RNA molecule (comprising A, C, G, and T nucleotides) or complementary sequences thereof. A guide sequence can be 24, 23, 22, 21, 20 or fewer base pairs in length, e.g., in the case of Staphylococcus lugdunensis (i.e., SluCas9) or Staphylococcus aureus (i.e., SaCas9) and related Cas9 homologs/orthologs. In preferred embodiments, a guide/spacer sequence in the case of SluCas9 or SaCas9 is at least 20 base pairs in length, or more specifically, within 20-25 base pairs in length (see, e.g., Schmidt et al., 2021, Nature Communications, “Improved CRISPR genome editing using small highly active and specific engineered RNA-guided nucleases”). Shorter or longer sequences can also be used as guides, e.g., 15-, 16-, 17-, 18-, 19-, 20-, 21-, 22-, 23-, 24-, or 25-nucleotides in length. For example, in some embodiments, the guide sequence comprises at least 16, 17, 18, 19, 20, 21, 22, 23, 24, or 25 contiguous nucleotides of a sequence selected from SEQ ID NOs: 1-159 (for SaCas9, including SaCas9KKH), and 200-292, 924-938, or 950-955 (for SluCas9). In some embodiments, the guide sequence comprises a sequence selected from SEQ ID NOs: 1-159, 200-292, 924-938, or 950-955. In some embodiments, the target sequence is in a gene or on a chromosome, for example, and is complementary to the guide sequence. In some embodiments, the degree of complementarity or identity between a guide sequence and its corresponding target sequence may be about 75%, 80%, 85%, 90%, 95%, 96%, 97%, 98%, 99%, or 100%. For example, in some embodiments, the guide sequence comprises a sequence with about 75%, 80%, 85%, 90%, 95%, 96%, 97%, 98%, 99%, or 100% identity to at least 16, 17, 18, 19, 20, 21, 22, 23, 24, or 25 contiguous nucleotides of a sequence selected from SEQ ID NOs: 1-159, 200-292, 924-938, or 950-955. In some embodiments, the guide sequence comprises a sequence with about 75%, 80%, 85%, 90%, 95%, 96%, 97%, 98%, 99%, or 100% identity to a sequence selected from SEQ ID NOs: 1-159, 200-292, 924-938, or 950-955. In preferred embodiments, a guide/spacer sequence in the case of SluCas9 or SaCas9 is at least 20 base pairs in length, or more specifically, within 20-25 base pairs in length (see, e.g., Schmidt et al., 2021, Nature Communications, “Improved CRISPR genome editing using small highly active and specific engineered RNA-guided nucleases”). In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 16, 17, 18, 19, 20 or more base pairs. In some embodiments, the total length of the target sequence/guide is no more than 16, no more than 17, no more than 18, no more than 19, no more than 20, no more than 22, no more than 23, or no more than 24 nucleotides in length. In some embodiments, the guide sequence and the target region may contain 1-4 mismatches where the guide sequence comprises at least 16, 17, 18, 19, 20 or more nucleotides. In some embodiments, the guide sequence and the target region may contain 1, 2, 3, or 4 mismatches where the guide sequence comprises 20 nucleotides. In some embodiments, the guide sequence and the target region do not contain any mismatches.

In some embodiments, the guide sequence comprises a sequence selected from SEQ ID NOs: 1-159, 200-292, 924-938, or 950-955, wherein if the 5′ terminal nucleotide is not guanine, one or more guanine (g) is added to the sequence at its 5′ end. In some embodiments, the 5′ g or gg is included in some instances for transcription, for example, for expression by the RNA polymerase III-dependent U6 promoter or the T7 promoter. In some embodiments, a 5′ guanine is added to any one of the guide sequences or pairs of guide sequences disclosed herein.

Target sequences for Cas9s include both the positive and negative strands of genomic DNA (i.e., the sequence given and the sequence's reverse compliment), as a nucleic acid substrate for a Cas9 is a double stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, “ribonucleoprotein” (RNP) or “RNP complex” refers to a guide RNA together with a Cas9. In some embodiments, the guide RNA guides the Cas9, such as a SluCas9 or a SaCas9, to a target sequence, and the guide RNA hybridizes with and the agent binds to the target sequence, which can be followed by cleaving or nicking (in the context of a modified “nickase” Cas9).

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity>50% for amino acids or >75% for nucleotides, the Needleman-Wunsch algorithm with default settings of the Needleman-Wunsch algorithm interface provided by the EBI at the www.ebi.ac.uk web server is generally appropriate. [0033]“mRNA” is used herein to refer to a polynucleotide that is not DNA and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof.

Guide sequences useful in the guide RNA compositions and methods described herein are shown, for example, in Tables 1-6, and throughout the specification.

As used herein, a “target sequence” refers to a sequence of nucleic acid in a target gene that has complementarity to at least a portion of the guide sequence of the guide RNA. The interaction of the target sequence and the guide sequence directs a Cas9 to bind, and potentially nick or cleave (depending on the activity of the agent), within or near the target sequence.

As used herein, “treatment” refers to any administration or application of a therapeutic for disease or disorder in a subject, and includes inhibiting the disease or development of the disease (which may occur before or after the disease is formally diagnosed, e.g., in cases where a subject has a genotype that has the potential or is likely to result in development of the disease), arresting its development, relieving one or more symptoms of the disease, curing the disease, or preventing reoccurrence of one or more symptoms of the disease. For example, treatment of DMD may comprise alleviating symptoms of DMD.

As used herein, “ameliorating” refers to any beneficial effect on a phenotype or symptom, such as reducing its severity, slowing or delaying its development, arresting its development, or partially or completely reversing or eliminating it. In the case of quantitative phenotypes such as expression levels, ameliorating encompasses changing the expression level so that it is closer to the expression level seen in healthy or unaffected cells or individuals.

A “pharmaceutically acceptable excipient” refers to an agent that is included in a pharmaceutical formulation that is not the active ingredient. Pharmaceutically acceptable excipients may e.g., aid in drug delivery or support or enhance stability or bioavailability.

The term “about” or “approximately” means an acceptable error for a particular value as determined by one of ordinary skill in the art, which depends in part on how the value is measured or determined.

As used herein, “Staphylococcus aureus Cas9” may also be referred to as SaCas9, and includes wild type SaCas9 (e.g., SEQ ID NO: 711) and variants thereof. A variant of SaCas9 comprises one or more amino acid changes as compared to SEQ ID NO: 711, including insertion, deletion, or substitution of one or more amino acids, or a chemical modification to one or more amino acids. For clarity, SaCas9KKH is a SaCas9 variant.

As used herein, “Staphylococcus lugdunensis Cas9” may also be referred to as SluCas9, and includes wild type SluCas9 (e.g., SEQ ID NO: 712) and variants thereof. A variant of SluCas9 comprises one or more amino acid changes as compared to SEQ ID NO: 712, including insertion, deletion, or substitution of one or more amino acids, or a chemical modification to one or more amino acids.

### II. Compositions

Provided herein are compositions comprising guide RNAs and pairs of guide RNAs useful for treating Duchenne Muscular Dystrophy (DMD). The provided pairs of guide RNAs, when used with the correct endonuclease, function to precisely delete a small portion (e.g., less than about 250 nucleotides) of exon 44, 45, 50, 51, or 53 of the DMD gene. Table 6 provides a listing of guide sequences of guide RNAs, and Tables 1A-5D) provide detailed information regarding these sequences.

### Guides and Guide Pairs

### a) Exon 44:

TABLE 1A
(1) Exon 44 SaCas9 guides:

| Seq | | |
|---|---|---|
| ID | Guide ID or Guide | |
| No. | RNA Name | Guide Sequence |
| 1 | E44Sa1 | ATTTAGCATGTTCCCAATTCTC |
| 2 | E44Sa3 | TCTCAGAAAGACACAAATTCCT |
| 104 | E44SaCas9KKH2 | TACCATTTGTATTTAGCATGTT |
| 105 | E44SaCas9KKH3 | ATTTAGCATGTTCCCAATTCTC |
| 106 | E44SaCas9KKH4 | AACTGTTCAGCTTCTGTTAGCC |
| 107 | E44SaCas9KKH5 | TTCAGCTTCTGTTAGCCACTGA |
| 108 | E44SaCas9KKH6 | CACTGATTAAATATCTTTATAT |
| 109 | E44SaCas9KKH7 | ATGAAAACGCCGCCATTTCTCA |
| 110 | E44SaCas9KKH8 | CCGCCATTTCTCAACAGATCTG |
| 111 | E44SaCas9KKH9 | CAACAGATCTGTCAAATCGCCT |
| 112 | E44SaCas9KKH14 | TAAATACAAATGGTATCTTAAG |
| 113 | E44SaCas9KKH15 | ATGCTAAATACAAATGGTATCT |
| 114 | E44SaCas9KKH16 | AATTGGGAACATGCTAAATACA |
| 115 | E44SaCas9KKH17 | GAGAATTGGGAACATGCTAAAT |
| 116 | E44SaCas9KKH18 | ATTCCTGAGAATTGGGAACATG |
| 117 | E44SaCas9KKH19 | TCTCAGAAAGACACAAATTCCT |
| 118 | E44SaCas9KKH20 | CTGAACAGTTTCTCAGAAAGAC |
| 119 | E44SaCas9KKH21 | AATCAGTGGCTAACAGAAGCTG |
| 120 | E44SaCas9KKH22 | CATTATGATATAAAGATATTTA |
| 121 | E44SaCas9KKH23 | TTTTCATTATGATATAAAGATA |
| 122 | E44SaCas9KKH24 | GGCGGCGTTTTCATTATGATAT |
| 123 | E44SaCas9KKH25 | TGAGAAATGGCGGCGTTTTCAT |
| 124 | E44SaCas9KKH26 | AGGCGATTTGACAGATCTGTTG |
| 125 | E44SaCas9KKH27 | GCTTTTACCTGCAGGCGATTTG |

TABLE 1B
(2) Exon 44 SaCas9 pairs:

| Guide 1 Seq | | | Guide 2 Seq | | |
|---|---|---|---|---|---|
| ID No. | Guide 1 | Guide 1 Sequence | ID No. | Guide 2 | Guide 2 Sequence |
| 1 | E44Sa1 | ATTTAGCATGTTCCCA | 2 | E44Sa3 | TCTCAGAAAGACACAA |
| | | ATTCTC | | | ATTCCT |
| 110 | E44SaCas9KKH8 | CCGCCATTTCTCAACA | 120 | E44SaCas9KKH22 | CATTATGATATAAAGA |
| | | GATCTG | | | TATTTA |
| 110 | E44SaCas9KKH8 | CCGCCATTTCTCAACA | 121 | E44SaCas9KKH23 | TTTTCATTATGATATA |
| | | GATCTG | | | AAGATA |
| 110 | E44SaCas9KKH8 | CCGCCATTTCTCAACA | 122 | E44SaCas9KKH24 | GGCGGCGTTTTCATTA |
| | | GATCTG | | | TGATAT |
| 110 | E44SaCas9KKH8 | CCGCCATTTCTCAACA | 123 | E44SaCas9KKH25 | TGAGAAATGGCGGCGT |
| | | GATCTG | | | TTTCAT |
| 110 | E44SaCas9KKH8 | CCGCCATTTCTCAACA | 124 | E44SaCas9KKH26 | AGGCGATTTGACAGAT |
| | | GATCTG | | | CTGTTG |
| 110 | E44SaCas9KKH8 | CCGCCATTTCTCAACA | 125 | E44SaCas9KKH27 | GCTTTTACCTGCAGGC |
| | | GATCTG | | | GATTTG |
| 111 | E44SaCas9KKH9 | CAACAGATCTGTCAAA | 112 | E44SaCas9KKH14 | TAAATACAAATGGTAT |
| | | TCGCCT | | | CTTAAG |
| 111 | E44SaCas9KKH9 | CAACAGATCTGTCAAA | 113 | E44SaCas9KKH15 | ATGCTAAATACAAATG |
| | | TCGCCT | | | GTATCT |
| 111 | E44SaCas9KKH9 | CAACAGATCTGTCAAA | 114 | E44SaCas9KKH16 | AATTGGGAACATGCTA |
| | | TCGCCT | | | AATACA |
| 111 | E44SaCas9KKH9 | CAACAGATCTGTCAAA | 115 | E44SaCas9KKH17 | GAGAATTGGGAACATG |
| | | TCGCCT | | | CTAAAT |
| 111 | E44SaCas9KKH9 | CAACAGATCTGTCAAA | 116 | E44SaCas9KKH18 | ATTCCTGAGAATTGGG |
| | | TCGCCT | | | AACATG |
| 111 | E44SaCas9KKH9 | CAACAGATCTGTCAAA | 117 | E44SaCas9KKH19 | TCTCAGAAAGACACAA |
| | | TCGCCT | | | ATTCCT |
| 111 | E44SaCas9KKH9 | CAACAGATCTGTCAAA | 118 | E44SaCas9KKH20 | CTGAACAGTTTCTCAG |
| | | TCGCCT | | | AAAGAC |
| 111 | E44SaCas9KKH9 | CAACAGATCTGTCAAA | 119 | E44SaCas9KKH21 | AATCAGTGGCTAACAG |
| | | TCGCCT | | | AAGCTG |
| 111 | E44SaCas9KKH9 | CAACAGATCTGTCAAA | 120 | E44SaCas9KKH22 | CATTATGATATAAAGA |
| | | TCGCCT | | | TATTTA |
| 111 | E44SaCas9KKH9 | CAACAGATCTGTCAAA | 121 | E44SaCas9KKH23 | TTTTCATTATGATATA |
| | | TCGCCT | | | AAGATA |
| 111 | E44SaCas9KKH9 | CAACAGATCTGTCAAA | 122 | E44SaCas9KKH24 | GGCGGCGTTTTCATTA |
| | | TCGCCT | | | TGATAT |
| 111 | E44SaCas9KKH9 | CAACAGATCTGTCAAA | 123 | E44SaCas9KKH25 | TGAGAAATGGCGGCGT |
| | | TCGCCT | | | TTTCAT |
| 111 | E44SaCas9KKH9 | CAACAGATCTGTCAAA | 124 | E44SaCas9KKH26 | AGGCGATTTGACAGAT |
| | | TCGCCT | | | CTGTTG |
| 111 | E44SaCas9KKH9 | CAACAGATCTGTCAAA | 125 | E44SaCas9KKH27 | GCTTTTACCTGCAGGC |
| | | TCGCCT | | | GATTTG |
| 112 | E44SaCas9KKH14 | TAAATACAAATGGTAT | 113 | E44SaCas9KKH15 | ATGCTAAATACAAATG |
| | | CTTAAG | | | GTATCT |
| 112 | E44SaCas9KKH14 | TAAATACAAATGGTAT | 114 | E44SaCas9KKH16 | AATTGGGAACATGCTA |
| | | CTTAAG | | | AATACA |
| 112 | E44SaCas9KKH14 | TAAATACAAATGGTAT | 115 | E44SaCas9KKH17 | GAGAATTGGGAACATG |
| | | CTTAAG | | | CTAAAT |
| 112 | E44SaCas9KKH14 | TAAATACAAATGGTAT | 116 | E44SaCas9KKH18 | ATTCCTGAGAATTGGG |
| | | CTTAAG | | | AACATG |
| 112 | E44SaCas9KKH14 | TAAATACAAATGGTAT | 117 | E44SaCas9KKH19 | TCTCAGAAAGACACAA |
| | | CTTAAG | | | ATTCCT |
| 112 | E44SaCas9KKH14 | TAAATACAAATGGTAT | 118 | E44SaCas9KKH20 | CTGAACAGTTTCTCAG |
| | | CTTAAG | | | AAAGAC |
| 112 | E44SaCas9KKH14 | TAAATACAAATGGTAT | 119 | E44SaCas9KKH21 | AATCAGTGGCTAACAG |
| | | CTTAAG | | | AAGCTG |
| 112 | E44SaCas9KKH14 | TAAATACAAATGGTAT | 120 | E44SaCas9KKH22 | CATTATGATATAAAGA |
| | | CTTAAG | | | TATTTA |
| 112 | E44SaCas9KKH14 | TAAATACAAATGGTAT | 121 | E44SaCas9KKH23 | TTTTCATTATGATATA |
| | | CTTAAG | | | AAGATA |
| 112 | E44SaCas9KKH14 | TAAATACAAATGGTAT | 122 | E44SaCas9KKH24 | GGCGGCGTTTTCATTA |
| | | CTTAAG | | | TGATAT |
| 112 | E44SaCas9KKH14 | TAAATACAAATGGTAT | 123 | E44SaCas9KKH25 | TGAGAAATGGCGGCGT |
| | | CTTAAG | | | TTTCAT |
| 112 | E44SaCas9KKH14 | TAAATACAAATGGTAT | 124 | E44SaCas9KKH26 | AGGCGATTTGACAGAT |
| | | CTTAAG | | | CTGTTG |
| 112 | E44SaCas9KKH14 | TAAATACAAATGGTAT | 125 | E44SaCas9KKH27 | GCTTTTACCTGCAGGC |
| | | CTTAAG | | | GATTTG |
| 113 | E44SaCas9KKH15 | ATGCTAAATACAAATG | 114 | E44SaCas9KKH16 | AATTGGGAACATGCTA |
| | | GTATCT | | | AATACA |
| 113 | E44SaCas9KKH15 | ATGCTAAATACAAATG | 115 | E44SaCas9KKH17 | GAGAATTGGGAACATG |
| | | GTATCT | | | CTAAAT |
| 113 | E44SaCas9KKH15 | ATGCTAAATACAAATG | 116 | E44SaCas9KKH18 | ATTCCTGAGAATTGGG |
| | | GTATCT | | | AACATG |
| 113 | E44SaCas9KKH15 | ATGCTAAATACAAATG | 117 | E44SaCas9KKH19 | TCTCAGAAAGACACAA |
| | | GTATCT | | | ATTCCT |
| 113 | E44SaCas9KKH15 | ATGCTAAATACAAATG | 118 | E44SaCas9KKH20 | CTGAACAGTTTCTCAG |
| | | GTATCT | | | AAAGAC |
| 113 | E44SaCas9KKH15 | ATGCTAAATACAAATG | 119 | E44SaCas9KKH21 | AATCAGTGGCTAACAG |
| | | GTATCT | | | AAGCTG |
| 113 | E44SaCas9KKH15 | ATGCTAAATACAAATG | 120 | E44SaCas9KKH22 | CATTATGATATAAAGA |
| | | GTATCT | | | TATTTA |
| 113 | E44SaCas9KKH15 | ATGCTAAATACAAATG | 121 | E44SaCas9KKH23 | TTTTCATTATGATATA |
| | | GTATCT | | | AAGATA |
| 113 | E44SaCas9KKH15 | ATGCTAAATACAAATG | 122 | E44SaCas9KKH24 | GGCGGCGTTTTCATTA |
| | | GTATCT | | | TGATAT |
| 113 | E44SaCas9KKH15 | ATGCTAAATACAAATG | 123 | E44SaCas9KKH25 | TGAGAAATGGCGGCGT |
| | | GTATCT | | | TTTCAT |
| 113 | E44SaCas9KKH15 | ATGCTAAATACAAATG | 124 | E44SaCas9KKH26 | AGGCGATTTGACAGAT |
| | | GTATCT | | | CTGTTG |
| 113 | E44SaCas9KKH15 | ATGCTAAATACAAATG | 125 | E44SaCas9KKH27 | GCTTTTACCTGCAGGC |
| | | GTATCT | | | GATTTG |
| 114 | E44SaCas9KKH16 | AATTGGGAACATGCTA | 115 | E44SaCas9KKH17 | GAGAATTGGGAACATG |
| | | AATACA | | | CTAAAT |
| 114 | E44SaCas9KKH16 | AATTGGGAACATGCTA | 116 | E44SaCas9KKH18 | ATTCCTGAGAATTGGG |
| | | AATACA | | | AACATG |
| 114 | E44SaCas9KKH16 | AATTGGGAACATGCTA | 117 | E44SaCas9KKH19 | TCTCAGAAAGACACAA |
| | | AATACA | | | ATTCCT |
| 114 | E44SaCas9KKH16 | AATTGGGAACATGCTA | 118 | E44SaCas9KKH20 | CTGAACAGTTTCTCAG |
| | | AATACA | | | AAAGAC |
| 114 | E44SaCas9KKH16 | AATTGGGAACATGCTA | 119 | E44SaCas9KKH21 | AATCAGTGGCTAACAG |
| | | AATACA | | | AAGCTG |
| 114 | E44SaCas9KKH16 | AATTGGGAACATGCTA | 120 | E44SaCas9KKH22 | CATTATGATATAAAGA |
| | | AATACA | | | TATTTA |
| 114 | E44SaCas9KKH16 | AATTGGGAACATGCTA | 121 | E44SaCas9KKH23 | TTTTCATTATGATATA |
| | | AATACA | | | AAGATA |
| 114 | E44SaCas9KKH16 | AATTGGGAACATGCTA | 122 | E44SaCas9KKH24 | GGCGGCGTTTTCATTA |
| | | AATACA | | | TGATAT |
| 114 | E44SaCas9KKH16 | AATTGGGAACATGCTA | 123 | E44SaCas9KKH25 | TGAGAAATGGCGGCGT |
| | | AATACA | | | TTTCAT |
| 114 | E44SaCas9KKH16 | AATTGGGAACATGCTA | 124 | E44SaCas9KKH26 | AGGCGATTTGACAGAT |
| | | AATACA | | | CTGTTG |
| 114 | E44SaCas9KKH16 | AATTGGGAACATGCTA | 125 | E44SaCas9KKH27 | GCTTTTACCTGCAGGC |
| | | AATACA | | | GATTTG |
| 115 | E44SaCas9KKH17 | GAGAATTGGGAACATG | 116 | E44SaCas9KKH18 | ATTCCTGAGAATTGGG |
| | | CTAAAT | | | AACATG |
| 115 | E44SaCas9KKH17 | GAGAATTGGGAACATG | 117 | E44SaCas9KKH19 | TCTCAGAAAGACACAA |
| | | CTAAAT | | | ATTCCT |
| 115 | E44SaCas9KKH17 | GAGAATTGGGAACATG | 118 | E44SaCas9KKH20 | CTGAACAGTTTCTCAG |
| | | CTAAAT | | | AAAGAC |
| 115 | E44SaCas9KKH17 | GAGAATTGGGAACATG | 119 | E44SaCas9KKH21 | AATCAGTGGCTAACAG |
| | | CTAAAT | | | AAGCTG |
| 115 | E44SaCas9KKH17 | GAGAATTGGGAACATG | 120 | E44SaCas9KKH22 | CATTATGATATAAAGA |
| | | CTAAAT | | | TATTTA |
| 115 | E44SaCas9KKH17 | GAGAATTGGGAACATG | 121 | E44SaCas9KKH23 | TTTTCATTATGATATA |
| | | CTAAAT | | | AAGATA |
| 115 | E44SaCas9KKH17 | GAGAATTGGGAACATG | 122 | E44SaCas9KKH24 | GGCGGCGTTTTCATTA |
| | | CTAAAT | | | TGATAT |
| 115 | E44SaCas9KKH17 | GAGAATTGGGAACATG | 123 | E44SaCas9KKH25 | TGAGAAATGGCGGCGT |
| | | CTAAAT | | | TTTCAT |
| 115 | E44SaCas9KKH17 | GAGAATTGGGAACATG | 124 | E44SaCas9KKH26 | AGGCGATTTGACAGAT |
| | | CTAAAT | | | CTGTTG |
| 115 | E44SaCas9KKH17 | GAGAATTGGGAACATG | 125 | E44SaCas9KKH27 | GCTTTTACCTGCAGGC |
| | | CTAAAT | | | GATTTG |
| 116 | E44SaCas9KKH18 | ATTCCTGAGAATTGGG | 117 | E44SaCas9KKH19 | TCTCAGAAAGACACAA |
| | | AACATG | | | ATTCCT |
| 116 | E44SaCas9KKH18 | ATTCCTGAGAATTGGG | 118 | E44SaCas9KKH20 | CTGAACAGTTTCTCAG |
| | | AACATG | | | AAAGAC |
| 116 | E44SaCas9KKH18 | ATTCCTGAGAATTGGG | 119 | E44SaCas9KKH21 | AATCAGTGGCTAACAG |
| | | AACATG | | | AAGCTG |
| 116 | E44SaCas9KKH18 | ATTCCTGAGAATTGGG | 120 | E44SaCas9KKH22 | CATTATGATATAAAGA |
| | | AACATG | | | TATTTA |
| 116 | E44SaCas9KKH18 | ATTCCTGAGAATTGGG | 121 | E44SaCas9KKH23 | TTTTCATTATGATATA |
| | | AACATG | | | AAGATA |
| 116 | E44SaCas9KKH18 | ATTCCTGAGAATTGGG | 122 | E44SaCas9KKH24 | GGCGGCGTTTTCATTA |
| | | AACATG | | | TGATAT |
| 116 | E44SaCas9KKH18 | ATTCCTGAGAATTGGG | 123 | E44SaCas9KKH25 | TGAGAAATGGCGGCGT |
| | | AACATG | | | TTTCAT |
| 116 | E44SaCas9KKH18 | ATTCCTGAGAATTGGG | 124 | E44SaCas9KKH26 | AGGCGATTTGACAGAT |
| | | AACATG | | | CTGTTG |
| 116 | E44SaCas9KKH18 | ATTCCTGAGAATTGGG | 125 | E44SaCas9KKH27 | GCTTTTACCTGCAGGC |
| | | AACATG | | | GATTTG |
| 117 | E44SaCas9KKH19 | TCTCAGAAAGACACAA | 118 | E44SaCas9KKH20 | CTGAACAGTTTCTCAG |
| | | ATTCCT | | | AAAGAC |
| 117 | E44SaCas9KKH19 | TCTCAGAAAGACACAA | 119 | E44SaCas9KKH21 | AATCAGTGGCTAACAG |
| | | ATTCCT | | | AAGCTG |
| 117 | E44SaCas9KKH19 | TCTCAGAAAGACACAA | 120 | E44SaCas9KKH22 | CATTATGATATAAAGA |
| | | ATTCCT | | | TATTTA |
| 117 | E44SaCas9KKH19 | TCTCAGAAAGACACAA | 121 | E44SaCas9KKH23 | TTTTCATTATGATATA |
| | | ATTCCT | | | AAGATA |
| 117 | E44SaCas9KKH19 | TCTCAGAAAGACACAA | 122 | E44SaCas9KKH24 | GGCGGCGTTTTCATTA |
| | | ATTCCT | | | TGATAT |
| 117 | E44SaCas9KKH19 | TCTCAGAAAGACACAA | 123 | E44SaCas9KKH25 | TGAGAAATGGCGGCGT |
| | | ATTCCT | | | TTTCAT |
| 117 | E44SaCas9KKH19 | TCTCAGAAAGACACAA | 124 | E44SaCas9KKH26 | AGGCGATTTGACAGAT |
| | | ATTCCT | | | CTGTTG |
| 117 | E44SaCas9KKH19 | TCTCAGAAAGACACAA | 125 | E44SaCas9KKH27 | GCTTTTACCTGCAGGC |
| | | ATTCCT | | | GATTTG |
| 118 | E44SaCas9KKH20 | CTGAACAGTTTCTCAG | 119 | E44SaCas9KKH21 | AATCAGTGGCTAACAG |
| | | AAAGAC | | | AAGCTG |
| 118 | E44SaCas9KKH20 | CTGAACAGTTTCTCAG | 120 | E44SaCas9KKH22 | CATTATGATATAAAGA |
| | | AAAGAC | | | TATTTA |
| 118 | E44SaCas9KKH20 | CTGAACAGTTTCTCAG | 121 | E44SaCas9KKH23 | TTTTCATTATGATATA |
| | | AAAGAC | | | AAGATA |
| 118 | E44SaCas9KKH20 | CTGAACAGTTTCTCAG | 122 | E44SaCas9KKH24 | GGCGGCGTTTTCATTA |
| | | AAAGAC | | | TGATAT |
| 118 | E44SaCas9KKH20 | CTGAACAGTTTCTCAG | 123 | E44SaCas9KKH25 | TGAGAAATGGCGGCGT |
| | | AAAGAC | | | TTTCAT |
| 118 | E44SaCas9KKH20 | CTGAACAGTTTCTCAG | 124 | E44SaCas9KKH26 | AGGCGATTTGACAGAT |
| | | AAAGAC | | | CTGTTG |
| 118 | E44SaCas9KKH20 | CTGAACAGTTTCTCAG | 125 | E44SaCas9KKH27 | GCTTTTACCTGCAGGC |
| | | AAAGAC | | | GATTTG |
| 119 | E44SaCas9KKH21 | AATCAGTGGCTAACAG | 120 | E44SaCas9KKH22 | CATTATGATATAAAGA |
| | | AAGCTG | | | TATTTA |
| 119 | E44SaCas9KKH21 | AATCAGTGGCTAACAG | 121 | E44SaCas9KKH23 | TTTTCATTATGATATA |
| | | AAGCTG | | | AAGATA |
| 119 | E44SaCas9KKH21 | AATCAGTGGCTAACAG | 122 | E44SaCas9KKH24 | GGCGGCGTTTTCATTA |
| | | AAGCTG | | | TGATAT |
| 119 | E44SaCas9KKH21 | AATCAGTGGCTAACAG | 123 | E44SaCas9KKH25 | TGAGAAATGGCGGCGT |
| | | AAGCTG | | | TTTCAT |
| 119 | E44SaCas9KKH21 | AATCAGTGGCTAACAG | 124 | E44SaCas9KKH26 | AGGCGATTTGACAGAT |
| | | AAGCTG | | | CTGTTG |
| 119 | E44SaCas9KKH21 | AATCAGTGGCTAACAG | 125 | E44SaCas9KKH27 | GCTTTTACCTGCAGGC |
| | | AAGCTG | | | GATTTG |
| 120 | E44SaCas9KKH22 | CATTATGATATAAAGA | 121 | E44SaCas9KKH23 | TTTTCATTATGATATA |
| | | TATTTA | | | AAGATA |
| 120 | E44SaCas9KKH22 | CATTATGATATAAAGA | 122 | E44SaCas9KKH24 | GGCGGCGTTTTCATTA |
| | | TATTTA | | | TGATAT |
| 120 | E44SaCas9KKH22 | CATTATGATATAAAGA | 123 | E44SaCas9KKH25 | TGAGAAATGGCGGCGT |
| | | TATTTA | | | TTTCAT |
| 120 | E44SaCas9KKH22 | CATTATGATATAAAGA | 124 | E44SaCas9KKH26 | AGGCGATTTGACAGAT |
| | | TATTTA | | | CTGTTG |
| 120 | E44SaCas9KKH22 | CATTATGATATAAAGA | 125 | E44SaCas9KKH27 | GCTTTTACCTGCAGGC |
| | | TATTTA | | | GATTTG |
| 121 | E44SaCas9KKH23 | TTTTCATTATGATATA | 122 | E44SaCas9KKH24 | GGCGGCGTTTTCATTA |
| | | AAGATA | | | TGATAT |
| 121 | E44SaCas9KKH23 | TTTTCATTATGATATA | 123 | E44SaCas9KKH25 | TGAGAAATGGCGGCGT |
| | | AAGATA | | | TTTCAT |
| 121 | E44SaCas9KKH23 | TTTTCATTATGATATA | 124 | E44SaCas9KKH26 | AGGCGATTTGACAGAT |
| | | AAGATA | | | CTGTTG |
| 121 | E44SaCas9KKH23 | TTTTCATTATGATATA | 125 | E44SaCas9KKH27 | GCTTTTACCTGCAGGC |
| | | AAGATA | | | GATTTG |
| 122 | E44SaCas9KKH24 | GGCGGCGTTTTCATTA | 123 | E44SaCas9KKH25 | TGAGAAATGGCGGCGT |
| | | TGATAT | | | TTTCAT |
| 122 | E44SaCas9KKH24 | GGCGGCGTTTTCATTA | 124 | E44SaCas9KKH26 | AGGCGATTTGACAGAT |
| | | TGATAT | | | CTGTTG |
| 122 | E44SaCas9KKH24 | GGCGGCGTTTTCATTA | 125 | E44SaCas9KKH27 | GCTTTTACCTGCAGGC |
| | | TGATAT | | | GATTTG |
| 123 | E44SaCas9KKH25 | TGAGAAATGGCGGCGT | 124 | E44SaCas9KKH26 | AGGCGATTTGACAGAT |
| | | TTTCAT | | | CTGTTG |
| 123 | E44SaCas9KKH25 | TGAGAAATGGCGGCGT | 125 | E44SaCas9KKH27 | GCTTTTACCTGCAGGC |
| | | TTTCAT | | | GATTTG |
| 124 | E44SaCas9KKH26 | AGGCGATTTGACAGAT | 125 | E44SaCas9KKH27 | GCTTTTACCTGCAGGC |
| | | CTGTTG | | | GATTTG |

TABLE 1C
(3) Exon 44 SluCas9 guides:

| Seq | | |
|---|---|---|
| ID | Guide ID or Guide | |
| No. | RNA Name | Guide Sequence |
| 200 | E44Slu1 | TATTTAGCATGTTCCCAATTCT |
| 201 | E44Slu2 | AACAGATCTGTCAAATCGCCTG |
| 202 | E44Slu4 | TGCTAAATACAAATGGTATCTT |
| 203 | E44Slu5 | ATTGGGAACATGCTAAATACAA |
| 204 | E44Slu6 | AAAGACACAAATTCCTGAGAAT |
| 205 | E44Slu7 | GAAAGACACAAATTCCTGAGAA |
| 206 | E44Slu8 | ATGATATAAAGATATTTAATCA |
| 207 | E44Slu9 | TTGACAGATCTGTTGAGAAATG |
| 208 | E44Slu10 | GATTTGACAGATCTGTTGAGAA |

TABLE 1D
(4) Exon 44 SluCas9 pairs:

| Guide 1 Seq ID No. | Guide 1 | Guide 1 Sequence | Guide 2 Seq ID No. | Guide 2 | Guide 2 Sequence |
|---|---|---|---|---|---|
| 200 | E44SL1 | TATTTAGCATGTTCCCAATTCT | 201 | E44SL2 | AACAGATCTGTCAAATCGCCTG |
| 200 | E44SL1 | TATTTAGCATGTTCCCAATTCT | 202 | E44SL4 | TGCTAAATACAAATGGTATCTT |
| 200 | E44SL1 | TATTTAGCATGTTCCCAATTCT | 203 | E44SL5 | ATTGGGAACATGCTAAATACAA |
| 200 | E44SL1 | TATTTAGCATGTTCCCAATTCT | 204 | E44SL6 | AAAGACACAAATTCCTGAGAAT |
| 200 | E44SL1 | TATTTAGCATGTTCCCAATTCT | 205 | E44SL7 | GAAAGACACAAATTCCTGAGAA |
| 200 | E44SL1 | TATTTAGCATGTTCCCAATTCT | 206 | E44SL8 | ATGATATAAAGATATTTAATCA |
| 200 | E44SL1 | TATTTAGCATGTTCCCAATTCT | 207 | E44SL9 | TTGACAGATCTGTTGAGAAATG |
| 200 | E44SL1 | TATTTAGCATGTTCCCAATTCT | 208 | E44SL10 | GATTTGACAGATCTGTTGAGAA |
| 201 | E44SL2 | AACAGATCTGTCAAATCGCCTG | 202 | E44SL4 | TGCTAAATACAAATGGTATCTT |
| 201 | E44SL2 | AACAGATCTGTCAAATCGCCTG | 203 | E44SL5 | ATTGGGAACATGCTAAATACAA |
| 201 | E44SL2 | AACAGATCTGTCAAATCGCCTG | 204 | E44SL6 | AAAGACACAAATTCCTGAGAAT |
| 201 | E44SL2 | AACAGATCTGTCAAATCGCCTG | 205 | E44SL7 | GAAAGACACAAATTCCTGAGAA |
| 201 | E44SL2 | AACAGATCTGTCAAATCGCCTG | 206 | E44SL8 | ATGATATAAAGATATTTAATCA |
| 201 | E44SL2 | AACAGATCTGTCAAATCGCCTG | 207 | E44SL9 | TTGACAGATCTGTTGAGAAATG |
| 201 | E44SL2 | AACAGATCTGTCAAATCGCCTG | 208 | E44SL10 | GATTTGACAGATCTGTTGAGAA |
| 202 | E44SL4 | TGCTAAATACAAATGGTATCTT | 203 | E44SL5 | ATTGGGAACATGCTAAATACAA |
| 202 | E44SL4 | TGCTAAATACAAATGGTATCTT | 204 | E44SL6 | AAAGACACAAATTCCTGAGAAT |
| 202 | E44SL4 | TGCTAAATACAAATGGTATCTT | 205 | E44SL7 | GAAAGACACAAATTCCTGAGAA |
| 202 | E44SL4 | TGCTAAATACAAATGGTATCTT | 206 | E44SL8 | ATGATATAAAGATATTTAATCA |
| 202 | E44SL4 | TGCTAAATACAAATGGTATCTT | 207 | E44SL9 | TTGACAGATCTGTTGAGAAATG |
| 202 | E44SL4 | TGCTAAATACAAATGGTATCTT | 208 | E44SL10 | GATTTGACAGATCTGTTGAGAA |
| 203 | E44SL5 | ATTGGGAACATGCTAAATACAA | 204 | E44SL6 | AAAGACACAAATTCCTGAGAAT |
| 203 | E44SL5 | ATTGGGAACATGCTAAATACAA | 205 | E44SL7 | GAAAGACACAAATTCCTGAGAA |
| 203 | E44SL5 | ATTGGGAACATGCTAAATACAA | 206 | E44SL8 | ATGATATAAAGATATTTAATCA |
| 203 | E44SL5 | ATTGGGAACATGCTAAATACAA | 207 | E44SL9 | TTGACAGATCTGTTGAGAAATG |
| 203 | E44SL5 | ATTGGGAACATGCTAAATACAA | 208 | E44SL10 | GATTTGACAGATCTGTTGAGAA |
| 204 | E44SL6 | AAAGACACAAATTCCTGAGAAT | 205 | E44SL7 | GAAAGACACAAATTCCTGAGAA |
| 204 | E44SL6 | AAAGACACAAATTCCTGAGAAT | 206 | E44SL8 | ATGATATAAAGATATTTAATCA |
| 204 | E44SL6 | AAAGACACAAATTCCTGAGAAT | 207 | E44SL9 | TTGACAGATCTGTTGAGAAATG |
| 204 | E44SL6 | AAAGACACAAATTCCTGAGAAT | 208 | E44SL10 | GATTTGACAGATCTGTTGAGAA |
| 205 | E44SL7 | GAAAGACACAAATTCCTGAGAA | 206 | E44SL8 | ATGATATAAAGATATTTAATCA |
| 205 | E44SL7 | GAAAGACACAAATTCCTGAGAA | 207 | E44SL9 | TTGACAGATCTGTTGAGAAATG |
| 205 | E44SL7 | GAAAGACACAAATTCCTGAGAA | 208 | E44SL10 | GATTTGACAGATCTGTTGAGAA |
| 206 | E44SL8 | ATGATATAAAGATATTTAATCA | 207 | E44SL9 | TTGACAGATCTGTTGAGAAATG |
| 206 | E44SL8 | ATGATATAAAGATATTTAATCA | 208 | E44SL10 | GATTTGACAGATCTGTTGAGAA |
| 207 | E44SL9 | TTGACAGATCTGTTGAGAAATG | 208 | E44SL10 | GATTTGACAGATCTGTTGAGAA |

### b) Exon 45:

TABLE 2A
(1) Exon 45 SaCas9 guides:

| Seq | | |
|---|---|---|
| ID | Guide ID or Guide | |
| No. | RNA Name | Guide Sequence |
| 3 | E45Sa1 | TCAGGCTTCCCAATTTTTCCTG |
| 4 | E45Sa2 | TAGAATACTGGCATCTGTTTTT |
| 5 | E45Sa3 | TGGCATCTGTTTTTGAGGATTG |
| 6 | E45Sa4 | TTGCCGCTGCCCAATGCCATCC |
| 7 | E45Sa6 | TCTACAGGAAAAATTGGGAAGC |
| 8 | E45Sa7 | GCGGCAAACTGTTGTCAGAACA |
| 9 | E45Sa8 | TTTTGGTATCTTACAGGAACTC |
| 126 | E45SaCas9KKH1 | TGTTTGCAGACCTCCTGCCACC |
| 127 | E45SaCas9KKH2 | CTGCCACCGCAGATTCAGGCTT |
| 128 | E45SaCas9KKH3 | TCAGGCTTCCCAATTTTTCCTG |
| 129 | E45SaCas9KKH4 | TAGAATACTGGCATCTGTTTTT |
| 130 | E45SaCas9KKH5 | TGGCATCTGTTTTTGAGGATTG |
| 131 | E45SaCas9KKH6 | AGGATTGCTGAATTATTTCTTC |
| 132 | E45SaCas9KKH7 | AATTATTTCTTCCCCAGTTGCA |
| 133 | E45SaCas9KKH8 | CAGTTGCATTCAATGTTCTGAC |
| 134 | E45SaCas9KKH9 | TTCTGACAACAGTTTGCCGCTG |
| 135 | E45SaCas9KKH10 | TTGCCGCTGCCCAATGCCATCC |
| 136 | E45SaCas9KKH16 | AAACAGCTGTCAGACAGAAAAA |
| 137 | E45SaCas9KKH17 | GAAGCCTGAATCTGCGGTGGCA |
| 138 | E45SaCas9KKH18 | GAAAAATTGGGAAGCCTGAATC |
| 139 | E45SaCas9KKH19 | TCTACAGGAAAAATTGGGAAGC |
| 140 | E45SaCas9KKH20 | ACAGATGCCAGTATTCTACAGG |
| 141 | E45SaCas9KKH21 | TCAGCAATCCTCAAAAACAGAT |
| 142 | E45SaCas9KKH22 | AATAATTCAGCAATCCTCAAAA |
| 143 | E45SaCas9KKH23 | GCAACTGGGGAAGAAATAATTC |
| 144 | E45SaCas9KKH24 | CATTGAATGCAACTGGGGAAGA |
| 145 | E45SaCas9KKH25 | GAACATTGAATGCAACTGGGGA |
| 146 | E45SaCas9KKH26 | GCGGCAAACTGTTGTCAGAACA |
| 147 | E45SaCas9KKH27 | TTTTGGTATCTTACAGGAACTC |

TABLE 2B
(2) Exon 45 SaCas9 pairs:

| Guide 1 Seq ID | | | Guide 2 Seq ID | | |
|---|---|---|---|---|---|
| No. | Guide | Guide 1 Sequence | No. | Guide | Guide 2 Sequence |
| 3 | E45Sa1 | TCAGGCTTCCCAATTTTTC | 4 | E45Sa2 | TAGAATACTGGCATCTGTT |
| | | CTG | | | TTT |
| 3 | E45Sa1 | TCAGGCTTCCCAATTTTTC | 5 | E45Sa3 | TGGCATCTGTTTTTGAGGA |
| | | CTG | | | TTG |
| 3 | E45Sa1 | TCAGGCTTCCCAATTTTTC | 6 | E45Sa4 | TTGCCGCTGCCCAATGCCA |
| | | CTG | | | TCC |
| 3 | E45Sa1 | TCAGGCTTCCCAATTTTTC | 7 | E45Sa6 | TCTACAGGAAAAATTGGGA |
| | | CTG | | | AGC |
| 3 | E45Sa1 | TCAGGCTTCCCAATTTTTC | 8 | E45Sa7 | GCGGCAAACTGTTGTCAGA |
| | | CTG | | | ACA |
| 3 | E45Sa1 | TCAGGCTTCCCAATTTTTC | 9 | E45Sa8 | TTTTGGTATCTTACAGGAA |
| | | CTG | | | CTC |
| 4 | E45Sa2 | TAGAATACTGGCATCTGTT | 5 | E45Sa3 | TGGCATCTGTTTTTGAGGA |
| | | TTT | | | TTG |
| 4 | E45Sa2 | TAGAATACTGGCATCTGTT | 6 | E45Sa4 | TTGCCGCTGCCCAATGCCA |
| | | TTT | | | TCC |
| 4 | E45Sa2 | TAGAATACTGGCATCTGTT | 7 | E45Sa6 | TCTACAGGAAAAATTGGGA |
| | | TTT | | | AGC |
| 4 | E45Sa2 | TAGAATACTGGCATCTGTT | 8 | E45Sa7 | GCGGCAAACTGTTGTCAGA |
| | | TTT | | | ACA |
| 4 | E45Sa2 | TAGAATACTGGCATCTGTT | 9 | E45Sa8 | TTTTGGTATCTTACAGGAA |
| | | TTT | | | CTC |
| 5 | E45Sa3 | TGGCATCTGTTTTTGAGGA | 6 | E45Sa4 | TTGCCGCTGCCCAATGCCA |
| | | TTG | | | TCC |
| 5 | E45Sa3 | TGGCATCTGTTTTTGAGGA | 7 | E45Sa6 | TCTACAGGAAAAATTGGGA |
| | | TTG | | | AGC |
| 5 | E45Sa3 | TGGCATCTGTTTTTGAGGA | 8 | E45Sa7 | GCGGCAAACTGTTGTCAGA |
| | | TTG | | | ACA |
| 5 | E45Sa3 | TGGCATCTGTTTTTGAGGA | 9 | E45Sa8 | TTTTGGTATCTTACAGGAA |
| | | TTG | | | CTC |
| 6 | E45Sa4 | TTGCCGCTGCCCAATGCCA | 7 | E45Sa6 | TCTACAGGAAAAATTGGGA |
| | | TCC | | | AGC |
| 6 | E45Sa4 | TTGCCGCTGCCCAATGCCA | 8 | E45Sa7 | GCGGCAAACTGTTGTCAGA |
| | | TCC | | | ACA |
| 6 | E45Sa4 | TTGCCGCTGCCCAATGCCA | 9 | E45Sa8 | TTTTGGTATCTTACAGGAA |
| | | TCC | | | CTC |
| 7 | E45Sa6 | TCTACAGGAAAAATTGGGA | 8 | E45Sa7 | GCGGCAAACTGTTGTCAGA |
| | | AGC | | | ACA |
| 7 | E45Sa6 | TCTACAGGAAAAATTGGGA | 9 | E45Sa8 | TTTTGGTATCTTACAGGAA |
| | | AGC | | | CTC |
| 8 | E45Sa7 | GCGGCAAACTGTTGTCAGA | 9 | E45Sa8 | TTTTGGTATCTTACAGGAA |
| | | ACA | | | CTC |
| 126 | E45SaCas9KKH1 | TGTTTGCAGACCTCCTGCC | 127 | E45SaCas9KKH2 | CTGCCACCGCAGATTCAGG |
| | | ACC | | | CTT |
| 126 | E45SaCas9KKH1 | TGTTTGCAGACCTCCTGCC | 128 | E45SaCas9KKH3 | TCAGGCTTCCCAATTTTTC |
| | | ACC | | | CTG |
| 126 | E45SaCas9KKH1 | TGTTTGCAGACCTCCTGCC | 129 | E45SaCas9KKH4 | TAGAATACTGGCATCTGTT |
| | | ACC | | | TTT |
| 126 | E45SaCas9KKH1 | TGTTTGCAGACCTCCTGCC | 130 | E45SaCas9KKH8 | TGGCATCTGTTTTTGAGGA |
| | | ACC | | | TTG |
| 126 | E45SaCas9KKH1 | TGTTTGCAGACCTCCTGCC | 131 | E45SaCas9KKH6 | AGGATTGCTGAATTATTTC |
| | | ACC | | | TTC |
| 126 | E45SaCas9KKH1 | TGTTTGCAGACCTCCTGCC | 132 | E45SaCas9KKH7 | AATTATTTCTTCCCCAGTT |
| | | ACC | | | GCA |
| 126 | E45SaCas9KKH1 | TGTTTGCAGACCTCCTGCC | 133 | E45SaCas9KKH8 | CAGTTGCATTCAATGTTCT |
| | | ACC | | | GAC |
| 126 | E45SaCas9KKH1 | TGTTTGCAGACCTCCTGCC | 134 | E45SaCas9KKH9 | TTCTGACAACAGTTTGCCG |
| | | ACC | | | CTG |
| 126 | E45SaCas9KKH1 | TGTTTGCAGACCTCCTGCC | 135 | E45SaCas9KKH10 | TTGCCGCTGCCCAATGCCA |
| | | ACC | | | TCC |
| 126 | E45SaCas9KKH1 | TGTTTGCAGACCTCCTGCC | 136 | E45SaCas9KKH16 | AAACAGCTGTCAGACAGAA |
| | | ACC | | | AAA |
| 126 | E45SaCas9KKH1 | TGTTTGCAGACCTCCTGCC | 137 | E45SaCas9KKH17 | GAAGCCTGAATCTGCGGTG |
| | | ACC | | | GCA |
| 126 | E45SaCas9KKH1 | TGTTTGCAGACCTCCTGCC | 138 | E45SaCas9KKH18 | GAAAAATTGGGAAGCCTGA |
| | | ACC | | | ATC |
| 126 | E45SaCas9KKH1 | TGTTTGCAGACCTCCTGCC | 139 | E45SaCas9KKH19 | TCTACAGGAAAAATTGGGA |
| | | ACC | | | AGC |
| 126 | E45SaCas9KKH1 | TGTTTGCAGACCTCCTGCC | 140 | E45SaCas9KKH20 | ACAGATGCCAGTATTCTAC |
| | | ACC | | | AGG |
| 126 | E45SaCas9KKH1 | TGTTTGCAGACCTCCTGCC | 141 | E45SaCas9KKH21 | TCAGCAATCCTCAAAAACA |
| | | ACC | | | GAT |
| 126 | E45SaCas9KKH1 | TGTTTGCAGACCTCCTGCC | 142 | E45SaCas9KKH22 | AATAATTCAGCAATCCTCA |
| | | ACC | | | AAA |
| 126 | E45SaCas9KKH1 | TGTTTGCAGACCTCCTGCC | 143 | E45SaCas9KKH23 | GCAACTGGGGAAGAAATAA |
| | | ACC | | | TTC |
| 126 | E45SaCas9KKH1 | TGTTTGCAGACCTCCTGCC | 144 | E45SaCas9KKH24 | CATTGAATGCAACTGGGGA |
| | | ACC | | | AGA |
| 126 | E45SaCas9KKH1 | TGTTTGCAGACCTCCTGCC | 145 | E45SaCas9KKH25 | GAACATTGAATGCAACTGG |
| | | ACC | | | GGA |
| 126 | E45SaCas9KKH1 | TGTTTGCAGACCTCCTGCC | 146 | E45SaCas9KKH26 | GCGGCAAACTGTTGTCAGA |
| | | ACC | | | ACA |
| 126 | E45SaCas9KKH1 | TGTTTGCAGACCTCCTGCC | 147 | E45SaCas9KKH27 | TTTTGGTATCTTACAGGAA |
| | | ACC | | | CTC |
| 127 | E45SaCas9KKH2 | CTGCCACCGCAGATTCAGG | 128 | E45SaCas9KKH3 | TCAGGCTTCCCAATTTTTC |
| | | CTT | | | CTG |
| 127 | E45SaCas9KKH2 | CTGCCACCGCAGATTCAGG | 129 | E45SaCas9KKH4 | TAGAATACTGGCATCTGTT |
| | | CTT | | | TTT |
| 127 | E45SaCas9KKH2 | CTGCCACCGCAGATTCAGG | 130 | E45SaCas9KKH5 | TGGCATCTGTTTTTGAGGA |
| | | CTT | | | TTG |
| 127 | E45SaCas9KKH2 | CTGCCACCGCAGATTCAGG | 131 | E45SaCas9KKH6 | AGGATTGCTGAATTATTTC |
| | | CTT | | | TTC |
| 127 | E45SaCas9KKH2 | CTGCCACCGCAGATTCAGG | 132 | E45SaCas9KKH7 | AATTATTTCTTCCCCAGTT |
| | | CTT | | | GCA |
| 127 | E45SaCas9KKH2 | CTGCCACCGCAGATTCAGG | 133 | E45SaCas9KKH8 | CAGTTGCATTCAATGTTCT |
| | | CTT | | | GAC |
| 127 | E45SaCas9KKH2 | CTGCCACCGCAGATTCAGG | 134 | E45SaCas9KKH9 | TTCTGACAACAGTTTGCCG |
| | | CTT | | | CTG |
| 127 | E45SaCas9KKH2 | CTGCCACCGCAGATTCAGG | 135 | E45SaCas9KKH10 | TTGCCGCTGCCCAATGCCA |
| | | CTT | | | TCC |
| 127 | E45SaCas9KKH2 | CTGCCACCGCAGATTCAGG | 136 | E45SaCas9KKH16 | AAACAGCTGTCAGACAGAA |
| | | CTT | | | AAA |
| 127 | E45SaCas9KKH2 | CTGCCACCGCAGATTCAGG | 137 | E45SaCas9KKH17 | GAAGCCTGAATCTGCGGTG |
| | | CTT | | | GCA |
| 127 | E45SaCas9KKH2 | CTGCCACCGCAGATTCAGG | 138 | E45SaCas9KKH18 | GAAAAATTGGGAAGCCTGA |
| | | CTT | | | ATC |
| 127 | E45SaCas9KKH2 | CTGCCACCGCAGATTCAGG | 139 | E45SaCas9KKH19 | TCTACAGGAAAAATTGGGA |
| | | CTT | | | AGC |
| 127 | E45SaCas9KKH2 | CTGCCACCGCAGATTCAGG | 140 | E45SaCas9KKH20 | ACAGATGCCAGTATTCTAC |
| | | CTT | | | AGG |
| 127 | E45SaCas9KKH2 | CTGCCACCGCAGATTCAGG | 141 | E45SaCas9KKH21 | TCAGCAATCCTCAAAAACA |
| | | CTT | | | GAT |
| 127 | E45SaCas9KKH2 | CTGCCACCGCAGATTCAGG | 142 | E45SaCas9KKH22 | AATAATTCAGCAATCCTCA |
| | | CTT | | | AAA |
| 127 | E45SaCas9KKH2 | CTGCCACCGCAGATTCAGG | 143 | E45SaCas9KKH23 | GCAACTGGGGAAGAAATAA |
| | | CTT | | | TTC |
| 127 | E45SaCas9KKH2 | CTGCCACCGCAGATTCAGG | 144 | E45SaCas9KKH24 | CATTGAATGCAACTGGGGA |
| | | CTT | | | AGA |
| 127 | E45SaCas9KKH2 | CTGCCACCGCAGATTCAGG | 145 | E45SaCas9KKH25 | GAACATTGAATGCAACTGG |
| | | CTT | | | GGA |
| 127 | E45SaCas9KKH2 | CTGCCACCGCAGATTCAGG | 146 | E45SaCas9KKH26 | GCGGCAAACTGTTGTCAGA |
| | | CTT | | | ACA |
| 127 | E45SaCas9KKH2 | CTGCCACCGCAGATTCAGG | 147 | E45SaCas9KKH27 | TTTTGGTATCTTACAGGAA |
| | | CTT | | | CTC |
| 128 | E45SaCas9KKH3 | TCAGGCTTCCCAATTTTTC | 129 | E45SaCas9KKH4 | TAGAATACTGGCATCTGTT |
| | | CTG | | | TTT |
| 128 | E45SaCas9KKH3 | TCAGGCTTCCCAATTTTTC | 130 | E45SaCas9KKH5 | TGGCATCTGTTTTTGAGGA |
| | | CTG | | | TTG |
| 128 | E45SaCas9KKH3 | TCAGGCTTCCCAATTTTTC | 131 | E45SaCas9KKH6 | AGGATTGCTGAATTATTTC |
| | | CTG | | | TTC |
| 128 | E45SaCas9KKH3 | TCAGGCTTCCCAATTTTTC | 132 | E45SaCas9KKH7 | AATTATTTCTTCCCCAGTT |
| | | CTG | | | GCA |
| 128 | E45SaCas9KKH3 | TCAGGCTTCCCAATTTTTC | 133 | E45SaCas9KKH8 | CAGTTGCATTCAATGTTCT |
| | | CTG | | | GAC |
| 128 | E45SaCas9KKH3 | TCAGGCTTCCCAATTTTTC | 134 | E45SaCas9KKH9 | TTCTGACAACAGTTTGCCG |
| | | CTG | | | CTG |
| 128 | E45SaCas9KKH3 | TCAGGCTTCCCAATTTTTC | 135 | E45SaCas9KKH10 | TTGCCGCTGCCCAATGCCA |
| | | CTG | | | TCC |
| 128 | E45SaCas9KKH3 | TCAGGCTTCCCAATTTTTC | 136 | E45SaCas9KKH16 | AAACAGCTGTCAGACAGAA |
| | | CTG | | | AAA |
| 128 | E45SaCas9KKH3 | TCAGGCTTCCCAATTTTTC | 137 | E45SaCas9KKH17 | GAAGCCTGAATCTGCGGTG |
| | | CTG | | | GCA |
| 128 | E45SaCas9KKH3 | TCAGGCTTCCCAATTTTTC | 138 | E45SaCas9KKH18 | GAAAAATTGGGAAGCCTGA |
| | | CTG | | | ATC |
| 128 | E45SaCas9KKH3 | TCAGGCTTCCCAATTTTTC | 139 | E45SaCas9KKH19 | TCTACAGGAAAAATTGGGA |
| | | CTG | | | AGC |
| 128 | E45SaCas9KKH3 | TCAGGCTTCCCAATTTTTC | 140 | E45SaCas9KKH20 | ACAGATGCCAGTATTCTAC |
| | | CTG | | | AGG |
| 128 | E45SaCas9KKH3 | TCAGGCTTCCCAATTTTTC | 141 | E45SaCas9KKH21 | TCAGCAATCCTCAAAAACA |
| | | CTG | | | GAT |
| 128 | E45SaCas9KKH3 | TCAGGCTTCCCAATTTTTC | 142 | E45SaCas9KKH22 | AATAATTCAGCAATCCTCA |
| | | CTG | | | AAA |
| 128 | E45SaCas9KKH3 | TCAGGCTTCCCAATTTTTC | 143 | E45SaCas9KKH23 | GCAACTGGGGAAGAAATAA |
| | | CTG | | | TTC |
| 128 | E45SaCas9KKH3 | TCAGGCTTCCCAATTTTTC | 144 | E45SaCas9KKH24 | CATTGAATGCAACTGGGGA |
| | | CTG | | | AGA |
| 128 | E45SaCas9KKH3 | TCAGGCTTCCCAATTTTTC | 145 | E45SaCas9KKH25 | GAACATTGAATGCAACTGG |
| | | CTG | | | GGA |
| 128 | E45SaCas9KKH3 | TCAGGCTTCCCAATTTTTC | 146 | E45SaCas9KKH26 | GCGGCAAACTGTTGTCAGA |
| | | CTG | | | ACA |
| 128 | E45SaCas9KKH3 | TCAGGCTTCCCAATTTTTC | 147 | E45SaCas9KKH27 | TTTTGGTATCTTACAGGAA |
| | | CTG | | | CTC |
| 129 | E45SaCas9KKH4 | TAGAATACTGGCATCTGTT | 130 | E45SaCas9KKH5 | TGGCATCTGTTTTTGAGGA |
| | | TTT | | | TTG |
| 129 | E45SaCas9KKH4 | TAGAATACTGGCATCTGTT | 131 | E45SaCas9KKH6 | AGGATTGCTGAATTATTTC |
| | | TTT | | | TTC |
| 129 | E45SaCas9KKH4 | TAGAATACTGGCATCTGTT | 132 | E45SaCas9KKH7 | AATTATTTCTTCCCCAGTT |
| | | TTT | | | GCA |
| 129 | E45SaCas9KKH4 | TAGAATACTGGCATCTGTT | 133 | E45SaCas9KKH8 | CAGTTGCATTCAATGTTCT |
| | | TTT | | | GAC |
| 129 | E45SaCas9KKH4 | TAGAATACTGGCATCTGTT | 134 | E45SaCas9KKH9 | TTCTGACAACAGTTTGCCG |
| | | TTT | | | CTG |
| 129 | E45SaCas9KKH4 | TAGAATACTGGCATCTGTT | 135 | E45SaCas9KKH10 | TTGCCGCTGCCCAATGCCA |
| | | TTT | | | TCC |
| 129 | E45SaCas9KKH4 | TAGAATACTGGCATCTGTT | 136 | E45SaCas9KKH16 | AAACAGCTGTCAGACAGAA |
| | | TTT | | | AAA |
| 129 | E45SaCas9KKH4 | TAGAATACTGGCATCTGTT | 137 | E45SaCas9KKH17 | GAAGCCTGAATCTGCGGTG |
| | | TTT | | | GCA |
| 129 | E45SaCas9KKH4 | TAGAATACTGGCATCTGTT | 138 | E45SaCas9KKH18 | GAAAAATTGGGAAGCCTGA |
| | | TTT | | | ATC |
| 129 | E45SaCas9KKH4 | TAGAATACTGGCATCTGTT | 139 | E45SaCas9KKH19 | TCTACAGGAAAAATTGGGA |
| | | TTT | | | AGC |
| 129 | E45SaCas9KKH4 | TAGAATACTGGCATCTGTT | 140 | E45SaCas9KKH20 | ACAGATGCCAGTATTCTAC |
| | | TTT | | | AGG |
| 129 | E45SaCas9KKH4 | TAGAATACTGGCATCTGTT | 141 | E45SaCas9KKH21 | TCAGCAATCCTCAAAAACA |
| | | TTT | | | GAT |
| 129 | E45SaCas9KKH4 | TAGAATACTGGCATCTGTT | 142 | E45SaCas9KKH2 | AATAATTCAGCAATCCTCA |
| | | TTT | | | AAA |
| 129 | E45SaCas9KKH4 | TAGAATACTGGCATCTGTT | 143 | E45SaCas9KKH23 | GCAACTGGGGAAGAAATAA |
| | | TTT | | | TTC |
| 129 | E45SaCas9KKH4 | TAGAATACTGGCATCTGTT | 144 | E45SaCas9KKH24 | CATTGAATGCAACTGGGGA |
| | | TTT | | | AGA |
| 129 | E45SaCas9KKH4 | TAGAATACTGGCATCTGTT | 145 | E45SaCas9KKH25 | GAACATTGAATGCAACTGG |
| | | TTT | | | GGA |
| 129 | E45SaCas9KKH4 | TAGAATACTGGCATCTGTT | 146 | E45SaCas9KKH26 | GCGGCAAACTGTTGTCAGA |
| | | TTT | | | ACA |
| 129 | E45SaCas9KKH4 | TAGAATACTGGCATCTGTT | 147 | E45SaCas9KKH27 | TTTTGGTATCTTACAGGAA |
| | | TTT | | | CTC |
| 130 | E45SaCas9KKH5 | TGGCATCTGTTTTTGAGGA | 131 | E45SaCas9KKH6 | AGGATTGCTGAATTATTTC |
| | | TTG | | | TTC |
| 130 | E45SaCas9KKH5 | TGGCATCTGTTTTTGAGGA | 132 | E45SaCas9KKH7 | AATTATTTCTTCCCCAGTT |
| | | TTG | | | GCA |
| 130 | E45SaCas9KKH5 | TGGCATCTGTTTTTGAGGA | 133 | E45SaCas9KKH8 | CAGTTGCATTCAATGTTCT |
| | | TTG | | | GAC |
| 130 | E45SaCas9KKH5 | TGGCATCTGTTTTTGAGGA | 134 | E45SaCas9KKH9 | TTCTGACAACAGTTTGCCG |
| | | TTG | | | CTG |
| 130 | E45SaCas9KKH5 | TGGCATCTGTTTTTGAGGA | 135 | E45SaCas9KKH10 | TTGCCGCTGCCCAATGCCA |
| | | TTG | | | TCC |
| 130 | E45SaCas9KKH5 | TGGCATCTGTTTTTGAGGA | 136 | E45SaCas9KKH16 | AAACAGCTGTCAGACAGAA |
| | | TTG | | | AAA |
| 130 | E45SaCas9KKH5 | TGGCATCTGTTTTTGAGGA | 137 | E45SaCas9KKH17 | GAAGCCTGAATCTGCGGTG |
| | | TTG | | | GCA |
| 130 | E45SaCas9KKH5 | TGGCATCTGTTTTTGAGGA | 138 | E45SaCas9KKH18 | GAAAAATTGGGAAGCCTGA |
| | | TTG | | | ATC |
| 130 | E45SaCas9KKH5 | TGGCATCTGTTTTTGAGGA | 139 | E45SaCas9KKH19 | TCTACAGGAAAAATTGGGA |
| | | TTG | | | AGC |
| 130 | E45SaCas9KKH5 | TGGCATCTGTTTTTGAGGA | 140 | E45SaCas9KKH20 | ACAGATGCCAGTATTCTAC |
| | | TTG | | | AGG |
| 130 | E45SaCas9KKH5 | TGGCATCTGTTTTTGAGGA | 141 | E45SaCas9KKH21 | TCAGCAATCCTCAAAAACA |
| | | TTG | | | GAT |
| 130 | E45SaCas9KKH5 | TGGCATCTGTTTTTGAGGA | 142 | E45SaCas9KKH22 | AATAATTCAGCAATCCTCA |
| | | TTG | | | AAA |
| 130 | E45SaCas9KKH5 | TGGCATCTGTTTTTGAGGA | 143 | E45SaCas9KKH23 | GCAACTGGGGAAGAAATAA |
| | | TTG | | | TTC |
| 130 | E45SaCas9KKH5 | TGGCATCTGTTTTTGAGGA | 144 | E45SaCas9KKH24 | CATTGAATGCAACTGGGGA |
| | | TTG | | | AGA |
| 130 | E45SaCas9KKH5 | TGGCATCTGTTTTTGAGGA | 145 | E45SaCas9KKH25 | GAACATTGAATGCAACTGG |
| | | TTG | | | GGA |
| 130 | E45SaCas9KKH5 | TGGCATCTGTTTTTGAGGA | 146 | E45SaCas9KKH26 | GCGGCAAACTGTTGTCAGA |
| | | TTG | | | ACA |
| 130 | E45SaCas9KKH5 | TGGCATCTGTTTTTGAGGA | 147 | E45SaCas9KKH27 | TTTTGGTATCTTACAGGAA |
| | | TTG | | | CTC |
| 131 | E45SaCas9KKH6 | AGGATTGCTGAATTATTTC | 132 | E45SaCas9KKH7 | AATTATTTCTTCCCCAGTT |
| | | TTC | | | GCA |
| 131 | E45SaCas9KKH6 | AGGATTGCTGAATTATTTC | 133 | E45SaCas9KKH8 | CAGTTGCATTCAATGTTCT |
| | | TTC | | | GAC |
| 131 | E45SaCas9KKH6 | AGGATTGCTGAATTATTTC | 134 | E45SaCas9KKH9 | TTCTGACAACAGTTTGCCG |
| | | TTC | | | CTG |
| 131 | E45SaCas9KKH6 | AGGATTGCTGAATTATTTC | 135 | E45SaCas9KKH10 | TTGCCGCTGCCCAATGCCA |
| | | TTC | | | TCC |
| 131 | E45SaCas9KKH6 | AGGATTGCTGAATTATTTC | 136 | E45SaCas9KKH16 | AAACAGCTGTCAGACAGAA |
| | | TTC | | | AAA |
| 131 | E45SaCas9KKH6 | AGGATTGCTGAATTATTTC | 137 | E45SaCas9KKH17 | GAAGCCTGAATCTGCGGTG |
| | | TTC | | | GCA |
| 131 | E45SaCas9KKH6 | AGGATTGCTGAATTATTTC | 138 | E45SaCas9KKH18 | GAAAAATTGGGAAGCCTGA |
| | | TTC | | | ATC |
| 131 | E45SaCas9KKH6 | AGGATTGCTGAATTATTTC | 139 | E45SaCas9KKH19 | TCTACAGGAAAAATTGGGA |
| | | TTC | | | AGC |
| 131 | E45SaCas9KKH6 | AGGATTGCTGAATTATTTC | 140 | E45SaCas9KKH20 | ACAGATGCCAGTATTCTAC |
| | | TTC | | | AGG |
| 131 | E45SaCas9KKH6 | AGGATTGCTGAATTATTTC | 141 | E45SaCas9KKH21 | TCAGCAATCCTCAAAAACA |
| | | TTC | | | GAT |
| 131 | E45SaCas9KKH6 | AGGATTGCTGAATTATTTC | 142 | E45SaCas9KKH22 | AATAATTCAGCAATCCTCA |
| | | TTC | | | AAA |
| 131 | E45SaCas9KKH6 | AGGATTGCTGAATTATTTC | 143 | E45SaCas9KKH23 | GCAACTGGGGAAGAAATAA |
| | | TTC | | | TTC |
| 131 | E45SaCas9KKH6 | AGGATTGCTGAATTATTTC | 144 | E45SaCas9KKH24 | CATTGAATGCAACTGGGGA |
| | | TTC | | | AGA |
| 131 | E45SaCas9KKH6 | AGGATTGCTGAATTATTTC | 145 | E45SaCas9KKH25 | GAACATTGAATGCAACTGG |
| | | TTC | | | GGA |
| 131 | E45SaCas9KKH6 | AGGATTGCTGAATTATTTC | 146 | E45SaCas9KKH26 | GCGGCAAACTGTTGTCAGA |
| | | TTC | | | ACA |
| 131 | E45SaCas9KKH6 | AGGATTGCTGAATTATTTC | 147 | E45SaCas9KKH27 | TTTTGGTATCTTACAGGAA |
| | | TTC | | | CTC |
| 132 | E45SaCas9KKH7 | AATTATTTCTTCCCCAGTT | 133 | E45SaCas9KKH8 | CAGTTGCATTCAATGTTCT |
| | | GCA | | | GAC |
| 132 | E45SaCas9KKH7 | AATTATTTCTTCCCCAGTT | 134 | E45SaCas9KKH9 | TTCTGACAACAGTTTGCCG |
| | | GCA | | | CTG |
| 132 | E45SaCas9KKH7 | AATTATTTCTTCCCCAGTT | 135 | E45SaCas9KKH10 | TTGCCGCTGCCCAATGCCA |
| | | GCA | | | TCC |
| 132 | E45SaCas9KKH7 | AATTATTTCTTCCCCAGTT | 136 | E45SaCas9KKH16 | AAACAGCTGTCAGACAGAA |
| | | GCA | | | AAA |
| 132 | E45SaCas9KKH7 | AATTATTTCTTCCCCAGTT | 137 | E45SaCas9KKH17 | GAAGCCTGAATCTGCGGTG |
| | | GCA | | | GCA |
| 132 | E45SaCas9KKH7 | AATTATTTCTTCCCCAGTT | 138 | E45SaCas9KKH18 | GAAAAATTGGGAAGCCTGA |
| | | GCA | | | ATC |
| 132 | E45SaCas9KKH7 | AATTATTTCTTCCCCAGTT | 139 | E45SaCas9KKH19 | TCTACAGGAAAAATTGGGA |
| | | GCA | | | AGC |
| 132 | E45SaCas9KKH7 | AATTATTTCTTCCCCAGTT | 140 | E45SaCas9KKH20 | ACAGATGCCAGTATTCTAC |
| | | GCA | | | AGG |
| 132 | E45SaCas9KKH7 | AATTATTTCTTCCCCAGTT | 141 | E45SaCas9KKH21 | TCAGCAATCCTCAAAAACA |
| | | GCA | | | GAT |
| 132 | E45SaCas9KKH7 | AATTATTTCTTCCCCAGTT | 142 | E45SaCas9KKH22 | AATAATTCAGCAATCCTCA |
| | | GCA | | | AAA |
| 132 | E45SaCas9KKH7 | AATTATTTCTTCCCCAGTT | 143 | E45SaCas9KKH23 | GCAACTGGGGAAGAAATAA |
| | | GCA | | | TTC |
| 132 | E45SaCas9KKH7 | AATTATTTCTTCCCCAGTT | 144 | E45SaCas9KKH24 | CATTGAATGCAACTGGGGA |
| | | GCA | | | AGA |
| 132 | E45SaCas9KKH7 | AATTATTTCTTCCCCAGTT | 145 | E45SaCas9KKH25 | GAACATTGAATGCAACTGG |
| | | GCA | | | GGA |
| 132 | E45SaCas9KKH7 | AATTATTTCTTCCCCAGTT | 146 | E45SaCas9KKH26 | GCGGCAAACTGTTGTCAGA |
| | | GCA | | | ACA |
| 132 | E45SaCas9KKH7 | AATTATTTCTTCCCCAGTT | 147 | E45SaCas9KKH27 | TTTTGGTATCTTACAGGAA |
| | | GCA | | | CTC |
| 133 | E45SaCas9KKH8 | CAGTTGCATTCAATGTTCT | 134 | E45SaCas9KKH9 | TTCTGACAACAGTTTGCCG |
| | | GAC | | | CTG |
| 133 | E45SaCas9KKH8 | CAGTTGCATTCAATGTTCT | 135 | E45SaCas9KKH10 | TTGCCGCTGCCCAATGCCA |
| | | GAC | | | TCC |
| 133 | E45SaCas9KKH8 | CAGTTGCATTCAATGTTCT | 136 | E45SaCas9KKH16 | AAACAGCTGTCAGACAGAA |
| | | GAC | | | AAA |
| 133 | E45SaCas9KKH8 | CAGTTGCATTCAATGTTCT | 137 | E45SaCas9KKH17 | GAAGCCTGAATCTGCGGTG |
| | | GAC | | | GCA |
| 133 | E45SaCas9KKH8 | CAGTTGCATTCAATGTTCT | 138 | E45SaCas9KKH18 | GAAAAATTGGGAAGCCTGA |
| | | GAC | | | ATC |
| 133 | E45SaCas9KKH8 | CAGTTGCATTCAATGTTCT | 139 | E45SaCas9KKH19 | TCTACAGGAAAAATTGGGA |
| | | GAC | | | AGC |
| 133 | E45SaCas9KKH8 | CAGTTGCATTCAATGTTCT | 140 | E45SaCas9KKH20 | ACAGATGCCAGTATTCTAC |
| | | GAC | | | AGG |
| 133 | E45SaCas9KKH8 | CAGTTGCATTCAATGTTCT | 141 | E45SaCas9KKH21 | TCAGCAATCCTCAAAAACA |
| | | GAC | | | GAT |
| 133 | E45SaCas9KKH8 | CAGTTGCATTCAATGTTCT | 142 | E45SaCas9KKH22 | AATAATTCAGCAATCCTCA |
| | | GAC | | | AAA |
| 133 | E45SaCas9KKH8 | CAGTTGCATTCAATGTTCT | 143 | E45SaCas9KKH23 | GCAACTGGGGAAGAAATAA |
| | | GAC | | | TTC |
| 133 | E45SaCas9KKH8 | CAGTTGCATTCAATGTTCT | 144 | E45SaCas9KKH24 | CATTGAATGCAACTGGGGA |
| | | GAC | | | AGA |
| 133 | E45SaCas9KKH8 | CAGTTGCATTCAATGTTCT | 145 | E45SaCas9KKH25 | GAACATTGAATGCAACTGG |
| | | GAC | | | GGA |
| 133 | E45SaCas9KKH8 | CAGTTGCATTCAATGTTCT | 146 | E45SaCas9KKH26 | GCGGCAAACTGTTGTCAGA |
| | | GAC | | | ACA |
| 133 | E45SaCas9KKH8 | CAGTTGCATTCAATGTTCT | 147 | E45SaCas9KKH27 | TTTTGGTATCTTACAGGAA |
| | | GAC | | | CTC |
| 134 | E45SaCas9KKH9 | TTCTGACAACAGTTTGCCG | 135 | E45SaCas9KKH10 | TTGCCGCTGCCCAATGCCA |
| | | CTG | | | TCC |
| 134 | E45SaCas9KKH9 | TTCTGACAACAGTTTGCCG | 136 | E45SaCas9KKH16 | AAACAGCTGTCAGACAGAA |
| | | CTG | | | AAA |
| 134 | E45SaCas9KKH9 | TTCTGACAACAGTTTGCCG | 137 | E45SaCas9KKH17 | GAAGCCTGAATCTGCGGTG |
| | | CTG | | | GCA |
| 134 | E45SaCas9KKH9 | TTCTGACAACAGTTTGCCG | 138 | E45SaCas9KKH18 | GAAAAATTGGGAAGCCTGA |
| | | CTG | | | ATC |
| 134 | E45SaCas9KKH9 | TTCTGACAACAGTTTGCCG | 139 | E45SaCas9KKH19 | TCTACAGGAAAAATTGGGA |
| | | CTG | | | AGC |
| 134 | E45SaCas9KKH9 | TTCTGACAACAGTTTGCCG | 140 | E45SaCas9KKH20 | ACAGATGCCAGTATTCTAC |
| | | CTG | | | AGG |
| 134 | E45SaCas9KKH9 | TTCTGACAACAGTTTGCCG | 141 | E45SaCas9KKH21 | TCAGCAATCCTCAAAAACA |
| | | CTG | | | GAT |
| 134 | E45SaCas9KKH9 | TTCTGACAACAGTTTGCCG | 142 | E45SaCas9KKH22 | AATAATTCAGCAATC TCA |
| | | CTG | | | AAA |
| 134 | E45SaCas9KKH9 | TTCTGACAACAGTTTGCCG | 143 | E45SaCas9KKH23 | GCAACTGGGGAAGAAATAA |
| | | CTG | | | TTC |
| 134 | E45SaCas9KKH9 | TTCTGACAACAGTTTGCCG | 144 | E45SaCas9KKH24 | CATTGAATGCAACTGGGGA |
| | | CTG | | | AGA |
| 134 | E45SaCas9KKH9 | TTCTGACAACAGTTTGCCG | 145 | E45SaCas9KKH25 | GAACATTGAATGCAACTGG |
| | | CTG | | | GGA |
| 134 | E45SaCas9KKH9 | TTCTGACAACAGTTTGCCG | 146 | E45SaCas9KKH26 | GCGGCAAACTGTTGTCAGA |
| | | CTG | | | ACA |
| 134 | E45SaCas9KKH9 | TTCTGACAACAGTTTGCCG | 147 | E45SaCas9KKH27 | TTTTGGTATCTTACAGGAA |
| | | CTG | | | CTC |
| 135 | E45SaCas9KKH10 | TTGCCGCTGCCCAATGCCA | 136 | E45SaCas9KKH16 | AAACAGCTGTCAGACAGAA |
| | | TCC | | | AAA |
| 135 | E45SaCas9KKH10 | TTGCCGCTGCCCAATGCCA | 137 | E45SaCas9KKH17 | GAAGCCTGAATCTGCGGTG |
| | | TCC | | | GCA |
| 135 | E45SaCas9KKH10 | TTGCCGCTGCCCAATGCCA | 138 | E45SaCas9KKH18 | GAAAAATTGGGAAGCCTGA |
| | | TCC | | | ATC |
| 135 | E45SaCas9KKH10 | TTGCCGCTGCCCAATGCCA | 139 | E45SaCas9KKH19 | TCTACAGGAAAAATTGGGA |
| | | TCC | | | AGC |
| 135 | E45SaCas9KKH10 | TTGCCGCTGCCCAATGCCA | 140 | E45SaCas9KKH20 | ACAGATGCCAGTATTCTAC |
| | | TCC | | | AGG |
| 135 | E45SaCas9KKH10 | TTGCCGCTGCCCAATGCCA | 141 | E45SaCas9KKH21 | TCAGCAATCCTCAAAAACA |
| | | TCC | | | GAT |
| 135 | E45SaCas9KKH10 | TTGCCGCTGCCCAATGCCA | 142 | E45SaCas9KKH22 | AATAATTCAGCAATCCTCA |
| | | TCC | | | AAA |
| 135 | E45SaCas9KKH10 | TTGCCGCTGCCCAATGCCA | 143 | E45SaCas9KKH23 | GCAACTGGGGAAGAAATAA |
| | | TCC | | | TTC |
| 135 | E45SaCas9KKH10 | TTGCCGCTGCCCAATGCCA | 144 | E45SaCas9KKH24 | CATTGAATGCAACTGGGGA |
| | | TCC | | | AGA |
| 135 | E45SaCas9KKH10 | TTGCCGCTGCCCAATGCCA | 145 | E45SaCas9KKH25 | GAACATTGAATGCAACTGG |
| | | TCC | | | GGA |
| 135 | E45SaCas9KKH10 | TTGCCGCTGCCCAATGCCA | 146 | E45SaCas9KKH26 | GCGGCAAACTGTTGTCAGA |
| | | TCC | | | ACA |
| 135 | E45SaCas9KKH10 | TTGCCGCTGCCCAATGCCA | 147 | E45SaCas9KKH27 | TTTTGGTATCTTACAGGAA |
| | | TCC | | | CTC |
| 136 | E45SaCas9KKH16 | AAACAGCTGTCAGACAGAA | 137 | E45SaCas9KKH17 | GAAGCCTGAATCTGCGGTG |
| | | AAA | | | GCA |
| 136 | E45SaCas9KKH16 | AAACAGCTGTCAGACAGAA | 138 | E45SaCas9KKH18 | GAAAAATTGGGAAGCCTGA |
| | | AAA | | | ATC |
| 136 | E45SaCas9KKH16 | AAACAGCTGTCAGACAGAA | 139 | E45SaCas9KKH19 | TCTACAGGAAAAATTGGGA |
| | | AAA | | | AGC |
| 136 | E45SaCas9KKH16 | AAACAGCTGTCAGACAGAA | 140 | E45SaCas9KKH20 | ACAGATGCCAGTATTCTAC |
| | | AAA | | | AGG |
| 136 | E45SaCas9KKH16 | AAACAGCTGTCAGACAGAA | 141 | E45SaCas9KKH21 | TCAGCAATCCTCAAAAACA |
| | | AAA | | | GAT |
| 136 | E45SaCas9KKH16 | AAACAGCTGTCAGACAGAA | 142 | E45SaCas9KKH22 | AATAATTCAGCAATCCTCA |
| | | AAA | | | AAA |
| 136 | E45SaCas9KKH16 | AAACAGCTGTCAGACAGAA | 143 | E45SaCas9KKH23 | GCAACTGGGGAAGAAATAA |
| | | AAA | | | TTC |
| 136 | E45SaCas9KKH16 | AAACAGCTGTCAGACAGAA | 144 | E45SaCas9KKH24 | CATTGAATGCAACTGGGGA |
| | | AAA | | | AGA |
| 136 | E45SaCas9KKH16 | AAACAGCTGTCAGACAGAA | 145 | E45SaCas9KKH25 | GAACATTGAATGCAACTGG |
| | | AAA | | | GGA |
| 136 | E45SaCas9KKH16 | AAACAGCTGTCAGACAGAA | 146 | E45SaCas9KKH26 | GCGGCAAACTGTTGTCAGA |
| | | AAA | | | ACA |
| 136 | E45SaCas9KKH16 | AAACAGCTGTCAGACAGAA | 147 | E45SaCas9KKH27 | TTTTGGTATCTTACAGGAA |
| | | AAA | | | CTC |
| 137 | E45SaCas9KKH17 | GAAGCCTGAATCTGCGGTG | 138 | E45SaCas9KKH18 | GAAAAATTGGGAAGCCTGA |
| | | GCA | | | ATC |
| 137 | E45SaCas9KKH17 | GAAGCCTGAATCTGCGGTG | 139 | E45SaCas9KKH19 | TCTACAGGAAAAATTGGGA |
| | | GCA | | | AGC |
| 137 | E45SaCas9KKH17 | GAAGCCTGAATCTGCGGTG | 140 | E45SaCas9KKH20 | ACAGATGCCAGTATTCTAC |
| | | GCA | | | AGG |
| 137 | E45SaCas9KKH17 | GAAGCCTGAATCTGCGGTG | 141 | E45SaCas9KKH21 | TCAGCAATCCTCAAAAACA |
| | | GCA | | | GAT |
| 137 | E45SaCas9KKH17 | GAAGCCTGAATCTGCGGTG | 142 | E45SaCas9KKH22 | AATAATTCAGCAATCCTCA |
| | | GCA | | | AAA |
| 137 | E45SaCas9KKH17 | GAAGCCTGAATCTGCGGTG | 143 | E45SaCas9KKH23 | GCAACTGGGGAAGAAATAA |
| | | GCA | | | TTC |
| 137 | E45SaCas9KKH17 | GAAGCCTGAATCTGCGGTG | 144 | E45SaCas9KKH24 | CATTGAATGCAACTGGGGA |
| | | GCA | | | AGA |
| 137 | E45SaCas9KKH17 | GAAGCCTGAATCTGCGGTG | 145 | E45SaCas9KKH25 | GAACATTGAATGCAACTGG |
| | | GCA | | | GGA |
| 137 | E45SaCas9KKH17 | GAAGCCTGAATCTGCGGTG | 146 | E45SaCas9KKH26 | GCGGCAAACTGTTGTCAGA |
| | | GCA | | | ACA |
| 137 | E45SaCas9KKH17 | GAAGCCTGAATCTGCGGTG | 147 | E45SaCas9KKH27 | TTTTGGTATCTTACAGGAA |
| | | GCA | | | CTC |
| 138 | E45SaCas9KKH18 | GAAAAATTGGGAAGCCTGA | 139 | E45SaCas9KKH19 | TCTACAGGAAAAATTGGGA |
| | | ATC | | | AGC |
| 138 | E45SaCas9KKH18 | GAAAAATTGGGAAGCCTGA | 140 | E45SaCas9KKH20 | ACAGATGCCAGTATTCTAC |
| | | ATC | | | AGG |
| 138 | E45SaCas9KKH18 | GAAAAATTGGGAAGCCTGA | 141 | E45SaCas9KKH21 | TCAGCAATCCTCAAAAACA |
| | | ATC | | | GAT |
| 138 | E45SaCas9KKH18 | GAAAAATTGGGAAGCCTGA | 142 | E45SaCas9KKH22 | AATAATTCAGCAATCCTCA |
| | | ATC | | | AAA |
| 138 | E45SaCas9KKH18 | GAAAAATTGGGAAGCCTGA | 143 | E45SaCas9KKH23 | GCAACTGGGGAAGAAATAA |
| | | ATC | | | TTC |
| 138 | E45SaCas9KKH18 | GAAAAATTGGGAAGCCTGA | 144 | E45SaCas9KKH24 | CATTGAATGCAACTGGGGA |
| | | ATC | | | AGA |
| 138 | E45SaCas9KKH18 | GAAAAATTGGGAAGCCTGA | 145 | E45SaCas9KKH25 | GAACATTGAATGCAACTGG |
| | | ATC | | | GGA |
| 138 | E45SaCas9KKH18 | GAAAAATTGGGAAGCCTGA | 146 | E45SaCas9KKH26 | GCGGCAAACTGTTGTCAGA |
| | | ATC | | | ACA |
| 138 | E45SaCas9KKH18 | GAAAAATTGGGAAGCCTGA | 147 | E45SaCas9KKH27 | TTTTGGTATCTTACAGGAA |
| | | ATC | | | CTC |
| 139 | E45SaCas9KKH19 | TCTACAGGAAAAATTGGGA | 140 | E45SaCas9KKH20 | ACAGATGCCAGTATTCTAC |
| | | AGC | | | AGG |
| 139 | E45SaCas9KKH19 | TCTACAGGAAAAATTGGGA | 141 | E45SaCas9KKH21 | TCAGCAATCCTCAAAAACA |
| | | AGC | | | GAT |
| 139 | E45SaCas9KKH19 | TCTACAGGAAAAATTGGGA | 142 | E45SaCas9KKH22 | AATAATTCAGCAATCCTCA |
| | | AGC | | | AAA |
| 139 | E45SaCas9KKH19 | TCTACAGGAAAAATTGGGA | 143 | E45SaCas9KKH23 | GCAACTGGGGAAGAAATAA |
| | | AGC | | | TTC |
| 139 | E45SaCas9KKH19 | TCTACAGGAAAAATTGGGA | 144 | E45SaCas9KKH24 | CATTGAATGCAACTGGGGA |
| | | AGC | | | AGA |
| 139 | E45SaCas9KKH19 | TCTACAGGAAAAATTGGGA | 145 | E45SaCas9KKH25 | GAACATTGAATGCAACTGG |
| | | AGC | | | GGA |
| 139 | E45SaCas9KKH19 | TCTACAGGAAAAATTGGGA | 146 | E45SaCas9KKH26 | GCGGCAAACTGTTGTCAGA |
| | | AGC | | | ACA |
| 139 | E45SaCas9KKH19 | TCTACAGGAAAAATTGGGA | 147 | E45SaCas9KKH27 | TTTTGGTATCTTACAGGAA |
| | | AGC | | | CTC |
| 140 | E45SaCas9KKH20 | ACAGATGCCAGTATTCTAC | 141 | E45SaCas9KKH21 | TCAGCAATCCTCAAAAACA |
| | | AGG | | | GAT |
| 140 | E45SaCas9KKH20 | ACAGATGCCAGTATTCTAC | 142 | E45SaCas9KKH22 | AATAATTCAGCAATCCTCA |
| | | AGG | | | AAA |
| 140 | E45SaCas9KKH20 | ACAGATGCCAGTATTCTAC | 143 | E45SaCas9KKH23 | GCAACTGGGGAAGAAATAA |
| | | AGG | | | TTC |
| 140 | E45SaCas9KKH20 | ACAGATGCCAGTATTCTAC | 144 | E45SaCas9KKH24 | CATTGAATGCAACTGGGGA |
| | | AGG | | | AGA |
| 140 | E45SaCas9KKH20 | ACAGATGCCAGTATTCTAC | 145 | E45SaCas9KKH25 | GAACATTGAATGCAACTGG |
| | | AGG | | | GGA |
| 140 | E45SaCas9KKH20 | ACAGATGCCAGTATTCTAC | 146 | E45SaCas9KKH26 | GCGGCAAACTGTTGTCAGA |
| | | AGG | | | ACA |
| 140 | E45SaCas9KKH20 | ACAGATGCCAGTATTCTAC | 147 | E45SaCas9KKH27 | TTTTGGTATCTTACAGGAA |
| | | AGG | | | CTC |
| 141 | E45SaCas9KKH21 | TCAGCAATCCTCAAAAACA | 142 | E45SaCas9KKH22 | AATAATTCAGCAATCCTCA |
| | | GAT | | | AAA |
| 141 | E45SaCas9KKH21 | TCAGCAATCCTCAAAAACA | 143 | E45SaCas9KKH23 | GCAACTGGGGAAGAAATAA |
| | | GAT | | | TTC |
| 141 | E45SaCas9KKH21 | TCAGCAATCCTCAAAAACA | 144 | E45SaCas9KKH24 | CATTGAATGCAACTGGGGA |
| | | GAT | | | AGA |
| 141 | E45SaCas9KKH21 | TCAGCAATCCTCAAAAACA | 145 | E45SaCas9KKH25 | GAACATTGAATGCAACTGG |
| | | GAT | | | GGA |
| 141 | E45SaCas9KKH21 | TCAGCAATCCTCAAAAACA | 146 | E45SaCas9KKH26 | GCGGCAAACTGTTGTCAGA |
| | | GAT | | | ACA |
| 141 | E45SaCas9KKH21 | TCAGCAATCCTCAAAAACA | 147 | E45SaCas9KKH27 | TTTTGGTATCTTACAGGAA |
| | | GAT | | | CTC |
| 142 | E45SaCas9KKH22 | AATAATTCAGCAATCCTCA | 143 | E45SaCas9KKH23 | GCAACTGGGGAAGAAATAA |
| | | AAA | | | TTC |
| 142 | E45SaCas9KKH22 | AATAATTCAGCAATCCTCA | 144 | E45SaCas9KKH24 | CATTGAATGCAACTGG GA |
| | | AAA | | | AGA |
| 142 | E45SaCas9KKH22 | AATAATTCAGCAATCCTCA | 145 | E45SaCas9KKH25 | GAACATTGAATGCAACTGG |
| | | AAA | | | GGA |
| 142 | E45SaCas9KKH22 | AATAATTCAGCAATCCTCA | 146 | E45SaCas9KKH26 | GCGGCAAACTGTTGTCAGA |
| | | AAA | | | ACA |
| 142 | E45SaCas9KKH22 | AATAATTCAGCAATCCTCA | 147 | E45SaCas9KKH27 | TTTTGGTATCTTACAGGAA |
| | | AAA | | | CTC |
| 143 | E45SaCas9KKH23 | GCAACTGGGGAAGAAATAA | 144 | E45SaCas9KKH24 | CATTGAATGCAACTGGGGA |
| | | TTC | | | AGA |
| 143 | E45SaCas9KKH23 | GCAACTGGGGAAGAAATAA | 145 | E45SaCas9KKH25 | GAACATTGAATGCAACTGG |
| | | TTC | | | GGA |
| 143 | E45SaCas9KKH23 | GCAACTGGGGAAGAAATAA | 146 | E45SaCas9KKH26 | GCGGCAAACTGTTGTCAGA |
| | | TTC | | | ACA |
| 143 | E45SaCas9KKH23 | GCAACTGGGGAAGAAATAA | 147 | E45SaCas9KKH27 | TTTTGGTATCTTACAGGAA |
| | | TTC | | | CTC |
| 144 | E45SaCas9KKH24 | CATTGAATGCAACTGGGGA | 145 | E45SaCas9KKH25 | GAACATTGAATGCAACTGG |
| | | AGA | | | GGA |
| 144 | E45SaCas9KKH24 | CATTGAATGCAACTGGGGA | 146 | E45SaCas9KKH26 | GCGGCAAACTGTTGTCAGA |
| | | AGA | | | ACA |
| 144 | E45SaCas9KKH24 | CATTGAATGCAACTGGGGA | 147 | E45SaCas9KKH27 | TTTTGGTATCTTACAGGAA |
| | | AGA | | | CTC |
| 145 | E45SaCas9KKH25 | GAACATTGAATGCAACTGG | 146 | E45SaCas9KKH26 | GCGGCAAACTGTTGTCAGA |
| | | GGA | | | ACA |
| 145 | E45SaCas9KKH25 | GAACATTGAATGCAACTGG | 147 | E45SaCas9KKH27 | TTTTGGTATCTTACAGGAA |
| | | GGA | | | CTC |
| 146 | E45SaCas9KKH26 | GCGGCAAACTGTTGTCAGA | 147 | E45SaCas9KKH27 | TTTTGGTATCTTACAGGAA |
| | | ACA | | | CTC |

TABLE 2C
(3) Exon 45 SluCas9 guides:

| Seq | | |
|---|---|---|
| ID | Guide ID or Guide | |
| No. | RNA Name | Guide Sequence |
| 209 | E45Slu1 | AGACCTCCTGCCACCGCAGATT |
| 210 | E45Slu2 | TCCCAATTTTTCCTGTAGAATA |
| 211 | E45Slu3 | TAGAATACTGGCATCTGTTTTT |
| 212 | E45Slu4 | TTTGCCGCTGCCCAATGCCATC |
| 213 | E45Slu7 | CTGTCAGACAGAAAAAAGAGGT |
| 214 | E45Slu8 | GCTGTCAGACAGAAAAAAGAGG |
| 215 | E45Slu9 | AACAGCTGTCAGACAGAAAAAA |
| 216 | E45Slu10 | AAGCCTGAATCTGCGGTGGCAG |
| 217 | E45Slu11 | GGGAAGCCTGAATCTGCGGTGG |
| 218 | E45Slu12 | AATTGGGAAGCCTGAATCTGCG |
| 219 | E45Slu13 | AAAAATTGGGAAGCCTGAATCT |
| 220 | E45Slu14 | GCCAGTATTCTACAGGAAAAAT |
| 221 | E45Slu15 | TGCCAGTATTCTACAGGAAAAA |
| 222 | E45Slu16 | AAAAACAGATGCCAGTATTCTA |
| 223 | E45Slu17 | TGTCAGAACATTGAATGCAACT |
| 224 | E45Slu18 | TTGTCAGAACATTGAATGCAAC |
| 225 | E45Slu19 | GTTGTCAGAACATTGAATGCAA |
| 226 | E45Slu20 | AACTCCAGGATGGCATTGGGCA |
| 227 | E45Slu21 | TACAGGAACTCCAGGATGGCAT |
| 228 | E45Slu22 | TTACAGGAACTCCAGGATGGCA |
| 229 | E45Slu23 | GGTATCTTACAGGAACTCCAGG |
| 230 | E45Slu24 | TTTTGGTATCTTACAGGAACTC |

TABLE 2D
(4) Exon 45 SluCas9 pairs:

| Guide 1 Seq ID No. | Guide 1 | Guide 1 Sequence | Guide 2 Seq ID No. | Guide 2 | Guide 2 Sequence |
|---|---|---|---|---|---|
| 209 | E45SL1 | AGACCTCCTGCCACCGCAGATT | 210 | E45SL2 | TCCCAATTTTTCCTGTAGAATA |
| 209 | E45SL1 | AGACCTCCTGCCACCGCAGATT | 211 | E45SL3 | TAGAATACTGGCATCTGTTTTT |
| 209 | E45SL1 | AGACCTCCTGCCACCGCAGATT | 212 | E45SL4 | TTTGCCGCTGCCCAATGCCATC |
| 209 | E45SL1 | AGACCTCCTGCCACCGCAGATT | 213 | E45SL7 | CTGTCAGACAGAAAAAAGAGGT |
| 209 | E45SL1 | AGACCTCCTGCCACCGCAGATT | 214 | E45SL8 | GCTGTCAGACAGAAAAAAGAGG |
| 209 | E45SL1 | AGACCTCCTGCCACCGCAGATT | 215 | E45SL9 | AACAGCTGTCAGACAGAAAAAA |
| 209 | E45SL1 | AGACCTCCTGCCACCGCAGATT | 216 | E45SL10 | AAGCCTGAATCTGCGGTGGCAG |
| 209 | E45SL1 | AGACCTCCTGCCACCGCAGATT | 217 | E45SL11 | GGGAAGCCTGAATCTGCGGTGG |
| 209 | E45SL1 | AGACCTCCTGCCACCGCAGATT | 218 | E45SL12 | AATTGGGAAGCCTGAATCTGCG |
| 209 | E45SL1 | AGACCTCCTGCCACCGCAGATT | 219 | E45SL13 | AAAAATTGGGAAGCCTGAATCT |
| 209 | E45SL1 | AGACCTCCTGCCACCGCAGATT | 220 | E45SL14 | GCCAGTATTCTACAGGAAAAAT |
| 209 | E45SL1 | AGACCTCCTGCCACCGCAGATT | 221 | E45SL15 | TGCCAGTATTCTACAGGAAAAA |
| 209 | E45SL1 | AGACCTCCTGCCACCGCAGATT | 222 | E45SL16 | AAAAACAGATGCCAGTATTCTA |
| 209 | E45SL1 | AGACCTCCTGCCACCGCAGATT | 223 | E45SL17 | TGTCAGAACATTGAATGCAACT |
| 209 | E45SL1 | AGACCTCCTGCCACCGCAGATT | 224 | E45SL18 | TTGTCAGAACATTGAATGCAAC |
| 209 | E45SL1 | AGACCTCCTGCCACCGCAGATT | 225 | E45SL19 | GTTGTCAGAACATTGAATGCAA |
| 209 | E45SL1 | AGACCTCCTGCCACCGCAGATT | 226 | E45SL20 | AACTCCAGGATGGCATTGGGCA |
| 209 | E45SL1 | AGACCTCCTGCCACCGCAGATT | 227 | E45SL21 | TACAGGAACTCCAGGATGGCAT |
| 209 | E45SL1 | AGACCTCCTGCCACCGCAGATT | 228 | E45SL22 | TTACAGGAACTCCAGGATGGCA |
| 209 | E45SL1 | AGACCTCCTGCCACCGCAGATT | 229 | E45SL23 | GGTATCTTACAGGAACTCCAGG |
| 209 | E45SL1 | AGACCTCCTGCCACCGCAGATT | 230 | E45SL24 | TTTTGGTATCTTACAGGAACTC |
| 210 | E45SL2 | TCCCAATTTTTCCTGTAGAATA | 211 | E45SL3 | TAGAATACTGGCATCTGTTTTT |
| 210 | E45SL2 | TCCCAATTTTTCCTGTAGAATA | 212 | E45SL4 | TTTGCCGCTGCCCAATGCCATC |
| 210 | E45SL2 | TCCCAATTTTTCCTGTAGAATA | 213 | E45SL7 | CTGTCAGACAGAAAAAAGAGGT |
| 210 | E45SL2 | TCCCAATTTTTCCTGTAGAATA | 214 | E45SL8 | GCTGTCAGACAGAAAAAAGAGG |
| 210 | E45SL2 | TCCCAATTTTTCCTGTAGAATA | 215 | E45SL9 | AACAGCTGTCAGACAGAAAAAA |
| 210 | E45SL2 | TCCCAATTTTTCCTGTAGAATA | 216 | E45SL10 | AAGCCTGAATCTGCGGTGGCAG |
| 210 | E45SL2 | TCCCAATTTTTCCTGTAGAATA | 217 | E45SL11 | GGGAAGCCTGAATCTGCGGTGG |
| 210 | E45SL2 | TCCCAATTTTTCCTGTAGAATA | 218 | E45SL12 | AATTGGGAAGCCTGAATCTGCG |
| 210 | E45SL2 | TCCCAATTTTTCCTGTAGAATA | 219 | E45SL13 | AAAAATTGGGAAGCCTGAATCT |
| 210 | E45SL2 | TCCCAATTTTTCCTGTAGAATA | 220 | E45SL14 | GCCAGTATTCTACAGGAAAAAT |
| 210 | E45SL2 | TCCCAATTTTTCCTGTAGAATA | 221 | E45SL15 | TGCCAGTATTCTACAGGAAAAA |
| 210 | E45SL2 | TCCCAATTTTTCCTGTAGAATA | 222 | E45SL16 | AAAAACAGATGCCAGTATTCTA |
| 210 | E45SL2 | TCCCAATTTTTCCTGTAGAATA | 223 | E45SL17 | TGTCAGAACATTGAATGCAACT |
| 210 | E45SL2 | TCCCAATTTTTCCTGTAGAATA | 224 | E45SL18 | TTGTCAGAACATTGAATGCAAC |
| 210 | E45SL2 | TCCCAATTTTTCCTGTAGAATA | 225 | E45SL19 | GTTGTCAGAACATTGAATGCAA |
| 210 | E45SL2 | TCCCAATTTTTCCTGTAGAATA | 226 | E45SL20 | AACTCCAGGATGGCATTGGGCA |
| 210 | E45SL2 | TCCCAATTTTTCCTGTAGAATA | 227 | E45SL21 | TACAGGAACTCCAGGATGGCAT |
| 210 | E45SL2 | TCCCAATTTTTCCTGTAGAATA | 228 | E45SL22 | TTACAGGAACTCCAGGATGGCA |
| 210 | E45SL2 | TCCCAATTTTTCCTGTAGAATA | 229 | E45SL23 | GGTATCTTACAGGAACTCCAGG |
| 210 | E45SL2 | TCCCAATTTTTCCTGTAGAATA | 230 | E45SL24 | TTTTGGTATCTTACAGGAACTC |
| 211 | E45SL3 | TAGAATACTGGCATCTGTTTTT | 212 | E45SL4 | TTTGCCGCTGCCCAATGCCATC |
| 211 | E45SL3 | TAGAATACTGGCATCTGTTTTT | 213 | E45SL7 | CTGTCAGACAGAAAAAAGAGGT |
| 211 | E45SL3 | TAGAATACTGGCATCTGTTTTT | 214 | E45SL8 | GCTGTCAGACAGAAAAAAGAGG |
| 211 | E45SL3 | TAGAATACTGGCATCTGTTTTT | 215 | E45SL9 | AACAGCTGTCAGACAGAAAAAA |
| 211 | E45SL3 | TAGAATACTGGCATCTGTTTTT | 216 | E45SL10 | AAGCCTGAATCTGCGGTGGCAG |
| 211 | E45SL3 | TAGAATACTGGCATCTGTTTTT | 217 | E45SL11 | GGGAAGCCTGAATCTGCGGTGG |
| 211 | E45SL3 | TAGAATACTGGCATCTGTTTTT | 218 | E45SL12 | AATTGGGAAGCCTGAATCTGCG |
| 211 | E45SL3 | TAGAATACTGGCATCTGTTTTT | 219 | E45SL13 | AAAAATTGGGAAGCCTGAATCT |
| 211 | E45SL3 | TAGAATACTGGCATCTGTTTTT | 220 | E45SL14 | GCCAGTATTCTACAGGAAAAAT |
| 211 | E45SL3 | TAGAATACTGGCATCTGTTTTT | 221 | E45SL15 | TGCCAGTATTCTACAGGAAAAA |
| 211 | E45SL3 | TAGAATACTGGCATCTGTTTTT | 222 | E45SL16 | AAAAACAGATGCCAGTATTCTA |
| 211 | E45SL3 | TAGAATACTGGCATCTGTTTTT | 223 | E45SL17 | TGTCAGAACATTGAATGCAACT |
| 211 | E45SL3 | TAGAATACTGGCATCTGTTTTT | 224 | E45SL18 | TTGTCAGAACATTGAATGCAAC |
| 211 | E45SL3 | TAGAATACTGGCATCTGTTTTT | 225 | E45SL19 | GTTGTCAGAACATTGAATGCAA |
| 211 | E45SL3 | TAGAATACTGGCATCTGTTTTT | 226 | E45SL20 | AACTCCAGGATGGCATTGGGCA |
| 211 | E45SL3 | TAGAATACTGGCATCTGTTTTT | 227 | E45SL21 | TACAGGAACTCCAGGATGGCAT |
| 211 | E45SL3 | TAGAATACTGGCATCTGTTTTT | 228 | E45SL22 | TTACAGGAACTCCAGGATGGCA |
| 211 | E45SL3 | TAGAATACTGGCATCTGTTTTT | 229 | E45SL23 | GGTATCTTACAGGAACTCCAGG |
| 211 | E45SL3 | TAGAATACTGGCATCTGTTTTT | 230 | E45SL24 | TTTTGGTATCTTACAGGAACTC |
| 212 | E45SL4 | TTTGCCGCTGCCCAATGCCATC | 213 | E45SL7 | CTGTCAGACAGAAAAAAGAGGT |
| 212 | E45SL4 | TTTGCCGCTGCCCAATGCCATC | 214 | E45SL8 | GCTGTCAGACAGAAAAAAGAGG |
| 212 | E45SL4 | TTTGCCGCTGCCCAATGCCATC | 215 | E45SL9 | AACAGCTGTCAGACAGAAAAAA |
| 212 | E45SL4 | TTTGCCGCTGCCCAATGCCATC | 216 | E45SL10 | AAGCCTGAATCTGCGGTGGCAG |
| 212 | E45SL4 | TTTGCCGCTGCCCAATGCCATC | 217 | E45SL11 | GGGAAGCCTGAATCTGCGGTGG |
| 212 | E45SL4 | TTTGCCGCTGCCCAATGCCATC | 218 | E45SL12 | AATTGGGAAGCCTGAATCTGCG |
| 212 | E45SL4 | TTTGCCGCTGCCCAATGCCATC | 219 | E45SL13 | AAAAATTGGGAAGCCTGAATCT |
| 212 | E45SL4 | TTTGCCGCTGCCCAATGCCATC | 220 | E45SL14 | GCCAGTATTCTACAGGAAAAAT |
| 212 | E45SL4 | TTTGCCGCTGCCCAATGCCATC | 221 | E45SL15 | TGCCAGTATTCTACAGGAAAAA |
| 212 | E45SL4 | TTTGCCGCTGCCCAATGCCATC | 222 | E45SL16 | AAAAACAGATGCCAGTATTCTA |
| 212 | E45SL4 | TTTGCCGCTGCCCAATGCCATC | 223 | E45SL17 | TGTCAGAACATTGAATGCAACT |
| 212 | E45SL4 | TTTGCCGCTGCCCAATGCCATC | 224 | E45SL18 | TTGTCAGAACATTGAATGCAAC |
| 212 | E45SL4 | TTTGCCGCTGCCCAATGCCATC | 225 | E45SL19 | GTTGTCAGAACATTGAATGCAA |
| 212 | E45SL4 | TTTGCCGCTGCCCAATGCCATC | 226 | E45SL20 | AACTCCAGGATGGCATTGGGCA |
| 212 | E45SL4 | TTTGCCGCTGCCCAATGCCATC | 227 | E45SL21 | TACAGGAACTCCAGGATGGCAT |
| 212 | E45SL4 | TTTGCCGCTGCCCAATGCCATC | 228 | E45SL22 | TTACAGGAACTCCAGGATGGCA |
| 212 | E45SL4 | TTTGCCGCTGCCCAATGCCATC | 229 | E45SL23 | GGTATCTTACAGGAACTCCAGG |
| 212 | E45SL4 | TTTGCCGCTGCCCAATGCCATC | 230 | E45SL24 | TTTTGGTATCTTACAGGAACTC |
| 213 | E45SL7 | CTGTCAGACAGAAAAAAGAGGT | 214 | E45SL8 | GCTGTCAGACAGAAAAAAGAGG |
| 213 | E45SL7 | CTGTCAGACAGAAAAAAGAGGT | 215 | E45SL9 | AACAGCTGTCAGACAGAAAAAA |
| 213 | E45SL7 | CTGTCAGACAGAAAAAAGAGGT | 216 | E45SL10 | AAGCCTGAATCTGCGGTGGCAG |
| 213 | E45SL7 | CTGTCAGACAGAAAAAAGAGGT | 217 | E45SL11 | GGGAAGCCTGAATCTGCGGTGG |
| 213 | E45SL7 | CTGTCAGACAGAAAAAAGAGGT | 218 | E45SL12 | AATTGGGAAGCCTGAATCTGCG |
| 213 | E45SL7 | CTGTCAGACAGAAAAAAGAGGT | 219 | E45SL13 | AAAAATTGGGAAGCCTGAATCT |
| 213 | E45SL7 | CTGTCAGACAGAAAAAAGAGGT | 220 | E45SL14 | GCCAGTATTCTACAGGAAAAAT |
| 213 | E45SL7 | CTGTCAGACAGAAAAAAGAGGT | 221 | E45SL15 | TGCCAGTATTCTACAGGAAAAA |
| 213 | E45SL7 | CTGTCAGACAGAAAAAAGAGGT | 222 | E45SL16 | AAAAACAGATGCCAGTATTCTA |
| 213 | E45SL7 | CTGTCAGACAGAAAAAAGAGGT | 223 | E45SL17 | TGTCAGAACATTGAATGCAACT |
| 213 | E45SL7 | CTGTCAGACAGAAAAAAGAGGT | 224 | E45SL18 | TTGTCAGAACATTGAATGCAAC |
| 213 | E45SL7 | CTGTCAGACAGAAAAAAGAGGT | 225 | E45SL19 | GTTGTCAGAACATTGAATGCAA |
| 213 | E45SL7 | CTGTCAGACAGAAAAAAGAGGT | 226 | E45SL20 | AACTCCAGGATGGCATTGGGCA |
| 213 | E45SL7 | CTGTCAGACAGAAAAAAGAGGT | 227 | E45SL21 | TACAGGAACTCCAGGATGGCAT |
| 213 | E45SL7 | CTGTCAGACAGAAAAAAGAGGT | 228 | E45SL22 | TTACAGGAACTCCAGGATGGCA |
| 213 | E45SL7 | CTGTCAGACAGAAAAAAGAGGT | 229 | E45SL23 | GGTATCTTACAGGAACTCCAGG |
| 213 | E45SL7 | CTGTCAGACAGAAAAAAGAGGT | 230 | E45SL24 | TTTTGGTATCTTACAGGAACTC |
| 214 | E45SL8 | GCTGTCAGACAGAAAAAAGAGG | 215 | E45SL9 | AACAGCTGTCAGACAGAAAAAA |
| 214 | E45SL8 | GCTGTCAGACAGAAAAAAGAGG | 216 | E45SL10 | AAGCCTGAATCTGCGGTGGCAG |
| 214 | E45SL8 | GCTGTCAGACAGAAAAAAGAGG | 217 | E45SL11 | GGGAAGCCTGAATCTGCGGTGG |
| 214 | E45SL8 | GCTGTCAGACAGAAAAAAGAGG | 218 | E45SL12 | AATTGGGAAGCCTGAATCTGCG |
| 214 | E45SL8 | GCTGTCAGACAGAAAAAAGAGG | 219 | E45SL13 | AAAAATTGGGAAGCCTGAATCT |
| 214 | E45SL8 | GCTGTCAGACAGAAAAAAGAGG | 220 | E45SL14 | GCCAGTATTCTACAGGAAAAAT |
| 214 | E45SL8 | GCTGTCAGACAGAAAAAAGAGG | 221 | E45SL15 | TGCCAGTATTCTACAGGAAAAA |
| 214 | E45SL8 | GCTGTCAGACAGAAAAAAGAGG | 222 | E45SL16 | AAAAACAGATGCCAGTATTCTA |
| 214 | E45SL8 | GCTGTCAGACAGAAAAAAGAGG | 223 | E45SL17 | TGTCAGAACATTGAATGCAACT |
| 214 | E45SL8 | GCTGTCAGACAGAAAAAAGAGG | 224 | E45SL18 | TTGTCAGAACATTGAATGCAAC |
| 214 | E45SL8 | GCTGTCAGACAGAAAAAAGAGG | 225 | E45SL19 | GTTGTCAGAACATTGAATGCAA |
| 214 | E45SL8 | GCTGTCAGACAGAAAAAAGAGG | 226 | E45SL20 | AACTCCAGGATGGCATTGGGCA |
| 214 | E45SL8 | GCTGTCAGACAGAAAAAAGAGG | 227 | E45SL21 | TACAGGAACTCCAGGATGGCAT |
| 214 | E45SL8 | GCTGTCAGACAGAAAAAAGAGG | 228 | E45SL22 | TTACAGGAACTCCAGGATGGCA |
| 214 | E45SL8 | GCTGTCAGACAGAAAAAAGAGG | 229 | E45SL23 | GGTATCTTACAGGAACTCCAGG |
| 214 | E45SL8 | GCTGTCAGACAGAAAAAAGAGG | 230 | E45SL24 | TTTTGGTATCTTACAGGAACTC |
| 215 | E45SL9 | AACAGCTGTCAGACAGAAAAAA | 216 | E45SL10 | AAGCCTGAATCTGCGGTGGCAG |
| 215 | E45SL9 | AACAGCTGTCAGACAGAAAAAA | 217 | E45SL11 | GGGAAGCCTGAATCTGCGGTGG |
| 215 | E45SL9 | AACAGCTGTCAGACAGAAAAAA | 218 | E45SL12 | AATTGGGAAGCCTGAATCTGCG |
| 215 | E45SL9 | AACAGCTGTCAGACAGAAAAAA | 219 | E45SL13 | AAAAATTGGGAAGCCTGAATCT |
| 215 | E45SL9 | AACAGCTGTCAGACAGAAAAAA | 220 | E45SL14 | GCCAGTATTCTACAGGAAAAAT |
| 215 | E45SL9 | AACAGCTGTCAGACAGAAAAAA | 221 | E45SL15 | TGCCAGTATTCTACAGGAAAAA |
| 215 | E45SL9 | AACAGCTGTCAGACAGAAAAAA | 222 | E45SL16 | AAAAACAGATGCCAGTATTCTA |
| 215 | E45SL9 | AACAGCTGTCAGACAGAAAAAA | 223 | E45SL17 | TGTCAGAACATTGAATGCAACT |
| 215 | E45SL9 | AACAGCTGTCAGACAGAAAAAA | 224 | E45SL18 | TTGTCAGAACATTGAATGCAAC |
| 215 | E45SL9 | AACAGCTGTCAGACAGAAAAAA | 225 | E45SL19 | GTTGTCAGAACATTGAATGCAA |
| 215 | E45SL9 | AACAGCTGTCAGACAGAAAAAA | 226 | E45SL20 | AACTCCAGGATGGCATTGGGCA |
| 215 | E45SL9 | AACAGCTGTCAGACAGAAAAAA | 227 | E45SL21 | TACAGGAACTCCAGGATGGCAT |
| 215 | E45SL9 | AACAGCTGTCAGACAGAAAAAA | 228 | E45SL22 | TTACAGGAACTCCAGGATGGCA |
| 215 | E45SL9 | AACAGCTGTCAGACAGAAAAAA | 229 | E45SL23 | GGTATCTTACAGGAACTCCAGG |
| 215 | E45SL9 | AACAGCTGTCAGACAGAAAAAA | 230 | E45SL24 | TTTTGGTATCTTACAGGAACTC |
| 216 | E45SL10 | AAGCCTGAATCTGCGGTGGCAG | 217 | E45SL11 | GGGAAGCCTGAATCTGCGGTGG |
| 216 | E45SL10 | AAGCCTGAATCTGCGGTGGCAG | 218 | E45SL12 | AATTGGGAAGCCTGAATCTGCG |
| 216 | E45SL10 | AAGCCTGAATCTGCGGTGGCAG | 219 | E45SL13 | AAAAATTGGGAAGCCTGAATCT |
| 216 | E45SL10 | AAGCCTGAATCTGCGGTGGCAG | 220 | E45SL14 | GCCAGTATTCTACAGGAAAAAT |
| 216 | E45SL10 | AAGCCTGAATCTGCGGTGGCAG | 221 | E45SL15 | TGCCAGTATTCTACAGGAAAAA |
| 216 | E45SL10 | AAGCCTGAATCTGCGGTGGCAG | 222 | E45SL16 | AAAAACAGATGCCAGTATTCTA |
| 216 | E45SL10 | AAGCCTGAATCTGCGGTGGCAG | 223 | E45SL17 | TGTCAGAACATTGAATGCAACT |
| 216 | E45SL10 | AAGCCTGAATCTGCGGTGGCAG | 224 | E45SL18 | TTGTCAGAACATTGAATGCAAC |
| 216 | E45SL10 | AAGCCTGAATCTGCGGTGGCAG | 225 | E45SL19 | GTTGTCAGAACATTGAATGCAA |
| 216 | E45SL10 | AAGCCTGAATCTGCGGTGGCAG | 226 | E45SL20 | AACTCCAGGATGGCATTGGGCA |
| 216 | E45SL10 | AAGCCTGAATCTGCGGTGGCAG | 227 | E45SL21 | TACAGGAACTCCAGGATGGCAT |
| 216 | E45SL10 | AAGCCTGAATCTGCGGTGGCAG | 228 | E45SL22 | TTACAGGAACTCCAGGATGGCA |
| 216 | E45SL10 | AAGCCTGAATCTGCGGTGGCAG | 229 | E45SL23 | GGTATCTTACAGGAACTCCAGG |
| 216 | E45SL10 | AAGCCTGAATCTGCGGTGGCAG | 230 | E45SL24 | TTTTGGTATCTTACAGGAACTC |
| 217 | E45SL11 | GGGAAGCCTGAATCTGCGGTGG | 218 | E45SL12 | AATTGGGAAGCCTGAATCTGCG |
| 217 | E45SL11 | GGGAAGCCTGAATCTGCGGTGG | 219 | E45SL13 | AAAAATTGGGAAGCCTGAATCT |
| 217 | E45SL11 | GGGAAGCCTGAATCTGCGGTGG | 220 | E45SL14 | GCCAGTATTCTACAGGAAAAAT |
| 217 | E45SL11 | GGGAAGCCTGAATCTGCGGTGG | 221 | E45SL15 | TGCCAGTATTCTACAGGAAAAA |
| 217 | E45SL11 | GGGAAGCCTGAATCTGCGGTGG | 222 | E45SL16 | AAAAACAGATGCCAGTATTCTA |
| 217 | E45SL11 | GGGAAGCCTGAATCTGCGGTGG | 223 | E45SL17 | TGTCAGAACATTGAATGCAACT |
| 217 | E45SL11 | GGGAAGCCTGAATCTGCGGTGG | 224 | E45SL18 | TTGTCAGAACATTGAATGCAAC |
| 217 | E45SL11 | GGGAAGCCTGAATCTGCGGTGG | 225 | E45SL19 | GTTGTCAGAACATTGAATGCAA |
| 217 | E45SL11 | GGGAAGCCTGAATCTGCGGTGG | 226 | E45SL20 | AACTCCAGGATGGCATTGGGCA |
| 217 | E45SL11 | GGGAAGCCTGAATCTGCGGTGG | 227 | E45SL21 | TACAGGAACTCCAGGATGGCAT |
| 217 | E45SL11 | GGGAAGCCTGAATCTGCGGTGG | 228 | E45SL22 | TTACAGGAACTCCAGGATGGCA |
| 217 | E45SL11 | GGGAAGCCTGAATCTGCGGTGG | 229 | E45SL23 | GGTATCTTACAGGAACTCCAGG |
| 217 | E45SL11 | GGGAAGCCTGAATCTGCGGTGG | 230 | E45SL24 | TTTTGGTATCTTACAGGAACTC |
| 218 | E45SL12 | AATTGGGAAGCCTGAATCTGCG | 219 | E45SL13 | AAAAATTGGGAAGCCTGAATCT |
| 218 | E45SL12 | AATTGGGAAGCCTGAATCTGCG | 220 | E45SL14 | GCCAGTATTCTACAGGAAAAAT |
| 218 | E45SL12 | AATTGGGAAGCCTGAATCTGCG | 221 | E45SL15 | TGCCAGTATTCTACAGGAAAAA |
| 218 | E45SL12 | AATTGGGAAGCCTGAATCTGCG | 222 | E45SL16 | AAAAACAGATGCCAGTATTCTA |
| 218 | E45SL12 | AATTGGGAAGCCTGAATCTGCG | 223 | E45SL17 | TGTCAGAACATTGAATGCAACT |
| 218 | E45SL12 | AATTGGGAAGCCTGAATCTGCG | 224 | E45SL18 | TTGTCAGAACATTGAATGCAAC |
| 218 | E45SL12 | AATTGGGAAGCCTGAATCTGCG | 225 | E45SL19 | GTTGTCAGAACATTGAATGCAA |
| 218 | E45SL12 | AATTGGGAAGCCTGAATCTGCG | 226 | E45SL20 | AACTCCAGGATGGCATTGGGCA |
| 218 | E45SL12 | AATTGGGAAGCCTGAATCTGCG | 227 | E45SL21 | TACAGGAACTCCAGGATGGCAT |
| 218 | E45SL12 | AATTGGGAAGCCTGAATCTGCG | 228 | E45SL22 | TTACAGGAACTCCAGGATGGCA |
| 218 | E45SL12 | AATTGGGAAGCCTGAATCTGCG | 229 | E45SL23 | GGTATCTTACAGGAACTCCAGG |
| 218 | E45SL12 | AATTGGGAAGCCTGAATCTGCG | 230 | E45SL24 | TTTTGGTATCTTACAGGAACTC |
| 219 | E45SL13 | AAAAATTGGGAAGCCTGAATCT | 220 | E45SL14 | GCCAGTATTCTACAGGAAAAAT |
| 219 | E45SL13 | AAAAATTGGGAAGCCTGAATCT | 221 | E45SL15 | TGCCAGTATTCTACAGGAAAAA |
| 219 | E45SL13 | AAAAATTGGGAAGCCTGAATCT | 222 | E45SL16 | AAAAACAGATGCCAGTATTCTA |
| 219 | E45SL13 | AAAAATTGGGAAGCCTGAATCT | 223 | E45SL17 | TGTCAGAACATTGAATGCAACT |
| 219 | E45SL13 | AAAAATTGGGAAGCCTGAATCT | 224 | E45SL18 | TTGTCAGAACATTGAATGCAAC |
| 219 | E45SL13 | AAAAATTGGGAAGCCTGAATCT | 225 | E45SL19 | GTTGTCAGAACATTGAATGCAA |
| 219 | E45SL13 | AAAAATTGGGAAGCCTGAATCT | 226 | E45SL20 | AACTCCAGGATGGCATTGGGCA |
| 219 | E45SL13 | AAAAATTGGGAAGCCTGAATCT | 227 | E45SL21 | TACAGGAACTCCAGGATGGCAT |
| 219 | E45SL13 | AAAAATTGGGAAGCCTGAATCT | 228 | E45SL22 | TTACAGGAACTCCAGGATGGCA |
| 219 | E45SL13 | AAAAATTGGGAAGCCTGAATCT | 229 | E45SL23 | GGTATCTTACAGGAACTCCAGG |
| 219 | E45SL13 | AAAAATTGGGAAGCCTGAATCT | 230 | E45SL24 | TTTTGGTATCTTACAGGAACTC |
| 220 | E45SL14 | GCCAGTATTCTACAGGAAAAAT | 221 | E45SL15 | TGCCAGTATTCTACAGGAAAAA |
| 220 | E45SL14 | GCCAGTATTCTACAGGAAAAAT | 222 | E45SL16 | AAAAACAGATGCCAGTATTCTA |
| 220 | E45SL14 | GCCAGTATTCTACAGGAAAAAT | 223 | E45SL17 | TGTCAGAACATTGAATGCAACT |
| 220 | E45SL14 | GCCAGTATTCTACAGGAAAAAT | 224 | E45SL18 | TTGTCAGAACATTGAATGCAAC |
| 220 | E45SL14 | GCCAGTATTCTACAGGAAAAAT | 225 | E45SL19 | GTTGTCAGAACATTGAATGCAA |
| 220 | E45SL14 | GCCAGTATTCTACAGGAAAAAT | 226 | E45SL20 | AACTCCAGGATGGCATTGGGCA |
| 220 | E45SL14 | GCCAGTATTCTACAGGAAAAAT | 227 | E45SL21 | TACAGGAACTCCAGGATGGCAT |
| 220 | E45SL14 | GCCAGTATTCTACAGGAAAAAT | 228 | E45SL22 | TTACAGGAACTCCAGGATGGCA |
| 220 | E45SL14 | GCCAGTATTCTACAGGAAAAAT | 229 | E45SL23 | GGTATCTTACAGGAACTCCAGG |
| 220 | E45SL14 | GCCAGTATTCTACAGGAAAAAT | 230 | E45SL24 | TTTTGGTATCTTACAGGAACTC |
| 221 | E45SL15 | TGCCAGTATTCTACAGGAAAAA | 222 | E45SL16 | AAAAACAGATGCCAGTATTCTA |
| 221 | E45SL15 | TGCCAGTATTCTACAGGAAAAA | 223 | E45SL17 | TGTCAGAACATTGAATGCAACT |
| 221 | E45SL15 | TGCCAGTATTCTACAGGAAAAA | 224 | E45SL18 | TTGTCAGAACATTGAATGCAAC |
| 221 | E45SL15 | TGCCAGTATTCTACAGGAAAAA | 225 | E45SL19 | GTTGTCAGAACATTGAATGCAA |
| 221 | E45SL15 | TGCCAGTATTCTACAGGAAAAA | 226 | E45SL20 | AACTCCAGGATGGCATTGGGCA |
| 221 | E45SL15 | TGCCAGTATTCTACAGGAAAAA | 227 | E45SL21 | TACAGGAACTCCAGGATGGCAT |
| 221 | E45SL15 | TGCCAGTATTCTACAGGAAAAA | 228 | E45SL22 | TTACAGGAACTCCAGGATGGCA |
| 221 | E45SL15 | TGCCAGTATTCTACAGGAAAAA | 229 | E45SL23 | GGTATCTTACAGGAACTCCAGG |
| 221 | E45SL15 | TGCCAGTATTCTACAGGAAAAA | 230 | E45SL24 | TTTTGGTATCTTACAGGAACTC |
| 222 | E45SL16 | AAAAACAGATGCCAGTATTCTA | 223 | E45SL17 | TGTCAGAACATTGAATGCAACT |
| 222 | E45SL16 | AAAAACAGATGCCAGTATTCTA | 224 | E45SL18 | TTGTCAGAACATTGAATGCAAC |
| 222 | E45SL16 | AAAAACAGATGCCAGTATTCTA | 225 | E45SL19 | GTTGTCAGAACATTGAATGCAA |
| 222 | E45SL16 | AAAAACAGATGCCAGTATTCTA | 226 | E45SL20 | AACTCCAGGATGGCATTGGGCA |
| 222 | E45SL16 | AAAAACAGATGCCAGTATTCTA | 227 | E45SL21 | TACAGGAACTCCAGGATGGCAT |
| 222 | E45SL16 | AAAAACAGATGCCAGTATTCTA | 228 | E45SL22 | TTACAGGAACTCCAGGATGGCA |
| 222 | E45SL16 | AAAAACAGATGCCAGTATTCTA | 229 | E45SL23 | GGTATCTTACAGGAACTCCAGG |
| 222 | E45SL16 | AAAAACAGATGCCAGTATTCTA | 230 | E45SL24 | TTTTGGTATCTTACAGGAACTC |
| 223 | E45SL17 | TGTCAGAACATTGAATGCAACT | 224 | E45SL18 | TTGTCAGAACATTGAATGCAAC |
| 223 | E45SL17 | TGTCAGAACATTGAATGCAACT | 225 | E45SL19 | GTTGTCAGAACATTGAATGCAA |
| 223 | E45SL17 | TGTCAGAACATTGAATGCAACT | 226 | E45SL20 | AACTCCAGGATGGCATTGGGCA |
| 223 | E45SL17 | TGTCAGAACATTGAATGCAACT | 227 | E45SL21 | TACAGGAACTCCAGGATGGCAT |
| 223 | E45SL17 | TGTCAGAACATTGAATGCAACT | 228 | E45SL22 | TTACAGGAACTCCAGGATGGCA |
| 223 | E45SL17 | TGTCAGAACATTGAATGCAACT | 229 | E45SL23 | GGTATCTTACAGGAACTCCAGG |
| 223 | E45SL17 | TGTCAGAACATTGAATGCAACT | 230 | E45SL24 | TTTTGGTATCTTACAGGAACTC |
| 224 | E45SL18 | TTGTCAGAACATTGAATGCAAC | 225 | E45SL19 | GTTGTCAGAACATTGAATGCAA |
| 224 | E45SL18 | TTGTCAGAACATTGAATGCAAC | 226 | E45SL20 | AACTCCAGGATGGCATTGGGCA |
| 224 | E45SL18 | TTGTCAGAACATTGAATGCAAC | 227 | E45SL21 | TACAGGAACTCCAGGATGGCAT |
| 224 | E45SL18 | TTGTCAGAACATTGAATGCAAC | 228 | E45SL22 | TTACAGGAACTCCAGGATGGCA |
| 224 | E45SL18 | TTGTCAGAACATTGAATGCAAC | 229 | E45SL23 | GGTATCTTACAGGAACTCCAGG |
| 224 | E45SL18 | TTGTCAGAACATTGAATGCAAC | 230 | E45SL24 | TTTTGGTATCTTACAGGAACTC |
| 225 | E45SL19 | GTTGTCAGAACATTGAATGCAA | 226 | E45SL20 | AACTCCAGGATGGCATTGGGCA |
| 225 | E45SL19 | GTTGTCAGAACATTGAATGCAA | 227 | E45SL21 | TACAGGAACTCCAGGATGGCAT |
| 225 | E45SL19 | GTTGTCAGAACATTGAATGCAA | 228 | E45SL22 | TTACAGGAACTCCAGGATGGCA |
| 225 | E45SL19 | GTTGTCAGAACATTGAATGCAA | 229 | E45SL23 | GGTATCTTACAGGAACTCCAGG |
| 225 | E45SL19 | GTTGTCAGAACATTGAATGCAA | 230 | E45SL24 | TTTTGGTATCTTACAGGAACTC |
| 226 | E45SL20 | AACTCCAGGATGGCATTGGGCA | 227 | E45SL21 | TACAGGAACTCCAGGATGGCAT |
| 226 | E45SL20 | AACTCCAGGATGGCATTGGGCA | 228 | E45SL22 | TTACAGGAACTCCAGGATGGCA |
| 226 | E45SL20 | AACTCCAGGATGGCATTGGGCA | 229 | E45SL23 | GGTATCTTACAGGAACTCCAGG |
| 226 | E45SL20 | AACTCCAGGATGGCATTGGGCA | 230 | E45SL24 | TTTTGGTATCTTACAGGAACTC |
| 227 | E45SL21 | TACAGGAACTCCAGGATGGCAT | 228 | E45SL22 | TTACAGGAACTCCAGGATGGCA |
| 227 | E45SL21 | TACAGGAACTCCAGGATGGCAT | 229 | E45SL23 | GGTATCTTACAGGAACTCCAGG |
| 227 | E45SL21 | TACAGGAACTCCAGGATGGCAT | 230 | E45SL24 | TTTTGGTATCTTACAGGAACTC |
| 228 | E45SL22 | TTACAGGAACTCCAGGATGGCA | 229 | E45SL23 | GGTATCTTACAGGAACTCCAGG |
| 228 | E45SL22 | TTACAGGAACTCCAGGATGGCA | 230 | E45SL24 | TTTTGGTATCTTACAGGAACTC |
| 229 | E45SL23 | GGTATCTTACAGGAACTCCAGG | 230 | E45SL24 | TTTTGGTATCTTACAGGAACTC |

### c) Exon 50:

TABLE 3A
(1) Exon 50 SaCas9 guides:

| Seq | Guide ID or | |
|---|---|---|
| ID No. | Guide RNA Name | Guide Sequence |
| 148 | E50SaCas9KKH1 | GGGATCCAGTATACTTACAGGC |
| 149 | E50SaCas9KKH2 | ATCCAGTATACTTACAGGCTCC |
| 150 | E50SaCas9KKH3 | CAGTATACTTACAGGCTCCAAT |
| 151 | E50SaCas9KKH4 | ATACTTACAGGCTCCAATAGTG |
| 152 | E50SaCas9KKH5 | CCAATAGTGGTCAGTCCAGGAG |
| 153 | E50SaCas9KKH6 | AGGCTGCTTTGCCCTCAGCTCT |
| 154 | E50SaCas9KKH7 | TTTGCCCTCAGCTCTTGAAGTA |
| 155 | E50SaCas9KKH8 | TTTACCGCCTTCCACTCAGAGC |
| 156 | E50SaCas9KKH10 | GGACTGACCACTATTGGAGCCT |
| 157 | E50SaCas9KKH11 | AAGATCTGAGCTCTGAGTGGAA |
| 158 | E50SaCas9KKH12 | AGGAAGTTAGAAGATCTGAGCT |
| 159 | E50SaCas9KKH13 | TTTTCTGTTAAAGAGGAAGTTA |

TABLE 3B
(2) Exon 50 SaCas9 pairs:

| Guide 1 | | | Guide 2 | | |
|---|---|---|---|---|---|
| Seq ID No. | Guide 1 | Guide 1 Sequence | Seq ID No. | Guide 2 | Guide 2 Sequence |
| 148 | E50SaCas9KKH | GGGATCCAGTATACTTACA | 149 | E50SaCas9KKH | ATCCAGTATACTTACAGGC |
| | 1 | GGC | | 2 | TCC |
| 148 | E50SaCas9KKH | GGGATCCAGTATACTTACA | 150 | E50SaCas9KKH | CAGTATACTTACAGGCTCC |
| | 1 | GGC | | 3 | AAT |
| 148 | E50SaCas9KKH | GGGATCCAGTATACTTACA | 151 | E50SaCas9KKH | ATACTTACAGGCTCCAATA |
| | 1 | GGC | | 4 | GTG |
| 148 | E50SaCas9KKH | GGGATCCAGTATACTTACA | 152 | E50SaCas9KKH | CCAATAGTGGTCAGTCCAG |
| | 1 | GGC | | 5 | GAG |
| 148 | E50SaCas9KKH | GGGATCCAGTATACTTACA | 153 | E50SaCas9KKH | AGGCTGCTTTGCCCTCAGC |
| | 1 | GGC | | 6 | TCT |
| 148 | E50SaCas9KKH | GGGATCCAGTATACTTACA | 154 | E50SaCas9KKH | TTTGCCCTCAGCTCTTGAA |
| | 1 | GGC | | 7 | GTA |
| 148 | E50SaCas9KKH | GGGATCCAGTATACTTACA | 155 | E50SaCas9KKH | TTTACCGCCTTCCACTCAG |
| | 1 | GGC | | 8 | AGC |
| 148 | E50SaCas9KKH | GGGATCCAGTATACTTACA | 156 | E50SaCas9KKH | GGACTGACCACTATTGGAG |
| | 1 | GGC | | 10 | CCT |
| 148 | E50SaCas9KKH | GGGATCCAGTATACTTACA | 157 | E50SaCas9KKH | AAGATCTGAGCTCTGAGTG |
| | 1 | GGC | | 11 | GAA |
| 148 | E50SaCas9KKH | GGGATCCAGTATACTTACA | 158 | E50SaCas9KKH | AGGAAGTTAGAAGATCTGA |
| | 1 | GGC | | 12 | GCT |
| 148 | E50SaCas9KKH | GGGATCCAGTATACTTACA | 159 | E50SaCas9KKH | TTTTCTGTTAAAGAGGAAG |
| | 1 | GGC | | 13 | TTA |
| 149 | E50SaCas9KKH | ATCCAGTATACTTACAGGC | 150 | E50SaCas9KKH | CAGTATACTTACAGGCTCC |
| | 2 | TCC | | 3 | AAT |
| 149 | E50SaCas9KKH | ATCCAGTATACTTACAGGC | 151 | E50SaCas9KKH | ATACTTACAGGCTCCAATA |
| | 2 | TCC | | 4 | GTG |
| 149 | E50SaCas9KKH | ATCCAGTATACTTACAGGC | 152 | E50SaCas9KKH | CCAATAGTGGTCAGTCCAG |
| | 2 | TCC | | 5 | GAG |
| 149 | E50SaCas9KKH | ATCCAGTATACTTACAGGC | 153 | E50SaCas9KKH | AGGCTGCTTTGCCCTCAGC |
| | 2 | TCC | | 6 | TCT |
| 149 | E50SaCas9KKH | ATCCAGTATACTTACAGGC | 154 | E50SaCas9KKH | TTTGCCCTCAGCTCTTGAA |
| | 2 | TCC | | 7 | GTA |
| 149 | E50SaCas9KKH | ATCCAGTATACTTACAGGC | 155 | E50SaCas9KKH | TTTACCGCCTTCCACTCAG |
| | 2 | TCC | | 8 | AGC |
| 149 | E50SaCas9KKH | ATCCAGTATACTTACAGGC | 156 | E50SaCas9KKH | GGACTGACCACTATTGGAG |
| | 2 | TCC | | 10 | CCT |
| 149 | E50SaCas9KKH | ATCCAGTATACTTACAGGC | 157 | E50SaCas9KKH | AAGATCTGAGCTCTGAGTG |
| | 2 | TCC | | 11 | GAA |
| 149 | E50SaCas9KKH | ATCCAGTATACTTACAGGC | 158 | E50SaCas9KKH | AGGAAGTTAGAAGATCTGA |
| | 2 | TCC | | 12 | GCT |
| 149 | E50SaCas9KKH | ATCCAGTATACTTACAGGC | 159 | E50SaCas9KKH | TTTTCTGTTAAAGAGGAAG |
| | 2 | TCC | | 13 | TTA |
| 150 | E50SaCas9KKH | CAGTATACTTACAGGCTCC | 151 | E50SaCas9KKH | ATACTTACAGGCTCCAATA |
| | 3 | AAT | | 4 | GTG |
| 150 | E50SaCas9KKH | CAGTATACTTACAGGCTCC | 152 | E50SaCas9KKH | CCAATAGTGGTCAGTCCAG |
| | 3 | AAT | | 5 | GAG |
| 150 | E50SaCas9KKH | CAGTATACTTACAGGCTCC | 153 | E50SaCas9KKH | AGGCTGCTTTGCCCTCAGC |
| | 3 | AAT | | 6 | TCT |
| 150 | E50SaCas9KKH | CAGTATACTTACAGGCTCC | 154 | E50SaCas9KKH | TTTGCCCTCAGCTCTTGAA |
| | 3 | AAT | | 7 | GTA |
| 150 | E50SaCas9KKH | CAGTATACTTACAGGCTCC | 155 | E50SaCas9KKH | TTTACCGCCTTCCACTCAG |
| | 3 | AAT | | 8 | AGC |
| 150 | E50SaCas9KKH | CAGTATACTTACAGGCTCC | 156 | E50SaCas9KKH | GGACTGACCACTATTGGAG |
| | 3 | AAT | | 10 | CCT |
| 150 | E50SaCas9KKH | CAGTATACTTACAGGCTCC | 157 | E50SaCas9KKH | AAGATCTGAGCTCTGAGTG |
| | 3 | AAT | | 11 | GAA |
| 150 | E50SaCas9KKH | CAGTATACTTACAGGCTCC | 158 | E50SaCas9KKH | AGGAAGTTAGAAGATCTGA |
| | 3 | AAT | | 12 | GCT |
| 150 | E50SaCas9KKH | CAGTATACTTACAGGCTCC | 159 | E50SaCas9KKH | TTTTCTGTTAAAGAGGAAG |
| | 3 | AAT | | 13 | TTA |
| 151 | E50SaCas9KKH | ATACTTACAGGCTCCAATA | 152 | E50SaCas9KKH | CCAATAGTGGTCAGTCCAG |
| | 4 | GTG | | 5 | GAG |
| 151 | E50SaCas9KKH | ATACTTACAGGCTCCAATA | 153 | E50SaCas9KKH | AGGCTGCTTTGCCCTCAGC |
| | 4 | GTG | | 6 | TCT |
| 151 | E50SaCas9KKH | ATACTTACAGGCTCCAATA | 154 | E50SaCas9KKH | TTTGCCCTCAGCTCTTGAA |
| | 4 | GTG | | 7 | GTA |
| 151 | E50SaCas9KKH | ATACTTACAGGCTCCAATA | 155 | E50SaCas9KKH | TTTACCGCCTTCCACTCAG |
| | 4 | GTG | | 8 | AGC |
| 151 | E50SaCas9KKH | ATACTTACAGGCTCCAATA | 156 | E50SaCas9KKH | GGACTGACCACTATTGGAG |
| | 4 | GTG | | 10 | CCT |
| 151 | E50SaCas9KKH | ATACTTACAGGCTCCAATA | 157 | E50SaCas9KKH | AAGATCTGAGCTCTGAGTG |
| | 4 | GTG | | 11 | GAA |
| 151 | E50SaCas9KKH | ATACTTACAGGCTCCAATA | 158 | E50SaCas9KKH | AGGAAGTTAGAAGATCTGA |
| | 4 | GTG | | 12 | GCT |
| 151 | E50SaCas9KKH | ATACTTACAGGCTCCAATA | 159 | E50SaCas9KKH | TTTTCTGTTAAAGAGGAAG |
| | 4 | GTG | | 13 | TTA |
| 152 | E50SaCas9KKH | CCAATAGTGGTCAGTCCAG | 153 | E50SaCas9KKH | AGGCTGCTTTGCCCTCAGC |
| | 5 | GAG | | 6 | TCT |
| 152 | E50SaCas9KKH | CCAATAGTGGTCAGTCCAG | 154 | E50SaCas9KKH | TTTGCCCTCAGCTCTTGAA |
| | 5 | GAG | | 7 | GTA |
| 152 | E50SaCas9KKH | CCAATAGTGGTCAGTCCAG | 155 | E50SaCas9KKH | TTTACCGCCTTCCACTCAG |
| | 5 | GAG | | 8 | AGC |
| 152 | E50SaCas9KKH | CCAATAGTGGTCAGTCCAG | 156 | E50SaCas9KKH | GGACTGACCACTATTGGAG |
| | 5 | GAG | | 10 | CCT |
| 152 | E50SaCas9KKH | CCAATAGTGGTCAGTCCAG | 157 | E50SaCas9KKH | AAGATCTGAGCTCTGAGTG |
| | 5 | GAG | | 11 | GAA |
| 152 | E50SaCas9KKH | CCAATAGTGGTCAGTCCAG | 158 | E50SaCas9KKH | AGGAAGTTAGAAGATCTGA |
| | 5 | GAG | | 12 | GCT |
| 152 | E50SaCas9KKH | CCAATAGTGGTCAGTCCAG | 159 | E50SaCas9KKH | TTTTCTGTTAAAGAGGAAG |
| | 5 | GAG | | 13 | TTA |
| 153 | E50SaCas9KKH | AGGCTGCTTTGCCCTCAGC | 154 | E50SaCas9KKH | TTTGCCCTCAGCTCTTGAA |
| | 6 | TCT | | 7 | GTA |
| 153 | E50SaCas9KKH | AGGCTGCTTTGCCCTCAGC | 155 | E50SaCas9KKH | TTTACCGCCTTCCACTCAG |
| | 6 | TCT | | 8 | AGC |
| 153 | E50SaCas9KKH | AGGCTGCTTTGCCCTCAGC | 156 | E50SaCas9KKH | GGACTGACCACTATTGGAG |
| | 6 | TCT | | 10 | CCT |
| 153 | E50SaCas9KKH | AGGCTGCTTTGCCCTCAGC | 157 | E50SaCas9KKH | AAGATCTGAGCTCTGAGTG |
| | 6 | TCT | | 11 | GAA |
| 153 | E50SaCas9KKH | AGGCTGCTTTGCCCTCAGC | 158 | E50SaCas9KKH | AGGAAGTTAGAAGATCTGA |
| | 6 | TCT | | 12 | GCT |
| 153 | E50SaCas9KKH | AGGCTGCTTTGCCCTCAGC | 159 | E50SaCas9KKH | TTTTCTGTTAAAGAGGAAG |
| | 6 | TCT | | 13 | TTA |
| 154 | E50SaCas9KKH | TTTGCCCTCAGCTCTTGAA | 155 | E50SaCas9KKH | TTTACCGCCTTCCACTCAG |
| | 7 | GTA | | 8 | AGC |
| 154 | E50SaCas9KKH | TTTGCCCTCAGCTCTTGAA | 156 | E50SaCas9KKH | GGACTGACCACTATTGGAG |
| | 7 | GTA | | 10 | CCT |
| 154 | E50SaCas9KKH | TTTGCCCTCAGCTCTTGAA | 157 | E50SaCas9KKH | AAGATCTGAGCTCTGAGTG |
| | 7 | GTA | | 11 | GAA |
| 154 | E50SaCas9KKH | TTTGCCCTCAGCTCTTGAA | 158 | E50SaCas9KKH | AGGAAGTTAGAAGATCTGA |
| | 7 | GTA | | 12 | GCT |
| 154 | E50SaCas9KKH | TTTGCCCTCAGCTCTTGAA | 159 | E50SaCas9KKH | TTTTCTGTTAAAGAGGAAG |
| | 7 | GTA | | 13 | TTA |
| 155 | E50SaCas9KKH | TTTACCGCCTTCCACTCAG | 156 | E50SaCas9KKH | GGACTGACCACTATTGGAG |
| | 8 | AGC | | 10 | CCT |
| 155 | E50SaCas9KKH | TTTACCGCCTTCCACTCAG | 157 | E50SaCas9KKH | AAGATCTGAGCTCTGAGTG |
| | 8 | AGC | | 11 | GAA |
| 155 | E50SaCas9KKH | TTTACCGCCTTCCACTCAG | 158 | E50SaCas9KKH | AGGAAGTTAGAAGATCTGA |
| | 8 | AGC | | 12 | GCT |
| 155 | E50SaCas9KKH | TTTACCGCCTTCCACTCAG | 159 | E50SaCas9KKH | TTTTCTGTTAAAGAGGAAG |
| | 8 | AGC | | 13 | TTA |
| 156 | E50SaCas9KKH | GGACTGACCACTATTGGAG | 157 | E50SaCas9KKH | AAGATCTGAGCTCTGAGTG |
| | 10 | CCT | | 11 | GAA |
| 156 | E50SaCas9KKH | GGACTGACCACTATTGGAG | 158 | E50SaCas9KKH | AGGAAGTTAGAAGATCTGA |
| | 10 | CCT | | 12 | GCT |
| 156 | E50SaCas9KKH | GGACTGACCACTATTGGAG | 159 | E50SaCas9KKH | TTTTCTGTTAAAGAGGAAG |
| | 10 | CCT | | 13 | TTA |
| 157 | E50SaCas9KKH | AAGATCTGAGCTCTGAGTG | 158 | E50SaCas9KKH | AGGAAGTTAGAAGATCTGA |
| | 11 | GAA | | 12 | GCT |
| 157 | E50SaCas9KKH | AAGATCTGAGCTCTGAGTG | 159 | E50SaCas9KKH | TTTTCTGTTAAAGAGGAAG |
| | 11 | GAA | | 13 | TTA |
| 158 | E50SaCas9KKH | AGGAAGTTAGAAGATCTGA | 159 | E50SaCas9KKH | TTTTCTGTTAAAGAGGAAG |
| | 12 | GCT | | 13 | TTA |

TABLE 3C
(3) Exon 50 SluCas9 guides:

| Seq | Guide ID or | |
|---|---|---|
| ID No. | Guide RNA Name | Sequence |
| 231 | E50Slu2 | AGTATACTTACAGGCTCCAATA |
| 232 | E50Slu3 | CAGGCTCCAATAGTGGTCAGTC |
| 233 | E50Slu4 | CAATAGTGGTCAGTCCAGGAGC |
| 234 | E50Slu5 | GTGGTCAGTCCAGGAGCTAGGT |
| 235 | E50Slu6 | TTGCCCTCAGCTCTTGAAGTAA |
| 236 | E50Slu9 | CTAGCTCCTGGACTGACCACTA |
| 237 | E50Slu10 | GCAAAGCAGCCTGACCTAGCTC |
| 238 | E50Slu11 | AAACCGTTTACTTCAAGAGCTG |
| 239 | E50Slu12 | TAAACCGTTTACTTCAAGAGCT |
| 240 | E50Slu13 | AGATCTGAGCTCTGAGTGGAAG |
| 241 | E50Slu14 | AGAAGATCTGAGCTCTGAGTGG |
| 242 | E50Slu15 | AGTTAGAAGATCTGAGCTCTGA |

TABLE 3D
(4) Exon 50 SluCas9 pairs:

| Guide 1 | | | Guide 2 | | |
|---|---|---|---|---|---|
| Seq ID No. | Guide 1 | Guide 1 Sequence | Seq ID No. | Guide 2 | Guide 2 Sequence |
| 231 | E50SL2 | AGTATACTTACAGGCTCCAATA | 232 | E50SL3 | CAGGCTCCAATAGTGGTCAGTC |
| 231 | E50SL2 | AGTATACTTACAGGCTCCAATA | 233 | E50SL4 | CAATAGTGGTCAGTCCAGGAGC |
| 231 | E50SL2 | AGTATACTTACAGGCTCCAATA | 234 | E50SL5 | GTGGTCAGTCCAGGAGCTAGGT |
| 231 | E50SL2 | AGTATACTTACAGGCTCCAATA | 235 | E50SL6 | TTGCCCTCAGCTCTTGAAGTAA |
| 231 | E50SL2 | AGTATACTTACAGGCTCCAATA | 236 | E50SL9 | CTAGCTCCTGGACTGACCACTA |
| 231 | E50SL2 | AGTATACTTACAGGCTCCAATA | 237 | E50SL10 | GCAAAGCAGCCTGACCTAGCTC |
| 231 | E50SL2 | AGTATACTTACAGGCTCCAATA | 238 | E50SL11 | AAACCGTTTACTTCAAGAGCTG |
| 231 | E50SL2 | AGTATACTTACAGGCTCCAATA | 239 | E50SL12 | TAAACCGTTTACTTCAAGAGCT |
| 231 | E50SL2 | AGTATACTTACAGGCTCCAATA | 240 | E50SL13 | AGATCTGAGCTCTGAGTGGAAG |
| 231 | E50SL2 | AGTATACTTACAGGCTCCAATA | 241 | E50SL14 | AGAAGATCTGAGCTCTGAGTGG |
| 231 | E50SL2 | AGTATACTTACAGGCTCCAATA | 242 | E50SL15 | AGTTAGAAGATCTGAGCTCTGA |
| 232 | E50SL3 | CAGGCTCCAATAGTGGTCAGTC | 233 | E50SL4 | CAATAGTGGTCAGTCCAGGAGC |
| 232 | E50SL3 | CAGGCTCCAATAGTGGTCAGTC | 234 | E50SL5 | GTGGTCAGTCCAGGAGCTAGGT |
| 232 | E50SL3 | CAGGCTCCAATAGTGGTCAGTC | 235 | E50SL6 | TTGCCCTCAGCTCTTGAAGTAA |
| 232 | E50SL3 | CAGGCTCCAATAGTGGTCAGTC | 236 | E50SL9 | CTAGCTCCTGGACTGACCACTA |
| 232 | E50SL3 | CAGGCTCCAATAGTGGTCAGTC | 237 | E50SL10 | GCAAAGCAGCCTGACCTAGCTC |
| 232 | E50SL3 | CAGGCTCCAATAGTGGTCAGTC | 238 | E50SL11 | AAACCGTTTACTTCAAGAGCTG |
| 232 | E50SL3 | CAGGCTCCAATAGTGGTCAGTC | 239 | E50SL12 | TAAACCGTTTACTTCAAGAGCT |
| 232 | E50SL3 | CAGGCTCCAATAGTGGTCAGTC | 240 | E50SL13 | AGATCTGAGCTCTGAGTGGAAG |
| 232 | E50SL3 | CAGGCTCCAATAGTGGTCAGTC | 241 | E50SL14 | AGAAGATCTGAGCTCTGAGTGG |
| 232 | E50SL3 | CAGGCTCCAATAGTGGTCAGTC | 242 | E50SL15 | AGTTAGAAGATCTGAGCTCTGA |
| 233 | E50SL4 | CAATAGTGGTCAGTCCAGGAGC | 234 | E50SL5 | GTGGTCAGTCCAGGAGCTAGGT |
| 233 | E50SL4 | CAATAGTGGTCAGTCCAGGAGC | 235 | E50SL6 | TTGCCCTCAGCTCTTGAAGTAA |
| 233 | E50SL4 | CAATAGTGGTCAGTCCAGGAGC | 236 | E50SL9 | CTAGCTCCTGGACTGACCACTA |
| 233 | E50SL4 | CAATAGTGGTCAGTCCAGGAGC | 237 | E50SL10 | GCAAAGCAGCCTGACCTAGCTC |
| 233 | E50SL4 | CAATAGTGGTCAGTCCAGGAGC | 238 | E50SL11 | AAACCGTTTACTTCAAGAGCTG |
| 233 | E50SL4 | CAATAGTGGTCAGTCCAGGAGC | 239 | E50SL12 | TAAACCGTTTACTTCAAGAGCT |
| 233 | E50SL4 | CAATAGTGGTCAGTCCAGGAGC | 240 | E50SL13 | AGATCTGAGCTCTGAGTGGAAG |
| 233 | E50SL4 | CAATAGTGGTCAGTCCAGGAGC | 241 | E50SL14 | AGAAGATCTGAGCTCTGAGTGG |
| 233 | E50SL4 | CAATAGTGGTCAGTCCAGGAGC | 242 | E50SL15 | AGTTAGAAGATCTGAGCTCTGA |
| 234 | E50SL5 | GTGGTCAGTCCAGGAGCTAGGT | 235 | E50SL6 | TTGCCCTCAGCTCTTGAAGTAA |
| 234 | E50SL5 | GTGGTCAGTCCAGGAGCTAGGT | 236 | E50SL9 | CTAGCTCCTGGACTGACCACTA |
| 234 | E50SL5 | GTGGTCAGTCCAGGAGCTAGGT | 237 | E50SL10 | GCAAAGCAGCCTGACCTAGCTC |
| 234 | E50SL5 | GTGGTCAGTCCAGGAGCTAGGT | 238 | E50SL11 | AAACCGTTTACTTCAAGAGCTG |
| 234 | E50SL5 | GTGGTCAGTCCAGGAGCTAGGT | 239 | E50SL12 | TAAACCGTTTACTTCAAGAGCT |
| 234 | E50SL5 | GTGGTCAGTCCAGGAGCTAGGT | 240 | E50SL13 | AGATCTGAGCTCTGAGTGGAAG |
| 234 | E50SL5 | GTGGTCAGTCCAGGAGCTAGGT | 241 | E50SL14 | AGAAGATCTGAGCTCTGAGTGG |
| 234 | E50SL5 | GTGGTCAGTCCAGGAGCTAGGT | 242 | E50SL15 | AGTTAGAAGATCTGAGCTCTGA |
| 235 | E50SL6 | TTGCCCTCAGCTCTTGAAGTAA | 236 | E50SL9 | CTAGCTCCTGGACTGACCACTA |
| 235 | E50SL6 | TTGCCCTCAGCTCTTGAAGTAA | 237 | E50SL10 | GCAAAGCAGCCTGACCTAGCTC |
| 235 | E50SL6 | TTGCCCTCAGCTCTTGAAGTAA | 238 | E50SL11 | AAACCGTTTACTTCAAGAGCTG |
| 235 | E50SL6 | TTGCCCTCAGCTCTTGAAGTAA | 239 | E50SL12 | TAAACCGTTTACTTCAAGAGCT |
| 235 | E50SL6 | TTGCCCTCAGCTCTTGAAGTAA | 240 | E50SL13 | AGATCTGAGCTCTGAGTGGAAG |
| 235 | E50SL6 | TTGCCCTCAGCTCTTGAAGTAA | 241 | E50SL14 | AGAAGATCTGAGCTCTGAGTGG |
| 235 | E50SL6 | TTGCCCTCAGCTCTTGAAGTAA | 242 | E50SL15 | AGTTAGAAGATCTGAGCTCTGA |
| 236 | E50SL9 | CTAGCTCCTGGACTGACCACTA | 237 | E50SL10 | GCAAAGCAGCCTGACCTAGCTC |
| 236 | E50SL9 | CTAGCTCCTGGACTGACCACTA | 238 | E50SL11 | AAACCGTTTACTTCAAGAGCTG |
| 236 | E50SL9 | CTAGCTCCTGGACTGACCACTA | 239 | E50SL12 | TAAACCGTTTACTTCAAGAGCT |
| 236 | E50SL9 | CTAGCTCCTGGACTGACCACTA | 240 | E50SL13 | AGATCTGAGCTCTGAGTGGAAG |
| 236 | E50SL9 | CTAGCTCCTGGACTGACCACTA | 241 | E50SL14 | AGAAGATCTGAGCTCTGAGTGG |
| 236 | E50SL9 | CTAGCTCCTGGACTGACCACTA | 242 | E50SL15 | AGTTAGAAGATCTGAGCTCTGA |
| 237 | E50SL10 | GCAAAGCAGCCTGACCTAGCTC | 238 | E50SL11 | AAACCGTTTACTTCAAGAGCTG |
| 237 | E50SL10 | GCAAAGCAGCCTGACCTAGCTC | 239 | E50SL12 | TAAACCGTTTACTTCAAGAGCT |
| 237 | E50SL10 | GCAAAGCAGCCTGACCTAGCTC | 240 | E50SL13 | AGATCTGAGCTCTGAGTGGAAG |
| 237 | E50SL10 | GCAAAGCAGCCTGACCTAGCTC | 241 | E50SL14 | AGAAGATCTGAGCTCTGAGTGG |
| 237 | E50SL10 | GCAAAGCAGCCTGACCTAGCTC | 242 | E50SL15 | AGTTAGAAGATCTGAGCTCTGA |
| 238 | E50SL11 | AAACCGTTTACTTCAAGAGCTG | 239 | E50SL12 | TAAACCGTTTACTTCAAGAGCT |
| 238 | E50SL11 | AAACCGTTTACTTCAAGAGCTG | 240 | E50SL13 | AGATCTGAGCTCTGAGTGGAAG |
| 238 | E50SL11 | AAACCGTTTACTTCAAGAGCTG | 241 | E50SL14 | AGAAGATCTGAGCTCTGAGTGG |
| 238 | E50SL11 | AAACCGTTTACTTCAAGAGCTG | 242 | E50SL15 | AGTTAGAAGATCTGAGCTCTGA |
| 239 | E50SL12 | TAAACCGTTTACTTCAAGAGCT | 240 | E50SL13 | AGATCTGAGCTCTGAGTGGAAG |
| 239 | E50SL12 | TAAACCGTTTACTTCAAGAGCT | 241 | E50SL14 | AGAAGATCTGAGCTCTGAGTGG |
| 239 | E50SL12 | TAAACCGTTTACTTCAAGAGCT | 242 | E50SL15 | AGTTAGAAGATCTGAGCTCTGA |
| 240 | E50SL13 | AGATCTGAGCTCTGAGTGGAAG | 241 | E50SL14 | AGAAGATCTGAGCTCTGAGTGG |
| 240 | E50SL13 | AGATCTGAGCTCTGAGTGGAAG | 242 | E50SL15 | AGTTAGAAGATCTGAGCTCTGA |
| 241 | E50SL14 | AGAAGATCTGAGCTCTGAGTGG | 242 | E50SL15 | AGTTAGAAGATCTGAGCTCTGA |

### d) Exon 51:

TABLE 4A
(1) Exon 51 SaCas9 guides:

| Seq | Guide ID or | |
|---|---|---|
| ID No. | Guide RNA Name | Guide Sequence |
| 11 | E51Sa1 | TAGTAACCACAGGTTGTGTCAC |
| 12 | E51Sa2 | GTTGTGTCACCAGAGTAACAGT |
| 13 | E51Sa4 | GAGGGTGATGGTGGGTGACCTT |
| 14 | E51Sa5 | TATAAAATCACAGAGGGTGATG |
| 15 | E51Sa6 | TTGATCAAGTTATAAAATCACA |
| 27 | E51SaCas9KKH1 | TCATTTTTTCTCATACCTTCTG |
| 28 | E51SaCas9KKH2 | TTTTTTCTCATACCTTCTGCTT |
| 29 | E51SaCas9KKH3 | CCTTCTGCTTGATGATCATCTC |
| 30 | E51SaCas9KKH4 | ATGATCATCTCGTTGATATCCT |
| 31 | E51SaCas9KKH5 | AAGGTCACCCACCATCACCCTC |
| 32 | E51SaCas9KKH6 | ATCACCCTCTGTGATTTTATAA |
| 33 | E51SaCas9KKH7 | ATAACTTGATCAAGCAGAGAAA |
| 34 | E51SaCas9KKH8 | CTTGATCAAGCAGAGAAAGCCA |
| 35 | E51SaCas9KKH9 | ATCAAGCAGAGAAAGCCAGTCG |
| 36 | E51SaCas9KKH10 | CAGTCGGTAAGTTCTGTCCAAG |
| 37 | E51SaCas9KKH11 | GTAAGTTCTGTCCAAGCCCGGT |
| 38 | E51SaCas9KKH12 | AGCCCGGTTGAAATCTGCCAGA |
| 39 | E51SaCas9KKH13 | AGCAGGTACCTCCAACATCAAG |
| 40 | E51SaCas9KKH14 | CAACATCAAGGAAGATGGCATT |
| 41 | E51SaCas9KKH15 | AGGAAGATGGCATTTCTAGTTT |
| 42 | E51SaCas9KKH16 | ATGGCATTTCTAGTTTGGAGAT |
| 43 | E51SaCas9KKH17 | CTAGTTTGGAGATGGCAGTTTC |
| 44 | E51SaCas9KKH18 | GATGGCAGTTTCCTTAGTAACC |
| 45 | E51SaCas9KKH19 | TAGTAACCACAGGTTGTGTCAC |
| 46 | E51SaCas9KKH20 | CCACAGGTTGTGTCACCAGAGT |
| 47 | E51SaCas9KKH21 | GTTGTGTCACCAGAGTAACAGT |
| 48 | E51SaCas9KKH22 | GAGTAACAGTCTGAGTAGGAGC |
| 49 | E51SaCas9KKH23 | TCTGAGTAGGAGCTAAAATATT |
| 50 | E51SaCas9KKH24 | AGAAGGTATGAGAAAAAATGAT |
| 51 | E51SaCas9KKH25 | TCAAGCAGAAGGTATGAGAAAA |
| 52 | E51SaCas9KKH26 | TCATCAAGCAGAAGGTATGAGA |
| 53 | E51SaCas9KKH27 | TCAACGAGATGATCATCAAGCA |
| 54 | E51SaCas9KKH28 | GTGACCTTGAGGATATCAACGA |
| 55 | E51SaCas9KKH29 | TGGGTGACCTTGAGGATATCAA |
| 56 | E51SaCas9KKH30 | GAGGGTGATGGTGGGTGACCTT |
| 57 | E51SaCas9KKH31 | TATAAAATCACAGAGGGTGATG |
| 58 | E51SaCas9KKH32 | AAGTTATAAAATCACAGAGGGT |
| 59 | E51SaCas9KKH33 | ATCAAGTTATAAAATCACAGAG |
| 60 | E51SaCas9KKH34 | TTGATCAAGTTATAAAATCACA |
| 61 | E51SaCas9KKH35 | CTTTCTCTGCTTGATCAAGTTA |
| 62 | E51SaCas9KKH36 | CCGACTGGCTTTCTCTGCTTGA |
| 63 | E51SaCas9KKH37 | ACTTACCGACTGGCTTTCTCTG |
| 64 | E51SaCas9KKH38 | GATGTTGGAGGTACCTGCTCTG |
| 65 | E51SaCas9KKH39 | AAATGCCATCTTCCTTGATGTT |
| 66 | E51SaCas9KKH40 | CCAAACTAGAAATGCCATCTTC |
| 67 | E51SaCas9KKH41 | AGGAAACTGCCATCTCCAAACT |
| 68 | E51SaCas9KKH42 | CTGTTACTCTGGTGACACAACC |
| 69 | E51SaCas9KKH43 | TAGCTCCTACTCAGACTGTTAC |

TABLE 4B
(2) Exon 51 SaCas9 pairs:

| Guide 1 | | | Guide 2 | | |
|---|---|---|---|---|---|
| Seq ID No. | Guide 1 | Guide 1 Sequence | Seq ID No. | Guide 2 | Guide 2 Sequence |
| 11 | E51Sa1 | TAGTAACCACAGGTTGTGT | 12 | E51Sa2 | GTTGTGTCACCAGAGTAAC |
| | | CAC | | | AGT |
| 11 | E51Sa1 | TAGTAACCACAGGTTGTGT | 13 | E51Sa4 | GAGGGTGATGGTGGGTGAC |
| | | CAC | | | CTT |
| 11 | E51Sa1 | TAGTAACCACAGGTTGTGT | 14 | E51Sa5 | TATAAAATCACAGAGGGTG |
| | | CAC | | | ATG |
| 11 | E51Sa1 | TAGTAACCACAGGTTGTGT | 15 | E51Sa6 | TTGATCAAGTTATAAAATC |
| | | CAC | | | ACA |
| 12 | E51Sa2 | GTTGTGTCACCAGAGTAAC | 13 | E51Sa4 | GAGGGTGATGGTGGGTGAC |
| | | AGT | | | CTT |
| 12 | E51Sa2 | GTTGTGTCACCAGAGTAAC | 14 | E51Sa5 | TATAAAATCACAGAGGGTG |
| | | AGT | | | ATG |
| 12 | E51Sa2 | GTTGTGTCACCAGAGTAAC | 15 | E51Sa6 | TTGATCAAGTTATAAAATC |
| | | AGT | | | ACA |
| 13 | E51Sa4 | GAGGGTGATGGTGGGTGAC | 14 | E51Sa5 | TATAAAATCACAGAGGGTG |
| | | CTT | | | ATG |
| 13 | E51Sa4 | GAGGGTGATGGTGGGTGAC | 15 | E51Sa6 | TTGATCAAGTTATAAAATC |
| | | CTT | | | ACA |
| 14 | E51Sa5 | TATAAAATCACAGAGGGTG | 15 | E51Sa6 | TTGATCAAGTTATAAAATC |
| | | ATG | | | ACA |
| 27 | E51SaCas9KKH | TCATTTTTTCTCATACCTT | 28 | E51SaCas9KKH | TTTTTTCTCATACCTTCTG |
| | 1 | CTG | | 2 | CTT |
| 27 | E51SaCas9KKH | TCATTTTTTCTCATACCTT | 29 | E51SaCas9KKH | CCTTCTGCTTGATGATCAT |
| | 1 | CTG | | 3 | CTC |
| 27 | E51SaCas9KKH | TCATTTTTTCTCATACCTT | 30 | E51SaCas9KKH | ATGATCATCTCGTTGATAT |
| | 1 | CTG | | 4 | CCT |
| 27 | E51SaCas9KKH | TCATTTTTTCTCATACCTT | 31 | E51SaCas9KKH | AAGGTCACCCACCATCACC |
| | 1 | CTG | | 5 | CTC |
| 27 | E51SaCas9KKH | TCATTTTTTCTCATACCTT | 32 | E51SaCas9KKH | ATCACCCTCTGTGATTTTA |
| | 1 | CTG | | 6 | TAA |
| 27 | E51SaCas9KKH | TCATTTTTTCTCATACCTT | 33 | E51SaCas9KKH | ATAACTTGATCAAGCAGAG |
| | 1 | CTG | | 7 | AAA |
| 27 | E51SaCas9KKH | TCATTTTTTCTCATACCTT | 34 | E51SaCas9KKH | CTTGATCAAGCAGAGAAAG |
| | 1 | CTG | | 8 | CCA |
| 27 | E51SaCas9KKH | TCATTTTTTCTCATACCTT | 35 | E51SaCas9KKH | ATCAAGCAGAGAAAGCCAG |
| | 1 | CTG | | 9 | TCG |
| 27 | E51SaCas9KKH | TCATTTTTTCTCATACCTT | 36 | E51SaCas9KKH | CAGTCGGTAAGTTCTGTCC |
| | 1 | CTG | | 10 | AAG |
| 27 | E51SaCas9KKH | TCATTTTTTCTCATACCTT | 37 | E51SaCas9KKH | GTAAGTTCTGTCCAAGCCC |
| | 1 | CTG | | 11 | GGT |
| 27 | E51SaCas9KKH | TCATTTTTTCTCATACCTT | 38 | E51SaCas9KKH | AGCCCGGTTGAAATCTGCC |
| | 1 | CTG | | 12 | AGA |
| 27 | E51SaCas9KKH | TCATTTTTTCTCATACCTT | 39 | E51SaCas9KKH | AGCAGGTACCTCCAACATC |
| | 1 | CTG | | 13 | AAG |
| 27 | E51SaCas9KKH | TCATTTTTTCTCATACCTT | 40 | E51SaCas9KKH | CAACATCAAGGAAGATGGC |
| | 1 | CTG | | 14 | ATT |
| 27 | E51SaCas9KKH | TCATTTTTTCTCATACCTT | 41 | E51SaCas9KKH | AGGAAGATGGCATTTCTAG |
| | 1 | CTG | | 15 | TTT |
| 27 | E51SaCas9KKH | TCATTTTTTCTCATACCTT | 42 | E51SaCas9KKH | ATGGCATTTCTAGTTTGGA |
| | 1 | CTG | | 16 | GAT |
| 27 | E51SaCas9KKH | TCATTTTTTCTCATACCTT | 43 | E51SaCas9KKH | CTAGTTTGGAGATGGCAGT |
| | 1 | CTG | | 17 | TTC |
| 27 | E51SaCas9KKH | TCATTTTTTCTCATACCTT | 44 | E51SaCas9KKH | GATGGCAGTTTCCTTAGTA |
| | 1 | CTG | | 18 | ACC |
| 27 | E51SaCas9KKH | TCATTTTTTCTCATACCTT | 45 | E51SaCas9KKH | TAGTAACCACAGGTTGTGT |
| | 1 | CTG | | 19 | CAC |
| 27 | E51SaCas9KKH | TCATTTTTTCTCATACCTT | 46 | E51SaCas9KKH | CCACAGGTTGTGTCACCAG |
| | 1 | CTG | | 20 | AGT |
| 27 | E51SaCas9KKH | TCATTTTTTCTCATACCTT | 47 | E51SaCas9KKH | GTTGTGTCACCAGAGTAAC |
| | 1 | CTG | | 21 | AGT |
| 27 | E51SaCas9KKH | TCATTTTTTCTCATACCTT | 48 | E51SaCas9KKH | GAGTAACAGTCTGAGTAGG |
| | 1 | CTG | | 22 | AGC |
| 27 | E51SaCas9KKH | TCATTTTTTCTCATACCTT | 53 | E51SaCas9KKH | TCAACGAGATGATCATCAA |
| | 1 | CTG | | 27 | GCA |
| 27 | E51SaCas9KKH | TCATTTTTTCTCATACCTT | 54 | E51SaCas9KKH | GTGACCTTGAGGATATCAA |
| | 1 | CTG | | 28 | CGA |
| 27 | E51SaCas9KKH | TCATTTTTTCTCATACCTT | 55 | E51SaCas9KKH | TGGGTGACCTTGAGGATAT |
| | 1 | CTG | | 29 | CAA |
| 27 | E51SaCas9KKH | TCATTTTTTCTCATACCTT | 56 | E51SaCas9KKH | GAGGGTGATGGTGGGTGAC |
| | 1 | CTG | | 30 | CTT |
| 27 | E51SaCas9KKH | TCATTTTTTCTCATACCTT | 57 | E51SaCas9KKH | TATAAAATCACAGAGGGTG |
| | 1 | CTG | | 31 | ATG |
| 27 | E51SaCas9KKH | TCATTTTTTCTCATACCTT | 58 | E51SaCas9KKH | AAGTTATAAAATCACAGAG |
| | 1 | CTG | | 32 | GGT |
| 27 | E51SaCas9KKH | TCATTTTTTCTCATACCTT | 59 | E51SaCas9KKH | ATCAAGTTATAAAATCACA |
| | 1 | CTG | | 33 | GAG |
| 27 | E51SaCas9KKH | TCATTTTTTCTCATACCTT | 60 | E51SaCas9KKH | TTGATCAAGTTATAAAATC |
| | 1 | CTG | | 34 | ACA |
| 27 | E51SaCas9KKH | TCATTTTTTCTCATACCTT | 61 | E51SaCas9KKH | CTTTCTCTGCTTGATCAAG |
| | 1 | CTG | | 35 | TTA |
| 27 | E51SaCas9KKH | TCATTTTTTCTCATACCTT | 62 | E51SaCas9KKH | CCGACTGGCTTTCTCTGCT |
| | 1 | CTG | | 36 | TGA |
| 27 | E51SaCas9KKH | TCATTTTTTCTCATACCTT | 63 | E51SaCas9KKH | ACTTACCGACTGGCTTTCT |
| | 1 | CTG | | 37 | CTG |
| 27 | E51SaCas9KKH | TCATTTTTTCTCATACCTT | 64 | E51SaCas9KKH | GATGTTGGAGGTACCTGCT |
| | 1 | CTG | | 38 | CTG |
| 27 | E51SaCas9KKH | TCATTTTTTCTCATACCTT | 65 | E51SaCas9KKH | AAATGCCATCTTCCTTGAT |
| | 1 | CTG | | 39 | GTT |
| 27 | E51SaCas9KKH | TCATTTTTTCTCATACCTT | 66 | E51SaCas9KKH | CCAAACTAGAAATGCCATC |
| | 1 | CTG | | 40 | TTC |
| 27 | E51SaCas9KKH | TCATTTTTTCTCATACCTT | 67 | E51SaCas9KKH | AGGAAACTGCCATCTCCAA |
| | 1 | CTG | | 41 | ACT |
| 27 | E51SaCas9KKH | TCATTTTTTCTCATACCTT | 68 | E51SaCas9KKH | CTGTTACTCTGGTGACACA |
| | 1 | CTG | | 42 | ACC |
| 27 | E51SaCas9KKH | TCATTTTTTCTCATACCTT | 69 | E51SaCas9KKH | TAGCTCCTACTCAGACTGT |
| | 1 | CTG | | 43 | TAC |
| 28 | E51SaCas9KKH | TTTTTTCTCATACCTTCTG | 29 | E51SaCas9KKH | CCTTCTGCTTGATGATCAT |
| | 2 | CTT | | 3 | CTC |
| 28 | E51SaCas9KKH | TTTTTTCTCATACCTTCTG | 30 | E51SaCas9KKH | ATGATCATCTCGTTGATAT |
| | 2 | CTT | | 4 | CCT |
| 28 | E51SaCas9KKH | TTTTTTCTCATACCTTCTG | 31 | E51SaCas9KKH | AAGGTCACCCACCATCACC |
| | 2 | CTT | | 5 | CTC |
| 28 | E51SaCas9KKH | TTTTTTCTCATACCTTCTG | 32 | E51SaCas9KKH | ATCACCCTCTGTGATTTTA |
| | 2 | CTT | | 6 | TAA |
| 28 | E51SaCas9KKH | TTTTTTCTCATACCTTCTG | 33 | E51SaCas9KKH | ATAACTTGATCAAGCAGAG |
| | 2 | CTT | | 7 | AAA |
| 28 | E51SaCas9KKH | TTTTTTCTCATACCTTCTG | 34 | E51SaCas9KKH | CTTGATCAAGCAGAGAAAG |
| | 2 | CTT | | 8 | CCA |
| 28 | E51SaCas9KKH | TTTTTTCTCATACCTTCTG | 35 | E51SaCas9KKH | ATCAAGCAGAGAAAGCCAG |
| | 2 | CTT | | 9 | TCG |
| 28 | E51SaCas9KKH | TTTTTTCTCATACCTTCTG | 36 | E51SaCas9KKH | CAGTCGGTAAGTTCTGTCC |
| | 2 | CTT | | 10 | AAG |
| 28 | E51SaCas9KKH | TTTTTTCTCATACCTTCTG | 37 | E51SaCas9KKH | GTAAGTTCTGTCCAAGCCC |
| | 2 | CTT | | 11 | GGT |
| 28 | E51SaCas9KKH | TTTTTTCTCATACCTTCTG | 38 | E51SaCas9KKH | AGCCCGGTTGAAATCTGCC |
| | 2 | CTT | | 12 | AGA |
| 28 | E51SaCas9KKH | TTTTTTCTCATACCTTCTG | 39 | E51SaCas9KKH | AGCAGGTACCTCCAACATC |
| | 2 | CTT | | 13 | AAG |
| 28 | E51SaCas9KKH | TTTTTTCTCATACCTTCTG | 40 | E51SaCas9KKH | CAACATCAAGGAAGATGGC |
| | 2 | CTT | | 14 | ATT |
| 28 | E51SaCas9KKH | TTTTTTCTCATACCTTCTG | 41 | E51SaCas9KKH | AGGAAGATGGCATTTCTAG |
| | 2 | CTT | | 15 | TTT |
| 28 | E51SaCas9KKH | TTTTTTCTCATACCTTCTG | 42 | E51SaCas9KKH | ATGGCATTTCTAGTTTGGA |
| | 2 | CTT | | 16 | GAT |
| 28 | E51SaCas9KKH | TTTTTTCTCATACCTTCTG | 43 | E51SaCas9KKH | CTAGTTTGGAGATGGCAGT |
| | 2 | CTT | | 17 | TTC |
| 28 | E51SaCas9KKH | TTTTTTCTCATACCTTCTG | 44 | E51SaCas9KKH | GATGGCAGTTTCCTTAGTA |
| | 2 | CTT | | 18 | ACC |
| 28 | E51SaCas9KKH | TTTTTTCTCATACCTTCTG | 45 | E51SaCas9KKH | TAGTAACCACAGGTTGTGT |
| | 2 | CTT | | 19 | CAC |
| 28 | E51SaCas9KKH | TTTTTTCTCATACCTTCTG | 46 | E51SaCas9KKH | CCACAGGTTGTGTCACCAG |
| | 2 | CTT | | 20 | AGT |
| 28 | E51SaCas9KKH | TTTTTTCTCATACCTTCTG | 47 | E51SaCas9KKH | GTTGTGTCACCAGAGTAAC |
| | 2 | CTT | | 21 | AGT |
| 28 | E51SaCas9KKH | TTTTTTCTCATACCTTCTG | 48 | E51SaCas9KKH | GAGTAACAGTCTGAGTAGG |
| | 2 | CTT | | 22 | AGC |
| 28 | E51SaCas9KKH | TTTTTTCTCATACCTTCTG | 53 | E51SaCas9KKH | TCAACGAGATGATCATCAA |
| | 2 | CTT | | 27 | GCA |
| 28 | E51SaCas9KKH | TTTTTTCTCATACCTTCTG | 54 | E51SaCas9KKH | GTGACCTTGAGGATATCAA |
| | 2 | CTT | | 28 | CGA |
| 28 | E51SaCas9KKH | TTTTTTCTCATACCTTCTG | 55 | E51SaCas9KKH | TGGGTGACCTTGAGGATAT |
| | 2 | CTT | | 29 | CAA |
| 28 | E51SaCas9KKH | TTTTTTCTCATACCTTCTG | 56 | E51SaCas9KKH | GAGGGTGATGGTGGGTGAC |
| | 2 | CTT | | 30 | CTT |
| 28 | E51SaCas9KKH | TTTTTTCTCATACCTTCTG | 57 | E51SaCas9KKH | TATAAAATCACAGAGGGTG |
| | 2 | CTT | | 31 | ATG |
| 28 | E51SaCas9KKH | TTTTTTCTCATACCTTCTG | 58 | E51SaCas9KKH | AAGTTATAAAATCACAGAG |
| | 2 | CTT | | 32 | GGT |
| 28 | E51SaCas9KKH | TTTTTTCTCATACCTTCTG | 59 | E51SaCas9KKH | ATCAAGTTATAAAATCACA |
| | 2 | CTT | | 33 | GAG |
| 28 | E51SaCas9KKH | TTTTTTCTCATACCTTCTG | 60 | E51SaCas9KKH | TTGATCAAGTTATAAAATC |
| | 2 | CTT | | 34 | ACA |
| 28 | E51SaCas9KKH | TTTTTTCTCATACCTTCTG | 61 | E51SaCas9KKH | CTTTCTCTGCTTGATCAAG |
| | 2 | CTT | | 35 | TTA |
| 28 | E51SaCas9KKH | TTTTTTCTCATACCTTCTG | 62 | E51SaCas9KKH | CCGACTGGCTTTCTCTGCT |
| | 2 | CTT | | 36 | TGA |
| 28 | E51SaCas9KKH | TTTTTTCTCATACCTTCTG | 63 | E51SaCas9KKH | ACTTACCGACTGGCTTTCT |
| | 2 | CTT | | 37 | CTG |
| 28 | E51SaCas9KKH | TTTTTTCTCATACCTTCTG | 64 | E51SaCas9KKH | GATGTTGGAGGTACCTGCT |
| | 2 | CTT | | 38 | CTG |
| 28 | E51SaCas9KKH | TTTTTTCTCATACCTTCTG | 65 | E51SaCas9KKH | AAATGCCATCTTCCTTGAT |
| | 2 | CTT | | 39 | GTT |
| 28 | E51SaCas9KKH | TTTTTTCTCATACCTTCTG | 66 | E51SaCas9KKH | CCAAACTAGAAATGCCATC |
| | 2 | CTT | | 40 | TTC |
| 28 | E51SaCas9KKH | TTTTTTCTCATACCTTCTG | 67 | E51SaCas9KKH | AGGAAACTGCCATCTCCAA |
| | 2 | CTT | | 41 | ACT |
| 28 | E51SaCas9KKH | TTTTTTCTCATACCTTCTG | 68 | E51SaCas9KKH | CTGTTACTCTGGTGACACA |
| | 2 | CTT | | 42 | ACC |
| 28 | E51SaCas9KKH | TTTTTTCTCATACCTTCTG | 69 | E51SaCas9KKH | TAGCTCCTACTCAGACTGT |
| | 2 | CTT | | 43 | TAC |
| 29 | E51SaCas9KKH | CCTTCTGCTTGATGATCAT | 30 | E51SaCas9KKH | ATGATCATCTCGTTGATAT |
| | 3 | CTC | | 4 | CCT |
| 29 | E51SaCas9KKH | CCTTCTGCTTGATGATCAT | 31 | E51SaCas9KKH | AAGGTCACCCACCATCACC |
| | 3 | CTC | | 5 | CTC |
| 29 | E51SaCas9KKH | CCTTCTGCTTGATGATCAT | 32 | E51SaCas9KKH | ATCACCCTCTGTGATTTTA |
| | 3 | CTC | | 6 | TAA |
| 29 | E51SaCas9KKH | CCTTCTGCTTGATGATCAT | 33 | E51SaCas9KKH | ATAACTTGATCAAGCAGAG |
| | 3 | CTC | | 7 | AAA |
| 29 | E51SaCas9KKH | CCTTCTGCTTGATGATCAT | 34 | E51SaCas9KKH | CTTGATCAAGCAGAGAAAG |
| | 3 | CTC | | 8 | CCA |
| 29 | E51SaCas9KKH | CCTTCTGCTTGATGATCAT | 35 | E51SaCas9KKH | ATCAAGCAGAGAAAGCCAG |
| | 3 | CTC | | 9 | TCG |
| 29 | E51SaCas9KKH | CCTTCTGCTTGATGATCAT | 36 | E51SaCas9KKH | CAGTCGGTAAGTTCTGTCC |
| | 3 | CTC | | 10 | AAG |
| 29 | E51SaCas9KKH | CCTTCTGCTTGATGATCAT | 37 | E51SaCas9KKH | GTAAGTTCTGTCCAAGCCC |
| | 3 | CTC | | 11 | GGT |
| 29 | E51SaCas9KKH | CCTTCTGCTTGATGATCAT | 38 | E51SaCas9KKH | AGCCCGGTTGAAATCTGCC |
| | 3 | CTC | | 12 | AGA |
| 29 | E51SaCas9KKH | CCTTCTGCTTGATGATCAT | 39 | E51SaCas9KKH | AGCAGGTACCTCCAACATC |
| | 3 | CTC | | 13 | AAG |
| 29 | E51SaCas9KKH | CCTTCTGCTTGATGATCAT | 40 | E51SaCas9KKH | CAACATCAAGGAAGATGGC |
| | 3 | CTC | | 14 | ATT |
| 29 | E51SaCas9KKH | CCTTCTGCTTGATGATCAT | 41 | E51SaCas9KKH | AGGAAGATGGCATTTCTAG |
| | 3 | CTC | | 15 | TTT |
| 29 | E51SaCas9KKH | CCTTCTGCTTGATGATCAT | 42 | E51SaCas9KKH | ATGGCATTTCTAGTTTGGA |
| | 3 | CTC | | 16 | GAT |
| 29 | E51SaCas9KKH | CCTTCTGCTTGATGATCAT | 43 | E51SaCas9KKH | CTAGTTTGGAGATGGCAGT |
| | 3 | CTC | | 17 | TTC |
| 29 | E51SaCas9KKH | CCTTCTGCTTGATGATCAT | 44 | E51SaCas9KKH | GATGGCAGTTTCCTTAGTA |
| | 3 | CTC | | 18 | ACC |
| 29 | E51SaCas9KKH | CCTTCTGCTTGATGATCAT | 45 | E51SaCas9KKH | TAGTAACCACAGGTTGTGT |
| | 3 | CTC | | 19 | CAC |
| 29 | E51SaCas9KKH | CCTTCTGCTTGATGATCAT | 46 | E51SaCas9KKH | CCACAGGTTGTGTCACCAG |
| | 3 | CTC | | 20 | AGT |
| 29 | E51SaCas9KKH | CCTTCTGCTTGATGATCAT | 47 | E51SaCas9KKH | GTTGTGTCACCAGAGTAAC |
| | 3 | CTC | | 21 | AGT |
| 29 | E51SaCas9KKH | CCTTCTGCTTGATGATCAT | 48 | E51SaCas9KKH | GAGTAACAGTCTGAGTAGG |
| | 3 | CTC | | 22 | AGC |
| 29 | E51SaCas9KKH | CCTTCTGCTTGATGATCAT | 53 | E51SaCas9KKH | TCAACGAGATGATCATCAA |
| | 3 | CTC | | 27 | GCA |
| 29 | E51SaCas9KKH | CCTTCTGCTTGATGATCAT | 54 | E51SaCas9KKH | GTGACCTTGAGGATATCAA |
| | 3 | CTC | | 28 | CGA |
| 29 | E51SaCas9KKH | CCTTCTGCTTGATGATCAT | 55 | E51SaCas9KKH | TGGGTGACCTTGAGGATAT |
| | 3 | CTC | | 29 | CAA |
| 29 | E51SaCas9KKH | CCTTCTGCTTGATGATCAT | 56 | E51SaCas9KKH | GAGGGTGATGGTGGGTGAC |
| | 3 | CTC | | 30 | CTT |
| 29 | E51SaCas9KKH | CCTTCTGCTTGATGATCAT | 57 | E51SaCas9KKH | TATAAAATCACAGAGGGTG |
| | 3 | CTC | | 31 | ATG |
| 29 | E51SaCas9KKH | CCTTCTGCTTGATGATCAT | 58 | E51SaCas9KKH | AAGTTATAAAATCACAGAG |
| | 3 | CTC | | 32 | GGT |
| 29 | E51SaCas9KKH | CCTTCTGCTTGATGATCAT | 59 | E51SaCas9KKH | ATCAAGTTATAAAATCACA |
| | 3 | CTC | | 33 | GAG |
| 29 | E51SaCas9KKH | CCTTCTGCTTGATGATCAT | 60 | E51SaCas9KKH | TTGATCAAGTTATAAAATC |
| | 3 | CTC | | 34 | ACA |
| 29 | E51SaCas9KKH | CCTTCTGCTTGATGATCAT | 61 | E51SaCas9KKH | CTTTCTCTGCTTGATCAAG |
| | 3 | CTC | | 35 | TTA |
| 29 | E51SaCas9KKH | CCTTCTGCTTGATGATCAT | 62 | E51SaCas9KKH | CCGACTGGCTTTCTCTGCT |
| | 3 | CTC | | 36 | TGA |
| 29 | E51SaCas9KKH | CCTTCTGCTTGATGATCAT | 63 | E51SaCas9KKH | ACTTACCGACTGGCTTTCT |
| | 3 | CTC | | 37 | CTG |
| 29 | E51SaCas9KKH | CCTTCTGCTTGATGATCAT | 64 | E51SaCas9KKH | GATGTTGGAGGTACCTGCT |
| | 3 | CTC | | 38 | CTG |
| 29 | E51SaCas9KKH | CCTTCTGCTTGATGATCAT | 65 | E51SaCas9KKH | AAATGCCATCTTCCTTGAT |
| | 3 | CTC | | 39 | GTT |
| 29 | E51SaCas9KKH | CCTTCTGCTTGATGATCAT | 66 | E51SaCas9KKH | CCAAACTAGAAATGCCATC |
| | 3 | CTC | | 40 | TTC |
| 29 | E51SaCas9KKH | CCTTCTGCTTGATGATCAT | 67 | E51SaCas9KKH | AGGAAACTGCCATCTCCAA |
| | 3 | CTC | | 41 | ACT |
| 29 | E51SaCas9KKH | CCTTCTGCTTGATGATCAT | 68 | E51SaCas9KKH | CTGTTACTCTGGTGACACA |
| | 3 | CTC | | 42 | ACC |
| 29 | E51SaCas9KKH | CCTTCTGCTTGATGATCAT | 69 | E51SaCas9KKH | TAGCTCCTACTCAGACTGT |
| | 3 | CTC | | 43 | TAC |
| 30 | E51SaCas9KKH | ATGATCATCTCGTTGATAT | 31 | E51SaCas9KKH | AAGGTCACCCACCATCACC |
| | 4 | CCT | | 5 | CTC |
| 30 | E51SaCas9KKH | ATGATCATCTCGTTGATAT | 32 | E51SaCas9KKH | ATCACCCTCTGTGATTTTA |
| | 4 | CCT | | 6 | TAA |
| 30 | E51SaCas9KKH | ATGATCATCTCGTTGATAT | 33 | E51SaCas9KKH | ATAACTTGATCAAGCAGAG |
| | 4 | CCT | | 7 | AAA |
| 30 | E51SaCas9KKH | ATGATCATCTCGTTGATAT | 34 | E51SaCas9KKH | CTTGATCAAGCAGAGAAAG |
| | 4 | CCT | | 8 | CCA |
| 30 | E51SaCas9KKH | ATGATCATCTCGTTGATAT | 35 | E51SaCas9KKH | ATCAAGCAGAGAAAGCCAG |
| | 4 | CCT | | 9 | TCG |
| 30 | E51SaCas9KKH | ATGATCATCTCGTTGATAT | 36 | E51SaCas9KKH | CAGTCGGTAAGTTCTGTCC |
| | 4 | CCT | | 10 | AAG |
| 30 | E51SaCas9KKH | ATGATCATCTCGTTGATAT | 37 | E51SaCas9KKH | GTAAGTTCTGTCCAAGCCC |
| | 4 | CCT | | 11 | GGT |
| 30 | E51SaCas9KKH | ATGATCATCTCGTTGATAT | 38 | E51SaCas9KKH | AGCCCGGTTGAAATCTGCC |
| | 4 | CCT | | 12 | AGA |
| 30 | E51SaCas9KKH | ATGATCATCTCGTTGATAT | 39 | E51SaCas9KKH | AGCAGGTACCTCCAACATC |
| | 4 | CCT | | 13 | AAG |
| 30 | E51SaCas9KKH | ATGATCATCTCGTTGATAT | 40 | E51SaCas9KKH | CAACATCAAGGAAGATGGC |
| | 4 | CCT | | 14 | ATT |
| 30 | E51SaCas9KKH | ATGATCATCTCGTTGATAT | 41 | E51SaCas9KKH | AGGAAGATGGCATTTCTAG |
| | 4 | CCT | | 15 | TTT |
| 30 | E51SaCas9KKH | ATGATCATCTCGTTGATAT | 42 | E51SaCas9KKH | ATGGCATTTCTAGTTTGGA |
| | 4 | CCT | | 16 | GAT |
| 30 | E51SaCas9KKH | ATGATCATCTCGTTGATAT | 43 | E51SaCas9KKH | CTAGTTTGGAGATGGCAGT |
| | 4 | CCT | | 17 | TTC |
| 30 | E51SaCas9KKH | ATGATCATCTCGTTGATAT | 44 | E51SaCas9KKH | GATGGCAGTTTCCTTAGTA |
| | 4 | CCT | | 18 | ACC |
| 30 | E51SaCas9KKH | ATGATCATCTCGTTGATAT | 45 | E51SaCas9KKH | TAGTAACCACAGGTTGTGT |
| | 4 | CCT | | 19 | CAC |
| 30 | E51SaCas9KKH | ATGATCATCTCGTTGATAT | 46 | E51SaCas9KKH | CCACAGGTTGTGTCACCAG |
| | 4 | CCT | | 20 | AGT |
| 30 | E51SaCas9KKH | ATGATCATCTCGTTGATAT | 47 | E51SaCas9KKH | GTTGTGTCACCAGAGTAAC |
| | 4 | CCT | | 21 | AGT |
| 30 | E51SaCas9KKH | ATGATCATCTCGTTGATAT | 48 | E51SaCas9KKH | GAGTAACAGTCTGAGTAGG |
| | 4 | CCT | | 22 | AGC |
| 30 | E51SaCas9KKH | ATGATCATCTCGTTGATAT | 53 | E51SaCas9KKH | TCAACGAGATGATCATCAA |
| | 4 | CCT | | 27 | GCA |
| 30 | E51SaCas9KKH | ATGATCATCTCGTTGATAT | 54 | E51SaCas9KKH | GTGACCTTGAGGATATCAA |
| | 4 | CCT | | 28 | CGA |
| 30 | E51SaCas9KKH | ATGATCATCTCGTTGATAT | 55 | E51SaCas9KKH | TGGGTGACCTTGAGGATAT |
| | 4 | CCT | | 29 | CAA |
| 30 | E51SaCas9KKH | ATGATCATCTCGTTGATAT | 56 | E51SaCas9KKH | GAGGGTGATGGTGGGTGAC |
| | 4 | CCT | | 30 | CTT |
| 30 | E51SaCas9KKH | ATGATCATCTCGTTGATAT | 57 | E51SaCas9KKH | TATAAAATCACAGAGGGTG |
| | 4 | CCT | | 31 | ATG |
| 30 | E51SaCas9KKH | ATGATCATCTCGTTGATAT | 58 | E51SaCas9KKH | AAGTTATAAAATCACAGAG |
| | 4 | CCT | | 32 | GGT |
| 30 | E51SaCas9KKH | ATGATCATCTCGTTGATAT | 59 | E51SaCas9KKH | ATCAAGTTATAAAATCACA |
| | 4 | CCT | | 33 | GAG |
| 30 | E51SaCas9KKH | ATGATCATCTCGTTGATAT | 60 | E51SaCas9KKH | TTGATCAAGTTATAAAATC |
| | 4 | CCT | | 34 | ACA |
| 30 | E51SaCas9KKH | ATGATCATCTCGTTGATAT | 61 | E51SaCas9KKH | CTTTCTCTGCTTGATCAAG |
| | 4 | CCT | | 35 | TTA |
| 30 | E51SaCas9KKH | ATGATCATCTCGTTGATAT | 62 | E51SaCas9KKH | CCGACTGGCTTTCTCTGCT |
| | 4 | CCT | | 36 | TGA |
| 30 | E51SaCas9KKH | ATGATCATCTCGTTGATAT | 63 | E51SaCas9KKH | ACTTACCGACTGGCTTTCT |
| | 4 | CCT | | 37 | CTG |
| 30 | E51SaCas9KKH | ATGATCATCTCGTTGATAT | 64 | E51SaCas9KKH | GATGTTGGAGGTACCTGCT |
| | 4 | CCT | | 38 | CTG |
| 30 | E51SaCas9KKH | ATGATCATCTCGTTGATAT | 65 | E51SaCas9KKH | AAATGCCATCTTCCTTGAT |
| | 4 | CCT | | 39 | GTT |
| 30 | E51SaCas9KKH | ATGATCATCTCGTTGATAT | 66 | E51SaCas9KKH | CCAAACTAGAAATGCCATC |
| | 4 | CCT | | 40 | TTC |
| 30 | E51SaCas9KKH | ATGATCATCTCGTTGATAT | 67 | E51SaCas9KKH | AGGAAACTGCCATCTCCAA |
| | 4 | CCT | | 41 | ACT |
| 30 | E51SaCas9KKH | ATGATCATCTCGTTGATAT | 68 | E51SaCas9KKH | CTGTTACTCTGGTGACACA |
| | 4 | CCT | | 42 | ACC |
| 30 | E51SaCas9KKH | ATGATCATCTCGTTGATAT | 69 | E51SaCas9KKH | TAGCTCCTACTCAGACTGT |
| | 4 | CCT | | 43 | TAC |
| 31 | E51SaCas9KKH | AAGGTCACCCACCATCACC | 32 | E51SaCas9KKH | ATCACCCTCTGTGATTTTA |
| | 5 | CTC | | 6 | TAA |
| 31 | E51SaCas9KKH | AAGGTCACCCACCATCACC | 33 | E51SaCas9KKH | ATAACTTGATCAAGCAGAG |
| | 5 | CTC | | 7 | AAA |
| 31 | E51SaCas9KKH | AAGGTCACCCACCATCACC | 34 | E51SaCas9KKH | CTTGATCAAGCAGAGAAAG |
| | 5 | CTC | | 8 | CCA |
| 31 | E51SaCas9KKH | AAGGTCACCCACCATCACC | 35 | E51SaCas9KKH | ATCAAGCAGAGAAAGCCAG |
| | 5 | CTC | | 9 | TCG |
| 31 | E51SaCas9KKH | AAGGTCACCCACCATCACC | 36 | E51SaCas9KKH | CAGTCGGTAAGTTCTGTCC |
| | 5 | CTC | | 10 | AAG |
| 31 | E51SaCas9KKH | AAGGTCACCCACCATCACC | 37 | E51SaCas9KKH | GTAAGTTCTGTCCAAGCCC |
| | 5 | CTC | | 11 | GGT |
| 31 | E51SaCas9KKH | AAGGTCACCCACCATCACC | 38 | E51SaCas9KKH | AGCCCGGTTGAAATCTGCC |
| | 5 | CTC | | 12 | AGA |
| 31 | E51SaCas9KKH | AAGGTCACCCACCATCACC | 39 | E51SaCas9KKH | AGCAGGTACCTCCAACATC |
| | 5 | CTC | | 13 | AAG |
| 31 | E51SaCas9KKH | AAGGTCACCCACCATCACC | 40 | E51SaCas9KKH | CAACATCAAGGAAGATGGC |
| | 5 | CTC | | 14 | ATT |
| 31 | E51SaCas9KKH | AAGGTCACCCACCATCACC | 41 | E51SaCas9KKH | AGGAAGATGGCATTTCTAG |
| | 5 | CTC | | 15 | TTT |
| 31 | E51SaCas9KKH | AAGGTCACCCACCATCACC | 42 | E51SaCas9KKH | ATGGCATTTCTAGTTTGGA |
| | 5 | CTC | | 16 | GAT |
| 31 | E51SaCas9KKH | AAGGTCACCCACCATCACC | 43 | E51SaCas9KKH | CTAGTTTGGAGATGGCAGT |
| | 5 | CTC | | 17 | TTC |
| 31 | E51SaCas9KKH | AAGGTCACCCACCATCACC | 44 | E51SaCas9KKH | GATGGCAGTTTCCTTAGTA |
| | 5 | CTC | | 18 | ACC |
| 31 | E51SaCas9KKH | AAGGTCACCCACCATCACC | 45 | E51SaCas9KKH | TAGTAACCACAGGTTGTGT |
| | 5 | CTC | | 19 | CAC |
| 31 | E51SaCas9KKH | AAGGTCACCCACCATCACC | 46 | E51SaCas9KKH | CCACAGGTTGTGTCACCAG |
| | 5 | CTC | | 20 | AGT |
| 31 | E51SaCas9KKH | AAGGTCACCCACCATCACC | 47 | E51SaCas9KKH | GTTGTGTCACCAGAGTAAC |
| | 5 | CTC | | 21 | AGT |
| 31 | E51SaCas9KKH | AAGGTCACCCACCATCACC | 48 | E51SaCas9KKH | GAGTAACAGTCTGAGTAGG |
| | 5 | CTC | | 22 | AGC |
| 31 | E51SaCas9KKH | AAGGTCACCCACCATCACC | 53 | E51SaCas9KKH | TCAACGAGATGATCATCAA |
| | 5 | CTC | | 27 | GCA |
| 31 | E51SaCas9KKH | AAGGTCACCCACCATCACC | 54 | E51SaCas9KKH | GTGACCTTGAGGATATCAA |
| | 5 | CTC | | 28 | CGA |
| 31 | E51SaCas9KKH | AAGGTCACCCACCATCACC | 55 | E51SaCas9KKH | TGGGTGACCTTGAGGATAT |
| | 5 | CTC | | 29 | CAA |
| 31 | E51SaCas9KKH | AAGGTCACCCACCATCACC | 56 | E51SaCas9KKH | GAGGGTGATGGTGGGTGAC |
| | 5 | CTC | | 30 | CTT |
| 31 | E51SaCas9KKH | AAGGTCACCCACCATCACC | 57 | E51SaCas9KKH | TATAAAATCACAGAGGGTG |
| | 5 | CTC | | 31 | ATG |
| 31 | E51SaCas9KKH | AAGGTCACCCACCATCACC | 58 | E51SaCas9KKH | AAGTTATAAAATCACAGAG |
| | 5 | CTC | | 32 | GGT |
| 31 | E51SaCas9KKH | AAGGTCACCCACCATCACC | 59 | E51SaCas9KKH | ATCAAGTTATAAAATCACA |
| | 5 | CTC | | 33 | GAG |
| 31 | E51SaCas9KKH | AAGGTCACCCACCATCACC | 60 | E51SaCas9KKH | TTGATCAAGTTATAAAATC |
| | 5 | CTC | | 34 | ACA |
| 31 | E51SaCas9KKH | AAGGTCACCCACCATCACC | 61 | E51SaCas9KKH | CTTTCTCTGCTTGATCAAG |
| | 5 | CTC | | 35 | TTA |
| 31 | E51SaCas9KKH | AAGGTCACCCACCATCACC | 62 | E51SaCas9KKH | CCGACTGGCTTTCTCTGCT |
| | 5 | CTC | | 36 | TGA |
| 31 | E51SaCas9KKH | AAGGTCACCCACCATCACC | 63 | E51SaCas9KKH | ACTTACCGACTGGCTTTCT |
| | 5 | CTC | | 37 | CTG |
| 31 | E51SaCas9KKH | AAGGTCACCCACCATCACC | 64 | E51SaCas9KKH | GATGTTGGAGGTACCTGCT |
| | 5 | CTC | | 38 | CTG |
| 31 | E51SaCas9KKH | AAGGTCACCCACCATCACC | 65 | E51SaCas9KKH | AAATGCCATCTTCCTTGAT |
| | 5 | CTC | | 39 | GTT |
| 31 | E51SaCas9KKH | AAGGTCACCCACCATCACC | 66 | E51SaCas9KKH | CCAAACTAGAAATGCCATC |
| | 5 | CTC | | 40 | TTC |
| 31 | E51SaCas9KKH | AAGGTCACCCACCATCACC | 67 | E51SaCas9KKH | AGGAAACTGCCATCTCCAA |
| | 5 | CTC | | 41 | ACT |
| 31 | E51SaCas9KKH | AAGGTCACCCACCATCACC | 68 | E51SaCas9KKH | CTGTTACTCTGGTGACACA |
| | 5 | CTC | | 42 | ACC |
| 31 | E51SaCas9KKH | AAGGTCACCCACCATCACC | 69 | E51SaCas9KKH | TAGCTCCTACTCAGACTGT |
| | 5 | CTC | | 43 | TAC |
| 32 | E51SaCas9KKH | ATCACCCTCTGTGATTTTA | 33 | E51SaCas9KKH | ATAACTTGATCAAGCAGAG |
| | 6 | TAA | | 7 | AAA |
| 32 | E51SaCas9KKH | ATCACCCTCTGTGATTTTA | 34 | E51SaCas9KKH | CTTGATCAAGCAGAGAAAG |
| | 6 | TAA | | 8 | CCA |
| 32 | E51SaCas9KKH | ATCACCCTCTGTGATTTTA | 35 | E51SaCas9KKH | ATCAAGCAGAGAAAGCCAG |
| | 6 | TAA | | 9 | TCG |
| 32 | E51SaCas9KKH | ATCACCCTCTGTGATTTTA | 36 | E51SaCas9KKH | CAGTCGGTAAGTTCTGTCC |
| | 6 | TAA | | 10 | AAG |
| 32 | E51SaCas9KKH | ATCACCCTCTGTGATTTTA | 37 | E51SaCas9KKH | GTAAGTTCTGTCCAAGCCC |
| | 6 | TAA | | 11 | GGT |
| 32 | E51SaCas9KKH | ATCACCCTCTGTGATTTTA | 38 | E51SaCas9KKH | AGCCCGGTTGAAATCTGCC |
| | 6 | TAA | | 12 | AGA |
| 32 | E51SaCas9KKH | ATCACCCTCTGTGATTTTA | 39 | E51SaCas9KKH | AGCAGGTACCTCCAACATC |
| | 6 | TAA | | 13 | AAG |
| 32 | E51SaCas9KKH | ATCACCCTCTGTGATTTTA | 40 | E51SaCas9KKH | CAACATCAAGGAAGATGGC |
| | 6 | TAA | | 14 | ATT |
| 32 | E51SaCas9KKH | ATCACCCTCTGTGATTTTA | 41 | E51SaCas9KKH | AGGAAGATGGCATTTCTAG |
| | 6 | TAA | | 15 | TTT |
| 32 | E51SaCas9KKH | ATCACCCTCTGTGATTTTA | 42 | E51SaCas9KKH | ATGGCATTTCTAGTTTGGA |
| | 6 | TAA | | 16 | GAT |
| 32 | E51SaCas9KKH | ATCACCCTCTGTGATTTTA | 43 | E51SaCas9KKH | CTAGTTTGGAGATGGCAGT |
| | 6 | TAA | | 17 | TTC |
| 32 | E51SaCas9KKH | ATCACCCTCTGTGATTTTA | 44 | E51SaCas9KKH | GATGGCAGTTTCCTTAGTA |
| | 6 | TAA | | 18 | ACC |
| 32 | E51SaCas9KKH | ATCACCCTCTGTGATTTTA | 45 | E51SaCas9KKH | TAGTAACCACAGGTTGTGT |
| | 6 | TAA | | 19 | CAC |
| 32 | E51SaCas9KKH | ATCACCCTCTGTGATTTTA | 46 | E51SaCas9KKH | CCACAGGTTGTGTCACCAG |
| | 6 | TAA | | 20 | AGT |
| 32 | E51SaCas9KKH | ATCACCCTCTGTGATTTTA | 47 | E51SaCas9KKH | GTTGTGTCACCAGAGTAAC |
| | 6 | TAA | | 21 | AGT |
| 32 | E51SaCas9KKH | ATCACCCTCTGTGATTTTA | 48 | E51SaCas9KKH | GAGTAACAGTCTGAGTAGG |
| | 6 | TAA | | 22 | AGC |
| 32 | E51SaCas9KKH | ATCACCCTCTGTGATTTTA | 53 | E51SaCas9KKH | TCAACGAGATGATCATCAA |
| | 6 | TAA | | 27 | GCA |
| 32 | E51SaCas9KKH | ATCACCCTCTGTGATTTTA | 54 | E51SaCas9KKH | GTGACCTTGAGGATATCAA |
| | 6 | TAA | | 28 | CGA |
| 32 | E51SaCas9KKH | ATCACCCTCTGTGATTTTA | 55 | E51SaCas9KKH | TGGGTGACCTTGAGGATAT |
| | 6 | TAA | | 29 | CAA |
| 32 | E51SaCas9KKH | ATCACCCTCTGTGATTTTA | 56 | E51SaCas9KKH | GAGGGTGATGGTGGGTGAC |
| | 6 | TAA | | 30 | CTT |
| 32 | E51SaCas9KKH | ATCACCCTCTGTGATTTTA | 57 | E51SaCas9KKH | TATAAAATCACAGAGGGTG |
| | 6 | TAA | | 31 | ATG |
| 32 | E51SaCas9KKH | ATCACCCTCTGTGATTTTA | 58 | E51SaCas9KKH | AAGTTATAAAATCACAGAG |
| | 6 | TAA | | 32 | GGT |
| 32 | E51SaCas9KKH | ATCACCCTCTGTGATTTTA | 59 | E51SaCas9KKH | ATCAAGTTATAAAATCACA |
| | 6 | TAA | | 33 | GAG |
| 32 | E51SaCas9KKH | ATCACCCTCTGTGATTTTA | 60 | E51SaCas9KKH | TTGATCAAGTTATAAAATC |
| | 6 | TAA | | 34 | ACA |
| 32 | E51SaCas9KKH | ATCACCCTCTGTGATTTTA | 61 | E51SaCas9KKH | CTTTCTCTGCTTGATCAAG |
| | 6 | TAA | | 35 | TTA |
| 32 | E51SaCas9KKH | ATCACCCTCTGTGATTTTA | 62 | E51SaCas9KKH | CCGACTGGCTTTCTCTGCT |
| | 6 | TAA | | 36 | TGA |
| 32 | E51SaCas9KKH | ATCACCCTCTGTGATTTTA | 63 | E51SaCas9KKH | ACTTACCGACTGGCTTTCT |
| | 6 | TAA | | 37 | CTG |
| 32 | E51SaCas9KKH | ATCACCCTCTGTGATTTTA | 64 | E51SaCas9KKH | GATGTTGGAGGTACCTGCT |
| | 6 | TAA | | 38 | CTG |
| 32 | E51SaCas9KKH | ATCACCCTCTGTGATTTTA | 65 | E51SaCas9KKH | AAATGCCATCTTCCTTGAT |
| | 6 | TAA | | 39 | GTT |
| 32 | E51SaCas9KKH | ATCACCCTCTGTGATTTTA | 66 | E51SaCas9KKH | CCAAACTAGAAATGCCATC |
| | 6 | TAA | | 40 | TTC |
| 32 | E51SaCas9KKH | ATCACCCTCTGTGATTTTA | 67 | E51SaCas9KKH | AGGAAACTGCCATCTCCAA |
| | 6 | TAA | | 41 | ACT |
| 32 | E51SaCas9KKH | ATCACCCTCTGTGATTTTA | 68 | E51SaCas9KKH | CTGTTACTCTGGTGACACA |
| | 6 | TAA | | 42 | ACC |
| 32 | E51SaCas9KKH | ATCACCCTCTGTGATTTTA | 69 | E51SaCas9KKH | TAGCTCCTACTCAGACTGT |
| | 6 | TAA | | 43 | TAC |
| 33 | E51SaCas9KKH | ATAACTTGATCAAGCAGAG | 34 | E51SaCas9KKH | CTTGATCAAGCAGAGAAAG |
| | 7 | AAA | | 8 | CCA |
| 33 | E51SaCas9KKH | ATAACTTGATCAAGCAGAG | 35 | E51SaCas9KKH | ATCAAGCAGAGAAAGCCAG |
| | 7 | AAA | | 9 | TCG |
| 33 | E51SaCas9KKH | ATAACTTGATCAAGCAGAG | 36 | E51SaCas9KKH | CAGTCGGTAAGTTCTGTCC |
| | 7 | AAA | | 10 | AAG |
| 33 | E51SaCas9KKH | ATAACTTGATCAAGCAGAG | 37 | E51SaCas9KKH | GTAAGTTCTGTCCAAGCCC |
| | 7 | AAA | | 11 | GGT |
| 33 | E51SaCas9KKH | ATAACTTGATCAAGCAGAG | 38 | E51SaCas9KKH | AGCCCGGTTGAAATCTGCC |
| | 7 | AAA | | 12 | AGA |
| 33 | E51SaCas9KKH | ATAACTTGATCAAGCAGAG | 39 | E51SaCas9KKH | AGCAGGTACCTCCAACATC |
| | 7 | AAA | | 13 | AAG |
| 33 | E51SaCas9KKH | ATAACTTGATCAAGCAGAG | 40 | E51SaCas9KKH | CAACATCAAGGAAGATGGC |
| | 7 | AAA | | 14 | ATT |
| 33 | E51SaCas9KKH | ATAACTTGATCAAGCAGAG | 41 | E51SaCas9KKH | AGGAAGATGGCATTTCTAG |
| | 7 | AAA | | 15 | TTT |
| 33 | E51SaCas9KKH | ATAACTTGATCAAGCAGAG | 42 | E51SaCas9KKH | ATGGCATTTCTAGTTTGGA |
| | 7 | AAA | | 16 | GAT |
| 33 | E51SaCas9KKH | ATAACTTGATCAAGCAGAG | 43 | E51SaCas9KKH | CTAGTTTGGAGATGGCAGT |
| | 7 | AAA | | 17 | TTC |
| 33 | E51SaCas9KKH | ATAACTTGATCAAGCAGAG | 44 | E51SaCas9KKH | GATGGCAGTTTCCTTAGTA |
| | 7 | AAA | | 18 | ACC |
| 33 | E51SaCas9KKH | ATAACTTGATCAAGCAGAG | 45 | E51SaCas9KKH | TAGTAACCACAGGTTGTGT |
| | 7 | AAA | | 19 | CAC |
| 33 | E51SaCas9KKH | ATAACTTGATCAAGCAGAG | 46 | E51SaCas9KKH | CCACAGGTTGTGTCACCAG |
| | 7 | AAA | | 20 | AGT |
| 33 | E51SaCas9KKH | ATAACTTGATCAAGCAGAG | 47 | E51SaCas9KKH | GTTGTGTCACCAGAGTAAC |
| | 7 | AAA | | 21 | AGT |
| 33 | E51SaCas9KKH | ATAACTTGATCAAGCAGAG | 48 | E51SaCas9KKH | GAGTAACAGTCTGAGTAGG |
| | 7 | AAA | | 22 | AGC |
| 33 | E51SaCas9KKH | ATAACTTGATCAAGCAGAG | 53 | E51SaCas9KKH | TCAACGAGATGATCATCAA |
| | 7 | AAA | | 27 | GCA |
| 33 | E51SaCas9KKH | ATAACTTGATCAAGCAGAG | 54 | E51SaCas9KKH | GTGACCTTGAGGATATCAA |
| | 7 | AAA | | 28 | CGA |
| 33 | E51SaCas9KKH | ATAACTTGATCAAGCAGAG | 55 | E51SaCas9KKH | TGGGTGACCTTGAGGATAT |
| | 7 | AAA | | 29 | CAA |
| 33 | E51SaCas9KKH | ATAACTTGATCAAGCAGAG | 56 | E51SaCas9KKH | GAGGGTGATGGTGGGTGAC |
| | 7 | AAA | | 30 | CTT |
| 33 | E51SaCas9KKH | ATAACTTGATCAAGCAGAG | 57 | E51SaCas9KKH | TATAAAATCACAGAGGGTG |
| | 7 | AAA | | 31 | ATG |
| 33 | E51SaCas9KKH | ATAACTTGATCAAGCAGAG | 58 | E51SaCas9KKH | AAGTTATAAAATCACAGAG |
| | 7 | AAA | | 32 | GGT |
| 33 | E51SaCas9KKH | ATAACTTGATCAAGCAGAG | 59 | E51SaCas9KKH | ATCAAGTTATAAAATCACA |
| | 7 | AAA | | 33 | GAG |
| 33 | E51SaCas9KKH | ATAACTTGATCAAGCAGAG | 60 | E51SaCas9KKH | TTGATCAAGTTATAAAATC |
| | 7 | AAA | | 34 | ACA |
| 33 | E51SaCas9KKH | ATAACTTGATCAAGCAGAG | 61 | E51SaCas9KKH | CTTTCTCTGCTTGATCAAG |
| | 7 | AAA | | 35 | TTA |
| 33 | E51SaCas9KKH | ATAACTTGATCAAGCAGAG | 62 | E51SaCas9KKH | CCGACTGGCTTTCTCTGCT |
| | 7 | AAA | | 36 | TGA |
| 33 | E51SaCas9KKH | ATAACTTGATCAAGCAGAG | 63 | E51SaCas9KKH | ACTTACCGACTGGCTTTCT |
| | 7 | AAA | | 37 | CTG |
| 33 | E51SaCas9KKH | ATAACTTGATCAAGCAGAG | 64 | E51SaCas9KKH | GATGTTGGAGGTACCTGCT |
| | 7 | AAA | | 38 | CTG |
| 33 | E51SaCas9KKH | ATAACTTGATCAAGCAGAG | 65 | E51SaCas9KKH | AAATGCCATCTTCCTTGAT |
| | 7 | AAA | | 39 | GTT |
| 33 | E51SaCas9KKH | ATAACTTGATCAAGCAGAG | 66 | E51SaCas9KKH | CCAAACTAGAAATGCCATC |
| | 7 | AAA | | 40 | TTC |
| 33 | E51SaCas9KKH | ATAACTTGATCAAGCAGAG | 67 | E51SaCas9KKH | AGGAAACTGCCATCTCCAA |
| | 7 | AAA | | 41 | ACT |
| 33 | E51SaCas9KKH | ATAACTTGATCAAGCAGAG | 68 | E51SaCas9KKH | CTGTTACTCTGGTGACACA |
| | 7 | AAA | | 42 | ACC |
| 33 | E51SaCas9KKH | ATAACTTGATCAAGCAGAG | 69 | E51SaCas9KKH | TAGCTCCTACTCAGACTGT |
| | 7 | AAA | | 43 | TAC |
| 34 | E51SaCas9KKH | CTTGATCAAGCAGAGAAAG | 35 | E51SaCas9KKH | ATCAAGCAGAGAAAGCCAG |
| | 8 | CCA | | 9 | TCG |
| 34 | E51SaCas9KKH | CTTGATCAAGCAGAGAAAG | 36 | E51SaCas9KKH | CAGTCGGTAAGTTCTGTCC |
| | 8 | CCA | | 10 | AAG |
| 34 | E51SaCas9KKH | CTTGATCAAGCAGAGAAAG | 37 | E51SaCas9KKH | GTAAGTTCTGTCCAAGCCC |
| | 8 | CCA | | 11 | GGT |
| 34 | E51SaCas9KKH | CTTGATCAAGCAGAGAAAG | 38 | E51SaCas9KKH | AGCCCGGTTGAAATCTGCC |
| | 8 | CCA | | 12 | AGA |
| 34 | E51SaCas9KKH | CTTGATCAAGCAGAGAAAG | 39 | E51SaCas9KKH | AGCAGGTACCTCCAACATC |
| | 8 | CCA | | 13 | AAG |
| 34 | E51SaCas9KKH | CTTGATCAAGCAGAGAAAG | 40 | E51SaCas9KKH | CAACATCAAGGAAGATGGC |
| | 8 | CCA | | 14 | ATT |
| 34 | E51SaCas9KKH | CTTGATCAAGCAGAGAAAG | 41 | E51SaCas9KKH | AGGAAGATGGCATTTCTAG |
| | 8 | CCA | | 15 | TTT |
| 34 | E51SaCas9KKH | CTTGATCAAGCAGAGAAAG | 42 | E51SaCas9KKH | ATGGCATTTCTAGTTTGGA |
| | 8 | CCA | | 16 | GAT |
| 34 | E51SaCas9KKH | CTTGATCAAGCAGAGAAAG | 43 | E51SaCas9KKH | CTAGTTTGGAGATGGCAGT |
| | 8 | CCA | | 17 | TTC |
| 34 | E51SaCas9KKH | CTTGATCAAGCAGAGAAAG | 44 | E51SaCas9KKH | GATGGCAGTTTCCTTAGTA |
| | 8 | CCA | | 18 | ACC |
| 34 | E51SaCas9KKH | CTTGATCAAGCAGAGAAAG | 45 | E51SaCas9KKH | TAGTAACCACAGGTTGTGT |
| | 8 | CCA | | 19 | CAC |
| 34 | E51SaCas9KKH | CTTGATCAAGCAGAGAAAG | 46 | E51SaCas9KKH | CCACAGGTTGTGTCACCAG |
| | 8 | CCA | | 20 | AGT |
| 34 | E51SaCas9KKH | CTTGATCAAGCAGAGAAAG | 47 | E51SaCas9KKH | GTTGTGTCACCAGAGTAAC |
| | 8 | CCA | | 21 | AGT |
| 34 | E51SaCas9KKH | CTTGATCAAGCAGAGAAAG | 48 | E51SaCas9KKH | GAGTAACAGTCTGAGTAGG |
| | 8 | CCA | | 22 | AGC |
| 34 | E51SaCas9KKH | CTTGATCAAGCAGAGAAAG | 53 | E51SaCas9KKH | TCAACGAGATGATCATCAA |
| | 8 | CCA | | 27 | GCA |
| 34 | E51SaCas9KKH | CTTGATCAAGCAGAGAAAG | 54 | E51SaCas9KKH | GTGACCTTGAGGATATCAA |
| | 8 | CCA | | 28 | CGA |
| 34 | E51SaCas9KKH | CTTGATCAAGCAGAGAAAG | 55 | E51SaCas9KKH | TGGGTGACCTTGAGGATAT |
| | 8 | CCA | | 29 | CAA |
| 34 | E51SaCas9KKH | CTTGATCAAGCAGAGAAAG | 56 | E51SaCas9KKH | GAGGGTGATGGTGGGTGAC |
| | 8 | CCA | | 30 | CTT |
| 34 | E51SaCas9KKH | CTTGATCAAGCAGAGAAAG | 57 | E51SaCas9KKH | TATAAAATCACAGAGGGTG |
| | 8 | CCA | | 31 | ATG |
| 34 | E51SaCas9KKH | CTTGATCAAGCAGAGAAAG | 58 | E51SaCas9KKH | AAGTTATAAAATCACAGAG |
| | 8 | CCA | | 32 | GGT |
| 34 | E51SaCas9KKH | CTTGATCAAGCAGAGAAAG | 59 | E51SaCas9KKH | ATCAAGTTATAAAATCACA |
| | 8 | CCA | | 33 | GAG |
| 34 | E51SaCas9KKH | CTTGATCAAGCAGAGAAAG | 60 | E51SaCas9KKH | TTGATCAAGTTATAAAATC |
| | 8 | CCA | | 34 | ACA |
| 34 | E51SaCas9KKH | CTTGATCAAGCAGAGAAAG | 61 | E51SaCas9KKH | CTTTCTCTGCTTGATCAAG |
| | 8 | CCA | | 35 | TTA |
| 34 | E51SaCas9KKH | CTTGATCAAGCAGAGAAAG | 62 | E51SaCas9KKH | CCGACTGGCTTTCTCTGCT |
| | 8 | CCA | | 36 | TGA |
| 34 | E51SaCas9KKH | CTTGATCAAGCAGAGAAAG | 63 | E51SaCas9KKH | ACTTACCGACTGGCTTTCT |
| | 8 | CCA | | 37 | CTG |
| 34 | E51SaCas9KKH | CTTGATCAAGCAGAGAAAG | 64 | E51SaCas9KKH | GATGTTGGAGGTACCTGCT |
| | 8 | CCA | | 38 | CTG |
| 34 | E51SaCas9KKH | CTTGATCAAGCAGAGAAAG | 65 | E51SaCas9KKH | AAATGCCATCTTCCTTGAT |
| | 8 | CCA | | 39 | GTT |
| 34 | E51SaCas9KKH | CTTGATCAAGCAGAGAAAG | 66 | E51SaCas9KKH | CCAAACTAGAAATGCCATC |
| | 8 | CCA | | 40 | TTC |
| 34 | E51SaCas9KKH | CTTGATCAAGCAGAGAAAG | 67 | E51SaCas9KKH | AGGAAACTGCCATCTCCAA |
| | 8 | CCA | | 41 | ACT |
| 34 | E51SaCas9KKH | CTTGATCAAGCAGAGAAAG | 68 | E51SaCas9KKH | CTGTTACTCTGGTGACACA |
| | 8 | CCA | | 42 | ACC |
| 34 | E51SaCas9KKH | CTTGATCAAGCAGAGAAAG | 69 | E51SaCas9KKH | TAGCTCCTACTCAGACTGT |
| | 8 | CCA | | 43 | TAC |
| 35 | E51SaCas9KKH | ATCAAGCAGAGAAAGCCAG | 36 | E51SaCas9KKH | CAGTCGGTAAGTTCTGTCC |
| | 9 | TCG | | 10 | AAG |
| 35 | E51SaCas9KKH | ATCAAGCAGAGAAAGCCAG | 37 | E51SaCas9KKH | GTAAGTTCTGTCCAAGCCC |
| | 9 | TCG | | 11 | GGT |
| 35 | E51SaCas9KKH | ATCAAGCAGAGAAAGCCAG | 38 | E51SaCas9KKH | AGCCCGGTTGAAATCTGCC |
| | 9 | TCG | | 12 | AGA |
| 35 | E51SaCas9KKH | ATCAAGCAGAGAAAGCCAG | 39 | E51SaCas9KKH | AGCAGGTACCTCCAACATC |
| | 9 | TCG | | 13 | AAG |
| 35 | E51SaCas9KKH | ATCAAGCAGAGAAAGCCAG | 40 | E51SaCas9KKH | CAACATCAAGGAAGATGGC |
| | 9 | TCG | | 14 | ATT |
| 35 | E51SaCas9KKH | ATCAAGCAGAGAAAGCCAG | 41 | E51SaCas9KKH | AGGAAGATGGCATTTCTAG |
| | 9 | TCG | | 15 | TTT |
| 35 | E51SaCas9KKH | ATCAAGCAGAGAAAGCCAG | 42 | E51SaCas9KKH | ATGGCATTTCTAGTTTGGA |
| | 9 | TCG | | 16 | GAT |
| 35 | E51SaCas9KKH | ATCAAGCAGAGAAAGCCAG | 43 | E51SaCas9KKH | CTAGTTTGGAGATGGCAGT |
| | 9 | TCG | | 17 | TTC |
| 35 | E51SaCas9KKH | ATCAAGCAGAGAAAGCCAG | 44 | E51SaCas9KKH | GATGGCAGTTTCCTTAGTA |
| | 9 | TCG | | 18 | ACC |
| 35 | E51SaCas9KKH | ATCAAGCAGAGAAAGCCAG | 45 | E51SaCas9KKH | TAGTAACCACAGGTTGTGT |
| | 9 | TCG | | 19 | CAC |
| 35 | E51SaCas9KKH | ATCAAGCAGAGAAAGCCAG | 46 | E51SaCas9KKH | CCACAGGTTGTGTCACCAG |
| | 9 | TCG | | 20 | AGT |
| 35 | E51SaCas9KKH | ATCAAGCAGAGAAAGCCAG | 47 | E51SaCas9KKH | GTTGTGTCACCAGAGTAAC |
| | 9 | TCG | | 21 | AGT |
| 35 | E51SaCas9KKH | ATCAAGCAGAGAAAGCCAG | 48 | E51SaCas9KKH | GAGTAACAGTCTGAGTAGG |
| | 9 | TCG | | 22 | AGC |
| 35 | E51SaCas9KKH | ATCAAGCAGAGAAAGCCAG | 53 | E51SaCas9KKH | TCAACGAGATGATCATCAA |
| | 9 | TCG | | 27 | GCA |
| 35 | E51SaCas9KKH | ATCAAGCAGAGAAAGCCAG | 54 | E51SaCas9KKH | GTGACCTTGAGGATATCAA |
| | 9 | TCG | | 28 | CGA |
| 35 | E51SaCas9KKH | ATCAAGCAGAGAAAGCCAG | 55 | E51SaCas9KKH | TGGGTGACCTTGAGGATAT |
| | 9 | TCG | | 29 | CAA |
| 35 | E51SaCas9KKH | ATCAAGCAGAGAAAGCCAG | 56 | E51SaCas9KKH | GAGGGTGATGGTGGGTGAC |
| | 9 | TCG | | 30 | CTT |
| 35 | E51SaCas9KKH | ATCAAGCAGAGAAAGCCAG | 57 | E51SaCas9KKH | TATAAAATCACAGAGGGTG |
| | 9 | TCG | | 31 | ATG |
| 35 | E51SaCas9KKH | ATCAAGCAGAGAAAGCCAG | 58 | E51SaCas9KKH | AAGTTATAAAATCACAGAG |
| | 9 | TCG | | 32 | GGT |
| 35 | E51SaCas9KKH | ATCAAGCAGAGAAAGCCAG | 59 | E51SaCas9KKH | ATCAAGTTATAAAATCACA |
| | 9 | TCG | | 33 | GAG |
| 35 | E51SaCas9KKH | ATCAAGCAGAGAAAGCCAG | 60 | E51SaCas9KKH | TTGATCAAGTTATAAAATC |
| | 9 | TCG | | 34 | ACA |
| 35 | E51SaCas9KKH | ATCAAGCAGAGAAAGCCAG | 61 | E51SaCas9KKH | CTTTCTCTGCTTGATCAAG |
| | 9 | TCG | | 35 | TTA |
| 35 | E51SaCas9KKH | ATCAAGCAGAGAAAGCCAG | 62 | E51SaCas9KKH | CCGACTGGCTTTCTCTGCT |
| | 9 | TCG | | 36 | TGA |
| 35 | E51SaCas9KKH | ATCAAGCAGAGAAAGCCAG | 63 | E51SaCas9KKH | ACTTACCGACTGGCTTTCT |
| | 9 | TCG | | 37 | CTG |
| 35 | E51SaCas9KKH | ATCAAGCAGAGAAAGCCAG | 64 | E51SaCas9KKH | GATGTTGGAGGTACCTGCT |
| | 9 | TCG | | 38 | CTG |
| 35 | E51SaCas9KKH | ATCAAGCAGAGAAAGCCAG | 65 | E51SaCas9KKH | AAATGCCATCTTCCTTGAT |
| | 9 | TCG | | 39 | GTT |
| 35 | E51SaCas9KKH | ATCAAGCAGAGAAAGCCAG | 66 | E51SaCas9KKH | CCAAACTAGAAATGCCATC |
| | 9 | TCG | | 40 | TTC |
| 35 | E51SaCas9KKH | ATCAAGCAGAGAAAGCCAG | 67 | E51SaCas9KKH | AGGAAACTGCCATCTCCAA |
| | 9 | TCG | | 41 | ACT |
| 35 | E51SaCas9KKH | ATCAAGCAGAGAAAGCCAG | 68 | E51SaCas9KKH | CTGTTACTCTGGTGACACA |
| | 9 | TCG | | 42 | ACC |
| 35 | E51SaCas9KKH | ATCAAGCAGAGAAAGCCAG | 69 | E51SaCas9KKH | TAGCTCCTACTCAGACTGT |
| | 9 | TCG | | 43 | TAC |
| 36 | E51SaCas9KKH | CAGTCGGTAAGTTCTGTCC | 37 | E51SaCas9KKH | GTAAGTTCTGTCCAAGCCC |
| | 10 | AAG | | 11 | GGT |
| 36 | E51SaCas9KKH | CAGTCGGTAAGTTCTGTCC | 38 | E51SaCas9KKH | AGCCCGGTTGAAATCTGCC |
| | 10 | AAG | | 12 | AGA |
| 36 | E51SaCas9KKH | CAGTCGGTAAGTTCTGTCC | 39 | E51SaCas9KKH | AGCAGGTACCTCCAACATC |
| | 10 | AAG | | 13 | AAG |
| 36 | E51SaCas9KKH | CAGTCGGTAAGTTCTGTCC | 40 | E51SaCas9KKH | CAACATCAAGGAAGATGGC |
| | 10 | AAG | | 14 | ATT |
| 36 | E51SaCas9KKH | CAGTCGGTAAGTTCTGTCC | 41 | E51SaCas9KKH | AGGAAGATGGCATTTCTAG |
| | 10 | AAG | | 15 | TTT |
| 36 | E51SaCas9KKH | CAGTCGGTAAGTTCTGTCC | 42 | E51SaCas9KKH | ATGGCATTTCTAGTTTGGA |
| | 10 | AAG | | 16 | GAT |
| 36 | E51SaCas9KKH | CAGTCGGTAAGTTCTGTCC | 43 | E51SaCas9KKH | CTAGTTTGGAGATGGCAGT |
| | 10 | AAG | | 17 | TTC |
| 36 | E51SaCas9KKH | CAGTCGGTAAGTTCTGTCC | 44 | E51SaCas9KKH | GATGGCAGTTTCCTTAGTA |
| | 10 | AAG | | 18 | ACC |
| 36 | E51SaCas9KKH | CAGTCGGTAAGTTCTGTCC | 45 | E51SaCas9KKH | TAGTAACCACAGGTTGTGT |
| | 10 | AAG | | 19 | CAC |
| 36 | E51SaCas9KKH | CAGTCGGTAAGTTCTGTCC | 46 | E51SaCas9KKH | CCACAGGTTGTGTCACCAG |
| | 10 | AAG | | 20 | AGT |
| 36 | E51SaCas9KKH | CAGTCGGTAAGTTCTGTCC | 47 | E51SaCas9KKH | GTTGTGTCACCAGAGTAAC |
| | 10 | AAG | | 21 | AGT |
| 36 | E51SaCas9KKH | CAGTCGGTAAGTTCTGTCC | 48 | E51SaCas9KKH | GAGTAACAGTCTGAGTAGG |
| | 10 | AAG | | 22 | AGC |
| 36 | E51SaCas9KKH | CAGTCGGTAAGTTCTGTCC | 53 | E51SaCas9KKH | TCAACGAGATGATCATCAA |
| | 10 | AAG | | 27 | GCA |
| 36 | E51SaCas9KKH | CAGTCGGTAAGTTCTGTCC | 54 | E51SaCas9KKH | GTGACCTTGAGGATATCAA |
| | 10 | AAG | | 28 | CGA |
| 36 | E51SaCas9KKH | CAGTCGGTAAGTTCTGTCC | 55 | E51SaCas9KKH | TGGGTGACCTTGAGGATAT |
| | 10 | AAG | | 29 | CAA |
| 36 | E51SaCas9KKH | CAGTCGGTAAGTTCTGTCC | 56 | E51SaCas9KKH | GAGGGTGATGGTGGGTGAC |
| | 10 | AAG | | 30 | CTT |
| 36 | E51SaCas9KKH | CAGTCGGTAAGTTCTGTCC | 57 | E51SaCas9KKH | TATAAAATCACAGAGGGTG |
| | 10 | AAG | | 31 | ATG |
| 36 | E51SaCas9KKH | CAGTCGGTAAGTTCTGTCC | 58 | E51SaCas9KKH | AAGTTATAAAATCACAGAG |
| | 10 | AAG | | 32 | GGT |
| 36 | E51SaCas9KKH | CAGTCGGTAAGTTCTGTCC | 59 | E51SaCas9KKH | ATCAAGTTATAAAATCACA |
| | 10 | AAG | | 33 | GAG |
| 36 | E51SaCas9KKH | CAGTCGGTAAGTTCTGTCC | 60 | E51SaCas9KKH | TTGATCAAGTTATAAAATC |
| | 10 | AAG | | 34 | ACA |
| 36 | E51SaCas9KKH | CAGTCGGTAAGTTCTGTCC | 61 | E51SaCas9KKH | CTTTCTCTGCTTGATCAAG |
| | 10 | AAG | | 35 | TTA |
| 36 | E51SaCas9KKH | CAGTCGGTAAGTTCTGTCC | 62 | E51SaCas9KKH | CCGACTGGCTTTCTCTGCT |
| | 10 | AAG | | 36 | TGA |
| 36 | E51SaCas9KKH | CAGTCGGTAAGTTCTGTCC | 63 | E51SaCas9KKH | ACTTACCGACTGGCTTTCT |
| | 10 | AAG | | 37 | CTG |
| 36 | E51SaCas9KKH | CAGTCGGTAAGTTCTGTCC | 64 | E51SaCas9KKH | GATGTTGGAGGTACCTGCT |
| | 10 | AAG | | 38 | CTG |
| 36 | E51SaCas9KKH | CAGTCGGTAAGTTCTGTCC | 65 | E51SaCas9KKH | AAATGCCATCTTCCTTGAT |
| | 10 | AAG | | 39 | GTT |
| 36 | E51SaCas9KKH | CAGTCGGTAAGTTCTGTCC | 66 | E51SaCas9KKH | CCAAACTAGAAATGCCATC |
| | 10 | AAG | | 40 | TTC |
| 36 | E51SaCas9KKH | CAGTCGGTAAGTTCTGTCC | 67 | E51SaCas9KKH | AGGAAACTGCCATCTCCAA |
| | 10 | AAG | | 41 | ACT |
| 36 | E51SaCas9KKH | CAGTCGGTAAGTTCTGTCC | 68 | E51SaCas9KKH | CTGTTACTCTGGTGACACA |
| | 10 | AAG | | 42 | ACC |
| 36 | E51SaCas9KKH | CAGTCGGTAAGTTCTGTCC | 69 | E51SaCas9KKH | TAGCTCCTACTCAGACTGT |
| | 10 | AAG | | 43 | TAC |
| 37 | E51SaCas9KKH | GTAAGTTCTGTCCAAGCCC | 38 | E51SaCas9KKH | AGCCCGGTTGAAATCTGCC |
| | 11 | GGT | | 12 | AGA |
| 37 | E51SaCas9KKH | GTAAGTTCTGTCCAAGCCC | 39 | E51SaCas9KKH | AGCAGGTACCTCCAACATC |
| | 11 | GGT | | 13 | AAG |
| 37 | E51SaCas9KKH | GTAAGTTCTGTCCAAGCCC | 40 | E51SaCas9KKH | CAACATCAAGGAAGATGGC |
| | 11 | GGT | | 14 | ATT |
| 37 | E51SaCas9KKH | GTAAGTTCTGTCCAAGCCC | 41 | E51SaCas9KKH | AGGAAGATGGCATTTCTAG |
| | 11 | GGT | | 15 | TTT |
| 37 | E51SaCas9KKH | GTAAGTTCTGTCCAAGCCC | 42 | E51SaCas9KKH | ATGGCATTTCTAGTTTGGA |
| | 11 | GGT | | 16 | GAT |
| 37 | E51SaCas9KKH | GTAAGTTCTGTCCAAGCCC | 43 | E51SaCas9KKH | CTAGTTTGGAGATGGCAGT |
| | 11 | GGT | | 17 | TTC |
| 37 | E51SaCas9KKH | GTAAGTTCTGTCCAAGCCC | 44 | E51SaCas9KKH | GATGGCAGTTTCCTTAGTA |
| | 11 | GGT | | 18 | ACC |
| 37 | E51SaCas9KKH | GTAAGTTCTGTCCAAGCCC | 45 | E51SaCas9KKH | TAGTAACCACAGGTTGTGT |
| | 11 | GGT | | 19 | CAC |
| 37 | E51SaCas9KKH | GTAAGTTCTGTCCAAGCCC | 46 | E51SaCas9KKH | CCACAGGTTGTGTCACCAG |
| | 11 | GGT | | 20 | AGT |
| 37 | E51SaCas9KKH | GTAAGTTCTGTCCAAGCCC | 47 | E51SaCas9KKH | GTTGTGTCACCAGAGTAAC |
| | 11 | GGT | | 21 | AGT |
| 37 | E51SaCas9KKH | GTAAGTTCTGTCCAAGCCC | 48 | E51SaCas9KKH | GAGTAACAGTCTGAGTAGG |
| | 11 | GGT | | 22 | AGC |
| 37 | E51SaCas9KKH | GTAAGTTCTGTCCAAGCCC | 53 | E51SaCas9KKH | TCAACGAGATGATCATCAA |
| | 11 | GGT | | 27 | GCA |
| 37 | E51SaCas9KKH | GTAAGTTCTGTCCAAGCCC | 54 | E51SaCas9KKH | GTGACCTTGAGGATATCAA |
| | 11 | GGT | | 28 | CGA |
| 37 | E51SaCas9KKH | GTAAGTTCTGTCCAAGCCC | 55 | E51SaCas9KKH | TGGGTGACCTTGAGGATAT |
| | 11 | GGT | | 29 | CAA |
| 37 | E51SaCas9KKH | GTAAGTTCTGTCCAAGCCC | 56 | E51SaCas9KKH | GAGGGTGATGGTGGGTGAC |
| | 11 | GGT | | 30 | CTT |
| 37 | E51SaCas9KKH | GTAAGTTCTGTCCAAGCCC | 57 | E51SaCas9KKH | TATAAAATCACAGAGGGTG |
| | 11 | GGT | | 31 | ATG |
| 37 | E51SaCas9KKH | GTAAGTTCTGTCCAAGCCC | 58 | E51SaCas9KKH | AAGTTATAAAATCACAGAG |
| | 11 | GGT | | 32 | GGT |
| 37 | E51SaCas9KKH | GTAAGTTCTGTCCAAGCCC | 59 | E51SaCas9KKH | ATCAAGTTATAAAATCACA |
| | 11 | GGT | | 33 | GAG |
| 37 | E51SaCas9KKH | GTAAGTTCTGTCCAAGCCC | 60 | E51SaCas9KKH | TTGATCAAGTTATAAAATC |
| | 11 | GGT | | 34 | ACA |
| 37 | E51SaCas9KKH | GTAAGTTCTGTCCAAGCCC | 61 | E51SaCas9KKH | CTTTCTCTGCTTGATCAAG |
| | 11 | GGT | | 35 | TTA |
| 37 | E51SaCas9KKH | GTAAGTTCTGTCCAAGCCC | 62 | E51SaCas9KKH | CCGACTGGCTTTCTCTGCT |
| | 11 | GGT | | 36 | TGA |
| 37 | E51SaCas9KKH | GTAAGTTCTGTCCAAGCCC | 63 | E51SaCas9KKH | ACTTACCGACTGGCTTTCT |
| | 11 | GGT | | 37 | CTG |
| 37 | E51SaCas9KKH | GTAAGTTCTGTCCAAGCCC | 64 | E51SaCas9KKH | GATGTTGGAGGTACCTGCT |
| | 11 | GGT | | 38 | CTG |
| 37 | E51SaCas9KKH | GTAAGTTCTGTCCAAGCCC | 65 | E51SaCas9KKH | AAATGCCATCTTCCTTGAT |
| | 11 | GGT | | 39 | GTT |
| 37 | E51SaCas9KKH | GTAAGTTCTGTCCAAGCCC | 66 | E51SaCas9KKH | CCAAACTAGAAATGCCATC |
| | 11 | GGT | | 40 | TTC |
| 37 | E51SaCas9KKH | GTAAGTTCTGTCCAAGCCC | 67 | E51SaCas9KKH | AGGAAACTGCCATCTCCAA |
| | 11 | GGT | | 41 | ACT |
| 37 | E51SaCas9KKH | GTAAGTTCTGTCCAAGCCC | 68 | E51SaCas9KKH | CTGTTACTCTGGTGACACA |
| | 11 | GGT | | 42 | ACC |
| 37 | E51SaCas9KKH | GTAAGTTCTGTCCAAGCCC | 69 | E51SaCas9KKH | TAGCTCCTACTCAGACTGT |
| | 11 | GGT | | 43 | TAC |
| 38 | E51SaCas9KKH | AGCCCGGTTGAAATCTGCC | 39 | E51SaCas9KKH | AGCAGGTACCTCCAACATC |
| | 12 | AGA | | 13 | AAG |
| 38 | E51SaCas9KKH | AGCCCGGTTGAAATCTGCC | 40 | E51SaCas9KKH | CAACATCAAGGAAGATGGC |
| | 12 | AGA | | 14 | ATT |
| 38 | E51SaCas9KKH | AGCCCGGTTGAAATCTGCC | 41 | E51SaCas9KKH | AGGAAGATGGCATTTCTAG |
| | 12 | AGA | | 15 | TTT |
| 38 | E51SaCas9KKH | AGCCCGGTTGAAATCTGCC | 42 | E51SaCas9KKH | ATGGCATTTCTAGTTTGGA |
| | 12 | AGA | | 16 | GAT |
| 38 | E51SaCas9KKH | AGCCCGGTTGAAATCTGCC | 43 | E51SaCas9KKH | CTAGTTTGGAGATGGCAGT |
| | 12 | AGA | | 17 | TTC |
| 38 | E51SaCas9KKH | AGCCCGGTTGAAATCTGCC | 44 | E51SaCas9KKH | GATGGCAGTTTCCTTAGTA |
| | 12 | AGA | | 18 | ACC |
| 38 | E51SaCas9KKH | AGCCCGGTTGAAATCTGCC | 45 | E51SaCas9KKH | TAGTAACCACAGGTTGTGT |
| | 12 | AGA | | 19 | CAC |
| 38 | E51SaCas9KKH | AGCCCGGTTGAAATCTGCC | 46 | E51SaCas9KKH | CCACAGGTTGTGTCACCAG |
| | 12 | AGA | | 20 | AGT |
| 38 | E51SaCas9KKH | AGCCCGGTTGAAATCTGCC | 47 | E51SaCas9KKH | GTTGTGTCACCAGAGTAAC |
| | 12 | AGA | | 21 | AGT |
| 38 | E51SaCas9KKH | AGCCCGGTTGAAATCTGCC | 48 | E51SaCas9KKH | GAGTAACAGTCTGAGTAGG |
| | 12 | AGA | | 22 | AGC |
| 38 | E51SaCas9KKH | AGCCCGGTTGAAATCTGCC | 53 | E51SaCas9KKH | TCAACGAGATGATCATCAA |
| | 12 | AGA | | 27 | GCA |
| 38 | E51SaCas9KKH | AGCCCGGTTGAAATCTGCC | 54 | E51SaCas9KKH | GTGACCTTGAGGATATCAA |
| | 12 | AGA | | 28 | CGA |
| 38 | E51SaCas9KKH | AGCCCGGTTGAAATCTGCC | 55 | E51SaCas9KKH | TGGGTGACCTTGAGGATAT |
| | 12 | AGA | | 29 | CAA |
| 38 | E51SaCas9KKH | AGCCCGGTTGAAATCTGCC | 56 | E51SaCas9KKH | GAGGGTGATGGTGGGTGAC |
| | 12 | AGA | | 30 | CTT |
| 38 | E51SaCas9KKH | AGCCCGGTTGAAATCTGCC | 57 | E51SaCas9KKH | TATAAAATCACAGAGGGTG |
| | 12 | AGA | | 31 | ATG |
| 38 | E51SaCas9KKH | AGCCCGGTTGAAATCTGCC | 58 | E51SaCas9KKH | AAGTTATAAAATCACAGAG |
| | 12 | AGA | | 32 | GGT |
| 38 | E51SaCas9KKH | AGCCCGGTTGAAATCTGCC | 59 | E51SaCas9KKH | ATCAAGTTATAAAATCACA |
| | 12 | AGA | | 33 | GAG |
| 38 | E51SaCas9KKH | AGCCCGGTTGAAATCTGCC | 60 | E51SaCas9KKH | TTGATCAAGTTATAAAATC |
| | 12 | AGA | | 34 | ACA |
| 38 | E51SaCas9KKH | AGCCCGGTTGAAATCTGCC | 61 | E51SaCas9KKH | CTTTCTCTGCTTGATCAAG |
| | 12 | AGA | | 35 | TTA |
| 38 | E51SaCas9KKH | AGCCCGGTTGAAATCTGCC | 62 | E51SaCas9KKH | CCGACTGGCTTTCTCTGCT |
| | 12 | AGA | | 36 | TGA |
| 38 | E51SaCas9KKH | AGCCCGGTTGAAATCTGCC | 63 | E51SaCas9KKH | ACTTACCGACTGGCTTTCT |
| | 12 | AGA | | 37 | CTG |
| 38 | E51SaCas9KKH | AGCCCGGTTGAAATCTGCC | 64 | E51SaCas9KKH | GATGTTGGAGGTACCTGCT |
| | 12 | AGA | | 38 | CTG |
| 38 | E51SaCas9KKH | AGCCCGGTTGAAATCTGCC | 65 | E51SaCas9KKH | AAATGCCATCTTCCTTGAT |
| | 12 | AGA | | 39 | GTT |
| 38 | E51SaCas9KKH | AGCCCGGTTGAAATCTGCC | 66 | E51SaCas9KKH | CCAAACTAGAAATGCCATC |
| | 12 | AGA | | 40 | TTC |
| 38 | E51SaCas9KKH | AGCCCGGTTGAAATCTGCC | 67 | E51SaCas9KKH | AGGAAACTGCCATCTCCAA |
| | 12 | AGA | | 41 | ACT |
| 38 | E51SaCas9KKH | AGCCCGGTTGAAATCTGCC | 68 | E51SaCas9KKH | CTGTTACTCTGGTGACACA |
| | 12 | AGA | | 42 | ACC |
| 38 | E51SaCas9KKH | AGCCCGGTTGAAATCTGCC | 69 | E51SaCas9KKH | TAGCTCCTACTCAGACTGT |
| | 12 | AGA | | 43 | TAC |
| 39 | E51SaCas9KKH | AGCAGGTACCTCCAACATC | 40 | E51SaCas9KKH | CAACATCAAGGAAGATGGC |
| | 13 | AAG | | 14 | ATT |
| 39 | E51SaCas9KKH | AGCAGGTACCTCCAACATC | 41 | E51SaCas9KKH | AGGAAGATGGCATTTCTAG |
| | 13 | AAG | | 15 | TTT |
| 39 | E51SaCas9KKH | AGCAGGTACCTCCAACATC | 42 | E51SaCas9KKH | ATGGCATTTCTAGTTTGGA |
| | 13 | AAG | | 16 | GAT |
| 39 | E51SaCas9KKH | AGCAGGTACCTCCAACATC | 43 | E51SaCas9KKH | CTAGTTTGGAGATGGCAGT |
| | 13 | AAG | | 17 | TTC |
| 39 | E51SaCas9KKH | AGCAGGTACCTCCAACATC | 44 | E51SaCas9KKH | GATGGCAGTTTCCTTAGTA |
| | 13 | AAG | | 18 | ACC |
| 39 | E51SaCas9KKH | AGCAGGTACCTCCAACATC | 45 | E51SaCas9KKH | TAGTAACCACAGGTTGTGT |
| | 13 | AAG | | 19 | CAC |
| 39 | E51SaCas9KKH | AGCAGGTACCTCCAACATC | 46 | E51SaCas9KKH | CCACAGGTTGTGTCACCAG |
| | 13 | AAG | | 20 | AGT |
| 39 | E51SaCas9KKH | AGCAGGTACCTCCAACATC | 47 | E51SaCas9KKH | GTTGTGTCACCAGAGTAAC |
| | 13 | AAG | | 21 | AGT |
| 39 | E51SaCas9KKH | AGCAGGTACCTCCAACATC | 48 | E51SaCas9KKH | GAGTAACAGTCTGAGTAGG |
| | 13 | AAG | | 22 | AGC |
| 39 | E51SaCas9KKH | AGCAGGTACCTCCAACATC | 53 | E51SaCas9KKH | TCAACGAGATGATCATCAA |
| | 13 | AAG | | 27 | GCA |
| 39 | E51SaCas9KKH | AGCAGGTACCTCCAACATC | 54 | E51SaCas9KKH | GTGACCTTGAGGATATCAA |
| | 13 | AAG | | 28 | CGA |
| 39 | E51SaCas9KKH | AGCAGGTACCTCCAACATC | 55 | E51SaCas9KKH | TGGGTGACCTTGAGGATAT |
| | 13 | AAG | | 29 | CAA |
| 39 | E51SaCas9KKH | AGCAGGTACCTCCAACATC | 56 | E51SaCas9KKH | GAGGGTGATGGTGGGTGAC |
| | 13 | AAG | | 30 | CTT |
| 39 | E51SaCas9KKH | AGCAGGTACCTCCAACATC | 57 | E51SaCas9KKH | TATAAAATCACAGAGGGTG |
| | 13 | AAG | | 31 | ATG |
| 39 | E51SaCas9KKH | AGCAGGTACCTCCAACATC | 58 | E51SaCas9KKH | AAGTTATAAAATCACAGAG |
| | 13 | AAG | | 32 | GGT |
| 39 | E51SaCas9KKH | AGCAGGTACCTCCAACATC | 59 | E51SaCas9KKH | ATCAAGTTATAAAATCACA |
| | 13 | AAG | | 33 | GAG |
| 39 | E51SaCas9KKH | AGCAGGTACCTCCAACATC | 60 | E51SaCas9KKH | TTGATCAAGTTATAAAATC |
| | 13 | AAG | | 34 | ACA |
| 39 | E51SaCas9KKH | AGCAGGTACCTCCAACATC | 61 | E51SaCas9KKH | CTTTCTCTGCTTGATCAAG |
| | 13 | AAG | | 35 | TTA |
| 39 | E51SaCas9KKH | AGCAGGTACCTCCAACATC | 62 | E51SaCas9KKH | CCGACTGGCTTTCTCTGCT |
| | 13 | AAG | | 36 | TGA |
| 39 | E51SaCas9KKH | AGCAGGTACCTCCAACATC | 63 | E51SaCas9KKH | ACTTACCGACTGGCTTTCT |
| | 13 | AAG | | 37 | CTG |
| 39 | E51SaCas9KKH | AGCAGGTACCTCCAACATC | 64 | E51SaCas9KKH | GATGTTGGAGGTACCTGCT |
| | 13 | AAG | | 38 | CTG |
| 39 | E51SaCas9KKH | AGCAGGTACCTCCAACATC | 65 | E51SaCas9KKH | AAATGCCATCTTCCTTGAT |
| | 13 | AAG | | 39 | GTT |
| 39 | E51SaCas9KKH | AGCAGGTACCTCCAACATC | 66 | E51SaCas9KKH | CCAAACTAGAAATGCCATC |
| | 13 | AAG | | 40 | TTC |
| 39 | E51SaCas9KKH | AGCAGGTACCTCCAACATC | 67 | E51SaCas9KKH | AGGAAACTGCCATCTCCAA |
| | 13 | AAG | | 41 | ACT |
| 39 | E51SaCas9KKH | AGCAGGTACCTCCAACATC | 68 | E51SaCas9KKH | CTGTTACTCTGGTGACACA |
| | 13 | AAG | | 42 | ACC |
| 39 | E51SaCas9KKH | AGCAGGTACCTCCAACATC | 69 | E51SaCas9KKH | TAGCTCCTACTCAGACTGT |
| | 13 | AAG | | 43 | TAC |
| 40 | E51SaCas9KKH | CAACATCAAGGAAGATGGC | 41 | E51SaCas9KKH | AGGAAGATGGCATTTCTAG |
| | 14 | ATT | | 15 | TTT |
| 40 | E51SaCas9KKH | CAACATCAAGGAAGATGGC | 42 | E51SaCas9KKH | ATGGCATTTCTAGTTTGGA |
| | 14 | ATT | | 16 | GAT |
| 40 | E51SaCas9KKH | CAACATCAAGGAAGATGGC | 43 | E51SaCas9KKH | CTAGTTTGGAGATGGCAGT |
| | 14 | ATT | | 17 | TTC |
| 40 | E51SaCas9KKH | CAACATCAAGGAAGATGGC | 44 | E51SaCas9KKH | GATGGCAGTTTCCTTAGTA |
| | 14 | ATT | | 18 | ACC |
| 40 | E51SaCas9KKH | CAACATCAAGGAAGATGGC | 45 | E51SaCas9KKH | TAGTAACCACAGGTTGTGT |
| | 14 | ATT | | 19 | CAC |
| 40 | E51SaCas9KKH | CAACATCAAGGAAGATGGC | 46 | E51SaCas9KKH | CCACAGGTTGTGTCACCAG |
| | 14 | ATT | | 20 | AGT |
| 40 | E51SaCas9KKH | CAACATCAAGGAAGATGGC | 47 | E51SaCas9KKH | GTTGTGTCACCAGAGTAAC |
| | 14 | ATT | | 21 | AGT |
| 40 | E51SaCas9KKH | CAACATCAAGGAAGATGGC | 48 | E51SaCas9KKH | GAGTAACAGTCTGAGTAGG |
| | 14 | ATT | | 22 | AGC |
| 40 | E51SaCas9KKH | CAACATCAAGGAAGATGGC | 53 | E51SaCas9KKH | TCAACGAGATGATCATCAA |
| | 14 | ATT | | 27 | GCA |
| 40 | E51SaCas9KKH | CAACATCAAGGAAGATGGC | 54 | E51SaCas9KKH | GTGACCTTGAGGATATCAA |
| | 14 | ATT | | 28 | CGA |
| 40 | E51SaCas9KKH | CAACATCAAGGAAGATGGC | 55 | E51SaCas9KKH | TGGGTGACCTTGAGGATAT |
| | 14 | ATT | | 29 | CAA |
| 40 | E51SaCas9KKH | CAACATCAAGGAAGATGGC | 56 | E51SaCas9KKH | GAGGGTGATGGTGGGTGAC |
| | 14 | ATT | | 30 | CTT |
| 40 | E51SaCas9KKH | CAACATCAAGGAAGATGGC | 57 | E51SaCas9KKH | TATAAAATCACAGAGGGTG |
| | 14 | ATT | | 31 | ATG |
| 40 | E51SaCas9KKH | CAACATCAAGGAAGATGGC | 58 | E51SaCas9KKH | AAGTTATAAAATCACAGAG |
| | 14 | ATT | | 32 | GGT |
| 40 | E51SaCas9KKH | CAACATCAAGGAAGATGGC | 59 | E51SaCas9KKH | ATCAAGTTATAAAATCACA |
| | 14 | ATT | | 33 | GAG |
| 40 | E51SaCas9KKH | CAACATCAAGGAAGATGGC | 60 | E51SaCas9KKH | TTGATCAAGTTATAAAATC |
| | 14 | ATT | | 34 | ACA |
| 40 | E51SaCas9KKH | CAACATCAAGGAAGATGGC | 61 | E51SaCas9KKH | CTTTCTCTGCTTGATCAAG |
| | 14 | ATT | | 35 | TTA |
| 40 | E51SaCas9KKH | CAACATCAAGGAAGATGGC | 62 | E51SaCas9KKH | CCGACTGGCTTTCTCTGCT |
| | 14 | ATT | | 36 | TGA |
| 40 | E51SaCas9KKH | CAACATCAAGGAAGATGGC | 63 | E51SaCas9KKH | ACTTACCGACTGGCTTTCT |
| | 14 | ATT | | 37 | CTG |
| 40 | E51SaCas9KKH | CAACATCAAGGAAGATGGC | 64 | E51SaCas9KKH | GATGTTGGAGGTACCTGCT |
| | 14 | ATT | | 38 | CTG |
| 40 | E51SaCas9KKH | CAACATCAAGGAAGATGGC | 65 | E51SaCas9KKH | AAATGCCATCTTCCTTGAT |
| | 14 | ATT | | 39 | GTT |
| 40 | E51SaCas9KKH | CAACATCAAGGAAGATGGC | 66 | E51SaCas9KKH | CCAAACTAGAAATGCCATC |
| | 14 | ATT | | 40 | TTC |
| 40 | E51SaCas9KKH | CAACATCAAGGAAGATGGC | 67 | E51SaCas9KKH | AGGAAACTGCCATCTCCAA |
| | 14 | ATT | | 41 | ACT |
| 40 | E51SaCas9KKH | CAACATCAAGGAAGATGGC | 68 | E51SaCas9KKH | CTGTTACTCTGGTGACACA |
| | 14 | ATT | | 42 | ACC |
| 40 | E51SaCas9KKH | CAACATCAAGGAAGATGGC | 69 | E51SaCas9KKH | TAGCTCCTACTCAGACTGT |
| | 14 | ATT | | 43 | TAC |
| 41 | E51SaCas9KKH | AGGAAGATGGCATTTCTAG | 42 | E51SaCas9KKH | ATGGCATTTCTAGTTTGGA |
| | 15 | TTT | | 16 | GAT |
| 41 | E51SaCas9KKH | AGGAAGATGGCATTTCTAG | 43 | E51SaCas9KKH | CTAGTTTGGAGATGGCAGT |
| | 15 | TTT | | 17 | TTC |
| 41 | E51SaCas9KKH | AGGAAGATGGCATTTCTAG | 44 | E51SaCas9KKH | GATGGCAGTTTCCTTAGTA |
| | 15 | TTT | | 18 | ACC |
| 41 | E51SaCas9KKH | AGGAAGATGGCATTTCTAG | 45 | E51SaCas9KKH | TAGTAACCACAGGTTGTGT |
| | 15 | TTT | | 19 | CAC |
| 41 | E51SaCas9KKH | AGGAAGATGGCATTTCTAG | 46 | E51SaCas9KKH | CCACAGGTTGTGTCACCAG |
| | 15 | TTT | | 20 | AGT |
| 41 | E51SaCas9KKH | AGGAAGATGGCATTTCTAG | 47 | E51SaCas9KKH | GTTGTGTCACCAGAGTAAC |
| | 15 | TTT | | 21 | AGT |
| 41 | E51SaCas9KKH | AGGAAGATGGCATTTCTAG | 48 | E51SaCas9KKH | GAGTAACAGTCTGAGTAGG |
| | 15 | TTT | | 22 | AGC |
| 41 | E51SaCas9KKH | AGGAAGATGGCATTTCTAG | 53 | E51SaCas9KKH | TCAACGAGATGATCATCAA |
| | 15 | TTT | | 27 | GCA |
| 41 | E51SaCas9KKH | AGGAAGATGGCATTTCTAG | 54 | E51SaCas9KKH | GTGACCTTGAGGATATCAA |
| | 15 | TTT | | 28 | CGA |
| 41 | E51SaCas9KKH | AGGAAGATGGCATTTCTAG | 55 | E51SaCas9KKH | TGGGTGACCTTGAGGATAT |
| | 15 | TTT | | 29 | CAA |
| 41 | E51SaCas9KKH | AGGAAGATGGCATTTCTAG | 56 | E51SaCas9KKH | GAGGGTGATGGTGGGTGAC |
| | 15 | TTT | | 30 | CTT |
| 41 | E51SaCas9KKH | AGGAAGATGGCATTTCTAG | 57 | E51SaCas9KKH | TATAAAATCACAGAGGGTG |
| | 15 | TTT | | 31 | ATG |
| 41 | E51SaCas9KKH | AGGAAGATGGCATTTCTAG | 58 | E51SaCas9KKH | AAGTTATAAAATCACAGAG |
| | 15 | TTT | | 32 | GGT |
| 41 | E51SaCas9KKH | AGGAAGATGGCATTTCTAG | 59 | E51SaCas9KKH | ATCAAGTTATAAAATCACA |
| | 15 | TTT | | 33 | GAG |
| 41 | E51SaCas9KKH | AGGAAGATGGCATTTCTAG | 60 | E51SaCas9KKH | TTGATCAAGTTATAAAATC |
| | 15 | TTT | | 34 | ACA |
| 41 | E51SaCas9KKH | AGGAAGATGGCATTTCTAG | 61 | E51SaCas9KKH | CTTTCTCTGCTTGATCAAG |
| | 15 | TTT | | 35 | TTA |
| 41 | E51SaCas9KKH | AGGAAGATGGCATTTCTAG | 62 | E51SaCas9KKH | CCGACTGGCTTTCTCTGCT |
| | 15 | TTT | | 36 | TGA |
| 41 | E51SaCas9KKH | AGGAAGATGGCATTTCTAG | 63 | E51SaCas9KKH | ACTTACCGACTGGCTTTCT |
| | 15 | TTT | | 37 | CTG |
| 41 | E51SaCas9KKH | AGGAAGATGGCATTTCTAG | 64 | E51SaCas9KKH | GATGTTGGAGGTACCTGCT |
| | 15 | TTT | | 38 | CTG |
| 41 | E51SaCas9KKH | AGGAAGATGGCATTTCTAG | 65 | E51SaCas9KKH | AAATGCCATCTTCCTTGAT |
| | 15 | TTT | | 39 | GTT |
| 41 | E51SaCas9KKH | AGGAAGATGGCATTTCTAG | 66 | E51SaCas9KKH | CCAAACTAGAAATGCCATC |
| | 15 | TTT | | 40 | TTC |
| 41 | E51SaCas9KKH | AGGAAGATGGCATTTCTAG | 67 | E51SaCas9KKH | AGGAAACTGCCATCTCCAA |
| | 15 | TTT | | 41 | ACT |
| 41 | E51SaCas9KKH | AGGAAGATGGCATTTCTAG | 68 | E51SaCas9KKH | CTGTTACTCTGGTGACACA |
| | 15 | TTT | | 42 | ACC |
| 41 | E51SaCas9KKH | AGGAAGATGGCATTTCTAG | 69 | E51SaCas9KKH | TAGCTCCTACTCAGACTGT |
| | 15 | TTT | | 43 | TAC |
| 42 | E51SaCas9KKH | ATGGCATTTCTAGTTTGGA | 43 | E51SaCas9KKH | CTAGTTTGGAGATGGCAGT |
| | 16 | GAT | | 17 | TTC |
| 42 | E51SaCas9KKH | ATGGCATTTCTAGTTTGGA | 44 | E51SaCas9KKH | GATGGCAGTTTCCTTAGTA |
| | 16 | GAT | | 18 | ACC |
| 42 | E51SaCas9KKH | ATGGCATTTCTAGTTTGGA | 45 | E51SaCas9KKH | TAGTAACCACAGGTTGTGT |
| | 16 | GAT | | 19 | CAC |
| 42 | E51SaCas9KKH | ATGGCATTTCTAGTTTGGA | 46 | E51SaCas9KKH | CCACAGGTTGTGTCACCAG |
| | 16 | GAT | | 20 | AGT |
| 42 | E51SaCas9KKH | ATGGCATTTCTAGTTTGGA | 47 | E51SaCas9KKH | GTTGTGTCACCAGAGTAAC |
| | 16 | GAT | | 21 | AGT |
| 42 | E51SaCas9KKH | ATGGCATTTCTAGTTTGGA | 48 | E51SaCas9KKH | GAGTAACAGTCTGAGTAGG |
| | 16 | GAT | | 22 | AGC |
| 42 | E51SaCas9KKH | ATGGCATTTCTAGTTTGGA | 53 | E51SaCas9KKH | TCAACGAGATGATCATCAA |
| | 16 | GAT | | 27 | GCA |
| 42 | E51SaCas9KKH | ATGGCATTTCTAGTTTGGA | 54 | E51SaCas9KKH | GTGACCTTGAGGATATCAA |
| | 16 | GAT | | 28 | CGA |
| 42 | E51SaCas9KKH | ATGGCATTTCTAGTTTGGA | 55 | E51SaCas9KKH | TGGGTGACCTTGAGGATAT |
| | 16 | GAT | | 29 | CAA |
| 42 | E51SaCas9KKH | ATGGCATTTCTAGTTTGGA | 56 | E51SaCas9KKH | GAGGGTGATGGTGGGTGAC |
| | 16 | GAT | | 30 | CTT |
| 42 | E51SaCas9KKH | ATGGCATTTCTAGTTTGGA | 57 | E51SaCas9KKH | TATAAAATCACAGAGGGTG |
| | 16 | GAT | | 31 | ATG |
| 42 | E51SaCas9KKH | ATGGCATTTCTAGTTTGGA | 58 | E51SaCas9KKH | AAGTTATAAAATCACAGAG |
| | 16 | GAT | | 32 | GGT |
| 42 | E51SaCas9KKH | ATGGCATTTCTAGTTTGGA | 59 | E51SaCas9KKH | ATCAAGTTATAAAATCACA |
| | 16 | GAT | | 33 | GAG |
| 42 | E51SaCas9KKH | ATGGCATTTCTAGTTTGGA | 60 | E51SaCas9KKH | TTGATCAAGTTATAAAATC |
| | 16 | GAT | | 34 | ACA |
| 42 | E51SaCas9KKH | ATGGCATTTCTAGTTTGGA | 61 | E51SaCas9KKH | CTTTCTCTGCTTGATCAAG |
| | 16 | GAT | | 35 | TTA |
| 42 | E51SaCas9KKH | ATGGCATTTCTAGTTTGGA | 62 | E51SaCas9KKH | CCGACTGGCTTTCTCTGCT |
| | 16 | GAT | | 36 | TGA |
| 42 | E51SaCas9KKH | ATGGCATTTCTAGTTTGGA | 63 | E51SaCas9KKH | ACTTACCGACTGGCTTTCT |
| | 16 | GAT | | 37 | CTG |
| 42 | E51SaCas9KKH | ATGGCATTTCTAGTTTGGA | 64 | E51SaCas9KKH | GATGTTGGAGGTACCTGCT |
| | 16 | GAT | | 38 | CTG |
| 42 | E51SaCas9KKH | ATGGCATTTCTAGTTTGGA | 65 | E51SaCas9KKH | AAATGCCATCTTCCTTGAT |
| | 16 | GAT | | 39 | GTT |
| 42 | E51SaCas9KKH | ATGGCATTTCTAGTTTGGA | 66 | E51SaCas9KKH | CCAAACTAGAAATGCCATC |
| | 16 | GAT | | 40 | TTC |
| 42 | E51SaCas9KKH | ATGGCATTTCTAGTTTGGA | 67 | E51SaCas9KKH | AGGAAACTGCCATCTCCAA |
| | 16 | GAT | | 41 | ACT |
| 42 | E51SaCas9KKH | ATGGCATTTCTAGTTTGGA | 68 | E51SaCas9KKH | CTGTTACTCTGGTGACACA |
| | 16 | GAT | | 42 | ACC |
| 42 | E51SaCas9KKH | ATGGCATTTCTAGTTTGGA | 69 | E51SaCas9KKH | TAGCTCCTACTCAGACTGT |
| | 16 | GAT | | 43 | TAC |
| 43 | E51SaCas9KKH | CTAGTTTGGAGATGGCAGT | 44 | E51SaCas9KKH | GATGGCAGTTTCCTTAGTA |
| | 17 | TTC | | 18 | ACC |
| 43 | E51SaCas9KKH | CTAGTTTGGAGATGGCAGT | 45 | E51SaCas9KKH | TAGTAACCACAGGTTGTGT |
| | 17 | TTC | | 19 | CAC |
| 43 | E51SaCas9KKH | CTAGTTTGGAGATGGCAGT | 46 | E51SaCas9KKH | CCACAGGTTGTGTCACCAG |
| | 17 | TTC | | 20 | AGT |
| 43 | E51SaCas9KKH | CTAGTTTGGAGATGGCAGT | 47 | E51SaCas9KKH | GTTGTGTCACCAGAGTAAC |
| | 17 | TTC | | 21 | AGT |
| 43 | E51SaCas9KKH | CTAGTTTGGAGATGGCAGT | 48 | E51SaCas9KKH | GAGTAACAGTCTGAGTAGG |
| | 17 | TTC | | 22 | AGC |
| 43 | E51SaCas9KKH | CTAGTTTGGAGATGGCAGT | 53 | E51SaCas9KKH | TCAACGAGATGATCATCAA |
| | 17 | TTC | | 27 | GCA |
| 43 | E51SaCas9KKH | CTAGTTTGGAGATGGCAGT | 54 | E51SaCas9KKH | GTGACCTTGAGGATATCAA |
| | 17 | TTC | | 28 | CGA |
| 43 | E51SaCas9KKH | CTAGTTTGGAGATGGCAGT | 55 | E51SaCas9KKH | TGGGTGACCTTGAGGATAT |
| | 17 | TTC | | 29 | CAA |
| 43 | E51SaCas9KKH | CTAGTTTGGAGATGGCAGT | 56 | E51SaCas9KKH | GAGGGTGATGGTGGGTGAC |
| | 17 | TTC | | 30 | CTT |
| 43 | E51SaCas9KKH | CTAGTTTGGAGATGGCAGT | 57 | E51SaCas9KKH | TATAAAATCACAGAGGGTG |
| | 17 | TTC | | 31 | ATG |
| 43 | E51SaCas9KKH | CTAGTTTGGAGATGGCAGT | 58 | E51SaCas9KKH | AAGTTATAAAATCACAGAG |
| | 17 | TTC | | 32 | GGT |
| 43 | E51SaCas9KKH | CTAGTTTGGAGATGGCAGT | 59 | E51SaCas9KKH | ATCAAGTTATAAAATCACA |
| | 17 | TTC | | 33 | GAG |
| 43 | E51SaCas9KKH | CTAGTTTGGAGATGGCAGT | 60 | E51SaCas9KKH | TTGATCAAGTTATAAAATC |
| | 17 | TTC | | 34 | ACA |
| 43 | E51SaCas9KKH | CTAGTTTGGAGATGGCAGT | 61 | E51SaCas9KKH | CTTTCTCTGCTTGATCAAG |
| | 17 | TTC | | 35 | TTA |
| 43 | E51SaCas9KKH | CTAGTTTGGAGATGGCAGT | 62 | E51SaCas9KKH | CCGACTGGCTTTCTCTGCT |
| | 17 | TTC | | 36 | TGA |
| 43 | E51SaCas9KKH | CTAGTTTGGAGATGGCAGT | 63 | E51SaCas9KKH | ACTTACCGACTGGCTTTCT |
| | 17 | TTC | | 37 | CTG |
| 43 | E51SaCas9KKH | CTAGTTTGGAGATGGCAGT | 64 | E51SaCas9KKH | GATGTTGGAGGTACCTGCT |
| | 17 | TTC | | 38 | CTG |
| 43 | E51SaCas9KKH | CTAGTTTGGAGATGGCAGT | 65 | E51SaCas9KKH | AAATGCCATCTTCCTTGAT |
| | 17 | TTC | | 39 | GTT |
| 43 | E51SaCas9KKH | CTAGTTTGGAGATGGCAGT | 66 | E51SaCas9KKH | CCAAACTAGAAATGCCATC |
| | 17 | TTC | | 40 | TTC |
| 43 | E51SaCas9KKH | CTAGTTTGGAGATGGCAGT | 67 | E51SaCas9KKH | AGGAAACTGCCATCTCCAA |
| | 17 | TTC | | 41 | ACT |
| 43 | E51SaCas9KKH | CTAGTTTGGAGATGGCAGT | 68 | E51SaCas9KKH | CTGTTACTCTGGTGACACA |
| | 17 | TTC | | 42 | ACC |
| 43 | E51SaCas9KKH | CTAGTTTGGAGATGGCAGT | 69 | E51SaCas9KKH | TAGCTCCTACTCAGACTGT |
| | 17 | TTC | | 43 | TAC |
| 44 | E51SaCas9KKH | GATGGCAGTTTCCTTAGTA | 45 | E51SaCas9KKH | TAGTAACCACAGGTTGTGT |
| | 18 | ACC | | 19 | CAC |
| 44 | E51SaCas9KKH | GATGGCAGTTTCCTTAGTA | 46 | E51SaCas9KKH | CCACAGGTTGTGTCACCAG |
| | 18 | ACC | | 20 | AGT |
| 44 | E51SaCas9KKH | GATGGCAGTTTCCTTAGTA | 47 | E51SaCas9KKH | GTTGTGTCACCAGAGTAAC |
| | 18 | ACC | | 21 | AGT |
| 44 | E51SaCas9KKH | GATGGCAGTTTCCTTAGTA | 48 | E51SaCas9KKH | GAGTAACAGTCTGAGTAGG |
| | 18 | ACC | | 22 | AGC |
| 44 | E51SaCas9KKH | GATGGCAGTTTCCTTAGTA | 53 | E51SaCas9KKH | TCAACGAGATGATCATCAA |
| | 18 | ACC | | 27 | GCA |
| 44 | E51SaCas9KKH | GATGGCAGTTTCCTTAGTA | 54 | E51SaCas9KKH | GTGACCTTGAGGATATCAA |
| | 18 | ACC | | 28 | CGA |
| 44 | E51SaCas9KKH | GATGGCAGTTTCCTTAGTA | 55 | E51SaCas9KKH | TGGGTGACCTTGAGGATAT |
| | 18 | ACC | | 29 | CAA |
| 44 | E51SaCas9KKH | GATGGCAGTTTCCTTAGTA | 56 | E51SaCas9KKH | GAGGGTGATGGTGGGTGAC |
| | 18 | ACC | | 30 | CTT |
| 44 | E51SaCas9KKH | GATGGCAGTTTCCTTAGTA | 57 | E51SaCas9KKH | TATAAAATCACAGAGGGTG |
| | 18 | ACC | | 31 | ATG |
| 44 | E51SaCas9KKH | GATGGCAGTTTCCTTAGTA | 58 | E51SaCas9KKH | AAGTTATAAAATCACAGAG |
| | 18 | ACC | | 32 | GGT |
| 44 | E51SaCas9KKH | GATGGCAGTTTCCTTAGTA | 59 | E51SaCas9KKH | ATCAAGTTATAAAATCACA |
| | 18 | ACC | | 33 | GAG |
| 44 | E51SaCas9KKH | GATGGCAGTTTCCTTAGTA | 60 | E51SaCas9KKH | TTGATCAAGTTATAAAATC |
| | 18 | ACC | | 34 | ACA |
| 44 | E51SaCas9KKH | GATGGCAGTTTCCTTAGTA | 61 | E51SaCas9KKH | CTTTCTCTGCTTGATCAAG |
| | 18 | ACC | | 35 | TTA |
| 44 | E51SaCas9KKH | GATGGCAGTTTCCTTAGTA | 62 | E51SaCas9KKH | CCGACTGGCTTTCTCTGCT |
| | 18 | ACC | | 36 | TGA |
| 44 | E51SaCas9KKH | GATGGCAGTTTCCTTAGTA | 63 | E51SaCas9KKH | ACTTACCGACTGGCTTTCT |
| | 18 | ACC | | 37 | CTG |
| 44 | E51SaCas9KKH | GATGGCAGTTTCCTTAGTA | 64 | E51SaCas9KKH | GATGTTGGAGGTACCTGCT |
| | 18 | ACC | | 38 | CTG |
| 44 | E51SaCas9KKH | GATGGCAGTTTCCTTAGTA | 65 | E51SaCas9KKH | AAATGCCATCTTCCTTGAT |
| | 18 | ACC | | 39 | GTT |
| 44 | E51SaCas9KKH | GATGGCAGTTTCCTTAGTA | 66 | E51SaCas9KKH | CCAAACTAGAAATGCCATC |
| | 18 | ACC | | 40 | TTC |
| 44 | E51SaCas9KKH | GATGGCAGTTTCCTTAGTA | 67 | E51SaCas9KKH | AGGAAACTGCCATCTCCAA |
| | 18 | ACC | | 41 | ACT |
| 44 | E51SaCas9KKH | GATGGCAGTTTCCTTAGTA | 68 | E51SaCas9KKH | CTGTTACTCTGGTGACACA |
| | 18 | ACC | | 42 | ACC |
| 44 | E51SaCas9KKH | GATGGCAGTTTCCTTAGTA | 69 | E51SaCas9KKH | TAGCTCCTACTCAGACTGT |
| | 18 | ACC | | 43 | TAC |
| 45 | E51SaCas9KKH | TAGTAACCACAGGTTGTGT | 46 | E51SaCas9KKH | CCACAGGTTGTGTCACCAG |
| | 19 | CAC | | 20 | AGT |
| 45 | E51SaCas9KKH | TAGTAACCACAGGTTGTGT | 47 | E51SaCas9KKH | GTTGTGTCACCAGAGTAAC |
| | 19 | CAC | | 21 | AGT |
| 45 | E51SaCas9KKH | TAGTAACCACAGGTTGTGT | 48 | E51SaCas9KKH | GAGTAACAGTCTGAGTAGG |
| | 19 | CAC | | 22 | AGC |
| 45 | E51SaCas9KKH | TAGTAACCACAGGTTGTGT | 53 | E51SaCas9KKH | TCAACGAGATGATCATCAA |
| | 19 | CAC | | 27 | GCA |
| 45 | E51SaCas9KKH | TAGTAACCACAGGTTGTGT | 54 | E51SaCas9KKH | GTGACCTTGAGGATATCAA |
| | 19 | CAC | | 28 | CGA |
| 45 | E51SaCas9KKH | TAGTAACCACAGGTTGTGT | 55 | E51SaCas9KKH | TGGGTGACCTTGAGGATAT |
| | 19 | CAC | | 29 | CAA |
| 45 | E51SaCas9KKH | TAGTAACCACAGGTTGTGT | 56 | E51SaCas9KKH | GAGGGTGATGGTGGGTGAC |
| | 19 | CAC | | 30 | CTT |
| 45 | E51SaCas9KKH | TAGTAACCACAGGTTGTGT | 57 | E51SaCas9KKH | TATAAAATCACAGAGGGTG |
| | 19 | CAC | | 31 | ATG |
| 45 | E51SaCas9KKH | TAGTAACCACAGGTTGTGT | 58 | E51SaCas9KKH | AAGTTATAAAATCACAGAG |
| | 19 | CAC | | 32 | GGT |
| 45 | E51SaCas9KKH | TAGTAACCACAGGTTGTGT | 59 | E51SaCas9KKH | ATCAAGTTATAAAATCACA |
| | 19 | CAC | | 33 | GAG |
| 45 | E51SaCas9KKH | TAGTAACCACAGGTTGTGT | 60 | E51SaCas9KKH | TTGATCAAGTTATAAAATC |
| | 19 | CAC | | 34 | ACA |
| 45 | E51SaCas9KKH | TAGTAACCACAGGTTGTGT | 61 | E51SaCas9KKH | CTTTCTCTGCTTGATCAAG |
| | 19 | CAC | | 35 | TTA |
| 45 | E51SaCas9KKH | TAGTAACCACAGGTTGTGT | 62 | E51SaCas9KKH | CCGACTGGCTTTCTCTGCT |
| | 19 | CAC | | 36 | TGA |
| 45 | E51SaCas9KKH | TAGTAACCACAGGTTGTGT | 63 | E51SaCas9KKH | ACTTACCGACTGGCTTTCT |
| | 19 | CAC | | 37 | CTG |
| 45 | E51SaCas9KKH | TAGTAACCACAGGTTGTGT | 64 | E51SaCas9KKH | GATGTTGGAGGTACCTGCT |
| | 19 | CAC | | 38 | CTG |
| 45 | E51SaCas9KKH | TAGTAACCACAGGTTGTGT | 65 | E51SaCas9KKH | AAATGCCATCTTCCTTGAT |
| | 19 | CAC | | 39 | GTT |
| 45 | E51SaCas9KKH | TAGTAACCACAGGTTGTGT | 66 | E51SaCas9KKH | CCAAACTAGAAATGCCATC |
| | 19 | CAC | | 40 | TTC |
| 45 | E51SaCas9KKH | TAGTAACCACAGGTTGTGT | 67 | E51SaCas9KKH | AGGAAACTGCCATCTCCAA |
| | 19 | CAC | | 41 | ACT |
| 45 | E51SaCas9KKH | TAGTAACCACAGGTTGTGT | 68 | E51SaCas9KKH | CTGTTACTCTGGTGACACA |
| | 19 | CAC | | 42 | ACC |
| 45 | E51SaCas9KKH | TAGTAACCACAGGTTGTGT | 69 | E51SaCas9KKH | TAGCTCCTACTCAGACTGT |
| | 19 | CAC | | 43 | TAC |
| 46 | E51SaCas9KKH | CCACAGGTTGTGTCACCAG | 47 | E51SaCas9KKH | GTTGTGTCACCAGAGTAAC |
| | 20 | AGT | | 21 | AGT |
| 46 | E51SaCas9KKH | CCACAGGTTGTGTCACCAG | 48 | E51SaCas9KKH | GAGTAACAGTCTGAGTAGG |
| | 20 | AGT | | 22 | AGC |
| 46 | E51SaCas9KKH | CCACAGGTTGTGTCACCAG | 53 | E51SaCas9KKH | TCAACGAGATGATCATCAA |
| | 20 | AGT | | 27 | GCA |
| 46 | E51SaCas9KKH | CCACAGGTTGTGTCACCAG | 54 | E51SaCas9KKH | GTGACCTTGAGGATATCAA |
| | 20 | AGT | | 28 | CGA |
| 46 | E51SaCas9KKH | CCACAGGTTGTGTCACCAG | 55 | E51SaCas9KKH | TGGGTGACCTTGAGGATAT |
| | 20 | AGT | | 29 | CAA |
| 46 | E51SaCas9KKH | CCACAGGTTGTGTCACCAG | 56 | E51SaCas9KKH | GAGGGTGATGGTGGGTGAC |
| | 20 | AGT | | 30 | CTT |
| 46 | E51SaCas9KKH | CCACAGGTTGTGTCACCAG | 57 | E51SaCas9KKH | TATAAAATCACAGAGGGTG |
| | 20 | AGT | | 31 | ATG |
| 46 | E51SaCas9KKH | CCACAGGTTGTGTCACCAG | 58 | E51SaCas9KKH | AAGTTATAAAATCACAGAG |
| | 20 | AGT | | 32 | GGT |
| 46 | E51SaCas9KKH | CCACAGGTTGTGTCACCAG | 59 | E51SaCas9KKH | ATCAAGTTATAAAATCACA |
| | 20 | AGT | | 33 | GAG |
| 46 | E51SaCas9KKH | CCACAGGTTGTGTCACCAG | 60 | E51SaCas9KKH | TTGATCAAGTTATAAAATC |
| | 20 | AGT | | 34 | ACA |
| 46 | E51SaCas9KKH | CCACAGGTTGTGTCACCAG | 61 | E51SaCas9KKH | CTTTCTCTGCTTGATCAAG |
| | 20 | AGT | | 35 | TTA |
| 46 | E51SaCas9KKH | CCACAGGTTGTGTCACCAG | 62 | E51SaCas9KKH | CCGACTGGCTTTCTCTGCT |
| | 20 | AGT | | 36 | TGA |
| 46 | E51SaCas9KKH | CCACAGGTTGTGTCACCAG | 63 | E51SaCas9KKH | ACTTACCGACTGGCTTTCT |
| | 20 | AGT | | 37 | CTG |
| 46 | E51SaCas9KKH | CCACAGGTTGTGTCACCAG | 64 | E51SaCas9KKH | GATGTTGGAGGTACCTGCT |
| | 20 | AGT | | 38 | CTG |
| 46 | E51SaCas9KKH | CCACAGGTTGTGTCACCAG | 65 | E51SaCas9KKH | AAATGCCATCTTCCTTGAT |
| | 20 | AGT | | 39 | GTT |
| 46 | E51SaCas9KKH | CCACAGGTTGTGTCACCAG | 66 | E51SaCas9KKH | CCAAACTAGAAATGCCATC |
| | 20 | AGT | | 40 | TTC |
| 46 | E51SaCas9KKH | CCACAGGTTGTGTCACCAG | 67 | E51SaCas9KKH | AGGAAACTGCCATCTCCAA |
| | 20 | AGT | | 41 | ACT |
| 46 | E51SaCas9KKH | CCACAGGTTGTGTCACCAG | 68 | E51SaCas9KKH | CTGTTACTCTGGTGACACA |
| | 20 | AGT | | 42 | ACC |
| 46 | E51SaCas9KKH | CCACAGGTTGTGTCACCAG | 69 | E51SaCas9KKH | TAGCTCCTACTCAGACTGT |
| | 20 | AGT | | 43 | TAC |
| 47 | E51SaCas9KKH | GTTGTGTCACCAGAGTAAC | 48 | E51SaCas9KKH | GAGTAACAGTCTGAGTAGG |
| | 21 | AGT | | 22 | AGC |
| 47 | E51SaCas9KKH | GTTGTGTCACCAGAGTAAC | 53 | E51SaCas9KKH | TCAACGAGATGATCATCAA |
| | 21 | AGT | | 27 | GCA |
| 47 | E51SaCas9KKH | GTTGTGTCACCAGAGTAAC | 54 | E51SaCas9KKH | GTGACCTTGAGGATATCAA |
| | 21 | AGT | | 28 | CGA |
| 47 | E51SaCas9KKH | GTTGTGTCACCAGAGTAAC | 55 | E51SaCas9KKH | TGGGTGACCTTGAGGATAT |
| | 21 | AGT | | 29 | CAA |
| 47 | E51SaCas9KKH | GTTGTGTCACCAGAGTAAC | 56 | E51SaCas9KKH | GAGGGTGATGGTGGGTGAC |
| | 21 | AGT | | 30 | CTT |
| 47 | E51SaCas9KKH | GTTGTGTCACCAGAGTAAC | 57 | E51SaCas9KKH | TATAAAATCACAGAGGGTG |
| | 21 | AGT | | 31 | ATG |
| 47 | E51SaCas9KKH | GTTGTGTCACCAGAGTAAC | 58 | E51SaCas9KKH | AAGTTATAAAATCACAGAG |
| | 21 | AGT | | 32 | GGT |
| 47 | E51SaCas9KKH | GTTGTGTCACCAGAGTAAC | 59 | E51SaCas9KKH | ATCAAGTTATAAAATCACA |
| | 21 | AGT | | 33 | GAG |
| 47 | E51SaCas9KKH | GTTGTGTCACCAGAGTAAC | 60 | E51SaCas9KKH | TTGATCAAGTTATAAAATC |
| | 21 | AGT | | 34 | ACA |
| 47 | E51SaCas9KKH | GTTGTGTCACCAGAGTAAC | 61 | E51SaCas9KKH | CTTTCTCTGCTTGATCAAG |
| | 21 | AGT | | 35 | TTA |
| 47 | E51SaCas9KKH | GTTGTGTCACCAGAGTAAC | 62 | E51SaCas9KKH | CCGACTGGCTTTCTCTGCT |
| | 21 | AGT | | 36 | TGA |
| 47 | E51SaCas9KKH | GTTGTGTCACCAGAGTAAC | 63 | E51SaCas9KKH | ACTTACCGACTGGCTTTCT |
| | 21 | AGT | | 37 | CTG |
| 47 | E51SaCas9KKH | GTTGTGTCACCAGAGTAAC | 64 | E51SaCas9KKH | GATGTTGGAGGTACCTGCT |
| | 21 | AGT | | 38 | CTG |
| 47 | E51SaCas9KKH | GTTGTGTCACCAGAGTAAC | 65 | E51SaCas9KKH | AAATGCCATCTTCCTTGAT |
| | 21 | AGT | | 39 | GTT |
| 47 | E51SaCas9KKH | GTTGTGTCACCAGAGTAAC | 66 | E51SaCas9KKH | CCAAACTAGAAATGCCATC |
| | 21 | AGT | | 40 | TTC |
| 47 | E51SaCas9KKH | GTTGTGTCACCAGAGTAAC | 67 | E51SaCas9KKH | AGGAAACTGCCATCTCCAA |
| | 21 | AGT | | 41 | ACT |
| 47 | E51SaCas9KKH | GTTGTGTCACCAGAGTAAC | 68 | E51SaCas9KKH | CTGTTACTCTGGTGACACA |
| | 21 | AGT | | 42 | ACC |
| 47 | E51SaCas9KKH | GTTGTGTCACCAGAGTAAC | 69 | E51SaCas9KKH | TAGCTCCTACTCAGACTGT |
| | 21 | AGT | | 43 | TAC |
| 48 | E51SaCas9KKH | GAGTAACAGTCTGAGTAGG | 53 | E51SaCas9KKH | TCAACGAGATGATCATCAA |
| | 22 | AGC | | 27 | GCA |
| 48 | E51SaCas9KKH | GAGTAACAGTCTGAGTAGG | 54 | E51SaCas9KKH | GTGACCTTGAGGATATCAA |
| | 22 | AGC | | 28 | CGA |
| 48 | E51SaCas9KKH | GAGTAACAGTCTGAGTAGG | 55 | E51SaCas9KKH | TGGGTGACCTTGAGGATAT |
| | 22 | AGC | | 29 | CAA |
| 48 | E51SaCas9KKH | GAGTAACAGTCTGAGTAGG | 56 | E51SaCas9KKH | GAGGGTGATGGTGGGTGAC |
| | 22 | AGC | | 30 | CTT |
| 48 | E51SaCas9KKH | GAGTAACAGTCTGAGTAGG | 57 | E51SaCas9KKH | TATAAAATCACAGAGGGTG |
| | 22 | AGC | | 31 | ATG |
| 48 | E51SaCas9KKH | GAGTAACAGTCTGAGTAGG | 58 | E51SaCas9KKH | AAGTTATAAAATCACAGAG |
| | 22 | AGC | | 32 | GGT |
| 48 | E51SaCas9KKH | GAGTAACAGTCTGAGTAGG | 59 | E51SaCas9KKH | ATCAAGTTATAAAATCACA |
| | 22 | AGC | | 33 | GAG |
| 48 | E51SaCas9KKH | GAGTAACAGTCTGAGTAGG | 60 | E51SaCas9KKH | TTGATCAAGTTATAAAATC |
| | 22 | AGC | | 34 | ACA |
| 48 | E51SaCas9KKH | GAGTAACAGTCTGAGTAGG | 61 | E51SaCas9KKH | CTTTCTCTGCTTGATCAAG |
| | 22 | AGC | | 35 | TTA |
| 48 | E51SaCas9KKH | GAGTAACAGTCTGAGTAGG | 62 | E51SaCas9KKH | CCGACTGGCTTTCTCTGCT |
| | 22 | AGC | | 36 | TGA |
| 48 | E51SaCas9KKH | GAGTAACAGTCTGAGTAGG | 63 | E51SaCas9KKH | ACTTACCGACTGGCTTTCT |
| | 22 | AGC | | 37 | CTG |
| 48 | E51SaCas9KKH | GAGTAACAGTCTGAGTAGG | 64 | E51SaCas9KKH | GATGTTGGAGGTACCTGCT |
| | 22 | AGC | | 38 | CTG |
| 48 | E51SaCas9KKH | GAGTAACAGTCTGAGTAGG | 65 | E51SaCas9KKH | AAATGCCATCTTCCTTGAT |
| | 22 | AGC | | 39 | GTT |
| 48 | E51SaCas9KKH | GAGTAACAGTCTGAGTAGG | 66 | E51SaCas9KKH | CCAAACTAGAAATGCCATC |
| | 22 | AGC | | 40 | TTC |
| 48 | E51SaCas9KKH | GAGTAACAGTCTGAGTAGG | 67 | E51SaCas9KKH | AGGAAACTGCCATCTCCAA |
| | 22 | AGC | | 41 | ACT |
| 48 | E51SaCas9KKH | GAGTAACAGTCTGAGTAGG | 68 | E51SaCas9KKH | CTGTTACTCTGGTGACACA |
| | 22 | AGC | | 42 | ACC |
| 48 | E51SaCas9KKH | GAGTAACAGTCTGAGTAGG | 69 | E51SaCas9KKH | TAGCTCCTACTCAGACTGT |
| | 22 | AGC | | 43 | TAC |
| 53 | E51SaCas9KKH | TCAACGAGATGATCATCAA | 54 | E51SaCas9KKH | GTGACCTTGAGGATATCAA |
| | 27 | GCA | | 28 | CGA |
| 53 | E51SaCas9KKH | TCAACGAGATGATCATCAA | 55 | E51SaCas9KKH | TGGGTGACCTTGAGGATAT |
| | 27 | GCA | | 29 | CAA |
| 53 | E51SaCas9KKH | TCAACGAGATGATCATCAA | 56 | E51SaCas9KKH | GAGGGTGATGGTGGGTGAC |
| | 27 | GCA | | 30 | CTT |
| 53 | E51SaCas9KKH | TCAACGAGATGATCATCAA | 57 | E51SaCas9KKH | TATAAAATCACAGAGGGTG |
| | 27 | GCA | | 31 | ATG |
| 53 | E51SaCas9KKH | TCAACGAGATGATCATCAA | 58 | E51SaCas9KKH | AAGTTATAAAATCACAGAG |
| | 27 | GCA | | 32 | GGT |
| 53 | E51SaCas9KKH | TCAACGAGATGATCATCAA | 59 | E51SaCas9KKH | ATCAAGTTATAAAATCACA |
| | 27 | GCA | | 33 | GAG |
| 53 | E51SaCas9KKH | TCAACGAGATGATCATCAA | 60 | E51SaCas9KKH | TTGATCAAGTTATAAAATC |
| | 27 | GCA | | 34 | ACA |
| 53 | E51SaCas9KKH | TCAACGAGATGATCATCAA | 61 | E51SaCas9KKH | CTTTCTCTGCTTGATCAAG |
| | 27 | GCA | | 35 | TTA |
| 53 | E51SaCas9KKH | TCAACGAGATGATCATCAA | 62 | E51SaCas9KKH | CCGACTGGCTTTCTCTGCT |
| | 27 | GCA | | 36 | TGA |
| 53 | E51SaCas9KKH | TCAACGAGATGATCATCAA | 63 | E51SaCas9KKH | ACTTACCGACTGGCTTTCT |
| | 27 | GCA | | 37 | CTG |
| 53 | E51SaCas9KKH | TCAACGAGATGATCATCAA | 64 | E51SaCas9KKH | GATGTTGGAGGTACCTGCT |
| | 27 | GCA | | 38 | CTG |
| 53 | E51SaCas9KKH | TCAACGAGATGATCATCAA | 65 | E51SaCas9KKH | AAATGCCATCTTCCTTGAT |
| | 27 | GCA | | 39 | GTT |
| 53 | E51SaCas9KKH | TCAACGAGATGATCATCAA | 66 | E51SaCas9KKH | CCAAACTAGAAATGCCATC |
| | 27 | GCA | | 40 | TTC |
| 53 | E51SaCas9KKH | TCAACGAGATGATCATCAA | 67 | E51SaCas9KKH | AGGAAACTGCCATCTCCAA |
| | 27 | GCA | | 41 | ACT |
| 53 | E51SaCas9KKH | TCAACGAGATGATCATCAA | 68 | E51SaCas9KKH | CTGTTACTCTGGTGACACA |
| | 27 | GCA | | 42 | ACC |
| 53 | E51SaCas9KKH | TCAACGAGATGATCATCAA | 69 | E51SaCas9KKH | TAGCTCCTACTCAGACTGT |
| | 27 | GCA | | 43 | TAC |
| 54 | E51SaCas9KKH | GTGACCTTGAGGATATCAA | 55 | E51SaCas9KKH | TGGGTGACCTTGAGGATAT |
| | 28 | CGA | | 29 | CAA |
| 54 | E51SaCas9KKH | GTGACCTTGAGGATATCAA | 56 | E51SaCas9KKH | GAGGGTGATGGTGGGTGAC |
| | 28 | CGA | | 30 | CTT |
| 54 | E51SaCas9KKH | GTGACCTTGAGGATATCAA | 57 | E51SaCas9KKH | TATAAAATCACAGAGGGTG |
| | 28 | CGA | | 31 | ATG |
| 54 | E51SaCas9KKH | GTGACCTTGAGGATATCAA | 58 | E51SaCas9KKH | AAGTTATAAAATCACAGAG |
| | 28 | CGA | | 32 | GGT |
| 54 | E51SaCas9KKH | GTGACCTTGAGGATATCAA | 59 | E51SaCas9KKH | ATCAAGTTATAAAATCACA |
| | 28 | CGA | | 33 | GAG |
| 54 | E51SaCas9KKH | GTGACCTTGAGGATATCAA | 60 | E51SaCas9KKH | TTGATCAAGTTATAAAATC |
| | 28 | CGA | | 34 | ACA |
| 54 | E51SaCas9KKH | GTGACCTTGAGGATATCAA | 61 | E51SaCas9KKH | CTTTCTCTGCTTGATCAAG |
| | 28 | CGA | | 35 | TTA |
| 54 | E51SaCas9KKH | GTGACCTTGAGGATATCAA | 62 | E51SaCas9KKH | CCGACTGGCTTTCTCTGCT |
| | 28 | CGA | | 36 | TGA |
| 54 | E51SaCas9KKH | GTGACCTTGAGGATATCAA | 63 | E51SaCas9KKH | ACTTACCGACTGGCTTTCT |
| | 28 | CGA | | 37 | CTG |
| 54 | E51SaCas9KKH | GTGACCTTGAGGATATCAA | 64 | E51SaCas9KKH | GATGTTGGAGGTACCTGCT |
| | 28 | CGA | | 38 | CTG |
| 54 | E51SaCas9KKH | GTGACCTTGAGGATATCAA | 65 | E51SaCas9KKH | AAATGCCATCTTCCTTGAT |
| | 28 | CGA | | 39 | GTT |
| 54 | E51SaCas9KKH | GTGACCTTGAGGATATCAA | 66 | E51SaCas9KKH | CCAAACTAGAAATGCCATC |
| | 28 | CGA | | 40 | TTC |
| 54 | E51SaCas9KKH | GTGACCTTGAGGATATCAA | 67 | E51SaCas9KKH | AGGAAACTGCCATCTCCAA |
| | 28 | CGA | | 41 | ACT |
| 54 | E51SaCas9KKH | GTGACCTTGAGGATATCAA | 68 | E51SaCas9KKH | CTGTTACTCTGGTGACACA |
| | 28 | CGA | | 42 | ACC |
| 54 | E51SaCas9KKH | GTGACCTTGAGGATATCAA | 69 | E51SaCas9KKH | TAGCTCCTACTCAGACTGT |
| | 28 | CGA | | 43 | TAC |
| 55 | E51SaCas9KKH | TGGGTGACCTTGAGGATAT | 56 | E51SaCas9KKH | GAGGGTGATGGTGGGTGAC |
| | 29 | CAA | | 30 | CTT |
| 55 | E51SaCas9KKH | TGGGTGACCTTGAGGATAT | 57 | E51SaCas9KKH | TATAAAATCACAGAGGGTG |
| | 29 | CAA | | 31 | ATG |
| 55 | E51SaCas9KKH | TGGGTGACCTTGAGGATAT | 58 | E51SaCas9KKH | AAGTTATAAAATCACAGAG |
| | 29 | CAA | | 32 | GGT |
| 55 | E51SaCas9KKH | TGGGTGACCTTGAGGATAT | 59 | E51SaCas9KKH | ATCAAGTTATAAAATCACA |
| | 29 | CAA | | 33 | GAG |
| 55 | E51SaCas9KKH | TGGGTGACCTTGAGGATAT | 60 | E51SaCas9KKH | TTGATCAAGTTATAAAATC |
| | 29 | CAA | | 34 | ACA |
| 55 | E51SaCas9KKH | TGGGTGACCTTGAGGATAT | 61 | E51SaCas9KKH | CTTTCTCTGCTTGATCAAG |
| | 29 | CAA | | 35 | TTA |
| 55 | E51SaCas9KKH | TGGGTGACCTTGAGGATAT | 62 | E51SaCas9KKH | CCGACTGGCTTTCTCTGCT |
| | 29 | CAA | | 36 | TGA |
| 55 | E51SaCas9KKH | TGGGTGACCTTGAGGATAT | 63 | E51SaCas9KKH | ACTTACCGACTGGCTTTCT |
| | 29 | CAA | | 37 | CTG |
| 55 | E51SaCas9KKH | TGGGTGACCTTGAGGATAT | 64 | E51SaCas9KKH | GATGTTGGAGGTACCTGCT |
| | 29 | CAA | | 38 | CTG |
| 55 | E51SaCas9KKH | TGGGTGACCTTGAGGATAT | 65 | E51SaCas9KKH | AAATGCCATCTTCCTTGAT |
| | 29 | CAA | | 39 | GTT |
| 55 | E51SaCas9KKH | TGGGTGACCTTGAGGATAT | 66 | E51SaCas9KKH | CCAAACTAGAAATGCCATC |
| | 29 | CAA | | 40 | TTC |
| 55 | E51SaCas9KKH | TGGGTGACCTTGAGGATAT | 67 | E51SaCas9KKH | AGGAAACTGCCATCTCCAA |
| | 29 | CAA | | 41 | ACT |
| 55 | E51SaCas9KKH | TGGGTGACCTTGAGGATAT | 68 | E51SaCas9KKH | CTGTTACTCTGGTGACACA |
| | 29 | CAA | | 42 | ACC |
| 55 | E51SaCas9KKH | TGGGTGACCTTGAGGATAT | 69 | E51SaCas9KKH | TAGCTCCTACTCAGACTGT |
| | 29 | CAA | | 43 | TAC |
| 56 | E51SaCas9KKH | GAGGGTGATGGTGGGTGAC | 57 | E51SaCas9KKH | TATAAAATCACAGAGGGTG |
| | 30 | CTT | | 31 | ATG |
| 56 | E51SaCas9KKH | GAGGGTGATGGTGGGTGAC | 58 | E51SaCas9KKH | AAGTTATAAAATCACAGAG |
| | 30 | CTT | | 32 | GGT |
| 56 | E51SaCas9KKH | GAGGGTGATGGTGGGTGAC | 59 | E51SaCas9KKH | ATCAAGTTATAAAATCACA |
| | 30 | CTT | | 33 | GAG |
| 56 | E51SaCas9KKH | GAGGGTGATGGTGGGTGAC | 60 | E51SaCas9KKH | TTGATCAAGTTATAAAATC |
| | 30 | CTT | | 34 | ACA |
| 56 | E51SaCas9KKH | GAGGGTGATGGTGGGTGAC | 61 | E51SaCas9KKH | CTTTCTCTGCTTGATCAAG |
| | 30 | CTT | | 35 | TTA |
| 56 | E51SaCas9KKH | GAGGGTGATGGTGGGTGAC | 62 | E51SaCas9KKH | CCGACTGGCTTTCTCTGCT |
| | 30 | CTT | | 36 | TGA |
| 56 | E51SaCas9KKH | GAGGGTGATGGTGGGTGAC | 63 | E51SaCas9KKH | ACTTACCGACTGGCTTTCT |
| | 30 | CTT | | 37 | CTG |
| 56 | E51SaCas9KKH | GAGGGTGATGGTGGGTGAC | 64 | E51SaCas9KKH | GATGTTGGAGGTACCTGCT |
| | 30 | CTT | | 38 | CTG |
| 56 | E51SaCas9KKH | GAGGGTGATGGTGGGTGAC | 65 | E51SaCas9KKH | AAATGCCATCTTCCTTGAT |
| | 30 | CTT | | 39 | GTT |
| 56 | E51SaCas9KKH | GAGGGTGATGGTGGGTGAC | 66 | E51SaCas9KKH | CCAAACTAGAAATGCCATC |
| | 30 | CTT | | 40 | TTC |
| 56 | E51SaCas9KKH | GAGGGTGATGGTGGGTGAC | 67 | E51SaCas9KKH | AGGAAACTGCCATCTCCAA |
| | 30 | CTT | | 41 | ACT |
| 56 | E51SaCas9KKH | GAGGGTGATGGTGGGTGAC | 68 | E51SaCas9KKH | CTGTTACTCTGGTGACACA |
| | 30 | CTT | | 42 | ACC |
| 56 | E51SaCas9KKH | GAGGGTGATGGTGGGTGAC | 69 | E51SaCas9KKH | TAGCTCCTACTCAGACTGT |
| | 30 | CTT | | 43 | TAC |
| 57 | E51SaCas9KKH | TATAAAATCACAGAGGGTG | 58 | E51SaCas9KKH | AAGTTATAAAATCACAGAG |
| | 31 | ATG | | 32 | GGT |
| 57 | E51SaCas9KKH | TATAAAATCACAGAGGGTG | 59 | E51SaCas9KKH | ATCAAGTTATAAAATCACA |
| | 31 | ATG | | 33 | GAG |
| 57 | E51SaCas9KKH | TATAAAATCACAGAGGGTG | 60 | E51SaCas9KKH | TTGATCAAGTTATAAAATC |
| | 31 | ATG | | 34 | ACA |
| 57 | E51SaCas9KKH | TATAAAATCACAGAGGGTG | 61 | E51SaCas9KKH | CTTTCTCTGCTTGATCAAG |
| | 31 | ATG | | 35 | TTA |
| 57 | E51SaCas9KKH | TATAAAATCACAGAGGGTG | 62 | E51SaCas9KKH | CCGACTGGCTTTCTCTGCT |
| | 31 | ATG | | 36 | TGA |
| 57 | E51SaCas9KKH | TATAAAATCACAGAGGGTG | 63 | E51SaCas9KKH | ACTTACCGACTGGCTTTCT |
| | 31 | ATG | | 37 | CTG |
| 57 | E51SaCas9KKH | TATAAAATCACAGAGGGTG | 64 | E51SaCas9KKH | GATGTTGGAGGTACCTGCT |
| | 31 | ATG | | 38 | CTG |
| 57 | E51SaCas9KKH | TATAAAATCACAGAGGGTG | 65 | E51SaCas9KKH | AAATGCCATCTTCCTTGAT |
| | 31 | ATG | | 39 | GTT |
| 57 | E51SaCas9KKH | TATAAAATCACAGAGGGTG | 66 | E51SaCas9KKH | CCAAACTAGAAATGCCATC |
| | 31 | ATG | | 40 | TTC |
| 57 | E51SaCas9KKH | TATAAAATCACAGAGGGTG | 67 | E51SaCas9KKH | AGGAAACTGCCATCTCCAA |
| | 31 | ATG | | 41 | ACT |
| 57 | E51SaCas9KKH | TATAAAATCACAGAGGGTG | 68 | E51SaCas9KKH | CTGTTACTCTGGTGACACA |
| | 31 | ATG | | 42 | ACC |
| 57 | E51SaCas9KKH | TATAAAATCACAGAGGGTG | 69 | E51SaCas9KKH | TAGCTCCTACTCAGACTGT |
| | 31 | ATG | | 43 | TAC |
| 58 | E51SaCas9KKH | AAGTTATAAAATCACAGAG | 59 | E51SaCas9KKH | ATCAAGTTATAAAATCACA |
| | 32 | GGT | | 33 | GAG |
| 58 | E51SaCas9KKH | AAGTTATAAAATCACAGAG | 60 | E51SaCas9KKH | TTGATCAAGTTATAAAATC |
| | 32 | GGT | | 34 | ACA |
| 58 | E51SaCas9KKH | AAGTTATAAAATCACAGAG | 61 | E51SaCas9KKH | CTTTCTCTGCTTGATCAAG |
| | 32 | GGT | | 35 | TTA |
| 58 | E51SaCas9KKH | AAGTTATAAAATCACAGAG | 62 | E51SaCas9KKH | CCGACTGGCTTTCTCTGCT |
| | 32 | GGT | | 36 | TGA |
| 58 | E51SaCas9KKH | AAGTTATAAAATCACAGAG | 63 | E51SaCas9KKH | ACTTACCGACTGGCTTTCT |
| | 32 | GGT | | 37 | CTG |
| 58 | E51SaCas9KKH | AAGTTATAAAATCACAGAG | 64 | E51SaCas9KKH | GATGTTGGAGGTACCTGCT |
| | 32 | GGT | | 38 | CTG |
| 58 | E51SaCas9KKH | AAGTTATAAAATCACAGAG | 65 | E51SaCas9KKH | AAATGCCATCTTCCTTGAT |
| | 32 | GGT | | 39 | GTT |
| 58 | E51SaCas9KKH | AAGTTATAAAATCACAGAG | 66 | E51SaCas9KKH | CCAAACTAGAAATGCCATC |
| | 32 | GGT | | 40 | TTC |
| 58 | E51SaCas9KKH | AAGTTATAAAATCACAGAG | 67 | E51SaCas9KKH | AGGAAACTGCCATCTCCAA |
| | 32 | GGT | | 41 | ACT |
| 58 | E51SaCas9KKH | AAGTTATAAAATCACAGAG | 68 | E51SaCas9KKH | CTGTTACTCTGGTGACACA |
| | 32 | GGT | | 42 | ACC |
| 58 | E51SaCas9KKH | AAGTTATAAAATCACAGAG | 69 | E51SaCas9KKH | TAGCTCCTACTCAGACTGT |
| | 32 | GGT | | 43 | TAC |
| 59 | E51SaCas9KKH | ATCAAGTTATAAAATCACA | 60 | E51SaCas9KKH | TTGATCAAGTTATAAAATC |
| | 33 | GAG | | 34 | ACA |
| 59 | E51SaCas9KKH | ATCAAGTTATAAAATCACA | 61 | E51SaCas9KKH | CTTTCTCTGCTTGATCAAG |
| | 33 | GAG | | 35 | TTA |
| 59 | E51SaCas9KKH | ATCAAGTTATAAAATCACA | 62 | E51SaCas9KKH | CCGACTGGCTTTCTCTGCT |
| | 33 | GAG | | 36 | TGA |
| 59 | E51SaCas9KKH | ATCAAGTTATAAAATCACA | 63 | E51SaCas9KKH | ACTTACCGACTGGCTTTCT |
| | 33 | GAG | | 37 | CTG |
| 59 | E51SaCas9KKH | ATCAAGTTATAAAATCACA | 64 | E51SaCas9KKH | GATGTTGGAGGTACCTGCT |
| | 33 | GAG | | 38 | CTG |
| 59 | E51SaCas9KKH | ATCAAGTTATAAAATCACA | 65 | E51SaCas9KKH | AAATGCCATCTTCCTTGAT |
| | 33 | GAG | | 39 | GTT |
| 59 | E51SaCas9KKH | ATCAAGTTATAAAATCACA | 66 | E51SaCas9KKH | CCAAACTAGAAATGCCATC |
| | 33 | GAG | | 40 | TTC |
| 59 | E51SaCas9KKH | ATCAAGTTATAAAATCACA | 67 | E51SaCas9KKH | AGGAAACTGCCATCTCCAA |
| | 33 | GAG | | 41 | ACT |
| 59 | E51SaCas9KKH | ATCAAGTTATAAAATCACA | 68 | E51SaCas9KKH | CTGTTACTCTGGTGACACA |
| | 33 | GAG | | 42 | ACC |
| 59 | E51SaCas9KKH | ATCAAGTTATAAAATCACA | 69 | E51SaCas9KKH | TAGCTCCTACTCAGACTGT |
| | 33 | GAG | | 43 | TAC |
| 60 | E51SaCas9KKH | TTGATCAAGTTATAAAATC | 61 | E51SaCas9KKH | CTTTCTCTGCTTGATCAAG |
| | 34 | ACA | | 35 | TTA |
| 60 | E51SaCas9KKH | TTGATCAAGTTATAAAATC | 62 | E51SaCas9KKH | CCGACTGGCTTTCTCTGCT |
| | 34 | ACA | | 36 | TGA |
| 60 | E51SaCas9KKH | TTGATCAAGTTATAAAATC | 63 | E51SaCas9KKH | ACTTACCGACTGGCTTTCT |
| | 34 | ACA | | 37 | CTG |
| 60 | E51SaCas9KKH | TTGATCAAGTTATAAAATC | 64 | E51SaCas9KKH | GATGTTGGAGGTACCTGCT |
| | 34 | ACA | | 38 | CTG |
| 60 | E51SaCas9KKH | TTGATCAAGTTATAAAATC | 65 | E51SaCas9KKH | AAATGCCATCTTCCTTGAT |
| | 34 | ACA | | 39 | GTT |
| 60 | E51SaCas9KKH | TTGATCAAGTTATAAAATC | 66 | E51SaCas9KKH | CCAAACTAGAAATGCCATC |
| | 34 | ACA | | 40 | TTC |
| 60 | E51SaCas9KKH | TTGATCAAGTTATAAAATC | 67 | E51SaCas9KKH | AGGAAACTGCCATCTCCAA |
| | 34 | ACA | | 41 | ACT |
| 60 | E51SaCas9KKH | TTGATCAAGTTATAAAATC | 68 | E51SaCas9KKH | CTGTTACTCTGGTGACACA |
| | 34 | ACA | | 42 | ACC |
| 60 | E51SaCas9KKH | TTGATCAAGTTATAAAATC | 69 | E51SaCas9KKH | TAGCTCCTACTCAGACTGT |
| | 34 | ACA | | 43 | TAC |
| 61 | E51SaCas9KKH | CTTTCTCTGCTTGATCAAG | 62 | E51SaCas9KKH | CCGACTGGCTTTCTCTGCT |
| | 35 | TTA | | 36 | TGA |
| 61 | E51SaCas9KKH | CTTTCTCTGCTTGATCAAG | 63 | E51SaCas9KKH | ACTTACCGACTGGCTTTCT |
| | 35 | TTA | | 37 | CTG |
| 61 | E51SaCas9KKH | CTTTCTCTGCTTGATCAAG | 64 | E51SaCas9KKH | GATGTTGGAGGTACCTGCT |
| | 35 | TTA | | 38 | CTG |
| 61 | E51SaCas9KKH | CTTTCTCTGCTTGATCAAG | 65 | E51SaCas9KKH | AAATGCCATCTTCCTTGAT |
| | 35 | TTA | | 39 | GTT |
| 61 | E51SaCas9KKH | CTTTCTCTGCTTGATCAAG | 66 | E51SaCas9KKH | CCAAACTAGAAATGCCATC |
| | 35 | TTA | | 40 | TTC |
| 61 | E51SaCas9KKH | CTTTCTCTGCTTGATCAAG | 67 | E51SaCas9KKH | AGGAAACTGCCATCTCCAA |
| | 35 | TTA | | 41 | ACT |
| 61 | E51SaCas9KKH | CTTTCTCTGCTTGATCAAG | 68 | E51SaCas9KKH | CTGTTACTCTGGTGACACA |
| | 35 | TTA | | 42 | ACC |
| 61 | E51SaCas9KKH | CTTTCTCTGCTTGATCAAG | 69 | E51SaCas9KKH | TAGCTCCTACTCAGACTGT |
| | 35 | TTA | | 43 | TAC |
| 62 | E51SaCas9KKH | CCGACTGGCTTTCTCTGCT | 63 | E51SaCas9KKH | ACTTACCGACTGGCTTTCT |
| | 36 | TGA | | 37 | CTG |
| 62 | E51SaCas9KKH | CCGACTGGCTTTCTCTGCT | 64 | E51SaCas9KKH | GATGTTGGAGGTACCTGCT |
| | 36 | TGA | | 38 | CTG |
| 62 | E51SaCas9KKH | CCGACTGGCTTTCTCTGCT | 65 | E51SaCas9KKH | AAATGCCATCTTCCTTGAT |
| | 36 | TGA | | 39 | GTT |
| 62 | E51SaCas9KKH | CCGACTGGCTTTCTCTGCT | 66 | E51SaCas9KKH | CCAAACTAGAAATGCCATC |
| | 36 | TGA | | 40 | TTC |
| 62 | E51SaCas9KKH | CCGACTGGCTTTCTCTGCT | 67 | E51SaCas9KKH | AGGAAACTGCCATCTCCAA |
| | 36 | TGA | | 41 | ACT |
| 62 | E51SaCas9KKH | CCGACTGGCTTTCTCTGCT | 68 | E51SaCas9KKH | CTGTTACTCTGGTGACACA |
| | 36 | TGA | | 42 | ACC |
| 62 | E51SaCas9KKH | CCGACTGGCTTTCTCTGCT | 69 | E51SaCas9KKH | TAGCTCCTACTCAGACTGT |
| | 36 | TGA | | 43 | TAC |
| 63 | E51SaCas9KKH | ACTTACCGACTGGCTTTCT | 64 | E51SaCas9KKH | GATGTTGGAGGTACCTGCT |
| | 37 | CTG | | 38 | CTG |
| 63 | E51SaCas9KKH | ACTTACCGACTGGCTTTCT | 65 | E51SaCas9KKH | AAATGCCATCTTCCTTGAT |
| | 37 | CTG | | 39 | GTT |
| 63 | E51SaCas9KKH | ACTTACCGACTGGCTTTCT | 66 | E51SaCas9KKH | CCAAACTAGAAATGCCATC |
| | 37 | CTG | | 40 | TTC |
| 63 | E51SaCas9KKH | ACTTACCGACTGGCTTTCT | 67 | E51SaCas9KKH | AGGAAACTGCCATCTCCAA |
| | 37 | CTG | | 41 | ACT |
| 63 | E51SaCas9KKH | ACTTACCGACTGGCTTTCT | 68 | E51SaCas9KKH | CTGTTACTCTGGTGACACA |
| | 37 | CTG | | 42 | ACC |
| 63 | E51SaCas9KKH | ACTTACCGACTGGCTTTCT | 69 | E51SaCas9KKH | TAGCTCCTACTCAGACTGT |
| | 37 | CTG | | 43 | TAC |
| 64 | E51SaCas9KKH | GATGTTGGAGGTACCTGCT | 65 | E51SaCas9KKH | AAATGCCATCTTCCTTGAT |
| | 38 | CTG | | 39 | GTT |
| 64 | E51SaCas9KKH | GATGTTGGAGGTACCTGCT | 66 | E51SaCas9KKH | CCAAACTAGAAATGCCATC |
| | 38 | CTG | | 40 | TTC |
| 64 | E51SaCas9KKH | GATGTTGGAGGTACCTGCT | 67 | E51SaCas9KKH | AGGAAACTGCCATCTCCAA |
| | 38 | CTG | | 41 | ACT |
| 64 | E51SaCas9KKH | GATGTTGGAGGTACCTGCT | 68 | E51SaCas9KKH | CTGTTACTCTGGTGACACA |
| | 38 | CTG | | 42 | ACC |
| 64 | E51SaCas9KKH | GATGTTGGAGGTACCTGCT | 69 | E51SaCas9KKH | TAGCTCCTACTCAGACTGT |
| | 38 | CTG | | 43 | TAC |
| 65 | E51SaCas9KKH | AAATGCCATCTTCCTTGAT | 66 | E51SaCas9KKH | CCAAACTAGAAATGCCATC |
| | 39 | GTT | | 40 | TTC |
| 65 | E51SaCas9KKH | AAATGCCATCTTCCTTGAT | 67 | E51SaCas9KKH | AGGAAACTGCCATCTCCAA |
| | 39 | GTT | | 41 | ACT |
| 65 | E51SaCas9KKH | AAATGCCATCTTCCTTGAT | 68 | E51SaCas9KKH | CTGTTACTCTGGTGACACA |
| | 39 | GTT | | 42 | ACC |
| 65 | E51SaCas9KKH | AAATGCCATCTTCCTTGAT | 69 | E51SaCas9KKH | TAGCTCCTACTCAGACTGT |
| | 39 | GTT | | 43 | TAC |
| 66 | E51SaCas9KKH | CCAAACTAGAAATGCCATC | 67 | E51SaCas9KKH | AGGAAACTGCCATCTCCAA |
| | 40 | TTC | | 41 | ACT |
| 66 | E51SaCas9KKH | CCAAACTAGAAATGCCATC | 68 | E51SaCas9KKH | CTGTTACTCTGGTGACACA |
| | 40 | TTC | | 42 | ACC |
| 66 | E51SaCas9KKH | CCAAACTAGAAATGCCATC | 69 | E51SaCas9KKH | TAGCTCCTACTCAGACTGT |
| | 40 | TTC | | 43 | TAC |
| 67 | E51SaCas9KKH | AGGAAACTGCCATCTCCAA | 68 | E51SaCas9KKH | CTGTTACTCTGGTGACACA |
| | 41 | ACT | | 42 | ACC |
| 67 | E51SaCas9KKH | AGGAAACTGCCATCTCCAA | 69 | E51SaCas9KKH | TAGCTCCTACTCAGACTGT |
| | 41 | ACT | | 43 | TAC |
| 68 | E51SaCas9KKH | CTGTTACTCTGGTGACACA | 69 | E51SaCas9KKH | TAGCTCCTACTCAGACTGT |
| | 42 | ACC | | 43 | TAC |

TABLE 4C
(3). Exon 51 SluCas9 guides:

| Seq | Guide ID or | |
|---|---|---|
| ID No. | Guide RNA Name | Guide Sequence |
| 243 | E51Slu1 | TGATCATCTCGTTGATATCCTC |
| 244 | E51Slu2 | TTGATCAAGCAGAGAAAGCCAG |
| 245 | E51Slu3 | AGTCGGTAAGTTCTGTCCAAGC |
| 246 | E51Slu4 | GCCCGGTTGAAATCTGCCAGAG |
| 247 | E51Slu5 | CAGAGCAGGTACCTCCAACATC |
| 248 | E51Slu6 | GGTACCTCCAACATCAAGGAAG |
| 249 | E51Slu7 | CAAGGAAGATGGCATTTCTAGT |
| 250 | E51Slu8 | AGATGGCATTTCTAGTTTGGAG |
| 251 | E51Slu9 | ATGGCAGTTTCCTTAGTAACCA |
| 252 | E51Slu10 | GTCACCAGAGTAACAGTCTGAG |
| 253 | E51Slu15 | CAACGAGATGATCATCAAGCAG |
| 254 | E51Slu16 | GAGGGTGATGGTGGGTGACCTT |
| 255 | E51Slu17 | ATAAAATCACAGAGGGTGATGG |
| 256 | E51Slu18 | TATAAAATCACAGAGGGTGATG |
| 257 | E51Slu19 | AGTTATAAAATCACAGAGGGTG |
| 258 | E51Slu20 | TGATCAAGTTATAAAATCACAG |
| 259 | E51Slu21 | TTGATCAAGTTATAAAATCACA |
| 260 | E51Slu22 | GGGCTTGGACAGAACTTACCGA |
| 261 | E51Slu23 | CTCTGGCAGATTTCAACCGGGC |
| 262 | E51Slu24 | ACCTGCTCTGGCAGATTTCAAC |
| 263 | E51Slu25 | TACCTGCTCTGGCAGATTTCAA |
| 264 | E51Slu26 | CTTGATGTTGGAGGTACCTGCT |
| 265 | E51Slu27 | AATGCCATCTTCCTTGATGTTG |
| 266 | E51Slu28 | AGAAATGCCATCTTCCTTGATG |
| 267 | E51Slu29 | GGTGACACAACCTGTGGTTACT |
| 268 | E51Slu30 | TGTTACTCTGGTGACACAACCT |
| 269 | E51Slu31 | AGCTCCTACTCAGACTGTTACT |

TABLE 4D
(4). Exon 51 SluCas9 pairs:

| Guide 1 | | | Guide 2 | | |
|---|---|---|---|---|---|
| Seq ID | | | Seq ID | | |
| No. | Guide 1 | Guide 1 Sequence | No. | Guide 2 | Guide 2 Sequence |
| 243 | E51SL1 | TGATCATCTCGTTGATATCCTC | 244 | E51SL2 | TTGATCAAGCAGAGAAAGCCAG |
| 243 | E51SL1 | TGATCATCTCGTTGATATCCTC | 245 | E51SL3 | AGTCGGTAAGTTCTGTCCAAGC |
| 243 | E51SL1 | TGATCATCTCGTTGATATCCTC | 246 | E51SL4 | GCCCGGTTGAAATCTGCCAGAG |
| 243 | E51SL1 | TGATCATCTCGTTGATATCCTC | 247 | E51SL5 | CAGAGCAGGTACCTCCAACATC |
| 243 | E51SL1 | TGATCATCTCGTTGATATCCTC | 248 | E51SL6 | GGTACCTCCAACATCAAGGAAG |
| 243 | E51SL1 | TGATCATCTCGTTGATATCCTC | 249 | E51SL7 | CAAGGAAGATGGCATTTCTAGT |
| 243 | E51SL1 | TGATCATCTCGTTGATATCCTC | 250 | E51SL8 | AGATGGCATTTCTAGTTTGGAG |
| 243 | E51SL1 | TGATCATCTCGTTGATATCCTC | 251 | E51SL9 | ATGGCAGTTTCCTTAGTAACCA |
| 243 | E51SL1 | TGATCATCTCGTTGATATCCTC | 252 | E51SL10 | GTCACCAGAGTAACAGTCTGAG |
| 243 | E51SL1 | TGATCATCTCGTTGATATCCTC | 253 | E51SL15 | CAACGAGATGATCATCAAGCAG |
| 243 | E51SL1 | TGATCATCTCGTTGATATCCTC | 254 | E51SL16 | GAGGGTGATGGTGGGTGACCTT |
| 243 | E51SL1 | TGATCATCTCGTTGATATCCTC | 255 | E51SL17 | ATAAAATCACAGAGGGTGATGG |
| 243 | E51SL1 | TGATCATCTCGTTGATATCCTC | 256 | E51SL18 | TATAAAATCACAGAGGGTGATG |
| 243 | E51SL1 | TGATCATCTCGTTGATATCCTC | 257 | E51SL19 | AGTTATAAAATCACAGAGGGTG |
| 243 | E51SL1 | TGATCATCTCGTTGATATCCTC | 258 | E51SL20 | TGATCAAGTTATAAAATCACAG |
| 243 | E51SL1 | TGATCATCTCGTTGATATCCTC | 259 | E51SL21 | TTGATCAAGTTATAAAATCACA |
| 243 | E51SL1 | TGATCATCTCGTTGATATCCTC | 260 | E51SL22 | GGGCTTGGACAGAACTTACCGA |
| 243 | E51SL1 | TGATCATCTCGTTGATATCCTC | 261 | E51SL23 | CTCTGGCAGATTTCAACCGGGC |
| 243 | E51SL1 | TGATCATCTCGTTGATATCCTC | 262 | E51SL24 | ACCTGCTCTGGCAGATTTCAAC |
| 243 | E51SL1 | TGATCATCTCGTTGATATCCTC | 263 | E51SL25 | TACCTGCTCTGGCAGATTTCAA |
| 243 | E51SL1 | TGATCATCTCGTTGATATCCTC | 264 | E51SL26 | CTTGATGTTGGAGGTACCTGCT |
| 243 | E51SL1 | TGATCATCTCGTTGATATCCTC | 265 | E51SL27 | AATGCCATCTTCCTTGATGTTG |
| 243 | E51SL1 | TGATCATCTCGTTGATATCCTC | 266 | E51SL28 | AGAAATGCCATCTTCCTTGATG |
| 243 | E51SL1 | TGATCATCTCGTTGATATCCTC | 267 | E51SL29 | GGTGACACAACCTGTGGTTACT |
| 243 | E51SL1 | TGATCATCTCGTTGATATCCTC | 268 | E51SL30 | TGTTACTCTGGTGACACAACCT |
| 243 | E51SL1 | TGATCATCTCGTTGATATCCTC | 269 | E51SL31 | AGCTCCTACTCAGACTGTTACT |
| 244 | E51SL2 | TTGATCAAGCAGAGAAAGCCAG | 245 | E51SL3 | AGTCGGTAAGTTCTGTCCAAGC |
| 244 | E51SL2 | TTGATCAAGCAGAGAAAGCCAG | 246 | E51SL4 | GCCCGGTTGAAATCTGCCAGAG |
| 244 | E51SL2 | TTGATCAAGCAGAGAAAGCCAG | 247 | E51SL5 | CAGAGCAGGTACCTCCAACATC |
| 244 | E51SL2 | TTGATCAAGCAGAGAAAGCCAG | 248 | E51SL6 | GGTACCTCCAACATCAAGGAAG |
| 244 | E51SL2 | TTGATCAAGCAGAGAAAGCCAG | 249 | E51SL7 | CAAGGAAGATGGCATTTCTAGT |
| 244 | E51SL2 | TTGATCAAGCAGAGAAAGCCAG | 250 | E51SL8 | AGATGGCATTTCTAGTTTGGAG |
| 244 | E51SL2 | TTGATCAAGCAGAGAAAGCCAG | 251 | E51SL9 | ATGGCAGTTTCCTTAGTAACCA |
| 244 | E51SL2 | TTGATCAAGCAGAGAAAGCCAG | 252 | E51SL10 | GTCACCAGAGTAACAGTCTGAG |
| 244 | E51SL2 | TTGATCAAGCAGAGAAAGCCAG | 253 | E51SL15 | CAACGAGATGATCATCAAGCAG |
| 244 | E51SL2 | TTGATCAAGCAGAGAAAGCCAG | 254 | E51SL16 | GAGGGTGATGGTGGGTGACCTT |
| 244 | E51SL2 | TTGATCAAGCAGAGAAAGCCAG | 255 | E51SL17 | ATAAAATCACAGAGGGTGATGG |
| 244 | E51SL2 | TTGATCAAGCAGAGAAAGCCAG | 256 | E51SL18 | TATAAAATCACAGAGGGTGATG |
| 244 | E51SL2 | TTGATCAAGCAGAGAAAGCCAG | 257 | E51SL19 | AGTTATAAAATCACAGAGGGTG |
| 244 | E51SL2 | TTGATCAAGCAGAGAAAGCCAG | 258 | E51SL20 | TGATCAAGTTATAAAATCACAG |
| 244 | E51SL2 | TTGATCAAGCAGAGAAAGCCAG | 259 | E51SL21 | TTGATCAAGTTATAAAATCACA |
| 244 | E51SL2 | TTGATCAAGCAGAGAAAGCCAG | 260 | E51SL22 | GGGCTTGGACAGAACTTACCGA |
| 244 | E51SL2 | TTGATCAAGCAGAGAAAGCCAG | 261 | E51SL23 | CTCTGGCAGATTTCAACCGGGC |
| 244 | E51SL2 | TTGATCAAGCAGAGAAAGCCAG | 262 | E51SL24 | ACCTGCTCTGGCAGATTTCAAC |
| 244 | E51SL2 | TTGATCAAGCAGAGAAAGCCAG | 263 | E51SL25 | TACCTGCTCTGGCAGATTTCAA |
| 244 | E51SL2 | TTGATCAAGCAGAGAAAGCCAG | 264 | E51SL26 | CTTGATGTTGGAGGTACCTGCT |
| 244 | E51SL2 | TTGATCAAGCAGAGAAAGCCAG | 265 | E51SL27 | AATGCCATCTTCCTTGATGTTG |
| 244 | E51SL2 | TTGATCAAGCAGAGAAAGCCAG | 266 | E51SL28 | AGAAATGCCATCTTCCTTGATG |
| 244 | E51SL2 | TTGATCAAGCAGAGAAAGCCAG | 267 | E51SL29 | GGTGACACAACCTGTGGTTACT |
| 244 | E51SL2 | TTGATCAAGCAGAGAAAGCCAG | 268 | E51SL30 | TGTTACTCTGGTGACACAACCT |
| 244 | E51SL2 | TTGATCAAGCAGAGAAAGCCAG | 269 | E51SL31 | AGCTCCTACTCAGACTGTTACT |
| 245 | E51SL3 | AGTCGGTAAGTTCTGTCCAAGC | 246 | E51SL4 | GCCCGGTTGAAATCTGCCAGAG |
| 245 | E51SL3 | AGTCGGTAAGTTCTGTCCAAGC | 247 | E51SL5 | CAGAGCAGGTACCTCCAACATC |
| 245 | E51SL3 | AGTCGGTAAGTTCTGTCCAAGC | 248 | E51SL6 | GGTACCTCCAACATCAAGGAAG |
| 245 | E51SL3 | AGTCGGTAAGTTCTGTCCAAGC | 249 | E51SL7 | CAAGGAAGATGGCATTTCTAGT |
| 245 | E51SL3 | AGTCGGTAAGTTCTGTCCAAGC | 250 | E51SL8 | AGATGGCATTTCTAGTTTGGAG |
| 245 | E51SL3 | AGTCGGTAAGTTCTGTCCAAGC | 251 | E51SL9 | ATGGCAGTTTCCTTAGTAACCA |
| 245 | E51SL3 | AGTCGGTAAGTTCTGTCCAAGC | 252 | E51SL10 | GTCACCAGAGTAACAGTCTGAG |
| 245 | E51SL3 | AGTCGGTAAGTTCTGTCCAAGC | 253 | E51SL15 | CAACGAGATGATCATCAAGCAG |
| 245 | E51SL3 | AGTCGGTAAGTTCTGTCCAAGC | 254 | E51SL16 | GAGGGTGATGGTGGGTGACCTT |
| 245 | E51SL3 | AGTCGGTAAGTTCTGTCCAAGC | 255 | E51SL17 | ATAAAATCACAGAGGGTGATGG |
| 245 | E51SL3 | AGTCGGTAAGTTCTGTCCAAGC | 256 | E51SL18 | TATAAAATCACAGAGGGTGATG |
| 245 | E51SL3 | AGTCGGTAAGTTCTGTCCAAGC | 257 | E51SL19 | AGTTATAAAATCACAGAGGGTG |
| 245 | E51SL3 | AGTCGGTAAGTTCTGTCCAAGC | 258 | E51SL20 | TGATCAAGTTATAAAATCACAG |
| 245 | E51SL3 | AGTCGGTAAGTTCTGTCCAAGC | 259 | E51SL21 | TTGATCAAGTTATAAAATCACA |
| 245 | E51SL3 | AGTCGGTAAGTTCTGTCCAAGC | 260 | E51SL22 | GGGCTTGGACAGAACTTACCGA |
| 245 | E51SL3 | AGTCGGTAAGTTCTGTCCAAGC | 261 | E51SL23 | CTCTGGCAGATTTCAACCGGGC |
| 245 | E51SL3 | AGTCGGTAAGTTCTGTCCAAGC | 262 | E51SL24 | ACCTGCTCTGGCAGATTTCAAC |
| 245 | E51SL3 | AGTCGGTAAGTTCTGTCCAAGC | 263 | E51SL25 | TACCTGCTCTGGCAGATTTCAA |
| 245 | E51SL3 | AGTCGGTAAGTTCTGTCCAAGC | 264 | E51SL26 | CTTGATGTTGGAGGTACCTGCT |
| 245 | E51SL3 | AGTCGGTAAGTTCTGTCCAAGC | 265 | E51SL27 | AATGCCATCTTCCTTGATGTTG |
| 245 | E51SL3 | AGTCGGTAAGTTCTGTCCAAGC | 266 | E51SL28 | AGAAATGCCATCTTCCTTGATG |
| 245 | E51SL3 | AGTCGGTAAGTTCTGTCCAAGC | 267 | E51SL29 | GGTGACACAACCTGTGGTTACT |
| 245 | E51SL3 | AGTCGGTAAGTTCTGTCCAAGC | 268 | E51SL30 | TGTTACTCTGGTGACACAACCT |
| 245 | E51SL3 | AGTCGGTAAGTTCTGTCCAAGC | 269 | E51SL31 | AGCTCCTACTCAGACTGTTACT |
| 246 | E51SL4 | GCCCGGTTGAAATCTGCCAGAG | 247 | E51SL5 | CAGAGCAGGTACCTCCAACATC |
| 246 | E51SL4 | GCCCGGTTGAAATCTGCCAGAG | 248 | E51SL6 | GGTACCTCCAACATCAAGGAAG |
| 246 | E51SL4 | GCCCGGTTGAAATCTGCCAGAG | 249 | E51SL7 | CAAGGAAGATGGCATTTCTAGT |
| 246 | E51SL4 | GCCCGGTTGAAATCTGCCAGAG | 250 | E51SL8 | AGATGGCATTTCTAGTTTGGAG |
| 246 | E51SL4 | GCCCGGTTGAAATCTGCCAGAG | 251 | E51SL9 | ATGGCAGTTTCCTTAGTAACCA |
| 246 | E51SL4 | GCCCGGTTGAAATCTGCCAGAG | 252 | E51SL10 | GTCACCAGAGTAACAGTCTGAG |
| 246 | E51SL4 | GCCCGGTTGAAATCTGCCAGAG | 253 | E51SL15 | CAACGAGATGATCATCAAGCAG |
| 246 | E51SL4 | GCCCGGTTGAAATCTGCCAGAG | 254 | E51SL16 | GAGGGTGATGGTGGGTGACCTT |
| 246 | E51SL4 | GCCCGGTTGAAATCTGCCAGAG | 255 | E51SL17 | ATAAAATCACAGAGGGTGATGG |
| 246 | E51SL4 | GCCCGGTTGAAATCTGCCAGAG | 256 | E51SL18 | TATAAAATCACAGAGGGTGATG |
| 246 | E51SL4 | GCCCGGTTGAAATCTGCCAGAG | 257 | E51SL19 | AGTTATAAAATCACAGAGGGTG |
| 246 | E51SL4 | GCCCGGTTGAAATCTGCCAGAG | 258 | E51SL20 | TGATCAAGTTATAAAATCACAG |
| 246 | E51SL4 | GCCCGGTTGAAATCTGCCAGAG | 259 | E51SL21 | TTGATCAAGTTATAAAATCACA |
| 246 | E51SL4 | GCCCGGTTGAAATCTGCCAGAG | 260 | E51SL22 | GGGCTTGGACAGAACTTACCGA |
| 246 | E51SL4 | GCCCGGTTGAAATCTGCCAGAG | 261 | E51SL23 | CTCTGGCAGATTTCAACCGGGC |
| 246 | E51SL4 | GCCCGGTTGAAATCTGCCAGAG | 262 | E51SL24 | ACCTGCTCTGGCAGATTTCAAC |
| 246 | E51SL4 | GCCCGGTTGAAATCTGCCAGAG | 263 | E51SL25 | TACCTGCTCTGGCAGATTTCAA |
| 246 | E51SL4 | GCCCGGTTGAAATCTGCCAGAG | 264 | E51SL26 | CTTGATGTTGGAGGTACCTGCT |
| 246 | E51SL4 | GCCCGGTTGAAATCTGCCAGAG | 265 | E51SL27 | AATGCCATCTTCCTTGATGTTG |
| 246 | E51SL4 | GCCCGGTTGAAATCTGCCAGAG | 266 | E51SL28 | AGAAATGCCATCTTCCTTGATG |
| 246 | E51SL4 | GCCCGGTTGAAATCTGCCAGAG | 267 | E51SL29 | GGTGACACAACCTGTGGTTACT |
| 246 | E51SL4 | GCCCGGTTGAAATCTGCCAGAG | 268 | E51SL30 | TGTTACTCTGGTGACACAACCT |
| 246 | E51SL4 | GCCCGGTTGAAATCTGCCAGAG | 269 | E51SL31 | AGCTCCTACTCAGACTGTTACT |
| 247 | E51SL5 | CAGAGCAGGTACCTCCAACATC | 248 | E51SL6 | GGTACCTCCAACATCAAGGAAG |
| 247 | E51SL5 | CAGAGCAGGTACCTCCAACATC | 249 | E51SL7 | CAAGGAAGATGGCATTTCTAGT |
| 247 | E51SL5 | CAGAGCAGGTACCTCCAACATC | 250 | E51SL8 | AGATGGCATTTCTAGTTTGGAG |
| 247 | E51SL5 | CAGAGCAGGTACCTCCAACATC | 251 | E51SL9 | ATGGCAGTTTCCTTAGTAACCA |
| 247 | E51SL5 | CAGAGCAGGTACCTCCAACATC | 252 | E51SL10 | GTCACCAGAGTAACAGTCTGAG |
| 247 | E51SL5 | CAGAGCAGGTACCTCCAACATC | 253 | E51SL15 | CAACGAGATGATCATCAAGCAG |
| 247 | E51SL5 | CAGAGCAGGTACCTCCAACATC | 254 | E51SL16 | GAGGGTGATGGTGGGTGACCTT |
| 247 | E51SL5 | CAGAGCAGGTACCTCCAACATC | 255 | E51SL17 | ATAAAATCACAGAGGGTGATGG |
| 247 | E51SL5 | CAGAGCAGGTACCTCCAACATC | 256 | E51SL18 | TATAAAATCACAGAGGGTGATG |
| 247 | E51SL5 | CAGAGCAGGTACCTCCAACATC | 257 | E51SL19 | AGTTATAAAATCACAGAGGGTG |
| 247 | E51SL5 | CAGAGCAGGTACCTCCAACATC | 258 | E51SL20 | TGATCAAGTTATAAAATCACAG |
| 247 | E51SL5 | CAGAGCAGGTACCTCCAACATC | 259 | E51SL21 | TTGATCAAGTTATAAAATCACA |
| 247 | E51SL5 | CAGAGCAGGTACCTCCAACATC | 260 | E51SL22 | GGGCTTGGACAGAACTTACCGA |
| 247 | E51SL5 | CAGAGCAGGTACCTCCAACATC | 261 | E51SL23 | CTCTGGCAGATTTCAACCGGGC |
| 247 | E51SL5 | CAGAGCAGGTACCTCCAACATC | 262 | E51SL24 | ACCTGCTCTGGCAGATTTCAAC |
| 247 | E51SL5 | CAGAGCAGGTACCTCCAACATC | 263 | E51SL25 | TACCTGCTCTGGCAGATTTCAA |
| 247 | E51SL5 | CAGAGCAGGTACCTCCAACATC | 264 | E51SL26 | CTTGATGTTGGAGGTACCTGCT |
| 247 | E51SL5 | CAGAGCAGGTACCTCCAACATC | 265 | E51SL27 | AATGCCATCTTCCTTGATGTTG |
| 247 | E51SL5 | CAGAGCAGGTACCTCCAACATC | 266 | E51SL28 | AGAAATGCCATCTTCCTTGATG |
| 247 | E51SL5 | CAGAGCAGGTACCTCCAACATC | 267 | E51SL29 | GGTGACACAACCTGTGGTTACT |
| 247 | E51SL5 | CAGAGCAGGTACCTCCAACATC | 268 | E51SL30 | TGTTACTCTGGTGACACAACCT |
| 247 | E51SL5 | CAGAGCAGGTACCTCCAACATC | 269 | E51SL31 | AGCTCCTACTCAGACTGTTACT |
| 248 | E51SL6 | GGTACCTCCAACATCAAGGAAG | 249 | E51SL7 | CAAGGAAGATGGCATTTCTAGT |
| 248 | E51SL6 | GGTACCTCCAACATCAAGGAAG | 250 | E51SL8 | AGATGGCATTTCTAGTTTGGAG |
| 248 | E51SL6 | GGTACCTCCAACATCAAGGAAG | 251 | E51SL9 | ATGGCAGTTTCCTTAGTAACCA |
| 248 | E51SL6 | GGTACCTCCAACATCAAGGAAG | 252 | E51SL10 | GTCACCAGAGTAACAGTCTGAG |
| 248 | E51SL6 | GGTACCTCCAACATCAAGGAAG | 253 | E51SL15 | CAACGAGATGATCATCAAGCAG |
| 248 | E51SL6 | GGTACCTCCAACATCAAGGAAG | 254 | E51SL16 | GAGGGTGATGGTGGGTGACCTT |
| 248 | E51SL6 | GGTACCTCCAACATCAAGGAAG | 255 | E51SL17 | ATAAAATCACAGAGGGTGATGG |
| 248 | E51SL6 | GGTACCTCCAACATCAAGGAAG | 256 | E51SL18 | TATAAAATCACAGAGGGTGATG |
| 248 | E51SL6 | GGTACCTCCAACATCAAGGAAG | 257 | E51SL19 | AGTTATAAAATCACAGAGGGTG |
| 248 | E51SL6 | GGTACCTCCAACATCAAGGAAG | 258 | E51SL20 | TGATCAAGTTATAAAATCACAG |
| 248 | E51SL6 | GGTACCTCCAACATCAAGGAAG | 259 | E51SL21 | TTGATCAAGTTATAAAATCACA |
| 248 | E51SL6 | GGTACCTCCAACATCAAGGAAG | 260 | E51SL22 | GGGCTTGGACAGAACTTACCGA |
| 248 | E51SL6 | GGTACCTCCAACATCAAGGAAG | 261 | E51SL23 | CTCTGGCAGATTTCAACCGGGC |
| 248 | E51SL6 | GGTACCTCCAACATCAAGGAAG | 262 | E51SL24 | ACCTGCTCTGGCAGATTTCAAC |
| 248 | E51SL6 | GGTACCTCCAACATCAAGGAAG | 263 | E51SL25 | TACCTGCTCTGGCAGATTTCAA |
| 248 | E51SL6 | GGTACCTCCAACATCAAGGAAG | 264 | E51SL26 | CTTGATGTTGGAGGTACCTGCT |
| 248 | E51SL6 | GGTACCTCCAACATCAAGGAAG | 265 | E51SL27 | AATGCCATCTTCCTTGATGTTG |
| 248 | E51SL6 | GGTACCTCCAACATCAAGGAAG | 266 | E51SL28 | AGAAATGCCATCTTCCTTGATG |
| 248 | E51SL6 | GGTACCTCCAACATCAAGGAAG | 267 | E51SL29 | GGTGACACAACCTGTGGTTACT |
| 248 | E51SL6 | GGTACCTCCAACATCAAGGAAG | 268 | E51SL30 | TGTTACTCTGGTGACACAACCT |
| 248 | E51SL6 | GGTACCTCCAACATCAAGGAAG | 269 | E51SL31 | AGCTCCTACTCAGACTGTTACT |
| 249 | E51SL7 | CAAGGAAGATGGCATTTCTAGT | 250 | E51SL8 | AGATGGCATTTCTAGTTTGGAG |
| 249 | E51SL7 | CAAGGAAGATGGCATTTCTAGT | 251 | E51SL9 | ATGGCAGTTTCCTTAGTAACCA |
| 249 | E51SL7 | CAAGGAAGATGGCATTTCTAGT | 252 | E51SL10 | GTCACCAGAGTAACAGTCTGAG |
| 249 | E51SL7 | CAAGGAAGATGGCATTTCTAGT | 253 | E51SL15 | CAACGAGATGATCATCAAGCAG |
| 249 | E51SL7 | CAAGGAAGATGGCATTTCTAGT | 254 | E51SL16 | GAGGGTGATGGTGGGTGACCTT |
| 249 | E51SL7 | CAAGGAAGATGGCATTTCTAGT | 255 | E51SL17 | ATAAAATCACAGAGGGTGATGG |
| 249 | E51SL7 | CAAGGAAGATGGCATTTCTAGT | 256 | E51SL18 | TATAAAATCACAGAGGGTGATG |
| 249 | E51SL7 | CAAGGAAGATGGCATTTCTAGT | 257 | E51SL19 | AGTTATAAAATCACAGAGGGTG |
| 249 | E51SL7 | CAAGGAAGATGGCATTTCTAGT | 258 | E51SL20 | TGATCAAGTTATAAAATCACAG |
| 249 | E51SL7 | CAAGGAAGATGGCATTTCTAGT | 259 | E51SL21 | TTGATCAAGTTATAAAATCACA |
| 249 | E51SL7 | CAAGGAAGATGGCATTTCTAGT | 260 | E51SL22 | GGGCTTGGACAGAACTTACCGA |
| 249 | E51SL7 | CAAGGAAGATGGCATTTCTAGT | 261 | E51SL23 | CTCTGGCAGATTTCAACCGGGC |
| 249 | E51SL7 | CAAGGAAGATGGCATTTCTAGT | 262 | E51SL24 | ACCTGCTCTGGCAGATTTCAAC |
| 249 | E51SL7 | CAAGGAAGATGGCATTTCTAGT | 263 | E51SL25 | TACCTGCTCTGGCAGATTTCAA |
| 249 | E51SL7 | CAAGGAAGATGGCATTTCTAGT | 264 | E51SL26 | CTTGATGTTGGAGGTACCTGCT |
| 249 | E51SL7 | CAAGGAAGATGGCATTTCTAGT | 265 | E51SL27 | AATGCCATCTTCCTTGATGTTG |
| 249 | E51SL7 | CAAGGAAGATGGCATTTCTAGT | 266 | E51SL28 | AGAAATGCCATCTTCCTTGATG |
| 249 | E51SL7 | CAAGGAAGATGGCATTTCTAGT | 267 | E51SL29 | GGTGACACAACCTGTGGTTACT |
| 249 | E51SL7 | CAAGGAAGATGGCATTTCTAGT | 268 | E51SL30 | TGTTACTCTGGTGACACAACCT |
| 249 | E51SL7 | CAAGGAAGATGGCATTTCTAGT | 269 | E51SL31 | AGCTCCTACTCAGACTGTTACT |
| 250 | E51SL8 | AGATGGCATTTCTAGTTTGGAG | 251 | E51SL9 | ATGGCAGTTTCCTTAGTAACCA |
| 250 | E51SL8 | AGATGGCATTTCTAGTTTGGAG | 252 | E51SL10 | GTCACCAGAGTAACAGTCTGAG |
| 250 | E51SL8 | AGATGGCATTTCTAGTTTGGAG | 253 | E51SL15 | CAACGAGATGATCATCAAGCAG |
| 250 | E51SL8 | AGATGGCATTTCTAGTTTGGAG | 254 | E51SL16 | GAGGGTGATGGTGGGTGACCTT |
| 250 | E51SL8 | AGATGGCATTTCTAGTTTGGAG | 255 | E51SL17 | ATAAAATCACAGAGGGTGATGG |
| 250 | E51SL8 | AGATGGCATTTCTAGTTTGGAG | 256 | E51SL18 | TATAAAATCACAGAGGGTGATG |
| 250 | E51SL8 | AGATGGCATTTCTAGTTTGGAG | 257 | E51SL19 | AGTTATAAAATCACAGAGGGTG |
| 250 | E51SL8 | AGATGGCATTTCTAGTTTGGAG | 258 | E51SL20 | TGATCAAGTTATAAAATCACAG |
| 250 | E51SL8 | AGATGGCATTTCTAGTTTGGAG | 259 | E51SL21 | TTGATCAAGTTATAAAATCACA |
| 250 | E51SL8 | AGATGGCATTTCTAGTTTGGAG | 260 | E51SL22 | GGGCTTGGACAGAACTTACCGA |
| 250 | E51SL8 | AGATGGCATTTCTAGTTTGGAG | 261 | E51SL23 | CTCTGGCAGATTTCAACCGGGC |
| 250 | E51SL8 | AGATGGCATTTCTAGTTTGGAG | 262 | E51SL24 | ACCTGCTCTGGCAGATTTCAAC |
| 250 | E51SL8 | AGATGGCATTTCTAGTTTGGAG | 263 | E51SL25 | TACCTGCTCTGGCAGATTTCAA |
| 250 | E51SL8 | AGATGGCATTTCTAGTTTGGAG | 264 | E51SL26 | CTTGATGTTGGAGGTACCTGCT |
| 250 | E51SL8 | AGATGGCATTTCTAGTTTGGAG | 265 | E51SL27 | AATGCCATCTTCCTTGATGTTG |
| 250 | E51SL8 | AGATGGCATTTCTAGTTTGGAG | 266 | E51SL28 | AGAAATGCCATCTTCCTTGATG |
| 250 | E51SL8 | AGATGGCATTTCTAGTTTGGAG | 267 | E51SL29 | GGTGACACAACCTGTGGTTACT |
| 250 | E51SL8 | AGATGGCATTTCTAGTTTGGAG | 268 | E51SL30 | TGTTACTCTGGTGACACAACCT |
| 250 | E51SL8 | AGATGGCATTTCTAGTTTGGAG | 269 | E51SL31 | AGCTCCTACTCAGACTGTTACT |
| 251 | E51SL9 | ATGGCAGTTTCCTTAGTAACCA | 252 | E51SL10 | GTCACCAGAGTAACAGTCTGAG |
| 251 | E51SL9 | ATGGCAGTTTCCTTAGTAACCA | 253 | E51SL15 | CAACGAGATGATCATCAAGCAG |
| 251 | E51SL9 | ATGGCAGTTTCCTTAGTAACCA | 254 | E51SL16 | GAGGGTGATGGTGGGTGACCTT |
| 251 | E51SL9 | ATGGCAGTTTCCTTAGTAACCA | 255 | E51SL17 | ATAAAATCACAGAGGGTGATGG |
| 251 | E51SL9 | ATGGCAGTTTCCTTAGTAACCA | 256 | E51SL18 | TATAAAATCACAGAGGGTGATG |
| 251 | E51SL9 | ATGGCAGTTTCCTTAGTAACCA | 257 | E51SL19 | AGTTATAAAATCACAGAGGGTG |
| 251 | E51SL9 | ATGGCAGTTTCCTTAGTAACCA | 258 | E51SL20 | TGATCAAGTTATAAAATCACAG |
| 251 | E51SL9 | ATGGCAGTTTCCTTAGTAACCA | 259 | E51SL21 | TTGATCAAGTTATAAAATCACA |
| 251 | E51SL9 | ATGGCAGTTTCCTTAGTAACCA | 260 | E51SL22 | GGGCTTGGACAGAACTTACCGA |
| 251 | E51SL9 | ATGGCAGTTTCCTTAGTAACCA | 261 | E51SL23 | CTCTGGCAGATTTCAACCGGGC |
| 251 | E51SL9 | ATGGCAGTTTCCTTAGTAACCA | 262 | E51SL24 | ACCTGCTCTGGCAGATTTCAAC |
| 251 | E51SL9 | ATGGCAGTTTCCTTAGTAACCA | 263 | E51SL25 | TACCTGCTCTGGCAGATTTCAA |
| 251 | E51SL9 | ATGGCAGTTTCCTTAGTAACCA | 264 | E51SL26 | CTTGATGTTGGAGGTACCTGCT |
| 251 | E51SL9 | ATGGCAGTTTCCTTAGTAACCA | 265 | E51SL27 | AATGCCATCTTCCTTGATGTTG |
| 251 | E51SL9 | ATGGCAGTTTCCTTAGTAACCA | 266 | E51SL28 | AGAAATGCCATCTTCCTTGATG |
| 251 | E51SL9 | ATGGCAGTTTCCTTAGTAACCA | 267 | E51SL29 | GGTGACACAACCTGTGGTTACT |
| 251 | E51SL9 | ATGGCAGTTTCCTTAGTAACCA | 268 | E51SL30 | TGTTACTCTGGTGACACAACCT |
| 251 | E51SL9 | ATGGCAGTTTCCTTAGTAACCA | 269 | E51SL31 | AGCTCCTACTCAGACTGTTACT |
| 252 | E51SL10 | GTCACCAGAGTAACAGTCTGAG | 253 | E51SL15 | CAACGAGATGATCATCAAGCAG |
| 252 | E51SL10 | GTCACCAGAGTAACAGTCTGAG | 254 | E51SL16 | GAGGGTGATGGTGGGTGACCTT |
| 252 | E51SL10 | GTCACCAGAGTAACAGTCTGAG | 255 | E51SL17 | ATAAAATCACAGAGGGTGATGG |
| 252 | E51SL10 | GTCACCAGAGTAACAGTCTGAG | 256 | E51SL18 | TATAAAATCACAGAGGGTGATG |
| 252 | E51SL10 | GTCACCAGAGTAACAGTCTGAG | 257 | E51SL19 | AGTTATAAAATCACAGAGGGTG |
| 252 | E51SL10 | GTCACCAGAGTAACAGTCTGAG | 258 | E51SL20 | TGATCAAGTTATAAAATCACAG |
| 252 | E51SL10 | GTCACCAGAGTAACAGTCTGAG | 259 | E51SL21 | TTGATCAAGTTATAAAATCACA |
| 252 | E51SL10 | GTCACCAGAGTAACAGTCTGAG | 260 | E51SL22 | GGGCTTGGACAGAACTTACCGA |
| 252 | E51SL10 | GTCACCAGAGTAACAGTCTGAG | 261 | E51SL23 | CTCTGGCAGATTTCAACCGGGC |
| 252 | E51SL10 | GTCACCAGAGTAACAGTCTGAG | 262 | E51SL24 | ACCTGCTCTGGCAGATTTCAAC |
| 252 | E51SL10 | GTCACCAGAGTAACAGTCTGAG | 263 | E51SL25 | TACCTGCTCTGGCAGATTTCAA |
| 252 | E51SL10 | GTCACCAGAGTAACAGTCTGAG | 264 | E51SL26 | CTTGATGTTGGAGGTACCTGCT |
| 252 | E51SL10 | GTCACCAGAGTAACAGTCTGAG | 265 | E51SL27 | AATGCCATCTTCCTTGATGTTG |
| 252 | E51SL10 | GTCACCAGAGTAACAGTCTGAG | 266 | E51SL28 | AGAAATGCCATCTTCCTTGATG |
| 252 | E51SL10 | GTCACCAGAGTAACAGTCTGAG | 267 | E51SL29 | GGTGACACAACCTGTGGTTACT |
| 252 | E51SL10 | GTCACCAGAGTAACAGTCTGAG | 268 | E51SL30 | TGTTACTCTGGTGACACAACCT |
| 252 | E51SL10 | GTCACCAGAGTAACAGTCTGAG | 269 | E51SL31 | AGCTCCTACTCAGACTGTTACT |
| 253 | E51SL15 | CAACGAGATGATCATCAAGCAG | 254 | E51SL16 | GAGGGTGATGGTGGGTGACCTT |
| 253 | E51SL15 | CAACGAGATGATCATCAAGCAG | 255 | E51SL17 | ATAAAATCACAGAGGGTGATGG |
| 253 | E51SL15 | CAACGAGATGATCATCAAGCAG | 256 | E51SL18 | TATAAAATCACAGAGGGTGATG |
| 253 | E51SL15 | CAACGAGATGATCATCAAGCAG | 257 | E51SL19 | AGTTATAAAATCACAGAGGGTG |
| 253 | E51SL15 | CAACGAGATGATCATCAAGCAG | 258 | E51SL20 | TGATCAAGTTATAAAATCACAG |
| 253 | E51SL15 | CAACGAGATGATCATCAAGCAG | 259 | E51SL21 | TTGATCAAGTTATAAAATCACA |
| 253 | E51SL15 | CAACGAGATGATCATCAAGCAG | 260 | E51SL22 | GGGCTTGGACAGAACTTACCGA |
| 253 | E51SL15 | CAACGAGATGATCATCAAGCAG | 261 | E51SL23 | CTCTGGCAGATTTCAACCGGGC |
| 253 | E51SL15 | CAACGAGATGATCATCAAGCAG | 262 | E51SL24 | ACCTGCTCTGGCAGATTTCAAC |
| 253 | E51SL15 | CAACGAGATGATCATCAAGCAG | 263 | E51SL25 | TACCTGCTCTGGCAGATTTCAA |
| 253 | E51SL15 | CAACGAGATGATCATCAAGCAG | 264 | E51SL26 | CTTGATGTTGGAGGTACCTGCT |
| 253 | E51SL15 | CAACGAGATGATCATCAAGCAG | 265 | E51SL27 | AATGCCATCTTCCTTGATGTTG |
| 253 | E51SL15 | CAACGAGATGATCATCAAGCAG | 266 | E51SL28 | AGAAATGCCATCTTCCTTGATG |
| 253 | E51SL15 | CAACGAGATGATCATCAAGCAG | 267 | E51SL29 | GGTGACACAACCTGTGGTTACT |
| 253 | E51SL15 | CAACGAGATGATCATCAAGCAG | 268 | E51SL30 | TGTTACTCTGGTGACACAACCT |
| 253 | E51SL15 | CAACGAGATGATCATCAAGCAG | 269 | E51SL31 | AGCTCCTACTCAGACTGTTACT |
| 254 | E51SL16 | GAGGGTGATGGTGGGTGACCTT | 255 | E51SL17 | ATAAAATCACAGAGGGTGATGG |
| 254 | E51SL16 | GAGGGTGATGGTGGGTGACCTT | 256 | E51SL18 | TATAAAATCACAGAGGGTGATG |
| 254 | E51SL16 | GAGGGTGATGGTGGGTGACCTT | 257 | E51SL19 | AGTTATAAAATCACAGAGGGTG |
| 254 | E51SL16 | GAGGGTGATGGTGGGTGACCTT | 258 | E51SL20 | TGATCAAGTTATAAAATCACAG |
| 254 | E51SL16 | GAGGGTGATGGTGGGTGACCTT | 259 | E51SL21 | TTGATCAAGTTATAAAATCACA |
| 254 | E51SL16 | GAGGGTGATGGTGGGTGACCTT | 260 | E51SL22 | GGGCTTGGACAGAACTTACCGA |
| 254 | E51SL16 | GAGGGTGATGGTGGGTGACCTT | 261 | E51SL23 | CTCTGGCAGATTTCAACCGGGC |
| 254 | E51SL16 | GAGGGTGATGGTGGGTGACCTT | 262 | E51SL24 | ACCTGCTCTGGCAGATTTCAAC |
| 254 | E51SL16 | GAGGGTGATGGTGGGTGACCTT | 263 | E51SL25 | TACCTGCTCTGGCAGATTTCAA |
| 254 | E51SL16 | GAGGGTGATGGTGGGTGACCTT | 264 | E51SL26 | CTTGATGTTGGAGGTACCTGCT |
| 254 | E51SL16 | GAGGGTGATGGTGGGTGACCTT | 265 | E51SL27 | AATGCCATCTTCCTTGATGTTG |
| 254 | E51SL16 | GAGGGTGATGGTGGGTGACCTT | 266 | E51SL28 | AGAAATGCCATCTTCCTTGATG |
| 254 | E51SL16 | GAGGGTGATGGTGGGTGACCTT | 267 | E51SL29 | GGTGACACAACCTGTGGTTACT |
| 254 | E51SL16 | GAGGGTGATGGTGGGTGACCTT | 268 | E51SL30 | TGTTACTCTGGTGACACAACCT |
| 254 | E51SL16 | GAGGGTGATGGTGGGTGACCTT | 269 | E51SL31 | AGCTCCTACTCAGACTGTTACT |
| 255 | E51SL17 | ATAAAATCACAGAGGGTGATGG | 256 | E51SL18 | TATAAAATCACAGAGGGTGATG |
| 255 | E51SL17 | ATAAAATCACAGAGGGTGATGG | 257 | E51SL19 | AGTTATAAAATCACAGAGGGTG |
| 255 | E51SL17 | ATAAAATCACAGAGGGTGATGG | 258 | E51SL20 | TGATCAAGTTATAAAATCACAG |
| 255 | E51SL17 | ATAAAATCACAGAGGGTGATGG | 259 | E51SL21 | TTGATCAAGTTATAAAATCACA |
| 255 | E51SL17 | ATAAAATCACAGAGGGTGATGG | 260 | E51SL22 | GGGCTTGGACAGAACTTACCGA |
| 255 | E51SL17 | ATAAAATCACAGAGGGTGATGG | 261 | E51SL23 | CTCTGGCAGATTTCAACCGGGC |
| 255 | E51SL17 | ATAAAATCACAGAGGGTGATGG | 262 | E51SL24 | ACCTGCTCTGGCAGATTTCAAC |
| 255 | E51SL17 | ATAAAATCACAGAGGGTGATGG | 263 | E51SL25 | TACCTGCTCTGGCAGATTTCAA |
| 255 | E51SL17 | ATAAAATCACAGAGGGTGATGG | 264 | E51SL26 | CTTGATGTTGGAGGTACCTGCT |
| 255 | E51SL17 | ATAAAATCACAGAGGGTGATGG | 265 | E51SL27 | AATGCCATCTTCCTTGATGTTG |
| 255 | E51SL17 | ATAAAATCACAGAGGGTGATGG | 266 | E51SL28 | AGAAATGCCATCTTCCTTGATG |
| 255 | E51SL17 | ATAAAATCACAGAGGGTGATGG | 267 | E51SL29 | GGTGACACAACCTGTGGTTACT |
| 255 | E51SL17 | ATAAAATCACAGAGGGTGATGG | 268 | E51SL30 | TGTTACTCTGGTGACACAACCT |
| 255 | E51SL17 | ATAAAATCACAGAGGGTGATGG | 269 | E51SL31 | AGCTCCTACTCAGACTGTTACT |
| 256 | E51SL18 | TATAAAATCACAGAGGGTGATG | 257 | E51SL19 | AGTTATAAAATCACAGAGGGTG |
| 256 | E51SL18 | TATAAAATCACAGAGGGTGATG | 258 | E51SL20 | TGATCAAGTTATAAAATCACAG |
| 256 | E51SL18 | TATAAAATCACAGAGGGTGATG | 259 | E51SL21 | TTGATCAAGTTATAAAATCACA |
| 256 | E51SL18 | TATAAAATCACAGAGGGTGATG | 260 | E51SL22 | GGGCTTGGACAGAACTTACCGA |
| 256 | E51SL18 | TATAAAATCACAGAGGGTGATG | 261 | E51SL23 | CTCTGGCAGATTTCAACCGGGC |
| 256 | E51SL18 | TATAAAATCACAGAGGGTGATG | 262 | E51SL24 | ACCTGCTCTGGCAGATTTCAAC |
| 256 | E51SL18 | TATAAAATCACAGAGGGTGATG | 263 | E51SL25 | TACCTGCTCTGGCAGATTTCAA |
| 256 | E51SL18 | TATAAAATCACAGAGGGTGATG | 264 | E51SL26 | CTTGATGTTGGAGGTACCTGCT |
| 256 | E51SL18 | TATAAAATCACAGAGGGTGATG | 265 | E51SL27 | AATGCCATCTTCCTTGATGTTG |
| 256 | E51SL18 | TATAAAATCACAGAGGGTGATG | 266 | E51SL28 | AGAAATGCCATCTTCCTTGATG |
| 256 | E51SL18 | TATAAAATCACAGAGGGTGATG | 267 | E51SL29 | GGTGACACAACCTGTGGTTACT |
| 256 | E51SL18 | TATAAAATCACAGAGGGTGATG | 268 | E51SL30 | TGTTACTCTGGTGACACAACCT |
| 256 | E51SL18 | TATAAAATCACAGAGGGTGATG | 269 | E51SL31 | AGCTCCTACTCAGACTGTTACT |
| 257 | E51SL19 | AGTTATAAAATCACAGAGGGTG | 258 | E51SL20 | TGATCAAGTTATAAAATCACAG |
| 257 | E51SL19 | AGTTATAAAATCACAGAGGGTG | 259 | E51SL21 | TTGATCAAGTTATAAAATCACA |
| 257 | E51SL19 | AGTTATAAAATCACAGAGGGTG | 260 | E51SL22 | GGGCTTGGACAGAACTTACCGA |
| 257 | E51SL19 | AGTTATAAAATCACAGAGGGTG | 261 | E51SL23 | CTCTGGCAGATTTCAACCGGGC |
| 257 | E51SL19 | AGTTATAAAATCACAGAGGGTG | 262 | E51SL24 | ACCTGCTCTGGCAGATTTCAAC |
| 257 | E51SL19 | AGTTATAAAATCACAGAGGGTG | 263 | E51SL25 | TACCTGCTCTGGCAGATTTCAA |
| 257 | E51SL19 | AGTTATAAAATCACAGAGGGTG | 264 | E51SL26 | CTTGATGTTGGAGGTACCTGCT |
| 257 | E51SL19 | AGTTATAAAATCACAGAGGGTG | 265 | E51SL27 | AATGCCATCTTCCTTGATGTTG |
| 257 | E51SL19 | AGTTATAAAATCACAGAGGGTG | 266 | E51SL28 | AGAAATGCCATCTTCCTTGATG |
| 257 | E51SL19 | AGTTATAAAATCACAGAGGGTG | 267 | E51SL29 | GGTGACACAACCTGTGGTTACT |
| 257 | E51SL19 | AGTTATAAAATCACAGAGGGTG | 268 | E51SL30 | TGTTACTCTGGTGACACAACCT |
| 257 | E51SL19 | AGTTATAAAATCACAGAGGGTG | 269 | E51SL31 | AGCTCCTACTCAGACTGTTACT |
| 258 | E51SL20 | TGATCAAGTTATAAAATCACAG | 259 | E51SL21 | TTGATCAAGTTATAAAATCACA |
| 258 | E51SL20 | TGATCAAGTTATAAAATCACAG | 260 | E51SL22 | GGGCTTGGACAGAACTTACCGA |
| 258 | E51SL20 | TGATCAAGTTATAAAATCACAG | 261 | E51SL23 | CTCTGGCAGATTTCAACCGGGC |
| 258 | E51SL20 | TGATCAAGTTATAAAATCACAG | 262 | E51SL24 | ACCTGCTCTGGCAGATTTCAAC |
| 258 | E51SL20 | TGATCAAGTTATAAAATCACAG | 263 | E51SL25 | TACCTGCTCTGGCAGATTTCAA |
| 258 | E51SL20 | TGATCAAGTTATAAAATCACAG | 264 | E51SL26 | CTTGATGTTGGAGGTACCTGCT |
| 258 | E51SL20 | TGATCAAGTTATAAAATCACAG | 265 | E51SL27 | AATGCCATCTTCCTTGATGTTG |
| 258 | E51SL20 | TGATCAAGTTATAAAATCACAG | 266 | E51SL28 | AGAAATGCCATCTTCCTTGATG |
| 258 | E51SL20 | TGATCAAGTTATAAAATCACAG | 267 | E51SL29 | GGTGACACAACCTGTGGTTACT |
| 258 | E51SL20 | TGATCAAGTTATAAAATCACAG | 268 | E51SL30 | TGTTACTCTGGTGACACAACCT |
| 258 | E51SL20 | TGATCAAGTTATAAAATCACAG | 269 | E51SL31 | AGCTCCTACTCAGACTGTTACT |
| 259 | E51SL21 | TTGATCAAGTTATAAAATCACA | 260 | E51SL22 | GGGCTTGGACAGAACTTACCGA |
| 259 | E51SL21 | TTGATCAAGTTATAAAATCACA | 261 | E51SL23 | CTCTGGCAGATTTCAACCGGGC |
| 259 | E51SL21 | TTGATCAAGTTATAAAATCACA | 262 | E51SL24 | ACCTGCTCTGGCAGATTTCAAC |
| 259 | E51SL21 | TTGATCAAGTTATAAAATCACA | 263 | E51SL25 | TACCTGCTCTGGCAGATTTCAA |
| 259 | E51SL21 | TTGATCAAGTTATAAAATCACA | 264 | E51SL26 | CTTGATGTTGGAGGTACCTGCT |
| 259 | E51SL21 | TTGATCAAGTTATAAAATCACA | 265 | E51SL27 | AATGCCATCTTCCTTGATGTTG |
| 259 | E51SL21 | TTGATCAAGTTATAAAATCACA | 266 | E51SL28 | AGAAATGCCATCTTCCTTGATG |
| 259 | E51SL21 | TTGATCAAGTTATAAAATCACA | 267 | E51SL29 | GGTGACACAACCTGTGGTTACT |
| 259 | E51SL21 | TTGATCAAGTTATAAAATCACA | 268 | E51SL30 | TGTTACTCTGGTGACACAACCT |
| 259 | E51SL21 | TTGATCAAGTTATAAAATCACA | 269 | E51SL31 | AGCTCCTACTCAGACTGTTACT |
| 260 | E51SL22 | GGGCTTGGACAGAACTTACCGA | 261 | E51SL23 | CTCTGGCAGATTTCAACCGGGC |
| 260 | E51SL22 | GGGCTTGGACAGAACTTACCGA | 262 | E51SL24 | ACCTGCTCTGGCAGATTTCAAC |
| 260 | E51SL22 | GGGCTTGGACAGAACTTACCGA | 263 | E51SL25 | TACCTGCTCTGGCAGATTTCAA |
| 260 | E51SL22 | GGGCTTGGACAGAACTTACCGA | 264 | E51SL26 | CTTGATGTTGGAGGTACCTGCT |
| 260 | E51SL22 | GGGCTTGGACAGAACTTACCGA | 265 | E51SL27 | AATGCCATCTTCCTTGATGTTG |
| 260 | E51SL22 | GGGCTTGGACAGAACTTACCGA | 266 | E51SL28 | AGAAATGCCATCTTCCTTGATG |
| 260 | E51SL22 | GGGCTTGGACAGAACTTACCGA | 267 | E51SL29 | GGTGACACAACCTGTGGTTACT |
| 260 | E51SL22 | GGGCTTGGACAGAACTTACCGA | 268 | E51SL30 | TGTTACTCTGGTGACACAACCT |
| 260 | E51SL22 | GGGCTTGGACAGAACTTACCGA | 269 | E51SL31 | AGCTCCTACTCAGACTGTTACT |
| 261 | E51SL23 | CTCTGGCAGATTTCAACCGGGC | 262 | E51SL24 | ACCTGCTCTGGCAGATTTCAAC |
| 261 | E51SL23 | CTCTGGCAGATTTCAACCGGGC | 263 | E51SL25 | TACCTGCTCTGGCAGATTTCAA |
| 261 | E51SL23 | CTCTGGCAGATTTCAACCGGGC | 264 | E51SL26 | CTTGATGTTGGAGGTACCTGCT |
| 261 | E51SL23 | CTCTGGCAGATTTCAACCGGGC | 265 | E51SL27 | AATGCCATCTTCCTTGATGTTG |
| 261 | E51SL23 | CTCTGGCAGATTTCAACCGGGC | 266 | E51SL28 | AGAAATGCCATCTTCCTTGATG |
| 261 | E51SL23 | CTCTGGCAGATTTCAACCGGGC | 267 | E51SL29 | GGTGACACAACCTGTGGTTACT |
| 261 | E51SL23 | CTCTGGCAGATTTCAACCGGGC | 268 | E51SL30 | TGTTACTCTGGTGACACAACCT |
| 261 | E51SL23 | CTCTGGCAGATTTCAACCGGGC | 269 | E51SL31 | AGCTCCTACTCAGACTGTTACT |
| 262 | E51SL24 | ACCTGCTCTGGCAGATTTCAAC | 263 | E51SL25 | TACCTGCTCTGGCAGATTTCAA |
| 262 | E51SL24 | ACCTGCTCTGGCAGATTTCAAC | 264 | E51SL26 | CTTGATGTTGGAGGTACCTGCT |
| 262 | E51SL24 | ACCTGCTCTGGCAGATTTCAAC | 265 | E51SL27 | AATGCCATCTTCCTTGATGTTG |
| 262 | E51SL24 | ACCTGCTCTGGCAGATTTCAAC | 266 | E51SL28 | AGAAATGCCATCTTCCTTGATG |
| 262 | E51SL24 | ACCTGCTCTGGCAGATTTCAAC | 267 | E51SL29 | GGTGACACAACCTGTGGTTACT |
| 262 | E51SL24 | ACCTGCTCTGGCAGATTTCAAC | 268 | E51SL30 | TGTTACTCTGGTGACACAACCT |
| 262 | E51SL24 | ACCTGCTCTGGCAGATTTCAAC | 269 | E51SL31 | AGCTCCTACTCAGACTGTTACT |
| 263 | E51SL25 | TACCTGCTCTGGCAGATTTCAA | 264 | E51SL26 | CTTGATGTTGGAGGTACCTGCT |
| 263 | E51SL25 | TACCTGCTCTGGCAGATTTCAA | 265 | E51SL27 | AATGCCATCTTCCTTGATGTTG |
| 263 | E51SL25 | TACCTGCTCTGGCAGATTTCAA | 266 | E51SL28 | AGAAATGCCATCTTCCTTGATG |
| 263 | E51SL25 | TACCTGCTCTGGCAGATTTCAA | 267 | E51SL29 | GGTGACACAACCTGTGGTTACT |
| 263 | E51SL25 | TACCTGCTCTGGCAGATTTCAA | 268 | E51SL30 | TGTTACTCTGGTGACACAACCT |
| 263 | E51SL25 | TACCTGCTCTGGCAGATTTCAA | 269 | E51SL31 | AGCTCCTACTCAGACTGTTACT |
| 264 | E51SL26 | CTTGATGTTGGAGGTACCTGCT | 265 | E51SL27 | AATGCCATCTTCCTTGATGTTG |
| 264 | E51SL26 | CTTGATGTTGGAGGTACCTGCT | 266 | E51SL28 | AGAAATGCCATCTTCCTTGATG |
| 264 | E51SL26 | CTTGATGTTGGAGGTACCTGCT | 267 | E51SL29 | GGTGACACAACCTGTGGTTACT |
| 264 | E51SL26 | CTTGATGTTGGAGGTACCTGCT | 268 | E51SL30 | TGTTACTCTGGTGACACAACCT |
| 264 | E51SL26 | CTTGATGTTGGAGGTACCTGCT | 269 | E51SL31 | AGCTCCTACTCAGACTGTTACT |
| 265 | E51SL27 | AATGCCATCTTCCTTGATGTTG | 266 | E51SL28 | AGAAATGCCATCTTCCTTGATG |
| 265 | E51SL27 | AATGCCATCTTCCTTGATGTTG | 267 | E51SL29 | GGTGACACAACCTGTGGTTACT |
| 265 | E51SL27 | AATGCCATCTTCCTTGATGTTG | 268 | E51SL30 | TGTTACTCTGGTGACACAACCT |
| 265 | E51SL27 | AATGCCATCTTCCTTGATGTTG | 269 | E51SL31 | AGCTCCTACTCAGACTGTTACT |
| 266 | E51SL28 | AGAAATGCCATCTTCCTTGATG | 267 | E51SL29 | GGTGACACAACCTGTGGTTACT |
| 266 | E51SL28 | AGAAATGCCATCTTCCTTGATG | 268 | E51SL30 | TGTTACTCTGGTGACACAACCT |
| 266 | E51SL28 | AGAAATGCCATCTTCCTTGATG | 269 | E51SL31 | AGCTCCTACTCAGACTGTTACT |
| 267 | E51SL29 | GGTGACACAACCTGTGGTTACT | 268 | E51SL30 | TGTTACTCTGGTGACACAACCT |
| 267 | E51SL29 | GGTGACACAACCTGTGGTTACT | 269 | E51SL31 | AGCTCCTACTCAGACTGTTACT |
| 268 | E51SL30 | TGTTACTCTGGTGACACAACCT | 269 | E51SL31 | AGCTCCTACTCAGACTGTTACT |

### e) Exon 53:

TABLE 5A
(1). Exon 53 SaCas9 guides:

| Seq | Guide ID or | |
|---|---|---|
| ID No. | Guide RNA Name | Guide Sequence |
| 16 | E53Sa1 | CCTTGGTTTCTGTGATTTTCTT |
| 17 | E53Sa2 | TCCTTAGCTTCCAGCCATTGTG |
| 18 | E53Sa3 | CTTGTACTTCATCCCACTGATT |
| 19 | E53Sa4 | ACTGATTCTGAATTCTTTCAAC |
| 20 | E53Sa5 | AGCCAAGCTTGAGTCATGGAAG |
| 21 | E53Sa6 | TTAGGACAGGCCAGAGCCAAGC |
| 22 | E53Sa7 | GCAACAGTTGAATGAAATGTTA |
| 23 | E53Sa8 | CCTTCAGAACCGGAGGCAACAG |
| 24 | E53Sa9 | AGTTGAAAGAATTCAGAATCAG |
| 25 | E53Sa10 | TTTTATTCTAGTTGAAAGAATT |
| 26 | E53Sa11 | TTTTTCCTTTTATTCTAGTTGA |
| 70 | E53SaCas9KKH1 | AAAAGGTATCTTTGATACTAAC |
| 71 | E53SaCas9KKH2 | CTTTGATACTAACCTTGGTTTC |
| 72 | E53SaCas9KKH3 | CCTTGGTTTCTGTGATTTTCTT |
| 73 | E53SaCas9KKH4 | TCCTTAGCTTCCAGCCATTGTG |
| 74 | E53SaCas9KKH5 | AACATTTCATTCAACTGTTGCC |
| 75 | E53SaCas9KKH6 | TTCAACTGTTGCCTCCGGTTCT |
| 76 | E53SaCas9KKH7 | AGGTGTTCTTGTACTTCATCCC |
| 77 | E53SaCas9KKH8 | CTTGTACTTCATCCCACTGATT |
| 78 | E53SaCas9KKH9 | ACTGATTCTGAATTCTTTCAAC |
| 79 | E53SaCas9KKH10 | TTCTTTCAACTAGAATAAAAGG |
| 80 | E53SaCas9KKH11 | TTCAACTAGAATAAAAGGAAAA |
| 81 | E53SaCas9KKH12 | AGAATAAAAGGAAAAATAAATA |
| 82 | E53SaCas9KKH13 | GTTAGTATCAAAGATACCTTTT |
| 83 | E53SaCas9KKH14 | CACAGAAACCAAGGTTAGTATC |
| 84 | E53SaCas9KKH15 | AAAGAAAATCACAGAAACCAAG |
| 85 | E53SaCas9KKH16 | TCCAAAAGAAAATCACAGAAAC |
| 86 | E53SaCas9KKH17 | ATACAGTAGATGCAATCCAAAA |
| 87 | E53SaCas9KKH18 | AGGAGGGTCCCTATACAGTAGA |
| 88 | E53SaCas9KKH19 | ATGGAAGGAGGGTCCCTATACA |
| 89 | E53SaCas9KKH20 | AGTCATGGAAGGAGGGTCCCTA |
| 90 | E53SaCas9KKH21 | AGCCAAGCTTGAGTCATGGAAG |
| 91 | E53SaCas9KKH22 | TTAGGACAGGCCAGAGCCAAGC |
| 92 | E53SaCas9KKH23 | TGGAAGCTAAGGAAGAAGCTGA |
| 93 | E53SaCas9KKH24 | AATGAAATGTTAAAGGATTCAA |
| 94 | E53SaCas9KKH25 | GCAACAGTTGAATGAAATGTTA |
| 95 | E53SaCas9KKH26 | AGAACCGGAGGCAACAGTTGAA |
| 96 | E53SaCas9KKH27 | CCTTCAGAACCGGAGGCAACAG |
| 97 | E53SaCas9KKH28 | GAACACCTTCAGAACCGGAGGC |
| 98 | E53SaCas9KKH29 | AAAGAATTCAGAATCAGTGGGA |
| 99 | E53SaCas9KKH30 | AGTTGAAAGAATTCAGAATCAG |
| 100 | E53SaCas9KKH31 | ATTCTAGTTGAAAGAATTCAGA |
| 101 | E53SaCas9KKH32 | TTTTATTCTAGTTGAAAGAATT |
| 102 | E53SaCas9KKH33 | TTTTTCCTTTTATTCTAGTTGA |
| 103 | E53SaCas9KKH34 | ATATATTTATTTTTCCTTTTAT |

TABLE 5B
(2). Exon 53 SaCas9 pairs:

| Guide 1 | | | Guide 2 | | |
|---|---|---|---|---|---|
| Seq ID | | | Seq ID | | |
| No. | Guide 1 | Guide 1 Sequence | No. | Guide 2 | Guide 2 Sequence |
| 16 | E53Sa1 | CCTTGGTTTCTGTGATTTT | 17 | E53Sa2 | TCCTTAGCTTCCAGCCATT |
| | | CTT | | | GTG |
| 16 | E53Sa1 | CCTTGGTTTCTGTGATTTT | 18 | E53Sa3 | CTTGTACTTCATCCCACTG |
| | | CTT | | | ATT |
| 16 | E53Sa1 | CCTTGGTTTCTGTGATTTT | 19 | E53Sa4 | ACTGATTCTGAATTCTTTC |
| | | CTT | | | AAC |
| 16 | E53Sa1 | CCTTGGTTTCTGTGATTTT | 20 | E53Sa5 | AGCCAAGCTTGAGTCATGG |
| | | CTT | | | AAG |
| 16 | E53Sa1 | CCTTGGTTTCTGTGATTTT | 21 | E53Sa6 | TTAGGACAGGCCAGAGCCA |
| | | CTT | | | AGC |
| 16 | E53Sa1 | CCTTGGTTTCTGTGATTTT | 22 | E53Sa7 | GCAACAGTTGAATGAAATG |
| | | CTT | | | TTA |
| 16 | E53Sa1 | CCTTGGTTTCTGTGATTTT | 23 | E53Sa8 | CCTTCAGAACCGGAGGCAA |
| | | CTT | | | CAG |
| 16 | E53Sa1 | CCTTGGTTTCTGTGATTTT | 24 | E53Sa9 | AGTTGAAAGAATTCAGAAT |
| | | CTT | | | CAG |
| 16 | E53Sa1 | CCTTGGTTTCTGTGATTTT | 25 | E53Sa10 | TTTTATTCTAGTTGAAAGA |
| | | CTT | | | ATT |
| 16 | E53Sa1 | CCTTGGTTTCTGTGATTTT | 26 | E53Sa11 | TTTTTCCTTTTATTCTAGT |
| | | CTT | | | TGA |
| 17 | E53Sa2 | TCCTTAGCTTCCAGCCATT | 18 | E53Sa3 | CTTGTACTTCATCCCACTG |
| | | GTG | | | ATT |
| 17 | E53Sa2 | TCCTTAGCTTCCAGCCATT | 19 | E53Sa4 | ACTGATTCTGAATTCTTTC |
| | | GTG | | | AAC |
| 17 | E53Sa2 | TCCTTAGCTTCCAGCCATT | 20 | E53Sa5 | AGCCAAGCTTGAGTCATGG |
| | | GTG | | | AAG |
| 17 | E53Sa2 | TCCTTAGCTTCCAGCCATT | 21 | E53Sa6 | TTAGGACAGGCCAGAGCCA |
| | | GTG | | | AGC |
| 17 | E53Sa2 | TCCTTAGCTTCCAGCCATT | 22 | E53Sa7 | GCAACAGTTGAATGAAATG |
| | | GTG | | | TTA |
| 17 | E53Sa2 | TCCTTAGCTTCCAGCCATT | 23 | E53Sa8 | CCTTCAGAACCGGAGGCAA |
| | | GTG | | | CAG |
| 17 | E53Sa2 | TCCTTAGCTTCCAGCCATT | 24 | E53Sa9 | AGTTGAAAGAATTCAGAAT |
| | | GTG | | | CAG |
| 17 | E53Sa2 | TCCTTAGCTTCCAGCCATT | 25 | E53Sa10 | TTTTATTCTAGTTGAAAGA |
| | | GTG | | | ATT |
| 17 | E53Sa2 | TCCTTAGCTTCCAGCCATT | 26 | E53Sa11 | TTTTTCCTTTTATTCTAGT |
| | | GTG | | | TGA |
| 18 | E53Sa3 | CTTGTACTTCATCCCACTG | 19 | E53Sa4 | ACTGATTCTGAATTCTTTC |
| | | ATT | | | AAC |
| 18 | E53Sa3 | CTTGTACTTCATCCCACTG | 20 | E53Sa5 | AGCCAAGCTTGAGTCATGG |
| | | ATT | | | AAG |
| 18 | E53Sa3 | CTTGTACTTCATCCCACTG | 21 | E53Sa6 | TTAGGACAGGCCAGAGCCA |
| | | ATT | | | AGC |
| 18 | E53Sa3 | CTTGTACTTCATCCCACTG | 22 | E53Sa7 | GCAACAGTTGAATGAAATG |
| | | ATT | | | TTA |
| 18 | E53Sa3 | CTTGTACTTCATCCCACTG | 23 | E53Sa8 | CCTTCAGAACCGGAGGCAA |
| | | ATT | | | CAG |
| 18 | E53Sa3 | CTTGTACTTCATCCCACTG | 24 | E53Sa9 | AGTTGAAAGAATTCAGAAT |
| | | ATT | | | CAG |
| 18 | E53Sa3 | CTTGTACTTCATCCCACTG | 25 | E53Sa10 | TTTTATTCTAGTTGAAAGA |
| | | ATT | | | ATT |
| 18 | E53Sa3 | CTTGTACTTCATCCCACTG | 26 | E53Sa11 | TTTTTCCTTTTATTCTAGT |
| | | ATT | | | TGA |
| 19 | E53Sa4 | ACTGATTCTGAATTCTTTC | 20 | E53Sa5 | AGCCAAGCTTGAGTCATGG |
| | | AAC | | | AAG |
| 19 | E53Sa4 | ACTGATTCTGAATTCTTTC | 21 | E53Sa6 | TTAGGACAGGCCAGAGCCA |
| | | AAC | | | AGC |
| 19 | E53Sa4 | ACTGATTCTGAATTCTTTC | 22 | E53Sa7 | GCAACAGTTGAATGAAATG |
| | | AAC | | | TTA |
| 19 | E53Sa4 | ACTGATTCTGAATTCTTTC | 23 | E53Sa8 | CCTTCAGAACCGGAGGCAA |
| | | AAC | | | CAG |
| 19 | E53Sa4 | ACTGATTCTGAATTCTTTC | 24 | E53Sa9 | AGTTGAAAGAATTCAGAAT |
| | | AAC | | | CAG |
| 19 | E53Sa4 | ACTGATTCTGAATTCTTTC | 25 | E53Sa10 | TTTTATTCTAGTTGAAAGA |
| | | AAC | | | ATT |
| 19 | E53Sa4 | ACTGATTCTGAATTCTTTC | 26 | E53Sa11 | TTTTTCCTTTTATTCTAGT |
| | | AAC | | | TGA |
| 20 | E53Sa5 | AGCCAAGCTTGAGTCATGG | 21 | E53Sa6 | TTAGGACAGGCCAGAGCCA |
| | | AAG | | | AGC |
| 20 | E53Sa5 | AGCCAAGCTTGAGTCATGG | 22 | E53Sa7 | GCAACAGTTGAATGAAATG |
| | | AAG | | | TTA |
| 20 | E53Sa5 | AGCCAAGCTTGAGTCATGG | 23 | E53Sa8 | CCTTCAGAACCGGAGGCAA |
| | | AAG | | | CAG |
| 20 | E53Sa5 | AGCCAAGCTTGAGTCATGG | 24 | E53Sa9 | AGTTGAAAGAATTCAGAAT |
| | | AAG | | | CAG |
| 20 | E53Sa5 | AGCCAAGCTTGAGTCATGG | 25 | E53Sa10 | TTTTATTCTAGTTGAAAGA |
| | | AAG | | | ATT |
| 20 | E53Sa5 | AGCCAAGCTTGAGTCATGG | 26 | E53Sa11 | TTTTTCCTTTTATTCTAGT |
| | | AAG | | | TGA |
| 21 | E53Sa6 | TTAGGACAGGCCAGAGCCA | 22 | E53Sa7 | GCAACAGTTGAATGAAATG |
| | | AGC | | | TTA |
| 21 | E53Sa6 | TTAGGACAGGCCAGAGCCA | 23 | E53Sa8 | CCTTCAGAACCGGAGGCAA |
| | | AGC | | | CAG |
| 21 | E53Sa6 | TTAGGACAGGCCAGAGCCA | 24 | E53Sa9 | AGTTGAAAGAATTCAGAAT |
| | | AGC | | | CAG |
| 21 | E53Sa6 | TTAGGACAGGCCAGAGCCA | 25 | E53Sa10 | TTTTATTCTAGTTGAAAGA |
| | | AGC | | | ATT |
| 21 | E53Sa6 | TTAGGACAGGCCAGAGCCA | 26 | E53Sa11 | TTTTTCCTTTTATTCTAGT |
| | | AGC | | | TGA |
| 22 | E53Sa7 | GCAACAGTTGAATGAAATG | 23 | E53Sa8 | CCTTCAGAACCGGAGGCAA |
| | | TTA | | | CAG |
| 22 | E53Sa7 | GCAACAGTTGAATGAAATG | 24 | E53Sa9 | AGTTGAAAGAATTCAGAAT |
| | | TTA | | | CAG |
| 22 | E53Sa7 | GCAACAGTTGAATGAAATG | 25 | E53Sa10 | TTTTATTCTAGTTGAAAGA |
| | | TTA | | | ATT |
| 22 | E53Sa7 | GCAACAGTTGAATGAAATG | 26 | E53Sa11 | TTTTTCCTTTTATTCTAGT |
| | | TTA | | | TGA |
| 23 | E53Sa8 | CCTTCAGAACCGGAGGCAA | 24 | E53Sa9 | AGTTGAAAGAATTCAGAAT |
| | | CAG | | | CAG |
| 23 | E53Sa8 | CCTTCAGAACCGGAGGCAA | 25 | E53Sa10 | TTTTATTCTAGTTGAAAGA |
| | | CAG | | | ATT |
| 23 | E53Sa8 | CCTTCAGAACCGGAGGCAA | 26 | E53Sa11 | TTTTTCCTTTTATTCTAGT |
| | | CAG | | | TGA |
| 24 | E53Sa9 | AGTTGAAAGAATTCAGAAT | 25 | E53Sa10 | TTTTATTCTAGTTGAAAGA |
| | | CAG | | | ATT |
| 24 | E53Sa9 | AGTTGAAAGAATTCAGAAT | 26 | E53Sa11 | TTTTTCCTTTTATTCTAGT |
| | | CAG | | | TGA |
| 25 | E53Sa10 | TTTTATTCTAGTTGAAAGA | 26 | E53Sa11 | TTTTTCCTTTTATTCTAGT |
| | | ATT | | | TGA |
| 71 | E53SaCas9KKH2 | CTTTGATACTAACCTTGGT | 72 | E53SaCas9KKH3 | CCTTGGTTTCTGTGATTTT |
| | | TTC | | | CTT |
| 71 | E53SaCas9KKH2 | CTTTGATACTAACCTTGGT | 73 | E53SaCas9KKH4 | TCCTTAGCTTCCAGCCATT |
| | | TTC | | | GTG |
| 71 | E53SaCas9KKH2 | CTTTGATACTAACCTTGGT | 74 | E53SaCas9KKH5 | AACATTTCATTCAACTGTT |
| | | TTC | | | GCC |
| 71 | E53SaCas9KKH2 | CTTTGATACTAACCTTGGT | 75 | E53SaCas9KKH6 | TTCAACTGTTGCCTCCGGT |
| | | TTC | | | TCT |
| 71 | E53SaCas9KKH2 | CTTTGATACTAACCTTGGT | 76 | E53SaCas9KKH7 | AGGTGTTCTTGTACTTCAT |
| | | TTC | | | CCC |
| 71 | E53SaCas9KKH2 | CTTTGATACTAACCTTGGT | 77 | E53SaCas9KKH8 | CTTGTACTTCATCCCACTG |
| | | TTC | | | ATT |
| 71 | E53SaCas9KKH2 | CTTTGATACTAACCTTGGT | 78 | E53SaCas9KKH9 | ACTGATTCTGAATTCTTTC |
| | | TTC | | | AAC |
| 71 | E53SaCas9KKH2 | CTTTGATACTAACCTTGGT | 84 | E53SaCas9KKH15 | AAAGAAAATCACAGAAACC |
| | | TTC | | | AAG |
| 71 | E53SaCas9KKH2 | CTTTGATACTAACCTTGGT | 85 | E53SaCas9KKH16 | TCCAAAAGAAAATCACAGA |
| | | TTC | | | AAC |
| 71 | E53SaCas9KKH2 | CTTTGATACTAACCTTGGT | 86 | E53SaCas9KKH17 | ATACAGTAGATGCAATCCA |
| | | TTC | | | AAA |
| 71 | E53SaCas9KKH2 | CTTTGATACTAACCTTGGT | 87 | E53SaCas9KKH18 | AGGAGGGTCCCTATACAGT |
| | | TTC | | | AGA |
| 71 | E53SaCas9KKH2 | CTTTGATACTAACCTTGGT | 88 | E53SaCas9KKH19 | ATGGAAGGAGGGTCCCTAT |
| | | TTC | | | ACA |
| 71 | E53SaCas9KKH2 | CTTTGATACTAACCTTGGT | 89 | E53SaCas9KKH20 | AGTCATGGAAGGAGGGTCC |
| | | TTC | | | CTA |
| 71 | E53SaCas9KKH2 | CTTTGATACTAACCTTGGT | 90 | E53SaCas9KKH21 | AGCCAAGCTTGAGTCATGG |
| | | TTC | | | AAG |
| 71 | E53SaCas9KKH2 | CTTTGATACTAACCTTGGT | 91 | E53SaCas9KKH22 | TTAGGACAGGCCAGAGCCA |
| | | TTC | | | AGC |
| 71 | E53SaCas9KKH2 | CTTTGATACTAACCTTGGT | 92 | E53SaCas9KKH23 | TGGAAGCTAAGGAAGAAGC |
| | | TTC | | | TGA |
| 71 | E53SaCas9KKH2 | CTTTGATACTAACCTTGGT | 93 | E53SaCas9KKH24 | AATGAAATGTTAAAGGATT |
| | | TTC | | | CAA |
| 71 | E53SaCas9KKH2 | CTTTGATACTAACCTTGGT | 94 | E53SaCas9KKH25 | GCAACAGTTGAATGAAATG |
| | | TTC | | | TTA |
| 71 | E53SaCas9KKH2 | CTTTGATACTAACCTTGGT | 95 | E53SaCas9KKH26 | AGAACCGGAGGCAACAGTT |
| | | TTC | | | GAA |
| 71 | E53SaCas9KKH2 | CTTTGATACTAACCTTGGT | 96 | E53SaCas9KKH27 | CCTTCAGAACCGGAGGCAA |
| | | TTC | | | CAG |
| 71 | E53SaCas9KKH2 | CTTTGATACTAACCTTGGT | 97 | E53SaCas9KKH28 | GAACACCTTCAGAACCGGA |
| | | TTC | | | GGC |
| 71 | E53SaCas9KKH2 | CTTTGATACTAACCTTGGT | 98 | E53SaCas9KKH29 | AAAGAATTCAGAATCAGTG |
| | | TTC | | | GGA |
| 71 | E53SaCas9KKH2 | CTTTGATACTAACCTTGGT | 99 | E53SaCas9KKH30 | AGTTGAAAGAATTCAGAAT |
| | | TTC | | | CAG |
| 71 | E53SaCas9KKH2 | CTTTGATACTAACCTTGGT | 100 | E53SaCas9KKH31 | ATTCTAGTTGAAAGAATTC |
| | | TTC | | | AGA |
| 71 | E53SaCas9KKH2 | CTTTGATACTAACCTTGGT | 101 | E53SaCas9KKH32 | TTTTATTCTAGTTGAAAGA |
| | | TTC | | | ATT |
| 71 | E53SaCas9KKH2 | CTTTGATACTAACCTTGGT | 102 | E53SaCas9KKH33 | TTTTTCCTTTTATTCTAGT |
| | | TTC | | | TGA |
| 72 | E53SaCas9KKH3 | CCTTGGTTTCTGTGATTTT | 73 | E53SaCas9KKH4 | TCCTTAGCTTCCAGCCATT |
| | | CTT | | | GTG |
| 72 | E53SaCas9KKH3 | CCTTGGTTTCTGTGATTTT | 74 | E53SaCas9KKH5 | AACATTTCATTCAACTGTT |
| | | CTT | | | GCC |
| 72 | E53SaCas9KKH3 | CCTTGGTTTCTGTGATTTT | 75 | E53SaCas9KKH6 | TTCAACTGTTGCCTCCGGT |
| | | CTT | | | TCT |
| 72 | E53SaCas9KKH3 | CCTTGGTTTCTGTGATTTT | 76 | E53SaCas9KKH7 | AGGTGTTCTTGTACTTCAT |
| | | CTT | | | CCC |
| 72 | E53SaCas9KKH3 | CCTTGGTTTCTGTGATTTT | 77 | E53SaCas9KKH8 | CTTGTACTTCATCCCACTG |
| | | CTT | | | ATT |
| 72 | E53SaCas9KKH3 | CCTTGGTTTCTGTGATTTT | 78 | E53SaCas9KKH9 | ACTGATTCTGAATTCTTTC |
| | | CTT | | | AAC |
| 72 | E53SaCas9KKH3 | CCTTGGTTTCTGTGATTTT | 84 | E53SaCas9KKH15 | AAAGAAAATCACAGAAACC |
| | | CTT | | | AAG |
| 72 | E53SaCas9KKH3 | CCTTGGTTTCTGTGATTTT | 85 | E53SaCas9KKH16 | TCCAAAAGAAAATCACAGA |
| | | CTT | | | AAC |
| 72 | E53SaCas9KKH3 | CCTTGGTTTCTGTGATTTT | 86 | E53SaCas9KKH17 | ATACAGTAGATGCAATCCA |
| | | CTT | | | AAA |
| 72 | E53SaCas9KKH3 | CCTTGGTTTCTGTGATTTT | 87 | E53SaCas9KKH18 | AGGAGGGTCCCTATACAGT |
| | | CTT | | | AGA |
| 72 | E53SaCas9KKH3 | CCTTGGTTTCTGTGATTTT | 88 | E53SaCas9KKH19 | ATGGAAGGAGGGTCCCTAT |
| | | CTT | | | ACA |
| 72 | E53SaCas9KKH3 | CCTTGGTTTCTGTGATTTT | 89 | E53SaCas9KKH20 | AGTCATGGAAGGAGGGTCC |
| | | CTT | | | CTA |
| 72 | E53SaCas9KKH3 | CCTTGGTTTCTGTGATTTT | 90 | E53SaCas9KKH21 | AGCCAAGCTTGAGTCATGG |
| | | CTT | | | AAG |
| 72 | E53SaCas9KKH3 | CCTTGGTTTCTGTGATTTT | 91 | E53SaCas9KKH22 | TTAGGACAGGCCAGAGCCA |
| | | CTT | | | AGC |
| 72 | E53SaCas9KKH3 | CCTTGGTTTCTGTGATTTT | 92 | E53SaCas9KKH23 | TGGAAGCTAAGGAAGAAGC |
| | | CTT | | | TGA |
| 72 | E53SaCas9KKH3 | CCTTGGTTTCTGTGATTTT | 93 | E53SaCas9KKH24 | AATGAAATGTTAAAGGATT |
| | | CTT | | | CAA |
| 72 | E53SaCas9KKH3 | CCTTGGTTTCTGTGATTTT | 94 | E53SaCas9KKH25 | GCAACAGTTGAATGAAATG |
| | | CTT | | | TTA |
| 72 | E53SaCas9KKH3 | CCTTGGTTTCTGTGATTTT | 95 | E53SaCas9KKH26 | AGAACCGGAGGCAACAGTT |
| | | CTT | | | GAA |
| 72 | E53SaCas9KKH3 | CCTTGGTTTCTGTGATTTT | 96 | E53SaCas9KKH27 | CCTTCAGAACCGGAGGCAA |
| | | CTT | | | CAG |
| 72 | E53SaCas9KKH3 | CCTTGGTTTCTGTGATTTT | 97 | E53SaCas9KKH28 | GAACACCTTCAGAACCGGA |
| | | CTT | | | GGC |
| 72 | E53SaCas9KKH3 | CCTTGGTTTCTGTGATTTT | 98 | E53SaCas9KKH29 | AAAGAATTCAGAATCAGTG |
| | | CTT | | | GGA |
| 72 | E53SaCas9KKH3 | CCTTGGTTTCTGTGATTTT | 99 | E53SaCas9KKH30 | AGTTGAAAGAATTCAGAAT |
| | | CTT | | | CAG |
| 72 | E53SaCas9KKH3 | CCTTGGTTTCTGTGATTTT | 100 | E53SaCas9KKH31 | ATTCTAGTTGAAAGAATTC |
| | | CTT | | | AGA |
| 72 | E53SaCas9KKH3 | CCTTGGTTTCTGTGATTTT | 101 | E53SaCas9KKH32 | TTTTATTCTAGTTGAAAGA |
| | | CTT | | | ATT |
| 72 | E53SaCas9KKH3 | CCTTGGTTTCTGTGATTTT | 102 | E53SaCas9KKH33 | TTTTTCCTTTTATTCTAGT |
| | | CTT | | | TGA |
| 73 | E53SaCas9KKH4 | TCCTTAGCTTCCAGCCATT | 74 | E53SaCas9KKH5 | AACATTTCATTCAACTGTT |
| | | GTG | | | GCC |
| 73 | E53SaCas9KKH4 | TCCTTAGCTTCCAGCCATT | 75 | E53SaCas9KKH6 | TTCAACTGTTGCCTCCGGT |
| | | GTG | | | TCT |
| 73 | E53SaCas9KKH4 | TCCTTAGCTTCCAGCCATT | 76 | E53SaCas9KKH7 | AGGTGTTCTTGTACTTCAT |
| | | GTG | | | CCC |
| 73 | E53SaCas9KKH4 | TCCTTAGCTTCCAGCCATT | 77 | E53SaCas9KKH8 | CTTGTACTTCATCCCACTG |
| | | GTG | | | ATT |
| 73 | E53SaCas9KKH4 | TCCTTAGCTTCCAGCCATT | 78 | E53SaCas9KKH9 | ACTGATTCTGAATTCTTTC |
| | | GTG | | | AAC |
| 73 | E53SaCas9KKH4 | TCCTTAGCTTCCAGCCATT | 84 | E53SaCas9KKH15 | AAAGAAAATCACAGAAACC |
| | | GTG | | | AAG |
| 73 | E53SaCas9KKH4 | TCCTTAGCTTCCAGCCATT | 85 | E53SaCas9KKH16 | TCCAAAAGAAAATCACAGA |
| | | GTG | | | AAC |
| 73 | E53SaCas9KKH4 | TCCTTAGCTTCCAGCCATT | 86 | E53SaCas9KKH17 | ATACAGTAGATGCAATCCA |
| | | GTG | | | AAA |
| 73 | E53SaCas9KKH4 | TCCTTAGCTTCCAGCCATT | 87 | E53SaCas9KKH18 | AGGAGGGTCCCTATACAGT |
| | | GTG | | | AGA |
| 73 | E53SaCas9KKH4 | TCCTTAGCTTCCAGCCATT | 88 | E53SaCas9KKH19 | ATGGAAGGAGGGTCCCTAT |
| | | GTG | | | ACA |
| 73 | E53SaCas9KKH4 | TCCTTAGCTTCCAGCCATT | 89 | E53SaCas9KKH20 | AGTCATGGAAGGAGGGTCC |
| | | GTG | | | CTA |
| 73 | E53SaCas9KKH4 | TCCTTAGCTTCCAGCCATT | 90 | E53SaCas9KKH21 | AGCCAAGCTTGAGTCATGG |
| | | GTG | | | AAG |
| 73 | E53SaCas9KKH4 | TCCTTAGCTTCCAGCCATT | 91 | E53SaCas9KKH22 | TTAGGACAGGCCAGAGCCA |
| | | GTG | | | AGC |
| 73 | E53SaCas9KKH4 | TCCTTAGCTTCCAGCCATT | 92 | E53SaCas9KKH23 | TGGAAGCTAAGGAAGAAGC |
| | | GTG | | | TGA |
| 73 | E53SaCas9KKH4 | TCCTTAGCTTCCAGCCATT | 93 | E53SaCas9KKH24 | AATGAAATGTTAAAGGATT |
| | | GTG | | | CAA |
| 73 | E53SaCas9KKH4 | TCCTTAGCTTCCAGCCATT | 94 | E53SaCas9KKH25 | GCAACAGTTGAATGAAATG |
| | | GTG | | | TTA |
| 73 | E53SaCas9KKH4 | TCCTTAGCTTCCAGCCATT | 95 | E53SaCas9KKH26 | AGAACCGGAGGCAACAGTT |
| | | GTG | | | GAA |
| 73 | E53SaCas9KKH4 | TCCTTAGCTTCCAGCCATT | 96 | E53SaCas9KKH27 | CCTTCAGAACCGGAGGCAA |
| | | GTG | | | CAG |
| 73 | E53SaCas9KKH4 | TCCTTAGCTTCCAGCCATT | 97 | E53SaCas9KKH28 | GAACACCTTCAGAACCGGA |
| | | GTG | | | GGC |
| 73 | E53SaCas9KKH4 | TCCTTAGCTTCCAGCCATT | 98 | E53SaCas9KKH29 | AAAGAATTCAGAATCAGTG |
| | | GTG | | | GGA |
| 73 | E53SaCas9KKH4 | TCCTTAGCTTCCAGCCATT | 99 | E53SaCas9KKH30 | AGTTGAAAGAATTCAGAAT |
| | | GTG | | | CAG |
| 73 | E53SaCas9KKH4 | TCCTTAGCTTCCAGCCATT | 100 | E53SaCas9KKH31 | ATTCTAGTTGAAAGAATTC |
| | | GTG | | | AGA |
| 73 | E53SaCas9KKH4 | TCCTTAGCTTCCAGCCATT | 101 | E53SaCas9KKH32 | TTTTATTCTAGTTGAAAGA |
| | | GTG | | | ATT |
| 73 | E53SaCas9KKH4 | TCCTTAGCTTCCAGCCATT | 102 | E53SaCas9KKH33 | TTTTTCCTTTTATTCTAGT |
| | | GTG | | | TGA |
| 74 | E53SaCas9KKH5 | AACATTTCATTCAACTGTT | 75 | E53SaCas9KKH6 | TTCAACTGTTGCCTCCGGT |
| | | GCC | | | TCT |
| 74 | E53SaCas9KKH5 | AACATTTCATTCAACTGTT | 76 | E53SaCas9KKH7 | AGGTGTTCTTGTACTTCAT |
| | | GCC | | | CCC |
| 74 | E53SaCas9KKH5 | AACATTTCATTCAACTGTT | 77 | E53SaCas9KKH8 | CTTGTACTTCATCCCACTG |
| | | GCC | | | ATT |
| 74 | E53SaCas9KKH5 | AACATTTCATTCAACTGTT | 78 | E53SaCas9KKH9 | ACTGATTCTGAATTCTTTC |
| | | GCC | | | AAC |
| 74 | E53SaCas9KKH5 | AACATTTCATTCAACTGTT | 84 | E53SaCas9KKH15 | AAAGAAAATCACAGAAACC |
| | | GCC | | | AAG |
| 74 | E53SaCas9KKH5 | AACATTTCATTCAACTGTT | 85 | E53SaCas9KKH16 | TCCAAAAGAAAATCACAGA |
| | | GCC | | | AAC |
| 74 | E53SaCas9KKH5 | AACATTTCATTCAACTGTT | 86 | E53SaCas9KKH17 | ATACAGTAGATGCAATCCA |
| | | GCC | | | AAA |
| 74 | E53SaCas9KKH5 | AACATTTCATTCAACTGTT | 87 | E53SaCas9KKH18 | AGGAGGGTCCCTATACAGT |
| | | GCC | | | AGA |
| 74 | E53SaCas9KKH5 | AACATTTCATTCAACTGTT | 88 | E53SaCas9KKH19 | ATGGAAGGAGGGTCCCTAT |
| | | GCC | | | ACA |
| 74 | E53SaCas9KKH5 | AACATTTCATTCAACTGTT | 89 | E53SaCas9KKH20 | AGTCATGGAAGGAGGGTCC |
| | | GCC | | | CTA |
| 74 | E53SaCas9KKH5 | AACATTTCATTCAACTGTT | 90 | E53SaCas9KKH21 | AGCCAAGCTTGAGTCATGG |
| | | GCC | | | AAG |
| 74 | E53SaCas9KKH5 | AACATTTCATTCAACTGTT | 91 | E53SaCas9KKH22 | TTAGGACAGGCCAGAGCCA |
| | | GCC | | | AGC |
| 74 | E53SaCas9KKH5 | AACATTTCATTCAACTGTT | 92 | E53SaCas9KKH23 | TGGAAGCTAAGGAAGAAGC |
| | | GCC | | | TGA |
| 74 | E53SaCas9KKH5 | AACATTTCATTCAACTGTT | 93 | E53SaCas9KKH24 | AATGAAATGTTAAAGGATT |
| | | GCC | | | CAA |
| 74 | E53SaCas9KKH5 | AACATTTCATTCAACTGTT | 94 | E53SaCas9KKH25 | GCAACAGTTGAATGAAATG |
| | | GCC | | | TTA |
| 74 | E53SaCas9KKH5 | AACATTTCATTCAACTGTT | 95 | E53SaCas9KKH26 | AGAACCGGAGGCAACAGTT |
| | | GCC | | | GAA |
| 74 | E53SaCas9KKH5 | AACATTTCATTCAACTGTT | 96 | E53SaCas9KKH27 | CCTTCAGAACCGGAGGCAA |
| | | GCC | | | CAG |
| 74 | E53SaCas9KKH5 | AACATTTCATTCAACTGTT | 97 | E53SaCas9KKH28 | GAACACCTTCAGAACCGGA |
| | | GCC | | | GGC |
| 74 | E53SaCas9KKH5 | AACATTTCATTCAACTGTT | 98 | E53SaCas9KKH29 | AAAGAATTCAGAATCAGTG |
| | | GCC | | | GGA |
| 74 | E53SaCas9KKH5 | AACATTTCATTCAACTGTT | 99 | E53SaCas9KKH30 | AGTTGAAAGAATTCAGAAT |
| | | GCC | | | CAG |
| 74 | E53SaCas9KKH5 | AACATTTCATTCAACTGTT | 100 | E53SaCas9KKH31 | ATTCTAGTTGAAAGAATTC |
| | | GCC | | | AGA |
| 74 | E53SaCas9KKH5 | AACATTTCATTCAACTGTT | 101 | E53SaCas9KKH32 | TTTTATTCTAGTTGAAAGA |
| | | GCC | | | ATT |
| 74 | E53SaCas9KKH5 | AACATTTCATTCAACTGTT | 102 | E53SaCas9KKH33 | TTTTTCCTTTTATTCTAGT |
| | | GCC | | | TGA |
| 75 | E53SaCas9KKH6 | TTCAACTGTTGCCTCCGGT | 76 | E53SaCas9KKH7 | AGGTGTTCTTGTACTTCAT |
| | | TCT | | | CCC |
| 75 | E53SaCas9KKH6 | TTCAACTGTTGCCTCCGGT | 77 | E53SaCas9KKH8 | CTTGTACTTCATCCCACTG |
| | | TCT | | | ATT |
| 75 | E53SaCas9KKH6 | TTCAACTGTTGCCTCCGGT | 78 | E53SaCas9KKH9 | ACTGATTCTGAATTCTTTC |
| | | TCT | | | AAC |
| 75 | E53SaCas9KKH6 | TTCAACTGTTGCCTCCGGT | 84 | E53SaCas9KKH15 | AAAGAAAATCACAGAAACC |
| | | TCT | | | AAG |
| 75 | E53SaCas9KKH6 | TTCAACTGTTGCCTCCGGT | 85 | E53SaCas9KKH16 | TCCAAAAGAAAATCACAGA |
| | | TCT | | | AAC |
| 75 | E53SaCas9KKH6 | TTCAACTGTTGCCTCCGGT | 86 | E53SaCas9KKH17 | ATACAGTAGATGCAATCCA |
| | | TCT | | | AAA |
| 75 | E53SaCas9KKH6 | TTCAACTGTTGCCTCCGGT | 87 | E53SaCas9KKH18 | AGGAGGGTCCCTATACAGT |
| | | TCT | | | AGA |
| 75 | E53SaCas9KKH6 | TTCAACTGTTGCCTCCGGT | 88 | E53SaCas9KKH19 | ATGGAAGGAGGGTCCCTAT |
| | | TCT | | | ACA |
| 75 | E53SaCas9KKH6 | TTCAACTGTTGCCTCCGGT | 89 | E53SaCas9KKH20 | AGTCATGGAAGGAGGGTCC |
| | | TCT | | | CTA |
| 75 | E53SaCas9KKH6 | TTCAACTGTTGCCTCCGGT | 90 | E53SaCas9KKH21 | AGCCAAGCTTGAGTCATGG |
| | | TCT | | | AAG |
| 75 | E53SaCas9KKH6 | TTCAACTGTTGCCTCCGGT | 91 | E53SaCas9KKH22 | TTAGGACAGGCCAGAGCCA |
| | | TCT | | | AGC |
| 75 | E53SaCas9KKH6 | TTCAACTGTTGCCTCCGGT | 92 | E53SaCas9KKH23 | TGGAAGCTAAGGAAGAAGC |
| | | TCT | | | TGA |
| 75 | E53SaCas9KKH6 | TTCAACTGTTGCCTCCGGT | 93 | E53SaCas9KKH24 | AATGAAATGTTAAAGGATT |
| | | TCT | | | CAA |
| 75 | E53SaCas9KKH6 | TTCAACTGTTGCCTCCGGT | 94 | E53SaCas9KKH25 | GCAACAGTTGAATGAAATG |
| | | TCT | | | TTA |
| 75 | E53SaCas9KKH6 | TTCAACTGTTGCCTCCGGT | 95 | E53SaCas9KKH26 | AGAACCGGAGGCAACAGTT |
| | | TCT | | | GAA |
| 75 | E53SaCas9KKH6 | TTCAACTGTTGCCTCCGGT | 96 | E53SaCas9KKH27 | CCTTCAGAACCGGAGGCAA |
| | | TCT | | | CAG |
| 75 | E53SaCas9KKH6 | TTCAACTGTTGCCTCCGGT | 97 | E53SaCas9KKH28 | GAACACCTTCAGAACCGGA |
| | | TCT | | | GGC |
| 75 | E53SaCas9KKH6 | TTCAACTGTTGCCTCCGGT | 98 | E53SaCas9KKH29 | AAAGAATTCAGAATCAGTG |
| | | TCT | | | GGA |
| 75 | E53SaCas9KKH6 | TTCAACTGTTGCCTCCGGT | 99 | E53SaCas9KKH30 | AGTTGAAAGAATTCAGAAT |
| | | TCT | | | CAG |
| 75 | E53SaCas9KKH6 | TTCAACTGTTGCCTCCGGT | 100 | E53SaCas9KKH31 | ATTCTAGTTGAAAGAATTC |
| | | TCT | | | AGA |
| 75 | E53SaCas9KKH6 | TTCAACTGTTGCCTCCGGT | 101 | E53SaCas9KKH32 | TTTTATTCTAGTTGAAAGA |
| | | TCT | | | ATT |
| 75 | E53SaCas9KKH6 | TTCAACTGTTGCCTCCGGT | 102 | E53SaCas9KKH33 | TTTTTCCTTTTATTCTAGT |
| | | TCT | | | TGA |
| 76 | E53SaCas9KKH7 | AGGTGTTCTTGTACTTCAT | 77 | E53SaCas9KKH8 | CTTGTACTTCATCCCACTG |
| | | CCC | | | ATT |
| 76 | E53SaCas9KKH7 | AGGTGTTCTTGTACTTCAT | 78 | E53SaCas9KKH9 | ACTGATTCTGAATTCTTTC |
| | | CCC | | | AAC |
| 76 | E53SaCas9KKH7 | AGGTGTTCTTGTACTTCAT | 84 | E53SaCas9KKH15 | AAAGAAAATCACAGAAACC |
| | | CCC | | | AAG |
| 76 | E53SaCas9KKH7 | AGGTGTTCTTGTACTTCAT | 85 | E53SaCas9KKH16 | TCCAAAAGAAAATCACAGA |
| | | CCC | | | AAC |
| 76 | E53SaCas9KKH7 | AGGTGTTCTTGTACTTCAT | 86 | E53SaCas9KKH17 | ATACAGTAGATGCAATCCA |
| | | CCC | | | AAA |
| 76 | E53SaCas9KKH7 | AGGTGTTCTTGTACTTCAT | 87 | E53SaCas9KKH18 | AGGAGGGTCCCTATACAGT |
| | | CCC | | | AGA |
| 76 | E53SaCas9KKH7 | AGGTGTTCTTGTACTTCAT | 88 | E53SaCas9KKH19 | ATGGAAGGAGGGTCCCTAT |
| | | CCC | | | ACA |
| 76 | E53SaCas9KKH7 | AGGTGTTCTTGTACTTCAT | 89 | E53SaCas9KKH20 | AGTCATGGAAGGAGGGTCC |
| | | CCC | | | CTA |
| 76 | E53SaCas9KKH7 | AGGTGTTCTTGTACTTCAT | 90 | E53SaCas9KKH21 | AGCCAAGCTTGAGTCATGG |
| | | CCC | | | AAG |
| 76 | E53SaCas9KKH7 | AGGTGTTCTTGTACTTCAT | 91 | E53SaCas9KKH22 | TTAGGACAGGCCAGAGCCA |
| | | CCC | | | AGC |
| 76 | E53SaCas9KKH7 | AGGTGTTCTTGTACTTCAT | 92 | E53SaCas9KKH23 | TGGAAGCTAAGGAAGAAGC |
| | | CCC | | | TGA |
| 76 | E53SaCas9KKH7 | AGGTGTTCTTGTACTTCAT | 93 | E53SaCas9KKH24 | AATGAAATGTTAAAGGATT |
| | | CCC | | | CAA |
| 76 | E53SaCas9KKH7 | AGGTGTTCTTGTACTTCAT | 94 | E53SaCas9KKH25 | GCAACAGTTGAATGAAATG |
| | | CCC | | | TTA |
| 76 | E53SaCas9KKH7 | AGGTGTTCTTGTACTTCAT | 95 | E53SaCas9KKH26 | AGAACCGGAGGCAACAGTT |
| | | CCC | | | GAA |
| 76 | E53SaCas9KKH7 | AGGTGTTCTTGTACTTCAT | 96 | E53SaCas9KKH27 | CCTTCAGAACCGGAGGCAA |
| | | CCC | | | CAG |
| 76 | E53SaCas9KKH7 | AGGTGTTCTTGTACTTCAT | 97 | E53SaCas9KKH28 | GAACACCTTCAGAACCGGA |
| | | CCC | | | GGC |
| 76 | E53SaCas9KKH7 | AGGTGTTCTTGTACTTCAT | 98 | E53SaCas9KKH29 | AAAGAATTCAGAATCAGTG |
| | | CCC | | | GGA |
| 76 | E53SaCas9KKH7 | AGGTGTTCTTGTACTTCAT | 99 | E53SaCas9KKH30 | AGTTGAAAGAATTCAGAAT |
| | | CCC | | | CAG |
| 76 | E53SaCas9KKH7 | AGGTGTTCTTGTACTTCAT | 100 | E53SaCas9KKH31 | ATTCTAGTTGAAAGAATTC |
| | | CCC | | | AGA |
| 76 | E53SaCas9KKH7 | AGGTGTTCTTGTACTTCAT | 101 | E53SaCas9KKH32 | TTTTATTCTAGTTGAAAGA |
| | | CCC | | | ATT |
| 76 | E53SaCas9KKH7 | AGGTGTTCTTGTACTTCAT | 102 | E53SaCas9KKH33 | TTTTTCCTTTTATTCTAGT |
| | | CCC | | | TGA |
| 77 | E53SaCas9KKH8 | CTTGTACTTCATCCCACTG | 78 | E53SaCas9KKH9 | ACTGATTCTGAATTCTTTC |
| | | ATT | | | AAC |
| 77 | E53SaCas9KKH8 | CTTGTACTTCATCCCACTG | 84 | E53SaCas9KKH15 | AAAGAAAATCACAGAAACC |
| | | ATT | | | AAG |
| 77 | E53SaCas9KKH8 | CTTGTACTTCATCCCACTG | 85 | E53SaCas9KKH16 | TCCAAAAGAAAATCACAGA |
| | | ATT | | | AAC |
| 77 | E53SaCas9KKH8 | CTTGTACTTCATCCCACTG | 86 | E53SaCas9KKH17 | ATACAGTAGATGCAATCCA |
| | | ATT | | | AAA |
| 77 | E53SaCas9KKH8 | CTTGTACTTCATCCCACTG | 87 | E53SaCas9KKH18 | AGGAGGGTCCCTATACAGT |
| | | ATT | | | AGA |
| 77 | E53SaCas9KKH8 | CTTGTACTTCATCCCACTG | 88 | E53SaCas9KKH19 | ATGGAAGGAGGGTCCCTAT |
| | | ATT | | | ACA |
| 77 | E53SaCas9KKH8 | CTTGTACTTCATCCCACTG | 89 | E53SaCas9KKH20 | AGTCATGGAAGGAGGGTCC |
| | | ATT | | | CTA |
| 77 | E53SaCas9KKH8 | CTTGTACTTCATCCCACTG | 90 | E53SaCas9KKH21 | AGCCAAGCTTGAGTCATGG |
| | | ATT | | | AAG |
| 77 | E53SaCas9KKH8 | CTTGTACTTCATCCCACTG | 91 | E53SaCas9KKH22 | TTAGGACAGGCCAGAGCCA |
| | | ATT | | | AGC |
| 77 | E53SaCas9KKH8 | CTTGTACTTCATCCCACTG | 92 | E53SaCas9KKH23 | TGGAAGCTAAGGAAGAAGC |
| | | ATT | | | TGA |
| 77 | E53SaCas9KKH8 | CTTGTACTTCATCCCACTG | 93 | E53SaCas9KKH24 | AATGAAATGTTAAAGGATT |
| | | ATT | | | CAA |
| 77 | E53SaCas9KKH8 | CTTGTACTTCATCCCACTG | 94 | E53SaCas9KKH25 | GCAACAGTTGAATGAAATG |
| | | ATT | | | TTA |
| 77 | E53SaCas9KKH8 | CTTGTACTTCATCCCACTG | 95 | E53SaCas9KKH26 | AGAACCGGAGGCAACAGTT |
| | | ATT | | | GAA |
| 77 | E53SaCas9KKH8 | CTTGTACTTCATCCCACTG | 96 | E53SaCas9KKH27 | CCTTCAGAACCGGAGGCAA |
| | | ATT | | | CAG |
| 77 | E53SaCas9KKH8 | CTTGTACTTCATCCCACTG | 97 | E53SaCas9KKH28 | GAACACCTTCAGAACCGGA |
| | | ATT | | | GGC |
| 77 | E53SaCas9KKH8 | CTTGTACTTCATCCCACTG | 98 | E53SaCas9KKH29 | AAAGAATTCAGAATCAGTG |
| | | ATT | | | GGA |
| 77 | E53SaCas9KKH8 | CTTGTACTTCATCCCACTG | 99 | E53SaCas9KKH30 | AGTTGAAAGAATTCAGAAT |
| | | ATT | | | CAG |
| 77 | E53SaCas9KKH8 | CTTGTACTTCATCCCACTG | 100 | E53SaCas9KKH31 | ATTCTAGTTGAAAGAATTC |
| | | ATT | | | AGA |
| 77 | E53SaCas9KKH8 | CTTGTACTTCATCCCACTG | 101 | E53SaCas9KKH32 | TTTTATTCTAGTTGAAAGA |
| | | ATT | | | ATT |
| 77 | E53SaCas9KKH8 | CTTGTACTTCATCCCACTG | 102 | E53SaCas9KKH33 | TTTTTCCTTTTATTCTAGT |
| | | ATT | | | TGA |
| 78 | E53SaCas9KKH9 | ACTGATTCTGAATTCTTTC | 84 | E53SaCas9KKH15 | AAAGAAAATCACAGAAACC |
| | | AAC | | | AAG |
| 78 | E53SaCas9KKH9 | ACTGATTCTGAATTCTTTC | 85 | E53SaCas9KKH16 | TCCAAAAGAAAATCACAGA |
| | | AAC | | | AAC |
| 78 | E53SaCas9KKH9 | ACTGATTCTGAATTCTTTC | 86 | E53SaCas9KKH17 | ATACAGTAGATGCAATCCA |
| | | AAC | | | AAA |
| 78 | E53SaCas9KKH9 | ACTGATTCTGAATTCTTTC | 87 | E53SaCas9KKH18 | AGGAGGGTCCCTATACAGT |
| | | AAC | | | AGA |
| 78 | E53SaCas9KKH9 | ACTGATTCTGAATTCTTTC | 88 | E53SaCas9KKH19 | ATGGAAGGAGGGTCCCTAT |
| | | AAC | | | ACA |
| 78 | E53SaCas9KKH9 | ACTGATTCTGAATTCTTTC | 89 | E53SaCas9KKH20 | AGTCATGGAAGGAGGGTCC |
| | | AAC | | | CTA |
| 78 | E53SaCas9KKH9 | ACTGATTCTGAATTCTTTC | 90 | E53SaCas9KKH21 | AGCCAAGCTTGAGTCATGG |
| | | AAC | | | AAG |
| 78 | E53SaCas9KKH9 | ACTGATTCTGAATTCTTTC | 91 | E53SaCas9KKH22 | TTAGGACAGGCCAGAGCCA |
| | | AAC | | | AGC |
| 78 | E53SaCas9KKH9 | ACTGATTCTGAATTCTTTC | 92 | E53SaCas9KKH23 | TGGAAGCTAAGGAAGAAGC |
| | | AAC | | | TGA |
| 78 | E53SaCas9KKH9 | ACTGATTCTGAATTCTTTC | 93 | E53SaCas9KKH24 | AATGAAATGTTAAAGGATT |
| | | AAC | | | CAA |
| 78 | E53SaCas9KKH9 | ACTGATTCTGAATTCTTTC | 94 | E53SaCas9KKH25 | GCAACAGTTGAATGAAATG |
| | | AAC | | | TTA |
| 78 | E53SaCas9KKH9 | ACTGATTCTGAATTCTTTC | 95 | E53SaCas9KKH26 | AGAACCGGAGGCAACAGTT |
| | | AAC | | | GAA |
| 78 | E53SaCas9KKH9 | ACTGATTCTGAATTCTTTC | 96 | E53SaCas9KKH27 | CCTTCAGAACCGGAGGCAA |
| | | AAC | | | CAG |
| 78 | E53SaCas9KKH9 | ACTGATTCTGAATTCTTTC | 97 | E53SaCas9KKH | GAACACCTTCAGAACCGGA |
| | | AAC | | 28 | GGC |
| 78 | E53SaCas9KKH9 | ACTGATTCTGAATTCTTTC | 98 | E53SaCas9KKH | AAAGAATTCAGAATCAGTG |
| | | AAC | | 29 | GGA |
| 78 | E53SaCas9KKH9 | ACTGATTCTGAATTCTTTC | 99 | E53SaCas9KKH | AGTTGAAAGAATTCAGAAT |
| | | AAC | | 30 | CAG |
| 78 | E53SaCas9KKH9 | ACTGATTCTGAATTCTTTC | 100 | E53SaCas9KKH | ATTCTAGTTGAAAGAATTC |
| | | AAC | | 31 | AGA |
| 78 | E53SaCas9KKH9 | ACTGATTCTGAATTCTTTC | 101 | E53SaCas9KKH | TTTTATTCTAGTTGAAAGA |
| | | AAC | | 32 | ATT |
| 78 | E53SaCas9KKH9 | ACTGATTCTGAATTCTTTC | 102 | E53SaCas9KKH | TTTTTCCTTTTATTCTAGT |
| | | AAC | | 33 | TGA |
| 84 | E53SaCas9KKH | AAAGAAAATCACAGAAACC | 85 | E53SaCas9KKH | TCCAAAAGAAAATCACAGA |
| | 15 | AAG | | 16 | AAC |
| 84 | E53SaCas9KKH | AAAGAAAATCACAGAAACC | 86 | E53SaCas9KKH | ATACAGTAGATGCAATCCA |
| | 15 | AAG | | 17 | AAA |
| 84 | E53SaCas9KKH | AAAGAAAATCACAGAAACC | 87 | E53SaCas9KKH | AGGAGGGTCCCTATACAGT |
| | 15 | AAG | | 18 | AGA |
| 84 | E53SaCas9KKH | AAAGAAAATCACAGAAACC | 88 | E53SaCas9KKH | ATGGAAGGAGGGTCCCTAT |
| | 15 | AAG | | 19 | ACA |
| 84 | E53SaCas9KKH | AAAGAAAATCACAGAAACC | 89 | E53SaCas9KKH | AGTCATGGAAGGAGGGTCC |
| | 15 | AAG | | 20 | CTA |
| 84 | E53SaCas9KKH | AAAGAAAATCACAGAAACC | 90 | E53SaCas9KKH | AGCCAAGCTTGAGTCATGG |
| | 15 | AAG | | 21 | AAG |
| 84 | E53SaCas9KKH | AAAGAAAATCACAGAAACC | 91 | E53SaCas9KKH | TTAGGACAGGCCAGAGCCA |
| | 15 | AAG | | 22 | AGC |
| 84 | E53SaCas9KKH | AAAGAAAATCACAGAAACC | 92 | E53SaCas9KKH | TGGAAGCTAAGGAAGAAGC |
| | 15 | AAG | | 23 | TGA |
| 84 | E53SaCas9KKH | AAAGAAAATCACAGAAACC | 93 | E53SaCas9KKH | AATGAAATGTTAAAGGATT |
| | 15 | AAG | | 24 | CAA |
| 84 | E53SaCas9KKH | AAAGAAAATCACAGAAACC | 94 | E53SaCas9KKH | GCAACAGTTGAATGAAATG |
| | 15 | AAG | | 25 | TTA |
| 84 | E53SaCas9KKH | AAAGAAAATCACAGAAACC | 95 | E53SaCas9KKH | AGAACCGGAGGCAACAGTT |
| | 15 | AAG | | 26 | GAA |
| 84 | E53SaCas9KKH | AAAGAAAATCACAGAAACC | 96 | E53SaCas9KKH | CCTTCAGAACCGGAGGCAA |
| | 15 | AAG | | 27 | CAG |
| 84 | E53SaCas9KKH | AAAGAAAATCACAGAAACC | 97 | E53SaCas9KKH | GAACACCTTCAGAACCGGA |
| | 15 | AAG | | 28 | GGC |
| 84 | E53SaCas9KKH | AAAGAAAATCACAGAAACC | 98 | E53SaCas9KKH | AAAGAATTCAGAATCAGTG |
| | 15 | AAG | | 29 | GGA |
| 84 | E53SaCas9KKH | AAAGAAAATCACAGAAACC | 99 | E53SaCas9KKH | AGTTGAAAGAATTCAGAAT |
| | 15 | AAG | | 30 | CAG |
| 84 | E53SaCas9KKH | AAAGAAAATCACAGAAACC | 100 | E53SaCas9KKH | ATTCTAGTTGAAAGAATTC |
| | 15 | AAG | | 31 | AGA |
| 84 | E53SaCas9KKH | AAAGAAAATCACAGAAACC | 101 | E53SaCas9KKH | TTTTATTCTAGTTGAAAGA |
| | 15 | AAG | | 32 | ATT |
| 84 | E53SaCas9KKH | AAAGAAAATCACAGAAACC | 102 | E53SaCas9KKH | TTTTTCCTTTTATTCTAGT |
| | 15 | AAG | | 33 | TGA |
| 85 | E53SaCas9KKH | TCCAAAAGAAAATCACAGA | 86 | E53SaCas9KKH | ATACAGTAGATGCAATCCA |
| | 16 | AAC | | 17 | AAA |
| 85 | E53SaCas9KKH | TCCAAAAGAAAATCACAGA | 87 | E53SaCas9KKH | AGGAGGGTCCCTATACAGT |
| | 16 | AAC | | 18 | AGA |
| 85 | E53SaCas9KKH | TCCAAAAGAAAATCACAGA | 88 | E53SaCas9KKH | ATGGAAGGAGGGTCCCTAT |
| | 16 | AAC | | 19 | ACA |
| 85 | E53SaCas9KKH | TCCAAAAGAAAATCACAGA | 89 | E53SaCas9KKH | AGTCATGGAAGGAGGGTCC |
| | 16 | AAC | | 20 | CTA |
| 85 | E53SaCas9KKH | TCCAAAAGAAAATCACAGA | 90 | E53SaCas9KKH | AGCCAAGCTTGAGTCATGG |
| | 16 | AAC | | 21 | AAG |
| 85 | E53SaCas9KKH | TCCAAAAGAAAATCACAGA | 91 | E53SaCas9KKH | TTAGGACAGGCCAGAGCCA |
| | 16 | AAC | | 22 | AGC |
| 85 | E53SaCas9KKH | TCCAAAAGAAAATCACAGA | 92 | E53SaCas9KKH | TGGAAGCTAAGGAAGAAGC |
| | 16 | AAC | | 23 | TGA |
| 85 | E53SaCas9KKH | TCCAAAAGAAAATCACAGA | 93 | E53SaCas9KKH | AATGAAATGTTAAAGGATT |
| | 16 | AAC | | 24 | CAA |
| 85 | E53SaCas9KKH | TCCAAAAGAAAATCACAGA | 94 | E53SaCas9KKH | GCAACAGTTGAATGAAATG |
| | 16 | AAC | | 25 | TTA |
| 85 | E53SaCas9KKH | TCCAAAAGAAAATCACAGA | 95 | E53SaCas9KKH | AGAACCGGAGGCAACAGTT |
| | 16 | AAC | | 26 | GAA |
| 85 | E53SaCas9KKH | TCCAAAAGAAAATCACAGA | 96 | E53SaCas9KKH | CCTTCAGAACCGGAGGCAA |
| | 16 | AAC | | 27 | CAG |
| 85 | E53SaCas9KKH | TCCAAAAGAAAATCACAGA | 97 | E53SaCas9KKH | GAACACCTTCAGAACCGGA |
| | 16 | AAC | | 28 | GGC |
| 85 | E53SaCas9KKH | TCCAAAAGAAAATCACAGA | 98 | E53SaCas9KKH | AAAGAATTCAGAATCAGTG |
| | 16 | AAC | | 29 | GGA |
| 85 | E53SaCas9KKH | TCCAAAAGAAAATCACAGA | 99 | E53SaCas9KKH | AGTTGAAAGAATTCAGAAT |
| | 16 | AAC | | 30 | CAG |
| 85 | E53SaCas9KKH | TCCAAAAGAAAATCACAGA | 100 | E53SaCas9KKH | ATTCTAGTTGAAAGAATTC |
| | 16 | AAC | | 31 | AGA |
| 85 | E53SaCas9KKH | TCCAAAAGAAAATCACAGA | 101 | E53SaCas9KKH | TTTTATTCTAGTTGAAAGA |
| | 16 | AAC | | 32 | ATT |
| 85 | E53SaCas9KKH | TCCAAAAGAAAATCACAGA | 102 | E53SaCas9KKH | TTTTTCCTTTTATTCTAGT |
| | 16 | AAC | | 33 | TGA |
| 86 | E53SaCas9KKH | ATACAGTAGATGCAATCCA | 87 | E53SaCas9KKH | AGGAGGGTCCCTATACAGT |
| | 17 | AAA | | 18 | AGA |
| 86 | E53SaCas9KKH | ATACAGTAGATGCAATCCA | 88 | E53SaCas9KKH | ATGGAAGGAGGGTCCCTAT |
| | 17 | AAA | | 19 | ACA |
| 86 | E53SaCas9KKH | ATACAGTAGATGCAATCCA | 89 | E53SaCas9KKH | AGTCATGGAAGGAGGGTCC |
| | 17 | AAA | | 20 | CTA |
| 86 | E53SaCas9KKH | ATACAGTAGATGCAATCCA | 90 | E53SaCas9KKH | AGCCAAGCTTGAGTCATGG |
| | 17 | AAA | | 21 | AAG |
| 86 | E53SaCas9KKH | ATACAGTAGATGCAATCCA | 91 | E53SaCas9KKH | TTAGGACAGGCCAGAGCCA |
| | 17 | AAA | | 22 | AGC |
| 86 | E53SaCas9KKH | ATACAGTAGATGCAATCCA | 92 | E53SaCas9KKH | TGGAAGCTAAGGAAGAAGC |
| | 17 | AAA | | 23 | TGA |
| 86 | E53SaCas9KKH | ATACAGTAGATGCAATCCA | 93 | E53SaCas9KKH | AATGAAATGTTAAAGGATT |
| | 17 | AAA | | 24 | CAA |
| 86 | E53SaCas9KKH | ATACAGTAGATGCAATCCA | 94 | E53SaCas9KKH | GCAACAGTTGAATGAAATG |
| | 17 | AAA | | 25 | TTA |
| 86 | E53SaCas9KKH | ATACAGTAGATGCAATCCA | 95 | E53SaCas9KKH | AGAACCGGAGGCAACAGTT |
| | 17 | AAA | | 26 | GAA |
| 86 | E53SaCas9KKH | ATACAGTAGATGCAATCCA | 96 | E53SaCas9KKH | CCTTCAGAACCGGAGGCAA |
| | 17 | AAA | | 27 | CAG |
| 86 | E53SaCas9KKH | ATACAGTAGATGCAATCCA | 97 | E53SaCas9KKH | GAACACCTTCAGAACCGGA |
| | 17 | AAA | | 28 | GGC |
| 86 | E53SaCas9KKH | ATACAGTAGATGCAATCCA | 98 | E53SaCas9KKH | AAAGAATTCAGAATCAGTG |
| | 17 | AAA | | 29 | GGA |
| 86 | E53SaCas9KKH | ATACAGTAGATGCAATCCA | 99 | E53SaCas9KKH | AGTTGAAAGAATTCAGAAT |
| | 17 | AAA | | 30 | CAG |
| 86 | E53SaCas9KKH | ATACAGTAGATGCAATCCA | 100 | E53SaCas9KKH | ATTCTAGTTGAAAGAATTC |
| | 17 | AAA | | 31 | AGA |
| 86 | E53SaCas9KKH | ATACAGTAGATGCAATCCA | 101 | E53SaCas9KKH | TTTTATTCTAGTTGAAAGA |
| | 17 | AAA | | 32 | ATT |
| 86 | E53SaCas9KKH | ATACAGTAGATGCAATCCA | 102 | E53SaCas9KKH | TTTTTCCTTTTATTCTAGT |
| | 17 | AAA | | 33 | TGA |
| 87 | E53SaCas9KKH | AGGAGGGTCCCTATACAGT | 88 | E53SaCas9KKH | ATGGAAGGAGGGTCCCTAT |
| | 18 | AGA | | 19 | ACA |
| 87 | E53SaCas9KKH | AGGAGGGTCCCTATACAGT | 89 | E53SaCas9KKH | AGTCATGGAAGGAGGGTCC |
| | 18 | AGA | | 20 | CTA |
| 87 | E53SaCas9KKH | AGGAGGGTCCCTATACAGT | 90 | E53SaCas9KKH | AGCCAAGCTTGAGTCATGG |
| | 18 | AGA | | 21 | AAG |
| 87 | E53SaCas9KKH | AGGAGGGTCCCTATACAGT | 91 | E53SaCas9KKH | TTAGGACAGGCCAGAGCCA |
| | 18 | AGA | | 22 | AGC |
| 87 | E53SaCas9KKH | AGGAGGGTCCCTATACAGT | 92 | E53SaCas9KKH | TGGAAGCTAAGGAAGAAGC |
| | 18 | AGA | | 23 | TGA |
| 87 | E53SaCas9KKH | AGGAGGGTCCCTATACAGT | 93 | E53SaCas9KKH | AATGAAATGTTAAAGGATT |
| | 18 | AGA | | 24 | CAA |
| 87 | E53SaCas9KKH | AGGAGGGTCCCTATACAGT | 94 | E53SaCas9KKH | GCAACAGTTGAATGAAATG |
| | 18 | AGA | | 25 | TTA |
| 87 | E53SaCas9KKH | AGGAGGGTCCCTATACAGT | 95 | E53SaCas9KKH | AGAACCGGAGGCAACAGTT |
| | 18 | AGA | | 26 | GAA |
| 87 | E53SaCas9KKH | AGGAGGGTCCCTATACAGT | 96 | E53SaCas9KKH | CCTTCAGAACCGGAGGCAA |
| | 18 | AGA | | 27 | CAG |
| 87 | E53SaCas9KKH | AGGAGGGTCCCTATACAGT | 97 | E53SaCas9KKH | GAACACCTTCAGAACCGGA |
| | 18 | AGA | | 28 | GGC |
| 87 | E53SaCas9KKH | AGGAGGGTCCCTATACAGT | 98 | E53SaCas9KKH | AAAGAATTCAGAATCAGTG |
| | 18 | AGA | | 29 | GGA |
| 87 | E53SaCas9KKH | AGGAGGGTCCCTATACAGT | 99 | E53SaCas9KKH | AGTTGAAAGAATTCAGAAT |
| | 18 | AGA | | 30 | CAG |
| 87 | E53SaCas9KKH | AGGAGGGTCCCTATACAGT | 100 | E53SaCas9KKH | ATTCTAGTTGAAAGAATTC |
| | 18 | AGA | | 31 | AGA |
| 87 | E53SaCas9KKH | AGGAGGGTCCCTATACAGT | 101 | E53SaCas9KKH | TTTTATTCTAGTTGAAAGA |
| | 18 | AGA | | 32 | ATT |
| 87 | E53SaCas9KKH | AGGAGGGTCCCTATACAGT | 102 | E53SaCas9KKH | TTTTTCCTTTTATTCTAGT |
| | 18 | AGA | | 33 | TGA |
| 88 | E53SaCas9KKH | ATGGAAGGAGGGTCCCTAT | 89 | E53SaCas9KKH | AGTCATGGAAGGAGGGTCC |
| | 19 | ACA | | 20 | CTA |
| 88 | E53SaCas9KKH | ATGGAAGGAGGGTCCCTAT | 90 | E53SaCas9KKH | AGCCAAGCTTGAGTCATGG |
| | 19 | ACA | | 21 | AAG |
| 88 | E53SaCas9KKH | ATGGAAGGAGGGTCCCTAT | 91 | E53SaCas9KKH | TTAGGACAGGCCAGAGCCA |
| | 19 | ACA | | 22 | AGC |
| 88 | E53SaCas9KKH | ATGGAAGGAGGGTCCCTAT | 92 | E53SaCas9KKH | TGGAAGCTAAGGAAGAAGC |
| | 19 | ACA | | 23 | TGA |
| 88 | E53SaCas9KKH | ATGGAAGGAGGGTCCCTAT | 93 | E53SaCas9KKH | AATGAAATGTTAAAGGATT |
| | 19 | ACA | | 24 | CAA |
| 88 | E53SaCas9KKH | ATGGAAGGAGGGTCCCTAT | 94 | E53SaCas9KKH | GCAACAGTTGAATGAAATG |
| | 19 | ACA | | 25 | TTA |
| 88 | E53SaCas9KKH | ATGGAAGGAGGGTCCCTAT | 95 | E53SaCas9KKH | AGAACCGGAGGCAACAGTT |
| | 19 | ACA | | 26 | GAA |
| 88 | E53SaCas9KKH | ATGGAAGGAGGGTCCCTAT | 96 | E53SaCas9KKH | CCTTCAGAACCGGAGGCAA |
| | 19 | ACA | | 27 | CAG |
| 88 | E53SaCas9KKH | ATGGAAGGAGGGTCCCTAT | 97 | E53SaCas9KKH | GAACACCTTCAGAACCGGA |
| | 19 | ACA | | 28 | GGC |
| 88 | E53SaCas9KKH | ATGGAAGGAGGGTCCCTAT | 98 | E53SaCas9KKH | AAAGAATTCAGAATCAGTG |
| | 19 | ACA | | 29 | GGA |
| 88 | E53SaCas9KKH | ATGGAAGGAGGGTCCCTAT | 99 | E53SaCas9KKH | AGTTGAAAGAATTCAGAAT |
| | 19 | ACA | | 30 | CAG |
| 88 | E53SaCas9KKH | ATGGAAGGAGGGTCCCTAT | 100 | E53SaCas9KKH | ATTCTAGTTGAAAGAATTC |
| | 19 | ACA | | 31 | AGA |
| 88 | E53SaCas9KKH | ATGGAAGGAGGGTCCCTAT | 101 | E53SaCas9KKH | TTTTATTCTAGTTGAAAGA |
| | 19 | ACA | | 32 | ATT |
| 88 | E53SaCas9KKH | ATGGAAGGAGGGTCCCTAT | 102 | E53SaCas9KKH | TTTTTCCTTTTATTCTAGT |
| | 19 | ACA | | 33 | TGA |
| 89 | E53SaCas9KKH | AGTCATGGAAGGAGGGTCC | 90 | E53SaCas9KKH | AGCCAAGCTTGAGTCATGG |
| | 20 | CTA | | 21 | AAG |
| 89 | E53SaCas9KKH | AGTCATGGAAGGAGGGTCC | 91 | E53SaCas9KKH | TTAGGACAGGCCAGAGCCA |
| | 20 | CTA | | 22 | AGC |
| 89 | E53SaCas9KKH | AGTCATGGAAGGAGGGTCC | 92 | E53SaCas9KKH | TGGAAGCTAAGGAAGAAGC |
| | 20 | CTA | | 23 | TGA |
| 89 | E53SaCas9KKH | AGTCATGGAAGGAGGGTCC | 93 | E53SaCas9KKH | AATGAAATGTTAAAGGATT |
| | 20 | CTA | | 24 | CAA |
| 89 | E53SaCas9KKH | AGTCATGGAAGGAGGGTCC | 94 | E53SaCas9KKH | GCAACAGTTGAATGAAATG |
| | 20 | CTA | | 25 | TTA |
| 89 | E53SaCas9KKH | AGTCATGGAAGGAGGGTCC | 95 | E53SaCas9KKH | AGAACCGGAGGCAACAGTT |
| | 20 | CTA | | 26 | GAA |
| 89 | E53SaCas9KKH | AGTCATGGAAGGAGGGTCC | 96 | E53SaCas9KKH | CCTTCAGAACCGGAGGCAA |
| | 20 | CTA | | 27 | CAG |
| 89 | E53SaCas9KKH | AGTCATGGAAGGAGGGTCC | 97 | E53SaCas9KKH | GAACACCTTCAGAACCGGA |
| | 20 | CTA | | 28 | GGC |
| 89 | E53SaCas9KKH | AGTCATGGAAGGAGGGTCC | 98 | E53SaCas9KKH | AAAGAATTCAGAATCAGTG |
| | 20 | CTA | | 29 | GGA |
| 89 | E53SaCas9KKH | AGTCATGGAAGGAGGGTCC | 99 | E53SaCas9KKH | AGTTGAAAGAATTCAGAAT |
| | 20 | CTA | | 30 | CAG |
| 89 | E53SaCas9KKH | AGTCATGGAAGGAGGGTCC | 100 | E53SaCas9KKH | ATTCTAGTTGAAAGAATTC |
| | 20 | CTA | | 31 | AGA |
| 89 | E53SaCas9KKH | AGTCATGGAAGGAGGGTCC | 101 | E53SaCas9KKH | TTTTATTCTAGTTGAAAGA |
| | 20 | CTA | | 32 | ATT |
| 89 | E53SaCas9KKH | AGTCATGGAAGGAGGGTCC | 102 | E53SaCas9KKH | TTTTTCCTTTTATTCTAGT |
| | 20 | CTA | | 33 | TGA |
| 90 | E53SaCas9KKH | AGCCAAGCTTGAGTCATGG | 91 | E53SaCas9KKH | TTAGGACAGGCCAGAGCCA |
| | 21 | AAG | | 22 | AGC |
| 90 | E53SaCas9KKH | AGCCAAGCTTGAGTCATGG | 92 | E53SaCas9KKH | TGGAAGCTAAGGAAGAAGC |
| | 21 | AAG | | 23 | TGA |
| 9C | E53SaCas9KKH | AGCCAAGCTTGAGTCATGG | 93 | E53SaCas9KKH | AATGAAATGTTAAAGGATT |
| | 21 | AAG | | 24 | CAA |
| 90 | E53SaCas9KKH | AGCCAAGCTTGAGTCATGG | 94 | E53SaCas9KKH | GCAACAGTTGAATGAAATG |
| | 21 | AAG | | 25 | TTA |
| 90 | E53SaCas9KKH | AGCCAAGCTTGAGTCATGG | 95 | E53SaCas9KKH | AGAACCGGAGGCAACAGTT |
| | 21 | AAG | | 26 | GAA |
| 90 | E53SaCas9KKH | AGCCAAGCTTGAGTCATGG | 96 | E53SaCas9KKH | CCTTCAGAACCGGAGGCAA |
| | 21 | AAG | | 27 | CAG |
| 90 | E53SaCas9KKH | AGCCAAGCTTGAGTCATGG | 97 | E53SaCas9KKH | GAACACCTTCAGAACCGGA |
| | 21 | AAG | | 28 | GGC |
| 90 | E53SaCas9KKH | AGCCAAGCTTGAGTCATGG | 98 | E53SaCas9KKH | AAAGAATTCAGAATCAGTG |
| | 21 | AAG | | 29 | GGA |
| 90 | E53SaCas9KKH | AGCCAAGCTTGAGTCATGG | 99 | E53SaCas9KKH | AGTTGAAAGAATTCAGAAT |
| | 21 | AAG | | 30 | CAG |
| 90 | E53SaCas9KKH | AGCCAAGCTTGAGTCATGG | 100 | E53SaCas9KKH | ATTCTAGTTGAAAGAATTC |
| | 21 | AAG | | 31 | AGA |
| 90 | E53SaCas9KKH | AGCCAAGCTTGAGTCATGG | 101 | E53SaCas9KKH | TTTTATTCTAGTTGAAAGA |
| | 21 | AAG | | 32 | ATT |
| 90 | E53SaCas9KKH | AGCCAAGCTTGAGTCATGG | 102 | E53SaCas9KKH | TTTTTCCTTTTATTCTAGT |
| | 21 | AAG | | 33 | TGA |
| 91 | E53SaCas9KKH | TTAGGACAGGCCAGAGCCA | 92 | E53SaCas9KKH | TGGAAGCTAAGGAAGAAGC |
| | 22 | AGC | | 23 | TGA |
| 91 | E53SaCas9KKH | TTAGGACAGGCCAGAGCCA | 93 | E53SaCas9KKH | AATGAAATGTTAAAGGATT |
| | 22 | AGC | | 24 | CAA |
| 91 | E53SaCas9KKH | TTAGGACAGGCCAGAGCCA | 94 | E53SaCas9KKH | GCAACAGTTGAATGAAATG |
| | 22 | AGC | | 25 | TTA |
| 91 | E53SaCas9KKH | TTAGGACAGGCCAGAGCCA | 95 | E53SaCas9KKH | AGAACCGGAGGCAACAGTT |
| | 22 | AGC | | 26 | GAA |
| 91 | E53SaCas9KKH | TTAGGACAGGCCAGAGCCA | 96 | E53SaCas9KKH | CCTTCAGAACCGGAGGCAA |
| | 22 | AGC | | 27 | CAG |
| 91 | E53SaCas9KKH | TTAGGACAGGCCAGAGCCA | 97 | E53SaCas9KKH | GAACACCTTCAGAACCGGA |
| | 22 | AGC | | 28 | GGC |
| 91 | E53SaCas9KKH | TTAGGACAGGCCAGAGCCA | 98 | E53SaCas9KKH | AAAGAATTCAGAATCAGTG |
| | 22 | AGC | | 29 | GGA |
| 91 | E53SaCas9KKH | TTAGGACAGGCCAGAGCCA | 99 | E53SaCas9KKH | AGTTGAAAGAATTCAGAAT |
| | 22 | AGC | | 30 | CAG |
| 91 | E53SaCas9KKH | TTAGGACAGGCCAGAGCCA | 100 | E53SaCas9KKH | ATTCTAGTTGAAAGAATTC |
| | 22 | AGC | | 31 | AGA |
| 91 | E53SaCas9KKH | TTAGGACAGGCCAGAGCCA | 101 | E53SaCas9KKH | TTTTATTCTAGTTGAAAGA |
| | 22 | AGC | | 32 | ATT |
| 91 | E53SaCas9KKH | TTAGGACAGGCCAGAGCCA | 102 | E53SaCas9KKH | TTTTTCCTTTTATTCTAGT |
| | 22 | AGC | | 33 | TGA |
| 92 | E53SaCas9KKH | TGGAAGCTAAGGAAGAAGC | 93 | E53SaCas9KKH | AATGAAATGTTAAAGGATT |
| | 23 | TGA | | 24 | CAA |
| 92 | E53SaCas9KKH | TGGAAGCTAAGGAAGAAGC | 94 | E53SaCas9KKH | GCAACAGTTGAATGAAATG |
| | 23 | TGA | | 25 | TTA |
| 92 | E53SaCas9KKH | TGGAAGCTAAGGAAGAAGC | 95 | E53SaCas9KKH | AGAACCGGAGGCAACAGTT |
| | 23 | TGA | | 26 | GAA |
| 92 | E53SaCas9KKH | TGGAAGCTAAGGAAGAAGC | 96 | E53SaCas9KKH | CCTTCAGAACCGGAGGCAA |
| | 23 | TGA | | 27 | CAG |
| 92 | E53SaCas9KKH | TGGAAGCTAAGGAAGAAGC | 97 | E53SaCas9KKH | GAACACCTTCAGAACCGGA |
| | 23 | TGA | | 28 | GGC |
| 92 | E53SaCas9KKH | TGGAAGCTAAGGAAGAAGC | 98 | E53SaCas9KKH | AAAGAATTCAGAATCAGTG |
| | 23 | TGA | | 29 | GGA |
| 92 | E53SaCas9KKH | TGGAAGCTAAGGAAGAAGC | 99 | E53SaCas9KKH | AGTTGAAAGAATTCAGAAT |
| | 23 | TGA | | 30 | CAG |
| 92 | E53SaCas9KKH | TGGAAGCTAAGGAAGAAGC | 100 | E53SaCas9KKH | ATTCTAGTTGAAAGAATTC |
| | 23 | TGA | | 31 | AGA |
| 92 | E53SaCas9KKH | TGGAAGCTAAGGAAGAAGC | 101 | E53SaCas9KKH | TTTTATTCTAGTTGAAAGA |
| | 23 | TGA | | 32 | ATT |
| 92 | E53SaCas9KKH | TGGAAGCTAAGGAAGAAGC | 102 | E53SaCas9KKH | TTTTTCCTTTTATTCTAGT |
| | 23 | TGA | | 33 | TGA |
| 93 | E53SaCas9KKH | AATGAAATGTTAAAGGATT | 94 | E53SaCas9KKH | GCAACAGTTGAATGAAATG |
| | 24 | CAA | | 25 | TTA |
| 93 | E53SaCas9KKH | AATGAAATGTTAAAGGATT | 95 | E53SaCas9KKH | AGAACCGGAGGCAACAGTT |
| | 24 | CAA | | 26 | GAA |
| 93 | E53SaCas9KKH | AATGAAATGTTAAAGGATT | 96 | E53SaCas9KKH | CCTTCAGAACCGGAGGCAA |
| | 24 | CAA | | 27 | CAG |
| 93 | E53SaCas9KKH | AATGAAATGTTAAAGGATT | 97 | E53SaCas9KKH | GAACACCTTCAGAACCGGA |
| | 24 | CAA | | 28 | GGC |
| 93 | E53SaCas9KKH | AATGAAATGTTAAAGGATT | 98 | E53SaCas9KKH | AAAGAATTCAGAATCAGTG |
| | 24 | CAA | | 29 | GGA |
| 93 | E53SaCas9KKH | AATGAAATGTTAAAGGATT | 99 | E53SaCas9KKH | AGTTGAAAGAATTCAGAAT |
| | 24 | CAA | | 30 | CAG |
| 93 | E53SaCas9KKH | AATGAAATGTTAAAGGATT | 100 | E53SaCas9KKH | ATTCTAGTTGAAAGAATTC |
| | 24 | CAA | | 31 | AGA |
| 93 | E53SaCas9KKH | AATGAAATGTTAAAGGATT | 101 | E53SaCas9KKH | TTTTATTCTAGTTGAAAGA |
| | 24 | CAA | | 32 | ATT |
| 93 | E53SaCas9KKH | AATGAAATGTTAAAGGATT | 102 | E53SaCas9KKH | TTTTTCCTTTTATTCTAGT |
| | 24 | CAA | | 33 | TGA |
| 94 | E53SaCas9KKH | GCAACAGTTGAATGAAATG | 95 | E53SaCas9KKH | AGAACCGGAGGCAACAGTT |
| | 25 | TTA | | 26 | GAA |
| 94 | E53SaCas9KKH | GCAACAGTTGAATGAAATG | 96 | E53SaCas9KKH | CCTTCAGAACCGGAGGCAA |
| | 25 | TTA | | 27 | CAG |
| 94 | E53SaCas9KKH | GCAACAGTTGAATGAAATG | 97 | E53SaCas9KKH | GAACACCTTCAGAACCGGA |
| | 25 | TTA | | 28 | GGC |
| 94 | E53SaCas9KKH | GCAACAGTTGAATGAAATG | 98 | E53SaCas9KKH | AAAGAATTCAGAATCAGTG |
| | 25 | TTA | | 29 | GGA |
| 94 | E53SaCas9KKH | GCAACAGTTGAATGAAATG | 99 | E53SaCas9KKH | AGTTGAAAGAATTCAGAAT |
| | 25 | TTA | | 30 | CAG |
| 94 | E53SaCas9KKH | GCAACAGTTGAATGAAATG | 100 | E53SaCas9KKH | ATTCTAGTTGAAAGAATTC |
| | 25 | TTA | | 31 | AGA |
| 94 | E53SaCas9KKH | GCAACAGTTGAATGAAATG | 101 | E53SaCas9KKH | TTTTATTCTAGTTGAAAGA |
| | 25 | TTA | | 32 | ATT |
| 94 | E53SaCas9KKH | GCAACAGTTGAATGAAATG | 102 | E53SaCas9KKH | TTTTTCCTTTTATTCTAGT |
| | 25 | TTA | | 33 | TGA |
| 95 | E53SaCas9KKH | AGAACCGGAGGCAACAGTT | 96 | E53SaCas9KKH | CCTTCAGAACCGGAGGCAA |
| | 26 | GAA | | 27 | CAG |
| 95 | E53SaCas9KKH | AGAACCGGAGGCAACAGTT | 97 | E53SaCas9KKH | GAACACCTTCAGAACCGGA |
| | 26 | GAA | | 28 | GGC |
| 95 | E53SaCas9KKH | AGAACCGGAGGCAACAGTT | 98 | E53SaCas9KKH | AAAGAATTCAGAATCAGTG |
| | 26 | GAA | | 29 | GGA |
| 95 | E53SaCas9KKH | AGAACCGGAGGCAACAGTT | 99 | E53SaCas9KKH | AGTTGAAAGAATTCAGAAT |
| | 26 | GAA | | 30 | CAG |
| 95 | E53SaCas9KKH | AGAACCGGAGGCAACAGTT | 100 | E53SaCas9KKH | ATTCTAGTTGAAAGAATTC |
| | 26 | GAA | | 31 | AGA |
| 95 | E53SaCas9KKH | AGAACCGGAGGCAACAGTT | 101 | E53SaCas9KKH | TTTTATTCTAGTTGAAAGA |
| | 26 | GAA | | 32 | ATT |
| 95 | E53SaCas9KKH | AGAACCGGAGGCAACAGTT | 102 | E53SaCas9KKH | TTTTTCCTTTTATTCTAGT |
| | 26 | GAA | | 33 | TGA |
| 96 | E53SaCas9KKH | CCTTCAGAACCGGAGGCAA | 97 | E53SaCas9KKH | GAACACCTTCAGAACCGGA |
| | 27 | CAG | | 28 | GGC |
| 96 | E53SaCas9KKH | CCTTCAGAACCGGAGGCAA | 98 | E53SaCas9KKH | AAAGAATTCAGAATCAGTG |
| | 27 | CAG | | 29 | GGA |
| 96 | E53SaCas9KKH | CCTTCAGAACCGGAGGCAA | 99 | E53SaCas9KKH | AGTTGAAAGAATTCAGAAT |
| | 27 | CAG | | 30 | CAG |
| 96 | E53SaCas9KKH | CCTTCAGAACCGGAGGCAA | 100 | E53SaCas9KKH | ATTCTAGTTGAAAGAATTC |
| | 27 | CAG | | 31 | AGA |
| 96 | E53SaCas9KKH | CCTTCAGAACCGGAGGCAA | 101 | E53SaCas9KKH | TTTTATTCTAGTTGAAAGA |
| | 27 | CAG | | 32 | ATT |
| 96 | E53SaCas9KKH | CCTTCAGAACCGGAGGCAA | 102 | E53SaCas9KKH | TTTTTCCTTTTATTCTAGT |
| | 27 | CAG | | 33 | TGA |
| 97 | E53SaCas9KKH | GAACACCTTCAGAACCGGA | 98 | E53SaCas9KKH | AAAGAATTCAGAATCAGTG |
| | 28 | GGC | | 29 | GGA |
| 97 | E53SaCas9KKH | GAACACCTTCAGAACCGGA | 99 | E53SaCas9KKH | AGTTGAAAGAATTCAGAAT |
| | 28 | GGC | | 30 | CAG |
| 97 | E53SaCas9KKH | GAACACCTTCAGAACCGGA | 100 | E53SaCas9KKH | ATTCTAGTTGAAAGAATTC |
| | 28 | GGC | | 31 | AGA |
| 97 | E53SaCas9KKH | GAACACCTTCAGAACCGGA | 101 | E53SaCas9KKH | TTTTATTCTAGTTGAAAGA |
| | 28 | GGC | | 32 | ATT |
| 97 | E53SaCas9KKH | GAACACCTTCAGAACCGGA | 102 | E53SaCas9KKH | TTTTTCCTTTTATTCTAGT |
| | 28 | GGC | | 33 | TGA |
| 98 | E53SaCas9KKH | AAAGAATTCAGAATCAGTG | 99 | E53SaCas9KKH | AGTTGAAAGAATTCAGAAT |
| | 29 | GGA | | 30 | CAG |
| 98 | E53SaCas9KKH | AAAGAATTCAGAATCAGTG | 100 | E53SaCas9KKH | ATTCTAGTTGAAAGAATTC |
| | 29 | GGA | | 31 | AGA |
| 98 | E53SaCas9KKH | AAAGAATTCAGAATCAGTG | 101 | E53SaCas9KKH | TTTTATTCTAGTTGAAAGA |
| | 29 | GGA | | 32 | ATT |
| 98 | E53SaCas9KKH | AAAGAATTCAGAATCAGTG | 102 | E53SaCas9KKH | TTTTTCCTTTTATTCTAGT |
| | 29 | GGA | | 33 | TGA |
| 99 | E53SaCas9KKH | AGTTGAAAGAATTCAGAAT | 100 | E53SaCas9KKH | ATTCTAGTTGAAAGAATTC |
| | 30 | CAG | | 31 | AGA |
| 99 | E53SaCas9KKH | AGTTGAAAGAATTCAGAAT | 101 | E53SaCas9KKH | TTTTATTCTAGTTGAAAGA |
| | 30 | CAG | | 32 | ATT |
| 99 | E53SaCas9KKH | AGTTGAAAGAATTCAGAAT | 102 | E53SaCas9KKH | TTTTTCCTTTTATTCTAGT |
| | 30 | CAG | | 33 | TGA |
| 100 | E53SaCas9KKH | ATTCTAGTTGAAAGAATTC | 101 | E53SaCas9KKH | TTTTATTCTAGTTGAAAGA |
| | 31 | AGA | | 32 | ATT |
| 100 | E53SaCas9KKH | ATTCTAGTTGAAAGAATTC | 102 | E53SaCas9KKH | TTTTTCCTTTTATTCTAGT |
| | 31 | AGA | | 33 | TGA |
| 101 | E53SaCas9KKH | TTTTATTCTAGTTGAAAGA | 102 | E53SaCas9KKH | TTTTTCCTTTTATTCTAGT |
| | 32 | ATT | | 33 | TGA |

TABLE 5C
(3). Exon 53 SluCas9 guides:

| Seq | Guide ID or | |
|---|---|---|
| ID No. | Guide RNA Name | Guide Sequence |
| 270 | E53Slu2 | CCTTGGTTTCTGTGATTTTCTT |
| 271 | E53Slu3 | CTTTTGGATTGCATCTACTGTA |
| 272 | E53Slu4 | TTTTGGATTGCATCTACTGTAT |
| 273 | E53Slu5 | ACCCTCCTTCCATGACTCAAGC |
| 274 | E53Slu6 | CTTCCATGACTCAAGCTTGGCT |
| 275 | E53Slu7 | ACATTTCATTCAACTGTTGCCT |
| 276 | E53Slu8 | TCAACTGTTGCCTCCGGTTCTG |
| 277 | E53Slu10 | CCAAAAGAAAATCACAGAAACC |
| 278 | E53Slu11 | GCCAAGCTTGAGTCATGGAAGG |
| 279 | E53Slu12 | AGCCAAGCTTGAGTCATGGAAG |
| 280 | E53Slu13 | CAGAGCCAAGCTTGAGTCATGG |
| 281 | E53Slu14 | AGGCCAGAGCCAAGCTTGAGTC |
| 282 | E53Slu15 | AGAAGCTGAGCAGGTCTTAGGA |
| 283 | E53Slu16 | AAGGAAGAAGCTGAGCAGGTCT |
| 284 | E53Slu17 | GGAAGCTAAGGAAGAAGCTGAG |
| 285 | E53Slu18 | TTCAACACAATGGCTGGAAGCT |
| 286 | E53Slu19 | GTTAAAGGATTCAACACAATGG |
| 287 | E53Slu20 | AAATGTTAAAGGATTCAACACA |
| 288 | E53Slu21 | GCAACAGTTGAATGAAATGTTA |
| 289 | E53Slu22 | TACAAGAACACCTTCAGAACCG |
| 290 | E53Slu23 | AAGTACAAGAACACCTTCAGAA |
| 291 | E53Slu24 | AGTTGAAAGAATTCAGAATCAG |
| 292 | E53Slu25 | TAGTTGAAAGAATTCAGAATCA |
| 924 | E53SL3.1A-Cv5 | CATTTGGATTGCATCTACTGTA |
| 925 | E53SL3.1C-Cv5 | CCTTTGGATTGCATCTACTGTA |
| 926 | E53S13.1G-Cv5 | CGTTTGGATTGCATCTACTGTA |
| 927 | E53S13.2A-Cv5 | CTATTGGATTGCATCTACTGTA |
| 928 | E53S13.2C-Cv5 | CTCTTGGATTGCATCTACTGTA |
| 929 | E53S13.2G-Cv5 | CTGTTGGATTGCATCTACTGTA |
| 930 | E53S13.3A-Cv5 | CTTATGGATTGCATCTACTGTA |
| 931 | E53S13.3C-Cv5 | CTTCTGGATTGCATCTACTGTA |
| 932 | E53S13.3G-Cv5 | CTTGTGGATTGCATCTACTGTA |
| 933 | E53S13.4A-Cv5 | CTTTAGGATTGCATCTACTGTA |
| 934 | E53S13.4C-Cv5 | CTTTCGGATTGCATCTACTGTA |
| 935 | E53S13.4G-Cv5 | CTTTGGGATTGCATCTACTGTA |
| 936 | E53S13.del2-Cv5 | TTTGGATTGCATCTACTGTA |
| 937 | E53S13.delT-Cv5 | CTTTGGATTGCATCTACTGTA |
| 938 | E53S13.delT + A.- | ACTTTGGATTGCATCTACTGTA |
| | Cv5 | |
| 950 | E53SL3-21 | TTTTGGATTGCATCTACTGTA |
| 951 | E53SL3-20 | TTTGGATTGCATCTACTGTA |
| 952 | E53SL3-19 | TTGGATTGCATCTACTGTA |
| 953 | E53SL3-18 | TGGATTGCATCTACTGTA |
| 954 | E53SL3-17 | GGATTGCATCTACTGTA |
| 955 | E53SL3-16 | GATTGCATCTACTGTA |

TABLE 5D
(4). Exon 53 SluCas9 pairs:

| Guide 1 | | | Guide 2 | | |
|---|---|---|---|---|---|
| Seq ID | | | Seq ID | | |
| No. | Guide 1 | Guide 1 Sequence | No. | Guide 2 | Guide 2 Sequence |
| 270 | E53SL2 | CCTTGGTTTCTGTGATTTT | 271 | E53SL3 | CTTTTGGATTGCATCTACT |
| | | CTT | | | GTA |
| 270 | E53SL2 | CCTTGGTTTCTGTGATTTT | 272 | E53SL4 | TTTTGGATTGCATCTACTG |
| | | CTT | | | TAT |
| 270 | E53SL2 | CCTTGGTTTCTGTGATTTT | 273 | E53SL5 | ACCCTCCTTCCATGACTCA |
| | | CTT | | | AGC |
| 270 | E53SL2 | CCTTGGTTTCTGTGATTTT | 274 | E53SL6 | CTTCCATGACTCAAGCTTG |
| | | CTT | | | GCT |
| 270 | E53SL2 | CCTTGGTTTCTGTGATTTT | 275 | E53SL7 | ACATTTCATTCAACTGTTG |
| | | CTT | | | CCT |
| 270 | E53SL2 | CCTTGGTTTCTGTGATTTT | 276 | E53SL8 | TCAACTGTTGCCTCCGGTT |
| | | CTT | | | CTG |
| 270 | E53SL2 | CCTTGGTTTCTGTGATTTT | 277 | E53SL10 | CCAAAAGAAAATCACAGAA |
| | | CTT | | | ACC |
| 270 | E53SL2 | CCTTGGTTTCTGTGATTTT | 278 | E53SL1 | GCCAAGCTTGAGTCATGGA |
| | | CTT | | 1 | AGG |
| 270 | E53SL2 | CCTTGGTTTCTGTGATTTT | 279 | E53SL1 | AGCCAAGCTTGAGTCATGG |
| | | CTT | | 2 | AAG |
| 270 | E53SL2 | CCTTGGTTTCTGTGATTTT | 280 | E53SL1 | CAGAGCCAAGCTTGAGTCA |
| | | CTT | | 3 | TGG |
| 270 | E53SL2 | CCTTGGTTTCTGTGATTTT | 281 | E53SL1 | AGGCCAGAGCCAAGCTTGA |
| | | CTT | | 4 | GTC |
| 270 | E53SL2 | CCTTGGTTTCTGTGATTTT | 282 | E53SL1 | AGAAGCTGAGCAGGTCTTA |
| | | CTT | | 5 | GGA |
| 270 | E53SL2 | CCTTGGTTTCTGTGATTTT | 283 | E53SL1 | AAGGAAGAAGCTGAGCAGG |
| | | CTT | | 6 | TCT |
| 270 | E53SL2 | CCTTGGTTTCTGTGATTTT | 284 | E53SL1 | GGAAGCTAAGGAAGAAGCT |
| | | CTT | | 7 | GAG |
| 270 | E53SL2 | CCTTGGTTTCTGTGATTTT | 285 | E53SL1 | TTCAACACAATGGCTGGAA |
| | | CTT | | 8 | GCT |
| 270 | E53SL2 | CCTTGGTTTCTGTGATTTT | 286 | E53SL1 | GTTAAAGGATTCAACACAA |
| | | CTT | | 9 | TGG |
| 270 | E53SL2 | CCTTGGTTTCTGTGATTTT | 287 | E53SL2 | AAATGTTAAAGGATTCAAC |
| | | CTT | | 0 | ACA |
| 270 | E53SL2 | CCTTGGTTTCTGTGATTTT | 288 | E53SL2 | GCAACAGTTGAATGAAATG |
| | | CTT | | 1 | TTA |
| 270 | E53SL2 | CCTTGGTTTCTGTGATTTT | 289 | E53SL2 | TACAAGAACACCTTCAGAA |
| | | CTT | | 2 | CCG |
| 270 | E53SL2 | CCTTGGTTTCTGTGATTTT | 290 | E53SL2 | AAGTACAAGAACACCTTCA |
| | | CTT | | 3 | GAA |
| 270 | E53SL2 | CCTTGGTTTCTGTGATTTT | 291 | E53SL2 | AGTTGAAAGAATTCAGAAT |
| | | CTT | | 4 | CAG |
| 270 | E53SL2 | CCTTGGTTTCTGTGATTTT | 292 | E53SL2 | TAGTTGAAAGAATTCAGAA |
| | | CTT | | 5 | TCA |
| 271 | E53SL3 | CTTTTGGATTGCATCTACT | 272 | E53SL4 | TTTTGGATTGCATCTACTG |
| | | GTA | | | TAT |
| 271 | E53SL3 | CTTTTGGATTGCATCTACT | 273 | E53SL5 | ACCCTCCTTCCATGACTCA |
| | | GTA | | | AGC |
| 271 | E53SL3 | CTTTTGGATTGCATCTACT | 274 | E53SL6 | CTTCCATGACTCAAGCTTG |
| | | GTA | | | GCT |
| 271 | E53SL3 | CTTTTGGATTGCATCTACT | 275 | E53SL7 | ACATTTCATTCAACTGTTG |
| | | GTA | | | CCT |
| 271 | E53SL3 | CTTTTGGATTGCATCTACT | 276 | E53SL8 | TCAACTGTTGCCTCCGGTT |
| | | GTA | | | CTG |
| 271 | E53SL3 | CTTTTGGATTGCATCTACT | 277 | E53SL1 | CCAAAAGAAAATCACAGAA |
| | | GTA | | 0 | ACC |
| 271 | E53SL3 | CTTTTGGATTGCATCTACT | 278 | E53SL1 | GCCAAGCTTGAGTCATGGA |
| | | GTA | | 1 | AGG |
| 271 | E53SL3 | CTTTTGGATTGCATCTACT | 279 | E53SL1 | AGCCAAGCTTGAGTCATGG |
| | | GTA | | 2 | AAG |
| 271 | E53SL3 | CTTTTGGATTGCATCTACT | 280 | E53SL1 | CAGAGCCAAGCTTGAGTCA |
| | | GTA | | 3 | TGG |
| 271 | E53SL3 | CTTTTGGATTGCATCTACT | 281 | E53SL1 | AGGCCAGAGCCAAGCTTGA |
| | | GTA | | 4 | GTC |
| 271 | E53SL3 | CTTTTGGATTGCATCTACT | 282 | E53SL1 | AGAAGCTGAGCAGGTCTTA |
| | | GTA | | 5 | GGA |
| 271 | E53SL3 | CTTTTGGATTGCATCTACT | 283 | E53SL1 | AAGGAAGAAGCTGAGCAGG |
| | | GTA | | 6 | TCT |
| 271 | E53SL3 | CTTTTGGATTGCATCTACT | 284 | E53SL1 | GGAAGCTAAGGAAGAAGCT |
| | | GTA | | 7 | GAG |
| 271 | E53SL3 | CTTTTGGATTGCATCTACT | 285 | E53SL1 | TTCAACACAATGGCTGGAA |
| | | GTA | | 8 | GCT |
| 271 | E53SL3 | CTTTTGGATTGCATCTACT | 286 | E53SL1 | GTTAAAGGATTCAACACAA |
| | | GTA | | 9 | TGG |
| 271 | E53SL3 | CTTTTGGATTGCATCTACT | 287 | E53SL2 | AAATGTTAAAGGATTCAAC |
| | | GTA | | 0 | ACA |
| 271 | E53SL3 | CTTTTGGATTGCATCTACT | 288 | E53SL2 | GCAACAGTTGAATGAAATG |
| | | GTA | | 1 | TTA |
| 271 | E53SL3 | CTTTTGGATTGCATCTACT | 289 | E53SL2 | TACAAGAACACCTTCAGAA |
| | | GTA | | 2 | CCG |
| 271 | E53SL3 | CTTTTGGATTGCATCTACT | 290 | E53SL2 | AAGTACAAGAACACCTTCA |
| | | GTA | | 3 | GAA |
| 271 | E53SL3 | CTTTTGGATTGCATCTACT | 291 | E53SL2 | AGTTGAAAGAATTCAGAAT |
| | | GTA | | 4 | CAG |
| 271 | E53SL3 | CTTTTGGATTGCATCTACT | 292 | E53SL2 | TAGTTGAAAGAATTCAGAA |
| | | GTA | | 5 | TCA |
| 272 | E53SL4 | TTTTGGATTGCATCTACTG | 273 | E53SL5 | ACCCTCCTTCCATGACTCA |
| | | TAT | | | AGC |
| 272 | E53SL4 | TTTTGGATTGCATCTACTG | 274 | E53SL6 | CTTCCATGACTCAAGCTTG |
| | | TAT | | | GCT |
| 272 | E53SL4 | TTTTGGATTGCATCTACTG | 275 | E53SL7 | ACATTTCATTCAACTGTTG |
| | | TAT | | | CCT |
| 272 | E53SL4 | TTTTGGATTGCATCTACTG | 276 | E53SL8 | TCAACTGTTGCCTCCGGTT |
| | | TAT | | | CTG |
| 272 | E53SL4 | TTTTGGATTGCATCTACTG | 277 | E53SL1 | CCAAAAGAAAATCACAGAA |
| | | TAT | | 0 | ACC |
| 272 | E53SL4 | TTTTGGATTGCATCTACTG | 278 | E53SL1 | GCCAAGCTTGAGTCATGGA |
| | | TAT | | 1 | AGG |
| 272 | E53SL4 | TTTTGGATTGCATCTACTG | 279 | E53SL1 | AGCCAAGCTTGAGTCATGG |
| | | TAT | | 2 | AAG |
| 272 | E53SL4 | TTTTGGATTGCATCTACTG | 280 | E53SL1 | CAGAGCCAAGCTTGAGTCA |
| | | TAT | | 3 | TGG |
| 272 | E53SL4 | TTTTGGATTGCATCTACTG | 281 | E53SL1 | AGGCCAGAGCCAAGCTTGA |
| | | TAT | | 4 | GTC |
| 272 | E53SL4 | TTTTGGATTGCATCTACTG | 282 | E53SL1 | AGAAGCTGAGCAGGTCTTA |
| | | TAT | | 5 | GGA |
| 272 | E53SL4 | TTTTGGATTGCATCTACTG | 283 | E53SL1 | AAGGAAGAAGCTGAGCAGG |
| | | TAT | | 6 | TCT |
| 272 | E53SL4 | TTTTGGATTGCATCTACTG | 284 | E53SL1 | GGAAGCTAAGGAAGAAGCT |
| | | TAT | | 7 | GAG |
| 272 | E53SL4 | TTTTGGATTGCATCTACTG | 285 | E53SL1 | TTCAACACAATGGCTGGAA |
| | | TAT | | 8 | GCT |
| 272 | E53SL4 | TTTTGGATTGCATCTACTG | 286 | E53SL1 | GTTAAAGGATTCAACACAA |
| | | TAT | | 9 | TGG |
| 272 | E53SL4 | TTTTGGATTGCATCTACTG | 287 | E53SL2 | AAATGTTAAAGGATTCAAC |
| | | TAT | | 0 | ACA |
| 272 | E53SL4 | TTTTGGATTGCATCTACTG | 288 | E53SL2 | GCAACAGTTGAATGAAATG |
| | | TAT | | 1 | TTA |
| 272 | E53SL4 | TTTTGGATTGCATCTACTG | 289 | E53SL2 | TACAAGAACACCTTCAGAA |
| | | TAT | | 2 | CCG |
| 272 | E53SL4 | TTTTGGATTGCATCTACTG | 290 | E53SL2 | AAGTACAAGAACACCTTCA |
| | | TAT | | 3 | GAA |
| 272 | E53SL4 | TTTTGGATTGCATCTACTG | 291 | E53SL2 | AGTTGAAAGAATTCAGAAT |
| | | TAT | | 4 | CAG |
| 272 | E53SL4 | TTTTGGATTGCATCTACTG | 292 | E53SL2 | TAGTTGAAAGAATTCAGAA |
| | | TAT | | 5 | TCA |
| 273 | E53SL5 | ACCCTCCTTCCATGACTCA | 274 | E53SL6 | CTTCCATGACTCAAGCTTG |
| | | AGC | | | GCT |
| 273 | E53SL5 | ACCCTCCTTCCATGACTCA | 275 | E53SL7 | ACATTTCATTCAACTGTTG |
| | | AGC | | | CCT |
| 273 | E53SL5 | ACCCTCCTTCCATGACTCA | 276 | E53SL8 | TCAACTGTTGCCTCCGGTT |
| | | AGC | | | CTG |
| 273 | E53SL5 | ACCCTCCTTCCATGACTCA | 277 | E53SL1 | CCAAAAGAAAATCACAGAA |
| | | AGC | | 0 | ACC |
| 273 | E53SL5 | ACCCTCCTTCCATGACTCA | 278 | E53SL1 | GCCAAGCTTGAGTCATGGA |
| | | AGC | | 1 | AGG |
| 273 | E53SL5 | ACCCTCCTTCCATGACTCA | 279 | E53SL1 | AGCCAAGCTTGAGTCATGG |
| | | AGC | | 2 | AAG |
| 273 | E53SL5 | ACCCTCCTTCCATGACTCA | 280 | E53SL1 | CAGAGCCAAGCTTGAGTCA |
| | | AGC | | 3 | TGG |
| 273 | E53SL5 | ACCCTCCTTCCATGACTCA | 281 | E53SL1 | AGGCCAGAGCCAAGCTTGA |
| | | AGC | | 1 | GTC |
| 273 | E53SL5 | ACCCTCCTTCCATGACTCA | 282 | E53SL1 | AGAAGCTGAGCAGGTCTTA |
| | | AGC | | 5 | GGA |
| 273 | E53SL5 | ACCCTCCTTCCATGACTCA | 283 | E53SL1 | AAGGAAGAAGCTGAGCAGG |
| | | AGC | | 6 | TCT |
| 273 | E53SL5 | ACCCTCCTTCCATGACTCA | 284 | E53SL1 | GGAAGCTAAGGAAGAAGCT |
| | | AGC | | 7 | GAG |
| 273 | E53SL5 | ACCCTCCTTCCATGACTCA | 285 | E53SL1 | TTCAACACAATGGCTGGAA |
| | | AGC | | 8 | GCT |
| 273 | E53SL5 | ACCCTCCTTCCATGACTCA | 286 | E53SL1 | GTTAAAGGATTCAACACAA |
| | | AGC | | 9 | TGG |
| 273 | E53SL5 | ACCCTCCTTCCATGACTCA | 287 | E53SL2 | AAATGTTAAAGGATTCAAC |
| | | AGC | | 0 | ACA |
| 273 | E53SL5 | ACCCTCCTTCCATGACTCA | 288 | E53SL2 | GCAACAGTTGAATGAAATG |
| | | AGC | | 1 | TTA |
| 273 | E53SL5 | ACCCTCCTTCCATGACTCA | 289 | E53SL2 | TACAAGAACACCTTCAGAA |
| | | AGC | | 2 | CCG |
| 273 | E53SL5 | ACCCTCCTTCCATGACTCA | 290 | E53SL2 | AAGTACAAGAACACCTTCA |
| | | AGC | | 3 | GAA |
| 273 | E53SL5 | ACCCTCCTTCCATGACTCA | 291 | E53SL2 | AGTTGAAAGAATTCAGAAT |
| | | AGC | | 4 | CAG |
| 273 | E53SL5 | ACCCTCCTTCCATGACTCA | 292 | E53SL2 | TAGTTGAAAGAATTCAGAA |
| | | AGC | | 5 | TCA |
| 274 | E53SL6 | CTTCCATGACTCAAGCTTG | 275 | E53SL7 | ACATTTCATTCAACTGTTG |
| | | GCT | | | CCT |
| 274 | E53SL6 | CTTCCATGACTCAAGCTTG | 276 | E53SL8 | TCAACTGTTGCCTCCGGTT |
| | | GCT | | | CTG |
| 274 | E53SL6 | CTTCCATGACTCAAGCTTG | 277 | E53SL1 | CCAAAAGAAAATCACAGAA |
| | | GCT | | 0 | ACC |
| 274 | E53SL6 | CTTCCATGACTCAAGCTTG | 278 | E53SL1 | GCCAAGCTTGAGTCATGGA |
| | | GCT | | 1 | AGG |
| 274 | E53SL6 | CTTCCATGACTCAAGCTTG | 279 | E53SL1 | AGCCAAGCTTGAGTCATGG |
| | | GCT | | 2 | AAG |
| 274 | E53SL6 | CTTCCATGACTCAAGCTTG | 280 | E53SL1 | CAGAGCCAAGCTTGAGTCA |
| | | GCT | | 3 | TGG |
| 274 | E53SL6 | CTTCCATGACTCAAGCTTG | 281 | E53SL1 | AGGCCAGAGCCAAGCTTGA |
| | | GCT | | 4 | GTC |
| 274 | E53SL6 | CTTCCATGACTCAAGCTTG | 282 | E53SL1 | AGAAGCTGAGCAGGTCTTA |
| | | GCT | | 5 | GGA |
| 274 | E53SL6 | CTTCCATGACTCAAGCTTG | 283 | E53SL1 | AAGGAAGAAGCTGAGCAGG |
| | | GCT | | 6 | TCT |
| 274 | E53SL6 | CTTCCATGACTCAAGCTTG | 284 | E53SL1 | GGAAGCTAAGGAAGAAGCT |
| | | GCT | | 7 | GAG |
| 274 | E53SL6 | CTTCCATGACTCAAGCTTG | 285 | E53SL1 | TTCAACACAATGGCTGGAA |
| | | GCT | | 8 | GCT |
| 274 | E53SL6 | CTTCCATGACTCAAGCTTG | 286 | E53SL1 | GTTAAAGGATTCAACACAA |
| | | GCT | | 9 | TGG |
| 274 | E53SL6 | CTTCCATGACTCAAGCTTG | 287 | E53SL2 | AAATGTTAAAGGATTCAAC |
| | | GCT | | 0 | ACA |
| 274 | E53SL6 | CTTCCATGACTCAAGCTTG | 288 | E53SL2 | GCAACAGTTGAATGAAATG |
| | | GCT | | 1 | TTA |
| 274 | E53SL6 | CTTCCATGACTCAAGCTTG | 289 | E53SL2 | TACAAGAACACCTTCAGAA |
| | | GCT | | 2 | CCG |
| 274 | E53SL6 | CTTCCATGACTCAAGCTTG | 290 | E53SL2 | AAGTACAAGAACACCTTCA |
| | | GCT | | 3 | GAA |
| 274 | E53SL6 | CTTCCATGACTCAAGCTTG | 291 | E53SL2 | AGTTGAAAGAATTCAGAAT |
| | | GCT | | 4 | CAG |
| 274 | E53SL6 | CTTCCATGACTCAAGCTTG | 292 | E53SL2 | TAGTTGAAAGAATTCAGAA |
| | | GCT | | 5 | TCA |
| 275 | E53SL7 | ACATTTCATTCAACTGTTG | 276 | E53SL8 | TCAACTGTTGCCTCCGGTT |
| | | CCT | | | CTG |
| 275 | E53SL7 | ACATTTCATTCAACTGTTG | 277 | E53SL1 | CCAAAAGAAAATCACAGAA |
| | | CCT | | 0 | ACC |
| 275 | E53SL7 | ACATTTCATTCAACTGTTG | 278 | E53SL1 | GCCAAGCTTGAGTCATGGA |
| | | CCT | | 1 | AGG |
| 275 | E53SL7 | ACATTTCATTCAACTGTTG | 279 | E53SL1 | AGCCAAGCTTGAGTCATGG |
| | | CCT | | 2 | AAG |
| 275 | E53SL7 | ACATTTCATTCAACTGTTG | 280 | E53SL1 | CAGAGCCAAGCTTGAGTCA |
| | | CCT | | 3 | TGG |
| 275 | E53SL7 | ACATTTCATTCAACTGTTG | 281 | E53SL1 | AGGCCAGAGCCAAGCTTGA |
| | | CCT | | 4 | GTC |
| 275 | E53SL7 | ACATTTCATTCAACTGTTG | 282 | E53SL1 | AGAAGCTGAGCAGGTCTTA |
| | | CCT | | 5 | GGA |
| 275 | E53SL7 | ACATTTCATTCAACTGTTG | 283 | E53SL1 | AAGGAAGAAGCTGAGCAGG |
| | | CCT | | 6 | TCT |
| 275 | E53SL7 | ACATTTCATTCAACTGTTG | 284 | E53SL1 | GGAAGCTAAGGAAGAAGCT |
| | | CCT | | 7 | GAG |
| 275 | E53SL7 | ACATTTCATTCAACTGTTG | 285 | E53SL1 | TTCAACACAATGGCTGGAA |
| | | CCT | | 8 | GCT |
| 275 | E53SL7 | ACATTTCATTCAACTGTTG | 286 | E53SL1 | GTTAAAGGATTCAACACAA |
| | | CCT | | 9 | TGG |
| 275 | E53SL7 | ACATTTCATTCAACTGTTG | 287 | E53SL2 | AAATGTTAAAGGATTCAAC |
| | | CCT | | 0 | ACA |
| 275 | E53SL7 | ACATTTCATTCAACTGTTG | 288 | E53SL2 | GCAACAGTTGAATGAAATG |
| | | CCT | | 1 | TTA |
| 275 | E53SL7 | ACATTTCATTCAACTGTTG | 289 | E53SL2 | TACAAGAACACCTTCAGAA |
| | | CCT | | 2 | CCG |
| 275 | E53SL7 | ACATTTCATTCAACTGTTG | 290 | E53SL2 | AAGTACAAGAACACCTTCA |
| | | CCT | | 3 | GAA |
| 275 | E53SL7 | ACATTTCATTCAACTGTTG | 291 | E53SL2 | AGTTGAAAGAATTCAGAAT |
| | | CCT | | 4 | CAG |
| 275 | E53SL7 | ACATTTCATTCAACTGTTG | 292 | E53SL2 | TAGTTGAAAGAATTCAGAA |
| | | CCT | | 5 | TCA |
| 276 | E53SL8 | TCAACTGTTGCCTCCGGTT | 277 | E53SL1 | CCAAAAGAAAATCACAGAA |
| | | CTG | | 0 | ACC |
| 276 | E53SL8 | TCAACTGTTGCCTCCGGTT | 278 | E53SL1 | GCCAAGCTTGAGTCATGGA |
| | | CTG | | 1 | AGG |
| 276 | E53SL8 | TCAACTGTTGCCTCCGGTT | 279 | E53SL1 | AGCCAAGCTTGAGTCATGG |
| | | CTG | | 2 | AAG |
| 276 | E53SL8 | TCAACTGTTGCCTCCGGTT | 280 | E53SL1 | CAGAGCCAAGCTTGAGTCA |
| | | CTG | | 3 | TGG |
| 276 | E53SL8 | TCAACTGTTGCCTCCGGTT | 281 | E53SL1 | AGGCCAGAGCCAAGCTTGA |
| | | CTG | | 4 | GTC |
| 276 | E53SL8 | TCAACTGTTGCCTCCGGTT | 282 | E53SL1 | AGAAGCTGAGCAGGTCTTA |
| | | CTG | | 5 | GGA |
| 276 | E53SL8 | TCAACTGTTGCCTCCGGTT | 283 | E53SL1 | AAGGAAGAAGCTGAGCAGG |
| | | CTG | | 6 | TCT |
| 276 | E53SL8 | TCAACTGTTGCCTCCGGTT | 284 | E53SL1 | GGAAGCTAAGGAAGAAGCT |
| | | CTG | | 7 | GAG |
| 276 | E53SL8 | TCAACTGTTGCCTCCGGTT | 285 | E53SL1 | TTCAACACAATGGCTGGAA |
| | | CTG | | 8 | GCT |
| 276 | E53SL8 | TCAACTGTTGCCTCCGGTT | 286 | E53SL1 | GTTAAAGGATTCAACACAA |
| | | CTG | | 9 | TGG |
| 276 | E53SL8 | TCAACTGTTGCCTCCGGTT | 287 | E53SL2 | AAATGTTAAAGGATTCAAC |
| | | CTG | | 0 | ACA |
| 276 | E53SL8 | TCAACTGTTGCCTCCGGTT | 288 | E53SL2 | GCAACAGTTGAATGAAATG |
| | | CTG | | 1 | TTA |
| 276 | E53SL8 | TCAACTGTTGCCTCCGGTT | 289 | E53SL2 | TACAAGAACACCTTCAGAA |
| | | CTG | | 2 | CCG |
| 276 | E53SL8 | TCAACTGTTGCCTCCGGTT | 290 | E53SL2 | AAGTACAAGAACACCTTCA |
| | | CTG | | 3 | GAA |
| 276 | E53SL8 | TCAACTGTTGCCTCCGGTT | 291 | E53SL2 | AGTTGAAAGAATTCAGAAT |
| | | CTG | | 4 | CAG |
| 276 | E53SL8 | TCAACTGTTGCCTCCGGTT | 292 | E53SL2 | TAGTTGAAAGAATTCAGAA |
| | | CTG | | 5 | TCA |
| 277 | E53SL10 | CCAAAAGAAAATCACAGAA | 278 | E53SL1 | GCCAAGCTTGAGTCATGGA |
| | | ACC | | 1 | AGG |
| 277 | E53SL10 | CCAAAAGAAAATCACAGAA | 279 | E53SL1 | AGCCAAGCTTGAGTCATGG |
| | | ACC | | 2 | AAG |
| 277 | E53SL10 | CCAAAAGAAAATCACAGAA | 280 | E53SL1 | CAGAGCCAAGCTTGAGTCA |
| | | ACC | | 3 | TGG |
| 277 | E53SL10 | CCAAAAGAAAATCACAGAA | 281 | E53SL1 | AGGCCAGAGCCAAGCTTGA |
| | | ACC | | 4 | GTC |
| 277 | E53SL10 | CCAAAAGAAAATCACAGAA | 282 | E53SL1 | AGAAGCTGAGCAGGTCTTA |
| | | ACC | | 5 | GGA |
| 277 | E53SL10 | CCAAAAGAAAATCACAGAA | 283 | E53SL1 | AAGGAAGAAGCTGAGCAGG |
| | | ACC | | 6 | TCT |
| 277 | E53SL10 | CCAAAAGAAAATCACAGAA | 284 | E53SL1 | GGAAGCTAAGGAAGAAGCT |
| | | ACC | | 7 | GAG |
| 277 | E53SL10 | CCAAAAGAAAATCACAGAA | 285 | E53SL1 | TTCAACACAATGGCTGGAA |
| | | ACC | | 8 | GCT |
| 277 | E53SL10 | CCAAAAGAAAATCACAGAA | 286 | E53SL1 | GTTAAAGGATTCAACACAA |
| | | ACC | | 9 | TGG |
| 277 | E53SL10 | CCAAAAGAAAATCACAGAA | 287 | E53SL2 | AAATGTTAAAGGATTCAAC |
| | | ACC | | 0 | ACA |
| 277 | E53SL10 | CCAAAAGAAAATCACAGAA | 288 | E53SL2 | GCAACAGTTGAATGAAATG |
| | | ACC | | 1 | TTA |
| 277 | E53SL10 | CCAAAAGAAAATCACAGAA | 289 | E53SL2 | TACAAGAACACCTTCAGAA |
| | | ACC | | 2 | CCG |
| 277 | E53SL10 | CCAAAAGAAAATCACAGAA | 290 | E53SL2 | AAGTACAAGAACACCTTCA |
| | | ACC | | 3 | GAA |
| 277 | E53SL10 | CCAAAAGAAAATCACAGAA | 291 | E53SL2 | AGTTGAAAGAATTCAGAAT |
| | | ACC | | 4 | CAG |
| 277 | E53SL10 | CCAAAAGAAAATCACAGAA | 292 | E53SL2 | TAGTTGAAAGAATTCAGAA |
| | | ACC | | 5 | TCA |
| 278 | E53SL11 | GCCAAGCTTGAGTCATGGA | 279 | E53SL1 | AGCCAAGCTTGAGTCATGG |
| | | AGG | | 2 | AAG |
| 278 | E53SL11 | GCCAAGCTTGAGTCATGGA | 280 | E53SL1 | CAGAGCCAAGCTTGAGTCA |
| | | AGG | | 3 | TGG |
| 278 | E53SL11 | GCCAAGCTTGAGTCATGGA | 281 | E53SL1 | AGGCCAGAGCCAAGCTTGA |
| | | AGG | | 4 | GTC |
| 278 | E53SL11 | GCCAAGCTTGAGTCATGGA | 282 | E53SL1 | AGAAGCTGAGCAGGTCTTA |
| | | AGG | | 5 | GGA |
| 278 | E53SL11 | GCCAAGCTTGAGTCATGGA | 283 | E53SL1 | AAGGAAGAAGCTGAGCAGG |
| | | AGG | | 6 | TCT |
| 278 | E53SL11 | GCCAAGCTTGAGTCATGGA | 284 | E53SL1 | GGAAGCTAAGGAAGAAGCT |
| | | AGG | | 7 | GAG |
| 278 | E53SL11 | GCCAAGCTTGAGTCATGGA | 285 | E53SL1 | TTCAACACAATGGCTGGAA |
| | | AGG | | 8 | GCT |
| 278 | E53SL11 | GCCAAGCTTGAGTCATGGA | 286 | E53SL1 | GTTAAAGGATTCAACACAA |
| | | AGG | | 9 | TGG |
| 278 | E53SL11 | GCCAAGCTTGAGTCATGGA | 287 | E53SL2 | AAATGTTAAAGGATTCAAC |
| | | AGG | | 0 | ACA |
| 278 | E53SL11 | GCCAAGCTTGAGTCATGGA | 288 | E53SL2 | GCAACAGTTGAATGAAATG |
| | | AGG | | 1 | TTA |
| 278 | E53SL11 | GCCAAGCTTGAGTCATGGA | 289 | E53SL2 | TACAAGAACACCTTCAGAA |
| | | AGG | | 2 | CCG |
| 278 | E53SL11 | GCCAAGCTTGAGTCATGGA | 290 | E53SL2 | AAGTACAAGAACACCTTCA |
| | | AGG | | 3 | GAA |
| 278 | E53SL11 | GCCAAGCTTGAGTCATGGA | 291 | E53SL2 | AGTTGAAAGAATTCAGAAT |
| | | AGG | | 4 | CAG |
| 278 | E53SL11 | GCCAAGCTTGAGTCATGGA | 292 | E53SL2 | TAGTTGAAAGAATTCAGAA |
| | | AGG | | 5 | TCA |
| 279 | E53SL12 | AGCCAAGCTTGAGTCATGG | 280 | E53SL1 | CAGAGCCAAGCTTGAGTCA |
| | | AAG | | 3 | TGG |
| 279 | E53SL12 | AGCCAAGCTTGAGTCATGG | 281 | E53SL1 | AGGCCAGAGCCAAGCTTGA |
| | | AAG | | 4 | GTC |
| 279 | E53SL12 | AGCCAAGCTTGAGTCATGG | 282 | E53SL1 | AGAAGCTGAGCAGGTCTTA |
| | | AAG | | 5 | GGA |
| 279 | E53SL12 | AGCCAAGCTTGAGTCATGG | 283 | E53SL1 | AAGGAAGAAGCTGAGCAGG |
| | | AAG | | 6 | TCT |
| 279 | E53SL12 | AGCCAAGCTTGAGTCATGG | 284 | E53SL1 | GGAAGCTAAGGAAGAAGCT |
| | | AAG | | 7 | GAG |
| 279 | E53SL12 | AGCCAAGCTTGAGTCATGG | 285 | E53SL1 | TTCAACACAATGGCTGGAA |
| | | AAG | | 8 | GCT |
| 279 | E53SL12 | AGCCAAGCTTGAGTCATGG | 286 | E53SL1 | GTTAAAGGATTCAACACAA |
| | | AAG | | 9 | TGG |
| 279 | E53SL12 | AGCCAAGCTTGAGTCATGG | 287 | E53SL2 | AAATGTTAAAGGATTCAAC |
| | | AAG | | 0 | ACA |
| 279 | E53SL12 | AGCCAAGCTTGAGTCATGG | 288 | E53SL2 | GCAACAGTTGAATGAAATG |
| | | AAG | | 1 | TTA |
| 279 | E53SL12 | AGCCAAGCTTGAGTCATGG | 289 | E53SL2 | TACAAGAACACCTTCAGAA |
| | | AAG | | 2 | CCG |
| 279 | E53SL12 | AGCCAAGCTTGAGTCATGG | 290 | E53SL2 | AAGTACAAGAACACCTTCA |
| | | AAG | | 3 | GAA |
| 279 | E53SL12 | AGCCAAGCTTGAGTCATGG | 291 | E53SL2 | AGTTGAAAGAATTCAGAAT |
| | | AAG | | 4 | CAG |
| 279 | E53SL12 | AGCCAAGCTTGAGTCATGG | 292 | E53SL2 | TAGTTGAAAGAATTCAGAA |
| | | AAG | | 5 | TCA |
| 280 | E53SL13 | CAGAGCCAAGCTTGAGTCA | 281 | E53SL1 | AGGCCAGAGCCAAGCTTGA |
| | | TGG | | 4 | GTC |
| 280 | E53SL13 | CAGAGCCAAGCTTGAGTCA | 282 | E53SL1 | AGAAGCTGAGCAGGTCTTA |
| | | TGG | | 5 | GGA |
| 280 | E53SL13 | CAGAGCCAAGCTTGAGTCA | 283 | E53SL1 | AAGGAAGAAGCTGAGCAGG |
| | | TGG | | 6 | TCT |
| 280 | E53SL13 | CAGAGCCAAGCTTGAGTCA | 284 | E53SL1 | GGAAGCTAAGGAAGAAGCT |
| | | TGG | | 7 | GAG |
| 280 | E53SL13 | CAGAGCCAAGCTTGAGTCA | 285 | E53SL1 | TTCAACACAATGGCTGGAA |
| | | TGG | | 8 | GCT |
| 280 | E53SL13 | CAGAGCCAAGCTTGAGTCA | 286 | E53SL1 | GTTAAAGGATTCAACACAA |
| | | TGG | | 9 | TGG |
| 280 | E53SL13 | CAGAGCCAAGCTTGAGTCA | 287 | E53SL2 | AAATGTTAAAGGATTCAAC |
| | | TGG | | 0 | ACA |
| 280 | E53SL13 | CAGAGCCAAGCTTGAGTCA | 288 | E53SL2 | GCAACAGTTGAATGAAATG |
| | | TGG | | 1 | TTA |
| 280 | E53SL13 | CAGAGCCAAGCTTGAGTCA | 289 | E53SL2 | TACAAGAACACCTTCAGAA |
| | | TGG | | 2 | CCG |
| 280 | E53SL13 | CAGAGCCAAGCTTGAGTCA | 290 | E53SL2 | AAGTACAAGAACACCTTCA |
| | | TGG | | 3 | GAA |
| 280 | E53SL13 | CAGAGCCAAGCTTGAGTCA | 291 | E53SL2 | AGTTGAAAGAATTCAGAAT |
| | | TGG | | 4 | CAG |
| 280 | E53SL13 | CAGAGCCAAGCTTGAGTCA | 292 | E53SL2 | TAGTTGAAAGAATTCAGAA |
| | | TGG | | 5 | TCA |
| 281 | E53SL14 | AGGCCAGAGCCAAGCTTGA | 282 | E53SL1 | AGAAGCTGAGCAGGTCTTA |
| | | GTC | | 5 | GGA |
| 281 | E53SL14 | AGGCCAGAGCCAAGCTTGA | 283 | E53SL1 | AAGGAAGAAGCTGAGCAGG |
| | | GTC | | 6 | TCT |
| 281 | E53SL14 | AGGCCAGAGCCAAGCTTGA | 284 | E53SL1 | GGAAGCTAAGGAAGAAGCT |
| | | GTC | | 7 | GAG |
| 281 | E53SL14 | AGGCCAGAGCCAAGCTTGA | 285 | E53SL1 | TTCAACACAATGGCTGGAA |
| | | GTC | | 8 | GCT |
| 281 | E53SL14 | AGGCCAGAGCCAAGCTTGA | 286 | E53SL1 | GTTAAAGGATTCAACACAA |
| | | GTC | | 9 | TGG |
| 281 | E53SL14 | AGGCCAGAGCCAAGCTTGA | 287 | E53SL2 | AAATGTTAAAGGATTCAAC |
| | | GTC | | 0 | ACA |
| 281 | E53SL14 | AGGCCAGAGCCAAGCTTGA | 288 | E53SL2 | GCAACAGTTGAATGAAATG |
| | | GTC | | 1 | TTA |
| 281 | E53SL14 | AGGCCAGAGCCAAGCTTGA | 289 | E53SL2 | TACAAGAACACCTTCAGAA |
| | | GTC | | 2 | CCG |
| 281 | E53SL14 | AGGCCAGAGCCAAGCTTGA | 290 | E53SL2 | AAGTACAAGAACACCTTCA |
| | | GTC | | 3 | GAA |
| 281 | E53SL14 | AGGCCAGAGCCAAGCTTGA | 291 | E53SL2 | AGTTGAAAGAATTCAGAAT |
| | | GTC | | 4 | CAG |
| 281 | E53SL14 | AGGCCAGAGCCAAGCTTGA | 292 | E53SL2 | TAGTTGAAAGAATTCAGAA |
| | | GTC | | 5 | TCA |
| 282 | E53SL15 | AGAAGCTGAGCAGGTCTTA | 283 | E53SL1 | AAGGAAGAAGCTGAGCAGG |
| | | GGA | | 6 | TCT |
| 282 | E53SL15 | AGAAGCTGAGCAGGTCTTA | 284 | E53SL1 | GGAAGCTAAGGAAGAAGCT |
| | | GGA | | 7 | GAG |
| 282 | E53SL15 | AGAAGCTGAGCAGGTCTTA | 285 | E53SL1 | TTCAACACAATGGCTGGAA |
| | | GGA | | 8 | GCT |
| 282 | E53SL15 | AGAAGCTGAGCAGGTCTTA | 286 | E53SL1 | GTTAAAGGATTCAACACAA |
| | | GGA | | 9 | TGG |
| 282 | E53SL15 | AGAAGCTGAGCAGGTCTTA | 287 | E53SL2 | AAATGTTAAAGGATTCAAC |
| | | GGA | | 0 | ACA |
| 282 | E53SL15 | AGAAGCTGAGCAGGTCTTA | 288 | E53SL2 | GCAACAGTTGAATGAAATG |
| | | GGA | | 1 | TTA |
| 282 | E53SL15 | AGAAGCTGAGCAGGTCTTA | 289 | E53SL2 | TACAAGAACACCTTCAGAA |
| | | GGA | | 2 | CCG |
| 282 | E53SL15 | AGAAGCTGAGCAGGTCTTA | 290 | E53SL2 | AAGTACAAGAACACCTTCA |
| | | GGA | | 3 | GAA |
| 282 | E53SL15 | AGAAGCTGAGCAGGTCTTA | 291 | E53SL2 | AGTTGAAAGAATTCAGAAT |
| | | GGA | | 4 | CAG |
| 282 | E53SL15 | AGAAGCTGAGCAGGTCTTA | 292 | E53SL2 | TAGTTGAAAGAATTCAGAA |
| | | GGA | | 5 | TCA |
| 283 | E53SL16 | AAGGAAGAAGCTGAGCAGG | 284 | E53SL1 | GGAAGCTAAGGAAGAAGCT |
| | | TCT | | 7 | GAG |
| 283 | E53SL16 | AAGGAAGAAGCTGAGCAGG | 285 | E53SL1 | TTCAACACAATGGCTGGAA |
| | | TCT | | 8 | GCT |
| 283 | E53SL16 | AAGGAAGAAGCTGAGCAGG | 286 | E53SL1 | GTTAAAGGATTCAACACAA |
| | | TCT | | 9 | TGG |
| 283 | E53SL16 | AAGGAAGAAGCTGAGCAGG | 287 | E53SL2 | AAATGTTAAAGGATTCAAC |
| | | TCT | | 0 | ACA |
| 283 | E53SL16 | AAGGAAGAAGCTGAGCAGG | 288 | E53SL2 | GCAACAGTTGAATGAAATG |
| | | TCT | | 1 | TTA |
| 283 | E53SL16 | AAGGAAGAAGCTGAGCAGG | 289 | E53SL2 | TACAAGAACACCTTCAGAA |
| | | TCT | | 2 | CCG |
| 283 | E53SL16 | AAGGAAGAAGCTGAGCAGG | 290 | E53SL2 | AAGTACAAGAACACCTTCA |
| | | TCT | | 3 | GAA |
| 283 | E53SL16 | AAGGAAGAAGCTGAGCAGG | 291 | E53SL2 | AGTTGAAAGAATTCAGAAT |
| | | TCT | | 4 | CAG |
| 283 | E53SL16 | AAGGAAGAAGCTGAGCAGG | 292 | E53SL2 | TAGTTGAAAGAATTCAGAA |
| | | TCT | | 5 | TCA |
| 284 | E53SL17 | GGAAGCTAAGGAAGAAGCT | 285 | E53SL1 | TTCAACACAATGGCTGGAA |
| | | GAG | | 8 | GCT |
| 284 | E53SL17 | GGAAGCTAAGGAAGAAGCT | 286 | E53SL1 | GTTAAAGGATTCAACACAA |
| | | GAG | | 9 | TGG |
| 284 | E53SL17 | GGAAGCTAAGGAAGAAGCT | 287 | E53SL2 | AAATGTTAAAGGATTCAAC |
| | | GAG | | 0 | ACA |
| 284 | E53SL17 | GGAAGCTAAGGAAGAAGCT | 288 | E53SL2 | GCAACAGTTGAATGAAATG |
| | | GAG | | 1 | TTA |
| 284 | E53SL17 | GGAAGCTAAGGAAGAAGCT | 289 | E53SL2 | TACAAGAACACCTTCAGAA |
| | | GAG | | 2 | CCG |
| 284 | E53SL17 | GGAAGCTAAGGAAGAAGCT | 290 | E53SL2 | AAGTACAAGAACACCTTCA |
| | | GAG | | 3 | GAA |
| 284 | E53SL17 | GGAAGCTAAGGAAGAAGCT | 291 | E53SL2 | AGTTGAAAGAATTCAGAAT |
| | | GAG | | 4 | CAG |
| 284 | E53SL17 | GGAAGCTAAGGAAGAAGCT | 292 | E53SL2 | TAGTTGAAAGAATTCAGAA |
| | | GAG | | 5 | TCA |
| 285 | E53SL18 | TTCAACACAATGGCTGGAA | 286 | E53SL1 | GTTAAAGGATTCAACACAA |
| | | GCT | | 9 | TGG |
| 285 | E53SL18 | TTCAACACAATGGCTGGAA | 287 | E53SL2 | AAATGTTAAAGGATTCAAC |
| | | GCT | | 0 | ACA |
| 285 | E53SL18 | TTCAACACAATGGCTGGAA | 288 | E53SL2 | GCAACAGTTGAATGAAATG |
| | | GCT | | 1 | TTA |
| 285 | E53SL18 | TTCAACACAATGGCTGGAA | 289 | E53SL2 | TACAAGAACACCTTCAGAA |
| | | GCT | | 2 | CCG |
| 285 | E53SL18 | TTCAACACAATGGCTGGAA | 290 | E53SL2 | AAGTACAAGAACACCTTCA |
| | | GCT | | 3 | GAA |
| 285 | E53SL18 | TTCAACACAATGGCTGGAA | 291 | E53SL2 | AGTTGAAAGAATTCAGAAT |
| | | GCT | | 4 | CAG |
| 285 | E53SL18 | TTCAACACAATGGCTGGAA | 292 | E53SL2 | TAGTTGAAAGAATTCAGAA |
| | | GCT | | 5 | TCA |
| 286 | E53SL19 | GTTAAAGGATTCAACACAA | 287 | E53SL2 | AAATGTTAAAGGATTCAAC |
| | | TGG | | 0 | ACA |
| 286 | E53SL19 | GTTAAAGGATTCAACACAA | 288 | E53SL2 | GCAACAGTTGAATGAAATG |
| | | TGG | | 1 | TTA |
| 286 | E53SL19 | GTTAAAGGATTCAACACAA | 289 | E53SL2 | TACAAGAACACCTTCAGAA |
| | | TGG | | 2 | CCG |
| 286 | E53SL19 | GTTAAAGGATTCAACACAA | 290 | E53SL2 | AAGTACAAGAACACCTTCA |
| | | TGG | | 3 | GAA |
| 286 | E53SL19 | GTTAAAGGATTCAACACAA | 291 | E53SL2 | AGTTGAAAGAATTCAGAAT |
| | | TGG | | 4 | CAG |
| 286 | E53SL19 | GTTAAAGGATTCAACACAA | 292 | E53SL2 | TAGTTGAAAGAATTCAGAA |
| | | TGG | | 5 | TCA |
| 287 | E53SL20 | AAATGTTAAAGGATTCAAC | 288 | E53SL2 | GCAACAGTTGAATGAAATG |
| | | ACA | | 1 | TTA |
| 287 | E53SL20 | AAATGTTAAAGGATTCAAC | 289 | E53SL2 | TACAAGAACACCTTCAGAA |
| | | ACA | | 2 | CCG |
| 287 | E53SL20 | AAATGTTAAAGGATTCAAC | 290 | E53SL2 | AAGTACAAGAACACCTTCA |
| | | ACA | | 3 | GAA |
| 287 | E53SL20 | AAATGTTAAAGGATTCAAC | 291 | E53SL2 | AGTTGAAAGAATTCAGAAT |
| | | ACA | | 4 | CAG |
| 287 | E53SL20 | AAATGTTAAAGGATTCAAC | 292 | E53SL2 | TAGTTGAAAGAATTCAGAA |
| | | ACA | | 5 | TCA |
| 288 | E53SL21 | GCAACAGTTGAATGAAATG | 289 | E53SL2 | TACAAGAACACCTTCAGAA |
| | | TTA | | 2 | CCG |
| 288 | E53SL21 | GCAACAGTTGAATGAAATG | 290 | E53SL2 | AAGTACAAGAACACCTTCA |
| | | TTA | | 3 | GAA |
| 288 | E53SL21 | GCAACAGTTGAATGAAATG | 291 | E53SL2 | AGTTGAAAGAATTCAGAAT |
| | | TTA | | 4 | CAG |
| 288 | E53SL21 | GCAACAGTTGAATGAAATG | 292 | E53SL2 | TAGTTGAAAGAATTCAGAA |
| | | TTA | | 5 | TCA |
| 289 | E53SL22 | TACAAGAACACCTTCAGAA | 290 | E53SL2 | AAGTACAAGAACACCTTCA |
| | | CCG | | 3 | GAA |
| 289 | E53SL22 | TACAAGAACACCTTCAGAA | 291 | E53SL2 | AGTTGAAAGAATTCAGAAT |
| | | CCG | | 4 | CAG |
| 289 | E53SL22 | TACAAGAACACCTTCAGAA | 292 | E53SL2 | TAGTTGAAAGAATTCAGAA |
| | | CCG | | 5 | TCA |
| 290 | E53SL23 | AAGTACAAGAACACCTTCA | 291 | E53SL2 | AGTTGAAAGAATTCAGAAT |
| | | GAA | | 4 | CAG |
| 290 | E53SL23 | AAGTACAAGAACACCTTCA | 292 | E53SL2 | TAGTTGAAAGAATTCAGAA |
| | | GAA | | 5 | TCA |
| 291 | E53SL24 | AGTTGAAAGAATTCAGAAT | 292 | E53SL2 | TAGTTGAAAGAATTCAGAA |
| | | CAG | | 5 | TCA |
| 924 | E53SL3.1A-Cv5 | CATTTGGATTGCATCTACT | 272 | E53SL4 | TTTTGGATTGCATCTACTG |
| | | GTA | | | TAT |
| 924 | E53SL3.1A-Cv5 | CATTTGGATTGCATCTACT | 273 | E53SL5 | ACCCTCCTTCCATGACTCA |
| | | GTA | | | AGC |
| 924 | E53SL3.1A-Cv5 | CATTTGGATTGCATCTACT | 274 | E53SL6 | CTTCCATGACTCAAGCTTG |
| | | GTA | | | GCT |
| 924 | E53SL3.1A-Cv5 | CATTTGGATTGCATCTACT | 275 | E53SL7 | ACATTTCATTCAACTGTTG |
| | | GTA | | | CCT |
| 924 | E53SL3.1A-Cv5 | CATTTGGATTGCATCTACT | 276 | E53SL8 | TCAACTGTTGCCTCCGGTT |
| | | GTA | | | CTG |
| 924 | E53SL3.1A-Cv5 | CATTTGGATTGCATCTACT | 277 | E53SL1 | CCAAAAGAAAATCACAGAA |
| | | GTA | | 0 | ACC |
| 924 | E53SL3.1A-Cv5 | CATTTGGATTGCATCTACT | 278 | E53SL1 | GCCAAGCTTGAGTCATGGA |
| | | GTA | | 1 | AGG |
| 924 | E53SL3.1A-Cv5 | CATTTGGATTGCATCTACT | 279 | E53SL1 | AGCCAAGCTTGAGTCATGG |
| | | GTA | | 2 | AAG |
| 924 | E53SL3.1A-Cv5 | CATTTGGATTGCATCTACT | 280 | E53SL1 | CAGAGCCAAGCTTGAGTCA |
| | | GTA | | 3 | TGG |
| 924 | E53SL3.1A-Cv5 | CATTTGGATTGCATCTACT | 281 | E53SL1 | AGGCCAGAGCCAAGCTTGA |
| | | GTA | | 4 | GTC |
| 924 | E53SL3.1A-Cv5 | CATTTGGATTGCATCTACT | 282 | E53SL1 | AGAAGCTGAGCAGGTCTTA |
| | | GTA | | 5 | GGA |
| 924 | E53SL3.1A-Cv5 | CATTTGGATTGCATCTACT | 283 | E53SL1 | AAGGAAGAAGCTGAGCAGG |
| | | GTA | | 6 | TCT |
| 924 | E53SL3.1A-Cv5 | CATTTGGATTGCATCTACT | 284 | E53SL1 | GGAAGCTAAGGAAGAAGCT |
| | | GTA | | 7 | GAG |
| 924 | E53SL3.1A-Cv5 | CATTTGGATTGCATCTACT | 285 | E53SL1 | TTCAACACAATGGCTGGAA |
| | | GTA | | 8 | GCT |
| 924 | E53SL3.1A-Cv5 | CATTTGGATTGCATCTACT | 286 | E53SL1 | GTTAAAGGATTCAACACAA |
| | | GTA | | 9 | TGG |
| 924 | E53SL3.1A-Cv5 | CATTTGGATTGCATCTACT | 287 | E53SL2 | AAATGTTAAAGGATTCAAC |
| | | GTA | | 0 | ACA |
| 924 | E53SL3.1A-Cv5 | CATTTGGATTGCATCTACT | 288 | E53SL2 | GCAACAGTTGAATGAAATG |
| | | GTA | | 1 | TTA |
| 924 | E53SL3.1A-Cv5 | CATTTGGATTGCATCTACT | 289 | E53SL2 | TACAAGAACACCTTCAGAA |
| | | GTA | | 2 | CCG |
| 924 | E53SL3.1A-Cv5 | CATTTGGATTGCATCTACT | 290 | E53SL2 | AAGTACAAGAACACCTTCA |
| | | GTA | | 3 | GAA |
| 924 | E53SL3.1A-Cv5 | CATTTGGATTGCATCTACT | 291 | E53SL2 | AGTTGAAAGAATTCAGAAT |
| | | GTA | | 4 | CAG |
| 924 | E53SL3.1A-Cv5 | CATTTGGATTGCATCTACT | 292 | E53SL2 | TAGTTGAAAGAATTCAGAA |
| | | GTA | | 5 | TCA |
| 925 | E53SL3.1C-Cv5 | CCTTTGGATTGCATCTACT | 272 | E53SL4 | TTTTGGATTGCATCTACTG |
| | | GTA | | | TAT |
| 925 | E53SL3.1C-Cv5 | CCTTTGGATTGCATCTACT | 273 | E53SL5 | ACCCTCCTTCCATGACTCA |
| | | GTA | | | AGC |
| 925 | E53SL3.1C-Cv5 | CCTTTGGATTGCATCTACT | 274 | E53SL6 | CTTCCATGACTCAAGCTTG |
| | | GTA | | | GCT |
| 925 | E53SL3.1C-Cv5 | CCTTTGGATTGCATCTACT | 275 | E53SL7 | ACATTTCATTCAACTGTTG |
| | | GTA | | | CCT |
| 925 | E53SL3.1C-Cv5 | CCTTTGGATTGCATCTACT | 276 | E53SL8 | TCAACTGTTGCCTCCGGTT |
| | | GTA | | | CTG |
| 925 | E53SL3.1C-Cv5 | CCTTTGGATTGCATCTACT | 277 | E53SL1 | CCAAAAGAAAATCACAGAA |
| | | GTA | | 0 | ACC |
| 925 | E53SL3.1C-Cv5 | CCTTTGGATTGCATCTACT | 278 | E53SL1 | GCCAAGCTTGAGTCATGGA |
| | | GTA | | 1 | AGG |
| 925 | E53SL3.1C-Cv5 | CCTTTGGATTGCATCTACT | 279 | E53SL1 | AGCCAAGCTTGAGTCATGG |
| | | GTA | | 2 | AAG |
| 925 | E53SL3.1C-Cv5 | CCTTTGGATTGCATCTACT | 280 | E53SL1 | CAGAGCCAAGCTTGAGTCA |
| | | GTA | | 3 | TGG |
| 925 | E53SL3.1C-Cv5 | CCTTTGGATTGCATCTACT | 281 | E53SL1 | AGGCCAGAGCCAAGCTTGA |
| | | GTA | | 4 | GTC |
| 925 | E53SL3.1C-Cv5 | CCTTTGGATTGCATCTACT | 282 | E53SL1 | AGAAGCTGAGCAGGTCTTA |
| | | GTA | | 5 | GGA |
| 925 | E53SL3.1C-Cv5 | CCTTTGGATTGCATCTACT | 283 | E53SL1 | AAGGAAGAAGCTGAGCAGG |
| | | GTA | | 6 | TCT |
| 925 | E53SL3.1C-Cv5 | CCTTTGGATTGCATCTACT | 284 | E53SL1 | GGAAGCTAAGGAAGAAGCT |
| | | GTA | | 7 | GAG |
| 925 | E53SL3.1C-Cv5 | CCTTTGGATTGCATCTACT | 285 | E53SL1 | TTCAACACAATGGCTGGAA |
| | | GTA | | 8 | GCT |
| 925 | E53SL3.1C-Cv5 | CCTTTGGATTGCATCTACT | 286 | E53SL1 | GTTAAAGGATTCAACACAA |
| | | GTA | | 9 | TGG |
| 925 | E53SL3.1C-Cv5 | CCTTTGGATTGCATCTACT | 287 | E53SL2 | AAATGTTAAAGGATTCAAC |
| | | GTA | | 0 | ACA |
| 925 | E53SL3.1C-Cv5 | CCTTTGGATTGCATCTACT | 288 | E53SL2 | GCAACAGTTGAATGAAATG |
| | | GTA | | 1 | TTA |
| 925 | E53SL3.1C-Cv5 | CCTTTGGATTGCATCTACT | 289 | E53SL2 | TACAAGAACACCTTCAGAA |
| | | GTA | | 2 | CCG |
| 925 | E53SL3.1C-Cv5 | CCTTTGGATTGCATCTACT | 290 | E53SL2 | AAGTACAAGAACACCTTCA |
| | | GTA | | 3 | GAA |
| 925 | E53SL3.1C-Cv5 | CCTTTGGATTGCATCTACT | 291 | E53SL2 | AGTTGAAAGAATTCAGAAT |
| | | GTA | | 4 | CAG |
| 925 | E53SL3.1C-Cv5 | CCTTTGGATTGCATCTACT | 292 | E53SL2 | TAGTTGAAAGAATTCAGAA |
| | | GTA | | 5 | TCA |
| 926 | E53S13.1G-Cv5 | CGTTTGGATTGCATCTACT | 272 | E53SL4 | TTTTGGATTGCATCTACTG |
| | | GTA | | | TAT |
| 926 | E53S13.1G-Cv5 | CGTTTGGATTGCATCTACT | 273 | E53SL5 | ACCCTCCTTCCATGACTCA |
| | | GTA | | | AGC |
| 926 | E53S13.1G-Cv5 | CGTTTGGATTGCATCTACT | 274 | E53SL6 | CTTCCATGACTCAAGCTTG |
| | | GTA | | | GCT |
| 926 | E53S13.1G-Cv5 | CGTTTGGATTGCATCTACT | 275 | E53SL7 | ACATTTCATTCAACTGTTG |
| | | GTA | | | CCT |
| 926 | E53S13.1G-Cv5 | CGTTTGGATTGCATCTACT | 276 | E53SL8 | TCAACTGTTGCCTCCGGTT |
| | | GTA | | | CTG |
| 926 | E53S13.1G-Cv5 | CGTTTGGATTGCATCTACT | 277 | E53SL1 | CCAAAAGAAAATCACAGAA |
| | | GTA | | 0 | ACC |
| 926 | E53S13.1G-Cv5 | CGTTTGGATTGCATCTACT | 278 | E53SL1 | GCCAAGCTTGAGTCATGGA |
| | | GTA | | 1 | AGG |
| 926 | E53S13.1G-Cv5 | CGTTTGGATTGCATCTACT | 279 | E53SL1 | AGCCAAGCTTGAGTCATGG |
| | | GTA | | 2 | AAG |
| 926 | E53S13.1G-Cv5 | CGTTTGGATTGCATCTACT | 280 | E53SL1 | CAGAGCCAAGCTTGAGTCA |
| | | GTA | | 3 | TGG |
| 926 | E53S13.1G-Cv5 | CGTTTGGATTGCATCTACT | 281 | E53SL1 | AGGCCAGAGCCAAGCTTGA |
| | | GTA | | 4 | GTC |
| 926 | E53S13.1G-Cv5 | CGTTTGGATTGCATCTACT | 282 | E53SL1 | AGAAGCTGAGCAGGTCTTA |
| | | GTA | | 5 | GGA |
| 926 | E53S13.1G-Cv5 | CGTTTGGATTGCATCTACT | 283 | E53SL1 | AAGGAAGAAGCTGAGCAGG |
| | | GTA | | 6 | TCT |
| 926 | E53S13.1G-Cv5 | CGTTTGGATTGCATCTACT | 284 | E53SL1 | GGAAGCTAAGGAAGAAGCT |
| | | GTA | | 7 | GAG |
| 926 | E53S13.1G-Cv5 | CGTTTGGATTGCATCTACT | 285 | E53SL1 | TTCAACACAATGGCTGGAA |
| | | GTA | | 8 | GCT |
| 926 | E53S13.1G-Cv5 | CGTTTGGATTGCATCTACT | 286 | E53SL1 | GTTAAAGGATTCAACACAA |
| | | GTA | | 9 | TGG |
| 926 | E53S13.1G-Cv5 | CGTTTGGATTGCATCTACT | 287 | E53SL2 | AAATGTTAAAGGATTCAAC |
| | | GTA | | 0 | ACA |
| 926 | E53S13.1G-Cv5 | CGTTTGGATTGCATCTACT | 288 | E53SL2 | GCAACAGTTGAATGAAATG |
| | | GTA | | 1 | TTA |
| 926 | E53S13.1G-Cv5 | CGTTTGGATTGCATCTACT | 289 | E53SL2 | TACAAGAACACCTTCAGAA |
| | | GTA | | 2 | CCG |
| 926 | E53S13.1G-Cv5 | CGTTTGGATTGCATCTACT | 290 | E53SL2 | AAGTACAAGAACACCTTCA |
| | | GTA | | 3 | GAA |
| 926 | E53S13.1G-Cv5 | CGTTTGGATTGCATCTACT | 291 | E53SL2 | AGTTGAAAGAATTCAGAAT |
| | | GTA | | 4 | CAG |
| 926 | E53S13.1G-Cv5 | CGTTTGGATTGCATCTACT | 292 | E53SL2 | TAGTTGAAAGAATTCAGAA |
| | | GTA | | 5 | TCA |
| 927 | E53S13.2A-Cv5 | CTATTGGATTGCATCTACT | 272 | E53SL4 | TTTTGGATTGCATCTACTG |
| | | GTA | | | TAT |
| 927 | E53S13.2A-Cv5 | CTATTGGATTGCATCTACT | 273 | E53SL5 | ACCCTCCTTCCATGACTCA |
| | | GTA | | | AGC |
| 927 | E53S13.2A-Cv5 | CTATTGGATTGCATCTACT | 274 | E53SL6 | CTTCCATGACTCAAGCTTG |
| | | GTA | | | GCT |
| 927 | E53S13.2A-Cv5 | CTATTGGATTGCATCTACT | 275 | E53SL7 | ACATTTCATTCAACTGTTG |
| | | GTA | | | CCT |
| 927 | E53S13.2A-Cv5 | CTATTGGATTGCATCTACT | 276 | E53SL8 | TCAACTGTTGCCTCCGGTT |
| | | GTA | | | CTG |
| 927 | E53S13.2A-Cv5 | CTATTGGATTGCATCTACT | 277 | E53SL1 | CCAAAAGAAAATCACAGAA |
| | | GTA | | 0 | ACC |
| 927 | E53S13.2A-Cv5 | CTATTGGATTGCATCTACT | 278 | E53SL1 | GCCAAGCTTGAGTCATGGA |
| | | GTA | | 1 | AGG |
| 927 | E53S13.2A-Cv5 | CTATTGGATTGCATCTACT | 279 | E53SL1 | AGCCAAGCTTGAGTCATGG |
| | | GTA | | 2 | AAG |
| 927 | E53S13.2A-Cv5 | CTATTGGATTGCATCTACT | 280 | E53SL1 | CAGAGCCAAGCTTGAGTCA |
| | | GTA | | 3 | TGG |
| 927 | E53S13.2A-Cv5 | CTATTGGATTGCATCTACT | 281 | E53SL1 | AGGCCAGAGCCAAGCTTGA |
| | | GTA | | 4 | GTC |
| 927 | E53S13.2A-Cv5 | CTATTGGATTGCATCTACT | 282 | E53SL1 | AGAAGCTGAGCAGGTCTTA |
| | | GTA | | 5 | GGA |
| 927 | E53S13.2A-Cv5 | CTATTGGATTGCATCTACT | 283 | E53SL1 | AAGGAAGAAGCTGAGCAGG |
| | | GTA | | 6 | TCT |
| 927 | E53S13.2A-Cv5 | CTATTGGATTGCATCTACT | 284 | E53SL1 | GGAAGCTAAGGAAGAAGCT |
| | | GTA | | 7 | GAG |
| 927 | E53S13.2A-Cv5 | CTATTGGATTGCATCTACT | 285 | E53SL1 | TTCAACACAATGGCTGGAA |
| | | GTA | | 8 | GCT |
| 927 | E53S13.2A-Cv5 | CTATTGGATTGCATCTACT | 286 | E53SL1 | GTTAAAGGATTCAACACAA |
| | | GTA | | 9 | TGG |
| 927 | E53S13.2A-Cv5 | CTATTGGATTGCATCTACT | 287 | E53SL2 | AAATGTTAAAGGATTCAAC |
| | | GTA | | 0 | ACA |
| 927 | E53S13.2A-Cv5 | CTATTGGATTGCATCTACT | 288 | E53SL2 | GCAACAGTTGAATGAAATG |
| | | GTA | | 1 | TTA |
| 927 | E53S13.2A-Cv5 | CTATTGGATTGCATCTACT | 289 | E53SL2 | TACAAGAACACCTTCAGAA |
| | | GTA | | 2 | CCG |
| 927 | E53S13.2A-Cv5 | CTATTGGATTGCATCTACT | 290 | E53SL2 | AAGTACAAGAACACCTTCA |
| | | GTA | | 3 | GAA |
| 927 | E53S13.2A-Cv5 | CTATTGGATTGCATCTACT | 291 | E53SL2 | AGTTGAAAGAATTCAGAAT |
| | | GTA | | 4 | CAG |
| 927 | E53S13.2A-Cv5 | CTATTGGATTGCATCTACT | 292 | E53SL2 | TAGTTGAAAGAATTCAGAA |
| | | GTA | | 5 | TCA |
| 928 | E53S13.2C-Cv5 | CTCTTGGATTGCATCTACT | 272 | E53SL4 | TTTTGGATTGCATCTACTG |
| | | GTA | | | TAT |
| 928 | E53S13.2C-Cv5 | CTCTTGGATTGCATCTACT | 273 | E53SL5 | ACCCTCCTTCCATGACTCA |
| | | GTA | | | AGC |
| 928 | E53S13.2C-Cv5 | CTCTTGGATTGCATCTACT | 274 | E53SL6 | CTTCCATGACTCAAGCTTG |
| | | GTA | | | GCT |
| 928 | E53S13.2C-Cv5 | CTCTTGGATTGCATCTACT | 275 | E53SL7 | ACATTTCATTCAACTGTTG |
| | | GTA | | | CCT |
| 928 | E53S13.2C-Cv5 | CTCTTGGATTGCATCTACT | 276 | E53SL8 | TCAACTGTTGCCTCCGGTT |
| | | GTA | | | CTG |
| 928 | E53S13.2C-Cv5 | CTCTTGGATTGCATCTACT | 277 | E53SL1 | CCAAAAGAAAATCACAGAA |
| | | GTA | | 0 | ACC |
| 928 | E53S13.2C-Cv5 | CTCTTGGATTGCATCTACT | 278 | E53SL1 | GCCAAGCTTGAGTCATGGA |
| | | GTA | | 1 | AGG |
| 928 | E53S13.2C-Cv5 | CTCTTGGATTGCATCTACT | 279 | E53SL1 | AGCCAAGCTTGAGTCATGG |
| | | GTA | | 2 | AAG |
| 928 | E53S13.2C-Cv5 | CTCTTGGATTGCATCTACT | 280 | E53SL1 | CAGAGCCAAGCTTGAGTCA |
| | | GTA | | 3 | TGG |
| 928 | E53S13.2C-Cv5 | CTCTTGGATTGCATCTACT | 281 | E53SL1 | AGGCCAGAGCCAAGCTTGA |
| | | GTA | | 4 | GTC |
| 928 | E53S13.2C-Cv5 | CTCTTGGATTGCATCTACT | 282 | E53SL1 | AGAAGCTGAGCAGGTCTTA |
| | | GTA | | 5 | GGA |
| 928 | E53S13.2C-Cv5 | CTCTTGGATTGCATCTACT | 283 | E53SL1 | AAGGAAGAAGCTGAGCAGG |
| | | GTA | | 6 | TCT |
| 928 | E53S13.2C-Cv5 | CTCTTGGATTGCATCTACT | 284 | E53SL1 | GGAAGCTAAGGAAGAAGCT |
| | | GTA | | 7 | GAG |
| 928 | E53S13.2C-Cv5 | CTCTTGGATTGCATCTACT | 285 | E53SL1 | TTCAACACAATGGCTGGAA |
| | | GTA | | 8 | GCT |
| 928 | E53S13.2C-Cv5 | CTCTTGGATTGCATCTACT | 286 | E53SL1 | GTTAAAGGATTCAACACAA |
| | | GTA | | 9 | TGG |
| 928 | E53S13.2C-Cv5 | CTCTTGGATTGCATCTACT | 287 | E53SL2 | AAATGTTAAAGGATTCAAC |
| | | GTA | | 0 | ACA |
| 928 | E53S13.2C-Cv5 | CTCTTGGATTGCATCTACT | 288 | E53SL2 | GCAACAGTTGAATGAAATG |
| | | GTA | | 1 | TTA |
| 928 | E53S13.2C-Cv5 | CTCTTGGATTGCATCTACT | 289 | E53SL2 | TACAAGAACACCTTCAGAA |
| | | GTA | | 2 | CCG |
| 928 | E53S13.2C-Cv5 | CTCTTGGATTGCATCTACT | 290 | E53SL2 | AAGTACAAGAACACCTTCA |
| | | GTA | | 3 | GAA |
| 928 | E53S13.2C-Cv5 | CTCTTGGATTGCATCTACT | 291 | E53SL2 | AGTTGAAAGAATTCAGAAT |
| | | GTA | | 4 | CAG |
| 928 | E53S13.2C-Cv5 | CTCTTGGATTGCATCTACT | 292 | E53SL2 | TAGTTGAAAGAATTCAGAA |
| | | GTA | | 5 | TCA |
| 929 | E53S13.2G-Cv5 | CTGTTGGATTGCATCTACT | 272 | E53SL4 | TTTTGGATTGCATCTACTG |
| | | GTA | | | TAT |
| 929 | E53S13.2G-Cv5 | CTGTTGGATTGCATCTACT | 273 | E53SL5 | ACCCTCCTTCCATGACTCA |
| | | GTA | | | AGC |
| 929 | E53S13.2G-Cv5 | CTGTTGGATTGCATCTACT | 274 | E53SL6 | CTTCCATGACTCAAGCTTG |
| | | GTA | | | GCT |
| 929 | E53S13.2G-Cv5 | CTGTTGGATTGCATCTACT | 275 | E53SL7 | ACATTTCATTCAACTGTTG |
| | | GTA | | | CCT |
| 929 | E53S13.2G-Cv5 | CTGTTGGATTGCATCTACT | 276 | E53SL8 | TCAACTGTTGCCTCCGGTT |
| | | GTA | | | CTG |
| 929 | E53S13.2G-Cv5 | CTGTTGGATTGCATCTACT | 277 | E53SL1 | CCAAAAGAAAATCACAGAA |
| | | GTA | | 0 | ACC |
| 929 | E53S13.2G-Cv5 | CTGTTGGATTGCATCTACT | 278 | E53SL1 | GCCAAGCTTGAGTCATGGA |
| | | GTA | | 1 | AGG |
| 929 | E53S13.2G-Cv5 | CTGTTGGATTGCATCTACT | 279 | E53SL1 | AGCCAAGCTTGAGTCATGG |
| | | GTA | | 2 | AAG |
| 929 | E53S13.2G-Cv5 | CTGTTGGATTGCATCTACT | 280 | E53SL1 | CAGAGCCAAGCTTGAGTCA |
| | | GTA | | 3 | TGG |
| 929 | E53S13.2G-Cv5 | CTGTTGGATTGCATCTACT | 281 | E53SL1 | AGGCCAGAGCCAAGCTTGA |
| | | GTA | | 4 | GTC |
| 929 | E53S13.2G-Cv5 | CTGTTGGATTGCATCTACT | 282 | E53SL1 | AGAAGCTGAGCAGGTCTTA |
| | | GTA | | 5 | GGA |
| 929 | E53S13.2G-Cv5 | CTGTTGGATTGCATCTACT | 283 | E53SL1 | AAGGAAGAAGCTGAGCAGG |
| | | GTA | | 6 | TCT |
| 929 | E53S13.2G-Cv5 | CTGTTGGATTGCATCTACT | 284 | E53SL1 | GGAAGCTAAGGAAGAAGCT |
| | | GTA | | 7 | GAG |
| 929 | E53S13.2G-Cv5 | CTGTTGGATTGCATCTACT | 285 | E53SL1 | TTCAACACAATGGCTGGAA |
| | | GTA | | 8 | GCT |
| 929 | E53S13.2G-Cv5 | CTGTTGGATTGCATCTACT | 286 | E53SL1 | GTTAAAGGATTCAACACAA |
| | | GTA | | 9 | TGG |
| 929 | E53S13.2G-Cv5 | CTGTTGGATTGCATCTACT | 287 | E53SL2 | AAATGTTAAAGGATTCAAC |
| | | GTA | | 0 | ACA |
| 929 | E53S13.2G-Cv5 | CTGTTGGATTGCATCTACT | 288 | E53SL2 | GCAACAGTTGAATGAAATG |
| | | GTA | | 1 | TTA |
| 929 | E53S13.2G-Cv5 | CTGTTGGATTGCATCTACT | 289 | E53SL2 | TACAAGAACACCTTCAGAA |
| | | GTA | | 2 | CCG |
| 929 | E53S13.2G-Cv5 | CTGTTGGATTGCATCTACT | 290 | E53SL2 | AAGTACAAGAACACCTTCA |
| | | GTA | | 3 | GAA |
| 929 | E53S13.2G-Cv5 | CTGTTGGATTGCATCTACT | 291 | E53SL2 | AGTTGAAAGAATTCAGAAT |
| | | GTA | | 4 | CAG |
| 929 | E53S13.2G-Cv5 | CTGTTGGATTGCATCTACT | 292 | E53SL2 | TAGTTGAAAGAATTCAGAA |
| | | GTA | | 5 | TCA |
| 930 | E53S13.3A-Cv5 | CTTATGGATTGCATCTACT | 272 | E53SL4 | TTTTGGATTGCATCTACTG |
| | | GTA | | | TAT |
| 930 | E53S13.3A-Cv5 | CTTATGGATTGCATCTACT | 273 | E53SL5 | ACCCTCCTTCCATGACTCA |
| | | GTA | | | AGC |
| 930 | E53S13.3A-Cv5 | CTTATGGATTGCATCTACT | 274 | E53SL6 | CTTCCATGACTCAAGCTTG |
| | | GTA | | | GCT |
| 930 | E53S13.3A-Cv5 | CTTATGGATTGCATCTACT | 275 | E53SL7 | ACATTTCATTCAACTGTTG |
| | | GTA | | | CCT |
| 930 | E53S13.3A-Cv5 | CTTATGGATTGCATCTACT | 276 | | TCAACTGTTGCCTCCGGTT |
| | | GTA | | E53SL8 | CTG |
| 930 | E53S13.3A-Cv5 | CTTATGGATTGCATCTACT | 277 | E53SL1 | CCAAAAGAAAATCACAGAA |
| | | GTA | | 0 | ACC |
| 930 | E53S13.3A-Cv5 | CTTATGGATTGCATCTACT | 278 | E53SL1 | GCCAAGCTTGAGTCATGGA |
| | | GTA | | 1 | AGG |
| 930 | E53S13.3A-Cv5 | CTTATGGATTGCATCTACT | 279 | E53SL1 | AGCCAAGCTTGAGTCATGG |
| | | GTA | | 2 | AAG |
| 930 | E53S13.3A-Cv5 | CTTATGGATTGCATCTACT | 280 | E53SL1 | CAGAGCCAAGCTTGAGTCA |
| | | GTA | | 3 | TGG |
| 930 | E53S13.3A-Cv5 | CTTATGGATTGCATCTACT | 281 | E53SL1 | AGGCCAGAGCCAAGCTTGA |
| | | GTA | | 4 | GTC |
| 930 | E53S13.3A-Cv5 | CTTATGGATTGCATCTACT | 282 | E53SL1 | AGAAGCTGAGCAGGTCTTA |
| | | GTA | | 5 | GGA |
| 930 | E53S13.3A-Cv5 | CTTATGGATTGCATCTACT | 283 | E53SL1 | AAGGAAGAAGCTGAGCAGG |
| | | GTA | | 6 | TCT |
| 930 | E53S13.3A-Cv5 | CTTATGGATTGCATCTACT | 284 | E53SL1 | GGAAGCTAAGGAAGAAGCT |
| | | GTA | | 7 | GAG |
| 930 | E53S13.3A-Cv5 | CTTATGGATTGCATCTACT | 285 | E53SL1 | TTCAACACAATGGCTGGAA |
| | | GTA | | 8 | GCT |
| 930 | E53S13.3A-Cv5 | CTTATGGATTGCATCTACT | 286 | E53SL1 | GTTAAAGGATTCAACACAA |
| | | GTA | | 9 | TGG |
| 930 | E53S13.3A-Cv5 | CTTATGGATTGCATCTACT | 287 | E53SL2 | AAATGTTAAAGGATTCAAC |
| | | GTA | | 0 | ACA |
| 930 | E53S13.3A-Cv5 | CTTATGGATTGCATCTACT | 288 | E53SL2 | GCAACAGTTGAATGAAATG |
| | | GTA | | 7 | TTA |
| 930 | E53S13.3A-Cv5 | CTTATGGATTGCATCTACT | 289 | E53SL2 | TACAAGAACACCTTCAGAA |
| | | GTA | | 2 | CCG |
| 930 | E53S13.3A-Cv5 | CTTATGGATTGCATCTACT | 290 | E53SL2 | AAGTACAAGAACACCTTCA |
| | | GTA | | 3 | GAA |
| 930 | E53S13.3A-Cv5 | CTTATGGATTGCATCTACT | 291 | E53SL2 | AGTTGAAAGAATTCAGAAT |
| | | GTA | | 4 | CAG |
| 930 | E53S13.3A-Cv5 | CTTATGGATTGCATCTACT | 292 | E53SL2 | TAGTTGAAAGAATTCAGAA |
| | | GTA | | 5 | TCA |
| 931 | E53S13.3C-Cv5 | CTTCTGGATTGCATCTACT | 272 | E53SL4 | TTTTGGATTGCATCTACTG |
| | | GTA | | | TAT |
| 931 | E53S13.3C-Cv5 | CTTCTGGATTGCATCTACT | 273 | E53SL5 | ACCCTCCTTCCATGACTCA |
| | | GTA | | | AGC |
| 931 | E53S13.3C-Cv5 | CTTCTGGATTGCATCTACT | 274 | E53SL6 | CTTCCATGACTCAAGCTTG |
| | | GTA | | | GCT |
| 931 | E53S13.3C-Cv5 | CTTCTGGATTGCATCTACT | 275 | E53SL7 | ACATTTCATTCAACTGTTG |
| | | GTA | | | CCT |
| 931 | E53S13.3C-Cv5 | CTTCTGGATTGCATCTACT | 276 | E53SL8 | TCAACTGTTGCCTCCGGTT |
| | | GTA | | | CTG |
| 931 | E53S13.3C-Cv5 | CTTCTGGATTGCATCTACT | 277 | E53SL1 | CCAAAAGAAAATCACAGAA |
| | | GTA | | 0 | ACC |
| 931 | E53S13.3C-Cv5 | CTTCTGGATTGCATCTACT | 278 | E53SL1 | GCCAAGCTTGAGTCATGGA |
| | | GTA | | 1 | AGG |
| 931 | E53S13.3C-Cv5 | CTTCTGGATTGCATCTACT | 279 | E53SL1 | AGCCAAGCTTGAGTCATGG |
| | | GTA | | 2 | AAG |
| 931 | E53S13.3C-Cv5 | CTTCTGGATTGCATCTACT | 280 | E53SL1 | CAGAGCCAAGCTTGAGTCA |
| | | GTA | | 3 | TGG |
| 931 | E53S13.3C-Cv5 | CTTCTGGATTGCATCTACT | 281 | E53SL1 | AGGCCAGAGCCAAGCTTGA |
| | | GTA | | 4 | GTC |
| 931 | E53S13.3C-Cv5 | CTTCTGGATTGCATCTACT | 282 | E53SL1 | AGAAGCTGAGCAGGTCTTA |
| | | GTA | | 5 | GGA |
| 931 | E53S13.3C-Cv5 | CTTCTGGATTGCATCTACT | 283 | E53SL1 | AAGGAAGAAGCTGAGCAGG |
| | | GTA | | 6 | TCT |
| 931 | E53S13.3C-Cv5 | CTTCTGGATTGCATCTACT | 284 | E53SL1 | GGAAGCTAAGGAAGAAGCT |
| | | GTA | | 7 | GAG |
| 931 | E53S13.3C-Cv5 | CTTCTGGATTGCATCTACT | 285 | E53SL1 | TTCAACACAATGGCTGGAA |
| | | GTA | | 8 | GCT |
| 931 | E53S13.3C-Cv5 | CTTCTGGATTGCATCTACT | 286 | E53SL1 | GTTAAAGGATTCAACACAA |
| | | GTA | | 9 | TGG |
| 931 | E53S13.3C-Cv5 | CTTCTGGATTGCATCTACT | 287 | E53SL2 | AAATGTTAAAGGATTCAAC |
| | | GTA | | 0 | ACA |
| 931 | E53S13.3C-Cv5 | CTTCTGGATTGCATCTACT | 288 | E53SL2 | GCAACAGTTGAATGAAATG |
| | | GTA | | 1 | TTA |
| 931 | E53S13.3C-Cv5 | CTTCTGGATTGCATCTACT | 289 | E53SL2 | TACAAGAACACCTTCAGAA |
| | | GTA | | 2 | CCG |
| 931 | E53S13.3C-Cv5 | CTTCTGGATTGCATCTACT | 290 | E53SL2 | AAGTACAAGAACACCTTCA |
| | | GTA | | 3 | GAA |
| 931 | E53S13.3C-Cv5 | CTTCTGGATTGCATCTACT | 291 | E53SL2 | AGTTGAAAGAATTCAGAAT |
| | | GTA | | 4 | CAG |
| 931 | E53S13.3C-Cv5 | CTTCTGGATTGCATCTACT | 292 | E53SL2 | TAGTTGAAAGAATTCAGAA |
| | | GTA | | 5 | TCA |
| 932 | E53S13.3G-Cv5 | CTTGTGGATTGCATCTACT | 272 | E53SL4 | TTTTGGATTGCATCTACTG |
| | | GTA | | | TAT |
| 932 | E53S13.3G-Cv5 | CTTGTGGATTGCATCTACT | 273 | E53SL5 | ACCCTCCTTCCATGACTCA |
| | | GTA | | | AGC |
| 932 | E53S13.3G-Cv5 | CTTGTGGATTGCATCTACT | 274 | E53SL6 | CTTCCATGACTCAAGCTTG |
| | | GTA | | | GCT |
| 932 | E53S13.3G-Cv5 | CTTGTGGATTGCATCTACT | 275 | E53SL7 | ACATTTCATTCAACTGTTG |
| | | GTA | | | CCT |
| 932 | E53S13.3G-Cv5 | CTTGTGGATTGCATCTACT | 276 | E53SL8 | TCAACTGTTGCCTCCGGTT |
| | | GTA | | | CTG |
| 932 | E53S13.3G-Cv5 | CTTGTGGATTGCATCTACT | 277 | E53SL1 | CCAAAAGAAAATCACAGAA |
| | | GTA | | 0 | ACC |
| 932 | E53S13.3G-Cv5 | CTTGTGGATTGCATCTACT | 278 | E53SL1 | GCCAAGCTTGAGTCATGGA |
| | | GTA | | 1 | AGG |
| 932 | E53S13.3G-Cv5 | CTTGTGGATTGCATCTACT | 279 | E53SL1 | AGCCAAGCTTGAGTCATGG |
| | | GTA | | 2 | AAG |
| 932 | E53S13.3G-Cv5 | CTTGTGGATTGCATCTACT | 280 | E53SL1 | CAGAGCCAAGCTTGAGTCA |
| | | GTA | | 3 | TGG |
| 932 | E53S13.3G-Cv5 | CTTGTGGATTGCATCTACT | 281 | E53SL1 | AGGCCAGAGCCAAGCTTGA |
| | | GTA | | 4 | GTC |
| 932 | E53S13.3G-Cv5 | CTTGTGGATTGCATCTACT | 282 | E53SL1 | AGAAGCTGAGCAGGTCTTA |
| | | GTA | | 5 | GGA |
| 932 | E53S13.3G-Cv5 | CTTGTGGATTGCATCTACT | 283 | E53SL1 | AAGGAAGAAGCTGAGCAGG |
| | | GTA | | 6 | TCT |
| 932 | E53S13.3G-Cv5 | CTTGTGGATTGCATCTACT | 284 | E53SL1 | GGAAGCTAAGGAAGAAGCT |
| | | GTA | | 7 | GAG |
| 932 | E53S13.3G-Cv5 | CTTGTGGATTGCATCTACT | 285 | E53SL1 | TTCAACACAATGGCTGGAA |
| | | GTA | | 8 | GCT |
| 932 | E53S13.3G-Cv5 | CTTGTGGATTGCATCTACT | 286 | E53SL1 | GTTAAAGGATTCAACACAA |
| | | GTA | | 9 | TGG |
| 932 | E53S13.3G-Cv5 | CTTGTGGATTGCATCTACT | 287 | E53SL2 | AAATGTTAAAGGATTCAAC |
| | | GTA | | 0 | ACA |
| 932 | E53S13.3G-Cv5 | CTTGTGGATTGCATCTACT | 288 | E53SL2 | GCAACAGTTGAATGAAATG |
| | | GTA | | 1 | TTA |
| 932 | E53S13.3G-Cv5 | CTTGTGGATTGCATCTACT | 289 | E53SL2 | TACAAGAACACCTTCAGAA |
| | | GTA | | 2 | CCG |
| 932 | E53S13.3G-Cv5 | CTTGTGGATTGCATCTACT | 290 | E53SL2 | AAGTACAAGAACACCTTCA |
| | | GTA | | 3 | GAA |
| 932 | E53S13.3G-Cv5 | CTTGTGGATTGCATCTACT | 291 | E53SL2 | AGTTGAAAGAATTCAGAAT |
| | | GTA | | 4 | CAG |
| 932 | E53S13.3G-Cv5 | CTTGTGGATTGCATCTACT | 292 | E53SL2 | TAGTTGAAAGAATTCAGAA |
| | | GTA | | 5 | TCA |
| 933 | E53S13.4A-Cv5 | CTTTAGGATTGCATCTACT | 272 | E53SL4 | TTTTGGATTGCATCTACTG |
| | | GTA | | | TAT |
| 933 | E53S13.4A-Cv5 | CTTTAGGATTGCATCTACT | 273 | E53SL5 | ACCCTCCTTCCATGACTCA |
| | | GTA | | | AGC |
| 933 | E53S13.4A-Cv5 | CTTTAGGATTGCATCTACT | 274 | E53SL6 | CTTCCATGACTCAAGCTTG |
| | | GTA | | | GCT |
| 933 | E53S13.4A-Cv5 | CTTTAGGATTGCATCTACT | 275 | E53SL7 | ACATTTCATTCAACTGTTG |
| | | GTA | | | CCT |
| 933 | E53S13.4A-Cv5 | CTTTAGGATTGCATCTACT | 276 | E53SL8 | TCAACTGTTGCCTCCGGTT |
| | | GTA | | | CTG |
| 933 | E53S13.4A-Cv5 | CTTTAGGATTGCATCTACT | 277 | E53SL1 | CCAAAAGAAAATCACAGAA |
| | | GTA | | 0 | ACC |
| 933 | E53S13.4A-Cv5 | CTTTAGGATTGCATCTACT | 278 | E53SL1 | GCCAAGCTTGAGTCATGGA |
| | | GTA | | 1 | AGG |
| 933 | E53S13.4A-Cv5 | CTTTAGGATTGCATCTACT | 279 | E53SL1 | AGCCAAGCTTGAGTCATGG |
| | | GTA | | 2 | AAG |
| 933 | E53S13.4A-Cv5 | CTTTAGGATTGCATCTACT | 280 | E53SL1 | CAGAGCCAAGCTTGAGTCA |
| | | GTA | | 3 | TGG |
| 933 | E53S13.4A-Cv5 | CTTTAGGATTGCATCTACT | 281 | E53SL1 | AGGCCAGAGCCAAGCTTGA |
| | | GTA | | 4 | GTC |
| 933 | E53S13.4A-Cv5 | CTTTAGGATTGCATCTACT | 282 | E53SL1 | AGAAGCTGAGCAGGTCTTA |
| | | GTA | | 5 | GGA |
| 933 | E53S13.4A-Cv5 | CTTTAGGATTGCATCTACT | 283 | E53SL1 | AAGGAAGAAGCTGAGCAGG |
| | | GTA | | 6 | TCT |
| 933 | E53S13.4A-Cv5 | CTTTAGGATTGCATCTACT | 284 | E53SL1 | GGAAGCTAAGGAAGAAGCT |
| | | GTA | | 7 | GAG |
| 933 | E53S13.4A-Cv5 | CTTTAGGATTGCATCTACT | 285 | E53SL1 | TTCAACACAATGGCTGGAA |
| | | GTA | | 8 | GCT |
| 933 | E53S13.4A-Cv5 | CTTTAGGATTGCATCTACT | 286 | E53SL1 | GTTAAAGGATTCAACACAA |
| | | GTA | | 9 | TGG |
| 933 | E53S13.4A-Cv5 | CTTTAGGATTGCATCTACT | 287 | E53SL2 | AAATGTTAAAGGATTCAAC |
| | | GTA | | 0 | ACA |
| 933 | E53S13.4A-Cv5 | CTTTAGGATTGCATCTACT | 288 | E53SL2 | GCAACAGTTGAATGAAATG |
| | | GTA | | 1 | TTA |
| 933 | E53S13.4A-Cv5 | CTTTAGGATTGCATCTACT | 289 | E53SL2 | TACAAGAACACCTTCAGAA |
| | | GTA | | 2 | CCG |
| 933 | E53S13.4A-Cv5 | CTTTAGGATTGCATCTACT | 290 | E53SL2 | AAGTACAAGAACACCTTCA |
| | | GTA | | 3 | GAA |
| 933 | E53S13.4A-Cv5 | CTTTAGGATTGCATCTACT | 291 | E53SL2 | AGTTGAAAGAATTCAGAAT |
| | | GTA | | 4 | CAG |
| 933 | E53S13.4A-Cv5 | CTTTAGGATTGCATCTACT | 292 | E53SL2 | TAGTTGAAAGAATTCAGAA |
| | | GTA | | 5 | TCA |
| 934 | E53S13.4C-Cv5 | CTTTCGGATTGCATCTACT | 272 | E53SL4 | TTTTGGATTGCATCTACTG |
| | | GTA | | | TAT |
| 934 | E53S13.4C-Cv5 | CTTTCGGATTGCATCTACT | 273 | E53SL5 | ACCCTCCTTCCATGACTCA |
| | | GTA | | | AGC |
| 934 | E53S13.4C-Cv5 | CTTTCGGATTGCATCTACT | 274 | E53SL6 | CTTCCATGACTCAAGCTTG |
| | | GTA | | | GCT |
| 934 | E53S13.4C-Cv5 | CTTTCGGATTGCATCTACT | 275 | E53SL7 | ACATTTCATTCAACTGTTG |
| | | GTA | | | CCT |
| 934 | E53S13.4C-Cv5 | CTTTCGGATTGCATCTACT | 276 | E53SL8 | TCAACTGTTGCCTCCGGTT |
| | | GTA | | | CTG |
| 934 | E53S13.4C-Cv5 | CTTTCGGATTGCATCTACT | 277 | E53SL1 | CCAAAAGAAAATCACAGAA |
| | | GTA | | 0 | ACC |
| 934 | E53S13.4C-Cv5 | CTTTCGGATTGCATCTACT | 278 | E53SL1 | GCCAAGCTTGAGTCATGGA |
| | | GTA | | 1 | AGG |
| 934 | E53S13.4C-Cv5 | CTTTCGGATTGCATCTACT | 279 | E53SL1 | AGCCAAGCTTGAGTCATGG |
| | | GTA | | 2 | AAG |
| 934 | E53S13.4C-Cv5 | CTTTCGGATTGCATCTACT | 280 | E53SL1 | CAGAGCCAAGCTTGAGTCA |
| | | GTA | | 3 | TGG |
| 934 | E53S13.4C-Cv5 | CTTTCGGATTGCATCTACT | 281 | E53SL1 | AGGCCAGAGCCAAGCTTGA |
| | | GTA | | 4 | GTC |
| 934 | E53S13.4C-Cv5 | CTTTCGGATTGCATCTACT | 282 | E53SL1 | AGAAGCTGAGCAGGTCTTA |
| | | GTA | | 5 | GGA |
| 934 | E53S13.4C-Cv5 | CTTTCGGATTGCATCTACT | 283 | E53SL1 | AAGGAAGAAGCTGAGCAGG |
| | | GTA | | 6 | TCT |
| 934 | E53S13.4C-Cv5 | CTTTCGGATTGCATCTACT | 284 | E53SL1 | GGAAGCTAAGGAAGAAGCT |
| | | GTA | | 7 | GAG |
| 934 | E53S13.4C-Cv5 | CTTTCGGATTGCATCTACT | 285 | E53SL1 | TTCAACACAATGGCTGGAA |
| | | GTA | | 8 | GCT |
| 934 | E53S13.4C-Cv5 | CTTTCGGATTGCATCTACT | 286 | E53SL1 | GTTAAAGGATTCAACACAA |
| | | GTA | | 9 | TGG |
| 934 | E53S13.4C-Cv5 | CTTTCGGATTGCATCTACT | 287 | E53SL2 | AAATGTTAAAGGATTCAAC |
| | | GTA | | 0 | ACA |
| 934 | E53S13.4C-Cv5 | CTTTCGGATTGCATCTACT | 288 | E53SL2 | GCAACAGTTGAATGAAATG |
| | | GTA | | 1 | TTA |
| 934 | E53S13.4C-Cv5 | CTTTCGGATTGCATCTACT | 289 | E53SL2 | TACAAGAACACCTTCAGAA |
| | | GTA | | 2 | CCG |
| 934 | E53S13.4C-Cv5 | CTTTCGGATTGCATCTACT | 290 | E53SL2 | AAGTACAAGAACACCTTCA |
| | | GTA | | 3 | GAA |
| 934 | E53S13.4C-Cv5 | CTTTCGGATTGCATCTACT | 291 | E53SL2 | AGTTGAAAGAATTCAGAAT |
| | | GTA | | 4 | CAG |
| 934 | E53S13.4C-Cv5 | CTTTCGGATTGCATCTACT | 292 | E53SL2 | TAGTTGAAAGAATTCAGAA |
| | | GTA | | 5 | TCA |
| 935 | E53S13.4G-Cv5 | CTTTGGGATTGCATCTACT | 272 | E53SL4 | TTTTGGATTGCATCTACTG |
| | | GTA | | | TAT |
| 935 | E53S13.4G-Cv5 | CTTTGGGATTGCATCTACT | 273 | E53SL5 | ACCCTCCTTCCATGACTCA |
| | | GTA | | | AGC |
| 935 | E53S13.4G-Cv5 | CTTTGGGATTGCATCTACT | 274 | E53SL6 | CTTCCATGACTCAAGCTTG |
| | | GTA | | | GCT |
| 935 | E53S13.4G-Cv5 | CTTTGGGATTGCATCTACT | 275 | E53SL7 | ACATTTCATTCAACTGTTG |
| | | GTA | | | CCT |
| 935 | E53S13.4G-Cv5 | CTTTGGGATTGCATCTACT | 276 | E53SL8 | TCAACTGTTGCCTCCGGTT |
| | | GTA | | | CTG |
| 935 | E53S13.4G-Cv5 | CTTTGGGATTGCATCTACT | 277 | E53SL1 | CCAAAAGAAAATCACAGAA |
| | | GTA | | 0 | ACC |
| 935 | E53S13.4G-Cv5 | CTTTGGGATTGCATCTACT | 278 | E53SL1 | GCCAAGCTTGAGTCATGGA |
| | | GTA | | 1 | AGG |
| 935 | E53S13.4G-Cv5 | CTTTGGGATTGCATCTACT | 279 | E53SL1 | AGCCAAGCTTGAGTCATGG |
| | | GTA | | 2 | AAG |
| 935 | E53S13.4G-Cv5 | CTTTGGGATTGCATCTACT | 280 | E53SL1 | CAGAGCCAAGCTTGAGTCA |
| | | GTA | | 3 | TGG |
| 935 | E53S13.4G-Cv5 | CTTTGGGATTGCATCTACT | 281 | E53SL1 | AGGCCAGAGCCAAGCTTGA |
| | | GTA | | 4 | GTC |
| 935 | E53S13.4G-Cv5 | CTTTGGGATTGCATCTACT | 282 | E53SL1 | AGAAGCTGAGCAGGTCTTA |
| | | GTA | | 5 | GGA |
| 935 | E53S13.4G-Cv5 | CTTTGGGATTGCATCTACT | 283 | E53SL1 | AAGGAAGAAGCTGAGCAGG |
| | | GTA | | 6 | TCT |
| 935 | E53S13.4G-Cv5 | CTTTGGGATTGCATCTACT | 284 | E53SL1 | GGAAGCTAAGGAAGAAGCT |
| | | GTA | | 7 | GAG |
| 935 | E53S13.4G-Cv5 | CTTTGGGATTGCATCTACT | 285 | E53SL1 | TTCAACACAATGGCTGGAA |
| | | GTA | | | GCT |
| 935 | E53S13.4G-Cv5 | CTTTGGGATTGCATCTACT | 286 | E53SL1 | GTTAAAGGATTCAACACAA |
| | | GTA | | 9 | TGG |
| 935 | E53S13.4G-Cv5 | CTTTGGGATTGCATCTACT | 287 | E53SL2 | AAATGTTAAAGGATTCAAC |
| | | GTA | | 0 | ACA |
| 935 | E53S13.4G-Cv5 | CTTTGGGATTGCATCTACT | 288 | E53SL2 | GCAACAGTTGAATGAAATG |
| | | GTA | | 1 | TTA |
| 935 | E53S13.4G-Cv5 | CTTTGGGATTGCATCTACT | 289 | E53SL2 | TACAAGAACACCTTCAGAA |
| | | GTA | | 2 | CCG |
| 935 | E53S13.4G-Cv5 | CTTTGGGATTGCATCTACT | 290 | E53SL2 | AAGTACAAGAACACCTTCA |
| | | GTA | | 3 | GAA |
| 935 | E53S13.4G-Cv5 | CTTTGGGATTGCATCTACT | 291 | E53SL2 | AGTTGAAAGAATTCAGAAT |
| | | GTA | | 4 | CAG |
| 935 | E53S13.4G-Cv5 | CTTTGGGATTGCATCTACT | 292 | E53SL2 | TAGTTGAAAGAATTCAGAA |
| | | GTA | | 5 | TCA |
| 936 | E53S13. del2-Cv5 | TTTGGATTGCATCTACTGT | 272 | E53SL4 | TTTTGGATTGCATCTACTG |
| | | A | | | TAT |
| 936 | E53S13. del2-Cv5 | TTTGGATTGCATCTACTGT | 273 | E53SL5 | ACCCTCCTTCCATGACTCA |
| | | A | | | AGC |
| 936 | E53S13. del2-Cv5 | TTTGGATTGCATCTACTGT | 274 | E53SL6 | CTTCCATGACTCAAGCTTG |
| | | A | | | GCT |
| 936 | E53S13.del2-Cv5 | TTTGGATTGCATCTACTGT | 275 | E53SL7 | ACATTTCATTCAACTGTTG |
| | | A | | | CCT |
| 936 | E53S13.del2-Cv5 | TTTGGATTGCATCTACTGT | 276 | E53SL8 | TCAACTGTTGCCTCCGGTT |
| | | A | | | CTG |
| 936 | E53S13.del2-Cv5 | TTTGGATTGCATCTACTGT | 277 | E53SL1 | CCAAAAGAAAATCACAGAA |
| | | A | | 0 | ACC |
| 936 | E53S13.del2-Cv5 | TTTGGATTGCATCTACTGT | 278 | E53SL1 | GCCAAGCTTGAGTCATGGA |
| | | A | | 1 | AGG |
| 936 | E53S13.de12-Cv5 | TTTGGATTGCATCTACTGT | 279 | E53SL1 | AGCCAAGCTTGAGTCATGG |
| | | A | | 2 | AAG |
| 936 | E53S13.del2-Cv5 | TTTGGATTGCATCTACTGT | 280 | E53SL1 | CAGAGCCAAGCTTGAGTCA |
| | | A | | 3 | TGG |
| 936 | E53S13.del2-Cv5 | TTTGGATTGCATCTACTGT | 281 | E53SL1 | AGGCCAGAGCCAAGCTTGA |
| | | A | | 4 | GTC |
| 936 | E53S13.de12-Cv5 | TTTGGATTGCATCTACTGT | 282 | E53SL1 | AGAAGCTGAGCAGGTCT TA |
| | | A | | 5 | GGA |
| 936 | E53S13.del2-Cv5 | TTTGGATTGCATCTACTGT | 283 | E53SL1 | AAGGAAGAAGCTGAGCAGG |
| | | A | | 6 | TCT |
| 936 | E53S13.del2-Cv5 | TTTGGATTGCATCTACTGT | 284 | E53SL1 | GGAAGCTAAGGAAGAAGCT |
| | | A | | 7 | GAG |
| 936 | E53S13.del2-Cv5 | TTTGGATTGCATCTACTGT | 285 | E53SL1 | TTCAACACAATGGCTGGAA |
| | | A | | 8 | GCT |
| 936 | E53S13.de12-Cv5 | TTTGGATTGCATCTACTGT | 286 | E53SL1 | GTTAAAGGATTCAACACAA |
| | | A | | 9 | TGG |
| 936 | E53S13.de12-Cv5 | TTTGGATTGCATCTACTGT | 287 | E53SL2 | AAATGTTAAAGGATTCAAC |
| | | A | | 0 | ACA |
| 936 | E53S13.del2-Cv5 | TTTGGATTGCATCTACTGT | 288 | E53SL2 | GCAACAGTTGAATGAAATG |
| | | A | | 1 | TTA |
| 936 | E53S13 del2-Cv5 | TTTGGATTGCATCTACTGT | 289 | E53SL2 | TACAAGAACACCTTCAGAA |
| | | A | | 2 | CCG |
| 936 | E53S13.del2-Cv5 | TTTGGATTGCATCTACTGT | 290 | E53SL2 | AAGTACAAGAACACCTTCA |
| | | A | | 3 | GAA |
| 936 | E53S13.del2-Cv5 | TTTGGATTGCATCTACTGT | 291 | E53SL2 | AGTTGAAAGAATTCAGAAT |
| | | A | | 4 | CAG |
| 936 | E53S13.de12-Cv5 | TTTGGATTGCATCTACTGT | 292 | E53SL2 | TAGTTGAAAGAATTCAGAA |
| | | A | | 5 | TCA |
| 937 | E53S13.delT-Cv5 | CTTTGGATTGCATCTACTG | 272 | E53SL4 | TTTTGGATTGCATCTACTG |
| | | TA | | | TAT |
| 937 | E53S13.delT-Cv5 | CTTTGGATTGCATCTACTG | 273 | E53SL5 | ACCCTCCTTCCATGACTCA |
| | | TA | | | AGC |
| 937 | E53S13.delT-Cv5 | CTTTGGATTGCATCTACTG | 274 | E53SL6 | CTTCCATGACTCAAGCTTG |
| | | TA | | | GCT |
| 937 | E53S13.delT-Cv5 | CTTTGGATTGCATCTACTG | 275 | E53SL7 | ACATTTCATTCAACTGTTG |
| | | TA | | | CCT |
| 937 | E53S13.delT-Cv5 | CTTTGGATTGCATCTACTG | 276 | E53SL8 | TCAACTGTTGCCTCCGGTT |
| | | TA | | | CTG |
| 937 | E53S13.delT-Cv5 | CTTTGGATTGCATCTACTG | 277 | E53SL1 | CCAAAAGAAAATCACAGAA |
| | | TA | | 0 | ACC |
| 937 | E53S13.delT-Cv5 | CTTTGGATTGCATCTACTG | 278 | E53SL1 | GCCAAGCTTGAGTCATGGA |
| | | TA | | 1 | AGG |
| 937 | E53S13.delT-Cv5 | CTTTGGATTGCATCTACTG | 279 | E53SL1 | AGCCAAGCTTGAGTCATGG |
| | | TA | | 2 | AAG |
| 937 | E53S13.delT-Cv5 | CTTTGGATTGCATCTACTG | 280 | E53SL1 | CAGAGCCAAGCTTGAGTCA |
| | | TA | | 3 | TGG |
| 937 | E53S13.delT-Cv5 | CTTTGGATTGCATCTACTG | 281 | E53SL1 | AGGCCAGAGCCAAGCTTGA |
| | | TA | | 4 | GTC |
| 937 | E53S13.delT-Cv5 | CTTTGGATTGCATCTACTG | 282 | E53SL1 | AGAAGCTGAGCAGGTCTTA |
| | | TA | | 5 | GGA |
| 937 | E53S13.delT-Cv5 | CTTTGGATTGCATCTACTG | 283 | E53SL1 | AAGGAAGAAGCTGAGCAGG |
| | | TA | | 6 | TCT |
| 937 | E53S13.delT-Cv5 | CTTTGGATTGCATCTACTG | 284 | E53SL1 | GGAAGCTAAGGAAGAAGCT |
| | | TA | | 7 | GAG |
| 937 | E53S13.delT-Cv5 | CTTTGGATTGCATCTACTG | 285 | E53SL1 | TTCAACACAATGGCTGGAA |
| | | TA | | 8 | GCT |
| 937 | E53S13.delT-Cv5 | CTTTGGATTGCATCTACTG | 286 | E53SL1 | GTTAAAGGATTCAACACAA |
| | | TA | | 9 | TGG |
| 937 | E53S13.delT-Cv5 | CTTTGGATTGCATCTACTG | 287 | E53SL2 | AAATGTTAAAGGATTCAAC |
| | | TA | | 0 | ACA |
| 937 | E53S13.delT-Cv5 | CTTTGGATTGCATCTACTG | 288 | E53SL2 | GCAACAGTTGAATGAAATG |
| | | TA | | 1 | TTA |
| 937 | E53S13.delT-Cv5 | CTTTGGATTGCATCTACTG | 289 | E53SL2 | TACAAGAACACCTTCAGAA |
| | | TA | | 2 | CCG |
| 937 | E53S13.delT-Cv5 | CTTTGGATTGCATCTACTG | 290 | E53SL2 | AAGTACAAGAACACCTTCA |
| | | TA | | 3 | GAA |
| 937 | E53S13.delT-Cv5 | CTTTGGATTGCATCTACTG | 291 | E53SL2 | AGTTGAAAGAATTCAGAAT |
| | | TA | | 4 | CAG |
| 937 | E53S13.delT-Cv5 | CTTTGGATTGCATCTACTG | 292 | E53SL2 | TAGTTGAAAGAATTCAGAA |
| | | TA | | 5 | TCA |
| 938 | E53S13.delT + A.- | ACTTTGGATTGCATCTACT | 272 | E53SL4 | TTTTGGATTGCATCTACTG |
| | Cv5 | GTA | | | TAT |
| 938 | E53S13.delT + A.- | ACTTTGGATTGCATCTACT | 273 | E53SL5 | ACCCTCCTTCCATGACTCA |
| | Cv5 | GTA | | | AGC |
| 938 | E53S13.delT + A.- | ACTTTGGATTGCATCTACT | 274 | E53SL6 | CTTCCATGACTCAAGCTTG |
| | Cv5 | GTA | | | GCT |
| 938 | E53S13.delT + A.- | ACTTTGGATTGCATCTACT | 275 | E53SL7 | ACATTTCATTCAACTGTTG |
| | Cv5 | GTA | | | CCT |
| 938 | E53S13.delT + A.- | ACTTTGGATTGCATCTACT | 276 | E53SL8 | TCAACTGTTGCCTCCGGTT |
| | Cv5 | GTA | | | CTG |
| 938 | E53S13.delT + A.- | ACTTTGGATTGCATCTACT | 277 | E53SL1 | CCAAAAGAAAATCACAGAA |
| | Cv5 | GTA | | 0 | ACC |
| 938 | E53S13.delT + A.- | ACTTTGGATTGCATCTACT | 278 | E53SL1 | GCCAAGCTTGAGTCATGGA |
| | Cv5 | GTA | | 1 | AGG |
| 938 | E53S13.delT + A.- | ACTTTGGATTGCATCTACT | 279 | E53SL1 | AGCCAAGCTTGAGTCATGG |
| | Cv5 | GTA | | 2 | AAG |
| 938 | E53S13.delT + A.- | ACTTTGGATTGCATCTACT | 280 | E53SL1 | CAGAGCCAAGCTTGAGTCA |
| | Cv5 | GTA | | 3 | TGG |
| 938 | E53S13.delT + A.- | ACTTTGGATTGCATCTACT | 281 | E53SL1 | AGGCCAGAGCCAAGCTTGA |
| | Cv5 | GTA | | 4 | GTC |
| 938 | E53S13.delT + A.- | ACTTTGGATTGCATCTACT | 282 | E53SL1 | AGAAGCTGAGCAGGTCTTA |
| | Cv5 | GTA | | 5 | GGA |
| 938 | E53S13.delT + A.- | ACTTTGGATTGCATCTACT | 283 | E53SL1 | AAGGAAGAAGCTGAGCAGG |
| | Cv5 | GTA | | 6 | TCT |
| 938 | E53S13.delT + A.- | ACTTTGGATTGCATCTACT | 284 | E53SL1 | GGAAGCTAAGGAAGAAGCT |
| | Cv5 | GTA | | 7 | GAG |
| 938 | E53S13.delT + A.- | ACTTTGGATTGCATCTACT | 285 | E53SL1 | TTCAACACAATGGCTGGAA |
| | Cv5 | GTA | | 8 | GCT |
| 938 | E53S13.delT + A.- | ACTTTGGATTGCATCTACT | 286 | E53SL1 | GTTAAAGGATTCAACACAA |
| | Cv5 | GTA | | 9 | TGG |
| 938 | E53S13.delT + A.- | ACTTTGGATTGCATCTACT | 287 | E53SL2 | AAATGTTAAAGGATTCAAC |
| | Cv5 | GTA | | 0 | ACA |
| 938 | E53S13.delT + A.- | ACTTTGGATTGCATCTACT | 288 | E53SL2 | GCAACAGTTGAATGAAATG |
| | Cv5 | GTA | | 1 | TTA |
| 938 | E53S13.delT + A.- | ACTTTGGATTGCATCTACT | 289 | E53SL2 | TACAAGAACACCTTCAGAA |
| | Cv5 | GTA | | 2 | CCG |
| 938 | E53S13.delT + A.- | ACTTTGGATTGCATCTACT | 290 | E53SL2 | AAGTACAAGAACACCTTCA |
| | Cv5 | GTA | | 3 | GAA |
| 938 | E53S13.delT + A.- | ACTTTGGATTGCATCTACT | 291 | E53SL2 | AGTTGAAAGAATTCAGAAT |
| | Cv5 | GTA | | 4 | CAG |
| 938 | E53S13.delT + A.- | ACTTTGGATTGCATCTACT | 292 | E53SL2 | TAGTTGAAAGAATTCAGAA |
| | Cv5 | GTA | | 5 | TCA |
| 950 | E53SL3-21 | TTTTGGATTGCATCTACTG | 275 | E53SL7 | ACATTTCATTCAACTGTTG |
| | | TA | | | CCT |
| 951 | E53SL3-20 | TTTGGATTGCATCTACTGT | 275 | E53SL7 | ACATTTCATTCAACTGTTG |
| | | A | | | CCT |
| 952 | E53SL3-19 | TTGGATTGCATCTACTGTA | 275 | E53SL7 | ACATTTCATTCAACTGTTG |
| | | | | | CCT |
| 953 | E53SL3-18 | TGGATTGCATCTACTGTA | 275 | E53SL7 | ACATTTCATTCAACTGTTG |
| | | | | | CCT |
| 954 | E53SL3-17 | GGATTGCATCTACTGTA | 275 | E53SL7 | ACATTTCATTCAACTGTTG |
| | | | | | CCT |
| 955 | E53SL3-16 | GATTGCATCTACTGTA | 275 | E53SL7 | ACATTTCATTCAACTGTTG |
| | | | | | CCT |

TABLE 6
f) All guide sequences (human-hg38.12)

| Seq ID | Guide RNA Name or Guide ID | | | | | |
|---|---|---|---|---|---|---|
| No. | name | exon_id | enzyme | strand | Guide Sequence | pam |
| 1 | E44Sa1 | 44 | SaCas9 | + | ATTTAGCATGTTCCCAATT | AGGAA |
| | | | | | CTC | T |
| 2 | E44Sa3 | 44 | SaCas9 | − | TCTCAGAAAGACACAAATT | GAGAA |
| | | | | | CCT | T |
| 3 | E45Sa1 | 45 | SaCas9 | + | TCAGGCTTCCCAATTTTTC | TAGAA |
| | | | | | CTG | T |
| 4 | E45Sa2 | 45 | SaCas9 | + | TAGAATACTGGCATCTGTT | GAGGA |
| | | | | | TTT | T |
| 5 | E45Sa3 | 45 | SaCas9 | + | TGGCATCTGTTTTTGAGGA | CTGAA |
| | | | | | TTG | T |
| 6 | E45Sa4 | 45 | SaCas9 | + | TTGCCGCTGCCCAATGCCA | TGGAG |
| | | | | | TCC | T |
| 7 | E45Sa6 | 45 | SaCas9 | − | TCTACAGGAAAAATTGGGA | CTGAA |
| | | | | | AGC | T |
| 8 | E45Sa7 | 45 | SaCas9 | − | GCGGCAAACTGTTGTCAGA | TTGAA |
| | | | | | ACA | T |
| 9 | E45Sa8 | 45 | SaCas9 | − | TTTTGGTATCTTACAGGAA | CAGGA |
| | | | | | CTC | T |
| 10 | E50Sa2 | 50 | SaCas9 | − | AGGAAGTTAGAAGATCTGA | CTGAG |
| | | | | | GCT | T |
| 11 | E51Sa1 | 51 | SaCas9 | + | TAGTAACCACAGGTTGTGT | CAGAG |
| | | | | | CAC | T |
| 12 | E51Sa2 | 51 | SaCas9 | + | GTTGTGTCACCAGAGTAAC | CTGAG |
| | | | | | AGT | T |
| 13 | E51 Sa4 | 51 | SaCas9 | − | GAGGGTGATGGTGGGTGAC | GAGGA |
| | | | | | CTT | T |
| 14 | E51Sa5 | 51 | SaCas9 | − | TATAAAATCACAGAGGGTG | GTGGG |
| | | | | | ATG | T |
| 15 | E51Sa6 | 51 | SaCas9 | − | TTGATCAAGTTATAAAATC | GAGGG |
| | | | | | ACA | T |
| 16 | E53Sa1 | 53 | SaCas9 | + | CCTTGGTTTCTGTGATTTT | TTGGA |
| | | | | | CTT | T |
| 17 | E53Sa2 | 53 | SaCas9 | + | TCCTTAGCTTCCAGCCATT | TTGAA |
| | | | | | GTG | T |
| 18 | E53Sa3 | 53 | SaCas9 | + | CTTGTACTTCATCCCACTG | CTGAA |
| | | | | | ATT | T |
| 19 | E53Sa4 | 53 | SaCas9 | + | ACTGATTCTGAATTCTTTC | TAGAA |
| | | | | | AAC | T |
| 20 | E53Sa5 | 53 | SaCas9 | − | AGCCAAGCTTGAGTCATGG | GAGGG |
| | | | | | AAG | T |
| 21 | E53Sa6 | 53 | SaCas9 | − | TTAGGACAGGCCAGAGCCA | TTGAG |
| | | | | | AGC | T |
| 22 | E53Sa7 | 53 | SaCas9 | − | GCAACAGTTGAATGAAATG | AAGGA |
| | | | | | TTA | T |
| 23 | E53Sa8 | 53 | SaCas9 | − | CCTTCAGAACCGGAGGCAA | TTGAA |
| | | | | | CAG | T |
| 24 | E53Sa9 | 53 | SaCas9 | − | AGTTGAAAGAATTCAGAAT | TGGGA |
| | | | | | CAG | T |
| 25 | E53Sa10 | 53 | SaCas9 | − | TTTTATTCTAGTTGAAAGA | CAGAA |
| | | | | | ATT | T |
| 26 | E53Sa11 | 53 | SaCas9 | − | TTTTTCCTTTTATTCTAGT | AAGAA |
| | | | | | TGA | T |
| 27 | E51SaCas9KKH1 | 51 | SACAS9 | + | TCATTTTTTCTCATACCTT | CTTGA |
| | | | KKH | | CTG | T |
| 28 | E51SaCas9KKH2 | 51 | SACAS9 | + | TTTTTTCTCATACCTTCTG | GATGA |
| | | | KKH | | CTT | T |
| 29 | E51SaCas9KKH3 | 51 | SACAS9 | + | CCTTCTGCTTGATGATCAT | GTTGA |
| | | | KKH | | CTC | T |
| 30 | E51SaCas9KKH4 | 51 | SACAS9 | + | ATGATCATCTCGTTGATAT | CAAGG |
| | | | KKH | | CCT | T |
| 31 | E51SaCas9KKH5 | 51 | SACAS9 | + | AAGGTCACCCACCATCACC | TGTGA |
| | | | KKH | | CTC | T |
| 32 | E51SaCas9KKH6 | 51 | SACAS9 | + | ATCACCCTCTGTGATTTTA | CTTGA |
| | | | KKH | | TAA | T |
| 33 | E51SaCas9KKH7 | 51 | SACAS9 | + | ATAACTTGATCAAGCAGAG | GCCAG |
| | | | KKH | | AAA | T |
| 34 | E51SaCas9KKH8 | 51 | SACAS9 | + | CTTGATCAAGCAGAGAAAG | GTCGG |
| | | | KKH | | CCA | T |
| 35 | E51SaCas9KKH9 | 51 | SACAS9 | + | ATCAAGCAGAGAAAGCCAG | GTAAG |
| | | | KKH | | TCG | T |
| 36 | E51SaCas9KKH10 | 51 | SACAS9 | + | CAGTCGGTAAGTTCTGTCC | CCCGG |
| | | | KKH | | AAG | T |
| 37 | E51SaCas9KKH11 | 51 | SACAS9 | + | GTAAGTTCTGTCCAAGCCC | TGAAA |
| | | | KKH | | GGT | T |
| 38 | E51SaCas9KKH12 | 51 | SACAS9 | + | AGCCCGGTTGAAATCTGCC | GCAGG |
| | | | KKH | | AGA | T |
| 39 | E51SaCas9KKH13 | 51 | SACAS9 | + | AGCAGGTACCTCCAACATC | GAAGA |
| | | | KKH | | AAG | T |
| 40 | E51SaCas9KKH14 | 51 | SACAS9 | + | CAACATCAAGGAAGATGGC | TCTAG |
| | | | KKH | | ATT | T |
| 41 | E51SaCas9KKH15 | 51 | SACAS9 | + | AGGAAGATGGCATTTCTAG | GGAGA |
| | | | KKH | | TTT | T |
| 42 | E51SaCas9KKH16 | 51 | SACAS9 | + | ATGGCATTTCTAGTTTGGA | GGCAG |
| | | | KKH | | GAT | T |
| 43 | E51SaCas9KKH17 | 51 | SACAS9 | + | CTAGTTTGGAGATGGCAGT | CTTAG |
| | | | KKH | | TTC | T |
| 44 | E51SaCas9KKH18 | 51 | SACAS9 | + | GATGGCAGTTTCCTTAGTA | ACAGG |
| | | | KKH | | ACC | T |
| 45 | E51SaCas9KKH19 | 51 | SACAS9 | + | TAGTAACCACAGGTTGTGT | CAGAG |
| | | | KKH | | CAC | T |
| 46 | E51SaCas9KKH20 | 51 | SACAS9 | + | CCACAGGTTGTGTCACCAG | AACAG |
| | | | KKH | | AGT | T |
| 47 | E51SaCas9KKH21 | 51 | SACAS9 | + | GTTGTGTCACCAGAGTAAC | CTGAG |
| | | | KKH | | AGT | T |
| 48 | E51SaCas9KKH22 | 51 | SACAS9 | + | GAGTAACAGTCTGAGTAGG | TAAAA |
| | | | KKH | | AGC | T |
| 49 | E51SaCas9KKH23 | 51 | SACAS9 | + | TCTGAGTAGGAGCTAAAAT | TTGGG |
| | | | KKH | | ATT | T |
| 50 | E51SaCas9KKH24 | 51 | SACAS9 | − | AGAAGGTATGAGAAAAAAT | AAAAG |
| | | | KKH | | GAT | T |
| 51 | E51SaCas9KKH25 | 51 | SACAS9 | − | TCAAGCAGAAGGTATGAGA | AATGA |
| | | | KKH | | AAA | T |
| 52 | E51SaCas9KKH26 | 51 | SACAS9 | − | TCATCAAGCAGAAGGTATG | AAAAA |
| | | | KKH | | AGA | T |
| 53 | E51SaCas9KKH27 | 51 | SACAS9 | − | TCAACGAGATGATCATCAA | GAAGG |
| | | | KKH | | GCA | T |
| 54 | E51SaCas9KKH28 | 51 | SACAS9 | − | GTGACCTTGAGGATATCAA | GATGA |
| | | | KKH | | CGA | T |
| 55 | E51SaCas9KKH29 | 51 | SACAS9 | − | TGGGTGACCTTGAGGATAT | CGAGA |
| | | | KKH | | CAA | T |
| 56 | E51SaCas9KKH30 | 51 | SACAS9 | − | GAGGGTGATGGTGGGTGAC | GAGGA |
| | | | KKH | | CTT | T |
| 57 | E51SaCas9KKH31 | 51 | SACAS9 | − | TATAAAATCACAGAGGGTG | GTGGG |
| | | | KKH | | ATG | T |
| 58 | E51SaCas9KKH32 | 51 | SACAS9 | − | AAGTTATAAAATCACAGAG | GATGG |
| | | | KKH | | GGT | T |
| 59 | E51SaCas9KKH33 | 51 | SACAS9 | − | ATCAAGTTATAAAATCACA | GGTGA |
| | | | KKH | | GAG | T |
| 60 | E51SaCas9KKH34 | 51 | SACAS9 | − | TTGATCAAGTTATAAAATC | GAGGG |
| | | | KKH | | ACA | T |
| 61 | E51SaCas9KKH35 | 51 | SACAS9 | − | CTTTCTCTGCTTGATCAAG | TAAAA |
| | | | KKH | | TTA | T |
| 62 | E51SaCas9KKH36 | 51 | SACAS9 | − | CCGACTGGCTTTCTCTGCT | TCAAG |
| | | | KKH | | TGA | T |
| 63 | E51SaCas9KKH37 | 51 | SACAS9 | − | ACTTACCGACTGGCTTTCT | CTTGA |
| | | | KKH | | CTG | T |
| 64 | E51SaCas9KKH38 | 51 | SACAS9 | − | GATGTTGGAGGTACCTGCT | GCAGA |
| | | | KKH | | CTG | T |
| 65 | E51SaCas9KKH39 | 51 | SACAS9 | − | AAATGCCATCTTCCTTGAT | GGAGG |
| | | | KKH | | GTT | T |
| 66 | E51SaCas9KKH40 | 51 | SACAS9 | − | CCAAACTAGAAATGCCATC | CTTGA |
| | | | KKH | | TTC | T |
| 67 | E51SaCas9KKH41 | 51 | SACAS9 | − | AGGAAACTGCCATCTCCAA | AGAAA |
| | | | KKH | | ACT | T |
| 68 | E51SaCas9KKH42 | 51 | SACAS9 | − | CTGTTACTCTGGTGACACA | TGTGG |
| | | | KKH | | ACC | T |
| 69 | E51SaCas9KKH43 | 51 | SACAS9 | − | TAGCTCCTACTCAGACTGT | TCTGG |
| | | | KKH | | TAC | T |
| 70 | E53SaCas9KKH1 | 53 | SACAS9 | + | AAAAGGTATCTTTGATACT | CTTGG |
| | | | KKH | | AAC | T |
| 71 | E53SaCas9KKH2 | 53 | SACAS9 | + | CTTTGATACTAACCTTGGT | TGTGA |
| | | | KKH | | TTC | T |
| 72 | E53SaCas9KKH3 | 53 | SACAS9 | + | CCTTGGTTTCTGTGATTTT | TTGGA |
| | | | KKH | | CTT | T |
| 73 | E53SaCas9KKH4 | 53 | SACAS9 | + | TCCTTAGCTTCCAGCCATT | TTGAA |
| | | | KKH | | GTG | T |
| 74 | E53SaCas9KKH5 | 53 | SACAS9 | + | AACATTTCATTCAACTGTT | TCCGG |
| | | | KKH | | GCC | T |
| 75 | E53SaCas9KKH6 | 53 | SACAS9 | + | TTCAACTGTTGCCTCCGGT | GAAGG |
| | | | KKH | | TCT | T |
| 76 | E53SaCas9KKH7 | 53 | SACAS9 | + | AGGTGTTCTTGTACTTCAT | ACTGA |
| | | | KKH | | CCC | T |
| 77 | E53SaCas9KKH8 | 53 | SACAS9 | + | CTTGTACTTCATCCCACTG | CTGAA |
| | | | KKH | | ATT | T |
| 78 | E53SaCas9KKH9 | 53 | SACAS9 | + | ACTGATTCTGAATTCTTTC | TAGAA |
| | | | KKH | | AAC | T |
| 79 | E53SaCas9KKH10 | 53 | SACAS9 | + | TTCTTTCAACTAGAATAAA | AAAAA |
| | | | KKH | | AGG | T |
| 80 | E53SaCas9KKH11 | 53 | SACAS9 | + | TTCAACTAGAATAAAAGGA | ATAAA |
| | | | KKH | | AAA | T |
| 81 | E53SaCas9KKH12 | 53 | SACAS9 | + | AGAATAAAAGGAAAAATAA | TATAG |
| | | | KKH | | ATA | T |
| 82 | E53SaCas9KKH13 | 53 | SACAS9 | − | GTTAGTATCAAAGATACCT | TAAAA |
| | | | KKH | | TTT | T |
| 83 | E53SaCas9KKH14 | 53 | SACAS9 | − | CACAGAAACCAAGGTTAGT | AAAGA |
| | | | KKH | | ATC | T |
| 84 | E53SaCas9KKH15 | 53 | SACAS9 | − | AAAGAAAATCACAGAAACC | GTTAG |
| | | | KKH | | AAG | T |
| 85 | E53SaCas9KKH16 | 53 | SACAS9 | − | TCCAAAAGAAAATCACAGA | CAAGG |
| | | | KKH | | AAC | T |
| 86 | E53SaCas9KKH17 | 53 | SACAS9 | − | ATACAGTAGATGCAATCCA | GAAAA |
| | | | KKH | | AAA | T |
| 87 | E53SaCas9KKH18 | 53 | SACAS9 | − | AGGAGGGTCCCTATACAGT | TGCAA |
| | | | KKH | | AGA | T |
| 88 | E53SaCas9KKH19 | 53 | SACAS9 | − | ATGGAAGGAGGGTCCCTAT | GTAGA |
| | | | KKH | | ACA | T |
| 89 | E53SaCas9KKH20 | 53 | SACAS9 | − | AGTCATGGAAGGAGGGTCC | TACAG |
| | | | KKH | | CTA | T |
| 90 | E53SaCas9KKH21 | 53 | SACAS9 | − | AGCCAAGCTTGAGTCATGG | GAGGG |
| | | | KKH | | AAG | T |
| 91 | E53SaCas9KKH22 | 53 | SACAS9 | − | TTAGGACAGGCCAGAGCCA | TTGAG |
| | | | KKH | | AGC | T |
| 92 | E53SaCas9KKH23 | 53 | SACAS9 | − | TGGAAGCTAAGGAAGAAGC | GCAGG |
| | | | KKH | | TGA | T |
| 93 | E53SaCas9KKH24 | 53 | SACAS9 | − | AATGAAATGTTAAAGGATT | CACAA |
| | | | KKH | | CAA | T |
| 94 | E53SaCas9KKH25 | 53 | SACAS9 | − | GCAACAGTTGAATGAAATG | AAGGA |
| | | | KKH | | TTA | T |
| 95 | E53SaCas9KKH26 | 53 | SACAS9 | − | AGAACCGGAGGCAACAGTT | TGAAA |
| | | | KKH | | GAA | T |
| 96 | E53SaCas9KKH27 | 53 | SACAS9 | − | CCTTCAGAACCGGAGGCAA | TTGAA |
| | | | KKH | | CAG | T |
| 97 | E53SaCas9KKH28 | 53 | SACAS9 | − | GAACACCTTCAGAACCGGA | AACAG |
| | | | KKH | | GGC | T |
| 98 | E53SaCas9KKH29 | 53 | SACAS9 | − | AAAGAATTCAGAATCAGTG | TGAAG |
| | | | KKH | | GGA | T |
| 99 | E53SaCas9KKH30 | 53 | SACAS9 | − | AGTTGAAAGAATTCAGAAT | TGGGA |
| | | | KKH | | CAG | T |
| 100 | E53SaCas9KKH31 | 53 | SACAS9 | − | ATTCTAGTTGAAAGAATTC | ATCAG |
| | | | KKH | | AGA | T |
| 101 | E53SaCas9KKH32 | 53 | SACAS9 | − | TTTTATTCTAGTTGAAAGA | CAGAA |
| | | | KKH | | ATT | T |
| 102 | E53SaCas9KKH33 | 53 | SACAS9 | − | TTTTTCCTTTTATTCTAGT | AAGAA |
| | | | KKH | | TGA | T |
| 103 | E53SaCas9KKH34 | 53 | SACAS9 | − | ATATATTTATTTTTCCTTT | TCTAG |
| | | | KKH | | TAT | T |
| 200 | E44Slu1 | 44 | SluCas9 | + | TATTTAGCATGTTCCCAAT | CAGG |
| | | | | | TCT | |
| 201 | E44Slu2 | 44 | SluCas9 | + | AACAGATCTGTCAAATCGC | CAGG |
| | | | | | CTG | |
| 202 | E44Slu4 | 44 | SluCas9 | − | TGCTAAATACAAATGGTAT | AAGG |
| | | | | | CTT | |
| 203 | E44Slu5 | 44 | SluCas9 | − | ATTGGGAACATGCTAAATA | ATGG |
| | | | | | CAA | |
| 204 | E44Slu6 | 44 | SluCas9 | − | AAAGACACAAATTCCTGAG | TGGG |
| | | | | | AAT | |
| 205 | E44Slu7 | 44 | SluCas9 | − | GAAAGACACAAATTCCTGA | TTGG |
| | | | | | GAA | |
| 206 | E44Slu8 | 44 | SluCas9 | − | ATGATATAAAGATATTTAA | GTGG |
| | | | | | TCA | |
| 207 | E44Slu9 | 44 | SluCas9 | − | TTGACAGATCTGTTGAGAA | GCGG |
| | | | | | ATG | |
| 208 | E44Slu10 | 44 | SluCas9 | − | GATTTGACAGATCTGTTGA | ATGG |
| | | | | | GAA | |
| 209 | E45Slu1 | 45 | SluCas9 | + | AGACCTCCTGCCACCGCAG | CAGG |
| | | | | | ATT | |
| 210 | E45Slu2 | 45 | SluCas9 | + | TCCCAATTTTTCCTGTAGA | CTGG |
| | | | | | ATA | |
| 211 | E45Slu3 | 45 | SluCas9 | + | TAGAATACTGGCATCTGTT | GAGG |
| | | | | | TTT | |
| 212 | E45Slu4 | 45 | SluCas9 | + | TTTGCCGCTGCCCAATGCC | CTGG |
| | | | | | ATC | |
| 213 | E45Slu7 | 45 | SluCas9 | − | CTGTCAGACAGAAAAAAGA | AGGG |
| | | | | | GGT | |
| 214 | E45Slu8 | 45 | SluCas9 | − | GCTGTCAGACAGAAAAAAG | TAGG |
| | | | | | AGG | |
| 215 | E45Slu9 | 45 | SluCas9 | − | AACAGCTGTCAGACAGAAA | GAGG |
| | | | | | AAA | |
| 216 | E45Slu10 | 45 | SluCas9 | − | AAGCCTGAATCTGCGGTGG | GAGG |
| | | | | | CAG | |
| 217 | E45Slu11 | 45 | SluCas9 | − | GGGAAGCCTGAATCTGCGG | CAGG |
| | | | | | TGG | |
| 218 | E45Slu12 | 45 | SluCas9 | − | AATTGGGAAGCCTGAATCT | GTGG |
| | | | | | GCG | |
| 219 | E45Slu13 | 45 | SluCas9 | − | AAAAATTGGGAAGCCTGAA | GCGG |
| | | | | | TCT | |
| 220 | E45Slu14 | 45 | SluCas9 | − | GCCAGTATTCTACAGGAAA | TGGG |
| | | | | | AAT | |
| 221 | E45Slu15 | 45 | SluCas9 | − | TGCCAGTATTCTACAGGAA | TTGG |
| | | | | | AAA | |
| 222 | E45Slu16 | 45 | SluCas9 | − | AAAAACAGATGCCAGTATT | CAGG |
| | | | | | CTA | |
| 223 | E45Slu17 | 45 | SluCas9 | − | TGTCAGAACATTGAATGCA | GGGG |
| | | | | | ACT | |
| 224 | E45Slu18 | 45 | SluCas9 | − | TTGTCAGAACATTGAATGC | TGGG |
| | | | | | AAC | |
| 225 | E45Slu19 | 45 | SluCas9 | − | GTTGTCAGAACATTGAATG | CTGG |
| | | | | | CAA | |
| 226 | E45Slu20 | 45 | SluCas9 | − | AACTCCAGGATGGCATTGG | GCGG |
| | | | | | GCA | |
| 227 | E45Slu21 | 45 | SluCas9 | − | TACAGGAACTCCAGGATGG | TGGG |
| | | | | | CAT | |
| 228 | E45Slu22 | 45 | SluCas9 | − | TTACAGGAACTCCAGGATG | TTGG |
| | | | | | GCA | |
| 229 | E45Slu23 | 45 | SluCas9 | − | GGTATCTTACAGGAACTCC | ATGG |
| | | | | | AGG | |
| 230 | E45Slu24 | 45 | SluCas9 | − | TTTTGGTATCTTACAGGAA | CAGG |
| | | | | | CTC | |
| 231 | E50Slu2 | 50 | SluCas9 | + | AGTATACTTACAGGCTCCA | GTGG |
| | | | | | ATA | |
| 232 | E50Slu3 | 50 | SluCas9 | + | CAGGCTCCAATAGTGGTCA | CAGG |
| | | | | | GTC | |
| 233 | E50Slu4 | 50 | SluCas9 | + | CAATAGTGGTCAGTCCAGG | TAGG |
| | | | | | AGC | |
| 234 | E50Slu5 | 50 | SluCas9 | + | GTGGTCAGTCCAGGAGCTA | CAGG |
| | | | | | GGT | |
| 235 | E50Slu6 | 50 | SluCas9 | + | TTGCCCTCAGCTCTTGAAG | ACGG |
| | | | | | TAA | |
| 236 | E50Slu9 | 50 | SluCas9 | − | CTAGCTCCTGGACTGACCA | TTGG |
| | | | | | CTA | |
| 237 | E50Slu10 | 50 | SluCas9 | − | GCAAAGCAGCCTGACCTAG | CTGG |
| | | | | | CTC | |
| 238 | E50Slu11 | 50 | SluCas9 | − | AAACCGTTTACTTCAAGAG | AGGG |
| | | | | | CTG | |
| 239 | E50Slu12 | 50 | SluCas9 | − | TAAACCGTTTACTTCAAGA | GAGG |
| | | | | | GCT | |
| 240 | E50Slu13 | 50 | SluCas9 | − | AGATCTGAGCTCTGAGTGG | GCGG |
| | | | | | AAG | |
| 241 | E50Slu14 | 50 | SluCas9 | − | AGAAGATCTGAGCTCTGAG | AAGG |
| | | | | | TGG | |
| 242 | E50Slu15 | 50 | SluCas9 | − | AGTTAGAAGATCTGAGCTC | GTGG |
| | | | | | TGA | |
| 243 | E51Slu1 | 51 | SluCas9 | + | TGATCATCTCGTTGATATC | AAGG |
| | | | | | CTC | |
| 244 | E51Slu2 | 51 | SluCas9 | + | TTGATCAAGCAGAGAAAGC | TCGG |
| | | | | | CAG | |
| 245 | E51Slu3 | 51 | SluCas9 | + | AGTCGGTAAGTTCTGTCCA | CCGG |
| | | | | | AGC | |
| 246 | E51Slu4 | 51 | SluCas9 | + | GCCCGGTTGAAATCTGCCA | CAGG |
| | | | | | GAG | |
| 247 | E51Slu5 | 51 | SluCas9 | + | CAGAGCAGGTACCTCCAAC | AAGG |
| | | | | | ATC | |
| 248 | E51Slu6 | 51 | SluCas9 | + | GGTACCTCCAACATCAAGG | ATGG |
| | | | | | AAG | |
| 249 | E51Slu7 | 51 | SluCas9 | + | CAAGGAAGATGGCATTTCT | TTGG |
| | | | | | AGT | |
| 250 | E51Slu8 | 51 | SluCas9 | + | AGATGGCATTTCTAGTTTG | ATGG |
| | | | | | GAG | |
| 251 | E51Slu9 | 51 | SluCas9 | + | ATGGCAGTTTCCTTAGTAA | CAGG |
| | | | | | CCA | |
| 252 | E51Slu10 | 51 | SluCas9 | + | GTCACCAGAGTAACAGTCT | TAGG |
| | | | | | GAG | |
| 253 | E51Slu15 | 51 | SluCas9 | − | CAACGAGATGATCATCAAG | AAGG |
| | | | | | CAG | |
| 254 | E51Slu16 | 51 | SluCas9 | − | GAGGGTGATGGTGGGTGAC | GAGG |
| | | | | | CTT | |
| 255 | E51Slu17 | 51 | SluCas9 | − | ATAAAATCACAGAGGGTGA | TGGG |
| | | | | | TGG | |
| 256 | E51Slu18 | 51 | SluCas9 | − | TATAAAATCACAGAGGGTG | GTGG |
| | | | | | ATG | |
| 257 | E51Slu19 | 51 | SluCas9 | − | AGTTATAAAATCACAGAGG | ATGG |
| | | | | | GTG | |
| 258 | E51Slu20 | 51 | SluCas9 | − | TGATCAAGTTATAAAATCA | AGGG |
| | | | | | CAG | |
| 259 | E51Slu21 | 51 | SluCas9 | − | TTGATCAAGTTATAAAATC | GAGG |
| | | | | | ACA | |
| 260 | E51Slu22 | 51 | SluCas9 | − | GGGCTTGGACAGAACTTAC | CTGG |
| | | | | | CGA | |
| 261 | E51Slu23 | 51 | SluCas9 | − | CTCTGGCAGATTTCAACCG | TTGG |
| | | | | | GGC | |
| 262 | E51Slu24 | 51 | SluCas9 | − | ACCTGCTCTGGCAGATTTC | CGGG |
| | | | | | AAC | |
| 263 | E51Slu25 | 51 | SluCas9 | − | TACCTGCTCTGGCAGATTT | CCGG |
| | | | | | CAA | |
| 264 | E51Slu26 | 51 | SluCas9 | − | CTTGATGTTGGAGGTACCT | CTGG |
| | | | | | GCT | |
| 265 | E51Slu27 | 51 | SluCas9 | − | AATGCCATCTTCCTTGATG | GAGG |
| | | | | | TTG | |
| 266 | E51Slu28 | 51 | SluCas9 | − | AGAAATGCCATCTTCCTTG | TTGG |
| | | | | | ATG | |
| 267 | E51Slu29 | 51 | SluCas9 | − | GGTGACACAACCTGTGGTT | AAGG |
| | | | | | ACT | |
| 268 | E51Slu30 | 51 | SluCas9 | − | TGTTACTCTGGTGACACAA | GTGG |
| | | | | | CCT | |
| 269 | E51Slu31 | 51 | SluCas9 | − | AGCTCCTACTCAGACTGTT | CTGG |
| | | | | | ACT | |
| 270 | E53Slu2 | 53 | SluCas9 | + | CCTTGGTTTCTGTGATTTT | TTGG |
| | | | | | CTT | |
| 271 | E53Slu3 | 53 | SluCas9 | + | CTTTTGGATTGCATCTACT | TAGG |
| | | | | | GTA | |
| 272 | E53Slu4 | 53 | SluCas9 | + | TTTTGGATTGCATCTACTG | AGGG |
| | | | | | TAT | |
| 273 | E53Slu5 | 53 | SluCas9 | + | ACCCTCCTTCCATGACTCA | TTGG |
| | | | | | AGC | |
| 274 | E53Slu6 | 53 | SluCas9 | + | CTTCCATGACTCAAGCTTG | CTGG |
| | | | | | GCT | |
| 275 | E53Slu7 | 53 | SluCas9 | + | ACATTTCATTCAACTGTTG | CCGG |
| | | | | | CCT | |
| 276 | E53Slu8 | 53 | SluCas9 | + | TCAACTGTTGCCTCCGGTT | AAGG |
| | | | | | CTG | |
| 277 | E53Slu10 | 53 | SluCas9 | − | CCAAAAGAAAATCACAGAA | AAGG |
| | | | | | ACC | |
| 278 | E53Slu11 | 53 | SluCas9 | − | GCCAAGCTTGAGTCATGGA | AGGG |
| | | | | | AGG | |
| 279 | E53Slu12 | 53 | SluCas9 | − | AGCCAAGCTTGAGTCATGG | GAGG |
| | | | | | AAG | |
| 280 | E53Slu13 | 53 | SluCas9 | − | CAGAGCCAAGCTTGAGTCA | AAGG |
| | | | | | TGG | |
| 281 | E53Slu14 | 53 | SluCas9 | − | AGGCCAGAGCCAAGCTTGA | ATGG |
| | | | | | GTC | |
| 282 | E53Slu15 | 53 | SluCas9 | − | AGAAGCTGAGCAGGTCTTA | CAGG |
| | | | | | GGA | |
| 283 | E53Slu16 | 53 | SluCas9 | − | AAGGAAGAAGCTGAGCAGG | TAGG |
| | | | | | TCT | |
| 284 | E53Slu17 | 53 | SluCas9 | − | GGAAGCTAAGGAAGAAGCT | CAGG |
| | | | | | GAG | |
| 285 | E53Slu18 | 53 | SluCas9 | − | TTCAACACAATGGCTGGAA | AAGG |
| | | | | | GCT | |
| 286 | E53Slu19 | 53 | SluCas9 | − | GTTAAAGGATTCAACACAA | CTGG |
| | | | | | TGG | |
| 287 | E53Slu20 | 53 | SluCas9 | − | AAATGTTAAAGGATTCAAC | ATGG |
| | | | | | ACA | |
| 288 | E53Slu21 | 53 | SluCas9 | − | GCAACAGTTGAATGAAATG | AAGG |
| | | | | | TTA | |
| 289 | E53Slu22 | 53 | SluCas9 | − | TACAAGAACACCTTCAGAA | GAGG |
| | | | | | CCG | |
| 290 | E53Slu23 | 53 | SluCas9 | − | AAGTACAAGAACACCTTCA | CCGG |
| | | | | | GAA | |
| 291 | E53Slu24 | 53 | SluCas9 | − | AGTTGAAAGAATTCAGAAT | TGGG |
| | | | | | CAG | |
| 292 | E53Slu25 | 53 | SluCas9 | − | TAGTTGAAAGAATTCAGAA | GTGG |
| | | | | | TCA | |
| 104 | E44SaCas9KKH2 | 44 | SaCas9 | + | TACCATTTGTATTTAGCAT | CCCAA |
| | | | KKH | | GTT | T |
| 105 | E44SaCas9KKH3 | 44 | SaCas9 | + | ATTTAGCATGTTCCCAATT | AGGAA |
| | | | KKH | | CTC | T |
| 106 | E44SaCas9KKH4 | 44 | SaCas9 | + | AACTGTTCAGCTTCTGTTA | ACTGA |
| | | | KKH | | GCC | T |
| 107 | E44SaCas9KKH5 | 44 | SaCas9 | + | TTCAGCTTCTGTTAGCCAC | TTAAA |
| | | | KKH | | TGA | T |
| 108 | E44SaCas9KKH6 | 44 | SaCas9 | + | CACTGATTAAATATCTTTA | CATAA |
| | | | KKH | | TAT | T |
| 109 | E44SaCas9KKH7 | 44 | SaCas9 | + | ATGAAAACGCCGCCATTTC | ACAGA |
| | | | KKH | | TCA | T |
| 110 | E44SaCas9KKH8 | 44 | SaCas9 | + | CCGCCATTTCTCAACAGAT | TCAAA |
| | | | KKH | | CTG | T |
| 111 | E44SaCas9KKH9 | 44 | SaCas9 | + | CAACAGATCTGTCAAATCG | GCAGG |
| | | | KKH | | CCT | T |
| 112 | E44SaCas9KKH14 | 44 | SaCas9 | − | TAAATACAAATGGTATCTT | GTAAG |
| | | | KKH | | AAG | T |
| 113 | E44SaCas9KKH15 | 44 | SaCas9 | − | ATGCTAAATACAAATGGTA | TAAGG |
| | | | KKH | | TCT | T |
| 114 | E44SaCas9KKH16 | 44 | SaCas9 | − | AATTGGGAACATGCTAAAT | AATGG |
| | | | KKH | | ACA | T |
| 115 | E44SaCas9KKH17 | 44 | SaCas9 | − | GAGAATTGGGAACATGCTA | ACAAA |
| | | | KKH | | AAT | T |
| 116 | E44SaCas9KKH18 | 44 | SaCas9 | − | ATTCCTGAGAATTGGGAAC | CTAAA |
| | | | KKH | | ATG | T |
| 117 | E44SaCas9KKH19 | 44 | SaCas9 | − | TCTCAGAAAGACACAAATT | GAGAA |
| | | | KKH | | CCT | T |
| 118 | E44SaCas9KKH20 | 44 | SaCas9 | − | CTGAACAGTTTCTCAGAAA | ACAAA |
| | | | KKH | | GAC | T |
| 119 | E44SaCas9KKH21 | 44 | SaCas9 | − | AATCAGTGGCTAACAGAAG | AACAG |
| | | | KKH | | CTG | T |
| 120 | E44SaCas9KKH22 | 44 | SaCas9 | − | CATTATGATATAAAGATAT | ATCAG |
| | | | KKH | | TTA | T |
| 121 | E44SaCas9KKH23 | 44 | SaCas9 | − | TTTTCATTATGATATAAAG | TTTAA |
| | | | KKH | | ATA | T |
| 122 | E44SaCas9KKH24 | 44 | SaCas9 | − | GGCGGCGTTTTCATTATGA | AAAGA |
| | | | KKH | | TAT | T |
| 123 | E44SaCas9KKH25 | 44 | SaCas9 | − | TGAGAAATGGCGGCGTTTT | TATGA |
| | | | KKH | | CAT | T |
| 124 | E44SaCas9KKH26 | 44 | SaCas9 | − | AGGCGATTTGACAGATCTG | AGAAA |
| | | | KKH | | TTG | T |
| 125 | E44SaCas9KKH27 | 44 | SaCas9 | − | GCTTTTACCTGCAGGCGAT | ACAGA |
| | | | KKH | | TTG | T |
| 126 | E45SaCas9KKH1 | 45 | SaCas9 | + | TGTTTGCAGACCTCCTGCC | GCAGA |
| | | | KKH | | ACC | T |
| 127 | E45SaCas9KKH2 | 45 | SaCas9 | + | CTGCCACCGCAGATTCAGG | CCCAA |
| | | | KKH | | CTT | T |
| 128 | E45SaCas9KKH3 | 45 | SaCas9 | + | TCAGGCTTCCCAATTTTTC | TAGAA |
| | | | KKH | | CTG | T |
| 129 | E45SaCas9KKH4 | 45 | SaCas9 | + | TAGAATACTGGCATCTGTT | GAGGA |
| | | | KKH | | TTT | T |
| 130 | E45SaCas9KKH5 | 45 | SaCas9 | 1 | TGGCATCTGTTTTTGAGGA | CTGAA |
| | | | KKH | | TTG | T |
| 131 | E45SaCas9KKH6 | 45 | SaCas9 | + | AGGATTGCTGAATTATTTC | CCCAG |
| | | | KKH | | TTC | T |
| 132 | E45SaCas9KKH7 | 45 | SaCas9 | + | AATTATTTCTTCCCCAGTT | TTCAA |
| | | | KKH | | GCA | T |
| 133 | E45SaCas9KKH8 | 45 | SaCas9 | + | CAGTTGCATTCAATGTTCT | AACAG |
| | | | KKH | | GAC | T |
| 134 | E45SaCas9KKH9 | 45 | SaCas9 | + | TTCTGACAACAGTTTGCCG | CCCAA |
| | | | KKH | | CTG | T |
| 135 | E45SaCas9KKH10 | 45 | SaCas9 | + | TTGCCGCTGCCCAATGCCA | TGGAG |
| | | | KKH | | TCC | T |
| 136 | E45SaCas9KKH16 | 45 | SaCas9 | − | AAACAGCTGTCAGACAGAA | AGAGG |
| | | | KKH | | AAA | T |
| 137 | E45SaCas9KKH17 | 45 | SaCas9 | − | GAAGCCTGAATCTGCGGTG | GGAGG |
| | | | KKH | | GCA | T |
| 138 | E45SaCas9KKH18 | 45 | SaCas9 | − | GAAAAATTGGGAAGCCTGA | TGCGG |
| | | | KKH | | ATC | T |
| 139 | E45SaCas9KKH19 | 45 | SaCas9 | − | TCTACAGGAAAAATTGGGA | CTGAA |
| | | | KKH | | AGC | T |
| 140 | E45SaCas9KKH20 | 45 | SaCas9 | − | ACAGATGCCAGTATTCTAC | AAAAA |
| | | | KKH | | AGG | T |
| 141 | E45SaCas9KKH21 | 45 | SaCas9 | − | TCAGCAATCCTCAAAAACA | GCCAG |
| | | | KKH | | GAT | T |
| 142 | E45SaCas9KKH22 | 45 | SaCas9 | − | AATAATTCAGCAATCCTCA | ACAGA |
| | | | KKH | | AAA | T |
| 143 | E45SaCas9KKH23 | 45 | SaCas9 | − | GCAACTGGGGAAGAAATAA | AGCAA |
| | | | KKH | | TTC | T |
| 144 | E45SaCas9KKH24 | 45 | SaCas9 | − | CATTGAATGCAACTGGGGA | AATAA |
| | | | KKH | | AGA | T |
| 145 | E45SaCas9KKH25 | 45 | SaCas9 | − | GAACATTGAATGCAACTGG | AGAAA |
| | | | KKH | | GGA | T |
| 146 | E45SaCas9KKH26 | 45 | SaCas9 | − | GCGGCAAACTGTTGTCAGA | TTGAA |
| | | | KKH | | ACA | T |
| 147 | E45SaCas9KKH27 | 45 | SaCas9 | − | TTTTGGTATCTTACAGGAA | CAGGA |
| | | | KKH | | CTC | T |
| 148 | E50SaCas9KKH1 | 50 | SaCas9 | + | GGGATCCAGTATACTTACA | TCCAA |
| | | | KKH | | GGC | T |
| 149 | E50SaCas9KKH2 | 50 | SaCas9 | + | ATCCAGTATACTTACAGGC | AATAG |
| | | | KKH | | TCC | T |
| 150 | E50SaCas9KKH3 | 50 | SaCas9 | + | CAGTATACTTACAGGCTCC | AGTGG |
| | | | KKH | | AAT | T |
| 151 | E50SaCas9KKH4 | 50 | SaCas9 | + | ATACTTACAGGCTCCAATA | GTCAG |
| | | | KKH | | GTG | T |
| 152 | E50SaCas9KKH5 | 50 | SaCas9 | + | CCAATAGTGGTCAGTCCAG | CTAGG |
| | | | KKH | | GAG | T |
| 153 | E50SaCas9KKH6 | 50 | SaCas9 | + | AGGCTGCTTTGCCCTCAGC | TGAAG |
| | | | KKH | | TCT | T |
| 154 | E50SaCas9KKH7 | 50 | SaCas9 | + | TTTGCCCTCAGCTCTTGAA | AACGG |
| | | | KKH | | GTA | T |
| 155 | E50SaCas9KKH8 | 50 | SaCas9 | + | TTTACCGCCTTCCACTCAG | TCAGA |
| | | | KKH | | AGC | T |
| 156 | E50SaCas9KKH10 | 50 | SaCas9 | − | GGACTGACCACTATTGGAG | GTAAG |
| | | | KKH | | CCT | T |
| 157 | E50SaCas9KKH11 | 50 | SaCas9 | − | AAGATCTGAGCTCTGAGTG | GGCGG |
| | | | KKH | | GAA | T |
| 158 | E50SaCas9KKH12 | 50 | SaCas9 | − | AGGAAGTTAGAAGATCTGA | CTGAG |
| | | | KKH | | GCT | T |
| 159 | E50SaCas9KKH13 | 50 | SaCas9 | − | TTTTCTGTTAAAGAGGAAG | GAAGA |
| | | | KKH | | TTA | T |
| 924 | E53SL3.1A-Cv5 | 53 | SluCas9 | | CATTTGGATTGCATCTACT | |
| | | | | | GTA | |
| 925 | E53SL3.1C-Cv5 | 53 | SluCas9 | | CCTTTGGATTGCATCTACT | |
| | | | | | GTA | |
| 926 | E53S13.1G-Cv5 | 53 | SluCas9 | | CGTTTGGATTGCATCTACT | |
| | | | | | GTA | |
| 927 | E53S13.2A-Cv5 | 53 | SluCas9 | | CTATTGGATTGCATCTACT | |
| | | | | | GTA | |
| 928 | E53S13.2C-Cv5 | 53 | SluCas9 | | CTCTTGGATTGCATCTACT | |
| | | | | | GTA | |
| 929 | E53S13.2G-Cv5 | 53 | SluCas9 | | CTGTTGGATTGCATCTACT | |
| | | | | | GTA | |
| 930 | E53S13.3A-Cv5 | 53 | SluCas9 | | CTTATGGATTGCATCTACT | |
| | | | | | GTA | |
| 931 | E53S13.3C-Cv5 | 53 | SluCas9 | | CTTCTGGATTGCATCTACT | |
| | | | | | GTA | |
| 932 | E53S13.3G-Cv5 | 53 | SluCas9 | | CTTGTGGATTGCATCTACT | |
| | | | | | GTA | |
| 933 | E53S13.4A-Cv5 | 53 | SluCas9 | | CTTTAGGATTGCATCTACT | |
| | | | | | GTA | |
| 934 | E53S13.4C-Cv5 | 53 | SluCas9 | | CTTTCGGATTGCATCTACT | |
| | | | | | GTA | |
| 935 | E53S13.4G-Cv5 | 53 | SluCas9 | | CTTTGGGATTGCATCTACT | |
| | | | | | GTA | |
| 936 | E53S13.del2-Cv5 | 53 | SluCas9 | | TTTGGATTGCATCTACTGT | |
| | | | | | A | |
| 937 | E53S13.delT-Cv5 | 53 | SluCas9 | | CTTTGGATTGCATCTACTG | |
| | | | | | TA | |
| 938 | E53S13.delT + A.-Cv5 | 53 | SluCas9 | | ACTTTGGATTGCATCTACT | |
| | | | | | GTA | |
| 950 | E53SL3-21 | 53 | SluCas9 | | TTTTGGATTGCATCTACTG | |
| | | | | | TA | |
| 951 | E53SL3-20 | 53 | SluCas9 | | TTTGGATTGCATCTACTGT | |
| | | | | | A | |
| 952 | E53SL3-19 | 53 | SluCas9 | | TTGGATTGCATCTACTGTA | |
| 953 | E53SL3-18 | 53 | SluCas9 | | TGGATTGCATCTACTGTA | |
| 954 | E53SL3-17 | 53 | SluCas9 | | GGATTGCATCTACTGTA | |
| 955 | E53SL3-16 | 53 | SluCas9 | | GATTGCATCTACTGTA | |

### Compositions

In some embodiments, a composition is provided comprising one or more guide RNAs, or one or more nucleic acids encoding one or more guide RNAs, comprising or consisting of one or more guide sequence of Table 6 (SEQ ID NOs: 1-159 for SaCas9 and 200-292, 924-938, 950- and 955 for SluCas9). Table 6 provides the endonuclease associated with each guide sequence such that for each guide sequence described herein the type of endonuclease to be paired with the guide (for compositions) or used with the guide (for methods/uses) can be determined.

In some embodiments, a composition is provided comprising one or more guide RNAs, or one or more nucleic acids encoding one or more guide RNAs, wherein the guide RNA comprises at least 16, 17, 18, 19, or 20 contiguous nucleotides of a guide sequence of Table 6, or is at least 70%, at least 75%, at least 80%, at least 85%, or at least 90% identical to a guide sequence comprising at least 16, 17, 18, 19, or 20 nucleotides of a guide sequence of Table 6. In particular embodiments, a composition is provided comprising one or more guide RNAs, or one or more nucleic acids encoding one or more guide RNAs, wherein the guide RNA comprises at least 20 contiguous nucleotides of a guide sequence of Table 6, or is at least 70%, at least 75%, at least 80%, at least 85%, or at least 90% identical to guide sequence comprising at least 20 nucleotides of a guide sequence of Table 6. In other particular embodiments, a composition is provided comprising one or more guide RNAs, or one or more nucleic acids encoding one or more guide RNAs, wherein the guide RNA comprises no more than 16, no more than 17, no more than 18, no more than 19, no more than 20, no more than 21, or no more than 22 contiguous nucleotides of a guide sequence of Table 6, or is at least 70%, at least 75%, at least 80%, at least 85%, or at least 90% identical to guide sequence comprising no more than 16, no more than 17, no more than 18, no more than 19, no more than 20, no more than 21, or no more than 22 contiguous nucleotides of a guide sequence of Table 6.

In some embodiments, a composition is provided comprising two or more guide RNAs, or nucleic acid encoding two or more guide RNAs, wherein each guide RNA comprises a guide sequence of Table 6, or at least 16, 17, 18, 19, or 20 contiguous nucleotides of a guide sequence of Table 6, or is at least 70%, at least 75%, at least 80%, at least 85%, or at least 90% identical to a guide sequence comprising at least 16, 17, 18, 19, or 20 nucleotides of a guide sequence selected from Table 6. In particular embodiments, a composition is provided comprising two or more guide RNAs, or nucleic acid encoding two or more guide RNAs, wherein each guide RNA comprises at least 20 contiguous nucleotides of a guide sequence of Table 6, or is at least 70%, at least 75%, at least 80%, at least 85%, or at least 90% identical to a guide sequence comprising at least 20 nucleotides of a guide sequence selected from Table 6. In other particular embodiments, a composition is provided comprising two or more guide RNAs, or nucleic acid encoding two or more guide RNAs, wherein at least one of the two or more guide RNAs, optionally each guide RNA, comprises no more than 16, no more than 17, no more than 18, no more than 19, no more than 20, no more than 21, or no more than 22 contiguous nucleotides of a guide sequence of Table 6, or is at least 70%, at least 75%, at least 80%, at least 85%, or at least 90% identical to a guide sequence comprising no more than 16, no more than 17, no more than 18, no more than 19, no more than 20, no more than 21, or no more than 22 nucleotides of a guide sequence selected from Table 6.

In some embodiments, a composition is provided comprising a pair of guide RNAs, or nucleic acid encoding a pair of guide RNAs, wherein the pair of guide RNAs comprises sequences from any of the pairs disclosed in Tables 1B, 1D, 2B, 2D, 3B, 3D, 4B, 4D, 5B, and 5D. In some embodiments, a composition is provided comprising any of the pairs of guide RNA sequences disclosed in Tables 1B, 1D, 2B, 2D, 3B, 3D, 4B, 4D, 5B, and 5D.

In some embodiments, a composition is provided comprising one or more nucleic acid molecules, wherein at least one of the molecules comprises nucleic acid encoding: a) a SaCas9 or SluCas9; and b) a first and a second guide RNA comprising a first and a second guide sequence, wherein the first and second guide sequence are selected from any one of the guide sequence pairs of Tables 1B, 1D, 2B, 2D, 3B, 3D, 4B, 4D, 5B, and 5D.

In some embodiments, a composition is provided comprising two nucleic acid molecules, wherein at least one of the molecules comprises a nucleic acid encoding a first and a second guide RNA comprising a first and a second guide sequence, wherein the first and second guide sequence are selected from any one of the guide sequence pairs of Tables 1B, 1D, 2B, 2D, 3B, 3D, 4B, 4D, 5B, and 5D, optionally wherein the nucleic acid does not comprise a nucleic acid encoding an endonuclease.

In some embodiments, a composition is provided comprising a pair of guide RNAs, wherein the pair of guide RNAs comprises or consists of any one of the pairs of guide sequences of any one of Tables 1B or 1D for exon 44. In some embodiments, a composition is provided comprising a pair of guide RNAs, or nucleic acid encoding a pair of guide RNAs, wherein the pair of guide RNAs comprises or consists of any one of the pairs of guide sequences of any one of Tables 2B or 2D for exon 45. In some embodiments, a composition is provided comprising a pair of guide RNAs, wherein the pair of guide RNAs comprises or consists of any one of the pairs of guide sequences of any one of Tables 3B or 3D for exon 50. In some embodiments, a composition is provided comprising a pair of guide RNAs, wherein the pair of guide RNAs comprises or consists of any one of the pairs of guide sequences of any one of Tables 4B or 4D for exon 51. In some embodiments, a composition is provided comprising a pair of guide RNAs, wherein the pair of guide RNAs comprises or consists of any one of the pairs of guide sequences of any one of Tables 5B or 5D for exon 53. In some embodiments, a composition is provided comprising one or more nucleic acid molecules encoding a pair of guide RNAs, wherein the pair of guide RNAs comprises or consists of any one of the pairs of guide sequences of any one of Tables 1B or 1D for exon 44. In some embodiments, a composition is provided comprising one or more nucleic acid molecules encoding a pair of guide RNAs, wherein the pair of guide RNAs comprises or consists of any one of the pairs of guide sequences of any one of Tables 2B or 2D for exon 45. In some embodiments, a composition is provided comprising one or more nucleic acid molecules encoding a pair of guide RNAs, wherein the pair of guide RNAs comprises or consists of any one of the pairs of guide sequences of any one of Tables 3B or 3D for exon 50. In some embodiments, a composition is provided comprising one or more nucleic acid molecules encoding a pair of guide RNAs, wherein the pair of guide RNAs comprises or consists of any one of the pairs of guide sequences of any one of Tables 4B or 4D for exon 51. In some embodiments, a composition is provided comprising one or more nucleic acid molecules encoding a pair of guide RNAs, wherein the pair of guide RNAs comprises or consists of any one of the pairs of guide sequences of any one of Tables 5B or 5D for exon 53.

In some embodiments, a composition is provided comprising a pair of guide RNAs or a nucleic acid encoding a pair of guide RNAs, wherein the guide RNAs comprise or consist of any one of the following pairs of guide sequences:
a. For exon 44 (e.g., with SluCas9): SEQ ID NOs: 200 and 203; 200 and 204; 202 and 203; 202 and 205; 202 and 206; 202 and 207; 204 and 208; 204 and 205; 204 and 206; 204 and 207; or 204 and 208;
b. For exon 45 (e.g., with SluCas9), SEQ ID NOs: 223 and 230; or 224 and 212.
c. For exon 50 with SluCas9, SEQ ID NOs: 231 and 232; 231 and 234; 231 and 236; 231 and 237; 236 and 233; 236 and 235; 236 and 238; 236 and 240; or 236 and 241;
d. For exon 51 (e.g., with SaCas9), SEQ ID NOs: 28 and 57; 31 and 57; 31 and 46; 32 and 69; 33 and 57; 33 and 69; 34 and 57; 36 and 69; 37 and 69; 37 and 46; 39 and 46; 40 and 46; 46 and 35; 46 and 62; 46 and 65; 46 and 67; 47 and 57; 48 and 57; 49 and 57; 51 and 57; 53 and 46; 54 and 46; 55 and 46; 58 and 46; 59 and 46; 60 and 46; 61 and 69; or 69 and 30;
e. For exon 51 (e.g., with SluCas9), SEQ ID NOs: 243 and 252; 245 and 252; 252 and 253; 252 and 254; 252 and 256; 252 and 257; 252 and 258; 252 and 262; 252 and 264; 252 and 265; or 252 and 266;
f. For exon 53 (e.g., with SaCas9), SEQ ID NOs: 86 and 96; 87 and 96; 88 and 97; 89 and 96; 90 and 97; 92 and 77; 92 and 78; 92 and 96; 92 and 99; 93 and 98; 93 and 102; 94 and 100; 95 and 77; 95 and 78; 95 and 99; 96 and 100; 97 and 98; 97 and 102; 98 and 73; or 102 and 73; and
g. For exon 53 (e.g., with SluCas9), SEQ ID NOs: 278 and 290; 278 and 292; 281 and 291; 283 and 290; 283 and 292; 287 and 291; 272 and 290; 290 and 291; 272 and 292; 950 and 275; 951 and 275; 952 and 275; 953 and 275; 954 and 275; or 955 and 275.

In some embodiments, a composition is provided comprising: i) an SaCas9-KKH or a nucleic acid encoding a SaCas9-KKH, and ii) a first and a second guide RNA, or a nucleic acid encoding a first and a second guide RNA, wherein the first guide RNA comprises the nucleotide sequence of SEQ ID NO: 88, and wherein the second guide RNA comprises the nucleotide sequence of SEQ ID NO: 97. In some embodiments, a composition is provided comprising: i) a SluCas9 or a nucleic acid encoding a SluCas9, and ii) a first and a second guide RNA, or a nucleic acid encoding a first and a second guide RNA, wherein the first guide RNA comprises the nucleotide sequence of SEQ ID NO: 283, and wherein the second guide RNA comprises the nucleotide sequence of SEQ ID NO: 290. In some embodiments, a composition is provided comprising: i) a SluCas9 or a nucleic acid encoding a SluCas9, and ii) a first and a second guide RNA, or a nucleic acid encoding a first and a second guide RNA, wherein the first guide RNA comprises the nucleotide sequence of SEQ ID NO: 283, and wherein the second guide RNA comprises the nucleotide sequence of SEQ ID NO: 292.

In some embodiments, the disclosure provides for one or more nucleic acid molecules encoding a pair of guide RNAs comprising a first guide RNA and a second guide RNA, wherein the first guide RNA and the second guide RNA target exon 53. In some embodiments, the guide RNAs are for use with an SaCas9 endonuclease. In some embodiments, the one or more nucleic acid molecules also encode for an SaCas9 endonuclease. In some embodiments, the one or more nucleic acid molecules are in a composition further comprising an SaCas9 endonuclease. In some embodiments, the first guide RNA comprises the sequence of SEQ ID NOs: 86 and the second guide RNA comprises the sequence of SEQ ID NO: 96. In some embodiments, the first guide RNA comprises the sequence of SEQ ID NOs: 87 and the second guide RNA comprises the sequence of SEQ ID NO: 96. In some embodiments, the first guide RNA comprises the sequence of SEQ ID NOs: 88 and the second guide RNA comprises the sequence of SEQ ID NO: 97. In some embodiments, the first guide RNA comprises the sequence of SEQ ID NOs: 89 and the second guide RNA comprises the sequence of SEQ ID NO: 96. In some embodiments, the first guide RNA comprises the sequence of SEQ ID NOs: 90 and the second guide RNA comprises the sequence of SEQ ID NO: 97. In some embodiments, the first guide RNA comprises the sequence of SEQ ID NOs: 92 and the second guide RNA comprises the sequence of SEQ ID NO: 77. In some embodiments, the first guide RNA comprises the sequence of SEQ ID NOs: 92 and the second guide RNA comprises the sequence of SEQ ID NO: 78. In some embodiments, the first guide RNA comprises the sequence of SEQ ID NOs: 92 and the second guide RNA comprises the sequence of SEQ ID NO: 96. In some embodiments, the first guide RNA comprises the sequence of SEQ ID NOs: 92 and the second guide RNA comprises the sequence of SEQ ID NO: 99. In some embodiments, the first guide RNA comprises the sequence of SEQ ID NOs: 93 and the second guide RNA comprises the sequence of SEQ ID NO: 98. In some embodiments, the first guide RNA comprises the sequence of SEQ ID NOs: 93 and the second guide RNA comprises the sequence of SEQ ID NO: 102. In some embodiments, the first guide RNA comprises the sequence of SEQ ID NOs: 94 and the second guide RNA comprises the sequence of SEQ ID NO: 100. In some embodiments, the first guide RNA comprises the sequence of SEQ ID NOs: 95 and the second guide RNA comprises the sequence of SEQ ID NO: 77. In some embodiments, the first guide RNA comprises the sequence of SEQ ID NOs: 95 and the second guide RNA comprises the sequence of SEQ ID NO: 78. In some embodiments, the first guide RNA comprises the sequence of SEQ ID NOs: 95 and the second guide RNA comprises the sequence of SEQ ID NO: 99. In some embodiments, the first guide RNA comprises the sequence of SEQ ID NOs: 96 and the second guide RNA comprises the sequence of SEQ ID NO: 100. In some embodiments, the first guide RNA comprises the sequence of SEQ ID NOs: 97 and the second guide RNA comprises the sequence of SEQ ID NO: 98. In some embodiments, the first guide RNA comprises the sequence of SEQ ID NOs: 97 and the second guide RNA comprises the sequence of SEQ ID NO: 102. In some embodiments, the first guide RNA comprises the sequence of SEQ ID NOs: 98 and the second guide RNA comprises the sequence of SEQ ID NO: 73. In some embodiments, the first guide RNA comprises the sequence of SEQ ID NOs: 102 and the second guide RNA comprises the sequence of SEQ ID NO: 73. In some embodiments the guide RNAs each comprise a scaffold sequence comprising the sequence of SEQ ID NO: 504.

In some embodiments, the disclosure provides for one or more nucleic acid molecules encoding a pair of guide RNAs comprising a first guide RNA and a second guide RNA, wherein the first guide RNA and the second guide RNA target exon 53. In some embodiments, the guide RNAs are for use with an SluCas9 endonuclease. In some embodiments, the one or more nucleic acid molecules also encode for an SluCas9 endonuclease. In some embodiments, the one or more nucleic acid molecules are in a composition further comprising an SluCas9 endonuclease. In some embodiments, the first guide RNA comprises the sequence of SEQ ID NOs: 278 and the second guide RNA comprises the sequence of SEQ ID NO: 290. In some embodiments, the first guide RNA comprises the sequence of SEQ ID NOs: 278 and the second guide RNA comprises the sequence of SEQ ID NO: 292. In some embodiments, the first guide RNA comprises the sequence of SEQ ID NOs: 281 and the second guide RNA comprises the sequence of SEQ ID NO: 291. In some embodiments, the first guide RNA comprises the sequence of SEQ ID NOs: 283 and the second guide RNA comprises the sequence of SEQ ID NO: 290. In some embodiments, the first guide RNA comprises the sequence of SEQ ID NOs: 283 and the second guide RNA comprises the sequence of SEQ ID NO: 292. In some embodiments, the first guide RNA comprises the sequence of SEQ ID NOs: 287 and the second guide RNA comprises the sequence of SEQ ID NO: 291. In some embodiments, the first guide RNA comprises the sequence of SEQ ID NOs: 272 and the second guide RNA comprises the sequence of SEQ ID NO: 290. In some embodiments, the first guide RNA comprises the sequence of SEQ ID NOs: 291 and the second guide RNA comprises the sequence of SEQ ID NO: 290. In some embodiments, the first guide RNA comprises the sequence of SEQ ID NOs: 278 and the second guide RNA comprises the sequence of SEQ ID NO: 290. In some embodiments, the first guide RNA comprises the sequence of SEQ ID NOs: 272 and the second guide RNA comprises the sequence of SEQ ID NO: 292. In some embodiments the guide RNAs each comprise a scaffold sequence comprising the sequence of SEQ ID NO: 901.

In some embodiments, the disclosure provides for one or more nucleic acid molecules encoding a pair of guide RNAs comprising a first guide RNA and a second guide RNA, wherein the first guide RNA and the second guide RNA target exon 44. In some embodiments, the guide RNAs are for use with an SluCas9 endonuclease. In some embodiments, the one or more nucleic acid molecules also encode for an SluCas9 endonuclease. In some embodiments, the one or more nucleic acid molecules are in a composition further comprising an SluCas9 endonuclease. In some embodiments, the first guide RNA comprises the sequence of SEQ ID NOs: 200 and the second guide RNA comprises the sequence of SEQ ID NO: 203. In some embodiments, the first guide RNA comprises the sequence of SEQ ID NOs: 200 and the second guide RNA comprises the sequence of SEQ ID NO: 204. In some embodiments, the first guide RNA comprises the sequence of SEQ ID NOs: 202 and the second guide RNA comprises the sequence of SEQ ID NO: 203. In some embodiments, the first guide RNA comprises the sequence of SEQ ID NOs: 202 and the second guide RNA comprises the sequence of SEQ ID NO: 205. In some embodiments, the first guide RNA comprises the sequence of SEQ ID NOs: 202 and the second guide RNA comprises the sequence of SEQ ID NO: 206. In some embodiments, the first guide RNA comprises the sequence of SEQ ID NOs: 202 and the second guide RNA comprises the sequence of SEQ ID NO: 207. In some embodiments, the first guide RNA comprises the sequence of SEQ ID NOs: 204 and the second guide RNA comprises the sequence of SEQ ID NO: 208. In some embodiments, the first guide RNA comprises the sequence of SEQ ID NOs: 204 and the second guide RNA comprises the sequence of SEQ ID NO: 205. In some embodiments, the first guide RNA comprises the sequence of SEQ ID NOs: 204 and the second guide RNA comprises the sequence of SEQ ID NO: 206. In some embodiments, the first guide RNA comprises the sequence of SEQ ID NOs: 204 and the second guide RNA comprises the sequence of SEQ ID NO: 207. In some embodiments, the first guide RNA comprises the sequence of SEQ ID NOs: 204 and the second guide RNA comprises the sequence of SEQ ID NO: 208. In some embodiments the guide RNAs each comprise a scaffold sequence comprising the sequence of SEQ ID NO: 901.

In some embodiments, the disclosure provides for one or more nucleic acid molecules encoding a pair of guide RNAs comprising a first guide RNA and a second guide RNA, wherein the first guide RNA and the second guide RNA target exon 50. In some embodiments, the guide RNAs are for use with an SluCas9 endonuclease. In some embodiments, the one or more nucleic acid molecules also encode for an SluCas9 endonuclease. In some embodiments, the one or more nucleic acid molecules are in a composition further comprising an SluCas9 endonuclease. In some embodiments, the first guide RNA comprises the sequence of SEQ ID NOs: 231 and the second guide RNA comprises the sequence of SEQ ID NO: 232. In some embodiments, the first guide RNA comprises the sequence of SEQ ID NOs: 231 and the second guide RNA comprises the sequence of SEQ ID NO: 234. In some embodiments, the first guide RNA comprises the sequence of SEQ ID NOs: 231 and the second guide RNA comprises the sequence of SEQ ID NO: 236. In some embodiments, the first guide RNA comprises the sequence of SEQ ID NOs: 231 and the second guide RNA comprises the sequence of SEQ ID NO: 237. In some embodiments, the first guide RNA comprises the sequence of SEQ ID NOs: 236 and the second guide RNA comprises the sequence of SEQ ID NO: 233. In some embodiments, the first guide RNA comprises the sequence of SEQ ID NOs: 236 and the second guide RNA comprises the sequence of SEQ ID NO: 235. In some embodiments, the first guide RNA comprises the sequence of SEQ ID NOs: 236 and the second guide RNA comprises the sequence of SEQ ID NO: 238. In some embodiments, the first guide RNA comprises the sequence of SEQ ID NOs: 236 and the second guide RNA comprises the sequence of SEQ ID NO: 240. In some embodiments, the first guide RNA comprises the sequence of SEQ ID NOs: 236 and the second guide RNA comprises the sequence of SEQ ID NO: 241. In some embodiments the guide RNAs each comprise a scaffold sequence comprising the sequence of SEQ ID NO: 901.

In some embodiments, the composition further comprises an endonuclease or a nucleic acid encoding an endonuclease. An exemplary endonuclease for use with each of the guide RNAs or pairs of guide RNAs is provided herein, for example, in Table 6, at column “enzyme,” or in the “Guide ID” name as “Sa” for SaCas9, “SaCas9KKH” for SaCas9, or “SL” for SluCas9.

In some embodiments, a composition is provided comprising a single nucleic acid molecule comprising a nucleic acid encoding any of the guide RNAs disclosed herein, or any of the pairs of guide RNAs disclosed herein, and optionally a nucleic acid encoding an endonuclease.

In some embodiments, a composition is provided comprising at least two nucleic acid molecules comprising a nucleic acid encoding any of the guide RNAs disclosed herein, or any of the pairs of guide RNAs disclosed herein, and optionally a nucleic acid encoding an endonuclease, wherein at least one nucleic acid molecule does not comprise a nucleic acid encoding an endonuclease.

In some embodiments, a composition is provided comprising or consisting of a single nucleic acid molecule comprising: i) a nucleic acid encoding Staphylococcus aureus Cas9 (SaCas9) or Staphylococcus lugdunensis (SluCas9) and at least one, at least two, or at least three guide RNAs; or ii) a nucleic acid encoding Staphylococcus aureus Cas9 (SaCas9) or Staphylococcus lugdunensis (SluCas9) and from one to n guide RNAs, wherein n is no more than the maximum number of guide RNAs that can be expressed from said nucleic acid; or iii) a nucleic acid encoding Staphylococcus aureus Cas9 (SaCas9) or Staphylococcus lugdunensis (SluCas9) and one to three guide RNAs, wherein in each instance, the single nucleic acid molecule comprises a nucleic acid encoding at least one, at least two, or two guide sequence(s) of Table 6. In some embodiments, the single nucleic acid molecule comprises nucleic acid encoding any one of the following pairs of guide sequences:
a. For exon 44 (with e.g., SaCas9), SEQ ID NOs: 1 and 3; 110 and 120; 110 and 121; 110 and 122; 110 and 123; 110 and 124; 110 and 125; 111 and 112; 111 and 113; 111 and 114; 111 and 115; 111 and 116; 111 and 117; 111 and 118; 111 and 119; 111 and 120; 111 and 121; 111 and 122; 111 and 123; 111 and 124; 111 and 125; 112 and 113; 112 and 114; 112 and 115; 112 and 116; 112 and 117; 112 and 118; 112 and 119; 112 and 120; 112 and 121; 112 and 122; 112 and 123; 112 and 124; 112 and 125; 113 and 114; 113 and 115; 113 and 116; 113 and 117; 113 and 118; 113 and 119; 113 and 120; 113 and 121; 113 and 122; 113 and 123; 113 and 124; 113 and 125; 114 and 115; 114 and 116; 114 and 117; 114 and 118; 114 and 119; 114 and 120; 114 and 121; 114 and 122; 114 and 123; 114 and 124; 114 and 125; 115 and 116; 115 and 117; 115 and 118; 115 and 119; 115 and 120; 115 and 121; 115 and 122; 115 and 123; 115 and 124; 115 and 125; 116 and 117; 116 and 118; 116 and 119; 116 and 120; 116 and 121; 116 and 122; 116 and 123; 116 and 124; 116 and 125; 117 and 118; 117 and 119; 117 and 120; 117 and 121; 117 and 122; 117 and 123; 117 and 124; 117 and 125; 118 and 119; 118 and 120; 118 and 121; 118 and 122; 118 and 123; 118 and 124; 118 and 125; 119 and 120; 119 and 121; 119 and 122; 119 and 123; 119 and 124; 119 and 125; 120 and 121; 120 and 122; 120 and 123; 120 and 124; 120 and 125; 121 and 122; 121 and 123; 121 and 124; 121 and 125; 122 and 123; 122 and 124; 122 and 125; 123 and 124; 123 and 125; 124 and 125;
b. For exon 44 (with, e.g., SluCas9), SEQ ID NOs: 200 and 201; 200 and 202; 200 and 203; 200 and 204; 200 and 205; 200 and 206; 200 and 207; 200 and 208; 201 and 202; 201 and 203; 201 and 204; 201 and 205; 201 and 206; 201 and 207; 201 and 208; 202 and 203; 202 and 204; 202 and 205; 202 and 206; 202 and 207; 202 and 208; 203 and 204; 203 and 205; 203 and 206; 203 and 207; 203 and 208; 204 and 205; 204 and 206; 204 and 207; 204 and 208; 205 and 206; 205 and 207; 205 and 208; 206 and 207; 206 and 208; 207 and 208;
c. For exon 45 (with, e.g., SaCas9), SEQ ID NOs: 3 and 4; 3 and 5; 3 and 6; 3 and 7; 3 and 8; 3 and 9; 4 and 5; 4 and 6; 4 and 7; 4 and 8; 4 and 9; 5 and 6; 5 and 7; 5 and 8; 5 and 9; 6 and 7; 6 and 8; 6 and 9; 7 and 8; 7 and 9; 8 and 9; 126 and 127; 126 and 128; 126 and 129; 126 and 130; 126 and 131; 126 and 132; 126 and 133; 126 and 134; 126 and 135; 126 and 136; 126 and 137; 126 and 138; 126 and 139; 126 and 140; 126 and 141; 126 and 142; 126 and 143; 126 and 144; 126 and 145; 126 and 146; 126 and 147; 127 and 128; 127 and 129; 127 and 130; 127 and 131; 127 and 132; 127 and 133; 127 and 134; 127 and 135; 127 and 136; 127 and 137; 127 and 138; 127 and 139; 127 and 140; 127 and 141; 127 and 142; 127 and 143; 127 and 144; 127 and 145; 127 and 146; 127 and 147; 128 and 129; 128 and 130; 128 and 131; 128 and 132; 128 and 133; 128 and 134; 128 and 135; 128 and 136; 128 and 137; 128 and 138; 128 and 139; 128 and 140; 128 and 141; 128 and 142; 128 and 143; 128 and 144; 128 and 145; 128 and 146; 128 and 147; 129 and 130; 129 and 131; 129 and 132; 129 and 133; 129 and 134; 129 and 135; 129 and 136; 129 and 137; 129 and 138; 129 and 139; 129 and 140; 129 and 141; 129 and 142; 129 and 143; 129 and 144; 129 and 145; 129 and 146; 129 and 147; 130 and 131; 130 and 132; 130 and 133; 130 and 134; 130 and 135; 130 and 136; 130 and 137; 130 and 138; 130 and 139; 130 and 140; 130 and 141; 130 and 142; 130 and 143; 130 and 144; 130 and 145; 130 and 146; 130 and 147; 131 and 132; 131 and 133; 131 and 134; 131 and 135; 131 and 136; 131 and 137; 131 and 138; 131 and 139; 131 and 140; 131 and 141; 131 and 142; 131 and 143; 131 and 144; 131 and 145; 131 and 146; 131 and 147; 132 and 133; 132 and 134; 132 and 135; 132 and 136; 132 and 137; 132 and 138; 132 and 139; 132 and 140; 132 and 141; 132 and 142; 132 and 143; 132 and 144; 132 and 145; 132 and 146; 132 and 147; 133 and 134; 133 and 135; 133 and 136; 133 and 137; 133 and 138; 133 and 139; 133 and 140; 133 and 141; 133 and 142; 133 and 143; 133 and 144; 133 and 145; 133 and 146; 133 and 147; 134 and 135; 134 and 136; 134 and 137; 134 and 138; 134 and 139; 134 and 140; 134 and 141; 134 and 142; 134 and 143; 134 and 144; 134 and 145; 134 and 146; 134 and 147; 135 and 136; 135 and 137; 135 and 138; 135 and 139; 135 and 140; 135 and 141; 135 and 142; 135 and 143; 135 and 144; 135 and 145; 135 and 146; 135 and 147; 136 and 137; 136 and 138; 136 and 139; 136 and 140; 136 and 141; 136 and 142; 136 and 143; 136 and 144; 136 and 145; 136 and 146; 136 and 147; 137 and 138; 137 and 139; 137 and 140; 137 and 141; 137 and 142; 137 and 143; 137 and 144; 137 and 145; 137 and 146; 137 and 147; 138 and 139; 138 and 140; 138 and 141; 138 and 142; 138 and 143; 138 and 144; 138 and 145; 138 and 146; 138 and 147; 139 and 140; 139 and 141; 139 and 142; 139 and 143; 139 and 144; 139 and 145; 139 and 146; 139 and 147; 140 and 141; 140 and 142; 140 and 143; 140 and 144; 140 and 145; 140 and 146; 140 and 147; 141 and 142; 141 and 143; 141 and 144; 141 and 145; 141 and 146; 141 and 147; 142 and 143; 142 and 144; 142 and 145; 142 and 146; 142 and 147; 143 and 144; 143 and 145; 143 and 146; 143 and 147; 144 and 145; 144 and 146; 144 and 147; 145 and 146; 145 and 147; 146 and 147;
d. For exon 45 (with, e.g., SluCas9), SEQ ID NOs: 209 and 210; 209 and 211; 209 and 212; 209 and 213; 209 and 214; 209 and 215; 209 and 216; 209 and 217; 209 and 218; 209 and 219; 209 and 220; 209 and 221; 209 and 222; 209 and 223; 209 and 224; 209 and 225; 209 and 226; 209 and 227; 209 and 228; 209 and 229; 209 and 230; 210 and 211; 210 and 212; 210 and 213; 210 and 214; 210 and 215; 210 and 216; 210 and 217; 210 and 218; 210 and 219; 210 and 220; 210 and 221; 210 and 222; 210 and 223; 210 and 224; 210 and 225; 210 and 226; 210 and 227; 210 and 228; 210 and 229; 210 and 230; 211 and 212; 211 and 213; 211 and 214; 211 and 215; 211 and 216; 211 and 217; 211 and 218; 211 and 219; 211 and 220; 211 and 221; 211 and 222; 211 and 223; 211 and 224; 211 and 225; 211 and 226; 211 and 227; 211 and 228; 211 and 229; 211 and 230; 212 and 213; 212 and 214; 212 and 215; 212 and 216; 212 and 217; 212 and 218; 212 and 219; 212 and 220; 212 and 221; 212 and 222; 212 and 223; 212 and 224; 212 and 225; 212 and 226; 212 and 227; 212 and 228; 212 and 229; 212 and 230; 213 and 214; 213 and 215; 213 and 216; 213 and 217; 213 and 218; 213 and 219; 213 and 220; 213 and 221; 213 and 222; 213 and 223; 213 and 224; 213 and 225; 213 and 226; 213 and 227; 213 and 228; 213 and 229; 213 and 230; 214 and 215; 214 and 216; 214 and 217; 214 and 218; 214 and 219; 214 and 220; 214 and 221; 214 and 222; 214 and 223; 214 and 224; 214 and 225; 214 and 226; 214 and 227; 214 and 228; 214 and 229; 214 and 230; 215 and 216; 215 and 217; 215 and 218; 215 and 219; 215 and 220; 215 and 221; 215 and 222; 215 and 223; 215 and 224; 215 and 225; 215 and 226; 215 and 227; 215 and 228; 215 and 229; 215 and 230; 216 and 217; 216 and 218; 216 and 219; 216 and 220; 216 and 221; 216 and 222; 216 and 223; 216 and 224; 216 and 225; 216 and 226; 216 and 227; 216 and 228; 216 and 229; 216 and 230; 217 and 218; 217 and 219; 217 and 220; 217 and 221; 217 and 222; 217 and 223; 217 and 224; 217 and 225; 217 and 226; 217 and 227; 217 and 228; 217 and 229; 217 and 230; 218 and 219; 218 and 220; 218 and 221; 218 and 222; 218 and 223; 218 and 224; 218 and 225; 218 and 226; 218 and 227; 218 and 228; 218 and 229; 218 and 230; 219 and 220; 219 and 221; 219 and 222; 219 and 223; 219 and 224; 219 and 225; 219 and 226; 219 and 227; 219 and 228; 219 and 229; 219 and 230; 220 and 221; 220 and 222; 220 and 223; 220 and 224; 220 and 225; 220 and 226; 220 and 227; 220 and 228; 220 and 229; 220 and 230; 221 and 222; 221 and 223; 221 and 224; 221 and 225; 221 and 226; 221 and 227; 221 and 228; 221 and 229; 221 and 230; 222 and 223; 222 and 224; 222 and 225; 222 and 226; 222 and 227; 222 and 228; 222 and 229; 222 and 230; 223 and 224; 223 and 225; 223 and 226; 223 and 227; 223 and 228; 223 and 229; 223 and 230; 224 and 225; 224 and 226; 224 and 227; 224 and 228; 224 and 229; 224 and 230; 225 and 226; 225 and 227; 225 and 228; 225 and 229; 225 and 230; 226 and 227; 226 and 228; 226 and 229; 226 and 230; 227 and 228; 227 and 229; 227 and 230; 228 and 229; 228 and 230; 229 and 230;
e. For exon 50 (with, e.g., SaCas9), SEQ ID NOs: 148 and 149; 148 and 150; 148 and 151; 148 and 152; 148 and 153; 148 and 154; 148 and 155; 148 and 156; 148 and 157; 148 and 158; 148 and 159; 149 and 150; 149 and 151; 149 and 152; 149 and 153; 149 and 154; 149 and 155; 149 and 156; 149 and 157; 149 and 158; 149 and 159; 150 and 151; 150 and 152; 150 and 153; 150 and 154; 150 and 155; 150 and 156; 150 and 157; 150 and 158; 150 and 159; 151 and 152; 151 and 153; 151 and 154; 151 and 155; 151 and 156; 151 and 157; 151 and 158; 151 and 159; 152 and 153; 152 and 154; 152 and 155; 152 and 156; 152 and 157; 152 and 158; 152 and 159; 153 and 154; 153 and 155; 153 and 156; 153 and 157; 153 and 158; 153 and 159; 154 and 155; 154 and 156; 154 and 157; 154 and 158; 154 and 159; 155 and 156; 155 and 157; 155 and 158; 155 and 159; 156 and 157; 156 and 158; 156 and 159; 157 and 158; 157 and 159; 158 and 159;
f. For exon 50 (with, e.g., SluCas9), SEQ ID NOs: 231 and 232; 231 and 233; 231 and 234; 231 and 235; 231 and 236; 231 and 237; 231 and 238; 231 and 239; 231 and 240; 231 and 241; 231 and 242; 232 and 233; 232 and 234; 232 and 235; 232 and 236; 232 and 237; 232 and 238; 232 and 239; 232 and 240; 232 and 241; 232 and 242; 233 and 234; 233 and 235; 233 and 236; 233 and 237; 233 and 238; 233 and 239; 233 and 240; 233 and 241; 233 and 242; 234 and 235; 234 and 236; 234 and 237; 234 and 238; 234 and 239; 234 and 240; 234 and 241; 234 and 242; 235 and 236; 235 and 237; 235 and 238; 235 and 239; 235 and 240; 235 and 241; 235 and 242; 236 and 237; 236 and 238; 236 and 239; 236 and 240; 236 and 241; 236 and 242; 237 and 238; 237 and 239; 237 and 240; 237 and 241; 237 and 242; 238 and 239; 238 and 240; 238 and 241; 238 and 242; 239 and 240; 239 and 241; 239 and 242; 240 and 241; 240 and 242; 241 and 242;
g. For exon 51 (with, e.g., SaCas9), SEQ ID NOs: 11 and 12; 11 and 13; 11 and 14; 11 and 15; 12 and 13; 12 and 14; 12 and 15; 13 and 14; 13 and 15; 14 and 15; 27 and 28; 27 and 29; 27 and 30; 27 and 31; 27 and 32; 27 and 33; 27 and 34; 27 and 35; 27 and 36; 27 and 37; 27 and 38; 27 and 39; 27 and 40; 27 and 41; 27 and 42; 27 and 43; 27 and 44; 27 and 45; 27 and 46; 27 and 47; 27 and 48; 27 and 53; 27 and 54; 27 and 55; 27 and 56; 27 and 57; 27 and 58; 27 and 59; 27 and 60; 27 and 61; 27 and 62; 27 and 63; 27 and 64; 27 and 65; 27 and 66; 27 and 67; 27 and 68; 27 and 69; 28 and 29; 28 and 30; 28 and 31; 28 and 32; 28 and 33; 28 and 34; 28 and 35; 28 and 36; 28 and 37; 28 and 38; 28 and 39; 28 and 40; 28 and 41; 28 and 42; 28 and 43; 28 and 44; 28 and 45; 28 and 46; 28 and 47; 28 and 48; 28 and 53; 28 and 54; 28 and 55; 28 and 56; 28 and 57; 28 and 58; 28 and 59; 28 and 60; 28 and 61; 28 and 62; 28 and 63; 28 and 64; 28 and 65; 28 and 66; 28 and 67; 28 and 68; 28 and 69; 29 and 30; 29 and 31; 29 and 32; 29 and 33; 29 and 34; 29 and 35; 29 and 36; 29 and 37; 29 and 38; 29 and 39; 29 and 40; 29 and 41; 29 and 42; 29 and 43; 29 and 44; 29 and 45; 29 and 46; 29 and 47; 29 and 48; 29 and 53; 29 and 54; 29 and 55; 29 and 56; 29 and 57; 29 and 58; 29 and 59; 29 and 60; 29 and 61; 29 and 62; 29 and 63; 29 and 64; 29 and 65; 29 and 66; 29 and 67; 29 and 68; 29 and 69; 30 and 31; 30 and 32; 30 and 33; 30 and 34; 30 and 35; 30 and 36; 30 and 37; 30 and 38; 30 and 39; 30 and 40; 30 and 41; 30 and 42; 30 and 43; 30 and 44; 30 and 45; 30 and 46; 30 and 47; 30 and 48; 30 and 53; 30 and 54; 30 and 55; 30 and 56; 30 and 57; 30 and 58; 30 and 59; 30 and 60; 30 and 61; 30 and 62; 30 and 63; 30 and 64; 30 and 65; 30 and 66; 30 and 67; 30 and 68; 30 and 69; 31 and 32; 31 and 33; 31 and 34; 31 and 35; 31 and 36; 31 and 37; 31 and 38; 31 and 39; 31 and 40; 31 and 41; 31 and 42; 31 and 43; 31 and 44; 31 and 45; 31 and 46; 31 and 47; 31 and 48; 31 and 53; 31 and 54; 31 and 55; 31 and 56; 31 and 57; 31 and 58; 31 and 59; 31 and 60; 31 and 61; 31 and 62; 31 and 63; 31 and 64; 31 and 65; 31 and 66; 31 and 67; 31 and 68; 31 and 69; 32 and 33; 32 and 34; 32 and 35; 32 and 36; 32 and 37; 32 and 38; 32 and 39; 32 and 40; 32 and 41; 32 and 42; 32 and 43; 32 and 44; 32 and 45; 32 and 46; 32 and 47; 32 and 48; 32 and 53; 32 and 54; 32 and 55; 32 and 56; 32 and 57; 32 and 58; 32 and 59; 32 and 60; 32 and 61; 32 and 62; 32 and 63; 32 and 64; 32 and 65; 32 and 66; 32 and 67; 32 and 68; 32 and 69; 33 and 34; 33 and 35; 33 and 36; 33 and 37; 33 and 38; 33 and 39; 33 and 40; 33 and 41; 33 and 42; 33 and 43; 33 and 44; 33 and 45; 33 and 46; 33 and 47; 33 and 48; 33 and 53; 33 and 54; 33 and 55; 33 and 56; 33 and 57; 33 and 58; 33 and 59; 33 and 60; 33 and 61; 33 and 62; 33 and 63; 33 and 64; 33 and 65; 33 and 66; 33 and 67; 33 and 68; 33 and 69; 34 and 35; 34 and 36; 34 and 37; 34 and 38; 34 and 39; 34 and 40; 34 and 41; 34 and 42; 34 and 43; 34 and 44; 34 and 45; 34 and 46; 34 and 47; 34 and 48; 34 and 53; 34 and 54; 34 and 55; 34 and 56; 34 and 57; 34 and 58; 34 and 59; 34 and 60; 34 and 61; 34 and 62; 34 and 63; 34 and 64; 34 and 65; 34 and 66; 34 and 67; 34 and 68; 34 and 69; 35 and 36; 35 and 37; 35 and 38; 35 and 39; 35 and 40; 35 and 41; 35 and 42; 35 and 43; 35 and 44; 35 and 45; 35 and 46; 35 and 47; 35 and 48; 35 and 53; 35 and 54; 35 and 55; 35 and 56; 35 and 57; 35 and 58; 35 and 59; 35 and 60; 35 and 61; 35 and 62; 35 and 63; 35 and 64; 35 and 65; 35 and 66; 35 and 67; 35 and 68; 35 and 69; 36 and 37; 36 and 38; 36 and 39; 36 and 40; 36 and 41; 36 and 42; 36 and 43; 36 and 44; 36 and 45; 36 and 46; 36 and 47; 36 and 48; 36 and 53; 36 and 54; 36 and 55; 36 and 56; 36 and 57; 36 and 58; 36 and 59; 36 and 60; 36 and 61; 36 and 62; 36 and 63; 36 and 64; 36 and 65; 36 and 66; 36 and 67; 36 and 68; 36 and 69; 37 and 38; 37 and 39; 37 and 40; 37 and 41; 37 and 42; 37 and 43; 37 and 44; 37 and 45; 37 and 46; 37 and 47; 37 and 48; 37 and 53; 37 and 54; 37 and 55; 37 and 56; 37 and 57; 37 and 58; 37 and 59; 37 and 60; 37 and 61; 37 and 62; 37 and 63; 37 and 64; 37 and 65; 37 and 66; 37 and 67; 37 and 68; 37 and 69; 38 and 39; 38 and 40; 38 and 41; 38 and 42; 38 and 43; 38 and 44; 38 and 45; 38 and 46; 38 and 47; 38 and 48; 38 and 53; 38 and 54; 38 and 55; 38 and 56; 38 and 57; 38 and 58; 38 and 59; 38 and 60; 38 and 61; 38 and 62; 38 and 63; 38 and 64; 38 and 65; 38 and 66; 38 and 67; 38 and 68; 38 and 69; 39 and 40; 39 and 41; 39 and 42; 39 and 43; 39 and 44; 39 and 45; 39 and 46; 39 and 47; 39 and 48; 39 and 53; 39 and 54; 39 and 55; 39 and 56; 39 and 57; 39 and 58; 39 and 59; 39 and 60; 39 and 61; 39 and 62; 39 and 63; 39 and 64; 39 and 65; 39 and 66; 39 and 67; 39 and 68; 39 and 69; 40 and 41; 40 and 42; 40 and 43; 40 and 44; 40 and 45; 40 and 46; 40 and 47; 40 and 48; 40 and 53; 40 and 54; 40 and 55; 40 and 56; 40 and 57; 40 and 58; 40 and 59; 40 and 60; 40 and 61; 40 and 62; 40 and 63; 40 and 64; 40 and 65; 40 and 66; 40 and 67; 40 and 68; 40 and 69; 41 and 42; 41 and 43; 41 and 44; 41 and 45; 41 and 46; 41 and 47; 41 and 48; 41 and 53; 41 and 54; 41 and 55; 41 and 56; 41 and 57; 41 and 58; 41 and 59; 41 and 60; 41 and 61; 41 and 62; 41 and 63; 41 and 64; 41 and 65; 41 and 66; 41 and 67; 41 and 68; 41 and 69; 42 and 43; 42 and 44; 42 and 45; 42 and 46; 42 and 47; 42 and 48; 42 and 53; 42 and 54; 42 and 55; 42 and 56; 42 and 57; 42 and 58; 42 and 59; 42 and 60; 42 and 61; 42 and 62; 42 and 63; 42 and 64; 42 and 65; 42 and 66; 42 and 67; 42 and 68; 42 and 69; 43 and 44; 43 and 45; 43 and 46; 43 and 47; 43 and 48; 43 and 53; 43 and 54; 43 and 55; 43 and 56; 43 and 57; 43 and 58; 43 and 59; 43 and 60; 43 and 61; 43 and 62; 43 and 63; 43 and 64; 43 and 65; 43 and 66; 43 and 67; 43 and 68; 43 and 69; 44 and 45; 44 and 46; 44 and 47; 44 and 48; 44 and 53; 44 and 54; 44 and 55; 44 and 56; 44 and 57; 44 and 58; 44 and 59; 44 and 60; 44 and 61; 44 and 62; 44 and 63; 44 and 64; 44 and 65; 44 and 66; 44 and 67; 44 and 68; 44 and 69; 45 and 46; 45 and 47; 45 and 48; 45 and 53; 45 and 54; 45 and 55; 45 and 56; 45 and 57; 45 and 58; 45 and 59; 45 and 60; 45 and 61; 45 and 62; 45 and 63; 45 and 64; 45 and 65; 45 and 66; 45 and 67; 45 and 68; 45 and 69; 46 and 47; 46 and 48; 46 and 53; 46 and 54; 46 and 55; 46 and 56; 46 and 57; 46 and 58; 46 and 59; 46 and 60; 46 and 61; 46 and 62; 46 and 63; 46 and 64; 46 and 65; 46 and 66; 46 and 67; 46 and 68; 46 and 69; 47 and 48; 47 and 53; 47 and 54; 47 and 55; 47 and 56; 47 and 57; 47 and 58; 47 and 59; 47 and 60; 47 and 61; 47 and 62; 47 and 63; 47 and 64; 47 and 65; 47 and 66; 47 and 67; 47 and 68; 47 and 69; 48 and 53; 48 and 54; 48 and 55; 48 and 56; 48 and 57; 48 and 58; 48 and 59; 48 and 60; 48 and 61; 48 and 62; 48 and 63; 48 and 64; 48 and 65; 48 and 66; 48 and 67; 48 and 68; 48 and 69; 53 and 54; 53 and 55; 53 and 56; 53 and 57; 53 and 58; 53 and 59; 53 and 60; 53 and 61; 53 and 62; 53 and 63; 53 and 64; 53 and 65; 53 and 66; 53 and 67; 53 and 68; 53 and 69; 54 and 55; 54 and 56; 54 and 57; 54 and 58; 54 and 59; 54 and 60; 54 and 61; 54 and 62; 54 and 63; 54 and 64; 54 and 65; 54 and 66; 54 and 67; 54 and 68; 54 and 69; 55 and 56; 55 and 57; 55 and 58; 55 and 59; 55 and 60; 55 and 61; 55 and 62; 55 and 63; 55 and 64; 55 and 65; 55 and 66; 55 and 67; 55 and 68; 55 and 69; 56 and 57; 56 and 58; 56 and 59; 56 and 60; 56 and 61; 56 and 62; 56 and 63; 56 and 64; 56 and 65; 56 and 66; 56 and 67; 56 and 68; 56 and 69; 57 and 58; 57 and 59; 57 and 60; 57 and 61; 57 and 62; 57 and 63; 57 and 64; 57 and 65; 57 and 66; 57 and 67; 57 and 68; 57 and 69; 58 and 59; 58 and 60; 58 and 61; 58 and 62; 58 and 63; 58 and 64; 58 and 65; 58 and 66; 58 and 67; 58 and 68; 58 and 69; 59 and 60; 59 and 61; 59 and 62; 59 and 63; 59 and 64; 59 and 65; 59 and 66; 59 and 67; 59 and 68; 59 and 69; 60 and 61; 60 and 62; 60 and 63; 60 and 64; 60 and 65; 60 and 66; 60 and 67; 60 and 68; 60 and 69; 61 and 62; 61 and 63; 61 and 64; 61 and 65; 61 and 66; 61 and 67; 61 and 68; 61 and 69; 62 and 63; 62 and 64; 62 and 65; 62 and 66; 62 and 67; 62 and 68; 62 and 69; 63 and 64; 63 and 65; 63 and 66; 63 and 67; 63 and 68; 63 and 69; 64 and 65; 64 and 66; 64 and 67; 64 and 68; 64 and 69; 65 and 66; 65 and 67; 65 and 68; 65 and 69; 66 and 67; 66 and 68; 66 and 69; 67 and 68; 67 and 69; 68 and 69;
h. For exon 51 (with, e.g., SluCas9), SEQ ID NOs: 243 and 244; 243 and 245; 243 and 246; 243 and 247; 243 and 248; 243 and 249; 243 and 250; 243 and 251; 243 and 252; 243 and 253; 243 and 254; 243 and 255; 243 and 256; 243 and 257; 243 and 258; 243 and 259; 243 and 260; 243 and 261; 243 and 262; 243 and 263; 243 and 264; 243 and 265; 243 and 266; 243 and 267; 243 and 268; 243 and 269; 244 and 245; 244 and 246; 244 and 247; 244 and 248; 244 and 249; 244 and 250; 244 and 251; 244 and 252; 244 and 253; 244 and 254; 244 and 255; 244 and 256; 244 and 257; 244 and 258; 244 and 259; 244 and 260; 244 and 261; 244 and 262; 244 and 263; 244 and 264; 244 and 265; 244 and 266; 244 and 267; 244 and 268; 244 and 269; 245 and 246; 245 and 247; 245 and 248; 245 and 249; 245 and 250; 245 and 251; 245 and 252; 245 and 253; 245 and 254; 245 and 255; 245 and 256; 245 and 257; 245 and 258; 245 and 259; 245 and 260; 245 and 261; 245 and 262; 245 and 263; 245 and 264; 245 and 265; 245 and 266; 245 and 267; 245 and 268; 245 and 269; 246 and 247; 246 and 248; 246 and 249; 246 and 250; 246 and 251; 246 and 252; 246 and 253; 246 and 254; 246 and 255; 246 and 256; 246 and 257; 246 and 258; 246 and 259; 246 and 260; 246 and 261; 246 and 262; 246 and 263; 246 and 264; 246 and 265; 246 and 266; 246 and 267; 246 and 268; 246 and 269; 247 and 248; 247 and 249; 247 and 250; 247 and 251; 247 and 252; 247 and 253; 247 and 254; 247 and 255; 247 and 256; 247 and 257; 247 and 258; 247 and 259; 247 and 260; 247 and 261; 247 and 262; 247 and 263; 247 and 264; 247 and 265; 247 and 266; 247 and 267; 247 and 268; 247 and 269; 248 and 249; 248 and 250; 248 and 251; 248 and 252; 248 and 253; 248 and 254; 248 and 255; 248 and 256; 248 and 257; 248 and 258; 248 and 259; 248 and 260; 248 and 261; 248 and 262; 248 and 263; 248 and 264; 248 and 265; 248 and 266; 248 and 267; 248 and 268; 248 and 269; 249 and 250; 249 and 251; 249 and 252; 249 and 253; 249 and 254; 249 and 255; 249 and 256; 249 and 257; 249 and 258; 249 and 259; 249 and 260; 249 and 261; 249 and 262; 249 and 263; 249 and 264; 249 and 265; 249 and 266; 249 and 267; 249 and 268; 249 and 269; 250 and 251; 250 and 252; 250 and 253; 250 and 254; 250 and 255; 250 and 256; 250 and 257; 250 and 258; 250 and 259; 250 and 260; 250 and 261; 250 and 262; 250 and 263; 250 and 264; 250 and 265; 250 and 266; 250 and 267; 250 and 268; 250 and 269; 251 and 252; 251 and 253; 251 and 254; 251 and 255; 251 and 256; 251 and 257; 251 and 258; 251 and 259; 251 and 260; 251 and 261; 251 and 262; 251 and 263; 251 and 264; 251 and 265; 251 and 266; 251 and 267; 251 and 268; 251 and 269; 252 and 253; 252 and 254; 252 and 255; 252 and 256; 252 and 257; 252 and 258; 252 and 259; 252 and 260; 252 and 261; 252 and 262; 252 and 263; 252 and 264; 252 and 265; 252 and 266; 252 and 267; 252 and 268; 252 and 269; 253 and 254; 253 and 255; 253 and 256; 253 and 257; 253 and 258; 253 and 259; 253 and 260; 253 and 261; 253 and 262; 253 and 263; 253 and 264; 253 and 265; 253 and 266; 253 and 267; 253 and 268; 253 and 269; 254 and 255; 254 and 256; 254 and 257; 254 and 258; 254 and 259; 254 and 260; 254 and 261; 254 and 262; 254 and 263; 254 and 264; 254 and 265; 254 and 266; 254 and 267; 254 and 268; 254 and 269; 255 and 256; 255 and 257; 255 and 258; 255 and 259; 255 and 260; 255 and 261; 255 and 262; 255 and 263; 255 and 264; 255 and 265; 255 and 266; 255 and 267; 255 and 268; 255 and 269; 256 and 257; 256 and 258; 256 and 259; 256 and 260; 256 and 261; 256 and 262; 256 and 263; 256 and 264; 256 and 265; 256 and 266; 256 and 267; 256 and 268; 256 and 269; 257 and 258; 257 and 259; 257 and 260; 257 and 261; 257 and 262; 257 and 263; 257 and 264; 257 and 265; 257 and 266; 257 and 267; 257 and 268; 257 and 269; 258 and 259; 258 and 260; 258 and 261; 258 and 262; 258 and 263; 258 and 264; 258 and 265; 258 and 266; 258 and 267; 258 and 268; 258 and 269; 259 and 260; 259 and 261; 259 and 262; 259 and 263; 259 and 264; 259 and 265; 259 and 266; 259 and 267; 259 and 268; 259 and 269; 260 and 261; 260 and 262; 260 and 263; 260 and 264; 260 and 265; 260 and 266; 260 and 267; 260 and 268; 260 and 269; 261 and 262; 261 and 263; 261 and 264; 261 and 265; 261 and 266; 261 and 267; 261 and 268; 261 and 269; 262 and 263; 262 and 264; 262 and 265; 262 and 266; 262 and 267; 262 and 268; 262 and 269; 263 and 264; 263 and 265; 263 and 266; 263 and 267; 263 and 268; 263 and 269; 264 and 265; 264 and 266; 264 and 267; 264 and 268; 264 and 269; 265 and 266; 265 and 267; 265 and 268; 265 and 269; 266 and 267; 266 and 268; 266 and 269; 267 and 268; 267 and 269; 268 and 269;
i. For exon 53 (with, e.g., SaCas9), SEQ ID NOs: 16 and 17; 16 and 18; 16 and 19; 16 and 20; 16 and 21; 16 and 22; 16 and 23; 16 and 24; 16 and 25; 16 and 26; 17 and 18; 17 and 19; 17 and 20; 17 and 21; 17 and 22; 17 and 23; 17 and 24; 17 and 25; 17 and 26; 18 and 19; 18 and 20; 18 and 21; 18 and 22; 18 and 23; 18 and 24; 18 and 25; 18 and 26; 19 and 20; 19 and 21; 19 and 22; 19 and 23; 19 and 24; 19 and 25; 19 and 26; 20 and 21; 20 and 22; 20 and 23; 20 and 24; 20 and 25; 20 and 26; 21 and 22; 21 and 23; 21 and 24; 21 and 25; 21 and 26; 22 and 23; 22 and 24; 22 and 25; 22 and 26; 23 and 24; 23 and 25; 23 and 26; 24 and 25; 24 and 26; 25 and 26; 71 and 72; 71 and 73; 71 and 74; 71 and 75; 71 and 76; 71 and 77; 71 and 78; 71 and 84; 71 and 85; 71 and 86; 71 and 87; 71 and 88; 71 and 89; 71 and 90; 71 and 91; 71 and 92; 71 and 93; 71 and 94; 71 and 95; 71 and 96; 71 and 97; 71 and 98; 71 and 99; 71 and 100; 71 and 101; 71 and 102; 72 and 73; 72 and 74; 72 and 75; 72 and 76; 72 and 77; 72 and 78; 72 and 84; 72 and 85; 72 and 86; 72 and 87; 72 and 88; 72 and 89; 72 and 90; 72 and 91; 72 and 92; 72 and 93; 72 and 94; 72 and 95; 72 and 96; 72 and 97; 72 and 98; 72 and 99; 72 and 100; 72 and 101; 72 and 102; 73 and 74; 73 and 75; 73 and 76; 73 and 77; 73 and 78; 73 and 84; 73 and 85; 73 and 86; 73 and 87; 73 and 88; 73 and 89; 73 and 90; 73 and 91; 73 and 92; 73 and 93; 73 and 94; 73 and 95; 73 and 96; 73 and 97; 73 and 98; 73 and 99; 73 and 100; 73 and 101; 73 and 102; 74 and 75; 74 and 76; 74 and 77; 74 and 78; 74 and 84; 74 and 85; 74 and 86; 74 and 87; 74 and 88; 74 and 89; 74 and 90; 74 and 91; 74 and 92; 74 and 93; 74 and 94; 74 and 95; 74 and 96; 74 and 97; 74 and 98; 74 and 99; 74 and 100; 74 and 101; 74 and 102; 75 and 76; 75 and 77; 75 and 78; 75 and 84; 75 and 85; 75 and 86; 75 and 87; 75 and 88; 75 and 89; 75 and 90; 75 and 91; 75 and 92; 75 and 93; 75 and 94; 75 and 95; 75 and 96; 75 and 97; 75 and 98; 75 and 99; 75 and 100; 75 and 101; 75 and 102; 76 and 77; 76 and 78; 76 and 84; 76 and 85; 76 and 86; 76 and 87; 76 and 88; 76 and 89; 76 and 90; 76 and 91; 76 and 92; 76 and 93; 76 and 94; 76 and 95; 76 and 96; 76 and 97; 76 and 98; 76 and 99; 76 and 100; 76 and 101; 76 and 102; 77 and 78; 77 and 84; 77 and 85; 77 and 86; 77 and 87; 77 and 88; 77 and 89; 77 and 90; 77 and 91; 77 and 92; 77 and 93; 77 and 94; 77 and 95; 77 and 96; 77 and 97; 77 and 98; 77 and 99; 77 and 100; 77 and 101; 77 and 102; 78 and 84; 78 and 85; 78 and 86; 78 and 87; 78 and 88; 78 and 89; 78 and 90; 78 and 91; 78 and 92; 78 and 93; 78 and 94; 78 and 95; 78 and 96; 78 and 97; 78 and 98; 78 and 99; 78 and 100; 78 and 101; 78 and 102; 84 and 85; 84 and 86; 84 and 87; 84 and 88; 84 and 89; 84 and 90; 84 and 91; 84 and 92; 84 and 93; 84 and 94; 84 and 95; 84 and 96; 84 and 97; 84 and 98; 84 and 99; 84 and 100; 84 and 101; 84 and 102; 85 and 86; 85 and 87; 85 and 88; 85 and 89; 85 and 90; 85 and 91; 85 and 92; 85 and 93; 85 and 94; 85 and 95; 85 and 96; 85 and 97; 85 and 98; 85 and 99; 85 and 100; 85 and 101; 85 and 102; 86 and 87; 86 and 88; 86 and 89; 86 and 90; 86 and 91; 86 and 92; 86 and 93; 86 and 94; 86 and 95; 86 and 96; 86 and 97; 86 and 98; 86 and 99; 86 and 100; 86 and 101; 86 and 102; 87 and 88; 87 and 89; 87 and 90; 87 and 91; 87 and 92; 87 and 93; 87 and 94; 87 and 95; 87 and 96; 87 and 97; 87 and 98; 87 and 99; 87 and 100; 87 and 101; 87 and 102; 88 and 89; 88 and 90; 88 and 91; 88 and 92; 88 and 93; 88 and 94; 88 and 95; 88 and 96; 88 and 97; 88 and 98; 88 and 99; 88 and 100; 88 and 101; 88 and 102; 89 and 90; 89 and 91; 89 and 92; 89 and 93; 89 and 94; 89 and 95; 89 and 96; 89 and 97; 89 and 98; 89 and 99; 89 and 100; 89 and 101; 89 and 102; 90 and 91; 90 and 92; 90 and 93; 90 and 94; 90 and 95; 90 and 96; 90 and 97; 90 and 98; 90 and 99; 90 and 100; 90 and 101; 90 and 102; 91 and 92; 91 and 93; 91 and 94; 91 and 95; 91 and 96; 91 and 97; 91 and 98; 91 and 99; 91 and 100; 91 and 101; 91 and 102; 92 and 93; 92 and 94; 92 and 95; 92 and 96; 92 and 97; 92 and 98; 92 and 99; 92 and 100; 92 and 101; 92 and 102; 93 and 94; 93 and 95; 93 and 96; 93 and 97; 93 and 98; 93 and 99; 93 and 100; 93 and 101; 93 and 102; 94 and 95; 94 and 96; 94 and 97; 94 and 98; 94 and 99; 94 and 100; 94 and 101; 94 and 102; 95 and 96; 95 and 97; 95 and 98; 95 and 99; 95 and 100; 95 and 101; 95 and 102; 96 and 97; 96 and 98; 96 and 99; 96 and 100; 96 and 101; 96 and 102; 97 and 98; 97 and 99; 97 and 100; 97 and 101; 97 and 102; 98 and 99; 98 and 100; 98 and 101; 98 and 102; 99 and 100; 99 and 101; 99 and 102; 100 and 101; 100 and 102; 101 and 102;
j. For exon 53 (with, e.g., SluCas9), SEQ ID NOs: 270 and 271; 270 and 272; 270 and 273; 270 and 274; 270 and 275; 270 and 276; 270 and 277; 270 and 278; 270 and 279; 270 and 280; 270 and 281; 270 and 282; 270 and 283; 270 and 284; 270 and 285; 270 and 286; 270 and 287; 270 and 288; 270 and 289; 270 and 290; 270 and 291; 270 and 292; 271 and 272; 271 and 273; 271 and 274; 271 and 275; 271 and 276; 271 and 277; 271 and 278; 271 and 279; 271 and 280; 271 and 281; 271 and 282; 271 and 283; 271 and 284; 271 and 285; 271 and 286; 271 and 287; 271 and 288; 271 and 289; 271 and 290; 271 and 291; 271 and 292; 272 and 273; 272 and 274; 272 and 275; 272 and 276; 272 and 277; 272 and 278; 272 and 279; 272 and 280; 272 and 281; 272 and 282; 272 and 283; 272 and 284; 272 and 285; 272 and 286; 272 and 287; 272 and 288; 272 and 289; 272 and 290; 272 and 291; 272 and 292; 273 and 274; 273 and 275; 273 and 276; 273 and 277; 273 and 278; 273 and 279; 273 and 280; 273 and 281; 273 and 282; 273 and 283; 273 and 284; 273 and 285; 273 and 286; 273 and 287; 273 and 288; 273 and 289; 273 and 290; 273 and 291; 273 and 292; 274 and 275; 274 and 276; 274 and 277; 274 and 278; 274 and 279; 274 and 280; 274 and 281; 274 and 282; 274 and 283; 274 and 284; 274 and 285; 274 and 286; 274 and 287; 274 and 288; 274 and 289; 274 and 290; 274 and 291; 274 and 292; 275 and 276; 275 and 277; 275 and 278; 275 and 279; 275 and 280; 275 and 281; 275 and 282; 275 and 283; 275 and 284; 275 and 285; 275 and 286; 275 and 287; 275 and 288; 275 and 289; 275 and 290; 275 and 291; 275 and 292; 276 and 277; 276 and 278; 276 and 279; 276 and 280; 276 and 281; 276 and 282; 276 and 283; 276 and 284; 276 and 285; 276 and 286; 276 and 287; 276 and 288; 276 and 289; 276 and 290; 276 and 291; 276 and 292; 277 and 278; 277 and 279; 277 and 280; 277 and 281; 277 and 282; 277 and 283; 277 and 284; 277 and 285; 277 and 286; 277 and 287; 277 and 288; 277 and 289; 277 and 290; 277 and 291; 277 and 292; 278 and 279; 278 and 280; 278 and 281; 278 and 282; 278 and 283; 278 and 284; 278 and 285; 278 and 286; 278 and 287; 278 and 288; 278 and 289; 278 and 290; 278 and 291; 278 and 292; 279 and 280; 279 and 281; 279 and 282; 279 and 283; 279 and 284; 279 and 285; 279 and 286; 279 and 287; 279 and 288; 279 and 289; 279 and 290; 279 and 291; 279 and 292; 280 and 281; 280 and 282; 280 and 283; 280 and 284; 280 and 285; 280 and 286; 280 and 287; 280 and 288; 280 and 289; 280 and 290; 280 and 291; 280 and 292; 281 and 282; 281 and 283; 281 and 284; 281 and 285; 281 and 286; 281 and 287; 281 and 288; 281 and 289; 281 and 290; 281 and 291; 281 and 292; 282 and 283; 282 and 284; 282 and 285; 282 and 286; 282 and 287; 282 and 288; 282 and 289; 282 and 290; 282 and 291; 282 and 292; 283 and 284; 283 and 285; 283 and 286; 283 and 287; 283 and 288; 283 and 289; 283 and 290; 283 and 291; 283 and 292; 284 and 285; 284 and 286; 284 and 287; 284 and 288; 284 and 289; 284 and 290; 284 and 291; 284 and 292; 285 and 286; 285 and 287; 285 and 288; 285 and 289; 285 and 290; 285 and 291; 285 and 292; 286 and 287; 286 and 288; 286 and 289; 286 and 290; 286 and 291; 286 and 292; 287 and 288; 287 and 289; 287 and 290; 287 and 291; 287 and 292; 288 and 289; 288 and 290; 288 and 291; 288 and 292; 289 and 290; 289 and 291; 289 and 292; 290 and 291; 290 and 292; 291 and 292; 924 and 270; 924 and 272; 924 and 273; 924 and 274; 924 and 275; 924 and 276; 924 and 277; 924 and 278; 924 and 279; 924 and 280; 924 and 281; 924 and 282; 924 and 283; 924 and 284; 924 and 285; 924 and 286; 924 and 287; 924 and 288; 924 and 289; 924 and 290; 924 and 291; 924 and 292; 925 and 270; 925 and 272; 925 and 273; 925 and 274; 925 and 275; 925 and 276; 925 and 277; 925 and 278; 925 and 279; 925 and 280; 925 and 281; 925 and 282; 925 and 283; 925 and 284; 925 and 285; 925 and 286; 925 and 287; 925 and 288; 925 and 289; 925 and 290; 925 and 291; 925 and 292; 926 and 270; 926 and 272; 926 and 273; 926 and 274; 926 and 275; 926 and 276; 926 and 277; 926 and 278; 926 and 279; 926 and 280; 926 and 281; 926 and 282; 926 and 283; 926 and 284; 926 and 285; 926 and 286; 926 and 287; 926 and 288; 926 and 289; 926 and 290; 926 and 291; 926 and 292; 927 and 270; 927 and 272; 927 and 273; 927 and 274; 927 and 275; 927 and 276; 927 and 277; 927 and 278; 927 and 279; 927 and 280; 927 and 281; 927 and 282; 927 and 283; 927 and 284; 927 and 285; 927 and 286; 927 and 287; 927 and 288; 927 and 289; 927 and 290; 927 and 291; 927 and 292; 928 and 270; 928 and 272; 928 and 273; 928 and 274; 928 and 275; 928 and 276; 928 and 277; 928 and 278; 928 and 279; 928 and 280; 928 and 281; 928 and 282; 928 and 283; 928 and 284; 928 and 285; 928 and 286; 928 and 287; 928 and 288; 928 and 289; 928 and 290; 928 and 291; 928 and 292; 929 and 270; 929 and 272; 929 and 273; 929 and 274; 929 and 275; 929 and 276; 929 and 277; 929 and 278; 929 and 279; 929 and 280; 929 and 281; 929 and 282; 929 and 283; 929 and 284; 929 and 285; 929 and 286; 929 and 287; 929 and 288; 929 and 289; 929 and 290; 929 and 291; 929 and 292; 930 and 270; 930 and 272; 930 and 273; 930 and 274; 930 and 275; 930 and 276; 930 and 277; 930 and 278; 930 and 279; 930 and 280; 930 and 281; 930 and 282; 930 and 283; 930 and 284; 930 and 285; 930 and 286; 930 and 287; 930 and 288; 930 and 289; 930 and 290; 930 and 291; 930 and 292; 931 and 270; 931 and 272; 931 and 273; 931 and 274; 931 and 275; 931 and 276; 931 and 277; 931 and 278; 931 and 279; 931 and 280; 931 and 281; 931 and 282; 931 and 283; 931 and 284; 931 and 285; 931 and 286; 931 and 287; 931 and 288; 931 and 289; 931 and 290; 931 and 291; 931 and 292; 932 and 270; 932 and 272; 932 and 273; 932 and 274; 932 and 275; 932 and 276; 932 and 277; 932 and 278; 932 and 279; 932 and 280; 932 and 281; 932 and 282; 932 and 283; 932 and 284; 932 and 285; 932 and 286; 932 and 287; 932 and 288; 932 and 289; 932 and 290; 932 and 291; 932 and 292; 933 and 270; 933 and 272; 933 and 273; 933 and 274; 933 and 275; 933 and 276; 933 and 277; 933 and 278; 933 and 279; 933 and 280; 933 and 281; 933 and 282; 933 and 283; 933 and 284; 933 and 285; 933 and 286; 933 and 287; 933 and 288; 933 and 289; 933 and 290; 933 and 291; 933 and 292; 934 and 270; 934 and 272; 934 and 273; 934 and 274; 934 and 275; 934 and 276; 934 and 277; 934 and 278; 934 and 279; 934 and 280; 934 and 281; 934 and 282; 934 and 283; 934 and 284; 934 and 285; 934 and 286; 934 and 287; 934 and 288; 934 and 289; 934 and 290; 934 and 291; 934 and 292; 935 and 270; 935 and 272; 935 and 273; 935 and 274; 935 and 275; 935 and 276; 935 and 277; 935 and 278; 935 and 279; 935 and 280; 935 and 281; 935 and 282; 935 and 283; 935 and 284; 935 and 285; 935 and 286; 935 and 287; 935 and 288; 935 and 289; 935 and 290; 935 and 291; 935 and 292; 936 and 270; 936 and 272; 936 and 273; 936 and 274; 936 and 275; 936 and 276; 936 and 277; 936 and 278; 936 and 279; 936 and 280; 936 and 281; 936 and 282; 936 and 283; 936 and 284; 936 and 285; 936 and 286; 936 and 287; 936 and 288; 936 and 289; 936 and 290; 936 and 291; 936 and 292; 937 and 270; 937 and 272; 937 and 273; 937 and 274; 937 and 275; 937 and 276; 937 and 277; 937 and 278; 937 and 279; 937 and 280; 937 and 281; 937 and 282; 937 and 283; 937 and 284; 937 and 285; 937 and 286; 937 and 287; 937 and 288; 937 and 289; 937 and 290; 937 and 291; 937 and 292; 938 and 270; 938 and 272; 938 and 273; 938 and 274; 938 and 275; 938 and 276; 938 and 277; 938 and 278; 938 and 279; 938 and 280; 938 and 281; 938 and 282; 938 and 283; 938 and 284; 938 and 285; 938 and 286; 938 and 287; 938 and 288; 938 and 289; 938 and 290; 938 and 291; 938 and 292; 950 and 275; 951 and 275; 952 and 275; 953 and 275; 954 and 275; or 955 and 275;
k. For exon 44 (with, e.g., SluCas9), SEQ ID NOs: 200 and 203; 200 and 204; 202 and 203; 202 and 205; 202 and 206; 202 and 207; 204 and 208; 204 and 205; 204 and 206; 204 and 207; or 204 and 208;
l. For exon 45 (with, e.g., SluCas9), SEQ ID NOs: 223 and 230; or 224 and 212.
m. For exon 50 (with, e.g., SluCas9), SEQ ID NOs: 231 and 232; 231 and 234; 231 and 236; 231 and 237; 236 and 233; 236 and 235; 236 and 238; 236 and 240; or 236 and 241;
n. For exon 51 (with, e.g., SaCas9), SEQ ID NOs: 28 and 57; 31 and 57; 31 and 46; 32 and 69; 33 and 57; 33 and 69; 34 and 57; 36 and 69; 37 and 69; 37 and 46; 39 and 46; 40 and 46; 46 and 35; 46 and 62; 46 and 65; 46 and 67; 47 and 57; 48 and 57; 49 and 57; 51 and 57; 53 and 46; 54 and 46; 55 and 46; 58 and 46; 59 and 46; 60 and 46; 61 and 69; or 69 and 30;
o. For exon 51 (with, e.g., SluCas9), SEQ ID NOs: 243 and 252; 245 and 252; 252 and 253; 252 and 254; 252 and 256; 252 and 257; 252 and 258; 252 and 262; 252 and 264; 252 and 265; or 252 and 266;
p. For exon 53 (with, e.g., SaCas9), SEQ ID NOs: 86 and 96; 87 and 96; 88 and 97; 89 and 96; 90 and 97; 92 and 77; 92 and 78; 92 and 96; 92 and 99; 93 and 98; 93 and 102; 94 and 100; 95 and 77; 95 and 78; 95 and 99; 96 and 100; 97 and 98; 97 and 102; 98 and 73; or 102 and 73; and
q. For exon 53 (e.g., with SluCas9), SEQ ID NOs: 278 and 290; 278 and 292; 281 and 291; 283 and 290; 283 and 292; 287 and 291; 272 and 290; 290 and 291; or 272 and 292.

In some embodiments, a composition is provided comprising or consisting of at least two nucleic acid molecules comprising a first nucleic acid encoding Staphylococcus aureus Cas9 (SaCas9) or Staphylococcus lugdunensis (SluCas9) and i) a nucleic acid encoding at least one, at least two, or at least three guide RNAs; or ii) a nucleic acid encoding from one to n guide RNAs, wherein n is no more than the maximum number of guide RNAs that can be expressed from said nucleic acid; or iii) a nucleic acid encoding one to three guide RNAs; and a second nucleic acid that does not encode a SaCas9 or SluCas9, optionally wherein the second nucleic acid comprises any one of i) at least one, at least two, at least three, at least four, at least five, or at least six guide RNAs; or ii) from one to n guide RNAs, wherein n is no more than the maximum number of guide RNAs that can be expressed from said nucleic acid; or iii) from one to six guide RNAs, wherein in each instance, the guide RNAs comprises nucleic acid encoding at least one, at least two, or two guide sequence of Table 6. In some embodiments, at least one of the two nucleic acid molecules comprise nucleic acid encoding any one of the following pairs of guide sequences:
a. For exon 44 (e.g., with SaCas9), SEQ ID NOs: 1 and 3; 110 and 120; 110 and 121; 110 and 122; 110 and 123; 110 and 124; 110 and 125; 111 and 112; 111 and 113; 111 and 114; 111 and 115; 111 and 116; 111 and 117; 111 and 118; 111 and 119; 111 and 120; 111 and 121; 111 and 122; 111 and 123; 111 and 124; 111 and 125; 112 and 113; 112 and 114; 112 and 115; 112 and 116; 112 and 117; 112 and 118; 112 and 119; 112 and 120; 112 and 121; 112 and 122; 112 and 123; 112 and 124; 112 and 125; 113 and 114; 113 and 115; 113 and 116; 113 and 117; 113 and 118; 113 and 119; 113 and 120; 113 and 121; 113 and 122; 113 and 123; 113 and 124; 113 and 125; 114 and 115; 114 and 116; 114 and 117; 114 and 118; 114 and 119; 114 and 120; 114 and 121; 114 and 122; 114 and 123; 114 and 124; 114 and 125; 115 and 116; 115 and 117; 115 and 118; 115 and 119; 115 and 120; 115 and 121; 115 and 122; 115 and 123; 115 and 124; 115 and 125; 116 and 117; 116 and 118; 116 and 119; 116 and 120; 116 and 121; 116 and 122; 116 and 123; 116 and 124; 116 and 125; 117 and 118; 117 and 119; 117 and 120; 117 and 121; 117 and 122; 117 and 123; 117 and 124; 117 and 125; 118 and 119; 118 and 120; 118 and 121; 118 and 122; 118 and 123; 118 and 124; 118 and 125; 119 and 120; 119 and 121; 119 and 122; 119 and 123; 119 and 124; 119 and 125; 120 and 121; 120 and 122; 120 and 123; 120 and 124; 120 and 125; 121 and 122; 121 and 123; 121 and 124; 121 and 125; 122 and 123; 122 and 124; 122 and 125; 123 and 124; 123 and 125; 124 and 125;
b. For exon 44 (e.g., with SluCas9), SEQ ID NOs: 200 and 201; 200 and 202; 200 and 203; 200 and 204; 200 and 205; 200 and 206; 200 and 207; 200 and 208; 201 and 202; 201 and 203; 201 and 204; 201 and 205; 201 and 206; 201 and 207; 201 and 208; 202 and 203; 202 and 204; 202 and 205; 202 and 206; 202 and 207; 202 and 208; 203 and 204; 203 and 205; 203 and 206; 203 and 207; 203 and 208; 204 and 205; 204 and 206; 204 and 207; 204 and 208; 205 and 206; 205 and 207; 205 and 208; 206 and 207; 206 and 208; 207 and 208;
c. For exon 45 (e.g., with SaCas9), SEQ ID NOs: 3 and 4; 3 and 5; 3 and 6; 3 and 7; 3 and 8; 3 and 9; 4 and 5; 4 and 6; 4 and 7; 4 and 8; 4 and 9; 5 and 6; 5 and 7; 5 and 8; 5 and 9; 6 and 7; 6 and 8; 6 and 9; 7 and 8; 7 and 9; 8 and 9; 126 and 127; 126 and 128; 126 and 129; 126 and 130; 126 and 131; 126 and 132; 126 and 133; 126 and 134; 126 and 135; 126 and 136; 126 and 137; 126 and 138; 126 and 139; 126 and 140; 126 and 141; 126 and 142; 126 and 143; 126 and 144; 126 and 145; 126 and 146; 126 and 147; 127 and 128; 127 and 129; 127 and 130; 127 and 131; 127 and 132; 127 and 133; 127 and 134; 127 and 135; 127 and 136; 127 and 137; 127 and 138; 127 and 139; 127 and 140; 127 and 141; 127 and 142; 127 and 143; 127 and 144; 127 and 145; 127 and 146; 127 and 147; 128 and 129; 128 and 130; 128 and 131; 128 and 132; 128 and 133; 128 and 134; 128 and 135; 128 and 136; 128 and 137; 128 and 138; 128 and 139; 128 and 140; 128 and 141; 128 and 142; 128 and 143; 128 and 144; 128 and 145; 128 and 146; 128 and 147; 129 and 130; 129 and 131; 129 and 132; 129 and 133; 129 and 134; 129 and 135; 129 and 136; 129 and 137; 129 and 138; 129 and 139; 129 and 140; 129 and 141; 129 and 142; 129 and 143; 129 and 144; 129 and 145; 129 and 146; 129 and 147; 130 and 131; 130 and 132; 130 and 133; 130 and 134; 130 and 135; 130 and 136; 130 and 137; 130 and 138; 130 and 139; 130 and 140; 130 and 141; 130 and 142; 130 and 143; 130 and 144; 130 and 145; 130 and 146; 130 and 147; 131 and 132; 131 and 133; 131 and 134; 131 and 135; 131 and 136; 131 and 137; 131 and 138; 131 and 139; 131 and 140; 131 and 141; 131 and 142; 131 and 143; 131 and 144; 131 and 145; 131 and 146; 131 and 147; 132 and 133; 132 and 134; 132 and 135; 132 and 136; 132 and 137; 132 and 138; 132 and 139; 132 and 140; 132 and 141; 132 and 142; 132 and 143; 132 and 144; 132 and 145; 132 and 146; 132 and 147; 133 and 134; 133 and 135; 133 and 136; 133 and 137; 133 and 138; 133 and 139; 133 and 140; 133 and 141; 133 and 142; 133 and 143; 133 and 144; 133 and 145; 133 and 146; 133 and 147; 134 and 135; 134 and 136; 134 and 137; 134 and 138; 134 and 139; 134 and 140; 134 and 141; 134 and 142; 134 and 143; 134 and 144; 134 and 145; 134 and 146; 134 and 147; 135 and 136; 135 and 137; 135 and 138; 135 and 139; 135 and 140; 135 and 141; 135 and 142; 135 and 143; 135 and 144; 135 and 145; 135 and 146; 135 and 147; 136 and 137; 136 and 138; 136 and 139; 136 and 140; 136 and 141; 136 and 142; 136 and 143; 136 and 144; 136 and 145; 136 and 146; 136 and 147; 137 and 138; 137 and 139; 137 and 140; 137 and 141; 137 and 142; 137 and 143; 137 and 144; 137 and 145; 137 and 146; 137 and 147; 138 and 139; 138 and 140; 138 and 141; 138 and 142; 138 and 143; 138 and 144; 138 and 145; 138 and 146; 138 and 147; 139 and 140; 139 and 141; 139 and 142; 139 and 143; 139 and 144; 139 and 145; 139 and 146; 139 and 147; 140 and 141; 140 and 142; 140 and 143; 140 and 144; 140 and 145; 140 and 146; 140 and 147; 141 and 142; 141 and 143; 141 and 144; 141 and 145; 141 and 146; 141 and 147; 142 and 143; 142 and 144; 142 and 145; 142 and 146; 142 and 147; 143 and 144; 143 and 145; 143 and 146; 143 and 147; 144 and 145; 144 and 146; 144 and 147; 145 and 146; 145 and 147; 146 and 147;
d. For exon 45 (e.g., with SluCas9), SEQ ID NOs: 209 and 210; 209 and 211; 209 and 212; 209 and 213; 209 and 214; 209 and 215; 209 and 216; 209 and 217; 209 and 218; 209 and 219; 209 and 220; 209 and 221; 209 and 222; 209 and 223; 209 and 224; 209 and 225; 209 and 226; 209 and 227; 209 and 228; 209 and 229; 209 and 230; 210 and 211; 210 and 212; 210 and 213; 210 and 214; 210 and 215; 210 and 216; 210 and 217; 210 and 218; 210 and 219; 210 and 220; 210 and 221; 210 and 222; 210 and 223; 210 and 224; 210 and 225; 210 and 226; 210 and 227; 210 and 228; 210 and 229; 210 and 230; 211 and 212; 211 and 213; 211 and 214; 211 and 215; 211 and 216; 211 and 217; 211 and 218; 211 and 219; 211 and 220; 211 and 221; 211 and 222; 211 and 223; 211 and 224; 211 and 225; 211 and 226; 211 and 227; 211 and 228; 211 and 229; 211 and 230; 212 and 213; 212 and 214; 212 and 215; 212 and 216; 212 and 217; 212 and 218; 212 and 219; 212 and 220; 212 and 221; 212 and 222; 212 and 223; 212 and 224; 212 and 225; 212 and 226; 212 and 227; 212 and 228; 212 and 229; 212 and 230; 213 and 214; 213 and 215; 213 and 216; 213 and 217; 213 and 218; 213 and 219; 213 and 220; 213 and 221; 213 and 222; 213 and 223; 213 and 224; 213 and 225; 213 and 226; 213 and 227; 213 and 228; 213 and 229; 213 and 230; 214 and 215; 214 and 216; 214 and 217; 214 and 218; 214 and 219; 214 and 220; 214 and 221; 214 and 222; 214 and 223; 214 and 224; 214 and 225; 214 and 226; 214 and 227; 214 and 228; 214 and 229; 214 and 230; 215 and 216; 215 and 217; 215 and 218; 215 and 219; 215 and 220; 215 and 221; 215 and 222; 215 and 223; 215 and 224; 215 and 225; 215 and 226; 215 and 227; 215 and 228; 215 and 229; 215 and 230; 216 and 217; 216 and 218; 216 and 219; 216 and 220; 216 and 221; 216 and 222; 216 and 223; 216 and 224; 216 and 225; 216 and 226; 216 and 227; 216 and 228; 216 and 229; 216 and 230; 217 and 218; 217 and 219; 217 and 220; 217 and 221; 217 and 222; 217 and 223; 217 and 224; 217 and 225; 217 and 226; 217 and 227; 217 and 228; 217 and 229; 217 and 230; 218 and 219; 218 and 220; 218 and 221; 218 and 222; 218 and 223; 218 and 224; 218 and 225; 218 and 226; 218 and 227; 218 and 228; 218 and 229; 218 and 230; 219 and 220; 219 and 221; 219 and 222; 219 and 223; 219 and 224; 219 and 225; 219 and 226; 219 and 227; 219 and 228; 219 and 229; 219 and 230; 220 and 221; 220 and 222; 220 and 223; 220 and 224; 220 and 225; 220 and 226; 220 and 227; 220 and 228; 220 and 229; 220 and 230; 221 and 222; 221 and 223; 221 and 224; 221 and 225; 221 and 226; 221 and 227; 221 and 228; 221 and 229; 221 and 230; 222 and 223; 222 and 224; 222 and 225; 222 and 226; 222 and 227; 222 and 228; 222 and 229; 222 and 230; 223 and 224; 223 and 225; 223 and 226; 223 and 227; 223 and 228; 223 and 229; 223 and 230; 224 and 225; 224 and 226; 224 and 227; 224 and 228; 224 and 229; 224 and 230; 225 and 226; 225 and 227; 225 and 228; 225 and 229; 225 and 230; 226 and 227; 226 and 228; 226 and 229; 226 and 230; 227 and 228; 227 and 229; 227 and 230; 228 and 229; 228 and 230; 229 and 230;
e. For exon 50 (e.g., with SaCas9), SEQ ID NOs: 148 and 149; 148 and 150; 148 and 151; 148 and 152; 148 and 153; 148 and 154; 148 and 155; 148 and 156; 148 and 157; 148 and 158; 148 and 159; 149 and 150; 149 and 151; 149 and 152; 149 and 153; 149 and 154; 149 and 155; 149 and 156; 149 and 157; 149 and 158; 149 and 159; 150 and 151; 150 and 152; 150 and 153; 150 and 154; 150 and 155; 150 and 156; 150 and 157; 150 and 158; 150 and 159; 151 and 152; 151 and 153; 151 and 154; 151 and 155; 151 and 156; 151 and 157; 151 and 158; 151 and 159; 152 and 153; 152 and 154; 152 and 155; 152 and 156; 152 and 157; 152 and 158; 152 and 159; 153 and 154; 153 and 155; 153 and 156; 153 and 157; 153 and 158; 153 and 159; 154 and 155; 154 and 156; 154 and 157; 154 and 158; 154 and 159; 155 and 156; 155 and 157; 155 and 158; 155 and 159; 156 and 157; 156 and 158; 156 and 159; 157 and 158; 157 and 159; 158 and 159;
f. For exon 50 (e.g., with SluCas9), SEQ ID NOs: 231 and 232; 231 and 233; 231 and 234; 231 and 235; 231 and 236; 231 and 237; 231 and 238; 231 and 239; 231 and 240; 231 and 241; 231 and 242; 232 and 233; 232 and 234; 232 and 235; 232 and 236; 232 and 237; 232 and 238; 232 and 239; 232 and 240; 232 and 241; 232 and 242; 233 and 234; 233 and 235; 233 and 236; 233 and 237; 233 and 238; 233 and 239; 233 and 240; 233 and 241; 233 and 242; 234 and 235; 234 and 236; 234 and 237; 234 and 238; 234 and 239; 234 and 240; 234 and 241; 234 and 242; 235 and 236; 235 and 237; 235 and 238; 235 and 239; 235 and 240; 235 and 241; 235 and 242; 236 and 237; 236 and 238; 236 and 239; 236 and 240; 236 and 241; 236 and 242; 237 and 238; 237 and 239; 237 and 240; 237 and 241; 237 and 242; 238 and 239; 238 and 240; 238 and 241; 238 and 242; 239 and 240; 239 and 241; 239 and 242; 240 and 241; 240 and 242; 241 and 242;
g. For exon 51 (e.g., with SaCas9), SEQ ID NOs: 11 and 12; 11 and 13; 11 and 14; 11 and 15; 12 and 13; 12 and 14; 12 and 15; 13 and 14; 13 and 15; 14 and 15; 27 and 28; 27 and 29; 27 and 30; 27 and 31; 27 and 32; 27 and 33; 27 and 34; 27 and 35; 27 and 36; 27 and 37; 27 and 38; 27 and 39; 27 and 40; 27 and 41; 27 and 42; 27 and 43; 27 and 44; 27 and 45; 27 and 46; 27 and 47; 27 and 48; 27 and 53; 27 and 54; 27 and 55; 27 and 56; 27 and 57; 27 and 58; 27 and 59; 27 and 60; 27 and 61; 27 and 62; 27 and 63; 27 and 64; 27 and 65; 27 and 66; 27 and 67; 27 and 68; 27 and 69; 28 and 29; 28 and 30; 28 and 31; 28 and 32; 28 and 33; 28 and 34; 28 and 35; 28 and 36; 28 and 37; 28 and 38; 28 and 39; 28 and 40; 28 and 41; 28 and 42; 28 and 43; 28 and 44; 28 and 45; 28 and 46; 28 and 47; 28 and 48; 28 and 53; 28 and 54; 28 and 55; 28 and 56; 28 and 57; 28 and 58; 28 and 59; 28 and 60; 28 and 61; 28 and 62; 28 and 63; 28 and 64; 28 and 65; 28 and 66; 28 and 67; 28 and 68; 28 and 69; 29 and 30; 29 and 31; 29 and 32; 29 and 33; 29 and 34; 29 and 35; 29 and 36; 29 and 37; 29 and 38; 29 and 39; 29 and 40; 29 and 41; 29 and 42; 29 and 43; 29 and 44; 29 and 45; 29 and 46; 29 and 47; 29 and 48; 29 and 53; 29 and 54; 29 and 55; 29 and 56; 29 and 57; 29 and 58; 29 and 59; 29 and 60; 29 and 61; 29 and 62; 29 and 63; 29 and 64; 29 and 65; 29 and 66; 29 and 67; 29 and 68; 29 and 69; 30 and 31; 30 and 32; 30 and 33; 30 and 34; 30 and 35; 30 and 36; 30 and 37; 30 and 38; 30 and 39; 30 and 40; 30 and 41; 30 and 42; 30 and 43; 30 and 44; 30 and 45; 30 and 46; 30 and 47; 30 and 48; 30 and 53; 30 and 54; 30 and 55; 30 and 56; 30 and 57; 30 and 58; 30 and 59; 30 and 60; 30 and 61; 30 and 62; 30 and 63; 30 and 64; 30 and 65; 30 and 66; 30 and 67; 30 and 68; 30 and 69; 31 and 32; 31 and 33; 31 and 34; 31 and 35; 31 and 36; 31 and 37; 31 and 38; 31 and 39; 31 and 40; 31 and 41; 31 and 42; 31 and 43; 31 and 44; 31 and 45; 31 and 46; 31 and 47; 31 and 48; 31 and 53; 31 and 54; 31 and 55; 31 and 56; 31 and 57; 31 and 58; 31 and 59; 31 and 60; 31 and 61; 31 and 62; 31 and 63; 31 and 64; 31 and 65; 31 and 66; 31 and 67; 31 and 68; 31 and 69; 32 and 33; 32 and 34; 32 and 35; 32 and 36; 32 and 37; 32 and 38; 32 and 39; 32 and 40; 32 and 41; 32 and 42; 32 and 43; 32 and 44; 32 and 45; 32 and 46; 32 and 47; 32 and 48; 32 and 53; 32 and 54; 32 and 55; 32 and 56; 32 and 57; 32 and 58; 32 and 59; 32 and 60; 32 and 61; 32 and 62; 32 and 63; 32 and 64; 32 and 65; 32 and 66; 32 and 67; 32 and 68; 32 and 69; 33 and 34; 33 and 35; 33 and 36; 33 and 37; 33 and 38; 33 and 39; 33 and 40; 33 and 41; 33 and 42; 33 and 43; 33 and 44; 33 and 45; 33 and 46; 33 and 47; 33 and 48; 33 and 53; 33 and 54; 33 and 55; 33 and 56; 33 and 57; 33 and 58; 33 and 59; 33 and 60; 33 and 61; 33 and 62; 33 and 63; 33 and 64; 33 and 65; 33 and 66; 33 and 67; 33 and 68; 33 and 69; 34 and 35; 34 and 36; 34 and 37; 34 and 38; 34 and 39; 34 and 40; 34 and 41; 34 and 42; 34 and 43; 34 and 44; 34 and 45; 34 and 46; 34 and 47; 34 and 48; 34 and 53; 34 and 54; 34 and 55; 34 and 56; 34 and 57; 34 and 58; 34 and 59; 34 and 60; 34 and 61; 34 and 62; 34 and 63; 34 and 64; 34 and 65; 34 and 66; 34 and 67; 34 and 68; 34 and 69; 35 and 36; 35 and 37; 35 and 38; 35 and 39; 35 and 40; 35 and 41; 35 and 42; 35 and 43; 35 and 44; 35 and 45; 35 and 46; 35 and 47; 35 and 48; 35 and 53; 35 and 54; 35 and 55; 35 and 56; 35 and 57; 35 and 58; 35 and 59; 35 and 60; 35 and 61; 35 and 62; 35 and 63; 35 and 64; 35 and 65; 35 and 66; 35 and 67; 35 and 68; 35 and 69; 36 and 37; 36 and 38; 36 and 39; 36 and 40; 36 and 41; 36 and 42; 36 and 43; 36 and 44; 36 and 45; 36 and 46; 36 and 47; 36 and 48; 36 and 53; 36 and 54; 36 and 55; 36 and 56; 36 and 57; 36 and 58; 36 and 59; 36 and 60; 36 and 61; 36 and 62; 36 and 63; 36 and 64; 36 and 65; 36 and 66; 36 and 67; 36 and 68; 36 and 69; 37 and 38; 37 and 39; 37 and 40; 37 and 41; 37 and 42; 37 and 43; 37 and 44; 37 and 45; 37 and 46; 37 and 47; 37 and 48; 37 and 53; 37 and 54; 37 and 55; 37 and 56; 37 and 57; 37 and 58; 37 and 59; 37 and 60; 37 and 61; 37 and 62; 37 and 63; 37 and 64; 37 and 65; 37 and 66; 37 and 67; 37 and 68; 37 and 69; 38 and 39; 38 and 40; 38 and 41; 38 and 42; 38 and 43; 38 and 44; 38 and 45; 38 and 46; 38 and 47; 38 and 48; 38 and 53; 38 and 54; 38 and 55; 38 and 56; 38 and 57; 38 and 58; 38 and 59; 38 and 60; 38 and 61; 38 and 62; 38 and 63; 38 and 64; 38 and 65; 38 and 66; 38 and 67; 38 and 68; 38 and 69; 39 and 40; 39 and 41; 39 and 42; 39 and 43; 39 and 44; 39 and 45; 39 and 46; 39 and 47; 39 and 48; 39 and 53; 39 and 54; 39 and 55; 39 and 56; 39 and 57; 39 and 58; 39 and 59; 39 and 60; 39 and 61; 39 and 62; 39 and 63; 39 and 64; 39 and 65; 39 and 66; 39 and 67; 39 and 68; 39 and 69; 40 and 41; 40 and 42; 40 and 43; 40 and 44; 40 and 45; 40 and 46; 40 and 47; 40 and 48; 40 and 53; 40 and 54; 40 and 55; 40 and 56; 40 and 57; 40 and 58; 40 and 59; 40 and 60; 40 and 61; 40 and 62; 40 and 63; 40 and 64; 40 and 65; 40 and 66; 40 and 67; 40 and 68; 40 and 69; 41 and 42; 41 and 43; 41 and 44; 41 and 45; 41 and 46; 41 and 47; 41 and 48; 41 and 53; 41 and 54; 41 and 55; 41 and 56; 41 and 57; 41 and 58; 41 and 59; 41 and 60; 41 and 61; 41 and 62; 41 and 63; 41 and 64; 41 and 65; 41 and 66; 41 and 67; 41 and 68; 41 and 69; 42 and 43; 42 and 44; 42 and 45; 42 and 46; 42 and 47; 42 and 48; 42 and 53; 42 and 54; 42 and 55; 42 and 56; 42 and 57; 42 and 58; 42 and 59; 42 and 60; 42 and 61; 42 and 62; 42 and 63; 42 and 64; 42 and 65; 42 and 66; 42 and 67; 42 and 68; 42 and 69; 43 and 44; 43 and 45; 43 and 46; 43 and 47; 43 and 48; 43 and 53; 43 and 54; 43 and 55; 43 and 56; 43 and 57; 43 and 58; 43 and 59; 43 and 60; 43 and 61; 43 and 62; 43 and 63; 43 and 64; 43 and 65; 43 and 66; 43 and 67; 43 and 68; 43 and 69; 44 and 45; 44 and 46; 44 and 47; 44 and 48; 44 and 53; 44 and 54; 44 and 55; 44 and 56; 44 and 57; 44 and 58; 44 and 59; 44 and 60; 44 and 61; 44 and 62; 44 and 63; 44 and 64; 44 and 65; 44 and 66; 44 and 67; 44 and 68; 44 and 69; 45 and 46; 45 and 47; 45 and 48; 45 and 53; 45 and 54; 45 and 55; 45 and 56; 45 and 57; 45 and 58; 45 and 59; 45 and 60; 45 and 61; 45 and 62; 45 and 63; 45 and 64; 45 and 65; 45 and 66; 45 and 67; 45 and 68; 45 and 69; 46 and 47; 46 and 48; 46 and 53; 46 and 54; 46 and 55; 46 and 56; 46 and 57; 46 and 58; 46 and 59; 46 and 60; 46 and 61; 46 and 62; 46 and 63; 46 and 64; 46 and 65; 46 and 66; 46 and 67; 46 and 68; 46 and 69; 47 and 48; 47 and 53; 47 and 54; 47 and 55; 47 and 56; 47 and 57; 47 and 58; 47 and 59; 47 and 60; 47 and 61; 47 and 62; 47 and 63; 47 and 64; 47 and 65; 47 and 66; 47 and 67; 47 and 68; 47 and 69; 48 and 53; 48 and 54; 48 and 55; 48 and 56; 48 and 57; 48 and 58; 48 and 59; 48 and 60; 48 and 61; 48 and 62; 48 and 63; 48 and 64; 48 and 65; 48 and 66; 48 and 67; 48 and 68; 48 and 69; 53 and 54; 53 and 55; 53 and 56; 53 and 57; 53 and 58; 53 and 59; 53 and 60; 53 and 61; 53 and 62; 53 and 63; 53 and 64; 53 and 65; 53 and 66; 53 and 67; 53 and 68; 53 and 69; 54 and 55; 54 and 56; 54 and 57; 54 and 58; 54 and 59; 54 and 60; 54 and 61; 54 and 62; 54 and 63; 54 and 64; 54 and 65; 54 and 66; 54 and 67; 54 and 68; 54 and 69; 55 and 56; 55 and 57; 55 and 58; 55 and 59; 55 and 60; 55 and 61; 55 and 62; 55 and 63; 55 and 64; 55 and 65; 55 and 66; 55 and 67; 55 and 68; 55 and 69; 56 and 57; 56 and 58; 56 and 59; 56 and 60; 56 and 61; 56 and 62; 56 and 63; 56 and 64; 56 and 65; 56 and 66; 56 and 67; 56 and 68; 56 and 69; 57 and 58; 57 and 59; 57 and 60; 57 and 61; 57 and 62; 57 and 63; 57 and 64; 57 and 65; 57 and 66; 57 and 67; 57 and 68; 57 and 69; 58 and 59; 58 and 60; 58 and 61; 58 and 62; 58 and 63; 58 and 64; 58 and 65; 58 and 66; 58 and 67; 58 and 68; 58 and 69; 59 and 60; 59 and 61; 59 and 62; 59 and 63; 59 and 64; 59 and 65; 59 and 66; 59 and 67; 59 and 68; 59 and 69; 60 and 61; 60 and 62; 60 and 63; 60 and 64; 60 and 65; 60 and 66; 60 and 67; 60 and 68; 60 and 69; 61 and 62; 61 and 63; 61 and 64; 61 and 65; 61 and 66; 61 and 67; 61 and 68; 61 and 69; 62 and 63; 62 and 64; 62 and 65; 62 and 66; 62 and 67; 62 and 68; 62 and 69; 63 and 64; 63 and 65; 63 and 66; 63 and 67; 63 and 68; 63 and 69; 64 and 65; 64 and 66; 64 and 67; 64 and 68; 64 and 69; 65 and 66; 65 and 67; 65 and 68; 65 and 69; 66 and 67; 66 and 68; 66 and 69; 67 and 68; 67 and 69; 68 and 69;
h. For exon 51 (e.g., with SluCas9), SEQ ID NOs: 243 and 244; 243 and 245; 243 and 246; 243 and 247; 243 and 248; 243 and 249; 243 and 250; 243 and 251; 243 and 252; 243 and 253; 243 and 254; 243 and 255; 243 and 256; 243 and 257; 243 and 258; 243 and 259; 243 and 260; 243 and 261; 243 and 262; 243 and 263; 243 and 264; 243 and 265; 243 and 266; 243 and 267; 243 and 268; 243 and 269; 244 and 245; 244 and 246; 244 and 247; 244 and 248; 244 and 249; 244 and 250; 244 and 251; 244 and 252; 244 and 253; 244 and 254; 244 and 255; 244 and 256; 244 and 257; 244 and 258; 244 and 259; 244 and 260; 244 and 261; 244 and 262; 244 and 263; 244 and 264; 244 and 265; 244 and 266; 244 and 267; 244 and 268; 244 and 269; 245 and 246; 245 and 247; 245 and 248; 245 and 249; 245 and 250; 245 and 251; 245 and 252; 245 and 253; 245 and 254; 245 and 255; 245 and 256; 245 and 257; 245 and 258; 245 and 259; 245 and 260; 245 and 261; 245 and 262; 245 and 263; 245 and 264; 245 and 265; 245 and 266; 245 and 267; 245 and 268; 245 and 269; 246 and 247; 246 and 248; 246 and 249; 246 and 250; 246 and 251; 246 and 252; 246 and 253; 246 and 254; 246 and 255; 246 and 256; 246 and 257; 246 and 258; 246 and 259; 246 and 260; 246 and 261; 246 and 262; 246 and 263; 246 and 264; 246 and 265; 246 and 266; 246 and 267; 246 and 268; 246 and 269; 247 and 248; 247 and 249; 247 and 250; 247 and 251; 247 and 252; 247 and 253; 247 and 254; 247 and 255; 247 and 256; 247 and 257; 247 and 258; 247 and 259; 247 and 260; 247 and 261; 247 and 262; 247 and 263; 247 and 264; 247 and 265; 247 and 266; 247 and 267; 247 and 268; 247 and 269; 248 and 249; 248 and 250; 248 and 251; 248 and 252; 248 and 253; 248 and 254; 248 and 255; 248 and 256; 248 and 257; 248 and 258; 248 and 259; 248 and 260; 248 and 261; 248 and 262; 248 and 263; 248 and 264; 248 and 265; 248 and 266; 248 and 267; 248 and 268; 248 and 269; 249 and 250; 249 and 251; 249 and 252; 249 and 253; 249 and 254; 249 and 255; 249 and 256; 249 and 257; 249 and 258; 249 and 259; 249 and 260; 249 and 261; 249 and 262; 249 and 263; 249 and 264; 249 and 265; 249 and 266; 249 and 267; 249 and 268; 249 and 269; 250 and 251; 250 and 252; 250 and 253; 250 and 254; 250 and 255; 250 and 256; 250 and 257; 250 and 258; 250 and 259; 250 and 260; 250 and 261; 250 and 262; 250 and 263; 250 and 264; 250 and 265; 250 and 266; 250 and 267; 250 and 268; 250 and 269; 251 and 252; 251 and 253; 251 and 254; 251 and 255; 251 and 256; 251 and 257; 251 and 258; 251 and 259; 251 and 260; 251 and 261; 251 and 262; 251 and 263; 251 and 264; 251 and 265; 251 and 266; 251 and 267; 251 and 268; 251 and 269; 252 and 253; 252 and 254; 252 and 255; 252 and 256; 252 and 257; 252 and 258; 252 and 259; 252 and 260; 252 and 261; 252 and 262; 252 and 263; 252 and 264; 252 and 265; 252 and 266; 252 and 267; 252 and 268; 252 and 269; 253 and 254; 253 and 255; 253 and 256; 253 and 257; 253 and 258; 253 and 259; 253 and 260; 253 and 261; 253 and 262; 253 and 263; 253 and 264; 253 and 265; 253 and 266; 253 and 267; 253 and 268; 253 and 269; 254 and 255; 254 and 256; 254 and 257; 254 and 258; 254 and 259; 254 and 260; 254 and 261; 254 and 262; 254 and 263; 254 and 264; 254 and 265; 254 and 266; 254 and 267; 254 and 268; 254 and 269; 255 and 256; 255 and 257; 255 and 258; 255 and 259; 255 and 260; 255 and 261; 255 and 262; 255 and 263; 255 and 264; 255 and 265; 255 and 266; 255 and 267; 255 and 268; 255 and 269; 256 and 257; 256 and 258; 256 and 259; 256 and 260; 256 and 261; 256 and 262; 256 and 263; 256 and 264; 256 and 265; 256 and 266; 256 and 267; 256 and 268; 256 and 269; 257 and 258; 257 and 259; 257 and 260; 257 and 261; 257 and 262; 257 and 263; 257 and 264; 257 and 265; 257 and 266; 257 and 267; 257 and 268; 257 and 269; 258 and 259; 258 and 260; 258 and 261; 258 and 262; 258 and 263; 258 and 264; 258 and 265; 258 and 266; 258 and 267; 258 and 268; 258 and 269; 259 and 260; 259 and 261; 259 and 262; 259 and 263; 259 and 264; 259 and 265; 259 and 266; 259 and 267; 259 and 268; 259 and 269; 260 and 261; 260 and 262; 260 and 263; 260 and 264; 260 and 265; 260 and 266; 260 and 267; 260 and 268; 260 and 269; 261 and 262; 261 and 263; 261 and 264; 261 and 265; 261 and 266; 261 and 267; 261 and 268; 261 and 269; 262 and 263; 262 and 264; 262 and 265; 262 and 266; 262 and 267; 262 and 268; 262 and 269; 263 and 264; 263 and 265; 263 and 266; 263 and 267; 263 and 268; 263 and 269; 264 and 265; 264 and 266; 264 and 267; 264 and 268; 264 and 269; 265 and 266; 265 and 267; 265 and 268; 265 and 269; 266 and 267; 266 and 268; 266 and 269; 267 and 268; 267 and 269; 268 and 269;
i. For exon 53 (e.g., with SaCas9), SEQ ID NOs: 16 and 17; 16 and 18; 16 and 19; 16 and 20; 16 and 21; 16 and 22; 16 and 23; 16 and 24; 16 and 25; 16 and 26; 17 and 18; 17 and 19; 17 and 20; 17 and 21; 17 and 22; 17 and 23; 17 and 24; 17 and 25; 17 and 26; 18 and 19; 18 and 20; 18 and 21; 18 and 22; 18 and 23; 18 and 24; 18 and 25; 18 and 26; 19 and 20; 19 and 21; 19 and 22; 19 and 23; 19 and 24; 19 and 25; 19 and 26; 20 and 21; 20 and 22; 20 and 23; 20 and 24; 20 and 25; 20 and 26; 21 and 22; 21 and 23; 21 and 24; 21 and 25; 21 and 26; 22 and 23; 22 and 24; 22 and 25; 22 and 26; 23 and 24; 23 and 25; 23 and 26; 24 and 25; 24 and 26; 25 and 26; 71 and 72; 71 and 73; 71 and 74; 71 and 75; 71 and 76; 71 and 77; 71 and 78; 71 and 84; 71 and 85; 71 and 86; 71 and 87; 71 and 88; 71 and 89; 71 and 90; 71 and 91; 71 and 92; 71 and 93; 71 and 94; 71 and 95; 71 and 96; 71 and 97; 71 and 98; 71 and 99; 71 and 100; 71 and 101; 71 and 102; 72 and 73; 72 and 74; 72 and 75; 72 and 76; 72 and 77; 72 and 78; 72 and 84; 72 and 85; 72 and 86; 72 and 87; 72 and 88; 72 and 89; 72 and 90; 72 and 91; 72 and 92; 72 and 93; 72 and 94; 72 and 95; 72 and 96; 72 and 97; 72 and 98; 72 and 99; 72 and 100; 72 and 101; 72 and 102; 73 and 74; 73 and 75; 73 and 76; 73 and 77; 73 and 78; 73 and 84; 73 and 85; 73 and 86; 73 and 87; 73 and 88; 73 and 89; 73 and 90; 73 and 91; 73 and 92; 73 and 93; 73 and 94; 73 and 95; 73 and 96; 73 and 97; 73 and 98; 73 and 99; 73 and 100; 73 and 101; 73 and 102; 74 and 75; 74 and 76; 74 and 77; 74 and 78; 74 and 84; 74 and 85; 74 and 86; 74 and 87; 74 and 88; 74 and 89; 74 and 90; 74 and 91; 74 and 92; 74 and 93; 74 and 94; 74 and 95; 74 and 96; 74 and 97; 74 and 98; 74 and 99; 74 and 100; 74 and 101; 74 and 102; 75 and 76; 75 and 77; 75 and 78; 75 and 84; 75 and 85; 75 and 86; 75 and 87; 75 and 88; 75 and 89; 75 and 90; 75 and 91; 75 and 92; 75 and 93; 75 and 94; 75 and 95; 75 and 96; 75 and 97; 75 and 98; 75 and 99; 75 and 100; 75 and 101; 75 and 102; 76 and 77; 76 and 78; 76 and 84; 76 and 85; 76 and 86; 76 and 87; 76 and 88; 76 and 89; 76 and 90; 76 and 91; 76 and 92; 76 and 93; 76 and 94; 76 and 95; 76 and 96; 76 and 97; 76 and 98; 76 and 99; 76 and 100; 76 and 101; 76 and 102; 77 and 78; 77 and 84; 77 and 85; 77 and 86; 77 and 87; 77 and 88; 77 and 89; 77 and 90; 77 and 91; 77 and 92; 77 and 93; 77 and 94; 77 and 95; 77 and 96; 77 and 97; 77 and 98; 77 and 99; 77 and 100; 77 and 101; 77 and 102; 78 and 84; 78 and 85; 78 and 86; 78 and 87; 78 and 88; 78 and 89; 78 and 90; 78 and 91; 78 and 92; 78 and 93; 78 and 94; 78 and 95; 78 and 96; 78 and 97; 78 and 98; 78 and 99; 78 and 100; 78 and 101; 78 and 102; 84 and 85; 84 and 86; 84 and 87; 84 and 88; 84 and 89; 84 and 90; 84 and 91; 84 and 92; 84 and 93; 84 and 94; 84 and 95; 84 and 96; 84 and 97; 84 and 98; 84 and 99; 84 and 100; 84 and 101; 84 and 102; 85 and 86; 85 and 87; 85 and 88; 85 and 89; 85 and 90; 85 and 91; 85 and 92; 85 and 93; 85 and 94; 85 and 95; 85 and 96; 85 and 97; 85 and 98; 85 and 99; 85 and 100; 85 and 101; 85 and 102; 86 and 87; 86 and 88; 86 and 89; 86 and 90; 86 and 91; 86 and 92; 86 and 93; 86 and 94; 86 and 95; 86 and 96; 86 and 97; 86 and 98; 86 and 99; 86 and 100; 86 and 101; 86 and 102; 87 and 88; 87 and 89; 87 and 90; 87 and 91; 87 and 92; 87 and 93; 87 and 94; 87 and 95; 87 and 96; 87 and 97; 87 and 98; 87 and 99; 87 and 100; 87 and 101; 87 and 102; 88 and 89; 88 and 90; 88 and 91; 88 and 92; 88 and 93; 88 and 94; 88 and 95; 88 and 96; 88 and 97; 88 and 98; 88 and 99; 88 and 100; 88 and 101; 88 and 102; 89 and 90; 89 and 91; 89 and 92; 89 and 93; 89 and 94; 89 and 95; 89 and 96; 89 and 97; 89 and 98; 89 and 99; 89 and 100; 89 and 101; 89 and 102; 90 and 91; 90 and 92; 90 and 93; 90 and 94; 90 and 95; 90 and 96; 90 and 97; 90 and 98; 90 and 99; 90 and 100; 90 and 101; 90 and 102; 91 and 92; 91 and 93; 91 and 94; 91 and 95; 91 and 96; 91 and 97; 91 and 98; 91 and 99; 91 and 100; 91 and 101; 91 and 102; 92 and 93; 92 and 94; 92 and 95; 92 and 96; 92 and 97; 92 and 98; 92 and 99; 92 and 100; 92 and 101; 92 and 102; 93 and 94; 93 and 95; 93 and 96; 93 and 97; 93 and 98; 93 and 99; 93 and 100; 93 and 101; 93 and 102; 94 and 95; 94 and 96; 94 and 97; 94 and 98; 94 and 99; 94 and 100; 94 and 101; 94 and 102; 95 and 96; 95 and 97; 95 and 98; 95 and 99; 95 and 100; 95 and 101; 95 and 102; 96 and 97; 96 and 98; 96 and 99; 96 and 100; 96 and 101; 96 and 102; 97 and 98; 97 and 99; 97 and 100; 97 and 101; 97 and 102; 98 and 99; 98 and 100; 98 and 101; 98 and 102; 99 and 100; 99 and 101; 99 and 102; 100 and 101; 100 and 102; 101 and 102;
j. For exon 53 (e.g., with SluCas9), SEQ ID NOs: 270 and 271; 270 and 272; 270 and 273; 270 and 274; 270 and 275; 270 and 276; 270 and 277; 270 and 278; 270 and 279; 270 and 280; 270 and 281; 270 and 282; 270 and 283; 270 and 284; 270 and 285; 270 and 286; 270 and 287; 270 and 288; 270 and 289; 270 and 290; 270 and 291; 270 and 292; 271 and 272; 271 and 273; 271 and 274; 271 and 275; 271 and 276; 271 and 277; 271 and 278; 271 and 279; 271 and 280; 271 and 281; 271 and 282; 271 and 283; 271 and 284; 271 and 285; 271 and 286; 271 and 287; 271 and 288; 271 and 289; 271 and 290; 271 and 291; 271 and 292; 272 and 273; 272 and 274; 272 and 275; 272 and 276; 272 and 277; 272 and 278; 272 and 279; 272 and 280; 272 and 281; 272 and 282; 272 and 283; 272 and 284; 272 and 285; 272 and 286; 272 and 287; 272 and 288; 272 and 289; 272 and 290; 272 and 291; 272 and 292; 273 and 274; 273 and 275; 273 and 276; 273 and 277; 273 and 278; 273 and 279; 273 and 280; 273 and 281; 273 and 282; 273 and 283; 273 and 284; 273 and 285; 273 and 286; 273 and 287; 273 and 288; 273 and 289; 273 and 290; 273 and 291; 273 and 292; 274 and 275; 274 and 276; 274 and 277; 274 and 278; 274 and 279; 274 and 280; 274 and 281; 274 and 282; 274 and 283; 274 and 284; 274 and 285; 274 and 286; 274 and 287; 274 and 288; 274 and 289; 274 and 290; 274 and 291; 274 and 292; 275 and 276; 275 and 277; 275 and 278; 275 and 279; 275 and 280; 275 and 281; 275 and 282; 275 and 283; 275 and 284; 275 and 285; 275 and 286; 275 and 287; 275 and 288; 275 and 289; 275 and 290; 275 and 291; 275 and 292; 276 and 277; 276 and 278; 276 and 279; 276 and 280; 276 and 281; 276 and 282; 276 and 283; 276 and 284; 276 and 285; 276 and 286; 276 and 287; 276 and 288; 276 and 289; 276 and 290; 276 and 291; 276 and 292; 277 and 278; 277 and 279; 277 and 280; 277 and 281; 277 and 282; 277 and 283; 277 and 284; 277 and 285; 277 and 286; 277 and 287; 277 and 288; 277 and 289; 277 and 290; 277 and 291; 277 and 292; 278 and 279; 278 and 280; 278 and 281; 278 and 282; 278 and 283; 278 and 284; 278 and 285; 278 and 286; 278 and 287; 278 and 288; 278 and 289; 278 and 290; 278 and 291; 278 and 292; 279 and 280; 279 and 281; 279 and 282; 279 and 283; 279 and 284; 279 and 285; 279 and 286; 279 and 287; 279 and 288; 279 and 289; 279 and 290; 279 and 291; 279 and 292; 280 and 281; 280 and 282; 280 and 283; 280 and 284; 280 and 285; 280 and 286; 280 and 287; 280 and 288; 280 and 289; 280 and 290; 280 and 291; 280 and 292; 281 and 282; 281 and 283; 281 and 284; 281 and 285; 281 and 286; 281 and 287; 281 and 288; 281 and 289; 281 and 290; 281 and 291; 281 and 292; 282 and 283; 282 and 284; 282 and 285; 282 and 286; 282 and 287; 282 and 288; 282 and 289; 282 and 290; 282 and 291; 282 and 292; 283 and 284; 283 and 285; 283 and 286; 283 and 287; 283 and 288; 283 and 289; 283 and 290; 283 and 291; 283 and 292; 284 and 285; 284 and 286; 284 and 287; 284 and 288; 284 and 289; 284 and 290; 284 and 291; 284 and 292; 285 and 286; 285 and 287; 285 and 288; 285 and 289; 285 and 290; 285 and 291; 285 and 292; 286 and 287; 286 and 288; 286 and 289; 286 and 290; 286 and 291; 286 and 292; 287 and 288; 287 and 289; 287 and 290; 287 and 291; 287 and 292; 288 and 289; 288 and 290; 288 and 291; 288 and 292; 289 and 290; 289 and 291; 289 and 292; 290 and 291; 290 and 292; 291 and 292; 924 and 270; 924 and 272; 924 and 273; 924 and 274; 924 and 275; 924 and 276; 924 and 277; 924 and 278; 924 and 279; 924 and 280; 924 and 281; 924 and 282; 924 and 283; 924 and 284; 924 and 285; 924 and 286; 924 and 287; 924 and 288; 924 and 289; 924 and 290; 924 and 291; 924 and 292; 925 and 270; 925 and 272; 925 and 273; 925 and 274; 925 and 275; 925 and 276; 925 and 277; 925 and 278; 925 and 279; 925 and 280; 925 and 281; 925 and 282; 925 and 283; 925 and 284; 925 and 285; 925 and 286; 925 and 287; 925 and 288; 925 and 289; 925 and 290; 925 and 291; 925 and 292; 926 and 270; 926 and 272; 926 and 273; 926 and 274; 926 and 275; 926 and 276; 926 and 277; 926 and 278; 926 and 279; 926 and 280; 926 and 281; 926 and 282; 926 and 283; 926 and 284; 926 and 285; 926 and 286; 926 and 287; 926 and 288; 926 and 289; 926 and 290; 926 and 291; 926 and 292; 927 and 270; 927 and 272; 927 and 273; 927 and 274; 927 and 275; 927 and 276; 927 and 277; 927 and 278; 927 and 279; 927 and 280; 927 and 281; 927 and 282; 927 and 283; 927 and 284; 927 and 285; 927 and 286; 927 and 287; 927 and 288; 927 and 289; 927 and 290; 927 and 291; 927 and 292; 928 and 270; 928 and 272; 928 and 273; 928 and 274; 928 and 275; 928 and 276; 928 and 277; 928 and 278; 928 and 279; 928 and 280; 928 and 281; 928 and 282; 928 and 283; 928 and 284; 928 and 285; 928 and 286; 928 and 287; 928 and 288; 928 and 289; 928 and 290; 928 and 291; 928 and 292; 929 and 270; 929 and 272; 929 and 273; 929 and 274; 929 and 275; 929 and 276; 929 and 277; 929 and 278; 929 and 279; 929 and 280; 929 and 281; 929 and 282; 929 and 283; 929 and 284; 929 and 285; 929 and 286; 929 and 287; 929 and 288; 929 and 289; 929 and 290; 929 and 291; 929 and 292; 930 and 270; 930 and 272; 930 and 273; 930 and 274; 930 and 275; 930 and 276; 930 and 277; 930 and 278; 930 and 279; 930 and 280; 930 and 281; 930 and 282; 930 and 283; 930 and 284; 930 and 285; 930 and 286; 930 and 287; 930 and 288; 930 and 289; 930 and 290; 930 and 291; 930 and 292; 931 and 270; 931 and 272; 931 and 273; 931 and 274; 931 and 275; 931 and 276; 931 and 277; 931 and 278; 931 and 279; 931 and 280; 931 and 281; 931 and 282; 931 and 283; 931 and 284; 931 and 285; 931 and 286; 931 and 287; 931 and 288; 931 and 289; 931 and 290; 931 and 291; 931 and 292; 932 and 270; 932 and 272; 932 and 273; 932 and 274; 932 and 275; 932 and 276; 932 and 277; 932 and 278; 932 and 279; 932 and 280; 932 and 281; 932 and 282; 932 and 283; 932 and 284; 932 and 285; 932 and 286; 932 and 287; 932 and 288; 932 and 289; 932 and 290; 932 and 291; 932 and 292; 933 and 270; 933 and 272; 933 and 273; 933 and 274; 933 and 275; 933 and 276; 933 and 277; 933 and 278; 933 and 279; 933 and 280; 933 and 281; 933 and 282; 933 and 283; 933 and 284; 933 and 285; 933 and 286; 933 and 287; 933 and 288; 933 and 289; 933 and 290; 933 and 291; 933 and 292; 934 and 270; 934 and 272; 934 and 273; 934 and 274; 934 and 275; 934 and 276; 934 and 277; 934 and 278; 934 and 279; 934 and 280; 934 and 281; 934 and 282; 934 and 283; 934 and 284; 934 and 285; 934 and 286; 934 and 287; 934 and 288; 934 and 289; 934 and 290; 934 and 291; 934 and 292; 935 and 270; 935 and 272; 935 and 273; 935 and 274; 935 and 275; 935 and 276; 935 and 277; 935 and 278; 935 and 279; 935 and 280; 935 and 281; 935 and 282; 935 and 283; 935 and 284; 935 and 285; 935 and 286; 935 and 287; 935 and 288; 935 and 289; 935 and 290; 935 and 291; 935 and 292; 936 and 270; 936 and 272; 936 and 273; 936 and 274; 936 and 275; 936 and 276; 936 and 277; 936 and 278; 936 and 279; 936 and 280; 936 and 281; 936 and 282; 936 and 283; 936 and 284; 936 and 285; 936 and 286; 936 and 287; 936 and 288; 936 and 289; 936 and 290; 936 and 291; 936 and 292; 937 and 270; 937 and 272; 937 and 273; 937 and 274; 937 and 275; 937 and 276; 937 and 277; 937 and 278; 937 and 279; 937 and 280; 937 and 281; 937 and 282; 937 and 283; 937 and 284; 937 and 285; 937 and 286; 937 and 287; 937 and 288; 937 and 289; 937 and 290; 937 and 291; 937 and 292; 938 and 270; 938 and 272; 938 and 273; 938 and 274; 938 and 275; 938 and 276; 938 and 277; 938 and 278; 938 and 279; 938 and 280; 938 and 281; 938 and 282; 938 and 283; 938 and 284; 938 and 285; 938 and 286; 938 and 287; 938 and 288; 938 and 289; 938 and 290; 938 and 291; 938 and 292; 950 and 275; 951 and 275; 952 and 275; 953 and 275; 954 and 275; or 955 and 275;
k. For exon 44 (e.g., with SluCas9), SEQ ID NOs: 200 and 203; 200 and 204; 202 and 203; 202 and 205; 202 and 206; 202 and 207; 204 and 208; 204 and 205; 204 and 206; 204 and 207; or 204 and 208;
l. For exon 45 (e.g., with SluCas9), SEQ ID NOs: 223 and 230; or 224 and 212.
m. For exon 50 with SluCas9, SEQ ID NOs: 231 and 232; 231 and 234; 231 and 236; 231 and 237; 236 and 233; 236 and 235; 236 and 238; 236 and 240; or 236 and 241;
n. For exon 51 (e.g., with SaCas9), SEQ ID NOs: 28 and 57; 31 and 57; 31 and 46; 32 and 69; 33 and 57; 33 and 69; 34 and 57; 36 and 69; 37 and 69; 37 and 46; 39 and 46; 40 and 46; 46 and 35; 46 and 62; 46 and 65; 46 and 67; 47 and 57; 48 and 57; 49 and 57; 51 and 57; 53 and 46; 54 and 46; 55 and 46; 58 and 46; 59 and 46; 60 and 46; 61 and 69; or 69 and 30;
o. For exon 51 (e.g., with SluCas9), SEQ ID NOs: 243 and 252; 245 and 252; 252 and 253; 252 and 254; 252 and 256; 252 and 257; 252 and 258; 252 and 262; 252 and 264; 252 and 265; or 252 and 266;
p. For exon 53 (e.g., with SaCas9), SEQ ID NOs: 86 and 96; 87 and 96; 88 and 97; 89 and 96; 90 and 97; 92 and 77; 92 and 78; 92 and 96; 92 and 99; 93 and 98; 93 and 102; 94 and 100; 95 and 77; 95 and 78; 95 and 99; 96 and 100; 97 and 98; 97 and 102; 98 and 73; or 102 and 73; and
q. For exon 53 (e.g., with SluCas9), SEQ ID NOs: 278 and 290; 278 and 292; 281 and 291; 283 and 290; 283 and 292; 287 and 291; 272 and 290; 290 and 291; or 272 and 292.

The disclosure herein may reference a “first and a second spacer” or a “first and a second guide RNA, gRNA, or sgRNA” followed by one or more pairs of specific sequences. It should be noted that the order of the sequences in the pair is not intended to be restricted to the order in which they are presented/described. For example, the phrase “the first sgRNA and the second sgRNA comprise the sequences of SEQ ID NOs: 10 and 15” could mean that the first sgRNA comprises the sequence of SEQ ID NO: 10 and the second sgRNA sequence comprises the sequence of SEQ ID NO: 15, or this phrase could mean that the first sgRNA comprises the sequence of SEQ ID NO: 15 and the second sgRNA sequence comprises the sequence of SEQ ID NO: 10.

In some embodiments, the first and/or the second nucleic acid, if present, comprises at least two guide RNAs. In some embodiments, the first and/or the second nucleic acid, if present, comprises at least three guide RNAs. In some embodiments, the first and/or the second nucleic acid, if present, comprises at least four guide RNAs. In some embodiments, the first and/or the second nucleic acid, if present, comprises at least five guide RNAs. In some embodiments, the first and/or the second nucleic acid, if present, comprises at least six guide RNAs. In some embodiments, the first nucleic acid encodes an endonuclease and at least one, at least two, or at least three guide RNAs. In some embodiments, the first nucleic acid comprises an endonuclease and from one to n guide RNAs, wherein n is no more than the maximum number of guide RNAs that can be expressed from said nucleic acid. In some embodiments, the first nucleic acid encodes an endonuclease and from one to three guide RNAs. In some embodiments, the first nucleic acid comprises 1, 2, 3, 4, 5, or 6 guide RNAs, but does not encode an endonuclease.

In some embodiments, the second nucleic acid, if present, encodes at least one, at least two, at least three, at least four, at least five, or at least six guide RNAs. In some embodiments, the second nucleic acid, if present, encodes from one to n guide RNAs, wherein n is no more than the maximum number of guide RNAs that can be expressed from said nucleic acid In some embodiments, the second nucleic acid, if present, encodes from one to six guide RNAs. In some embodiments, the second nucleic acid, if present, encodes 2-10, 2-9, 2-8, 2-7, 2-6, 2-5, 2-4, or 2-3 guide RNAs. In some embodiments, the second nucleic acid, if present, encodes 2, 3, 4, 5, or 6 guide RNAs. In some embodiments, the second nucleic acid comprises 1, 2, 3, 4, 5, or 6 guide RNAs, but does not encode an endonuclease.

In some embodiments, the disclosure provides for a composition comprising at least two nucleic acid molecules, wherein the first nucleic acid comprises a guide RNA and the second nucleic acid comprises a guide RNA, wherein the guide RNA encoded by the first nucleic acid and the second nucleic acid are the same. In some embodiments, the disclosure provides for a composition comprising at least two nucleic acid molecules, wherein the first nucleic acid comprises a guide RNA and the second nucleic acid comprises a guide RNA, wherein the guide RNA encoded by the first nucleic acid and the second nucleic acid are different. In some embodiments, the disclosure provides for a composition comprising at least two nucleic acid molecules, wherein the first nucleic acid comprises a guide RNA and the second nucleic acid comprises a guide RNA, wherein at least one guide RNA binds to a target sequence within an exon in the DMD gene that is upstream of a premature stop codon, and wherein at least one guide RNA binds to a target sequence within an exon in the DMD gene that is downstream of a premature stop codon. In some embodiments, the disclosure provides for a composition comprising at least two nucleic acid molecules, wherein the first nucleic acid comprises a guide RNA and the second nucleic acid comprises a guide RNA, wherein the same guide RNA is encoded by the nucleic acid of the first and second nucleic acid molecule. In some embodiments, the disclosure provides for a composition comprising at least two nucleic acid molecules, wherein the first nucleic acid comprises a guide RNA and the second nucleic acid comprises a guide RNA, wherein the second nucleic acid molecule encodes a guide RNA that binds to the same target sequence as the guide RNA in the first nucleic acid molecule. In some embodiments, the disclosure provides for a composition comprising at least two nucleic acid molecules, wherein the second nucleic acid molecule encodes at least 2, at least 3, at least 4, at least 5, or at least 6 guide RNAs, wherein the guide RNAs in the second nucleic acid molecule bind to the same target sequence as the guide RNA in the first nucleic acid molecule.

In some embodiments, the disclosure provides for a composition comprising at least two guide RNAs, i) each guide RNA targets the same genomic target sequence; ii) each guide RNA targets a different target sequence; or iii) at least one guide RNA targets one sequence and at least one guide RNA targets a different sequence.

In some embodiments, the disclosure provides for a composition comprising a guide RNA that binds to an exon of the DMD gene, wherein the exon is selected from exon 44, 45, 50, 51, and 53.

In some embodiments, the disclosure provides for a composition comprising at least two guide RNAs that bind to an exon of the DMD gene, wherein at least one guide RNA binds to a target sequence within an exon in the DMD gene, and wherein at least one guide RNA binds to a different target sequence within the same exon in the DMD gene.

In some embodiments, the disclosure provides for a composition comprising at least two guide RNAs, wherein at least one guide RNA binds to a target sequence within an exon that is upstream of a premature stop codon, and wherein at least one guide RNA binds to a target sequence within an exon that is downstream of a premature stop codon.

In some embodiments, the disclosure provides for a composition comprising at least two guide RNAs, wherein at least one guide RNA binds to a target sequence within an exon in the DMD gene, and wherein at least one guide RNA binds to a different target sequence within the same exon in the DMD gene, wherein when expressed in vitro or in vivo in the presence of an appropriate endonuclease (e.g., SaCas9 or SluCas9), a portion of the exon is excised. In some embodiments, the disclosure provides for a composition comprising at least two guide RNAs, wherein once expressed in vitro or in vivo in the presence of an appropriate endonuclease (e.g., SaCas9 or SluCas9), the endonuclease excises a portion of the exon.

In some embodiments, the disclosure provides for a composition comprising at least two guide RNAs, wherein once expressed in vitro or in vivo in the presence of an appropriate endonuclease (e.g., SaCas9 or SluCas9), the endonuclease excises a portion of the exon, and wherein the portion of the exon remaining after excision are rejoined with a one nucleotide insertion. In some embodiments, the disclosure provides for a composition comprising at least two guide RNAs, wherein once expressed in vitro or in vivo in the presence of an appropriate endonuclease (e.g., SaCas9 or SluCas9), the endonuclease excises a portion of the exon, wherein the portion of the exon remaining after excision is rejoined without a nucleotide insertion.

In some embodiments, the disclosure provides for a composition comprising at least two guide RNAs, wherein once expressed in vitro or in vivo in the presence of an appropriate endonuclease (e.g., SaCas9 or SluCas9), the endonuclease excises a portion of a dystrophin gene (e.g., an exon). In some embodiments, the portions of the gene remaining after excision are rejoined with a one nucleotide insertion or a two nucleotide insertion. In some embodiments, the portions of the exon remaining after excision are rejoined without any nucleotide insertion. “Precise segmental deletion” or “precise deletion” means that the dual cut process takes out a specific deletion. This precise deletion can lead to exon refraining. In some embodiments, the size of the excised portion is between 5 and 250, 5 and 225, 5 and 200, 5 and 190, 5 and 180, 5 and 170, 5 and 160, 5 and 150, 5 and 125, 5 and 120, 5 and 115, 5 and 110, 5 and 100, 5 and 95, 5 and 90, 5 and 85, 5 and 80, 5 and 75, 5 and 70, 5 and 65, 5 and 60, 5 and 55, 5 and 50, 5 and 45, 5 and 40, 5 and 35, 5 and 30, 5 and 25, 5 and 20, 5 and 15, and 5-10 nucleotides. In some embodiments, the size of excised portion is between 20 and 250, 20 and 225, 20 and 200, 20 and 190, 20 and 180, 20 and 170, 20 and 160, 20 and 150, 20 and 125, 20 and 120, 20 and 115, 20 and 110, 20 and 100, 20 and 95, 20 and 90, 20 and 85, 20 and 80, 20 and 75, 20 and 70, 20 and 65, 20 and 60, 20 and 55, 20 and 50, 20 and 45, 20 and 40, 20 and 35, 20 and 30, and 20 and 25 nucleotides. In some embodiments, the size of excised portion is between 50 and 250, 50 and 225, 50 and 200, 50 and 190, 50 and 180, 50 and 170, 50 and 160, 50 and 150, 50 and 125, 50 and 120, 50 and 115, 50 and 110, and 50 and 100 nucleotides. In some embodiments, the size of excised portion of the exon is between 8 and 167 nucleotides.

In particular embodiments, if the target is exon 45, 51 or 53 of the dystrophin gene, then the precise deletion facilitates a “3n+1” edit of the dystrophin gene, wherein “n” is any negative whole number (e.g., any negative whole number between −10 and −75). In some embodiments, “n” is −10, −11, −12, −13, −14, −15, −16, −17, −18, −19, −20, −21, −22, −23, −24, −25, −26, −27, −28, −29, −30, −31, −32, −33, −34, −35, −36, −37, −38, −39, −40, −41, −42, −43, −44, −45, −46, −47, −48, −49, −50, −51, −52, −53, −54, −55, −56, −57, −58, −59, −60, −61, −62, −63, −64, −65, −66, −67, −68, −69, −70, −71, −72, −73, −74, −75, −76, −77, −78, −79, or −80. For example, if “n” is −10, a 3n+1 edit would be −30 nucleotides +1 nucleotide, i.e., −29 nucleotides, meaning that the actual excision product would be 29 nucleotides in length. With this in mind, in some embodiments, the actual excision product may be any of the following lengths: 26, 29, 32, 35, 38, 41, 44, 47, 50, 53, 56, 59, 62, 65, 68, 71, 74, 77, 80, 83, 86, 89, 92, 95, 98, 101, 104, 107, 110, 113, 116, 119, 122, 125, 128, 131, 134, 137, 140, 143, 146, 149, 152, 155, 158, 161, 164, 167, 170, 173, 176, 179, 182, 185, 188, 191, 194, 197, 200, 203, 206, 209, 212, 215, or 218 nucleotides in length. In particular embodiments, the excised portion is 92 nucleotides in length. In other embodiments, the excised portion is 188 nucleotides in length. In particular embodiments, the excised portion is 71 nucleotides in length. In other embodiments, the excised portion is 92 nucleotides in length. In other embodiments, the excised portion is 188 nucleotides in length. In other embodiments, the excised portion is 119 nucleotides in length.

In particular embodiments, if the target is exon 44 or 50 of the dystrophin gene, then the precise deletion facilitates a “3n+2” edit of the dystrophin gene, wherein “n” is any negative whole number (e.g., any negative whole number between −10 and −75). In some embodiments, “n” is −10, −11, −12, −13, −14, −15, −16, −17, −18, −19, −20, −21, −22, −23, −24, −25, −26, −27, −28, −29, −30, −31, −32, −33, −34, −35, −36, −37, −38, −39, −40, −41, −42, −43, −44, −45, −46, −47, −48, −49, −50, −51, −52, −53, −54, −55, −56, −57, −58, −59, −60, −61, −62, −63, −64, −65, −66, −67, −68, −69, −70, −71, −72, −73, −74, −75, −76, −77, −78, −79, or −80. For example, if “n” is −10, a 3n+2 edit would be −30 nucleotides+2 nucleotide, i.e., −28 nucleotides, meaning that the actual excision product would be 28 nucleotides in length. With this in mind, in some embodiments, the actual excision product may be any of the following lengths: 25, 28, 31, 34, 37, 40, 43, 46, 49, 52, 55, 58, 61, 64, 67, 70, 73, 76, 79, 82, 85, 88, 91, 94, 97, 100, 103, 106, 109, 112, 115, 118, 121, 124, 127, 130, 133, 136, 139, 142, 145, 148, 151, 154, 157, 160, 163, 166, 169, 172, 175, 178, 181, 184, 187, 190, 193, 196, 199, 202, 205, 208, 211, 214, or 217 nucleotides in length.

In some embodiments, the disclosure provides for a composition comprising at least two guide RNAs, wherein once expressed in vitro or in vivo in the presence of an appropriate endonuclease (e.g., SaCas9 or SluCas9), the endonuclease excises a portion of the exon, wherein the size of the excised portion of the exon is between 5, 6, 7, 8, 9, 10, 15, or 20 and 250 nucleotides in length. In some embodiments, the disclosure provides for a composition comprising at least two guide RNAs, wherein once expressed in vitro or in vivo in the presence of an appropriate endonuclease (e.g., SaCas9 or SluCas9), the endonuclease excises a portion of the exon, wherein the size of excised portion of the exon is between 150 and 250, 100 and 250, 5 and 250, 5 and 200, 5 and 150, 5 and 100, 5 and 75, 5 and 50, 5 and 25, 5 and 10, 20 and 250, 20 and 200, 20 and 150, 20 and 100, 20 and 75, 20 and 50, 20 and 25, 50 and 250, 50 and 200, 50 and 150, 50 and 100, and 50 and 75 nucleotides.

In some embodiments, the disclosure provides for a composition comprising at least two guide RNAs, wherein once expressed in vitro or in vivo in the presence of an appropriate endonuclease (e.g., SaCas9 or SluCas9), the endonuclease excises a portion of the exon, wherein the size of excised portion of the exon is between 8 and 167 nucleotides.

In some embodiments, a guide RNA and a Cas9 are encoded on a single nucleic acid molecule. In some embodiments, the single nucleic acid molecule comprises a nucleic acid encoding a guide RNA and a nucleic acid encoding a SaCas9 or SluCas9. In some embodiments, two guide RNAs and a Cas9 are encoded on a single nucleic acid molecule. In some embodiments, the single nucleic acid molecule comprises a nucleic acid encoding a first guide RNA, a nucleic acid encoding a second guide RNA, and a nucleic acid encoding a SaCas9 or SluCas9. In some embodiments, the spacer sequences of the first and second guide RNAs are identical. In some embodiments, the spacer sequences of the first and second guide RNAs are not identical.

In some embodiments, a single nucleic acid molecule comprises a nucleic acid encoding a Cas9 and a nucleic acid encoding two guide RNAs, wherein the nucleic acid molecule encodes no more than two guide RNAs

In some embodiments, the single nucleic acid molecule is a single vector or comprised in a single vector. In some embodiments, the single vector expresses the two guide RNAs and Cas9. In some embodiments, a pair of guide RNAs and a Cas9 are provided on a single vector. In some embodiments, the single vector comprises a nucleic acid encoding a pair of guide RNAs and a nucleic acid encoding a SaCas9 or SluCas9. In some embodiments, two guide RNAs and a Cas9 are encoded on a single vector. In some embodiments, the single vector comprises a nucleic acid encoding a first guide RNA, a nucleic acid encoding a second guide RNA, and a nucleic acid encoding a SaCas9 or SluCas9. In some embodiments, the spacer sequences of the first and second guide RNAs are identical. In some embodiments, the spacer sequences of the first and second guide RNAs are not identical.

In some embodiments, the first nucleic acid molecule encodes for a Cas9 molecule and also encodes for one or more copies of a first guide RNA and one or more copies of a second guide RNA. In some embodiments, the first nucleic acid molecule encodes for a Cas9 molecule, but does not encode for any guide RNAs. In some embodiments, the second nucleic acid molecule encodes for one or more copies of a first guide RNA and one or more copies of a second guide RNA, wherein the second nucleic acid molecule does not encode for a Cas9 molecule.

In some embodiments, the disclosure provides for a composition comprising one or more guide sequences, or comprises any of the nucleic acid molecules disclosed herein encoding for one or more guide sequences. In some embodiments, the guide sequence is SEQ ID NO: 1; In some embodiments, the guide sequence is SEQ ID NO: 2; In some embodiments, the guide sequence is SEQ ID NO: 3; In some embodiments, the guide sequence is SEQ ID NO: 4; In some embodiments, the guide sequence is SEQ ID NO: 5; In some embodiments, the guide sequence is SEQ ID NO: 6; In some embodiments, the guide sequence is SEQ ID NO: 7; In some embodiments, the guide sequence is SEQ ID NO: 8; In some embodiments, the guide sequence is SEQ ID NO: 9; In some embodiments, the guide sequence is SEQ ID NO: 10; In some embodiments, the guide sequence is SEQ ID NO: 11; In some embodiments, the guide sequence is SEQ ID NO: 12; In some embodiments, the guide sequence is SEQ ID NO: 13; In some embodiments, the guide sequence is SEQ ID NO: 14; In some embodiments, the guide sequence is SEQ ID NO: 15; In some embodiments, the guide sequence is SEQ ID NO: 16; In some embodiments, the guide sequence is SEQ ID NO: 17; In some embodiments, the guide sequence is SEQ ID NO: 18; In some embodiments, the guide sequence is SEQ ID NO: 19; In some embodiments, the guide sequence is SEQ ID NO: 20; In some embodiments, the guide sequence is SEQ ID NO: 21; In some embodiments, the guide sequence is SEQ ID NO: 22; In some embodiments, the guide sequence is SEQ ID NO: 23; In some embodiments, the guide sequence is SEQ ID NO: 24; In some embodiments, the guide sequence is SEQ ID NO: 25; In some embodiments, the guide sequence is SEQ ID NO: 26; In some embodiments, the guide sequence is SEQ ID NO: 27; In some embodiments, the guide sequence is SEQ ID NO: 28; In some embodiments, the guide sequence is SEQ ID NO: 29; In some embodiments, the guide sequence is SEQ ID NO: 30; In some embodiments, the guide sequence is SEQ ID NO: 31; In some embodiments, the guide sequence is SEQ ID NO: 32; In some embodiments, the guide sequence is SEQ ID NO: 33; In some embodiments, the guide sequence is SEQ ID NO: 34; In some embodiments, the guide sequence is SEQ ID NO: 35; In some embodiments, the guide sequence is SEQ ID NO: 36; In some embodiments, the guide sequence is SEQ ID NO: 37; In some embodiments, the guide sequence is SEQ ID NO: 38; In some embodiments, the guide sequence is SEQ ID NO: 39; In some embodiments, the guide sequence is SEQ ID NO: 40; In some embodiments, the guide sequence is SEQ ID NO: 41; In some embodiments, the guide sequence is SEQ ID NO: 42; In some embodiments, the guide sequence is SEQ ID NO: 43; In some embodiments, the guide sequence is SEQ ID NO: 44; In some embodiments, the guide sequence is SEQ ID NO: 45; In some embodiments, the guide sequence is SEQ ID NO: 46; In some embodiments, the guide sequence is SEQ ID NO: 47; In some embodiments, the guide sequence is SEQ ID NO: 48; In some embodiments, the guide sequence is SEQ ID NO: 49; In some embodiments, the guide sequence is SEQ ID NO: 50; In some embodiments, the guide sequence is SEQ ID NO: 51; In some embodiments, the guide sequence is SEQ ID NO: 52; In some embodiments, the guide sequence is SEQ ID NO: 53; In some embodiments, the guide sequence is SEQ ID NO: 54; In some embodiments, the guide sequence is SEQ ID NO: 55; In some embodiments, the guide sequence is SEQ ID NO: 56; In some embodiments, the guide sequence is SEQ ID NO: 57; In some embodiments, the guide sequence is SEQ ID NO: 58; In some embodiments, the guide sequence is SEQ ID NO: 59; In some embodiments, the guide sequence is SEQ ID NO: 60; In some embodiments, the guide sequence is SEQ ID NO: 61; In some embodiments, the guide sequence is SEQ ID NO: 62; In some embodiments, the guide sequence is SEQ ID NO: 63; In some embodiments, the guide sequence is SEQ ID NO: 64; In some embodiments, the guide sequence is SEQ ID NO: 65; In some embodiments, the guide sequence is SEQ ID NO: 66; In some embodiments, the guide sequence is SEQ ID NO: 67; In some embodiments, the guide sequence is SEQ ID NO: 68; In some embodiments, the guide sequence is SEQ ID NO: 69; In some embodiments, the guide sequence is SEQ ID NO: 70; In some embodiments, the guide sequence is SEQ ID NO: 71; In some embodiments, the guide sequence is SEQ ID NO: 72; In some embodiments, the guide sequence is SEQ ID NO: 73; In some embodiments, the guide sequence is SEQ ID NO: 74; In some embodiments, the guide sequence is SEQ ID NO: 75; In some embodiments, the guide sequence is SEQ ID NO: 76; In some embodiments, the guide sequence is SEQ ID NO: 77; In some embodiments, the guide sequence is SEQ ID NO: 78; In some embodiments, the guide sequence is SEQ ID NO: 79; In some embodiments, the guide sequence is SEQ ID NO: 80; In some embodiments, the guide sequence is SEQ ID NO: 81; In some embodiments, the guide sequence is SEQ ID NO: 82; In some embodiments, the guide sequence is SEQ ID NO: 83; In some embodiments, the guide sequence is SEQ ID NO: 84; In some embodiments, the guide sequence is SEQ ID NO: 85; In some embodiments, the guide sequence is SEQ ID NO: 86; In some embodiments, the guide sequence is SEQ ID NO: 87; In some embodiments, the guide sequence is SEQ ID NO: 88; In some embodiments, the guide sequence is SEQ ID NO: 89; In some embodiments, the guide sequence is SEQ ID NO: 90; In some embodiments, the guide sequence is SEQ ID NO: 91; In some embodiments, the guide sequence is SEQ ID NO: 92; In some embodiments, the guide sequence is SEQ ID NO: 93; In some embodiments, the guide sequence is SEQ ID NO: 94; In some embodiments, the guide sequence is SEQ ID NO: 95; In some embodiments, the guide sequence is SEQ ID NO: 96; In some embodiments, the guide sequence is SEQ ID NO: 97; In some embodiments, the guide sequence is SEQ ID NO: 98; In some embodiments, the guide sequence is SEQ ID NO: 99; In some embodiments, the guide sequence is SEQ ID NO: 100; In some embodiments, the guide sequence is SEQ ID NO: 101; In some embodiments, the guide sequence is SEQ ID NO: 102; In some embodiments, the guide sequence is SEQ ID NO: 103. In some embodiments, the guide sequence is SEQ ID NO: 104; In some embodiments, the guide sequence is SEQ ID NO: 105; In some embodiments, the guide sequence is SEQ ID NO: 106; In some embodiments, the guide sequence is SEQ ID NO: 107; In some embodiments, the guide sequence is SEQ ID NO: 108; In some embodiments, the guide sequence is SEQ ID NO: 109; In some embodiments, the guide sequence is SEQ ID NO: 110; In some embodiments, the guide sequence is SEQ ID NO: 111; In some embodiments, the guide sequence is SEQ ID NO: 112; In some embodiments, the guide sequence is SEQ ID NO: 113; In some embodiments, the guide sequence is SEQ ID NO: 114; In some embodiments, the guide sequence is SEQ ID NO: 115; In some embodiments, the guide sequence is SEQ ID NO: 116; In some embodiments, the guide sequence is SEQ ID NO: 117; In some embodiments, the guide sequence is SEQ ID NO: 118; In some embodiments, the guide sequence is SEQ ID NO: 119; In some embodiments, the guide sequence is SEQ ID NO: 120; In some embodiments, the guide sequence is SEQ ID NO: 121; In some embodiments, the guide sequence is SEQ ID NO: 122; In some embodiments, the guide sequence is SEQ ID NO: 123; In some embodiments, the guide sequence is SEQ ID NO: 124; In some embodiments, the guide sequence is SEQ ID NO: 125; In some embodiments, the guide sequence is SEQ ID NO: 126; In some embodiments, the guide sequence is SEQ ID NO: 127; In some embodiments, the guide sequence is SEQ ID NO: 128; In some embodiments, the guide sequence is SEQ ID NO: 129; In some embodiments, the guide sequence is SEQ ID NO: 130; In some embodiments, the guide sequence is SEQ ID NO: 131; In some embodiments, the guide sequence is SEQ ID NO: 132; In some embodiments, the guide sequence is SEQ ID NO: 133; In some embodiments, the guide sequence is SEQ ID NO: 134; In some embodiments, the guide sequence is SEQ ID NO: 135; In some embodiments, the guide sequence is SEQ ID NO: 136; In some embodiments, the guide sequence is SEQ ID NO: 137; In some embodiments, the guide sequence is SEQ ID NO: 138; In some embodiments, the guide sequence is SEQ ID NO: 139; In some embodiments, the guide sequence is SEQ ID NO: 140; In some embodiments, the guide sequence is SEQ ID NO: 141; In some embodiments, the guide sequence is SEQ ID NO: 142; In some embodiments, the guide sequence is SEQ ID NO: 143; In some embodiments, the guide sequence is SEQ ID NO: 144; In some embodiments, the guide sequence is SEQ ID NO: 145; In some embodiments, the guide sequence is SEQ ID NO: 146; In some embodiments, the guide sequence is SEQ ID NO: 147; In some embodiments, the guide sequence is SEQ ID NO: 148; In some embodiments, the guide sequence is SEQ ID NO: 149; In some embodiments, the guide sequence is SEQ ID NO: 150; In some embodiments, the guide sequence is SEQ ID NO: 151; In some embodiments, the guide sequence is SEQ ID NO: 152; In some embodiments, the guide sequence is SEQ ID NO: 153; In some embodiments, the guide sequence is SEQ ID NO: 154; In some embodiments, the guide sequence is SEQ ID NO: 155; In some embodiments, the guide sequence is SEQ ID NO: 156; In some embodiments, the guide sequence is SEQ ID NO: 157; In some embodiments, the guide sequence is SEQ ID NO: 158; In some embodiments, the guide sequence is SEQ ID NO: 159; In some embodiments, the guide sequence is SEQ ID NO: 201; In some embodiments, the guide sequence is SEQ ID NO: 202; In some embodiments, the guide sequence is SEQ ID NO: 203; In some embodiments, the guide sequence is SEQ ID NO: 204; In some embodiments, the guide sequence is SEQ ID NO: 205; In some embodiments, the guide sequence is SEQ ID NO: 206; In some embodiments, the guide sequence is SEQ ID NO: 207; In some embodiments, the guide sequence is SEQ ID NO: 208; In some embodiments, the guide sequence is SEQ ID NO: 209; In some embodiments, the guide sequence is SEQ ID NO: 210; In some embodiments, the guide sequence is SEQ ID NO: 211; In some embodiments, the guide sequence is SEQ ID NO: 212; In some embodiments, the guide sequence is SEQ ID NO: 213; In some embodiments, the guide sequence is SEQ ID NO: 214; In some embodiments, the guide sequence is SEQ ID NO: 215; In some embodiments, the guide sequence is SEQ ID NO: 216; In some embodiments, the guide sequence is SEQ ID NO: 217; In some embodiments, the guide sequence is SEQ ID NO: 218; In some embodiments, the guide sequence is SEQ ID NO: 219; In some embodiments, the guide sequence is SEQ ID NO: 220; In some embodiments, the guide sequence is SEQ ID NO: 221; In some embodiments, the guide sequence is SEQ ID NO: 222; In some embodiments, the guide sequence is SEQ ID NO: 223; In some embodiments, the guide sequence is SEQ ID NO: 224; In some embodiments, the guide sequence is SEQ ID NO: 225; In some embodiments, the guide sequence is SEQ ID NO: 226; In some embodiments, the guide sequence is SEQ ID NO: 227; In some embodiments, the guide sequence is SEQ ID NO: 228; In some embodiments, the guide sequence is SEQ ID NO: 229; In some embodiments, the guide sequence is SEQ ID NO: 230; In some embodiments, the guide sequence is SEQ ID NO: 231; In some embodiments, the guide sequence is SEQ ID NO: 232; In some embodiments, the guide sequence is SEQ ID NO: 233; In some embodiments, the guide sequence is SEQ ID NO: 234; In some embodiments, the guide sequence is SEQ ID NO: 235; In some embodiments, the guide sequence is SEQ ID NO: 236; In some embodiments, the guide sequence is SEQ ID NO: 237; In some embodiments, the guide sequence is SEQ ID NO: 238; In some embodiments, the guide sequence is SEQ ID NO: 239; In some embodiments, the guide sequence is SEQ ID NO: 240; In some embodiments, the guide sequence is SEQ ID NO: 241; In some embodiments, the guide sequence is SEQ ID NO: 242; In some embodiments, the guide sequence is SEQ ID NO: 243; In some embodiments, the guide sequence is SEQ ID NO: 244; In some embodiments, the guide sequence is SEQ ID NO: 245; In some embodiments, the guide sequence is SEQ ID NO: 246; In some embodiments, the guide sequence is SEQ ID NO: 247; In some embodiments, the guide sequence is SEQ ID NO: 248; In some embodiments, the guide sequence is SEQ ID NO: 249; In some embodiments, the guide sequence is SEQ ID NO: 250; In some embodiments, the guide sequence is SEQ ID NO: 251; In some embodiments, the guide sequence is SEQ ID NO: 252; In some embodiments, the guide sequence is SEQ ID NO: 253; In some embodiments, the guide sequence is SEQ ID NO: 254; In some embodiments, the guide sequence is SEQ ID NO: 255; In some embodiments, the guide sequence is SEQ ID NO: 256; In some embodiments, the guide sequence is SEQ ID NO: 257; In some embodiments, the guide sequence is SEQ ID NO: 258; In some embodiments, the guide sequence is SEQ ID NO: 259; In some embodiments, the guide sequence is SEQ ID NO: 260; In some embodiments, the guide sequence is SEQ ID NO: 261; In some embodiments, the guide sequence is SEQ ID NO: 262; In some embodiments, the guide sequence is SEQ ID NO: 263; In some embodiments, the guide sequence is SEQ ID NO: 264; In some embodiments, the guide sequence is SEQ ID NO: 265; In some embodiments, the guide sequence is SEQ ID NO: 266; In some embodiments, the guide sequence is SEQ ID NO: 267; In some embodiments, the guide sequence is SEQ ID NO: 268; In some embodiments, the guide sequence is SEQ ID NO: 269; In some embodiments, the guide sequence is SEQ ID NO: 270; In some embodiments, the guide sequence is SEQ ID NO: 271; In some embodiments, the guide sequence is SEQ ID NO: 272; In some embodiments, the guide sequence is SEQ ID NO: 273; In some embodiments, the guide sequence is SEQ ID NO: 274; In some embodiments, the guide sequence is SEQ ID NO: 275; In some embodiments, the guide sequence is SEQ ID NO: 276; In some embodiments, the guide sequence is SEQ ID NO: 277; In some embodiments, the guide sequence is SEQ ID NO: 278; In some embodiments, the guide sequence is SEQ ID NO: 279; In some embodiments, the guide sequence is SEQ ID NO: 280; In some embodiments, the guide sequence is SEQ ID NO: 281; In some embodiments, the guide sequence is SEQ ID NO: 282; In some embodiments, the guide sequence is SEQ ID NO: 283; In some embodiments, the guide sequence is SEQ ID NO: 284; In some embodiments, the guide sequence is SEQ ID NO: 285; In some embodiments, the guide sequence is SEQ ID NO: 286; In some embodiments, the guide sequence is SEQ ID NO: 287; In some embodiments, the guide sequence is SEQ ID NO: 288; In some embodiments, the guide sequence is SEQ ID NO: 289; In some embodiments, the guide sequence is SEQ ID NO: 290; In some embodiments, the guide sequence is SEQ ID NO: 291; In some embodiments, the guide sequence is SEQ ID NO: 292; In some embodiments, the guide sequence is SEQ ID NO: 924; In some embodiments, the guide sequence is SEQ ID NO: 925; In some embodiments, the guide sequence is SEQ ID NO: 926; In some embodiments, the guide sequence is SEQ ID NO: 927; In some embodiments, the guide sequence is SEQ ID NO: 928; In some embodiments, the guide sequence is SEQ ID NO: 929; In some embodiments, the guide sequence is SEQ ID NO: 930; In some embodiments, the guide sequence is SEQ ID NO: 931; In some embodiments, the guide sequence is SEQ ID NO: 932; In some embodiments, the guide sequence is SEQ ID NO: 933; In some embodiments, the guide sequence is SEQ ID NO: 934; In some embodiments, the guide sequence is SEQ ID NO: 935; In some embodiments, the guide sequence is SEQ ID NO: 936; In some embodiments, the guide sequence is SEQ ID NO: 937; In some embodiments, the guide sequence is SEQ ID NO: 938.

In some embodiments, a composition is provided comprising: a) one or more nucleic acid molecules encoding a SaCas9 or SluCas9 and b) a pair of guide sequences comprising a first guide sequence and a second guide sequence, wherein the pair is selected from any of the following pairs: SEQ ID NO: 1 and SEQ ID NO: 3; SEQ ID NO: 3 and SEQ ID NO: 4; SEQ ID NO: 3 and SEQ ID NO: 5; SEQ ID NO: 3 and SEQ ID NO: 6; SEQ ID NO: 3 and SEQ ID NO: 7; SEQ ID NO: 3 and SEQ ID NO: 8; SEQ ID NO: 3 and SEQ ID NO: 9; SEQ ID NO: 4 and SEQ ID NO: 5; SEQ ID NO: 4 and SEQ ID NO: 6; SEQ ID NO: 4 and SEQ ID NO: 7; SEQ ID NO: 4 and SEQ ID NO: 8; SEQ ID NO: 4 and SEQ ID NO: 9; SEQ ID NO: 5 and SEQ ID NO: 6; SEQ ID NO: 5 and SEQ ID NO: 7; SEQ ID NO: 5 and SEQ ID NO: 8; SEQ ID NO: 5 and SEQ ID NO: 9; SEQ ID NO: 6 and SEQ ID NO: 7; SEQ ID NO: 6 and SEQ ID NO: 8; SEQ ID NO: 6 and SEQ ID NO: 9; SEQ ID NO: 7 and SEQ ID NO: 8; SEQ ID NO: 7 and SEQ ID NO: 9; SEQ ID NO: 8 and SEQ ID NO: 9; SEQ ID NO: 11 and SEQ ID NO: 12; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 13 and SEQ ID NO: 14; SEQ ID NO: 13 and SEQ ID NO: 15; SEQ ID NO: 14 and SEQ ID NO: 15; SEQ ID NO: 16 and SEQ ID NO: 17; SEQ ID NO: 16 and SEQ ID NO: 18; SEQ ID NO: 16 and SEQ ID NO: 19; SEQ ID NO: 16 and SEQ ID NO: 20; SEQ ID NO: 16 and SEQ ID NO: 21; SEQ ID NO: 16 and SEQ ID NO: 22; SEQ ID NO: 16 and SEQ ID NO: 23; SEQ ID NO: 16 and SEQ ID NO: 24; SEQ ID NO: 16 and SEQ ID NO: 25; SEQ ID NO: 16 and SEQ ID NO: 26; SEQ ID NO: 17 and SEQ ID NO: 18; SEQ ID NO: 17 and SEQ ID NO: 19; SEQ ID NO: 17 and SEQ ID NO: 20; SEQ ID NO: 17 and SEQ ID NO: 21; SEQ ID NO: 17 and SEQ ID NO: 22; SEQ ID NO: 17 and SEQ ID NO: 23; SEQ ID NO: 17 and SEQ ID NO: 24; SEQ ID NO: 17 and SEQ ID NO: 25; SEQ ID NO: 17 and SEQ ID NO: 26; SEQ ID NO: 18 and SEQ ID NO: 19; SEQ ID NO: 18 and SEQ ID NO: 20; SEQ ID NO: 18 and SEQ ID NO: 21; SEQ ID NO: 18 and SEQ ID NO: 22; SEQ ID NO: 18 and SEQ ID NO: 23; SEQ ID NO: 18 and SEQ ID NO: 24; SEQ ID NO: 18 and SEQ ID NO: 25; SEQ ID NO: 18 and SEQ ID NO: 26; SEQ ID NO: 19 and SEQ ID NO: 20; SEQ ID NO: 19 and SEQ ID NO: 21; SEQ ID NO: 19 and SEQ ID NO: 22; SEQ ID NO: 19 and SEQ ID NO: 23; SEQ ID NO: 19 and SEQ ID NO: 24; SEQ ID NO: 19 and SEQ ID NO: 25; SEQ ID NO: 19 and SEQ ID NO: 26; SEQ ID NO: 20 and SEQ ID NO: 21; SEQ ID NO: 20 and SEQ ID NO: 22; SEQ ID NO: 20 and SEQ ID NO: 23; SEQ ID NO: 20 and SEQ ID NO: 24; SEQ ID NO: 20 and SEQ ID NO: 25; SEQ ID NO: 20 and SEQ ID NO: 26; SEQ ID NO: 21 and SEQ ID NO: 22; SEQ ID NO: 21 and SEQ ID NO: 23; SEQ ID NO: 21 and SEQ ID NO: 24; SEQ ID NO: 21 and SEQ ID NO: 25; SEQ ID NO: 21 and SEQ ID NO: 26; SEQ ID NO: 22 and SEQ ID NO: 23; SEQ ID NO: 22 and SEQ ID NO: 24; SEQ ID NO: 22 and SEQ ID NO: 25; SEQ ID NO: 22 and SEQ ID NO: 26; SEQ ID NO: 23 and SEQ ID NO: 24; SEQ ID NO: 23 and SEQ ID NO: 25; SEQ ID NO: 23 and SEQ ID NO: 26; SEQ ID NO: 24 and SEQ ID NO: 25; SEQ ID NO: 24 and SEQ ID NO: 26; SEQ ID NO: 25 and SEQ ID NO: 26; SEQ ID NO: 110 and SEQ ID NO: 120; SEQ ID NO: 110 and SEQ ID NO: 121; SEQ ID NO: 110 and SEQ ID NO: 122; SEQ ID NO: 110 and SEQ ID NO: 123; SEQ ID NO: 110 and SEQ ID NO: 124; SEQ ID NO: 110 and SEQ ID NO: 125; SEQ ID NO: 111 and SEQ ID NO: 112; SEQ ID NO: 111 and SEQ ID NO: 113; SEQ ID NO: 111 and SEQ ID NO: 114; SEQ ID NO: 111 and SEQ ID NO: 115; SEQ ID NO: 111 and SEQ ID NO: 116; SEQ ID NO: 111 and SEQ ID NO: 117; SEQ ID NO: 111 and SEQ ID NO: 118; SEQ ID NO: 111 and SEQ ID NO: 119; SEQ ID NO: 111 and SEQ ID NO: 120; SEQ ID NO: 111 and SEQ ID NO: 121; SEQ ID NO: 111 and SEQ ID NO: 122; SEQ ID NO: 111 and SEQ ID NO: 123; SEQ ID NO: 111 and SEQ ID NO: 124; SEQ ID NO: 111 and SEQ ID NO: 125; SEQ ID NO: 112 and SEQ ID NO: 113; SEQ ID NO: 112 and SEQ ID NO: 114; SEQ ID NO: 112 and SEQ ID NO: 115; SEQ ID NO: 112 and SEQ ID NO: 116; SEQ ID NO: 112 and SEQ ID NO: 117; SEQ ID NO: 112 and SEQ ID NO: 118; SEQ ID NO: 112 and SEQ ID NO: 119; SEQ ID NO: 112 and SEQ ID NO: 120; SEQ ID NO: 112 and SEQ ID NO: 121; SEQ ID NO: 112 and SEQ ID NO: 122; SEQ ID NO: 112 and SEQ ID NO: 123; SEQ ID NO: 112 and SEQ ID NO: 124; SEQ ID NO: 112 and SEQ ID NO: 125; SEQ ID NO: 113 and SEQ ID NO: 114; SEQ ID NO: 113 and SEQ ID NO: 115; SEQ ID NO: 113 and SEQ ID NO: 116; SEQ ID NO: 113 and SEQ ID NO: 117; SEQ ID NO: 113 and SEQ ID NO: 118; SEQ ID NO: 113 and SEQ ID NO: 119; SEQ ID NO: 113 and SEQ ID NO: 120; SEQ ID NO: 113 and SEQ ID NO: 121; SEQ ID NO: 113 and SEQ ID NO: 122; SEQ ID NO: 113 and SEQ ID NO: 123; SEQ ID NO: 113 and SEQ ID NO: 124; SEQ ID NO: 113 and SEQ ID NO: 125; SEQ ID NO: 114 and SEQ ID NO: 115; SEQ ID NO: 114 and SEQ ID NO: 116; SEQ ID NO: 114 and SEQ ID NO: 117; SEQ ID NO: 114 and SEQ ID NO: 118; SEQ ID NO: 114 and SEQ ID NO: 119; SEQ ID NO: 114 and SEQ ID NO: 120; SEQ ID NO: 114 and SEQ ID NO: 121; SEQ ID NO: 114 and SEQ ID NO: 122; SEQ ID NO: 114 and SEQ ID NO: 123; SEQ ID NO: 114 and SEQ ID NO: 124; SEQ ID NO: 114 and SEQ ID NO: 125; SEQ ID NO: 115 and SEQ ID NO: 116; SEQ ID NO: 115 and SEQ ID NO: 117; SEQ ID NO: 115 and SEQ ID NO: 118; SEQ ID NO: 115 and SEQ ID NO: 119; SEQ ID NO: 115 and SEQ ID NO: 120; SEQ ID NO: 115 and SEQ ID NO: 121; SEQ ID NO: 115 and SEQ ID NO: 122; SEQ ID NO: 115 and SEQ ID NO: 123; SEQ ID NO: 115 and SEQ ID NO: 124; SEQ ID NO: 115 and SEQ ID NO: 125; SEQ ID NO: 116 and SEQ ID NO: 117; SEQ ID NO: 116 and SEQ ID NO: 118; SEQ ID NO: 116 and SEQ ID NO: 119; SEQ ID NO: 116 and SEQ ID NO: 120; SEQ ID NO: 116 and SEQ ID NO: 121; SEQ ID NO: 116 and SEQ ID NO: 122; SEQ ID NO: 116 and SEQ ID NO: 123; SEQ ID NO: 116 and SEQ ID NO: 124; SEQ ID NO: 116 and SEQ ID NO: 125; SEQ ID NO: 117 and SEQ ID NO: 118; SEQ ID NO: 117 and SEQ ID NO: 119; SEQ ID NO: 117 and SEQ ID NO: 120; SEQ ID NO: 117 and SEQ ID NO: 121; SEQ ID NO: 117 and SEQ ID NO: 122; SEQ ID NO: 117 and SEQ ID NO: 123; SEQ ID NO: 117 and SEQ ID NO: 124; SEQ ID NO: 117 and SEQ ID NO: 125; SEQ ID NO: 118 and SEQ ID NO: 119; SEQ ID NO: 118 and SEQ ID NO: 120; SEQ ID NO: 118 and SEQ ID NO: 121; SEQ ID NO: 118 and SEQ ID NO: 122; SEQ ID NO: 118 and SEQ ID NO: 123; SEQ ID NO: 118 and SEQ ID NO: 124; SEQ ID NO: 118 and SEQ ID NO: 125; SEQ ID NO: 119 and SEQ ID NO: 120; SEQ ID NO: 119 and SEQ ID NO: 121; SEQ ID NO: 119 and SEQ ID NO: 122; SEQ ID NO: 119 and SEQ ID NO: 123; SEQ ID NO: 119 and SEQ ID NO: 124; SEQ ID NO: 119 and SEQ ID NO: 125; SEQ ID NO: 120 and SEQ ID NO: 121; SEQ ID NO: 120 and SEQ ID NO: 122; SEQ ID NO: 120 and SEQ ID NO: 123; SEQ ID NO: 120 and SEQ ID NO: 124; SEQ ID NO: 120 and SEQ ID NO: 125; SEQ ID NO: 121 and SEQ ID NO: 122; SEQ ID NO: 121 and SEQ ID NO: 123; SEQ ID NO: 121 and SEQ ID NO: 124; SEQ ID NO: 121 and SEQ ID NO: 125; SEQ ID NO: 122 and SEQ ID NO: 123; SEQ ID NO: 122 and SEQ ID NO: 124; SEQ ID NO: 122 and SEQ ID NO: 125; SEQ ID NO: 123 and SEQ ID NO: 124; SEQ ID NO: 123 and SEQ ID NO: 125; SEQ ID NO: 124 and SEQ ID NO: 125; SEQ ID NO: 126 and SEQ ID NO: 127; SEQ ID NO: 126 and SEQ ID NO: 128; SEQ ID NO: 126 and SEQ ID NO: 129; SEQ ID NO: 126 and SEQ ID NO: 130; SEQ ID NO: 126 and SEQ ID NO: 131; SEQ ID NO: 126 and SEQ ID NO: 132; SEQ ID NO: 126 and SEQ ID NO: 133; SEQ ID NO: 126 and SEQ ID NO: 134; SEQ ID NO: 126 and SEQ ID NO: 135; SEQ ID NO: 126 and SEQ ID NO: 136; SEQ ID NO: 126 and SEQ ID NO: 137; SEQ ID NO: 126 and SEQ ID NO: 138; SEQ ID NO: 126 and SEQ ID NO: 139; SEQ ID NO: 126 and SEQ ID NO: 140; SEQ ID NO: 126 and SEQ ID NO: 141; SEQ ID NO: 126 and SEQ ID NO: 142; SEQ ID NO: 126 and SEQ ID NO: 143; SEQ ID NO: 126 and SEQ ID NO: 144; SEQ ID NO: 126 and SEQ ID NO: 145; SEQ ID NO: 126 and SEQ ID NO: 146; SEQ ID NO: 126 and SEQ ID NO: 147; SEQ ID NO: 127 and SEQ ID NO: 128; SEQ ID NO: 127 and SEQ ID NO: 129; SEQ ID NO: 127 and SEQ ID NO: 130; SEQ ID NO: 127 and SEQ ID NO: 131; SEQ ID NO: 127 and SEQ ID NO: 132; SEQ ID NO: 127 and SEQ ID NO: 133; SEQ ID NO: 127 and SEQ ID NO: 134; SEQ ID NO: 127 and SEQ ID NO: 135; SEQ ID NO: 127 and SEQ ID NO: 136; SEQ ID NO: 127 and SEQ ID NO: 137; SEQ ID NO: 127 and SEQ ID NO: 138; SEQ ID NO: 127 and SEQ ID NO: 139; SEQ ID NO: 127 and SEQ ID NO: 140; SEQ ID NO: 127 and SEQ ID NO: 141; SEQ ID NO: 127 and SEQ ID NO: 142; SEQ ID NO: 127 and SEQ ID NO: 143; SEQ ID NO: 127 and SEQ ID NO: 144; SEQ ID NO: 127 and SEQ ID NO: 145; SEQ ID NO: 127 and SEQ ID NO: 146; SEQ ID NO: 127 and SEQ ID NO: 147; SEQ ID NO: 128 and SEQ ID NO: 129; SEQ ID NO: 128 and SEQ ID NO: 130; SEQ ID NO: 128 and SEQ ID NO: 131; SEQ ID NO: 128 and SEQ ID NO: 132; SEQ ID NO: 128 and SEQ ID NO: 133; SEQ ID NO: 128 and SEQ ID NO: 134; SEQ ID NO: 128 and SEQ ID NO: 135; SEQ ID NO: 128 and SEQ ID NO: 136; SEQ ID NO: 128 and SEQ ID NO: 137; SEQ ID NO: 128 and SEQ ID NO: 138; SEQ ID NO: 128 and SEQ ID NO: 139; SEQ ID NO: 128 and SEQ ID NO: 140; SEQ ID NO: 128 and SEQ ID NO: 141; SEQ ID NO: 128 and SEQ ID NO: 142; SEQ ID NO: 128 and SEQ ID NO: 143; SEQ ID NO: 128 and SEQ ID NO: 144; SEQ ID NO: 128 and SEQ ID NO: 145; SEQ ID NO: 128 and SEQ ID NO: 146; SEQ ID NO: 128 and SEQ ID NO: 147; SEQ ID NO: 129 and SEQ ID NO: 130; SEQ ID NO: 129 and SEQ ID NO: 131; SEQ ID NO: 129 and SEQ ID NO: 132; SEQ ID NO: 129 and SEQ ID NO: 133; SEQ ID NO: 129 and SEQ ID NO: 134; SEQ ID NO: 129 and SEQ ID NO: 135; SEQ ID NO: 129 and SEQ ID NO: 136; SEQ ID NO: 129 and SEQ ID NO: 137; SEQ ID NO: 129 and SEQ ID NO: 138; SEQ ID NO: 129 and SEQ ID NO: 139; SEQ ID NO: 129 and SEQ ID NO: 140; SEQ ID NO: 129 and SEQ ID NO: 141; SEQ ID NO: 129 and SEQ ID NO: 142; SEQ ID NO: 129 and SEQ ID NO: 143; SEQ ID NO: 129 and SEQ ID NO: 144; SEQ ID NO: 129 and SEQ ID NO: 145; SEQ ID NO: 129 and SEQ ID NO: 146; SEQ ID NO: 129 and SEQ ID NO: 147; SEQ ID NO: 130 and SEQ ID NO: 131; SEQ ID NO: 130 and SEQ ID NO: 132; SEQ ID NO: 130 and SEQ ID NO: 133; SEQ ID NO: 130 and SEQ ID NO: 134; SEQ ID NO: 130 and SEQ ID NO: 135; SEQ ID NO: 130 and SEQ ID NO: 136; SEQ ID NO: 130 and SEQ ID NO: 137; SEQ ID NO: 130 and SEQ ID NO: 138; SEQ ID NO: 130 and SEQ ID NO: 139; SEQ ID NO: 130 and SEQ ID NO: 140; SEQ ID NO: 130 and SEQ ID NO: 141; SEQ ID NO: 130 and SEQ ID NO: 142; SEQ ID NO: 130 and SEQ ID NO: 143; SEQ ID NO: 130 and SEQ ID NO: 144; SEQ ID NO: 130 and SEQ ID NO: 145; SEQ ID NO: 130 and SEQ ID NO: 146; SEQ ID NO: 130 and SEQ ID NO: 147; SEQ ID NO: 131 and SEQ ID NO: 132; SEQ ID NO: 131 and SEQ ID NO: 133; SEQ ID NO: 131 and SEQ ID NO: 134; SEQ ID NO: 131 and SEQ ID NO: 135; SEQ ID NO: 131 and SEQ ID NO: 136; SEQ ID NO: 131 and SEQ ID NO: 137; SEQ ID NO: 131 and SEQ ID NO: 138; SEQ ID NO: 131 and SEQ ID NO: 139; SEQ ID NO: 131 and SEQ ID NO: 140; SEQ ID NO: 131 and SEQ ID NO: 141; SEQ ID NO: 131 and SEQ ID NO: 142; SEQ ID NO: 131 and SEQ ID NO: 143; SEQ ID NO: 131 and SEQ ID NO: 144; SEQ ID NO: 131 and SEQ ID NO: 145; SEQ ID NO: 131 and SEQ ID NO: 146; SEQ ID NO: 131 and SEQ ID NO: 147; SEQ ID NO: 132 and SEQ ID NO: 133; SEQ ID NO: 132 and SEQ ID NO: 134; SEQ ID NO: 132 and SEQ ID NO: 135; SEQ ID NO: 132 and SEQ ID NO: 136; SEQ ID NO: 132 and SEQ ID NO: 137; SEQ ID NO: 132 and SEQ ID NO: 138; SEQ ID NO: 132 and SEQ ID NO: 139; SEQ ID NO: 132 and SEQ ID NO: 140; SEQ ID NO: 132 and SEQ ID NO: 141; SEQ ID NO: 132 and SEQ ID NO: 142; SEQ ID NO: 132 and SEQ ID NO: 143; SEQ ID NO: 132 and SEQ ID NO: 144; SEQ ID NO: 132 and SEQ ID NO: 145; SEQ ID NO: 132 and SEQ ID NO: 146; SEQ ID NO: 132 and SEQ ID NO: 147; SEQ ID NO: 133 and SEQ ID NO: 134; SEQ ID NO: 133 and SEQ ID NO: 135; SEQ ID NO: 133 and SEQ ID NO: 136; SEQ ID NO: 133 and SEQ ID NO: 137; SEQ ID NO: 133 and SEQ ID NO: 138; SEQ ID NO: 133 and SEQ ID NO: 139; SEQ ID NO: 133 and SEQ ID NO: 140; SEQ ID NO: 133 and SEQ ID NO: 141; SEQ ID NO: 133 and SEQ ID NO: 142; SEQ ID NO: 133 and SEQ ID NO: 143; SEQ ID NO: 133 and SEQ ID NO: 144; SEQ ID NO: 133 and SEQ ID NO: 145; SEQ ID NO: 133 and SEQ ID NO: 146; SEQ ID NO: 133 and SEQ ID NO: 147; SEQ ID NO: 134 and SEQ ID NO: 135; SEQ ID NO: 134 and SEQ ID NO: 136; SEQ ID NO: 134 and SEQ ID NO: 137; SEQ ID NO: 134 and SEQ ID NO: 138; SEQ ID NO: 134 and SEQ ID NO: 139; SEQ ID NO: 134 and SEQ ID NO: 140; SEQ ID NO: 134 and SEQ ID NO: 141; SEQ ID NO: 134 and SEQ ID NO: 142; SEQ ID NO: 134 and SEQ ID NO: 143; SEQ ID NO: 134 and SEQ ID NO: 144; SEQ ID NO: 134 and SEQ ID NO: 145; SEQ ID NO: 134 and SEQ ID NO: 146; SEQ ID NO: 134 and SEQ ID NO: 147; SEQ ID NO: 135 and SEQ ID NO: 136; SEQ ID NO: 135 and SEQ ID NO: 137; SEQ ID NO: 135 and SEQ ID NO: 138; SEQ ID NO: 135 and SEQ ID NO: 139; SEQ ID NO: 135 and SEQ ID NO: 140; SEQ ID NO: 135 and SEQ ID NO: 141; SEQ ID NO: 135 and SEQ ID NO: 142; SEQ ID NO: 135 and SEQ ID NO: 143; SEQ ID NO: 135 and SEQ ID NO: 144; SEQ ID NO: 135 and SEQ ID NO: 145; SEQ ID NO: 135 and SEQ ID NO: 146; SEQ ID NO: 135 and SEQ ID NO: 147; SEQ ID NO: 136 and SEQ ID NO: 137; SEQ ID NO: 136 and SEQ ID NO: 138; SEQ ID NO: 136 and SEQ ID NO: 139; SEQ ID NO: 136 and SEQ ID NO: 140; SEQ ID NO: 136 and SEQ ID NO: 141; SEQ ID NO: 136 and SEQ ID NO: 142; SEQ ID NO: 136 and SEQ ID NO: 143; SEQ ID NO: 136 and SEQ ID NO: 144; SEQ ID NO: 136 and SEQ ID NO: 145; SEQ ID NO: 136 and SEQ ID NO: 146; SEQ ID NO: 136 and SEQ ID NO: 147; SEQ ID NO: 137 and SEQ ID NO: 138; SEQ ID NO: 137 and SEQ ID NO: 139; SEQ ID NO: 137 and SEQ ID NO: 140; SEQ ID NO: 137 and SEQ ID NO: 141; SEQ ID NO: 137 and SEQ ID NO: 142; SEQ ID NO: 137 and SEQ ID NO: 143; SEQ ID NO: 137 and SEQ ID NO: 144; SEQ ID NO: 137 and SEQ ID NO: 145; SEQ ID NO: 137 and SEQ ID NO: 146; SEQ ID NO: 137 and SEQ ID NO: 147; SEQ ID NO: 138 and SEQ ID NO: 139; SEQ ID NO: 138 and SEQ ID NO: 140; SEQ ID NO: 138 and SEQ ID NO: 141; SEQ ID NO: 138 and SEQ ID NO: 142; SEQ ID NO: 138 and SEQ ID NO: 143; SEQ ID NO: 138 and SEQ ID NO: 144; SEQ ID NO: 138 and SEQ ID NO: 145; SEQ ID NO: 138 and SEQ ID NO: 146; SEQ ID NO: 138 and SEQ ID NO: 147; SEQ ID NO: 139 and SEQ ID NO: 140; SEQ ID NO: 139 and SEQ ID NO: 141; SEQ ID NO: 139 and SEQ ID NO: 142; SEQ ID NO: 139 and SEQ ID NO: 143; SEQ ID NO: 139 and SEQ ID NO: 144; SEQ ID NO: 139 and SEQ ID NO: 145; SEQ ID NO: 139 and SEQ ID NO: 146; SEQ ID NO: 139 and SEQ ID NO: 147; SEQ ID NO: 140 and SEQ ID NO: 141; SEQ ID NO: 140 and SEQ ID NO: 142; SEQ ID NO: 140 and SEQ ID NO: 143; SEQ ID NO: 140 and SEQ ID NO: 144; SEQ ID NO: 140 and SEQ ID NO: 145; SEQ ID NO: 140 and SEQ ID NO: 146; SEQ ID NO: 140 and SEQ ID NO: 147; SEQ ID NO: 141 and SEQ ID NO: 142; SEQ ID NO: 141 and SEQ ID NO: 143; SEQ ID NO: 141 and SEQ ID NO: 144; SEQ ID NO: 141 and SEQ ID NO: 145; SEQ ID NO: 141 and SEQ ID NO: 146; SEQ ID NO: 141 and SEQ ID NO: 147; SEQ ID NO: 142 and SEQ ID NO: 143; SEQ ID NO: 142 and SEQ ID NO: 144; SEQ ID NO: 142 and SEQ ID NO: 145; SEQ ID NO: 142 and SEQ ID NO: 146; SEQ ID NO: 142 and SEQ ID NO: 147; SEQ ID NO: 143 and SEQ ID NO: 144; SEQ ID NO: 143 and SEQ ID NO: 145; SEQ ID NO: 143 and SEQ ID NO: 146; SEQ ID NO: 143 and SEQ ID NO: 147; SEQ ID NO: 144 and SEQ ID NO: 145; SEQ ID NO: 144 and SEQ ID NO: 146; SEQ ID NO: 144 and SEQ ID NO: 147; SEQ ID NO: 145 and SEQ ID NO: 146; SEQ ID NO: 145 and SEQ ID NO: 147; SEQ ID NO: 146 and SEQ ID NO: 147; SEQ ID NO: 148 and SEQ ID NO: 149; SEQ ID NO: 148 and SEQ ID NO: 150; SEQ ID NO: 148 and SEQ ID NO: 151; SEQ ID NO: 148 and SEQ ID NO: 152; SEQ ID NO: 148 and SEQ ID NO: 153; SEQ ID NO: 148 and SEQ ID NO: 154; SEQ ID NO: 148 and SEQ ID NO: 155; SEQ ID NO: 148 and SEQ ID NO: 156; SEQ ID NO: 148 and SEQ ID NO: 157; SEQ ID NO: 148 and SEQ ID NO: 158; SEQ ID NO: 148 and SEQ ID NO: 159; SEQ ID NO: 149 and SEQ ID NO: 150; SEQ ID NO: 149 and SEQ ID NO: 151; SEQ ID NO: 149 and SEQ ID NO: 152; SEQ ID NO: 149 and SEQ ID NO: 153; SEQ ID NO: 149 and SEQ ID NO: 154; SEQ ID NO: 149 and SEQ ID NO: 155; SEQ ID NO: 149 and SEQ ID NO: 156; SEQ ID NO: 149 and SEQ ID NO: 157; SEQ ID NO: 149 and SEQ ID NO: 158; SEQ ID NO: 149 and SEQ ID NO: 159; SEQ ID NO: 150 and SEQ ID NO: 151; SEQ ID NO: 150 and SEQ ID NO: 152; SEQ ID NO: 150 and SEQ ID NO: 153; SEQ ID NO: 150 and SEQ ID NO: 154; SEQ ID NO: 150 and SEQ ID NO: 155; SEQ ID NO: 150 and SEQ ID NO: 156; SEQ ID NO: 150 and SEQ ID NO: 157; SEQ ID NO: 150 and SEQ ID NO: 158; SEQ ID NO: 150 and SEQ ID NO: 159; SEQ ID NO: 151 and SEQ ID NO: 152; SEQ ID NO: 151 and SEQ ID NO: 153; SEQ ID NO: 151 and SEQ ID NO: 154; SEQ ID NO: 151 and SEQ ID NO: 155; SEQ ID NO: 151 and SEQ ID NO: 156; SEQ ID NO: 151 and SEQ ID NO: 157; SEQ ID NO: 151 and SEQ ID NO: 158; SEQ ID NO: 151 and SEQ ID NO: 159; SEQ ID NO: 152 and SEQ ID NO: 153; SEQ ID NO: 152 and SEQ ID NO: 154; SEQ ID NO: 152 and SEQ ID NO: 155; SEQ ID NO: 152 and SEQ ID NO: 156; SEQ ID NO: 152 and SEQ ID NO: 157; SEQ ID NO: 152 and SEQ ID NO: 158; SEQ ID NO: 152 and SEQ ID NO: 159; SEQ ID NO: 153 and SEQ ID NO: 154; SEQ ID NO: 153 and SEQ ID NO: 155; SEQ ID NO: 153 and SEQ ID NO: 156; SEQ ID NO: 153 and SEQ ID NO: 157; SEQ ID NO: 153 and SEQ ID NO: 158; SEQ ID NO: 153 and SEQ ID NO: 159; SEQ ID NO: 154 and SEQ ID NO: 155; SEQ ID NO: 154 and SEQ ID NO: 156; SEQ ID NO: 154 and SEQ ID NO: 157; SEQ ID NO: 154 and SEQ ID NO: 158; SEQ ID NO: 154 and SEQ ID NO: 159; SEQ ID NO: 155 and SEQ ID NO: 156; SEQ ID NO: 155 and SEQ ID NO: 157; SEQ ID NO: 155 and SEQ ID NO: 158; SEQ ID NO: 155 and SEQ ID NO: 159; SEQ ID NO: 156 and SEQ ID NO: 157; SEQ ID NO: 156 and SEQ ID NO: 158; SEQ ID NO: 156 and SEQ ID NO: 159; SEQ ID NO: 157 and SEQ ID NO: 158; SEQ ID NO: 157 and SEQ ID NO: 159; SEQ ID NO: 158 and SEQ ID NO: 159; SEQ ID NO: 27 and SEQ ID NO: 28; SEQ ID NO: 27 and SEQ ID NO: 29; SEQ ID NO: 27 and SEQ ID NO: 30; SEQ ID NO: 27 and SEQ ID NO: 31; SEQ ID NO: 27 and SEQ ID NO: 32; SEQ ID NO: 27 and SEQ ID NO: 33; SEQ ID NO: 27 and SEQ ID NO: 34; SEQ ID NO: 27 and SEQ ID NO: 35; SEQ ID NO: 27 and SEQ ID NO: 36; SEQ ID NO: 27 and SEQ ID NO: 37; SEQ ID NO: 27 and SEQ ID NO: 38; SEQ ID NO: 27 and SEQ ID NO: 39; SEQ ID NO: 27 and SEQ ID NO: 40; SEQ ID NO: 27 and SEQ ID NO: 41; SEQ ID NO: 27 and SEQ ID NO: 42; SEQ ID NO: 27 and SEQ ID NO: 43; SEQ ID NO: 27 and SEQ ID NO: 44; SEQ ID NO: 27 and SEQ ID NO: 45; SEQ ID NO: 27 and SEQ ID NO: 46; SEQ ID NO: 27 and SEQ ID NO: 47; SEQ ID NO: 27 and SEQ ID NO: 48; SEQ ID NO: 27 and SEQ ID NO: 53; SEQ ID NO: 27 and SEQ ID NO: 54; SEQ ID NO: 27 and SEQ ID NO: 55; SEQ ID NO: 27 and SEQ ID NO: 56; SEQ ID NO: 27 and SEQ ID NO: 57; SEQ ID NO: 27 and SEQ ID NO: 58; SEQ ID NO: 27 and SEQ ID NO: 59; SEQ ID NO: 27 and SEQ ID NO: 60; SEQ ID NO: 27 and SEQ ID NO: 61; SEQ ID NO: 27 and SEQ ID NO: 62; SEQ ID NO: 27 and SEQ ID NO: 63; SEQ ID NO: 27 and SEQ ID NO: 64; SEQ ID NO: 27 and SEQ ID NO: 65; SEQ ID NO: 27 and SEQ ID NO: 66; SEQ ID NO: 27 and SEQ ID NO: 67; SEQ ID NO: 27 and SEQ ID NO: 68; SEQ ID NO: 27 and SEQ ID NO: 69; SEQ ID NO: 28 and SEQ ID NO: 29; SEQ ID NO: 28 and SEQ ID NO: 30; SEQ ID NO: 28 and SEQ ID NO: 31; SEQ ID NO: 28 and SEQ ID NO: 32; SEQ ID NO: 28 and SEQ ID NO: 33; SEQ ID NO: 28 and SEQ ID NO: 34; SEQ ID NO: 28 and SEQ ID NO: 35; SEQ ID NO: 28 and SEQ ID NO: 36; SEQ ID NO: 28 and SEQ ID NO: 37; SEQ ID NO: 28 and SEQ ID NO: 38; SEQ ID NO: 28 and SEQ ID NO: 39; SEQ ID NO: 28 and SEQ ID NO: 40; SEQ ID NO: 28 and SEQ ID NO: 41; SEQ ID NO: 28 and SEQ ID NO: 42; SEQ ID NO: 28 and SEQ ID NO: 43; SEQ ID NO: 28 and SEQ ID NO: 44; SEQ ID NO: 28 and SEQ ID NO: 45; SEQ ID NO: 28 and SEQ ID NO: 46; SEQ ID NO: 28 and SEQ ID NO: 47; SEQ ID NO: 28 and SEQ ID NO: 48; SEQ ID NO: 28 and SEQ ID NO: 53; SEQ ID NO: 28 and SEQ ID NO: 54; SEQ ID NO: 28 and SEQ ID NO: 55; SEQ ID NO: 28 and SEQ ID NO: 56; SEQ ID NO: 28 and SEQ ID NO: 57; SEQ ID NO: 28 and SEQ ID NO: 58; SEQ ID NO: 28 and SEQ ID NO: 59; SEQ ID NO: 28 and SEQ ID NO: 60; SEQ ID NO: 28 and SEQ ID NO: 61; SEQ ID NO: 28 and SEQ ID NO: 62; SEQ ID NO: 28 and SEQ ID NO: 63; SEQ ID NO: 28 and SEQ ID NO: 64; SEQ ID NO: 28 and SEQ ID NO: 65; SEQ ID NO: 28 and SEQ ID NO: 66; SEQ ID NO: 28 and SEQ ID NO: 67; SEQ ID NO: 28 and SEQ ID NO: 68; SEQ ID NO: 28 and SEQ ID NO: 69; SEQ ID NO: 29 and SEQ ID NO: 30; SEQ ID NO: 29 and SEQ ID NO: 31; SEQ ID NO: 29 and SEQ ID NO: 32; SEQ ID NO: 29 and SEQ ID NO: 33; SEQ ID NO: 29 and SEQ ID NO: 34; SEQ ID NO: 29 and SEQ ID NO: 35; SEQ ID NO: 29 and SEQ ID NO: 36; SEQ ID NO: 29 and SEQ ID NO: 37; SEQ ID NO: 29 and SEQ ID NO: 38; SEQ ID NO: 29 and SEQ ID NO: 39; SEQ ID NO: 29 and SEQ ID NO: 40; SEQ ID NO: 29 and SEQ ID NO: 41; SEQ ID NO: 29 and SEQ ID NO: 42; SEQ ID NO: 29 and SEQ ID NO: 43; SEQ ID NO: 29 and SEQ ID NO: 44; SEQ ID NO: 29 and SEQ ID NO: 45; SEQ ID NO: 29 and SEQ ID NO: 46; SEQ ID NO: 29 and SEQ ID NO: 47; SEQ ID NO: 29 and SEQ ID NO: 48; SEQ ID NO: 29 and SEQ ID NO: 53; SEQ ID NO: 29 and SEQ ID NO: 54; SEQ ID NO: 29 and SEQ ID NO: 55; SEQ ID NO: 29 and SEQ ID NO: 56; SEQ ID NO: 29 and SEQ ID NO: 57; SEQ ID NO: 29 and SEQ ID NO: 58; SEQ ID NO: 29 and SEQ ID NO: 59; SEQ ID NO: 29 and SEQ ID NO: 60; SEQ ID NO: 29 and SEQ ID NO: 61; SEQ ID NO: 29 and SEQ ID NO: 62; SEQ ID NO: 29 and SEQ ID NO: 63; SEQ ID NO: 29 and SEQ ID NO: 64; SEQ ID NO: 29 and SEQ ID NO: 65; SEQ ID NO: 29 and SEQ ID NO: 66; SEQ ID NO: 29 and SEQ ID NO: 67; SEQ ID NO: 29 and SEQ ID NO: 68; SEQ ID NO: 29 and SEQ ID NO: 69; SEQ ID NO: 30 and SEQ ID NO: 31; SEQ ID NO: 30 and SEQ ID NO: 32; SEQ ID NO: 30 and SEQ ID NO: 33; SEQ ID NO: 30 and SEQ ID NO: 34; SEQ ID NO: 30 and SEQ ID NO: 35; SEQ ID NO: 30 and SEQ ID NO: 36; SEQ ID NO: 30 and SEQ ID NO: 37; SEQ ID NO: 30 and SEQ ID NO: 38; SEQ ID NO: 30 and SEQ ID NO: 39; SEQ ID NO: 30 and SEQ ID NO: 40; SEQ ID NO: 30 and SEQ ID NO: 41; SEQ ID NO: 30 and SEQ ID NO: 42; SEQ ID NO: 30 and SEQ ID NO: 43; SEQ ID NO: 30 and SEQ ID NO: 44; SEQ ID NO: 30 and SEQ ID NO: 45; SEQ ID NO: 30 and SEQ ID NO: 46; SEQ ID NO: 30 and SEQ ID NO: 47; SEQ ID NO: 30 and SEQ ID NO: 48; SEQ ID NO: 30 and SEQ ID NO: 53; SEQ ID NO: 30 and SEQ ID NO: 54; SEQ ID NO: 30 and SEQ ID NO: 55; SEQ ID NO: 30 and SEQ ID NO: 56; SEQ ID NO: 30 and SEQ ID NO: 57; SEQ ID NO: 30 and SEQ ID NO: 58; SEQ ID NO: 30 and SEQ ID NO: 59; SEQ ID NO: 30 and SEQ ID NO: 60; SEQ ID NO: 30 and SEQ ID NO: 61; SEQ ID NO: 30 and SEQ ID NO: 62; SEQ ID NO: 30 and SEQ ID NO: 63; SEQ ID NO: 30 and SEQ ID NO: 64; SEQ ID NO: 30 and SEQ ID NO: 65; SEQ ID NO: 30 and SEQ ID NO: 66; SEQ ID NO: 30 and SEQ ID NO: 67; SEQ ID NO: 30 and SEQ ID NO: 68; SEQ ID NO: 30 and SEQ ID NO: 69; SEQ ID NO: 31 and SEQ ID NO: 32; SEQ ID NO: 31 and SEQ ID NO: 33; SEQ ID NO: 31 and SEQ ID NO: 34; SEQ ID NO: 31 and SEQ ID NO: 35; SEQ ID NO: 31 and SEQ ID NO: 36; SEQ ID NO: 31 and SEQ ID NO: 37; SEQ ID NO: 31 and SEQ ID NO: 38; SEQ ID NO: 31 and SEQ ID NO: 39; SEQ ID NO: 31 and SEQ ID NO: 40; SEQ ID NO: 31 and SEQ ID NO: 41; SEQ ID NO: 31 and SEQ ID NO: 42; SEQ ID NO: 31 and SEQ ID NO: 43; SEQ ID NO: 31 and SEQ ID NO: 44; SEQ ID NO: 31 and SEQ ID NO: 45; SEQ ID NO: 31 and SEQ ID NO: 46; SEQ ID NO: 31 and SEQ ID NO: 47; SEQ ID NO: 31 and SEQ ID NO: 48; SEQ ID NO: 31 and SEQ ID NO: 53; SEQ ID NO: 31 and SEQ ID NO: 54; SEQ ID NO: 31 and SEQ ID NO: 55; SEQ ID NO: 31 and SEQ ID NO: 56; SEQ ID NO: 31 and SEQ ID NO: 57; SEQ ID NO: 31 and SEQ ID NO: 58; SEQ ID NO: 31 and SEQ ID NO: 59; SEQ ID NO: 31 and SEQ ID NO: 60; SEQ ID NO: 31 and SEQ ID NO: 61; SEQ ID NO: 31 and SEQ ID NO: 62; SEQ ID NO: 31 and SEQ ID NO: 63; SEQ ID NO: 31 and SEQ ID NO: 64; SEQ ID NO: 31 and SEQ ID NO: 65; SEQ ID NO: 31 and SEQ ID NO: 66; SEQ ID NO: 31 and SEQ ID NO: 67; SEQ ID NO: 31 and SEQ ID NO: 68; SEQ ID NO: 31 and SEQ ID NO: 69; SEQ ID NO: 32 and SEQ ID NO: 33; SEQ ID NO: 32 and SEQ ID NO: 34; SEQ ID NO: 32 and SEQ ID NO: 35; SEQ ID NO: 32 and SEQ ID NO: 36; SEQ ID NO: 32 and SEQ ID NO: 37; SEQ ID NO: 32 and SEQ ID NO: 38; SEQ ID NO: 32 and SEQ ID NO: 39; SEQ ID NO: 32 and SEQ ID NO: 40; SEQ ID NO: 32 and SEQ ID NO: 41; SEQ ID NO: 32 and SEQ ID NO: 42; SEQ ID NO: 32 and SEQ ID NO: 43; SEQ ID NO: 32 and SEQ ID NO: 44; SEQ ID NO: 32 and SEQ ID NO: 45; SEQ ID NO: 32 and SEQ ID NO: 46; SEQ ID NO: 32 and SEQ ID NO: 47; SEQ ID NO: 32 and SEQ ID NO: 48; SEQ ID NO: 32 and SEQ ID NO: 53; SEQ ID NO: 32 and SEQ ID NO: 54; SEQ ID NO: 32 and SEQ ID NO: 55; SEQ ID NO: 32 and SEQ ID NO: 56; SEQ ID NO: 32 and SEQ ID NO: 57; SEQ ID NO: 32 and SEQ ID NO: 58; SEQ ID NO: 32 and SEQ ID NO: 59; SEQ ID NO: 32 and SEQ ID NO: 60; SEQ ID NO: 32 and SEQ ID NO: 61; SEQ ID NO: 32 and SEQ ID NO: 62; SEQ ID NO: 32 and SEQ ID NO: 63; SEQ ID NO: 32 and SEQ ID NO: 64; SEQ ID NO: 32 and SEQ ID NO: 65; SEQ ID NO: 32 and SEQ ID NO: 66; SEQ ID NO: 32 and SEQ ID NO: 67; SEQ ID NO: 32 and SEQ ID NO: 68; SEQ ID NO: 32 and SEQ ID NO: 69; SEQ ID NO: 33 and SEQ ID NO: 34; SEQ ID NO: 33 and SEQ ID NO: 35; SEQ ID NO: 33 and SEQ ID NO: 36; SEQ ID NO: 33 and SEQ ID NO: 37; SEQ ID NO: 33 and SEQ ID NO: 38; SEQ ID NO: 33 and SEQ ID NO: 39; SEQ ID NO: 33 and SEQ ID NO: 40; SEQ ID NO: 33 and SEQ ID NO: 41; SEQ ID NO: 33 and SEQ ID NO: 42; SEQ ID NO: 33 and SEQ ID NO: 43; SEQ ID NO: 33 and SEQ ID NO: 44; SEQ ID NO: 33 and SEQ ID NO: 45; SEQ ID NO: 33 and SEQ ID NO: 46; SEQ ID NO: 33 and SEQ ID NO: 47; SEQ ID NO: 33 and SEQ ID NO: 48; SEQ ID NO: 33 and SEQ ID NO: 53; SEQ ID NO: 33 and SEQ ID NO: 54; SEQ ID NO: 33 and SEQ ID NO: 55; SEQ ID NO: 33 and SEQ ID NO: 56; SEQ ID NO: 33 and SEQ ID NO: 57; SEQ ID NO: 33 and SEQ ID NO: 58; SEQ ID NO: 33 and SEQ ID NO: 59; SEQ ID NO: 33 and SEQ ID NO: 60; SEQ ID NO: 33 and SEQ ID NO: 61; SEQ ID NO: 33 and SEQ ID NO: 62; SEQ ID NO: 33 and SEQ ID NO: 63; SEQ ID NO: 33 and SEQ ID NO: 64; SEQ ID NO: 33 and SEQ ID NO: 65; SEQ ID NO: 33 and SEQ ID NO: 66; SEQ ID NO: 33 and SEQ ID NO: 67; SEQ ID NO: 33 and SEQ ID NO: 68; SEQ ID NO: 33 and SEQ ID NO: 69; SEQ ID NO: 34 and SEQ ID NO: 35; SEQ ID NO: 34 and SEQ ID NO: 36; SEQ ID NO: 34 and SEQ ID NO: 37; SEQ ID NO: 34 and SEQ ID NO: 38; SEQ ID NO: 34 and SEQ ID NO: 39; SEQ ID NO: 34 and SEQ ID NO: 40; SEQ ID NO: 34 and SEQ ID NO: 41; SEQ ID NO: 34 and SEQ ID NO: 42; SEQ ID NO: 34 and SEQ ID NO: 43; SEQ ID NO: 34 and SEQ ID NO: 44; SEQ ID NO: 34 and SEQ ID NO: 45; SEQ ID NO: 34 and SEQ ID NO: 46; SEQ ID NO: 34 and SEQ ID NO: 47; SEQ ID NO: 34 and SEQ ID NO: 48; SEQ ID NO: 34 and SEQ ID NO: 53; SEQ ID NO: 34 and SEQ ID NO: 54; SEQ ID NO: 34 and SEQ ID NO: 55; SEQ ID NO: 34 and SEQ ID NO: 56; SEQ ID NO: 34 and SEQ ID NO: 57; SEQ ID NO: 34 and SEQ ID NO: 58; SEQ ID NO: 34 and SEQ ID NO: 59; SEQ ID NO: 34 and SEQ ID NO: 60; SEQ ID NO: 34 and SEQ ID NO: 61; SEQ ID NO: 34 and SEQ ID NO: 62; SEQ ID NO: 34 and SEQ ID NO: 63; SEQ ID NO: 34 and SEQ ID NO: 64; SEQ ID NO: 34 and SEQ ID NO: 65; SEQ ID NO: 34 and SEQ ID NO: 66; SEQ ID NO: 34 and SEQ ID NO: 67; SEQ ID NO: 34 and SEQ ID NO: 68; SEQ ID NO: 34 and SEQ ID NO: 69; SEQ ID NO: 35 and SEQ ID NO: 36; SEQ ID NO: 35 and SEQ ID NO: 37; SEQ ID NO: 35 and SEQ ID NO: 38; SEQ ID NO: 35 and SEQ ID NO: 39; SEQ ID NO: 35 and SEQ ID NO: 40; SEQ ID NO: 35 and SEQ ID NO: 41; SEQ ID NO: 35 and SEQ ID NO: 42; SEQ ID NO: 35 and SEQ ID NO: 43; SEQ ID NO: 35 and SEQ ID NO: 44; SEQ ID NO: 35 and SEQ ID NO: 45; SEQ ID NO: 35 and SEQ ID NO: 46; SEQ ID NO: 35 and SEQ ID NO: 47; SEQ ID NO: 35 and SEQ ID NO: 48; SEQ ID NO: 35 and SEQ ID NO: 53; SEQ ID NO: 35 and SEQ ID NO: 54; SEQ ID NO: 35 and SEQ ID NO: 55; SEQ ID NO: 35 and SEQ ID NO: 56; SEQ ID NO: 35 and SEQ ID NO: 57; SEQ ID NO: 35 and SEQ ID NO: 58; SEQ ID NO: 35 and SEQ ID NO: 59; SEQ ID NO: 35 and SEQ ID NO: 60; SEQ ID NO: 35 and SEQ ID NO: 61; SEQ ID NO: 35 and SEQ ID NO: 62; SEQ ID NO: 35 and SEQ ID NO: 63; SEQ ID NO: 35 and SEQ ID NO: 64; SEQ ID NO: 35 and SEQ ID NO: 65; SEQ ID NO: 35 and SEQ ID NO: 66; SEQ ID NO: 35 and SEQ ID NO: 67; SEQ ID NO: 35 and SEQ ID NO: 68; SEQ ID NO: 35 and SEQ ID NO: 69; SEQ ID NO: 36 and SEQ ID NO: 37; SEQ ID NO: 36 and SEQ ID NO: 38; SEQ ID NO: 36 and SEQ ID NO: 39; SEQ ID NO: 36 and SEQ ID NO: 40; SEQ ID NO: 36 and SEQ ID NO: 41; SEQ ID NO: 36 and SEQ ID NO: 42; SEQ ID NO: 36 and SEQ ID NO: 43; SEQ ID NO: 36 and SEQ ID NO: 44; SEQ ID NO: 36 and SEQ ID NO: 45; SEQ ID NO: 36 and SEQ ID NO: 46; SEQ ID NO: 36 and SEQ ID NO: 47; SEQ ID NO: 36 and SEQ ID NO: 48; SEQ ID NO: 36 and SEQ ID NO: 53; SEQ ID NO: 36 and SEQ ID NO: 54; SEQ ID NO: 36 and SEQ ID NO: 55; SEQ ID NO: 36 and SEQ ID NO: 56; SEQ ID NO: 36 and SEQ ID NO: 57; SEQ ID NO: 36 and SEQ ID NO: 58; SEQ ID NO: 36 and SEQ ID NO: 59; SEQ ID NO: 36 and SEQ ID NO: 60; SEQ ID NO: 36 and SEQ ID NO: 61; SEQ ID NO: 36 and SEQ ID NO: 62; SEQ ID NO: 36 and SEQ ID NO: 63; SEQ ID NO: 36 and SEQ ID NO: 64; SEQ ID NO: 36 and SEQ ID NO: 65; SEQ ID NO: 36 and SEQ ID NO: 66; SEQ ID NO: 36 and SEQ ID NO: 67; SEQ ID NO: 36 and SEQ ID NO: 68; SEQ ID NO: 36 and SEQ ID NO: 69; SEQ ID NO: 37 and SEQ ID NO: 38; SEQ ID NO: 37 and SEQ ID NO: 39; SEQ ID NO: 37 and SEQ ID NO: 40; SEQ ID NO: 37 and SEQ ID NO: 41; SEQ ID NO: 37 and SEQ ID NO: 42; SEQ ID NO: 37 and SEQ ID NO: 43; SEQ ID NO: 37 and SEQ ID NO: 44; SEQ ID NO: 37 and SEQ ID NO: 45; SEQ ID NO: 37 and SEQ ID NO: 46; SEQ ID NO: 37 and SEQ ID NO: 47; SEQ ID NO: 37 and SEQ ID NO: 48; SEQ ID NO: 37 and SEQ ID NO: 53; SEQ ID NO: 37 and SEQ ID NO: 54; SEQ ID NO: 37 and SEQ ID NO: 55; SEQ ID NO: 37 and SEQ ID NO: 56; SEQ ID NO: 37 and SEQ ID NO: 57; SEQ ID NO: 37 and SEQ ID NO: 58; SEQ ID NO: 37 and SEQ ID NO: 59; SEQ ID NO: 37 and SEQ ID NO: 60; SEQ ID NO: 37 and SEQ ID NO: 61; SEQ ID NO: 37 and SEQ ID NO: 62; SEQ ID NO: 37 and SEQ ID NO: 63; SEQ ID NO: 37 and SEQ ID NO: 64; SEQ ID NO: 37 and SEQ ID NO: 65; SEQ ID NO: 37 and SEQ ID NO: 66; SEQ ID NO: 37 and SEQ ID NO: 67; SEQ ID NO: 37 and SEQ ID NO: 68; SEQ ID NO: 37 and SEQ ID NO: 69; SEQ ID NO: 38 and SEQ ID NO: 39; SEQ ID NO: 38 and SEQ ID NO: 40; SEQ ID NO: 38 and SEQ ID NO: 41; SEQ ID NO: 38 and SEQ ID NO: 42; SEQ ID NO: 38 and SEQ ID NO: 43; SEQ ID NO: 38 and SEQ ID NO: 44; SEQ ID NO: 38 and SEQ ID NO: 45; SEQ ID NO: 38 and SEQ ID NO: 46; SEQ ID NO: 38 and SEQ ID NO: 47; SEQ ID NO: 38 and SEQ ID NO: 48; SEQ ID NO: 38 and SEQ ID NO: 53; SEQ ID NO: 38 and SEQ ID NO: 54; SEQ ID NO: 38 and SEQ ID NO: 55; SEQ ID NO: 38 and SEQ ID NO: 56; SEQ ID NO: 38 and SEQ ID NO: 57; SEQ ID NO: 38 and SEQ ID NO: 58; SEQ ID NO: 38 and SEQ ID NO: 59; SEQ ID NO: 38 and SEQ ID NO: 60; SEQ ID NO: 38 and SEQ ID NO: 61; SEQ ID NO: 38 and SEQ ID NO: 62; SEQ ID NO: 38 and SEQ ID NO: 63; SEQ ID NO: 38 and SEQ ID NO: 64; SEQ ID NO: 38 and SEQ ID NO: 65; SEQ ID NO: 38 and SEQ ID NO: 66; SEQ ID NO: 38 and SEQ ID NO: 67; SEQ ID NO: 38 and SEQ ID NO: 68; SEQ ID NO: 38 and SEQ ID NO: 69; SEQ ID NO: 39 and SEQ ID NO: 40; SEQ ID NO: 39 and SEQ ID NO: 41; SEQ ID NO: 39 and SEQ ID NO: 42; SEQ ID NO: 39 and SEQ ID NO: 43; SEQ ID NO: 39 and SEQ ID NO: 44; SEQ ID NO: 39 and SEQ ID NO: 45; SEQ ID NO: 39 and SEQ ID NO: 46; SEQ ID NO: 39 and SEQ ID NO: 47; SEQ ID NO: 39 and SEQ ID NO: 48; SEQ ID NO: 39 and SEQ ID NO: 53; SEQ ID NO: 39 and SEQ ID NO: 54; SEQ ID NO: 39 and SEQ ID NO: 55; SEQ ID NO: 39 and SEQ ID NO: 56; SEQ ID NO: 39 and SEQ ID NO: 57; SEQ ID NO: 39 and SEQ ID NO: 58; SEQ ID NO: 39 and SEQ ID NO: 59; SEQ ID NO: 39 and SEQ ID NO: 60; SEQ ID NO: 39 and SEQ ID NO: 61; SEQ ID NO: 39 and SEQ ID NO: 62; SEQ ID NO: 39 and SEQ ID NO: 63; SEQ ID NO: 39 and SEQ ID NO: 64; SEQ ID NO: 39 and SEQ ID NO: 65; SEQ ID NO: 39 and SEQ ID NO: 66; SEQ ID NO: 39 and SEQ ID NO: 67; SEQ ID NO: 39 and SEQ ID NO: 68; SEQ ID NO: 39 and SEQ ID NO: 69; SEQ ID NO: 40 and SEQ ID NO: 41; SEQ ID NO: 40 and SEQ ID NO: 42; SEQ ID NO: 40 and SEQ ID NO: 43; SEQ ID NO: 40 and SEQ ID NO: 44; SEQ ID NO: 40 and SEQ ID NO: 45; SEQ ID NO: 40 and SEQ ID NO: 46; SEQ ID NO: 40 and SEQ ID NO: 47; SEQ ID NO: 40 and SEQ ID NO: 48; SEQ ID NO: 40 and SEQ ID NO: 53; SEQ ID NO: 40 and SEQ ID NO: 54; SEQ ID NO: 40 and SEQ ID NO: 55; SEQ ID NO: 40 and SEQ ID NO: 56; SEQ ID NO: 40 and SEQ ID NO: 57; SEQ ID NO: 40 and SEQ ID NO: 58; SEQ ID NO: 40 and SEQ ID NO: 59; SEQ ID NO: 40 and SEQ ID NO: 60; SEQ ID NO: 40 and SEQ ID NO: 61; SEQ ID NO: 40 and SEQ ID NO: 62; SEQ ID NO: 40 and SEQ ID NO: 63; SEQ ID NO: 40 and SEQ ID NO: 64; SEQ ID NO: 40 and SEQ ID NO: 65; SEQ ID NO: 40 and SEQ ID NO: 66; SEQ ID NO: 40 and SEQ ID NO: 67; SEQ ID NO: 40 and SEQ ID NO: 68; SEQ ID NO: 40 and SEQ ID NO: 69; SEQ ID NO: 41 and SEQ ID NO: 42; SEQ ID NO: 41 and SEQ ID NO: 43; SEQ ID NO: 41 and SEQ ID NO: 44; SEQ ID NO: 41 and SEQ ID NO: 45; SEQ ID NO: 41 and SEQ ID NO: 46; SEQ ID NO: 41 and SEQ ID NO: 47; SEQ ID NO: 41 and SEQ ID NO: 48; SEQ ID NO: 41 and SEQ ID NO: 53; SEQ ID NO: 41 and SEQ ID NO: 54; SEQ ID NO: 41 and SEQ ID NO: 55; SEQ ID NO: 41 and SEQ ID NO: 56; SEQ ID NO: 41 and SEQ ID NO: 57; SEQ ID NO: 41 and SEQ ID NO: 58; SEQ ID NO: 41 and SEQ ID NO: 59; SEQ ID NO: 41 and SEQ ID NO: 60; SEQ ID NO: 41 and SEQ ID NO: 61; SEQ ID NO: 41 and SEQ ID NO: 62; SEQ ID NO: 41 and SEQ ID NO: 63; SEQ ID NO: 41 and SEQ ID NO: 64; SEQ ID NO: 41 and SEQ ID NO: 65; SEQ ID NO: 41 and SEQ ID NO: 66; SEQ ID NO: 41 and SEQ ID NO: 67; SEQ ID NO: 41 and SEQ ID NO: 68; SEQ ID NO: 41 and SEQ ID NO: 69; SEQ ID NO: 42 and SEQ ID NO: 43; SEQ ID NO: 42 and SEQ ID NO: 44; SEQ ID NO: 42 and SEQ ID NO: 45; SEQ ID NO: 42 and SEQ ID NO: 46; SEQ ID NO: 42 and SEQ ID NO: 47; SEQ ID NO: 42 and SEQ ID NO: 48; SEQ ID NO: 42 and SEQ ID NO: 53; SEQ ID NO: 42 and SEQ ID NO: 54; SEQ ID NO: 42 and SEQ ID NO: 55; SEQ ID NO: 42 and SEQ ID NO: 56; SEQ ID NO: 42 and SEQ ID NO: 57; SEQ ID NO: 42 and SEQ ID NO: 58; SEQ ID NO: 42 and SEQ ID NO: 59; SEQ ID NO: 42 and SEQ ID NO: 60; SEQ ID NO: 42 and SEQ ID NO: 61; SEQ ID NO: 42 and SEQ ID NO: 62; SEQ ID NO: 42 and SEQ ID NO: 63; SEQ ID NO: 42 and SEQ ID NO: 64; SEQ ID NO: 42 and SEQ ID NO: 65; SEQ ID NO: 42 and SEQ ID NO: 66; SEQ ID NO: 42 and SEQ ID NO: 67; SEQ ID NO: 42 and SEQ ID NO: 68; SEQ ID NO: 42 and SEQ ID NO: 69; SEQ ID NO: 43 and SEQ ID NO: 44; SEQ ID NO: 43 and SEQ ID NO: 45; SEQ ID NO: 43 and SEQ ID NO: 46; SEQ ID NO: 43 and SEQ ID NO: 47; SEQ ID NO: 43 and SEQ ID NO: 48; SEQ ID NO: 43 and SEQ ID NO: 53; SEQ ID NO: 43 and SEQ ID NO: 54; SEQ ID NO: 43 and SEQ ID NO: 55; SEQ ID NO: 43 and SEQ ID NO: 56; SEQ ID NO: 43 and SEQ ID NO: 57; SEQ ID NO: 43 and SEQ ID NO: 58; SEQ ID NO: 43 and SEQ ID NO: 59; SEQ ID NO: 43 and SEQ ID NO: 60; SEQ ID NO: 43 and SEQ ID NO: 61; SEQ ID NO: 43 and SEQ ID NO: 62; SEQ ID NO: 43 and SEQ ID NO: 63; SEQ ID NO: 43 and SEQ ID NO: 64; SEQ ID NO: 43 and SEQ ID NO: 65; SEQ ID NO: 43 and SEQ ID NO: 66; SEQ ID NO: 43 and SEQ ID NO: 67; SEQ ID NO: 43 and SEQ ID NO: 68; SEQ ID NO: 43 and SEQ ID NO: 69; SEQ ID NO: 44 and SEQ ID NO: 45; SEQ ID NO: 44 and SEQ ID NO: 46; SEQ ID NO: 44 and SEQ ID NO: 47; SEQ ID NO: 44 and SEQ ID NO: 48; SEQ ID NO: 44 and SEQ ID NO: 53; SEQ ID NO: 44 and SEQ ID NO: 54; SEQ ID NO: 44 and SEQ ID NO: 55; SEQ ID NO: 44 and SEQ ID NO: 56; SEQ ID NO: 44 and SEQ ID NO: 57; SEQ ID NO: 44 and SEQ ID NO: 58; SEQ ID NO: 44 and SEQ ID NO: 59; SEQ ID NO: 44 and SEQ ID NO: 60; SEQ ID NO: 44 and SEQ ID NO: 61; SEQ ID NO: 44 and SEQ ID NO: 62; SEQ ID NO: 44 and SEQ ID NO: 63; SEQ ID NO: 44 and SEQ ID NO: 64; SEQ ID NO: 44 and SEQ ID NO: 65; SEQ ID NO: 44 and SEQ ID NO: 66; SEQ ID NO: 44 and SEQ ID NO: 67; SEQ ID NO: 44 and SEQ ID NO: 68; SEQ ID NO: 44 and SEQ ID NO: 69; SEQ ID NO: 45 and SEQ ID NO: 46; SEQ ID NO: 45 and SEQ ID NO: 47; SEQ ID NO: 45 and SEQ ID NO: 48; SEQ ID NO: 45 and SEQ ID NO: 53; SEQ ID NO: 45 and SEQ ID NO: 54; SEQ ID NO: 45 and SEQ ID NO: 55; SEQ ID NO: 45 and SEQ ID NO: 56; SEQ ID NO: 45 and SEQ ID NO: 57; SEQ ID NO: 45 and SEQ ID NO: 58; SEQ ID NO: 45 and SEQ ID NO: 59; SEQ ID NO: 45 and SEQ ID NO: 60; SEQ ID NO: 45 and SEQ ID NO: 61; SEQ ID NO: 45 and SEQ ID NO: 62; SEQ ID NO: 45 and SEQ ID NO: 63; SEQ ID NO: 45 and SEQ ID NO: 64; SEQ ID NO: 45 and SEQ ID NO: 65; SEQ ID NO: 45 and SEQ ID NO: 66; SEQ ID NO: 45 and SEQ ID NO: 67; SEQ ID NO: 45 and SEQ ID NO: 68; SEQ ID NO: 45 and SEQ ID NO: 69; SEQ ID NO: 46 and SEQ ID NO: 47; SEQ ID NO: 46 and SEQ ID NO: 48; SEQ ID NO: 46 and SEQ ID NO: 53; SEQ ID NO: 46 and SEQ ID NO: 54; SEQ ID NO: 46 and SEQ ID NO: 55; SEQ ID NO: 46 and SEQ ID NO: 56; SEQ ID NO: 46 and SEQ ID NO: 57; SEQ ID NO: 46 and SEQ ID NO: 58; SEQ ID NO: 46 and SEQ ID NO: 59; SEQ ID NO: 46 and SEQ ID NO: 60; SEQ ID NO: 46 and SEQ ID NO: 61; SEQ ID NO: 46 and SEQ ID NO: 62; SEQ ID NO: 46 and SEQ ID NO: 63; SEQ ID NO: 46 and SEQ ID NO: 64; SEQ ID NO: 46 and SEQ ID NO: 65; SEQ ID NO: 46 and SEQ ID NO: 66; SEQ ID NO: 46 and SEQ ID NO: 67; SEQ ID NO: 46 and SEQ ID NO: 68; SEQ ID NO: 46 and SEQ ID NO: 69; SEQ ID NO: 47 and SEQ ID NO: 48; SEQ ID NO: 47 and SEQ ID NO: 53; SEQ ID NO: 47 and SEQ ID NO: 54; SEQ ID NO: 47 and SEQ ID NO: 55; SEQ ID NO: 47 and SEQ ID NO: 56; SEQ ID NO: 47 and SEQ ID NO: 57; SEQ ID NO: 47 and SEQ ID NO: 58; SEQ ID NO: 47 and SEQ ID NO: 59; SEQ ID NO: 47 and SEQ ID NO: 60; SEQ ID NO: 47 and SEQ ID NO: 61; SEQ ID NO: 47 and SEQ ID NO: 62; SEQ ID NO: 47 and SEQ ID NO: 63; SEQ ID NO: 47 and SEQ ID NO: 64; SEQ ID NO: 47 and SEQ ID NO: 65; SEQ ID NO: 47 and SEQ ID NO: 66; SEQ ID NO: 47 and SEQ ID NO: 67; SEQ ID NO: 47 and SEQ ID NO: 68; SEQ ID NO: 47 and SEQ ID NO: 69; SEQ ID NO: 48 and SEQ ID NO: 53; SEQ ID NO: 48 and SEQ ID NO: 54; SEQ ID NO: 48 and SEQ ID NO: 55; SEQ ID NO: 48 and SEQ ID NO: 56; SEQ ID NO: 48 and SEQ ID NO: 57; SEQ ID NO: 48 and SEQ ID NO: 58; SEQ ID NO: 48 and SEQ ID NO: 59; SEQ ID NO: 48 and SEQ ID NO: 60; SEQ ID NO: 48 and SEQ ID NO: 61; SEQ ID NO: 48 and SEQ ID NO: 62; SEQ ID NO: 48 and SEQ ID NO: 63; SEQ ID NO: 48 and SEQ ID NO: 64; SEQ ID NO: 48 and SEQ ID NO: 65; SEQ ID NO: 48 and SEQ ID NO: 66; SEQ ID NO: 48 and SEQ ID NO: 67; SEQ ID NO: 48 and SEQ ID NO: 68; SEQ ID NO: 48 and SEQ ID NO: 69; SEQ ID NO: 53 and SEQ ID NO: 54; SEQ ID NO: 53 and SEQ ID NO: 55; SEQ ID NO: 53 and SEQ ID NO: 56; SEQ ID NO: 53 and SEQ ID NO: 57; SEQ ID NO: 53 and SEQ ID NO: 58; SEQ ID NO: 53 and SEQ ID NO: 59; SEQ ID NO: 53 and SEQ ID NO: 60; SEQ ID NO: 53 and SEQ ID NO: 61; SEQ ID NO: 53 and SEQ ID NO: 62; SEQ ID NO: 53 and SEQ ID NO: 63; SEQ ID NO: 53 and SEQ ID NO: 64; SEQ ID NO: 53 and SEQ ID NO: 65; SEQ ID NO: 53 and SEQ ID NO: 66; SEQ ID NO: 53 and SEQ ID NO: 67; SEQ ID NO: 53 and SEQ ID NO: 68; SEQ ID NO: 53 and SEQ ID NO: 69; SEQ ID NO: 54 and SEQ ID NO: 55; SEQ ID NO: 54 and SEQ ID NO: 56; SEQ ID NO: 54 and SEQ ID NO: 57; SEQ ID NO: 54 and SEQ ID NO: 58; SEQ ID NO: 54 and SEQ ID NO: 59; SEQ ID NO: 54 and SEQ ID NO: 60; SEQ ID NO: 54 and SEQ ID NO: 61; SEQ ID NO: 54 and SEQ ID NO: 62; SEQ ID NO: 54 and SEQ ID NO: 63; SEQ ID NO: 54 and SEQ ID NO: 64; SEQ ID NO: 54 and SEQ ID NO: 65; SEQ ID NO: 54 and SEQ ID NO: 66; SEQ ID NO: 54 and SEQ ID NO: 67; SEQ ID NO: 54 and SEQ ID NO: 68; SEQ ID NO: 54 and SEQ ID NO: 69; SEQ ID NO: 55 and SEQ ID NO: 56; SEQ ID NO: 55 and SEQ ID NO: 57; SEQ ID NO: 55 and SEQ ID NO: 58; SEQ ID NO: 55 and SEQ ID NO: 59; SEQ ID NO: 55 and SEQ ID NO: 60; SEQ ID NO: 55 and SEQ ID NO: 61; SEQ ID NO: 55 and SEQ ID NO: 62; SEQ ID NO: 55 and SEQ ID NO: 63; SEQ ID NO: 55 and SEQ ID NO: 64; SEQ ID NO: 55 and SEQ ID NO: 65; SEQ ID NO: 55 and SEQ ID NO: 66; SEQ ID NO: 55 and SEQ ID NO: 67; SEQ ID NO: 55 and SEQ ID NO: 68; SEQ ID NO: 55 and SEQ ID NO: 69; SEQ ID NO: 56 and SEQ ID NO: 57; SEQ ID NO: 56 and SEQ ID NO: 58; SEQ ID NO: 56 and SEQ ID NO: 59; SEQ ID NO: 56 and SEQ ID NO: 60; SEQ ID NO: 56 and SEQ ID NO: 61; SEQ ID NO: 56 and SEQ ID NO: 62; SEQ ID NO: 56 and SEQ ID NO: 63; SEQ ID NO: 56 and SEQ ID NO: 64; SEQ ID NO: 56 and SEQ ID NO: 65; SEQ ID NO: 56 and SEQ ID NO: 66; SEQ ID NO: 56 and SEQ ID NO: 67; SEQ ID NO: 56 and SEQ ID NO: 68; SEQ ID NO: 56 and SEQ ID NO: 69; SEQ ID NO: 57 and SEQ ID NO: 58; SEQ ID NO: 57 and SEQ ID NO: 59; SEQ ID NO: 57 and SEQ ID NO: 60; SEQ ID NO: 57 and SEQ ID NO: 61; SEQ ID NO: 57 and SEQ ID NO: 62; SEQ ID NO: 57 and SEQ ID NO: 63; SEQ ID NO: 57 and SEQ ID NO: 64; SEQ ID NO: 57 and SEQ ID NO: 65; SEQ ID NO: 57 and SEQ ID NO: 66; SEQ ID NO: 57 and SEQ ID NO: 67; SEQ ID NO: 57 and SEQ ID NO: 68; SEQ ID NO: 57 and SEQ ID NO: 69; SEQ ID NO: 58 and SEQ ID NO: 59; SEQ ID NO: 58 and SEQ ID NO: 60; SEQ ID NO: 58 and SEQ ID NO: 61; SEQ ID NO: 58 and SEQ ID NO: 62; SEQ ID NO: 58 and SEQ ID NO: 63; SEQ ID NO: 58 and SEQ ID NO: 64; SEQ ID NO: 58 and SEQ ID NO: 65; SEQ ID NO: 58 and SEQ ID NO: 66; SEQ ID NO: 58 and SEQ ID NO: 67; SEQ ID NO: 58 and SEQ ID NO: 68; SEQ ID NO: 58 and SEQ ID NO: 69; SEQ ID NO: 59 and SEQ ID NO: 60; SEQ ID NO: 59 and SEQ ID NO: 61; SEQ ID NO: 59 and SEQ ID NO: 62; SEQ ID NO: 59 and SEQ ID NO: 63; SEQ ID NO: 59 and SEQ ID NO: 64; SEQ ID NO: 59 and SEQ ID NO: 65; SEQ ID NO: 59 and SEQ ID NO: 66; SEQ ID NO: 59 and SEQ ID NO: 67; SEQ ID NO: 59 and SEQ ID NO: 68; SEQ ID NO: 59 and SEQ ID NO: 69; SEQ ID NO: 60 and SEQ ID NO: 61; SEQ ID NO: 60 and SEQ ID NO: 62; SEQ ID NO: 60 and SEQ ID NO: 63; SEQ ID NO: 60 and SEQ ID NO: 64; SEQ ID NO: 60 and SEQ ID NO: 65; SEQ ID NO: 60 and SEQ ID NO: 66; SEQ ID NO: 60 and SEQ ID NO: 67; SEQ ID NO: 60 and SEQ ID NO: 68; SEQ ID NO: 60 and SEQ ID NO: 69; SEQ ID NO: 61 and SEQ ID NO: 62; SEQ ID NO: 61 and SEQ ID NO: 63; SEQ ID NO: 61 and SEQ ID NO: 64; SEQ ID NO: 61 and SEQ ID NO: 65; SEQ ID NO: 61 and SEQ ID NO: 66; SEQ ID NO: 61 and SEQ ID NO: 67; SEQ ID NO: 61 and SEQ ID NO: 68; SEQ ID NO: 61 and SEQ ID NO: 69; SEQ ID NO: 62 and SEQ ID NO: 63; SEQ ID NO: 62 and SEQ ID NO: 64; SEQ ID NO: 62 and SEQ ID NO: 65; SEQ ID NO: 62 and SEQ ID NO: 66; SEQ ID NO: 62 and SEQ ID NO: 67; SEQ ID NO: 62 and SEQ ID NO: 68; SEQ ID NO: 62 and SEQ ID NO: 69; SEQ ID NO: 63 and SEQ ID NO: 64; SEQ ID NO: 63 and SEQ ID NO: 65; SEQ ID NO: 63 and SEQ ID NO: 66; SEQ ID NO: 63 and SEQ ID NO: 67; SEQ ID NO: 63 and SEQ ID NO: 68; SEQ ID NO: 63 and SEQ ID NO: 69; SEQ ID NO: 64 and SEQ ID NO: 65; SEQ ID NO: 64 and SEQ ID NO: 66; SEQ ID NO: 64 and SEQ ID NO: 67; SEQ ID NO: 64 and SEQ ID NO: 68; SEQ ID NO: 64 and SEQ ID NO: 69; SEQ ID NO: 65 and SEQ ID NO: 66; SEQ ID NO: 65 and SEQ ID NO: 67; SEQ ID NO: 65 and SEQ ID NO: 68; SEQ ID NO: 65 and SEQ ID NO: 69; SEQ ID NO: 66 and SEQ ID NO: 67; SEQ ID NO: 66 and SEQ ID NO: 68; SEQ ID NO: 66 and SEQ ID NO: 69; SEQ ID NO: 67 and SEQ ID NO: 68; SEQ ID NO: 67 and SEQ ID NO: 69; SEQ ID NO: 68 and SEQ ID NO: 69; SEQ ID NO: 71 and SEQ ID NO: 72; SEQ ID NO: 71 and SEQ ID NO: 73; SEQ ID NO: 71 and SEQ ID NO: 74; SEQ ID NO: 71 and SEQ ID NO: 75; SEQ ID NO: 71 and SEQ ID NO: 76; SEQ ID NO: 71 and SEQ ID NO: 77; SEQ ID NO: 71 and SEQ ID NO: 78; SEQ ID NO: 71 and SEQ ID NO: 84; SEQ ID NO: 71 and SEQ ID NO: 85; SEQ ID NO: 71 and SEQ ID NO: 86; SEQ ID NO: 71 and SEQ ID NO: 87; SEQ ID NO: 71 and SEQ ID NO: 88; SEQ ID NO: 71 and SEQ ID NO: 89; SEQ ID NO: 71 and SEQ ID NO: 90; SEQ ID NO: 71 and SEQ ID NO: 91; SEQ ID NO: 71 and SEQ ID NO: 92; SEQ ID NO: 71 and SEQ ID NO: 93; SEQ ID NO: 71 and SEQ ID NO: 94; SEQ ID NO: 71 and SEQ ID NO: 95; SEQ ID NO: 71 and SEQ ID NO: 96; SEQ ID NO: 71 and SEQ ID NO: 97; SEQ ID NO: 71 and SEQ ID NO: 98; SEQ ID NO: 71 and SEQ ID NO: 99; SEQ ID NO: 71 and SEQ ID NO: 100; SEQ ID NO: 71 and SEQ ID NO: 101; SEQ ID NO: 71 and SEQ ID NO: 102; SEQ ID NO: 72 and SEQ ID NO: 73; SEQ ID NO: 72 and SEQ ID NO: 74; SEQ ID NO: 72 and SEQ ID NO: 75; SEQ ID NO: 72 and SEQ ID NO: 76; SEQ ID NO: 72 and SEQ ID NO: 77; SEQ ID NO: 72 and SEQ ID NO: 78; SEQ ID NO: 72 and SEQ ID NO: 84; SEQ ID NO: 72 and SEQ ID NO: 85; SEQ ID NO: 72 and SEQ ID NO: 86; SEQ ID NO: 72 and SEQ ID NO: 87; SEQ ID NO: 72 and SEQ ID NO: 88; SEQ ID NO: 72 and SEQ ID NO: 89; SEQ ID NO: 72 and SEQ ID NO: 90; SEQ ID NO: 72 and SEQ ID NO: 91; SEQ ID NO: 72 and SEQ ID NO: 92; SEQ ID NO: 72 and SEQ ID NO: 93; SEQ ID NO: 72 and SEQ ID NO: 94; SEQ ID NO: 72 and SEQ ID NO: 95; SEQ ID NO: 72 and SEQ ID NO: 96; SEQ ID NO: 72 and SEQ ID NO: 97; SEQ ID NO: 72 and SEQ ID NO: 98; SEQ ID NO: 72 and SEQ ID NO: 99; SEQ ID NO: 72 and SEQ ID NO: 100; SEQ ID NO: 72 and SEQ ID NO: 101; SEQ ID NO: 72 and SEQ ID NO: 102; SEQ ID NO: 73 and SEQ ID NO: 74; SEQ ID NO: 73 and SEQ ID NO: 75; SEQ ID NO: 73 and SEQ ID NO: 76; SEQ ID NO: 73 and SEQ ID NO: 77; SEQ ID NO: 73 and SEQ ID NO: 78; SEQ ID NO: 73 and SEQ ID NO: 84; SEQ ID NO: 73 and SEQ ID NO: 85; SEQ ID NO: 73 and SEQ ID NO: 86; SEQ ID NO: 73 and SEQ ID NO: 87; SEQ ID NO: 73 and SEQ ID NO: 88; SEQ ID NO: 73 and SEQ ID NO: 89; SEQ ID NO: 73 and SEQ ID NO: 90; SEQ ID NO: 73 and SEQ ID NO: 91; SEQ ID NO: 73 and SEQ ID NO: 92; SEQ ID NO: 73 and SEQ ID NO: 93; SEQ ID NO: 73 and SEQ ID NO: 94; SEQ ID NO: 73 and SEQ ID NO: 95; SEQ ID NO: 73 and SEQ ID NO: 96; SEQ ID NO: 73 and SEQ ID NO: 97; SEQ ID NO: 73 and SEQ ID NO: 98; SEQ ID NO: 73 and SEQ ID NO: 99; SEQ ID NO: 73 and SEQ ID NO: 100; SEQ ID NO: 73 and SEQ ID NO: 101; SEQ ID NO: 73 and SEQ ID NO: 102; SEQ ID NO: 74 and SEQ ID NO: 75; SEQ ID NO: 74 and SEQ ID NO: 76; SEQ ID NO: 74 and SEQ ID NO: 77; SEQ ID NO: 74 and SEQ ID NO: 78; SEQ ID NO: 74 and SEQ ID NO: 84; SEQ ID NO: 74 and SEQ ID NO: 85; SEQ ID NO: 74 and SEQ ID NO: 86; SEQ ID NO: 74 and SEQ ID NO: 87; SEQ ID NO: 74 and SEQ ID NO: 88; SEQ ID NO: 74 and SEQ ID NO: 89; SEQ ID NO: 74 and SEQ ID NO: 90; SEQ ID NO: 74 and SEQ ID NO: 91; SEQ ID NO: 74 and SEQ ID NO: 92; SEQ ID NO: 74 and SEQ ID NO: 93; SEQ ID NO: 74 and SEQ ID NO: 94; SEQ ID NO: 74 and SEQ ID NO: 95; SEQ ID NO: 74 and SEQ ID NO: 96; SEQ ID NO: 74 and SEQ ID NO: 97; SEQ ID NO: 74 and SEQ ID NO: 98; SEQ ID NO: 74 and SEQ ID NO: 99; SEQ ID NO: 74 and SEQ ID NO: 100; SEQ ID NO: 74 and SEQ ID NO: 101; SEQ ID NO: 74 and SEQ ID NO: 102; SEQ ID NO: 75 and SEQ ID NO: 76; SEQ ID NO: 75 and SEQ ID NO: 77; SEQ ID NO: 75 and SEQ ID NO: 78; SEQ ID NO: 75 and SEQ ID NO: 84; SEQ ID NO: 75 and SEQ ID NO: 85; SEQ ID NO: 75 and SEQ ID NO: 86; SEQ ID NO: 75 and SEQ ID NO: 87; SEQ ID NO: 75 and SEQ ID NO: 88; SEQ ID NO: 75 and SEQ ID NO: 89; SEQ ID NO: 75 and SEQ ID NO: 90; SEQ ID NO: 75 and SEQ ID NO: 91; SEQ ID NO: 75 and SEQ ID NO: 92; SEQ ID NO: 75 and SEQ ID NO: 93; SEQ ID NO: 75 and SEQ ID NO: 94; SEQ ID NO: 75 and SEQ ID NO: 95; SEQ ID NO: 75 and SEQ ID NO: 96; SEQ ID NO: 75 and SEQ ID NO: 97; SEQ ID NO: 75 and SEQ ID NO: 98; SEQ ID NO: 75 and SEQ ID NO: 99; SEQ ID NO: 75 and SEQ ID NO: 100; SEQ ID NO: 75 and SEQ ID NO: 101; SEQ ID NO: 75 and SEQ ID NO: 102; SEQ ID NO: 76 and SEQ ID NO: 77; SEQ ID NO: 76 and SEQ ID NO: 78; SEQ ID NO: 76 and SEQ ID NO: 84; SEQ ID NO: 76 and SEQ ID NO: 85; SEQ ID NO: 76 and SEQ ID NO: 86; SEQ ID NO: 76 and SEQ ID NO: 87; SEQ ID NO: 76 and SEQ ID NO: 88; SEQ ID NO: 76 and SEQ ID NO: 89; SEQ ID NO: 76 and SEQ ID NO: 90; SEQ ID NO: 76 and SEQ ID NO: 91; SEQ ID NO: 76 and SEQ ID NO: 92; SEQ ID NO: 76 and SEQ ID NO: 93; SEQ ID NO: 76 and SEQ ID NO: 94; SEQ ID NO: 76 and SEQ ID NO: 95; SEQ ID NO: 76 and SEQ ID NO: 96; SEQ ID NO: 76 and SEQ ID NO: 97; SEQ ID NO: 76 and SEQ ID NO: 98; SEQ ID NO: 76 and SEQ ID NO: 99; SEQ ID NO: 76 and SEQ ID NO: 100; SEQ ID NO: 76 and SEQ ID NO: 101; SEQ ID NO: 76 and SEQ ID NO: 102; SEQ ID NO: 77 and SEQ ID NO: 78; SEQ ID NO: 77 and SEQ ID NO: 84; SEQ ID NO: 77 and SEQ ID NO: 85; SEQ ID NO: 77 and SEQ ID NO: 86; SEQ ID NO: 77 and SEQ ID NO: 87; SEQ ID NO: 77 and SEQ ID NO: 88; SEQ ID NO: 77 and SEQ ID NO: 89; SEQ ID NO: 77 and SEQ ID NO: 90; SEQ ID NO: 77 and SEQ ID NO: 91; SEQ ID NO: 77 and SEQ ID NO: 92; SEQ ID NO: 77 and SEQ ID NO: 93; SEQ ID NO: 77 and SEQ ID NO: 94; SEQ ID NO: 77 and SEQ ID NO: 95; SEQ ID NO: 77 and SEQ ID NO: 96; SEQ ID NO: 77 and SEQ ID NO: 97; SEQ ID NO: 77 and SEQ ID NO: 98; SEQ ID NO: 77 and SEQ ID NO: 99; SEQ ID NO: 77 and SEQ ID NO: 100; SEQ ID NO: 77 and SEQ ID NO: 101; SEQ ID NO: 77 and SEQ ID NO: 102; SEQ ID NO: 78 and SEQ ID NO: 84; SEQ ID NO: 78 and SEQ ID NO: 85; SEQ ID NO: 78 and SEQ ID NO: 86; SEQ ID NO: 78 and SEQ ID NO: 87; SEQ ID NO: 78 and SEQ ID NO: 88; SEQ ID NO: 78 and SEQ ID NO: 89; SEQ ID NO: 78 and SEQ ID NO: 90; SEQ ID NO: 78 and SEQ ID NO: 91; SEQ ID NO: 78 and SEQ ID NO: 92; SEQ ID NO: 78 and SEQ ID NO: 93; SEQ ID NO: 78 and SEQ ID NO: 94; SEQ ID NO: 78 and SEQ ID NO: 95; SEQ ID NO: 78 and SEQ ID NO: 96; SEQ ID NO: 78 and SEQ ID NO: 97; SEQ ID NO: 78 and SEQ ID NO: 98; SEQ ID NO: 78 and SEQ ID NO: 99; SEQ ID NO: 78 and SEQ ID NO: 100; SEQ ID NO: 78 and SEQ ID NO: 101; SEQ ID NO: 78 and SEQ ID NO: 102; SEQ ID NO: 84 and SEQ ID NO: 85; SEQ ID NO: 84 and SEQ ID NO: 86; SEQ ID NO: 84 and SEQ ID NO: 87; SEQ ID NO: 84 and SEQ ID NO: 88; SEQ ID NO: 84 and SEQ ID NO: 89; SEQ ID NO: 84 and SEQ ID NO: 90; SEQ ID NO: 84 and SEQ ID NO: 91; SEQ ID NO: 84 and SEQ ID NO: 92; SEQ ID NO: 84 and SEQ ID NO: 93; SEQ ID NO: 84 and SEQ ID NO: 94; SEQ ID NO: 84 and SEQ ID NO: 95; SEQ ID NO: 84 and SEQ ID NO: 96; SEQ ID NO: 84 and SEQ ID NO: 97; SEQ ID NO: 84 and SEQ ID NO: 98; SEQ ID NO: 84 and SEQ ID NO: 99; SEQ ID NO: 84 and SEQ ID NO: 100; SEQ ID NO: 84 and SEQ ID NO: 101; SEQ ID NO: 84 and SEQ ID NO: 102; SEQ ID NO: 85 and SEQ ID NO: 86; SEQ ID NO: 85 and SEQ ID NO: 87; SEQ ID NO: 85 and SEQ ID NO: 88; SEQ ID NO: 85 and SEQ ID NO: 89; SEQ ID NO: 85 and SEQ ID NO: 90; SEQ ID NO: 85 and SEQ ID NO: 91; SEQ ID NO: 85 and SEQ ID NO: 92; SEQ ID NO: 85 and SEQ ID NO: 93; SEQ ID NO: 85 and SEQ ID NO: 94; SEQ ID NO: 85 and SEQ ID NO: 95; SEQ ID NO: 85 and SEQ ID NO: 96; SEQ ID NO: 85 and SEQ ID NO: 97; SEQ ID NO: 85 and SEQ ID NO: 98; SEQ ID NO: 85 and SEQ ID NO: 99; SEQ ID NO: 85 and SEQ ID NO: 100; SEQ ID NO: 85 and SEQ ID NO: 101; SEQ ID NO: 85 and SEQ ID NO: 102; SEQ ID NO: 86 and SEQ ID NO: 87; SEQ ID NO: 86 and SEQ ID NO: 88; SEQ ID NO: 86 and SEQ ID NO: 89; SEQ ID NO: 86 and SEQ ID NO: 90; SEQ ID NO: 86 and SEQ ID NO: 91; SEQ ID NO: 86 and SEQ ID NO: 92; SEQ ID NO: 86 and SEQ ID NO: 93; SEQ ID NO: 86 and SEQ ID NO: 94; SEQ ID NO: 86 and SEQ ID NO: 95; SEQ ID NO: 86 and SEQ ID NO: 96; SEQ ID NO: 86 and SEQ ID NO: 97; SEQ ID NO: 86 and SEQ ID NO: 98; SEQ ID NO: 86 and SEQ ID NO: 99; SEQ ID NO: 86 and SEQ ID NO: 100; SEQ ID NO: 86 and SEQ ID NO: 101; SEQ ID NO: 86 and SEQ ID NO: 102; SEQ ID NO: 87 and SEQ ID NO: 88; SEQ ID NO: 87 and SEQ ID NO: 89; SEQ ID NO: 87 and SEQ ID NO: 90; SEQ ID NO: 87 and SEQ ID NO: 91; SEQ ID NO: 87 and SEQ ID NO: 92; SEQ ID NO: 87 and SEQ ID NO: 93; SEQ ID NO: 87 and SEQ ID NO: 94; SEQ ID NO: 87 and SEQ ID NO: 95; SEQ ID NO: 87 and SEQ ID NO: 96; SEQ ID NO: 87 and SEQ ID NO: 97; SEQ ID NO: 87 and SEQ ID NO: 98; SEQ ID NO: 87 and SEQ ID NO: 99; SEQ ID NO: 87 and SEQ ID NO: 100; SEQ ID NO: 87 and SEQ ID NO: 101; SEQ ID NO: 87 and SEQ ID NO: 102; SEQ ID NO: 88 and SEQ ID NO: 89; SEQ ID NO: 88 and SEQ ID NO: 90; SEQ ID NO: 88 and SEQ ID NO: 91; SEQ ID NO: 88 and SEQ ID NO: 92; SEQ ID NO: 88 and SEQ ID NO: 93; SEQ ID NO: 88 and SEQ ID NO: 94; SEQ ID NO: 88 and SEQ ID NO: 95; SEQ ID NO: 88 and SEQ ID NO: 96; SEQ ID NO: 88 and SEQ ID NO: 97; SEQ ID NO: 88 and SEQ ID NO: 98; SEQ ID NO: 88 and SEQ ID NO: 99; SEQ ID NO: 88 and SEQ ID NO: 100; SEQ ID NO: 88 and SEQ ID NO: 101; SEQ ID NO: 88 and SEQ ID NO: 102; SEQ ID NO: 89 and SEQ ID NO: 90; SEQ ID NO: 89 and SEQ ID NO: 91; SEQ ID NO: 89 and SEQ ID NO: 92; SEQ ID NO: 89 and SEQ ID NO: 93; SEQ ID NO: 89 and SEQ ID NO: 94; SEQ ID NO: 89 and SEQ ID NO: 95; SEQ ID NO: 89 and SEQ ID NO: 96; SEQ ID NO: 89 and SEQ ID NO: 97; SEQ ID NO: 89 and SEQ ID NO: 98; SEQ ID NO: 89 and SEQ ID NO: 99; SEQ ID NO: 89 and SEQ ID NO: 100; SEQ ID NO: 89 and SEQ ID NO: 101; SEQ ID NO: 89 and SEQ ID NO: 102; SEQ ID NO: 90 and SEQ ID NO: 91; SEQ ID NO: 90 and SEQ ID NO: 92; SEQ ID NO: 90 and SEQ ID NO: 93; SEQ ID NO: 90 and SEQ ID NO: 94; SEQ ID NO: 90 and SEQ ID NO: 95; SEQ ID NO: 90 and SEQ ID NO: 96; SEQ ID NO: 90 and SEQ ID NO: 97; SEQ ID NO: 90 and SEQ ID NO: 98; SEQ ID NO: 90 and SEQ ID NO: 99; SEQ ID NO: 90 and SEQ ID NO: 100; SEQ ID NO: 90 and SEQ ID NO: 101; SEQ ID NO: 90 and SEQ ID NO: 102; SEQ ID NO: 91 and SEQ ID NO: 92; SEQ ID NO: 91 and SEQ ID NO: 93; SEQ ID NO: 91 and SEQ ID NO: 94; SEQ ID NO: 91 and SEQ ID NO: 95; SEQ ID NO: 91 and SEQ ID NO: 96; SEQ ID NO: 91 and SEQ ID NO: 97; SEQ ID NO: 91 and SEQ ID NO: 98; SEQ ID NO: 91 and SEQ ID NO: 99; SEQ ID NO: 91 and SEQ ID NO: 100; SEQ ID NO: 91 and SEQ ID NO: 101; SEQ ID NO: 91 and SEQ ID NO: 102; SEQ ID NO: 92 and SEQ ID NO: 93; SEQ ID NO: 92 and SEQ ID NO: 94; SEQ ID NO: 92 and SEQ ID NO: 95; SEQ ID NO: 92 and SEQ ID NO: 96; SEQ ID NO: 92 and SEQ ID NO: 97; SEQ ID NO: 92 and SEQ ID NO: 98; SEQ ID NO: 92 and SEQ ID NO: 99; SEQ ID NO: 92 and SEQ ID NO: 100; SEQ ID NO: 92 and SEQ ID NO: 101; SEQ ID NO: 92 and SEQ ID NO: 102; SEQ ID NO: 93 and SEQ ID NO: 94; SEQ ID NO: 93 and SEQ ID NO: 95; SEQ ID NO: 93 and SEQ ID NO: 96; SEQ ID NO: 93 and SEQ ID NO: 97; SEQ ID NO: 93 and SEQ ID NO: 98; SEQ ID NO: 93 and SEQ ID NO: 99; SEQ ID NO: 93 and SEQ ID NO: 100; SEQ ID NO: 93 and SEQ ID NO: 101; SEQ ID NO: 93 and SEQ ID NO: 102; SEQ ID NO: 94 and SEQ ID NO: 95; SEQ ID NO: 94 and SEQ ID NO: 96; SEQ ID NO: 94 and SEQ ID NO: 97; SEQ ID NO: 94 and SEQ ID NO: 98; SEQ ID NO: 94 and SEQ ID NO: 99; SEQ ID NO: 94 and SEQ ID NO: 100; SEQ ID NO: 94 and SEQ ID NO: 101; SEQ ID NO: 94 and SEQ ID NO: 102; SEQ ID NO: 95 and SEQ ID NO: 96; SEQ ID NO: 95 and SEQ ID NO: 97; SEQ ID NO: 95 and SEQ ID NO: 98; SEQ ID NO: 95 and SEQ ID NO: 99; SEQ ID NO: 95 and SEQ ID NO: 100; SEQ ID NO: 95 and SEQ ID NO: 101; SEQ ID NO: 95 and SEQ ID NO: 102; SEQ ID NO: 96 and SEQ ID NO: 97; SEQ ID NO: 96 and SEQ ID NO: 98; SEQ ID NO: 96 and SEQ ID NO: 99; SEQ ID NO: 96 and SEQ ID NO: 100; SEQ ID NO: 96 and SEQ ID NO: 101; SEQ ID NO: 96 and SEQ ID NO: 102; SEQ ID NO: 97 and SEQ ID NO: 98; SEQ ID NO: 97 and SEQ ID NO: 99; SEQ ID NO: 97 and SEQ ID NO: 100; SEQ ID NO: 97 and SEQ ID NO: 101; SEQ ID NO: 97 and SEQ ID NO: 102; SEQ ID NO: 98 and SEQ ID NO: 99; SEQ ID NO: 98 and SEQ ID NO: 100; SEQ ID NO: 98 and SEQ ID NO: 101; SEQ ID NO: 98 and SEQ ID NO: 102; SEQ ID NO: 99 and SEQ ID NO: 100; SEQ ID NO: 99 and SEQ ID NO: 101; SEQ ID NO: 99 and SEQ ID NO: 102; SEQ ID NO: 100 and SEQ ID NO: 101; SEQ ID NO: 100 and SEQ ID NO: 102; SEQ ID NO: 101 and SEQ ID NO: 102; SEQ ID NO: 200 and SEQ ID NO: 201; SEQ ID NO: 200 and SEQ ID NO: 202; SEQ ID NO: 200 and SEQ ID NO: 203; SEQ ID NO: 200 and SEQ ID NO: 204; SEQ ID NO: 200 and SEQ ID NO: 205; SEQ ID NO: 200 and SEQ ID NO: 206; SEQ ID NO: 200 and SEQ ID NO: 207; SEQ ID NO: 200 and SEQ ID NO: 208; SEQ ID NO: 201 and SEQ ID NO: 202; SEQ ID NO: 201 and SEQ ID NO: 203; SEQ ID NO: 201 and SEQ ID NO: 204; SEQ ID NO: 201 and SEQ ID NO: 205; SEQ ID NO: 201 and SEQ ID NO: 206; SEQ ID NO: 201 and SEQ ID NO: 207; SEQ ID NO: 201 and SEQ ID NO: 208; SEQ ID NO: 202 and SEQ ID NO: 203; SEQ ID NO: 202 and SEQ ID NO: 204; SEQ ID NO: 202 and SEQ ID NO: 205; SEQ ID NO: 202 and SEQ ID NO: 206; SEQ ID NO: 202 and SEQ ID NO: 207; SEQ ID NO: 202 and SEQ ID NO: 208; SEQ ID NO: 203 and SEQ ID NO: 204; SEQ ID NO: 203 and SEQ ID NO: 205; SEQ ID NO: 203 and SEQ ID NO: 206; SEQ ID NO: 203 and SEQ ID NO: 207; SEQ ID NO: 203 and SEQ ID NO: 208; SEQ ID NO: 204 and SEQ ID NO: 205; SEQ ID NO: 204 and SEQ ID NO: 206; SEQ ID NO: 204 and SEQ ID NO: 207; SEQ ID NO: 204 and SEQ ID NO: 208; SEQ ID NO: 205 and SEQ ID NO: 206; SEQ ID NO: 205 and SEQ ID NO: 207; SEQ ID NO: 205 and SEQ ID NO: 208; SEQ ID NO: 206 and SEQ ID NO: 207; SEQ ID NO: 206 and SEQ ID NO: 208; SEQ ID NO: 207 and SEQ ID NO: 208; SEQ ID NO: 209 and SEQ ID NO: 210; SEQ ID NO: 209 and SEQ ID NO: 211; SEQ ID NO: 209 and SEQ ID NO: 212; SEQ ID NO: 209 and SEQ ID NO: 213; SEQ ID NO: 209 and SEQ ID NO: 214; SEQ ID NO: 209 and SEQ ID NO: 215; SEQ ID NO: 209 and SEQ ID NO: 216; SEQ ID NO: 209 and SEQ ID NO: 217; SEQ ID NO: 209 and SEQ ID NO: 218; SEQ ID NO: 209 and SEQ ID NO: 219; SEQ ID NO: 209 and SEQ ID NO: 220; SEQ ID NO: 209 and SEQ ID NO: 221; SEQ ID NO: 209 and SEQ ID NO: 222; SEQ ID NO: 209 and SEQ ID NO: 223; SEQ ID NO: 209 and SEQ ID NO: 224; SEQ ID NO: 209 and SEQ ID NO: 225; SEQ ID NO: 209 and SEQ ID NO: 226; SEQ ID NO: 209 and SEQ ID NO: 227; SEQ ID NO: 209 and SEQ ID NO: 228; SEQ ID NO: 209 and SEQ ID NO: 229; SEQ ID NO: 209 and SEQ ID NO: 230; SEQ ID NO: 210 and SEQ ID NO: 211; SEQ ID NO: 210 and SEQ ID NO: 212; SEQ ID NO: 210 and SEQ ID NO: 213; SEQ ID NO: 210 and SEQ ID NO: 214; SEQ ID NO: 210 and SEQ ID NO: 215; SEQ ID NO: 210 and SEQ ID NO: 216; SEQ ID NO: 210 and SEQ ID NO: 217; SEQ ID NO: 210 and SEQ ID NO: 218; SEQ ID NO: 210 and SEQ ID NO: 219; SEQ ID NO: 210 and SEQ ID NO: 220; SEQ ID NO: 210 and SEQ ID NO: 221; SEQ ID NO: 210 and SEQ ID NO: 222; SEQ ID NO: 210 and SEQ ID NO: 223; SEQ ID NO: 210 and SEQ ID NO: 224; SEQ ID NO: 210 and SEQ ID NO: 225; SEQ ID NO: 210 and SEQ ID NO: 226; SEQ ID NO: 210 and SEQ ID NO: 227; SEQ ID NO: 210 and SEQ ID NO: 228; SEQ ID NO: 210 and SEQ ID NO: 229; SEQ ID NO: 210 and SEQ ID NO: 230; SEQ ID NO: 211 and SEQ ID NO: 212; SEQ ID NO: 211 and SEQ ID NO: 213; SEQ ID NO: 211 and SEQ ID NO: 214; SEQ ID NO: 211 and SEQ ID NO: 215; SEQ ID NO: 211 and SEQ ID NO: 216; SEQ ID NO: 211 and SEQ ID NO: 217; SEQ ID NO: 211 and SEQ ID NO: 218; SEQ ID NO: 211 and SEQ ID NO: 219; SEQ ID NO: 211 and SEQ ID NO: 220; SEQ ID NO: 211 and SEQ ID NO: 221; SEQ ID NO: 211 and SEQ ID NO: 222; SEQ ID NO: 211 and SEQ ID NO: 223; SEQ ID NO: 211 and SEQ ID NO: 224; SEQ ID NO: 211 and SEQ ID NO: 225; SEQ ID NO: 211 and SEQ ID NO: 226; SEQ ID NO: 211 and SEQ ID NO: 227; SEQ ID NO: 211 and SEQ ID NO: 228; SEQ ID NO: 211 and SEQ ID NO: 229; SEQ ID NO: 211 and SEQ ID NO: 230; SEQ ID NO: 212 and SEQ ID NO: 213; SEQ ID NO: 212 and SEQ ID NO: 214; SEQ ID NO: 212 and SEQ ID NO: 215; SEQ ID NO: 212 and SEQ ID NO: 216; SEQ ID NO: 212 and SEQ ID NO: 217; SEQ ID NO: 212 and SEQ ID NO: 218; SEQ ID NO: 212 and SEQ ID NO: 219; SEQ ID NO: 212 and SEQ ID NO: 220; SEQ ID NO: 212 and SEQ ID NO: 221; SEQ ID NO: 212 and SEQ ID NO: 222; SEQ ID NO: 212 and SEQ ID NO: 223; SEQ ID NO: 212 and SEQ ID NO: 224; SEQ ID NO: 212 and SEQ ID NO: 225; SEQ ID NO: 212 and SEQ ID NO: 226; SEQ ID NO: 212 and SEQ ID NO: 227; SEQ ID NO: 212 and SEQ ID NO: 228; SEQ ID NO: 212 and SEQ ID NO: 229; SEQ ID NO: 212 and SEQ ID NO: 230; SEQ ID NO: 213 and SEQ ID NO: 214; SEQ ID NO: 213 and SEQ ID NO: 215; SEQ ID NO: 213 and SEQ ID NO: 216; SEQ ID NO: 213 and SEQ ID NO: 217; SEQ ID NO: 213 and SEQ ID NO: 218; SEQ ID NO: 213 and SEQ ID NO: 219; SEQ ID NO: 213 and SEQ ID NO: 220; SEQ ID NO: 213 and SEQ ID NO: 221; SEQ ID NO: 213 and SEQ ID NO: 222; SEQ ID NO: 213 and SEQ ID NO: 223; SEQ ID NO: 213 and SEQ ID NO: 224; SEQ ID NO: 213 and SEQ ID NO: 225; SEQ ID NO: 213 and SEQ ID NO: 226; SEQ ID NO: 213 and SEQ ID NO: 227; SEQ ID NO: 213 and SEQ ID NO: 228; SEQ ID NO: 213 and SEQ ID NO: 229; SEQ ID NO: 213 and SEQ ID NO: 230; SEQ ID NO: 214 and SEQ ID NO: 215; SEQ ID NO: 214 and SEQ ID NO: 216; SEQ ID NO: 214 and SEQ ID NO: 217; SEQ ID NO: 214 and SEQ ID NO: 218; SEQ ID NO: 214 and SEQ ID NO: 219; SEQ ID NO: 214 and SEQ ID NO: 220; SEQ ID NO: 214 and SEQ ID NO: 221; SEQ ID NO: 214 and SEQ ID NO: 222; SEQ ID NO: 214 and SEQ ID NO: 223; SEQ ID NO: 214 and SEQ ID NO: 224; SEQ ID NO: 214 and SEQ ID NO: 225; SEQ ID NO: 214 and SEQ ID NO: 226; SEQ ID NO: 214 and SEQ ID NO: 227; SEQ ID NO: 214 and SEQ ID NO: 228; SEQ ID NO: 214 and SEQ ID NO: 229; SEQ ID NO: 214 and SEQ ID NO: 230; SEQ ID NO: 215 and SEQ ID NO: 216; SEQ ID NO: 215 and SEQ ID NO: 217; SEQ ID NO: 215 and SEQ ID NO: 218; SEQ ID NO: 215 and SEQ ID NO: 219; SEQ ID NO: 215 and SEQ ID NO: 220; SEQ ID NO: 215 and SEQ ID NO: 221; SEQ ID NO: 215 and SEQ ID NO: 222; SEQ ID NO: 215 and SEQ ID NO: 223; SEQ ID NO: 215 and SEQ ID NO: 224; SEQ ID NO: 215 and SEQ ID NO: 225; SEQ ID NO: 215 and SEQ ID NO: 226; SEQ ID NO: 215 and SEQ ID NO: 227; SEQ ID NO: 215 and SEQ ID NO: 228; SEQ ID NO: 215 and SEQ ID NO: 229; SEQ ID NO: 215 and SEQ ID NO: 230; SEQ ID NO: 216 and SEQ ID NO: 217; SEQ ID NO: 216 and SEQ ID NO: 218; SEQ ID NO: 216 and SEQ ID NO: 219; SEQ ID NO: 216 and SEQ ID NO: 220; SEQ ID NO: 216 and SEQ ID NO: 221; SEQ ID NO: 216 and SEQ ID NO: 222; SEQ ID NO: 216 and SEQ ID NO: 223; SEQ ID NO: 216 and SEQ ID NO: 224; SEQ ID NO: 216 and SEQ ID NO: 225; SEQ ID NO: 216 and SEQ ID NO: 226; SEQ ID NO: 216 and SEQ ID NO: 227; SEQ ID NO: 216 and SEQ ID NO: 228; SEQ ID NO: 216 and SEQ ID NO: 229; SEQ ID NO: 216 and SEQ ID NO: 230; SEQ ID NO: 217 and SEQ ID NO: 218; SEQ ID NO: 217 and SEQ ID NO: 219; SEQ ID NO: 217 and SEQ ID NO: 220; SEQ ID NO: 217 and SEQ ID NO: 221; SEQ ID NO: 217 and SEQ ID NO: 222; SEQ ID NO: 217 and SEQ ID NO: 223; SEQ ID NO: 217 and SEQ ID NO: 224; SEQ ID NO: 217 and SEQ ID NO: 225; SEQ ID NO: 217 and SEQ ID NO: 226; SEQ ID NO: 217 and SEQ ID NO: 227; SEQ ID NO: 217 and SEQ ID NO: 228; SEQ ID NO: 217 and SEQ ID NO: 229; SEQ ID NO: 217 and SEQ ID NO: 230; SEQ ID NO: 218 and SEQ ID NO: 219; SEQ ID NO: 218 and SEQ ID NO: 220; SEQ ID NO: 218 and SEQ ID NO: 221; SEQ ID NO: 218 and SEQ ID NO: 222; SEQ ID NO: 218 and SEQ ID NO: 223; SEQ ID NO: 218 and SEQ ID NO: 224; SEQ ID NO: 218 and SEQ ID NO: 225; SEQ ID NO: 218 and SEQ ID NO: 226; SEQ ID NO: 218 and SEQ ID NO: 227; SEQ ID NO: 218 and SEQ ID NO: 228; SEQ ID NO: 218 and SEQ ID NO: 229; SEQ ID NO: 218 and SEQ ID NO: 230; SEQ ID NO: 219 and SEQ ID NO: 220; SEQ ID NO: 219 and SEQ ID NO: 221; SEQ ID NO: 219 and SEQ ID NO: 222; SEQ ID NO: 219 and SEQ ID NO: 223; SEQ ID NO: 219 and SEQ ID NO: 224; SEQ ID NO: 219 and SEQ ID NO: 225; SEQ ID NO: 219 and SEQ ID NO: 226; SEQ ID NO: 219 and SEQ ID NO: 227; SEQ ID NO: 219 and SEQ ID NO: 228; SEQ ID NO: 219 and SEQ ID NO: 229; SEQ ID NO: 219 and SEQ ID NO: 230; SEQ ID NO: 220 and SEQ ID NO: 221; SEQ ID NO: 220 and SEQ ID NO: 222; SEQ ID NO: 220 and SEQ ID NO: 223; SEQ ID NO: 220 and SEQ ID NO: 224; SEQ ID NO: 220 and SEQ ID NO: 225; SEQ ID NO: 220 and SEQ ID NO: 226; SEQ ID NO: 220 and SEQ ID NO: 227; SEQ ID NO: 220 and SEQ ID NO: 228; SEQ ID NO: 220 and SEQ ID NO: 229; SEQ ID NO: 220 and SEQ ID NO: 230; SEQ ID NO: 221 and SEQ ID NO: 222; SEQ ID NO: 221 and SEQ ID NO: 223; SEQ ID NO: 221 and SEQ ID NO: 224; SEQ ID NO: 221 and SEQ ID NO: 225; SEQ ID NO: 221 and SEQ ID NO: 226; SEQ ID NO: 221 and SEQ ID NO: 227; SEQ ID NO: 221 and SEQ ID NO: 228; SEQ ID NO: 221 and SEQ ID NO: 229; SEQ ID NO: 221 and SEQ ID NO: 230; SEQ ID NO: 222 and SEQ ID NO: 223; SEQ ID NO: 222 and SEQ ID NO: 224; SEQ ID NO: 222 and SEQ ID NO: 225; SEQ ID NO: 222 and SEQ ID NO: 226; SEQ ID NO: 222 and SEQ ID NO: 227; SEQ ID NO: 222 and SEQ ID NO: 228; SEQ ID NO: 222 and SEQ ID NO: 229; SEQ ID NO: 222 and SEQ ID NO: 230; SEQ ID NO: 223 and SEQ ID NO: 224; SEQ ID NO: 223 and SEQ ID NO: 225; SEQ ID NO: 223 and SEQ ID NO: 226; SEQ ID NO: 223 and SEQ ID NO: 227; SEQ ID NO: 223 and SEQ ID NO: 228; SEQ ID NO: 223 and SEQ ID NO: 229; SEQ ID NO: 223 and SEQ ID NO: 230; SEQ ID NO: 224 and SEQ ID NO: 225; SEQ ID NO: 224 and SEQ ID NO: 226; SEQ ID NO: 224 and SEQ ID NO: 227; SEQ ID NO: 224 and SEQ ID NO: 228; SEQ ID NO: 224 and SEQ ID NO: 229; SEQ ID NO: 224 and SEQ ID NO: 230; SEQ ID NO: 225 and SEQ ID NO: 226; SEQ ID NO: 225 and SEQ ID NO: 227; SEQ ID NO: 225 and SEQ ID NO: 228; SEQ ID NO: 225 and SEQ ID NO: 229; SEQ ID NO: 225 and SEQ ID NO: 230; SEQ ID NO: 226 and SEQ ID NO: 227; SEQ ID NO: 226 and SEQ ID NO: 228; SEQ ID NO: 226 and SEQ ID NO: 229; SEQ ID NO: 226 and SEQ ID NO: 230; SEQ ID NO: 227 and SEQ ID NO: 228; SEQ ID NO: 227 and SEQ ID NO: 229; SEQ ID NO: 227 and SEQ ID NO: 230; SEQ ID NO: 228 and SEQ ID NO: 229; SEQ ID NO: 228 and SEQ ID NO: 230; SEQ ID NO: 229 and SEQ ID NO: 230; SEQ ID NO: 231 and SEQ ID NO: 232; SEQ ID NO: 231 and SEQ ID NO: 233; SEQ ID NO: 231 and SEQ ID NO: 234; SEQ ID NO: 231 and SEQ ID NO: 235; SEQ ID NO: 231 and SEQ ID NO: 236; SEQ ID NO: 231 and SEQ ID NO: 237; SEQ ID NO: 231 and SEQ ID NO: 238; SEQ ID NO: 231 and SEQ ID NO: 239; SEQ ID NO: 231 and SEQ ID NO: 240; SEQ ID NO: 231 and SEQ ID NO: 241; SEQ ID NO: 231 and SEQ ID NO: 242; SEQ ID NO: 232 and SEQ ID NO: 233; SEQ ID NO: 232 and SEQ ID NO: 234; SEQ ID NO: 232 and SEQ ID NO: 235; SEQ ID NO: 232 and SEQ ID NO: 236; SEQ ID NO: 232 and SEQ ID NO: 237; SEQ ID NO: 232 and SEQ ID NO: 238; SEQ ID NO: 232 and SEQ ID NO: 239; SEQ ID NO: 232 and SEQ ID NO: 240; SEQ ID NO: 232 and SEQ ID NO: 241; SEQ ID NO: 232 and SEQ ID NO: 242; SEQ ID NO: 233 and SEQ ID NO: 234; SEQ ID NO: 233 and SEQ ID NO: 235; SEQ ID NO: 233 and SEQ ID NO: 236; SEQ ID NO: 233 and SEQ ID NO: 237; SEQ ID NO: 233 and SEQ ID NO: 238; SEQ ID NO: 233 and SEQ ID NO: 239; SEQ ID NO: 233 and SEQ ID NO: 240; SEQ ID NO: 233 and SEQ ID NO: 241; SEQ ID NO: 233 and SEQ ID NO: 242; SEQ ID NO: 234 and SEQ ID NO: 235; SEQ ID NO: 234 and SEQ ID NO: 236; SEQ ID NO: 234 and SEQ ID NO: 237; SEQ ID NO: 234 and SEQ ID NO: 238; SEQ ID NO: 234 and SEQ ID NO: 239; SEQ ID NO: 234 and SEQ ID NO: 240; SEQ ID NO: 234 and SEQ ID NO: 241; SEQ ID NO: 234 and SEQ ID NO: 242; SEQ ID NO: 235 and SEQ ID NO: 236; SEQ ID NO: 235 and SEQ ID NO: 237; SEQ ID NO: 235 and SEQ ID NO: 238; SEQ ID NO: 235 and SEQ ID NO: 239; SEQ ID NO: 235 and SEQ ID NO: 240; SEQ ID NO: 235 and SEQ ID NO: 241; SEQ ID NO: 235 and SEQ ID NO: 242; SEQ ID NO: 236 and SEQ ID NO: 237; SEQ ID NO: 236 and SEQ ID NO: 238; SEQ ID NO: 236 and SEQ ID NO: 239; SEQ ID NO: 236 and SEQ ID NO: 240; SEQ ID NO: 236 and SEQ ID NO: 241; SEQ ID NO: 236 and SEQ ID NO: 242; SEQ ID NO: 237 and SEQ ID NO: 238; SEQ ID NO: 237 and SEQ ID NO: 239; SEQ ID NO: 237 and SEQ ID NO: 240; SEQ ID NO: 237 and SEQ ID NO: 241; SEQ ID NO: 237 and SEQ ID NO: 242; SEQ ID NO: 238 and SEQ ID NO: 239; SEQ ID NO: 238 and SEQ ID NO: 240; SEQ ID NO: 238 and SEQ ID NO: 241; SEQ ID NO: 238 and SEQ ID NO: 242; SEQ ID NO: 239 and SEQ ID NO: 240; SEQ ID NO: 239 and SEQ ID NO: 241; SEQ ID NO: 239 and SEQ ID NO: 242; SEQ ID NO: 240 and SEQ ID NO: 241; SEQ ID NO: 240 and SEQ ID NO: 242; SEQ ID NO: 241 and SEQ ID NO: 242; SEQ ID NO: 243 and SEQ ID NO: 244; SEQ ID NO: 243 and SEQ ID NO: 245; SEQ ID NO: 243 and SEQ ID NO: 246; SEQ ID NO: 243 and SEQ ID NO: 247; SEQ ID NO: 243 and SEQ ID NO: 248; SEQ ID NO: 243 and SEQ ID NO: 249; SEQ ID NO: 243 and SEQ ID NO: 250; SEQ ID NO: 243 and SEQ ID NO: 251; SEQ ID NO: 243 and SEQ ID NO: 252; SEQ ID NO: 243 and SEQ ID NO: 253; SEQ ID NO: 243 and SEQ ID NO: 254; SEQ ID NO: 243 and SEQ ID NO: 255; SEQ ID NO: 243 and SEQ ID NO: 256; SEQ ID NO: 243 and SEQ ID NO: 257; SEQ ID NO: 243 and SEQ ID NO: 258; SEQ ID NO: 243 and SEQ ID NO: 259; SEQ ID NO: 243 and SEQ ID NO: 260; SEQ ID NO: 243 and SEQ ID NO: 261; SEQ ID NO: 243 and SEQ ID NO: 262; SEQ ID NO: 243 and SEQ ID NO: 263; SEQ ID NO: 243 and SEQ ID NO: 264; SEQ ID NO: 243 and SEQ ID NO: 265; SEQ ID NO: 243 and SEQ ID NO: 266; SEQ ID NO: 243 and SEQ ID NO: 267; SEQ ID NO: 243 and SEQ ID NO: 268; SEQ ID NO: 243 and SEQ ID NO: 269; SEQ ID NO: 244 and SEQ ID NO: 245; SEQ ID NO: 244 and SEQ ID NO: 246; SEQ ID NO: 244 and SEQ ID NO: 247; SEQ ID NO: 244 and SEQ ID NO: 248; SEQ ID NO: 244 and SEQ ID NO: 249; SEQ ID NO: 244 and SEQ ID NO: 250; SEQ ID NO: 244 and SEQ ID NO: 251; SEQ ID NO: 244 and SEQ ID NO: 252; SEQ ID NO: 244 and SEQ ID NO: 253; SEQ ID NO: 244 and SEQ ID NO: 254; SEQ ID NO: 244 and SEQ ID NO: 255; SEQ ID NO: 244 and SEQ ID NO: 256; SEQ ID NO: 244 and SEQ ID NO: 257; SEQ ID NO: 244 and SEQ ID NO: 258; SEQ ID NO: 244 and SEQ ID NO: 259; SEQ ID NO: 244 and SEQ ID NO: 260; SEQ ID NO: 244 and SEQ ID NO: 261; SEQ ID NO: 244 and SEQ ID NO: 262; SEQ ID NO: 244 and SEQ ID NO: 263; SEQ ID NO: 244 and SEQ ID NO: 264; SEQ ID NO: 244 and SEQ ID NO: 265; SEQ ID NO: 244 and SEQ ID NO: 266; SEQ ID NO: 244 and SEQ ID NO: 267; SEQ ID NO: 244 and SEQ ID NO: 268; SEQ ID NO: 244 and SEQ ID NO: 269; SEQ ID NO: 245 and SEQ ID NO: 246; SEQ ID NO: 245 and SEQ ID NO: 247; SEQ ID NO: 245 and SEQ ID NO: 248; SEQ ID NO: 245 and SEQ ID NO: 249; SEQ ID NO: 245 and SEQ ID NO: 250; SEQ ID NO: 245 and SEQ ID NO: 251; SEQ ID NO: 245 and SEQ ID NO: 252; SEQ ID NO: 245 and SEQ ID NO: 253; SEQ ID NO: 245 and SEQ ID NO: 254; SEQ ID NO: 245 and SEQ ID NO: 255; SEQ ID NO: 245 and SEQ ID NO: 256; SEQ ID NO: 245 and SEQ ID NO: 257; SEQ ID NO: 245 and SEQ ID NO: 258; SEQ ID NO: 245 and SEQ ID NO: 259; SEQ ID NO: 245 and SEQ ID NO: 260; SEQ ID NO: 245 and SEQ ID NO: 261; SEQ ID NO: 245 and SEQ ID NO: 262; SEQ ID NO: 245 and SEQ ID NO: 263; SEQ ID NO: 245 and SEQ ID NO: 264; SEQ ID NO: 245 and SEQ ID NO: 265; SEQ ID NO: 245 and SEQ ID NO: 266; SEQ ID NO: 245 and SEQ ID NO: 267; SEQ ID NO: 245 and SEQ ID NO: 268; SEQ ID NO: 245 and SEQ ID NO: 269; SEQ ID NO: 246 and SEQ ID NO: 247; SEQ ID NO: 246 and SEQ ID NO: 248; SEQ ID NO: 246 and SEQ ID NO: 249; SEQ ID NO: 246 and SEQ ID NO: 250; SEQ ID NO: 246 and SEQ ID NO: 251; SEQ ID NO: 246 and SEQ ID NO: 252; SEQ ID NO: 246 and SEQ ID NO: 253; SEQ ID NO: 246 and SEQ ID NO: 254; SEQ ID NO: 246 and SEQ ID NO: 255; SEQ ID NO: 246 and SEQ ID NO: 256; SEQ ID NO: 246 and SEQ ID NO: 257; SEQ ID NO: 246 and SEQ ID NO: 258; SEQ ID NO: 246 and SEQ ID NO: 259; SEQ ID NO: 246 and SEQ ID NO: 260; SEQ ID NO: 246 and SEQ ID NO: 261; SEQ ID NO: 246 and SEQ ID NO: 262; SEQ ID NO: 246 and SEQ ID NO: 263; SEQ ID NO: 246 and SEQ ID NO: 264; SEQ ID NO: 246 and SEQ ID NO: 265; SEQ ID NO: 246 and SEQ ID NO: 266; SEQ ID NO: 246 and SEQ ID NO: 267; SEQ ID NO: 246 and SEQ ID NO: 268; SEQ ID NO: 246 and SEQ ID NO: 269; SEQ ID NO: 247 and SEQ ID NO: 248; SEQ ID NO: 247 and SEQ ID NO: 249; SEQ ID NO: 247 and SEQ ID NO: 250; SEQ ID NO: 247 and SEQ ID NO: 251; SEQ ID NO: 247 and SEQ ID NO: 252; SEQ ID NO: 247 and SEQ ID NO: 253; SEQ ID NO: 247 and SEQ ID NO: 254; SEQ ID NO: 247 and SEQ ID NO: 255; SEQ ID NO: 247 and SEQ ID NO: 256; SEQ ID NO: 247 and SEQ ID NO: 257; SEQ ID NO: 247 and SEQ ID NO: 258; SEQ ID NO: 247 and SEQ ID NO: 259; SEQ ID NO: 247 and SEQ ID NO: 260; SEQ ID NO: 247 and SEQ ID NO: 261; SEQ ID NO: 247 and SEQ ID NO: 262; SEQ ID NO: 247 and SEQ ID NO: 263; SEQ ID NO: 247 and SEQ ID NO: 264; SEQ ID NO: 247 and SEQ ID NO: 265; SEQ ID NO: 247 and SEQ ID NO: 266; SEQ ID NO: 247 and SEQ ID NO: 267; SEQ ID NO: 247 and SEQ ID NO: 268; SEQ ID NO: 247 and SEQ ID NO: 269; SEQ ID NO: 248 and SEQ ID NO: 249; SEQ ID NO: 248 and SEQ ID NO: 250; SEQ ID NO: 248 and SEQ ID NO: 251; SEQ ID NO: 248 and SEQ ID NO: 252; SEQ ID NO: 248 and SEQ ID NO: 253; SEQ ID NO: 248 and SEQ ID NO: 254; SEQ ID NO: 248 and SEQ ID NO: 255; SEQ ID NO: 248 and SEQ ID NO: 256; SEQ ID NO: 248 and SEQ ID NO: 257; SEQ ID NO: 248 and SEQ ID NO: 258; SEQ ID NO: 248 and SEQ ID NO: 259; SEQ ID NO: 248 and SEQ ID NO: 260; SEQ ID NO: 248 and SEQ ID NO: 261; SEQ ID NO: 248 and SEQ ID NO: 262; SEQ ID NO: 248 and SEQ ID NO: 263; SEQ ID NO: 248 and SEQ ID NO: 264; SEQ ID NO: 248 and SEQ ID NO: 265; SEQ ID NO: 248 and SEQ ID NO: 266; SEQ ID NO: 248 and SEQ ID NO: 267; SEQ ID NO: 248 and SEQ ID NO: 268; SEQ ID NO: 248 and SEQ ID NO: 269; SEQ ID NO: 249 and SEQ ID NO: 250; SEQ ID NO: 249 and SEQ ID NO: 251; SEQ ID NO: 249 and SEQ ID NO: 252; SEQ ID NO: 249 and SEQ ID NO: 253; SEQ ID NO: 249 and SEQ ID NO: 254; SEQ ID NO: 249 and SEQ ID NO: 255; SEQ ID NO: 249 and SEQ ID NO: 256; SEQ ID NO: 249 and SEQ ID NO: 257; SEQ ID NO: 249 and SEQ ID NO: 258; SEQ ID NO: 249 and SEQ ID NO: 259; SEQ ID NO: 249 and SEQ ID NO: 260; SEQ ID NO: 249 and SEQ ID NO: 261; SEQ ID NO: 249 and SEQ ID NO: 262; SEQ ID NO: 249 and SEQ ID NO: 263; SEQ ID NO: 249 and SEQ ID NO: 264; SEQ ID NO: 249 and SEQ ID NO: 265; SEQ ID NO: 249 and SEQ ID NO: 266; SEQ ID NO: 249 and SEQ ID NO: 267; SEQ ID NO: 249 and SEQ ID NO: 268; SEQ ID NO: 249 and SEQ ID NO: 269; SEQ ID NO: 250 and SEQ ID NO: 251; SEQ ID NO: 250 and SEQ ID NO: 252; SEQ ID NO: 250 and SEQ ID NO: 253; SEQ ID NO: 250 and SEQ ID NO: 254; SEQ ID NO: 250 and SEQ ID NO: 255; SEQ ID NO: 250 and SEQ ID NO: 256; SEQ ID NO: 250 and SEQ ID NO: 257; SEQ ID NO: 250 and SEQ ID NO: 258; SEQ ID NO: 250 and SEQ ID NO: 259; SEQ ID NO: 250 and SEQ ID NO: 260; SEQ ID NO: 250 and SEQ ID NO: 261; SEQ ID NO: 250 and SEQ ID NO: 262; SEQ ID NO: 250 and SEQ ID NO: 263; SEQ ID NO: 250 and SEQ ID NO: 264; SEQ ID NO: 250 and SEQ ID NO: 265; SEQ ID NO: 250 and SEQ ID NO: 266; SEQ ID NO: 250 and SEQ ID NO: 267; SEQ ID NO: 250 and SEQ ID NO: 268; SEQ ID NO: 250 and SEQ ID NO: 269; SEQ ID NO: 251 and SEQ ID NO: 252; SEQ ID NO: 251 and SEQ ID NO: 253; SEQ ID NO: 251 and SEQ ID NO: 254; SEQ ID NO: 251 and SEQ ID NO: 255; SEQ ID NO: 251 and SEQ ID NO: 256; SEQ ID NO: 251 and SEQ ID NO: 257; SEQ ID NO: 251 and SEQ ID NO: 258; SEQ ID NO: 251 and SEQ ID NO: 259; SEQ ID NO: 251 and SEQ ID NO: 260; SEQ ID NO: 251 and SEQ ID NO: 261; SEQ ID NO: 251 and SEQ ID NO: 262; SEQ ID NO: 251 and SEQ ID NO: 263; SEQ ID NO: 251 and SEQ ID NO: 264; SEQ ID NO: 251 and SEQ ID NO: 265; SEQ ID NO: 251 and SEQ ID NO: 266; SEQ ID NO: 251 and SEQ ID NO: 267; SEQ ID NO: 251 and SEQ ID NO: 268; SEQ ID NO: 251 and SEQ ID NO: 269; SEQ ID NO: 252 and SEQ ID NO: 253; SEQ ID NO: 252 and SEQ ID NO: 254; SEQ ID NO: 252 and SEQ ID NO: 255; SEQ ID NO: 252 and SEQ ID NO: 256; SEQ ID NO: 252 and SEQ ID NO: 257; SEQ ID NO: 252 and SEQ ID NO: 258; SEQ ID NO: 252 and SEQ ID NO: 259; SEQ ID NO: 252 and SEQ ID NO: 260; SEQ ID NO: 252 and SEQ ID NO: 261; SEQ ID NO: 252 and SEQ ID NO: 262; SEQ ID NO: 252 and SEQ ID NO: 263; SEQ ID NO: 252 and SEQ ID NO: 264; SEQ ID NO: 252 and SEQ ID NO: 265; SEQ ID NO: 252 and SEQ ID NO: 266; SEQ ID NO: 252 and SEQ ID NO: 267; SEQ ID NO: 252 and SEQ ID NO: 268; SEQ ID NO: 252 and SEQ ID NO: 269; SEQ ID NO: 253 and SEQ ID NO: 254; SEQ ID NO: 253 and SEQ ID NO: 255; SEQ ID NO: 253 and SEQ ID NO: 256; SEQ ID NO: 253 and SEQ ID NO: 257; SEQ ID NO: 253 and SEQ ID NO: 258; SEQ ID NO: 253 and SEQ ID NO: 259; SEQ ID NO: 253 and SEQ ID NO: 260; SEQ ID NO: 253 and SEQ ID NO: 261; SEQ ID NO: 253 and SEQ ID NO: 262; SEQ ID NO: 253 and SEQ ID NO: 263; SEQ ID NO: 253 and SEQ ID NO: 264; SEQ ID NO: 253 and SEQ ID NO: 265; SEQ ID NO: 253 and SEQ ID NO: 266; SEQ ID NO: 253 and SEQ ID NO: 267; SEQ ID NO: 253 and SEQ ID NO: 268; SEQ ID NO: 253 and SEQ ID NO: 269; SEQ ID NO: 254 and SEQ ID NO: 255; SEQ ID NO: 254 and SEQ ID NO: 256; SEQ ID NO: 254 and SEQ ID NO: 257; SEQ ID NO: 254 and SEQ ID NO: 258; SEQ ID NO: 254 and SEQ ID NO: 259; SEQ ID NO: 254 and SEQ ID NO: 260; SEQ ID NO: 254 and SEQ ID NO: 261; SEQ ID NO: 254 and SEQ ID NO: 262; SEQ ID NO: 254 and SEQ ID NO: 263; SEQ ID NO: 254 and SEQ ID NO: 264; SEQ ID NO: 254 and SEQ ID NO: 265; SEQ ID NO: 254 and SEQ ID NO: 266; SEQ ID NO: 254 and SEQ ID NO: 267; SEQ ID NO: 254 and SEQ ID NO: 268; SEQ ID NO: 254 and SEQ ID NO: 269; SEQ ID NO: 255 and SEQ ID NO: 256; SEQ ID NO: 255 and SEQ ID NO: 257; SEQ ID NO: 255 and SEQ ID NO: 258; SEQ ID NO: 255 and SEQ ID NO: 259; SEQ ID NO: 255 and SEQ ID NO: 260; SEQ ID NO: 255 and SEQ ID NO: 261; SEQ ID NO: 255 and SEQ ID NO: 262; SEQ ID NO: 255 and SEQ ID NO: 263; SEQ ID NO: 255 and SEQ ID NO: 264; SEQ ID NO: 255 and SEQ ID NO: 265; SEQ ID NO: 255 and SEQ ID NO: 266; SEQ ID NO: 255 and SEQ ID NO: 267; SEQ ID NO: 255 and SEQ ID NO: 268; SEQ ID NO: 255 and SEQ ID NO: 269; SEQ ID NO: 256 and SEQ ID NO: 257; SEQ ID NO: 256 and SEQ ID NO: 258; SEQ ID NO: 256 and SEQ ID NO: 259; SEQ ID NO: 256 and SEQ ID NO: 260; SEQ ID NO: 256 and SEQ ID NO: 261; SEQ ID NO: 256 and SEQ ID NO: 262; SEQ ID NO: 256 and SEQ ID NO: 263; SEQ ID NO: 256 and SEQ ID NO: 264; SEQ ID NO: 256 and SEQ ID NO: 265; SEQ ID NO: 256 and SEQ ID NO: 266; SEQ ID NO: 256 and SEQ ID NO: 267; SEQ ID NO: 256 and SEQ ID NO: 268; SEQ ID NO: 256 and SEQ ID NO: 269; SEQ ID NO: 257 and SEQ ID NO: 258; SEQ ID NO: 257 and SEQ ID NO: 259; SEQ ID NO: 257 and SEQ ID NO: 260; SEQ ID NO: 257 and SEQ ID NO: 261; SEQ ID NO: 257 and SEQ ID NO: 262; SEQ ID NO: 257 and SEQ ID NO: 263; SEQ ID NO: 257 and SEQ ID NO: 264; SEQ ID NO: 257 and SEQ ID NO: 265; SEQ ID NO: 257 and SEQ ID NO: 266; SEQ ID NO: 257 and SEQ ID NO: 267; SEQ ID NO: 257 and SEQ ID NO: 268; SEQ ID NO: 257 and SEQ ID NO: 269; SEQ ID NO: 258 and SEQ ID NO: 259; SEQ ID NO: 258 and SEQ ID NO: 260; SEQ ID NO: 258 and SEQ ID NO: 261; SEQ ID NO: 258 and SEQ ID NO: 262; SEQ ID NO: 258 and SEQ ID NO: 263; SEQ ID NO: 258 and SEQ ID NO: 264; SEQ ID NO: 258 and SEQ ID NO: 265; SEQ ID NO: 258 and SEQ ID NO: 266; SEQ ID NO: 258 and SEQ ID NO: 267; SEQ ID NO: 258 and SEQ ID NO: 268; SEQ ID NO: 258 and SEQ ID NO: 269; SEQ ID NO: 259 and SEQ ID NO: 260; SEQ ID NO: 259 and SEQ ID NO: 261; SEQ ID NO: 259 and SEQ ID NO: 262; SEQ ID NO: 259 and SEQ ID NO: 263; SEQ ID NO: 259 and SEQ ID NO: 264; SEQ ID NO: 259 and SEQ ID NO: 265; SEQ ID NO: 259 and SEQ ID NO: 266; SEQ ID NO: 259 and SEQ ID NO: 267; SEQ ID NO: 259 and SEQ ID NO: 268; SEQ ID NO: 259 and SEQ ID NO: 269; SEQ ID NO: 260 and SEQ ID NO: 261; SEQ ID NO: 260 and SEQ ID NO: 262; SEQ ID NO: 260 and SEQ ID NO: 263; SEQ ID NO: 260 and SEQ ID NO: 264; SEQ ID NO: 260 and SEQ ID NO: 265; SEQ ID NO: 260 and SEQ ID NO: 266; SEQ ID NO: 260 and SEQ ID NO: 267; SEQ ID NO: 260 and SEQ ID NO: 268; SEQ ID NO: 260 and SEQ ID NO: 269; SEQ ID NO: 261 and SEQ ID NO: 262; SEQ ID NO: 261 and SEQ ID NO: 263; SEQ ID NO: 261 and SEQ ID NO: 264; SEQ ID NO: 261 and SEQ ID NO: 265; SEQ ID NO: 261 and SEQ ID NO: 266; SEQ ID NO: 261 and SEQ ID NO: 267; SEQ ID NO: 261 and SEQ ID NO: 268; SEQ ID NO: 261 and SEQ ID NO: 269; SEQ ID NO: 262 and SEQ ID NO: 263; SEQ ID NO: 262 and SEQ ID NO: 264; SEQ ID NO: 262 and SEQ ID NO: 265; SEQ ID NO: 262 and SEQ ID NO: 266; SEQ ID NO: 262 and SEQ ID NO: 267; SEQ ID NO: 262 and SEQ ID NO: 268; SEQ ID NO: 262 and SEQ ID NO: 269; SEQ ID NO: 263 and SEQ ID NO: 264; SEQ ID NO: 263 and SEQ ID NO: 265; SEQ ID NO: 263 and SEQ ID NO: 266; SEQ ID NO: 263 and SEQ ID NO: 267; SEQ ID NO: 263 and SEQ ID NO: 268; SEQ ID NO: 263 and SEQ ID NO: 269; SEQ ID NO: 264 and SEQ ID NO: 265; SEQ ID NO: 264 and SEQ ID NO: 266; SEQ ID NO: 264 and SEQ ID NO: 267; SEQ ID NO: 264 and SEQ ID NO: 268; SEQ ID NO: 264 and SEQ ID NO: 269; SEQ ID NO: 265 and SEQ ID NO: 266; SEQ ID NO: 265 and SEQ ID NO: 267; SEQ ID NO: 265 and SEQ ID NO: 268; SEQ ID NO: 265 and SEQ ID NO: 269; SEQ ID NO: 266 and SEQ ID NO: 267; SEQ ID NO: 266 and SEQ ID NO: 268; SEQ ID NO: 266 and SEQ ID NO: 269; SEQ ID NO: 267 and SEQ ID NO: 268; SEQ ID NO: 267 and SEQ ID NO: 269; SEQ ID NO: 268 and SEQ ID NO: 269; SEQ ID NO: 270 and SEQ ID NO: 271; SEQ ID NO: 270 and SEQ ID NO: 272; SEQ ID NO: 270 and SEQ ID NO: 273; SEQ ID NO: 270 and SEQ ID NO: 274; SEQ ID NO: 270 and SEQ ID NO: 275; SEQ ID NO: 270 and SEQ ID NO: 276; SEQ ID NO: 270 and SEQ ID NO: 277; SEQ ID NO: 270 and SEQ ID NO: 278; SEQ ID NO: 270 and SEQ ID NO: 279; SEQ ID NO: 270 and SEQ ID NO: 280; SEQ ID NO: 270 and SEQ ID NO: 281; SEQ ID NO: 270 and SEQ ID NO: 282; SEQ ID NO: 270 and SEQ ID NO: 283; SEQ ID NO: 270 and SEQ ID NO: 284; SEQ ID NO: 270 and SEQ ID NO: 285; SEQ ID NO: 270 and SEQ ID NO: 286; SEQ ID NO: 270 and SEQ ID NO: 287; SEQ ID NO: 270 and SEQ ID NO: 288; SEQ ID NO: 270 and SEQ ID NO: 289; SEQ ID NO: 270 and SEQ ID NO: 290; SEQ ID NO: 270 and SEQ ID NO: 291; SEQ ID NO: 270 and SEQ ID NO: 292; SEQ ID NO: 270 and SEQ ID NO: 924; SEQ ID NO: 270 and SEQ ID NO: 925; SEQ ID NO: 270 and SEQ ID NO: 926; SEQ ID NO: 270 and SEQ ID NO: 927; SEQ ID NO: 270 and SEQ ID NO: 928; SEQ ID NO: 270 and SEQ ID NO: 929; SEQ ID NO: 270 and SEQ ID NO: 930; SEQ ID NO: 270 and SEQ ID NO: 931; SEQ ID NO: 270 and SEQ ID NO: 932; SEQ ID NO: 270 and SEQ ID NO: 933; SEQ ID NO: 270 and SEQ ID NO: 934; SEQ ID NO: 270 and SEQ ID NO: 935; SEQ ID NO: 270 and SEQ ID NO: 936; SEQ ID NO: 270 and SEQ ID NO: 937; SEQ ID NO: 270 and SEQ ID NO: 938; SEQ ID NO: 271 and SEQ ID NO: 272; SEQ ID NO: 271 and SEQ ID NO: 273; SEQ ID NO: 271 and SEQ ID NO: 274; SEQ ID NO: 271 and SEQ ID NO: 275; SEQ ID NO: 271 and SEQ ID NO: 276; SEQ ID NO: 271 and SEQ ID NO: 277; SEQ ID NO: 271 and SEQ ID NO: 278; SEQ ID NO: 271 and SEQ ID NO: 279; SEQ ID NO: 271 and SEQ ID NO: 280; SEQ ID NO: 271 and SEQ ID NO: 281; SEQ ID NO: 271 and SEQ ID NO: 282; SEQ ID NO: 271 and SEQ ID NO: 283; SEQ ID NO: 271 and SEQ ID NO: 284; SEQ ID NO: 271 and SEQ ID NO: 285; SEQ ID NO: 271 and SEQ ID NO: 286; SEQ ID NO: 271 and SEQ ID NO: 287; SEQ ID NO: 271 and SEQ ID NO: 288; SEQ ID NO: 271 and SEQ ID NO: 289; SEQ ID NO: 271 and SEQ ID NO: 290; SEQ ID NO: 271 and SEQ ID NO: 291; SEQ ID NO: 271 and SEQ ID NO: 292; SEQ ID NO: 272 and SEQ ID NO: 924; SEQ ID NO: 272 and SEQ ID NO: 925; SEQ ID NO: 272 and SEQ ID NO: 926; SEQ ID NO: 272 and SEQ ID NO: 927; SEQ ID NO: 272 and SEQ ID NO: 928; SEQ ID NO: 272 and SEQ ID NO: 929; SEQ ID NO: 272 and SEQ ID NO: 930; SEQ ID NO: 272 and SEQ ID NO: 931; SEQ ID NO: 272 and SEQ ID NO: 932; SEQ ID NO: 272 and SEQ ID NO: 933; SEQ ID NO: 272 and SEQ ID NO: 934; SEQ ID NO: 272 and SEQ ID NO: 935; SEQ ID NO: 272 and SEQ ID NO: 936; SEQ ID NO: 272 and SEQ ID NO: 937; SEQ ID NO: 272 and SEQ ID NO: 938; SEQ ID NO: 273 and SEQ ID NO: 924; SEQ ID NO: 273 and SEQ ID NO: 925; SEQ ID NO: 273 and SEQ ID NO: 926; SEQ ID NO: 273 and SEQ ID NO: 927; SEQ ID NO: 273 and SEQ ID NO: 928; SEQ ID NO: 273 and SEQ ID NO: 929; SEQ ID NO: 273 and SEQ ID NO: 930; SEQ ID NO: 273 and SEQ ID NO: 931; SEQ ID NO: 273 and SEQ ID NO: 932; SEQ ID NO: 273 and SEQ ID NO: 933; SEQ ID NO: 273 and SEQ ID NO: 934; SEQ ID NO: 273 and SEQ ID NO: 935; SEQ ID NO: 273 and SEQ ID NO: 936; SEQ ID NO: 273 and SEQ ID NO: 937; SEQ ID NO: 273 and SEQ ID NO: 938; SEQ ID NO: 274 and SEQ ID NO: 924; SEQ ID NO: 274 and SEQ ID NO: 925; SEQ ID NO: 274 and SEQ ID NO: 926; SEQ ID NO: 274 and SEQ ID NO: 927; SEQ ID NO: 274 and SEQ ID NO: 928; SEQ ID NO: 274 and SEQ ID NO: 929; SEQ ID NO: 274 and SEQ ID NO: 930; SEQ ID NO: 274 and SEQ ID NO: 931; SEQ ID NO: 274 and SEQ ID NO: 932; SEQ ID NO: 274 and SEQ ID NO: 933; SEQ ID NO: 274 and SEQ ID NO: 934; SEQ ID NO: 274 and SEQ ID NO: 935; SEQ ID NO: 274 and SEQ ID NO: 936; SEQ ID NO: 274 and SEQ ID NO: 937; SEQ ID NO: 274 and SEQ ID NO: 938; SEQ ID NO: 275 and SEQ ID NO: 924; SEQ ID NO: 275 and SEQ ID NO: 925; SEQ ID NO: 275 and SEQ ID NO: 926; SEQ ID NO: 275 and SEQ ID NO: 927; SEQ ID NO: 275 and SEQ ID NO: 928; SEQ ID NO: 275 and SEQ ID NO: 929; SEQ ID NO: 275 and SEQ ID NO: 930; SEQ ID NO: 275 and SEQ ID NO: 931; SEQ ID NO: 275 and SEQ ID NO: 932; SEQ ID NO: 275 and SEQ ID NO: 933; SEQ ID NO: 275 and SEQ ID NO: 934; SEQ ID NO: 275 and SEQ ID NO: 935; SEQ ID NO: 275 and SEQ ID NO: 936; SEQ ID NO: 275 and SEQ ID NO: 937; SEQ ID NO: 275 and SEQ ID NO: 938; SEQ ID NO: 276 and SEQ ID NO: 924; SEQ ID NO: 276 and SEQ ID NO: 925; SEQ ID NO: 276 and SEQ ID NO: 926; SEQ ID NO: 276 and SEQ ID NO: 927; SEQ ID NO: 276 and SEQ ID NO: 928; SEQ ID NO: 276 and SEQ ID NO: 929; SEQ ID NO: 276 and SEQ ID NO: 930; SEQ ID NO: 276 and SEQ ID NO: 931; SEQ ID NO: 276 and SEQ ID NO: 932; SEQ ID NO: 276 and SEQ ID NO: 933; SEQ ID NO: 276 and SEQ ID NO: 934; SEQ ID NO: 276 and SEQ ID NO: 935; SEQ ID NO: 276 and SEQ ID NO: 936; SEQ ID NO: 276 and SEQ ID NO: 937; SEQ ID NO: 276 and SEQ ID NO: 938; SEQ ID NO: 277 and SEQ ID NO: 924; SEQ ID NO: 277 and SEQ ID NO: 925; SEQ ID NO: 277 and SEQ ID NO: 926; SEQ ID NO: 277 and SEQ ID NO: 927; SEQ ID NO: 277 and SEQ ID NO: 928; SEQ ID NO: 277 and SEQ ID NO: 929; SEQ ID NO: 277 and SEQ ID NO: 930; SEQ ID NO: 277 and SEQ ID NO: 931; SEQ ID NO: 277 and SEQ ID NO: 932; SEQ ID NO: 277 and SEQ ID NO: 933; SEQ ID NO: 277 and SEQ ID NO: 934; SEQ ID NO: 277 and SEQ ID NO: 935; SEQ ID NO: 277 and SEQ ID NO: 936; SEQ ID NO: 277 and SEQ ID NO: 937; SEQ ID NO: 277 and SEQ ID NO: 938; SEQ ID NO: 278 and SEQ ID NO: 924; SEQ ID NO: 278 and SEQ ID NO: 925; SEQ ID NO: 278 and SEQ ID NO: 926; SEQ ID NO: 278 and SEQ ID NO: 927; SEQ ID NO: 278 and SEQ ID NO: 928; SEQ ID NO: 278 and SEQ ID NO: 929; SEQ ID NO: 278 and SEQ ID NO: 930; SEQ ID NO: 278 and SEQ ID NO: 931; SEQ ID NO: 278 and SEQ ID NO: 932; SEQ ID NO: 278 and SEQ ID NO: 933; SEQ ID NO: 278 and SEQ ID NO: 934; SEQ ID NO: 278 and SEQ ID NO: 935; SEQ ID NO: 278 and SEQ ID NO: 936; SEQ ID NO: 278 and SEQ ID NO: 937; SEQ ID NO: 278 and SEQ ID NO: 938; SEQ ID NO: 279 and SEQ ID NO: 924; SEQ ID NO: 279 and SEQ ID NO: 925; SEQ ID NO: 279 and SEQ ID NO: 926; SEQ ID NO: 279 and SEQ ID NO: 927; SEQ ID NO: 279 and SEQ ID NO: 928; SEQ ID NO: 279 and SEQ ID NO: 929; SEQ ID NO: 279 and SEQ ID NO: 930; SEQ ID NO: 279 and SEQ ID NO: 931; SEQ ID NO: 279 and SEQ ID NO: 932; SEQ ID NO: 279 and SEQ ID NO: 933; SEQ ID NO: 279 and SEQ ID NO: 934; SEQ ID NO: 279 and SEQ ID NO: 935; SEQ ID NO: 279 and SEQ ID NO: 936; SEQ ID NO: 279 and SEQ ID NO: 937; SEQ ID NO: 279 and SEQ ID NO: 938; SEQ ID NO: 280 and SEQ ID NO: 924; SEQ ID NO: 280 and SEQ ID NO: 925; SEQ ID NO: 280 and SEQ ID NO: 926; SEQ ID NO: 280 and SEQ ID NO: 927; SEQ ID NO: 280 and SEQ ID NO: 928; SEQ ID NO: 280 and SEQ ID NO: 929; SEQ ID NO: 280 and SEQ ID NO: 930; SEQ ID NO: 280 and SEQ ID NO: 931; SEQ ID NO: 280 and SEQ ID NO: 932; SEQ ID NO: 280 and SEQ ID NO: 933; SEQ ID NO: 280 and SEQ ID NO: 934; SEQ ID NO: 280 and SEQ ID NO: 935; SEQ ID NO: 280 and SEQ ID NO: 936; SEQ ID NO: 280 and SEQ ID NO: 937; SEQ ID NO: 280 and SEQ ID NO: 938; SEQ ID NO: 281 and SEQ ID NO: 924; SEQ ID NO: 281 and SEQ ID NO: 925; SEQ ID NO: 281 and SEQ ID NO: 926; SEQ ID NO: 281 and SEQ ID NO: 927; SEQ ID NO: 281 and SEQ ID NO: 928; SEQ ID NO: 281 and SEQ ID NO: 929; SEQ ID NO: 281 and SEQ ID NO: 930; SEQ ID NO: 281 and SEQ ID NO: 931; SEQ ID NO: 281 and SEQ ID NO: 932; SEQ ID NO: 281 and SEQ ID NO: 933; SEQ ID NO: 281 and SEQ ID NO: 934; SEQ ID NO: 281 and SEQ ID NO: 935; SEQ ID NO: 281 and SEQ ID NO: 936; SEQ ID NO: 281 and SEQ ID NO: 937; SEQ ID NO: 281 and SEQ ID NO: 938; SEQ ID NO: 282 and SEQ ID NO: 924; SEQ ID NO: 282 and SEQ ID NO: 925; SEQ ID NO: 282 and SEQ ID NO: 926; SEQ ID NO: 282 and SEQ ID NO: 927; SEQ ID NO: 282 and SEQ ID NO: 928; SEQ ID NO: 282 and SEQ ID NO: 929; SEQ ID NO: 282 and SEQ ID NO: 930; SEQ ID NO: 282 and SEQ ID NO: 931; SEQ ID NO: 282 and SEQ ID NO: 932; SEQ ID NO: 282 and SEQ ID NO: 933; SEQ ID NO: 282 and SEQ ID NO: 934; SEQ ID NO: 282 and SEQ ID NO: 935; SEQ ID NO: 282 and SEQ ID NO: 936; SEQ ID NO: 282 and SEQ ID NO: 937; SEQ ID NO: 282 and SEQ ID NO: 938; SEQ ID NO: 283 and SEQ ID NO: 924; SEQ ID NO: 283 and SEQ ID NO: 925; SEQ ID NO: 283 and SEQ ID NO: 926; SEQ ID NO: 283 and SEQ ID NO: 927; SEQ ID NO: 283 and SEQ ID NO: 928; SEQ ID NO: 283 and SEQ ID NO: 929; SEQ ID NO: 283 and SEQ ID NO: 930; SEQ ID NO: 283 and SEQ ID NO: 931; SEQ ID NO: 283 and SEQ ID NO: 932; SEQ ID NO: 283 and SEQ ID NO: 933; SEQ ID NO: 283 and SEQ ID NO: 934; SEQ ID NO: 283 and SEQ ID NO: 935; SEQ ID NO: 283 and SEQ ID NO: 936; SEQ ID NO: 283 and SEQ ID NO: 937; SEQ ID NO: 283 and SEQ ID NO: 938; SEQ ID NO: 284 and SEQ ID NO: 924; SEQ ID NO: 284 and SEQ ID NO: 925; SEQ ID NO: 284 and SEQ ID NO: 926; SEQ ID NO: 284 and SEQ ID NO: 927; SEQ ID NO: 284 and SEQ ID NO: 928; SEQ ID NO: 284 and SEQ ID NO: 929; SEQ ID NO: 284 and SEQ ID NO: 930; SEQ ID NO: 284 and SEQ ID NO: 931; SEQ ID NO: 284 and SEQ ID NO: 932; SEQ ID NO: 284 and SEQ ID NO: 933; SEQ ID NO: 284 and SEQ ID NO: 934; SEQ ID NO: 284 and SEQ ID NO: 935; SEQ ID NO: 284 and SEQ ID NO: 936; SEQ ID NO: 284 and SEQ ID NO: 937; SEQ ID NO: 284 and SEQ ID NO: 938; SEQ ID NO: 285 and SEQ ID NO: 924; SEQ ID NO: 285 and SEQ ID NO: 925; SEQ ID NO: 285 and SEQ ID NO: 926; SEQ ID NO: 285 and SEQ ID NO: 927; SEQ ID NO: 285 and SEQ ID NO: 928; SEQ ID NO: 285 and SEQ ID NO: 929; SEQ ID NO: 285 and SEQ ID NO: 930; SEQ ID NO: 285 and SEQ ID NO: 931; SEQ ID NO: 285 and SEQ ID NO: 932; SEQ ID NO: 285 and SEQ ID NO: 933; SEQ ID NO: 285 and SEQ ID NO: 934; SEQ ID NO: 285 and SEQ ID NO: 935; SEQ ID NO: 285 and SEQ ID NO: 936; SEQ ID NO: 285 and SEQ ID NO: 937; SEQ ID NO: 285 and SEQ ID NO: 938; SEQ ID NO: 286 and SEQ ID NO: 924; SEQ ID NO: 286 and SEQ ID NO: 925; SEQ ID NO: 286 and SEQ ID NO: 926; SEQ ID NO: 286 and SEQ ID NO: 927; SEQ ID NO: 286 and SEQ ID NO: 928; SEQ ID NO: 286 and SEQ ID NO: 929; SEQ ID NO: 286 and SEQ ID NO: 930; SEQ ID NO: 286 and SEQ ID NO: 931; SEQ ID NO: 286 and SEQ ID NO: 932; SEQ ID NO: 286 and SEQ ID NO: 933; SEQ ID NO: 286 and SEQ ID NO: 934; SEQ ID NO: 286 and SEQ ID NO: 935; SEQ ID NO: 286 and SEQ ID NO: 936; SEQ ID NO: 286 and SEQ ID NO: 937; SEQ ID NO: 286 and SEQ ID NO: 938; SEQ ID NO: 287 and SEQ ID NO: 924; SEQ ID NO: 287 and SEQ ID NO: 925; SEQ ID NO: 287 and SEQ ID NO: 926; SEQ ID NO: 287 and SEQ ID NO: 927; SEQ ID NO: 287 and SEQ ID NO: 928; SEQ ID NO: 287 and SEQ ID NO: 929; SEQ ID NO: 287 and SEQ ID NO: 930; SEQ ID NO: 287 and SEQ ID NO: 931; SEQ ID NO: 287 and SEQ ID NO: 932; SEQ ID NO: 287 and SEQ ID NO: 933; SEQ ID NO: 287 and SEQ ID NO: 934; SEQ ID NO: 287 and SEQ ID NO: 935; SEQ ID NO: 287 and SEQ ID NO: 936; SEQ ID NO: 287 and SEQ ID NO: 937; SEQ ID NO: 287 and SEQ ID NO: 938; SEQ ID NO: 288 and SEQ ID NO: 924; SEQ ID NO: 288 and SEQ ID NO: 925; SEQ ID NO: 288 and SEQ ID NO: 926; SEQ ID NO: 288 and SEQ ID NO: 927; SEQ ID NO: 288 and SEQ ID NO: 928; SEQ ID NO: 288 and SEQ ID NO: 929; SEQ ID NO: 288 and SEQ ID NO: 930; SEQ ID NO: 288 and SEQ ID NO: 931; SEQ ID NO: 288 and SEQ ID NO: 932; SEQ ID NO: 288 and SEQ ID NO: 933; SEQ ID NO: 288 and SEQ ID NO: 934; SEQ ID NO: 288 and SEQ ID NO: 935; SEQ ID NO: 288 and SEQ ID NO: 936; SEQ ID NO: 288 and SEQ ID NO: 937; SEQ ID NO: 288 and SEQ ID NO: 938; SEQ ID NO: 289 and SEQ ID NO: 924; SEQ ID NO: 289 and SEQ ID NO: 925; SEQ ID NO: 289 and SEQ ID NO: 926; SEQ ID NO: 289 and SEQ ID NO: 927; SEQ ID NO: 289 and SEQ ID NO: 928; SEQ ID NO: 289 and SEQ ID NO: 929; SEQ ID NO: 289 and SEQ ID NO: 930; SEQ ID NO: 289 and SEQ ID NO: 931; SEQ ID NO: 289 and SEQ ID NO: 932; SEQ ID NO: 289 and SEQ ID NO: 933; SEQ ID NO: 289 and SEQ ID NO: 934; SEQ ID NO: 289 and SEQ ID NO: 935; SEQ ID NO: 289 and SEQ ID NO: 936; SEQ ID NO: 289 and SEQ ID NO: 937; SEQ ID NO: 289 and SEQ ID NO: 938; SEQ ID NO: 290 and SEQ ID NO: 924; SEQ ID NO: 290 and SEQ ID NO: 925; SEQ ID NO: 290 and SEQ ID NO: 926; SEQ ID NO: 290 and SEQ ID NO: 927; SEQ ID NO: 290 and SEQ ID NO: 928; SEQ ID NO: 290 and SEQ ID NO: 929; SEQ ID NO: 290 and SEQ ID NO: 930; SEQ ID NO: 290 and SEQ ID NO: 931; SEQ ID NO: 290 and SEQ ID NO: 932; SEQ ID NO: 290 and SEQ ID NO: 933; SEQ ID NO: 290 and SEQ ID NO: 934; SEQ ID NO: 290 and SEQ ID NO: 935; SEQ ID NO: 290 and SEQ ID NO: 936; SEQ ID NO: 290 and SEQ ID NO: 937; SEQ ID NO: 290 and SEQ ID NO: 938; SEQ ID NO: 291 and SEQ ID NO: 924; SEQ ID NO: 291 and SEQ ID NO: 925; SEQ ID NO: 291 and SEQ ID NO: 926; SEQ ID NO: 291 and SEQ ID NO: 927; SEQ ID NO: 291 and SEQ ID NO: 928; SEQ ID NO: 291 and SEQ ID NO: 929; SEQ ID NO: 291 and SEQ ID NO: 930; SEQ ID NO: 291 and SEQ ID NO: 931; SEQ ID NO: 291 and SEQ ID NO: 932; SEQ ID NO: 291 and SEQ ID NO: 933; SEQ ID NO: 291 and SEQ ID NO: 934; SEQ ID NO: 291 and SEQ ID NO: 935; SEQ ID NO: 291 and SEQ ID NO: 936; SEQ ID NO: 291 and SEQ ID NO: 937; SEQ ID NO: 291 and SEQ ID NO: 938; SEQ ID NO: 292 and SEQ ID NO: 924; SEQ ID NO: 292 and SEQ ID NO: 925; SEQ ID NO: 292 and SEQ ID NO: 926; SEQ ID NO: 292 and SEQ ID NO: 927; SEQ ID NO: 292 and SEQ ID NO: 928; SEQ ID NO: 292 and SEQ ID NO: 929; SEQ ID NO: 292 and SEQ ID NO: 930; SEQ ID NO: 292 and SEQ ID NO: 931; SEQ ID NO: 292 and SEQ ID NO: 932; SEQ ID NO: 292 and SEQ ID NO: 933; SEQ ID NO: 292 and SEQ ID NO: 934; SEQ ID NO: 292 and SEQ ID NO: 935; SEQ ID NO: 292 and SEQ ID NO: 936; SEQ ID NO: 292 and SEQ ID NO: 937; SEQ ID NO: 292 and SEQ ID NO: 938; SEQ ID NO: 272 and SEQ ID NO: 273; SEQ ID NO: 272 and SEQ ID NO: 274; SEQ ID NO: 272 and SEQ ID NO: 275; SEQ ID NO: 272 and SEQ ID NO: 276; SEQ ID NO: 272 and SEQ ID NO: 277; SEQ ID NO: 272 and SEQ ID NO: 278; SEQ ID NO: 272 and SEQ ID NO: 279; SEQ ID NO: 272 and SEQ ID NO: 280; SEQ ID NO: 272 and SEQ ID NO: 281; SEQ ID NO: 272 and SEQ ID NO: 282; SEQ ID NO: 272 and SEQ ID NO: 283; SEQ ID NO: 272 and SEQ ID NO: 284; SEQ ID NO: 272 and SEQ ID NO: 285; SEQ ID NO: 272 and SEQ ID NO: 286; SEQ ID NO: 272 and SEQ ID NO: 287; SEQ ID NO: 272 and SEQ ID NO: 288; SEQ ID NO: 272 and SEQ ID NO: 289; SEQ ID NO: 272 and SEQ ID NO: 290; SEQ ID NO: 272 and SEQ ID NO: 291; SEQ ID NO: 272 and SEQ ID NO: 292; SEQ ID NO: 273 and SEQ ID NO: 274; SEQ ID NO: 273 and SEQ ID NO: 275; SEQ ID NO: 273 and SEQ ID NO: 276; SEQ ID NO: 273 and SEQ ID NO: 277; SEQ ID NO: 273 and SEQ ID NO: 278; SEQ ID NO: 273 and SEQ ID NO: 279; SEQ ID NO: 273 and SEQ ID NO: 280; SEQ ID NO: 273 and SEQ ID NO: 281; SEQ ID NO: 273 and SEQ ID NO: 282; SEQ ID NO: 273 and SEQ ID NO: 283; SEQ ID NO: 273 and SEQ ID NO: 284; SEQ ID NO: 273 and SEQ ID NO: 285; SEQ ID NO: 273 and SEQ ID NO: 286; SEQ ID NO: 273 and SEQ ID NO: 287; SEQ ID NO: 273 and SEQ ID NO: 288; SEQ ID NO: 273 and SEQ ID NO: 289; SEQ ID NO: 273 and SEQ ID NO: 290; SEQ ID NO: 273 and SEQ ID NO: 291; SEQ ID NO: 273 and SEQ ID NO: 292; SEQ ID NO: 274 and SEQ ID NO: 275; SEQ ID NO: 274 and SEQ ID NO: 276; SEQ ID NO: 274 and SEQ ID NO: 277; SEQ ID NO: 274 and SEQ ID NO: 278; SEQ ID NO: 274 and SEQ ID NO: 279; SEQ ID NO: 274 and SEQ ID NO: 280; SEQ ID NO: 274 and SEQ ID NO: 281; SEQ ID NO: 274 and SEQ ID NO: 282; SEQ ID NO: 274 and SEQ ID NO: 283; SEQ ID NO: 274 and SEQ ID NO: 284; SEQ ID NO: 274 and SEQ ID NO: 285; SEQ ID NO: 274 and SEQ ID NO: 286; SEQ ID NO: 274 and SEQ ID NO: 287; SEQ ID NO: 274 and SEQ ID NO: 288; SEQ ID NO: 274 and SEQ ID NO: 289; SEQ ID NO: 274 and SEQ ID NO: 290; SEQ ID NO: 274 and SEQ ID NO: 291; SEQ ID NO: 274 and SEQ ID NO: 292; SEQ ID NO: 275 and SEQ ID NO: 276; SEQ ID NO: 275 and SEQ ID NO: 277; SEQ ID NO: 275 and SEQ ID NO: 278; SEQ ID NO: 275 and SEQ ID NO: 279; SEQ ID NO: 275 and SEQ ID NO: 280; SEQ ID NO: 275 and SEQ ID NO: 281; SEQ ID NO: 275 and SEQ ID NO: 282; SEQ ID NO: 275 and SEQ ID NO: 283; SEQ ID NO: 275 and SEQ ID NO: 284; SEQ ID NO: 275 and SEQ ID NO: 285; SEQ ID NO: 275 and SEQ ID NO: 286; SEQ ID NO: 275 and SEQ ID NO: 287; SEQ ID NO: 275 and SEQ ID NO: 288; SEQ ID NO: 275 and SEQ ID NO: 289; SEQ ID NO: 275 and SEQ ID NO: 290; SEQ ID NO: 275 and SEQ ID NO: 291; SEQ ID NO: 275 and SEQ ID NO: 292; SEQ ID NO: 276 and SEQ ID NO: 277; SEQ ID NO: 276 and SEQ ID NO: 278; SEQ ID NO: 276 and SEQ ID NO: 279; SEQ ID NO: 276 and SEQ ID NO: 280; SEQ ID NO: 276 and SEQ ID NO: 281; SEQ ID NO: 276 and SEQ ID NO: 282; SEQ ID NO: 276 and SEQ ID NO: 283; SEQ ID NO: 276 and SEQ ID NO: 284; SEQ ID NO: 276 and SEQ ID NO: 285; SEQ ID NO: 276 and SEQ ID NO: 286; SEQ ID NO: 276 and SEQ ID NO: 287; SEQ ID NO: 276 and SEQ ID NO: 288; SEQ ID NO: 276 and SEQ ID NO: 289; SEQ ID NO: 276 and SEQ ID NO: 290; SEQ ID NO: 276 and SEQ ID NO: 291; SEQ ID NO: 276 and SEQ ID NO: 292; SEQ ID NO: 277 and SEQ ID NO: 278; SEQ ID NO: 277 and SEQ ID NO: 279; SEQ ID NO: 277 and SEQ ID NO: 280; SEQ ID NO: 277 and SEQ ID NO: 281; SEQ ID NO: 277 and SEQ ID NO: 282; SEQ ID NO: 277 and SEQ ID NO: 283; SEQ ID NO: 277 and SEQ ID NO: 284; SEQ ID NO: 277 and SEQ ID NO: 285; SEQ ID NO: 277 and SEQ ID NO: 286; SEQ ID NO: 277 and SEQ ID NO: 287; SEQ ID NO: 277 and SEQ ID NO: 288; SEQ ID NO: 277 and SEQ ID NO: 289; SEQ ID NO: 277 and SEQ ID NO: 290; SEQ ID NO: 277 and SEQ ID NO: 291; SEQ ID NO: 277 and SEQ ID NO: 292; SEQ ID NO: 278 and SEQ ID NO: 279; SEQ ID NO: 278 and SEQ ID NO: 280; SEQ ID NO: 278 and SEQ ID NO: 281; SEQ ID NO: 278 and SEQ ID NO: 282; SEQ ID NO: 278 and SEQ ID NO: 283; SEQ ID NO: 278 and SEQ ID NO: 284; SEQ ID NO: 278 and SEQ ID NO: 285; SEQ ID NO: 278 and SEQ ID NO: 286; SEQ ID NO: 278 and SEQ ID NO: 287; SEQ ID NO: 278 and SEQ ID NO: 288; SEQ ID NO: 278 and SEQ ID NO: 289; SEQ ID NO: 278 and SEQ ID NO: 290; SEQ ID NO: 278 and SEQ ID NO: 291; SEQ ID NO: 278 and SEQ ID NO: 292; SEQ ID NO: 279 and SEQ ID NO: 280; SEQ ID NO: 279 and SEQ ID NO: 281; SEQ ID NO: 279 and SEQ ID NO: 282; SEQ ID NO: 279 and SEQ ID NO: 283; SEQ ID NO: 279 and SEQ ID NO: 284; SEQ ID NO: 279 and SEQ ID NO: 285; SEQ ID NO: 279 and SEQ ID NO: 286; SEQ ID NO: 279 and SEQ ID NO: 287; SEQ ID NO: 279 and SEQ ID NO: 288; SEQ ID NO: 279 and SEQ ID NO: 289; SEQ ID NO: 279 and SEQ ID NO: 290; SEQ ID NO: 279 and SEQ ID NO: 291; SEQ ID NO: 279 and SEQ ID NO: 292; SEQ ID NO: 280 and SEQ ID NO: 281; SEQ ID NO: 280 and SEQ ID NO: 282; SEQ ID NO: 280 and SEQ ID NO: 283; SEQ ID NO: 280 and SEQ ID NO: 284; SEQ ID NO: 280 and SEQ ID NO: 285; SEQ ID NO: 280 and SEQ ID NO: 286; SEQ ID NO: 280 and SEQ ID NO: 287; SEQ ID NO: 280 and SEQ ID NO: 288; SEQ ID NO: 280 and SEQ ID NO: 289; SEQ ID NO: 280 and SEQ ID NO: 290; SEQ ID NO: 280 and SEQ ID NO: 291; SEQ ID NO: 280 and SEQ ID NO: 292; SEQ ID NO: 281 and SEQ ID NO: 282; SEQ ID NO: 281 and SEQ ID NO: 283; SEQ ID NO: 281 and SEQ ID NO: 284; SEQ ID NO: 281 and SEQ ID NO: 285; SEQ ID NO: 281 and SEQ ID NO: 286; SEQ ID NO: 281 and SEQ ID NO: 287; SEQ ID NO: 281 and SEQ ID NO: 288; SEQ ID NO: 281 and SEQ ID NO: 289; SEQ ID NO: 281 and SEQ ID NO: 290; SEQ ID NO: 281 and SEQ ID NO: 291; SEQ ID NO: 281 and SEQ ID NO: 292; SEQ ID NO: 282 and SEQ ID NO: 283; SEQ ID NO: 282 and SEQ ID NO: 284; SEQ ID NO: 282 and SEQ ID NO: 285; SEQ ID NO: 282 and SEQ ID NO: 286; SEQ ID NO: 282 and SEQ ID NO: 287; SEQ ID NO: 282 and SEQ ID NO: 288; SEQ ID NO: 282 and SEQ ID NO: 289; SEQ ID NO: 282 and SEQ ID NO: 290; SEQ ID NO: 282 and SEQ ID NO: 291; SEQ ID NO: 282 and SEQ ID NO: 292; SEQ ID NO: 283 and SEQ ID NO: 284; SEQ ID NO: 283 and SEQ ID NO: 285; SEQ ID NO: 283 and SEQ ID NO: 286; SEQ ID NO: 283 and SEQ ID NO: 287; SEQ ID NO: 283 and SEQ ID NO: 288; SEQ ID NO: 283 and SEQ ID NO: 289; SEQ ID NO: 283 and SEQ ID NO: 290; SEQ ID NO: 283 and SEQ ID NO: 291; SEQ ID NO: 283 and SEQ ID NO: 292; SEQ ID NO: 284 and SEQ ID NO: 285; SEQ ID NO: 284 and SEQ ID NO: 286; SEQ ID NO: 284 and SEQ ID NO: 287; SEQ ID NO: 284 and SEQ ID NO: 288; SEQ ID NO: 284 and SEQ ID NO: 289; SEQ ID NO: 284 and SEQ ID NO: 290; SEQ ID NO: 284 and SEQ ID NO: 291; SEQ ID NO: 284 and SEQ ID NO: 292; SEQ ID NO: 285 and SEQ ID NO: 286; SEQ ID NO: 285 and SEQ ID NO: 287; SEQ ID NO: 285 and SEQ ID NO: 288; SEQ ID NO: 285 and SEQ ID NO: 289; SEQ ID NO: 285 and SEQ ID NO: 290; SEQ ID NO: 285 and SEQ ID NO: 291; SEQ ID NO: 285 and SEQ ID NO: 292; SEQ ID NO: 286 and SEQ ID NO: 287; SEQ ID NO: 286 and SEQ ID NO: 288; SEQ ID NO: 286 and SEQ ID NO: 289; SEQ ID NO: 286 and SEQ ID NO: 290; SEQ ID NO: 286 and SEQ ID NO: 291; SEQ ID NO: 286 and SEQ ID NO: 292; SEQ ID NO: 287 and SEQ ID NO: 288; SEQ ID NO: 287 and SEQ ID NO: 289; SEQ ID NO: 287 and SEQ ID NO: 290; SEQ ID NO: 287 and SEQ ID NO: 291; SEQ ID NO: 287 and SEQ ID NO: 292; SEQ ID NO: 288 and SEQ ID NO: 289; SEQ ID NO: 288 and SEQ ID NO: 290; SEQ ID NO: 288 and SEQ ID NO: 291; SEQ ID NO: 288 and SEQ ID NO: 292; SEQ ID NO: 289 and SEQ ID NO: 290; SEQ ID NO: 289 and SEQ ID NO: 291; SEQ ID NO: 289 and SEQ ID NO: 292; SEQ ID NO: 290 and SEQ ID NO: 291; SEQ ID NO: 290 and SEQ ID NO: 292; SEQ ID NO: 291 and SEQ ID NO: 292.

In one aspect, a composition is provided comprising a single nucleic acid molecule encoding, or two nucleic acid molecules where one molecule encodes, 1) one or more guide RNA that comprises a guide sequence selected from any one of SEQ ID NOs: 1-159; and 2) a SaCas9. In one aspect, a composition is provided comprising a single nucleic acid molecule encoding, or two nucleic acid molecules where one molecule encodes, 1) one or more guide RNA that comprises a guide sequence selected from any one of SEQ ID NOs: 200-292, 924-938, or 950-955; and 2) a SluCas9.

In one aspect, a composition is provided comprising a single nucleic acid molecule encoding, or two nucleic acid molecules where one molecule encodes, a) one or more guide RNA that comprises a guide sequence that is at least 99%, 98%, 97%, 96%, 95%, 94%, 93%, 92%, 91%, or 90% identical to any one of SEQ ID NOs: 1-159 and a SaCas9; or b) one or more guide RNA that comprises a guide sequence that is at least 99%, 98%, 97%, 96%, 95%, 94%, 93%, 92%, 91%, or 90% identical to any one of SEQ ID NOs: 200-292, 924-938, or 950-955; and a SluCas9. In one aspect, a composition is provided comprising a single nucleic acid molecule encoding a) one or more guide RNA that comprises a guide sequence that is at least 99%, 98%, 97%, 96%, 95%, 94%, 93%, 92%, 91%, or 90% identical to any one of 1) SEQ ID NOs: 200-292, 924-938, or 950-955, and a SluCas9; or b) one or more guide RNA that comprises a guide sequence that is at least 99%, 98%, 97%, 96%, 95%, 94%, 93%, 92%, 91%, or 90% identical to any one of SEQ ID NOs: 1-159, and a SaCas9.

In another aspect, a composition is provided comprising a single nucleic acid molecule encoding, or two nucleic acid molecules where one molecule encodes, a) one or more guide RNA that comprises a guide sequence comprising at least 16, 17, 18, 19, or 20 contiguous nucleotides of a spacer sequence selected from any one of SEQ ID NOs: 1-159 and a SaCas9; or b) one or more guide RNA that comprises a guide sequence comprising at least 16, 17, 18, 19, or 20 contiguous nucleotides of a spacer sequence selected from any one of SEQ ID NOs: 200-292, 924-938, or 950-955 and a SluCas9. In another aspect, a composition is provided comprising a single nucleic acid molecule encoding, or two nucleic acid molecules where one molecule encodes, a) one or more guide RNA that comprises a guide sequence comprising at least 16, 17, 18, 19, or 20 contiguous nucleotides of a spacer sequence selected from any one of SEQ ID NOs: 1-159 and a SaCas9; or b) one or more guide RNA that comprises a guide sequence comprising at least 16, 17, 18, 19, or 20 contiguous nucleotides of a spacer sequence selected from any one of SEQ ID NOs: 200-292, 924-938, or 950-955 and a SluCas9.

In some embodiments, any of the guides disclosed herein may be used as a research tool, e.g., to study trafficking, expression, and processing by cells.

### Scaffold Sequences

Each of the guide sequences shown in Table 6 may further comprise additional nucleotides to form or encode a crRNA, e.g., using any known sequence appropriate for the Cas9 being used. In some embodiments, the crRNA comprises (5′ to 3′) at least a spacer sequence and a first complementarity domain. The first complementary domain is sufficiently complementary to a second complementarity domain, which may be part ofthe same molecule in the case of an sgRNA or in a tracrRNA in the case of a dual or modular gRNA, to form a duplex. See, e.g., US 2017/0007679 for detailed discussion of crRNA and gRNA domains, including first and second complementarity domains.

A single-molecule guide RNA (sgRNA) can comprise, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and/or an optional tracrRNA extension sequence. The optional tracrRNA extension can comprise elements that contribute additional functionality (e.g., stability) to the guide RNA. The single-molecule guide linker can link the minimum CRISPR repeat and the minimum tracrRNA sequence to form a hairpin structure. The optional tracrRNA extension can comprise one or more hairpins. In particular embodiments, the disclosure provides for an sgRNA comprising a spacer sequence and a tracrRNA sequence.

The guide RNA can be considered to comprise a scaffold sequence necessary for endonuclease binding and a spacer sequence required to bind to the genomic target sequence. In some embodiments, the guide RNA comprises any of the scaffold sequences disclosed herein and any of the spacer sequences disclosed herein. In some embodiments, the guide RNA comprises any of the scaffold sequences disclosed herein and any of the spacer sequences disclosed herein, without any nucleotides between the scaffold sequence and the spacer sequence.

An exemplary scaffold sequence suitable for use with SaCas9 to follow the guide sequence at its 3′ end is: GTTTAAGTACTCTGTGCTGGAAACAGCACAGAATCTACTTAAACAAGGCAAAATGCCGT GTTTATCTCGTCAACTTGTTGGCGAGA (SEQ ID NO: 500) in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 500, or a sequence that differs from SEQ ID NO: 500 by no more than 1, 2, 3, 4, 5, 10, 15, 20, or 25 nucleotides.

In some embodiments, a variant of an SaCas9 scaffold sequence may be used. In some embodiments, the SaCas9 scaffold to follow the guide sequence at its 3′ end is referred to as “SaScaffoldV1” and is: GTTTTAGTACTCTGGAAACAGAATCTACTAAAACAAGGCAAAATGCCGTGTTTATCTCGT CAACTTGTTGGCGAGAT (SEQ ID NO: 501) in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 501, or a sequence that differs from SEQ ID NO: 501 by no more than 1, 2, 3, 4, 5, 10, 15, 20, or 25 nucleotides.

In some embodiments, a variant of an SaCas9 scaffold sequence may be used. In some embodiments, the SaCas9 scaffold to follow the guide sequence at its 3′ end is referred to as “SaScaffoldV2” and is: GTTTAAGTACTCTGTGCTGGAAACAGCACAGAATCTACTTAAACAAGGCAAAATGCCGT GTTTATCTCGTCAACTTGTTGGCGAGAT (SEQ ID NO: 502) in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 502, or a sequence that differs from SEQ ID NO: 502 by no more than 1, 2, 3, 4, 5, 10, 15, 20, or 25 nucleotides.

In some embodiments, a variant of an SaCas9 scaffold sequence may be used. In some embodiments, the SaCas9 scaffold to follow the guide sequence at its 3′ end is referred to as “SaScaffoldV3” and is: GTTTAAGTACTCTGGAAACAGAATCTACTTAAACAAGGCAAAATGCCGTGTTTATCTCGT CAACTTGTTGGCGAGAT (SEQ ID NO: 503) in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 503, or a sequence that differs from SEQ ID NO: 503 by no more than 1, 2, 3, 4, 5, 10, 15, 20, or 25 nucleotides.

In some embodiments, a variant of an SaCas9 scaffold sequence may be used. In some embodiments, the SaCas9 scaffold to follow the guide sequence at its 3′ end is referred to as “SaScaffoldV5” and is: GTTTCAGTACTCTGGAAACAGAATCTACTGAAACAAGGCAAAATGCCGTGTTTATCTCGT CAACTTGTTGGCGAGAT (SEQ ID NO: 504) in 5′ to 3′ orientation. In some embodiments, an exemplary scaffold sequence for use with SaCas9 to follow the 3′ end ofthe guide sequence is a sequence that is at least 80%, 85% o, 90%, 91%, 92% o, 93%, 94%, 95%, 96% o, 97%, 98% o, 99% o or 10000 identical to SEQ ID NO: 504, or a sequence that differs from SEQ ID NO: 504 by no more than 1, 2, 3, 4, 5, 10, 15, 20, or 25 nucleotides.

Two exemplary scaffold sequences suitable for use with SluCas9 to follow the guide sequence at its 3′ end are: GTTTTAGTACTCTGGAAACAGAATCTACTGAAACAAGACAATATGTCGTGTTTATCCCAT CAATTTATTGGTGGGA (SEQ ID NO: 600), or GTTTAAGTACTCTGTGCTGGAAACAGCACAGAATCTACTGAAACAAGACAATATGTCGT GTTTATCCCATCAATTTATTGGTGGGA (SEQ ID NO: 601) in 5′ to 3′ orientation. In some embodiments, an exemplary sequence for use with SluCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 600 or SEQ ID NO: 601, or a sequence that differs from SEQ ID NO: 600 or SEQ ID NO: 601 by no more than 1, 2, 3, 4, 5, 10, 15, 20, or 25 nucleotides.

Exemplary scaffold sequences suitable for use with SluCas9 to follow the guide sequence at its 3′ end are also shown below in the 5′ to 3′ orientation.

TABLE 7

| | | | | Streak of |
|---|---|---|---|---|
| | | | | Homology to |
| Scaffold | SEQ | | Homology | Slu v5 |
| ID | ID NO | Scaffold Sequence (5′ to 3′) | to Slu v5 | (# nucleotides) |
| Wildtype | 900 | GTTTTAGTACTCTGGAAACAGAATCTACTGAA | N/A | N/A |
| | | ACAAGACAATATGTCGTGTTTATCCCATCAAT | | |
| | | TTATTGGTGGGAT | | |
| Slu- | 601 | GTTTAAGTACTCTGTGCTGGAAACAGCACAG | N/A | N/A |
| VCGT- | | AATCTACTGAAACAAGACAATATGTCGTGTTT | | |
| 4.5 | | ATCCCATCAATTTATTGGTGGGA | | |
| Slu_v5 | 901 | GTTTCAGTACTCTGGAAACAGAATCTACTGAA | 100.00% | 77 |
| | | ACAAGACAATATGTCGTGTTTATCCCATCAAT | | |
| | | TTATTGGTGGGAT | | |
| Slu_v5-1 | 902 | GTTTggTaACcTaGGAAACTagATCTTaccAAACA | 87.50% | 47 |
| | | AGACAATATGTCGTGTTTATCCCATCAATTTA | | |
| | | TTGGTGGGAT | | |
| Slu_v5-2 | 903 | GTTTCAGTACTCTGGAAACAGAATCTACTGAA | 96.10% | 37 |
| | | ACAAGgCAAaATGcCGTGTTTATCCCATCAATT | | |
| | | TATTGGTGGGAT | | |
| Slu_v5-3 | 904 | GTTTCAGTACTCTGGAAACAGAATCTACTGAA | 94.81% | 48 |
| | | ACAAGACAATATGTCGcgcccaTCCCATCAATTT | | |
| | | ATTGGTGGGAT | | |
| Slu_v5-4 | 905 | GTTTCAGTACTCTGGAAACAGAATCTACTGAA | 91.55% | 55 |
| | | ACAAGACAATATGTCGTGTTTATgggTTgAATT | | |
| | | TATTcGacccAT | | |
| Slu_v5-5 | 906 | GTTTggTaACcTaGGAAACTagATCTTaccAAACA | 83.75% | 31 |
| | | AGgCAAaATGcCGTGTTTATCCCATCAATTTAT | | |
| | | TGGTGGGAT | | |
| Slu_v5-6 | 907 | GTTTggTaACcTaGGAAACTagATCTTaccAAACA | 82.50% | 23 |
| | | AGACAATATGTCGcgcccaTCCCATCAATTTATT | | |
| | | GGTGGGAT | | |
| Slu_v5-7 | 908 | GTTTggTaACcTaGGAAACTagATCTTaccAAACA | 78.38% | 25 |
| | | AGACAATATGTCGTGTTTATgggTTgAATTTAT | | |
| | | TcGacccAT | | |
| Slu_v5-8 | 909 | GTTTCAGTACTCTGGAAACAGAATCTACTGAA | 90.91% | 37 |
| | | ACAAGgCAAaATGcCGcgcccaTCCCATCAATTTA | | |
| | | TTGGTGGGAT | | |
| Slu_v5-9 | 910 | GTTTCAGTACTCTGGAAACAGAATCTACTGAA | 87.32% | 37 |
| | | ACAAGgCAAaATGcCGTGTTTATgggTTgAATTT | | |
| | | ATTcGacccAT | | |
| Slu_v5- | 911 | GTTTCAGTACTCTGGAAACAGAATCTACTGAA | 82.89% | 48 |
| 10 | | ACAAGACAATATGTCGcgcccaTgggTTgAATTTA | | |
| | | TTcGacccAT | | |
| Slu_v5- | 912 | GTTTggTaACcTaGGAAACTagATCTTaccAAACA | 78.75% | 23 |
| 11 | | AGgCAAaATGcCGcgcccaTCCCATCAATTTATTG | | |
| | | GTGGGAT | | |
| Slu_v5- | 913 | GTTTggTaACcTaGGAAACTagATCTTaccAAACA | 74.32% | 9 |
| 12 | | AGgCAAaATGcCGTGTTTATgggTTgAATTTATTc | | |
| | | GacccAT | | |
| Slu_v5- | 914 | GTTTggTaACcTaGGAAACTagATCTTaccAAACA | 70.89% | 18 |
| 13 | | AGACAATATGTCGcgcccaTgggTTgAATTTATTc | | |
| | | GacccAT | | |
| Slu_v5- | 915 | GTTTCAGTACTCTGGAAACAGAATCTACTGAA | 78.95% | 37 |
| 14 | | ACAAGgCAAaATGcCGcgcccaTgggTTgAATTTAT | | |
| | | TcGacccAT | | |
| Slu_v5- | 916 | GTTTggTaACcTaGGAAACTagATCTTaccAAACA | 67.09% | 8 |
| 15 | | AGgCAAaATGcCGcgcccaTgggTTgAATTTATTcGa | | |
| | | cccAT | | |
| Slu_v4 | 917 | GTTTCAGTACTCTGTGCTGGAAACAGCACAGA | N/A | N/A |
| | | ATCTACTGAAACAAGACAATATGTCGTGTTTA | | |
| | | TCCCATCAATTTATTGGTGGGAT | | |

In some embodiments, the scaffold sequence suitable for use with SaCas9 to follow the guide sequence at its 3′ end is selected from any one of SEQ ID NOs: 500-504 in 5′ to 3 orientation. In some embodiments, an exemplary sequence for use with SaCas9 to follow the 3′ end of the guide sequence is a sequence that is at least 65% o, 70%, 75%, 80%, 85% o, 90%, 91%, 92% o, 93%, 94%, 95%, 96% o, 97%, 98% o, 99% o or 100% o identical to any one off SEQ ID NOs: 500-504, or a sequence that differs from any one of SEQ ID NOs: 500-504 by no more than 1, 2, 3, 4, 5, 10, 15, 20, or 25 nucleotides.

In some embodiments, the scaffold sequence suitable for use with SluCas9 to follow the guide sequence at its 3′ end is selected from any one of SEQ ID NOs: 900 or 601, or 901-917 in 5′ to 3 orientation. In some embodiments, an exemplary sequence for use with SluCas9 to follow the 3′ end of the guide sequence is a sequence that is at least 65% o, 70%, 75%, 80%, 85% o, 90% o, 91% o, 92% o, 93%, 94%, 95% o, 96% o, 97%, 98% o, 99% o or 100% o identical to any one off SEQ ID NOs: 900 or 601, or 901-917, or a sequence that differs from any one of SEQ ID NOs: 900 or 601, or 901-917 by no more than 1, 2, 3, 4, 5, 10, 15, 20, or 25 nucleotides.

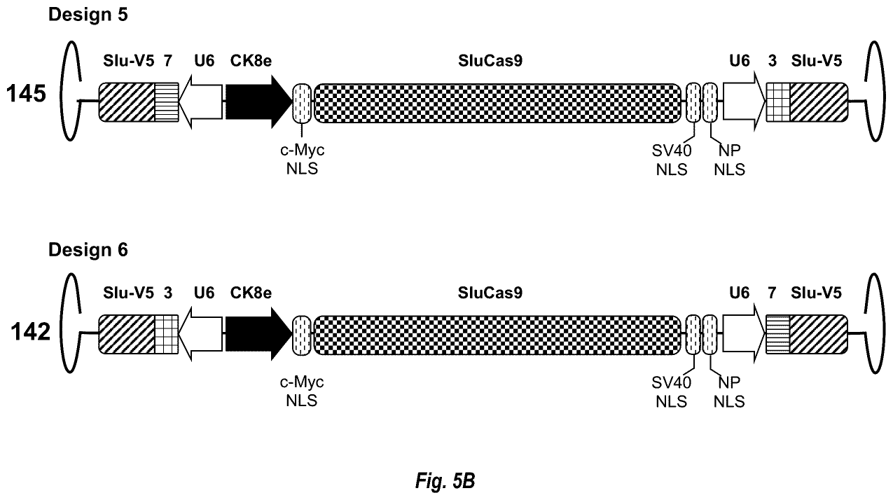

In some embodiments, any scaffold sequence (e.g., any of the scaffold sequences disclosed herein) is suitable for use with any sRGN disclosed herein, such as any one of sRGN1, sRGN2, sRGN3, sRGN3.1, sRGN3.2, sRGN3.3, sRGN4, or a nucleic acid molecule encoding the same. In some embodiments, a scaffold sequence suitable for use with a sRGN, such as at the 3′ end of a guide RNA sequence within an expression vector comprising the sRGN (e.g., an expression vector as shown in FIGS. 5A-5B) is selected from any one of SEQ ID NOs: 500-504, 601, or 900-917. In some embodiments, a scaffold sequence suitable for use with a sRGN, such as at the 3′ end of a guide RNA sequence within an expression vector comprising the sRGN (e.g., an expression vector as shown in FIGS. 5A-5B) is a sequence that is at least 65%, 70%, 75%, 80%, 85%, 90%, 91%, 92%, 93%, 94%, 95%, 96%, 97%, 98%, 99% or 100% identical to any one of SEQ ID NOs: 500-504, 601, or 900-917, or a sequence that differs from any one of SEQ ID NOs: 500-504, 601, or 900-917 by no more than 1, 2, 3, 4, 5, 10, 15, 20, or 25 nucleotides.

In some embodiments, the nucleic acid encoding the gRNA comprises a sequence comprising SEQ ID NO: 500. In some embodiments, the nucleic acid encoding the gRNA comprises a sequence comprising SEQ ID NO: 501. In some embodiments, the nucleic acid encoding the gRNA comprises a sequence comprising SEQ ID NO: 502. In some embodiments, the nucleic acid encoding the gRNA comprises a sequence comprising SEQ ID NO: 503. In some embodiments, the nucleic acid encoding the gRNA comprises a sequence comprising SEQ ID NO: 504. In some embodiments, comprising a pair of gRNAs, one of the gRNAs comprises a sequence selected from any one of SEQ ID NOs: 500-504. In some embodiments, comprising a pair of gRNAs, both of the gRNAs comprise a sequence selected from any one of SEQ ID NOs: 500-504. In some embodiments, comprising a pair of gRNAs, the nucleotides 3′ of the guide sequence of the gRNAs are the same sequence. In some embodiments, comprising a pair of gRNAs, the nucleotides 3′ of the guide sequence of the gRNAs are different sequences.

In some embodiments, the nucleic acid encoding the gRNA comprises a sequence comprising SEQ ID NO: 900. In some embodiments, the nucleic acid encoding the gRNA comprises a sequence comprising SEQ ID NO: 601. In some embodiments, the nucleic acid encoding the gRNA comprises a sequence comprising SEQ ID NO: 900. In some embodiments, the nucleic acid encoding the gRNA comprises a sequence comprising SEQ ID NO: 901. In some embodiments, the nucleic acid encoding the gRNA comprises a sequence comprising SEQ ID NO: 902. In some embodiments, the nucleic acid encoding the gRNA comprises a sequence comprising SEQ ID NO: 903. In some embodiments, the nucleic acid encoding the gRNA comprises a sequence comprising SEQ ID NO: 904. In some embodiments, the nucleic acid encoding the gRNA comprises a sequence comprising SEQ ID NO: 905. In some embodiments, the nucleic acid encoding the gRNA comprises a sequence comprising SEQ ID NO: 906. In some embodiments, the nucleic acid encoding the gRNA comprises a sequence comprising SEQ ID NO: 907. In some embodiments, the nucleic acid encoding the gRNA comprises a sequence comprising SEQ ID NO: 908. In some embodiments, the nucleic acid encoding the gRNA comprises a sequence comprising SEQ ID NO: 909. In some embodiments, the nucleic acid encoding the gRNA comprises a sequence comprising SEQ ID NO: 910. In some embodiments, the nucleic acid encoding the gRNA comprises a sequence comprising SEQ ID NO: 911. In some embodiments, the nucleic acid encoding the gRNA comprises a sequence comprising SEQ ID NO: 912. In some embodiments, the nucleic acid encoding the gRNA comprises a sequence comprising SEQ ID NO: 913. In some embodiments, the nucleic acid encoding the gRNA comprises a sequence comprising SEQ ID NO: 914. In some embodiments, the nucleic acid encoding the gRNA comprises a sequence comprising SEQ ID NO: 915. In some embodiments, the nucleic acid encoding the gRNA comprises a sequence comprising SEQ ID NO: 916. In some embodiments, the nucleic acid encoding the gRNA comprises a sequence comprising SEQ ID NO: 917. In some embodiments, comprising a pair of gRNAs, one of the gRNAs comprises a sequence selected from any one of SEQ ID NOs: 900 or 601, or 901-917. In some embodiments, comprising a pair of gRNAs, both of the gRNAs comprise a sequence selected from any one of SEQ ID NOs: 900 or 601, or 901-917. In some embodiments, comprising a pair of gRNAs, the nucleotides 3′ of the guide sequence of the gRNAs are the same sequence. In some embodiments, comprising a pair of gRNAs, the nucleotides 3′ of the guide sequence of the gRNAs are different sequences.

In some embodiments, the scaffold sequence comprises one or more alterations in the stem loop 1 as compared to the stem loop 1 of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). In some embodiments, the scaffold sequence comprises one or more alterations in the stem loop 2 as compared to the stem loop 2 of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). In some embodiments, the scaffold sequence comprises one or more alterations in the tetraloop as compared to the tetraloop of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). In some embodiments, the scaffold sequence comprises one or more alterations in the repeat region as compared to the repeat region of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). In some embodiments, the scaffold sequence comprises one or more alterations in the anti-repeat region as compared to the anti-repeat region of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). In some embodiments, the scaffold sequence comprises one or more alterations in the linker region as compared to the linker region of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). See, e.g., Nishimasu et al., 2015, Cell, 162:1113-1126 for description of regions of a scaffold.

Where a tracrRNA is used, in some embodiments, it comprises (5′ to 3′) a second complementary domain and a proximal domain. In the case of a sgRNA, guide sequences together with additional nucleotides (e.g., SEQ ID Nos: 500-504 (for SaCas9), and 900 or 601, or 901-917 (for SluCas9)) form or encode a sgRNA. In some embodiments, an sgRNA comprises (5′ to 3′) at least a spacer sequence, a first complementary domain, a linking domain, a second complementary domain, and a proximal domain. A sgRNA or tracrRNA may further comprise a tail domain. The linking domain may be hairpin-forming. See, e.g., US 2017/0007679 for detailed discussion and examples of crRNA and gRNA domains, including second complementarity domains, linking domains, proximal domains, and tail domains.

In some embodiments, the disclosure provides for specific nucleic acid sequences encoding one or more guide RNA components (e.g., any of the spacer and or scaffold sequences disclosed herein). The disclosure contemplates RNA equivalents of any of the DNA sequences provided herein (i.e., in which “T”s are replaced with “U”s), or DNA equivalents of any of the RNA sequences provided herein (i.e., in which “U”s are replaced with “T”s), as well as complements (including reverse complements) of any of the sequences disclosed herein.

In some embodiments, a composition is provided comprising a guide RNA, or nucleic acid encoding a guide RNA, wherein the guide RNA further comprises a trRNA. In each composition and method embodiment described herein, the crRNA (comprising the spacer sequence) and trRNA may be associated as a single RNA (sgRNA) or may be on separate RNAs (dgRNA). In the context of sgRNAs, the crRNA and trRNA components may be covalently linked, e.g., via a phosphodiester bond or other covalent bond.

### Vectors

In any embodiment comprising a nucleic acid molecule encoding a guide RNA and/or a Cas9, the nucleic acid molecule may be a vector. In some embodiments, a composition is provided comprising a single nucleic acid molecule encoding at least one guide RNA and Cas9, wherein the nucleic acid molecule is a vector. In some embodiments, a composition is provided comprising more than one nucleic acid molecule encoding a guide RNA and Cas9, wherein the nucleic acid molecule is a vector. In some embodiments, a composition is provided comprising more than one nucleic acid molecule wherein one molecule encodes one or more guide RNA, and the other molecule encodes Cas9 plus or minus at least one guide RNA, wherein the nucleic acid molecule is a vector.

Any type of vector, such as any of those described herein, may be used. In some embodiments, the vector is a lipid nanoparticle. In some embodiments, the vector is a viral vector. In some embodiments, the viral vector is a non-integrating viral vector (i.e., that does not insert sequence from the vector into a host chromosome). In some embodiments, the viral vector is an adeno-associated virus vector (AAV), a lentiviral vector, an integrase-deficient lentiviral vector, an adenoviral vector, a vaccinia viral vector, an alphaviral vector, or a herpes simplex viral vector. In some embodiments, the vector comprises a muscle-specific promoter. Exemplary muscle-specific promoters include a muscle creatine kinase promoter, a desmin promoter, an MHCK7 promoter, or an SPc5-12 promoter. See US 2004/0175727 A1; Wang et al., Expert Opin Drug Deliv. (2014) 11, 345-364; Wang et al., Gene Therapy (2008) 15, 1489-1499. In some embodiments, the muscle-specific promoter is a CK8 promoter. In some embodiments, the muscle-specific promoter is a CK8e promoter. In any of the foregoing embodiments, the vector may be an adeno-associated virus vector (AAV).

In some embodiments, the vector is a lipid nanoparticle comprising an endonuclease (e.g., any of the endonucleases disclosed herein) and one or more of any of the guide RNAs disclosed herein. In some embodiments, the vector is a lipid nanoparticle comprising a nucleic acid encoding an endonuclease (e.g., any of the endonucleases disclosed herein) and one or more of any of the guide RNAs disclosed herein. In some embodiments, the vector is a lipid nanoparticle comprising a nucleic acid encoding an endonuclease (e.g., any of the endonucleases disclosed herein) and a nucleic acid encoding one or more of any of the guide RNAs disclosed herein.

Where a vector is used, it may be a viral vector, such as a non-integrating viral vector. In some embodiments, the viral vector is an adeno-associated virus vector, a lentiviral vector, an integrase-deficient lentiviral vector, an adenoviral vector, a vaccinia viral vector, an alphaviral vector, or a herpes simplex viral vector. In some embodiments, the viral vector is an adeno-associated virus (AAV) vector. In some embodiments, the AAV vector is an AAV1, AAV2, AAV3, AAV4, AAV5, AAV6, AAV7, AAV8, AAVrh10 (see, e.g., SEQ ID NO: 81 of U.S. Pat. No. 9,790,472, which is incorporated by reference herein in its entirety), AAVrh74 (see, e.g., SEQ ID NO: 1 of US 2015/0111955, which is incorporated by reference herein in its entirety), or AAV9 vector, wherein the number following AAV indicates the AAV serotype. In some embodiments, the AAV vector is a single-stranded AAV (ssAAV). In some embodiments, the AAV vector is a double-stranded AAV (dsAAV). Any variant of an AAV vector or serotype thereof, such as a self-complementary AAV (scAAV) vector, is encompassed within the general terms AAV vector, AAV1 vector, etc. See, e.g., McCarty et al., Gene Ther. 2001; 8:1248-54, Naso et al., BioDrugs 2017; 31:317-334, and references cited therein for detailed discussion of various AAV vectors. In some embodiments, the AAV vector size is measured in length of nucleotides from ITR to ITR, inclusive of both ITRs. In some embodiments, the AAV vector is less than 5 kb in size from ITR to ITR, inclusive of both ITRs. In particular embodiments, the AAV vector is less than 4.9 kb from ITR to ITR in size, inclusive of both ITRs. In further embodiments, the AAV vector is less than 4.85 kb in size from ITR to ITR, inclusive of both ITRs. In further embodiments, the AAV vector is less than 4.8 kb in size from ITR to ITR, inclusive of both ITRs. In further embodiments, the AAV vector is less than 4.75 kb in size from ITR to ITR, inclusive of both ITRs. In further embodiments, the AAV vector is less than 4.7 kb in size from ITR to ITR, inclusive of both ITRs. In some embodiments, the vector is between 3.9-5 kb, 4-5 kb, 4.2-5 kb, 4.4-5 kb, 4.6-5 kb, 4.7-5 kb, 3.9-4.9 kb, 4.2-4.9 kb, 4.4-4.9 kb, 4.7-4.9 kb, 3.9-4.85 kb, 4.2-4.85 kb, 4.4-4.85 kb, 4.6-4.85 kb, 4.7-4.85 kb, 4.7-4.9 kb, 3.9-4.8 kb, 4.2-4.8 kb, 4.4-4.8 kb or 4.6-4.8 kb from ITR to ITR in size, inclusive of both ITRs. In some embodiments, the vector is between 4.4-4.85 kb in size from ITR to ITR, inclusive of both ITRs. In some embodiments, the vector is an AAV9 vector.

In some embodiments, the vector (e.g., viral vector, such as an adeno-associated viral vector) comprises a tissue-specific (e.g., muscle-specific) promoter, e.g., which is operatively linked to a sequence encoding the guide RNA and/or the Cas protein. In some embodiments, the muscle-specific promoter is a muscle creatine kinase promoter, a desmin promoter, an MHCK7 promoter, or an SPc5-12 promoter. In some embodiments, the muscle-specific promoter is a CK8 promoter. In some embodiments, the muscle-specific promoter is a CK8e promoter. Muscle-specific promoters are described in detail, e.g., in US2004/0175727 A1; Wang et al., Expert Opin Drug Deliv. (2014) 11, 345-364; Wang et al., Gene Therapy (2008) 15, 1489-1499. In some embodiments, the tissue-specific promoter is a neuron-specific promoter, such as an enolase promoter. See, e.g., Naso et al., BioDrugs 2017; 31:317-334; Dashkoff et al., Mol Ther Methods Clin Dev. 2016; 3:16081, and references cited therein for detailed discussion of tissue-specific promoters including neuron-specific promoters.

In some embodiments, in addition to guide RNA and Cas9 sequences, the vectors further comprise nucleic acids that do not encode guide RNAs. Nucleic acids that do not encode guide RNA and Cas9 include, but are not limited to, promoters, enhancers, and regulatory sequences. In some embodiments, the vector comprises one or more nucleotide sequence(s) encoding a crRNA, a trRNA, or a crRNA and trRNA. In some embodiments, the muscle specific promoter is the CK8 promoter. The CK8 promoter has the following sequence (SEQ ID NO. 700):

| | |
|---|---|
| 1 | CTAGACTAGC ATGCTGCCCA TGTAAGGAGG CAAGGCCTGG |
| | GGACACCCGA GATGCCTGGT |
| 61 | TATAATTAAC CCAGACATGT GGCTGCCCCC CCCCCCCCAA |
| | CACCTGCTGC CTCTAAAAAT |
| 121 | AACCCTGCAT GCCATGTTCC CGGCGAAGGG CCAGCTGTCC |
| | CCCGCCAGCT AGACTCAGCA |
| 181 | CTTAGTTTAG GAACCAGTGA GCAAGTCAGC CCTTGGGGCA |
| | GCCCATACAA GGCCATGGGG |
| 241 | CTGGGCAAGC TGCACGCCTG GGTCCGGGGT GGGCACGGTG |
| | CCCGGGCAAC GAGCTGAAAG |
| 301 | CTCATCTGCT CTCAGGGGCC CCTCCCTGGG GACAGCCCCT |
| | CCTGGCTAGT CACACCCTGT |
| 361 | AGGCTCCTCT ATATAACCCA GGGGCACAGG GGCTGCCCTC |
| | ATTCTACCAC CACCTCCACA |
| 421 | GCACAGACAG ACACTCAGGA GCCAGCCAGC |

In some embodiments, the muscle-cell cell specific promoter is a variant of the CK8 promoter, called CK8e. In some embodiments, the size of the CK8e promoter is 436 bp. The CK8e promoter has the following sequence (SEQ ID NO. 701):

| | |
|---|---|
| 1 | TGCCCATGTA AGGAGGCAAG GCCTGGGGAC ACCCGAGATG |
| | CCTGGTTATA ATTAACCCAG |
| 61 | ACATGTGGCT GCCCCCCCCC CCCCAACACC TGCTGCCTCT |
| | AAAAATAACC CTGCATGCCA |
| 121 | TGTTCCCGGC GAAGGGCCAG CTGTCCCCCG CCAGCTAGAC |
| | TCAGCACTTA GTTTAGGAAC |
| 181 | CAGTGAGCAA GTCAGCCCTT GGGGCAGCCC ATACAAGGCC |
| | ATGGGGCTGG GCAAGCTGCA |
| 241 | CGCCTGGGTC CGGGGTGGGC ACGGTGCCCG GGCAACGAGC |
| | TGAAAGCTCA TCTGCTCTCA |
| 301 | GGGGCCCCTC CCTGGGGACA GCCCCTCCTG GCTAGTCACA |
| | CCCTGTAGGC TCCTCTATAT |
| 361 | AACCCAGGGG CACAGGGGCT GCCCTCATTC TACCACCACC |
| | TCCACAGCAC AGACAGACAC |
| 421 | TCAGGAGCCA GCCAGC |

In some embodiments, the Ck8e promoter comprises a nucleotide sequence that is at least 90%, 91%, 92%, 93%, 94%, 95%, 96%, 97%, 98%, 99% or 100% identical to the sequence of SEQ ID NO: 701.

In some embodiments, the vector comprises one or more of a U6, H1, or 7SK promoter. In some embodiments, the U6 promoter is the human U6 promoter (e.g., the U6L promoter or U6S promoter). In some embodiments, the promoter is the murine U6 promoter. In some embodiments, the 7SK promoter is a human 7SK promoter. In some embodiments, the 7SK promoter is the 7SK1 promoter. In some embodiments, the 7SK promoter is the 7SK2 promoter. In some embodiments, the H1 promoter is a human H1 promoter (e.g., the H1L promoter or the HIS promoter). In some embodiments, the vector comprises multiple guide sequences, wherein each guide sequence is under the control of a separate promoter. In some embodiments, each of the multiple guide sequences comprises a different sequence. In some embodiments, each of the multiple guide sequences comprise the same sequence (e.g., each of the multiple guide sequences comprise the same spacer sequence). In some embodiments, each of the multiple guide sequences comprises the same spacer sequence and the same scaffold sequence. In some embodiments, each of the multiple guide sequences comprises different spacer sequences and different scaffold sequences. In some embodiments, each of the multiple guide sequences comprises the same spacer sequence, but comprises a different scaffold sequence. In some embodiments, each of the multiple guide sequences comprises different spacer sequences and different scaffold sequences. In some embodiments, each of the separate promoters comprises the same nucleotide sequence (e.g., the U6 promoter sequence). In some embodiments, each of the separate promoters comprises a different nucleotide sequence (e.g., the U6, H1, and/or 7SK promoter sequence).

In some embodiments, the U6 promoter comprises a nucleotide sequence that is at least 90%, 91%, 92%, 93%, 94%, 95%, 96%, 97%, 98%, 99% or 100% identical to the sequence of SEQ ID NO: 702:

| | |
|---|---|
| cgagtccaac acccgtggga atcccatggg caccatggcc cctcgctcca aaaatgcttt | 60 |
| cgcgtcgcgc agacactgct cggtagtttc ggggatcagc gtttgagtaa gagcccgcgt | 120 |
| ctgaaccctc cgcgccgccc cggccccagt ggaaagacgc gcaggcaaaa cgcaccacgt | 180 |
| gacggagcgt gaccgcgcgc cgagcgcgcg ccaaggtcgg gcaggaagag ggcctatttc | 240 |
| ccatgattcc ttcatatttg catatacgat acaaggctgt tagagagata attagaatta | 300 |
| atttgactgt aaacacaaag atattagtac aaaatacgtg acgtagaaag taataatttc | 360 |
| ttgggtagtt tgcagtttta aaattatgtt ttaaaatgga ctatcatatg cttaccgtaa | 420 |
| cttgaaagta tttcgatttc ttggctttat atatcttgtg gaaaggacga aa | 472 |

In some embodiments, the H1 promoter comprises a nucleotide sequence that is at least 90%, 91%, 92%, 93%, 94%, 95%, 96%, 97%, 98%, 99% or 100% identical to the sequence of SEQ ID NO: 703:

| | |
|---|---|
| gctcggcgcg cccatatttg catgtcgcta tgtgttctgg gaaatcacca taaacgtgaa | 60 |
| atgtctttgg atttgggaat cttataagtt ctgtatgaga ccacggta | 108 |

In some embodiments, the 7SK promoter comprises a nucleotide sequence that is at least 90%, 91%, 92%, 93%, 94%, 95%, 96%, 97%, 98%, 99% or 100% identical to the sequence of SEQ ID NO: 704:

| | |
|---|---|
| tgacggcgcg ccctgcagta tttagcatgc cccacccatc tgcaaggcat tctggatagt | 60 |
| gtcaaaacag ccggaaatca agtccgttta tctcaaactt tagcattttg ggaataaatg | 120 |
| atatttgcta tgctggttaa attagatttt agttaaattt cctgctgaag ctctagtacg | 180 |
| ataagtaact tgacctaagt gtaaagttga gatttccttc aggtttatat agcttgtgcg | 240 |
| ccgcctgggt a | 251 |

In some embodiments, the U6 promoter is a hU6c promoter and comprises a nucleotide sequence that is at least 90%, 91%, 92%, 93%, 94%, 95%, 96%, 97%, 98%, 99% or 100% identical to the sequence of SEQ ID NO: 705:

GAGGGCCTATTTCCCATGATTCCTTCATATTTGCATATACGATACAAGGC
TGTTAGAGAGATAATTGGAATTAATTTGACTGTAAACACAAAGATATTAG
TACAAAATACGTGACGTAGAAAGTAATAATTTCTTGGGTAGTTTGCAGTT
TTAAAATTATGTTTTAAAATGGACTATCATATGCTTACCGTAACTTGAAA
GTATTTCGATTTCTTGGCTTTATATATCTTGTGGAAAGGACGAAACACC.

In some embodiments, the U6 promoter is a variant of the hU6c promoter. In some embodiments, the variant of the hU6c promoter comprises alternative nucleotides as compared to the sequence of SEQ ID NO: 705. In some embodiments, the variant of the hU6c promoter comprises fewer nucleotides as compared to the 249 nucleotides of SEQ ID NO: 705. In some embodiments, the variant of the hU6c promoter has fewer nucleotides in the nucleosome binding sequence of the hU6c promoter of SEQ ID NO: 705. In some embodiments, the variant of the hU6c promoter lacks all of or at least a portion of (e.g., at least 5, 10, 15, 20, 25, or 30 nucleotides) the nucleotides corresponding to nucleotides 96-125 of SEQ ID NO: 705. In some embodiments, the variant of the hU6c promoter lacks all of or at least a portion of (e.g., at least 5, 10, 15, 20, 25, 30, 35, 40, 45, 50, 55, or 60 nucleotides) the nucleotides corresponding to nucleotides 81-140 of SEQ ID NO: 705. In some embodiments, the variant of the hU6c promoter lacks all of or at least a portion of (e.g., at least 10, 20, 30, 40, 50, 60, 65, 70, 75, 80, or 85 nucleotides) the nucleotides corresponding to nucleotides 66-150 of SEQ ID NO: 705. In some embodiments, the variant of the hU6c promoter lacks all of or at least a portion of (e.g., at least 10, 20, 30, 40, 50, 60, 70, 80, 90, 100, 110, or 120 nucleotides) the nucleotides corresponding to nucleotides 51-170 of SEQ ID NO: 705. In some embodiments, the variant of the hU6c promoter lacks the nucleotides corresponding to nucleotides 96-125 of SEQ ID NO: 705. In some embodiments, the variant of the hU6c promoter comprises 129-219 nucleotides. In some embodiments, the variant of the hU6c promoter comprises 219 nucleotides. In some embodiments, the variant of the hU6c promoter comprises 189 nucleotides. In some embodiments, the variant of the hU6c promoter comprises 159 nucleotides. In some embodiments, the variant of the hU6c promoter comprises 129 nucleotides.

In some embodiments, the U6 promoter is hU6d30 and comprises a nucleotide sequence that is at least 90%, 91%, 92%, 93%, 94%, 95%, 96%, 97%, 98%, 99% or 100% identical to the sequence of SEQ ID NO: 9001:

GAGGGCCTATTTCCCATGATTCCTTCATATTTGCATATACGATACAAGGC
TGTTAGAGAGATAATTGGAATTAATTTGACTGTAAACACAAAGATATAAT
TTCTTGGGTAGTTTGCAGTTTTAAAATTATGTTTTAAAATGGACTATCAT
ATGCTTACCGTAACTTGAAAGTATTTCGATTTCTTGGCTTTATATATCTT
GTGGAAAGGACGAAACACC.

In some embodiments, the U6 promoter is hU6d60 and comprises a nucleotide sequence that is at least 90%, 91%, 92%, 93%, 94%, 95%, 96%, 97%, 98%, 99% or 100% identical to the sequence of SEQ ID NO: 9002:

GAGGGCCTATTTCCCATGATTCCTTCATATTTGCATATACGATACAAGGC
TGTTAGAGAGATAATTGGAATTAATTTGACGTTTGCAGTTTTAAAATTAT
GTTTTAAAATGGACTATCATATGCTTACCGTAACTTGAAAGTATTTCGAT
TTCTTGGCTTTATATATCTTGTGGAAAGGACGAAACACC.

In some embodiments, the U6 promoter is hU6d90 and comprises a nucleotide sequence that is at least 90%, 91%, 92%, 93%, 94%, 95%, 96%, 97%, 98%, 99% or 100% identical to the sequence of SEQ ID NO: 9003:

GAGGGCCTATTTCCCATGATTCCTTCATATTTGCATATACGATACAAGGC
TGTTAGAGAGATAATATTATGTTTTAAAATGGACTATCATATGCTTACCG
TAACTTGAAAGTATTTCGATTTCTTGGCTTTATATATCTTGTGGAAAGGA
CGAAACACC.

In some embodiments, the U6 promoter is hU6d120 and comprises a nucleotide sequence that is at least 90%, 91%, 92%, 93%, 94%, 95%, 96%, 97%, 98%, 99% or 100% identical to the sequence of SEQ ID NO: 9004:

GAGGGCCTATTTCCCATGATTCCTTCATATTTGCATATACGATACAAGGC
GGACTATCATATGCTTACCGTAACTTGAAAGTATTTCGATTTCTTGGCTT
TATATATCTTGTGGAAAGGACGAAACACC.

In some embodiments, the 7SK promoter is a 7SK2 promoter and comprises a nucleotide sequence that is at least 90%, 91%, 92%, 93%, 94%, 95%, 96%, 97%, 98%, 99% or 100% identical to the sequence of SEQ ID NO: 706:

CTGCAGTATTTAGCATGCCCCACCCATCTGCAAGGCATTCTGGATAGTGT
CAAAACAGCCGGAAATCAAGTCCGTTTATCTCAAACTTTAGCATTTTGGG
AATAAATGATATTTGCTATGCTGGTTAAATTAGATTTTAGTTAAATTTCC
TGCTGAAGCTCTAGTACGATAAGCAACTTGACCTAAGTGTAAAGTTGAGA
CTTCCTTCAGGTTTATATAGCTTGTGCGCCGCTTGGGTACCTC.

In some embodiments, the 7SK promoter is a variant of the 7SK2 promoter. In some embodiments, the variant of the 7SK2 promoter comprises alternative nucleotides as compared to the sequence of SEQ ID NO: 706. In some embodiments, the variant of the 7SK2 promoter e.g., comprises fewer nucleotides as compared to the 243 nucleotides of SEQ ID NO: 706. In some embodiments, the variant of the 7SK2 promoter has fewer nucleotides in the nucleosome binding sequence of the 7SK2 promoter of SEQ ID NO: 706. In some embodiments, the variant of the 7SK2 promoter lacks all of or at least a portion of (e.g., at least 5, 10, 15, 20, 25, or 30 nucleotides) the nucleotides corresponding to nucleotides 95-124 of SEQ ID NO: 706. In some embodiments, the variant of the 7SK2 promoter lacks all of or at least a portion of (e.g., at least 5, 10, 15, 20, 25, 30, 35, 40, 45, 50, 55, or 60 nucleotides) the nucleotides corresponding to nucleotides 81-140 of SEQ ID NO: 706. In some embodiments, the variant of the 7SK2 promoter lacks all of or at least a portion of (e.g., at least 10, 20, 30, 40, 50, 60, 65, 70, 75, 80, 85 or 90 nucleotides) the nucleotides corresponding to nucleotides 67-156 of SEQ ID NO: 706. In some embodiments, the variant of the 7SK2 promoter lacks all of or at least a portion of (e.g., at least 10, 20, 30, 40, 50, 60, 70, 80, 90, 100, 110, or 120 nucleotides) the nucleotides corresponding to nucleotides 52-171 of SEQ ID NO: 706. In some embodiments, the variant of the 7SK2 promoter comprises 123-213 nucleotides. In some embodiments, the variant of the 7SK2 promoter comprises 213 nucleotides. In some embodiments, the variant of the 7SK2 promoter comprises 183 nucleotides. In some embodiments, the variant of the 7SK2 promoter comprises 153 nucleotides. In some embodiments, the variant of the 7SK2 promoter comprises 123 nucleotides.

In some embodiments, the 7SK promoter is 7SKd30 and comprises a nucleotide sequence that is at least 90%, 91%, 92%, 93%, 94%, 95%, 96%, 97%, 98%, 99% or 100% identical to the sequence of SEQ ID NO: 9006:

CTGCAGTATTTAGCATGCCCCACCCATCTGCAAGGCATTCTGGATAGTGT
CAAAACAGCCGGAAATCAAGTCCGTTTATCTCAAACTTTAGCATTTAAAT
TAGATTTTAGTTAAATTTCCTGCTGAAGCTCTAGTACGATAAGCAACTTG
ACCTAAGTGTAAAGTTGAGACTTCCTTCAGGTTTATATAGCTTGTGCGCC
GCTTGGGTACCTC.

In some embodiments, the 7SK promoter is 7SKd60 and comprises a nucleotide sequence that is at least 90%, 91%, 92%, 93%, 94%, 95%, 96%, 97%, 98%, 99% or 100% identical to the sequence of SEQ ID NO: 9007:

CTGCAGTATTTAGCATGCCCCACCCATCTGCAAGGCATTCTGGATAGTGT
CAAAACAGCCGGAAATCAAGTCCGTTTATCTTAAATTTCCTGCTGAAGCT
CTAGTACGATAAGCAACTTGACCTAAGTGTAAAGTTGAGACTTCCTTCAG
GTTTATATAGCTTGTGCGCCGCTTGGGTACCTC.

In some embodiments, the 7SK promoter is 7SKd90 and comprises a nucleotide sequence that is at least 90%, 91%, 92%, 93%, 94%, 95%, 96%, 97%, 98%, 99% or 100% identical to the sequence of SEQ ID NO: 9008:

CTGCAGTATTTAGCATGCCCCACCCATCTGCAAGGCATTCTGGATAGTGT
CAAAACAGCCGGAAATAGCTCTAGTACGATAAGCAACTTGACCTAAGTGT
AAAGTTGAGACTTCCTTCAGGTTTATATAGCTTGTGCGCCGCTTGGGTAC
CTC.

In some embodiments, the 7SK promoter is 7SKd120 and comprises a nucleotide sequence that is at least 90%, 91%, 92%, 93%, 94%, 95%, 96%, 97%, 98%, 99% or 100% identical to the sequence of SEQ ID NO: 9009:

CTGCAGTATTTAGCATGCCCCACCCATCTGCAAGGCATTCTGGATAGTGTCAG CAACTTGACCTAAGTGTAAAGTTGAGACTTCCTTCAGGTTTATATAGCTTGTGCGCCGCTT GGGTACCTC. In some embodiments, the H1 promoter is a HIm or mHI promoter and comprises a nucleotide sequence that is at least 90%, 91%, 92%, 93%, 94%, 95%, 96%, 97%, 98%, 99% or 100% identical to the sequence of SEQ ID NO: 707:

AATATTTGCATGTCGCTATGTGTTCTGGGAAATCACCATAAACGTGAAAT
GTCTTTGGATTTGGGAATCTTATAAGTTCTGTATGAGACCACTCTTTCC
C.

In some embodiments, the promoter is an M 11 promoter and comprises a nucleotide sequence that is at least 90%, 91%, 92%, 93%, 94%, 95%, 96%, 97%, 98%, 99% or 100% identical to the sequence of SEQ ID NO: 708:

ATATTTAGCATGTCGCTATGTGTTCTGGGAAACTTGACCTAAGTGTAAAG
TTGAGATTTCCTTCAGGTTTATATAGTTCTGTATGAGACCACTCTTTCC
C.

In some embodiments, the vector comprises multiple inverted terminal repeats (ITRs). These ITRs may be of an AAV1, AAV2, AAV3, AAV4, AAV5, AAV6, AAV7, AAV8, or AAV9 serotype. In some embodiments, the ITRs are of an AAV2 serotype. In some embodiments, the 5′ ITR comprises a sequence that is at least 80%, 85%, 90%, 91%, 92%, 93%, 94%, 95%, 96%, 97%, 98%, 99%, or 100% identical to the sequence of SEQ ID NO: 709:

GGCCACTCCCTCTCTGCGCGCTCGCTCGCTCACTGAGGCCGGGCGACCAA
AGGTCGCCCGACGCCCGGGCTTTGCCCGGGCGGCCTCAGTGAGCGAGCGA
GCGCGCAGAGAGGGAGTGGCCAACTCCATCACTAGGGGTTCCT.

In some embodiments, the 5′ ITR comprises a sequence that is at least 80%, 85%, 90%, 91%, 92%, 93%, 94%, 95%, 96%, 97%, 98%, 99%, or 100% identical to the sequence of SEQ ID NO: 939:

CGGCCACTCCCTCTCTGCGCGCTCGCTCGCTCACTGAGGCCGGGC
GACCAAAGGTCGCCCGACGCCCGGGCTTTGCCCGGGCGGCCTCAG
TGAGCGAGCGAGCGCGCAGAGAGGGAGTGGCCAACTCCATCACTA
GGGGTTCCT.

In some embodiments, the 3′ITR comprises a sequence that is at least 80%, 85%, 90%, 91%, 92%, 93%, 94%, 95%, 96%, 97%, 98%, 99%, or 100% identical to the sequence of SEQ ID NO: 710:

AGGAACCCCTAGTGATGGAGTTGGCCACTCCCTCTCTGCGCGCTC
GCTCGCTCACTGAGGCCGGGCGACCAAAGGTCGCCCGACGCCCGG
GCTTTGCCCGGGCGGCCTCAGTGAGCGAGCGAGCGCGCAGAGAGG
GA.

In some embodiments, the 3′ ITR comprises a sequence that is at least 80%, 85%, 90%, 91%, 92%, 93%, 94%, 95%, 96%, 97%, 98%, 99%, or 100% identical to the sequence of SEQ ID NO: 940:

AGGAACCCCTAGTGATGGAGTTGGCCACTCCCTCTCTGCGCGCTC
GCTCGCTCACTGAGGCCGGGCGACCAAAGGTCGCCCGACGCCCGG
GCTTTGCCCGGGCGGCCTCAGTGAGCGAGCGAGCGCGCAGAGAGG
GAA.

In some embodiments, a vector comprising a single nucleic acid molecule encoding 1) two or more guide RNA comprising any one or more of the spacer sequences of Table 6; and 2) a SaCas9 (for any one or more of SEQ ID Nos: 1-159) or SluCas9 (for any one or more of SEQ ID NO: 200-292, 924-938, or 950-955) is provided. In some embodiments, the vector is an AAV vector. In some embodiments, the AAV vector is administered to a subject to treat DMD. In some embodiments, only one vector is needed due to the use of a particular guide sequence that is useful in the context of SaCas9 or SluCas9.

In some embodiments, the vector comprises a nucleic acid encoding a Cas9 protein (e.g., an SaCas9 or SluCas9 protein) and further comprises a nucleic acid encoding one or more single guide RNA(s). In some embodiments, the nucleic acid encoding the Cas9 protein is under the control of a CK8e promoter. In some embodiments, the nucleic acid encoding the guide RNA sequence is under the control of a hU6c promoter. In some embodiments, the vector is AAV9.

In some embodiments, the vector comprises multiple nucleic acids encoding more than one guide RNA. In some embodiments, the vector comprises two nucleic acids encoding two different guide RNA sequences.

In some embodiments, the vector comprises a nucleic acid encoding a Cas9 protein (e.g., an SaCas9 protein or SluCas9 protein), a nucleic acid encoding a first guide RNA, and a nucleic acid encoding a second guide RNA. In some embodiments, the vector does not comprise a nucleic acid encoding more than two guide RNAs. In some embodiments, the nucleic acid encoding the first guide RNA is the same as the nucleic acid encoding the second guide RNA. In some embodiments, the nucleic acid encoding the first guide RNA is different from the nucleic acid encoding the second guide RNA. In some embodiments, the vector comprises a single nucleic acid molecule, wherein the single nucleic acid molecule comprises a nucleic acid encoding a Cas9 protein, a nucleic acid encoding a first guide RNA, and a nucleic acid that is the reverse complement to the coding sequence for the second guide RNA. In some embodiments, the vector comprises a single nucleic acid molecule, wherein the single nucleic acid molecule comprises a nucleic acid encoding a Cas9 protein, a nucleic acid that is the reverse complement to the coding sequence for the first guide RNA, and a nucleic acid that is the reverse complement to the coding sequence for the second guide RNA. In some embodiments, the nucleic acid encoding a Cas9 protein (e.g., an SaCas9 or SluCas9 protein) is under the control of the CK8e promoter. In some embodiments, the first guide is under the control of the hU6c promoter and the second guide is under the control of the hU6c promoter. In some embodiments, the first guide is under the control of the 7SK2 promoter, and the second guide is under the control of the H1m promoter. In some embodiments, the first guide is under the control of the H1m promoter, and the second guide is under the control of the 7SK2 promoter. In some embodiments, the first guide is under the control of the hU6c promoter, and the second guide is under the control of the H1m promoter. In some embodiments, the first guide is under the control of the H1m promoter, and the second guide is under the control of the hU6c promoter. In some embodiments, the nucleic acid encoding the Cas9 protein is: a) between the nucleic acids encoding the guide RNAs, b) between the nucleic acids that are the reverse complement to the coding sequences for the guide RNAs, c) between the nucleic acid encoding the first guide RNA and the nucleic acid that is the reverse complement to the coding sequence for the second guide RNA, d) between the nucleic acid encoding the second guide RNA and the nucleic acid that is the reverse complement to the coding sequence for the first guide RNA, e) 5′ to the nucleic acids encoding the guide RNAs, f) 5′ to the nucleic acids that are the reverse complements to the coding sequences for the guide RNAs, g) 5′ to a nucleic acid encoding one of the guide RNAs and 5′ to a nucleic acid that is the reverse complement to the coding sequence for the other guide RNA, h) 3′ to the nucleic acids encoding the guide RNAs, i) 3′ to the nucleic acids that are the reverse complements to the coding sequences for the guide RNAs, or j) 3′ to a nucleic acid encoding one of the guide RNAs and 3′ to a nucleic acid that is the reverse complement to the coding sequence for the other guide RNA. In some embodiments, any of the vectors disclosed herein is AAV9. In preferred embodiments, the AAV9 vector is less than 5 kb from ITR to ITR in size, inclusive of both ITRs. In particular embodiments, the AAV9 vector is less than 4.9 kb from ITR to ITR in size, inclusive of both ITRs. In further embodiments, the AAV9 vector is less than 4.85 kb from ITR to ITR in size, inclusive of both ITRs. In further embodiments, the AAV9 vector is less than 4.8 kb from ITR to ITR in size, inclusive of both ITRs. In further embodiments, the AAV9 vector is less than 4.75 kb from ITR to ITR in size, inclusive of both ITRs. In further embodiments, the AAV9 vector is less than 4.7 kb from ITR to ITR in size, inclusive of both ITRs. In some embodiments, the vector is between 3.9-5 kb, 4-5 kb, 4.2-5 kb, 4.4-5 kb, 4.6-5 kb, 4.7-5 kb, 3.9-4.9 kb, 4.2-4.9 kb, 4.4-4.9 kb, 4.7-4.9 kb, 3.9-4.85 kb, 4.2-4.85 kb, 4.4-4.85 kb, 4.6-4.85 kb, 4.7-4.85 kb, 4.7-4.9 kb, 3.9-4.8 kb, 4.2-4.8 kb, 4.4-4.8 kb or 4.6-4.8 kb from ITR to ITR in size, inclusive of both ITRs. In some embodiments, the vector is between 4.4-4.85 kb from ITR to ITR in size, inclusive of both ITRs. In some embodiments, the vector is an AAV9 vector.

In some embodiments, any of the vectors disclosed herein comprises a nucleic acid encoding at least a first guide RNA and a second guide RNA. In some embodiments, the nucleic acid comprises a spacer-encoding sequence for the first guide RNA, a scaffold-encoding sequence for the first guide RNA, a spacer-encoding sequence for the second guide RNA, and a scaffold-encoding sequence of the second guide RNA. In some embodiments, the spacer-encoding sequence (e.g., encoding any of the spacer sequences disclosed herein) for the first guide RNA is identical to the spacer-encoding sequence for the second guide RNA. In some embodiments, the spacer-encoding sequence (e.g., encoding any of the spacer sequences disclosed herein) for the first guide RNA is different from the spacer-encoding sequence for the second guide RNA. In some embodiments, the scaffold-encoding sequence for the first guide RNA is identical to the scaffold-encoding sequence for the second guide RNA. In some embodiments, the scaffold-encoding sequence for the first guide RNA is different from the scaffold-encoding sequence for the nucleic acid encoding the second guide RNA. In some embodiments, the scaffold-encoding sequence for the first guide RNA comprises a sequence selected from the group consisting of SEQ ID Nos: 500-504 (for SaCas9) and 601 or 900-917 (for SluCas9), and the scaffold-encoding sequence for the second guide RNA comprises a different sequence selected from the group consisting of SEQ ID Nos: 500-504 (for SaCas9) and 601 or 900-917 (for SluCas9). In some embodiments, the scaffold-encoding sequence for the first guide RNA comprises the sequence of SEQ ID NO: 500, and the scaffold-encoding sequence for the second guide RNA comprises the sequence of SEQ ID NO: 501. In some embodiments, the scaffold-encoding sequence for the first guide RNA comprises the sequence of SEQ ID NO: 500, and the scaffold-encoding sequence for the second guide RNA comprises the sequence of SEQ ID NO: 502. In some embodiments, the scaffold-encoding sequence for the first guide RNA comprises the sequence of SEQ ID NO: 500, and the scaffold-encoding sequence for the second guide RNA comprises the sequence of SEQ ID NO: 503. In some embodiments, the scaffold-encoding sequence for the first guide RNA comprises the sequence of SEQ ID NO: 500, and the scaffold-encoding sequence for the second guide RNA comprises the sequence of SEQ ID NO: 504. In some embodiments, the scaffold-encoding sequence for the first guide RNA comprises the sequence of SEQ ID NO: 501, and the scaffold-encoding sequence for the second guide RNA comprises the sequence of SEQ ID NO: 502. In some embodiments, the scaffold-encoding sequence for the first guide RNA comprises the sequence of SEQ ID NO: 501, and the scaffold-encoding sequence for the second guide RNA comprises the sequence of SEQ ID NO: 503. In some embodiments, the scaffold-encoding sequence for the first guide RNA comprises the sequence of SEQ ID NO: 501, and the scaffold-encoding sequence for the second guide RNA comprises the sequence of SEQ ID NO: 504. In some embodiments, the scaffold-encoding sequence for the first guide RNA comprises the sequence of SEQ ID NO: 502, and the scaffold-encoding sequence for the second guide RNA comprises the sequence of SEQ ID NO: 503. In some embodiments, the scaffold-encoding sequence for the first guide RNA comprises the sequence of SEQ ID NO: 502, and the scaffold-encoding sequence for the second guide RNA comprises the sequence of SEQ ID NO: 504. In some embodiments, the scaffold-encoding sequence for the first guide RNA comprises the sequence of SEQ ID NO: 503, and the scaffold-encoding sequence for the second guide RNA comprises the sequence of SEQ ID NO: 504. In particular embodiments, the scaffold-encoding sequence for the first guide RNA comprises the sequence of SEQ ID NO: 504, and the scaffold-encoding sequence for the second guide RNA comprises the sequence of SEQ ID NO: 504. In some embodiments, the scaffold-encoding sequence for the first guide RNA comprises the sequence of SEQ ID NO: 600, and the scaffold-encoding sequence for the second guide RNA comprises the sequence of SEQ ID NO: 900. In some embodiments, the scaffold-encoding sequence for the first guide RNA comprises the sequence of SEQ ID NO: 600, and the scaffold-encoding sequence for the second guide RNA comprises the sequence of SEQ ID NO: 901. In some embodiments, the scaffold-encoding sequence for the first guide RNA comprises the sequence of SEQ ID NO: 600, and the scaffold-encoding sequence for the second guide RNA comprises the sequence of SEQ ID NO: 902. In some embodiments, the scaffold-encoding sequence for the first guide RNA comprises the sequence of SEQ ID NO: 600, and the scaffold-encoding sequence for the second guide RNA comprises the sequence of SEQ ID NO: 903. In some embodiments, the scaffold-encoding sequence for the first guide RNA comprises the sequence of SEQ ID NO: 600, and the scaffold-encoding sequence for the second guide RNA comprises the sequence of SEQ ID NO: 904. In some embodiments, the scaffold-encoding sequence for the first guide RNA comprises the sequence of SEQ ID NO: 600, and the scaffold-encoding sequence for the second guide RNA comprises the sequence of SEQ ID NO: 905. In some embodiments, the scaffold-encoding sequence for the first guide RNA comprises the sequence of SEQ ID NO: 600, and the scaffold-encoding sequence for the second guide RNA comprises the sequence of SEQ ID NO: 906. In some embodiments, the scaffold-encoding sequence for the first guide RNA comprises the sequence of SEQ ID NO: 600, and the scaffold-encoding sequence for the second guide RNA comprises the sequence of SEQ ID NO: 907. In some embodiments, the scaffold-encoding sequence for the first guide RNA comprises the sequence of SEQ ID NO: 600, and the scaffold-encoding sequence for the second guide RNA comprises the sequence of SEQ ID NO: 908. In some embodiments, the scaffold-encoding sequence for the first guide RNA comprises the sequence of SEQ ID NO: 600, and the scaffold-encoding sequence for the second guide RNA comprises the sequence of SEQ ID NO: 909. In some embodiments, the scaffold-encoding sequence for the first guide RNA comprises the sequence of SEQ ID NO: 600, and the scaffold-encoding sequence for the second guide RNA comprises the sequence of SEQ ID NO: 910. In some embodiments, the scaffold-encoding sequence for the first guide RNA comprises the sequence of SEQ ID NO: 600, and the scaffold-encoding sequence for the second guide RNA comprises the sequence of SEQ ID NO: 911. In some embodiments, the scaffold-encoding sequence for the first guide RNA comprises the sequence of SEQ ID NO: 600, and the scaffold-encoding sequence for the second guide RNA comprises the sequence of SEQ ID NO: 912. In some embodiments, the scaffold-encoding sequence for the first guide RNA comprises the sequence of SEQ ID NO: 600, and the scaffold-encoding sequence for the second guide RNA comprises the sequence of SEQ ID NO: 913. In some embodiments, the scaffold-encoding sequence for the first guide RNA comprises the sequence of SEQ ID NO: 600, and the scaffold-encoding sequence for the second guide RNA comprises the sequence of SEQ ID NO: 914. In some embodiments, the scaffold-encoding sequence for the first guide RNA comprises the sequence of SEQ ID NO: 600, and the scaffold-encoding sequence for the second guide RNA comprises the sequence of SEQ ID NO: 915. In some embodiments, the scaffold-encoding sequence for the first guide RNA comprises the sequence of SEQ ID NO: 600, and the scaffold-encoding sequence for the second guide RNA comprises the sequence of SEQ ID NO: 916. In some embodiments, the scaffold-encoding sequence for the first guide RNA comprises the sequence of SEQ ID NO: 600, and the scaffold-encoding sequence for the second guide RNA comprises the sequence of SEQ ID NO: 917. In some embodiments, the scaffold-encoding sequence for the first guide RNA comprises the sequence of SEQ ID NO: 901, and the scaffold-encoding sequence for the second guide RNA comprises the sequence of SEQ ID NO: 902. In some embodiments, the scaffold-encoding sequence for the first guide RNA comprises the sequence of SEQ ID NO: 901, and the scaffold-encoding sequence for the second guide RNA comprises the sequence of SEQ ID NO: 903. In some embodiments, the scaffold-encoding sequence for the first guide RNA comprises the sequence of SEQ ID NO: 901, and the scaffold-encoding sequence for the second guide RNA comprises the sequence of SEQ ID NO: 904. In some embodiments, the scaffold-encoding sequence for the first guide RNA comprises the sequence of SEQ ID NO: 901, and the scaffold-encoding sequence for the second guide RNA comprises the sequence of SEQ ID NO: 905. In some embodiments, the scaffold-encoding sequence for the first guide RNA comprises the sequence of SEQ ID NO: 901, and the scaffold-encoding sequence for the second guide RNA comprises the sequence of SEQ ID NO: 906. In some embodiments, the scaffold-encoding sequence for the first guide RNA comprises the sequence of SEQ ID NO: 901, and the scaffold-encoding sequence for the second guide RNA comprises the sequence of SEQ ID NO: 907. In some embodiments, the scaffold-encoding sequence for the first guide RNA comprises the sequence of SEQ ID NO: 901, and the scaffold-encoding sequence for the second guide RNA comprises the sequence of SEQ ID NO: 908. In some embodiments, the scaffold-encoding sequence for the first guide RNA comprises the sequence of SEQ ID NO: 901, and the scaffold-encoding sequence for the second guide RNA comprises the sequence of SEQ ID NO: 909. In some embodiments, the scaffold-encoding sequence for the first guide RNA comprises the sequence of SEQ ID NO: 901, and the scaffold-encoding sequence for the second guide RNA comprises the sequence of SEQ ID NO: 910. In some embodiments, the scaffold-encoding sequence for the first guide RNA comprises the sequence of SEQ ID NO: 901, and the scaffold-encoding sequence for the second guide RNA comprises the sequence of SEQ ID NO: 911. In some embodiments, the scaffold-encoding sequence for the first guide RNA comprises the sequence of SEQ ID NO: 901, and the scaffold-encoding sequence for the second guide RNA comprises the sequence of SEQ ID NO: 912. In some embodiments, the scaffold-encoding sequence for the first guide RNA comprises the sequence of SEQ ID NO: 901, and the scaffold-encoding sequence for the second guide RNA comprises the sequence of SEQ ID NO: 913. In some embodiments, the scaffold-encoding sequence for the first guide RNA comprises the sequence of SEQ ID NO: 901, and the scaffold-encoding sequence for the second guide RNA comprises the sequence of SEQ ID NO: 914. In some embodiments, the scaffold-encoding sequence for the first guide RNA comprises the sequence of SEQ ID NO: 901, and the scaffold-encoding sequence for the second guide RNA comprises the sequence of SEQ ID NO: 915. In some embodiments, the scaffold-encoding sequence for the first guide RNA comprises the sequence of SEQ ID NO: 901, and the scaffold-encoding sequence for the second guide RNA comprises the sequence of SEQ ID NO: 916. In some embodiments, the scaffold-encoding sequence for the first guide RNA comprises the sequence of SEQ ID NO: 901, and the scaffold-encoding sequence for the second guide RNA comprises the sequence of SEQ ID NO: 917. In particular embodiments, the scaffold-encoding sequence for the first guide RNA comprises the sequence of SEQ ID NO: 901, and the scaffold-encoding sequence for the second guide RNA comprises the sequence of SEQ ID NO: 901. In some embodiments, the spacer encoding sequence for the first guide RNA is the same as the spacer-encoding sequence in the second guide RNA, and the scaffold-encoding sequence for the first guide RNA is different from the scaffold-encoding sequence in the nucleic acid encoding the second guide RNA.

In some embodiments, the AAV vector is in a particular configuration. Some examples of these AAV vector configurations are provided herein, and the order of elements in these exemplary vectors are referenced in a 5′ to 3′ manner with respect to the plus strand. For these configurations, it should be understood that the recited elements may not be directly contiguous, and that one or more nucleotides or one or more additional elements may be present between the recited elements. However, in some embodiments, it is possible that no nucleotides or no additional elements are present between the recited elements. Also, unless otherwise stated, “a promoter for expression of element X” means that the promoter is oriented in a manner to facilitate expression of the recited element X. In addition, unless otherwise stated, references to an “sgRNA scaffold sequence” or “a guide RNA scaffold sequence” are synonymous with “a nucleotide sequence/nucleic acid encoding an sgRNA scaffold sequence” or “a nucleotide sequence/nucleic acid encoding a guide RNA scaffold sequence.” In some embodiments, the disclosure provides for a nucleic acid encoding an SaCas9 (e.g., an SaCas9-KKH) or SluCas9. In some embodiments, the nucleic acid encodes for one or more nuclear localization signals (e.g., the SV40 NLS and/or the c-Myc NLS) on the C-terminus of the encoded SaCas9 or SluCas9. In some embodiments, the nucleic acid encodes for one or more NLSs (e.g., the SV40 NLS and/or the c-Myc NLS) on the C-terminus of the encoded SaCas9 or SluCas9, and the nucleic acid does not encode for an NLS on the N-terminus of the encoded SaCas9 or SluCas9. In some embodiments, the nucleic acid encodes for one or more nuclear localization signals (e.g., the SV40 NLS and/or the c-Myc NLS) on the N-terminus of the encoded SaCas9 or SluCas9. In some embodiments, the nucleic acid encodes for one or more NLSs (e.g., the SV40 NLS and/or the c-Myc NLS) on the N-terminus of the encoded SaCas9 or SluCas9, and the nucleic acid does not encode for an NLS on the C-terminus of the encoded SaCas9 or SluCas9. In some embodiments, the nucleic acid encodes for one or more nuclear localization signals (e.g., the SV40 NLS and/or the c-Myc NLS) on the C-terminus of the encoded SaCas9 or SluCas9 and also encodes for one or more NLSs on the N-terminus of the encoded SaCas9 or SluCas9 (e.g., the SV40 NLS and/or the c-Myc NLS). In some embodiments, the nucleic acid encodes one NLS. In some embodiments, the nucleic acid encodes two NLSs. In some embodiments, the nucleic acid encodes three NLSs. The one, two, or three NLS may all be C-terminal, N-terminal, or any combination of C- and N-terminal. The NLS may be fused/attached directly to the C- or N-terminus or to another NLS, or may be fused/attached indirectly attached through a linker. In some embodiments, an additional domain may be: a) fused to the N- or C-terminus of the Cas protein (e.g., a Cas9 protein), b) fused to the N-terminus of an NLS fused to the N-terminus of a Cas protein, or c) fused to the C-terminus of an NLS fused to the C-terminus of a Cas protein, with or without a linker. In some embodiments, an NLS is fused to the N- and/or C-terminus of the Cas protein by means of a linker. In some embodiments, an NLS is fused to the N-terminus of an N-terminally-fused NLS on a Cas protein by means of a linker, and/or an NLS is fused to the C-terminus of a C-terminally fused NLS on a Cas protein by means of a linker. In some embodiments, the linker is GSVD (SEQ ID NO: 550) or GSGS (SEQ ID NO: 551). In some embodiments, the Cas protein comprises a c-Myc NLS fused to the N-terminus of the Cas protein (or to an N-terminally-fused NLS on the Cas protein), optionally by means of a linker. In some embodiments, the Cas protein comprises an SV40 NLS fused to the C-terminus of the Cas protein (or to a C-terminally-fused NLS on the Cas protein), optionally by means of a linker. In some embodiments, the Cas protein comprises a nucleoplasmin NLS fused to the C-terminus of the Cas protein (or to a C-terminally-fused NLS on the Cas protein), optionally by means of a linker. In some embodiments, the Cas protein comprises: a) a c-Myc NLS fused to the N-terminus of the Cas protein, optionally by means of a linker, b) an SV40 NLS fused to the C-terminus of the Cas protein, optionally by means of a linker, and c) a nucleoplasmin NLS fused to the C-terminus of the SV40 NLS, optionally by means of a linker. In some embodiments, the Cas protein comprises: a) a c-Myc NLS fused to the N-terminus of the Cas protein, optionally by means of a linker, b) a nucleoplasmin NLS fused to the C-terminus of the Cas protein, optionally by means of a linker, and c) an SV40 NLS fused to the C-terminus of the nucleoplasmin NLS, optionally by means of a linker. In some embodiments, a c-myc NLS is fused to the N-terminus of the Cas and an SV40 NLS and/or nucleoplasmin NLS is fused to the C-terminus of the Cas. In some embodiments, a c-myc NLS is fused to the N-terminus of the Cas (e.g., by means of a linker such as GSVD), an SV40 NLS is fused to the C-terminus of the Cas (e.g., by means of a linker such as GSGS), and a nucleoplasmin NLS is fused to the C-terminus of the SV-40 NLS (e.g., by means of a linker such as GSGS).

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of a promoter for expression of the nucleic acid encoding the first sgRNA, a promoter for expression of a nucleic acid encoding SaCas9 (e.g., CK8e), a nucleic acid encoding SaCas9, a polyadenylation sequence, a promoter for expression of a second sgRNA, a second sgRNA guide sequence, and a second sgRNA scaffold sequence. In some embodiments, the first sgRNA is associated with weaker expression than the second sgRNA when compared individually in a sgRNA expression assay, e.g., in an assay in which each guide is separately assessed (i.e., not in the same construct) using the same promoter, same concentration of genetic material/vector/RNP in substantially the same conditions (e.g., time, pH, temperature, buffer conditions). In some embodiments the promoter for expression of the nucleic acid encoding the first sgRNA is any of the hU6c promoters disclosed herein. In some embodiments the promoter for expression of the nucleic acid encoding the first sgRNA comprises SEQ ID NO: 705. In some embodiments the promoter for expression of the nucleic acid encoding the first sgRNA comprises SEQ ID NO: 9001. In some embodiments the promoter for expression of the nucleic acid encoding the first sgRNA comprises SEQ ID NO: 9002. In some embodiments the promoter for expression of the nucleic acid encoding the first sgRNA comprises SEQ ID NO: 9003. In some embodiments the promoter for expression of the nucleic acid encoding the first sgRNA comprises SEQ ID NO: 9004. In some embodiments the promoter for expression of the nucleic acid encoding the first sgRNA is any of the 7SK2 promoters disclosed herein. In some embodiments the promoter for expression of the nucleic acid encoding the first sgRNA comprises SEQ ID NO: 705. In some embodiments the promoter for expression of the nucleic acid encoding the first sgRNA comprises SEQ ID NO: 9006. In some embodiments the promoter for expression of the nucleic acid encoding the first sgRNA comprises SEQ ID NO: 9007. In some embodiments the promoter for expression of the nucleic acid encoding the first sgRNA comprises SEQ ID NO: 9008. In some embodiments the promoter for expression of the nucleic acid encoding the first sgRNA comprises SEQ ID NO: 9009. In some embodiments the promoter for expression of the nucleic acid encoding the second sgRNA is any of the hU6c promoters disclosed herein. In some embodiments the promoter for expression of the nucleic acid encoding the second sgRNA comprises SEQ ID NO: 705. In some embodiments the promoter for expression of the nucleic acid encoding the second sgRNA comprises SEQ ID NO: 9001. In some embodiments the promoter for expression of the nucleic acid encoding the second sgRNA comprises SEQ ID NO: 9002. In some embodiments the promoter for expression of the nucleic acid encoding the second sgRNA comprises SEQ ID NO: 9003. In some embodiments the promoter for expression of the nucleic acid encoding the second sgRNA comprises SEQ ID NO: 9004. In some embodiments the promoter for expression of the nucleic acid encoding the second sgRNA is any of the 7SK2 promoters disclosed herein. In some embodiments the promoter for expression of the nucleic acid encoding the second sgRNA comprises SEQ ID NO: 705. In some embodiments the promoter for expression of the nucleic acid encoding the second sgRNA comprises SEQ ID NO: 9006. In some embodiments the promoter for expression of the nucleic acid encoding the second sgRNA comprises SEQ ID NO: 9007. In some embodiments the promoter for expression of the nucleic acid encoding the second sgRNA comprises SEQ ID NO: 9008. In some embodiments the promoter for expression of the nucleic acid encoding the second sgRNA comprises SEQ ID NO: 9009. In some embodiments the promoter for expression of the nucleic acid encoding the second sgRNA is any of the H1m promoters disclosed herein. In some embodiments, the promoter for SaCas9 is the CK8e promoter. In some embodiments, the nucleic acid sequence encoding SaCas9 is fused to a nucleic acid sequence encoding a nuclear localization sequence (NLS). In some embodiments, the nucleic acid sequence encoding SaCas9 is fused to two nucleic acid sequences each encoding a nuclear localization sequence (NLS). In some embodiments, the nucleic acid sequence encoding SaCas9 is fused to three nucleic acid sequences each encoding a nuclear localization sequence (NLS). In some embodiments, the one or more NLSs is an SV40 NLS. In some embodiments, the one or more NLSs is a c-Myc NLS. In some embodiments, the one or more NLSs is a nucleoplasmin NLS. In some embodiments, the NLS is fused to the SaCas9 with a linker.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of an hU6c promoter for expression of the nucleic acid encoding the first sgRNA, a promoter for expression of a nucleic acid encoding SaCas9 (e.g., CK8e), a nucleic acid encoding SaCas9, a polyadenylation sequence, an hU6c promoter for expression of a second sgRNA, a second sgRNA guide sequence, and a second sgRNA scaffold sequence. In some embodiments, the first sgRNA and the second sgRNA are selected from Table 6. In some embodiments, the nucleic acid sequence encoding SaCas9 is fused to a nucleic acid sequence encoding a nuclear localization sequence (NLS). In some embodiments, the nucleic acid sequence encoding SaCas9 is fused to two nucleic acid sequences each encoding a nuclear localization sequence (NLS). In some embodiments, the nucleic acid sequence encoding SaCas9 is fused to three nucleic acid sequences each encoding a nuclear localization sequence (NLS). In some embodiments, the one or more NLSs is an SV40 NLS. In some embodiments, the one or more NLSs is a c-Myc NLS. In some embodiments, the NLS is fused to the SaCas9 with a linker.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of an hU6c promoter for expression of the nucleic acid encoding the first sgRNA, a promoter for expression of a nucleic acid encoding SaCas9 (e.g., CK8e), a nucleic acid encoding SaCas9, a polyadenylation sequence, an 7SK promoter for expression of a second sgRNA, a second sgRNA guide sequence, and a second sgRNA scaffold sequence. In some embodiments, the first sgRNA and the second sgRNA are selected from Table 6. In some embodiments, the nucleic acid sequence encoding SaCas9 is fused to a nucleic acid sequence encoding a nuclear localization sequence (NLS). In some embodiments, the nucleic acid sequence encoding SaCas9 is fused to two nucleic acid sequences each encoding a nuclear localization sequence (NLS). In some embodiments, the nucleic acid sequence encoding SaCas9 is fused to three nucleic acid sequences each encoding a nuclear localization sequence (NLS). In some embodiments, the one or more NLSs is an SV40 NLS. In some embodiments, the one or more NLSs is a c-Myc NLS. In some embodiments, the NLS is fused to the SaCas9 with a linker.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of an hU6c promoter for expression of the nucleic acid encoding the first sgRNA, a promoter for expression of a nucleic acid encoding Cas9 (e.g., CK8e), a nucleic acid encoding Cas9, a polyadenylation sequence, an H1m promoter for expression of a second sgRNA, a second sgRNA guide sequence, and a second sgRNA scaffold sequence. In some embodiments, the first sgRNA and the second sgRNA are selected from Table 6. In some embodiments, the nucleic acid sequence encoding Cas9 is fused to a nucleic acid sequence encoding a nuclear localization sequence (NLS). In some embodiments, the nucleic acid sequence encoding Cas9 is fused to two nucleic acid sequences each encoding a nuclear localization sequence (NLS). In some embodiments, the nucleic acid sequence encoding Cas9 is fused to three nucleic acid sequences each encoding a nuclear localization sequence (NLS). In some embodiments, the one or more NLSs is an SV40 NLS. In some embodiments, the one or more NLSs is a c-Myc NLS. In some embodiments, the NLS is fused to the Cas9 with a linker.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of an 7SK promoter for expression of the nucleic acid encoding the first sgRNA, a promoter for expression of a nucleic acid encoding Cas9 (e.g., CK8e), a nucleic acid encoding Cas9, a polyadenylation sequence, an H1m promoter for expression of a second sgRNA, a second sgRNA guide sequence, and a second sgRNA scaffold sequence. In some embodiments, the first sgRNA and the second sgRNA are selected from Table 6. In some embodiments, the nucleic acid sequence encoding Cas9 is fused to a nucleic acid sequence encoding a nuclear localization sequence (NLS). In some embodiments, the nucleic acid sequence encoding Cas9 is fused to two nucleic acid sequences each encoding a nuclear localization sequence (NLS). In some embodiments, the nucleic acid sequence encoding Cas9 is fused to three nucleic acid sequences each encoding a nuclear localization sequence (NLS). In some embodiments, the one or more NLSs is an SV40 NLS. In some embodiments, the one or more NLSs is a c-Myc NLS. In some embodiments, the NLS is fused to the Cas9 with a linker.

In some embodiments, the disclosure provides for a composition comprising at least two nucleic acids. In some embodiments, the composition comprises at least two nucleic acid molecules, wherein the first nucleic acid molecule comprises a sequence encoding any of the endonucleases disclosed herein (e.g., a SaCas9, SluCas9, or a sRGN), wherein the second nucleic acid molecule encodes a first guide RNA and a second guide RNA, wherein the first guide RNA and the second guide RNA are not the same sequence, and wherein the second nucleic acid molecule does not encode an endonuclease. In some embodiments, the first nucleic acid molecule also encodes a copy of the first guide RNA and a copy of the second guide RNA. In some embodiments, the first nucleic acid molecule does not encode any guide RNAs. In some embodiments, the second nucleic acid molecule encodes two copies of the first guide RNA and two copies of the second guide RNA. In some embodiments, the second nucleic molecule encodes two copies of the first guide RNA, and one copy of the second guide RNA. In some embodiments, the second nucleic acid molecule encodes one copy of the first guide RNA, and two copies of the second guide RNA. In some embodiments, the second nucleic acid molecule comprises two copies of the first guide RNA, and three copies of the second guide RNA. In some embodiments, the second nucleic acid molecule comprises three copies of the first guide RNA, and two copies of the second guide RNA. In some embodiments, the second nucleic acid does not encode a Cas protein. In some embodiments, the second nucleic acid molecule encodes three copies of the first guide RNA and three copies of the second guide RNA. In some embodiments, the first nucleic acid molecule comprises from 5′ to 3′ with respect to the plus strand: the reverse complement of a first guide RNA scaffold sequence, the reverse complement of a nucleotide sequence encoding the first guide RNA sequence, the reverse complement of a promoter for expression of the nucleotide sequence encoding the first guide RNA sequence, a promoter for expression of a nucleotide sequence encoding the endonuclease, a nucleotide sequence encoding an endonuclease, a polyadenylation sequence, a promoter for expression of the second guide RNA in the same direction as the promoter for the endonuclease, the second guide RNA sequence, and a second guide RNA scaffold sequence. In some embodiments, the promoter for expression of the nucleotide sequence encoding the first guide RNA sequence in the first nucleic acid molecule is a U6 promoter and the promoter for expression of the nucleotide sequence encoding the second guide RNA in the first nucleic acid molecule is a U6 promoter.

In some embodiments, the AAV vector comprises from 5′ to 3′ with respect to the plus strand: a promoter for expression of a nucleic acid encoding a first sgRNA, a nucleic acid encoding the first sgRNA guide sequence, the first sgRNA scaffold sequence, a promoter for expression of Cas9 (e.g., CK8e), a nucleic acid encoding Cas9, a polyadenylation sequence, a promoter for expression of a second sgRNA, the second sgRNA guide sequence, and a second sgRNA scaffold sequence. See FIG. 5A at “Design 1”.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of a promoter for expression of the nucleic acid encoding the first sgRNA, a promoter for expression of a nucleic acid encoding Cas9 (e.g., CK8e), a nucleic acid encoding Cas9, a polyadenylation sequence, a promoter for expression of a second sgRNA, a second sgRNA guide sequence, and a second sgRNA scaffold sequence. See FIG. 5A at “Design 2”.

In some embodiments, the AAV vector comprises from 5′ to 3′ with respect to the plus strand: a promoter for expression of a nucleic acid encoding a first sgRNA, a nucleic acid encoding the first sgRNA guide sequence, a first sgRNA scaffold sequence, a promoter for expression of a second sgRNA, a second sgRNA guide sequence, and a second sgRNA scaffold sequence, a promoter for Cas9 (e.g., CK8e), a nucleic acid encoding Cas9, and a polyadenylation sequence. See FIG. 5A at “Design 3”.

In some embodiments, the AAV vector comprises from 5′ to 3′ with respect to the plus strand: a promoter for expression of a nucleic acid encoding Cas9 (e.g., CK8e), a nucleic acid encoding Cas9, a polyadenylation sequence, a promoter for expression of the nucleic acid encoding a first guide RNA, a nucleic acid encoding the first sgRNA guide sequence, a first sgRNA scaffold sequence, a promoter for expression of the second sgRNA, a second sgRNA guide sequence, and a second sgRNA scaffold sequence. See FIG. 5A at “Design 4”.

In some embodiments, the AAV vector comprises from 5′ to 3′ with respect to the plus strand: a first sgRNA scaffold sequence (e.g., V5), a nucleic acid encoding a first sgRNA guide sequence (e.g., Slu3 or Slu7), a promoter (e.g., U6) for expression of the nucleic acid encoding the first sgRNA guide sequence, a promoter (e.g., CK8e) for expression (in the opposite direction of expression of the nucleic acid encoding the first sgRNA guide sequence) of a nucleic acid encoding a SluCas9, a nucleic acid encoding a first NLS, a nucleic acid encoding a SluCas9, a nucleic acid encoding a second NLS, a nucleic acid encoding a third NLS, a promoter (e.g., U6) for expression (in the same direction as expression of the nucleic acid encoding the SluCas9) of a second sgRNA guide sequence (e.g., Slu 7 or Slu 3), a nucleic acid encoding the second sgRNA guide sequence, and a second sgRNA scaffold sequence (e.g., V5). See FIG. 5B at “Design 5” and “Design 6”. Designs 5 and 6 are specific versions of Design 2, as described herein.

In a particular embodiment, the AAV vector comprises from 5′ to 3′ with respect to the plus strand: the reverse complement of a first V5 scaffold sequence, the reverse complement of a nucleic acid encoding a Slu3 guide RNA sequence selected from SEQ ID NOs: 217 or 950-955, the reverse complement of a U6 promoter for expression of the nucleic acid encoding the Slu3 guide RNA sequence, a CK8e promoter for expression (in the opposite direction of expression of the nucleic acid encoding the Slu3 sgRNA guide sequence) of a nucleic acid encoding a SluCas9, a nucleic acid encoding a c-Myc NLS, a nucleic acid encoding a SluCas9, a nucleic acid encoding a SV40 NLS, a nucleic acid encoding a NP NLS, a U6 promoter for expression (in the same direction as expression of the nucleic acid encoding the SluCas9) of a Slu7 guide RNA sequence of SEQ ID NO: 275, a nucleic acid encoding the Slu7 guide RNA sequence, and a second V5 scaffold sequence. See FIG. 5B at “Design 5” and “Design 6”. Designs 5 and 6 are specific versions of Design 2, as described herein.

In another particular embodiment, the AAV vector comprises from 5′ to 3′ with respect to the plus strand: the reverse complement of a first V5 scaffold sequence, the reverse complement of a nucleic acid encoding a Slu7 guide RNA sequence of SEQ ID NO: 275, the reverse complement of a U6 promoter for expression of the nucleic acid encoding the Slu7 guide RNA sequence, a CK8e promoter for expression (in the opposite direction of expression of the nucleic acid encoding the Slu7 sgRNA guide sequence) of a nucleic acid encoding a SluCas9, a nucleic acid encoding a c-Myc NLS, a nucleic acid encoding a SluCas9, a nucleic acid encoding a SV40 NLS, a nucleic acid encoding a NP NLS, a U6 promoter for expression (in the same direction as expression of the nucleic acid encoding the SluCas9) of a Slu3 guide RNA sequence selected from SEQ ID NOs: 217 or 950-955, a nucleic acid encoding the Slu3 guide RNA sequence, and a second V5 scaffold sequence. See FIG. 5B at “Design 5” and “Design 6”. Designs 5 and 6 are specific versions of Design 2, as described herein.

In some embodiments, the first nucleic acid molecule is in a first vector (e.g., AAV9), and the second nucleic acid is in a separate second vector. In some embodiments, the first vector is AAV9. In some embodiments, the second vector is AAV9. In preferred embodiments, the AAV9 vector is less than 5 kb from ITR to ITR in size, inclusive of both ITRs. In particular embodiments, the AAV9 vector is less than 4.9 kb from ITR to ITR in size, inclusive of both ITRs. In further embodiments, the AAV9 vector is less than 4.85 kb from ITR to ITR in size, inclusive of both ITRs. In further embodiments, the AAV9 vector is less than 4.8 kb from ITR to ITR in size, inclusive of both ITRs. In further embodiments, the AAV9 vector is less than 4.75 kb from ITR to ITR in size, inclusive of both ITRs. In further embodiments, the AAV9 vector is less than 4.7 kb from ITR to ITR in size, inclusive of both ITRs. In some embodiments, the second vector comprises from 5′ to 3′ with respect to the plus strand: a promoter for expression of a first copy of a first guide RNA (e.g., a U6 promoter), a first copy of a nucleotide sequence encoding a first guide RNA, a first copy of a nucleotide sequence encoding a first guide RNA scaffold, a promoter for expression of a second copy of the first guide RNA (e.g., a H1 promoter), a second copy of the nucleotide sequence encoding the first guide RNA, a second copy of the nucleotide sequence encoding the first guide RNA scaffold, a promoter for expression of a second guide RNA (e.g., a 7SK promoter), a nucleotide sequence encoding a second guide RNA, and a nucleotide sequence encoding a second guide RNA scaffold. In some embodiments, the second vector comprises from 5′ to 3′ with respect to the plus strand: a promoter for expression of a first guide RNA (e.g., a U6 promoter), a nucleotide sequence encoding a first guide RNA, a nucleotide sequence encoding a first guide RNA scaffold, a promoter for expression of a second guide RNA (e.g., a 7SK promoter), a nucleotide sequence encoding a second guide RNA, and a nucleotide sequence encoding a second guide RNA scaffold. In some embodiments, the second vector comprises a stuffer sequence (e.g., a 3′UTR desmin sequence) between the nucleotide sequence encoding the first guide scaffold sequence and the promoter for expression of the second guide sequence. In some embodiments, the second vector comprises from 5′ to 3′ with respect to the plus strand: the reverse complement of a nucleotide sequence encoding a scaffold for a first guide RNA, the reverse complement of a nucleotide sequence encoding a first guide RNA, the reverse complement of a promoter for expression of the first guide RNA (e.g., a U6c promoter), a promoter for expression of a second guide RNA (e.g., a U6c promoter), a nucleotide sequence encoding a second guide RNA, and a nucleotide sequence encoding a second guide RNA scaffold. In some embodiments, the second vector comprises a stuffer sequence (e.g., a 3′UTR desmin sequence) between the reverse complement of the promoter for expression of the first guide RNA and the promoter for expression of the second guide RNA. In some embodiments, the second vector comprises from 5′ to 3′ with respect to the plus strand: the reverse complement of a nucleotide sequence encoding a first copy of a first guide RNA scaffold, the reverse complement of a nucleotide sequence encoding a first copy of the first guide RNA, the reverse complement of a promoter for expression of the first copy of the first guide RNA (e.g., a 7SK2 promoter), the reverse complement of a second copy of the nucleotide sequence encoding the first guide RNA scaffold, the reverse complement of a second copy of the nucleotide sequence encoding the first guide RNA, the reverse complement of a promoter for expression of the second copy of the nucleotide sequence encoding the first guide RNA (e.g., a hU6c promoter), a promoter for expression of a first copy of a second guide RNA (e.g., a hU6c promoter), a first copy of a nucleotide sequence encoding a second guide RNA, a first copy of a nucleotide sequence encoding a second guide RNA scaffold, a promoter for expression of a second copy of the second guide RNA (e.g., a 7Sk2 promoter), a second copy of the nucleotide sequence encoding the second guide RNA, and a second copy of the nucleotide sequence encoding the second guide RNA scaffold. In some embodiments, the second vector comprises a stuffer sequence (e.g., a 3′UTR desmin sequence) between the reverse complement of the promoter for expression of the second copy of the first guide RNA and the promoter for expression of the first copy of the second guide RNA. In some embodiments, the second vector comprises from 5′ to 3′ with respect to the plus strand: the reverse complement of a nucleotide sequence encoding a first copy of a first guide RNA scaffold, the reverse complement of a first copy of a nucleotide sequence encoding the first guide RNA, the reverse complement of a promoter for expression of the first copy of the first guide RNA (e.g., a 7SK2 promoter), the reverse complement of a first copy of a nucleotide sequence encoding a second guide RNA scaffold, the reverse complement of a nucleotide sequence encoding the first copy of the second guide RNA, the reverse complement of a promoter for expression of the first copy of the second guide RNA (e.g., a hU6c promoter), a promoter for expression of a second copy of the second guide RNA (e.g., a hU6c promoter), a second copy of the nucleotide sequence encoding the second guide RNA, a second copy of the nucleotide sequence encoding the second guide RNA scaffold, a promoter for expression of a second copy of the first guide RNA (e.g., a 7SK2 promoter), a second copy of the nucleotide sequence encoding the first guide RNA, and a second copy of the nucleotide sequence encoding the first guide RNA scaffold. In some embodiments, the second vector comprises a stuffer sequence (e.g., a 3′UTR desmin sequence) between the reverse complement of the promoter for expression of the first copy of the second guide RNA and the promoter for expression of the second copy of the first guide RNA. In some embodiments, the second vector comprises from 5′ to 3′ with respect to the plus strand: the reverse complement of a nucleotide sequence encoding a first guide RNA scaffold, the reverse complement of a nucleotide sequence encoding a first guide RNA, the reverse complement of a promoter for expression of a first guide RNA (e.g., a hU6c promoter), a promoter for expression of a second guide RNA (e.g., a hU6c promoter), a nucleotide sequence encoding a second guide RNA, and a nucleotide sequence encoding a second guide RNA scaffold. In some embodiments, the second vector comprises a stuffer sequence (e.g., a 3′UTR desmin sequence) between the reverse complement of the promoter for expression of the first guide RNA and the promoter for expression of the second guide RNA. In particular embodiments, the first guide RNA is different from the second guide RNA. In some embodiments, the first guide RNA comprises a sequence of Table 6 and the second guide RNA comprises a different sequence from Table 6.

In some embodiments, if the composition comprises one or more nucleic acids encoding an RNA-targeted endonuclease and one or more guide RNAs, the one or more nucleic acids are designed such that they express the one or more guide RNAs at an equivalent or higher level (e.g., a greater number of expressed transgene copies) as compared to the expression level of the RNA-targeted endonuclease. In some embodiments, the one or more nucleic acids are designed such that they express (e.g., on average in 100 cells) the one or more guide RNAs at least a 1.1, 1.2, 1.3, 1.4, or 1.5 times higher level (e.g., a greater number of expressed transgene copies) as compared to the expression level of the RNA-targeted endonuclease. In some embodiments, the one or more nucleic acids are designed such that they express the one or more guide RNAs at 1.01-1.5, 1.01-1.4, 1.01-1.3, 1.01-1.2, 1.01-1.1, 1.1-2.0, 1.1-1.8, 1.1-1.6, 1.1-1.4, 1.1-1.3, 1.2-2.0, 1.2-1.8, 1.2-1.6, 1.2-1.4, 1.4-2.0, 1.4-1.8, 1.4-1.6, 1.6-2.0, 1.6-1.8, or 1.8-2.0 times higher level (e.g., a greater number of expressed transgene copies) as compared to the expression level of the RNA-targeted endonuclease. In some embodiments, the one or more guide RNAs are designed to express a higher level than the RNA-targeted endonuclease by: a) utilizing one or more regulatory elements (e.g., promoters or enhancers) that express the one or more guide RNAs at a higher level as compared to the regulatory elements (e.g., promoters or enhancers) for expression of the RNA-targeted endonuclease; and/or b) expressing more copies of one or more of the guide RNAs as compared to the number of copies of the RNA-targeted endonuclease (e.g., 2× or 3× as many copies of the nucleotide sequences encoding the one or more guide RNAs as compared to the number of copies of the nucleotide sequences encoding the RNA-targeted endonuclease). For example, in some embodiments, the composition comprises multiple nucleic acid molecules (e.g., in multiple vectors), wherein for every nucleotide sequence encoding an RNA-targeted endonuclease in the nucleic acid molecules in the composition, there are two or three copies of the nucleotide sequence encoding the guide RNA in the nucleic acid molecules in the composition. In some embodiments, the composition comprises a first guide RNA and a second guide RNA, wherein the first guide RNA and the second guide RNA are not the same (e.g., any of the guide RNA pairs disclosed herein), and for every nucleotide sequence encoding an RNA-targeted endonuclease in the nucleic acid molecules in the composition, there are two or three copies of the nucleotide sequence encoding the first guide RNA and/or the second guide RNA.

### Endonucleases

In some embodiments, any of the nucleic acids disclosed herein encodes an RNA-targeted endonuclease. In some embodiments, the RNA-targeted endonuclease has cleavase activity, which can also be referred to as double-strand endonuclease activity. In some embodiments, the RNA-targeted endonuclease comprises a Cas nuclease. Examples of Cas9 nucleases include those of the type II CRISPR systems.

In some embodiments, the Cas protein comprises an amino acid sequence that is at least 80%, 85%, 90%, 91%, 92%, 93%, 94%, 95%, 96%, 97%, 98%, 99% or 100% identical to the sequence of SEQ ID NO: 941 (designated herein as SpCas9):

MDKKYSIGLDIGTNSVGWAVITDEYKVPSKKFKVLGNTDRHSIKK
NLIGALLFDSGETAEATRLKRTARRRYTRRKNRICYLQEIFSNEM
AKVDDSFFHRLEESFLVEEDKKHERHPIFGNIVDEVAYHEKYPTI
YHLRKKLVDSTDKADLRLIYLALAHMIKFRGHFLIEGDLNPDNSD
VDKLFIQLVQTYNQLFEENPINASGVDAKAILSARLSKSRRLENL
IAQLPGEKKNGLFGNLIALSLGLTPNFKSNFDLAEDAKLQLSKDT
YDDDLDNLLAQIGDQYADLFLAAKNLSDAILLSDILRVNTEITKA
PLSASMIKRYDEHHQDLTLLKALVRQQLPEKYKEIFFDQSKNGYA
GYIDGGASQEEFYKFIKPILEKMDGTEELLVKLNREDLLRKQRTF
DNGSIPHQIHLGELHAILRRQEDFYPFLKDNREKIEKILTFRIPY
YVGPLARGNSRFAWMTRKSEETITPWNFEEVVDKGASAQSFIERM
TNFDKNLPNEKVLPKHSLLYEYFTVYNELTKVKYVTEGMRKPAFL
SGEQKKAIVDLLFKTNRKVTVKQLKEDYFKKIECFDSVEISGVED
RFNASLGTYHDLLKIIKDKDFLDNEENEDILEDIVLTLTLFEDRE
MIEERLKTYAHLFDDKVMKQLKRRRYTGWGRLSRKLINGIRDKQS
GKTILDFLKSDGFANRNFMQLIHDDSLTFKEDIQKAQVSGQGDSL
HEHIANLAGSPAIKKGILQTVKVVDELVKVMGRHKPENIVIEMAR
ENQTTQKGQKNSRERMKRIEEGIKELGSQILKEHPVENTQLQNEK
LYLYYLQNGRDMYVDQELDINRLSDYDVDHIVPQSFLKDDSIDNK
VLTRSDKNRGKSDNVPSEEVVKKMKNYWRQLLNAKLITQRKFDNL
TKAERGGLSELDKAGFIKRQLVETRQITKHVAQILDSRMNTKYDE
NDKLIREVKVITLKSKLVSDFRKDFQFYKVREINNYHHAHDAYLN
AVVGTALIKKYPKLESEFVYGDYKVYDVRKMIAKSEQEIGKATAK
YFFYSNIMNFFKTEITLANGEIRKRPLIETNGETGEIVWDKGRDF
ATVRKVLSMPQVNIVKKTEVQTGGFSKESILPKRNSDKLIARKKD
WDPKKYGGFDSPTVAYSVLVVAKVEKGKSKKLKSVKELLGITIME
RSSFEKNPIDFLEAKGYKEVKKDLIIKLPKYSLFELENGRKRMLA
SAGELQKGNELALPSKYVNFLYLASHYEKLKGSPEDNEQKQLFVE
QHKHYLDEIIEQISEFSKRVILADANLDKVLSAYNKHRDKPIREQ
AENIIHLFTLTNLGAPAAFKYFDTTIDRKRYTSTKEVLDATLIHQ
SITGLYETRIDLSQLGGD.

In some embodiments, the nucleic acid encoding SaCas9 encodes an SaCas9 comprising an amino acid sequence that is at least 80%, 85%, 90%, 91%, 92%, 93%, 94%, 95%, 96%, 97%, 98%, 99% or 100% identical to the sequence of SEQ ID NO: 711:

KRNYILGLDIGITSVGYGIIDYETRDVIDAGVRLFKEANVENNEG
RRSKRGARRLKRRRRHRIQRVKKLLFDYNLLTDHSELSGINPYEA
RVKGLSQKLSEEEFSAALLHLAKRRGVHNVNEVEEDTGNELSTKE
QISRNSKALEEKYVAELQLERLKKDGEVRGSINRFKTSDYVKEAK
QLLKVQKAYHQLDQSFIDTYIDLLETRRTYYEGPGEGSPFGWKDI
KEWYEMLMGHCTYFPEELRSVKYAYNADLYNALNDLNNLVITRDE
NEKLEYYEKFQIIENVFKQKKKPTLKQIAKEILVNEEDIKGYRVT
STGKPEFTNLKVYHDIKDITARKEIIENAELLDQIAKILTIYQSS
EDIQEELTNLNSELTQEEIEQISNLKGYTGTHNLSLKAINLILDE
LWHTNDNQIAIFNRLKLVPKKVDLSQQKEIPTTLVDDFILSPVVK
RSFIQSIKVINAIIKKYGLPNDIIIELAREKNSKDAQKMINEMQK
RNRQTNERIEEIIRTTGKENAKYLIEKIKLHDMQEGKCLYSLEAI
PLEDLLNNPFNYEVDHIIPRSVSFDNSFNNKVLVKQEENSKKGNR
TPFQYLSSSDSKISYETFKKHILNLAKGKGRISKTKKEYLLEERD
INRFSVQKDFINRNLVDTRYATRGLMNLLRSYFRVNNLDVKVKSI
NGGFTSFLRRKWKFKKERNKGYKHHAEDALIIANADFIFKEWKKL
DKAKKVMENQMFEEKQAESMPEIETEQEYKEIFITPHQIKHIKDF
KDYKYSHRVDKKPNRELINDTLYSTRKDDKGNTLIVNNLNGLYDK
DNDKLKKLINKSPEKLLMYHHDPQTYQKLKLIMEQYGDEKNPLYK
YYEETGNYLTKYSKKDNGPVIKKIKYYGNKLNAHLDITDDYPNSR
NKVVKLSLKPYRFDVYLDNGVYKFVTVKNLDVIKKENYYEVNSKC
YEEAKKLKKISNQAEFIASFYNNDLIKINGELYRVIGVNNDLLNR
IEVNMIDITYREYLENMNDKRPPRIIKTIASKTQSIKKYSTDILG
NLYEVKSKKHPQIIKKG.

In some embodiments, the nucleic acid encoding SaCas9 comprises the nucleic acid of SEQ ID NO: 9014:

AAGCGCAATTACATCCTGGGCCTGGATATCGGCATCACCTCCGTG
GGCTACGGCATCATCGACTATGAGACACGGGATGTGATCGACGCC
GGCGTGAGACTGTTCAAGGAGGCCAACGTGGAGAACAATGAGGGC
CGGCGGAGCAAGAGGGGAGCAAGGCGCCTGAAGCGGAGAAGGCGC
CACAGAATCCAGAGAGTGAAGAAGCTGCTGTTCGATTACAACCTG
CTGACCGACCACTCCGAGCTGTCTGGCATCAATCCTTATGAGGCC
CGGGTGAAGGGCCTGTCCCAGAAGCTGTCTGAGGAGGAGTTTTCT
GCCGCCCTGCTGCACCTGGCAAAGAGGAGAGGCGTGCACAACGTG
AATGAGGTGGAGGAGGACACCGGCAACGAGCTGAGCACAAAGGAG
CAGATCAGCCGCAATTCCAAGGCCCTGGAGGAGAAGTATGTGGCC
GAGCTGCAGCTGGAGCGGCTGAAGAAGGATGGCGAGGTGAGGGGC
TCCATCAATCGCTTCAAGACCTCTGACTACGTGAAGGAGGCCAAG
CAGCTGCTGAAGGTGCAGAAGGCCTACCACCAGCTGGATCAGAGC
TTTATCGATACATATATCGACCTGCTGGAGACCAGGCGCACATAC
TATGAGGGACCAGGAGAGGGCTCCCCCTTCGGCTGGAAGGACATC
AAGGAGTGGTACGAGATGCTGATGGGCCACTGCACCTATTTTCCA
GAGGAGCTGAGATCCGTGAAGTACGCCTATAACGCCGATCTGTAC
AACGCCCTGAATGACCTGAACAACCTGGTCATCACCAGGGATGAG
AACGAGAAGCTGGAGTACTATGAGAAGTTCCAGATCATCGAGAAC
GTGTTCAAGCAGAAGAAGAAGCCTACACTGAAGCAGATCGCCAAG
GAGATCCTGGTGAACGAGGAGGACATCAAGGGCTACCGCGTGACC
AGCACAGGCAAGCCAGAGTTCACCAATCTGAAGGTGTATCACGAT
ATCAAGGACATCACAGCCCGGAAGGAGATCATCGAGAACGCCGAG
CTGCTGGATCAGATCGCCAAGATCCTGACCATCTATCAGAGCTCC
GAGGACATCCAGGAGGAGCTGACCAACCTGAATAGCGAGCTGACA
CAGGAGGAGATCGAGCAGATCAGCAATCTGAAGGGCTACACCGGC
ACACACAACCTGTCCCTGAAGGCCATCAATCTGATCCTGGATGAG
CTGTGGCACACAAACGACAATCAGATCGCCATCTTTAACAGGCTG
AAGCTGGTGCCAAAGAAGGTGGACCTGAGCCAGCAGAAGGAGATC
CCAACCACACTGGTGGACGATTTCATCCTGTCCCCCGTGGTGAAG
CGGAGCTTCATCCAGAGCATCAAAGTGATCAACGCCATCATCAAG
AAGTACGGCCTGCCCAATGATATCATCATCGAGCTGGCCAGGGAG
AAGAACTCTAAGGACGCCCAGAAGATGATCAATGAGATGCAGAAG
AGGAACCGCCAGACCAATGAGCGGATCGAGGAGATCATCAGAACC
ACAGGCAAGGAGAACGCCAAGTACCTGATCGAGAAGATCAAGCTG
CACGATATGCAGGAGGGCAAGTGTCTGTATAGCCTGGAGGCCATC
CCTCTGGAGGACCTGCTGAACAATCCATTCAACTACGAGGTGGAT
CACATCATCCCCCGGAGCGTGAGCTTCGACAATTCCTTTAACAAT
AAGGTGCTGGTGAAGCAGGAGGAGAACTCTAAGAAGGGCAATAGG
ACCCCTTTCCAGTACCTGTCTAGCTCCGATTCTAAGATCAGCTAC
GAGACCTTCAAGAAGCACATCCTGAATCTGGCCAAGGGCAAGGGC
CGCATCTCTAAGACCAAGAAGGAGTACCTGCTGGAGGAGCGGGAC
ATCAACAGATTCAGCGTGCAGAAGGACTTCATCAACCGGAATCTG
GTGGACACCAGATACGCCACACGCGGCCTGATGAATCTGCTGCGG
TCCTATTTCAGAGTGAACAATCTGGATGTGAAGGTGAAGAGCATC
AACGGCGGCTTCACCTCCTTTCTGCGGAGAAAGTGGAAGTTTAAG
AAGGAGAGAAACAAGGGCTATAAGCACCACGCCGAGGATGCCCTG
ATCATCGCCAATGCCGACTTCATCTTTAAGGAGTGGAAGAAGCTG
GACAAGGCCAAGAAAGTGATGGAGAACCAGATGTTCGAGGAGAAG
CAGGCCGAGAGCATGCCCGAGATCGAGACCGAGCAGGAGTACAAG
GAGATTTTCATCACACCTCACCAGATCAAGCACATCAAGGACTTC
AAGGACTACAAGTATTCCCACAGGGTGGATAAGAAGCCCAACCGC
GAGCTGATCAATGACACCCTGTATTCTACAAGGAAGGACGATAAG
GGCAATACCCTGATCGTGAACAATCTGAACGGCCTGTACGACAAG
GATAATGACAAGCTGAAGAAGCTGATCAACAAGAGCCCCGAGAAG
CTGCTGATGTACCACCACGATCCTCAGACATATCAGAAGCTGAAG
CTGATCATGGAGCAGTACGGCGACGAGAAGAACCCACTGTATAAG
TACTATGAGGAGACCGGCAACTACCTGACAAAGTATTCCAAGAAG
GATAATGGCCCCGTGATCAAGAAGATCAAGTACTATGGCAACAAG
CTGAATGCCCACCTGGACATCACCGACGATTACCCCAACAGCCGG
AATAAGGTGGTGAAGCTGAGCCTGAAGCCATACAGGTTCGACGTG
TACCTGGACAACGGCGTGTATAAGTTTGTGACAGTGAAGAATCTG
GATGTGATCAAGAAGGAGAACTACTATGAAGTGAATAGCAAGTGC
TACGAGGAGGCCAAGAAGCTGAAGAAGATCAGCAACCAGGCCGAG
TTCATCGCCTCTTTTTACAACAATGACCTGATCAAGATCAATGGC
GAGCTGTATAGAGTGATCGGCGTGAACAATGATCTGCTGAACCGC
ATCGAAGTGAATATGATCGACATCACCTACCGGGAGTATCTGGAG
AACATGAATGATAAGAGGCCCCCTCGCATCATCAAGACCATCGCC
TCTAAGACACAGAGCATCAAGAAGTACTCTACAGACATCCTGGGC
AACCTGTATGAGGTGAAGAGCAAGAAGCACCCTCAGATCATCAAG
AAGGGC.

In some embodiments comprising a nucleic acid encoding SaCas9, the SaCas9 comprises an amino acid sequence of SEQ ID NO: 711.

In some embodiments, the SaCas9 is a variant of the amino acid sequence of SEQ ID NO: 711. In some embodiments, the SaCas9 comprises an amino acid other than an E at the position corresponding to position 781 of SEQ ID NO: 711. In some embodiments, the SaCas9 comprises an amino acid other than an N at the position corresponding to position 967 of SEQ ID NO: 711. In some embodiments, the SaCas9 comprises an amino acid other than an R at the position corresponding to position 1014 of SEQ ID NO: 711. In some embodiments, the SaCas9 comprises a K at the position corresponding to position 781 of SEQ ID NO: 711. In some embodiments, the SaCas9 comprises a K at the position corresponding to position 967 of SEQ ID NO: 711. In some embodiments, the SaCas9 comprises an H at the position corresponding to position 1014 of SEQ ID NO: 711. In some embodiments, the SaCas9 comprises an amino acid other than an E at the position corresponding to position 781 of SEQ ID NO: 711; an amino acid other than an N at the position corresponding to position 967 of SEQ ID NO: 711; and an amino acid other than an R at the position corresponding to position 1014 of SEQ ID NO: 711. In some embodiments, the SaCas9 comprises a K at the position corresponding to position 781 of SEQ ID NO: 711; a K at the position corresponding to position 967 of SEQ ID NO: 711; and an H at the position corresponding to position 1014 of SEQ ID NO: 711.

In some embodiments, the SaCas9 comprises an amino acid other than an R at the position corresponding to position 244 of SEQ ID NO: 711. In some embodiments, the SaCas9 comprises an amino acid other than an N at the position corresponding to position 412 of SEQ ID NO: 711. In some embodiments, the SaCas9 comprises an amino acid other than an N at the position corresponding to position 418 of SEQ ID NO: 711. In some embodiments, the SaCas9 comprises an amino acid other than an R at the position corresponding to position 653 of SEQ ID NO: 711. In some embodiments, the SaCas9 comprises an amino acid other than an R at the position corresponding to position 244 of SEQ ID NO: 711; an amino acid other than an N at the position corresponding to position 412 of SEQ ID NO: 711; an amino acid other than an N at the position corresponding to position 418 of SEQ ID NO: 711; and an amino acid other than an R at the position corresponding to position 653 of SEQ ID NO: 711. In some embodiments, the SaCas9 comprises an A at the position corresponding to position 244 of SEQ ID NO: 711. In some embodiments, the SaCas9 comprises an A at the position corresponding to position 412 of SEQ ID NO: 711. In some embodiments, the SaCas9 comprises an A at the position corresponding to position 418 of SEQ ID NO: 711. In some embodiments, the SaCas9 comprises an A at the position corresponding to position 653 of SEQ ID NO: 711. In some embodiments, the SaCas9 comprises an A at the position corresponding to position 244 of SEQ ID NO: 711; an A at the position corresponding to position 412 of SEQ ID NO: 711; an A at the position corresponding to position 418 of SEQ ID NO: 711; and an A at the position corresponding to position 653 of SEQ ID NO: 711.

In some embodiments, the SaCas9 comprises an amino acid other than an R at the position corresponding to position 244 of SEQ ID NO: 711; an amino acid other than an N at the position corresponding to position 412 of SEQ ID NO: 711; an amino acid other than an N at the position corresponding to position 418 of SEQ ID NO: 711; an amino acid other than an R at the position corresponding to position 653 of SEQ ID NO: 711; an amino acid other than an E at the position corresponding to position 781 of SEQ ID NO: 711; an amino acid other than an N at the position corresponding to position 967 of SEQ ID NO: 711; and an amino acid other than an R at the position corresponding to position 1014 of SEQ ID NO: 711. In some embodiments, the SaCas9 comprises an A at the position corresponding to position 244 of SEQ ID NO: 711; an A at the position corresponding to position 412 of SEQ ID NO: 711; an A at the position corresponding to position 418 of SEQ ID NO: 711; an A at the position corresponding to position 653 of SEQ ID NO: 711; a K at the position corresponding to position 781 of SEQ ID NO: 711; a K at the position corresponding to position 967 of SEQ ID NO: 711; and an H at the position corresponding to position 1014 of SEQ ID NO: 711.

In some embodiments, the SaCas9 comprises an amino acid sequence that is at least 80%, 85%, 90%, 91%, 92%, 93%, 94%, 95%, 96%, 97%, 98%, 99% or 100% identical to the sequence of SEQ ID NO: 715 (designated herein as SaCas9-KKH or SACAS9KKH):

KRNYILGLDIGITSVGYGIIDYETRDVIDAGVRLFKEANVENNEG
RRSKRGARRLKRRRRHRIQRVKKLLFDYNLLTDHSELSGINPYEA
RVKGLSQKLSEEEFSAALLHLAKRRGVHNVNEVEEDTGNELSTKE
QISRNSKALEEKYVAELQLERLKKDGEVRGSINRFKTSDYVKEAK
QLLKVQKAYHQLDQSFIDTYIDLLETRRTYYEGPGEGSPFGWKDI
KEWYEMLMGHCTYFPEELRSVKYAYNADLYNALNDLNNLVITRDE
NEKLEYYEKFQIIENVFKQKKKPTLKQIAKEILVNEEDIKGYRVT
STGKPEFTNLKVYHDIKDITARKEIIENAELLDQIAKILTIYQSS
EDIQEELTNLNSELTQEEIEQISNLKGYTGTHNLSLKAINLILDE
LWHTNDNQIAIFNRLKLVPKKVDLSQQKEIPTTLVDDFILSPVVK
RSFIQSIKVINAIIKKYGLPNDIIIELAREKNSKDAQKMINEMQK
RNRQTNERIEEIIRTTGKENAKYLIEKIKLHDMQEGKCLYSLEAI
PLEDLLNNPFNYEVDHIIPRSVSFDNSFNNKVLVKQEENSKKGNR
TPFQYLSSSDSKISYETFKKHILNLAKGKGRISKTKKEYLLEERD
INRFSVQKDFINRNLVDTRYATRGLMNLLRSYFRVNNLDVKVKSI
NGGFTSFLRRKWKFKKERNKGYKHHAEDALIIANADFIFKEWKKL
DKAKKVMENQMFEEKQAESMPEIETEQEYKEIFITPHQIKHIKDF
KDYKYSHRVDKKPNRKLINDTLYSTRKDDKGNTLIVNNLNGLYDK
DNDKLKKLINKSPEKLLMYHHDPQTYQKLKLIMEQYGDEKNPLYK
YYEETGNYLTKYSKKDNGPVIKKIKYYGNKLNAHLDITDDYPNSR
NKVVKLSLKPYRFDVYLDNGVYKFVTVKNLDVIKKENYYEVNSKC
YEEAKKLKKISNQAEFIASFYKNDLIKINGELYRVIGVNNDLLNR
IEVNMIDITYREYLENMNDKRPPHIIKTIASKTQSIKKYSTDILG
NLYEVKSKKHPQIIKKG.

In some embodiments, the SaCas9 comprises an amino acid sequence that is at least 80%, 85%, 90%, 91%, 92%, 93%, 94%, 95%, 96%, 97%, 98%, 99% or 100% identical to the sequence of SEQ ID NO: 716 (designated herein as SaCas9-HF):

KRNYILGLDIGITSVGYGIIDYETRDVIDAGVRLFKEANVENNEG
RRSKRGARRLKRRRRHRIQRVKKLLFDYNLLTDHSELSGINPYEA
RVKGLSQKLSEEEFSAALLHLAKRRGVHNVNEVEEDTGNELSTKE
QISRNSKALEEKYVAELQLERLKKDGEVRGSINRFKTSDYVKEAK
QLLKVQKAYHQLDQSFIDTYIDLLETRRTYYEGPGEGSPFGWKDI
KEWYEMLMGHCTYFPEELASVKYAYNADLYNALNDLNNLVITRDE
NEKLEYYEKFQIIENVFKQKKKPTLKQIAKEILVNEEDIKGYRVT
STGKPEFTNLKVYHDIKDITARKEIIENAELLDQIAKILTIYQSS
EDIQEELTNLNSELTQEEIEQISNLKGYTGTHNLSLKAINLILDE
LWHTNDAQIAIFARLKLVPKKVDLSQQKEIPTTLVDDFILSPVVK
RSFIQSIKVINAIIKKYGLPNDIIIELAREKNSKDAQKMINEMQK
RNRQTNERIEEIIRTTGKENAKYLIEKIKLHDMQEGKCLYSLEAI
PLEDLLNNPFNYEVDHIIPRSVSFDNSFNNKVLVKQEENSKKGNR
TPFQYLSSSDSKISYETFKKHILNLAKGKGRISKTKKEYLLEERD
INRFSVQKDFINRNLVDTRYATAGLMNLLRSYFRVNNLDVKVKSI
NGGFTSFLRRKWKFKKERNKGYKHHAEDALIIANADFIFKEWKKL
DKAKKVMENQMFEEKQAESMPEIETEQEYKEIFITPHQIKHIKDF
KDYKYSHRVDKKPNRELINDTLYSTRKDDKGNTLIVNNLNGLYDK
DNDKLKKLINKSPEKLLMYHHDPQTYQKLKLIMEQYGDEKNPLYK
YYEETGNYLTKYSKKDNGPVIKKIKYYGNKLNAHLDITDDYPNSR
NKVVKLSLKPYRFDVYLDNGVYKFVTVKNLDVIKKENYYEVNSKC
YEEAKKLKKISNQAEFIASFYNNDLIKINGELYRVIGVNNDLLNR
IEVNMIDITYREYLENMNDKRPPRIIKTIASKTQSIKKYSTDILG
NLYEVKSKKHPQIIKKG.

In some embodiments, the SaCas9 comprises an amino acid sequence that is at least 80%, 85%, 90%, 91%, 92%, 93%, 94%, 95%, 96%, 97%, 98%, 99% or 100% identical to the sequence of SEQ ID NO: 717 (designated herein as SaCas9-KKH-HF):

KRNYILGLDIGITSVGYGIIDYETRDVIDAGVRLFKEANVENNEG
RRSKRGARRLKRRRRHRIQRVKKLLFDYNLLTDHSELSGINPYEA
RVKGLSQKLSEEEFSAALLHLAKRRGVHNVNEVEEDTGNELSTKE
QISRNSKALEEKYVAELQLERLKKDGEVRGSINRFKTSDYVKEAK
QLLKVQKAYHQLDQSFIDTYIDLLETRRTYYEGPGEGSPFGWKDI
KEWYEMLMGHCTYFPEELASVKYAYNADLYNALNDLNNLVITRDE
NEKLEYYEKFQIIENVFKQKKKPTLKQIAKEILVNEEDIKGYRVT
STGKPEFTNLKVYHDIKDITARKEIIENAELLDQIAKILTIYQSS
EDIQEELTNLNSELTQEEIEQISNLKGYTGTHNLSLKAINLILDE
LWHTNDAQIAIFARLKLVPKKVDLSQQKEIPTTLVDDFILSPVVK
RSFIQSIKVINAIIKKYGLPNDIIIELAREKNSKDAQKMINEMQK
RNRQTNERIEEIIRTTGKENAKYLIEKIKLHDMQEGKCLYSLEAI
PLEDLLNNPFNYEVDHIIPRSVSFDNSFNNKVLVKQEENSKKGNR
TPFQYLSSSDSKISYETFKKHILNLAKGKGRISKTKKEYLLEERD
INRFSVQKDFINRNLVDTRYATAGLMNLLRSYFRVNNLDVKVKSI
NGGFTSFLRRKWKFKKERNKGYKHHAEDALIIANADFIFKEWKKL
DKAKKVMENQMFEEKQAESMPEIETEQEYKEIFITPHQIKHIKDF
KDYKYSHRVDKKPNRKLINDTLYSTRKDDKGNTLIVNNLNGLYDK
DNDKLKKLINKSPEKLLMYHHDPQTYQKLKLIMEQYGDEKNPLYK
YYEETGNYLTKYSKKDNGPVIKKIKYYGNKLNAHLDITDDYPNSR
NKVVKLSLKPYRFDVYLDNGVYKFVTVKNLDVIKKENYYEVNSKC
YEEAKKLKKISNQAEFIASFYKNDLIKINGELYRVIGVNNDLLNR
IEVNMIDITYREYLENMNDKRPPHIIKTIASKTQSIKKYSTDILG
NLYEVKSKKHPQIIKKG.

In some embodiments, the nucleic acid encoding SluCas9 encodes a SluCas9 comprising an amino acid sequence that is at least 80%, 85%, 90%, 91%, 92%, 93%, 94%, 95%, 96%, 97%, 98%, 99% or 100% identical to the sequence of SEQ ID NO: 712:

NQKFILGLDIGITSVGYGLIDYETKNIIDAGVRLFPEANVENNEG
RRSKRGSRRLKRRRIHRLERVKKLLEDYNLLDQSQIPQSTNPYAI
RVKGLSEALSKDELVIALLHIAKRRGIHKIDVIDSNDDVGNELST
KEQLNKNSKLLKDKFVCQIQLERMNEGQVRGEKNRFKTADIIKEI
IQLLNVQKNFHQLDENFINKYIELVEMRREYFEGPGKGSPYGWEG
DPKAWYETLMGHCTYFPDELRSVKYAYSADLFNALNDLNNLVIQR
DGLSKLEYHEKYHIIENVFKQKKKPTLKQIANEINVNPEDIKGYR
ITKSGKPQFTEFKLYHDLKSVLFDQSILENEDVLDQIAEILTIYQ
DKDSIKSKLTELDILLNEEDKENIAQLTGYTGTHRLSLKCIRLVL
EEQWYSSRNQMEIFTHLNIKPKKINLTAANKIPKAMIDEFILSPV
VKRTFGQAINLINKIIEKYGVPEDIIIELARENNSKDKQKFINEM
QKKNENTRKRINEIIGKYGNQNAKRLVEKIRLHDEQEGKCLYSLE
SIPLEDLLNNPNHYEVDHIIPRSVSFDNSYHNKVLVKQSENSKKS
NLTPYQYFNSGKSKLSYNQFKQHILNLSKSQDRISKKKKEYLLEE
RDINKFEVQKEFINRNLVDTRYATRELTNYLKAYFSANNMNVKVK
TINGSFTDYLRKVWKFKKERNHGYKHHAEDALIIANADFLFKENK
KLKAVNSVLEKPEIETKQLDIQVDSEDNYSEMFIIPKQVQDIKDF
RNFKYSHRVDKKPNRQLINDTLYSTRKKDNSTYIVQTIKDIYAKD
NTTLKKQFDKSPEKFLMYQHDPRTFEKLEVIMKQYANEKNPLAKY
HEETGEYLTKYSKKNNGPIVKSLKYIGNKLGSHLDVTHQFKSSTK
KLVKLSIKPYRFDVYLTDKGYKFITISYLDVLKKDNYYYIPEQKY
DKLKLGKAIDKNAKFIASFYKNDLIKLDGEIYKIIGVNSDTRNMI
ELDLPDIRYKEYCELNNIKGEPRIKKTIGKKVNSIEKLTTDVLGN
VFTNTQYTKPQLLFKRGN.

In some embodiments, the SluCas9 is a variant of the amino acid sequence of SEQ ID NO: 712. In some embodiments, the SluCas9 comprises an amino acid other than an Q at the position corresponding to position 781 of SEQ ID NO: 712. In some embodiments, the SluCas9 comprises an amino acid other than an R at the position corresponding to position 1013 of SEQ ID NO: 712. In some embodiments, the SluCas9 comprises a K at the position corresponding to position 781 of SEQ ID NO: 712. In some embodiments, the SluCas9 comprises a K at the position corresponding to position 966 of SEQ ID NO: 712. In some embodiments, the SluCas9 comprises an H at the position corresponding to position 1013 of SEQ ID NO: 712. In some embodiments, the SluCas9 comprises an amino acid other than an Q at the position corresponding to position 781 of SEQ ID NO: 712; and an amino acid other than an R at the position corresponding to position 1013 of SEQ ID NO: 712. In some embodiments, the SluCas9 comprises a K at the position corresponding to position 781 of SEQ ID NO: 712; a K at the position corresponding to position 966 of SEQ ID NO: 712; and an H at the position corresponding to position 1013 of SEQ ID NO: 712.

In some embodiments, the SluCas9 comprises an amino acid other than an R at the position corresponding to position 246 of SEQ ID NO: 712. In some embodiments, the SluCas9 comprises an amino acid other than an N at the position corresponding to position 414 of SEQ ID NO: 712. In some embodiments, the SluCas9 comprises an amino acid other than a T at the position corresponding to position 420 of SEQ ID NO: 712. In some embodiments, the SluCas9 comprises an amino acid other than an R at the position corresponding to position 655 of SEQ ID NO: 712. In some embodiments, the SluCas9 comprises an amino acid other than an R at the position corresponding to position 246 of SEQ ID NO: 712; an amino acid other than an N at the position corresponding to position 414 of SEQ ID NO: 712; an amino acid other than a T at the position corresponding to position 420 of SEQ ID NO: 712; and an amino acid other than an R at the position corresponding to position 655 of SEQ ID NO: 712. In some embodiments, the SluCas9 comprises an A at the position corresponding to position 246 of SEQ ID NO: 712. In some embodiments, the SluCas9 comprises an A at the position corresponding to position 414 of SEQ ID NO: 712. In some embodiments, the SluCas9 comprises an A at the position corresponding to position 420 of SEQ ID NO: 712. In some embodiments, the SluCas9 comprises an A at the position corresponding to position 655 of SEQ ID NO: 712. In some embodiments, the SluCas9 comprises an A at the position corresponding to position 246 of SEQ ID NO: 712; an A at the position corresponding to position 414 of SEQ ID NO: 712; an A at the position corresponding to position 420 of SEQ ID NO: 712; and an A at the position corresponding to position 655 of SEQ ID NO: 712.

In some embodiments, the SluCas9 comprises an amino acid other than an R at the position corresponding to position 246 of SEQ ID NO: 712; an amino acid other than an N at the position corresponding to position 414 of SEQ ID NO: 712; an amino acid other than a T at the position corresponding to position 420 of SEQ ID NO: 712; an amino acid other than an R at the position corresponding to position 655 of SEQ ID NO: 712; an amino acid other than an Q at the position corresponding to position 781 of SEQ ID NO: 712; a K at the position corresponding to position 966 of SEQ ID NO: 712; and an amino acid other than an R at the position corresponding to position 1013 of SEQ ID NO: 712. In some embodiments, the SluCas9 comprises an A at the position corresponding to position 246 of SEQ ID NO: 712; an A at the position corresponding to position 414 of SEQ ID NO: 712; an A at the position corresponding to position 420 of SEQ ID NO: 712; an A at the position corresponding to position 655 of SEQ ID NO: 712; a K at the position corresponding to position 781 of SEQ ID NO: 712; a K at the position corresponding to position 966 of SEQ ID NO: 712; and an H at the position corresponding to position 1013 of SEQ ID NO: 712.

In some embodiments, the SluCas9 comprises an amino acid sequence that is at least 80%, 85%, 90%, 91%, 92%, 93%, 94%, 95%, 96%, 97%, 98%, 99% or 100% identical to the sequence of SEQ ID NO: 718 (designated herein as SluCas9-KH or SLUCAS9KH):

NQKFILGLDIGITSVGYGLIDYETKNIIDAGVRLFPEANVENNEG
RRSKRGSRRLKRRRIHRLERVKKLLEDYNLLDQSQIPQSTNPYAI
RVKGLSEALSKDELVIALLHIAKRRGIHKIDVIDSNDDVGNELST
KEQLNKNSKLLKDKFVCQIQLERMNEGQVRGEKNRFKTADIIKEI
IQLLNVQKNFHQLDENFINKYIELVEMRREYFEGPGKGSPYGWEG
DPKAWYETLMGHCTYFPDELRSVKYAYSADLFNALNDLNNLVIQR
DGLSKLEYHEKYHIIENVFKQKKKPTLKQIANEINVNPEDIKGYR
ITKSGKPQFTEFKLYHDLKSVLFDQSILENEDVLDQIAEILTIYQ
DKDSIKSKLTELDILLNEEDKENIAQLTGYTGTHRLSLKCIRLVL
EEQWYSSRNQMEIFTHLNIKPKKINLTAANKIPKAMIDEFILSPV
VKRTFGQAINLINKIIEKYGVPEDIIIELARENNSKDKQKFINEM
QKKNENTRKRINEIIGKYGNQNAKRLVEKIRLHDEQEGKCLYSLE
SIPLEDLLNNPNHYEVDHIIPRSVSFDNSYHNKVLVKQSENSKKS
NLTPYQYFNSGKSKLSYNQFKQHILNLSKSQDRISKKKKEYLLEE
RDINKFEVQKEFINRNLVDTRYATRELTNYLKAYFSANNMNVKVK
TINGSFTDYLRKVWKFKKERNHGYKHHAEDALIIANADFLFKENK
KLKAVNSVLEKPEIETKQLDIQVDSEDNYSEMFIIPKQVQDIKDF
RNFKYSHRVDKKPNRKLINDTLYSTRKKDNSTYIVQTIKDIYAKD
NTTLKKQFDKSPEKFLMYQHDPRTFEKLEVIMKQYANEKNPLAKY
HEETGEYLTKYSKKNNGPIVKSLKYIGNKLGSHLDVTHQFKSSTK
KLVKLSIKPYRFDVYLTDKGYKFITISYLDVLKKDNYYYIPEQKY
DKLKLGKAIDKNAKFIASFYKNDLIKLDGEIYKIIGVNSDTRNMI
ELDLPDIRYKEYCELNNIKGEPHIKKTIGKKVNSIEKLTTDVLGN
VFTNTQYTKPQLLFKRGN.

In some embodiments, the SluCas9 comprises an amino acid sequence that is at least 80%, 85%, 90%, 91%, 92%, 93%, 94%, 95%, 96%, 97%, 98%, 99% or 100% identical to the sequence of SEQ ID NO: 719 (designated herein as SluCas9-HF):

NQKFILGLDIGITSVGYGLIDYETKNIIDAGVRLFPEANVENNEGR
RSKRGSRRLKRRRIHRLERVKKLLEDYNLLDQSQIPQSTNPYAIR
VKGLSEALSKDELVIALLHIAKRRGIHKIDVIDSNDDVGNELSTK
EQLNKNSKLLKDKFVCQIQLERMNEGQVRGEKNRFKTADIIKEII
QLLNVQKNFHQLDENFINKYIELVEMRREYFEGPGKGSPYGWEGD
PKAWYETLMGHCTYFPDELASVKYAYSADLFNALNDLNNLVIQRD
GLSKLEYHEKYHIIENVFKQKKKPTLKQIANEINVNPEDIKGYRI
TKSGKPQFTEFKLYHDLKSVLFDQSILENEDVLDQIAEILTIYQD
KDSIKSKLTELDILLNEEDKENIAQLTGYTGTHRLSLKCIRLVLE
EQWYSSRAQMEIFAHLNIKPKKINLTAANKIPKAMIDEFILSPVV
KRTFGQAINLINKIIEKYGVPEDIIIELARENNSKDKQKFINEMQ
KKNENTRKRINEIIGKYGNQNAKRLVEKIRLHDEQEGKCLYSLES
IPLEDLLNNPNHYEVDHIIPRSVSFDNSYHNKVLVKQSENSKKSN
LTPYQYFNSGKSKLSYNQFKQHILNLSKSQDRISKKKKEYLLEER
DINKFEVQKEFINRNLVDTRYATAELTNYLKAYFSANNMNVKVKT
INGSFTDYLRKVWKFKKERNHGYKHHAEDALIIANADFLFKENKK
LKAVNSVLEKPEIETKQLDIQVDSEDNYSEMFIIPKQVQDIKDFR
NFKYSHRVDKKPNRQLINDTLYSTRKKDNSTYIVQTIKDIYAKDN
TTLKKQFDKSPEKFLMYQHDPRTFEKLEVIMKQYANEKNPLAKYH
EETGEYLTKYSKKNNGPIVKSLKYIGNKLGSHLDVTHQFKSSTKK
LVKLSIKPYRFDVYLTDKGYKFITISYLDVLKKDNYYYIPEQKYD
KLKLGKAIDKNAKFIASFYKNDLIKLDGEIYKIIGVNSDTRNMIE
LDLPDIRYKEYCELNNIKGEPRIKKTIGKKVNSIEKLTTDVLGNV
FTNTQYTKPQLLFKRGN.

In some embodiments, the SluCas9 comprises an amino acid sequence that is at least 80%, 85%, 90%, 91%, 92%, 93%, 94%, 95%, 96%, 97%, 98%, 99% or 100% identical to the sequence of SEQ ID NO: 720 (designated herein as SluCas9-HF-KH):

NQKFILGLDIGITSVGYGLIDYETKNIIDAGVRLFPEANVENNEG
RRSKRGSRRLKRRRIHRLERVKKLLEDYNLLDQSQIPQSTNPYAI
RVKGLSEALSKDELVIALLHIAKRRGIHKIDVIDSNDDVGNELST
KEQLNKNSKLLKDKFVCQIQLERMNEGQVRGEKNRFKTADIIKEI
IQLLNVQKNFHQLDENFINKYIELVEMRREYFEGPGKGSPYGWEG
DPKAWYETLMGHCTYFPDELASVKYAYSADLFNALNDLNNLVIQR
DGLSKLEYHEKYHIIENVFKQKKKPTLKQIANEINVNPEDIKGYR
ITKSGKPQFTEFKLYHDLKSVLFDQSILENEDVLDQIAEILTIYQ
DKDSIKSKLTELDILLNEEDKENIAQLTGYTGTHRLSLKCIRLVL
EEQWYSSRAQMEIFAHLNIKPKKINLTAANKIPKAMIDEFILSPV
VKRTFGQAINLINKIIEKYGVPEDIIIELARENNSKDKQKFINEM
QKKNENTRKRINEIIGKYGNQNAKRLVEKIRLHDEQEGKCLYSLE
SIPLEDLLNNPNHYEVDHIIPRSVSFDNSYHNKVLVKQSENSKKS
NLTPYQYFNSGKSKLSYNQFKQHILNLSKSQDRISKKKKEYLLEE
RDINKFEVQKEFINRNLVDTRYATAELTNYLKAYFSANNMNVKVK
TINGSFTDYLRKVWKFKKERNHGYKHHAEDALIIANADFLFKENK
KLKAVNSVLEKPEIETKQLDIQVDSEDNYSEMFIIPKQVQDIKDF
RNFKYSHRVDKKPNRKLINDTLYSTRKKDNSTYIVQTIKDIYAKD
NTTLKKQFDKSPEKFLMYQHDPRTFEKLEVIMKQYANEKNPLAKY
HEETGEYLTKYSKKNNGPIVKSLKYIGNKLGSHLDVTHQFKSSTK
KLVKLSIKPYRFDVYLTDKGYKFITISYLDVLKKDNYYYIPEQKY
DKLKLGKAIDKNAKFIASFYKNDLIKLDGEIYKIIGVNSDTRNMI
ELDLPDIRYKEYCELNNIKGEPHIKKTIGKKVNSIEKLTTDVLGN
VFTNTQYTKPQLLFKRGN.

In some embodiments, the Cas protein is any of the engineered Cas proteins disclosed in Schmidt et al., 2021, Nature Communications, “Improved CRISPR genome editing using small highly active and specific engineered RNA-guided nucleases.”

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7021 (designated herein as sRGN1):

MNQKFILGLDIGITSVGYGLIDYETKNIIDAGVRLFPEANVENNE
GRRSKRGSRRLKRRRIHRLDRVKHLLAEYDLLDLTNIPKSTNPYQ
TRVKGLNEKLSKDELVIALLHIAKRRGIHNVDVAADKEETASDSL
STKDQINKNAKFLESRYVCELQKERLENEGHVRGVENRFLTKDIV
REAKKIIDTQMQYYPEIDETFKEKYISLVETRREYFEGPGKGSPF
GWEGNIKKWFEQMMGHCTYFPEELRSVKYSYSAELFNALNDLNNL
VITRDEDAKLNYGEKFQIIENVFKQKKTPNLKQIAIEIGVHETEI
KGYRVNKSGTPEFTEFKLYHDLKSIVFDKSILENEAILDQIAEIL
TIYQDEQSIKEELNKLPEILNEQDKAEIAKLIGYNGTHRLSLKCI
HLINEELWQTSRNQMEIFNYLNIKPNKVDLSEQNKIPKDMVNDFI
LSPVVKRTFIQSINVINKVIEKYGIPEDIIIELARENNSDDRKKF
INNLQKKNEATRKRINEIIGQTGNQNAKRIVEKIRLHDQQEGKCL
YSLKDIPLEDLLRNPNNYDIDHIIPRSVSFDDSMHNKVLVRREQN
AKKNNQTPYQYLTSGYADIKYSVFKQHVLNLAENKDRMTKKKREY
LLEERDINKFEVQKEFINRNLVDTRYATRELTNYLKAYFSANNMN
VKVKTINGSFTDYLRKVWKFKKERNHGYKHHAEDALIIANADFLF
KENKKLKAVNSVLEKPEIETKQLDIQVDSEDNYSEMFIIPKQVQD
IKDFRNFKYSHRVDKKPNRQLINDTLYSTRKKDNSTYIVQTIKDI
YAKDNTTLKKQFDKSPEKFLMYQHDPRTFEKLEVIMKQYANEKNP
LAKYHEETGEYLTKYSKKNNGPIVKSLKYIGNKLGSHLDVTHQFK
SSTKKLVKLSIKPYRFDVYLTDKGYKFITISYLDVLKKDNYYYIP
EQKYDKLKLGKAIDKNAKFIASFYKNDLIKLDGEIYKIIGVNSDT
RNMIELDLPDIRYKEYCELNNIKGEPRIKKTIGKKVNSIEKLTTD
VLGNVFTNTQYTKPQLLFKRGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7022 (designated herein as sRGN2):

MNQKFILGLDIGITSVGYGLIDYETKNIIDAGVRLFPEANVENNE
GRRSKRGSRRLKRRRIHRLERVKSLLSEYKIISGLAPTNNQPYNI
RVKGLTEQLTKDELAVALLHIAKRRGIHKIDVIDSNDDVGNELST
KEQLNKNSKLLKDKFVCQIQLERMNEGQVRGEKNRFKTADIIKEI
IQLLNVQKNFHQLDENFINKYIELVEMRREYFEGPGQGSPFGWNG
DLKKWYEMLMGHCTYFPQELRSVKYAYSADLFNALNDLNNLIIQR
DNSEKLEYHEKYHIIENVFKQKKKPTLKQIAKEIGVNPEDIKGYR
ITKSGTPEFTEFKLYHDLKSVLFDQSILENEDVLDQIAEILTIYQ
DKDSIKSKLTELDILLNEEDKENIAQLTGYNGTHRLSLKCIRLVL
EEQWYSSRNQMEIFTHLNIKPKKINLTAANKIPKAMIDEFILSPV
VKRTFIQSINVINKVIEKYGIPEDIIIELARENNSDDRKKFINNL
QKKNEATRKRINEIIGQTGNQNAKRIVEKIRLHDQQEGKCLYSLE
SIALMDLLNNPQNYEVDHIIPRSVAFDNSIHNKVLVKQIENSKKG
NRTPYQYLNSSDAKLSYNQFKQHILNLSKSKDRISKKKKDYLLEE
RDINKFEVQKEFINRNLVDTRYATRELTSYLKAYFSANNMDVKVK
TINGSFTNHLRKVWRFDKYRNHGYKHHAEDALIIANADFLFKENK
KLKAVNSVLEKPEIETKQLDIQVDSEDNYSEMFIIPKQVQDIKDF
RNFKYSHRVDKKPNRQLINDTLYSTRKKDNSTYIVQTIKDIYAKD
NTTLKKQFDKSPEKFLMYQHDPRTFEKLEVIMKQYANEKNPLAKY
HEETGEYLTKYSKKNNGPIVKSLKYIGNKLGSHLDVTHQFKSSTK
KLVKLSIKPYRFDVYLTDKGYKFITISYLDVLKKDNYYYIPEQKY
DKLKLGKAIDKNAKFIASFYKNDLIKLDGEIYKIIGVNSDTRNMI
ELDLPDIRYKEYCELNNIKGEPRIKKTIGKKVNSIEKLTTDVLGN
VFTNTQYTKPQLLFKRGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7023 (designated herein as sRGN3):

MNQKFILGLDIGITSVGYGLIDYETKNIIDAGVRLFPEANVENNEGRRSK
RGSRRLKRRRIHRLERVKLLLTEYDLINKEQIPTSNNPYQIRVKGLSEIL
SKDELAIALLHLAKRRGIHNVDVAADKEETASDSLSTKDQINKNAKFLES
RYVCELQKERLENEGHVRGVENRFLTKDIVREAKKIIDTQMQYYPEIDET
FKEKYISLVETRREYFEGPGQGSPFGWNGDLKKWYEMLMGHCTYFPQELR
SVKYAYSADLFNALNDLNNLIIQRDNSEKLEYHEKYHIIENVFKQKKKPT
LKQIAKEIGVNPEDIKGYRITKSGTPEFTSFKLFHDLKKVVKDHAILDDI
DLLNQIAEILTIYQDKDSIVAELGQLEYLMSEADKQSISELTGYTGTHSL
SLKCMNMIIDELWHSSMNQMEVFTYLNMRPKKYELKGYQRIPTDMIDDAI
LSPVVKRTFIQSINVINKVIEKYGIPEDIIIELARENNSDDRKKFINNLQ
KKNEATRKRINEIIGQTGNQNAKRIVEKIRLHDQQEGKCLYSLESIPLED
LLNNPNHYEVDHIIPRSVSFDNSYHNKVLVKQSENSKKSNLTPYQYFNSG
KSKLSYNQFKQHILNLSKSQDRISKKKKEYLLEERDINKFEVQKEFINRN
LVDTRYATRELTNYLKAYFSANNMNVKVKTINGSFTDYLRKVWKFKKERN
HGYKHHAEDALIIANADFLFKENKKLKAVNSVLEKPEIETKQLDIQVDSE
DNYSEMFIIPKQVQDIKDFRNFKYSHRVDKKPNRQLINDTLYSTRKKDNS
TYIVQTIKDIYAKDNTTLKKQFDKSPEKFLMYQHDPRTFEKLEVIMKQYA
NEKNPLAKYHEETGEYLTKYSKKNNGPIVKSLKYIGNKLGSHLDVTHQFK
SSTKKLVKLSIKPYRFDVYLTDKGYKFITISYLDVLKKDNYYYIPEQKYD
KLKLGKAIDKNAKFIASFYKNDLIKLDGEIYKIIGVNSDTRNMIELDLPD
IRYKEYCELNNIKGEPRIKKTIGKKVNSIEKLTTDVLGNVFTNTQYTKPQ
LLFKRGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7024 (designated herein as sRGN3.1):

MNQKFILGLDIGITSVGYGLIDYETKNIIDAGVRLFPEANVENNEGRRSK
RGSRRLKRRRIHRLERVKLLLTEYDLINKEQIPTSNNPYQIRVKGLSEIL
SKDELAIALLHLAKRRGIHNVDVAADKEETASDSLSTKDQINKNAKFLES
RYVCELQKERLENEGHVRGVENRFLTKDIVREAKKIIDTQMQYYPEIDET
FKEKYISLVETRREYFEGPGQGSPFGWNGDLKKWYEMLMGHCTYFPQELR
SVKYAYSADLFNALNDLNNLIIQRDNSEKLEYHEKYHIIENVFKQKKKPT
LKQIAKEIGVNPEDIKGYRITKSGTPEFTSFKLFHDLKKVVKDHAILDDI
DLLNQIAEILTIYQDKDSIVAELGQLEYLMSEADKQSISELTGYTGTHSL
SLKCMNMIIDELWHSSMNQMEVFTYLNMRPKKYELKGYQRIPTDMIDDAI
LSPVVKRTFIQSINVINKVIEKYGIPEDIIIELARENNSDDRKKFINNLQ
KKNEATRKRINEIIGQTGNQNAKRIVEKIRLHDQQEGKCLYSLESIPLED
LLNNPNHYEVDHIIPRSVSFDNSYHNKVLVKQSENSKKSNLTPYQYFNSG
KSKLSYNQFKQHILNLSKSQDRISKKKKEYLLEERDINKFEVQKEFINRN
LVDTRYATRELTNYLKAYFSANNMNVKVKTINGSFTDYLRKVWKFKKERN
HGYKHHAEDALIIANADFLFKENKKLKAVNSVLEKPEIETKQLDIQVDSE
DNYSEMFIIPKQVQDIKDFRNFKYSHRVDKKPNRQLINDTLYSTRKKDNS
TYIVQTIKDIYAKDNTTLKKQFDKSPEKFLMYQHDPRTFEKLEVIMKQYA
NEKNPLAKYHEETGEYLTKYSKKNNGPIVKSLKYIGNKLGSHLDVTHQFK
SSTKKLVKLSIKNYRFDVYLTEKGYKFVTIAYLNVFKKDNYYYIPKDKYQ
ELKEKKKIKDTDQFIASFYKNDLIKLNGDLYKIIGVNSDDRNIIELDYYD
IKYKDYCEINNIKGEPRIKKTIGKKTESIEKFTTDVLGNLYLHSTEKAPQ
LIFKRGL.

In some embodiments, the Cas9 comprises an amino acid sequence that is encoded by a nucleic acid molecule at least 80%, 85%, 90%, 91%, 92%, 93%, 94%, 95%, 96%, 97%, 98%, 99% or 100% identical to the sequence of SEQ ID NO: 918 (an exemplary nucleic acid molecule encoding sRGN3.1):

ATGAATCAGAAATTCATCCTGGGACTGGACATCGGCATTACCTCTGTGGG
CTACGGCTTGATTGACTACGAGACCAAGAACATCATCGACGCCGGCGTTA
GACTGTTTCCTGAGGCCAATGTGGAAAACAACGAGGGCAGACGGTCTAAG
CGGGGCTCTAGACGACTGAAAAGAAGAAGAATCCACAGACTGGAAAGAGT
GAAGCTGCTGCTGACCGAGTACGACCTGATCAATAAGGAACAGATCCCTA
CAAGCAACAACCCCTACCAGATTAGAGTGAAGGGCCTGAGCGAGATCCTG
AGCAAAGACGAGCTGGCCATCGCCCTGCTGCACCTGGCCAAGCGGAGGGG
CATCCACAACGTGGATGTGGCCGCCGACAAGGAGGAAACCGCCAGCGACT
CCCTGAGCACCAAAGATCAGATCAACAAGAATGCCAAGTTCCTGGAGAGC
AGATACGTGTGCGAGCTGCAGAAGGAGAGATTGGAGAACGAGGGCCACGT
GCGGGGCGTGGAGAATAGATTCCTGACAAAAGATATCGTGCGGGAAGCCA
AGAAGATCATCGACACCCAGATGCAGTACTATCCTGAAATCGACGAAACC
TTCAAGGAGAAGTACATCAGCCTGGTCGAAACCAGACGCGAGTATTTCGA
GGGCCCCGGACAGGGCTCTCCTTTCGGCTGGAACGGCGATCTGAAGAAGT
GGTACGAGATGCTGATGGGACACTGCACCTACTTCCCTCAAGAGCTGAGA
TCTGTGAAGTACGCCTACAGCGCCGATCTGTTCAACGCCCTGAACGATCT
GAACAACCTTATCATCCAGCGGGACAATTCTGAGAAGCTGGAATACCACG
AGAAATATCATATCATCGAGAACGTCTTTAAACAAAAGAAAAAGCCTACC
CTGAAGCAGATCGCCAAGGAGATCGGAGTGAATCCTGAGGATATCAAAGG
CTACAGAATCACAAAGTCCGGCACCCCTGAGTTCACCAGCTTTAAGCTGT
TCCACGACCTGAAGAAGGTCGTGAAAGACCACGCCATCCTCGATGACATC
GATCTGCTGAACCAGATCGCTGAGATCCTGACAATCTACCAGGACAAAGA
TTCTATCGTGGCTGAACTGGGACAGCTGGAATACCTTATGAGCGAGGCCG
ACAAGCAAAGCATCTCCGAACTGACCGGCTACACGGGCACCCATAGCCTG
AGCCTGAAGTGTATGAACATGATCATCGATGAGCTGTGGCACTCTAGCAT
GAACCAGATGGAAGTTTTCACCTACCTGAACATGCGGCCTAAGAAGTACG
AGCTGAAGGGCTACCAGAGAATCCCCACCGATATGATCGACGACGCCATC
CTGAGCCCCGTGGTGAAAAGAACATTCATCCAGAGCATCAACGTGATCAA
CAAGGTGATCGAGAAGTACGGCATTCCAGAGGACATCATCATCGAGCTGG
CCAGAGAAAACAACAGCGACGATAGAAAGAAGTTTATCAACAACCTGCAG
AAAAAAAACGAGGCCACCCGGAAGAGAATTAATGAGATCATCGGCCAGAC
AGGCAACCAGAACGCCAAAAGGATCGTGGAAAAAATCAGACTGCACGACC
AGCAGGAGGGCAAGTGCCTGTACTCTCTGGAAAGCATCCCCCTGGAGGAC
CTGCTGAACAATCCAAATCACTACGAGGTGGACCACATCATCCCTAGAAG
CGTCAGCTTCGACAACAGCTACCACAACAAGGTGCTGGTGAAGCAGAGCG
AAAACAGTAAGAAATCCAACCTGACACCTTACCAGTACTTTAACAGCGGC
AAGAGCAAGCTGAGCTACAACCAGTTCAAGCAGCACATCCTGAACCTGTC
CAAATCTCAGGATAGAATCTCCAAGAAAAAGAAGGAATACCTGCTGGAGG
AGAGAGATATCAACAAGTTCGAAGTCCAAAAGGAGTTCATCAACAGGAAC
CTGGTGGACACCCGGTACGCCACAAGAGAGCTGACAAACTACCTGAAAGC
CTACTTCAGCGCTAACAACATGAACGTGAAGGTGAAAACCATCAATGGAA
GTTTCACAGACTACCTTCGGAAGGTGTGGAAGTTCAAGAAGGAACGGAAT
CACGGCTACAAGCACCACGCAGAGGACGCCCTGATTATAGCTAATGCCGA
TTTCCTGTTCAAAGAAAACAAGAAGCTGAAAGCCGTGAACAGCGTTCTGG
AAAAGCCTGAAATTGAGACCAAGCAACTGGATATACAGGTGGACAGCGAG
GACAACTACTCCGAGATGTTCATCATCCCTAAACAGGTGCAGGACATCAA
AGACTTCAGAAATTTCAAGTACAGCCACAGAGTGGACAAGAAACCCAACC
GGCAGCTGATCAATGACACCCTGTATAGCACCCGCAAGAAGGATAACAGC
ACCTACATCGTCCAGACCATCAAGGACATCTACGCTAAGGACAACACCAC
CCTGAAAAAGCAATTTGATAAGTCCCCCGAAAAGTTTCTGATGTACCAAC
ACGATCCTAGAACCTTCGAAAAGTTGGAGGTGATCATGAAGCAATATGCC
AACGAGAAGAACCCACTGGCCAAGTACCATGAGGAGACAGGAGAATACCT
GACCAAGTATTCTAAGAAGAATAACGGCCCCATCGTGAAGTCCCTGAAGT
ACATTGGGAACAAACTCGGAAGCCACCTGGACGTGACGCACCAGTTCAAG
AGCAGCACCAAGAAGCTAGTGAAACTGAGCATCAAGAACTACAGATTCGA
CGTGTACCTGACAGAAAAGGGCTACAAATTTGTGACCATCGCTTACCTGA
ATGTGTTCAAAAAGGACAATTATTACTACATCCCAAAGGACAAGTACCAG
GAGCTTAAAGAGAAAAAAAAGATCAAGGATACCGACCAGTTTATCGCTAG
CTTCTACAAGAACGACCTGATTAAGCTGAACGGCGACCTGTACAAGATCA
TCGGCGTGAACTCTGACGACCGGAACATAATAGAGCTGGATTATTATGAC
ATCAAGTACAAGGACTACTGCGAGATCAACAACATCAAGGGCGAGCCTAG
AATCAAAAAGACCATCGGGAAGAAAACCGAGTCTATCGAAAAGTTTACAA
CAGACGTGCTGGGCAACCTGTACCTGCACAGCACGGAAAAGGCCCCTCAG
CTCATCTTCAAGAGAGGCCTG

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7025 (designated herein as sRGN3.2):

MNQKFILGLDIGITSVGYGLIDYETKNIIDAGVRLFPEANVENNEGRRSK
RGSRRLKRRRIHRLERVKLLLTEYDLINKEQIPTSNNPYQIRVKGLSEIL
SKDELAIALLHLAKRRGIHNVDVAADKEETASDSLSTKDQINKNAKFLES
RYVCELQKERLENEGHVRGVENRFLTKDIVREAKKIIDTQMQYYPEIDET
FKEKYISLVETRREYFEGPGQGSPFGWNGDLKKWYEMLMGHCTYFPQELR
SVKYAYSADLFNALNDLNNLIIQRDNSEKLEYHEKYHIIENVFKQKKKPT
LKQIAKEIGVNPEDIKGYRITKSGTPEFTSFKLFHDLKKVVKDHAILDDI
DLLNQIAEILTIYQDKDSIVAELGQLEYLMSEADKQSISELTGYTGTHSL
SLKCMNMIIDELWHSSMNQMEVFTYLNMRPKKYELKGYQRIPTDMIDDAI
LSPVVKRTFIQSINVINKVIEKYGIPEDIIIELARENNSDDRKKFINNLQ
KKNEATRKRINEIIGQTGNQNAKRIVEKIRLHDQQEGKCLYSLESIPLED
LLNNPNHYEVDHIIPRSVSFDNSYHNKVLVKQSENSKKSNLTPYQYFNSG
KSKLSYNQFKQHILNLSKSQDRISKKKKEYLLEERDINKFEVQKEFINRN
LVDTRYATRELTNYLKAYFSANNMNVKVKTINGSFTDYLRKVWKFKKERN
HGYKHHAEDALIIANADFLFKENKKLKAVNSVLEKPEIETKQLDIQVDSE
DNYSEMFIIPKQVQDIKDFRNFKFSHRVDKKPNRQLINDTLYSTRMKDEH
DYIVQTITDIYGKDNTNLKKQFNKNPEKFLMYQNDPKTFEKLSIIMKQYS
DEKNPLAKYYEETGEYLTKYSKKNNGPIVKKIKLLGNKVGNHLDVTNKYE
NSTKKLVKLSIKNYRFDVYLTEKGYKFVTIAYLNVFKKDNYYYIPKDKYQ
ELKEKKKIKDTDQFIASFYKNDLIKLNGDLYKIIGVNSDDRNIIELDYYD
IKYKDYCEINNIKGEPRIKKTIGKKTESIEKFTTDVLGNLYLHSTEKAPQ
LIFKRGL.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7026 (designated herein as sRGN3.3):

MNQKFILGLDIGITSVGYGLIDYETKNIIDAGVRLFPEANVENNEGRRSK
RGSRRLKRRRIHRLERVKLLLTEYDLINKEQIPTSNNPYQIRVKGLSEIL
SKDELAIALLHLAKRRGIHNVDVAADKEETASDSLSTKDQINKNAKFLES
RYVCELQKERLENEGHVRGVENRFLTKDIVREAKKIIDTQMQYYPEIDET
FKEKYISLVETRREYFEGPGQGSPFGWNGDLKKWYEMLMGHCTYFPQELR
SVKYAYSADLFNALNDLNNLIIQRDNSEKLEYHEKYHIIENVFKQKKKPT
LKQIAKEIGVNPEDIKGYRITKSGTPEFTSFKLFHDLKKVVKDHAILDDI
DLLNQIAEILTIYQDKDSIVAELGQLEYLMSEADKQSISELTGYTGTHSL
SLKCMNMIIDELWHSSMNQMEVFTYLNMRPKKYELKGYQRIPTDMIDDAI
LSPVVKRTFIQSINVINKVIEKYGIPEDIIIELARENNSDDRKKFINNLQ
KKNEATRKRINEIIGQTGNQNAKRIVEKIRLHDQQEGKCLYSLESIPLED
LLNNPNHYEVDHIIPRSVSFDNSYHNKVLVKQSENSKKSNLTPYQYFNSG
KSKLSYNQFKQHILNLSKSQDRISKKKKEYLLEERDINKFEVQKEFINRN
LVDTRYATRELTSYLKAYFSANNMDVKVKTINGSFTNHLRKVWRFDKYRN
HGYKHHAEDALIIANADFLFKENKKLQNTNKILEKPTIENNTKKVTVEKE
EDYNNVFETPKLVEDIKQYRDYKFSHRVDKKPNRQLINDTLYSTRMKDEH
DYIVQTITDIYGKDNTNLKKQFNKNPEKFLMYQNDPKTFEKLSIIMKQYS
DEKNPLAKYYEETGEYLTKYSKKNNGPIVKKIKLLGNKVGNHLDVTNKYE
NSTKKLVKLSIKNYRFDVYLTEKGYKFVTIAYLNVFKKDNYYYIPKDKYQ
ELKEKKKIKDTDQFIASFYKNDLIKLNGDLYKIIGVNSDDRNIIELDYYD
IKYKDYCEINNIKGEPRIKKTIGKKTESIEKFTTDVLGNLYLHSTEKAPQ
LIFKRGL.

In some embodiments, the Cas9 comprises an amino acid sequence that is encoded by a nucleic acid molecule at least 80%, 85%, 90%, 91%, 92%, 93%, 94%, 95%, 96%, 97%, 98%, 99% or 100% identical to the sequence of SEQ ID NO: 919 (an exemplary nucleic acid molecule encoding sRGN3.3):

AACCAGAAGTTTATCCTGGGCCTGGATATCGGCATCACATCCGTGGGCTA
CGGCCTGATTGATTACGAAACCAAGAACATCATTGATGCCGGCGTCCGGC
TTTTCCCTGAAGCTAACGTGGAAAACAATGAGGGCAGACGGAGCAAGAGA
GGCAGCAGACGGCTGAAGCGGAGAAGAATCCATAGACTCGAACGGGTGAA
GCTGCTGCTGACCGAGTACGACCTGATCAACAAGGAGCAGATCCCCACCA
GCAACAACCCATACCAGATCAGAGTGAAAGGCCTTTCTGAGATTCTGAGC
AAGGATGAGCTGGCTATCGCTCTGCTCCACCTGGCCAAGAGAAGGGGAAT
CCACAACGTTGACGTGGCCGCCGATAAGGAAGAGACCGCCAGCGATAGCC
TGAGCACCAAGGACCAGATCAACAAGAACGCCAAGTTCCTGGAAAGCAGA
TACGTGTGCGAGCTGCAAAAAGAGAGACTGGAAAATGAGGGCCATGTGCG
GGGCGTTGAGAACAGATTCCTGACCAAAGACATCGTCAGAGAGGCCAAGA
AAATTATTGACACCCAGATGCAGTACTATCCTGAGATAGACGAGACCTTT
AAGGAAAAGTACATCAGCCTGGTGGAAACAAGAAGAGAATACTTCGAAGG
ACCTGGCCAGGGCTCCCCTTTCGGCTGGAACGGCGACCTGAAGAAGTGGT
ACGAGATGCTGATGGGCCACTGCACCTACTTCCCCCAGGAGCTGCGGAGC
GTGAAGTACGCTTACAGCGCCGACCTGTTCAATGCCCTGAACGACCTGAA
CAATCTTATCATCCAAAGAGATAACAGCGAGAAATTAGAATACCACGAGA
AGTACCACATCATCGAGAATGTGTTCAAGCAAAAGAAGAAGCCTACCCTG
AAGCAGATCGCCAAAGAGATCGGCGTGAACCCTGAGGACATCAAGGGCTA
TCGGATCACCAAGTCCGGCACCCCTGAATTCACCAGCTTCAAGCTGTTTC
ACGACCTCAAAAAGGTCGTGAAGGACCACGCCATCCTGGACGACATCGAT
CTGCTGAATCAGATCGCCGAGATCCTGACCATCTACCAGGACAAGGACTC
TATCGTGGCCGAGCTTGGACAGCTGGAGTACCTGATGAGCGAGGCTGACA
AGCAGAGCATCAGCGAGCTGACCGGCTACACCGGAACCCACAGCCTGTCC
CTGAAGTGCATGAACATGATCATCGACGAGCTGTGGCACTCTAGCATGAA
CCAGATGGAAGTGTTCACCTACCTGAACATGAGACCTAAGAAGTACGAAC
TGAAGGGTTACCAGAGAATCCCAACCGACATGATCGACGACGCCATCCTG
AGCCCCGTGGTGAAGCGGACCTTTATCCAGAGCATCAATGTGATCAACAA
GGTGATCGAGAAATACGGCATCCCCGAGGACATCATCATCGAACTGGCCA
GAGAGAATAACTCTGATGACCGGAAGAAGTTCATCAACAACCTGCAGAAG
AAGAACGAAGCCACCAGAAAGCGCATCAACGAGATCATCGGCCAAACAGG
GAATCAGAACGCCAAGAGGATCGTGGAAAAGATTCGGCTGCACGACCAGC
AGGAGGGAAAATGCCTGTACAGCCTGGAAAGCATCCCCCTGGAAGATCTA
CTGAACAACCCCAACCACTACGAGGTGGATCACATCATCCCTAGAAGCGT
GTCCTTTGACAACAGTTACCACAACAAGGTGCTGGTGAAGCAATCCGAGA
ACTCGAAGAAGAGCAACCTGACACCTTACCAGTACTTTAACAGCGGCAAG
TCCAAGCTGTCTTATAACCAGTTCAAGCAGCACATCCTCAACCTGTCAAA
GTCTCAGGACAGAATCTCTAAGAAGAAGAAGGAGTATCTGCTGGAAGAGC
GGGACATCAACAAGTTCGAGGTGCAGAAAGAGTTCATTAACAGAAACCTG
GTCGACACCCGGTACGCCACACGCGAACTGACAAGCTACCTGAAGGCCTA
CTTCTCCGCCAACAATATGGACGTGAAGGTCAAGACCATCAATGGCAGCT
TCACAAATCACCTGAGAAAGGTCTGGCGGTTCGACAAGTACAGAAACCAC
GGCTACAAGCACCACGCCGAGGATGCTCTGATTATCGCCAACGCCGACTT
CCTGTTCAAGGAAAACAAAAAACTGCAGAACACCAACAAGATCCTGGAAA
AACCTACAATCGAGAACAACACAAAGAAAGTGACAGTGGAAAAAGAGGAA
GATTACAACAACGTGTTTGAGACACCTAAGCTGGTTGAGGATATCAAGCA
GTACCGGGACTATAAGTTTAGCCACAGAGTGGACAAGAAACCTAACAGGC
AGCTGATCAACGACACTCTGTACAGCACAAGAATGAAAGATGAGCACGAT
TACATCGTGCAGACCATTACCGACATCTACGGCAAGGACAACACCAATCT
GAAGAAGCAGTTCAACAAAAATCCCGAGAAGTTCCTGATGTACCAAAATG
ACCCTAAGACATTCGAGAAGCTGAGCATCATCATGAAACAGTACTCTGAT
GAGAAGAACCCACTGGCCAAGTACTACGAGGAAACAGGCGAATACCTGAC
CAAGTACTCTAAGAAGAACAACGGCCCTATCGTGAAGAAGATCAAACTGC
TGGGCAACAAAGTGGGAAATCATCTGGATGTGACCAATAAATACGAGAAC
TCTACAAAGAAACTGGTGAAGCTGAGCATTAAGAACTACAGATTCGACGT
CTACCTGACAGAAAAGGGATACAAGTTCGTGACCATCGCCTACCTGAACG
TGTTCAAGAAAGACAACTACTACTACATCCCAAAAGACAAGTACCAAGAG
TTAAAAGAGAAGAAGAAGATAAAGGATACCGACCAGTTTATCGCTTCTTT
CTACAAGAACGACCTGATCAAGCTGAACGGTGATCTGTACAAAATCATCG
GAGTGAATAGCGATGACAGAAATATCATCGAACTGGATTACTATGACATC
AAGTACAAAGATTATTGTGAAATCAACAACATCAAGGGAGAGCCCAGAAT
TAAGAAAACCATCGGCAAGAAAACAGAGAGCATCGAGAAATTCACCACAG
ATGTGCTGGGCAACCTGTACCTGCACAGCACAGAGAAAGCCCCTCAGCTC
ATCTTCAAGAGAGGCCTG

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7027 (designated herein as sRGN4):

MNQKFILGLDIGITSVGYGLIDYETKNIIDAGVRLFPEANVENNEGRRSK
RGSRRLKRRRIHRLERVKKLLEDYNLLDQSQIPQSTNPYAIRVKGLSEAL
SKDELVIALLHIAKRRGIHNINVSSEDEDASNELSTKEQINRNNKLLKDK
YVCEVQLQRLKEGQIRGEKNRFKTTDILKEIDQLLKVQKDYHNLDIDFIN
QYKEIVETRREYFEGPGKGSPYGWEGDPKAWYETLMGHCTYFPDELRSVK
YAYSADLFNALNDLNNLVIQRDGLSKLEYHEKYHIIENVFKQKKKPTLKQ
IANEINVNPEDIKGYRITKSGKPEFTSFKLFHDLKKVVKDHAILDDIDLL
NQIAEILTIYQDKDSIVAELGQLEYLMSEADKQSISELTGYTGTHSLSLK
CMNMIIDELWHSSMNQMEVFTYLNMRPKKYELKGYQRIPTDMIDDAILSP
VVKRTFIQSINVINKVIEKYGIPEDIIIELARENNSDDRKKFINNLQKKN
EATRKRINEIIGQTGNQNAKRIVEKIRLHDQQEGKCLYSLESIPLEDLLN
NPNHYEVDHIIPRSVSFDNSYHNKVLVKQSENSKKSNLTPYQYFNSGKSK
LSYNQFKQHILNLSKSQDRISKKKKEYLLEERDINKFEVQKEFINRNLVD
TRYATRELTNYLKAYFSANNMNVKVKTINGSFTDYLRKVWKFKKERNHGY
KHHAEDALIIANADFLFKENKKLKAVNSVLEKPEIETKQLDIQVDSEDNY
SEMFIIPKQVQDIKDFRNFKYSHRVDKKPNRQLINDTLYSTRKKDNSTYI
VQTIKDIYAKDNTTLKKQFDKSPEKFLMYQHDPRTFEKLEVIMKQYANEK
NPLAKYHEETGEYLTKYSKKNNGPIVKSLKYIGNKLGSHLDVTHQFKSST
KKLVKLSIKPYRFDVYLTDKGYKFITISYLDVLKKDNYYYIPEQKYDKLK
LGKAIDKNAKFIASFYKNDLIKLDGEIYKIIGVNSDTRNMIELDLPDIRY
KEYCELNNIKGEPRIKKTIGKKVNSIEKLTTDVLGNVFTNTQYTKPQLLF
KRGN.

In some embodiments, the Cas9 comprises an amino acid sequence that is encoded by a nucleic acid molecule at least 80%, 85%, 90%, 91%, 92%, 93%, 94%, 95%, 96%, 97%, 98%, 99% or 100% identical to the sequence of SEQ ID NO: 920 (an exemplary nucleic acid molecule encoding sRGN4):

ATGAATCAAAAGTTTATCCTGGGTCTGGACATCGGCATCACATCTGTGGG
CTACGGCCTTATCGATTACGAGACCAAGAATATCATTGATGCTGGCGTAC
GGCTGTTCCCTGAGGCTAACGTGGAAAACAACGAGGGTAGACGGAGCAAG
AGAGGCAGCAGACGGCTGAAACGGCGTAGAATCCACCGGCTGGAGAGAGT
GAAGAAGTTGCTGGAAGATTACAACTTGCTGGATCAATCCCAGATCCCCC
AGAGCACTAATCCTTATGCTATCCGGGTGAAGGGCCTGTCTGAAGCCCTG
AGCAAAGACGAGCTGGTGATTGCCCTGCTGCACATCGCGAAGAGAAGAGG
CATCCACAATATCAATGTGTCCTCTGAGGATGAGGATGCCAGCAACGAGC
TGAGCACTAAGGAACAGATCAATCGGAACAACAAGCTGCTGAAGGACAAG
TACGTGTGTGAAGTGCAGCTGCAGAGACTGAAGGAAGGCCAGATAAGAGG
CGAAAAGAACAGATTCAAGACAACAGACATCCTGAAAGAAATCGACCAGC
TGCTGAAGGTCCAGAAGGACTACCACAACCTCGACATCGATTTCATTAAC
CAGTACAAGGAAATCGTGGAAACCAGGAGAGAGTACTTCGAGGGCCCTGG
CAAGGGCTCCCCATACGGCTGGGAGGGCGACCCCAAGGCCTGGTACGAAA
CCCTGATGGGCCACTGCACCTACTTCCCCGACGAACTGAGAAGCGTCAAG
TACGCCTACAGCGCCGATCTGTTCAACGCCCTGAACGACCTGAACAACCT
GGTGATCCAGCGGGACGGCCTGAGCAAACTGGAGTACCATGAAAAGTATC
ATATCATCGAGAACGTGTTCAAGCAGAAGAAAAAACCTACCCTGAAGCAG
ATCGCCAACGAGATCAACGTGAACCCTGAGGACATTAAAGGCTACAGAAT
CACCAAAAGCGGCAAGCCAGAGTTCACCAGCTTCAAGCTGTTTCACGACC
TGAAGAAGGTCGTGAAAGACCACGCCATCCTGGACGACATCGATCTGCTT
AACCAGATCGCTGAAATCCTCACAATCTACCAGGACAAGGACTCTATCGT
GGCCGAGCTGGGACAGCTGGAATACCTGATGAGCGAGGCCGATAAGCAGA
GCATCAGCGAGCTGACCGGCTACACCGGAACCCACAGCCTGAGCCTGAAG
TGTATGAACATGATCATCGACGAGCTGTGGCACAGCTCTATGAACCAGAT
GGAAGTATTCACCTACCTGAACATGAGACCTAAGAAGTACGAACTGAAGG
GCTATCAGAGAATCCCTACAGACATGATCGACGATGCCATCCTGTCTCCT
GTGGTGAAGAGAACCTTCATCCAGTCTATCAACGTGATCAACAAGGTGAT
CGAAAAGTACGGAATCCCTGAAGATATCATCATCGAACTGGCCAGAGAGA
ACAACTCCGACGACAGAAAGAAATTCATCAACAACCTGCAGAAGAAGAAT
GAGGCCACACGGAAGCGGATTAATGAGATCATCGGCCAAACCGGCAACCA
GAACGCCAAAAGAATCGTGGAAAAGATCCGGCTGCACGATCAGCAGGAGG
GCAAATGCCTGTACAGCCTGGAGAGCATCCCCCTGGAGGACCTGCTCAAC
AACCCCAACCACTACGAGGTGGATCACATCATCCCAAGATCTGTTAGCTT
CGACAACAGCTACCACAACAAGGTGCTGGTGAAGCAAAGCGAAAACTCTA
AGAAATCTAACCTGACACCTTACCAGTACTTTAACAGCGGCAAGTCCAAG
CTGTCTTATAACCAGTTTAAGCAGCACATCCTGAACCTGAGCAAGTCCCA
GGATAGAATCAGCAAAAAAAAGAAGGAATACCTGCTGGAGGAACGCGACA
TCAATAAATTTGAGGTGCAAAAGGAGTTCATCAACCGGAACCTGGTGGAC
ACCCGGTACGCCACCAGAGAACTGACCAACTACCTGAAAGCCTACTTCAG
CGCCAACAACATGAACGTGAAGGTGAAAACCATCAACGGAAGCTTCACCG
ACTACCTGCGGAAGGTGTGGAAGTTCAAGAAAGAGCGGAATCACGGCTAT
AAGCACCACGCTGAGGACGCTCTGATCATCGCCAATGCCGATTTCCTGTT
CAAGGAAAACAAGAAGTTAAAGGCCGTGAACTCTGTCCTGGAGAAGCCCG
AGATCGAGACAAAGCAGCTGGACATCCAAGTGGACTCAGAGGACAATTAC
TCTGAGATGTTCATCATCCCCAAGCAGGTGCAGGACATCAAGGATTTTAG
AAATTTCAAGTACAGCCATAGAGTGGACAAGAAGCCCAATAGACAGCTGA
TCAACGATACACTGTACAGCACCAGAAAGAAGGACAACAGCACATATATC
GTCCAGACCATCAAGGACATTTACGCAAAGGATAACACCACACTGAAGAA
GCAGTTCGACAAAAGCCCTGAGAAGTTCCTGATGTACCAACACGACCCTC
GGACCTTCGAGAAGTTAGAGGTTATCATGAAACAGTACGCCAACGAGAAA
AACCCTCTGGCCAAGTACCACGAGGAAACCGGAGAATACCTGACAAAATA
TAGCAAGAAGAACAACGGCCCCATCGTGAAAAGCCTGAAGTACATCGGCA
ACAAGCTGGGCAGCCACCTGGACGTGACCCACCAGTTCAAGAGCAGCACC
AAGAAGCTGGTCAAGCTGAGCATCAAGCCTTATAGGTTCGACGTGTACCT
GACAGATAAGGGATACAAGTTCATCACCATCAGCTACCTGGATGTTCTGA
AAAAGGACAATTACTACTACATCCCTGAGCAGAAGTACGACAAACTCAAG
CTGGGCAAGGCCATCGATAAGAATGCCAAGTTCATCGCATCTTTTTACAA
GAACGACCTGATCAAACTGGACGGCGAGATCTACAAGATCATAGGAGTGA
ACAGCGACACCAGGAATATGATCGAGCTCGATCTGCCTGACATCAGATAC
AAGGAATACTGCGAGCTGAACAACATCAAGGGAGAGCCTAGAATCAAGAA
AACCATCGGCAAGAAGGTGAACAGCATCGAGAAACTTACAACAGATGTGC
TCGGCAACGTGTTCACCAACACCCAGTACACCAAGCCACAGCTGCTGTTT
AAGCGGGGGAAC

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7028 (designated herein as Staphylococcus hyicus Cas9 or ShyCas9):

MNNYILGLDIGITSVGYGIVDSDTREIKDAGVRLFPEANVDNNEGRRSKR
GARRLKRRRIHRLDRVKHLLAEYDLLDLTNIPKSTNPYQTRVKGLNEKLS
KDELVIALLHIAKRRGIHNVNVMMDDNDSGNELSTKDQLKKNAKALSDKY
VCELQLERFEQDYKVRGEKNRFKTEDFVREARKLLETQSKFFEIDQTFIM
RYIELIETRREYFEGPGKGSPFGWEGNIKKWFEQMMGHCTYFPEELRSVK
YSYSAELFNALNDLNNLVITRDEDAKLNYGEKFQIIENVFKQKKTPNLKQ
IAIEIGVHETEIKGYRVNKSGKPEFTQFKLYHDLKNIFKDPKYLNDIQLM
DNIAEIITIYQDAESIIKELNQLPELLSEREKEKISALSGYSGTHRLSLK
CINLLLDDLWESSLNQMELFTKLNLKPKKIDLSQQHKIPSKLVDDFILSP
VVKRAFIQSIQVVNAIIDKYGLPEDIIIELARENNSDDRRKFLNQLQKQN
EETRKQVEKVLREYGNDNAKRIVQKIKLHNMQEGKCLYSLKDIPLEDLLR
NPHHYEVDHIIPRSVAFDNSMHNKVLVRADENSKKGNRTPYQYLNSSESS
LSYNEFKQHILNLSKTKDRITKKKREYLLEERDINKFDVQKEFINRNLVD
TRYATRELTSLLKAYFSANNLDVKVKTINGSFTNYLRKVWKFDKDRNKGY
KHHAEDALIIANADFLFKHNKKLRNINKVLDAPSKEVDKKRVTVQSEDEY
NQIFEDTQKAQAIKKFEIRKFSHRVDKKPNRQLINDTLYSTRNIDGIEYV
VESIKDIYSVNNDKVKTKFKKDPHRLLMYRNDPQTFEKFEKVFKQYESEK
NPFAKYYEETGEKIRKFSKTGQGPYINKIKYLRERLGRHCDVTNKYINSR
NKIVQLKIYSYRFDIYQYGNNYKMITISYIDLEQKSNYYYISREKYEQKK
KDKQIDDSYKFIGSFYKNDIINYNGEMYRVIGVNDSEKNKIQLDMIDISI
KDYMELNNIKKTGVIYKTIGKSTTHIEKYTTDILGNLYKAAPPKKPQLIF
K.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7029 (designated herein as Staphylococcus microti Cas9 or Smi Cas9):

MEKDYILGLDIGIGSVGYGLIDYDTKSIIDAGVRLFPEANADNNLGRRAK
RGARRLKRRRIHRLERVKSLLSEYKIISGLAPTNNQPYNIRVKGLTEQLT
KDELAVALLHIAKRRGIHNVDVAADKEETASDSLSTKDQINKNAKFLESR
YVCELQKERLENEGHVRGVENRFLTKDIVREAKKIIDTQMQYYPEIDETF
KEKYISLVETRREYYEGPGKGSPYGWDADVKKWYQLMMGHCTYFPVEFRS
VKYAYTADLYNALNDLNNLTIARDDNPKLEYHEKYHIIENVFKQKRNPTL
KQIAKEIGVNDINISGYRVTKSGKPQFTSFKLFHDLKKVVKDHAILDDID
LLNQIAEILTIYQDKDSIVAELGQLEYLMSEADKQSISELTGYTGTHSLS
LKCMNMIIDELWHSSMNQMEVFTYLNMRPKKYELKGYQRIPTDMIDDAIL
SPVVKRSFKQAIGVVNAIIKKYGLPKDIIIELARESNSAEKSRYLRAIQK
KNEKTRERIEAIIKEYGNENAKGLVQKIKLHDAQEGKCLYSLKDIPLEDL
LRNPNNYDIDHIIPRSVSFDDSMHNKVLVRREQNAKKNNQTPYQYLTSGY
ADIKYSVFKQHVLNLAENKDRMTKKKREYLLEERNINKYDVQKEFINRNL
VDTRYTTRELTTLLKTYFTINNLDVKVKTINGSFTDFLRKRWGFKKNRDE
GYKHHAEDALIIANADYLFKEHKLLKEIKDVSDLAGDERNSNVKDEDQYE
EVFGGYFKIEDIKKYKIKKFSHRVDKKPNRQLINDTIYSTRVKDDKRYLI
NTLKNLYDKSNGDLKERMQKDPESLLMYHHDPQTFEKLKIVMSQYENEKN
PLAKYFEETGQYLTKYAKHDNGPAIHKIKYYGNKLVEHLDITKNYHNPQN
KVVQLSQKSFRFDVYQTDKGYKFISIAYLTLKNEKNYYAISQEKYDQLKS
EKKISNNAVFIGSFYTSDIIEINNEKFRVIGVNSDKNNLIEVDRIDIRQK
EFIELEEEKKNNRIKVTIGRKTTNIEKFHTDILGNMYKSKRPKAPQLVFK
KG.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7030 (designated herein as Staphylococcus pasteuri Cas9 or Spa Cas9):

MKEKYILGLDLGITSVGYGIINFETKKIIDAGVRLFPEANVDNNEGRRSK
RGSRRLKRRRIHRLERVKLLLTEYDLINKEQIPTSNNPYQIRVKGLSEIL
SKDELAIALLHLAKRRGIHNINVSSEDEDASNELSTKEQINRNNKLLKDK
YVCEVQLQRLKEGQIRGEKNRFKTTDILKEIDQLLKVQKDYHNLDIDFIN
QYKEIVETRREYFEGPGQGSPFGWNGDLKKWYEMLMGHCTYFPQELRSVK
YAYSADLFNALNDLNNLIIQRDNSEKLEYHEKYHIIENVFKQKKKPTLKQ
IAKEIGVNPEDIKGYRITKSGTPQFTEFKLYHDLKSIVFDKSILENEAIL
DQIAEILTIYQDEQSIKEELNKLPEILNEQDKAEIAKLIGYNGTHRLSLK
CIHLINEELWQTSRNQMEIFNYLNIKPNKVDLSEQNKIPKDMVNDFILSP
VVKRTFIQSINVINKVIEKYGIPEDIIIELARENNSDDRKKFINNLQKKN
EATRKRINEIIGQTGNQNAKRIVEKIRLHDQQEGKCLYSLESIALMDLLN
NPQNYEVDHIIPRSVAFDNSIHNKVLVKQIENSKKGNRTPYQYLNSSDAK
LSYNQFKQHILNLSKSKDRISKKKKDYLLEERDINKFEVQKEFINRNLVD
TRYATRELTSYLKAYFSANNMDVKVKTINGSFTNHLRKVWRFDKYRNHGY
KHHAEDALIIANADFLFKENKKLQNTNKILEKPTIENNTKKVTVEKEEDY
NNVFETPKLVEDIKQYRDYKFSHRVDKKPNRQLINDTLYSTRMKDEHDYI
VQTITDIYGKDNTNLKKQFNKNPEKFLMYQNDPKTFEKLSIIMKQYSDEK
NPLAKYYEETGEYLTKYSKKNNGPIVKKIKLLGNKVGNHLDVTNKYENST
KKLVKLSIKNYRFDVYLTEKGYKFVTIAYLNVFKKDNYYYIPKDKYQELK
EKKKIKDTDQFIASFYKNDLIKLNGDLYKIIGVNSDDRNIIELDYYDIKY
KDYCEINNIKGEPRIKKTIGKKTESIEKFTTDVLGNLYLHSTEKAPQLIF
KRGL.

In some embodiments, the Cas protein comprises an amino acid sequence that is at least 80%, 85%, 90%, 91%, 92%, 93%, 94%, 95%, 96%, 97%, 98%, 99% or 100% identical to the sequence of SEQ ID NO: 7031 (designated herein as Cas12i1):

MSNKEKNASETRKAYTTKMIPRSHDRMKLLGNFMDYLMDGTPIFFELWNQ
FGGGIDRDIISGTANKDKISDDLLLAVNWFKVMPINSKPQGVSPSNLANL
FQQYSGSEPDIQAQEYFASNFDTEKHQWKDMRVEYERLLAELQLSRSDMH
HDLKLMYKEKCIGLSLSTAHYITSVMFGTGAKNNRQTKHQFYSKVIQLLE
ESTQINSVEQLASIILKAGDCDSYRKLRIRCSRKGATPSILKIVQDYELG
TNHDDEVNVPSLIANLKEKLGRFEYECEWKCMEKIKAFLASKVGPYYLGS
YSAMLENALSPIKGMTTKNCKFVLKQIDAKNDIKYENEPFGKIVEGFFDS
PYFESDTNVKWVLHPHHIGESNIKTLWEDLNAIHSKYEEDIASLSEDKKE
KRIKVYQGDVCQTINTYCEEVGKEAKTPLVQLLRYLYSRKDDIAVDKIID
GITFLSKKHKVEKQKINPVIQKYPSFNFGNNSKLLGKIISPKDKLKHNLK
CNRNQVDNYIWIEIKVLNTKTMRWEKHHYALSSTRFLEEVYYPATSENPP
DALAARFRTKTNGYEGKPALSAEQIEQIRSAPVGLRKVKKRQMRLEAARQ
QNLLPRYTWGKDFNINICKRGNNFEVTLATKVKKKKEKNYKVVLGYDANI
VRKNTYAAIEAHANGDGVIDYNDLPVKPIESGFVTVESQVRDKSYDQLSY
NGVKLLYCKPHVESRRSFLEKYRNGTMKDNRGNNIQIDFMKDFEAIADDE
TSLYYFNMKYCKLLQSSIRNHSSQAKEYREEIFELLRDGKLSVLKLSSLS
NLSFVMFKVAKSLIGTYFGHLLKKPKNSKSDVKAPPITDEDKQKADPEMF
ALRLALEEKRLNKVKSKKEVIANKIVAKALELRDKYGPVLIKGENISDTT
KKGKKSSTNSFLMDWLARGVANKVKEMVMMHQGLEFVEVNPNFTSHQDPF
VHKNPENTFRARYSRCTPSELTEKNRKEILSFLSDKPSKRPTNAYYNEGA
MAFLATYGLKKNDVLGVSLEKFKQIMANILHQRSEDQLLFPSRGGMFYLA
TYKLDADATSVNWNGKQFWVCNADLVAAYNVGLVDIQKDFKKK.

In some embodiments, the Cas protein comprises an amino acid sequence that is at least 80%, 85%, 90%, 91%, 92%, 93%, 94%, 95%, 96%, 97%, 98%, 99% or 100% identical to the sequence of SEQ ID NO: 7032 (designated herein as Cas12i2):

MSSAIKSYKSVLRPNERKNQLLKSTIQCLEDGSAFFFKMLQGLFGGITPE
IVRFSTEQEKQQQDIALWCAVNWFRPVSQDSLTHTIASDNLVEKFEEYYG
GTASDAIKQYFSASIGESYYWNDCRQQYYDLCRELGVEVSDLTHDLEILC
REKCLAVATESNQNNSIISVLFGTGEKEDRSVKLRITKKILEAISNLKEI
PKNVAPIQEIILNVAKATKETFRQVYAGNLGAPSTLEKFIAKDGQKEFDL
KKLQTDLKKVIRGKSKERDWCCQEELRSYVEQNTIQYDLWAWGEMFNKAH
TALKIKSTRNYNFAKQRLEQFKEIQSLNNLLVVKKLNDFFDSEFFSGEET
YTICVHHLGGKDLSKLYKAWEDDPADPENAIVVLCDDLKNNFKKEPIRNI
LRYIFTIRQECSAQDILAAAKYNQQLDRYKSQKANPSVLGNQGFTWTNAV
ILPEKAQRNDRPNSLDLRIWLYLKLRHPDGRWKKHHIPFYDTRFFQEIYA
AGNSPVDTCQFRTPRFGYHLPKLTDQTAIRVNKKHVKAAKTEARIRLAIQ
QGTLPVSNLKITEISATINSKGQVRIPVKFDVGRQKGTLQIGDRFCGYDQ
NQTASHAYSLWEVVKEGQYHKELGCFVRFISSGDIVSITENRGNQFDQLS
YEGLAYPQYADWRKKASKFVSLWQITKKNKKKEIVTVEAKEKFDAICKYQ
PRLYKFNKEYAYLLRDIVRGKSLVELQQIRQEIFRFIEQDCGVTRLGSLS
LSTLETVKAVKGIIYSYFSTALNASKNNPISDEQRKEFDPELFALLEKLE
LIRTRKKKQKVERIANSLIQTCLENNIKFIRGEGDLSTTNNATKKKANSR
SMDWLARGVFNKIRQLAPMHNITLFGCGSLYTSHQDPLVHRNPDKAMKCR
WAAIPVKDIGDWVLRKLSQNLRAKNIGTGEYYHQGVKEFLSHYELQDLEE
ELLKWRSDRKSNIPCWVLQNRLAEKLGNKEAVVYIPVRGGRIYFATHKVA
TGAVSIVFDQKQVWVCNADHVAAANIALTVKGIGEQSSDEENPDGSRIKL
QLTS.

### Modified Guide RNAs

In some embodiments, the guide RNA is chemically modified. A guide RNA comprising one or more modified nucleosides or nucleotides is called a “modified” guide RNA or “chemically modified” guide RNA, to describe the presence of one or more non-naturally and/or naturally occurring components or configurations that are used instead of or in addition to the canonical A, G, C, and U residues. In some embodiments, a modified guide RNA is synthesized with a non-canonical nucleoside or nucleotide, is here called “modified.” Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification).

Chemical modifications such as those listed above can be combined to provide modified guide RNAs comprising nucleosides and nucleotides (collectively “residues”) that can have two, three, four, or more modifications. For example, a modified residue can have a modified sugar and a modified nucleobase, or a modified sugar and a modified phosphodiester. In some embodiments, every base of a guide RNA is modified, e.g., all bases have a modified phosphate group, such as a phosphorothioate group. In certain embodiments, all, or substantially all, of the phosphate groups of a guide RNA molecule are replaced with phosphorothioate groups. In some embodiments, modified guide RNAs comprise at least one modified residue at or near the 5′ end of the RNA. In some embodiments, modified guide RNAs comprise at least one modified residue at or near the 3′ end of the RNA.

In some embodiments, the guide RNA comprises one, two, three or more modified residues. In some embodiments, at least 5% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) of the positions in a modified guide RNA are modified nucleosides or nucleotides.

Unmodified nucleic acids can be prone to degradation by, e.g., intracellular nucleases or those found in serum. For example, nucleases can hydrolyze nucleic acid phosphodiester bonds. Accordingly, in one aspect the guide RNAs described herein can contain one or more modified nucleosides or nucleotides, e.g., to introduce stability toward intracellular or serum-based nucleases. In some embodiments, the modified guide RNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group, i.e. at sugar modification. For example, the 2′ hydroxyl group (OH) can be modified, e.g. replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the 2′ hydroxyl group modification can include “unlocked” nucleic acids (UNA) in which the ribose ring lacks the C2′-C3′ bond. In some embodiments, the 2′ hydroxyl group modification can include the methoxyethyl group (MOE), (OCH₂CH₂OCH₃, e.g., a PEG derivative).

“Deoxy” 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH2CH₂— amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g. L-nucleosides.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA and/or tracr RNA. In embodiments comprising sgRNA, one or more residues at one or both ends of the sgRNA may be chemically modified, and/or internal nucleosides may be modified, and/or the entire sgRNA may be chemically modified. Certain embodiments comprise a 5′ end modification. Certain embodiments comprise a 3′ end modification.

Modifications of 2′-O-methyl are encompassed.

Another chemical modification that has been shown to influence nucleotide sugar rings is halogen substitution. For example, 2′-fluoro (2′-F) substitution on nucleotide sugar rings can increase oligonucleotide binding affinity and nuclease stability. Modifications of 2′-fluoro (2′-F) are encompassed.

Phosphorothioate (PS) linkage or bond refers to a bond where a sulfur is substituted for one nonbridging phosphate oxygen in a phosphodiester linkage, for example in the bonds between nucleotides bases. When phosphorothioates are used to generate oligonucleotides, the modified oligonucleotides may also be referred to as S-oligos.

Abasic nucleotides refer to those which lack nitrogenous bases.

Inverted bases refer to those with linkages that are inverted from the normal 5′ to 3′ linkage (i.e., either a 5′ to 5′ linkage or a 3′ to 3′ linkage).

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In some embodiments, one or more of the first three, four, or five nucleotides at the 5′ terminus, and one or more of the last three, four, or five nucleotides at the 3′ terminus are modified. In some embodiments, the modification is a 2′-O-Me, 2′-F, inverted abasic nucleotide, PS bond, or other nucleotide modification well known in the art to increase stability and/or performance.

In some embodiments, the first four nucleotides at the 5′ terminus, and the last four nucleotides at the 3′ terminus are linked with phosphorothioate (PS) bonds.

In some embodiments, the first three nucleotides at the 5′ terminus, and the last three nucleotides at the 3′ terminus comprise a 2′-O-methyl (2′-O-Me) modified nucleotide. In some embodiments, the first three nucleotides at the 5′ terminus, and the last three nucleotides at the 3′ terminus comprise a 2′-fluoro (2′-F) modified nucleotide.

### Ribonucleoprotein Complex

In some embodiments, a composition is encompassed comprising: a) one or more guide RNAs comprising one or more guide sequences from Table 6 and b) SaCas9 (when combined with a gRNA comprising any one of or combination of SEQ ID Nos: 1-159) or SluCas9 (when combined with a gRNA comprising any one of or combination of SEQ ID Nos: 200-292, 924-938, or 950-955), or any of the mutant Cas9 proteins disclosed herein. In some embodiments, the guide RNA together with a Cas9 is called a ribonucleoprotein complex (RNP).

In some embodiments, the disclosure provides for an RNP complex, wherein the guide RNA (e.g., any of the guide RNAs disclosed herein) binds to or is capable of binding to a target sequence in the dystrophin gene.

In some embodiments, chimeric Cas9 (SaCas9 or SluCas9) nucleases are used, where one domain or region of the protein is replaced by a portion of a different protein. In some embodiments, a Cas9 nuclease domain may be replaced with a domain from a different nuclease such as Fok1. In some embodiments, a Cas9 nuclease may be a modified nuclease.

In some embodiments, the Cas9 is modified to contain only one functional nuclease domain. For example, the agent protein may be modified such that one of the nuclease domains is mutated or fully or partially deleted to reduce its nucleic acid cleavage activity.

In some embodiments, a conserved amino acid within a Cas9 protein nuclease domain is substituted to reduce or alter nuclease activity. In some embodiments, a Cas9 nuclease may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell October 22:163(3): 759-771. In some embodiments, the Cas9 nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein). See, e.g., Zetsche et al. (2015). Further exemplary amino acid substitutions include D917A, E1006A, and D1255A (based on the Francisella novicida U112 Cpf1 (FnCpf1) sequence (UniProtKB-AOQ7Q2 (CPF1_FRATN)). Further exemplary amino acid substitutions include D10A and N580A (based on the S. aureus Cas9 protein). See, e.g., Friedland et al., 2015, Genome Biol., 16:257.

In some embodiments, the Cas9 lacks cleavase activity. In some embodiments, the Cas9 comprises a dCas DNA-binding polypeptide. A dCas polypeptide has DNA-binding activity while essentially lacking catalytic (cleavase/nickase) activity. In some embodiments, the dCas polypeptide is a dCas9 polypeptide. In some embodiments, the Cas9 lacking cleavase activity or the dCas DNA-binding polypeptide is a version of a Cas nuclease (e.g., a Cas9 nuclease discussed above) in which its endonucleolytic active sites are inactivated, e.g., by one or more alterations (e.g., point mutations) in its catalytic domains. See, e.g., US 2014/0186958 A1; US 2015/0166980 A1.

In some embodiments, the Cas9 comprises one or more heterologous functional domains (e.g., is or comprises a fusion polypeptide).

In some embodiments, the heterologous functional domain may facilitate transport of the Cas9 into the nucleus of a cell. For example, the heterologous functional domain may be a nuclear localization signal (NLS). In some embodiments, the Cas9 may be fused with 1-10 NLS(s). In some embodiments, the Cas9 may be fused with 1-5 NLS(s). In some embodiments, the Cas9 may be fused with 1-3 NLS(s). In some embodiments, the Cas9 may be fused with one NLS. Where one NLS is used, the NLS may be attached at the N-terminus or the C-terminus of the Cas9 sequence, and may be directly fused/attached. In some embodiments, where more than one NLS is used, one or more NLS may be attached at the N-terminus and/or one or more NLS may be attached at the C-terminus. In some embodiments, one or more NLSs are directly attached to the Cas9. In some embodiments, one or more NLSs are attached to the Cas9 by means of a linker. In some embodiments, the linker is between 3-25 amino acids in length. In some embodiments, the linker is between 3-6 amino acids in length. In some embodiments, the linker comprises glycine and serine. In some embodiments, the linker comprises the sequence of GSVD (SEQ ID NO: 550) or GSGS (SEQ ID NO: 551). It may also be inserted within the Cas9 sequence. In other embodiments, the Cas9 may be fused with more than one NLS. In some embodiments, the Cas9 may be fused with 2, 3, 4, or 5 NLSs. In some embodiments, the Cas9 may be fused with two NLSs. In certain circumstances, the two NLSs may be the same (e.g., two SV40 NLSs) or different. In some embodiments, the Cas9 protein is fused with one or more SV40 NLSs. In some embodiments, the SV40 NLS comprises the amino acid sequence of SEQ ID NO: 713 (PKKKRKV). In some embodiments, the Cas9 protein (e.g., the SaCas9 or SluCas9 protein) is fused to one or more nucleoplasmin NLSs. In some embodiments, the Cas protein is fused to one or more c-myc NLSs. In some embodiments, the Cas protein is fused to one or more E1A NLSs. In some embodiments, the Cas protein is fused to one or more BP (bipartite) NLSs. In some embodiments, the nucleoplasmin NLS comprises the amino acid sequence of SEQ ID NO: 714 (KRPAATKKAGQAKKKK). In some embodiments, the Cas9 protein is fused with a c-Myc NLS. In some embodiments, the c-Myc NLS comprises the amino acid sequence of SEQ ID NO: 942 (PAAKKKKLD) and/or is encoded by the nucleic acid sequence of SEQ ID NO: 722 (CCGGCAGCTAAGAAAAAGAAACTGGAT). In some embodiments, the Cas9 is fused to two SV40 NLS sequences linked at the carboxy terminus. In some embodiments, the Cas9 may be fused with two NLSs, one linked at the N-terminus and one at the C-terminus. In some embodiments, the Cas9 may be fused with 3 NLSs. In some embodiments, the Cas9 may be fused with 3 NLSs, two linked at the N-terminus and one linked at the C-terminus. In some embodiments, the Cas9 may be fused with 3 NLSs, one linked at the N-terminus and two linked at the C-terminus. In some embodiments, the Cas9 may be fused with no NLS. In some embodiments, the Cas9 may be fused with one NLS. In some embodiments, the Cas9 may be fused with an NLS on the C-terminus and does not comprise an NLS fused on the N-terminus. In some embodiments, the Cas9 may be fused with an NLS on the N-terminus and does not comprise an NLS fused on the C-terminus. In some embodiments, the Cas9 protein is fused to an SV40 NLS and to a nucleoplasmin NLS. In some embodiments, the Cas9 protein is fused to an SV40 NLS and to a c-Myc NLS. In some embodiments, the SV40 NLS is fused to the C-terminus of the Cas9, while the nucleoplasmin NLS is fused to the N-terminus of the Cas9 protein. In some embodiments, the SV40 NLS is fused to the C-terminus of the Cas9, while the c-Myc NLS is fused to the N-terminus of the Cas9 protein. In some embodiments, the SV40 NLS is fused to the N-terminus of the Cas9, while the nucleoplasmin NLS is fused to the C-terminus of the Cas9 protein. In some embodiments, the SV40 NLS is fused to the N-terminus of the Cas9, while the c-Myc NLS is fused to the C-terminus of the Cas9 protein. In some embodiments, the SV40 NLS is fused to the Cas9 protein by means of a linker. In some embodiments, the SV40 NLS and linker is encoded by the nucleic acid sequence of SEQ ID NO: 723 (ATGATGGCCCCAAAGAAGAAGCGGAAGGTCGGTATCCACGGAGTCCCAGCAGCC). In some embodiments, the nucleoplasmin NLS is fused to the Cas9 protein by means of a linker. In some embodiments, the c-Myc NLS is fused to the Cas9 protein by means of a linker. In some embodiments, an additional domain may be: a) fused to the N- or C-terminus of the Cas protein (e.g., a Cas9 protein), b) fused to the N-terminus of an NLS fused to the N-terminus of a Cas protein, or c) fused to the C-terminus of an NLS fused to the C-terminus of a Cas protein. In some embodiments, an NLS is fused to the N- and/or C-terminus of the Cas protein by means of a linker. In some embodiments, an NLS is fused to the N-terminus of an N-terminally-fused NLS on a Cas protein by means of a linker, and/or an NLS is fused to the C-terminus of a C-terminally fused NLS on a Cas protein by means of a linker. In some embodiments, the linker is GSVD (SEQ ID NO: 550) or GSGS (SEQ ID NO: 551). In some embodiments, the Cas protein comprises a c-Myc NLS fused to the N-terminus of the Cas protein (or to an N-terminally-fused NLS on the Cas protein), optionally by means of a linker. In some embodiments, the Cas protein comprises an SV40 NLS fused to the C-terminus of the Cas protein (or to a C-terminally-fused NLS on the Cas protein), optionally by means of a linker. In some embodiments, the Cas protein comprises a nucleoplasmin NLS fused to the C-terminus of the Cas protein (or to a C-terminally-fused NLS on the Cas protein), optionally by means of a linker. In some embodiments, the Cas protein comprises: a) a c-Myc NLS fused to the N-terminus of the Cas protein, optionally by means of a linker, b) an SV40 NLS fused to the C-terminus of the Cas protein, optionally by means of a linker, and c) a nucleoplasmin NLS fused to the C-terminus of the SV40 NLS, optionally by means of a linker. In some embodiments, the Cas protein comprises: a) a c-Myc NLS fused to the N-terminus of the Cas protein, optionally by means of a linker, b) a nucleoplasmin NLS fused to the C-terminus of the Cas protein, optionally by means of a linker, and c) an SV40 NLS fused to the C-terminus of the nucleoplasmin NLS, optionally by means of a linker. In some embodiments, a c-myc NLS is fused to the N-terminus of the Cas9 and an SV40 NLS and/or nucleoplasmin NLS is fused to the C-terminus of the Cas9. In some embodiments, a c-myc NLS is fused to the N-terminus of the Cas9 (e.g., by means of a linker such as GSVD), an SV40 NLS is fused to the C-terminus of the Cas9 (e.g., by means of a linker such as GSGS), and a nucleoplasmin NLS is fused to the C-terminus of the SV-40 NLS (e.g., by means of a linker such as GSGS).

In some embodiments, the heterologous functional domain may be capable of modifying the intracellular half-life of the Cas9. In some embodiments, the half-life of the Cas9 may be increased. In some embodiments, the half-life of the Cas9 may be reduced. In some embodiments, the heterologous functional domain may be capable of increasing the stability of the Cas9. In some embodiments, the heterologous functional domain may be capable of reducing the stability of the Cas9. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the Cas9 may be modified by addition of ubiquitin or a polyubiquitin chain. In some embodiments, the ubiquitin may be a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called Rub1 in S. cerevisiae), human leukocyte antigen F-associated (FAT10), autophagy-8 (ATG8) and −12 (ATG12), Fau ubiquitin-like protein (FUB1), membrane-anchored UBL (MUB), ubiquitin fold-modifier-I (UFM1), and ubiquitin-like protein-5 (UBL5).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Non-limiting examples of suitable fluorescent proteins include green fluorescent proteins (e.g., GFP, GFP-2, tagGFP, turboGFP, sfGFP, EGFP, Emerald, Azami Green, Monomeric Azami Green, CopGFP, AceGFP, ZsGreenl), yellow fluorescent proteins (e.g., YFP, EYFP, Citrine, Venus, YPet, PhiYFP, ZsYellowl), blue fluorescent proteins (e.g., EBFP, EBFP2, Azurite, mKalamal, GFPuv, Sapphire, T-sapphire,), cyan fluorescent proteins (e.g., ECFP, Cerulean, CyPet, AmCyanl, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRedl, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain may be a purification tag and/or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AU1, AUS, E, ECS, E2, FLAG, HA, nus, Softag 1, Softag 3, Strep, SBP, Glu-Glu, HSV, KT3, S, S1, T7, V5, VSV-G, 6×His, 8×His, biotin carboxyl carrier protein (BCCP), poly-His, and calmodulin. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the heterologous functional domain may target the Cas9 to a specific organelle, cell type, tissue, or organ. In some embodiments, the heterologous functional domain may target the Cas9 to muscle.

In further embodiments, the heterologous functional domain may be an effector domain. When the Cas9 is directed to its target sequence, e.g., when a Cas9 is directed to a target sequence by a guide RNA, the effector domain may modify or affect the target sequence. In some embodiments, the effector domain may be chosen from a nucleic acid binding domain or a nuclease domain (e.g., a non-Cas nuclease domain). In some embodiments, the heterologous functional domain is a nuclease, such as a FokI nuclease. See, e.g., U.S. Pat. No. 9,023,649.

In some embodiments, any of the compositions disclosed herein comprising any of the guides and/or endonucleases disclosed herein is sterile and/or substantially pyrogen-free. In particular embodiments, any of the compositions disclosed herein comprise a pharmaceutically acceptable carrier. The phrase “pharmaceutically or pharmacologically acceptable” refers to molecular entities and compositions that do not produce an adverse, allergic, or other untoward reaction when administered to an animal or human. As used herein “pharmaceutically acceptable carrier” includes any and all solvents (e.g., water), dispersion media, coatings, antibacterial and antifungal agents, isotonic and absorption delaying agents, and the like that are physiologically compatible, including pharmaceutically acceptable cell culture media. Pharmaceutically acceptable carriers include sterile aqueous solutions or dispersions and sterile powders for the extemporaneous preparation of sterile injectable solutions or dispersion. In some embodiments, the composition comprises a preservative to prevent the growth of microorganisms.

### Determination of Efficacy of Guide RNAs

In some embodiments, the efficacy of a guide RNA is determined when delivered or expressed together with other components forming an RNP. In some embodiments, the guide RNA is expressed together with a SaCas9 or SluCas9. In some embodiments, the guide RNA is delivered to or expressed in a cell line that already stably expresses an SaCas9 or SluCas9. In some embodiments the guide RNA is delivered to a cell as part of an RNP. In some embodiments, the guide RNA is delivered to a cell along with a nucleic acid (e.g., mRNA) encoding SaCas9 or SluCas9.

In some embodiments, the efficacy of particular guide RNAs is determined based on in vitro models. In some embodiments, the in vitro model is a cell line.

In some embodiments, the efficacy of particular guide RNAs is determined across multiple in vitro cell models for a guide RNA selection process. In some embodiments, a cell line comparison of data with selected guide RNAs is performed. In some embodiments, cross screening in multiple cell models is performed.

In some embodiments, the efficacy of particular guide RNAs is determined based on in vivo models. In some embodiments, the in vivo model is a rodent model. In some embodiments, the rodent model is a mouse which expresses a mutated dystrophin gene. In some embodiments, the in vivo model is a non-human primate, for example cynomolgus monkey.

### III. Methods of Gene Editing and Treating DMD

This disclosure provides methods for gene editing and treating Duchenne Muscular Dystrophy (DMD). In some embodiments, any of the compositions described herein may be administered to a subject in need thereof for use in making a double or single strand break, or excising a portion (e.g., less than about 250 nucleotides) in any one or more of exons 44, 45, 50, 51, or 53 of the dystrophin (DMD) gene and to treat DMD. In some embodiments, pairs of guide RNAs described herein, in any of the vector configurations described herein, may be administered to a subject in need thereof to make a single or double-strand break, excise a portion of a DMD, and treat DMD. In some embodiments, any of the compositions described herein may be administered to a subject in need thereof for use in treating DMD exon. In some embodiments, a nucleic acid molecule comprising a first nucleic acid encoding one or more guide RNAs of Table 6 and a second nucleic acid encoding either SaCas9 or SluCas9 (depending on the guide) is administered to a subject to treat DMD. In some embodiments, a single nucleic acid molecule (which may be a vector, including an AAV vector) comprising a first nucleic acid encoding one or more guide RNAs of Table 6 and a second nucleic acid encoding either SaCas9 or SluCas9 (depending on the guide) is administered to a subject to treat DMD. In some embodiments, more than one nucleic acid molecules (which may be a vectors, including an AAV vectors) are administered to a subject to treat DMD, wherein a first nucleic acid encoding one or more guide RNAs of Table 6 and a second nucleic acid encoding either SaCas9 or SluCas9 (depending on the guide) are together on one nucleic acid molecule or separated on different nucleic acid molecules.

In some embodiments, any of the compositions described herein is administered to a subject in need thereof to treat Duchenne Muscular Dystrophy (DMD).

In some embodiments, any of the compositions described herein is administered to a subject in need thereof to induce a double or single strand break in any one or more of exons 44, 45, 50, 51, or 53 of the dystrophin gene.

In some embodiments, any of the compositions described herein is administered to a subject in need thereof to delete a portion (e.g., excise a portion) any one of exons 44, 45, 50, 51, or 53 of the dystrophin gene.

In some embodiments, a method of treating Duchenne Muscular Dystrophy (DMD) is provided, the method comprising delivering to a cell any one of the compositions described herein, wherein the cell comprises a mutation in the dystrophin gene that is known to be associated with DMD.

In some embodiments, methods are provided for treating Duchenne Muscular Dystrophy (DMD), the method comprising delivering to a cell at least one nucleic acid molecule comprising a pair of guide RNAs comprising a first and second spacer sequence, wherein the pair of first and spacer sequences are selected from any one of the following pairs: SEQ ID NO: 1 and SEQ ID NO: 3; SEQ ID NO: 3 and SEQ ID NO: 4; SEQ ID NO: 3 and SEQ ID NO: 5; SEQ ID NO: 3 and SEQ ID NO: 6; SEQ ID NO: 3 and SEQ ID NO: 7; SEQ ID NO: 3 and SEQ ID NO: 8; SEQ ID NO: 3 and SEQ ID NO: 9; SEQ ID NO: 4 and SEQ ID NO: 5; SEQ ID NO: 4 and SEQ ID NO: 6; SEQ ID NO: 4 and SEQ ID NO: 7; SEQ ID NO: 4 and SEQ ID NO: 8; SEQ ID NO: 4 and SEQ ID NO: 9; SEQ ID NO: 5 and SEQ ID NO: 6; SEQ ID NO: 5 and SEQ ID NO: 7; SEQ ID NO: 5 and SEQ ID NO: 8; SEQ ID NO: 5 and SEQ ID NO: 9; SEQ ID NO: 6 and SEQ ID NO: 7; SEQ ID NO: 6 and SEQ ID NO: 8; SEQ ID NO: 6 and SEQ ID NO: 9; SEQ ID NO: 7 and SEQ ID NO: 8; SEQ ID NO: 7 and SEQ ID NO: 9; SEQ ID NO: 8 and SEQ ID NO: 9; SEQ ID NO: 11 and SEQ ID NO: 12; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 13 and SEQ ID NO: 14; SEQ ID NO: 13 and SEQ ID NO: 15; SEQ ID NO: 14 and SEQ ID NO: 15; SEQ ID NO: 16 and SEQ ID NO: 17; SEQ ID NO: 16 and SEQ ID NO: 18; SEQ ID NO: 16 and SEQ ID NO: 19; SEQ ID NO: 16 and SEQ ID NO: 20; SEQ ID NO: 16 and SEQ ID NO: 21; SEQ ID NO: 16 and SEQ ID NO: 22; SEQ ID NO: 16 and SEQ ID NO: 23; SEQ ID NO: 16 and SEQ ID NO: 24; SEQ ID NO: 16 and SEQ ID NO: 25; SEQ ID NO: 16 and SEQ ID NO: 26; SEQ ID NO: 17 and SEQ ID NO: 18; SEQ ID NO: 17 and SEQ ID NO: 19; SEQ ID NO: 17 and SEQ ID NO: 20; SEQ ID NO: 17 and SEQ ID NO: 21; SEQ ID NO: 17 and SEQ ID NO: 22; SEQ ID NO: 17 and SEQ ID NO: 23; SEQ ID NO: 17 and SEQ ID NO: 24; SEQ ID NO: 17 and SEQ ID NO: 25; SEQ ID NO: 17 and SEQ ID NO: 26; SEQ ID NO: 18 and SEQ ID NO: 19; SEQ ID NO: 18 and SEQ ID NO: 20; SEQ ID NO: 18 and SEQ ID NO: 21; SEQ ID NO: 18 and SEQ ID NO: 22; SEQ ID NO: 18 and SEQ ID NO: 23; SEQ ID NO: 18 and SEQ ID NO: 24; SEQ ID NO: 18 and SEQ ID NO: 25; SEQ ID NO: 18 and SEQ ID NO: 26; SEQ ID NO: 19 and SEQ ID NO: 20; SEQ ID NO: 19 and SEQ ID NO: 21; SEQ ID NO: 19 and SEQ ID NO: 22; SEQ ID NO: 19 and SEQ ID NO: 23; SEQ ID NO: 19 and SEQ ID NO: 24; SEQ ID NO: 19 and SEQ ID NO: 25; SEQ ID NO: 19 and SEQ ID NO: 26; SEQ ID NO: 20 and SEQ ID NO: 21; SEQ ID NO: 20 and SEQ ID NO: 22; SEQ ID NO: 20 and SEQ ID NO: 23; SEQ ID NO: 20 and SEQ ID NO: 24; SEQ ID NO: 20 and SEQ ID NO: 25; SEQ ID NO: 20 and SEQ ID NO: 26; SEQ ID NO: 21 and SEQ ID NO: 22; SEQ ID NO: 21 and SEQ ID NO: 23; SEQ ID NO: 21 and SEQ ID NO: 24; SEQ ID NO: 21 and SEQ ID NO: 25; SEQ ID NO: 21 and SEQ ID NO: 26; SEQ ID NO: 22 and SEQ ID NO: 23; SEQ ID NO: 22 and SEQ ID NO: 24; SEQ ID NO: 22 and SEQ ID NO: 25; SEQ ID NO: 22 and SEQ ID NO: 26; SEQ ID NO: 23 and SEQ ID NO: 24; SEQ ID NO: 23 and SEQ ID NO: 25; SEQ ID NO: 23 and SEQ ID NO: 26; SEQ ID NO: 24 and SEQ ID NO: 25; SEQ ID NO: 24 and SEQ ID NO: 26; SEQ ID NO: 25 and SEQ ID NO: 26; SEQ ID NO: 110 and SEQ ID NO: 120; SEQ ID NO: 110 and SEQ ID NO: 121; SEQ ID NO: 110 and SEQ ID NO: 122; SEQ ID NO: 110 and SEQ ID NO: 123; SEQ ID NO: 110 and SEQ ID NO: 124; SEQ ID NO: 110 and SEQ ID NO: 125; SEQ ID NO: 111 and SEQ ID NO: 112; SEQ ID NO: 111 and SEQ ID NO: 113; SEQ ID NO: 111 and SEQ ID NO: 114; SEQ ID NO: 111 and SEQ ID NO: 115; SEQ ID NO: 111 and SEQ ID NO: 116; SEQ ID NO: 111 and SEQ ID NO: 117; SEQ ID NO: 111 and SEQ ID NO: 118; SEQ ID NO: 111 and SEQ ID NO: 119; SEQ ID NO: 111 and SEQ ID NO: 120; SEQ ID NO: 111 and SEQ ID NO: 121; SEQ ID NO: 111 and SEQ ID NO: 122; SEQ ID NO: 111 and SEQ ID NO: 123; SEQ ID NO: 111 and SEQ ID NO: 124; SEQ ID NO: 111 and SEQ ID NO: 125; SEQ ID NO: 112 and SEQ ID NO: 113; SEQ ID NO: 112 and SEQ ID NO: 114; SEQ ID NO: 112 and SEQ ID NO: 115; SEQ ID NO: 112 and SEQ ID NO: 116; SEQ ID NO: 112 and SEQ ID NO: 117; SEQ ID NO: 112 and SEQ ID NO: 118; SEQ ID NO: 112 and SEQ ID NO: 119; SEQ ID NO: 112 and SEQ ID NO: 120; SEQ ID NO: 112 and SEQ ID NO: 121; SEQ ID NO: 112 and SEQ ID NO: 122; SEQ ID NO: 112 and SEQ ID NO: 123; SEQ ID NO: 112 and SEQ ID NO: 124; SEQ ID NO: 112 and SEQ ID NO: 125; SEQ ID NO: 113 and SEQ ID NO: 114; SEQ ID NO: 113 and SEQ ID NO: 115; SEQ ID NO: 113 and SEQ ID NO: 116; SEQ ID NO: 113 and SEQ ID NO: 117; SEQ ID NO: 113 and SEQ ID NO: 118; SEQ ID NO: 113 and SEQ ID NO: 119; SEQ ID NO: 113 and SEQ ID NO: 120; SEQ ID NO: 113 and SEQ ID NO: 121; SEQ ID NO: 113 and SEQ ID NO: 122; SEQ ID NO: 113 and SEQ ID NO: 123; SEQ ID NO: 113 and SEQ ID NO: 124; SEQ ID NO: 113 and SEQ ID NO: 125; SEQ ID NO: 114 and SEQ ID NO: 115; SEQ ID NO: 114 and SEQ ID NO: 116; SEQ ID NO: 114 and SEQ ID NO: 117; SEQ ID NO: 114 and SEQ ID NO: 118; SEQ ID NO: 114 and SEQ ID NO: 119; SEQ ID NO: 114 and SEQ ID NO: 120; SEQ ID NO: 114 and SEQ ID NO: 121; SEQ ID NO: 114 and SEQ ID NO: 122; SEQ ID NO: 114 and SEQ ID NO: 123; SEQ ID NO: 114 and SEQ ID NO: 124; SEQ ID NO: 114 and SEQ ID NO: 125; SEQ ID NO: 115 and SEQ ID NO: 116; SEQ ID NO: 115 and SEQ ID NO: 117; SEQ ID NO: 115 and SEQ ID NO: 118; SEQ ID NO: 115 and SEQ ID NO: 119; SEQ ID NO: 115 and SEQ ID NO: 120; SEQ ID NO: 115 and SEQ ID NO: 121; SEQ ID NO: 115 and SEQ ID NO: 122; SEQ ID NO: 115 and SEQ ID NO: 123; SEQ ID NO: 115 and SEQ ID NO: 124; SEQ ID NO: 115 and SEQ ID NO: 125; SEQ ID NO: 116 and SEQ ID NO: 117; SEQ ID NO: 116 and SEQ ID NO: 118; SEQ ID NO: 116 and SEQ ID NO: 119; SEQ ID NO: 116 and SEQ ID NO: 120; SEQ ID NO: 116 and SEQ ID NO: 121; SEQ ID NO: 116 and SEQ ID NO: 122; SEQ ID NO: 116 and SEQ ID NO: 123; SEQ ID NO: 116 and SEQ ID NO: 124; SEQ ID NO: 116 and SEQ ID NO: 125; SEQ ID NO: 117 and SEQ ID NO: 118; SEQ ID NO: 117 and SEQ ID NO: 119; SEQ ID NO: 117 and SEQ ID NO: 120; SEQ ID NO: 117 and SEQ ID NO: 121; SEQ ID NO: 117 and SEQ ID NO: 122; SEQ ID NO: 117 and SEQ ID NO: 123; SEQ ID NO: 117 and SEQ ID NO: 124; SEQ ID NO: 117 and SEQ ID NO: 125; SEQ ID NO: 118 and SEQ ID NO: 119; SEQ ID NO: 118 and SEQ ID NO: 120; SEQ ID NO: 118 and SEQ ID NO: 121; SEQ ID NO: 118 and SEQ ID NO: 122; SEQ ID NO: 118 and SEQ ID NO: 123; SEQ ID NO: 118 and SEQ ID NO: 124; SEQ ID NO: 118 and SEQ ID NO: 125; SEQ ID NO: 119 and SEQ ID NO: 120; SEQ ID NO: 119 and SEQ ID NO: 121; SEQ ID NO: 119 and SEQ ID NO: 122; SEQ ID NO: 119 and SEQ ID NO: 123; SEQ ID NO: 119 and SEQ ID NO: 124; SEQ ID NO: 119 and SEQ ID NO: 125; SEQ ID NO: 120 and SEQ ID NO: 121; SEQ ID NO: 120 and SEQ ID NO: 122; SEQ ID NO: 120 and SEQ ID NO: 123; SEQ ID NO: 120 and SEQ ID NO: 124; SEQ ID NO: 120 and SEQ ID NO: 125; SEQ ID NO: 121 and SEQ ID NO: 122; SEQ ID NO: 121 and SEQ ID NO: 123; SEQ ID NO: 121 and SEQ ID NO: 124; SEQ ID NO: 121 and SEQ ID NO: 125; SEQ ID NO: 122 and SEQ ID NO: 123; SEQ ID NO: 122 and SEQ ID NO: 124; SEQ ID NO: 122 and SEQ ID NO: 125; SEQ ID NO: 123 and SEQ ID NO: 124; SEQ ID NO: 123 and SEQ ID NO: 125; SEQ ID NO: 124 and SEQ ID NO: 125; SEQ ID NO: 126 and SEQ ID NO: 127; SEQ ID NO: 126 and SEQ ID NO: 128; SEQ ID NO: 126 and SEQ ID NO: 129; SEQ ID NO: 126 and SEQ ID NO: 130; SEQ ID NO: 126 and SEQ ID NO: 131; SEQ ID NO: 126 and SEQ ID NO: 132; SEQ ID NO: 126 and SEQ ID NO: 133; SEQ ID NO: 126 and SEQ ID NO: 134; SEQ ID NO: 126 and SEQ ID NO: 135; SEQ ID NO: 126 and SEQ ID NO: 136; SEQ ID NO: 126 and SEQ ID NO: 137; SEQ ID NO: 126 and SEQ ID NO: 138; SEQ ID NO: 126 and SEQ ID NO: 139; SEQ ID NO: 126 and SEQ ID NO: 140; SEQ ID NO: 126 and SEQ ID NO: 141; SEQ ID NO: 126 and SEQ ID NO: 142; SEQ ID NO: 126 and SEQ ID NO: 143; SEQ ID NO: 126 and SEQ ID NO: 144; SEQ ID NO: 126 and SEQ ID NO: 145; SEQ ID NO: 126 and SEQ ID NO: 146; SEQ ID NO: 126 and SEQ ID NO: 147; SEQ ID NO: 127 and SEQ ID NO: 128; SEQ ID NO: 127 and SEQ ID NO: 129; SEQ ID NO: 127 and SEQ ID NO: 130; SEQ ID NO: 127 and SEQ ID NO: 131; SEQ ID NO: 127 and SEQ ID NO: 132; SEQ ID NO: 127 and SEQ ID NO: 133; SEQ ID NO: 127 and SEQ ID NO: 134; SEQ ID NO: 127 and SEQ ID NO: 135; SEQ ID NO: 127 and SEQ ID NO: 136; SEQ ID NO: 127 and SEQ ID NO: 137; SEQ ID NO: 127 and SEQ ID NO: 138; SEQ ID NO: 127 and SEQ ID NO: 139; SEQ ID NO: 127 and SEQ ID NO: 140; SEQ ID NO: 127 and SEQ ID NO: 141; SEQ ID NO: 127 and SEQ ID NO: 142; SEQ ID NO: 127 and SEQ ID NO: 143; SEQ ID NO: 127 and SEQ ID NO: 144; SEQ ID NO: 127 and SEQ ID NO: 145; SEQ ID NO: 127 and SEQ ID NO: 146; SEQ ID NO: 127 and SEQ ID NO: 147; SEQ ID NO: 128 and SEQ ID NO: 129; SEQ ID NO: 128 and SEQ ID NO: 130; SEQ ID NO: 128 and SEQ ID NO: 131; SEQ ID NO: 128 and SEQ ID NO: 132; SEQ ID NO: 128 and SEQ ID NO: 133; SEQ ID NO: 128 and SEQ ID NO: 134; SEQ ID NO: 128 and SEQ ID NO: 135; SEQ ID NO: 128 and SEQ ID NO: 136; SEQ ID NO: 128 and SEQ ID NO: 137; SEQ ID NO: 128 and SEQ ID NO: 138; SEQ ID NO: 128 and SEQ ID NO: 139; SEQ ID NO: 128 and SEQ ID NO: 140; SEQ ID NO: 128 and SEQ ID NO: 141; SEQ ID NO: 128 and SEQ ID NO: 142; SEQ ID NO: 128 and SEQ ID NO: 143; SEQ ID NO: 128 and SEQ ID NO: 144; SEQ ID NO: 128 and SEQ ID NO: 145; SEQ ID NO: 128 and SEQ ID NO: 146; SEQ ID NO: 128 and SEQ ID NO: 147; SEQ ID NO: 129 and SEQ ID NO: 130; SEQ ID NO: 129 and SEQ ID NO: 131; SEQ ID NO: 129 and SEQ ID NO: 132; SEQ ID NO: 129 and SEQ ID NO: 133; SEQ ID NO: 129 and SEQ ID NO: 134; SEQ ID NO: 129 and SEQ ID NO: 135; SEQ ID NO: 129 and SEQ ID NO: 136; SEQ ID NO: 129 and SEQ ID NO: 137; SEQ ID NO: 129 and SEQ ID NO: 138; SEQ ID NO: 129 and SEQ ID NO: 139; SEQ ID NO: 129 and SEQ ID NO: 140; SEQ ID NO: 129 and SEQ ID NO: 141; SEQ ID NO: 129 and SEQ ID NO: 142; SEQ ID NO: 129 and SEQ ID NO: 143; SEQ ID NO: 129 and SEQ ID NO: 144; SEQ ID NO: 129 and SEQ ID NO: 145; SEQ ID NO: 129 and SEQ ID NO: 146; SEQ ID NO: 129 and SEQ ID NO: 147; SEQ ID NO: 130 and SEQ ID NO: 131; SEQ ID NO: 130 and SEQ ID NO: 132; SEQ ID NO: 130 and SEQ ID NO: 133; SEQ ID NO: 130 and SEQ ID NO: 134; SEQ ID NO: 130 and SEQ ID NO: 135; SEQ ID NO: 130 and SEQ ID NO: 136; SEQ ID NO: 130 and SEQ ID NO: 137; SEQ ID NO: 130 and SEQ ID NO: 138; SEQ ID NO: 130 and SEQ ID NO: 139; SEQ ID NO: 130 and SEQ ID NO: 140; SEQ ID NO: 130 and SEQ ID NO: 141; SEQ ID NO: 130 and SEQ ID NO: 142; SEQ ID NO: 130 and SEQ ID NO: 143; SEQ ID NO: 130 and SEQ ID NO: 144; SEQ ID NO: 130 and SEQ ID NO: 145; SEQ ID NO: 130 and SEQ ID NO: 146; SEQ ID NO: 130 and SEQ ID NO: 147; SEQ ID NO: 131 and SEQ ID NO: 132; SEQ ID NO: 131 and SEQ ID NO: 133; SEQ ID NO: 131 and SEQ ID NO: 134; SEQ ID NO: 131 and SEQ ID NO: 135; SEQ ID NO: 131 and SEQ ID NO: 136; SEQ ID NO: 131 and SEQ ID NO: 137; SEQ ID NO: 131 and SEQ ID NO: 138; SEQ ID NO: 131 and SEQ ID NO: 139; SEQ ID NO: 131 and SEQ ID NO: 140; SEQ ID NO: 131 and SEQ ID NO: 141; SEQ ID NO: 131 and SEQ ID NO: 142; SEQ ID NO: 131 and SEQ ID NO: 143; SEQ ID NO: 131 and SEQ ID NO: 144; SEQ ID NO: 131 and SEQ ID NO: 145; SEQ ID NO: 131 and SEQ ID NO: 146; SEQ ID NO: 131 and SEQ ID NO: 147; SEQ ID NO: 132 and SEQ ID NO: 133; SEQ ID NO: 132 and SEQ ID NO: 134; SEQ ID NO: 132 and SEQ ID NO: 135; SEQ ID NO: 132 and SEQ ID NO: 136; SEQ ID NO: 132 and SEQ ID NO: 137; SEQ ID NO: 132 and SEQ ID NO: 138; SEQ ID NO: 132 and SEQ ID NO: 139; SEQ ID NO: 132 and SEQ ID NO: 140; SEQ ID NO: 132 and SEQ ID NO: 141; SEQ ID NO: 132 and SEQ ID NO: 142; SEQ ID NO: 132 and SEQ ID NO: 143; SEQ ID NO: 132 and SEQ ID NO: 144; SEQ ID NO: 132 and SEQ ID NO: 145; SEQ ID NO: 132 and SEQ ID NO: 146; SEQ ID NO: 132 and SEQ ID NO: 147; SEQ ID NO: 133 and SEQ ID NO: 134; SEQ ID NO: 133 and SEQ ID NO: 135; SEQ ID NO: 133 and SEQ ID NO: 136; SEQ ID NO: 133 and SEQ ID NO: 137; SEQ ID NO: 133 and SEQ ID NO: 138; SEQ ID NO: 133 and SEQ ID NO: 139; SEQ ID NO: 133 and SEQ ID NO: 140; SEQ ID NO: 133 and SEQ ID NO: 141; SEQ ID NO: 133 and SEQ ID NO: 142; SEQ ID NO: 133 and SEQ ID NO: 143; SEQ ID NO: 133 and SEQ ID NO: 144; SEQ ID NO: 133 and SEQ ID NO: 145; SEQ ID NO: 133 and SEQ ID NO: 146; SEQ ID NO: 133 and SEQ ID NO: 147; SEQ ID NO: 134 and SEQ ID NO: 135; SEQ ID NO: 134 and SEQ ID NO: 136; SEQ ID NO: 134 and SEQ ID NO: 137; SEQ ID NO: 134 and SEQ ID NO: 138; SEQ ID NO: 134 and SEQ ID NO: 139; SEQ ID NO: 134 and SEQ ID NO: 140; SEQ ID NO: 134 and SEQ ID NO: 141; SEQ ID NO: 134 and SEQ ID NO: 142; SEQ ID NO: 134 and SEQ ID NO: 143; SEQ ID NO: 134 and SEQ ID NO: 144; SEQ ID NO: 134 and SEQ ID NO: 145; SEQ ID NO: 134 and SEQ ID NO: 146; SEQ ID NO: 134 and SEQ ID NO: 147; SEQ ID NO: 135 and SEQ ID NO: 136; SEQ ID NO: 135 and SEQ ID NO: 137; SEQ ID NO: 135 and SEQ ID NO: 138; SEQ ID NO: 135 and SEQ ID NO: 139; SEQ ID NO: 135 and SEQ ID NO: 140; SEQ ID NO: 135 and SEQ ID NO: 141; SEQ ID NO: 135 and SEQ ID NO: 142; SEQ ID NO: 135 and SEQ ID NO: 143; SEQ ID NO: 135 and SEQ ID NO: 144; SEQ ID NO: 135 and SEQ ID NO: 145; SEQ ID NO: 135 and SEQ ID NO: 146; SEQ ID NO: 135 and SEQ ID NO: 147; SEQ ID NO: 136 and SEQ ID NO: 137; SEQ ID NO: 136 and SEQ ID NO: 138; SEQ ID NO: 136 and SEQ ID NO: 139; SEQ ID NO: 136 and SEQ ID NO: 140; SEQ ID NO: 136 and SEQ ID NO: 141; SEQ ID NO: 136 and SEQ ID NO: 142; SEQ ID NO: 136 and SEQ ID NO: 143; SEQ ID NO: 136 and SEQ ID NO: 144; SEQ ID NO: 136 and SEQ ID NO: 145; SEQ ID NO: 136 and SEQ ID NO: 146; SEQ ID NO: 136 and SEQ ID NO: 147; SEQ ID NO: 137 and SEQ ID NO: 138; SEQ ID NO: 137 and SEQ ID NO: 139; SEQ ID NO: 137 and SEQ ID NO: 140; SEQ ID NO: 137 and SEQ ID NO: 141; SEQ ID NO: 137 and SEQ ID NO: 142; SEQ ID NO: 137 and SEQ ID NO: 143; SEQ ID NO: 137 and SEQ ID NO: 144; SEQ ID NO: 137 and SEQ ID NO: 145; SEQ ID NO: 137 and SEQ ID NO: 146; SEQ ID NO: 137 and SEQ ID NO: 147; SEQ ID NO: 138 and SEQ ID NO: 139; SEQ ID NO: 138 and SEQ ID NO: 140; SEQ ID NO: 138 and SEQ ID NO: 141; SEQ ID NO: 138 and SEQ ID NO: 142; SEQ ID NO: 138 and SEQ ID NO: 143; SEQ ID NO: 138 and SEQ ID NO: 144; SEQ ID NO: 138 and SEQ ID NO: 145; SEQ ID NO: 138 and SEQ ID NO: 146; SEQ ID NO: 138 and SEQ ID NO: 147; SEQ ID NO: 139 and SEQ ID NO: 140; SEQ ID NO: 139 and SEQ ID NO: 141; SEQ ID NO: 139 and SEQ ID NO: 142; SEQ ID NO: 139 and SEQ ID NO: 143; SEQ ID NO: 139 and SEQ ID NO: 144; SEQ ID NO: 139 and SEQ ID NO: 145; SEQ ID NO: 139 and SEQ ID NO: 146; SEQ ID NO: 139 and SEQ ID NO: 147; SEQ ID NO: 140 and SEQ ID NO: 141; SEQ ID NO: 140 and SEQ ID NO: 142; SEQ ID NO: 140 and SEQ ID NO: 143; SEQ ID NO: 140 and SEQ ID NO: 144; SEQ ID NO: 140 and SEQ ID NO: 145; SEQ ID NO: 140 and SEQ ID NO: 146; SEQ ID NO: 140 and SEQ ID NO: 147; SEQ ID NO: 141 and SEQ ID NO: 142; SEQ ID NO: 141 and SEQ ID NO: 143; SEQ ID NO: 141 and SEQ ID NO: 144; SEQ ID NO: 141 and SEQ ID NO: 145; SEQ ID NO: 141 and SEQ ID NO: 146; SEQ ID NO: 141 and SEQ ID NO: 147; SEQ ID NO: 142 and SEQ ID NO: 143; SEQ ID NO: 142 and SEQ ID NO: 144; SEQ ID NO: 142 and SEQ ID NO: 145; SEQ ID NO: 142 and SEQ ID NO: 146; SEQ ID NO: 142 and SEQ ID NO: 147; SEQ ID NO: 143 and SEQ ID NO: 144; SEQ ID NO: 143 and SEQ ID NO: 145; SEQ ID NO: 143 and SEQ ID NO: 146; SEQ ID NO: 143 and SEQ ID NO: 147; SEQ ID NO: 144 and SEQ ID NO: 145; SEQ ID NO: 144 and SEQ ID NO: 146; SEQ ID NO: 144 and SEQ ID NO: 147; SEQ ID NO: 145 and SEQ ID NO: 146; SEQ ID NO: 145 and SEQ ID NO: 147; SEQ ID NO: 146 and SEQ ID NO: 147; SEQ ID NO: 148 and SEQ ID NO: 149; SEQ ID NO: 148 and SEQ ID NO: 150; SEQ ID NO: 148 and SEQ ID NO: 151; SEQ ID NO: 148 and SEQ ID NO: 152; SEQ ID NO: 148 and SEQ ID NO: 153; SEQ ID NO: 148 and SEQ ID NO: 154; SEQ ID NO: 148 and SEQ ID NO: 155; SEQ ID NO: 148 and SEQ ID NO: 156; SEQ ID NO: 148 and SEQ ID NO: 157; SEQ ID NO: 148 and SEQ ID NO: 158; SEQ ID NO: 148 and SEQ ID NO: 159; SEQ ID NO: 149 and SEQ ID NO: 150; SEQ ID NO: 149 and SEQ ID NO: 151; SEQ ID NO: 149 and SEQ ID NO: 152; SEQ ID NO: 149 and SEQ ID NO: 153; SEQ ID NO: 149 and SEQ ID NO: 154; SEQ ID NO: 149 and SEQ ID NO: 155; SEQ ID NO: 149 and SEQ ID NO: 156; SEQ ID NO: 149 and SEQ ID NO: 157; SEQ ID NO: 149 and SEQ ID NO: 158; SEQ ID NO: 149 and SEQ ID NO: 159; SEQ ID NO: 150 and SEQ ID NO: 151; SEQ ID NO: 150 and SEQ ID NO: 152; SEQ ID NO: 150 and SEQ ID NO: 153; SEQ ID NO: 150 and SEQ ID NO: 154; SEQ ID NO: 150 and SEQ ID NO: 155; SEQ ID NO: 150 and SEQ ID NO: 156; SEQ ID NO: 150 and SEQ ID NO: 157; SEQ ID NO: 150 and SEQ ID NO: 158; SEQ ID NO: 150 and SEQ ID NO: 159; SEQ ID NO: 151 and SEQ ID NO: 152; SEQ ID NO: 151 and SEQ ID NO: 153; SEQ ID NO: 151 and SEQ ID NO: 154; SEQ ID NO: 151 and SEQ ID NO: 155; SEQ ID NO: 151 and SEQ ID NO: 156; SEQ ID NO: 151 and SEQ ID NO: 157; SEQ ID NO: 151 and SEQ ID NO: 158; SEQ ID NO: 151 and SEQ ID NO: 159; SEQ ID NO: 152 and SEQ ID NO: 153; SEQ ID NO: 152 and SEQ ID NO: 154; SEQ ID NO: 152 and SEQ ID NO: 155; SEQ ID NO: 152 and SEQ ID NO: 156; SEQ ID NO: 152 and SEQ ID NO: 157; SEQ ID NO: 152 and SEQ ID NO: 158; SEQ ID NO: 152 and SEQ ID NO: 159; SEQ ID NO: 153 and SEQ ID NO: 154; SEQ ID NO: 153 and SEQ ID NO: 155; SEQ ID NO: 153 and SEQ ID NO: 156; SEQ ID NO: 153 and SEQ ID NO: 157; SEQ ID NO: 153 and SEQ ID NO: 158; SEQ ID NO: 153 and SEQ ID NO: 159; SEQ ID NO: 154 and SEQ ID NO: 155; SEQ ID NO: 154 and SEQ ID NO: 156; SEQ ID NO: 154 and SEQ ID NO: 157; SEQ ID NO: 154 and SEQ ID NO: 158; SEQ ID NO: 154 and SEQ ID NO: 159; SEQ ID NO: 155 and SEQ ID NO: 156; SEQ ID NO: 155 and SEQ ID NO: 157; SEQ ID NO: 155 and SEQ ID NO: 158; SEQ ID NO: 155 and SEQ ID NO: 159; SEQ ID NO: 156 and SEQ ID NO: 157; SEQ ID NO: 156 and SEQ ID NO: 158; SEQ ID NO: 156 and SEQ ID NO: 159; SEQ ID NO: 157 and SEQ ID NO: 158; SEQ ID NO: 157 and SEQ ID NO: 159; SEQ ID NO: 158 and SEQ ID NO: 159; SEQ ID NO: 27 and SEQ ID NO: 28; SEQ ID NO: 27 and SEQ ID NO: 29; SEQ ID NO: 27 and SEQ ID NO: 30; SEQ ID NO: 27 and SEQ ID NO: 31; SEQ ID NO: 27 and SEQ ID NO: 32; SEQ ID NO: 27 and SEQ ID NO: 33; SEQ ID NO: 27 and SEQ ID NO: 34; SEQ ID NO: 27 and SEQ ID NO: 35; SEQ ID NO: 27 and SEQ ID NO: 36; SEQ ID NO: 27 and SEQ ID NO: 37; SEQ ID NO: 27 and SEQ ID NO: 38; SEQ ID NO: 27 and SEQ ID NO: 39; SEQ ID NO: 27 and SEQ ID NO: 40; SEQ ID NO: 27 and SEQ ID NO: 41; SEQ ID NO: 27 and SEQ ID NO: 42; SEQ ID NO: 27 and SEQ ID NO: 43; SEQ ID NO: 27 and SEQ ID NO: 44; SEQ ID NO: 27 and SEQ ID NO: 45; SEQ ID NO: 27 and SEQ ID NO: 46; SEQ ID NO: 27 and SEQ ID NO: 47; SEQ ID NO: 27 and SEQ ID NO: 48; SEQ ID NO: 27 and SEQ ID NO: 53; SEQ ID NO: 27 and SEQ ID NO: 54; SEQ ID NO: 27 and SEQ ID NO: 55; SEQ ID NO: 27 and SEQ ID NO: 56; SEQ ID NO: 27 and SEQ ID NO: 57; SEQ ID NO: 27 and SEQ ID NO: 58; SEQ ID NO: 27 and SEQ ID NO: 59; SEQ ID NO: 27 and SEQ ID NO: 60; SEQ ID NO: 27 and SEQ ID NO: 61; SEQ ID NO: 27 and SEQ ID NO: 62; SEQ ID NO: 27 and SEQ ID NO: 63; SEQ ID NO: 27 and SEQ ID NO: 64; SEQ ID NO: 27 and SEQ ID NO: 65; SEQ ID NO: 27 and SEQ ID NO: 66; SEQ ID NO: 27 and SEQ ID NO: 67; SEQ ID NO: 27 and SEQ ID NO: 68; SEQ ID NO: 27 and SEQ ID NO: 69; SEQ ID NO: 28 and SEQ ID NO: 29; SEQ ID NO: 28 and SEQ ID NO: 30; SEQ ID NO: 28 and SEQ ID NO: 31; SEQ ID NO: 28 and SEQ ID NO: 32; SEQ ID NO: 28 and SEQ ID NO: 33; SEQ ID NO: 28 and SEQ ID NO: 34; SEQ ID NO: 28 and SEQ ID NO: 35; SEQ ID NO: 28 and SEQ ID NO: 36; SEQ ID NO: 28 and SEQ ID NO: 37; SEQ ID NO: 28 and SEQ ID NO: 38; SEQ ID NO: 28 and SEQ ID NO: 39; SEQ ID NO: 28 and SEQ ID NO: 40; SEQ ID NO: 28 and SEQ ID NO: 41; SEQ ID NO: 28 and SEQ ID NO: 42; SEQ ID NO: 28 and SEQ ID NO: 43; SEQ ID NO: 28 and SEQ ID NO: 44; SEQ ID NO: 28 and SEQ ID NO: 45; SEQ ID NO: 28 and SEQ ID NO: 46; SEQ ID NO: 28 and SEQ ID NO: 47; SEQ ID NO: 28 and SEQ ID NO: 48; SEQ ID NO: 28 and SEQ ID NO: 53; SEQ ID NO: 28 and SEQ ID NO: 54; SEQ ID NO: 28 and SEQ ID NO: 55; SEQ ID NO: 28 and SEQ ID NO: 56; SEQ ID NO: 28 and SEQ ID NO: 57; SEQ ID NO: 28 and SEQ ID NO: 58; SEQ ID NO: 28 and SEQ ID NO: 59; SEQ ID NO: 28 and SEQ ID NO: 60; SEQ ID NO: 28 and SEQ ID NO: 61; SEQ ID NO: 28 and SEQ ID NO: 62; SEQ ID NO: 28 and SEQ ID NO: 63; SEQ ID NO: 28 and SEQ ID NO: 64; SEQ ID NO: 28 and SEQ ID NO: 65; SEQ ID NO: 28 and SEQ ID NO: 66; SEQ ID NO: 28 and SEQ ID NO: 67; SEQ ID NO: 28 and SEQ ID NO: 68; SEQ ID NO: 28 and SEQ ID NO: 69; SEQ ID NO: 29 and SEQ ID NO: 30; SEQ ID NO: 29 and SEQ ID NO: 31; SEQ ID NO: 29 and SEQ ID NO: 32; SEQ ID NO: 29 and SEQ ID NO: 33; SEQ ID NO: 29 and SEQ ID NO: 34; SEQ ID NO: 29 and SEQ ID NO: 35; SEQ ID NO: 29 and SEQ ID NO: 36; SEQ ID NO: 29 and SEQ ID NO: 37; SEQ ID NO: 29 and SEQ ID NO: 38; SEQ ID NO: 29 and SEQ ID NO: 39; SEQ ID NO: 29 and SEQ ID NO: 40; SEQ ID NO: 29 and SEQ ID NO: 41; SEQ ID NO: 29 and SEQ ID NO: 42; SEQ ID NO: 29 and SEQ ID NO: 43; SEQ ID NO: 29 and SEQ ID NO: 44; SEQ ID NO: 29 and SEQ ID NO: 45; SEQ ID NO: 29 and SEQ ID NO: 46; SEQ ID NO: 29 and SEQ ID NO: 47; SEQ ID NO: 29 and SEQ ID NO: 48; SEQ ID NO: 29 and SEQ ID NO: 53; SEQ ID NO: 29 and SEQ ID NO: 54; SEQ ID NO: 29 and SEQ ID NO: 55; SEQ ID NO: 29 and SEQ ID NO: 56; SEQ ID NO: 29 and SEQ ID NO: 57; SEQ ID NO: 29 and SEQ ID NO: 58; SEQ ID NO: 29 and SEQ ID NO: 59; SEQ ID NO: 29 and SEQ ID NO: 60; SEQ ID NO: 29 and SEQ ID NO: 61; SEQ ID NO: 29 and SEQ ID NO: 62; SEQ ID NO: 29 and SEQ ID NO: 63; SEQ ID NO: 29 and SEQ ID NO: 64; SEQ ID NO: 29 and SEQ ID NO: 65; SEQ ID NO: 29 and SEQ ID NO: 66; SEQ ID NO: 29 and SEQ ID NO: 67; SEQ ID NO: 29 and SEQ ID NO: 68; SEQ ID NO: 29 and SEQ ID NO: 69; SEQ ID NO: 30 and SEQ ID NO: 31; SEQ ID NO: 30 and SEQ ID NO: 32; SEQ ID NO: 30 and SEQ ID NO: 33; SEQ ID NO: 30 and SEQ ID NO: 34; SEQ ID NO: 30 and SEQ ID NO: 35; SEQ ID NO: 30 and SEQ ID NO: 36; SEQ ID NO: 30 and SEQ ID NO: 37; SEQ ID NO: 30 and SEQ ID NO: 38; SEQ ID NO: 30 and SEQ ID NO: 39; SEQ ID NO: 30 and SEQ ID NO: 40; SEQ ID NO: 30 and SEQ ID NO: 41; SEQ ID NO: 30 and SEQ ID NO: 42; SEQ ID NO: 30 and SEQ ID NO: 43; SEQ ID NO: 30 and SEQ ID NO: 44; SEQ ID NO: 30 and SEQ ID NO: 45; SEQ ID NO: 30 and SEQ ID NO: 46; SEQ ID NO: 30 and SEQ ID NO: 47; SEQ ID NO: 30 and SEQ ID NO: 48; SEQ ID NO: 30 and SEQ ID NO: 53; SEQ ID NO: 30 and SEQ ID NO: 54; SEQ ID NO: 30 and SEQ ID NO: 55; SEQ ID NO: 30 and SEQ ID NO: 56; SEQ ID NO: 30 and SEQ ID NO: 57; SEQ ID NO: 30 and SEQ ID NO: 58; SEQ ID NO: 30 and SEQ ID NO: 59; SEQ ID NO: 30 and SEQ ID NO: 60; SEQ ID NO: 30 and SEQ ID NO: 61; SEQ ID NO: 30 and SEQ ID NO: 62; SEQ ID NO: 30 and SEQ ID NO: 63; SEQ ID NO: 30 and SEQ ID NO: 64; SEQ ID NO: 30 and SEQ ID NO: 65; SEQ ID NO: 30 and SEQ ID NO: 66; SEQ ID NO: 30 and SEQ ID NO: 67; SEQ ID NO: 30 and SEQ ID NO: 68; SEQ ID NO: 30 and SEQ ID NO: 69; SEQ ID NO: 31 and SEQ ID NO: 32; SEQ ID NO: 31 and SEQ ID NO: 33; SEQ ID NO: 31 and SEQ ID NO: 34; SEQ ID NO: 31 and SEQ ID NO: 35; SEQ ID NO: 31 and SEQ ID NO: 36; SEQ ID NO: 31 and SEQ ID NO: 37; SEQ ID NO: 31 and SEQ ID NO: 38; SEQ ID NO: 31 and SEQ ID NO: 39; SEQ ID NO: 31 and SEQ ID NO: 40; SEQ ID NO: 31 and SEQ ID NO: 41; SEQ ID NO: 31 and SEQ ID NO: 42; SEQ ID NO: 31 and SEQ ID NO: 43; SEQ ID NO: 31 and SEQ ID NO: 44; SEQ ID NO: 31 and SEQ ID NO: 45; SEQ ID NO: 31 and SEQ ID NO: 46; SEQ ID NO: 31 and SEQ ID NO: 47; SEQ ID NO: 31 and SEQ ID NO: 48; SEQ ID NO: 31 and SEQ ID NO: 53; SEQ ID NO: 31 and SEQ ID NO: 54; SEQ ID NO: 31 and SEQ ID NO: 55; SEQ ID NO: 31 and SEQ ID NO: 56; SEQ ID NO: 31 and SEQ ID NO: 57; SEQ ID NO: 31 and SEQ ID NO: 58; SEQ ID NO: 31 and SEQ ID NO: 59; SEQ ID NO: 31 and SEQ ID NO: 60; SEQ ID NO: 31 and SEQ ID NO: 61; SEQ ID NO: 31 and SEQ ID NO: 62; SEQ ID NO: 31 and SEQ ID NO: 63; SEQ ID NO: 31 and SEQ ID NO: 64; SEQ ID NO: 31 and SEQ ID NO: 65; SEQ ID NO: 31 and SEQ ID NO: 66; SEQ ID NO: 31 and SEQ ID NO: 67; SEQ ID NO: 31 and SEQ ID NO: 68; SEQ ID NO: 31 and SEQ ID NO: 69; SEQ ID NO: 32 and SEQ ID NO: 33; SEQ ID NO: 32 and SEQ ID NO: 34; SEQ ID NO: 32 and SEQ ID NO: 35; SEQ ID NO: 32 and SEQ ID NO: 36; SEQ ID NO: 32 and SEQ ID NO: 37; SEQ ID NO: 32 and SEQ ID NO: 38; SEQ ID NO: 32 and SEQ ID NO: 39; SEQ ID NO: 32 and SEQ ID NO: 40; SEQ ID NO: 32 and SEQ ID NO: 41; SEQ ID NO: 32 and SEQ ID NO: 42; SEQ ID NO: 32 and SEQ ID NO: 43; SEQ ID NO: 32 and SEQ ID NO: 44; SEQ ID NO: 32 and SEQ ID NO: 45; SEQ ID NO: 32 and SEQ ID NO: 46; SEQ ID NO: 32 and SEQ ID NO: 47; SEQ ID NO: 32 and SEQ ID NO: 48; SEQ ID NO: 32 and SEQ ID NO: 53; SEQ ID NO: 32 and SEQ ID NO: 54; SEQ ID NO: 32 and SEQ ID NO: 55; SEQ ID NO: 32 and SEQ ID NO: 56; SEQ ID NO: 32 and SEQ ID NO: 57; SEQ ID NO: 32 and SEQ ID NO: 58; SEQ ID NO: 32 and SEQ ID NO: 59; SEQ ID NO: 32 and SEQ ID NO: 60; SEQ ID NO: 32 and SEQ ID NO: 61; SEQ ID NO: 32 and SEQ ID NO: 62; SEQ ID NO: 32 and SEQ ID NO: 63; SEQ ID NO: 32 and SEQ ID NO: 64; SEQ ID NO: 32 and SEQ ID NO: 65; SEQ ID NO: 32 and SEQ ID NO: 66; SEQ ID NO: 32 and SEQ ID NO: 67; SEQ ID NO: 32 and SEQ ID NO: 68; SEQ ID NO: 32 and SEQ ID NO: 69; SEQ ID NO: 33 and SEQ ID NO: 34; SEQ ID NO: 33 and SEQ ID NO: 35; SEQ ID NO: 33 and SEQ ID NO: 36; SEQ ID NO: 33 and SEQ ID NO: 37; SEQ ID NO: 33 and SEQ ID NO: 38; SEQ ID NO: 33 and SEQ ID NO: 39; SEQ ID NO: 33 and SEQ ID NO: 40; SEQ ID NO: 33 and SEQ ID NO: 41; SEQ ID NO: 33 and SEQ ID NO: 42; SEQ ID NO: 33 and SEQ ID NO: 43; SEQ ID NO: 33 and SEQ ID NO: 44; SEQ ID NO: 33 and SEQ ID NO: 45; SEQ ID NO: 33 and SEQ ID NO: 46; SEQ ID NO: 33 and SEQ ID NO: 47; SEQ ID NO: 33 and SEQ ID NO: 48; SEQ ID NO: 33 and SEQ ID NO: 53; SEQ ID NO: 33 and SEQ ID NO: 54; SEQ ID NO: 33 and SEQ ID NO: 55; SEQ ID NO: 33 and SEQ ID NO: 56; SEQ ID NO: 33 and SEQ ID NO: 57; SEQ ID NO: 33 and SEQ ID NO: 58; SEQ ID NO: 33 and SEQ ID NO: 59; SEQ ID NO: 33 and SEQ ID NO: 60; SEQ ID NO: 33 and SEQ ID NO: 61; SEQ ID NO: 33 and SEQ ID NO: 62; SEQ ID NO: 33 and SEQ ID NO: 63; SEQ ID NO: 33 and SEQ ID NO: 64; SEQ ID NO: 33 and SEQ ID NO: 65; SEQ ID NO: 33 and SEQ ID NO: 66; SEQ ID NO: 33 and SEQ ID NO: 67; SEQ ID NO: 33 and SEQ ID NO: 68; SEQ ID NO: 33 and SEQ ID NO: 69; SEQ ID NO: 34 and SEQ ID NO: 35; SEQ ID NO: 34 and SEQ ID NO: 36; SEQ ID NO: 34 and SEQ ID NO: 37; SEQ ID NO: 34 and SEQ ID NO: 38; SEQ ID NO: 34 and SEQ ID NO: 39; SEQ ID NO: 34 and SEQ ID NO: 40; SEQ ID NO: 34 and SEQ ID NO: 41; SEQ ID NO: 34 and SEQ ID NO: 42; SEQ ID NO: 34 and SEQ ID NO: 43; SEQ ID NO: 34 and SEQ ID NO: 44; SEQ ID NO: 34 and SEQ ID NO: 45; SEQ ID NO: 34 and SEQ ID NO: 46; SEQ ID NO: 34 and SEQ ID NO: 47; SEQ ID NO: 34 and SEQ ID NO: 48; SEQ ID NO: 34 and SEQ ID NO: 53; SEQ ID NO: 34 and SEQ ID NO: 54; SEQ ID NO: 34 and SEQ ID NO: 55; SEQ ID NO: 34 and SEQ ID NO: 56; SEQ ID NO: 34 and SEQ ID NO: 57; SEQ ID NO: 34 and SEQ ID NO: 58; SEQ ID NO: 34 and SEQ ID NO: 59; SEQ ID NO: 34 and SEQ ID NO: 60; SEQ ID NO: 34 and SEQ ID NO: 61; SEQ ID NO: 34 and SEQ ID NO: 62; SEQ ID NO: 34 and SEQ ID NO: 63; SEQ ID NO: 34 and SEQ ID NO: 64; SEQ ID NO: 34 and SEQ ID NO: 65; SEQ ID NO: 34 and SEQ ID NO: 66; SEQ ID NO: 34 and SEQ ID NO: 67; SEQ ID NO: 34 and SEQ ID NO: 68; SEQ ID NO: 34 and SEQ ID NO: 69; SEQ ID NO: 35 and SEQ ID NO: 36; SEQ ID NO: 35 and SEQ ID NO: 37; SEQ ID NO: 35 and SEQ ID NO: 38; SEQ ID NO: 35 and SEQ ID NO: 39; SEQ ID NO: 35 and SEQ ID NO: 40; SEQ ID NO: 35 and SEQ ID NO: 41; SEQ ID NO: 35 and SEQ ID NO: 42; SEQ ID NO: 35 and SEQ ID NO: 43; SEQ ID NO: 35 and SEQ ID NO: 44; SEQ ID NO: 35 and SEQ ID NO: 45; SEQ ID NO: 35 and SEQ ID NO: 46; SEQ ID NO: 35 and SEQ ID NO: 47; SEQ ID NO: 35 and SEQ ID NO: 48; SEQ ID NO: 35 and SEQ ID NO: 53; SEQ ID NO: 35 and SEQ ID NO: 54; SEQ ID NO: 35 and SEQ ID NO: 55; SEQ ID NO: 35 and SEQ ID NO: 56; SEQ ID NO: 35 and SEQ ID NO: 57; SEQ ID NO: 35 and SEQ ID NO: 58; SEQ ID NO: 35 and SEQ ID NO: 59; SEQ ID NO: 35 and SEQ ID NO: 60; SEQ ID NO: 35 and SEQ ID NO: 61; SEQ ID NO: 35 and SEQ ID NO: 62; SEQ ID NO: 35 and SEQ ID NO: 63; SEQ ID NO: 35 and SEQ ID NO: 64; SEQ ID NO: 35 and SEQ ID NO: 65; SEQ ID NO: 35 and SEQ ID NO: 66; SEQ ID NO: 35 and SEQ ID NO: 67; SEQ ID NO: 35 and SEQ ID NO: 68; SEQ ID NO: 35 and SEQ ID NO: 69; SEQ ID NO: 36 and SEQ ID NO: 37; SEQ ID NO: 36 and SEQ ID NO: 38; SEQ ID NO: 36 and SEQ ID NO: 39; SEQ ID NO: 36 and SEQ ID NO: 40; SEQ ID NO: 36 and SEQ ID NO: 41; SEQ ID NO: 36 and SEQ ID NO: 42; SEQ ID NO: 36 and SEQ ID NO: 43; SEQ ID NO: 36 and SEQ ID NO: 44; SEQ ID NO: 36 and SEQ ID NO: 45; SEQ ID NO: 36 and SEQ ID NO: 46; SEQ ID NO: 36 and SEQ ID NO: 47; SEQ ID NO: 36 and SEQ ID NO: 48; SEQ ID NO: 36 and SEQ ID NO: 53; SEQ ID NO: 36 and SEQ ID NO: 54; SEQ ID NO: 36 and SEQ ID NO: 55; SEQ ID NO: 36 and SEQ ID NO: 56; SEQ ID NO: 36 and SEQ ID NO: 57; SEQ ID NO: 36 and SEQ ID NO: 58; SEQ ID NO: 36 and SEQ ID NO: 59; SEQ ID NO: 36 and SEQ ID NO: 60; SEQ ID NO: 36 and SEQ ID NO: 61; SEQ ID NO: 36 and SEQ ID NO: 62; SEQ ID NO: 36 and SEQ ID NO: 63; SEQ ID NO: 36 and SEQ ID NO: 64; SEQ ID NO: 36 and SEQ ID NO: 65; SEQ ID NO: 36 and SEQ ID NO: 66; SEQ ID NO: 36 and SEQ ID NO: 67; SEQ ID NO: 36 and SEQ ID NO: 68; SEQ ID NO: 36 and SEQ ID NO: 69; SEQ ID NO: 37 and SEQ ID NO: 38; SEQ ID NO: 37 and SEQ ID NO: 39; SEQ ID NO: 37 and SEQ ID NO: 40; SEQ ID NO: 37 and SEQ ID NO: 41; SEQ ID NO: 37 and SEQ ID NO: 42; SEQ ID NO: 37 and SEQ ID NO: 43; SEQ ID NO: 37 and SEQ ID NO: 44; SEQ ID NO: 37 and SEQ ID NO: 45; SEQ ID NO: 37 and SEQ ID NO: 46; SEQ ID NO: 37 and SEQ ID NO: 47; SEQ ID NO: 37 and SEQ ID NO: 48; SEQ ID NO: 37 and SEQ ID NO: 53; SEQ ID NO: 37 and SEQ ID NO: 54; SEQ ID NO: 37 and SEQ ID NO: 55; SEQ ID NO: 37 and SEQ ID NO: 56; SEQ ID NO: 37 and SEQ ID NO: 57; SEQ ID NO: 37 and SEQ ID NO: 58; SEQ ID NO: 37 and SEQ ID NO: 59; SEQ ID NO: 37 and SEQ ID NO: 60; SEQ ID NO: 37 and SEQ ID NO: 61; SEQ ID NO: 37 and SEQ ID NO: 62; SEQ ID NO: 37 and SEQ ID NO: 63; SEQ ID NO: 37 and SEQ ID NO: 64; SEQ ID NO: 37 and SEQ ID NO: 65; SEQ ID NO: 37 and SEQ ID NO: 66; SEQ ID NO: 37 and SEQ ID NO: 67; SEQ ID NO: 37 and SEQ ID NO: 68; SEQ ID NO: 37 and SEQ ID NO: 69; SEQ ID NO: 38 and SEQ ID NO: 39; SEQ ID NO: 38 and SEQ ID NO: 40; SEQ ID NO: 38 and SEQ ID NO: 41; SEQ ID NO: 38 and SEQ ID NO: 42; SEQ ID NO: 38 and SEQ ID NO: 43; SEQ ID NO: 38 and SEQ ID NO: 44; SEQ ID NO: 38 and SEQ ID NO: 45; SEQ ID NO: 38 and SEQ ID NO: 46; SEQ ID NO: 38 and SEQ ID NO: 47; SEQ ID NO: 38 and SEQ ID NO: 48; SEQ ID NO: 38 and SEQ ID NO: 53; SEQ ID NO: 38 and SEQ ID NO: 54; SEQ ID NO: 38 and SEQ ID NO: 55; SEQ ID NO: 38 and SEQ ID NO: 56; SEQ ID NO: 38 and SEQ ID NO: 57; SEQ ID NO: 38 and SEQ ID NO: 58; SEQ ID NO: 38 and SEQ ID NO: 59; SEQ ID NO: 38 and SEQ ID NO: 60; SEQ ID NO: 38 and SEQ ID NO: 61; SEQ ID NO: 38 and SEQ ID NO: 62; SEQ ID NO: 38 and SEQ ID NO: 63; SEQ ID NO: 38 and SEQ ID NO: 64; SEQ ID NO: 38 and SEQ ID NO: 65; SEQ ID NO: 38 and SEQ ID NO: 66; SEQ ID NO: 38 and SEQ ID NO: 67; SEQ ID NO: 38 and SEQ ID NO: 68; SEQ ID NO: 38 and SEQ ID NO: 69; SEQ ID NO: 39 and SEQ ID NO: 40; SEQ ID NO: 39 and SEQ ID NO: 41; SEQ ID NO: 39 and SEQ ID NO: 42; SEQ ID NO: 39 and SEQ ID NO: 43; SEQ ID NO: 39 and SEQ ID NO: 44; SEQ ID NO: 39 and SEQ ID NO: 45; SEQ ID NO: 39 and SEQ ID NO: 46; SEQ ID NO: 39 and SEQ ID NO: 47; SEQ ID NO: 39 and SEQ ID NO: 48; SEQ ID NO: 39 and SEQ ID NO: 53; SEQ ID NO: 39 and SEQ ID NO: 54; SEQ ID NO: 39 and SEQ ID NO: 55; SEQ ID NO: 39 and SEQ ID NO: 56; SEQ ID NO: 39 and SEQ ID NO: 57; SEQ ID NO: 39 and SEQ ID NO: 58; SEQ ID NO: 39 and SEQ ID NO: 59; SEQ ID NO: 39 and SEQ ID NO: 60; SEQ ID NO: 39 and SEQ ID NO: 61; SEQ ID NO: 39 and SEQ ID NO: 62; SEQ ID NO: 39 and SEQ ID NO: 63; SEQ ID NO: 39 and SEQ ID NO: 64; SEQ ID NO: 39 and SEQ ID NO: 65; SEQ ID NO: 39 and SEQ ID NO: 66; SEQ ID NO: 39 and SEQ ID NO: 67; SEQ ID NO: 39 and SEQ ID NO: 68; SEQ ID NO: 39 and SEQ ID NO: 69; SEQ ID NO: 40 and SEQ ID NO: 41; SEQ ID NO: 40 and SEQ ID NO: 42; SEQ ID NO: 40 and SEQ ID NO: 43; SEQ ID NO: 40 and SEQ ID NO: 44; SEQ ID NO: 40 and SEQ ID NO: 45; SEQ ID NO: 40 and SEQ ID NO: 46; SEQ ID NO: 40 and SEQ ID NO: 47; SEQ ID NO: 40 and SEQ ID NO: 48; SEQ ID NO: 40 and SEQ ID NO: 53; SEQ ID NO: 40 and SEQ ID NO: 54; SEQ ID NO: 40 and SEQ ID NO: 55; SEQ ID NO: 40 and SEQ ID NO: 56; SEQ ID NO: 40 and SEQ ID NO: 57; SEQ ID NO: 40 and SEQ ID NO: 58; SEQ ID NO: 40 and SEQ ID NO: 59; SEQ ID NO: 40 and SEQ ID NO: 60; SEQ ID NO: 40 and SEQ ID NO: 61; SEQ ID NO: 40 and SEQ ID NO: 62; SEQ ID NO: 40 and SEQ ID NO: 63; SEQ ID NO: 40 and SEQ ID NO: 64; SEQ ID NO: 40 and SEQ ID NO: 65; SEQ ID NO: 40 and SEQ ID NO: 66; SEQ ID NO: 40 and SEQ ID NO: 67; SEQ ID NO: 40 and SEQ ID NO: 68; SEQ ID NO: 40 and SEQ ID NO: 69; SEQ ID NO: 41 and SEQ ID NO: 42; SEQ ID NO: 41 and SEQ ID NO: 43; SEQ ID NO: 41 and SEQ ID NO: 44; SEQ ID NO: 41 and SEQ ID NO: 45; SEQ ID NO: 41 and SEQ ID NO: 46; SEQ ID NO: 41 and SEQ ID NO: 47; SEQ ID NO: 41 and SEQ ID NO: 48; SEQ ID NO: 41 and SEQ ID NO: 53; SEQ ID NO: 41 and SEQ ID NO: 54; SEQ ID NO: 41 and SEQ ID NO: 55; SEQ ID NO: 41 and SEQ ID NO: 56; SEQ ID NO: 41 and SEQ ID NO: 57; SEQ ID NO: 41 and SEQ ID NO: 58; SEQ ID NO: 41 and SEQ ID NO: 59; SEQ ID NO: 41 and SEQ ID NO: 60; SEQ ID NO: 41 and SEQ ID NO: 61; SEQ ID NO: 41 and SEQ ID NO: 62; SEQ ID NO: 41 and SEQ ID NO: 63; SEQ ID NO: 41 and SEQ ID NO: 64; SEQ ID NO: 41 and SEQ ID NO: 65; SEQ ID NO: 41 and SEQ ID NO: 66; SEQ ID NO: 41 and SEQ ID NO: 67; SEQ ID NO: 41 and SEQ ID NO: 68; SEQ ID NO: 41 and SEQ ID NO: 69; SEQ ID NO: 42 and SEQ ID NO: 43; SEQ ID NO: 42 and SEQ ID NO: 44; SEQ ID NO: 42 and SEQ ID NO: 45; SEQ ID NO: 42 and SEQ ID NO: 46; SEQ ID NO: 42 and SEQ ID NO: 47; SEQ ID NO: 42 and SEQ ID NO: 48; SEQ ID NO: 42 and SEQ ID NO: 53; SEQ ID NO: 42 and SEQ ID NO: 54; SEQ ID NO: 42 and SEQ ID NO: 55; SEQ ID NO: 42 and SEQ ID NO: 56; SEQ ID NO: 42 and SEQ ID NO: 57; SEQ ID NO: 42 and SEQ ID NO: 58; SEQ ID NO: 42 and SEQ ID NO: 59; SEQ ID NO: 42 and SEQ ID NO: 60; SEQ ID NO: 42 and SEQ ID NO: 61; SEQ ID NO: 42 and SEQ ID NO: 62; SEQ ID NO: 42 and SEQ ID NO: 63; SEQ ID NO: 42 and SEQ ID NO: 64; SEQ ID NO: 42 and SEQ ID NO: 65; SEQ ID NO: 42 and SEQ ID NO: 66; SEQ ID NO: 42 and SEQ ID NO: 67; SEQ ID NO: 42 and SEQ ID NO: 68; SEQ ID NO: 42 and SEQ ID NO: 69; SEQ ID NO: 43 and SEQ ID NO: 44; SEQ ID NO: 43 and SEQ ID NO: 45; SEQ ID NO: 43 and SEQ ID NO: 46; SEQ ID NO: 43 and SEQ ID NO: 47; SEQ ID NO: 43 and SEQ ID NO: 48; SEQ ID NO: 43 and SEQ ID NO: 53; SEQ ID NO: 43 and SEQ ID NO: 54; SEQ ID NO: 43 and SEQ ID NO: 55; SEQ ID NO: 43 and SEQ ID NO: 56; SEQ ID NO: 43 and SEQ ID NO: 57; SEQ ID NO: 43 and SEQ ID NO: 58; SEQ ID NO: 43 and SEQ ID NO: 59; SEQ ID NO: 43 and SEQ ID NO: 60; SEQ ID NO: 43 and SEQ ID NO: 61; SEQ ID NO: 43 and SEQ ID NO: 62; SEQ ID NO: 43 and SEQ ID NO: 63; SEQ ID NO: 43 and SEQ ID NO: 64; SEQ ID NO: 43 and SEQ ID NO: 65; SEQ ID NO: 43 and SEQ ID NO: 66; SEQ ID NO: 43 and SEQ ID NO: 67; SEQ ID NO: 43 and SEQ ID NO: 68; SEQ ID NO: 43 and SEQ ID NO: 69; SEQ ID NO: 44 and SEQ ID NO: 45; SEQ ID NO: 44 and SEQ ID NO: 46; SEQ ID NO: 44 and SEQ ID NO: 47; SEQ ID NO: 44 and SEQ ID NO: 48; SEQ ID NO: 44 and SEQ ID NO: 53; SEQ ID NO: 44 and SEQ ID NO: 54; SEQ ID NO: 44 and SEQ ID NO: 55; SEQ ID NO: 44 and SEQ ID NO: 56; SEQ ID NO: 44 and SEQ ID NO: 57; SEQ ID NO: 44 and SEQ ID NO: 58; SEQ ID NO: 44 and SEQ ID NO: 59; SEQ ID NO: 44 and SEQ ID NO: 60; SEQ ID NO: 44 and SEQ ID NO: 61; SEQ ID NO: 44 and SEQ ID NO: 62; SEQ ID NO: 44 and SEQ ID NO: 63; SEQ ID NO: 44 and SEQ ID NO: 64; SEQ ID NO: 44 and SEQ ID NO: 65; SEQ ID NO: 44 and SEQ ID NO: 66; SEQ ID NO: 44 and SEQ ID NO: 67; SEQ ID NO: 44 and SEQ ID NO: 68; SEQ ID NO: 44 and SEQ ID NO: 69; SEQ ID NO: 45 and SEQ ID NO: 46; SEQ ID NO: 45 and SEQ ID NO: 47; SEQ ID NO: 45 and SEQ ID NO: 48; SEQ ID NO: 45 and SEQ ID NO: 53; SEQ ID NO: 45 and SEQ ID NO: 54; SEQ ID NO: 45 and SEQ ID NO: 55; SEQ ID NO: 45 and SEQ ID NO: 56; SEQ ID NO: 45 and SEQ ID NO: 57; SEQ ID NO: 45 and SEQ ID NO: 58; SEQ ID NO: 45 and SEQ ID NO: 59; SEQ ID NO: 45 and SEQ ID NO: 60; SEQ ID NO: 45 and SEQ ID NO: 61; SEQ ID NO: 45 and SEQ ID NO: 62; SEQ ID NO: 45 and SEQ ID NO: 63; SEQ ID NO: 45 and SEQ ID NO: 64; SEQ ID NO: 45 and SEQ ID NO: 65; SEQ ID NO: 45 and SEQ ID NO: 66; SEQ ID NO: 45 and SEQ ID NO: 67; SEQ ID NO: 45 and SEQ ID NO: 68; SEQ ID NO: 45 and SEQ ID NO: 69; SEQ ID NO: 46 and SEQ ID NO: 47; SEQ ID NO: 46 and SEQ ID NO: 48; SEQ ID NO: 46 and SEQ ID NO: 53; SEQ ID NO: 46 and SEQ ID NO: 54; SEQ ID NO: 46 and SEQ ID NO: 55; SEQ ID NO: 46 and SEQ ID NO: 56; SEQ ID NO: 46 and SEQ ID NO: 57; SEQ ID NO: 46 and SEQ ID NO: 58; SEQ ID NO: 46 and SEQ ID NO: 59; SEQ ID NO: 46 and SEQ ID NO: 60; SEQ ID NO: 46 and SEQ ID NO: 61; SEQ ID NO: 46 and SEQ ID NO: 62; SEQ ID NO: 46 and SEQ ID NO: 63; SEQ ID NO: 46 and SEQ ID NO: 64; SEQ ID NO: 46 and SEQ ID NO: 65; SEQ ID NO: 46 and SEQ ID NO: 66; SEQ ID NO: 46 and SEQ ID NO: 67; SEQ ID NO: 46 and SEQ ID NO: 68; SEQ ID NO: 46 and SEQ ID NO: 69; SEQ ID NO: 47 and SEQ ID NO: 48; SEQ ID NO: 47 and SEQ ID NO: 53; SEQ ID NO: 47 and SEQ ID NO: 54; SEQ ID NO: 47 and SEQ ID NO: 55; SEQ ID NO: 47 and SEQ ID NO: 56; SEQ ID NO: 47 and SEQ ID NO: 57; SEQ ID NO: 47 and SEQ ID NO: 58; SEQ ID NO: 47 and SEQ ID NO: 59; SEQ ID NO: 47 and SEQ ID NO: 60; SEQ ID NO: 47 and SEQ ID NO: 61; SEQ ID NO: 47 and SEQ ID NO: 62; SEQ ID NO: 47 and SEQ ID NO: 63; SEQ ID NO: 47 and SEQ ID NO: 64; SEQ ID NO: 47 and SEQ ID NO: 65; SEQ ID NO: 47 and SEQ ID NO: 66; SEQ ID NO: 47 and SEQ ID NO: 67; SEQ ID NO: 47 and SEQ ID NO: 68; SEQ ID NO: 47 and SEQ ID NO: 69; SEQ ID NO: 48 and SEQ ID NO: 53; SEQ ID NO: 48 and SEQ ID NO: 54; SEQ ID NO: 48 and SEQ ID NO: 55; SEQ ID NO: 48 and SEQ ID NO: 56; SEQ ID NO: 48 and SEQ ID NO: 57; SEQ ID NO: 48 and SEQ ID NO: 58; SEQ ID NO: 48 and SEQ ID NO: 59; SEQ ID NO: 48 and SEQ ID NO: 60; SEQ ID NO: 48 and SEQ ID NO: 61; SEQ ID NO: 48 and SEQ ID NO: 62; SEQ ID NO: 48 and SEQ ID NO: 63; SEQ ID NO: 48 and SEQ ID NO: 64; SEQ ID NO: 48 and SEQ ID NO: 65; SEQ ID NO: 48 and SEQ ID NO: 66; SEQ ID NO: 48 and SEQ ID NO: 67; SEQ ID NO: 48 and SEQ ID NO: 68; SEQ ID NO: 48 and SEQ ID NO: 69; SEQ ID NO: 53 and SEQ ID NO: 54; SEQ ID NO: 53 and SEQ ID NO: 55; SEQ ID NO: 53 and SEQ ID NO: 56; SEQ ID NO: 53 and SEQ ID NO: 57; SEQ ID NO: 53 and SEQ ID NO: 58; SEQ ID NO: 53 and SEQ ID NO: 59; SEQ ID NO: 53 and SEQ ID NO: 60; SEQ ID NO: 53 and SEQ ID NO: 61; SEQ ID NO: 53 and SEQ ID NO: 62; SEQ ID NO: 53 and SEQ ID NO: 63; SEQ ID NO: 53 and SEQ ID NO: 64; SEQ ID NO: 53 and SEQ ID NO: 65; SEQ ID NO: 53 and SEQ ID NO: 66; SEQ ID NO: 53 and SEQ ID NO: 67; SEQ ID NO: 53 and SEQ ID NO: 68; SEQ ID NO: 53 and SEQ ID NO: 69; SEQ ID NO: 54 and SEQ ID NO: 55; SEQ ID NO: 54 and SEQ ID NO: 56; SEQ ID NO: 54 and SEQ ID NO: 57; SEQ ID NO: 54 and SEQ ID NO: 58; SEQ ID NO: 54 and SEQ ID NO: 59; SEQ ID NO: 54 and SEQ ID NO: 60; SEQ ID NO: 54 and SEQ ID NO: 61; SEQ ID NO: 54 and SEQ ID NO: 62; SEQ ID NO: 54 and SEQ ID NO: 63; SEQ ID NO: 54 and SEQ ID NO: 64; SEQ ID NO: 54 and SEQ ID NO: 65; SEQ ID NO: 54 and SEQ ID NO: 66; SEQ ID NO: 54 and SEQ ID NO: 67; SEQ ID NO: 54 and SEQ ID NO: 68; SEQ ID NO: 54 and SEQ ID NO: 69; SEQ ID NO: 55 and SEQ ID NO: 56; SEQ ID NO: 55 and SEQ ID NO: 57; SEQ ID NO: 55 and SEQ ID NO: 58; SEQ ID NO: 55 and SEQ ID NO: 59; SEQ ID NO: 55 and SEQ ID NO: 60; SEQ ID NO: 55 and SEQ ID NO: 61; SEQ ID NO: 55 and SEQ ID NO: 62; SEQ ID NO: 55 and SEQ ID NO: 63; SEQ ID NO: 55 and SEQ ID NO: 64; SEQ ID NO: 55 and SEQ ID NO: 65; SEQ ID NO: 55 and SEQ ID NO: 66; SEQ ID NO: 55 and SEQ ID NO: 67; SEQ ID NO: 55 and SEQ ID NO: 68; SEQ ID NO: 55 and SEQ ID NO: 69; SEQ ID NO: 56 and SEQ ID NO: 57; SEQ ID NO: 56 and SEQ ID NO: 58; SEQ ID NO: 56 and SEQ ID NO: 59; SEQ ID NO: 56 and SEQ ID NO: 60; SEQ ID NO: 56 and SEQ ID NO: 61; SEQ ID NO: 56 and SEQ ID NO: 62; SEQ ID NO: 56 and SEQ ID NO: 63; SEQ ID NO: 56 and SEQ ID NO: 64; SEQ ID NO: 56 and SEQ ID NO: 65; SEQ ID NO: 56 and SEQ ID NO: 66; SEQ ID NO: 56 and SEQ ID NO: 67; SEQ ID NO: 56 and SEQ ID NO: 68; SEQ ID NO: 56 and SEQ ID NO: 69; SEQ ID NO: 57 and SEQ ID NO: 58; SEQ ID NO: 57 and SEQ ID NO: 59; SEQ ID NO: 57 and SEQ ID NO: 60; SEQ ID NO: 57 and SEQ ID NO: 61; SEQ ID NO: 57 and SEQ ID NO: 62; SEQ ID NO: 57 and SEQ ID NO: 63; SEQ ID NO: 57 and SEQ ID NO: 64; SEQ ID NO: 57 and SEQ ID NO: 65; SEQ ID NO: 57 and SEQ ID NO: 66; SEQ ID NO: 57 and SEQ ID NO: 67; SEQ ID NO: 57 and SEQ ID NO: 68; SEQ ID NO: 57 and SEQ ID NO: 69; SEQ ID NO: 58 and SEQ ID NO: 59; SEQ ID NO: 58 and SEQ ID NO: 60; SEQ ID NO: 58 and SEQ ID NO: 61; SEQ ID NO: 58 and SEQ ID NO: 62; SEQ ID NO: 58 and SEQ ID NO: 63; SEQ ID NO: 58 and SEQ ID NO: 64; SEQ ID NO: 58 and SEQ ID NO: 65; SEQ ID NO: 58 and SEQ ID NO: 66; SEQ ID NO: 58 and SEQ ID NO: 67; SEQ ID NO: 58 and SEQ ID NO: 68; SEQ ID NO: 58 and SEQ ID NO: 69; SEQ ID NO: 59 and SEQ ID NO: 60; SEQ ID NO: 59 and SEQ ID NO: 61; SEQ ID NO: 59 and SEQ ID NO: 62; SEQ ID NO: 59 and SEQ ID NO: 63; SEQ ID NO: 59 and SEQ ID NO: 64; SEQ ID NO: 59 and SEQ ID NO: 65; SEQ ID NO: 59 and SEQ ID NO: 66; SEQ ID NO: 59 and SEQ ID NO: 67; SEQ ID NO: 59 and SEQ ID NO: 68; SEQ ID NO: 59 and SEQ ID NO: 69; SEQ ID NO: 60 and SEQ ID NO: 61; SEQ ID NO: 60 and SEQ ID NO: 62; SEQ ID NO: 60 and SEQ ID NO: 63; SEQ ID NO: 60 and SEQ ID NO: 64; SEQ ID NO: 60 and SEQ ID NO: 65; SEQ ID NO: 60 and SEQ ID NO: 66; SEQ ID NO: 60 and SEQ ID NO: 67; SEQ ID NO: 60 and SEQ ID NO: 68; SEQ ID NO: 60 and SEQ ID NO: 69; SEQ ID NO: 61 and SEQ ID NO: 62; SEQ ID NO: 61 and SEQ ID NO: 63; SEQ ID NO: 61 and SEQ ID NO: 64; SEQ ID NO: 61 and SEQ ID NO: 65; SEQ ID NO: 61 and SEQ ID NO: 66; SEQ ID NO: 61 and SEQ ID NO: 67; SEQ ID NO: 61 and SEQ ID NO: 68; SEQ ID NO: 61 and SEQ ID NO: 69; SEQ ID NO: 62 and SEQ ID NO: 63; SEQ ID NO: 62 and SEQ ID NO: 64; SEQ ID NO: 62 and SEQ ID NO: 65; SEQ ID NO: 62 and SEQ ID NO: 66; SEQ ID NO: 62 and SEQ ID NO: 67; SEQ ID NO: 62 and SEQ ID NO: 68; SEQ ID NO: 62 and SEQ ID NO: 69; SEQ ID NO: 63 and SEQ ID NO: 64; SEQ ID NO: 63 and SEQ ID NO: 65; SEQ ID NO: 63 and SEQ ID NO: 66; SEQ ID NO: 63 and SEQ ID NO: 67; SEQ ID NO: 63 and SEQ ID NO: 68; SEQ ID NO: 63 and SEQ ID NO: 69; SEQ ID NO: 64 and SEQ ID NO: 65; SEQ ID NO: 64 and SEQ ID NO: 66; SEQ ID NO: 64 and SEQ ID NO: 67; SEQ ID NO: 64 and SEQ ID NO: 68; SEQ ID NO: 64 and SEQ ID NO: 69; SEQ ID NO: 65 and SEQ ID NO: 66; SEQ ID NO: 65 and SEQ ID NO: 67; SEQ ID NO: 65 and SEQ ID NO: 68; SEQ ID NO: 65 and SEQ ID NO: 69; SEQ ID NO: 66 and SEQ ID NO: 67; SEQ ID NO: 66 and SEQ ID NO: 68; SEQ ID NO: 66 and SEQ ID NO: 69; SEQ ID NO: 67 and SEQ ID NO: 68; SEQ ID NO: 67 and SEQ ID NO: 69; SEQ ID NO: 68 and SEQ ID NO: 69; SEQ ID NO: 71 and SEQ ID NO: 72; SEQ ID NO: 71 and SEQ ID NO: 73; SEQ ID NO: 71 and SEQ ID NO: 74; SEQ ID NO: 71 and SEQ ID NO: 75; SEQ ID NO: 71 and SEQ ID NO: 76; SEQ ID NO: 71 and SEQ ID NO: 77; SEQ ID NO: 71 and SEQ ID NO: 78; SEQ ID NO: 71 and SEQ ID NO: 84; SEQ ID NO: 71 and SEQ ID NO: 85; SEQ ID NO: 71 and SEQ ID NO: 86; SEQ ID NO: 71 and SEQ ID NO: 87; SEQ ID NO: 71 and SEQ ID NO: 88; SEQ ID NO: 71 and SEQ ID NO: 89; SEQ ID NO: 71 and SEQ ID NO: 90; SEQ ID NO: 71 and SEQ ID NO: 91; SEQ ID NO: 71 and SEQ ID NO: 92; SEQ ID NO: 71 and SEQ ID NO: 93; SEQ ID NO: 71 and SEQ ID NO: 94; SEQ ID NO: 71 and SEQ ID NO: 95; SEQ ID NO: 71 and SEQ ID NO: 96; SEQ ID NO: 71 and SEQ ID NO: 97; SEQ ID NO: 71 and SEQ ID NO: 98; SEQ ID NO: 71 and SEQ ID NO: 99; SEQ ID NO: 71 and SEQ ID NO: 100; SEQ ID NO: 71 and SEQ ID NO: 101; SEQ ID NO: 71 and SEQ ID NO: 102; SEQ ID NO: 72 and SEQ ID NO: 73; SEQ ID NO: 72 and SEQ ID NO: 74; SEQ ID NO: 72 and SEQ ID NO: 75; SEQ ID NO: 72 and SEQ ID NO: 76; SEQ ID NO: 72 and SEQ ID NO: 77; SEQ ID NO: 72 and SEQ ID NO: 78; SEQ ID NO: 72 and SEQ ID NO: 84; SEQ ID NO: 72 and SEQ ID NO: 85; SEQ ID NO: 72 and SEQ ID NO: 86; SEQ ID NO: 72 and SEQ ID NO: 87; SEQ ID NO: 72 and SEQ ID NO: 88; SEQ ID NO: 72 and SEQ ID NO: 89; SEQ ID NO: 72 and SEQ ID NO: 90; SEQ ID NO: 72 and SEQ ID NO: 91; SEQ ID NO: 72 and SEQ ID NO: 92; SEQ ID NO: 72 and SEQ ID NO: 93; SEQ ID NO: 72 and SEQ ID NO: 94; SEQ ID NO: 72 and SEQ ID NO: 95; SEQ ID NO: 72 and SEQ ID NO: 96; SEQ ID NO: 72 and SEQ ID NO: 97; SEQ ID NO: 72 and SEQ ID NO: 98; SEQ ID NO: 72 and SEQ ID NO: 99; SEQ ID NO: 72 and SEQ ID NO: 100; SEQ ID NO: 72 and SEQ ID NO: 101; SEQ ID NO: 72 and SEQ ID NO: 102; SEQ ID NO: 73 and SEQ ID NO: 74; SEQ ID NO: 73 and SEQ ID NO: 75; SEQ ID NO: 73 and SEQ ID NO: 76; SEQ ID NO: 73 and SEQ ID NO: 77; SEQ ID NO: 73 and SEQ ID NO: 78; SEQ ID NO: 73 and SEQ ID NO: 84; SEQ ID NO: 73 and SEQ ID NO: 85; SEQ ID NO: 73 and SEQ ID NO: 86; SEQ ID NO: 73 and SEQ ID NO: 87; SEQ ID NO: 73 and SEQ ID NO: 88; SEQ ID NO: 73 and SEQ ID NO: 89; SEQ ID NO: 73 and SEQ ID NO: 90; SEQ ID NO: 73 and SEQ ID NO: 91; SEQ ID NO: 73 and SEQ ID NO: 92; SEQ ID NO: 73 and SEQ ID NO: 93; SEQ ID NO: 73 and SEQ ID NO: 94; SEQ ID NO: 73 and SEQ ID NO: 95; SEQ ID NO: 73 and SEQ ID NO: 96; SEQ ID NO: 73 and SEQ ID NO: 97; SEQ ID NO: 73 and SEQ ID NO: 98; SEQ ID NO: 73 and SEQ ID NO: 99; SEQ ID NO: 73 and SEQ ID NO: 100; SEQ ID NO: 73 and SEQ ID NO: 101; SEQ ID NO: 73 and SEQ ID NO: 102; SEQ ID NO: 74 and SEQ ID NO: 75; SEQ ID NO: 74 and SEQ ID NO: 76; SEQ ID NO: 74 and SEQ ID NO: 77; SEQ ID NO: 74 and SEQ ID NO: 78; SEQ ID NO: 74 and SEQ ID NO: 84; SEQ ID NO: 74 and SEQ ID NO: 85; SEQ ID NO: 74 and SEQ ID NO: 86; SEQ ID NO: 74 and SEQ ID NO: 87; SEQ ID NO: 74 and SEQ ID NO: 88; SEQ ID NO: 74 and SEQ ID NO: 89; SEQ ID NO: 74 and SEQ ID NO: 90; SEQ ID NO: 74 and SEQ ID NO: 91; SEQ ID NO: 74 and SEQ ID NO: 92; SEQ ID NO: 74 and SEQ ID NO: 93; SEQ ID NO: 74 and SEQ ID NO: 94; SEQ ID NO: 74 and SEQ ID NO: 95; SEQ ID NO: 74 and SEQ ID NO: 96; SEQ ID NO: 74 and SEQ ID NO: 97; SEQ ID NO: 74 and SEQ ID NO: 98; SEQ ID NO: 74 and SEQ ID NO: 99; SEQ ID NO: 74 and SEQ ID NO: 100; SEQ ID NO: 74 and SEQ ID NO: 101; SEQ ID NO: 74 and SEQ ID NO: 102; SEQ ID NO: 75 and SEQ ID NO: 76; SEQ ID NO: 75 and SEQ ID NO: 77; SEQ ID NO: 75 and SEQ ID NO: 78; SEQ ID NO: 75 and SEQ ID NO: 84; SEQ ID NO: 75 and SEQ ID NO: 85; SEQ ID NO: 75 and SEQ ID NO: 86; SEQ ID NO: 75 and SEQ ID NO: 87; SEQ ID NO: 75 and SEQ ID NO: 88; SEQ ID NO: 75 and SEQ ID NO: 89; SEQ ID NO: 75 and SEQ ID NO: 90; SEQ ID NO: 75 and SEQ ID NO: 91; SEQ ID NO: 75 and SEQ ID NO: 92; SEQ ID NO: 75 and SEQ ID NO: 93; SEQ ID NO: 75 and SEQ ID NO: 94; SEQ ID NO: 75 and SEQ ID NO: 95; SEQ ID NO: 75 and SEQ ID NO: 96; SEQ ID NO: 75 and SEQ ID NO: 97; SEQ ID NO: 75 and SEQ ID NO: 98; SEQ ID NO: 75 and SEQ ID NO: 99; SEQ ID NO: 75 and SEQ ID NO: 100; SEQ ID NO: 75 and SEQ ID NO: 101; SEQ ID NO: 75 and SEQ ID NO: 102; SEQ ID NO: 76 and SEQ ID NO: 77; SEQ ID NO: 76 and SEQ ID NO: 78; SEQ ID NO: 76 and SEQ ID NO: 84; SEQ ID NO: 76 and SEQ ID NO: 85; SEQ ID NO: 76 and SEQ ID NO: 86; SEQ ID NO: 76 and SEQ ID NO: 87; SEQ ID NO: 76 and SEQ ID NO: 88; SEQ ID NO: 76 and SEQ ID NO: 89; SEQ ID NO: 76 and SEQ ID NO: 90; SEQ ID NO: 76 and SEQ ID NO: 91; SEQ ID NO: 76 and SEQ ID NO: 92; SEQ ID NO: 76 and SEQ ID NO: 93; SEQ ID NO: 76 and SEQ ID NO: 94; SEQ ID NO: 76 and SEQ ID NO: 95; SEQ ID NO: 76 and SEQ ID NO: 96; SEQ ID NO: 76 and SEQ ID NO: 97; SEQ ID NO: 76 and SEQ ID NO: 98; SEQ ID NO: 76 and SEQ ID NO: 99; SEQ ID NO: 76 and SEQ ID NO: 100; SEQ ID NO: 76 and SEQ ID NO: 101; SEQ ID NO: 76 and SEQ ID NO: 102; SEQ ID NO: 77 and SEQ ID NO: 78; SEQ ID NO: 77 and SEQ ID NO: 84; SEQ ID NO: 77 and SEQ ID NO: 85; SEQ ID NO: 77 and SEQ ID NO: 86; SEQ ID NO: 77 and SEQ ID NO: 87; SEQ ID NO: 77 and SEQ ID NO: 88; SEQ ID NO: 77 and SEQ ID NO: 89; SEQ ID NO: 77 and SEQ ID NO: 90; SEQ ID NO: 77 and SEQ ID NO: 91; SEQ ID NO: 77 and SEQ ID NO: 92; SEQ ID NO: 77 and SEQ ID NO: 93; SEQ ID NO: 77 and SEQ ID NO: 94; SEQ ID NO: 77 and SEQ ID NO: 95; SEQ ID NO: 77 and SEQ ID NO: 96; SEQ ID NO: 77 and SEQ ID NO: 97; SEQ ID NO: 77 and SEQ ID NO: 98; SEQ ID NO: 77 and SEQ ID NO: 99; SEQ ID NO: 77 and SEQ ID NO: 100; SEQ ID NO: 77 and SEQ ID NO: 101; SEQ ID NO: 77 and SEQ ID NO: 102; SEQ ID NO: 78 and SEQ ID NO: 84; SEQ ID NO: 78 and SEQ ID NO: 85; SEQ ID NO: 78 and SEQ ID NO: 86; SEQ ID NO: 78 and SEQ ID NO: 87; SEQ ID NO: 78 and SEQ ID NO: 88; SEQ ID NO: 78 and SEQ ID NO: 89; SEQ ID NO: 78 and SEQ ID NO: 90; SEQ ID NO: 78 and SEQ ID NO: 91; SEQ ID NO: 78 and SEQ ID NO: 92; SEQ ID NO: 78 and SEQ ID NO: 93; SEQ ID NO: 78 and SEQ ID NO: 94; SEQ ID NO: 78 and SEQ ID NO: 95; SEQ ID NO: 78 and SEQ ID NO: 96; SEQ ID NO: 78 and SEQ ID NO: 97; SEQ ID NO: 78 and SEQ ID NO: 98; SEQ ID NO: 78 and SEQ ID NO: 99; SEQ ID NO: 78 and SEQ ID NO: 100; SEQ ID NO: 78 and SEQ ID NO: 101; SEQ ID NO: 78 and SEQ ID NO: 102; SEQ ID NO: 84 and SEQ ID NO: 85; SEQ ID NO: 84 and SEQ ID NO: 86; SEQ ID NO: 84 and SEQ ID NO: 87; SEQ ID NO: 84 and SEQ ID NO: 88; SEQ ID NO: 84 and SEQ ID NO: 89; SEQ ID NO: 84 and SEQ ID NO: 90; SEQ ID NO: 84 and SEQ ID NO: 91; SEQ ID NO: 84 and SEQ ID NO: 92; SEQ ID NO: 84 and SEQ ID NO: 93; SEQ ID NO: 84 and SEQ ID NO: 94; SEQ ID NO: 84 and SEQ ID NO: 95; SEQ ID NO: 84 and SEQ ID NO: 96; SEQ ID NO: 84 and SEQ ID NO: 97; SEQ ID NO: 84 and SEQ ID NO: 98; SEQ ID NO: 84 and SEQ ID NO: 99; SEQ ID NO: 84 and SEQ ID NO: 100; SEQ ID NO: 84 and SEQ ID NO: 101; SEQ ID NO: 84 and SEQ ID NO: 102; SEQ ID NO: 85 and SEQ ID NO: 86; SEQ ID NO: 85 and SEQ ID NO: 87; SEQ ID NO: 85 and SEQ ID NO: 88; SEQ ID NO: 85 and SEQ ID NO: 89; SEQ ID NO: 85 and SEQ ID NO: 90; SEQ ID NO: 85 and SEQ ID NO: 91; SEQ ID NO: 85 and SEQ ID NO: 92; SEQ ID NO: 85 and SEQ ID NO: 93; SEQ ID NO: 85 and SEQ ID NO: 94; SEQ ID NO: 85 and SEQ ID NO: 95; SEQ ID NO: 85 and SEQ ID NO: 96; SEQ ID NO: 85 and SEQ ID NO: 97; SEQ ID NO: 85 and SEQ ID NO: 98; SEQ ID NO: 85 and SEQ ID NO: 99; SEQ ID NO: 85 and SEQ ID NO: 100; SEQ ID NO: 85 and SEQ ID NO: 101; SEQ ID NO: 85 and SEQ ID NO: 102; SEQ ID NO: 86 and SEQ ID NO: 87; SEQ ID NO: 86 and SEQ ID NO: 88; SEQ ID NO: 86 and SEQ ID NO: 89; SEQ ID NO: 86 and SEQ ID NO: 90; SEQ ID NO: 86 and SEQ ID NO: 91; SEQ ID NO: 86 and SEQ ID NO: 92; SEQ ID NO: 86 and SEQ ID NO: 93; SEQ ID NO: 86 and SEQ ID NO: 94; SEQ ID NO: 86 and SEQ ID NO: 95; SEQ ID NO: 86 and SEQ ID NO: 96; SEQ ID NO: 86 and SEQ ID NO: 97; SEQ ID NO: 86 and SEQ ID NO: 98; SEQ ID NO: 86 and SEQ ID NO: 99; SEQ ID NO: 86 and SEQ ID NO: 100; SEQ ID NO: 86 and SEQ ID NO: 101; SEQ ID NO: 86 and SEQ ID NO: 102; SEQ ID NO: 87 and SEQ ID NO: 88; SEQ ID NO: 87 and SEQ ID NO: 89; SEQ ID NO: 87 and SEQ ID NO: 90; SEQ ID NO: 87 and SEQ ID NO: 91; SEQ ID NO: 87 and SEQ ID NO: 92; SEQ ID NO: 87 and SEQ ID NO: 93; SEQ ID NO: 87 and SEQ ID NO: 94; SEQ ID NO: 87 and SEQ ID NO: 95; SEQ ID NO: 87 and SEQ ID NO: 96; SEQ ID NO: 87 and SEQ ID NO: 97; SEQ ID NO: 87 and SEQ ID NO: 98; SEQ ID NO: 87 and SEQ ID NO: 99; SEQ ID NO: 87 and SEQ ID NO: 100; SEQ ID NO: 87 and SEQ ID NO: 101; SEQ ID NO: 87 and SEQ ID NO: 102; SEQ ID NO: 88 and SEQ ID NO: 89; SEQ ID NO: 88 and SEQ ID NO: 90; SEQ ID NO: 88 and SEQ ID NO: 91; SEQ ID NO: 88 and SEQ ID NO: 92; SEQ ID NO: 88 and SEQ ID NO: 93; SEQ ID NO: 88 and SEQ ID NO: 94; SEQ ID NO: 88 and SEQ ID NO: 95; SEQ ID NO: 88 and SEQ ID NO: 96; SEQ ID NO: 88 and SEQ ID NO: 97; SEQ ID NO: 88 and SEQ ID NO: 98; SEQ ID NO: 88 and SEQ ID NO: 99; SEQ ID NO: 88 and SEQ ID NO: 100; SEQ ID NO: 88 and SEQ ID NO: 101; SEQ ID NO: 88 and SEQ ID NO: 102; SEQ ID NO: 89 and SEQ ID NO: 90; SEQ ID NO: 89 and SEQ ID NO: 91; SEQ ID NO: 89 and SEQ ID NO: 92; SEQ ID NO: 89 and SEQ ID NO: 93; SEQ ID NO: 89 and SEQ ID NO: 94; SEQ ID NO: 89 and SEQ ID NO: 95; SEQ ID NO: 89 and SEQ ID NO: 96; SEQ ID NO: 89 and SEQ ID NO: 97; SEQ ID NO: 89 and SEQ ID NO: 98; SEQ ID NO: 89 and SEQ ID NO: 99; SEQ ID NO: 89 and SEQ ID NO: 100; SEQ ID NO: 89 and SEQ ID NO: 101; SEQ ID NO: 89 and SEQ ID NO: 102; SEQ ID NO: 90 and SEQ ID NO: 91; SEQ ID NO: 90 and SEQ ID NO: 92; SEQ ID NO: 90 and SEQ ID NO: 93; SEQ ID NO: 90 and SEQ ID NO: 94; SEQ ID NO: 90 and SEQ ID NO: 95; SEQ ID NO: 90 and SEQ ID NO: 96; SEQ ID NO: 90 and SEQ ID NO: 97; SEQ ID NO: 90 and SEQ ID NO: 98; SEQ ID NO: 90 and SEQ ID NO: 99; SEQ ID NO: 90 and SEQ ID NO: 100; SEQ ID NO: 90 and SEQ ID NO: 101; SEQ ID NO: 90 and SEQ ID NO: 102; SEQ ID NO: 91 and SEQ ID NO: 92; SEQ ID NO: 91 and SEQ ID NO: 93; SEQ ID NO: 91 and SEQ ID NO: 94; SEQ ID NO: 91 and SEQ ID NO: 95; SEQ ID NO: 91 and SEQ ID NO: 96; SEQ ID NO: 91 and SEQ ID NO: 97; SEQ ID NO: 91 and SEQ ID NO: 98; SEQ ID NO: 91 and SEQ ID NO: 99; SEQ ID NO: 91 and SEQ ID NO: 100; SEQ ID NO: 91 and SEQ ID NO: 101; SEQ ID NO: 91 and SEQ ID NO: 102; SEQ ID NO: 92 and SEQ ID NO: 93; SEQ ID NO: 92 and SEQ ID NO: 94; SEQ ID NO: 92 and SEQ ID NO: 95; SEQ ID NO: 92 and SEQ ID NO: 96; SEQ ID NO: 92 and SEQ ID NO: 97; SEQ ID NO: 92 and SEQ ID NO: 98; SEQ ID NO: 92 and SEQ ID NO: 99; SEQ ID NO: 92 and SEQ ID NO: 100; SEQ ID NO: 92 and SEQ ID NO: 101; SEQ ID NO: 92 and SEQ ID NO: 102; SEQ ID NO: 93 and SEQ ID NO: 94; SEQ ID NO: 93 and SEQ ID NO: 95; SEQ ID NO: 93 and SEQ ID NO: 96; SEQ ID NO: 93 and SEQ ID NO: 97; SEQ ID NO: 93 and SEQ ID NO: 98; SEQ ID NO: 93 and SEQ ID NO: 99; SEQ ID NO: 93 and SEQ ID NO: 100; SEQ ID NO: 93 and SEQ ID NO: 101; SEQ ID NO: 93 and SEQ ID NO: 102; SEQ ID NO: 94 and SEQ ID NO: 95; SEQ ID NO: 94 and SEQ ID NO: 96; SEQ ID NO: 94 and SEQ ID NO: 97; SEQ ID NO: 94 and SEQ ID NO: 98; SEQ ID NO: 94 and SEQ ID NO: 99; SEQ ID NO: 94 and SEQ ID NO: 100; SEQ ID NO: 94 and SEQ ID NO: 101; SEQ ID NO: 94 and SEQ ID NO: 102; SEQ ID NO: 95 and SEQ ID NO: 96; SEQ ID NO: 95 and SEQ ID NO: 97; SEQ ID NO: 95 and SEQ ID NO: 98; SEQ ID NO: 95 and SEQ ID NO: 99; SEQ ID NO: 95 and SEQ ID NO: 100; SEQ ID NO: 95 and SEQ ID NO: 101; SEQ ID NO: 95 and SEQ ID NO: 102; SEQ ID NO: 96 and SEQ ID NO: 97; SEQ ID NO: 96 and SEQ ID NO: 98; SEQ ID NO: 96 and SEQ ID NO: 99; SEQ ID NO: 96 and SEQ ID NO: 100; SEQ ID NO: 96 and SEQ ID NO: 101; SEQ ID NO: 96 and SEQ ID NO: 102; SEQ ID NO: 97 and SEQ ID NO: 98; SEQ ID NO: 97 and SEQ ID NO: 99; SEQ ID NO: 97 and SEQ ID NO: 100; SEQ ID NO: 97 and SEQ ID NO: 101; SEQ ID NO: 97 and SEQ ID NO: 102; SEQ ID NO: 98 and SEQ ID NO: 99; SEQ ID NO: 98 and SEQ ID NO: 100; SEQ ID NO: 98 and SEQ ID NO: 101; SEQ ID NO: 98 and SEQ ID NO: 102; SEQ ID NO: 99 and SEQ ID NO: 100; SEQ ID NO: 99 and SEQ ID NO: 101; SEQ ID NO: 99 and SEQ ID NO: 102; SEQ ID NO: 100 and SEQ ID NO: 101; SEQ ID NO: 100 and SEQ ID NO: 102; SEQ ID NO: 101 and SEQ ID NO: 102; and a nucleic acid encoding a Staphylococcus aureus Cas9 (SaCas9), wherein the nucleic acid encoding the SaCas9 may be on the same or different nucleic acid molecule as the pair of guide RNAs.

In some embodiments, methods are provided for treating Duchenne Muscular Dystrophy (DMD), the method comprising delivering to a cell at least one nucleic acid molecule comprising a pair of guide RNAs comprising a first and second spacer sequence, wherein the pair of first and spacer sequences are selected from any one of the following pairs: SEQ ID NO: 200 and SEQ ID NO: 201; SEQ ID NO: 200 and SEQ ID NO: 202; SEQ ID NO: 200 and SEQ ID NO: 203; SEQ ID NO: 200 and SEQ ID NO: 204; SEQ ID NO: 200 and SEQ ID NO: 205; SEQ ID NO: 200 and SEQ ID NO: 206; SEQ ID NO: 200 and SEQ ID NO: 207; SEQ ID NO: 200 and SEQ ID NO: 208; SEQ ID NO: 201 and SEQ ID NO: 202; SEQ ID NO: 201 and SEQ ID NO: 203; SEQ ID NO: 201 and SEQ ID NO: 204; SEQ ID NO: 201 and SEQ ID NO: 205; SEQ ID NO: 201 and SEQ ID NO: 206; SEQ ID NO: 201 and SEQ ID NO: 207; SEQ ID NO: 201 and SEQ ID NO: 208; SEQ ID NO: 202 and SEQ ID NO: 203; SEQ ID NO: 202 and SEQ ID NO: 204; SEQ ID NO: 202 and SEQ ID NO: 205; SEQ ID NO: 202 and SEQ ID NO: 206; SEQ ID NO: 202 and SEQ ID NO: 207; SEQ ID NO: 202 and SEQ ID NO: 208; SEQ ID NO: 203 and SEQ ID NO: 204; SEQ ID NO: 203 and SEQ ID NO: 205; SEQ ID NO: 203 and SEQ ID NO: 206; SEQ ID NO: 203 and SEQ ID NO: 207; SEQ ID NO: 203 and SEQ ID NO: 208; SEQ ID NO: 204 and SEQ ID NO: 205; SEQ ID NO: 204 and SEQ ID NO: 206; SEQ ID NO: 204 and SEQ ID NO: 207; SEQ ID NO: 204 and SEQ ID NO: 208; SEQ ID NO: 205 and SEQ ID NO: 206; SEQ ID NO: 205 and SEQ ID NO: 207; SEQ ID NO: 205 and SEQ ID NO: 208; SEQ ID NO: 206 and SEQ ID NO: 207; SEQ ID NO: 206 and SEQ ID NO: 208; SEQ ID NO: 207 and SEQ ID NO: 208; SEQ ID NO: 209 and SEQ ID NO: 210; SEQ ID NO: 209 and SEQ ID NO: 211; SEQ ID NO: 209 and SEQ ID NO: 212; SEQ ID NO: 209 and SEQ ID NO: 213; SEQ ID NO: 209 and SEQ ID NO: 214; SEQ ID NO: 209 and SEQ ID NO: 215; SEQ ID NO: 209 and SEQ ID NO: 216; SEQ ID NO: 209 and SEQ ID NO: 217; SEQ ID NO: 209 and SEQ ID NO: 218; SEQ ID NO: 209 and SEQ ID NO: 219; SEQ ID NO: 209 and SEQ ID NO: 220; SEQ ID NO: 209 and SEQ ID NO: 221; SEQ ID NO: 209 and SEQ ID NO: 222; SEQ ID NO: 209 and SEQ ID NO: 223; SEQ ID NO: 209 and SEQ ID NO: 224; SEQ ID NO: 209 and SEQ ID NO: 225; SEQ ID NO: 209 and SEQ ID NO: 226; SEQ ID NO: 209 and SEQ ID NO: 227; SEQ ID NO: 209 and SEQ ID NO: 228; SEQ ID NO: 209 and SEQ ID NO: 229; SEQ ID NO: 209 and SEQ ID NO: 230; SEQ ID NO: 210 and SEQ ID NO: 211; SEQ ID NO: 210 and SEQ ID NO: 212; SEQ ID NO: 210 and SEQ ID NO: 213; SEQ ID NO: 210 and SEQ ID NO: 214; SEQ ID NO: 210 and SEQ ID NO: 215; SEQ ID NO: 210 and SEQ ID NO: 216; SEQ ID NO: 210 and SEQ ID NO: 217; SEQ ID NO: 210 and SEQ ID NO: 218; SEQ ID NO: 210 and SEQ ID NO: 219; SEQ ID NO: 210 and SEQ ID NO: 220; SEQ ID NO: 210 and SEQ ID NO: 221; SEQ ID NO: 210 and SEQ ID NO: 222; SEQ ID NO: 210 and SEQ ID NO: 223; SEQ ID NO: 210 and SEQ ID NO: 224; SEQ ID NO: 210 and SEQ ID NO: 225; SEQ ID NO: 210 and SEQ ID NO: 226; SEQ ID NO: 210 and SEQ ID NO: 227; SEQ ID NO: 210 and SEQ ID NO: 228; SEQ ID NO: 210 and SEQ ID NO: 229; SEQ ID NO: 210 and SEQ ID NO: 230; SEQ ID NO: 211 and SEQ ID NO: 212; SEQ ID NO: 211 and SEQ ID NO: 213; SEQ ID NO: 211 and SEQ ID NO: 214; SEQ ID NO: 211 and SEQ ID NO: 215; SEQ ID NO: 211 and SEQ ID NO: 216; SEQ ID NO: 211 and SEQ ID NO: 217; SEQ ID NO: 211 and SEQ ID NO: 218; SEQ ID NO: 211 and SEQ ID NO: 219; SEQ ID NO: 211 and SEQ ID NO: 220; SEQ ID NO: 211 and SEQ ID NO: 221; SEQ ID NO: 211 and SEQ ID NO: 222; SEQ ID NO: 211 and SEQ ID NO: 223; SEQ ID NO: 211 and SEQ ID NO: 224; SEQ ID NO: 211 and SEQ ID NO: 225; SEQ ID NO: 211 and SEQ ID NO: 226; SEQ ID NO: 211 and SEQ ID NO: 227; SEQ ID NO: 211 and SEQ ID NO: 228; SEQ ID NO: 211 and SEQ ID NO: 229; SEQ ID NO: 211 and SEQ ID NO: 230; SEQ ID NO: 212 and SEQ ID NO: 213; SEQ ID NO: 212 and SEQ ID NO: 214; SEQ ID NO: 212 and SEQ ID NO: 215; SEQ ID NO: 212 and SEQ ID NO: 216; SEQ ID NO: 212 and SEQ ID NO: 217; SEQ ID NO: 212 and SEQ ID NO: 218; SEQ ID NO: 212 and SEQ ID NO: 219; SEQ ID NO: 212 and SEQ ID NO: 220; SEQ ID NO: 212 and SEQ ID NO: 221; SEQ ID NO: 212 and SEQ ID NO: 222; SEQ ID NO: 212 and SEQ ID NO: 223; SEQ ID NO: 212 and SEQ ID NO: 224; SEQ ID NO: 212 and SEQ ID NO: 225; SEQ ID NO: 212 and SEQ ID NO: 226; SEQ ID NO: 212 and SEQ ID NO: 227; SEQ ID NO: 212 and SEQ ID NO: 228; SEQ ID NO: 212 and SEQ ID NO: 229; SEQ ID NO: 212 and SEQ ID NO: 230; SEQ ID NO: 213 and SEQ ID NO: 214; SEQ ID NO: 213 and SEQ ID NO: 215; SEQ ID NO: 213 and SEQ ID NO: 216; SEQ ID NO: 213 and SEQ ID NO: 217; SEQ ID NO: 213 and SEQ ID NO: 218; SEQ ID NO: 213 and SEQ ID NO: 219; SEQ ID NO: 213 and SEQ ID NO: 220; SEQ ID NO: 213 and SEQ ID NO: 221; SEQ ID NO: 213 and SEQ ID NO: 222; SEQ ID NO: 213 and SEQ ID NO: 223; SEQ ID NO: 213 and SEQ ID NO: 224; SEQ ID NO: 213 and SEQ ID NO: 225; SEQ ID NO: 213 and SEQ ID NO: 226; SEQ ID NO: 213 and SEQ ID NO: 227; SEQ ID NO: 213 and SEQ ID NO: 228; SEQ ID NO: 213 and SEQ ID NO: 229; SEQ ID NO: 213 and SEQ ID NO: 230; SEQ ID NO: 214 and SEQ ID NO: 215; SEQ ID NO: 214 and SEQ ID NO: 216; SEQ ID NO: 214 and SEQ ID NO: 217; SEQ ID NO: 214 and SEQ ID NO: 218; SEQ ID NO: 214 and SEQ ID NO: 219; SEQ ID NO: 214 and SEQ ID NO: 220; SEQ ID NO: 214 and SEQ ID NO: 221; SEQ ID NO: 214 and SEQ ID NO: 222; SEQ ID NO: 214 and SEQ ID NO: 223; SEQ ID NO: 214 and SEQ ID NO: 224; SEQ ID NO: 214 and SEQ ID NO: 225; SEQ ID NO: 214 and SEQ ID NO: 226; SEQ ID NO: 214 and SEQ ID NO: 227; SEQ ID NO: 214 and SEQ ID NO: 228; SEQ ID NO: 214 and SEQ ID NO: 229; SEQ ID NO: 214 and SEQ ID NO: 230; SEQ ID NO: 215 and SEQ ID NO: 216; SEQ ID NO: 215 and SEQ ID NO: 217; SEQ ID NO: 215 and SEQ ID NO: 218; SEQ ID NO: 215 and SEQ ID NO: 219; SEQ ID NO: 215 and SEQ ID NO: 220; SEQ ID NO: 215 and SEQ ID NO: 221; SEQ ID NO: 215 and SEQ ID NO: 222; SEQ ID NO: 215 and SEQ ID NO: 223; SEQ ID NO: 215 and SEQ ID NO: 224; SEQ ID NO: 215 and SEQ ID NO: 225; SEQ ID NO: 215 and SEQ ID NO: 226; SEQ ID NO: 215 and SEQ ID NO: 227; SEQ ID NO: 215 and SEQ ID NO: 228; SEQ ID NO: 215 and SEQ ID NO: 229; SEQ ID NO: 215 and SEQ ID NO: 230; SEQ ID NO: 216 and SEQ ID NO: 217; SEQ ID NO: 216 and SEQ ID NO: 218; SEQ ID NO: 216 and SEQ ID NO: 219; SEQ ID NO: 216 and SEQ ID NO: 220; SEQ ID NO: 216 and SEQ ID NO: 221; SEQ ID NO: 216 and SEQ ID NO: 222; SEQ ID NO: 216 and SEQ ID NO: 223; SEQ ID NO: 216 and SEQ ID NO: 224; SEQ ID NO: 216 and SEQ ID NO: 225; SEQ ID NO: 216 and SEQ ID NO: 226; SEQ ID NO: 216 and SEQ ID NO: 227; SEQ ID NO: 216 and SEQ ID NO: 228; SEQ ID NO: 216 and SEQ ID NO: 229; SEQ ID NO: 216 and SEQ ID NO: 230; SEQ ID NO: 217 and SEQ ID NO: 218; SEQ ID NO: 217 and SEQ ID NO: 219; SEQ ID NO: 217 and SEQ ID NO: 220; SEQ ID NO: 217 and SEQ ID NO: 221; SEQ ID NO: 217 and SEQ ID NO: 222; SEQ ID NO: 217 and SEQ ID NO: 223; SEQ ID NO: 217 and SEQ ID NO: 224; SEQ ID NO: 217 and SEQ ID NO: 225; SEQ ID NO: 217 and SEQ ID NO: 226; SEQ ID NO: 217 and SEQ ID NO: 227; SEQ ID NO: 217 and SEQ ID NO: 228; SEQ ID NO: 217 and SEQ ID NO: 229; SEQ ID NO: 217 and SEQ ID NO: 230; SEQ ID NO: 218 and SEQ ID NO: 219; SEQ ID NO: 218 and SEQ ID NO: 220; SEQ ID NO: 218 and SEQ ID NO: 221; SEQ ID NO: 218 and SEQ ID NO: 222; SEQ ID NO: 218 and SEQ ID NO: 223; SEQ ID NO: 218 and SEQ ID NO: 224; SEQ ID NO: 218 and SEQ ID NO: 225; SEQ ID NO: 218 and SEQ ID NO: 226; SEQ ID NO: 218 and SEQ ID NO: 227; SEQ ID NO: 218 and SEQ ID NO: 228; SEQ ID NO: 218 and SEQ ID NO: 229; SEQ ID NO: 218 and SEQ ID NO: 230; SEQ ID NO: 219 and SEQ ID NO: 220; SEQ ID NO: 219 and SEQ ID NO: 221; SEQ ID NO: 219 and SEQ ID NO: 222; SEQ ID NO: 219 and SEQ ID NO: 223; SEQ ID NO: 219 and SEQ ID NO: 224; SEQ ID NO: 219 and SEQ ID NO: 225; SEQ ID NO: 219 and SEQ ID NO: 226; SEQ ID NO: 219 and SEQ ID NO: 227; SEQ ID NO: 219 and SEQ ID NO: 228; SEQ ID NO: 219 and SEQ ID NO: 229; SEQ ID NO: 219 and SEQ ID NO: 230; SEQ ID NO: 220 and SEQ ID NO: 221; SEQ ID NO: 220 and SEQ ID NO: 222; SEQ ID NO: 220 and SEQ ID NO: 223; SEQ ID NO: 220 and SEQ ID NO: 224; SEQ ID NO: 220 and SEQ ID NO: 225; SEQ ID NO: 220 and SEQ ID NO: 226; SEQ ID NO: 220 and SEQ ID NO: 227; SEQ ID NO: 220 and SEQ ID NO: 228; SEQ ID NO: 220 and SEQ ID NO: 229; SEQ ID NO: 220 and SEQ ID NO: 230; SEQ ID NO: 221 and SEQ ID NO: 222; SEQ ID NO: 221 and SEQ ID NO: 223; SEQ ID NO: 221 and SEQ ID NO: 224; SEQ ID NO: 221 and SEQ ID NO: 225; SEQ ID NO: 221 and SEQ ID NO: 226; SEQ ID NO: 221 and SEQ ID NO: 227; SEQ ID NO: 221 and SEQ ID NO: 228; SEQ ID NO: 221 and SEQ ID NO: 229; SEQ ID NO: 221 and SEQ ID NO: 230; SEQ ID NO: 222 and SEQ ID NO: 223; SEQ ID NO: 222 and SEQ ID NO: 224; SEQ ID NO: 222 and SEQ ID NO: 225; SEQ ID NO: 222 and SEQ ID NO: 226; SEQ ID NO: 222 and SEQ ID NO: 227; SEQ ID NO: 222 and SEQ ID NO: 228; SEQ ID NO: 222 and SEQ ID NO: 229; SEQ ID NO: 222 and SEQ ID NO: 230; SEQ ID NO: 223 and SEQ ID NO: 224; SEQ ID NO: 223 and SEQ ID NO: 225; SEQ ID NO: 223 and SEQ ID NO: 226; SEQ ID NO: 223 and SEQ ID NO: 227; SEQ ID NO: 223 and SEQ ID NO: 228; SEQ ID NO: 223 and SEQ ID NO: 229; SEQ ID NO: 223 and SEQ ID NO: 230; SEQ ID NO: 224 and SEQ ID NO: 225; SEQ ID NO: 224 and SEQ ID NO: 226; SEQ ID NO: 224 and SEQ ID NO: 227; SEQ ID NO: 224 and SEQ ID NO: 228; SEQ ID NO: 224 and SEQ ID NO: 229; SEQ ID NO: 224 and SEQ ID NO: 230; SEQ ID NO: 225 and SEQ ID NO: 226; SEQ ID NO: 225 and SEQ ID NO: 227; SEQ ID NO: 225 and SEQ ID NO: 228; SEQ ID NO: 225 and SEQ ID NO: 229; SEQ ID NO: 225 and SEQ ID NO: 230; SEQ ID NO: 226 and SEQ ID NO: 227; SEQ ID NO: 226 and SEQ ID NO: 228; SEQ ID NO: 226 and SEQ ID NO: 229; SEQ ID NO: 226 and SEQ ID NO: 230; SEQ ID NO: 227 and SEQ ID NO: 228; SEQ ID NO: 227 and SEQ ID NO: 229; SEQ ID NO: 227 and SEQ ID NO: 230; SEQ ID NO: 228 and SEQ ID NO: 229; SEQ ID NO: 228 and SEQ ID NO: 230; SEQ ID NO: 229 and SEQ ID NO: 230; SEQ ID NO: 231 and SEQ ID NO: 232; SEQ ID NO: 231 and SEQ ID NO: 233; SEQ ID NO: 231 and SEQ ID NO: 234; SEQ ID NO: 231 and SEQ ID NO: 235; SEQ ID NO: 231 and SEQ ID NO: 236; SEQ ID NO: 231 and SEQ ID NO: 237; SEQ ID NO: 231 and SEQ ID NO: 238; SEQ ID NO: 231 and SEQ ID NO: 239; SEQ ID NO: 231 and SEQ ID NO: 240; SEQ ID NO: 231 and SEQ ID NO: 241; SEQ ID NO: 231 and SEQ ID NO: 242; SEQ ID NO: 232 and SEQ ID NO: 233; SEQ ID NO: 232 and SEQ ID NO: 234; SEQ ID NO: 232 and SEQ ID NO: 235; SEQ ID NO: 232 and SEQ ID NO: 236; SEQ ID NO: 232 and SEQ ID NO: 237; SEQ ID NO: 232 and SEQ ID NO: 238; SEQ ID NO: 232 and SEQ ID NO: 239; SEQ ID NO: 232 and SEQ ID NO: 240; SEQ ID NO: 232 and SEQ ID NO: 241; SEQ ID NO: 232 and SEQ ID NO: 242; SEQ ID NO: 233 and SEQ ID NO: 234; SEQ ID NO: 233 and SEQ ID NO: 235; SEQ ID NO: 233 and SEQ ID NO: 236; SEQ ID NO: 233 and SEQ ID NO: 237; SEQ ID NO: 233 and SEQ ID NO: 238; SEQ ID NO: 233 and SEQ ID NO: 239; SEQ ID NO: 233 and SEQ ID NO: 240; SEQ ID NO: 233 and SEQ ID NO: 241; SEQ ID NO: 233 and SEQ ID NO: 242; SEQ ID NO: 234 and SEQ ID NO: 235; SEQ ID NO: 234 and SEQ ID NO: 236; SEQ ID NO: 234 and SEQ ID NO: 237; SEQ ID NO: 234 and SEQ ID NO: 238; SEQ ID NO: 234 and SEQ ID NO: 239; SEQ ID NO: 234 and SEQ ID NO: 240; SEQ ID NO: 234 and SEQ ID NO: 241; SEQ ID NO: 234 and SEQ ID NO: 242; SEQ ID NO: 235 and SEQ ID NO: 236; SEQ ID NO: 235 and SEQ ID NO: 237; SEQ ID NO: 235 and SEQ ID NO: 238; SEQ ID NO: 235 and SEQ ID NO: 239; SEQ ID NO: 235 and SEQ ID NO: 240; SEQ ID NO: 235 and SEQ ID NO: 241; SEQ ID NO: 235 and SEQ ID NO: 242; SEQ ID NO: 236 and SEQ ID NO: 237; SEQ ID NO: 236 and SEQ ID NO: 238; SEQ ID NO: 236 and SEQ ID NO: 239; SEQ ID NO: 236 and SEQ ID NO: 240; SEQ ID NO: 236 and SEQ ID NO: 241; SEQ ID NO: 236 and SEQ ID NO: 242; SEQ ID NO: 237 and SEQ ID NO: 238; SEQ ID NO: 237 and SEQ ID NO: 239; SEQ ID NO: 237 and SEQ ID NO: 240; SEQ ID NO: 237 and SEQ ID NO: 241; SEQ ID NO: 237 and SEQ ID NO: 242; SEQ ID NO: 238 and SEQ ID NO: 239; SEQ ID NO: 238 and SEQ ID NO: 240; SEQ ID NO: 238 and SEQ ID NO: 241; SEQ ID NO: 238 and SEQ ID NO: 242; SEQ ID NO: 239 and SEQ ID NO: 240; SEQ ID NO: 239 and SEQ ID NO: 241; SEQ ID NO: 239 and SEQ ID NO: 242; SEQ ID NO: 240 and SEQ ID NO: 241; SEQ ID NO: 240 and SEQ ID NO: 242; SEQ ID NO: 241 and SEQ ID NO: 242; SEQ ID NO: 243 and SEQ ID NO: 244; SEQ ID NO: 243 and SEQ ID NO: 245; SEQ ID NO: 243 and SEQ ID NO: 246; SEQ ID NO: 243 and SEQ ID NO: 247; SEQ ID NO: 243 and SEQ ID NO: 248; SEQ ID NO: 243 and SEQ ID NO: 249; SEQ ID NO: 243 and SEQ ID NO: 250; SEQ ID NO: 243 and SEQ ID NO: 251; SEQ ID NO: 243 and SEQ ID NO: 252; SEQ ID NO: 243 and SEQ ID NO: 253; SEQ ID NO: 243 and SEQ ID NO: 254; SEQ ID NO: 243 and SEQ ID NO: 255; SEQ ID NO: 243 and SEQ ID NO: 256; SEQ ID NO: 243 and SEQ ID NO: 257; SEQ ID NO: 243 and SEQ ID NO: 258; SEQ ID NO: 243 and SEQ ID NO: 259; SEQ ID NO: 243 and SEQ ID NO: 260; SEQ ID NO: 243 and SEQ ID NO: 261; SEQ ID NO: 243 and SEQ ID NO: 262; SEQ ID NO: 243 and SEQ ID NO: 263; SEQ ID NO: 243 and SEQ ID NO: 264; SEQ ID NO: 243 and SEQ ID NO: 265; SEQ ID NO: 243 and SEQ ID NO: 266; SEQ ID NO: 243 and SEQ ID NO: 267; SEQ ID NO: 243 and SEQ ID NO: 268; SEQ ID NO: 243 and SEQ ID NO: 269; SEQ ID NO: 244 and SEQ ID NO: 245; SEQ ID NO: 244 and SEQ ID NO: 246; SEQ ID NO: 244 and SEQ ID NO: 247; SEQ ID NO: 244 and SEQ ID NO: 248; SEQ ID NO: 244 and SEQ ID NO: 249; SEQ ID NO: 244 and SEQ ID NO: 250; SEQ ID NO: 244 and SEQ ID NO: 251; SEQ ID NO: 244 and SEQ ID NO: 252; SEQ ID NO: 244 and SEQ ID NO: 253; SEQ ID NO: 244 and SEQ ID NO: 254; SEQ ID NO: 244 and SEQ ID NO: 255; SEQ ID NO: 244 and SEQ ID NO: 256; SEQ ID NO: 244 and SEQ ID NO: 257; SEQ ID NO: 244 and SEQ ID NO: 258; SEQ ID NO: 244 and SEQ ID NO: 259; SEQ ID NO: 244 and SEQ ID NO: 260; SEQ ID NO: 244 and SEQ ID NO: 261; SEQ ID NO: 244 and SEQ ID NO: 262; SEQ ID NO: 244 and SEQ ID NO: 263; SEQ ID NO: 244 and SEQ ID NO: 264; SEQ ID NO: 244 and SEQ ID NO: 265; SEQ ID NO: 244 and SEQ ID NO: 266; SEQ ID NO: 244 and SEQ ID NO: 267; SEQ ID NO: 244 and SEQ ID NO: 268; SEQ ID NO: 244 and SEQ ID NO: 269; SEQ ID NO: 245 and SEQ ID NO: 246; SEQ ID NO: 245 and SEQ ID NO: 247; SEQ ID NO: 245 and SEQ ID NO: 248; SEQ ID NO: 245 and SEQ ID NO: 249; SEQ ID NO: 245 and SEQ ID NO: 250; SEQ ID NO: 245 and SEQ ID NO: 251; SEQ ID NO: 245 and SEQ ID NO: 252; SEQ ID NO: 245 and SEQ ID NO: 253; SEQ ID NO: 245 and SEQ ID NO: 254; SEQ ID NO: 245 and SEQ ID NO: 255; SEQ ID NO: 245 and SEQ ID NO: 256; SEQ ID NO: 245 and SEQ ID NO: 257; SEQ ID NO: 245 and SEQ ID NO: 258; SEQ ID NO: 245 and SEQ ID NO: 259; SEQ ID NO: 245 and SEQ ID NO: 260; SEQ ID NO: 245 and SEQ ID NO: 261; SEQ ID NO: 245 and SEQ ID NO: 262; SEQ ID NO: 245 and SEQ ID NO: 263; SEQ ID NO: 245 and SEQ ID NO: 264; SEQ ID NO: 245 and SEQ ID NO: 265; SEQ ID NO: 245 and SEQ ID NO: 266; SEQ ID NO: 245 and SEQ ID NO: 267; SEQ ID NO: 245 and SEQ ID NO: 268; SEQ ID NO: 245 and SEQ ID NO: 269; SEQ ID NO: 246 and SEQ ID NO: 247; SEQ ID NO: 246 and SEQ ID NO: 248; SEQ ID NO: 246 and SEQ ID NO: 249; SEQ ID NO: 246 and SEQ ID NO: 250; SEQ ID NO: 246 and SEQ ID NO: 251; SEQ ID NO: 246 and SEQ ID NO: 252; SEQ ID NO: 246 and SEQ ID NO: 253; SEQ ID NO: 246 and SEQ ID NO: 254; SEQ ID NO: 246 and SEQ ID NO: 255; SEQ ID NO: 246 and SEQ ID NO: 256; SEQ ID NO: 246 and SEQ ID NO: 257; SEQ ID NO: 246 and SEQ ID NO: 258; SEQ ID NO: 246 and SEQ ID NO: 259; SEQ ID NO: 246 and SEQ ID NO: 260; SEQ ID NO: 246 and SEQ ID NO: 261; SEQ ID NO: 246 and SEQ ID NO: 262; SEQ ID NO: 246 and SEQ ID NO: 263; SEQ ID NO: 246 and SEQ ID NO: 264; SEQ ID NO: 246 and SEQ ID NO: 265; SEQ ID NO: 246 and SEQ ID NO: 266; SEQ ID NO: 246 and SEQ ID NO: 267; SEQ ID NO: 246 and SEQ ID NO: 268; SEQ ID NO: 246 and SEQ ID NO: 269; SEQ ID NO: 247 and SEQ ID NO: 248; SEQ ID NO: 247 and SEQ ID NO: 249; SEQ ID NO: 247 and SEQ ID NO: 250; SEQ ID NO: 247 and SEQ ID NO: 251; SEQ ID NO: 247 and SEQ ID NO: 252; SEQ ID NO: 247 and SEQ ID NO: 253; SEQ ID NO: 247 and SEQ ID NO: 254; SEQ ID NO: 247 and SEQ ID NO: 255; SEQ ID NO: 247 and SEQ ID NO: 256; SEQ ID NO: 247 and SEQ ID NO: 257; SEQ ID NO: 247 and SEQ ID NO: 258; SEQ ID NO: 247 and SEQ ID NO: 259; SEQ ID NO: 247 and SEQ ID NO: 260; SEQ ID NO: 247 and SEQ ID NO: 261; SEQ ID NO: 247 and SEQ ID NO: 262; SEQ ID NO: 247 and SEQ ID NO: 263; SEQ ID NO: 247 and SEQ ID NO: 264; SEQ ID NO: 247 and SEQ ID NO: 265; SEQ ID NO: 247 and SEQ ID NO: 266; SEQ ID NO: 247 and SEQ ID NO: 267; SEQ ID NO: 247 and SEQ ID NO: 268; SEQ ID NO: 247 and SEQ ID NO: 269; SEQ ID NO: 248 and SEQ ID NO: 249; SEQ ID NO: 248 and SEQ ID NO: 250; SEQ ID NO: 248 and SEQ ID NO: 251; SEQ ID NO: 248 and SEQ ID NO: 252; SEQ ID NO: 248 and SEQ ID NO: 253; SEQ ID NO: 248 and SEQ ID NO: 254; SEQ ID NO: 248 and SEQ ID NO: 255; SEQ ID NO: 248 and SEQ ID NO: 256; SEQ ID NO: 248 and SEQ ID NO: 257; SEQ ID NO: 248 and SEQ ID NO: 258; SEQ ID NO: 248 and SEQ ID NO: 259; SEQ ID NO: 248 and SEQ ID NO: 260; SEQ ID NO: 248 and SEQ ID NO: 261; SEQ ID NO: 248 and SEQ ID NO: 262; SEQ ID NO: 248 and SEQ ID NO: 263; SEQ ID NO: 248 and SEQ ID NO: 264; SEQ ID NO: 248 and SEQ ID NO: 265; SEQ ID NO: 248 and SEQ ID NO: 266; SEQ ID NO: 248 and SEQ ID NO: 267; SEQ ID NO: 248 and SEQ ID NO: 268; SEQ ID NO: 248 and SEQ ID NO: 269; SEQ ID NO: 249 and SEQ ID NO: 250; SEQ ID NO: 249 and SEQ ID NO: 251; SEQ ID NO: 249 and SEQ ID NO: 252; SEQ ID NO: 249 and SEQ ID NO: 253; SEQ ID NO: 249 and SEQ ID NO: 254; SEQ ID NO: 249 and SEQ ID NO: 255; SEQ ID NO: 249 and SEQ ID NO: 256; SEQ ID NO: 249 and SEQ ID NO: 257; SEQ ID NO: 249 and SEQ ID NO: 258; SEQ ID NO: 249 and SEQ ID NO: 259; SEQ ID NO: 249 and SEQ ID NO: 260; SEQ ID NO: 249 and SEQ ID NO: 261; SEQ ID NO: 249 and SEQ ID NO: 262; SEQ ID NO: 249 and SEQ ID NO: 263; SEQ ID NO: 249 and SEQ ID NO: 264; SEQ ID NO: 249 and SEQ ID NO: 265; SEQ ID NO: 249 and SEQ ID NO: 266; SEQ ID NO: 249 and SEQ ID NO: 267; SEQ ID NO: 249 and SEQ ID NO: 268; SEQ ID NO: 249 and SEQ ID NO: 269; SEQ ID NO: 250 and SEQ ID NO: 251; SEQ ID NO: 250 and SEQ ID NO: 252; SEQ ID NO: 250 and SEQ ID NO: 253; SEQ ID NO: 250 and SEQ ID NO: 254; SEQ ID NO: 250 and SEQ ID NO: 255; SEQ ID NO: 250 and SEQ ID NO: 256; SEQ ID NO: 250 and SEQ ID NO: 257; SEQ ID NO: 250 and SEQ ID NO: 258; SEQ ID NO: 250 and SEQ ID NO: 259; SEQ ID NO: 250 and SEQ ID NO: 260; SEQ ID NO: 250 and SEQ ID NO: 261; SEQ ID NO: 250 and SEQ ID NO: 262; SEQ ID NO: 250 and SEQ ID NO: 263; SEQ ID NO: 250 and SEQ ID NO: 264; SEQ ID NO: 250 and SEQ ID NO: 265; SEQ ID NO: 250 and SEQ ID NO: 266; SEQ ID NO: 250 and SEQ ID NO: 267; SEQ ID NO: 250 and SEQ ID NO: 268; SEQ ID NO: 250 and SEQ ID NO: 269; SEQ ID NO: 251 and SEQ ID NO: 252; SEQ ID NO: 251 and SEQ ID NO: 253; SEQ ID NO: 251 and SEQ ID NO: 254; SEQ ID NO: 251 and SEQ ID NO: 255; SEQ ID NO: 251 and SEQ ID NO: 256; SEQ ID NO: 251 and SEQ ID NO: 257; SEQ ID NO: 251 and SEQ ID NO: 258; SEQ ID NO: 251 and SEQ ID NO: 259; SEQ ID NO: 251 and SEQ ID NO: 260; SEQ ID NO: 251 and SEQ ID NO: 261; SEQ ID NO: 251 and SEQ ID NO: 262; SEQ ID NO: 251 and SEQ ID NO: 263; SEQ ID NO: 251 and SEQ ID NO: 264; SEQ ID NO: 251 and SEQ ID NO: 265; SEQ ID NO: 251 and SEQ ID NO: 266; SEQ ID NO: 251 and SEQ ID NO: 267; SEQ ID NO: 251 and SEQ ID NO: 268; SEQ ID NO: 251 and SEQ ID NO: 269; SEQ ID NO: 252 and SEQ ID NO: 253; SEQ ID NO: 252 and SEQ ID NO: 254; SEQ ID NO: 252 and SEQ ID NO: 255; SEQ ID NO: 252 and SEQ ID NO: 256; SEQ ID NO: 252 and SEQ ID NO: 257; SEQ ID NO: 252 and SEQ ID NO: 258; SEQ ID NO: 252 and SEQ ID NO: 259; SEQ ID NO: 252 and SEQ ID NO: 260; SEQ ID NO: 252 and SEQ ID NO: 261; SEQ ID NO: 252 and SEQ ID NO: 262; SEQ ID NO: 252 and SEQ ID NO: 263; SEQ ID NO: 252 and SEQ ID NO: 264; SEQ ID NO: 252 and SEQ ID NO: 265; SEQ ID NO: 252 and SEQ ID NO: 266; SEQ ID NO: 252 and SEQ ID NO: 267; SEQ ID NO: 252 and SEQ ID NO: 268; SEQ ID NO: 252 and SEQ ID NO: 269; SEQ ID NO: 253 and SEQ ID NO: 254; SEQ ID NO: 253 and SEQ ID NO: 255; SEQ ID NO: 253 and SEQ ID NO: 256; SEQ ID NO: 253 and SEQ ID NO: 257; SEQ ID NO: 253 and SEQ ID NO: 258; SEQ ID NO: 253 and SEQ ID NO: 259; SEQ ID NO: 253 and SEQ ID NO: 260; SEQ ID NO: 253 and SEQ ID NO: 261; SEQ ID NO: 253 and SEQ ID NO: 262; SEQ ID NO: 253 and SEQ ID NO: 263; SEQ ID NO: 253 and SEQ ID NO: 264; SEQ ID NO: 253 and SEQ ID NO: 265; SEQ ID NO: 253 and SEQ ID NO: 266; SEQ ID NO: 253 and SEQ ID NO: 267; SEQ ID NO: 253 and SEQ ID NO: 268; SEQ ID NO: 253 and SEQ ID NO: 269; SEQ ID NO: 254 and SEQ ID NO: 255; SEQ ID NO: 254 and SEQ ID NO: 256; SEQ ID NO: 254 and SEQ ID NO: 257; SEQ ID NO: 254 and SEQ ID NO: 258; SEQ ID NO: 254 and SEQ ID NO: 259; SEQ ID NO: 254 and SEQ ID NO: 260; SEQ ID NO: 254 and SEQ ID NO: 261; SEQ ID NO: 254 and SEQ ID NO: 262; SEQ ID NO: 254 and SEQ ID NO: 263; SEQ ID NO: 254 and SEQ ID NO: 264; SEQ ID NO: 254 and SEQ ID NO: 265; SEQ ID NO: 254 and SEQ ID NO: 266; SEQ ID NO: 254 and SEQ ID NO: 267; SEQ ID NO: 254 and SEQ ID NO: 268; SEQ ID NO: 254 and SEQ ID NO: 269; SEQ ID NO: 255 and SEQ ID NO: 256; SEQ ID NO: 255 and SEQ ID NO: 257; SEQ ID NO: 255 and SEQ ID NO: 258; SEQ ID NO: 255 and SEQ ID NO: 259; SEQ ID NO: 255 and SEQ ID NO: 260; SEQ ID NO: 255 and SEQ ID NO: 261; SEQ ID NO: 255 and SEQ ID NO: 262; SEQ ID NO: 255 and SEQ ID NO: 263; SEQ ID NO: 255 and SEQ ID NO: 264; SEQ ID NO: 255 and SEQ ID NO: 265; SEQ ID NO: 255 and SEQ ID NO: 266; SEQ ID NO: 255 and SEQ ID NO: 267; SEQ ID NO: 255 and SEQ ID NO: 268; SEQ ID NO: 255 and SEQ ID NO: 269; SEQ ID NO: 256 and SEQ ID NO: 257; SEQ ID NO: 256 and SEQ ID NO: 258; SEQ ID NO: 256 and SEQ ID NO: 259; SEQ ID NO: 256 and SEQ ID NO: 260; SEQ ID NO: 256 and SEQ ID NO: 261; SEQ ID NO: 256 and SEQ ID NO: 262; SEQ ID NO: 256 and SEQ ID NO: 263; SEQ ID NO: 256 and SEQ ID NO: 264; SEQ ID NO: 256 and SEQ ID NO: 265; SEQ ID NO: 256 and SEQ ID NO: 266; SEQ ID NO: 256 and SEQ ID NO: 267; SEQ ID NO: 256 and SEQ ID NO: 268; SEQ ID NO: 256 and SEQ ID NO: 269; SEQ ID NO: 257 and SEQ ID NO: 258; SEQ ID NO: 257 and SEQ ID NO: 259; SEQ ID NO: 257 and SEQ ID NO: 260; SEQ ID NO: 257 and SEQ ID NO: 261; SEQ ID NO: 257 and SEQ ID NO: 262; SEQ ID NO: 257 and SEQ ID NO: 263; SEQ ID NO: 257 and SEQ ID NO: 264; SEQ ID NO: 257 and SEQ ID NO: 265; SEQ ID NO: 257 and SEQ ID NO: 266; SEQ ID NO: 257 and SEQ ID NO: 267; SEQ ID NO: 257 and SEQ ID NO: 268; SEQ ID NO: 257 and SEQ ID NO: 269; SEQ ID NO: 258 and SEQ ID NO: 259; SEQ ID NO: 258 and SEQ ID NO: 260; SEQ ID NO: 258 and SEQ ID NO: 261; SEQ ID NO: 258 and SEQ ID NO: 262; SEQ ID NO: 258 and SEQ ID NO: 263; SEQ ID NO: 258 and SEQ ID NO: 264; SEQ ID NO: 258 and SEQ ID NO: 265; SEQ ID NO: 258 and SEQ ID NO: 266; SEQ ID NO: 258 and SEQ ID NO: 267; SEQ ID NO: 258 and SEQ ID NO: 268; SEQ ID NO: 258 and SEQ ID NO: 269; SEQ ID NO: 259 and SEQ ID NO: 260; SEQ ID NO: 259 and SEQ ID NO: 261; SEQ ID NO: 259 and SEQ ID NO: 262; SEQ ID NO: 259 and SEQ ID NO: 263; SEQ ID NO: 259 and SEQ ID NO: 264; SEQ ID NO: 259 and SEQ ID NO: 265; SEQ ID NO: 259 and SEQ ID NO: 266; SEQ ID NO: 259 and SEQ ID NO: 267; SEQ ID NO: 259 and SEQ ID NO: 268; SEQ ID NO: 259 and SEQ ID NO: 269; SEQ ID NO: 260 and SEQ ID NO: 261; SEQ ID NO: 260 and SEQ ID NO: 262; SEQ ID NO: 260 and SEQ ID NO: 263; SEQ ID NO: 260 and SEQ ID NO: 264; SEQ ID NO: 260 and SEQ ID NO: 265; SEQ ID NO: 260 and SEQ ID NO: 266; SEQ ID NO: 260 and SEQ ID NO: 267; SEQ ID NO: 260 and SEQ ID NO: 268; SEQ ID NO: 260 and SEQ ID NO: 269; SEQ ID NO: 261 and SEQ ID NO: 262; SEQ ID NO: 261 and SEQ ID NO: 263; SEQ ID NO: 261 and SEQ ID NO: 264; SEQ ID NO: 261 and SEQ ID NO: 265; SEQ ID NO: 261 and SEQ ID NO: 266; SEQ ID NO: 261 and SEQ ID NO: 267; SEQ ID NO: 261 and SEQ ID NO: 268; SEQ ID NO: 261 and SEQ ID NO: 269; SEQ ID NO: 262 and SEQ ID NO: 263; SEQ ID NO: 262 and SEQ ID NO: 264; SEQ ID NO: 262 and SEQ ID NO: 265; SEQ ID NO: 262 and SEQ ID NO: 266; SEQ ID NO: 262 and SEQ ID NO: 267; SEQ ID NO: 262 and SEQ ID NO: 268; SEQ ID NO: 262 and SEQ ID NO: 269; SEQ ID NO: 263 and SEQ ID NO: 264; SEQ ID NO: 263 and SEQ ID NO: 265; SEQ ID NO: 263 and SEQ ID NO: 266; SEQ ID NO: 263 and SEQ ID NO: 267; SEQ ID NO: 263 and SEQ ID NO: 268; SEQ ID NO: 263 and SEQ ID NO: 269; SEQ ID NO: 264 and SEQ ID NO: 265; SEQ ID NO: 264 and SEQ ID NO: 266; SEQ ID NO: 264 and SEQ ID NO: 267; SEQ ID NO: 264 and SEQ ID NO: 268; SEQ ID NO: 264 and SEQ ID NO: 269; SEQ ID NO: 265 and SEQ ID NO: 266; SEQ ID NO: 265 and SEQ ID NO: 267; SEQ ID NO: 265 and SEQ ID NO: 268; SEQ ID NO: 265 and SEQ ID NO: 269; SEQ ID NO: 266 and SEQ ID NO: 267; SEQ ID NO: 266 and SEQ ID NO: 268; SEQ ID NO: 266 and SEQ ID NO: 269; SEQ ID NO: 267 and SEQ ID NO: 268; SEQ ID NO: 267 and SEQ ID NO: 269; SEQ ID NO: 268 and SEQ ID NO: 269; SEQ ID NO: 270 and SEQ ID NO: 271; SEQ ID NO: 270 and SEQ ID NO: 272; SEQ ID NO: 270 and SEQ ID NO: 273; SEQ ID NO: 270 and SEQ ID NO: 274; SEQ ID NO: 270 and SEQ ID NO: 275; SEQ ID NO: 270 and SEQ ID NO: 276; SEQ ID NO: 270 and SEQ ID NO: 277; SEQ ID NO: 270 and SEQ ID NO: 278; SEQ ID NO: 270 and SEQ ID NO: 279; SEQ ID NO: 270 and SEQ ID NO: 280; SEQ ID NO: 270 and SEQ ID NO: 281; SEQ ID NO: 270 and SEQ ID NO: 282; SEQ ID NO: 270 and SEQ ID NO: 283; SEQ ID NO: 270 and SEQ ID NO: 284; SEQ ID NO: 270 and SEQ ID NO: 285; SEQ ID NO: 270 and SEQ ID NO: 286; SEQ ID NO: 270 and SEQ ID NO: 287; SEQ ID NO: 270 and SEQ ID NO: 288; SEQ ID NO: 270 and SEQ ID NO: 289; SEQ ID NO: 270 and SEQ ID NO: 290; SEQ ID NO: 270 and SEQ ID NO: 291; SEQ ID NO: 270 and SEQ ID NO: 292; SEQ ID NO: 271 and SEQ ID NO: 272; SEQ ID NO: 271 and SEQ ID NO: 273; SEQ ID NO: 271 and SEQ ID NO: 274; SEQ ID NO: 271 and SEQ ID NO: 275; SEQ ID NO: 271 and SEQ ID NO: 276; SEQ ID NO: 271 and SEQ ID NO: 277; SEQ ID NO: 271 and SEQ ID NO: 278; SEQ ID NO: 271 and SEQ ID NO: 279; SEQ ID NO: 271 and SEQ ID NO: 280; SEQ ID NO: 271 and SEQ ID NO: 281; SEQ ID NO: 271 and SEQ ID NO: 282; SEQ ID NO: 271 and SEQ ID NO: 283; SEQ ID NO: 271 and SEQ ID NO: 284; SEQ ID NO: 271 and SEQ ID NO: 285; SEQ ID NO: 271 and SEQ ID NO: 286; SEQ ID NO: 271 and SEQ ID NO: 287; SEQ ID NO: 271 and SEQ ID NO: 288; SEQ ID NO: 271 and SEQ ID NO: 289; SEQ ID NO: 271 and SEQ ID NO: 290; SEQ ID NO: 271 and SEQ ID NO: 291; SEQ ID NO: 271 and SEQ ID NO: 292; SEQ ID NO: 272 and SEQ ID NO: 273; SEQ ID NO: 272 and SEQ ID NO: 274; SEQ ID NO: 272 and SEQ ID NO: 275; SEQ ID NO: 272 and SEQ ID NO: 276; SEQ ID NO: 272 and SEQ ID NO: 277; SEQ ID NO: 272 and SEQ ID NO: 278; SEQ ID NO: 272 and SEQ ID NO: 279; SEQ ID NO: 272 and SEQ ID NO: 280; SEQ ID NO: 272 and SEQ ID NO: 281; SEQ ID NO: 272 and SEQ ID NO: 282; SEQ ID NO: 272 and SEQ ID NO: 283; SEQ ID NO: 272 and SEQ ID NO: 284; SEQ ID NO: 272 and SEQ ID NO: 285; SEQ ID NO: 272 and SEQ ID NO: 286; SEQ ID NO: 272 and SEQ ID NO: 287; SEQ ID NO: 272 and SEQ ID NO: 288; SEQ ID NO: 272 and SEQ ID NO: 289; SEQ ID NO: 272 and SEQ ID NO: 290; SEQ ID NO: 272 and SEQ ID NO: 291; SEQ ID NO: 272 and SEQ ID NO: 292; SEQ ID NO: 273 and SEQ ID NO: 274; SEQ ID NO: 273 and SEQ ID NO: 275; SEQ ID NO: 273 and SEQ ID NO: 276; SEQ ID NO: 273 and SEQ ID NO: 277; SEQ ID NO: 273 and SEQ ID NO: 278; SEQ ID NO: 273 and SEQ ID NO: 279; SEQ ID NO: 273 and SEQ ID NO: 280; SEQ ID NO: 273 and SEQ ID NO: 281; SEQ ID NO: 273 and SEQ ID NO: 282; SEQ ID NO: 273 and SEQ ID NO: 283; SEQ ID NO: 273 and SEQ ID NO: 284; SEQ ID NO: 273 and SEQ ID NO: 285; SEQ ID NO: 273 and SEQ ID NO: 286; SEQ ID NO: 273 and SEQ ID NO: 287; SEQ ID NO: 273 and SEQ ID NO: 288; SEQ ID NO: 273 and SEQ ID NO: 289; SEQ ID NO: 273 and SEQ ID NO: 290; SEQ ID NO: 273 and SEQ ID NO: 291; SEQ ID NO: 273 and SEQ ID NO: 292; SEQ ID NO: 274 and SEQ ID NO: 275; SEQ ID NO: 274 and SEQ ID NO: 276; SEQ ID NO: 274 and SEQ ID NO: 277; SEQ ID NO: 274 and SEQ ID NO: 278; SEQ ID NO: 274 and SEQ ID NO: 279; SEQ ID NO: 274 and SEQ ID NO: 280; SEQ ID NO: 274 and SEQ ID NO: 281; SEQ ID NO: 274 and SEQ ID NO: 282; SEQ ID NO: 274 and SEQ ID NO: 283; SEQ ID NO: 274 and SEQ ID NO: 284; SEQ ID NO: 274 and SEQ ID NO: 285; SEQ ID NO: 274 and SEQ ID NO: 286; SEQ ID NO: 274 and SEQ ID NO: 287; SEQ ID NO: 274 and SEQ ID NO: 288; SEQ ID NO: 274 and SEQ ID NO: 289; SEQ ID NO: 274 and SEQ ID NO: 290; SEQ ID NO: 274 and SEQ ID NO: 291; SEQ ID NO: 274 and SEQ ID NO: 292; SEQ ID NO: 275 and SEQ ID NO: 276; SEQ ID NO: 275 and SEQ ID NO: 277; SEQ ID NO: 275 and SEQ ID NO: 278; SEQ ID NO: 275 and SEQ ID NO: 279; SEQ ID NO: 275 and SEQ ID NO: 280; SEQ ID NO: 275 and SEQ ID NO: 281; SEQ ID NO: 275 and SEQ ID NO: 282; SEQ ID NO: 275 and SEQ ID NO: 283; SEQ ID NO: 275 and SEQ ID NO: 284; SEQ ID NO: 275 and SEQ ID NO: 285; SEQ ID NO: 275 and SEQ ID NO: 286; SEQ ID NO: 275 and SEQ ID NO: 287; SEQ ID NO: 275 and SEQ ID NO: 288; SEQ ID NO: 275 and SEQ ID NO: 289; SEQ ID NO: 275 and SEQ ID NO: 290; SEQ ID NO: 275 and SEQ ID NO: 291; SEQ ID NO: 275 and SEQ ID NO: 292; SEQ ID NO: 276 and SEQ ID NO: 277; SEQ ID NO: 276 and SEQ ID NO: 278; SEQ ID NO: 276 and SEQ ID NO: 279; SEQ ID NO: 276 and SEQ ID NO: 280; SEQ ID NO: 276 and SEQ ID NO: 281; SEQ ID NO: 276 and SEQ ID NO: 282; SEQ ID NO: 276 and SEQ ID NO: 283; SEQ ID NO: 276 and SEQ ID NO: 284; SEQ ID NO: 276 and SEQ ID NO: 285; SEQ ID NO: 276 and SEQ ID NO: 286; SEQ ID NO: 276 and SEQ ID NO: 287; SEQ ID NO: 276 and SEQ ID NO: 288; SEQ ID NO: 276 and SEQ ID NO: 289; SEQ ID NO: 276 and SEQ ID NO: 290; SEQ ID NO: 276 and SEQ ID NO: 291; SEQ ID NO: 276 and SEQ ID NO: 292; SEQ ID NO: 277 and SEQ ID NO: 278; SEQ ID NO: 277 and SEQ ID NO: 279; SEQ ID NO: 277 and SEQ ID NO: 280; SEQ ID NO: 277 and SEQ ID NO: 281; SEQ ID NO: 277 and SEQ ID NO: 282; SEQ ID NO: 277 and SEQ ID NO: 283; SEQ ID NO: 277 and SEQ ID NO: 284; SEQ ID NO: 277 and SEQ ID NO: 285; SEQ ID NO: 277 and SEQ ID NO: 286; SEQ ID NO: 277 and SEQ ID NO: 287; SEQ ID NO: 277 and SEQ ID NO: 288; SEQ ID NO: 277 and SEQ ID NO: 289; SEQ ID NO: 277 and SEQ ID NO: 290; SEQ ID NO: 277 and SEQ ID NO: 291; SEQ ID NO: 277 and SEQ ID NO: 292; SEQ ID NO: 278 and SEQ ID NO: 279; SEQ ID NO: 278 and SEQ ID NO: 280; SEQ ID NO: 278 and SEQ ID NO: 281; SEQ ID NO: 278 and SEQ ID NO: 282; SEQ ID NO: 278 and SEQ ID NO: 283; SEQ ID NO: 278 and SEQ ID NO: 284; SEQ ID NO: 278 and SEQ ID NO: 285; SEQ ID NO: 278 and SEQ ID NO: 286; SEQ ID NO: 278 and SEQ ID NO: 287; SEQ ID NO: 278 and SEQ ID NO: 288; SEQ ID NO: 278 and SEQ ID NO: 289; SEQ ID NO: 278 and SEQ ID NO: 290; SEQ ID NO: 278 and SEQ ID NO: 291; SEQ ID NO: 278 and SEQ ID NO: 292; SEQ ID NO: 279 and SEQ ID NO: 280; SEQ ID NO: 279 and SEQ ID NO: 281; SEQ ID NO: 279 and SEQ ID NO: 282; SEQ ID NO: 279 and SEQ ID NO: 283; SEQ ID NO: 279 and SEQ ID NO: 284; SEQ ID NO: 279 and SEQ ID NO: 285; SEQ ID NO: 279 and SEQ ID NO: 286; SEQ ID NO: 279 and SEQ ID NO: 287; SEQ ID NO: 279 and SEQ ID NO: 288; SEQ ID NO: 279 and SEQ ID NO: 289; SEQ ID NO: 279 and SEQ ID NO: 290; SEQ ID NO: 279 and SEQ ID NO: 291; SEQ ID NO: 279 and SEQ ID NO: 292; SEQ ID NO: 280 and SEQ ID NO: 281; SEQ ID NO: 280 and SEQ ID NO: 282; SEQ ID NO: 280 and SEQ ID NO: 283; SEQ ID NO: 280 and SEQ ID NO: 284; SEQ ID NO: 280 and SEQ ID NO: 285; SEQ ID NO: 280 and SEQ ID NO: 286; SEQ ID NO: 280 and SEQ ID NO: 287; SEQ ID NO: 280 and SEQ ID NO: 288; SEQ ID NO: 280 and SEQ ID NO: 289; SEQ ID NO: 280 and SEQ ID NO: 290; SEQ ID NO: 280 and SEQ ID NO: 291; SEQ ID NO: 280 and SEQ ID NO: 292; SEQ ID NO: 281 and SEQ ID NO: 282; SEQ ID NO: 281 and SEQ ID NO: 283; SEQ ID NO: 281 and SEQ ID NO: 284; SEQ ID NO: 281 and SEQ ID NO: 285; SEQ ID NO: 281 and SEQ ID NO: 286; SEQ ID NO: 281 and SEQ ID NO: 287; SEQ ID NO: 281 and SEQ ID NO: 288; SEQ ID NO: 281 and SEQ ID NO: 289; SEQ ID NO: 281 and SEQ ID NO: 290; SEQ ID NO: 281 and SEQ ID NO: 291; SEQ ID NO: 281 and SEQ ID NO: 292; SEQ ID NO: 282 and SEQ ID NO: 283; SEQ ID NO: 282 and SEQ ID NO: 284; SEQ ID NO: 282 and SEQ ID NO: 285; SEQ ID NO: 282 and SEQ ID NO: 286; SEQ ID NO: 282 and SEQ ID NO: 287; SEQ ID NO: 282 and SEQ ID NO: 288; SEQ ID NO: 282 and SEQ ID NO: 289; SEQ ID NO: 282 and SEQ ID NO: 290; SEQ ID NO: 282 and SEQ ID NO: 291; SEQ ID NO: 282 and SEQ ID NO: 292; SEQ ID NO: 283 and SEQ ID NO: 284; SEQ ID NO: 283 and SEQ ID NO: 285; SEQ ID NO: 283 and SEQ ID NO: 286; SEQ ID NO: 283 and SEQ ID NO: 287; SEQ ID NO: 283 and SEQ ID NO: 288; SEQ ID NO: 283 and SEQ ID NO: 289; SEQ ID NO: 283 and SEQ ID NO: 290; SEQ ID NO: 283 and SEQ ID NO: 291; SEQ ID NO: 283 and SEQ ID NO: 292; SEQ ID NO: 284 and SEQ ID NO: 285; SEQ ID NO: 284 and SEQ ID NO: 286; SEQ ID NO: 284 and SEQ ID NO: 287; SEQ ID NO: 284 and SEQ ID NO: 288; SEQ ID NO: 284 and SEQ ID NO: 289; SEQ ID NO: 284 and SEQ ID NO: 290; SEQ ID NO: 284 and SEQ ID NO: 291; SEQ ID NO: 284 and SEQ ID NO: 292; SEQ ID NO: 285 and SEQ ID NO: 286; SEQ ID NO: 285 and SEQ ID NO: 287; SEQ ID NO: 285 and SEQ ID NO: 288; SEQ ID NO: 285 and SEQ ID NO: 289; SEQ ID NO: 285 and SEQ ID NO: 290; SEQ ID NO: 285 and SEQ ID NO: 291; SEQ ID NO: 285 and SEQ ID NO: 292; SEQ ID NO: 286 and SEQ ID NO: 287; SEQ ID NO: 286 and SEQ ID NO: 288; SEQ ID NO: 286 and SEQ ID NO: 289; SEQ ID NO: 286 and SEQ ID NO: 290; SEQ ID NO: 286 and SEQ ID NO: 291; SEQ ID NO: 286 and SEQ ID NO: 292; SEQ ID NO: 287 and SEQ ID NO: 288; SEQ ID NO: 287 and SEQ ID NO: 289; SEQ ID NO: 287 and SEQ ID NO: 290; SEQ ID NO: 287 and SEQ ID NO: 291; SEQ ID NO: 287 and SEQ ID NO: 292; SEQ ID NO: 288 and SEQ ID NO: 289; SEQ ID NO: 288 and SEQ ID NO: 290; SEQ ID NO: 288 and SEQ ID NO: 291; SEQ ID NO: 288 and SEQ ID NO: 292; SEQ ID NO: 289 and SEQ ID NO: 290; SEQ ID NO: 289 and SEQ ID NO: 291; SEQ ID NO: 289 and SEQ ID NO: 292; SEQ ID NO: 290 and SEQ ID NO: 291; SEQ ID NO: 290 and SEQ ID NO: 292; SEQ ID NO: 291 and SEQ ID NO: 292; SEQ ID NO: 270 and SEQ ID NO: 924; SEQ ID NO: 270 and SEQ ID NO: 925; SEQ ID NO: 270 and SEQ ID NO: 926; SEQ ID NO: 270 and SEQ ID NO: 927; SEQ ID NO: 270 and SEQ ID NO: 928; SEQ ID NO: 270 and SEQ ID NO: 929; SEQ ID NO: 270 and SEQ ID NO: 930; SEQ ID NO: 270 and SEQ ID NO: 931; SEQ ID NO: 270 and SEQ ID NO: 932; SEQ ID NO: 270 and SEQ ID NO: 933; SEQ ID NO: 270 and SEQ ID NO: 934; SEQ ID NO: 270 and SEQ ID NO: 935; SEQ ID NO: 270 and SEQ ID NO: 936; SEQ ID NO: 270 and SEQ ID NO: 937; SEQ ID NO: 270 and SEQ ID NO: 938; SEQ ID NO: 272 and SEQ ID NO: 924; SEQ ID NO: 272 and SEQ ID NO: 925; SEQ ID NO: 272 and SEQ ID NO: 926; SEQ ID NO: 272 and SEQ ID NO: 927; SEQ ID NO: 272 and SEQ ID NO: 928; SEQ ID NO: 272 and SEQ ID NO: 929; SEQ ID NO: 272 and SEQ ID NO: 930; SEQ ID NO: 272 and SEQ ID NO: 931; SEQ ID NO: 272 and SEQ ID NO: 932; SEQ ID NO: 272 and SEQ ID NO: 933; SEQ ID NO: 272 and SEQ ID NO: 934; SEQ ID NO: 272 and SEQ ID NO: 935; SEQ ID NO: 272 and SEQ ID NO: 936; SEQ ID NO: 272 and SEQ ID NO: 937; SEQ ID NO: 272 and SEQ ID NO: 938; SEQ ID NO: 273 and SEQ ID NO: 924; SEQ ID NO: 273 and SEQ ID NO: 925; SEQ ID NO: 273 and SEQ ID NO: 926; SEQ ID NO: 273 and SEQ ID NO: 927; SEQ ID NO: 273 and SEQ ID NO: 928; SEQ ID NO: 273 and SEQ ID NO: 929; SEQ ID NO: 273 and SEQ ID NO: 930; SEQ ID NO: 273 and SEQ ID NO: 931; SEQ ID NO: 273 and SEQ ID NO: 932; SEQ ID NO: 273 and SEQ ID NO: 933; SEQ ID NO: 273 and SEQ ID NO: 934; SEQ ID NO: 273 and SEQ ID NO: 935; SEQ ID NO: 273 and SEQ ID NO: 936; SEQ ID NO: 273 and SEQ ID NO: 937; SEQ ID NO: 273 and SEQ ID NO: 938; SEQ ID NO: 274 and SEQ ID NO: 924; SEQ ID NO: 274 and SEQ ID NO: 925; SEQ ID NO: 274 and SEQ ID NO: 926; SEQ ID NO: 274 and SEQ ID NO: 927; SEQ ID NO: 274 and SEQ ID NO: 928; SEQ ID NO: 274 and SEQ ID NO: 929; SEQ ID NO: 274 and SEQ ID NO: 930; SEQ ID NO: 274 and SEQ ID NO: 931; SEQ ID NO: 274 and SEQ ID NO: 932; SEQ ID NO: 274 and SEQ ID NO: 933; SEQ ID NO: 274 and SEQ ID NO: 934; SEQ ID NO: 274 and SEQ ID NO: 935; SEQ ID NO: 274 and SEQ ID NO: 936; SEQ ID NO: 274 and SEQ ID NO: 937; SEQ ID NO: 274 and SEQ ID NO: 938; SEQ ID NO: 275 and SEQ ID NO: 924; SEQ ID NO: 275 and SEQ ID NO: 925; SEQ ID NO: 275 and SEQ ID NO: 926; SEQ ID NO: 275 and SEQ ID NO: 927; SEQ ID NO: 275 and SEQ ID NO: 928; SEQ ID NO: 275 and SEQ ID NO: 929; SEQ ID NO: 275 and SEQ ID NO: 930; SEQ ID NO: 275 and SEQ ID NO: 931; SEQ ID NO: 275 and SEQ ID NO: 932; SEQ ID NO: 275 and SEQ ID NO: 933; SEQ ID NO: 275 and SEQ ID NO: 934; SEQ ID NO: 275 and SEQ ID NO: 935; SEQ ID NO: 275 and SEQ ID NO: 936; SEQ ID NO: 275 and SEQ ID NO: 937; SEQ ID NO: 275 and SEQ ID NO: 938; SEQ ID NO: 276 and SEQ ID NO: 924; SEQ ID NO: 276 and SEQ ID NO: 925; SEQ ID NO: 276 and SEQ ID NO: 926; SEQ ID NO: 276 and SEQ ID NO: 927; SEQ ID NO: 276 and SEQ ID NO: 928; SEQ ID NO: 276 and SEQ ID NO: 929; SEQ ID NO: 276 and SEQ ID NO: 930; SEQ ID NO: 276 and SEQ ID NO: 931; SEQ ID NO: 276 and SEQ ID NO: 932; SEQ ID NO: 276 and SEQ ID NO: 933; SEQ ID NO: 276 and SEQ ID NO: 934; SEQ ID NO: 276 and SEQ ID NO: 935; SEQ ID NO: 276 and SEQ ID NO: 936; SEQ ID NO: 276 and SEQ ID NO: 937; SEQ ID NO: 276 and SEQ ID NO: 938; SEQ ID NO: 277 and SEQ ID NO: 924; SEQ ID NO: 277 and SEQ ID NO: 925; SEQ ID NO: 277 and SEQ ID NO: 926; SEQ ID NO: 277 and SEQ ID NO: 927; SEQ ID NO: 277 and SEQ ID NO: 928; SEQ ID NO: 277 and SEQ ID NO: 929; SEQ ID NO: 277 and SEQ ID NO: 930; SEQ ID NO: 277 and SEQ ID NO: 931; SEQ ID NO: 277 and SEQ ID NO: 932; SEQ ID NO: 277 and SEQ ID NO: 933; SEQ ID NO: 277 and SEQ ID NO: 934; SEQ ID NO: 277 and SEQ ID NO: 935; SEQ ID NO: 277 and SEQ ID NO: 936; SEQ ID NO: 277 and SEQ ID NO: 937; SEQ ID NO: 277 and SEQ ID NO: 938; SEQ ID NO: 278 and SEQ ID NO: 924; SEQ ID NO: 278 and SEQ ID NO: 925; SEQ ID NO: 278 and SEQ ID NO: 926; SEQ ID NO: 278 and SEQ ID NO: 927; SEQ ID NO: 278 and SEQ ID NO: 928; SEQ ID NO: 278 and SEQ ID NO: 929; SEQ ID NO: 278 and SEQ ID NO: 930; SEQ ID NO: 278 and SEQ ID NO: 931; SEQ ID NO: 278 and SEQ ID NO: 932; SEQ ID NO: 278 and SEQ ID NO: 933; SEQ ID NO: 278 and SEQ ID NO: 934; SEQ ID NO: 278 and SEQ ID NO: 935; SEQ ID NO: 278 and SEQ ID NO: 936; SEQ ID NO: 278 and SEQ ID NO: 937; SEQ ID NO: 278 and SEQ ID NO: 938; SEQ ID NO: 279 and SEQ ID NO: 924; SEQ ID NO: 279 and SEQ ID NO: 925; SEQ ID NO: 279 and SEQ ID NO: 926; SEQ ID NO: 279 and SEQ ID NO: 927; SEQ ID NO: 279 and SEQ ID NO: 928; SEQ ID NO: 279 and SEQ ID NO: 929; SEQ ID NO: 279 and SEQ ID NO: 930; SEQ ID NO: 279 and SEQ ID NO: 931; SEQ ID NO: 279 and SEQ ID NO: 932; SEQ ID NO: 279 and SEQ ID NO: 933; SEQ ID NO: 279 and SEQ ID NO: 934; SEQ ID NO: 279 and SEQ ID NO: 935; SEQ ID NO: 279 and SEQ ID NO: 936; SEQ ID NO: 279 and SEQ ID NO: 937; SEQ ID NO: 279 and SEQ ID NO: 938; SEQ ID NO: 280 and SEQ ID NO: 924; SEQ ID NO: 280 and SEQ ID NO: 925; SEQ ID NO: 280 and SEQ ID NO: 926; SEQ ID NO: 280 and SEQ ID NO: 927; SEQ ID NO: 280 and SEQ ID NO: 928; SEQ ID NO: 280 and SEQ ID NO: 929; SEQ ID NO: 280 and SEQ ID NO: 930; SEQ ID NO: 280 and SEQ ID NO: 931; SEQ ID NO: 280 and SEQ ID NO: 932; SEQ ID NO: 280 and SEQ ID NO: 933; SEQ ID NO: 280 and SEQ ID NO: 934; SEQ ID NO: 280 and SEQ ID NO: 935; SEQ ID NO: 280 and SEQ ID NO: 936; SEQ ID NO: 280 and SEQ ID NO: 937; SEQ ID NO: 280 and SEQ ID NO: 938; SEQ ID NO: 281 and SEQ ID NO: 924; SEQ ID NO: 281 and SEQ ID NO: 925; SEQ ID NO: 281 and SEQ ID NO: 926; SEQ ID NO: 281 and SEQ ID NO: 927; SEQ ID NO: 281 and SEQ ID NO: 928; SEQ ID NO: 281 and SEQ ID NO: 929; SEQ ID NO: 281 and SEQ ID NO: 930; SEQ ID NO: 281 and SEQ ID NO: 931; SEQ ID NO: 281 and SEQ ID NO: 932; SEQ ID NO: 281 and SEQ ID NO: 933; SEQ ID NO: 281 and SEQ ID NO: 934; SEQ ID NO: 281 and SEQ ID NO: 935; SEQ ID NO: 281 and SEQ ID NO: 936; SEQ ID NO: 281 and SEQ ID NO: 937; SEQ ID NO: 281 and SEQ ID NO: 938; SEQ ID NO: 282 and SEQ ID NO: 924; SEQ ID NO: 282 and SEQ ID NO: 925; SEQ ID NO: 282 and SEQ ID NO: 926; SEQ ID NO: 282 and SEQ ID NO: 927; SEQ ID NO: 282 and SEQ ID NO: 928; SEQ ID NO: 282 and SEQ ID NO: 929; SEQ ID NO: 282 and SEQ ID NO: 930; SEQ ID NO: 282 and SEQ ID NO: 931; SEQ ID NO: 282 and SEQ ID NO: 932; SEQ ID NO: 282 and SEQ ID NO: 933; SEQ ID NO: 282 and SEQ ID NO: 934; SEQ ID NO: 282 and SEQ ID NO: 935; SEQ ID NO: 282 and SEQ ID NO: 936; SEQ ID NO: 282 and SEQ ID NO: 937; SEQ ID NO: 282 and SEQ ID NO: 938; SEQ ID NO: 283 and SEQ ID NO: 924; SEQ ID NO: 283 and SEQ ID NO: 925; SEQ ID NO: 283 and SEQ ID NO: 926; SEQ ID NO: 283 and SEQ ID NO: 927; SEQ ID NO: 283 and SEQ ID NO: 928; SEQ ID NO: 283 and SEQ ID NO: 929; SEQ ID NO: 283 and SEQ ID NO: 930; SEQ ID NO: 283 and SEQ ID NO: 931; SEQ ID NO: 283 and SEQ ID NO: 932; SEQ ID NO: 283 and SEQ ID NO: 933; SEQ ID NO: 283 and SEQ ID NO: 934; SEQ ID NO: 283 and SEQ ID NO: 935; SEQ ID NO: 283 and SEQ ID NO: 936; SEQ ID NO: 283 and SEQ ID NO: 937; SEQ ID NO: 283 and SEQ ID NO: 938; SEQ ID NO: 284 and SEQ ID NO: 924; SEQ ID NO: 284 and SEQ ID NO: 925; SEQ ID NO: 284 and SEQ ID NO: 926; SEQ ID NO: 284 and SEQ ID NO: 927; SEQ ID NO: 284 and SEQ ID NO: 928; SEQ ID NO: 284 and SEQ ID NO: 929; SEQ ID NO: 284 and SEQ ID NO: 930; SEQ ID NO: 284 and SEQ ID NO: 931; SEQ ID NO: 284 and SEQ ID NO: 932; SEQ ID NO: 284 and SEQ ID NO: 933; SEQ ID NO: 284 and SEQ ID NO: 934; SEQ ID NO: 284 and SEQ ID NO: 935; SEQ ID NO: 284 and SEQ ID NO: 936; SEQ ID NO: 284 and SEQ ID NO: 937; SEQ ID NO: 284 and SEQ ID NO: 938; SEQ ID NO: 285 and SEQ ID NO: 924; SEQ ID NO: 285 and SEQ ID NO: 925; SEQ ID NO: 285 and SEQ ID NO: 926; SEQ ID NO: 285 and SEQ ID NO: 927; SEQ ID NO: 285 and SEQ ID NO: 928; SEQ ID NO: 285 and SEQ ID NO: 929; SEQ ID NO: 285 and SEQ ID NO: 930; SEQ ID NO: 285 and SEQ ID NO: 931; SEQ ID NO: 285 and SEQ ID NO: 932; SEQ ID NO: 285 and SEQ ID NO: 933; SEQ ID NO: 285 and SEQ ID NO: 934; SEQ ID NO: 285 and SEQ ID NO: 935; SEQ ID NO: 285 and SEQ ID NO: 936; SEQ ID NO: 285 and SEQ ID NO: 937; SEQ ID NO: 285 and SEQ ID NO: 938; SEQ ID NO: 286 and SEQ ID NO: 924; SEQ ID NO: 286 and SEQ ID NO: 925; SEQ ID NO: 286 and SEQ ID NO: 926; SEQ ID NO: 286 and SEQ ID NO: 927; SEQ ID NO: 286 and SEQ ID NO: 928; SEQ ID NO: 286 and SEQ ID NO: 929; SEQ ID NO: 286 and SEQ ID NO: 930; SEQ ID NO: 286 and SEQ ID NO: 931; SEQ ID NO: 286 and SEQ ID NO: 932; SEQ ID NO: 286 and SEQ ID NO: 933; SEQ ID NO: 286 and SEQ ID NO: 934; SEQ ID NO: 286 and SEQ ID NO: 935; SEQ ID NO: 286 and SEQ ID NO: 936; SEQ ID NO: 286 and SEQ ID NO: 937; SEQ ID NO: 286 and SEQ ID NO: 938; SEQ ID NO: 287 and SEQ ID NO: 924; SEQ ID NO: 287 and SEQ ID NO: 925; SEQ ID NO: 287 and SEQ ID NO: 926; SEQ ID NO: 287 and SEQ ID NO: 927; SEQ ID NO: 287 and SEQ ID NO: 928; SEQ ID NO: 287 and SEQ ID NO: 929; SEQ ID NO: 287 and SEQ ID NO: 930; SEQ ID NO: 287 and SEQ ID NO: 931; SEQ ID NO: 287 and SEQ ID NO: 932; SEQ ID NO: 287 and SEQ ID NO: 933; SEQ ID NO: 287 and SEQ ID NO: 934; SEQ ID NO: 287 and SEQ ID NO: 935; SEQ ID NO: 287 and SEQ ID NO: 936; SEQ ID NO: 287 and SEQ ID NO: 937; SEQ ID NO: 287 and SEQ ID NO: 938; SEQ ID NO: 288 and SEQ ID NO: 924; SEQ ID NO: 288 and SEQ ID NO: 925; SEQ ID NO: 288 and SEQ ID NO: 926; SEQ ID NO: 288 and SEQ ID NO: 927; SEQ ID NO: 288 and SEQ ID NO: 928; SEQ ID NO: 288 and SEQ ID NO: 929; SEQ ID NO: 288 and SEQ ID NO: 930; SEQ ID NO: 288 and SEQ ID NO: 931; SEQ ID NO: 288 and SEQ ID NO: 932; SEQ ID NO: 288 and SEQ ID NO: 933; SEQ ID NO: 288 and SEQ ID NO: 934; SEQ ID NO: 288 and SEQ ID NO: 935; SEQ ID NO: 288 and SEQ ID NO: 936; SEQ ID NO: 288 and SEQ ID NO: 937; SEQ ID NO: 288 and SEQ ID NO: 938; SEQ ID NO: 289 and SEQ ID NO: 924; SEQ ID NO: 289 and SEQ ID NO: 925; SEQ ID NO: 289 and SEQ ID NO: 926; SEQ ID NO: 289 and SEQ ID NO: 927; SEQ ID NO: 289 and SEQ ID NO: 928; SEQ ID NO: 289 and SEQ ID NO: 929; SEQ ID NO: 289 and SEQ ID NO: 930; SEQ ID NO: 289 and SEQ ID NO: 931; SEQ ID NO: 289 and SEQ ID NO: 932; SEQ ID NO: 289 and SEQ ID NO: 933; SEQ ID NO: 289 and SEQ ID NO: 934; SEQ ID NO: 289 and SEQ ID NO: 935; SEQ ID NO: 289 and SEQ ID NO: 936; SEQ ID NO: 289 and SEQ ID NO: 937; SEQ ID NO: 289 and SEQ ID NO: 938; SEQ ID NO: 290 and SEQ ID NO: 924; SEQ ID NO: 290 and SEQ ID NO: 925; SEQ ID NO: 290 and SEQ ID NO: 926; SEQ ID NO: 290 and SEQ ID NO: 927; SEQ ID NO: 290 and SEQ ID NO: 928; SEQ ID NO: 290 and SEQ ID NO: 929; SEQ ID NO: 290 and SEQ ID NO: 930; SEQ ID NO: 290 and SEQ ID NO: 931; SEQ ID NO: 290 and SEQ ID NO: 932; SEQ ID NO: 290 and SEQ ID NO: 933; SEQ ID NO: 290 and SEQ ID NO: 934; SEQ ID NO: 290 and SEQ ID NO: 935; SEQ ID NO: 290 and SEQ ID NO: 936; SEQ ID NO: 290 and SEQ ID NO: 937; SEQ ID NO: 290 and SEQ ID NO: 938; SEQ ID NO: 291 and SEQ ID NO: 924; SEQ ID NO: 291 and SEQ ID NO: 925; SEQ ID NO: 291 and SEQ ID NO: 926; SEQ ID NO: 291 and SEQ ID NO: 927; SEQ ID NO: 291 and SEQ ID NO: 928; SEQ ID NO: 291 and SEQ ID NO: 929; SEQ ID NO: 291 and SEQ ID NO: 930; SEQ ID NO: 291 and SEQ ID NO: 931; SEQ ID NO: 291 and SEQ ID NO: 932; SEQ ID NO: 291 and SEQ ID NO: 933; SEQ ID NO: 291 and SEQ ID NO: 934; SEQ ID NO: 291 and SEQ ID NO: 935; SEQ ID NO: 291 and SEQ ID NO: 936; SEQ ID NO: 291 and SEQ ID NO: 937; SEQ ID NO: 291 and SEQ ID NO: 938; SEQ ID NO: 292 and SEQ ID NO: 924; SEQ ID NO: 292 and SEQ ID NO: 925; SEQ ID NO: 292 and SEQ ID NO: 926; SEQ ID NO: 292 and SEQ ID NO: 927; SEQ ID NO: 292 and SEQ ID NO: 928; SEQ ID NO: 292 and SEQ ID NO: 929; SEQ ID NO: 292 and SEQ ID NO: 930; SEQ ID NO: 292 and SEQ ID NO: 931; SEQ ID NO: 292 and SEQ ID NO: 932; SEQ ID NO: 292 and SEQ ID NO: 933; SEQ ID NO: 292 and SEQ ID NO: 934; SEQ ID NO: 292 and SEQ ID NO: 935; SEQ ID NO: 292 and SEQ ID NO: 936; SEQ ID NO: 292 and SEQ ID NO: 937; SEQ ID NO: 292 and SEQ ID NO: 938; SEQ ID NO: 950 and SEQ ID NO: 275; SEQ ID NO: 951 and SEQ ID NO: 275; SEQ ID NO: 952 and SEQ ID NO: 275; SEQ ID NO: 953 and SEQ ID NO: 275; SEQ ID NO: 954 and SEQ ID NO: 275; or SEQ ID NO: 955 and SEQ ID NO: 275; and a nucleic acid encoding a Staphylococcus lugdunensis (SluCas9), wherein the nucleic acid encoding the SluCas9 may be on the same or different nucleic acid molecule as the pair of guide RNAs.

In some embodiments, a method for treating DMD is provided comprising administering a composition comprising one or more guide RNAs wherein each guide RNA comprises a guide sequence of Table 6, or comprises a guide sequence comprising at least 16, 17, 18, 19, or 20 contiguous nucleotides of a guide sequence of Table 6, or is at least 90% identical to guide sequence selected from Table 6. In each instance, the composition may comprise a nucleic acid encoding the guide RNA(s). In some embodiments, a method for treating DMD is provided comprising administering a composition comprising one or more nucleic acid molecules encoding one or more guide RNAs, wherein each guide RNA comprises a guide sequence of Table 6, or at least 16, 17, 18, 19, or 20 contiguous nucleotides of a guide sequence of Table 6, or is at least 90% identical to guide sequence selected from Table 6.

In some embodiments, a method for treating DMD is provided comprising administering a composition comprising a guide RNA wherein the guide RNA comprises a guide sequence of Table 6, or at least 16, 17, 18, 19, or 20 contiguous nucleotides of a guide sequence of Table 6, or is at least 90% identical to guide sequence of Table 6. In each instance, the composition may comprise a nucleic acid encoding the guide RNA.

In some embodiments, a method for treating DMD is provided comprising administering a composition comprising a guide RNA comprising no more than 16, no more than 17, no more than 18, no more than 19, no more than 20, no more than 21, or no more than 22 contiguous nucleotides of a guide sequence of Table 6, or is at least 90% identical to no more than 16, no more than 17, no more than 18, no more than 19, no more than 20, no more than 21, or no more than 22 contiguous nucleotides of a guide sequence of Table 6. In each instance, the composition may comprise a nucleic acid encoding the guide RNA.

In some embodiments, a method for treating DMD is provided comprising administering a composition comprising two or more guide RNAs wherein each guide RNA comprises a guide sequence of Table 6, or at least 16, 17, 18, 19, or 20 contiguous nucleotides of a guide sequence of Table 6, or is at least 90% identical to guide sequence selected from Table 6. In each instance, the composition may comprise a nucleic acid encoding the guide RNA.

In some embodiments, a method for treating DMD is provided comprising administering a composition comprising two or more guide RNAs wherein at least one of the two or more guide RNAs, optionally each guide RNA, comprises no more than 16, no more than 17, no more than 18, no more than 19, no more than 20, no more than 21, or no more than 22 contiguous nucleotides of a guide sequence of Table 6, is at least 90% identical to no more than 16, no more than 17, no more than 18, no more than 19, no more than 20, no more than 21, or no more than 22 contiguous nucleotides of a guide sequence selected from Table 6. In each instance, the composition may comprise a nucleic acid encoding the guide RNA.

In some embodiments, a method for treating DMD is provided comprising administering a composition comprising a SaCas9 or SluCas9 or one or more nucleic acid molecules encoding a SaCas9 or SluCas9 and a pair of guide RNAs comprising a first guide sequence and a second guide sequence, wherein the first and second guide sequence are selected from the guide sequence pairs of:
a. For exon 44 (with e.g., SaCas9), SEQ ID NOs: 1 and 3; 110 and 120; 110 and 121; 110 and 122; 110 and 123; 110 and 124; 110 and 125; 111 and 112; 111 and 113; 111 and 114; 111 and 115; 111 and 116; 111 and 117; 111 and 118; 111 and 119; 111 and 120; 111 and 121; 111 and 122; 111 and 123; 111 and 124; 111 and 125; 112 and 113; 112 and 114; 112 and 115; 112 and 116; 112 and 117; 112 and 118; 112 and 119; 112 and 120; 112 and 121; 112 and 122; 112 and 123; 112 and 124; 112 and 125; 113 and 114; 113 and 115; 113 and 116; 113 and 117; 113 and 118; 113 and 119; 113 and 120; 113 and 121; 113 and 122; 113 and 123; 113 and 124; 113 and 125; 114 and 115; 114 and 116; 114 and 117; 114 and 118; 114 and 119; 114 and 120; 114 and 121; 114 and 122; 114 and 123; 114 and 124; 114 and 125; 115 and 116; 115 and 117; 115 and 118; 115 and 119; 115 and 120; 115 and 121; 115 and 122; 115 and 123; 115 and 124; 115 and 125; 116 and 117; 116 and 118; 116 and 119; 116 and 120; 116 and 121; 116 and 122; 116 and 123; 116 and 124; 116 and 125; 117 and 118; 117 and 119; 117 and 120; 117 and 121; 117 and 122; 117 and 123; 117 and 124; 117 and 125; 118 and 119; 118 and 120; 118 and 121; 118 and 122; 118 and 123; 118 and 124; 118 and 125; 119 and 120; 119 and 121; 119 and 122; 119 and 123; 119 and 124; 119 and 125; 120 and 121; 120 and 122; 120 and 123; 120 and 124; 120 and 125; 121 and 122; 121 and 123; 121 and 124; 121 and 125; 122 and 123; 122 and 124; 122 and 125; 123 and 124; 123 and 125; 124 and 125;
b. For exon 44 (with, e.g., SluCas9), SEQ ID NOs: 200 and 201; 200 and 202; 200 and 203; 200 and 204; 200 and 205; 200 and 206; 200 and 207; 200 and 208; 201 and 202; 201 and 203; 201 and 204; 201 and 205; 201 and 206; 201 and 207; 201 and 208; 202 and 203; 202 and 204; 202 and 205; 202 and 206; 202 and 207; 202 and 208; 203 and 204; 203 and 205; 203 and 206; 203 and 207; 203 and 208; 204 and 205; 204 and 206; 204 and 207; 204 and 208; 205 and 206; 205 and 207; 205 and 208; 206 and 207; 206 and 208; 207 and 208;
c. For exon 45 (with, e.g., SaCas9), SEQ ID NOs: 3 and 4; 3 and 5; 3 and 6; 3 and 7; 3 and 8; 3 and 9; 4 and 5; 4 and 6; 4 and 7; 4 and 8; 4 and 9; 5 and 6; 5 and 7; 5 and 8; 5 and 9; 6 and 7; 6 and 8; 6 and 9; 7 and 8; 7 and 9; 8 and 9; 126 and 127; 126 and 128; 126 and 129; 126 and 130; 126 and 131; 126 and 132; 126 and 133; 126 and 134; 126 and 135; 126 and 136; 126 and 137; 126 and 138; 126 and 139; 126 and 140; 126 and 141; 126 and 142; 126 and 143; 126 and 144; 126 and 145; 126 and 146; 126 and 147; 127 and 128; 127 and 129; 127 and 130; 127 and 131; 127 and 132; 127 and 133; 127 and 134; 127 and 135; 127 and 136; 127 and 137; 127 and 138; 127 and 139; 127 and 140; 127 and 141; 127 and 142; 127 and 143; 127 and 144; 127 and 145; 127 and 146; 127 and 147; 128 and 129; 128 and 130; 128 and 131; 128 and 132; 128 and 133; 128 and 134; 128 and 135; 128 and 136; 128 and 137; 128 and 138; 128 and 139; 128 and 140; 128 and 141; 128 and 142; 128 and 143; 128 and 144; 128 and 145; 128 and 146; 128 and 147; 129 and 130; 129 and 131; 129 and 132; 129 and 133; 129 and 134; 129 and 135; 129 and 136; 129 and 137; 129 and 138; 129 and 139; 129 and 140; 129 and 141; 129 and 142; 129 and 143; 129 and 144; 129 and 145; 129 and 146; 129 and 147; 130 and 131; 130 and 132; 130 and 133; 130 and 134; 130 and 135; 130 and 136; 130 and 137; 130 and 138; 130 and 139; 130 and 140; 130 and 141; 130 and 142; 130 and 143; 130 and 144; 130 and 145; 130 and 146; 130 and 147; 131 and 132; 131 and 133; 131 and 134; 131 and 135; 131 and 136; 131 and 137; 131 and 138; 131 and 139; 131 and 140; 131 and 141; 131 and 142; 131 and 143; 131 and 144; 131 and 145; 131 and 146; 131 and 147; 132 and 133; 132 and 134; 132 and 135; 132 and 136; 132 and 137; 132 and 138; 132 and 139; 132 and 140; 132 and 141; 132 and 142; 132 and 143; 132 and 144; 132 and 145; 132 and 146; 132 and 147; 133 and 134; 133 and 135; 133 and 136; 133 and 137; 133 and 138; 133 and 139; 133 and 140; 133 and 141; 133 and 142; 133 and 143; 133 and 144; 133 and 145; 133 and 146; 133 and 147; 134 and 135; 134 and 136; 134 and 137; 134 and 138; 134 and 139; 134 and 140; 134 and 141; 134 and 142; 134 and 143; 134 and 144; 134 and 145; 134 and 146; 134 and 147; 135 and 136; 135 and 137; 135 and 138; 135 and 139; 135 and 140; 135 and 141; 135 and 142; 135 and 143; 135 and 144; 135 and 145; 135 and 146; 135 and 147; 136 and 137; 136 and 138; 136 and 139; 136 and 140; 136 and 141; 136 and 142; 136 and 143; 136 and 144; 136 and 145; 136 and 146; 136 and 147; 137 and 138; 137 and 139; 137 and 140; 137 and 141; 137 and 142; 137 and 143; 137 and 144; 137 and 145; 137 and 146; 137 and 147; 138 and 139; 138 and 140; 138 and 141; 138 and 142; 138 and 143; 138 and 144; 138 and 145; 138 and 146; 138 and 147; 139 and 140; 139 and 141; 139 and 142; 139 and 143; 139 and 144; 139 and 145; 139 and 146; 139 and 147; 140 and 141; 140 and 142; 140 and 143; 140 and 144; 140 and 145; 140 and 146; 140 and 147; 141 and 142; 141 and 143; 141 and 144; 141 and 145; 141 and 146; 141 and 147; 142 and 143; 142 and 144; 142 and 145; 142 and 146; 142 and 147; 143 and 144; 143 and 145; 143 and 146; 143 and 147; 144 and 145; 144 and 146; 144 and 147; 145 and 146; 145 and 147; 146 and 147;
d. For exon 45 (with, e.g., SluCas9), SEQ ID NOs: 209 and 210; 209 and 211; 209 and 212; 209 and 213; 209 and 214; 209 and 215; 209 and 216; 209 and 217; 209 and 218; 209 and 219; 209 and 220; 209 and 221; 209 and 222; 209 and 223; 209 and 224; 209 and 225; 209 and 226; 209 and 227; 209 and 228; 209 and 229; 209 and 230; 210 and 211; 210 and 212; 210 and 213; 210 and 214; 210 and 215; 210 and 216; 210 and 217; 210 and 218; 210 and 219; 210 and 220; 210 and 221; 210 and 222; 210 and 223; 210 and 224; 210 and 225; 210 and 226; 210 and 227; 210 and 228; 210 and 229; 210 and 230; 211 and 212; 211 and 213; 211 and 214; 211 and 215; 211 and 216; 211 and 217; 211 and 218; 211 and 219; 211 and 220; 211 and 221; 211 and 222; 211 and 223; 211 and 224; 211 and 225; 211 and 226; 211 and 227; 211 and 228; 211 and 229; 211 and 230; 212 and 213; 212 and 214; 212 and 215; 212 and 216; 212 and 217; 212 and 218; 212 and 219; 212 and 220; 212 and 221; 212 and 222; 212 and 223; 212 and 224; 212 and 225; 212 and 226; 212 and 227; 212 and 228; 212 and 229; 212 and 230; 213 and 214; 213 and 215; 213 and 216; 213 and 217; 213 and 218; 213 and 219; 213 and 220; 213 and 221; 213 and 222; 213 and 223; 213 and 224; 213 and 225; 213 and 226; 213 and 227; 213 and 228; 213 and 229; 213 and 230; 214 and 215; 214 and 216; 214 and 217; 214 and 218; 214 and 219; 214 and 220; 214 and 221; 214 and 222; 214 and 223; 214 and 224; 214 and 225; 214 and 226; 214 and 227; 214 and 228; 214 and 229; 214 and 230; 215 and 216; 215 and 217; 215 and 218; 215 and 219; 215 and 220; 215 and 221; 215 and 222; 215 and 223; 215 and 224; 215 and 225; 215 and 226; 215 and 227; 215 and 228; 215 and 229; 215 and 230; 216 and 217; 216 and 218; 216 and 219; 216 and 220; 216 and 221; 216 and 222; 216 and 223; 216 and 224; 216 and 225; 216 and 226; 216 and 227; 216 and 228; 216 and 229; 216 and 230; 217 and 218; 217 and 219; 217 and 220; 217 and 221; 217 and 222; 217 and 223; 217 and 224; 217 and 225; 217 and 226; 217 and 227; 217 and 228; 217 and 229; 217 and 230; 218 and 219; 218 and 220; 218 and 221; 218 and 222; 218 and 223; 218 and 224; 218 and 225; 218 and 226; 218 and 227; 218 and 228; 218 and 229; 218 and 230; 219 and 220; 219 and 221; 219 and 222; 219 and 223; 219 and 224; 219 and 225; 219 and 226; 219 and 227; 219 and 228; 219 and 229; 219 and 230; 220 and 221; 220 and 222; 220 and 223; 220 and 224; 220 and 225; 220 and 226; 220 and 227; 220 and 228; 220 and 229; 220 and 230; 221 and 222; 221 and 223; 221 and 224; 221 and 225; 221 and 226; 221 and 227; 221 and 228; 221 and 229; 221 and 230; 222 and 223; 222 and 224; 222 and 225; 222 and 226; 222 and 227; 222 and 228; 222 and 229; 222 and 230; 223 and 224; 223 and 225; 223 and 226; 223 and 227; 223 and 228; 223 and 229; 223 and 230; 224 and 225; 224 and 226; 224 and 227; 224 and 228; 224 and 229; 224 and 230; 225 and 226; 225 and 227; 225 and 228; 225 and 229; 225 and 230; 226 and 227; 226 and 228; 226 and 229; 226 and 230; 227 and 228; 227 and 229; 227 and 230; 228 and 229; 228 and 230; 229 and 230;
e. For exon 50 (with, e.g., SaCas9), SEQ ID NOs: 148 and 149; 148 and 150; 148 and 151; 148 and 152; 148 and 153; 148 and 154; 148 and 155; 148 and 156; 148 and 157; 148 and 158; 148 and 159; 149 and 150; 149 and 151; 149 and 152; 149 and 153; 149 and 154; 149 and 155; 149 and 156; 149 and 157; 149 and 158; 149 and 159; 150 and 151; 150 and 152; 150 and 153; 150 and 154; 150 and 155; 150 and 156; 150 and 157; 150 and 158; 150 and 159; 151 and 152; 151 and 153; 151 and 154; 151 and 155; 151 and 156; 151 and 157; 151 and 158; 151 and 159; 152 and 153; 152 and 154; 152 and 155; 152 and 156; 152 and 157; 152 and 158; 152 and 159; 153 and 154; 153 and 155; 153 and 156; 153 and 157; 153 and 158; 153 and 159; 154 and 155; 154 and 156; 154 and 157; 154 and 158; 154 and 159; 155 and 156; 155 and 157; 155 and 158; 155 and 159; 156 and 157; 156 and 158; 156 and 159; 157 and 158; 157 and 159; 158 and 159;
f. For exon 50 (with, e.g., SluCas9), SEQ ID NOs: 231 and 232; 231 and 233; 231 and 234; 231 and 235; 231 and 236; 231 and 237; 231 and 238; 231 and 239; 231 and 240; 231 and 241; 231 and 242; 232 and 233; 232 and 234; 232 and 235; 232 and 236; 232 and 237; 232 and 238; 232 and 239; 232 and 240; 232 and 241; 232 and 242; 233 and 234; 233 and 235; 233 and 236; 233 and 237; 233 and 238; 233 and 239; 233 and 240; 233 and 241; 233 and 242; 234 and 235; 234 and 236; 234 and 237; 234 and 238; 234 and 239; 234 and 240; 234 and 241; 234 and 242; 235 and 236; 235 and 237; 235 and 238; 235 and 239; 235 and 240; 235 and 241; 235 and 242; 236 and 237; 236 and 238; 236 and 239; 236 and 240; 236 and 241; 236 and 242; 237 and 238; 237 and 239; 237 and 240; 237 and 241; 237 and 242; 238 and 239; 238 and 240; 238 and 241; 238 and 242; 239 and 240; 239 and 241; 239 and 242; 240 and 241; 240 and 242; 241 and 242;
g. For exon 51 (with, e.g., SaCas9), SEQ ID NOs: 11 and 12; 11 and 13; 11 and 14; 11 and 15; 12 and 13; 12 and 14; 12 and 15; 13 and 14; 13 and 15; 14 and 15; 27 and 28; 27 and 29; 27 and 30; 27 and 31; 27 and 32; 27 and 33; 27 and 34; 27 and 35; 27 and 36; 27 and 37; 27 and 38; 27 and 39; 27 and 40; 27 and 41; 27 and 42; 27 and 43; 27 and 44; 27 and 45; 27 and 46; 27 and 47; 27 and 48; 27 and 53; 27 and 54; 27 and 55; 27 and 56; 27 and 57; 27 and 58; 27 and 59; 27 and 60; 27 and 61; 27 and 62; 27 and 63; 27 and 64; 27 and 65; 27 and 66; 27 and 67; 27 and 68; 27 and 69; 28 and 29; 28 and 30; 28 and 31; 28 and 32; 28 and 33; 28 and 34; 28 and 35; 28 and 36; 28 and 37; 28 and 38; 28 and 39; 28 and 40; 28 and 41; 28 and 42; 28 and 43; 28 and 44; 28 and 45; 28 and 46; 28 and 47; 28 and 48; 28 and 53; 28 and 54; 28 and 55; 28 and 56; 28 and 57; 28 and 58; 28 and 59; 28 and 60; 28 and 61; 28 and 62; 28 and 63; 28 and 64; 28 and 65; 28 and 66; 28 and 67; 28 and 68; 28 and 69; 29 and 30; 29 and 31; 29 and 32; 29 and 33; 29 and 34; 29 and 35; 29 and 36; 29 and 37; 29 and 38; 29 and 39; 29 and 40; 29 and 41; 29 and 42; 29 and 43; 29 and 44; 29 and 45; 29 and 46; 29 and 47; 29 and 48; 29 and 53; 29 and 54; 29 and 55; 29 and 56; 29 and 57; 29 and 58; 29 and 59; 29 and 60; 29 and 61; 29 and 62; 29 and 63; 29 and 64; 29 and 65; 29 and 66; 29 and 67; 29 and 68; 29 and 69; 30 and 31; 30 and 32; 30 and 33; 30 and 34; 30 and 35; 30 and 36; 30 and 37; 30 and 38; 30 and 39; 30 and 40; 30 and 41; 30 and 42; 30 and 43; 30 and 44; 30 and 45; 30 and 46; 30 and 47; 30 and 48; 30 and 53; 30 and 54; 30 and 55; 30 and 56; 30 and 57; 30 and 58; 30 and 59; 30 and 60; 30 and 61; 30 and 62; 30 and 63; 30 and 64; 30 and 65; 30 and 66; 30 and 67; 30 and 68; 30 and 69; 31 and 32; 31 and 33; 31 and 34; 31 and 35; 31 and 36; 31 and 37; 31 and 38; 31 and 39; 31 and 40; 31 and 41; 31 and 42; 31 and 43; 31 and 44; 31 and 45; 31 and 46; 31 and 47; 31 and 48; 31 and 53; 31 and 54; 31 and 55; 31 and 56; 31 and 57; 31 and 58; 31 and 59; 31 and 60; 31 and 61; 31 and 62; 31 and 63; 31 and 64; 31 and 65; 31 and 66; 31 and 67; 31 and 68; 31 and 69; 32 and 33; 32 and 34; 32 and 35; 32 and 36; 32 and 37; 32 and 38; 32 and 39; 32 and 40; 32 and 41; 32 and 42; 32 and 43; 32 and 44; 32 and 45; 32 and 46; 32 and 47; 32 and 48; 32 and 53; 32 and 54; 32 and 55; 32 and 56; 32 and 57; 32 and 58; 32 and 59; 32 and 60; 32 and 61; 32 and 62; 32 and 63; 32 and 64; 32 and 65; 32 and 66; 32 and 67; 32 and 68; 32 and 69; 33 and 34; 33 and 35; 33 and 36; 33 and 37; 33 and 38; 33 and 39; 33 and 40; 33 and 41; 33 and 42; 33 and 43; 33 and 44; 33 and 45; 33 and 46; 33 and 47; 33 and 48; 33 and 53; 33 and 54; 33 and 55; 33 and 56; 33 and 57; 33 and 58; 33 and 59; 33 and 60; 33 and 61; 33 and 62; 33 and 63; 33 and 64; 33 and 65; 33 and 66; 33 and 67; 33 and 68; 33 and 69; 34 and 35; 34 and 36; 34 and 37; 34 and 38; 34 and 39; 34 and 40; 34 and 41; 34 and 42; 34 and 43; 34 and 44; 34 and 45; 34 and 46; 34 and 47; 34 and 48; 34 and 53; 34 and 54; 34 and 55; 34 and 56; 34 and 57; 34 and 58; 34 and 59; 34 and 60; 34 and 61; 34 and 62; 34 and 63; 34 and 64; 34 and 65; 34 and 66; 34 and 67; 34 and 68; 34 and 69; 35 and 36; 35 and 37; 35 and 38; 35 and 39; 35 and 40; 35 and 41; 35 and 42; 35 and 43; 35 and 44; 35 and 45; 35 and 46; 35 and 47; 35 and 48; 35 and 53; 35 and 54; 35 and 55; 35 and 56; 35 and 57; 35 and 58; 35 and 59; 35 and 60; 35 and 61; 35 and 62; 35 and 63; 35 and 64; 35 and 65; 35 and 66; 35 and 67; 35 and 68; 35 and 69; 36 and 37; 36 and 38; 36 and 39; 36 and 40; 36 and 41; 36 and 42; 36 and 43; 36 and 44; 36 and 45; 36 and 46; 36 and 47; 36 and 48; 36 and 53; 36 and 54; 36 and 55; 36 and 56; 36 and 57; 36 and 58; 36 and 59; 36 and 60; 36 and 61; 36 and 62; 36 and 63; 36 and 64; 36 and 65; 36 and 66; 36 and 67; 36 and 68; 36 and 69; 37 and 38; 37 and 39; 37 and 40; 37 and 41; 37 and 42; 37 and 43; 37 and 44; 37 and 45; 37 and 46; 37 and 47; 37 and 48; 37 and 53; 37 and 54; 37 and 55; 37 and 56; 37 and 57; 37 and 58; 37 and 59; 37 and 60; 37 and 61; 37 and 62; 37 and 63; 37 and 64; 37 and 65; 37 and 66; 37 and 67; 37 and 68; 37 and 69; 38 and 39; 38 and 40; 38 and 41; 38 and 42; 38 and 43; 38 and 44; 38 and 45; 38 and 46; 38 and 47; 38 and 48; 38 and 53; 38 and 54; 38 and 55; 38 and 56; 38 and 57; 38 and 58; 38 and 59; 38 and 60; 38 and 61; 38 and 62; 38 and 63; 38 and 64; 38 and 65; 38 and 66; 38 and 67; 38 and 68; 38 and 69; 39 and 40; 39 and 41; 39 and 42; 39 and 43; 39 and 44; 39 and 45; 39 and 46; 39 and 47; 39 and 48; 39 and 53; 39 and 54; 39 and 55; 39 and 56; 39 and 57; 39 and 58; 39 and 59; 39 and 60; 39 and 61; 39 and 62; 39 and 63; 39 and 64; 39 and 65; 39 and 66; 39 and 67; 39 and 68; 39 and 69; 40 and 41; 40 and 42; 40 and 43; 40 and 44; 40 and 45; 40 and 46; 40 and 47; 40 and 48; 40 and 53; 40 and 54; 40 and 55; 40 and 56; 40 and 57; 40 and 58; 40 and 59; 40 and 60; 40 and 61; 40 and 62; 40 and 63; 40 and 64; 40 and 65; 40 and 66; 40 and 67; 40 and 68; 40 and 69; 41 and 42; 41 and 43; 41 and 44; 41 and 45; 41 and 46; 41 and 47; 41 and 48; 41 and 53; 41 and 54; 41 and 55; 41 and 56; 41 and 57; 41 and 58; 41 and 59; 41 and 60; 41 and 61; 41 and 62; 41 and 63; 41 and 64; 41 and 65; 41 and 66; 41 and 67; 41 and 68; 41 and 69; 42 and 43; 42 and 44; 42 and 45; 42 and 46; 42 and 47; 42 and 48; 42 and 53; 42 and 54; 42 and 55; 42 and 56; 42 and 57; 42 and 58; 42 and 59; 42 and 60; 42 and 61; 42 and 62; 42 and 63; 42 and 64; 42 and 65; 42 and 66; 42 and 67; 42 and 68; 42 and 69; 43 and 44; 43 and 45; 43 and 46; 43 and 47; 43 and 48; 43 and 53; 43 and 54; 43 and 55; 43 and 56; 43 and 57; 43 and 58; 43 and 59; 43 and 60; 43 and 61; 43 and 62; 43 and 63; 43 and 64; 43 and 65; 43 and 66; 43 and 67; 43 and 68; 43 and 69; 44 and 45; 44 and 46; 44 and 47; 44 and 48; 44 and 53; 44 and 54; 44 and 55; 44 and 56; 44 and 57; 44 and 58; 44 and 59; 44 and 60; 44 and 61; 44 and 62; 44 and 63; 44 and 64; 44 and 65; 44 and 66; 44 and 67; 44 and 68; 44 and 69; 45 and 46; 45 and 47; 45 and 48; 45 and 53; 45 and 54; 45 and 55; 45 and 56; 45 and 57; 45 and 58; 45 and 59; 45 and 60; 45 and 61; 45 and 62; 45 and 63; 45 and 64; 45 and 65; 45 and 66; 45 and 67; 45 and 68; 45 and 69; 46 and 47; 46 and 48; 46 and 53; 46 and 54; 46 and 55; 46 and 56; 46 and 57; 46 and 58; 46 and 59; 46 and 60; 46 and 61; 46 and 62; 46 and 63; 46 and 64; 46 and 65; 46 and 66; 46 and 67; 46 and 68; 46 and 69; 47 and 48; 47 and 53; 47 and 54; 47 and 55; 47 and 56; 47 and 57; 47 and 58; 47 and 59; 47 and 60; 47 and 61; 47 and 62; 47 and 63; 47 and 64; 47 and 65; 47 and 66; 47 and 67; 47 and 68; 47 and 69; 48 and 53; 48 and 54; 48 and 55; 48 and 56; 48 and 57; 48 and 58; 48 and 59; 48 and 60; 48 and 61; 48 and 62; 48 and 63; 48 and 64; 48 and 65; 48 and 66; 48 and 67; 48 and 68; 48 and 69; 53 and 54; 53 and 55; 53 and 56; 53 and 57; 53 and 58; 53 and 59; 53 and 60; 53 and 61; 53 and 62; 53 and 63; 53 and 64; 53 and 65; 53 and 66; 53 and 67; 53 and 68; 53 and 69; 54 and 55; 54 and 56; 54 and 57; 54 and 58; 54 and 59; 54 and 60; 54 and 61; 54 and 62; 54 and 63; 54 and 64; 54 and 65; 54 and 66; 54 and 67; 54 and 68; 54 and 69; 55 and 56; 55 and 57; 55 and 58; 55 and 59; 55 and 60; 55 and 61; 55 and 62; 55 and 63; 55 and 64; 55 and 65; 55 and 66; 55 and 67; 55 and 68; 55 and 69; 56 and 57; 56 and 58; 56 and 59; 56 and 60; 56 and 61; 56 and 62; 56 and 63; 56 and 64; 56 and 65; 56 and 66; 56 and 67; 56 and 68; 56 and 69; 57 and 58; 57 and 59; 57 and 60; 57 and 61; 57 and 62; 57 and 63; 57 and 64; 57 and 65; 57 and 66; 57 and 67; 57 and 68; 57 and 69; 58 and 59; 58 and 60; 58 and 61; 58 and 62; 58 and 63; 58 and 64; 58 and 65; 58 and 66; 58 and 67; 58 and 68; 58 and 69; 59 and 60; 59 and 61; 59 and 62; 59 and 63; 59 and 64; 59 and 65; 59 and 66; 59 and 67; 59 and 68; 59 and 69; 60 and 61; 60 and 62; 60 and 63; 60 and 64; 60 and 65; 60 and 66; 60 and 67; 60 and 68; 60 and 69; 61 and 62; 61 and 63; 61 and 64; 61 and 65; 61 and 66; 61 and 67; 61 and 68; 61 and 69; 62 and 63; 62 and 64; 62 and 65; 62 and 66; 62 and 67; 62 and 68; 62 and 69; 63 and 64; 63 and 65; 63 and 66; 63 and 67; 63 and 68; 63 and 69; 64 and 65; 64 and 66; 64 and 67; 64 and 68; 64 and 69; 65 and 66; 65 and 67; 65 and 68; 65 and 69; 66 and 67; 66 and 68; 66 and 69; 67 and 68; 67 and 69; 68 and 69;
h. For exon 51 (with, e.g., SluCas9), SEQ ID NOs: 243 and 244; 243 and 245; 243 and 246; 243 and 247; 243 and 248; 243 and 249; 243 and 250; 243 and 251; 243 and 252; 243 and 253; 243 and 254; 243 and 255; 243 and 256; 243 and 257; 243 and 258; 243 and 259; 243 and 260; 243 and 261; 243 and 262; 243 and 263; 243 and 264; 243 and 265; 243 and 266; 243 and 267; 243 and 268; 243 and 269; 244 and 245; 244 and 246; 244 and 247; 244 and 248; 244 and 249; 244 and 250; 244 and 251; 244 and 252; 244 and 253; 244 and 254; 244 and 255; 244 and 256; 244 and 257; 244 and 258; 244 and 259; 244 and 260; 244 and 261; 244 and 262; 244 and 263; 244 and 264; 244 and 265; 244 and 266; 244 and 267; 244 and 268; 244 and 269; 245 and 246; 245 and 247; 245 and 248; 245 and 249; 245 and 250; 245 and 251; 245 and 252; 245 and 253; 245 and 254; 245 and 255; 245 and 256; 245 and 257; 245 and 258; 245 and 259; 245 and 260; 245 and 261; 245 and 262; 245 and 263; 245 and 264; 245 and 265; 245 and 266; 245 and 267; 245 and 268; 245 and 269; 246 and 247; 246 and 248; 246 and 249; 246 and 250; 246 and 251; 246 and 252; 246 and 253; 246 and 254; 246 and 255; 246 and 256; 246 and 257; 246 and 258; 246 and 259; 246 and 260; 246 and 261; 246 and 262; 246 and 263; 246 and 264; 246 and 265; 246 and 266; 246 and 267; 246 and 268; 246 and 269; 247 and 248; 247 and 249; 247 and 250; 247 and 251; 247 and 252; 247 and 253; 247 and 254; 247 and 255; 247 and 256; 247 and 257; 247 and 258; 247 and 259; 247 and 260; 247 and 261; 247 and 262; 247 and 263; 247 and 264; 247 and 265; 247 and 266; 247 and 267; 247 and 268; 247 and 269; 248 and 249; 248 and 250; 248 and 251; 248 and 252; 248 and 253; 248 and 254; 248 and 255; 248 and 256; 248 and 257; 248 and 258; 248 and 259; 248 and 260; 248 and 261; 248 and 262; 248 and 263; 248 and 264; 248 and 265; 248 and 266; 248 and 267; 248 and 268; 248 and 269; 249 and 250; 249 and 251; 249 and 252; 249 and 253; 249 and 254; 249 and 255; 249 and 256; 249 and 257; 249 and 258; 249 and 259; 249 and 260; 249 and 261; 249 and 262; 249 and 263; 249 and 264; 249 and 265; 249 and 266; 249 and 267; 249 and 268; 249 and 269; 250 and 251; 250 and 252; 250 and 253; 250 and 254; 250 and 255; 250 and 256; 250 and 257; 250 and 258; 250 and 259; 250 and 260; 250 and 261; 250 and 262; 250 and 263; 250 and 264; 250 and 265; 250 and 266; 250 and 267; 250 and 268; 250 and 269; 251 and 252; 251 and 253; 251 and 254; 251 and 255; 251 and 256; 251 and 257; 251 and 258; 251 and 259; 251 and 260; 251 and 261; 251 and 262; 251 and 263; 251 and 264; 251 and 265; 251 and 266; 251 and 267; 251 and 268; 251 and 269; 252 and 253; 252 and 254; 252 and 255; 252 and 256; 252 and 257; 252 and 258; 252 and 259; 252 and 260; 252 and 261; 252 and 262; 252 and 263; 252 and 264; 252 and 265; 252 and 266; 252 and 267; 252 and 268; 252 and 269; 253 and 254; 253 and 255; 253 and 256; 253 and 257; 253 and 258; 253 and 259; 253 and 260; 253 and 261; 253 and 262; 253 and 263; 253 and 264; 253 and 265; 253 and 266; 253 and 267; 253 and 268; 253 and 269; 254 and 255; 254 and 256; 254 and 257; 254 and 258; 254 and 259; 254 and 260; 254 and 261; 254 and 262; 254 and 263; 254 and 264; 254 and 265; 254 and 266; 254 and 267; 254 and 268; 254 and 269; 255 and 256; 255 and 257; 255 and 258; 255 and 259; 255 and 260; 255 and 261; 255 and 262; 255 and 263; 255 and 264; 255 and 265; 255 and 266; 255 and 267; 255 and 268; 255 and 269; 256 and 257; 256 and 258; 256 and 259; 256 and 260; 256 and 261; 256 and 262; 256 and 263; 256 and 264; 256 and 265; 256 and 266; 256 and 267; 256 and 268; 256 and 269; 257 and 258; 257 and 259; 257 and 260; 257 and 261; 257 and 262; 257 and 263; 257 and 264; 257 and 265; 257 and 266; 257 and 267; 257 and 268; 257 and 269; 258 and 259; 258 and 260; 258 and 261; 258 and 262; 258 and 263; 258 and 264; 258 and 265; 258 and 266; 258 and 267; 258 and 268; 258 and 269; 259 and 260; 259 and 261; 259 and 262; 259 and 263; 259 and 264; 259 and 265; 259 and 266; 259 and 267; 259 and 268; 259 and 269; 260 and 261; 260 and 262; 260 and 263; 260 and 264; 260 and 265; 260 and 266; 260 and 267; 260 and 268; 260 and 269; 261 and 262; 261 and 263; 261 and 264; 261 and 265; 261 and 266; 261 and 267; 261 and 268; 261 and 269; 262 and 263; 262 and 264; 262 and 265; 262 and 266; 262 and 267; 262 and 268; 262 and 269; 263 and 264; 263 and 265; 263 and 266; 263 and 267; 263 and 268; 263 and 269; 264 and 265; 264 and 266; 264 and 267; 264 and 268; 264 and 269; 265 and 266; 265 and 267; 265 and 268; 265 and 269; 266 and 267; 266 and 268; 266 and 269; 267 and 268; 267 and 269; 268 and 269;
i. For exon 53 (with, e.g., SaCas9), SEQ ID NOs: 16 and 17; 16 and 18; 16 and 19; 16 and 20; 16 and 21; 16 and 22; 16 and 23; 16 and 24; 16 and 25; 16 and 26; 17 and 18; 17 and 19; 17 and 20; 17 and 21; 17 and 22; 17 and 23; 17 and 24; 17 and 25; 17 and 26; 18 and 19; 18 and 20; 18 and 21; 18 and 22; 18 and 23; 18 and 24; 18 and 25; 18 and 26; 19 and 20; 19 and 21; 19 and 22; 19 and 23; 19 and 24; 19 and 25; 19 and 26; 20 and 21; 20 and 22; 20 and 23; 20 and 24; 20 and 25; 20 and 26; 21 and 22; 21 and 23; 21 and 24; 21 and 25; 21 and 26; 22 and 23; 22 and 24; 22 and 25; 22 and 26; 23 and 24; 23 and 25; 23 and 26; 24 and 25; 24 and 26; 25 and 26; 71 and 72; 71 and 73; 71 and 74; 71 and 75; 71 and 76; 71 and 77; 71 and 78; 71 and 84; 71 and 85; 71 and 86; 71 and 87; 71 and 88; 71 and 89; 71 and 90; 71 and 91; 71 and 92; 71 and 93; 71 and 94; 71 and 95; 71 and 96; 71 and 97; 71 and 98; 71 and 99; 71 and 100; 71 and 101; 71 and 102; 72 and 73; 72 and 74; 72 and 75; 72 and 76; 72 and 77; 72 and 78; 72 and 84; 72 and 85; 72 and 86; 72 and 87; 72 and 88; 72 and 89; 72 and 90; 72 and 91; 72 and 92; 72 and 93; 72 and 94; 72 and 95; 72 and 96; 72 and 97; 72 and 98; 72 and 99; 72 and 100; 72 and 101; 72 and 102; 73 and 74; 73 and 75; 73 and 76; 73 and 77; 73 and 78; 73 and 84; 73 and 85; 73 and 86; 73 and 87; 73 and 88; 73 and 89; 73 and 90; 73 and 91; 73 and 92; 73 and 93; 73 and 94; 73 and 95; 73 and 96; 73 and 97; 73 and 98; 73 and 99; 73 and 100; 73 and 101; 73 and 102; 74 and 75; 74 and 76; 74 and 77; 74 and 78; 74 and 84; 74 and 85; 74 and 86; 74 and 87; 74 and 88; 74 and 89; 74 and 90; 74 and 91; 74 and 92; 74 and 93; 74 and 94; 74 and 95; 74 and 96; 74 and 97; 74 and 98; 74 and 99; 74 and 100; 74 and 101; 74 and 102; 75 and 76; 75 and 77; 75 and 78; 75 and 84; 75 and 85; 75 and 86; 75 and 87; 75 and 88; 75 and 89; 75 and 90; 75 and 91; 75 and 92; 75 and 93; 75 and 94; 75 and 95; 75 and 96; 75 and 97; 75 and 98; 75 and 99; 75 and 100; 75 and 101; 75 and 102; 76 and 77; 76 and 78; 76 and 84; 76 and 85; 76 and 86; 76 and 87; 76 and 88; 76 and 89; 76 and 90; 76 and 91; 76 and 92; 76 and 93; 76 and 94; 76 and 95; 76 and 96; 76 and 97; 76 and 98; 76 and 99; 76 and 100; 76 and 101; 76 and 102; 77 and 78; 77 and 84; 77 and 85; 77 and 86; 77 and 87; 77 and 88; 77 and 89; 77 and 90; 77 and 91; 77 and 92; 77 and 93; 77 and 94; 77 and 95; 77 and 96; 77 and 97; 77 and 98; 77 and 99; 77 and 100; 77 and 101; 77 and 102; 78 and 84; 78 and 85; 78 and 86; 78 and 87; 78 and 88; 78 and 89; 78 and 90; 78 and 91; 78 and 92; 78 and 93; 78 and 94; 78 and 95; 78 and 96; 78 and 97; 78 and 98; 78 and 99; 78 and 100; 78 and 101; 78 and 102; 84 and 85; 84 and 86; 84 and 87; 84 and 88; 84 and 89; 84 and 90; 84 and 91; 84 and 92; 84 and 93; 84 and 94; 84 and 95; 84 and 96; 84 and 97; 84 and 98; 84 and 99; 84 and 100; 84 and 101; 84 and 102; 85 and 86; 85 and 87; 85 and 88; 85 and 89; 85 and 90; 85 and 91; 85 and 92; 85 and 93; 85 and 94; 85 and 95; 85 and 96; 85 and 97; 85 and 98; 85 and 99; 85 and 100; 85 and 101; 85 and 102; 86 and 87; 86 and 88; 86 and 89; 86 and 90; 86 and 91; 86 and 92; 86 and 93; 86 and 94; 86 and 95; 86 and 96; 86 and 97; 86 and 98; 86 and 99; 86 and 100; 86 and 101; 86 and 102; 87 and 88; 87 and 89; 87 and 90; 87 and 91; 87 and 92; 87 and 93; 87 and 94; 87 and 95; 87 and 96; 87 and 97; 87 and 98; 87 and 99; 87 and 100; 87 and 101; 87 and 102; 88 and 89; 88 and 90; 88 and 91; 88 and 92; 88 and 93; 88 and 94; 88 and 95; 88 and 96; 88 and 97; 88 and 98; 88 and 99; 88 and 100; 88 and 101; 88 and 102; 89 and 90; 89 and 91; 89 and 92; 89 and 93; 89 and 94; 89 and 95; 89 and 96; 89 and 97; 89 and 98; 89 and 99; 89 and 100; 89 and 101; 89 and 102; 90 and 91; 90 and 92; 90 and 93; 90 and 94; 90 and 95; 90 and 96; 90 and 97; 90 and 98; 90 and 99; 90 and 100; 90 and 101; 90 and 102; 91 and 92; 91 and 93; 91 and 94; 91 and 95; 91 and 96; 91 and 97; 91 and 98; 91 and 99; 91 and 100; 91 and 101; 91 and 102; 92 and 93; 92 and 94; 92 and 95; 92 and 96; 92 and 97; 92 and 98; 92 and 99; 92 and 100; 92 and 101; 92 and 102; 93 and 94; 93 and 95; 93 and 96; 93 and 97; 93 and 98; 93 and 99; 93 and 100; 93 and 101; 93 and 102; 94 and 95; 94 and 96; 94 and 97; 94 and 98; 94 and 99; 94 and 100; 94 and 101; 94 and 102; 95 and 96; 95 and 97; 95 and 98; 95 and 99; 95 and 100; 95 and 101; 95 and 102; 96 and 97; 96 and 98; 96 and 99; 96 and 100; 96 and 101; 96 and 102; 97 and 98; 97 and 99; 97 and 100; 97 and 101; 97 and 102; 98 and 99; 98 and 100; 98 and 101; 98 and 102; 99 and 100; 99 and 101; 99 and 102; 100 and 101; 100 and 102; 101 and 102;
j. For exon 53 (with, e.g., SluCas9), SEQ ID NOs: 270 and 271; 270 and 272; 270 and 273; 270 and 274; 270 and 275; 270 and 276; 270 and 277; 270 and 278; 270 and 279; 270 and 280; 270 and 281; 270 and 282; 270 and 283; 270 and 284; 270 and 285; 270 and 286; 270 and 287; 270 and 288; 270 and 289; 270 and 290; 270 and 291; 270 and 292; 271 and 272; 271 and 273; 271 and 274; 271 and 275; 271 and 276; 271 and 277; 271 and 278; 271 and 279; 271 and 280; 271 and 281; 271 and 282; 271 and 283; 271 and 284; 271 and 285; 271 and 286; 271 and 287; 271 and 288; 271 and 289; 271 and 290; 271 and 291; 271 and 292; 272 and 273; 272 and 274; 272 and 275; 272 and 276; 272 and 277; 272 and 278; 272 and 279; 272 and 280; 272 and 281; 272 and 282; 272 and 283; 272 and 284; 272 and 285; 272 and 286; 272 and 287; 272 and 288; 272 and 289; 272 and 290; 272 and 291; 272 and 292; 273 and 274; 273 and 275; 273 and 276; 273 and 277; 273 and 278; 273 and 279; 273 and 280; 273 and 281; 273 and 282; 273 and 283; 273 and 284; 273 and 285; 273 and 286; 273 and 287; 273 and 288; 273 and 289; 273 and 290; 273 and 291; 273 and 292; 274 and 275; 274 and 276; 274 and 277; 274 and 278; 274 and 279; 274 and 280; 274 and 281; 274 and 282; 274 and 283; 274 and 284; 274 and 285; 274 and 286; 274 and 287; 274 and 288; 274 and 289; 274 and 290; 274 and 291; 274 and 292; 275 and 276; 275 and 277; 275 and 278; 275 and 279; 275 and 280; 275 and 281; 275 and 282; 275 and 283; 275 and 284; 275 and 285; 275 and 286; 275 and 287; 275 and 288; 275 and 289; 275 and 290; 275 and 291; 275 and 292; 276 and 277; 276 and 278; 276 and 279; 276 and 280; 276 and 281; 276 and 282; 276 and 283; 276 and 284; 276 and 285; 276 and 286; 276 and 287; 276 and 288; 276 and 289; 276 and 290; 276 and 291; 276 and 292; 277 and 278; 277 and 279; 277 and 280; 277 and 281; 277 and 282; 277 and 283; 277 and 284; 277 and 285; 277 and 286; 277 and 287; 277 and 288; 277 and 289; 277 and 290; 277 and 291; 277 and 292; 278 and 279; 278 and 280; 278 and 281; 278 and 282; 278 and 283; 278 and 284; 278 and 285; 278 and 286; 278 and 287; 278 and 288; 278 and 289; 278 and 290; 278 and 291; 278 and 292; 279 and 280; 279 and 281; 279 and 282; 279 and 283; 279 and 284; 279 and 285; 279 and 286; 279 and 287; 279 and 288; 279 and 289; 279 and 290; 279 and 291; 279 and 292; 280 and 281; 280 and 282; 280 and 283; 280 and 284; 280 and 285; 280 and 286; 280 and 287; 280 and 288; 280 and 289; 280 and 290; 280 and 291; 280 and 292; 281 and 282; 281 and 283; 281 and 284; 281 and 285; 281 and 286; 281 and 287; 281 and 288; 281 and 289; 281 and 290; 281 and 291; 281 and 292; 282 and 283; 282 and 284; 282 and 285; 282 and 286; 282 and 287; 282 and 288; 282 and 289; 282 and 290; 282 and 291; 282 and 292; 283 and 284; 283 and 285; 283 and 286; 283 and 287; 283 and 288; 283 and 289; 283 and 290; 283 and 291; 283 and 292; 284 and 285; 284 and 286; 284 and 287; 284 and 288; 284 and 289; 284 and 290; 284 and 291; 284 and 292; 285 and 286; 285 and 287; 285 and 288; 285 and 289; 285 and 290; 285 and 291; 285 and 292; 286 and 287; 286 and 288; 286 and 289; 286 and 290; 286 and 291; 286 and 292; 287 and 288; 287 and 289; 287 and 290; 287 and 291; 287 and 292; 288 and 289; 288 and 290; 288 and 291; 288 and 292; 289 and 290; 289 and 291; 289 and 292; 290 and 291; 290 and 292; 291 and 292; 924 and 270; 924 and 272; 924 and 273; 924 and 274; 924 and 275; 924 and 276; 924 and 277; 924 and 278; 924 and 279; 924 and 280; 924 and 281; 924 and 282; 924 and 283; 924 and 284; 924 and 285; 924 and 286; 924 and 287; 924 and 288; 924 and 289; 924 and 290; 924 and 291; 924 and 292; 925 and 270; 925 and 272; 925 and 273; 925 and 274; 925 and 275; 925 and 276; 925 and 277; 925 and 278; 925 and 279; 925 and 280; 925 and 281; 925 and 282; 925 and 283; 925 and 284; 925 and 285; 925 and 286; 925 and 287; 925 and 288; 925 and 289; 925 and 290; 925 and 291; 925 and 292; 926 and 270; 926 and 272; 926 and 273; 926 and 274; 926 and 275; 926 and 276; 926 and 277; 926 and 278; 926 and 279; 926 and 280; 926 and 281; 926 and 282; 926 and 283; 926 and 284; 926 and 285; 926 and 286; 926 and 287; 926 and 288; 926 and 289; 926 and 290; 926 and 291; 926 and 292; 927 and 270; 927 and 272; 927 and 273; 927 and 274; 927 and 275; 927 and 276; 927 and 277; 927 and 278; 927 and 279; 927 and 280; 927 and 281; 927 and 282; 927 and 283; 927 and 284; 927 and 285; 927 and 286; 927 and 287; 927 and 288; 927 and 289; 927 and 290; 927 and 291; 927 and 292; 928 and 270; 928 and 272; 928 and 273; 928 and 274; 928 and 275; 928 and 276; 928 and 277; 928 and 278; 928 and 279; 928 and 280; 928 and 281; 928 and 282; 928 and 283; 928 and 284; 928 and 285; 928 and 286; 928 and 287; 928 and 288; 928 and 289; 928 and 290; 928 and 291; 928 and 292; 929 and 270; 929 and 272; 929 and 273; 929 and 274; 929 and 275; 929 and 276; 929 and 277; 929 and 278; 929 and 279; 929 and 280; 929 and 281; 929 and 282; 929 and 283; 929 and 284; 929 and 285; 929 and 286; 929 and 287; 929 and 288; 929 and 289; 929 and 290; 929 and 291; 929 and 292; 930 and 270; 930 and 272; 930 and 273; 930 and 274; 930 and 275; 930 and 276; 930 and 277; 930 and 278; 930 and 279; 930 and 280; 930 and 281; 930 and 282; 930 and 283; 930 and 284; 930 and 285; 930 and 286; 930 and 287; 930 and 288; 930 and 289; 930 and 290; 930 and 291; 930 and 292; 931 and 270; 931 and 272; 931 and 273; 931 and 274; 931 and 275; 931 and 276; 931 and 277; 931 and 278; 931 and 279; 931 and 280; 931 and 281; 931 and 282; 931 and 283; 931 and 284; 931 and 285; 931 and 286; 931 and 287; 931 and 288; 931 and 289; 931 and 290; 931 and 291; 931 and 292; 932 and 270; 932 and 272; 932 and 273; 932 and 274; 932 and 275; 932 and 276; 932 and 277; 932 and 278; 932 and 279; 932 and 280; 932 and 281; 932 and 282; 932 and 283; 932 and 284; 932 and 285; 932 and 286; 932 and 287; 932 and 288; 932 and 289; 932 and 290; 932 and 291; 932 and 292; 933 and 270; 933 and 272; 933 and 273; 933 and 274; 933 and 275; 933 and 276; 933 and 277; 933 and 278; 933 and 279; 933 and 280; 933 and 281; 933 and 282; 933 and 283; 933 and 284; 933 and 285; 933 and 286; 933 and 287; 933 and 288; 933 and 289; 933 and 290; 933 and 291; 933 and 292; 934 and 270; 934 and 272; 934 and 273; 934 and 274; 934 and 275; 934 and 276; 934 and 277; 934 and 278; 934 and 279; 934 and 280; 934 and 281; 934 and 282; 934 and 283; 934 and 284; 934 and 285; 934 and 286; 934 and 287; 934 and 288; 934 and 289; 934 and 290; 934 and 291; 934 and 292; 935 and 270; 935 and 272; 935 and 273; 935 and 274; 935 and 275; 935 and 276; 935 and 277; 935 and 278; 935 and 279; 935 and 280; 935 and 281; 935 and 282; 935 and 283; 935 and 284; 935 and 285; 935 and 286; 935 and 287; 935 and 288; 935 and 289; 935 and 290; 935 and 291; 935 and 292; 936 and 270; 936 and 272; 936 and 273; 936 and 274; 936 and 275; 936 and 276; 936 and 277; 936 and 278; 936 and 279; 936 and 280; 936 and 281; 936 and 282; 936 and 283; 936 and 284; 936 and 285; 936 and 286; 936 and 287; 936 and 288; 936 and 289; 936 and 290; 936 and 291; 936 and 292; 937 and 270; 937 and 272; 937 and 273; 937 and 274; 937 and 275; 937 and 276; 937 and 277; 937 and 278; 937 and 279; 937 and 280; 937 and 281; 937 and 282; 937 and 283; 937 and 284; 937 and 285; 937 and 286; 937 and 287; 937 and 288; 937 and 289; 937 and 290; 937 and 291; 937 and 292; 938 and 270; 938 and 272; 938 and 273; 938 and 274; 938 and 275; 938 and 276; 938 and 277; 938 and 278; 938 and 279; 938 and 280; 938 and 281; 938 and 282; 938 and 283; 938 and 284; 938 and 285; 938 and 286; 938 and 287; 938 and 288; 938 and 289; 938 and 290; 938 and 291; 938 and 292; 950 and 275; 951 and 275; 952 and 275; 953 and 275; 954 and 275; or 955 and 275;
k. For exon 44 with SluCas9: SEQ ID NOs: 200 and 203; 200 and 204; 202 and 203; 202 and 205; 202 and 206; 202 and 207; 204 and 208; 204 and 205; 204 and 206; 204 and 207; or 204 and 208;
l. For exon 45 with SluCas9, SEQ ID NOs: 223 and 230; or 224 and 212.
m. For exon 50 with SluCas9, SEQ ID NOs: 231 and 232; 231 and 234; 231 and 236; 231 and 237; 236 and 233; 236 and 235; 236 and 238; 236 and 240; or 236 and 241;
n. For exon 51 with SaCas9, SEQ ID NOs: 28 and 57; 31 and 57; 31 and 46; 32 and 69; 33 and 57; 33 and 69; 34 and 57; 36 and 69; 37 and 69; 37 and 46; 39 and 46; 40 and 46; 46 and 35; 46 and 62; 46 and 65; 46 and 67; 47 and 57; 48 and 57; 49 and 57; 51 and 57; 53 and 46; 54 and 46; 55 and 46; 58 and 46; 59 and 46; 60 and 46; 61 and 69; or 69 and 30;
o. For exon 51 with SluCas9, SEQ ID NOs: 243 and 252; 245 and 252; 252 and 253; 252 and 254; 252 and 256; 252 and 257; 252 and 258; 252 and 262; 252 and 264; 252 and 265; or 252 and 266;
p. For exon 53 with SaCas9, SEQ ID NOs: 86 and 96; 87 and 96; 88 and 97; 89 and 96; 90 and 97; 92 and 77; 92 and 78; 92 and 96; 92 and 99; 93 and 98; 93 and 102; 94 and 100; 95 and 77; 95 and 78; 95 and 99; 96 and 100; 97 and 98; 97 and 102; 98 and 73; or 102 and 73; and
q. For exon 53 with SluCas9, SEQ ID NOs: 278 and 290; 278 and 292; 281 and 291; 283 and 290; 283 and 292; 287 and 291; 272 and 290; 290 and 291; or 272 and 292.

In some embodiments, a method for treating DMD is provided comprising administering a composition comprising a Cas protein or a nucleic acid encoding a Cas protein and a pair of guide RNAs, wherein the pair of guide RNAs comprise or consist of any one of the pairs of guide sequences of any one of Tables 1B or 1D for exon 44, Tables 2B or 2D for exon 45, Tables 3B or 3D for exon 50, Tables 4B or 4D for exon 51, and Tables 5B or 5D for exon 53.

In some embodiments, a method for treating DMD is provided comprising administering a Cas protein or a nucleic acid encoding a Cas protein and a composition comprising one or more nucleic acid molecules encoding a pair of guide RNAs, wherein the pair of guide RNAs comprise or consist of one or more nucleic acid molecules encoding any one of the pairs of guide sequences of any one of Tables 1B or 1D for exon 44, Tables 2B or 2D for exon 45, Tables 3B or 3D for exon 50, Tables 4B or 4D for exon 51, and Tables 5B or 5D for exon 53.

In some embodiments, a method for treating DMD is provided comprising administering a Cas protein or a nucleic acid encoding a Cas protein and a composition comprising a pair of guide RNAs comprising or consisting of any one of the pairs of guide sequences of any one of the following:
a. For exon 44 with SluCas9, SEQ ID NOs: 200 and 203; 200 and 204; 202 and 203; 202 and 205; 202 and 206; 202 and 207; 204 and 208; 204 and 205; 204 and 206; 204 and 207; or 204 and 208;
b. For exon 45 with SluCas9, SEQ ID NOs: 223 and 230; or 224 and 212;
c. For exon 50 with SluCas9, SEQ ID NOs: 231 and 232; 231 and 234; 231 and 236; 231 and 237; 236 and 233; 236 and 235; 236 and 238; 236 and 240; or 236 and 241;
d. For exon 51 with SaCas9, SEQ ID NOs: 28 and 57; 31 and 57; 31 and 46; 32 and 69; 33 and 57; 33 and 69; 34 and 57; 36 and 69; 37 and 69; 37 and 46; 39 and 46; 40 and 46; 46 and 35; 46 and 62; 46 and 65; 46 and 67; 47 and 57; 48 and 57; 49 and 57; 51 and 57; 53 and 46; 54 and 46; 55 and 46; 58 and 46; 59 and 46; 60 and 46; 61 and 69; or 69 and 30;
e. For exon 51 with SluCas9, SEQ ID NOs: 243 and 252; 245 and 252; 252 and 253; 252 and 254; 252 and 256; 252 and 257; 252 and 258; 252 and 262; 252 and 264; 252 and 265; or 252 and 266;
f. For exon 53 with SaCas9, SEQ ID NOs: 86 and 96; 87 and 96; 88 and 97; 89 and 96; 90 and 97; 92 and 77; 92 and 78; 92 and 96; 92 and 99; 93 and 98; 93 and 102; 94 and 100; 95 and 77; 95 and 78; 95 and 99; 96 and 100; 97 and 98; 97 and 102; 98 and 73; or 102 and 73; and
g. For exon 53 with SluCas9, SEQ ID NOs: 278 and 290; 278 and 292; 281 and 291; 283 and 290; 283 and 292; 287 and 291; 272 and 290; 290 and 291; or 272 and 292.

In some embodiments, a method for treating DMD is provided comprising administering a Cas protein or a nucleic acid encoding a Cas protein and a composition comprising one or more nucleic acid molecules encoding a pair of guide RNAs, wherein the pair of guide RNAs comprise or consist of one or more nucleic acid molecules encoding any one of the pairs of guide sequences of any one of the following:
a. For exon 44 with SluCas9, SEQ ID NOs: 200 and 203; 200 and 204; 202 and 203; 202 and 205; 202 and 206; 202 and 207; 204 and 208; 204 and 205; 204 and 206; 204 and 207; or 204 and 208;
b. For exon 45 with SluCas9, SEQ ID NOs: 223 and 230; or 224 and 212.
c. For exon 50 with SluCas9, SEQ ID NOs: 231 and 232; 231 and 234; 231 and 236; 231 and 237; 236 and 233; 236 and 235; 236 and 238; 236 and 240; or 236 and 241;
d. For exon 51 with SaCas9, SEQ ID NOs: 28 and 57; 31 and 57; 31 and 46; 32 and 69; 33 and 57; 33 and 69; 34 and 57; 36 and 69; 37 and 69; 37 and 46; 39 and 46; 40 and 46; 46 and 35; 46 and 62; 46 and 65; 46 and 67; 47 and 57; 48 and 57; 49 and 57; 51 and 57; 53 and 46; 54 and 46; 55 and 46; 58 and 46; 59 and 46; 60 and 46; 61 and 69; or 69 and 30;
e. For exon 51 with SluCas9, SEQ ID NOs: 243 and 252; 245 and 252; 252 and 253; 252 and 254; 252 and 256; 252 and 257; 252 and 258; 252 and 262; 252 and 264; 252 and 265; or 252 and 266;
f. For exon 53 with SaCas9, SEQ ID NOs: 86 and 96; 87 and 96; 88 and 97; 89 and 96; 90 and 97; 92 and 77; 92 and 78; 92 and 96; 92 and 99; 93 and 98; 93 and 102; 94 and 100; 95 and 77; 95 and 78; 95 and 99; 96 and 100; 97 and 98; 97 and 102; 98 and 73; or 102 and 73; and
g. For exon 53 with SluCas9, SEQ ID NOs: 278 and 290; 278 and 292; 281 and 291; 283 and 290; 283 and 292; 287 and 291; 272 and 290; 290 and 291; or 272 and 292.

In some embodiments, a method for treating DMD further comprises administering a nucleic acid encoding an endonuclease. The appropriate endonuclease for use with each of the guide RNAs is provided herein, for example, in Table 6, column “enzyme.”

In some embodiments, the subject is a mammal. In some embodiments, the subject is human.

For treatment of a subject (e.g., a human), any of the compositions disclosed herein may be administered in a manner compatible with the dosage formulation and in such amount as is therapeutically effective. The compositions may be readily administered in a variety of dosage forms, such as injectable solutions. For parenteral administration in an aqueous solution, for example, the solution will generally be suitably buffered and the liquid diluent first rendered isotonic with, for example, sufficient saline or glucose. Such aqueous solutions may be used, for example, for intravenous, intramuscular, subcutaneous, and/or intraperitoneal administration.

### Combination Therapy

In some embodiments, the invention comprises combination therapies comprising any of the methods or uses described herein together with an additional therapy suitable for ameliorating DMD.

### Delivery of Guide RNA Compositions

The methods and uses disclosed herein may use any suitable approach for delivering the guide RNAs and compositions described herein. Exemplary delivery approaches include vectors, such as viral vectors; lipid nanoparticles; transfection; and electroporation. In some embodiments, vectors or LNPs associated with the single-vector guide RNAs/Cas9's disclosed herein are for use in preparing a medicament for treating DMD.

Lipid nanoparticles (LNPs) are a known means for delivery of nucleotide and protein cargo, and may be used for delivery of the guide RNAs, compositions, or pharmaceutical formulations disclosed herein. In some embodiments, the LNPs deliver nucleic acid, protein, or nucleic acid together with protein.

Electroporation is a well-known means for delivery of cargo, and any electroporation methodology may be used for delivering the single vectors disclosed herein.

In some embodiments, the invention comprises a method for delivering any one of the single vectors disclosed herein to an ex vivo cell, wherein the guide RNA is encoded by a vector, associated with an LNP, or in aqueous solution. In some embodiments, the guide RNA/LNP or guide RNA is also associated with a Cas9 or sequence encoding Cas9 (e.g., in the same vector, LNP, or solution).

In some embodiments, the disclosure provides for methods of using any of the guides, endonucleases, cells, or compositions disclosed herein in research methods. For example, any of the guides or endonucleases disclosed herein may be used alone or in combination in experiments under various parameters (e.g., temperatures, pH, types of cells) or combined with other reagents to evaluate the activity of the guides and/or endonucleases.

### EXAMPLES

The following examples are provided to illustrate certain disclosed embodiments and are not to be construed as limiting the scope of this disclosure in any way.

### Example 1: Exemplary DMD sgRNAs

Guide RNA comprising the guide sequences shown in Table 6 were prepared according to standard methods in a single guide (sgRNA) format. A single AAV vector, or two AAV vectors, were prepared that expresses one or more of the guide RNAs and a SaCas9 (for guide sequences having SEQ ID NOs: 1-159) or SluCas9 (for guide sequences having SEQ ID NOs: 200-292, 924-938, or 950-955). The AAV vector was administered to cells in vitro to assess the ability of the AAV to express the guide RNA and Cas9, edit the targeted exon (see Table 6), and thereby treat DMD.

### Example 2: Evaluation of sgRNA Pairs

### A. Materials and Methods

1. sgRNA Selection

A subset of SaCas9-KKH or SluCas9 sgRNAs found within the DMD gene was selected for indel frequency and profile evaluation. The selected sgRNAs for pair evaluation are shown in Table 6 and were prepared according to standard methods. The criteria used to select these sgRNAs included their potential to induce exon refraining and or skipping as a pair, in addition to the existence of a mouse, dog and NHP homologue counterpart. This selection included 4 sgRNAs located within exon 45, 43 sgRNAs located within exon 51 and 29 sgRNAs located within exon 53. The number of predicted off target sites was determined for each sgRNA.

### 2. Human Primary Skeletal Muscle Myoblasts (HsMMs) Culture

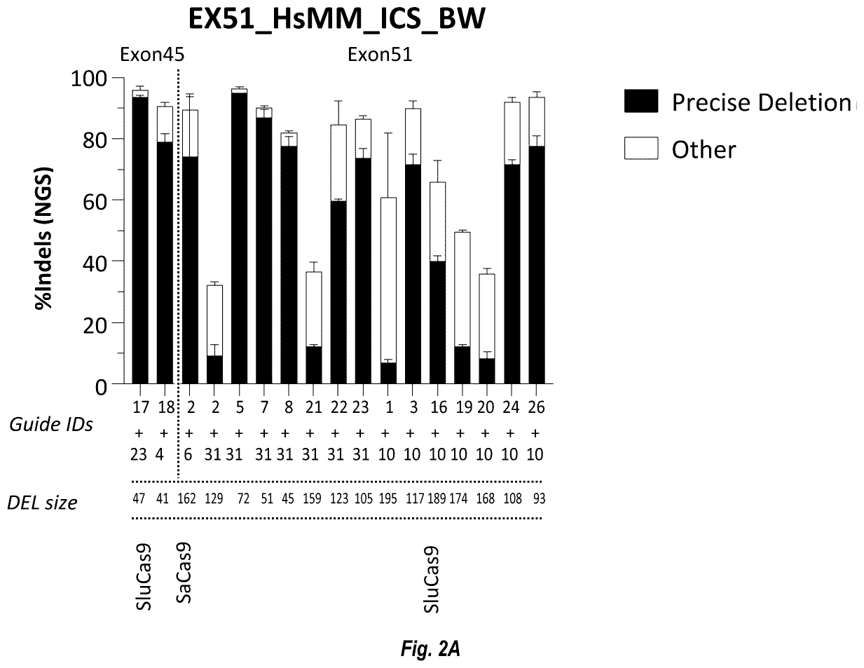

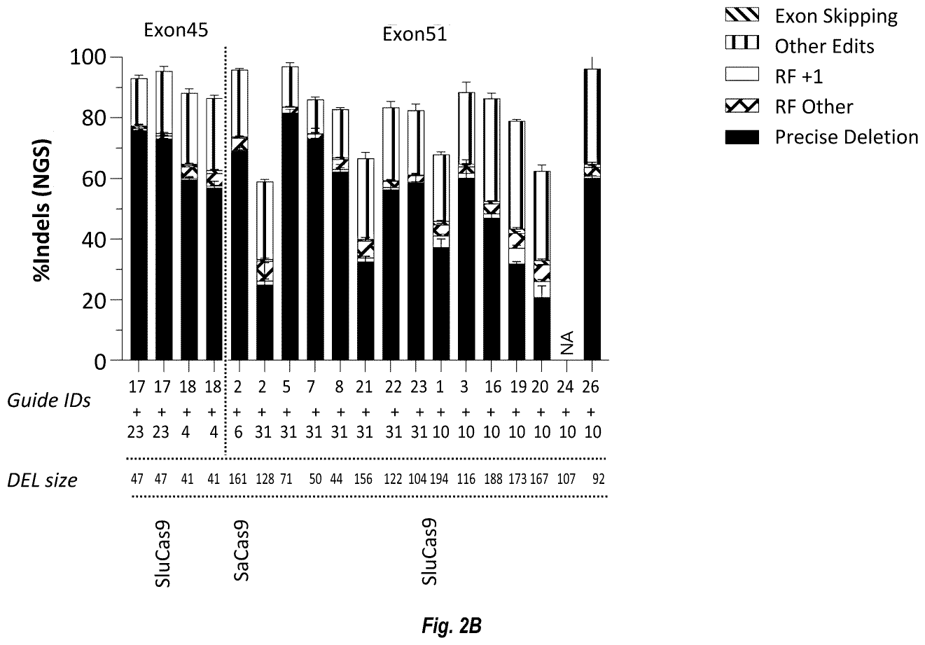

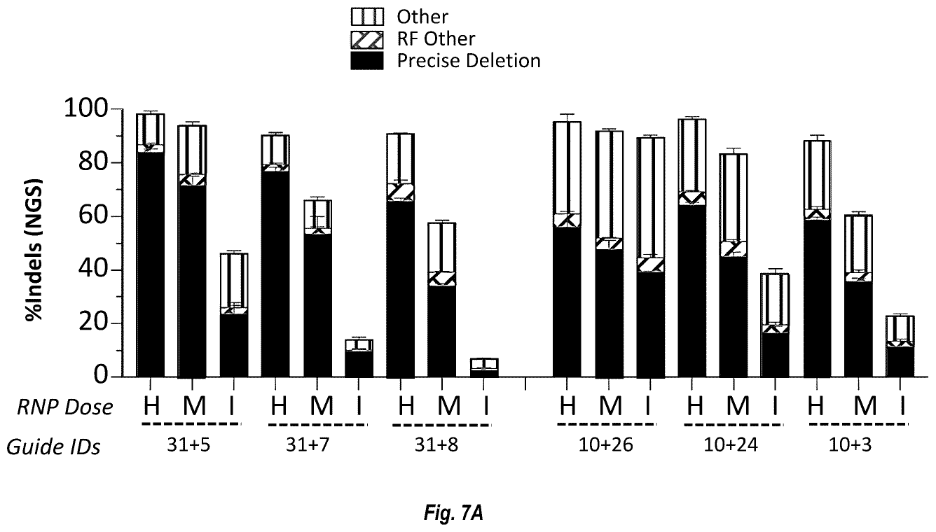

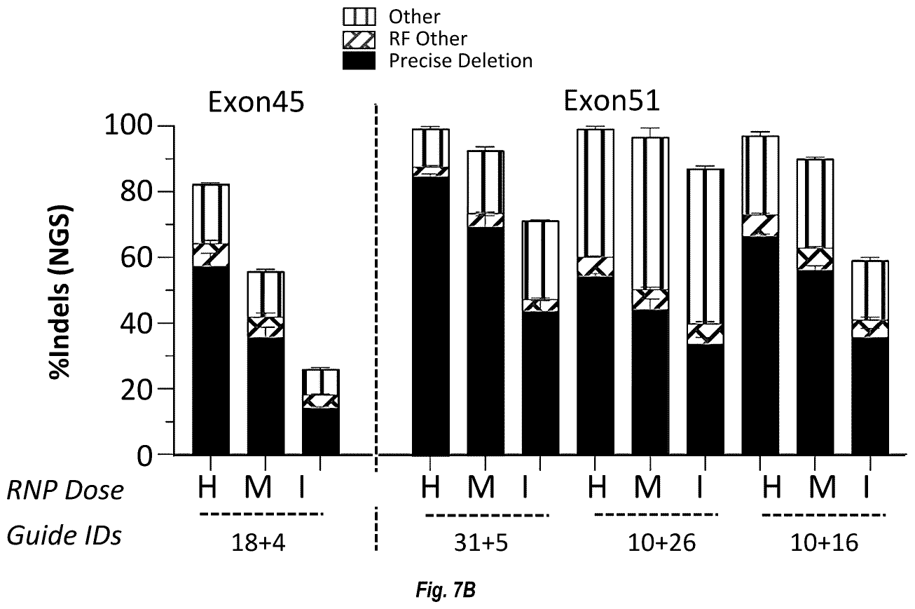

On day one, frozen vials of HsMMs (lot 20TL070666 for FIGS. 2, 6, and 7A; lot 18TL269121 for FIG. 7B) were thawed and grown in complete growth media in a 37° C., 5% CO₂, humidified incubator in T75 flasks. On day two, the growth media was renewed. On day three, the cells were passaged into T150 flasks. On day five, the media in each flask were changed. On day 6, the cells were harvested and electroporated with RNP (nucleofection).

### 3. Nucleofection of HsMM Cells

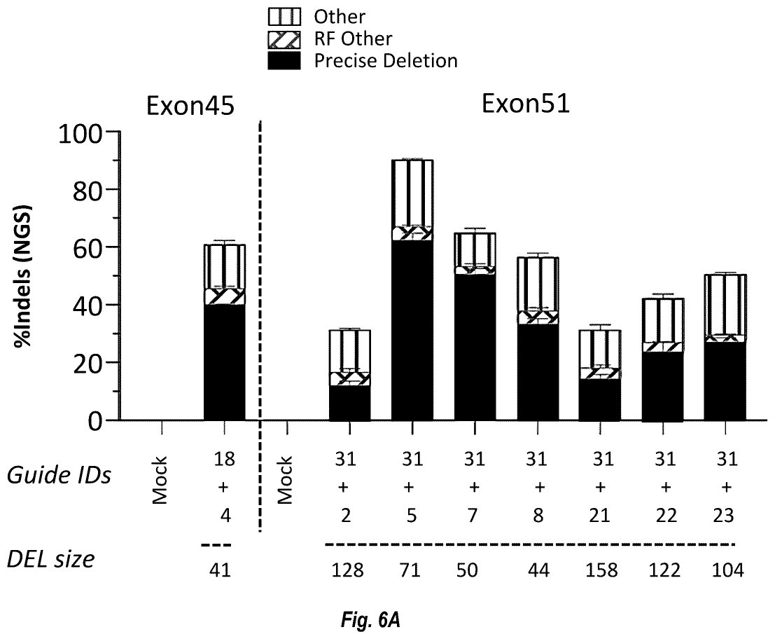

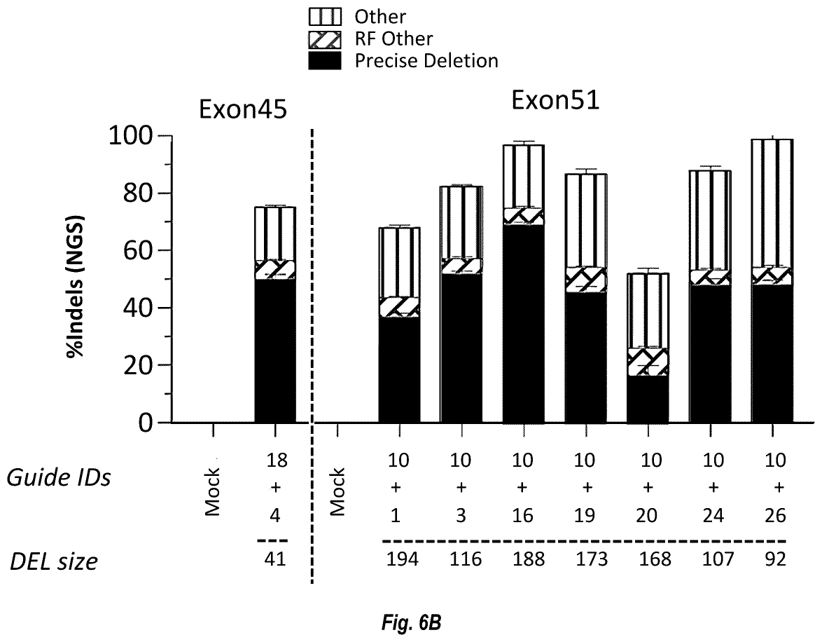

The selected sgRNAs for pair evaluation are shown in Tables 1A-1D and 2 and were chemically synthesized (Sythego). The Cas9 nuclease is recombinant purified SluCas9 (Aldevron). HsMM cells were nucleofected with RNP using the Lonza 4D nucleofector. For each sample, formulated RNP and 0.3×10⁶ cells were mixed in P5 solution. For dual-cut samples that contain two gRNAs, two separate RNPs were formulated with either gRNA of the pair. To formulate RNPs, the gRNA and protein were incubated for about 20 minutes at room temperature. For the experiment shown in FIG. 2, the RNPs were prepared in a 3:1 gRNA:Cas9 ratio at 18.75 pmols of sgRNA and of 6.25 pmols of Cas9; total of 37.5 pmols of RNA component and 12.5 pmols of Cas9 component. For the experiment shown in FIG. 6, the RNPs were prepared in a 6:1 gRNA:Cas9 ratio at 18.75 pmols of sgRNA and of 3.125 pmols of Cas9; total of 37.5 pmols of RNA component and 6.25 pmols of Cas9 component. For the experiment shown in FIG. 7, the RNPs were prepared in a 6:1 gRNA:Cas9 ratio at three different total RNP doses: i) high dose (H) at 75 pmol of sgRNA with 12.5 pmols of Cas9, ii) medium dose (M) at 37.5 pmol of sgRNA with 6.25 pmol of Cas9, and iii) low dose (L) 18.75 pmols of sgRNA and of 3.125 pmols of Cas9. For dual RNP nucleofections (FIG. 7), the total RNP dose reflects the sum of each RNP, e.g., high dose includes 37.5 pmols of sgRNA-1, 6.25 pmol Cas9 as RNP1, 37.5 pmols of sgRNA-2, and 6.25 pmol Cas9 as RNP2. After electroporation, 80 μL of complete growth media was added to each sample and samples were incubated in a 37° C., 5% CO², humidified incubator for 10 minutes. After this recovery, the samples were transferred to 12-well plates containing 2 mL of complete growth media that had been previously equilibrated in a 37° C., 5% CO², humidified incubator.

4. Cell Harvesting and gDNA Extraction

To determine cell viability 48 hours after nucleofection, the cells were stained with Hoechst and Propidium Iodide (Life Technologies). Cell viability was then assessed using ImageXpress Micro (Molecular Devices). In general, samples with an overall cell viability above 80% were harvested and analyzed for indel analysis. To isolate genomic DNA from HsMMs, the cells were washed with saline buffer, trypsinized and centrifuged. The cell pellets were treated with lysis buffer from the Maxwell RSC Blood DNA Kit (Promega #AS1400), and genomic DNAs were extracted using a Maxwell® RSC48 instrument (Promega #AS8500) according to the manufacturer's instruction. The concentrations of genomic DNAs were determined using Qubit™ 1×dsDNA HS Assay Kit (Thermo Fisher Scientific Q33231) according to the manufacturer's instruction.

### 5. Sequencing Analysis

For the data in FIGS. 6-8 and 10, the editing outcomes from the sequencing data were characterized computationally. Indel frequencies at the cut site were tabulated using the reference amplicon sequence, protospacer sequence, cleavage offset, and the size of the quantification window centered around the cut site.

A stringent set of QC criteria was applied to filter poor quality samples. These criteria included:
Average base quality in the sample
Minimum fraction of reads that are of high sequencing quality
Minimum fraction of reads with a minimum read length post-trimming
Minimum fraction of reads remaining after decontamination (i.e., removing phiX reads)
Minimum fraction of reads that successfully merged
Minimum fraction of reads that successfully aligned
Minimum number of aligned reads

For samples passing all QC criteria, indels identified were classified into three expected types of indels:
Precise dual-cut: Edits that properly reframe the transcript resulting from the expected cuts from both gRNAs.
Refraining edits: Indels other than precise dual-cut that properly reframe the transcript.
Other edits: Indels that do not properly reframe the transcript.
   6. For Each Sample, Total % Editing and % Editing for Each Type of Expected Indels (Precise Dual-Cut, Reframing Edits, Other Edits) were Reported. Transfection of HEK293FT Cells

To evaluate indel frequency and profile, human HEK293FT cell line was used. HEK293FT cells were transfected in 12-well plates with 750 ng plasmid+2.5 μL of Lipofectamine 2000. Three days after transfection, cells were trypsinized and sorted for green fluorescent protein (GFP). GFP-positive cells were sorted directly into lysis buffer, and DNA extraction was performed for exon 51 using the Maxwell RSC Blood DNA Kit (Promega #AS1400) and using a Maxwell@RSC48 instrument (Promega #AS8500) according to the manufacturer's instruction. PCR was then performed on the genomic DNA using DMD exon 53-specific primers that targeted the relevant cut site.

### 7. Amplicon Deep Sequencing Library Preparation and Data Analysis

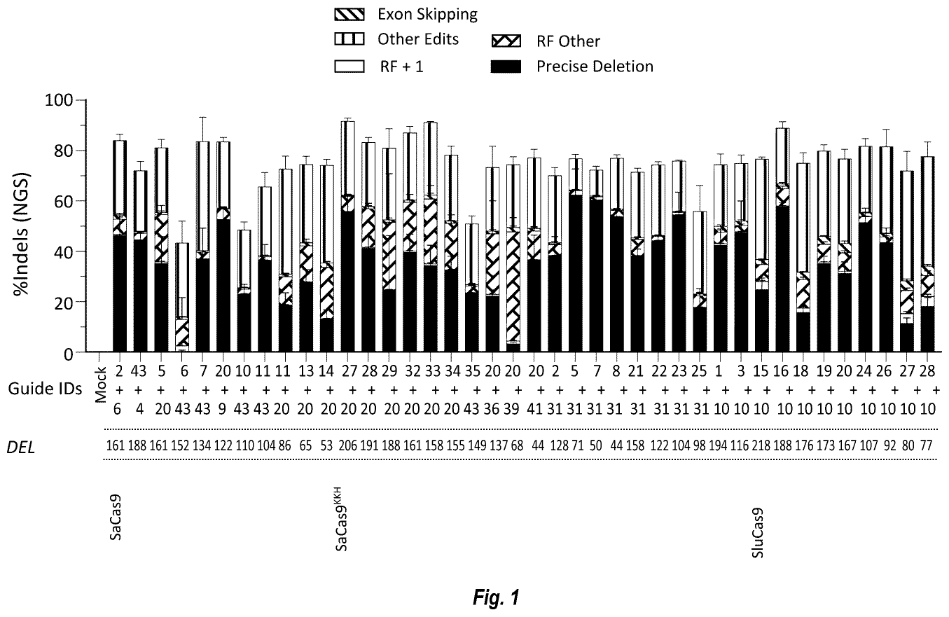

For the data in FIGS. 1-3, Exon 53 or 51 was amplified by PCR and the products were used to prepare sequencing libraries using MiSeq reagent kit V3. Indel analysis was performed using CRISPResso2±10-nt quantification window. (See, e.g., Clement et al., Nat Biotechnol. 2019 March; 37(3):224-226). Indel profiling consisted of six mutually exclusive indel categories, described below:
NE: non-edited;
Precise deletion (i.e. CleanCut);
RF.+1 (i.e. +1 bp): 1-nucleotide (nt) insertion leading to reframe;
RF. Other (i.e. Reframe): indels other than 1-nt insertion leading to reframe:
Deletion: not extending outside of the refraining window
Insertion: <17-nt (i.e., <6 amino acids);
Exon skipping: indels that disrupt the ±6-nt window of the exon/intron boundaries leading to potential exon skipping (outcome requiring validation):
The indel has >=9-nt overlap with the splicing window (to disrupt the GT/AG splicing sites)
OE: Other indels.

The following sequences of selected primers were used for amplification of the specific human locus containing the sgRNA targeting sites (Tables 8A-8B)

TABLE 8A
Primers for Amplification (Human)

| Targeting | Primer | |
|---|---|---|
| exon | ID | Sequence |
| 53 | hEX53_F1 | AAATGTGAGATAACGTTTGGAAG |
| | | (SEQ ID NO: 804) |
| | hEX53_R1 | TTTCAGCTTTAACGTGATTTTCT |
| | | G (SEQ ID NO: 805) |

TABLE 8B
Primer + MiSeq Adapter Sequence (Human)

| Exon | ID | Sequence |
|---|---|---|
| 53 | MiSeq_hEX53_F1 | TCGTCGGCAGCGTCAGATGTGTATAAGA |
| | | GACAGAAATGTGAGATAACGTTTGGAAG |
| | | (SEQ ID NO: 810) |
| | MiSeq_hEX53_R1 | GTCTCGTGGGCTCGGAGATGTGTATAAG |
| | | AGACAGTTTCAGCTTTAACGTGATTTTC |
| | | TG (SEQ ID NO: 811) |

### 8. AAV Configurations for the Dual Cut Single Vector Candidates

A combination of promoter orientations, promoter configurations, NLSs and scaffolds were selected for generating AAV plasmids and evaluation on sgRNA transgene expression, AAV manufacturability, and editing efficiency in vitro and in vivo. AAV plasmid configurations are listed in Table 9.

TABLE 9

| Pol III | | | | | | | |
|---|---|---|---|---|---|---|---|
| Promoter | Orientation | Configuration | NLS1 | Endonuclease | NLS2 | NLS3 | Scaffold |
| hU6c:hU6c | ← [symbol] [symbol] | hU6c-Cas9- | c-Myc- | SluCas9 | SV-40-GSGS | Nucleoplasmin- | V5 |
| | | hU6c | GSVD | | | GSGS | |
| | ← [symbol] [symbol] | hU6c-Cas9- | c-Myc- | SaCas9-KKH | SV-40-GSGS | Nucleoplasmin- | V2 |
| | | hU6c | GSVD | | | GSGS | |
| | ← [symbol] [symbol] | hU6c-Cas9- | c-Myc- | SluCas9 | SV-40-MCS- | Nucleoplasmin- | V5 |
| | | hU6c | GSVD | | GSGS | GSGS | |
| | ← [symbol] [symbol] | hU6c-Cas9- | c-Myc- | SaCas9-KKH | SV-40-MCS- | Nucleoplasmin- | V2 |
| | | hU6c | GSVD | | GSGS | GSGS | |
| hU6:7SK2 | ← [symbol] [symbol] | hU6c-Cas9- | c-Myc- | SluCas9 | SV-40-GSGS | Nucleoplasmin- | V5 |
| | | 7SK2 | GSVD | | | GSGS | |
| | ← [symbol] [symbol] | hU6c-Cas9- | c-Myc- | SaCas9-KKH | SV-40-GSGS | Nucleoplasmin- | V2 |
| | | 7SK2 | GSVD | | | GSGS | |
| | ← [symbol] [symbol] | hU6c-Cas9- | c-Myc- | SluCas9 | SV-40-MCS- | Nucleoplasmin- | V5 |
| | | 7SK2 | GSVD | | GSGS | GSGS | |
| | ← [symbol] [symbol] | hU6c-Cas9- | c-Myc- | SaCas9-KKH | SV-40-MCS- | Nucleoplasmin- | V2 |
| | | 7SK2 | GSVD | | GSGS | GSGS | |
| hU6c:H1m | ← [symbol] [symbol] | hU6c-Cas9- | c-Myc- | SluCas9 | SV-40-GSGS | Nucleoplasmin- | V5 |
| | | H1m | GSVD | | | GSGS | |
| | ← [symbol] [symbol] | hU6c-Cas9- | c-Myc- | SaCas9-KKH | SV-40-GSGS | Nucleoplasmin- | V2 |
| | | H1m | GSVD | | | GSGS | |
| 7SK2:H1m | ← [symbol] [symbol] | 7SK2-Cas9- | SV-40-GS | SluCas9 | Nucleoplasmin | N/A | V5 |
| | | Hlm | | | | | |
| | ← [symbol] [symbol] | 7SK2-Cas9- | SV-40-GS | SaCas9-KKH | Nucleoplasmin | N/A | V2 |
| | | H1m | | | | | |
| | ← [symbol] [symbol] | 7SK2-Cas9- | SV-40 (+) | SluCas9 | Nucleoplasmin | N/A | V5 |
| | | H1m | | | | | |
| | ← [symbol] [symbol] | 7SK2-Cas9- | SV-40 (+) | SaCas9-KKH | Nucleoplasmin | N/A | V2 |
| | | H1m | | | | | |
| | ← [symbol] [symbol] | 7SK2-Cas9- | c-Myc- | SluCas9 | SV-40-MCS- | Nucleoplasmin- | V5 |
| | | H1m | GSVD | | GSGS | GSGS | |
| | ← [symbol] [symbol] | 7SK2-Cas9- | c-Myc- | SaCas9-KKH | SV-40-MCS- | Nucleoplasmin- | V2 |
| | | H1m | GSVD | | GSGS | GSGS | |
| | ← [symbol] [symbol] | 7SK2-Cas9- | c-Myc- | SluCas9 | SV-40-MCS- | Nucleoplasmin- | V2 |
| | | H1m | GSVD | | GSGS | GSGS | |
| H1m:M11 | ← [symbol] [symbol] | H1m-Cas9- | c-Myc- | SluCas9 | SV-40-MCS- | Nucleoplasmin- | V5 |
| | | M11 | GSVD | | GSGS | GSGS | |
| | ← [symbol] [symbol] | H1m-Cas9- | c-Myc- | SaCas9-KKH | SV-40-MCS- | Nucleoplasmin- | V5 |
| | | M11 | GSVD | | GSGS | GSGS | |

Sequences of selected primers used for amplification of the specific human locus containing the sgRNA sites are shown in Table 10.

TABLE 10

| Name | Sequence |
|---|---|
| MiSeq_hE45_F | TCGTCGGCAGCGTCAGATGTGTATAAGAGACAGGTCTTTCTGTCTTGTATCCTTTGG |
| | (SEQ ID NO: 724) |
| MiSeq_hE45_R | GTCTCGTGGGCTCGGAGATGTGTATAAGAGACAGAATGTTAGTGCCTTTCACCC |
| | (SEQ ID NO: 725) |
| TIDE_hE45_F* | GTCTTTCTGTCTTGTATCCTTTGG (SEQ ID NO: 726) |

*TIDE_hE45_F is used for Sanger sequencing.

### 9. Results

A set of sgRNAs found within the DMD gene was selected for evaluation of indel frequency and profile. Among this selection, 4 sgRNAs were located within exon 45, 43 sgRNAs were located within exon 51 and 29 sgRNAs were located within exon 53.

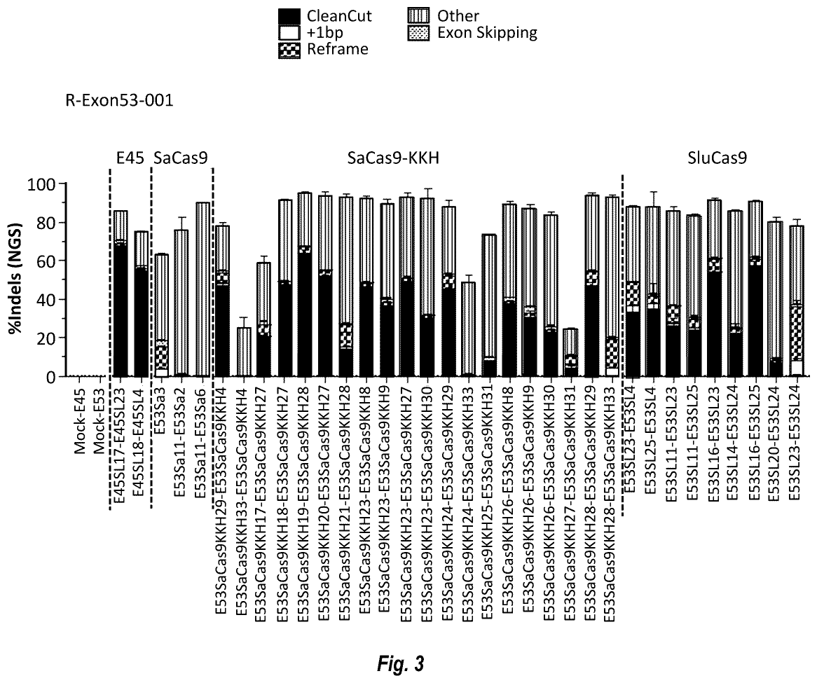

To evaluate indel frequency and editing profiles, plasmid transfection was performed in HEK293FT (FIGS. 1, 3), and Human Skeletal Muscle Myoblasts (HsMM) (FIG. 2A-B).

The editing frequency and indel profile of selected SaCas9, SaCas9-KKH and SluCas9 sgRNA pairs targeting Exon 51 of DMD gene was determined, as shown in FIG. 1. Each stack bar represents a sgRNA pair and the bar height depicts the average frequency of the total edit. Different indel profiles for each pair of sgRNAs are represented by using distinct patterns. Error bars denote the standard deviation for each indel group.

The average indel frequency of sgRNAs targeting exon 51 of the DMD gene was determined in HEK294FT cells (FIG. 1), including precise deletions. The deletion size is shown at bottom.

The average indel frequency of sgRNAs targeting Exon 45 or Exon 51 of the DMD gene was determined in HsMM cells (FIG. 2A-B) as calculated by Inference of CRISPR Edit (ICE) via Sanger sequencing signal decomposition (FIG. 2A) and next generation sequencing (NGS) (FIG. 2B).

The average indel frequency of sgRNAs targeting exon 45 (left) and exon 53 (right) of the DMD gene was determined in HEK294FT cells (FIG. 3), including precise deletions. The deletion size is shown at bottom.

Using a different formulation (see Materials and Methods: Nucleofection of HsMM cells, above), the average indel frequency of sgRNAs targeting Exon 51 of the DMD gene was again determined in HsMM cells (FIGS. 6A-B) as calculated by next generation sequencing (NGS). Based on these results, the following guide pairs were selected for additional characterization: Slu31-Slu5 (SEQ ID NOs: 269 and 247), Slu31-Slu7 (SEQ ID NOs: 269 and 249), Slu10-24 (SEQ ID NOs: 252 and 262), Slu10-26 (SEQ ID NOs: 252 and 264), and Slu10-16 (SEQ ID NOs: 252 and 254).

Nuclease activity of the selected guide pairs was evaluated as a function of dose (FIGS. 7A-B). The results show that Slu31-Slu5 (SEQ ID NOs: 269 and 247) (FIG. 7A) generated the highest precise deletion levels of the selected guide pairs, followed by Slu10-Slu16 (SEQ ID NOs: 252 and 254) (FIG. 7B).

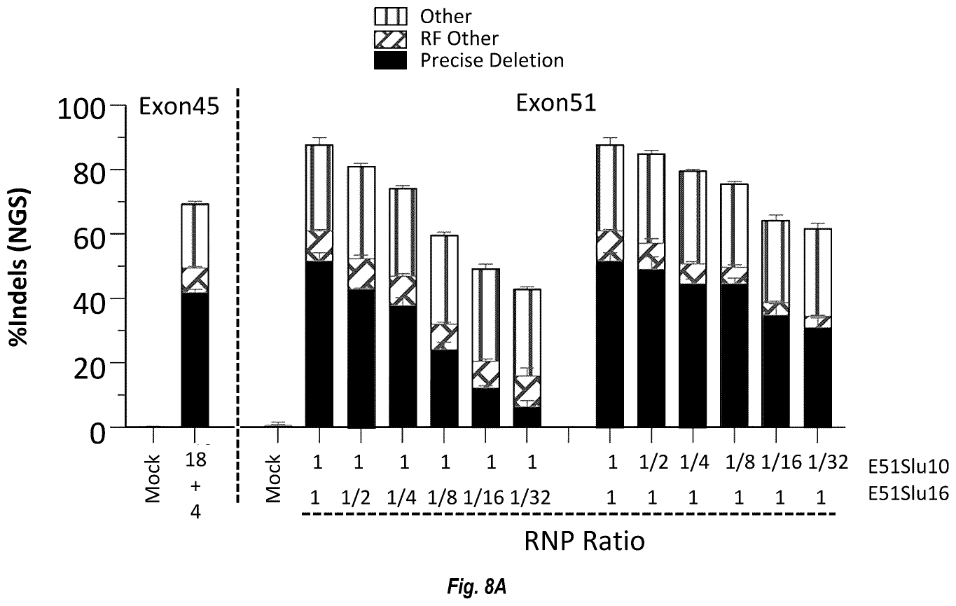

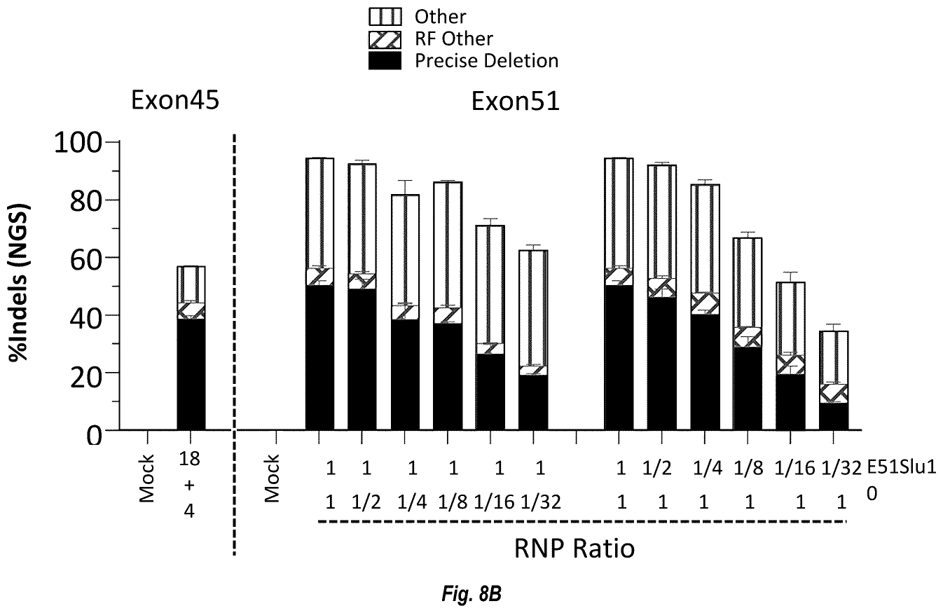

The robustness of the precise deletion regime was evaluated in response to perturbations in RNP stoichiometry (FIGS. 8A-B). The dual cut system appeared more robust for the E51Slu10 (SEQ ID NO: 252) dilution than the E51Slu16 (SEQ ID NO: 254) dilution, suggesting that an AAV configuration with Slu16-Slu10 may be more efficacious at lower doses than several of the other pairs tested. (FIG. 8A). The dual cut system displayed equal robustness for the dilution of either E51Slu10 or E51Slu26 (SEQ ID NO: 264) (FIG. 8B).

### Example 3: Pair Selection for Exon 44 and Exon 50

### A. Materials and Methods

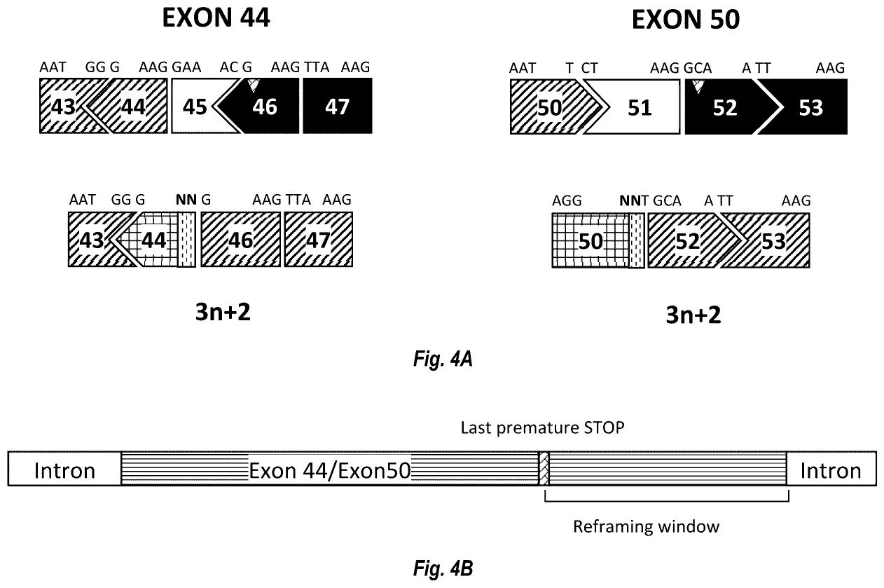

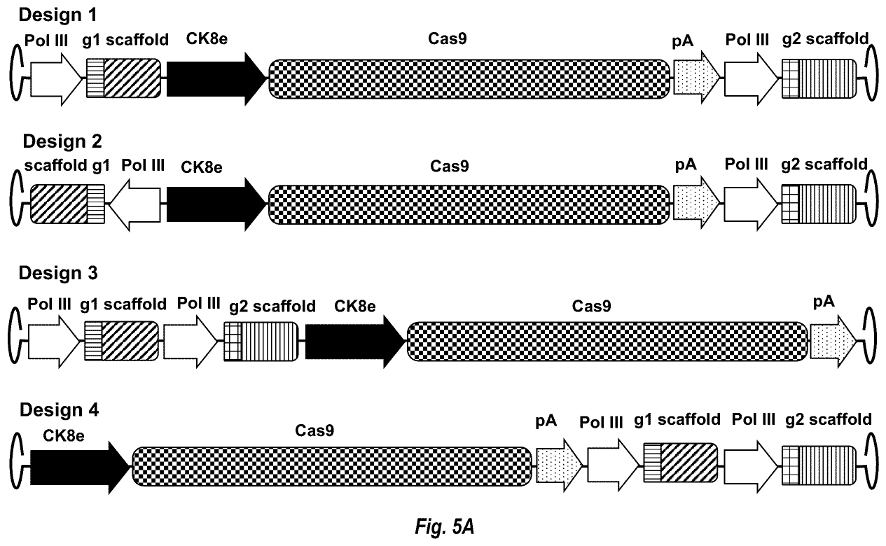

To obtain exemplary guide pairs for exon 44 and exon 50, the refraining window was detected. The refraining window for exon 44 and 50 was defined as the region in which a change in frame would not induce a premature stop codon. The refraining window was calculated by searching premature stop codons in the mutant exon sequence, which was generated by omitting the first base of the exon sequence. Since exon 44 was not in the neutral frame, the searching was conducted from the beginning of exon 43. The window between last premature stop codon and the end of the exon was then determined as the refraining window. Pairs of guide RNAs were then located where the cut-site of at least one guide RNA was within the refraining window. The candidate clean-cut pairs that refrained the exon to a desired frame were a subset of guides for which the cut-distance between the two guides can be represented by the expression “3n+2”. An exemplary reframe is shown in FIGS. 4A and B.

### Example 4: Exon 53 HEK239FT sRGN On-Target Screening

### A. Materials and Methods

### 1. Transfection of HEK293FT Cells

To evaluate indel frequency and profile, human HEK293FT cell lines were used. About 200 k HEK293FT cells were seeded and transfected in each well of 12-well plates, with 750 ng plasmid+2.5 μL of Lipofectamine 2000 per well. Each plasmid expressed the appropriate nuclease and two sgRNAs for dual-cut editing. The Slu_v5 scaffold (SEQ ID NO: 901) was used for all samples, except for one triplicate of a E45Slu18/4 sample. Three days after transfection, cells were trypsinized and sorted for green fluorescent protein (GFP). About 100 k GFP-positive cells were sorted directly into lysis buffer, and DNA extraction was performed using the Maxwell RSC Blood genomic DNA purification kit.

### 2. Amplicon Deep Sequencing, Library Preparation, and Data Analysis

To determine the gene editing efficiency, the genomic DNAs were amplified using primers flanking the DMD exon 53 genomic region. The following primer sequences were used: hEX53_F1 AAATGTGAGATAACGTTTGGAAG (SEQ ID NO: 804) and hEX53_R1 TTTCAGCTTTAACGTGATTTTCTG (SEQ ID NO: 805). The size of the amplicons was verified by analyzing a small amount of the PCR products on 2% E-gels. The PCR product was purified by AMPure XP beads. The purified PCR product was amplified again with primers that contain barcodes and Illumina adaptors: i5_UDP0003 AATGATACGGCGACCACCGAGATCTACACTATAGTAGCTTCGTCGGCAGCGTC (SEQ ID NO: 921), and variable i7 indexing primers CAAGCAGAAGACGGCATACGAGAT[variable_10nt i7_barcode]GTCTCGTGGGCTCGG (CAAGCAGAAGACGGCATACGAGATNNNNNNNNNNGTCTCGTGGGCTCGG, SEQ ID NO: 922). Multiple barcoded samples were pooled, combined with PhiX library, and loaded onto the Illumina Mi-Seq platform. MiSeq Reagent Kit v3 was used to produce a 600-cycle run.

Indel analysis was performed using CRISPResso2±10-nt quantification window. (See, e.g., Clement et al., Nat Biotechnol. 2019 March; 37(3):224-226). Indel profiling was assessed using the mutually exclusive indel categories below:
a) Precise Deletion: precision deletion of the DNA sequence between the predicted cut sites of the sgRNA pairs, which leads to reframe;
b) RF. Other: indels other than precise deletion leading to reframe:
c) OE: Other indels.

### 3. Results

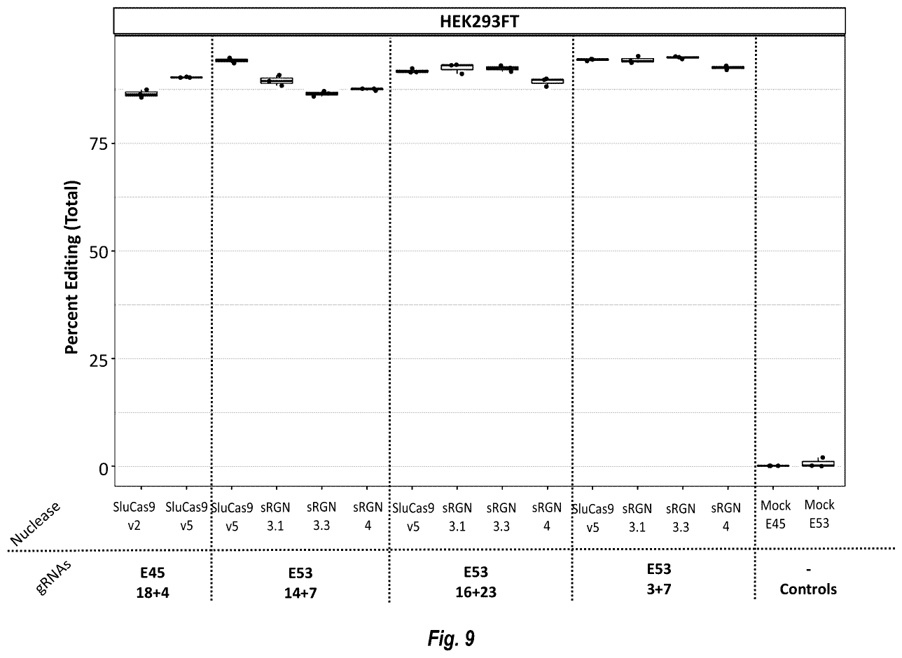

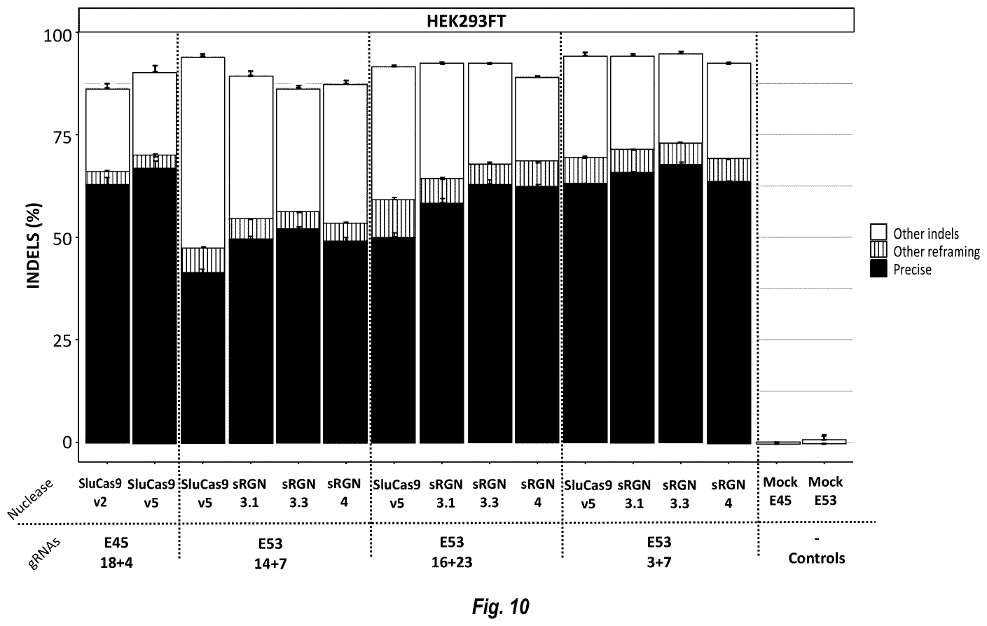

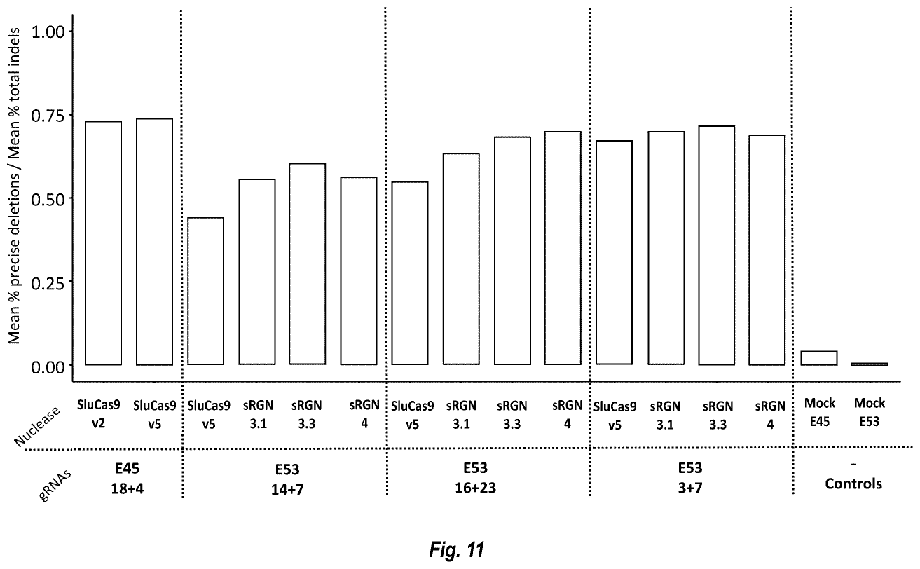

Results of the Exon 53 HEK293FT sRGN on-target screening are shown in FIGS. 9-11. As shown in FIG. 10, the three sRGNs tested (3.1, 3.3, and 4) had higher % precise deletions than SluCas9 for Slu14+7 (SEQ ID NOs: 281 and 275, respectively), Slu 16+23 (SEQ ID NOS: 283 and 290, respectively) and Slu3+7 (SEQ ID NOs: 271 and 275, respectively). Additionally, sRGN3.3 E53SL3+7 showed the highest % precise deletions (67.8%). For each guide pair, sRGN3.3 had the highest % precise deletions.

### Example 5: Exon 53 HsMM sRGN On-Target Screening

### A. Materials and Methods

### 1. Transfection of HsMM Cells

To evaluate indel frequency and profile, primary human skeletal muscle myoblast (HsMM donor number 20TL356515, Lonza) cells were used. Nucleases were delivered as capped mRNA with N1-methylpseudouridine substitutions, produced through in vitro transcription by GenScript. sgRNAs were delivered as modified ssRNA synthesized by Synthego. About 50,000 HsMM cells were seeded and transfected in each well of 12-well plates. In each well, 3000 ng plasmid+0.5 μL of 12.5 M of each sgRNA were added along with Lipofectamine messengerMAX transfection reagent. The Slu_v5 scaffold (SEQ ID NO: 901) was used for all sgRNAs. Two days after transfection, cells were trypsinized, and genomic DNA extraction was performed using the Maxwell RSC Blood genomic DNA purification kit.

### 2. Amplicon Deep Sequencing, Library Preparation, and Data Analysis

To determine the gene editing efficiency, the genomic DNAs were amplified using primers flanking the DMD exon 53 genomic region. The following primer sequences were used: hEX53_F1 AAATGTGAGATAACGTTTGGAAG (SEQ ID NO: 804) and hEX53_R1 TTTCAGCTTTAACGTGATTTTCTG (SEQ ID NO: 805). The size of the amplicons was verified by analyzing a small amount of the PCR products on 2% E-gels. The PCR product was purified using AMPure XP beads. The purified PCR product was amplified again with primers that contain barcodes and Illumina adaptors: i5_UDP0003 AATGATACGGCGACCACCGAGATCTACACTATAGTAGCTTCGTCGGCAGCGTC (SEQ ID NO: 921), and variable i7 indexing primers CAAGCAGAAGACGGCATACGAGAT[variable_10nt_i7_barcode]GTCTCGTGGGCTCGG (CAAGCAGAAGACGGCATACGAGATNNNNNNNNNNGTCTCGTGGGCTCGG, SEQ ID NO: 922). Multiple barcoded samples were pooled, combined with PhiX library, and loaded onto the Illumina Mi-Seq platform. MiSeq Reagent Kit v3 was used to produce a 600-cycle run.

Indel analysis was performed using CRISPResso2±10-nt quantification window (See Clement et al., Nat Biotechnol. 2019 March; 37(3):224-226). Indel profiling was assessed using the mutually exclusive indel categories below:
a) Precise Deletion: precision deletion of the DNA sequence between the predicted cut sites of the sgRNA pairs, which leads to reframe;
b) RF. Other: indels other than precise deletion leading to reframe:
c) OE: Other indels.

### 3. Results

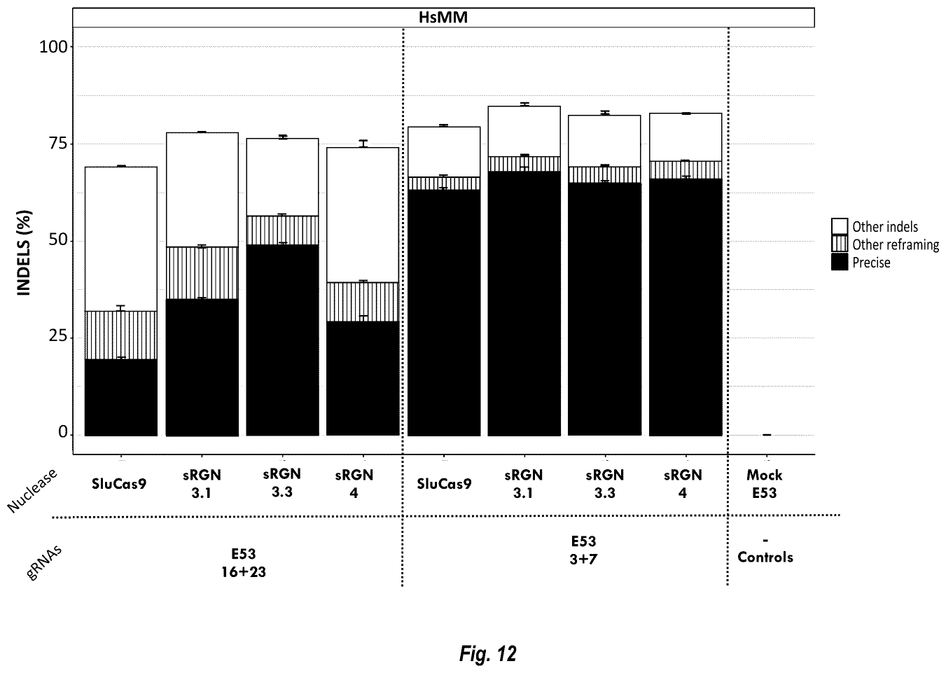

Results of the Exon 53 HsMM sRGN on-target screening are shown in FIG. 12. The three sRGNs tested (3.1, 3.3, and 4) exhibited higher % precise deletions than SluCas9 for both Slu16+23 (SEQ ID NOs: 283 and 290, respectively) and Slu3+7 (SEQ ID NOs: 271 and 275, respectively), with increases in % precise deletions greatest for the three sRGNs with Slu16+23 as compared to SluCas9 with Slu16+23.

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

1. A composition comprising one or more guide RNAs or a nucleic acid encoding one or more guide RNAs, wherein the one or more guide RNAs comprise i) a guide sequence of Table 6; ii) at least 16, 17, 18, 19, or 20 contiguous nucleotides of a guide sequence of Table 6; iii) a guide sequence that is at least 90% identical to a guide sequence of Table 6; or iv) any one of the guide sequence pairs shown in Tables 1B, 1D, 3B, 3D, 5B, and 5D, optionally further comprising a SaCas9 or a nucleic acid encoding a SaCas9 (for SEQ ID NOs: 1-159) or a SluCas9 or a nucleic acid encoding a SluCas9 (for SEQ ID NOs: 200-292, 924-938, or 950-955).

2. A composition comprising a pair of guide RNAs or a nucleic acid encoding a pair of guide RNAs, wherein the pair of guide RNAs comprise or consist of a first and a second guide sequence, wherein the first and the second guide sequences are selected from any of the following pairs of guide sequences:
a. For exon 44 with SaCas9, SEQ ID NOs: 1 and 3; 110 and 120; 110 and 121; 110 and 122; 110 and 123; 110 and 124; 110 and 125; 111 and 112; 111 and 113; 111 and 114; 111 and 115; 111 and 116; 111 and 117; 111 and 118; 111 and 119; 111 and 120; 111 and 121; 111 and 122; 111 and 123; 111 and 124; 111 and 125; 112 and 113; 112 and 114; 112 and 115; 112 and 116; 112 and 117; 112 and 118; 112 and 119; 112 and 120; 112 and 121; 112 and 122; 112 and 123; 112 and 124; 112 and 125; 113 and 114; 113 and 115; 113 and 116; 113 and 117; 113 and 118; 113 and 119; 113 and 120; 113 and 121; 113 and 122; 113 and 123; 113 and 124; 113 and 125; 114 and 115; 114 and 116; 114 and 117; 114 and 118; 114 and 119; 114 and 120; 114 and 121; 114 and 122; 114 and 123; 114 and 124; 114 and 125; 115 and 116; 115 and 117; 115 and 118; 115 and 119; 115 and 120; 115 and 121; 115 and 122; 115 and 123; 115 and 124; 115 and 125; 116 and 117; 116 and 118; 116 and 119; 116 and 120; 116 and 121; 116 and 122; 116 and 123; 116 and 124; 116 and 125; 117 and 118; 117 and 119; 117 and 120; 117 and 121; 117 and 122; 117 and 123; 117 and 124; 117 and 125; 118 and 119; 118 and 120; 118 and 121; 118 and 122; 118 and 123; 118 and 124; 118 and 125; 119 and 120; 119 and 121; 119 and 122; 119 and 123; 119 and 124; 119 and 125; 120 and 121; 120 and 122; 120 and 123; 120 and 124; 120 and 125; 121 and 122; 121 and 123; 121 and 124; 121 and 125; 122 and 123; 122 and 124; 122 and 125; 123 and 124; 123 and 125; or 124 and 125;
b. For exon 44 with SluCas9, SEQ ID NOs: 200 and 201; 200 and 202; 200 and 203; 200 and 204; 200 and 205; 200 and 206; 200 and 207; 200 and 208; 201 and 202; 201 and 203; 201 and 204; 201 and 205; 201 and 206; 201 and 207; 201 and 208; 202 and 203; 202 and 204; 202 and 205; 202 and 206; 202 and 207; 202 and 208; 203 and 204; 203 and 205; 203 and 206; 203 and 207; 203 and 208; 204 and 205; 204 and 206; 204 and 207; 204 and 208; 205 and 206; 205 and 207; 205 and 208; 206 and 207; 206 and 208; or 207 and 208;
c. For exon 50 with SaCas9, SEQ ID NOs: 148 and 149; 148 and 150; 148 and 151; 148 and 152; 148 and 153; 148 and 154; 148 and 155; 148 and 156; 148 and 157; 148 and 158; 148 and 159; 149 and 150; 149 and 151; 149 and 152; 149 and 153; 149 and 154; 149 and 155; 149 and 156; 149 and 157; 149 and 158; 149 and 159; 150 and 151; 150 and 152; 150 and 153; 150 and 154; 150 and 155; 150 and 156; 150 and 157; 150 and 158; 150 and 159; 151 and 152; 151 and 153; 151 and 154; 151 and 155; 151 and 156; 151 and 157; 151 and 158; 151 and 159; 152 and 153; 152 and 154; 152 and 155; 152 and 156; 152 and 157; 152 and 158; 152 and 159; 153 and 154; 153 and 155; 153 and 156; 153 and 157; 153 and 158; 153 and 159; 154 and 155; 154 and 156; 154 and 157; 154 and 158; 154 and 159; 155 and 156; 155 and 157; 155 and 158; 155 and 159; 156 and 157; 156 and 158; 156 and 159; 157 and 158; 157 and 159; or 158 and 159;
d. For exon 50 with SluCas9, SEQ ID NOs: 231 and 232; 231 and 233; 231 and 234; 231 and 235; 231 and 236; 231 and 237; 231 and 238; 231 and 239; 231 and 240; 231 and 241; 231 and 242; 232 and 233; 232 and 234; 232 and 235; 232 and 236; 232 and 237; 232 and 238; 232 and 239; 232 and 240; 232 and 241; 232 and 242; 233 and 234; 233 and 235; 233 and 236; 233 and 237; 233 and 238; 233 and 239; 233 and 240; 233 and 241; 233 and 242; 234 and 235; 234 and 236; 234 and 237; 234 and 238; 234 and 239; 234 and 240; 234 and 241; 234 and 242; 235 and 236; 235 and 237; 235 and 238; 235 and 239; 235 and 240; 235 and 241; 235 and 242; 236 and 237; 236 and 238; 236 and 239; 236 and 240; 236 and 241; 236 and 242; 237 and 238; 237 and 239; 237 and 240; 237 and 241; 237 and 242; 238 and 239; 238 and 240; 238 and 241; 238 and 242; 239 and 240; 239 and 241; 239 and 242; 240 and 241; 240 and 242; or 241 and 242;
e. For exon 53 with SaCas9, SEQ ID NOs: 16 and 17; 16 and 18; 16 and 19; 16 and 20; 16 and 21; 16 and 22; 16 and 23; 16 and 24; 16 and 25; 16 and 26; 17 and 18; 17 and 19; 17 and 20; 17 and 21; 17 and 22; 17 and 23; 17 and 24; 17 and 25; 17 and 26; 18 and 19; 18 and 20; 18 and 21; 18 and 22; 18 and 23; 18 and 24; 18 and 25; 18 and 26; 19 and 20; 19 and 21; 19 and 22; 19 and 23; 19 and 24; 19 and 25; 19 and 26; 20 and 21; 20 and 22; 20 and 23; 20 and 24; 20 and 25; 20 and 26; 21 and 22; 21 and 23; 21 and 24; 21 and 25; 21 and 26; 22 and 23; 22 and 24; 22 and 25; 22 and 26; 23 and 24; 23 and 25; 23 and 26; 24 and 25; 24 and 26; 25 and 26; 71 and 72; 71 and 73; 71 and 74; 71 and 75; 71 and 76; 71 and 77; 71 and 78; 71 and 84; 71 and 85; 71 and 86; 71 and 87; 71 and 88; 71 and 89; 71 and 90; 71 and 91; 71 and 92; 71 and 93; 71 and 94; 71 and 95; 71 and 96; 71 and 97; 71 and 98; 71 and 99; 71 and 100; 71 and 101; 71 and 102; 72 and 73; 72 and 74; 72 and 75; 72 and 76; 72 and 77; 72 and 78; 72 and 84; 72 and 85; 72 and 86; 72 and 87; 72 and 88; 72 and 89; 72 and 90; 72 and 91; 72 and 92; 72 and 93; 72 and 94; 72 and 95; 72 and 96; 72 and 97; 72 and 98; 72 and 99; 72 and 100; 72 and 101; 72 and 102; 73 and 74; 73 and 75; 73 and 76; 73 and 77; 73 and 78; 73 and 84; 73 and 85; 73 and 86; 73 and 87; 73 and 88; 73 and 89; 73 and 90; 73 and 91; 73 and 92; 73 and 93; 73 and 94; 73 and 95; 73 and 96; 73 and 97; 73 and 98; 73 and 99; 73 and 100; 73 and 101; 73 and 102; 74 and 75; 74 and 76; 74 and 77; 74 and 78; 74 and 84; 74 and 85; 74 and 86; 74 and 87; 74 and 88; 74 and 89; 74 and 90; 74 and 91; 74 and 92; 74 and 93; 74 and 94; 74 and 95; 74 and 96; 74 and 97; 74 and 98; 74 and 99; 74 and 100; 74 and 101; 74 and 102; 75 and 76; 75 and 77; 75 and 78; 75 and 84; 75 and 85; 75 and 86; 75 and 87; 75 and 88; 75 and 89; 75 and 90; 75 and 91; 75 and 92; 75 and 93; 75 and 94; 75 and 95; 75 and 96; 75 and 97; 75 and 98; 75 and 99; 75 and 100; 75 and 101; 75 and 102; 76 and 77; 76 and 78; 76 and 84; 76 and 85; 76 and 86; 76 and 87; 76 and 88; 76 and 89; 76 and 90; 76 and 91; 76 and 92; 76 and 93; 76 and 94; 76 and 95; 76 and 96; 76 and 97; 76 and 98; 76 and 99; 76 and 100; 76 and 101; 76 and 102; 77 and 78; 77 and 84; 77 and 85; 77 and 86; 77 and 87; 77 and 88; 77 and 89; 77 and 90; 77 and 91; 77 and 92; 77 and 93; 77 and 94; 77 and 95; 77 and 96; 77 and 97; 77 and 98; 77 and 99; 77 and 100; 77 and 101; 77 and 102; 78 and 84; 78 and 85; 78 and 86; 78 and 87; 78 and 88; 78 and 89; 78 and 90; 78 and 91; 78 and 92; 78 and 93; 78 and 94; 78 and 95; 78 and 96; 78 and 97; 78 and 98; 78 and 99; 78 and 100; 78 and 101; 78 and 102; 84 and 85; 84 and 86; 84 and 87; 84 and 88; 84 and 89; 84 and 90; 84 and 91; 84 and 92; 84 and 93; 84 and 94; 84 and 95; 84 and 96; 84 and 97; 84 and 98; 84 and 99; 84 and 100; 84 and 101; 84 and 102; 85 and 86; 85 and 87; 85 and 88; 85 and 89; 85 and 90; 85 and 91; 85 and 92; 85 and 93; 85 and 94; 85 and 95; 85 and 96; 85 and 97; 85 and 98; 85 and 99; 85 and 100; 85 and 101; 85 and 102; 86 and 87; 86 and 88; 86 and 89; 86 and 90; 86 and 91; 86 and 92; 86 and 93; 86 and 94; 86 and 95; 86 and 96; 86 and 97; 86 and 98; 86 and 99; 86 and 100; 86 and 101; 86 and 102; 87 and 88; 87 and 89; 87 and 90; 87 and 91; 87 and 92; 87 and 93; 87 and 94; 87 and 95; 87 and 96; 87 and 97; 87 and 98; 87 and 99; 87 and 100; 87 and 101; 87 and 102; 88 and 89; 88 and 90; 88 and 91; 88 and 92; 88 and 93; 88 and 94; 88 and 95; 88 and 96; 88 and 97; 88 and 98; 88 and 99; 88 and 100; 88 and 101; 88 and 102; 89 and 90; 89 and 91; 89 and 92; 89 and 93; 89 and 94; 89 and 95; 89 and 96; 89 and 97; 89 and 98; 89 and 99; 89 and 100; 89 and 101; 89 and 102; 90 and 91; 90 and 92; 90 and 93; 90 and 94; 90 and 95; 90 and 96; 90 and 97; 90 and 98; 90 and 99; 90 and 100; 90 and 101; 90 and 102; 91 and 92; 91 and 93; 91 and 94; 91 and 95; 91 and 96; 91 and 97; 91 and 98; 91 and 99; 91 and 100; 91 and 101; 91 and 102; 92 and 93; 92 and 94; 92 and 95; 92 and 96; 92 and 97; 92 and 98; 92 and 99; 92 and 100; 92 and 101; 92 and 102; 93 and 94; 93 and 95; 93 and 96; 93 and 97; 93 and 98; 93 and 99; 93 and 100; 93 and 101; 93 and 102; 94 and 95; 94 and 96; 94 and 97; 94 and 98; 94 and 99; 94 and 100; 94 and 101; 94 and 102; 95 and 96; 95 and 97; 95 and 98; 95 and 99; 95 and 100; 95 and 101; 95 and 102; 96 and 97; 96 and 98; 96 and 99; 96 and 100; 96 and 101; 96 and 102; 97 and 98; 97 and 99; 97 and 100; 97 and 101; 97 and 102; 98 and 99; 98 and 100; 98 and 101; 98 and 102; 99 and 100; 99 and 101; 99 and 102; 100 and 101; 100 and 102; or 101 and 102;
f. For exon 53 with SluCas9, SEQ ID NOs: 270 and 271; 270 and 272; 270 and 273; 270 and 274; 270 and 275; 270 and 276; 270 and 277; 270 and 278; 270 and 279; 270 and 280; 270 and 281; 270 and 282; 270 and 283; 270 and 284; 270 and 285; 270 and 286; 270 and 287; 270 and 288; 270 and 289; 270 and 290; 270 and 291; 270 and 292; 271 and 272; 271 and 273; 271 and 274; 271 and 275; 271 and 276; 271 and 277; 271 and 278; 271 and 279; 271 and 280; 271 and 281; 271 and 282; 271 and 283; 271 and 284; 271 and 285; 271 and 286; 271 and 287; 271 and 288; 271 and 289; 271 and 290; 271 and 291; 271 and 292; 272 and 273; 272 and 274; 272 and 275; 272 and 276; 272 and 277; 272 and 278; 272 and 279; 272 and 280; 272 and 281; 272 and 282; 272 and 283; 272 and 284; 272 and 285; 272 and 286; 272 and 287; 272 and 288; 272 and 289; 272 and 290; 272 and 291; 272 and 292; 273 and 274; 273 and 275; 273 and 276; 273 and 277; 273 and 278; 273 and 279; 273 and 280; 273 and 281; 273 and 282; 273 and 283; 273 and 284; 273 and 285; 273 and 286; 273 and 287; 273 and 288; 273 and 289; 273 and 290; 273 and 291; 273 and 292; 274 and 275; 274 and 276; 274 and 277; 274 and 278; 274 and 279; 274 and 280; 274 and 281; 274 and 282; 274 and 283; 274 and 284; 274 and 285; 274 and 286; 274 and 287; 274 and 288; 274 and 289; 274 and 290; 274 and 291; 274 and 292; 275 and 276; 275 and 277; 275 and 278; 275 and 279; 275 and 280; 275 and 281; 275 and 282; 275 and 283; 275 and 284; 275 and 285; 275 and 286; 275 and 287; 275 and 288; 275 and 289; 275 and 290; 275 and 291; 275 and 292; 276 and 277; 276 and 278; 276 and 279; 276 and 280; 276 and 281; 276 and 282; 276 and 283; 276 and 284; 276 and 285; 276 and 286; 276 and 287; 276 and 288; 276 and 289; 276 and 290; 276 and 291; 276 and 292; 277 and 278; 277 and 279; 277 and 280; 277 and 281; 277 and 282; 277 and 283; 277 and 284; 277 and 285; 277 and 286; 277 and 287; 277 and 288; 277 and 289; 277 and 290; 277 and 291; 277 and 292; 278 and 279; 278 and 280; 278 and 281; 278 and 282; 278 and 283; 278 and 284; 278 and 285; 278 and 286; 278 and 287; 278 and 288; 278 and 289; 278 and 290; 278 and 291; 278 and 292; 279 and 280; 279 and 281; 279 and 282; 279 and 283; 279 and 284; 279 and 285; 279 and 286; 279 and 287; 279 and 288; 279 and 289; 279 and 290; 279 and 291; 279 and 292; 280 and 281; 280 and 282; 280 and 283; 280 and 284; 280 and 285; 280 and 286; 280 and 287; 280 and 288; 280 and 289; 280 and 290; 280 and 291; 280 and 292; 281 and 282; 281 and 283; 281 and 284; 281 and 285; 281 and 286; 281 and 287; 281 and 288; 281 and 289; 281 and 290; 281 and 291; 281 and 292; 282 and 283; 282 and 284; 282 and 285; 282 and 286; 282 and 287; 282 and 288; 282 and 289; 282 and 290; 282 and 291; 282 and 292; 283 and 284; 283 and 285; 283 and 286; 283 and 287; 283 and 288; 283 and 289; 283 and 290; 283 and 291; 283 and 292; 284 and 285; 284 and 286; 284 and 287; 284 and 288; 284 and 289; 284 and 290; 284 and 291; 284 and 292; 285 and 286; 285 and 287; 285 and 288; 285 and 289; 285 and 290; 285 and 291; 285 and 292; 286 and 287; 286 and 288; 286 and 289; 286 and 290; 286 and 291; 286 and 292; 287 and 288; 287 and 289; 287 and 290; 287 and 291; 287 and 292; 288 and 289; 288 and 290; 288 and 291; 288 and 292; 289 and 290; 289 and 291; 289 and 292; 290 and 291; 290 and 292; or 291 and 292; 924 and 270; 924 and 272; 924 and 273; 924 and 274; 924 and 275; 924 and 276; 924 and 277; 924 and 278; 924 and 279; 924 and 280; 924 and 281; 924 and 282; 924 and 283; 924 and 284; 924 and 285; 924 and 286; 924 and 287; 924 and 288; 924 and 289; 924 and 290; 924 and 291; 924 and 292; 925 and 270; 925 and 272; 925 and 273; 925 and 274; 925 and 275; 925 and 276; 925 and 277; 925 and 278; 925 and 279; 925 and 280; 925 and 281; 925 and 282; 925 and 283; 925 and 284; 925 and 285; 925 and 286; 925 and 287; 925 and 288; 925 and 289; 925 and 290; 925 and 291; 925 and 292; 926 and 270; 926 and 272; 926 and 273; 926 and 274; 926 and 275; 926 and 276; 926 and 277; 926 and 278; 926 and 279; 926 and 280; 926 and 281; 926 and 282; 926 and 283; 926 and 284; 926 and 285; 926 and 286; 926 and 287; 926 and 288; 926 and 289; 926 and 290; 926 and 291; 926 and 292; 927 and 270; 927 and 272; 927 and 273; 927 and 274; 927 and 275; 927 and 276; 927 and 277; 927 and 278; 927 and 279; 927 and 280; 927 and 281; 927 and 282; 927 and 283; 927 and 284; 927 and 285; 927 and 286; 927 and 287; 927 and 288; 927 and 289; 927 and 290; 927 and 291; 927 and 292; 928 and 270; 928 and 272; 928 and 273; 928 and 274; 928 and 275; 928 and 276; 928 and 277; 928 and 278; 928 and 279; 928 and 280; 928 and 281; 928 and 282; 928 and 283; 928 and 284; 928 and 285; 928 and 286; 928 and 287; 928 and 288; 928 and 289; 928 and 290; 928 and 291; 928 and 292; 929 and 270; 929 and 272; 929 and 273; 929 and 274; 929 and 275; 929 and 276; 929 and 277; 929 and 278; 929 and 279; 929 and 280; 929 and 281; 929 and 282; 929 and 283; 929 and 284; 929 and 285; 929 and 286; 929 and 287; 929 and 288; 929 and 289; 929 and 290; 929 and 291; 929 and 292; 930 and 270; 930 and 272; 930 and 273; 930 and 274; 930 and 275; 930 and 276; 930 and 277; 930 and 278; 930 and 279; 930 and 280; 930 and 281; 930 and 282; 930 and 283; 930 and 284; 930 and 285; 930 and 286; 930 and 287; 930 and 288; 930 and 289; 930 and 290; 930 and 291; 930 and 292; 931 and 270; 931 and 272; 931 and 273; 931 and 274; 931 and 275; 931 and 276; 931 and 277; 931 and 278; 931 and 279; 931 and 280; 931 and 281; 931 and 282; 931 and 283; 931 and 284; 931 and 285; 931 and 286; 931 and 287; 931 and 288; 931 and 289; 931 and 290; 931 and 291; 931 and 292; 932 and 270; 932 and 272; 932 and 273; 932 and 274; 932 and 275; 932 and 276; 932 and 277; 932 and 278; 932 and 279; 932 and 280; 932 and 281; 932 and 282; 932 and 283; 932 and 284; 932 and 285; 932 and 286; 932 and 287; 932 and 288; 932 and 289; 932 and 290; 932 and 291; 932 and 292; 933 and 270; 933 and 272; 933 and 273; 933 and 274; 933 and 275; 933 and 276; 933 and 277; 933 and 278; 933 and 279; 933 and 280; 933 and 281; 933 and 282; 933 and 283; 933 and 284; 933 and 285; 933 and 286; 933 and 287; 933 and 288; 933 and 289; 933 and 290; 933 and 291; 933 and 292; 934 and 270; 934 and 272; 934 and 273; 934 and 274; 934 and 275; 934 and 276; 934 and 277; 934 and 278; 934 and 279; 934 and 280; 934 and 281; 934 and 282; 934 and 283; 934 and 284; 934 and 285; 934 and 286; 934 and 287; 934 and 288; 934 and 289; 934 and 290; 934 and 291; 934 and 292; 935 and 270; 935 and 272; 935 and 273; 935 and 274; 935 and 275; 935 and 276; 935 and 277; 935 and 278; 935 and 279; 935 and 280; 935 and 281; 935 and 282; 935 and 283; 935 and 284; 935 and 285; 935 and 286; 935 and 287; 935 and 288; 935 and 289; 935 and 290; 935 and 291; 935 and 292; 936 and 270; 936 and 272; 936 and 273; 936 and 274; 936 and 275; 936 and 276; 936 and 277; 936 and 278; 936 and 279; 936 and 280; 936 and 281; 936 and 282; 936 and 283; 936 and 284; 936 and 285; 936 and 286; 936 and 287; 936 and 288; 936 and 289; 936 and 290; 936 and 291; 936 and 292; 937 and 270; 937 and 272; 937 and 273; 937 and 274; 937 and 275; 937 and 276; 937 and 277; 937 and 278; 937 and 279; 937 and 280; 937 and 281; 937 and 282; 937 and 283; 937 and 284; 937 and 285; 937 and 286; 937 and 287; 937 and 288; 937 and 289; 937 and 290; 937 and 291; 937 and 292; 938 and 270; 938 and 272; 938 and 273; 938 and 274; 938 and 275; 938 and 276; 938 and 277; 938 and 278; 938 and 279; 938 and 280; 938 and 281; 938 and 282; 938 and 283; 938 and 284; 938 and 285; 938 and 286; 938 and 287; 938 and 288; 938 and 289; 938 and 290; 938 and 291; 938 and 292; 950 and 275; 951 and 275; 952 and 275; 953 and 275; 954 and 275; or 955 and 275;
g. For exon 44 with SluCas9, SEQ ID NOs: 200 and 203; 200 and 204; 202 and 203; 202 and 205; 202 and 206; 202 and 207; 204 and 208; 204 and 205; 204 and 206; 204 and 207; or 204 and 208;
h. For exon 50 with SluCas9, SEQ ID NOs: 231 and 232; 231 and 234; 231 and 236; 231 and 237; 236 and 233; 236 and 235; 236 and 238; 236 and 240; or 236 and 241;
i. For exon 53 with SaCas9, SEQ ID NOs: 86 and 96; 87 and 96; 88 and 97; 89 and 96; 90 and 97; 92 and 77; 92 and 78; 92 and 96; 92 and 99; 93 and 98; 93 and 102; 94 and 100; 95 and 77; 95 and 78; 95 and 99; 96 and 100; 97 and 98; 97 and 102; 98 and 73; or 102 and 73; and
j. For exon 53 with SluCas9, SEQ ID NOs: 278 and 290; 278 and 292; 281 and 291; 283 and 290; 283 and 292; 287 and 291; 272 and 290; 290 and 291; or 272 and 292.

3. A composition comprising: one or more nucleic acid molecules encoding
a. a SaCas9 or SluCas9; and
b. a first guide sequence and a second guide sequence, wherein the first and the second guide sequences are selected from any of the following pairs of guide sequences: SEQ ID NO: 1 and SEQ ID NO: 3; SEQ ID NO: 16 and SEQ ID NO: 17; SEQ ID NO: 16 and SEQ ID NO: 18; SEQ ID NO: 16 and SEQ ID NO: 19; SEQ ID NO: 16 and SEQ ID NO: 20; SEQ ID NO: 16 and SEQ ID NO: 21; SEQ ID NO: 16 and SEQ ID NO: 22; SEQ ID NO: 16 and SEQ ID NO: 23; SEQ ID NO: 16 and SEQ ID NO: 24; SEQ ID NO: 16 and SEQ ID NO: 25; SEQ ID NO: 16 and SEQ ID NO: 26; SEQ ID NO: 17 and SEQ ID NO: 18; SEQ ID NO: 17 and SEQ ID NO: 19; SEQ ID NO: 17 and SEQ ID NO: 20; SEQ ID NO: 17 and SEQ ID NO: 21; SEQ ID NO: 17 and SEQ ID NO: 22; SEQ ID NO: 17 and SEQ ID NO: 23; SEQ ID NO: 17 and SEQ ID NO: 24; SEQ ID NO: 17 and SEQ ID NO: 25; SEQ ID NO: 17 and SEQ ID NO: 26; SEQ ID NO: 18 and SEQ ID NO: 19; SEQ ID NO: 18 and SEQ ID NO: 20; SEQ ID NO: 18 and SEQ ID NO: 21; SEQ ID NO: 18 and SEQ ID NO: 22; SEQ ID NO: 18 and SEQ ID NO: 23; SEQ ID NO: 18 and SEQ ID NO: 24; SEQ ID NO: 18 and SEQ ID NO: 25; SEQ ID NO: 18 and SEQ ID NO: 26; SEQ ID NO: 19 and SEQ ID NO: 20; SEQ ID NO: 19 and SEQ ID NO: 21; SEQ ID NO: 19 and SEQ ID NO: 22; SEQ ID NO: 19 and SEQ ID NO: 23; SEQ ID NO: 19 and SEQ ID NO: 24; SEQ ID NO: 19 and SEQ ID NO: 25; SEQ ID NO: 19 and SEQ ID NO: 26; SEQ ID NO: 20 and SEQ ID NO: 21; SEQ ID NO: 20 and SEQ ID NO: 22; SEQ ID NO: 20 and SEQ ID NO: 23; SEQ ID NO: 20 and SEQ ID NO: 24; SEQ ID NO: 20 and SEQ ID NO: 25; SEQ ID NO: 20 and SEQ ID NO: 26; SEQ ID NO: 21 and SEQ ID NO: 22; SEQ ID NO: 21 and SEQ ID NO: 23; SEQ ID NO: 21 and SEQ ID NO: 24; SEQ ID NO: 21 and SEQ ID NO: 25; SEQ ID NO: 21 and SEQ ID NO: 26; SEQ ID NO: 22 and SEQ ID NO: 23; SEQ ID NO: 22 and SEQ ID NO: 24; SEQ ID NO: 22 and SEQ ID NO: 25; SEQ ID NO: 22 and SEQ ID NO: 26; SEQ ID NO: 23 and SEQ ID NO: 24; SEQ ID NO: 23 and SEQ ID NO: 25; SEQ ID NO: 23 and SEQ ID NO: 26; SEQ ID NO: 24 and SEQ ID NO: 25; SEQ ID NO: 24 and SEQ ID NO: 26; SEQ ID NO: 25 and SEQ ID NO: 26; SEQ ID NO: 110 and SEQ ID NO: 120; SEQ ID NO: 110 and SEQ ID NO: 121; SEQ ID NO: 110 and SEQ ID NO: 122; SEQ ID NO: 110 and SEQ ID NO: 123; SEQ ID NO: 110 and SEQ ID NO: 124; SEQ ID NO: 110 and SEQ ID NO: 125; SEQ ID NO: 111 and SEQ ID NO: 112; SEQ ID NO: 111 and SEQ ID NO: 113; SEQ ID NO: 111 and SEQ ID NO: 114; SEQ ID NO: 111 and SEQ ID NO: 115; SEQ ID NO: 111 and SEQ ID NO: 116; SEQ ID NO: 111 and SEQ ID NO: 117; SEQ ID NO: 111 and SEQ ID NO: 118; SEQ ID NO: 111 and SEQ ID NO: 119; SEQ ID NO: 111 and SEQ ID NO: 120; SEQ ID NO: 111 and SEQ ID NO: 121; SEQ ID NO: 111 and SEQ ID NO: 122; SEQ ID NO: 111 and SEQ ID NO: 123; SEQ ID NO: 111 and SEQ ID NO: 124; SEQ ID NO: 111 and SEQ ID NO: 125; SEQ ID NO: 112 and SEQ ID NO: 113; SEQ ID NO: 112 and SEQ ID NO: 114; SEQ ID NO: 112 and SEQ ID NO: 115; SEQ ID NO: 112 and SEQ ID NO: 116; SEQ ID NO: 112 and SEQ ID NO: 117; SEQ ID NO: 112 and SEQ ID NO: 118; SEQ ID NO: 112 and SEQ ID NO: 119; SEQ ID NO: 112 and SEQ ID NO: 120; SEQ ID NO: 112 and SEQ ID NO: 121; SEQ ID NO: 112 and SEQ ID NO: 122; SEQ ID NO: 112 and SEQ ID NO: 123; SEQ ID NO: 112 and SEQ ID NO: 124; SEQ ID NO: 112 and SEQ ID NO: 125; SEQ ID NO: 113 and SEQ ID NO: 114; SEQ ID NO: 113 and SEQ ID NO: 115; SEQ ID NO: 113 and SEQ ID NO: 116; SEQ ID NO: 113 and SEQ ID NO: 117; SEQ ID NO: 113 and SEQ ID NO: 118; SEQ ID NO: 113 and SEQ ID NO: 119; SEQ ID NO: 113 and SEQ ID NO: 120; SEQ ID NO: 113 and SEQ ID NO: 121; SEQ ID NO: 113 and SEQ ID NO: 122; SEQ ID NO: 113 and SEQ ID NO: 123; SEQ ID NO: 113 and SEQ ID NO: 124; SEQ ID NO: 113 and SEQ ID NO: 125; SEQ ID NO: 114 and SEQ ID NO: 115; SEQ ID NO: 114 and SEQ ID NO: 116; SEQ ID NO: 114 and SEQ ID NO: 117; SEQ ID NO: 114 and SEQ ID NO: 118; SEQ ID NO: 114 and SEQ ID NO: 119; SEQ ID NO: 114 and SEQ ID NO: 120; SEQ ID NO: 114 and SEQ ID NO: 121; SEQ ID NO: 114 and SEQ ID NO: 122; SEQ ID NO: 114 and SEQ ID NO: 123; SEQ ID NO: 114 and SEQ ID NO: 124; SEQ ID NO: 114 and SEQ ID NO: 125; SEQ ID NO: 115 and SEQ ID NO: 116; SEQ ID NO: 115 and SEQ ID NO: 117; SEQ ID NO: 115 and SEQ ID NO: 118; SEQ ID NO: 115 and SEQ ID NO: 119; SEQ ID NO: 115 and SEQ ID NO: 120; SEQ ID NO: 115 and SEQ ID NO: 121; SEQ ID NO: 115 and SEQ ID NO: 122; SEQ ID NO: 115 and SEQ ID NO: 123; SEQ ID NO: 115 and SEQ ID NO: 124; SEQ ID NO: 115 and SEQ ID NO: 125; SEQ ID NO: 116 and SEQ ID NO: 117; SEQ ID NO: 116 and SEQ ID NO: 118; SEQ ID NO: 116 and SEQ ID NO: 119; SEQ ID NO: 116 and SEQ ID NO: 120; SEQ ID NO: 116 and SEQ ID NO: 121; SEQ ID NO: 116 and SEQ ID NO: 122; SEQ ID NO: 116 and SEQ ID NO: 123; SEQ ID NO: 116 and SEQ ID NO: 124; SEQ ID NO: 116 and SEQ ID NO: 125; SEQ ID NO: 117 and SEQ ID NO: 118; SEQ ID NO: 117 and SEQ ID NO: 119; SEQ ID NO: 117 and SEQ ID NO: 120; SEQ ID NO: 117 and SEQ ID NO: 121; SEQ ID NO: 117 and SEQ ID NO: 122; SEQ ID NO: 117 and SEQ ID NO: 123; SEQ ID NO: 117 and SEQ ID NO: 124; SEQ ID NO: 117 and SEQ ID NO: 125; SEQ ID NO: 118 and SEQ ID NO: 119; SEQ ID NO: 118 and SEQ ID NO: 120; SEQ ID NO: 118 and SEQ ID NO: 121; SEQ ID NO: 118 and SEQ ID NO: 122; SEQ ID NO: 118 and SEQ ID NO: 123; SEQ ID NO: 118 and SEQ ID NO: 124; SEQ ID NO: 118 and SEQ ID NO: 125; SEQ ID NO: 119 and SEQ ID NO: 120; SEQ ID NO: 119 and SEQ ID NO: 121; SEQ ID NO: 119 and SEQ ID NO: 122; SEQ ID NO: 119 and SEQ ID NO: 123; SEQ ID NO: 119 and SEQ ID NO: 124; SEQ ID NO: 119 and SEQ ID NO: 125; SEQ ID NO: 120 and SEQ ID NO: 121; SEQ ID NO: 120 and SEQ ID NO: 122; SEQ ID NO: 120 and SEQ ID NO: 123; SEQ ID NO: 120 and SEQ ID NO: 124; SEQ ID NO: 120 and SEQ ID NO: 125; SEQ ID NO: 121 and SEQ ID NO: 122; SEQ ID NO: 121 and SEQ ID NO: 123; SEQ ID NO: 121 and SEQ ID NO: 124; SEQ ID NO: 121 and SEQ ID NO: 125; SEQ ID NO: 122 and SEQ ID NO: 123; SEQ ID NO: 122 and SEQ ID NO: 124; SEQ ID NO: 122 and SEQ ID NO: 125; SEQ ID NO: 123 and SEQ ID NO: 124; SEQ ID NO: 123 and SEQ ID NO: 125; SEQ ID NO: 124 and SEQ ID NO: 125; SEQ ID NO: 148 and SEQ ID NO: 149; SEQ ID NO: 148 and SEQ ID NO: 150; SEQ ID NO: 148 and SEQ ID NO: 151; SEQ ID NO: 148 and SEQ ID NO: 152; SEQ ID NO: 148 and SEQ ID NO: 153; SEQ ID NO: 148 and SEQ ID NO: 154; SEQ ID NO: 148 and SEQ ID NO: 155; SEQ ID NO: 148 and SEQ ID NO: 156; SEQ ID NO: 148 and SEQ ID NO: 157; SEQ ID NO: 148 and SEQ ID NO: 158; SEQ ID NO: 148 and SEQ ID NO: 159; SEQ ID NO: 149 and SEQ ID NO: 150; SEQ ID NO: 149 and SEQ ID NO: 151; SEQ ID NO: 149 and SEQ ID NO: 152; SEQ ID NO: 149 and SEQ ID NO: 153; SEQ ID NO: 149 and SEQ ID NO: 154; SEQ ID NO: 149 and SEQ ID NO: 155; SEQ ID NO: 149 and SEQ ID NO: 156; SEQ ID NO: 149 and SEQ ID NO: 157; SEQ ID NO: 149 and SEQ ID NO: 158; SEQ ID NO: 149 and SEQ ID NO: 159; SEQ ID NO: 150 and SEQ ID NO: 151; SEQ ID NO: 150 and SEQ ID NO: 152; SEQ ID NO: 150 and SEQ ID NO: 153; SEQ ID NO: 150 and SEQ ID NO: 154; SEQ ID NO: 150 and SEQ ID NO: 155; SEQ ID NO: 150 and SEQ ID NO: 156; SEQ ID NO: 150 and SEQ ID NO: 157; SEQ ID NO: 150 and SEQ ID NO: 158; SEQ ID NO: 150 and SEQ ID NO: 159; SEQ ID NO: 151 and SEQ ID NO: 152; SEQ ID NO: 151 and SEQ ID NO: 153; SEQ ID NO: 151 and SEQ ID NO: 154; SEQ ID NO: 151 and SEQ ID NO: 155; SEQ ID NO: 151 and SEQ ID NO: 156; SEQ ID NO: 151 and SEQ ID NO: 157; SEQ ID NO: 151 and SEQ ID NO: 158; SEQ ID NO: 151 and SEQ ID NO: 159; SEQ ID NO: 152 and SEQ ID NO: 153; SEQ ID NO: 152 and SEQ ID NO: 154; SEQ ID NO: 152 and SEQ ID NO: 155; SEQ ID NO: 152 and SEQ ID NO: 156; SEQ ID NO: 152 and SEQ ID NO: 157; SEQ ID NO: 152 and SEQ ID NO: 158; SEQ ID NO: 152 and SEQ ID NO: 159; SEQ ID NO: 153 and SEQ ID NO: 154; SEQ ID NO: 153 and SEQ ID NO: 155; SEQ ID NO: 153 and SEQ ID NO: 156; SEQ ID NO: 153 and SEQ ID NO: 157; SEQ ID NO: 153 and SEQ ID NO: 158; SEQ ID NO: 153 and SEQ ID NO: 159; SEQ ID NO: 154 and SEQ ID NO: 155; SEQ ID NO: 154 and SEQ ID NO: 156; SEQ ID NO: 154 and SEQ ID NO: 157; SEQ ID NO: 154 and SEQ ID NO: 158; SEQ ID NO: 154 and SEQ ID NO: 159; SEQ ID NO: 155 and SEQ ID NO: 156; SEQ ID NO: 155 and SEQ ID NO: 157; SEQ ID NO: 155 and SEQ ID NO: 158; SEQ ID NO: 155 and SEQ ID NO: 159; SEQ ID NO: 156 and SEQ ID NO: 157; SEQ ID NO: 156 and SEQ ID NO: 158; SEQ ID NO: 156 and SEQ ID NO: 159; SEQ ID NO: 157 and SEQ ID NO: 158; SEQ ID NO: 157 and SEQ ID NO: 159; SEQ ID NO: 158 and SEQ ID NO: 159; SEQ ID NO: 71 and SEQ ID NO: 72; SEQ ID NO: 71 and SEQ ID NO: 73; SEQ ID NO: 71 and SEQ ID NO: 74; SEQ ID NO: 71 and SEQ ID NO: 75; SEQ ID NO: 71 and SEQ ID NO: 76; SEQ ID NO: 71 and SEQ ID NO: 77; SEQ ID NO: 71 and SEQ ID NO: 78; SEQ ID NO: 71 and SEQ ID NO: 84; SEQ ID NO: 71 and SEQ ID NO: 85; SEQ ID NO: 71 and SEQ ID NO: 86; SEQ ID NO: 71 and SEQ ID NO: 87; SEQ ID NO: 71 and SEQ ID NO: 88; SEQ ID NO: 71 and SEQ ID NO: 89; SEQ ID NO: 71 and SEQ ID NO: 90; SEQ ID NO: 71 and SEQ ID NO: 91; SEQ ID NO: 71 and SEQ ID NO: 92; SEQ ID NO: 71 and SEQ ID NO: 93; SEQ ID NO: 71 and SEQ ID NO: 94; SEQ ID NO: 71 and SEQ ID NO: 95; SEQ ID NO: 71 and SEQ ID NO: 96; SEQ ID NO: 71 and SEQ ID NO: 97; SEQ ID NO: 71 and SEQ ID NO: 98; SEQ ID NO: 71 and SEQ ID NO: 99; SEQ ID NO: 71 and SEQ ID NO: 100; SEQ ID NO: 71 and SEQ ID NO: 101; SEQ ID NO: 71 and SEQ ID NO: 102; SEQ ID NO: 72 and SEQ ID NO: 73; SEQ ID NO: 72 and SEQ ID NO: 74; SEQ ID NO: 72 and SEQ ID NO: 75; SEQ ID NO: 72 and SEQ ID NO: 76; SEQ ID NO: 72 and SEQ ID NO: 77; SEQ ID NO: 72 and SEQ ID NO: 78; SEQ ID NO: 72 and SEQ ID NO: 84; SEQ ID NO: 72 and SEQ ID NO: 85; SEQ ID NO: 72 and SEQ ID NO: 86; SEQ ID NO: 72 and SEQ ID NO: 87; SEQ ID NO: 72 and SEQ ID NO: 88; SEQ ID NO: 72 and SEQ ID NO: 89; SEQ ID NO: 72 and SEQ ID NO: 90; SEQ ID NO: 72 and SEQ ID NO: 91; SEQ ID NO: 72 and SEQ ID NO: 92; SEQ ID NO: 72 and SEQ ID NO: 93; SEQ ID NO: 72 and SEQ ID NO: 94; SEQ ID NO: 72 and SEQ ID NO: 95; SEQ ID NO: 72 and SEQ ID NO: 96; SEQ ID NO: 72 and SEQ ID NO: 97; SEQ ID NO: 72 and SEQ ID NO: 98; SEQ ID NO: 72 and SEQ ID NO: 99; SEQ ID NO: 72 and SEQ ID NO: 100; SEQ ID NO: 72 and SEQ ID NO: 101; SEQ ID NO: 72 and SEQ ID NO: 102; SEQ ID NO: 73 and SEQ ID NO: 74; SEQ ID NO: 73 and SEQ ID NO: 75; SEQ ID NO: 73 and SEQ ID NO: 76; SEQ ID NO: 73 and SEQ ID NO: 77; SEQ ID NO: 73 and SEQ ID NO: 78; SEQ ID NO: 73 and SEQ ID NO: 84; SEQ ID NO: 73 and SEQ ID NO: 85; SEQ ID NO: 73 and SEQ ID NO: 86; SEQ ID NO: 73 and SEQ ID NO: 87; SEQ ID NO: 73 and SEQ ID NO: 88; SEQ ID NO: 73 and SEQ ID NO: 89; SEQ ID NO: 73 and SEQ ID NO: 90; SEQ ID NO: 73 and SEQ ID NO: 91; SEQ ID NO: 73 and SEQ ID NO: 92; SEQ ID NO: 73 and SEQ ID NO: 93; SEQ ID NO: 73 and SEQ ID NO: 94; SEQ ID NO: 73 and SEQ ID NO: 95; SEQ ID NO: 73 and SEQ ID NO: 96; SEQ ID NO: 73 and SEQ ID NO: 97; SEQ ID NO: 73 and SEQ ID NO: 98; SEQ ID NO: 73 and SEQ ID NO: 99; SEQ ID NO: 73 and SEQ ID NO: 100; SEQ ID NO: 73 and SEQ ID NO: 101; SEQ ID NO: 73 and SEQ ID NO: 102; SEQ ID NO: 74 and SEQ ID NO: 75; SEQ ID NO: 74 and SEQ ID NO: 76; SEQ ID NO: 74 and SEQ ID NO: 77; SEQ ID NO: 74 and SEQ ID NO: 78; SEQ ID NO: 74 and SEQ ID NO: 84; SEQ ID NO: 74 and SEQ ID NO: 85; SEQ ID NO: 74 and SEQ ID NO: 86; SEQ ID NO: 74 and SEQ ID NO: 87; SEQ ID NO: 74 and SEQ ID NO: 88; SEQ ID NO: 74 and SEQ ID NO: 89; SEQ ID NO: 74 and SEQ ID NO: 90; SEQ ID NO: 74 and SEQ ID NO: 91; SEQ ID NO: 74 and SEQ ID NO: 92; SEQ ID NO: 74 and SEQ ID NO: 93; SEQ ID NO: 74 and SEQ ID NO: 94; SEQ ID NO: 74 and SEQ ID NO: 95; SEQ ID NO: 74 and SEQ ID NO: 96; SEQ ID NO: 74 and SEQ ID NO: 97; SEQ ID NO: 74 and SEQ ID NO: 98; SEQ ID NO: 74 and SEQ ID NO: 99; SEQ ID NO: 74 and SEQ ID NO: 100; SEQ ID NO: 74 and SEQ ID NO: 101; SEQ ID NO: 74 and SEQ ID NO: 102; SEQ ID NO: 75 and SEQ ID NO: 76; SEQ ID NO: 75 and SEQ ID NO: 77; SEQ ID NO: 75 and SEQ ID NO: 78; SEQ ID NO: 75 and SEQ ID NO: 84; SEQ ID NO: 75 and SEQ ID NO: 85; SEQ ID NO: 75 and SEQ ID NO: 86; SEQ ID NO: 75 and SEQ ID NO: 87; SEQ ID NO: 75 and SEQ ID NO: 88; SEQ ID NO: 75 and SEQ ID NO: 89; SEQ ID NO: 75 and SEQ ID NO: 90; SEQ ID NO: 75 and SEQ ID NO: 91; SEQ ID NO: 75 and SEQ ID NO: 92; SEQ ID NO: 75 and SEQ ID NO: 93; SEQ ID NO: 75 and SEQ ID NO: 94; SEQ ID NO: 75 and SEQ ID NO: 95; SEQ ID NO: 75 and SEQ ID NO: 96; SEQ ID NO: 75 and SEQ ID NO: 97; SEQ ID NO: 75 and SEQ ID NO: 98; SEQ ID NO: 75 and SEQ ID NO: 99; SEQ ID NO: 75 and SEQ ID NO: 100; SEQ ID NO: 75 and SEQ ID NO: 101; SEQ ID NO: 75 and SEQ ID NO: 102; SEQ ID NO: 76 and SEQ ID NO: 77; SEQ ID NO: 76 and SEQ ID NO: 78; SEQ ID NO: 76 and SEQ ID NO: 84; SEQ ID NO: 76 and SEQ ID NO: 85; SEQ ID NO: 76 and SEQ ID NO: 86; SEQ ID NO: 76 and SEQ ID NO: 87; SEQ ID NO: 76 and SEQ ID NO: 88; SEQ ID NO: 76 and SEQ ID NO: 89; SEQ ID NO: 76 and SEQ ID NO: 90; SEQ ID NO: 76 and SEQ ID NO: 91; SEQ ID NO: 76 and SEQ ID NO: 92; SEQ ID NO: 76 and SEQ ID NO: 93; SEQ ID NO: 76 and SEQ ID NO: 94; SEQ ID NO: 76 and SEQ ID NO: 95; SEQ ID NO: 76 and SEQ ID NO: 96; SEQ ID NO: 76 and SEQ ID NO: 97; SEQ ID NO: 76 and SEQ ID NO: 98; SEQ ID NO: 76 and SEQ ID NO: 99; SEQ ID NO: 76 and SEQ ID NO: 100; SEQ ID NO: 76 and SEQ ID NO: 101; SEQ ID NO: 76 and SEQ ID NO: 102; SEQ ID NO: 77 and SEQ ID NO: 78; SEQ ID NO: 77 and SEQ ID NO: 84; SEQ ID NO: 77 and SEQ ID NO: 85; SEQ ID NO: 77 and SEQ ID NO: 86; SEQ ID NO: 77 and SEQ ID NO: 87; SEQ ID NO: 77 and SEQ ID NO: 88; SEQ ID NO: 77 and SEQ ID NO: 89; SEQ ID NO: 77 and SEQ ID NO: 90; SEQ ID NO: 77 and SEQ ID NO: 91; SEQ ID NO: 77 and SEQ ID NO: 92; SEQ ID NO: 77 and SEQ ID NO: 93; SEQ ID NO: 77 and SEQ ID NO: 94; SEQ ID NO: 77 and SEQ ID NO: 95; SEQ ID NO: 77 and SEQ ID NO: 96; SEQ ID NO: 77 and SEQ ID NO: 97; SEQ ID NO: 77 and SEQ ID NO: 98; SEQ ID NO: 77 and SEQ ID NO: 99; SEQ ID NO: 77 and SEQ ID NO: 100; SEQ ID NO: 77 and SEQ ID NO: 101; SEQ ID NO: 77 and SEQ ID NO: 102; SEQ ID NO: 78 and SEQ ID NO: 84; SEQ ID NO: 78 and SEQ ID NO: 85; SEQ ID NO: 78 and SEQ ID NO: 86; SEQ ID NO: 78 and SEQ ID NO: 87; SEQ ID NO: 78 and SEQ ID NO: 88; SEQ ID NO: 78 and SEQ ID NO: 89; SEQ ID NO: 78 and SEQ ID NO: 90; SEQ ID NO: 78 and SEQ ID NO: 91; SEQ ID NO: 78 and SEQ ID NO: 92; SEQ ID NO: 78 and SEQ ID NO: 93; SEQ ID NO: 78 and SEQ ID NO: 94; SEQ ID NO: 78 and SEQ ID NO: 95; SEQ ID NO: 78 and SEQ ID NO: 96; SEQ ID NO: 78 and SEQ ID NO: 97; SEQ ID NO: 78 and SEQ ID NO: 98; SEQ ID NO: 78 and SEQ ID NO: 99; SEQ ID NO: 78 and SEQ ID NO: 100; SEQ ID NO: 78 and SEQ ID NO: 101; SEQ ID NO: 78 and SEQ ID NO: 102; SEQ ID NO: 84 and SEQ ID NO: 85; SEQ ID NO: 84 and SEQ ID NO: 86; SEQ ID NO: 84 and SEQ ID NO: 87; SEQ ID NO: 84 and SEQ ID NO: 88; SEQ ID NO: 84 and SEQ ID NO: 89; SEQ ID NO: 84 and SEQ ID NO: 90; SEQ ID NO: 84 and SEQ ID NO: 91; SEQ ID NO: 84 and SEQ ID NO: 92; SEQ ID NO: 84 and SEQ ID NO: 93; SEQ ID NO: 84 and SEQ ID NO: 94; SEQ ID NO: 84 and SEQ ID NO: 95; SEQ ID NO: 84 and SEQ ID NO: 96; SEQ ID NO: 84 and SEQ ID NO: 97; SEQ ID NO: 84 and SEQ ID NO: 98; SEQ ID NO: 84 and SEQ ID NO: 99; SEQ ID NO: 84 and SEQ ID NO: 100; SEQ ID NO: 84 and SEQ ID NO: 101; SEQ ID NO: 84 and SEQ ID NO: 102; SEQ ID NO: 85 and SEQ ID NO: 86; SEQ ID NO: 85 and SEQ ID NO: 87; SEQ ID NO: 85 and SEQ ID NO: 88; SEQ ID NO: 85 and SEQ ID NO: 89; SEQ ID NO: 85 and SEQ ID NO: 90; SEQ ID NO: 85 and SEQ ID NO: 91; SEQ ID NO: 85 and SEQ ID NO: 92; SEQ ID NO: 85 and SEQ ID NO: 93; SEQ ID NO: 85 and SEQ ID NO: 94; SEQ ID NO: 85 and SEQ ID NO: 95; SEQ ID NO: 85 and SEQ ID NO: 96; SEQ ID NO: 85 and SEQ ID NO: 97; SEQ ID NO: 85 and SEQ ID NO: 98; SEQ ID NO: 85 and SEQ ID NO: 99; SEQ ID NO: 85 and SEQ ID NO: 100; SEQ ID NO: 85 and SEQ ID NO: 101; SEQ ID NO: 85 and SEQ ID NO: 102; SEQ ID NO: 86 and SEQ ID NO: 87; SEQ ID NO: 86 and SEQ ID NO: 88; SEQ ID NO: 86 and SEQ ID NO: 89; SEQ ID NO: 86 and SEQ ID NO: 90; SEQ ID NO: 86 and SEQ ID NO: 91; SEQ ID NO: 86 and SEQ ID NO: 92; SEQ ID NO: 86 and SEQ ID NO: 93; SEQ ID NO: 86 and SEQ ID NO: 94; SEQ ID NO: 86 and SEQ ID NO: 95; SEQ ID NO: 86 and SEQ ID NO: 96; SEQ ID NO: 86 and SEQ ID NO: 97; SEQ ID NO: 86 and SEQ ID NO: 98; SEQ ID NO: 86 and SEQ ID NO: 99; SEQ ID NO: 86 and SEQ ID NO: 100; SEQ ID NO: 86 and SEQ ID NO: 101; SEQ ID NO: 86 and SEQ ID NO: 102; SEQ ID NO: 87 and SEQ ID NO: 88; SEQ ID NO: 87 and SEQ ID NO: 89; SEQ ID NO: 87 and SEQ ID NO: 90; SEQ ID NO: 87 and SEQ ID NO: 91; SEQ ID NO: 87 and SEQ ID NO: 92; SEQ ID NO: 87 and SEQ ID NO: 93; SEQ ID NO: 87 and SEQ ID NO: 94; SEQ ID NO: 87 and SEQ ID NO: 95; SEQ ID NO: 87 and SEQ ID NO: 96; SEQ ID NO: 87 and SEQ ID NO: 97; SEQ ID NO: 87 and SEQ ID NO: 98; SEQ ID NO: 87 and SEQ ID NO: 99; SEQ ID NO: 87 and SEQ ID NO: 100; SEQ ID NO: 87 and SEQ ID NO: 101; SEQ ID NO: 87 and SEQ ID NO: 102; SEQ ID NO: 88 and SEQ ID NO: 89; SEQ ID NO: 88 and SEQ ID NO: 90; SEQ ID NO: 88 and SEQ ID NO: 91; SEQ ID NO: 88 and SEQ ID NO: 92; SEQ ID NO: 88 and SEQ ID NO: 93; SEQ ID NO: 88 and SEQ ID NO: 94; SEQ ID NO: 88 and SEQ ID NO: 95; SEQ ID NO: 88 and SEQ ID NO: 96; SEQ ID NO: 88 and SEQ ID NO: 97; SEQ ID NO: 88 and SEQ ID NO: 98; SEQ ID NO: 88 and SEQ ID NO: 99; SEQ ID NO: 88 and SEQ ID NO: 100; SEQ ID NO: 88 and SEQ ID NO: 101; SEQ ID NO: 88 and SEQ ID NO: 102; SEQ ID NO: 89 and SEQ ID NO: 90; SEQ ID NO: 89 and SEQ ID NO: 91; SEQ ID NO: 89 and SEQ ID NO: 92; SEQ ID NO: 89 and SEQ ID NO: 93; SEQ ID NO: 89 and SEQ ID NO: 94; SEQ ID NO: 89 and SEQ ID NO: 95; SEQ ID NO: 89 and SEQ ID NO: 96; SEQ ID NO: 89 and SEQ ID NO: 97; SEQ ID NO: 89 and SEQ ID NO: 98; SEQ ID NO: 89 and SEQ ID NO: 99; SEQ ID NO: 89 and SEQ ID NO: 100; SEQ ID NO: 89 and SEQ ID NO: 101; SEQ ID NO: 89 and SEQ ID NO: 102; SEQ ID NO: 90 and SEQ ID NO: 91; SEQ ID NO: 90 and SEQ ID NO: 92; SEQ ID NO: 90 and SEQ ID NO: 93; SEQ ID NO: 90 and SEQ ID NO: 94; SEQ ID NO: 90 and SEQ ID NO: 95; SEQ ID NO: 90 and SEQ ID NO: 96; SEQ ID NO: 90 and SEQ ID NO: 97; SEQ ID NO: 90 and SEQ ID NO: 98; SEQ ID NO: 90 and SEQ ID NO: 99; SEQ ID NO: 90 and SEQ ID NO: 100; SEQ ID NO: 90 and SEQ ID NO: 101; SEQ ID NO: 90 and SEQ ID NO: 102; SEQ ID NO: 91 and SEQ ID NO: 92; SEQ ID NO: 91 and SEQ ID NO: 93; SEQ ID NO: 91 and SEQ ID NO: 94; SEQ ID NO: 91 and SEQ ID NO: 95; SEQ ID NO: 91 and SEQ ID NO: 96; SEQ ID NO: 91 and SEQ ID NO: 97; SEQ ID NO: 91 and SEQ ID NO: 98; SEQ ID NO: 91 and SEQ ID NO: 99; SEQ ID NO: 91 and SEQ ID NO: 100; SEQ ID NO: 91 and SEQ ID NO: 101; SEQ ID NO: 91 and SEQ ID NO: 102; SEQ ID NO: 92 and SEQ ID NO: 93; SEQ ID NO: 92 and SEQ ID NO: 94; SEQ ID NO: 92 and SEQ ID NO: 95; SEQ ID NO: 92 and SEQ ID NO: 96; SEQ ID NO: 92 and SEQ ID NO: 97; SEQ ID NO: 92 and SEQ ID NO: 98; SEQ ID NO: 92 and SEQ ID NO: 99; SEQ ID NO: 92 and SEQ ID NO: 100; SEQ ID NO: 92 and SEQ ID NO: 101; SEQ ID NO: 92 and SEQ ID NO: 102; SEQ ID NO: 93 and SEQ ID NO: 94; SEQ ID NO: 93 and SEQ ID NO: 95; SEQ ID NO: 93 and SEQ ID NO: 96; SEQ ID NO: 93 and SEQ ID NO: 97; SEQ ID NO: 93 and SEQ ID NO: 98; SEQ ID NO: 93 and SEQ ID NO: 99; SEQ ID NO: 93 and SEQ ID NO: 100; SEQ ID NO: 93 and SEQ ID NO: 101; SEQ ID NO: 93 and SEQ ID NO: 102; SEQ ID NO: 94 and SEQ ID NO: 95; SEQ ID NO: 94 and SEQ ID NO: 96; SEQ ID NO: 94 and SEQ ID NO: 97; SEQ ID NO: 94 and SEQ ID NO: 98; SEQ ID NO: 94 and SEQ ID NO: 99; SEQ ID NO: 94 and SEQ ID NO: 100; SEQ ID NO: 94 and SEQ ID NO: 101; SEQ ID NO: 94 and SEQ ID NO: 102; SEQ ID NO: 95 and SEQ ID NO: 96; SEQ ID NO: 95 and SEQ ID NO: 97; SEQ ID NO: 95 and SEQ ID NO: 98; SEQ ID NO: 95 and SEQ ID NO: 99; SEQ ID NO: 95 and SEQ ID NO: 100; SEQ ID NO: 95 and SEQ ID NO: 101; SEQ ID NO: 95 and SEQ ID NO: 102; SEQ ID NO: 96 and SEQ ID NO: 97; SEQ ID NO: 96 and SEQ ID NO: 98; SEQ ID NO: 96 and SEQ ID NO: 99; SEQ ID NO: 96 and SEQ ID NO: 100; SEQ ID NO: 96 and SEQ ID NO: 101; SEQ ID NO: 96 and SEQ ID NO: 102; SEQ ID NO: 97 and SEQ ID NO: 98; SEQ ID NO: 97 and SEQ ID NO: 99; SEQ ID NO: 97 and SEQ ID NO: 100; SEQ ID NO: 97 and SEQ ID NO: 101; SEQ ID NO: 97 and SEQ ID NO: 102; SEQ ID NO: 98 and SEQ ID NO: 99; SEQ ID NO: 98 and SEQ ID NO: 100; SEQ ID NO: 98 and SEQ ID NO: 101; SEQ ID NO: 98 and SEQ ID NO: 102; SEQ ID NO: 99 and SEQ ID NO: 100; SEQ ID NO: 99 and SEQ ID NO: 101; SEQ ID NO: 99 and SEQ ID NO: 102; SEQ ID NO: 100 and SEQ ID NO: 101; SEQ ID NO: 100 and SEQ ID NO: 102; SEQ ID NO: 101 and SEQ ID NO: 102; SEQ ID NO: 200 and SEQ ID NO: 201; SEQ ID NO: 200 and SEQ ID NO: 202; SEQ ID NO: 200 and SEQ ID NO: 203; SEQ ID NO: 200 and SEQ ID NO: 204; SEQ ID NO: 200 and SEQ ID NO: 205; SEQ ID NO: 200 and SEQ ID NO: 206; SEQ ID NO: 200 and SEQ ID NO: 207; SEQ ID NO: 200 and SEQ ID NO: 208; SEQ ID NO: 201 and SEQ ID NO: 202; SEQ ID NO: 201 and SEQ ID NO: 203; SEQ ID NO: 201 and SEQ ID NO: 204; SEQ ID NO: 201 and SEQ ID NO: 205; SEQ ID NO: 201 and SEQ ID NO: 206; SEQ ID NO: 201 and SEQ ID NO: 207; SEQ ID NO: 201 and SEQ ID NO: 208; SEQ ID NO: 202 and SEQ ID NO: 203; SEQ ID NO: 202 and SEQ ID NO: 204; SEQ ID NO: 202 and SEQ ID NO: 205; SEQ ID NO: 202 and SEQ ID NO: 206; SEQ ID NO: 202 and SEQ ID NO: 207; SEQ ID NO: 202 and SEQ ID NO: 208; SEQ ID NO: 203 and SEQ ID NO: 204; SEQ ID NO: 203 and SEQ ID NO: 205; SEQ ID NO: 203 and SEQ ID NO: 206; SEQ ID NO: 203 and SEQ ID NO: 207; SEQ ID NO: 203 and SEQ ID NO: 208; SEQ ID NO: 204 and SEQ ID NO: 205; SEQ ID NO: 204 and SEQ ID NO: 206; SEQ ID NO: 204 and SEQ ID NO: 207; SEQ ID NO: 204 and SEQ ID NO: 208; SEQ ID NO: 205 and SEQ ID NO: 206; SEQ ID NO: 205 and SEQ ID NO: 207; SEQ ID NO: 205 and SEQ ID NO: 208; SEQ ID NO: 206 and SEQ ID NO: 207; SEQ ID NO: 206 and SEQ ID NO: 208; SEQ ID NO: 207 and SEQ ID NO: 208; SEQ ID NO: 231 and SEQ ID NO: 232; SEQ ID NO: 231 and SEQ ID NO: 233; SEQ ID NO: 231 and SEQ ID NO: 234; SEQ ID NO: 231 and SEQ ID NO: 235; SEQ ID NO: 231 and SEQ ID NO: 236; SEQ ID NO: 231 and SEQ ID NO: 237; SEQ ID NO: 231 and SEQ ID NO: 238; SEQ ID NO: 231 and SEQ ID NO: 239; SEQ ID NO: 231 and SEQ ID NO: 240; SEQ ID NO: 231 and SEQ ID NO: 241; SEQ ID NO: 231 and SEQ ID NO: 242; SEQ ID NO: 232 and SEQ ID NO: 233; SEQ ID NO: 232 and SEQ ID NO: 234; SEQ ID NO: 232 and SEQ ID NO: 235; SEQ ID NO: 232 and SEQ ID NO: 236; SEQ ID NO: 232 and SEQ ID NO: 237; SEQ ID NO: 232 and SEQ ID NO: 238; SEQ ID NO: 232 and SEQ ID NO: 239; SEQ ID NO: 232 and SEQ ID NO: 240; SEQ ID NO: 232 and SEQ ID NO: 241; SEQ ID NO: 232 and SEQ ID NO: 242; SEQ ID NO: 233 and SEQ ID NO: 234; SEQ ID NO: 233 and SEQ ID NO: 235; SEQ ID NO: 233 and SEQ ID NO: 236; SEQ ID NO: 233 and SEQ ID NO: 237; SEQ ID NO: 233 and SEQ ID NO: 238; SEQ ID NO: 233 and SEQ ID NO: 239; SEQ ID NO: 233 and SEQ ID NO: 240; SEQ ID NO: 233 and SEQ ID NO: 241; SEQ ID NO: 233 and SEQ ID NO: 242; SEQ ID NO: 234 and SEQ ID NO: 235; SEQ ID NO: 234 and SEQ ID NO: 236; SEQ ID NO: 234 and SEQ ID NO: 237; SEQ ID NO: 234 and SEQ ID NO: 238; SEQ ID NO: 234 and SEQ ID NO: 239; SEQ ID NO: 234 and SEQ ID NO: 240; SEQ ID NO: 234 and SEQ ID NO: 241; SEQ ID NO: 234 and SEQ ID NO: 242; SEQ ID NO: 235 and SEQ ID NO: 236; SEQ ID NO: 235 and SEQ ID NO: 237; SEQ ID NO: 235 and SEQ ID NO: 238; SEQ ID NO: 235 and SEQ ID NO: 239; SEQ ID NO: 235 and SEQ ID NO: 240; SEQ ID NO: 235 and SEQ ID NO: 241; SEQ ID NO: 235 and SEQ ID NO: 242; SEQ ID NO: 236 and SEQ ID NO: 237; SEQ ID NO: 236 and SEQ ID NO: 238; SEQ ID NO: 236 and SEQ ID NO: 239; SEQ ID NO: 236 and SEQ ID NO: 240; SEQ ID NO: 236 and SEQ ID NO: 241; SEQ ID NO: 236 and SEQ ID NO: 242; SEQ ID NO: 237 and SEQ ID NO: 238; SEQ ID NO: 237 and SEQ ID NO: 239; SEQ ID NO: 237 and SEQ ID NO: 240; SEQ ID NO: 237 and SEQ ID NO: 241; SEQ ID NO: 237 and SEQ ID NO: 242; SEQ ID NO: 238 and SEQ ID NO: 239; SEQ ID NO: 238 and SEQ ID NO: 240; SEQ ID NO: 238 and SEQ ID NO: 241; SEQ ID NO: 238 and SEQ ID NO: 242; SEQ ID NO: 239 and SEQ ID NO: 240; SEQ ID NO: 239 and SEQ ID NO: 241; SEQ ID NO: 239 and SEQ ID NO: 242; SEQ ID NO: 240 and SEQ ID NO: 241; SEQ ID NO: 240 and SEQ ID NO: 242; SEQ ID NO: 241 and SEQ ID NO: 242; SEQ ID NO: 270 and SEQ ID NO: 271; SEQ ID NO: 270 and SEQ ID NO: 272; SEQ ID NO: 270 and SEQ ID NO: 273; SEQ ID NO: 270 and SEQ ID NO: 274; SEQ ID NO: 270 and SEQ ID NO: 275; SEQ ID NO: 270 and SEQ ID NO: 276; SEQ ID NO: 270 and SEQ ID NO: 277; SEQ ID NO: 270 and SEQ ID NO: 278; SEQ ID NO: 270 and SEQ ID NO: 279; SEQ ID NO: 270 and SEQ ID NO: 280; SEQ ID NO: 270 and SEQ ID NO: 281; SEQ ID NO: 270 and SEQ ID NO: 282; SEQ ID NO: 270 and SEQ ID NO: 283; SEQ ID NO: 270 and SEQ ID NO: 284; SEQ ID NO: 270 and SEQ ID NO: 285; SEQ ID NO: 270 and SEQ ID NO: 286; SEQ ID NO: 270 and SEQ ID NO: 287; SEQ ID NO: 270 and SEQ ID NO: 288; SEQ ID NO: 270 and SEQ ID NO: 289; SEQ ID NO: 270 and SEQ ID NO: 290; SEQ ID NO: 270 and SEQ ID NO: 291; SEQ ID NO: 270 and SEQ ID NO: 292; SEQ ID NO: 271 and SEQ ID NO: 272; SEQ ID NO: 271 and SEQ ID NO: 273; SEQ ID NO: 271 and SEQ ID NO: 274; SEQ ID NO: 271 and SEQ ID NO: 275; SEQ ID NO: 271 and SEQ ID NO: 276; SEQ ID NO: 271 and SEQ ID NO: 277; SEQ ID NO: 271 and SEQ ID NO: 278; SEQ ID NO: 271 and SEQ ID NO: 279; SEQ ID NO: 271 and SEQ ID NO: 280; SEQ ID NO: 271 and SEQ ID NO: 281; SEQ ID NO: 271 and SEQ ID NO: 282; SEQ ID NO: 271 and SEQ ID NO: 283; SEQ ID NO: 271 and SEQ ID NO: 284; SEQ ID NO: 271 and SEQ ID NO: 285; SEQ ID NO: 271 and SEQ ID NO: 286; SEQ ID NO: 271 and SEQ ID NO: 287; SEQ ID NO: 271 and SEQ ID NO: 288; SEQ ID NO: 271 and SEQ ID NO: 289; SEQ ID NO: 271 and SEQ ID NO: 290; SEQ ID NO: 271 and SEQ ID NO: 291; SEQ ID NO: 271 and SEQ ID NO: 292; SEQ ID NO: 272 and SEQ ID NO: 273; SEQ ID NO: 272 and SEQ ID NO: 274; SEQ ID NO: 272 and SEQ ID NO: 275; SEQ ID NO: 272 and SEQ ID NO: 276; SEQ ID NO: 272 and SEQ ID NO: 277; SEQ ID NO: 272 and SEQ ID NO: 278; SEQ ID NO: 272 and SEQ ID NO: 279; SEQ ID NO: 272 and SEQ ID NO: 280; SEQ ID NO: 272 and SEQ ID NO: 281; SEQ ID NO: 272 and SEQ ID NO: 282; SEQ ID NO: 272 and SEQ ID NO: 283; SEQ ID NO: 272 and SEQ ID NO: 284; SEQ ID NO: 272 and SEQ ID NO: 285; SEQ ID NO: 272 and SEQ ID NO: 286; SEQ ID NO: 272 and SEQ ID NO: 287; SEQ ID NO: 272 and SEQ ID NO: 288; SEQ ID NO: 272 and SEQ ID NO: 289; SEQ ID NO: 272 and SEQ ID NO: 290; SEQ ID NO: 272 and SEQ ID NO: 291; SEQ ID NO: 272 and SEQ ID NO: 292; SEQ ID NO: 273 and SEQ ID NO: 274; SEQ ID NO: 273 and SEQ ID NO: 275; SEQ ID NO: 273 and SEQ ID NO: 276; SEQ ID NO: 273 and SEQ ID NO: 277; SEQ ID NO: 273 and SEQ ID NO: 278; SEQ ID NO: 273 and SEQ ID NO: 279; SEQ ID NO: 273 and SEQ ID NO: 280; SEQ ID NO: 273 and SEQ ID NO: 281; SEQ ID NO: 273 and SEQ ID NO: 282; SEQ ID NO: 273 and SEQ ID NO: 283; SEQ ID NO: 273 and SEQ ID NO: 284; SEQ ID NO: 273 and SEQ ID NO: 285; SEQ ID NO: 273 and SEQ ID NO: 286; SEQ ID NO: 273 and SEQ ID NO: 287; SEQ ID NO: 273 and SEQ ID NO: 288; SEQ ID NO: 273 and SEQ ID NO: 289; SEQ ID NO: 273 and SEQ ID NO: 290; SEQ ID NO: 273 and SEQ ID NO: 291; SEQ ID NO: 273 and SEQ ID NO: 292; SEQ ID NO: 274 and SEQ ID NO: 275; SEQ ID NO: 274 and SEQ ID NO: 276; SEQ ID NO: 274 and SEQ ID NO: 277; SEQ ID NO: 274 and SEQ ID NO: 278; SEQ ID NO: 274 and SEQ ID NO: 279; SEQ ID NO: 274 and SEQ ID NO: 280; SEQ ID NO: 274 and SEQ ID NO: 281; SEQ ID NO: 274 and SEQ ID NO: 282; SEQ ID NO: 274 and SEQ ID NO: 283; SEQ ID NO: 274 and SEQ ID NO: 284; SEQ ID NO: 274 and SEQ ID NO: 285; SEQ ID NO: 274 and SEQ ID NO: 286; SEQ ID NO: 274 and SEQ ID NO: 287; SEQ ID NO: 274 and SEQ ID NO: 288; SEQ ID NO: 274 and SEQ ID NO: 289; SEQ ID NO: 274 and SEQ ID NO: 290; SEQ ID NO: 274 and SEQ ID NO: 291; SEQ ID NO: 274 and SEQ ID NO: 292; SEQ ID NO: 275 and SEQ ID NO: 276; SEQ ID NO: 275 and SEQ ID NO: 277; SEQ ID NO: 275 and SEQ ID NO: 278; SEQ ID NO: 275 and SEQ ID NO: 279; SEQ ID NO: 275 and SEQ ID NO: 280; SEQ ID NO: 275 and SEQ ID NO: 281; SEQ ID NO: 275 and SEQ ID NO: 282; SEQ ID NO: 275 and SEQ ID NO: 283; SEQ ID NO: 275 and SEQ ID NO: 284; SEQ ID NO: 275 and SEQ ID NO: 285; SEQ ID NO: 275 and SEQ ID NO: 286; SEQ ID NO: 275 and SEQ ID NO: 287; SEQ ID NO: 275 and SEQ ID NO: 288; SEQ ID NO: 275 and SEQ ID NO: 289; SEQ ID NO: 275 and SEQ ID NO: 290; SEQ ID NO: 275 and SEQ ID NO: 291; SEQ ID NO: 275 and SEQ ID NO: 292; SEQ ID NO: 276 and SEQ ID NO: 277; SEQ ID NO: 276 and SEQ ID NO: 278; SEQ ID NO: 276 and SEQ ID NO: 279; SEQ ID NO: 276 and SEQ ID NO: 280; SEQ ID NO: 276 and SEQ ID NO: 281; SEQ ID NO: 276 and SEQ ID NO: 282; SEQ ID NO: 276 and SEQ ID NO: 283; SEQ ID NO: 276 and SEQ ID NO: 284; SEQ ID NO: 276 and SEQ ID NO: 285; SEQ ID NO: 276 and SEQ ID NO: 286; SEQ ID NO: 276 and SEQ ID NO: 287; SEQ ID NO: 276 and SEQ ID NO: 288; SEQ ID NO: 276 and SEQ ID NO: 289; SEQ ID NO: 276 and SEQ ID NO: 290; SEQ ID NO: 276 and SEQ ID NO: 291; SEQ ID NO: 276 and SEQ ID NO: 292; SEQ ID NO: 277 and SEQ ID NO: 278; SEQ ID NO: 277 and SEQ ID NO: 279; SEQ ID NO: 277 and SEQ ID NO: 280; SEQ ID NO: 277 and SEQ ID NO: 281; SEQ ID NO: 277 and SEQ ID NO: 282; SEQ ID NO: 277 and SEQ ID NO: 283; SEQ ID NO: 277 and SEQ ID NO: 284; SEQ ID NO: 277 and SEQ ID NO: 285; SEQ ID NO: 277 and SEQ ID NO: 286; SEQ ID NO: 277 and SEQ ID NO: 287; SEQ ID NO: 277 and SEQ ID NO: 288; SEQ ID NO: 277 and SEQ ID NO: 289; SEQ ID NO: 277 and SEQ ID NO: 290; SEQ ID NO: 277 and SEQ ID NO: 291; SEQ ID NO: 277 and SEQ ID NO: 292; SEQ ID NO: 278 and SEQ ID NO: 279; SEQ ID NO: 278 and SEQ ID NO: 280; SEQ ID NO: 278 and SEQ ID NO: 281; SEQ ID NO: 278 and SEQ ID NO: 282; SEQ ID NO: 278 and SEQ ID NO: 283; SEQ ID NO: 278 and SEQ ID NO: 284; SEQ ID NO: 278 and SEQ ID NO: 285; SEQ ID NO: 278 and SEQ ID NO: 286; SEQ ID NO: 278 and SEQ ID NO: 287; SEQ ID NO: 278 and SEQ ID NO: 288; SEQ ID NO: 278 and SEQ ID NO: 289; SEQ ID NO: 278 and SEQ ID NO: 290; SEQ ID NO: 278 and SEQ ID NO: 291; SEQ ID NO: 278 and SEQ ID NO: 292; SEQ ID NO: 279 and SEQ ID NO: 280; SEQ ID NO: 279 and SEQ ID NO: 281; SEQ ID NO: 279 and SEQ ID NO: 282; SEQ ID NO: 279 and SEQ ID NO: 283; SEQ ID NO: 279 and SEQ ID NO: 284; SEQ ID NO: 279 and SEQ ID NO: 285; SEQ ID NO: 279 and SEQ ID NO: 286; SEQ ID NO: 279 and SEQ ID NO: 287; SEQ ID NO: 279 and SEQ ID NO: 288; SEQ ID NO: 279 and SEQ ID NO: 289; SEQ ID NO: 279 and SEQ ID NO: 290; SEQ ID NO: 279 and SEQ ID NO: 291; SEQ ID NO: 279 and SEQ ID NO: 292; SEQ ID NO: 280 and SEQ ID NO: 281; SEQ ID NO: 280 and SEQ ID NO: 282; SEQ ID NO: 280 and SEQ ID NO: 283; SEQ ID NO: 280 and SEQ ID NO: 284; SEQ ID NO: 280 and SEQ ID NO: 285; SEQ ID NO: 280 and SEQ ID NO: 286; SEQ ID NO: 280 and SEQ ID NO: 287; SEQ ID NO: 280 and SEQ ID NO: 288; SEQ ID NO: 280 and SEQ ID NO: 289; SEQ ID NO: 280 and SEQ ID NO: 290; SEQ ID NO: 280 and SEQ ID NO: 291; SEQ ID NO: 280 and SEQ ID NO: 292; SEQ ID NO: 281 and SEQ ID NO: 282; SEQ ID NO: 281 and SEQ ID NO: 283; SEQ ID NO: 281 and SEQ ID NO: 284; SEQ ID NO: 281 and SEQ ID NO: 285; SEQ ID NO: 281 and SEQ ID NO: 286; SEQ ID NO: 281 and SEQ ID NO: 287; SEQ ID NO: 281 and SEQ ID NO: 288; SEQ ID NO: 281 and SEQ ID NO: 289; SEQ ID NO: 281 and SEQ ID NO: 290; SEQ ID NO: 281 and SEQ ID NO: 291; SEQ ID NO: 281 and SEQ ID NO: 292; SEQ ID NO: 282 and SEQ ID NO: 283; SEQ ID NO: 282 and SEQ ID NO: 284; SEQ ID NO: 282 and SEQ ID NO: 285; SEQ ID NO: 282 and SEQ ID NO: 286; SEQ ID NO: 282 and SEQ ID NO: 287; SEQ ID NO: 282 and SEQ ID NO: 288; SEQ ID NO: 282 and SEQ ID NO: 289; SEQ ID NO: 282 and SEQ ID NO: 290; SEQ ID NO: 282 and SEQ ID NO: 291; SEQ ID NO: 282 and SEQ ID NO: 292; SEQ ID NO: 283 and SEQ ID NO: 284; SEQ ID NO: 283 and SEQ ID NO: 285; SEQ ID NO: 283 and SEQ ID NO: 286; SEQ ID NO: 283 and SEQ ID NO: 287; SEQ ID NO: 283 and SEQ ID NO: 288; SEQ ID NO: 283 and SEQ ID NO: 289; SEQ ID NO: 283 and SEQ ID NO: 290; SEQ ID NO: 283 and SEQ ID NO: 291; SEQ ID NO: 283 and SEQ ID NO: 292; SEQ ID NO: 284 and SEQ ID NO: 285; SEQ ID NO: 284 and SEQ ID NO: 286; SEQ ID NO: 284 and SEQ ID NO: 287; SEQ ID NO: 284 and SEQ ID NO: 288; SEQ ID NO: 284 and SEQ ID NO: 289; SEQ ID NO: 284 and SEQ ID NO: 290; SEQ ID NO: 284 and SEQ ID NO: 291; SEQ ID NO: 284 and SEQ ID NO: 292; SEQ ID NO: 285 and SEQ ID NO: 286; SEQ ID NO: 285 and SEQ ID NO: 287; SEQ ID NO: 285 and SEQ ID NO: 288; SEQ ID NO: 285 and SEQ ID NO: 289; SEQ ID NO: 285 and SEQ ID NO: 290; SEQ ID NO: 285 and SEQ ID NO: 291; SEQ ID NO: 285 and SEQ ID NO: 292; SEQ ID NO: 286 and SEQ ID NO: 287; SEQ ID NO: 286 and SEQ ID NO: 288; SEQ ID NO: 286 and SEQ ID NO: 289; SEQ ID NO: 286 and SEQ ID NO: 290; SEQ ID NO: 286 and SEQ ID NO: 291; SEQ ID NO: 286 and SEQ ID NO: 292; SEQ ID NO: 287 and SEQ ID NO: 288; SEQ ID NO: 287 and SEQ ID NO: 289; SEQ ID NO: 287 and SEQ ID NO: 290; SEQ ID NO: 287 and SEQ ID NO: 291; SEQ ID NO: 287 and SEQ ID NO: 292; SEQ ID NO: 288 and SEQ ID NO: 289; SEQ ID NO: 288 and SEQ ID NO: 290; SEQ ID NO: 288 and SEQ ID NO: 291; SEQ ID NO: 288 and SEQ ID NO: 292; SEQ ID NO: 289 and SEQ ID NO: 290; SEQ ID NO: 289 and SEQ ID NO: 291; SEQ ID NO: 289 and SEQ ID NO: 292; SEQ ID NO: 290 and SEQ ID NO: 291; SEQ ID NO: 290 and SEQ ID NO: 292; SEQ ID NO: 291 and SEQ ID NO: 292; SEQ ID NO: 270 and SEQ ID NO: 924; SEQ ID NO: 270 and SEQ ID NO: 925; SEQ ID NO: 270 and SEQ ID NO: 926; SEQ ID NO: 270 and SEQ ID NO: 927; SEQ ID NO: 270 and SEQ ID NO: 928; SEQ ID NO: 270 and SEQ ID NO: 929; SEQ ID NO: 270 and SEQ ID NO: 930; SEQ ID NO: 270 and SEQ ID NO: 931; SEQ ID NO: 270 and SEQ ID NO: 932; SEQ ID NO: 270 and SEQ ID NO: 933; SEQ ID NO: 270 and SEQ ID NO: 934; SEQ ID NO: 270 and SEQ ID NO: 935; SEQ ID NO: 270 and SEQ ID NO: 936; SEQ ID NO: 270 and SEQ ID NO: 937; SEQ ID NO: 270 and SEQ ID NO: 938; SEQ ID NO: 272 and SEQ ID NO: 924; SEQ ID NO: 272 and SEQ ID NO: 925; SEQ ID NO: 272 and SEQ ID NO: 926; SEQ ID NO: 272 and SEQ ID NO: 927; SEQ ID NO: 272 and SEQ ID NO: 928; SEQ ID NO: 272 and SEQ ID NO: 929; SEQ ID NO: 272 and SEQ ID NO: 930; SEQ ID NO: 272 and SEQ ID NO: 931; SEQ ID NO: 272 and SEQ ID NO: 932; SEQ ID NO: 272 and SEQ ID NO: 933; SEQ ID NO: 272 and SEQ ID NO: 934; SEQ ID NO: 272 and SEQ ID NO: 935; SEQ ID NO: 272 and SEQ ID NO: 936; SEQ ID NO: 272 and SEQ ID NO: 937; SEQ ID NO: 272 and SEQ ID NO: 938; SEQ ID NO: 273 and SEQ ID NO: 924; SEQ ID NO: 273 and SEQ ID NO: 925; SEQ ID NO: 273 and SEQ ID NO: 926; SEQ ID NO: 273 and SEQ ID NO: 927; SEQ ID NO: 273 and SEQ ID NO: 928; SEQ ID NO: 273 and SEQ ID NO: 929; SEQ ID NO: 273 and SEQ ID NO: 930; SEQ ID NO: 273 and SEQ ID NO: 931; SEQ ID NO: 273 and SEQ ID NO: 932; SEQ ID NO: 273 and SEQ ID NO: 933; SEQ ID NO: 273 and SEQ ID NO: 934; SEQ ID NO: 273 and SEQ ID NO: 935; SEQ ID NO: 273 and SEQ ID NO: 936; SEQ ID NO: 273 and SEQ ID NO: 937; SEQ ID NO: 273 and SEQ ID NO: 938; SEQ ID NO: 274 and SEQ ID NO: 924; SEQ ID NO: 274 and SEQ ID NO: 925; SEQ ID NO: 274 and SEQ ID NO: 926; SEQ ID NO: 274 and SEQ ID NO: 927; SEQ ID NO: 274 and SEQ ID NO: 928; SEQ ID NO: 274 and SEQ ID NO: 929; SEQ ID NO: 274 and SEQ ID NO: 930; SEQ ID NO: 274 and SEQ ID NO: 931; SEQ ID NO: 274 and SEQ ID NO: 932; SEQ ID NO: 274 and SEQ ID NO: 933; SEQ ID NO: 274 and SEQ ID NO: 934; SEQ ID NO: 274 and SEQ ID NO: 935; SEQ ID NO: 274 and SEQ ID NO: 936; SEQ ID NO: 274 and SEQ ID NO: 937; SEQ ID NO: 274 and SEQ ID NO: 938; SEQ ID NO: 275 and SEQ ID NO: 924; SEQ ID NO: 275 and SEQ ID NO: 925; SEQ ID NO: 275 and SEQ ID NO: 926; SEQ ID NO: 275 and SEQ ID NO: 927; SEQ ID NO: 275 and SEQ ID NO: 928; SEQ ID NO: 275 and SEQ ID NO: 929; SEQ ID NO: 275 and SEQ ID NO: 930; SEQ ID NO: 275 and SEQ ID NO: 931; SEQ ID NO: 275 and SEQ ID NO: 932; SEQ ID NO: 275 and SEQ ID NO: 933; SEQ ID NO: 275 and SEQ ID NO: 934; SEQ ID NO: 275 and SEQ ID NO: 935; SEQ ID NO: 275 and SEQ ID NO: 936; SEQ ID NO: 275 and SEQ ID NO: 937; SEQ ID NO: 275 and SEQ ID NO: 938; SEQ ID NO: 276 and SEQ ID NO: 924; SEQ ID NO: 276 and SEQ ID NO: 925; SEQ ID NO: 276 and SEQ ID NO: 926; SEQ ID NO: 276 and SEQ ID NO: 927; SEQ ID NO: 276 and SEQ ID NO: 928; SEQ ID NO: 276 and SEQ ID NO: 929; SEQ ID NO: 276 and SEQ ID NO: 930; SEQ ID NO: 276 and SEQ ID NO: 931; SEQ ID NO: 276 and SEQ ID NO: 932; SEQ ID NO: 276 and SEQ ID NO: 933; SEQ ID NO: 276 and SEQ ID NO: 934; SEQ ID NO: 276 and SEQ ID NO: 935; SEQ ID NO: 276 and SEQ ID NO: 936; SEQ ID NO: 276 and SEQ ID NO: 937; SEQ ID NO: 276 and SEQ ID NO: 938; SEQ ID NO: 277 and SEQ ID NO: 924; SEQ ID NO: 277 and SEQ ID NO: 925; SEQ ID NO: 277 and SEQ ID NO: 926; SEQ ID NO: 277 and SEQ ID NO: 927; SEQ ID NO: 277 and SEQ ID NO: 928; SEQ ID NO: 277 and SEQ ID NO: 929; SEQ ID NO: 277 and SEQ ID NO: 930; SEQ ID NO: 277 and SEQ ID NO: 931; SEQ ID NO: 277 and SEQ ID NO: 932; SEQ ID NO: 277 and SEQ ID NO: 933; SEQ ID NO: 277 and SEQ ID NO: 934; SEQ ID NO: 277 and SEQ ID NO: 935; SEQ ID NO: 277 and SEQ ID NO: 936; SEQ ID NO: 277 and SEQ ID NO: 937; SEQ ID NO: 277 and SEQ ID NO: 938; SEQ ID NO: 278 and SEQ ID NO: 924; SEQ ID NO: 278 and SEQ ID NO: 925; SEQ ID NO: 278 and SEQ ID NO: 926; SEQ ID NO: 278 and SEQ ID NO: 927; SEQ ID NO: 278 and SEQ ID NO: 928; SEQ ID NO: 278 and SEQ ID NO: 929; SEQ ID NO: 278 and SEQ ID NO: 930; SEQ ID NO: 278 and SEQ ID NO: 931; SEQ ID NO: 278 and SEQ ID NO: 932; SEQ ID NO: 278 and SEQ ID NO: 933; SEQ ID NO: 278 and SEQ ID NO: 934; SEQ ID NO: 278 and SEQ ID NO: 935; SEQ ID NO: 278 and SEQ ID NO: 936; SEQ ID NO: 278 and SEQ ID NO: 937; SEQ ID NO: 278 and SEQ ID NO: 938; SEQ ID NO: 279 and SEQ ID NO: 924; SEQ ID NO: 279 and SEQ ID NO: 925; SEQ ID NO: 279 and SEQ ID NO: 926; SEQ ID NO: 279 and SEQ ID NO: 927; SEQ ID NO: 279 and SEQ ID NO: 928; SEQ ID NO: 279 and SEQ ID NO: 929; SEQ ID NO: 279 and SEQ ID NO: 930; SEQ ID NO: 279 and SEQ ID NO: 931; SEQ ID NO: 279 and SEQ ID NO: 932; SEQ ID NO: 279 and SEQ ID NO: 933; SEQ ID NO: 279 and SEQ ID NO: 934; SEQ ID NO: 279 and SEQ ID NO: 935; SEQ ID NO: 279 and SEQ ID NO: 936; SEQ ID NO: 279 and SEQ ID NO: 937; SEQ ID NO: 279 and SEQ ID NO: 938; SEQ ID NO: 280 and SEQ ID NO: 924; SEQ ID NO: 280 and SEQ ID NO: 925; SEQ ID NO: 280 and SEQ ID NO: 926; SEQ ID NO: 280 and SEQ ID NO: 927; SEQ ID NO: 280 and SEQ ID NO: 928; SEQ ID NO: 280 and SEQ ID NO: 929; SEQ ID NO: 280 and SEQ ID NO: 930; SEQ ID NO: 280 and SEQ ID NO: 931; SEQ ID NO: 280 and SEQ ID NO: 932; SEQ ID NO: 280 and SEQ ID NO: 933; SEQ ID NO: 280 and SEQ ID NO: 934; SEQ ID NO: 280 and SEQ ID NO: 935; SEQ ID NO: 280 and SEQ ID NO: 936; SEQ ID NO: 280 and SEQ ID NO: 937; SEQ ID NO: 280 and SEQ ID NO: 938; SEQ ID NO: 281 and SEQ ID NO: 924; SEQ ID NO: 281 and SEQ ID NO: 925; SEQ ID NO: 281 and SEQ ID NO: 926; SEQ ID NO: 281 and SEQ ID NO: 927; SEQ ID NO: 281 and SEQ ID NO: 928; SEQ ID NO: 281 and SEQ ID NO: 929; SEQ ID NO: 281 and SEQ ID NO: 930; SEQ ID NO: 281 and SEQ ID NO: 931; SEQ ID NO: 281 and SEQ ID NO: 932; SEQ ID NO: 281 and SEQ ID NO: 933; SEQ ID NO: 281 and SEQ ID NO: 934; SEQ ID NO: 281 and SEQ ID NO: 935; SEQ ID NO: 281 and SEQ ID NO: 936; SEQ ID NO: 281 and SEQ ID NO: 937; SEQ ID NO: 281 and SEQ ID NO: 938; SEQ ID NO: 282 and SEQ ID NO: 924; SEQ ID NO: 282 and SEQ ID NO: 925; SEQ ID NO: 282 and SEQ ID NO: 926; SEQ ID NO: 282 and SEQ ID NO: 927; SEQ ID NO: 282 and SEQ ID NO: 928; SEQ ID NO: 282 and SEQ ID NO: 929; SEQ ID NO: 282 and SEQ ID NO: 930; SEQ ID NO: 282 and SEQ ID NO: 931; SEQ ID NO: 282 and SEQ ID NO: 932; SEQ ID NO: 282 and SEQ ID NO: 933; SEQ ID NO: 282 and SEQ ID NO: 934; SEQ ID NO: 282 and SEQ ID NO: 935; SEQ ID NO: 282 and SEQ ID NO: 936; SEQ ID NO: 282 and SEQ ID NO: 937; SEQ ID NO: 282 and SEQ ID NO: 938; SEQ ID NO: 283 and SEQ ID NO: 924; SEQ ID NO: 283 and SEQ ID NO: 925; SEQ ID NO: 283 and SEQ ID NO: 926; SEQ ID NO: 283 and SEQ ID NO: 927; SEQ ID NO: 283 and SEQ ID NO: 928; SEQ ID NO: 283 and SEQ ID NO: 929; SEQ ID NO: 283 and SEQ ID NO: 930; SEQ ID NO: 283 and SEQ ID NO: 931; SEQ ID NO: 283 and SEQ ID NO: 932; SEQ ID NO: 283 and SEQ ID NO: 933; SEQ ID NO: 283 and SEQ ID NO: 934; SEQ ID NO: 283 and SEQ ID NO: 935; SEQ ID NO: 283 and SEQ ID NO: 936; SEQ ID NO: 283 and SEQ ID NO: 937; SEQ ID NO: 283 and SEQ ID NO: 938; SEQ ID NO: 284 and SEQ ID NO: 924; SEQ ID NO: 284 and SEQ ID NO: 925; SEQ ID NO: 284 and SEQ ID NO: 926; SEQ ID NO: 284 and SEQ ID NO: 927; SEQ ID NO: 284 and SEQ ID NO: 928; SEQ ID NO: 284 and SEQ ID NO: 929; SEQ ID NO: 284 and SEQ ID NO: 930; SEQ ID NO: 284 and SEQ ID NO: 931; SEQ ID NO: 284 and SEQ ID NO: 932; SEQ ID NO: 284 and SEQ ID NO: 933; SEQ ID NO: 284 and SEQ ID NO: 934; SEQ ID NO: 284 and SEQ ID NO: 935; SEQ ID NO: 284 and SEQ ID NO: 936; SEQ ID NO: 284 and SEQ ID NO: 937; SEQ ID NO: 284 and SEQ ID NO: 938; SEQ ID NO: 285 and SEQ ID NO: 924; SEQ ID NO: 285 and SEQ ID NO: 925; SEQ ID NO: 285 and SEQ ID NO: 926; SEQ ID NO: 285 and SEQ ID NO: 927; SEQ ID NO: 285 and SEQ ID NO: 928; SEQ ID NO: 285 and SEQ ID NO: 929; SEQ ID NO: 285 and SEQ ID NO: 930; SEQ ID NO: 285 and SEQ ID NO: 931; SEQ ID NO: 285 and SEQ ID NO: 932; SEQ ID NO: 285 and SEQ ID NO: 933; SEQ ID NO: 285 and SEQ ID NO: 934; SEQ ID NO: 285 and SEQ ID NO: 935; SEQ ID NO: 285 and SEQ ID NO: 936; SEQ ID NO: 285 and SEQ ID NO: 937; SEQ ID NO: 285 and SEQ ID NO: 938; SEQ ID NO: 286 and SEQ ID NO: 924; SEQ ID NO: 286 and SEQ ID NO: 925; SEQ ID NO: 286 and SEQ ID NO: 926; SEQ ID NO: 286 and SEQ ID NO: 927; SEQ ID NO: 286 and SEQ ID NO: 928; SEQ ID NO: 286 and SEQ ID NO: 929; SEQ ID NO: 286 and SEQ ID NO: 930; SEQ ID NO: 286 and SEQ ID NO: 931; SEQ ID NO: 286 and SEQ ID NO: 932; SEQ ID NO: 286 and SEQ ID NO: 933; SEQ ID NO: 286 and SEQ ID NO: 934; SEQ ID NO: 286 and SEQ ID NO: 935; SEQ ID NO: 286 and SEQ ID NO: 936; SEQ ID NO: 286 and SEQ ID NO: 937; SEQ ID NO: 286 and SEQ ID NO: 938; SEQ ID NO: 287 and SEQ ID NO: 924; SEQ ID NO: 287 and SEQ ID NO: 925; SEQ ID NO: 287 and SEQ ID NO: 926; SEQ ID NO: 287 and SEQ ID NO: 927; SEQ ID NO: 287 and SEQ ID NO: 928; SEQ ID NO: 287 and SEQ ID NO: 929; SEQ ID NO: 287 and SEQ ID NO: 930; SEQ ID NO: 287 and SEQ ID NO: 931; SEQ ID NO: 287 and SEQ ID NO: 932; SEQ ID NO: 287 and SEQ ID NO: 933; SEQ ID NO: 287 and SEQ ID NO: 934; SEQ ID NO: 287 and SEQ ID NO: 935; SEQ ID NO: 287 and SEQ ID NO: 936; SEQ ID NO: 287 and SEQ ID NO: 937; SEQ ID NO: 287 and SEQ ID NO: 938; SEQ ID NO: 288 and SEQ ID NO: 924; SEQ ID NO: 288 and SEQ ID NO: 925; SEQ ID NO: 288 and SEQ ID NO: 926; SEQ ID NO: 288 and SEQ ID NO: 927; SEQ ID NO: 288 and SEQ ID NO: 928; SEQ ID NO: 288 and SEQ ID NO: 929; SEQ ID NO: 288 and SEQ ID NO: 930; SEQ ID NO: 288 and SEQ ID NO: 931; SEQ ID NO: 288 and SEQ ID NO: 932; SEQ ID NO: 288 and SEQ ID NO: 933; SEQ ID NO: 288 and SEQ ID NO: 934; SEQ ID NO: 288 and SEQ ID NO: 935; SEQ ID NO: 288 and SEQ ID NO: 936; SEQ ID NO: 288 and SEQ ID NO: 937; SEQ ID NO: 288 and SEQ ID NO: 938; SEQ ID NO: 289 and SEQ ID NO: 924; SEQ ID NO: 289 and SEQ ID NO: 925; SEQ ID NO: 289 and SEQ ID NO: 926; SEQ ID NO: 289 and SEQ ID NO: 927; SEQ ID NO: 289 and SEQ ID NO: 928; SEQ ID NO: 289 and SEQ ID NO: 929; SEQ ID NO: 289 and SEQ ID NO: 930; SEQ ID NO: 289 and SEQ ID NO: 931; SEQ ID NO: 289 and SEQ ID NO: 932; SEQ ID NO: 289 and SEQ ID NO: 933; SEQ ID NO: 289 and SEQ ID NO: 934; SEQ ID NO: 289 and SEQ ID NO: 935; SEQ ID NO: 289 and SEQ ID NO: 936; SEQ ID NO: 289 and SEQ ID NO: 937; SEQ ID NO: 289 and SEQ ID NO: 938; SEQ ID NO: 290 and SEQ ID NO: 924; SEQ ID NO: 290 and SEQ ID NO: 925; SEQ ID NO: 290 and SEQ ID NO: 926; SEQ ID NO: 290 and SEQ ID NO: 927; SEQ ID NO: 290 and SEQ ID NO: 928; SEQ ID NO: 290 and SEQ ID NO: 929; SEQ ID NO: 290 and SEQ ID NO: 930; SEQ ID NO: 290 and SEQ ID NO: 931; SEQ ID NO: 290 and SEQ ID NO: 932; SEQ ID NO: 290 and SEQ ID NO: 933; SEQ ID NO: 290 and SEQ ID NO: 934; SEQ ID NO: 290 and SEQ ID NO: 935; SEQ ID NO: 290 and SEQ ID NO: 936; SEQ ID NO: 290 and SEQ ID NO: 937; SEQ ID NO: 290 and SEQ ID NO: 938; SEQ ID NO: 291 and SEQ ID NO: 924; SEQ ID NO: 291 and SEQ ID NO: 925; SEQ ID NO: 291 and SEQ ID NO: 926; SEQ ID NO: 291 and SEQ ID NO: 927; SEQ ID NO: 291 and SEQ ID NO: 928; SEQ ID NO: 291 and SEQ ID NO: 929; SEQ ID NO: 291 and SEQ ID NO: 930; SEQ ID NO: 291 and SEQ ID NO: 931; SEQ ID NO: 291 and SEQ ID NO: 932; SEQ ID NO: 291 and SEQ ID NO: 933; SEQ ID NO: 291 and SEQ ID NO: 934; SEQ ID NO: 291 and SEQ ID NO: 935; SEQ ID NO: 291 and SEQ ID NO: 936; SEQ ID NO: 291 and SEQ ID NO: 937; SEQ ID NO: 291 and SEQ ID NO: 938; SEQ ID NO: 292 and SEQ ID NO: 924; SEQ ID NO: 292 and SEQ ID NO: 925; SEQ ID NO: 292 and SEQ ID NO: 926; SEQ ID NO: 292 and SEQ ID NO: 927; SEQ ID NO: 292 and SEQ ID NO: 928; SEQ ID NO: 292 and SEQ ID NO: 929; SEQ ID NO: 292 and SEQ ID NO: 930; SEQ ID NO: 292 and SEQ ID NO: 931; SEQ ID NO: 292 and SEQ ID NO: 932; SEQ ID NO: 292 and SEQ ID NO: 933; SEQ ID NO: 292 and SEQ ID NO: 934; SEQ ID NO: 292 and SEQ ID NO: 935; SEQ ID NO: 292 and SEQ ID NO: 936; SEQ ID NO: 292 and SEQ ID NO: 937; SEQ ID NO: 292 and SEQ ID NO: 938; SEQ ID NO: 950 and SEQ ID NO: 275; SEQ ID NO: 951 and SEQ ID NO: 275; SEQ ID NO: 952 and SEQ ID NO: 275; SEQ ID NO: 953 and SEQ ID NO: 275; SEQ ID NO: 954 and SEQ ID NO: 275; or SEQ ID NO: 955 and SEQ ID NO: 275.

4. (canceled)

5. The composition of claim 1, comprising a pair of guide RNAs, wherein the pair of guide RNAs is capable of excising a portion of the DMD gene; wherein the portion of the DMD gene is between 5-250 nucleotides in length.

6.-12. (canceled)

13. A composition comprising a first and a second nucleic acid molecule, wherein the first nucleic acid molecule encodes a sRGN endonuclease (e.g., sRGN3.1, sRGN3.3, or sRGN4) and optionally a first or a first and second guide RNA, and the second nucleic acid molecule comprises a first or a first and second guide RNA, wherein the first guide RNA comprises a first sequence and the second guide RNAs comprises a second sequence from any one the following pairs of first and second sequences: SEQ ID NOs: 200 and 201; 200 and 202; 200 and 203; 200 and 204; 200 and 205; 200 and 206; 200 and 207; 200 and 208; 201 and 202; 201 and 203; 201 and 204; 201 and 205; 201 and 206; 201 and 207; 201 and 208; 202 and 203; 202 and 204; 202 and 205; 202 and 206; 202 and 207; 202 and 208; 203 and 204; 203 and 205; 203 and 206; 203 and 207; 203 and 208; 204 and 205; 204 and 206; 204 and 207; 204 and 208; 205 and 206; 205 and 207; 205 and 208; 206 and 207; 206 and 208; 207 and 208; 231 and 232; 231 and 233; 231 and 234; 231 and 235; 231 and 236; 231 and 237; 231 and 238; 231 and 239; 231 and 240; 231 and 241; 231 and 242; 232 and 233; 232 and 234; 232 and 235; 232 and 236; 232 and 237; 232 and 238; 232 and 239; 232 and 240; 232 and 241; 232 and 242; 233 and 234; 233 and 235; 233 and 236; 233 and 237; 233 and 238; 233 and 239; 233 and 240; 233 and 241; 233 and 242; 234 and 235; 234 and 236; 234 and 237; 234 and 238; 234 and 239; 234 and 240; 234 and 241; 234 and 242; 235 and 236; 235 and 237; 235 and 238; 235 and 239; 235 and 240; 235 and 241; 235 and 242; 236 and 237; 236 and 238; 236 and 239; 236 and 240; 236 and 241; 236 and 242; 237 and 238; 237 and 239; 237 and 240; 237 and 241; 237 and 242; 238 and 239; 238 and 240; 238 and 241; 238 and 242; 239 and 240; 239 and 241; 239 and 242; 240 and 241; 240 and 242; 241 and 242; 270 and 271; 270 and 272; 270 and 273; 270 and 274; 270 and 275; 270 and 276; 270 and 277; 270 and 278; 270 and 279; 270 and 280; 270 and 281; 270 and 282; 270 and 283; 270 and 284; 270 and 285; 270 and 286; 270 and 287; 270 and 288; 270 and 289; 270 and 290; 270 and 291; 270 and 292; 271 and 272; 271 and 273; 271 and 274; 271 and 275; 271 and 276; 271 and 277; 271 and 278; 271 and 279; 271 and 280; 271 and 281; 271 and 282; 271 and 283; 271 and 284; 271 and 285; 271 and 286; 271 and 287; 271 and 288; 271 and 289; 271 and 290; 271 and 291; 271 and 292; 272 and 273; 272 and 274; 272 and 275; 272 and 276; 272 and 277; 272 and 278; 272 and 279; 272 and 280; 272 and 281; 272 and 282; 272 and 283; 272 and 284; 272 and 285; 272 and 286; 272 and 287; 272 and 288; 272 and 289; 272 and 290; 272 and 291; 272 and 292; 273 and 274; 273 and 275; 273 and 276; 273 and 277; 273 and 278; 273 and 279; 273 and 280; 273 and 281; 273 and 282; 273 and 283; 273 and 284; 273 and 285; 273 and 286; 273 and 287; 273 and 288; 273 and 289; 273 and 290; 273 and 291; 273 and 292; 274 and 275; 274 and 276; 274 and 277; 274 and 278; 274 and 279; 274 and 280; 274 and 281; 274 and 282; 274 and 283; 274 and 284; 274 and 285; 274 and 286; 274 and 287; 274 and 288; 274 and 289; 274 and 290; 274 and 291; 274 and 292; 275 and 276; 275 and 277; 275 and 278; 275 and 279; 275 and 280; 275 and 281; 275 and 282; 275 and 283; 275 and 284; 275 and 285; 275 and 286; 275 and 287; 275 and 288; 275 and 289; 275 and 290; 275 and 291; 275 and 292; 276 and 277; 276 and 278; 276 and 279; 276 and 280; 276 and 281; 276 and 282; 276 and 283; 276 and 284; 276 and 285; 276 and 286; 276 and 287; 276 and 288; 276 and 289; 276 and 290; 276 and 291; 276 and 292; 277 and 278; 277 and 279; 277 and 280; 277 and 281; 277 and 282; 277 and 283; 277 and 284; 277 and 285; 277 and 286; 277 and 287; 277 and 288; 277 and 289; 277 and 290; 277 and 291; 277 and 292; 278 and 279; 278 and 280; 278 and 281; 278 and 282; 278 and 283; 278 and 284; 278 and 285; 278 and 286; 278 and 287; 278 and 288; 278 and 289; 278 and 290; 278 and 291; 278 and 292; 279 and 280; 279 and 281; 279 and 282; 279 and 283; 279 and 284; 279 and 285; 279 and 286; 279 and 287; 279 and 288; 279 and 289; 279 and 290; 279 and 291; 279 and 292; 280 and 281; 280 and 282; 280 and 283; 280 and 284; 280 and 285; 280 and 286; 280 and 287; 280 and 288; 280 and 289; 280 and 290; 280 and 291; 280 and 292; 281 and 282; 281 and 283; 281 and 284; 281 and 285; 281 and 286; 281 and 287; 281 and 288; 281 and 289; 281 and 290; 281 and 291; 281 and 292; 282 and 283; 282 and 284; 282 and 285; 282 and 286; 282 and 287; 282 and 288; 282 and 289; 282 and 290; 282 and 291; 282 and 292; 283 and 284; 283 and 285; 283 and 286; 283 and 287; 283 and 288; 283 and 289; 283 and 290; 283 and 291; 283 and 292; 284 and 285; 284 and 286; 284 and 287; 284 and 288; 284 and 289; 284 and 290; 284 and 291; 284 and 292; 285 and 286; 285 and 287; 285 and 288; 285 and 289; 285 and 290; 285 and 291; 285 and 292; 286 and 287; 286 and 288; 286 and 289; 286 and 290; 286 and 291; 286 and 292; 287 and 288; 287 and 289; 287 and 290; 287 and 291; 287 and 292; 288 and 289; 288 and 290; 288 and 291; 288 and 292; 289 and 290; 289 and 291; 289 and 292; 290 and 291; 290 and 292; 291 and 292; 924 and 270; 924 and 272; 924 and 273; 924 and 274; 924 and 275; 924 and 276; 924 and 277; 924 and 278; 924 and 279; 924 and 280; 924 and 281; 924 and 282; 924 and 283; 924 and 284; 924 and 285; 924 and 286; 924 and 287; 924 and 288; 924 and 289; 924 and 290; 924 and 291; 924 and 292; 925 and 270; 925 and 272; 925 and 273; 925 and 274; 925 and 275; 925 and 276; 925 and 277; 925 and 278; 925 and 279; 925 and 280; 925 and 281; 925 and 282; 925 and 283; 925 and 284; 925 and 285; 925 and 286; 925 and 287; 925 and 288; 925 and 289; 925 and 290; 925 and 291; 925 and 292; 926 and 270; 926 and 272; 926 and 273; 926 and 274; 926 and 275; 926 and 276; 926 and 277; 926 and 278; 926 and 279; 926 and 280; 926 and 281; 926 and 282; 926 and 283; 926 and 284; 926 and 285; 926 and 286; 926 and 287; 926 and 288; 926 and 289; 926 and 290; 926 and 291; 926 and 292; 927 and 270; 927 and 272; 927 and 273; 927 and 274; 927 and 275; 927 and 276; 927 and 277; 927 and 278; 927 and 279; 927 and 280; 927 and 281; 927 and 282; 927 and 283; 927 and 284; 927 and 285; 927 and 286; 927 and 287; 927 and 288; 927 and 289; 927 and 290; 927 and 291; 927 and 292; 928 and 270; 928 and 272; 928 and 273; 928 and 274; 928 and 275; 928 and 276; 928 and 277; 928 and 278; 928 and 279; 928 and 280; 928 and 281; 928 and 282; 928 and 283; 928 and 284; 928 and 285; 928 and 286; 928 and 287; 928 and 288; 928 and 289; 928 and 290; 928 and 291; 928 and 292; 929 and 270; 929 and 272; 929 and 273; 929 and 274; 929 and 275; 929 and 276; 929 and 277; 929 and 278; 929 and 279; 929 and 280; 929 and 281; 929 and 282; 929 and 283; 929 and 284; 929 and 285; 929 and 286; 929 and 287; 929 and 288; 929 and 289; 929 and 290; 929 and 291; 929 and 292; 930 and 270; 930 and 272; 930 and 273; 930 and 274; 930 and 275; 930 and 276; 930 and 277; 930 and 278; 930 and 279; 930 and 280; 930 and 281; 930 and 282; 930 and 283; 930 and 284; 930 and 285; 930 and 286; 930 and 287; 930 and 288; 930 and 289; 930 and 290; 930 and 291; 930 and 292; 931 and 270; 931 and 272; 931 and 273; 931 and 274; 931 and 275; 931 and 276; 931 and 277; 931 and 278; 931 and 279; 931 and 280; 931 and 281; 931 and 282; 931 and 283; 931 and 284; 931 and 285; 931 and 286; 931 and 287; 931 and 288; 931 and 289; 931 and 290; 931 and 291; 931 and 292; 932 and 270; 932 and 272; 932 and 273; 932 and 274; 932 and 275; 932 and 276; 932 and 277; 932 and 278; 932 and 279; 932 and 280; 932 and 281; 932 and 282; 932 and 283; 932 and 284; 932 and 285; 932 and 286; 932 and 287; 932 and 288; 932 and 289; 932 and 290; 932 and 291; 932 and 292; 933 and 270; 933 and 272; 933 and 273; 933 and 274; 933 and 275; 933 and 276; 933 and 277; 933 and 278; 933 and 279; 933 and 280; 933 and 281; 933 and 282; 933 and 283; 933 and 284; 933 and 285; 933 and 286; 933 and 287; 933 and 288; 933 and 289; 933 and 290; 933 and 291; 933 and 292; 934 and 270; 934 and 272; 934 and 273; 934 and 274; 934 and 275; 934 and 276; 934 and 277; 934 and 278; 934 and 279; 934 and 280; 934 and 281; 934 and 282; 934 and 283; 934 and 284; 934 and 285; 934 and 286; 934 and 287; 934 and 288; 934 and 289; 934 and 290; 934 and 291; 934 and 292; 935 and 270; 935 and 272; 935 and 273; 935 and 274; 935 and 275; 935 and 276; 935 and 277; 935 and 278; 935 and 279; 935 and 280; 935 and 281; 935 and 282; 935 and 283; 935 and 284; 935 and 285; 935 and 286; 935 and 287; 935 and 288; 935 and 289; 935 and 290; 935 and 291; 935 and 292; 936 and 270; 936 and 272; 936 and 273; 936 and 274; 936 and 275; 936 and 276; 936 and 277; 936 and 278; 936 and 279; 936 and 280; 936 and 281; 936 and 282; 936 and 283; 936 and 284; 936 and 285; 936 and 286; 936 and 287; 936 and 288; 936 and 289; 936 and 290; 936 and 291; 936 and 292; 937 and 270; 937 and 272; 937 and 273; 937 and 274; 937 and 275; 937 and 276; 937 and 277; 937 and 278; 937 and 279; 937 and 280; 937 and 281; 937 and 282; 937 and 283; 937 and 284; 937 and 285; 937 and 286; 937 and 287; 937 and 288; 937 and 289; 937 and 290; 937 and 291; 937 and 292; 938 and 270; 938 and 272; 938 and 273; 938 and 274; 938 and 275; 938 and 276; 938 and 277; 938 and 278; 938 and 279; 938 and 280; 938 and 281; 938 and 282; 938 and 283; 938 and 284; 938 and 285; 938 and 286; 938 and 287; 938 and 288; 938 and 289; 938 and 290; 938 and 291; 938 and 292; 950 and 275; 951 and 275; 952 and 275; 953 and 275; 954 and 275; or 955 and 275.

14. The composition of claim 13, wherein (a) the sRGN endonuclease comprises an amino acid sequence that is at least 80%, 85%, 90%, 91%, 92%, 93%, 94%, 95%, 96%, 97%, 98%, 99%, or 100% identical to the sequence of any one of SEQ ID Nos: 7024, 7026, or 7027; or (b) the sRGN endonuclease is encoded by a nucleic acid sequence that is at least 80%, 85%, 90%, 91%, 92%, 93%, 94%, 95%, 96%, 97%, 98%, 99%, or 100% identical to the sequence of any one of SEQ ID Nos: 917, 919 or 920.

15. (canceled)

16. The composition of claim 1, comprising at least two guide RNAs, wherein once expressed in vitro or in vivo in combination with an RNA-guided endonuclease, the at least two guide RNAs
(a) facilitate a 3n+1 edit of exon 45, 51 or 53 of the DMD gene, wherein “n” is any negative whole number (e.g., any whole number between −10 and −75); or
(b) facilitate a 3n+2 edit of exon 44 or 50 of the DMD gene, wherein “n” is any negative whole number (e.g., any whole number between −10 and −75).

17. The composition of claim 1, comprising at least two guide RNAs, wherein once expressed in vitro or in vivo in combination with an RNA-guided endonuclease, the at least two guide RNAs
(a) target exon 45, 51 or 53 of the DMD gene and are capable of excising a nucleic acid that is at least 26, 29, 32, 35, 38, 41, 44, 47, 50, 53, 56, 59, 62, 65, 68, 71, 74, 77, 80, 83, 86, 89, 92, 95, 98, 101, 104, 107, 110, 113, 116, 119, 122, 125, 128, 131, 134, 137, 140, 143, 146, 149, 152, 155, 158, 161, 164, 167, 170, 173, 176, 179, 182, 185, 188, 191, 194, 197, 200, 203, 206, 209, 212, 215, or 218 nucleotides in length; or
(b) target exon 44 or 50 of the DMD gene and are capable of excising a nucleic acid that is at least 25, 28, 31, 34, 37, 40, 43, 46, 49, 52, 55, 58, 61, 64, 67, 70, 73, 76, 79, 82, 85, 88, 91, 94, 97, 100, 103, 106, 109, 112, 115, 118, 121, 124, 127, 130, 133, 136, 139, 142, 145, 148, 151, 154, 157, 160, 163, 166, 169, 172, 175, 178, 181, 184, 187, 190, 193, 196, 199, 202, 205, 208, 211, 214, or 217 nucleotides in length.

18.-20. (canceled)

21. The composition of claim 1, comprising at least two guide RNAs, wherein once expressed in vitro or in vivo in combination with an RNA-guided endonuclease, the at least two guide RNAs excise a portion of a DMD exon, wherein
(a) the size of the excised portion of the DMD exon is between 5 and 250, 5 and 200, 5 and 150, 5 and 100, 5 and 75, 5 and 50, 5 and 25, 5 and 10, 20 and 250, 20 and 200, 20 and 150, 20 and 100, 20 and 75, 20 and 50, 20 and 25, 50 and 250, 50 and 200, 50 and 150, 50 and 100, or 50 and 75 nucleotides; or
(b) the size of the excised portion of the DMD exon is between 8 and 167 nucleotides.

22. (canceled)

23. The composition of claim 1, wherein the one or more guide RNAs is an sgRNA.

24. The composition of claim 1, wherein the one or more guide RNAs is modified.

25. The composition of claim 1, wherein the one or more guide RNAs or nucleic acids are in a vector.

26. The composition of claim 25, wherein the vector is a viral vector.

27. The composition of claim 26, wherein the viral vector is an AAV vector.

28. The composition of claim 27, wherein the AAV vector is an AAV9 vector.

29. The composition of claim 1, wherein the promoter for the one or more guide RNAs is hU6c.

30. The composition of claim 1, wherein
(a) the one or more guide RNAs is a guide RNA for SaCas9, and the one or more guide RNAs comprise a scaffold comprising the sequence of SEQ ID NO: 504; or
(b) the one or more guide RNAs is a guide RNA for SluCas9, and the one or more guide RNAs comprise a scaffold comprising the sequence of SEQ ID NO: 901.

31. (canceled)

32. The composition of claim 1, wherein the one or more guide RNAs is in an AAV vector, wherein the vector comprises from 5′ to 3′ with respect to the plus strand: the reverse complement of a first guide RNA scaffold sequence; the reverse complement of a nucleic acid encoding a first guide RNA sequence; the reverse complement of a promoter for expression of the nucleic acid encoding the first guide RNA sequence; a promoter for expression of a nucleic acid encoding a SaCas9, a SluCas9, or a sRGN (e.g., CK8e); a nucleic acid encoding the SaCas9, the SluCas9, or the sRGN (e.g., sRGN3.1, sRGN3,3, or sRGN4); a polyadenylation sequence; a promoter for expression of a second guide RNA sequence in the same direction as the promoter for the SaCas9, the SluCas9, or the sRGN; a nucleic acid encoding a second RNA guide sequence; and a second guide RNA scaffold sequence.

33. The composition of claim 32, wherein:
I.
i) the first guide RNA sequence comprises the sequence of SEQ TD NO: 271 or 281, and the second guide RNA sequence comprises the sequence of SEQ TD NO: 275; or
ii) the first guide RNA sequence comprises the sequence of SEQ ID NO: 283, and the second guide RNA sequence comprises the sequence of SEQ ID NO: 290; and/or
II. the promoter for expression of the nucleic acid encoding the SaCas9, the SluCas9, or the sRGN is a promoter for expression of the sRGN: wherein the nucleic acid encoding the SaCas9, the SluCas9, or the sRGN encodes the sRGN, and wherein the sRGN comprises an amino acid sequence that is at least 80%, 85%, 90%, 91%, 92%, 93%, 94%, 95%, 96%, 97%, 98%, 99%, or 100% identical to the sequence of any one of SEQ ID NOs: 7024, 7026, or 7027.

34. (canceled)

35. The composition of claim 33, wherein the nucleic acid encoding the sRGN comprises a nucleic acid sequence that is at least 80%, 85%, 90%, 91%, 92%, 93%, 94%, 95%, 96%, 97%, 98%, 99%, or 100% identical to the sequence of any one of SEQ ID Nos: 917, 919 or 920.

36. The composition of claim 1, wherein the one or more guide RNA sequences is no more than 16, no more than 17, no more than 18, no more than 19, no more than 20, no more than 21, or no more than 22 nucleotides in length.

37. A method of treating Duchenne Muscular Dystrophy (DMD), the method comprising delivering to a cell the composition of claim 1.

38. A method of excising a portion of the DMD gene, the method comprising delivering to a cell the composition of claim 1, wherein the composition comprises a pair of guide RNAs that are capable of excising a portion of the DMD gene, and wherein the size of the excised portion of the DMD gene is less than about 250 nucleotides.
